(19) United States
(12) Patent Application Publication
(10) Pub. No.: US 2026/0125400 A1
(43) Pub. Date: May 7, 2026

(54) TYK2 DEGRADERS AND USES THEREOF

(71) Applicant: Kymera Therapeutics, Inc., Watertown, MA (US)

(72) Inventors: Isaac Marx, Watertown, MA (US); Xiao Zhu, Watertown, MA (US); Christopher M. Yates, Watertown, MA (US); Bin Yang, Watertown, MA (US); Xiaozhang Zheng, Watertown, MA (US); Michael D. Sintchak, Watertown, MA (US); Yi Zhang, Watertown, MA (US); Matthew M. Weiss, Watertown, MA (US); Lewis Dale Pennington, Watertown, MA (US); Nan Ji, Watertown, MA (US); Eamon Comer, Watertown, MA (US); Melissa Ford, Watertown, MA (US)

(21) Appl. No.: 19/476,833

(22) PCT Filed: Apr. 19, 2024
(86) PCT No.: PCT/US2024/025584

§ 371 Date: Oct. 20, 2025

Related U.S. Application Data
(60) Provisional application No. 63/497,504, filed on Apr. 21, 2023.
(60) Provisional application No. 63/587,879, filed on Oct. 4, 2023.
(60) Provisional application No. 63/596,205, filed on Nov. 3, 2023.
(60) Provisional application No. 63/617,188, filed on Jan. 3, 2024.

(51) Int. Cl. C07D 519/00; A61K 31/4545; A61K 31/496; A61K 31/501; A61K 31/5025; A61K 31/506; A61K 31/513; A61K 31/519; A61K 31/5377; A61K 31/5383; A61K 31/55; C07B 59/00; C07D 401/14; C07D 471/04; C07D 471/10; C07D 487/04

(52) U.S. Cl. CPC C07D 519/00; A61K 31/4545; A61K 31/496; A61K 31/501; A61K 31/5025; A61K 31/506; A61K 31/513; A61K 31/519; A61K 31/5377; A61K 31/5383; A61K 31/55; C07B 59/002; C07D 401/14; C07D 471/04; C07D 471/10; C07D 487/04; C07B 2200/05

(57) ABSTRACT

The present invention provides compounds, compositions thereof, and methods of using the same.

### CROSS REFERENCE TO RELATED APPLICATION

The application claims the benefit of priority of U.S. Provisional Appl. No. 63/497,504, filed Apr. 21, 2023, U.S. Provisional Appl. No. 63/587,879, filed Oct. 4, 2023, U.S. Provisional Appl. No. 63/596,205, filed Nov. 3, 2023, and U.S. Provisional Appl. No. 63/617,188, filed Jan. 3, 2024, the content of each of which in herein incorporated by reference.

### TECHNICAL FIELD OF THE INVENTION

The present invention relates to compounds and methods useful for the modulation of tyrosine kinase 2 (“TYK2”) protein via ubiquitination and/or degradation by compounds according to the present invention. The invention also provides pharmaceutically acceptable compositions comprising compounds of the present invention and methods of using said compositions in the treatment of various disorders.

### BACKGROUND OF THE INVENTION

Ubiquitin-Proteasome Pathway (UPP) or Ubiquitin-Proteasome System (UPS) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17(6):551-555; Schnnekloth J S Jr., Chembiochem, 2005, 6(1):40-46).

TYK2 is an enzyme encoded by the TYK2 gene in humans and a member of the Janus Kinase (JAKs) family of proteins. TYK2 is involved IL-12, IL-23 and type I-interferon (IFN) signaling (Morris R, et al., Protein Science, Volume: 27, Issue: 12, Pages: 1984-2009, 2018). Human genetic studies suggest that TYK2 inhibition can be broadly beneficial for treating autoimmune and inflammatory diseases (Dendrou C, et al., Science Translational Medicine, Vol 8, Issue 363, p. 363ra149 2016).

An ongoing need exists in the art for effective treatments for diseases, especially autoimmune and inflammatory diseases and disorders mediated by pro-inflammatory molecules such as IFN-α/β, IL-12, and IL-23 without JAK 1/2 inhibition which may cause on-target adverse events. As such, small molecule therapeutic agents that leverage E3 ligase mediated protein degradation to pro-inflammatory associated proteins such as tyrosine kinase 2 (TYK2) while potentially sparing molecules involved in wound healing and protection against microbes such as IL-10 and IL-22 hold promise as therapeutic agents for the treatment of conditions such as Crohn's disease and ulcerative colitis. Accordingly, there remains a need to find compounds that are TYK2 degraders useful as therapeutic agents.

### SUMMARY OF THE INVENTION

It has now been found that the heterobifunctional compounds of this invention, and pharmaceutically acceptable compositions thereof, are effective degraders of TYK2. In certain embodiments, the invention provides for compounds of the formulae presented herein.

Compounds of the present invention, and pharmaceutically acceptable compositions thereof, are useful for treating a variety of diseases, disorders or conditions, associated with modulating TYK2. Such diseases, disorders, or conditions include those described herein.

Compounds provided by this invention are also useful for the study of TYK2 kinase in biological and pathological phenomena; the study of intracellular signal transduction pathways occurring in bodily tissues; and the comparative evaluation of new TYK2 inhibitors or other regulators of kinases, signaling pathways, and cytokine levels in vitro or in vivo.

### DETAILED DESCRIPTION OF CERTAIN EMBODIMENTS

### 1. General Description of Certain Embodiments of the Invention

In certain embodiments, the present invention provides a compound of any one of the following formulae:

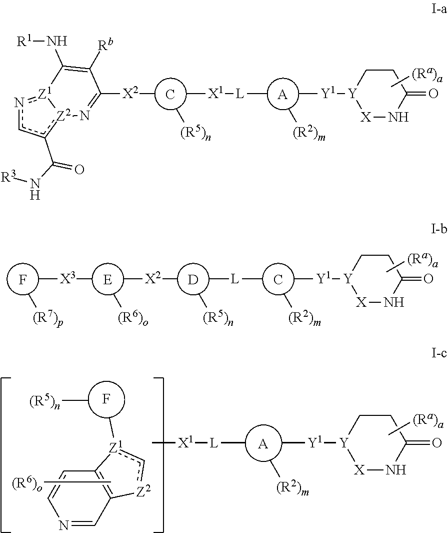

or a pharmaceutically acceptable salt thereof, wherein each variable is as defined and described herein, both singly and in combination.

In some embodiments, the present invention provides a pharmaceutical composition comprising a compound of formula I-a, I-c, or I-c and a pharmaceutically acceptable carrier, adjuvant, or diluent.

In some embodiments, the present invention provides a method of treating a TYK2-mediated disease, disorder, or condition comprising administering to a patient in need thereof, a compound of formula -a, I-c, or I-c, a pharmaceutically acceptable salt thereof, or a pharmaceutical composition thereof.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B, and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic, bicyclic, bridged bicyclic, or spirocyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted.

The term “alkyl” refers to a C₁₋₁₂ straight or branched saturated aliphatic group. In certain instances, alkyl refers to a C₁₋₈ straight or branched saturated aliphatic group or a C₁₋₆ straight or branched saturated aliphatic group. The term “lower alkyl” refers to a C₁₋₄ straight or branched alkyl group. Exemplary lower alkyl groups are methyl, ethyl, propyl, isopropyl (also referred to interchangeably herein as iPr, ⁱPr and i-Pr), butyl, isobutyl (also referred to interchangeably herein as iBu, ⁱBu and i-Bu) and tert-butyl (also referred to interchangeably herein as tBu, ^{t}Bu and t-Bu).

The term “alkenyl” refers to a C₁₋₁₂ straight or branched partially unsaturated aliphatic group comprising at least one unsaturated carbon carbon double bond. In certain instances, alkenyl refers to a C₂₋₈ or a C₁₋₆ straight or branched partially unsaturated aliphatic group comprising at least one unsaturated carbon carbon double bond. The term “lower alkenyl” refers to a C₂₋₄ straight or branched partially unsaturated aliphatic group comprising at least one unsaturated carbon carbon double bond. Alkenyl groups include both cis (Z) and trans (E) regioisomers. Exemplary lower alkenyl groups are vinyl, allyl, 2-propenyl, and butenyl isomers (—CH₂CH₂CH═CH₂, —CH₂CH═CHCH₃ and —CH═CHCH₂CH₃).

The term “alkynyl” refers to a C2-12 straight or branched partially unsaturated aliphatic group comprising at least one unsaturated carbon carbon triple bond. In certain instances, alkynyl refers to a C2-8 or a C1-6 straight or branched partially unsaturated aliphatic group comprising at least one unsaturated carbon carbon triple bond. The term “lower alkynyl” refers to a C2-4 straight or branched partially unsaturated aliphatic group comprising at least one unsaturated carbon carbon triple bond. Exemplary lower alkynyl groups are ethynyl, 1-propynyl, 2-propynyl, 1-butynyl, 2-butynyl, and 3-butynyl.

The term “haloalkyl” refers to a straight or branched alkyl group that is substituted with one or more halogen atoms. The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

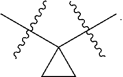 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention. “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, pyrazinyl, pyrazolyl, pyridazinyl, pyridyl, pyrimidinyl, triazinyl, thienyl, furanyl, pyrrolyl, imidazolyl, pyrazolyl, triazolyl (i.e., 1,2,3-triazolyl), 1,2,4-triazolyl, 1,2,5-triazolyl, 1,3,4-triazolyl, tetrazolyl, oxazolyl, isoxazolyl, oxadiazolyl, 1,2,3-oxadiazolyl, 1,2,4-oxadiazolyl, 1,2,5-oxadiazolyl, 1,3,4-oxadiazolyl, thiazolyl, isothiazolyl, thiadiazolyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, indolizinyl, isoindolin-1-only, 1,2-dihydro-3H-pyrrolo[3,4-c]pyridin-3-onyl, 2,3-dihydro-1H-pyrrolo[3,4-c]pyridin-1-onyl, imidazo[1,2-a]pyridyl, imidazo[1,5-a]pyridyl, pyrazolo[1,5-a]pyridyl, pyrrolo[1,2-b]pyridazinyl, pyrrolo[1,2-a]pyrimidinyl, imidazo[1,2-b]pyridazinyl, imidazo[1,2-a]pyrimidinyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be monocyclic, bicyclic, or tricyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5 to 7-membered monocyclic or 7-10 membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, oxetanyl, azetidinyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic, bridged bicyclic, spirocyclic, or mixtures thereof. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}: —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄S(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —(CH₂)₀₋₄P(O)₂R^{∘}; —(CH₂)₀₋₄P(O)R^{∘}₂; —(CH₂)_{0- 4}P(O)(OR^{∘})₂; —(CH₂)₀₋₄OP(O)R^{∘}₂; —(CH₂)₀₋₄OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen. C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)O₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃₀—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen. —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR⁺, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted—OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{f} are independently halogen, —R*, -(haloR^{●}), —OH, —OR^{●}, —O(haloR^{●}), —CN, —C(O)OH, —C(O)OR^{●}, —NH₂, —NHR, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers and R^{a} (or M) and Sₐ (or θ) atropisomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the term “inhibitorily active metabolite or residue thereof” means that a metabolite or residue thereof is also an inhibitor of TYK2, or a mutant thereof.

As used herein, the term “degratorily active metabolite or residue thereof” means that a metabolite or residue thereof is also a degrader of TYK2, or a mutant thereof.

As used herein, the term “inhibitor” is defined as a compound that binds to and/or inhibits TYK2 with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “degrader” is defined as a heterobifunctional compound that binds to and/or inhibits both TYK2, and an E3 ligase with measurable affinity resulting in the ubiquitination and subsequent degradation of TYK2. In certain embodiments, a degrader has an DC₅₀ of less than about 50 μM, less than about 1 μM, less than about 500 nM, less than about 100 nM, less than about 10 nM, or less than about 1 nM.

The terms “measurable affinity” and “measurably inhibit,” as used herein, means a measurable change in TYK2 activity between a sample comprising a compound of the present invention, or composition thereof, and TYK2, and an equivalent sample comprising TYK2, in the absence of said compound, or composition thereof.

As used herein, the term “patient” refers to an animal, preferably a mammal, and most preferably a human.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. In some embodiments, the provided compounds are purified in salt form for convenience and/or ease of purification, e.g., using an acidic or basic mobile phase during chromatography. Salts forms of the provided compounds formed during chromatographic purification are contemplated herein and are readily apparent to those having skill in the art.

As used herein, the term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the term “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily or degratorily active metabolite or residue thereof.

As used herein, the term “provided compound” refers to any genus, subgenus, and/or species set forth herein.

### 3. Description of Exemplary Embodiments

As described above, in certain embodiments, the present invention provides a compound of formula I-a:

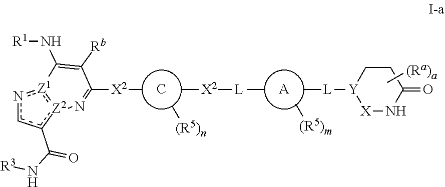

or a pharmaceutically acceptable salt thereof, wherein:
X is a bivalent moiety selected from —CH₂— or —C(O)—;
X¹ is a covalent bond,

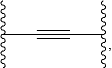 [SMILES: *C#C*]

or an optionally substituted ring selected from 4-6 membered saturated carbocyclylenyl or heterocyclylenyl having 1-2 heteroatoms independently selected from nitrogen, oxygen, and sulfur, phenylenyl, 5-6 membered heteroarylenyl having 1-2 heteroatoms independently selected from nitrogen, oxygen, and sulfur, and a 5-8 membered saturated bridged bicyclic heterocyclylenyl having 1-2 heteroatoms independently selected from nitrogen, oxygen, and sulfur;
X² is a covalent bond, —O—, or —NR—;
Y is N or CH;
Y¹ is a covalent bond, —O—, —S—, —NR—, or —C(O)NR—;
Z¹ and Z² are independently N or C, where one of Z¹ and Z² is N and the other of Z and Z² is C;
each [symbol] is independently a single or double bond;
Ring A is a ring selected from phenylenyl, pyridinylenyl,

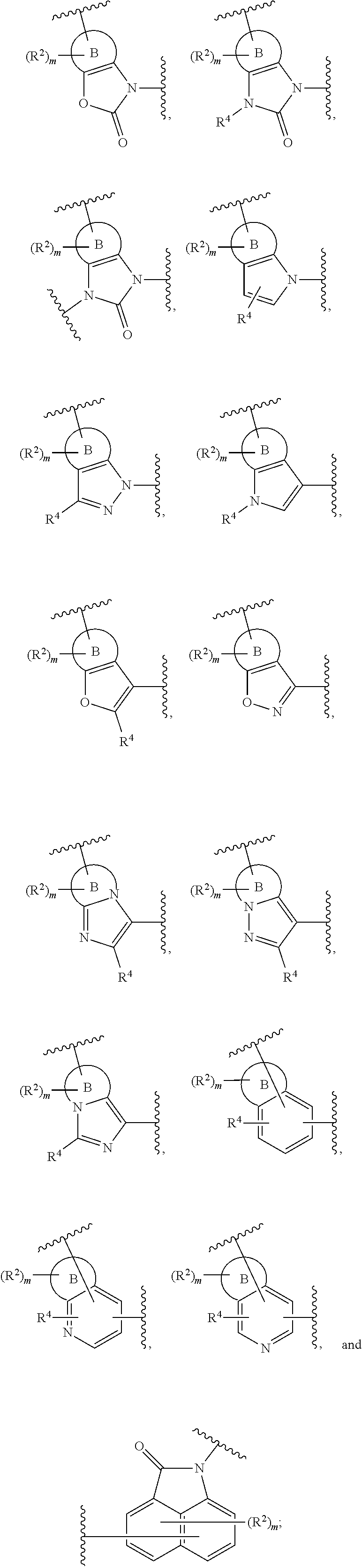

Ring B is a fused ring selected from benzo, a 5-6 membered saturated or partially unsaturated heterocyclyl having 1-3 heteroatoms independently selected from nitrogen, oxygen, and sulfur, or a 5-6 membered heteroaryl containing 1-4 heteroatoms independently selected from nitrogen, oxygen, and sulfur;
Ring C is phenylenyl, pyridylenyl, a 9-10 membered saturated or partially unsaturated bicyclic heterocyclylenyl having 1-3 heteroatoms independently selected from nitrogen, oxygen, and sulfur, 8-10 membered bicyclic heteroarylenyl having 1-3 heteroatoms independently selected from nitrogen, oxygen, and sulfur, 12-15 membered saturated or partially unsaturated tricyclic spirocyclic heterocyclylenyl having 1-3 heteroatoms independently selected from nitrogen, oxygen;
R^{a} is hydrogen, halogen, —CN, —OR, oxo, C₁₋₆alkyl, C₁₋₆haloalkyl, C₃₋₆cycloalkyl, or:
   two R^{a} on the same carbon atom or adjacent carbon atoms connect to form a 3-6 membered saturated carbocyclic or heterocyclic ring having 1-2 heteroatoms selected from nitrogen, oxygen, and sulfur;
a is 0, 1, or 2;
R^{b} is hydrogen, or:
   R^{b} connects with the nitrogen where R¹ is attached to form an optionally substituted 5-6 membered partially unsaturated or aromatic heterocyclic ring having 0-2 heteroatoms in addition to the nitrogen atom where R¹ is attached selected from nitrogen, oxygen, and sulfur;
R¹ is hydrogen, C₁₋₆ alkyl, C₁₋₆ haloalkyl, or C₃₋₆ cycloalkyl;
each R² and R⁵ is independently hydrogen, halogen, —CN, —NO₂, —OR, oxo, —SR, —NR₂, —SiR₃, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, or R^{A};
m and n are independently 0, 1, 2, 3, or 4;
R³ is hydrogen, or a C₁₋₆ alkyl or C₃₋₆ cycloalkyl optionally substituted with 1-2 substituents selected from halogen, —CN, C₁₋₆alkyl, C₁₋₆haloalkyl, and —OR, or:
   R³ and R⁵ are joined by a bivalent, saturated or partially unsaturated, straight or branched Cu hydrocarbon chain, wherein 0-2 methylene units of the chain are independently replaced by —CR₂—, —CRF—, —CF₂—, —O—, —NR—, —C(O)—;
R⁴ is hydrogen, halogen, C₁₋₆ alkyl, C₁₋₆ haloalkyl, C₃₋₆ cycloalkyl, —OC₁₋₆ alkyl, or —OC₁₋₆ haloalkyl;
L is a covalent bond, —CR₂—, —CRF—, —CF₂—, —O—, —NR—, —C(O)—, or a bivalent, saturated or partially unsaturated, straight or branched C₁₋₁₀ hydrocarbon chain, wherein 0-6 methylene units of L are independently replaced by -Cy-, —CR₂—, —CRF—, —CF₂—, —CR(OR)—, —O—, —NR—, —C(O)—; each -Cy- is independently an optionally substituted bivalent ring selected from phenylenyl, 8-10 membered bicyclic arylenyl, 4-7 membered saturated or partially unsaturated carbocyclylenyl, 6-11 membered saturated or partially unsaturated spirocyclic or bridged bicyclic carbocyclylenyl, 8-10 membered bicyclic saturated or partially unsaturated carbocyclylenyl, 4-7 membered saturated or partially unsaturated heterocyclylenyl having 1-2 heteroatoms independently selected from nitrogen, oxygen, and sulfur, 6-11 membered saturated or partially unsaturated spirocyclic or bridged bicyclic heterocyclylenyl having 1-2 heteroatoms independently selected from nitrogen, oxygen, and sulfur, 8-10 membered bicyclic saturated or partially unsaturated heterocyclylenyl having 1-2 heteroatoms independently selected from nitrogen, oxygen, and sulfur, 5-6 membered heteroarylenyl having 1-4 heteroatoms independently selected from nitrogen, oxygen, and sulfur, and 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur; and
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

As described above, in certain embodiments, the present invention provides a compound of formula I-a′:

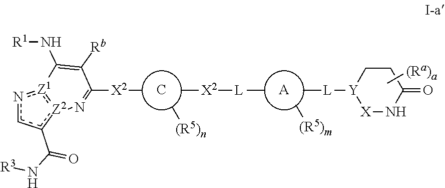

or a pharmaceutically acceptable salt thereof, wherein:
X is a bivalent moiety selected from —CH₂— or —C(O)—;
X¹ is a covalent bond,

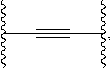 [SMILES: *C#C*]

or an optionally substituted ring selected from 4-6 membered saturated carbocyclylenyl or heterocyclylenyl having 1-2 heteroatoms independently selected from nitrogen, oxygen, and sulfur, phenylenyl, 5-6 membered heteroarylenyl having 1-2 heteroatoms independently selected from nitrogen, oxygen, and sulfur, and a 5-8 membered saturated bridged bicyclic carbocyclylenyl or heterocyclylenyl having 1-2 heteroatoms independently selected from nitrogen, oxygen, and sulfur;
X² is a covalent bond, —O—, or —NR—;
Y is N or CH;
Y¹ is a covalent bond, —O—, —S—, —NR—, or —C(O)NR—;
Z¹ and Z² are independently N or C, where one of Z¹ and Z² is N and the other of Z¹ and Z² is C;
each [symbol] is independently a single or double bond;
Ring A is a ring selected from phenylenyl, pyridinylenyl,

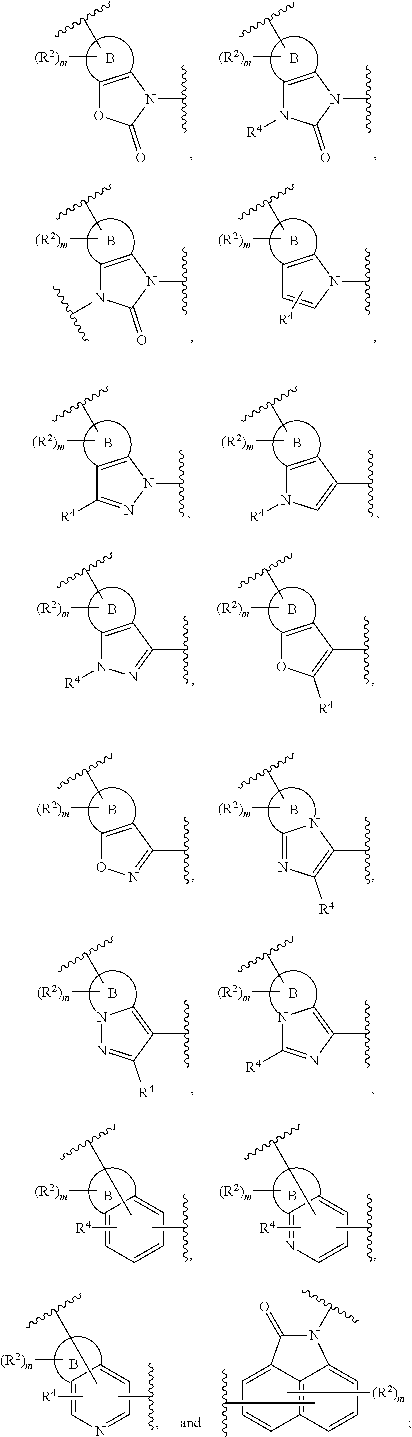

Ring B is a fused ring selected from benzo, a 5-6 membered saturated or partially unsaturated heterocyclyl having 1-3 heteroatoms independently selected from nitrogen, oxygen, and sulfur, or a 5-6 membered heteroaryl containing 1-4 heteroatoms independently selected from nitrogen, oxygen, and sulfur;
Ring C is phenylenyl, pyridylenyl, a 9-10 membered saturated or partially unsaturated bicyclic heterocyclylenyl having 1-3 heteroatoms independently selected from nitrogen, oxygen, and sulfur, 8-10 membered bicyclic heteroarylenyl having 1-3 heteroatoms independently selected from nitrogen, oxygen, and sulfur, 12-15 membered saturated or partially unsaturated tricyclic spirocyclic heterocyclylenyl having 1-3 heteroatoms independently selected from nitrogen, oxygen;
R^{a} is hydrogen, halogen, —CN, —OR, oxo, C₁₋₆alkyl, C₁₋₆haloalkyl, C₃₋₆cycloalkyl, or:
   two R^{a} on the same carbon atom or adjacent carbon atoms connect to form a 3-6 membered saturated carbocyclic or heterocyclic ring having 1-2 heteroatoms selected from nitrogen, oxygen, and sulfur;
a is 0, 1, or 2;
R^{b} is hydrogen, or:
   R^{b} connects with the nitrogen where R¹ is attached to form an optionally substituted 5-6 membered partially unsaturated or aromatic heterocyclic ring having 0-2 heteroatoms in addition to the nitrogen atom where R¹ is attached selected from nitrogen, oxygen, and sulfur;
R¹ is hydrogen, C₁₋₆ alkyl, C₁₋₆ haloalkyl, or C₃₋₆ cycloalkyl;
each R² and R⁵ is independently hydrogen, halogen, —CN, —NO₂, —OR, oxo, —SR, —NR₂, —SiR₃, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, or R^{A};
m and n are independently 0, 1, 2, 3, or 4;
R³ is hydrogen, or a C₁₋₆ alkyl or 3-9 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur optionally substituted with 1-2 substituents selected from halogen, —CN, C₁₋₆ alkyl, C₁₋₆ haloalkyl, —OR, —CH₂OR, or a 5-membered heteroaryl having 1-3 heteroatoms independently selected from nitrogen, oxygen, and sulfur, or:
   R³ and R⁵ are joined by a bivalent, saturated or partially unsaturated, straight or branched C₃₋₆ hydrocarbon chain, wherein 0-2 methylene units of the chain are independently replaced by —CR₂—, —CRF—, —CF₂—, —O—, —NR—, —C(O)—;
R⁴ is hydrogen, halogen, C₁₋₆ alkyl, C₁₋₆ haloalkyl, C₃₋₆ cycloalkyl, —OC₁₋₆ alkyl, or —OC₁₋₆ haloalkyl;
L is a covalent bond, —CR₂—, —CRF—, —CF₂—, —O—, —NR—, —C(O)—, or a bivalent, saturated or partially unsaturated, straight or branched C₁₋₁₀ hydrocarbon chain, wherein 0-6 methylene units of L are independently replaced by -Cy-, —CR₂—, —CRF—, —CF₂—, —CR(OR)—, —O—, —NR—, —C(O)—;
each -Cy- is independently an optionally substituted bivalent ring selected from phenylenyl, 8-10 membered bicyclic arylenyl, 4-7 membered saturated or partially unsaturated carbocyclylenyl, 6-11 membered saturated or partially unsaturated spirocyclic or bridged bicyclic carbocyclylenyl, 8-10 membered bicyclic saturated or partially unsaturated carbocyclylenyl, 4-7 membered saturated or partially unsaturated heterocyclylenyl having 1-2 heteroatoms independently selected from nitrogen, oxygen, and sulfur, 6-11 membered saturated or partially unsaturated spirocyclic or bridged bicyclic heterocyclylenyl having 1-2 heteroatoms independently selected from nitrogen, oxygen, and sulfur, 8-10 membered bicyclic saturated or partially unsaturated heterocyclylenyl having 1-2 heteroatoms independently selected from nitrogen, oxygen, and sulfur, 5-6 membered heteroarylenyl having 1-4 heteroatoms independently selected from nitrogen, oxygen, and sulfur, and 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur; and
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

As described above, in certain embodiments, the present invention provides a compound of formula I-a″:

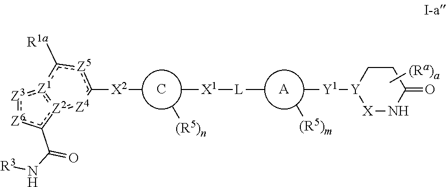

or a pharmaceutically acceptable salt thereof, wherein:
X is a bivalent moiety selected from —CH₂— or C(O)—;
X¹ is a covalent bond,

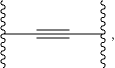 [SMILES: *C#C*]

or an optionally substituted ring selected from phenylenyl, 4-11 membered saturated or partially unsaturated monocyclic, bicyclic, bridged bicyclic, or spirocyclic carbocyclylenyl or heterocyclylenyl having 1-2 heteroatoms independently selected from nitrogen, oxygen, and sulfur, and 5-6 membered heteroarylenyl having 1-2 heteroatoms independently selected from nitrogen, oxygen, and sulfur;
X² is a covalent bond, —CR₂—, —O—, or —NR—;
Y is N or CH;
Y¹ is a covalent bond, —O—, —S—, —NR—, or —C(O)NR—;
Z¹, Z², Z⁴, and Z⁶ are independently N or C, where one of Z¹ and Z² is N and the other of Z and Z² is C;
Z³ is N, O, or S;
Z⁵ is N or CR^{b};
each [symbol] is independently a single or double bond;
Ring A is phenylenyl, naphthalenyl, pyridinylenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur;
Ring C is phenylenyl, pyridylenyl, an 8-15 membered saturated or partially unsaturated monocyclic, bicyclic, bridged bicyclic, spirocyclic, or tricyclic carbocyclylenyl or heterocyclylenyl having 1-3 heteroatoms independently selected from nitrogen, oxygen, and sulfur, or an 8-10 membered bicyclic heteroarylenyl having 1-3 heteroatoms independently selected from nitrogen, oxygen, and sulfur;
R^{a} is hydrogen, halogen, —CN, —OR, oxo, C₁₋₆alkyl, C₁₋₆haloalkyl, C₃₋₆ cycloalkyl, or:
   two R^{a} on the same carbon atom or adjacent carbon atoms connect to form a 3-6 membered saturated carbocyclic or heterocyclic ring having 1-2 heteroatoms selected from nitrogen, oxygen, and sulfur;
a is 0, 1, or 2;
R^{b} is hydrogen, or:
   R^{b} connects with the nitrogen where R¹ is attached to form an optionally substituted 5-6 membered partially unsaturated or aromatic heterocyclic ring having 0-2 heteroatoms in addition to the nitrogen atom where R¹ is attached selected from nitrogen, oxygen, and sulfur;
R^{1a} is R¹, NHR¹, OR¹, or SR¹;
R¹ is hydrogen, C₁₋₆ alkyl, C₁₋₆ haloalkyl, C₃₋₆ cycloalkyl, C₃₋₆ heterocycloalkyl, or C₁₋₆ alkyl-O—C₁₋₆ alkyl;
each R² and R⁵ is independently hydrogen, halogen, —CN, —NO₂, —OR, oxo, —SR, —NR₂, —SiR₃, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, or R^{A}; or:
   two R² groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur;
m and n are independently 0, 1, 2, 3, or 4;
R³ is hydrogen, or an optionally substituted group selected from C₁₋₆ aliphatic, phenyl, a 3-11 membered saturated or partially unsaturated monocyclic, bicyclic, bridged bicyclic, or spirocyclic carbocyclylenyl or heterocyclylenyl having 1-3 heteroatoms independently selected from nitrogen, oxygen, and sulfur, and a 5-10 membered monocyclic or bicyclic heteroaryl having 1-3 heteroatoms independently selected from nitrogen, oxygen, and sulfur, or:
   R³ and R⁵ are joined by a bivalent, saturated or partially unsaturated, straight or branched C₃₋₆ hydrocarbon chain, wherein 0-2 methylene units of the chain are independently replaced by —CR₂—, —CRF—, —CF₂—, —O—, —NR—, —C(O)—;
R⁴ is hydrogen, halogen, C₁₋₆ alkyl, C₁₋₆ haloalkyl, C₃₋₆ cycloalkyl, —OC₁₋₆ alkyl, or —OC₁₋₆haloalkyl; L is a covalent bond, —CR₂—, —CRF—, —CF₂—, —O—, —NR—, —C(O)—, or a bivalent, saturated or partially unsaturated, straight or branched C₁₋₁₀ hydrocarbon chain, wherein 0-6 methylene units of L are independently replaced by -Cy-, —CR₂—, —CRF—, —CF₂—, —CR(OR)—, —O—, —NR—, —C(O)—, or S(O)₂;
each -Cy- is independently an optionally substituted bivalent ring selected from phenylenyl, 8-10 membered bicyclic arylenyl, 4-7 membered saturated or partially unsaturated carbocyclylenyl, 6-11 membered saturated or partially unsaturated spirocyclic or bridged bicyclic carbocyclylenyl, 8-10 membered bicyclic saturated or partially unsaturated carbocyclylenyl, 4-7 membered saturated or partially unsaturated heterocyclylenyl having 1-2 heteroatoms independently selected from nitrogen, oxygen, and sulfur, 6-11 membered saturated or partially unsaturated spirocyclic or bridged bicyclic heterocyclylenyl having 1-2 heteroatoms independently selected from nitrogen, oxygen, and sulfur, 8-10 membered bicyclic saturated or partially unsaturated heterocyclylenyl having 1-2 heteroatoms independently selected from nitrogen, oxygen, and sulfur, 5-6 membered heteroarylenyl having 1-4 heteroatoms independently selected from nitrogen, oxygen, and sulfur, and 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur; and
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In certain embodiments, the present invention provides a compound of formula I-b:

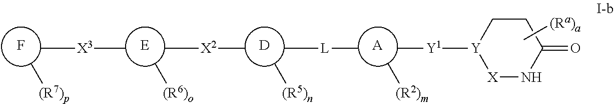

or a pharmaceutically acceptable salt thereof, wherein:
X is a bivalent moiety selected from —CH₂— or —C(O)—;
X² and X³ are independently a covalent bond, —O—, or —NR—;
Y is N or CH;
Y¹ is a covalent bond, —O—, —S—, —NR—, or —C(O)NR—;

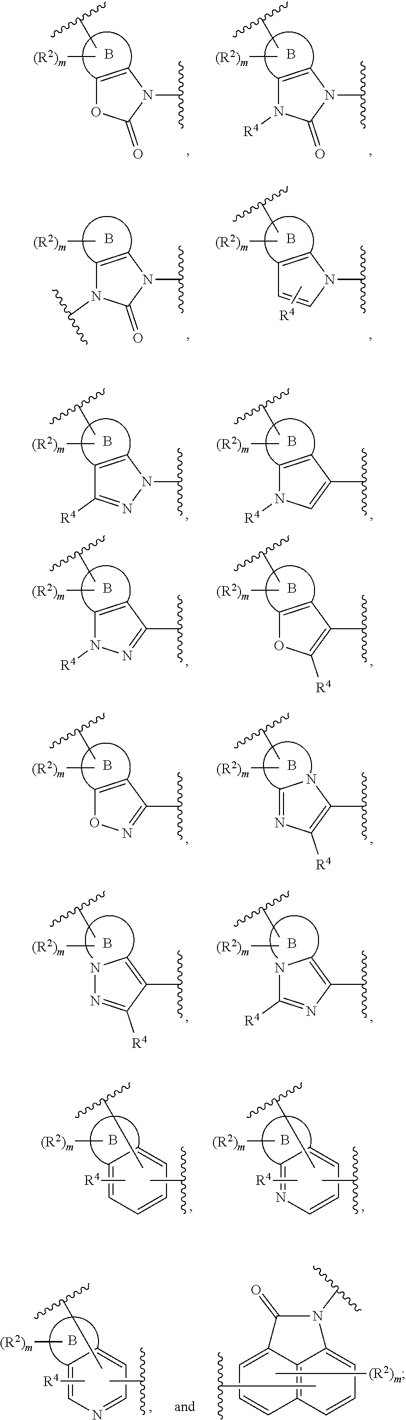

Ring B is a fused ring selected from benzo, a 5-6 membered saturated or partially unsaturated heterocyclyl having 1-3 heteroatoms independently selected from nitrogen, oxygen, and sulfur, or a 5-6 membered heteroaryl containing 1-4 heteroatoms independently selected from nitrogen, oxygen, and sulfur;
Ring D is a bivalent ring selected from phenylenyl, 4-7 membered saturated or partially unsaturated carbocyclylenyl or heterocyclylenyl having 1-2 heteroatoms independently selected from nitrogen, oxygen, and sulfur, and a 5-6 membered heteroarylenyl ring having 1-4 heteroatoms independently selected from nitrogen, oxygen, and sulfur;
Ring E is a bivalent ring selected from phenylenyl, naphthylenyl, 4-7 membered saturated or partially unsaturated carbocyclylenyl or heterocyclylenyl having 1-2 heteroatoms independently selected from nitrogen, oxygen, and sulfur, 5-6 membered heteroarylenyl ring having 1-4 heteroatoms independently selected from nitrogen, oxygen, and sulfur, a 9-10 membered saturated or partially unsaturated bicyclic heterocyclylenyl having 1-4 heteroatoms independently selected from nitrogen, oxygen, and sulfur, and 8-10 membered bicyclic heteroarylenyl having 1-4 heteroatoms independently selected from nitrogen, oxygen, and sulfur;
Ring F is phenyl, 4-7 membered saturated or partially unsaturated carbocyclyl or heterocyclyl having 1-2 heteroatoms independently selected from nitrogen, oxygen, and sulfur, and a 5-6 membered heteroaryl ring having 1-4 heteroatoms independently selected from nitrogen, oxygen, and sulfur;
R^{a} is hydrogen, halogen, —CN, —OR, oxo, C₁₋₆alkyl, C₁₋₆haloalkyl, C₃₋₆cycloalkyl, or:
   two R^{a} on the same carbon atom or adjacent carbon atoms connect to form a 3-6 membered saturated carbocyclic or heterocyclic ring having 1-2 heteroatoms selected from nitrogen, oxygen, and sulfur;
a is 0, 1, or 2;
each R², R⁵, R⁶, and R⁷ are independently hydrogen, halogen, —CN, —NO₂, —OR, oxo, —SR, —NR₂, —SiR₃, —S(O)₂R, —S(O)₂NR₂, —S(O)R, —C(O)R. —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, or R^{A};
each of m, n, o, and p are independently 0, 1, 2, 3, or 4;
R⁴ is hydrogen, halogen, C₁₋₆ alkyl, C₁₋₆ haloalkyl, C₃₋₆ cycloalkyl, —OC₁₋₆ alkyl, or —OC₁₋₆ haloalkyl;
L is a covalent bond, —CR₂—, —CRF—, —CF₂—, —O—, —NR—, —C(O)—, or a bivalent, saturated or partially unsaturated, straight or branched C₁₋₁₀ hydrocarbon chain, wherein 0-6 methylene units of L are independently replaced by -Cy-, —CR₂—, —CRF—, —CF₂—, —CR(OR)—, —O—, —NR—, —C(O)—;
each -Cy- is independently an optionally substituted bivalent ring selected from phenylenyl, 8-10 membered bicyclic arylenyl, 4-7 membered saturated or partially unsaturated carbocyclylenyl, 6-11 membered saturated or partially unsaturated spirocyclic or bridged bicyclic carbocyclylenyl, 8-10 membered bicyclic saturated or partially unsaturated carbocyclylenyl, 4-7 membered saturated or partially unsaturated heterocyclylenyl having 1-2 heteroatoms independently selected from nitrogen, oxygen, and sulfur, 6-11 membered saturated or partially unsaturated spirocyclic or bridged bicyclic heterocyclylenyl having 1-2 heteroatoms independently selected from nitrogen, oxygen, and sulfur, 8-10 membered bicyclic saturated or partially unsaturated heterocyclylenyl having 1-2 heteroatoms independently selected from nitrogen, oxygen, and sulfur, 5-6 membered heteroarylenyl having 1-4 heteroatoms independently selected from nitrogen, oxygen, and sulfur, and 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur; and
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In certain embodiments, the present invention provides a compound of formula I-c:

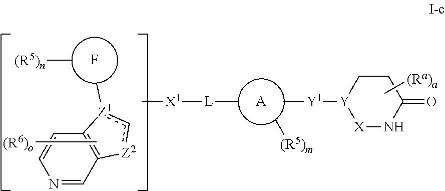

or a pharmaceutically acceptable salt thereof, wherein:
X is a bivalent moiety selected from —CH₂— or —C(O)—;
X¹ is a covalent bond,

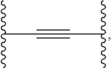 [SMILES: *C#C*]

or an optionally substituted ring selected from 4-6 membered saturated carbocyclylenyl or heterocyclylenyl having 1-2 heteroatoms independently selected from nitrogen, oxygen, and sulfur, phenylenyl, 5-6 membered heteroarylenyl having 1-2 heteroatoms independently selected from nitrogen, oxygen, and sulfur, and a 5-8 membered saturated bridged bicyclic heterocyclylenyl having 1-2 heteroatoms independently selected from nitrogen, oxygen, and sulfur;
Y is N or CH;
Y¹ is a covalent bond, —O—, —S—, —NR—, or —C(O)NR—;
Z¹ and Z² are independently N or C, where one of Z¹ and Z² is N and the other of Z¹ and Z² is C;
each [symbol] is independently a single or double bond;
Ring A is a ring selected from phenylenyl, pyridinylenyl,

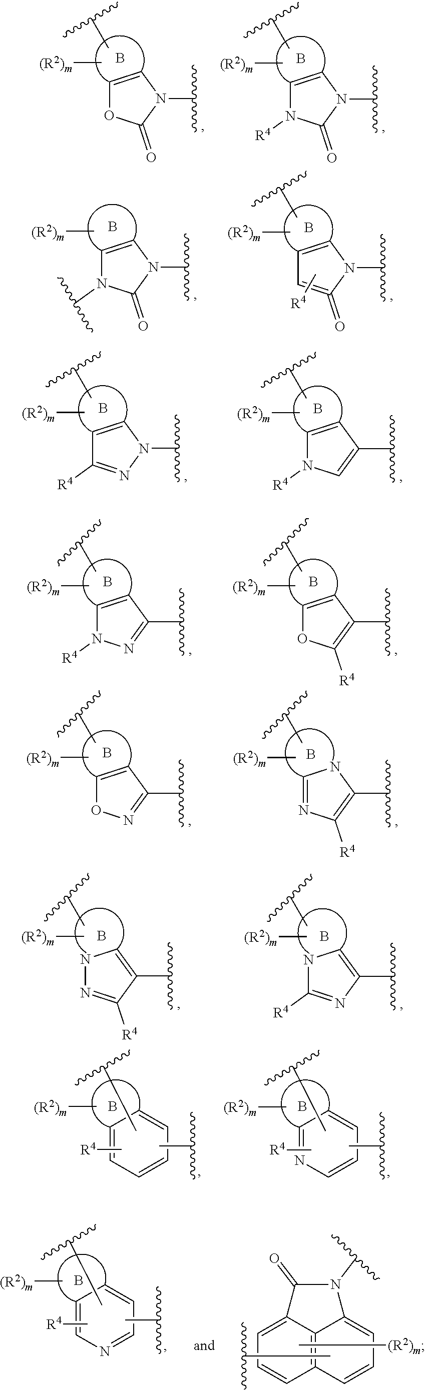

Ring B is a fused ring selected from benzo, a 5-6 membered saturated or partially unsaturated heterocyclyl having 1-3 heteroatoms independently selected from nitrogen, oxygen, and sulfur, or a 5-6 membered heteroaryl containing 1-4 heteroatoms independently selected from nitrogen, oxygen, and sulfur;
Ring F is phenyl, 4-7 membered saturated or partially unsaturated carbocyclyl or heterocyclyl having 1-2 heteroatoms independently selected from nitrogen, oxygen, and sulfur, and a 5-6 membered heteroaryl ring having 1-4 heteroatoms independently selected from nitrogen, oxygen, and sulfur;
R^{a} is hydrogen, halogen, —CN, —OR, oxo, C₁₋₆alkyl, C₁₋₆haloalkyl, C₃₋₆cycloalkyl, or:
   two R^{a} on the same carbon atom or adjacent carbon atoms connect to form a 3-6 membered saturated carbocyclic or heterocyclic ring having 1-2 heteroatoms selected from nitrogen, oxygen, and sulfur;
a is 0, 1, or 2;
each R², R⁵, and R⁶ are independently hydrogen, halogen, —CN, —NO₂, —OR, oxo, —SR, —NR₂, —SiR₃, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, or R^{A}, or:
   two R⁶ on adjacent atoms connect to form a fused optionally substituted 5-6 membered partially saturated or aromatic heterocyclic ring having 1-2 heteroatoms selected from nitrogen, oxygen, and sulfur;
each of m, n, and o are independently 0, 1, 2, 3, or 4;
R⁴ is hydrogen, halogen, C₁₋₆ alkyl, C₁₋₆ haloalkyl, C₃₋₆ cycloalkyl, —OC₁₋₆ alkyl, or —OC₁₋₆ haloalkyl;
L is a covalent bond, —CR₂—, —CRF—, —CF₂—, —O—, —NR—, —C(O)—, or a bivalent, saturated or partially unsaturated, straight or branched C₁₋₁₀ hydrocarbon chain, wherein 0-6 methylene units of L are independently replaced by -Cy-, —CR₂—, —CRF—, —CF₂—, —CR(OR)—, —O—, —NR—, —C(O)—;
each -Cy- is independently an optionally substituted bivalent ring selected from phenylenyl, 8-10 membered bicyclic arylenyl, 4-7 membered saturated or partially unsaturated carbocyclylenyl, 6-11 membered saturated or partially unsaturated spirocyclic or bridged bicyclic carbocyclylenyl, 8-10 membered bicyclic saturated or partially unsaturated carbocyclylenyl, 4-7 membered saturated or partially unsaturated heterocyclylenyl having 1-2 heteroatoms independently selected from nitrogen, oxygen, and sulfur, 6-11 membered saturated or partially unsaturated spirocyclic or bridged bicyclic heterocyclylenyl having 1-2 heteroatoms independently selected from nitrogen, oxygen, and sulfur, 8-10 membered bicyclic saturated or partially unsaturated heterocyclylenyl having 1-2 heteroatoms independently selected from nitrogen, oxygen, and sulfur, 5-6 membered heteroarylenyl having 1-4 heteroatoms independently selected from nitrogen, oxygen, and sulfur, and 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur; and
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

As defined herein and described above, X is a bivalent moiety selected from —CH— or —C(O)—.

In some embodiments, X is —CH₂—. In some embodiments, X is —C(O)—.

In some embodiments, X is as depicted in the compounds of Table 1, below.

As defined herein and described above, X¹ is a covalent bond,

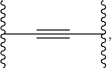 [SMILES: *C#C*]

or an optionally substituted ring selected from phenylenyl, 4-11 membered saturated or partially unsaturated monocyclic, bicyclic, bridged bicyclic, or spirocyclic carbocyclylenyl or heterocyclylenyl having 1-2 heteroatoms independently selected from nitrogen, oxygen, and sulfur, and 5-6 membered heteroarylenyl having 1-2 heteroatoms independently selected from nitrogen, oxygen, and sulfur.

In some embodiments, X¹ is an optionally substituted 5-11 membered saturated or partially unsaturated monocyclic, bicyclic, bridged bicyclic, or spirocyclic carbocyclylenyl. In some embodiments, X¹ is an optionally substituted 5-11 membered saturated or partially unsaturated monocyclic, bicyclic, bridged bicyclic, or spirocyclic heterocyclylenyl having 1-2 heteroatoms independently selected from nitrogen, oxygen, and sulfur. In some embodiments, X¹ is an optionally substituted 5-6 membered heteroarylenyl having 1-2 heteroatoms independently selected from nitrogen, oxygen, and sulfur.

In some embodiments, X¹ is an optionally substituted 5-8 membered saturated monocyclic or bridged bicyclic carbocyclylenyl. In some embodiments, X¹ is an optionally substituted 5-6 membered saturated monocyclic carbocyclylenyl. In some embodiments, X¹ is an optionally substituted 7-8 membered saturated bridged bicyclic carbocyclylenyl.

In some embodiments, X¹ is an optionally substituted 5-8 membered saturated monocyclic or bridged bicyclic heterocyclylenyl having 1-2 heteroatoms independently selected from nitrogen and oxygen. In some embodiments, X¹ is an optionally substituted 5-6 membered saturated monocyclic heterocyclylenyl having 1-2 heteroatoms independently selected from nitrogen and oxygen. In some embodiments, X¹ is an optionally substituted 6-8 membered saturated bridged bicyclic heterocyclylenyl having 1-2 heteroatoms independently selected from nitrogen and oxygen.

In some embodiments, X¹ is an optionally substituted 9-11 membered saturated spirocyclic heterocyclylenyl having 1-2 heteroatoms independently selected from nitrogen and oxygen. In some embodiments, X¹ is an optionally substituted 9-11 membered saturated spirocyclic heterocyclylenyl having 2 nitrogen.

As defined herein and described above, X¹ is a covalent bond,

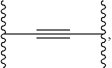 [SMILES: *C#C*]

or an optionally substituted ring selected from 4-6 membered saturated carbocyclylenyl or heterocyclylenyl having 1-2 heteroatoms independently selected from nitrogen, oxygen, and sulfur, phenylenyl, 5-6 membered heteroarylenyl having 1-2 heteroatoms independently selected from nitrogen, oxygen, and sulfur, and a 5-8 membered saturated bridged bicyclic carbocyclylenyl or heterocyclylenyl having 1-2 heteroatoms independently selected from nitrogen, oxygen, and sulfur.

In some embodiments, X¹ is a covalent bond. In some embodiments, X¹ is

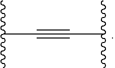 [SMILES: *C#C*]

In some embodiments, X¹ is an optionally substituted 4-6 membered saturated carbocyclylenyl. In some embodiments, X¹ an optionally substituted 4-6 membered saturated heterocyclylenyl having 1-2 heteroatoms independently selected from nitrogen, oxygen, and sulfur. In some embodiments, X¹ is an optionally substituted phenylenyl. In some embodiments, X¹ is an optionally substituted 5-6 membered heteroarylenyl having 1-2 heteroatoms independently selected from nitrogen, oxygen, and sulfur. In some embodiments, X¹ an optionally substituted 5-8 membered saturated bridged bicyclic carbocyclylenyl. In some embodiments, X¹ an optionally substituted 5-8 membered saturated bridged bicyclic heterocyclylenyl having 1-2 heteroatoms independently selected from nitrogen, oxygen, and sulfur.

In some embodiments, X¹ is an optionally substituted 6-membered heteroarylenyl having 1-2 heteroatoms independently selected from nitrogen, oxygen, and sulfur. In some embodiments, X¹ an optionally substituted 8-membered saturated bridged bicyclic heterocyclylenyl having 1-2 heteroatoms independently selected from nitrogen, oxygen, and sulfur.

In some embodiments, X¹ is unsubstituted. In some embodiments, X¹ is substituted. In some embodiments, X¹ is substituted with halogen, C₁₋₆ alkyl, C₁₋₆ haloalkyl, C₃₋₆ cycloalkyl, —OC₁₋₆ alkyl, or —OC₁₋₆ haloalkyl. In some embodiments, X¹ is substituted with fluoro, chloro, or methyl.

In some embodiments, X¹ is

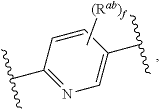

wherein each R^{ab} is independently halogen or C₁₋₃ alkyl; and f is an integer from 0 to 2. In some embodiments, each R^{ab} is halogen. In some embodiments, each R^{ab} is fluoro. In some embodiments, f is 1. In some embodiments, f is 2. In some embodiments, f is 0.

In some embodiments, X¹ is

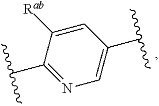

wherein R^{ab} is halogen or C₁₋₃ alkyl. In some embodiments, R^{ab} is halogen. In some embodiments, R^{ab} is fluoro.

In some embodiments, X¹ is 5-6 membered heterocyclylenyl having one or two ring nitrogens, 4-6 membered saturated carbocyclylenyl, 5-6 membered heteroarylenyl having one or two ring nitrogens, or 5-8 membered saturated bridged bicyclic heterocyclylenyl having one or two ring nitrogens, wherein each 5 or 6-membered heterocyclylenyl, 4-6 membered saturated carbocyclylenyl, 5 or 6-membered heteroarylenyl, and 5-8 membered saturated bridged bicyclic heterocyclylenyl, is optionally substituted with one or two occurrences of halogen.

In some embodiments, X¹ is 5-6 membered heterocyclylenyl having one or two ring nitrogens, wherein each 5 or 6-membered heterocyclylenyl is optionally substituted with one or two occurrences of halogen. In some embodiments, X¹ is 5-6 membered heteroarylenyl having one or two ring nitrogens, wherein each 5 or 6-membered heteroarylenyl is optionally substituted with one or two occurrences of halogen.

In some embodiments, X¹ is a covalent bond,

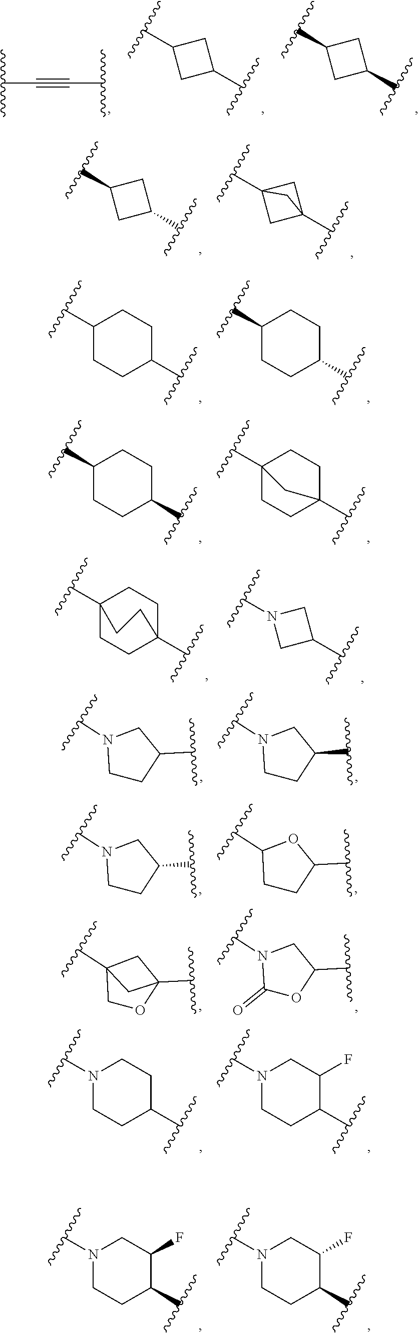

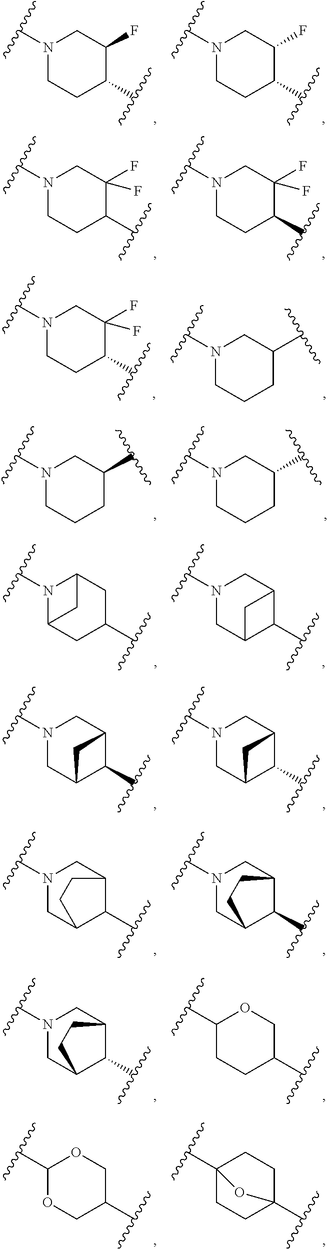

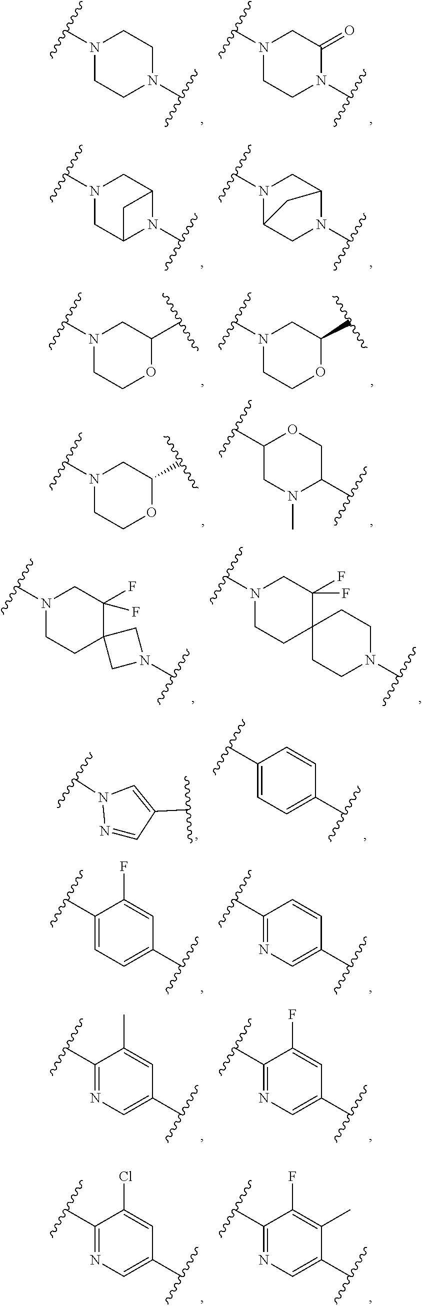

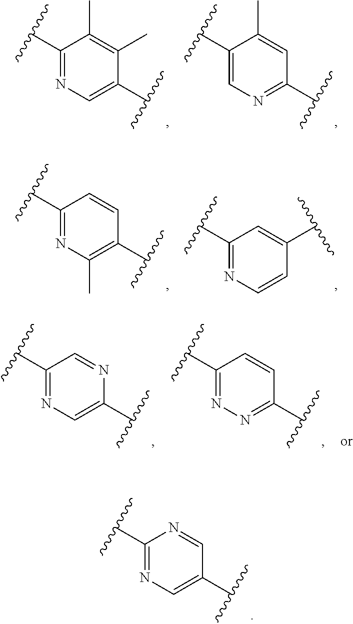

In some embodiments, X¹ is as depicted in the compounds of Table 1, below.

As defined herein and described above, X² is a covalent bond, —CR₂—, —O—, or —NR—.

In some embodiments, X² is a covalent bond. In some embodiments, X² is —CR₂—. In some embodiments, X² is —O—. In some embodiments, X² is —NR—.

In some embodiments, X² is —NH—. In some embodiments, X² is —CH₂—.

As defined herein and described above, X³ is a covalent bond, —O—, or —NR—.

In some embodiments, X³ is a covalent bond. In some embodiments, X³ is —O—. In some embodiments, X³ is —NR—.

In some embodiments, X³ is —NH—.

In some embodiments, X² is —NH— and X³ is —NH—. In some embodiments, X² is —NH— and X³ is a covalent bond.

In some embodiments, X² and X³ are as depicted in the compounds of Table 1, below.

As defined herein and described above, Y is N or CH.

In some embodiments, Y is N. In some embodiments, Y is CH.

In some embodiments, Y is as depicted in the compounds of Table 1, below.

As defined herein and described above, Y¹ is a covalent bond, —O—, —S—, —NR—, or —C(O)NR—.

In some embodiments, Y¹ is a covalent bond. In some embodiments, Y¹ is —O—. In some embodiments, Y¹ is —S—. In some embodiments, Y¹ is —NR—. In some embodiments, Y¹ is —C(O)NR—.

In some embodiments, Y¹ is —NH—. In some embodiments, Y¹ is —C(O)NH—.

In some embodiments, Y¹ is a covalent bond, —O—, —NR—, —S—, —CR₂—, —NRC(O)—, or —C(O)NR—. In some embodiments, Y¹ is —O—, —NR—, —S—, —CR₂—, —NRC(O)—, or —C(O)NR—. In some embodiments, Y¹ is —CR₂—. In some embodiments, Y¹ is —CH₂—. In some embodiments, Y¹ is —NHC(O)—.

In some embodiments, Y¹ is as depicted in the compounds of Table 1, below.

As defined herein and described above, R^{a} is hydrogen, halogen, —CN, —OR, oxo. C₁₋₆ alkyl, C₁₋₆ haloalkyl, C₃₋₆ cycloalkyl, or two R on the same carbon atom or adjacent carbon atoms connect to form a 3-6 membered saturated carbocyclic or heterocyclic ring having 1-2 heteroatoms selected from nitrogen, oxygen, and sulfur.

In some embodiments, R^{a} is hydrogen. In some embodiments, R^{a} is halogen. In some embodiments, R^{a} is —CN. In some embodiments, R^{a} is —OR. In some embodiments, R^{a} is oxo. In some embodiments, R^{a} is C₁₋₆ alkyl. In some embodiments, R^{a} is C₁₋₆ haloalkyl. In some embodiments, R^{a} is C₃-6 cycloalkyl. In some embodiments, two R^{a} on the same carbon atom or adjacent carbon atoms connect to form a 3-6 membered saturated carbocyclic or heterocyclic ring having 1-2 heteroatoms selected from nitrogen, oxygen, and sulfur. In some embodiment two R^{a} on the same carbon atom form geminal dimethyl. In some embodiment two R^{a} on the same carbon atom form geminal difluoro. In some embodiment two R^{a} on the same carbon atom form cyclopropylenyl. In some embodiment two R^{a} on the adjacent carbon atoms form fused cyclopropyl. In some embodiment two R^{a} on the adjacent carbon atoms form a bridged methylene (i.e., —CH₂—). In some embodiment two R^{a} on the adjacent carbon atoms form a bridged ethylene (i.e., —CH₂CH₂—).

In some embodiments, R^{a} is as depicted in the compounds of Table 1, below.

As defined herein and described above, a is 0, 1, or 2.

In some embodiments, a is 0. In some embodiments, a is 1. In some embodiments, a is 2.

In some embodiments, a is as depicted in the compounds of Table 1, below.

In some embodiments,

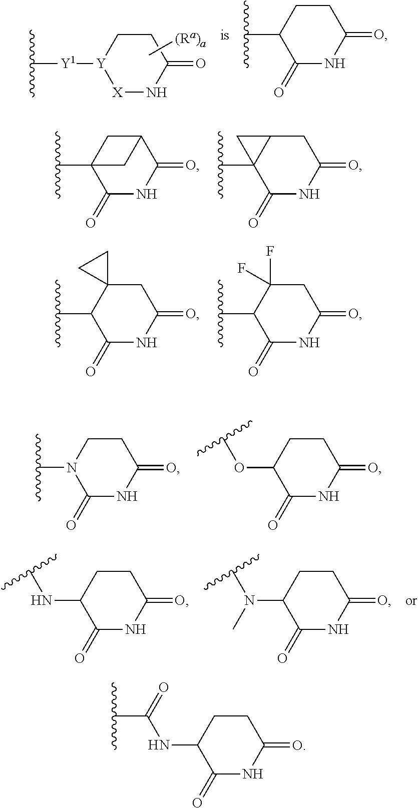

As defined herein and described above, Z¹, Z², Z⁴, and Z⁶ are independently N or C, where one of Z¹ and Z² is N and the other of Z¹ and Z² is C.

As defined herein and described above, Z¹ and Z² are independently N or C, where one of Z¹ and Z² is N and the other of Z¹ and Z² is C.

In some embodiments, Z¹ is N. In some embodiments, Z¹ is C. In some embodiments, Z² is N. In some embodiments, Z² is C.

In some embodiments, Z¹ is C and Z² in N. In some embodiments, Z¹ is N and Z² in C.

In some embodiments, Z⁴ is N. In some embodiments, Z⁴ is C.

In some embodiments, Z⁶ is N. In some embodiments, Z⁶ is C.

As defined herein and described above, Z³ is N, O, or S.

In some embodiments, Z³ is N. In some embodiments, Z³ is 0. In some embodiments, Z³ is S.

As defined herein and described above, Z⁵ is N or CR^{b}.

In some embodiments, Z⁵ is N. In some embodiments, Z⁵ is CR^{b}.

In some embodiments,

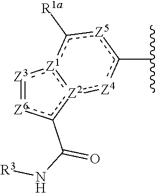

is

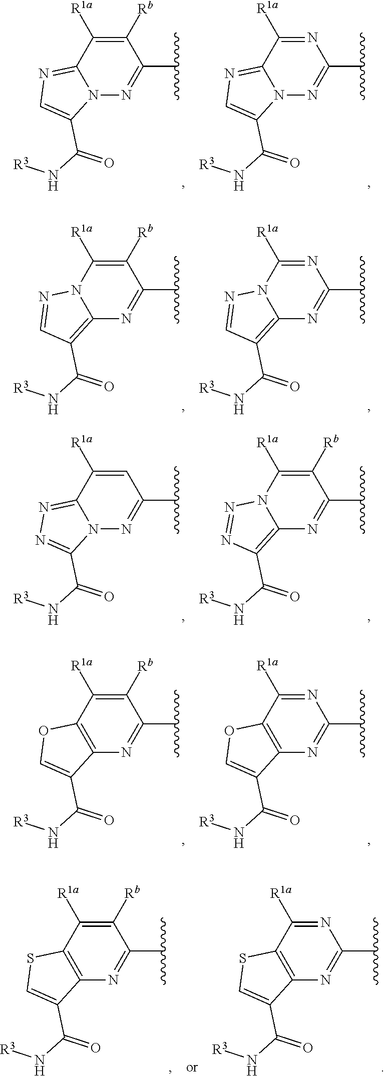

In some embodiments, Z¹, Z², Z³, Z⁴, Z⁵, and Z⁶ are as depicted in the compounds of Table 1, below.

As defined herein and described above, each [symbol] is independently a single or double bond.

In some embodiments, [symbol] is a single bond. In some embodiments, [symbol] is a double bond.

In some embodiments, [symbol] is as depicted in the compounds of Table 1, below.

As defined above and described herein, Rung A is phenylenyl, naphthalenyl, pyridinylenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur.

In some embodiments, Ring A is a phenylenyl. In some embodiments, Ring A is a naphthalenyl. In some embodiments, Ring A is pyridinylenyl. In some embodiments, Ring A is a 4-7 membered saturated or partially unsaturated carbocyclylenyl. In some embodiments, Ring A is a 4-7 membered saturated or partially unsaturated heterocyclylenyl having 1-3 heteroatoms independently selected from nitrogen, oxygen, and sulfur. In some embodiments, Ring A is an 8-10 membered bicyclic saturated or partially unsaturated heterocyclylenyl having 1-2 heteroatoms independently selected from nitrogen, oxygen, and sulfur. In some embodiments, Ring A is a 5-6 membered heteroarylenyl having 1-4 heteroatoms independently selected from nitrogen, oxygen, and sulfur. In some embodiments, Ring A is an 8-10 membered bicyclic heteroarylenyl having 1-5 heteroatoms independently selected from nitrogen, oxygen, and sulfur. In some embodiments, Ring A is an 8-15 membered saturated or partially unsaturated tricyclic heterocyclylenyl having 1-5 heteroatoms independently selected from nitrogen, oxygen, and sulfur. In some embodiments, Ring A is or an 8-15 membered tricyclic heteroarylenyl having 1-5 heteroatoms independently selected from nitrogen, oxygen, and sulfur.

In some embodiments, Ring A is a 9-10 membered saturated or partially unsaturated monocyclic or bicyclic heterocyclylenyl or heteroarylenyl containing 1-4 heteroatoms independently selected from nitrogen, oxygen, and sulfur.

In some embodiments, Ring A is a 8-10 membered saturated or partially unsaturated bicyclic heterocyclylenyl or heteroarylenyl containing 1-4 heteroatoms independently selected from nitrogen, oxygen, and sulfur. In some embodiments, Ring A is a 9-10 membered saturated or partially unsaturated bicyclic heterocyclylenyl or heteroarylenyl containing 1-4 heteroatoms independently selected from nitrogen, oxygen, and sulfur.

In some embodiments, Ring A is a 9-membered saturated or partially unsaturated bicyclic heterocyclylenyl or heteroarylenyl containing 1-4 heteroatoms independently selected from nitrogen, oxygen, and sulfur. In some embodiments, Ring A is a 9-membered saturated or partially unsaturated bicyclic heterocyclylenyl or heteroarylenyl containing 1 nitrogen and 1 oxygen heteroatom.

In some embodiments, Ring A is a 5,6-fused saturated or partially unsaturated bicyclic heterocyclylenyl or heteroarylenyl containing 1-4 heteroatoms independently selected from nitrogen, oxygen, and sulfur. In some embodiments, Ring A is a 5,6-fused saturated or partially unsaturated bicyclic heterocyclylenyl or heteroarylenyl containing 1 nitrogen and 1 oxygen heteroatom.

In some embodiments, Ring A is a 5-membered heteroarylenyl having 1-4 heteroatoms independently selected from nitrogen, oxygen, and sulfur. In some embodiments, Ring A is a 5-membered heteroarylenyl having 1-4 heteroatoms nitrogen heteroatoms. In some embodiments, Ring A is a 5-membered heteroarylenyl having 1-2 heteroatoms nitrogen heteroatoms. In some embodiments, Ring A is a 5-membered heteroarylenyl having 1 nitrogen heteroatom and 1 oxygen heteroatom. In some embodiments, Ring A is a 5-membered heteroarylenyl having 1 nitrogen heteroatom and 1 sulfur heteroatom. In some embodiments, Ring A is pyrrolyl, pyrazolyl, imidazolyl, triazolyl, or tetrazolyl. In some embodiments, Ring A is pyrrolyl. In some embodiments, Ring A is pyrazolyl. In some embodiments, Ring A is imidazolyl. In some embodiments, Ring A is triazolyl. In some embodiments, Ring A is tetrazolyl. In some embodiments, Ring A is furanyl, oxazolyl, isoxazolyl, or oxadiazolyl. In some embodiments, Ring A is furanyl. In some embodiments, Ring A is oxazolyl. In some embodiments, Ring A is isoxazolyl. In some embodiments, Ring A is oxadiazolyl. In some embodiments, Ring A is thiophenyl, thiazolyl, isothiazolyl, or thiadiazolyl. In some embodiments, Ring A is thiophenyl. In some embodiments, Ring A is thiazolyl. In some embodiments, Ring A is isothiazolyl. In some embodiments, Ring A is thiadiazolyl.

In some embodiments, Ring A is a 6-membered heteroarylenyl having 1-4 heteroatoms independently selected from nitrogen, oxygen, and sulfur. In some embodiments, Ring A is a 6-membered heteroarylenyl having 1-4 heteroatoms nitrogen heteroatoms. In some embodiments, Ring A is a 6-membered heteroarylenyl having 1-3 heteroatoms nitrogen heteroatoms. In some embodiments, Ring A is pyridinyl, pyrimidinyl, or triazinyl. In some embodiments, Ring A is pyrimidinyl. In some embodiments, Ring A is triazinyl.

In some embodiments, Ring A is a 4-membered saturated or partially unsaturated heterocyclyl having 1-4 heteroatoms independently selected from nitrogen, oxygen, and sulfur. In some embodiments, Ring A is a 7-membered saturated or partially unsaturated heterocyclyl having 1-4 heteroatoms independently selected from nitrogen, oxygen, and sulfur.

In some embodiments, Ring A is a 5-membered saturated or partially unsaturated heterocyclyl having 1-4 heteroatoms independently selected from nitrogen, oxygen, and sulfur. In some embodiments, Ring A is pyrrolidinyl, pyrrolinyl, pyrazolidinyl, pyrazolinyl, imidazolidinyl, or imidazolinyl. In some embodiments, Ring A is pyrrolidinyl. In some embodiments, Ring A is pyrrolinyl. In some embodiments, Ring A is pyrazolidinyl. In some embodiments, Ring A is pyrazolinyl. In some embodiments, Ring A is imidazolidinyl. In some embodiments, Ring A is imidazolinyl. In some embodiments, Ring A is tetrahydrofuranyl, 1,3-dioxolanyl, tetrahydrothiophenyl, 1,2-oxathiolanyl, or 1,3-oxathiolanyl. In some embodiments, Ring A is tetrahydrofuranyl. In some embodiments, Ring A is 1,3-dioxolanyl. In some embodiments, Ring A is tetrahydrothiophenyl. In some embodiments, Ring A is 1,2-oxathiolanyl. In some embodiments, Ring A is 1,3-oxathiolanyl.

In some embodiments, Ring A is a 6-membered saturated or partially unsaturated heterocyclyl having 1-4 heteroatoms independently selected from nitrogen, oxygen, and sulfur. In some embodiments, Ring A is piperidinyl, piperazinyl, tetrahydropyranyl, 2H-pyranyl, 4H-pyranyl, 1,4-dioxanyl, 1,4-dioxinyl, thianyl, 2H-thiopyranyl, 4H-thiopyranyl, 1,3-dithanyl, 1,4-dithanyl, morpholinyl, or thiomorpholinyl. In some embodiments, Ring A is piperidinyl. In some embodiments, Ring A is piperazinyl. In some embodiments, Ring A is tetrahydropyranyl. In some embodiments, Ring A is 2H-pyranyl. In some embodiments, Ring A is 4H-pyranyl. In some embodiments, Ring A is 1,4-dioxanyl. In some embodiments, Ring A is 1,4-dioxinyl. In some embodiments, Ring A is thianyl. In some embodiments, Ring A is 211-thiopyranyl. In some embodiments, Ring A is 411-thiopyranyl. In some embodiments, Ring A is 1,3-dithanyl. In some embodiments, Ring A is 1,4-dithanyl. In some embodiments, Ring A is morpholinyl. In some embodiments, Ring A is thiomorpholinyl. In some embodiments, Ring A is pyrimidonyl or pyridazinonyl. In some embodiments, Ring A is pyrimidonyl. In some embodiments, Ring A is pyridazinonyl.

In some embodiments, Ring A is not phthalimide.

In some embodiments, Ring A is:

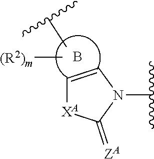

or a pharmaceutically acceptable salt thereof, wherein each R², Ring B, X^{A}, and m is as defined above and described herein both individually and in combination; and:
X^{A} is CR₂, NR, O, or S; and
Z^{A} is O, S, or NR.

As defined above and described herein, Z^{A} is O, S, or NR. In some embodiments, Z^{A} is O. In some embodiments, Z^{A} is S. In some embodiments, Z^{A} is NR.

As defined above and described herein, X^{A} is CR₂, NR, O, or S. In some embodiments, X^{A} is CR₂. In some embodiments, X^{A} is NR. In some embodiments, X^{A} is 0. In some embodiments, X^{A} is S.

In some embodiments, Ring A is:

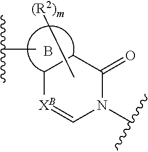

or a pharmaceutically acceptable salt thereof, wherein each R², Ring B, and m is as defined above and described herein both individually and in combination; and:
X^{B} is CR² or N.

As defined above and described herein, X^{B} is CR² or N. In some embodiments, X^{B} is CR². In some embodiments, X^{B} is N.

In some embodiments, Ring A is

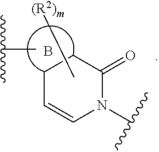

In some embodiments, Ring A is

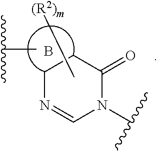

As defined herein and described above, Ring A is a ring selected from phenylenyl, naphthylenyl, pyridinylenyl

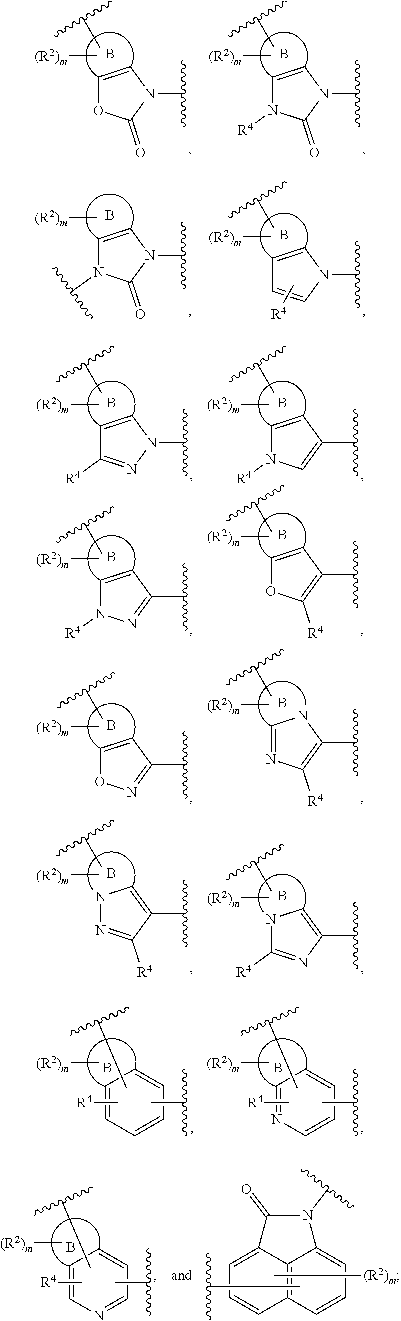

In some embodiments, Ring A is phenylenyl. In some embodiments, Ring A is pyridinylenyl. In some embodiments, Ring A is

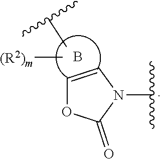

In some embodiments, Ring A is

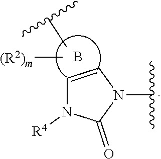

In some embodiments, Ring A is

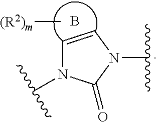

In some embodiments, Ring A is

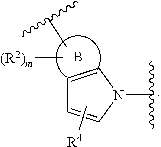

In some embodiments, Ring A is

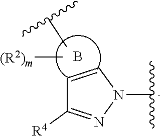

In some embodiments, Ring A is

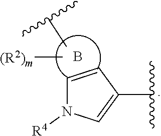

In some embodiments, Ring A is

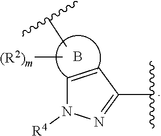

In some embodiments, Ring A is

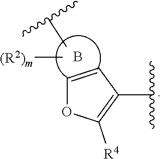

In some embodiments, Ring A is

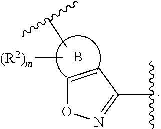

In some embodiments, Ring A is

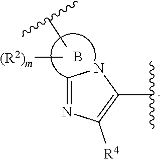

In some embodiments, Ring A is

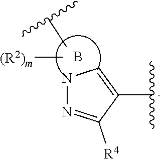

In some embodiments, Ring A is

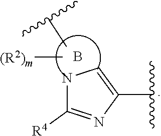

In some embodiments, Ring A is

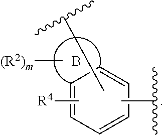

In some embodiments, Ring A is

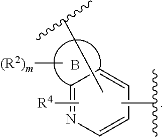

In some embodiments, Ring A is

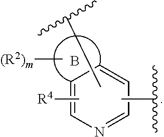

In some embodiments, Ring A is

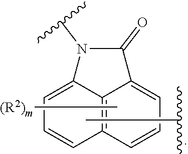

In some embodiments, Ring A is

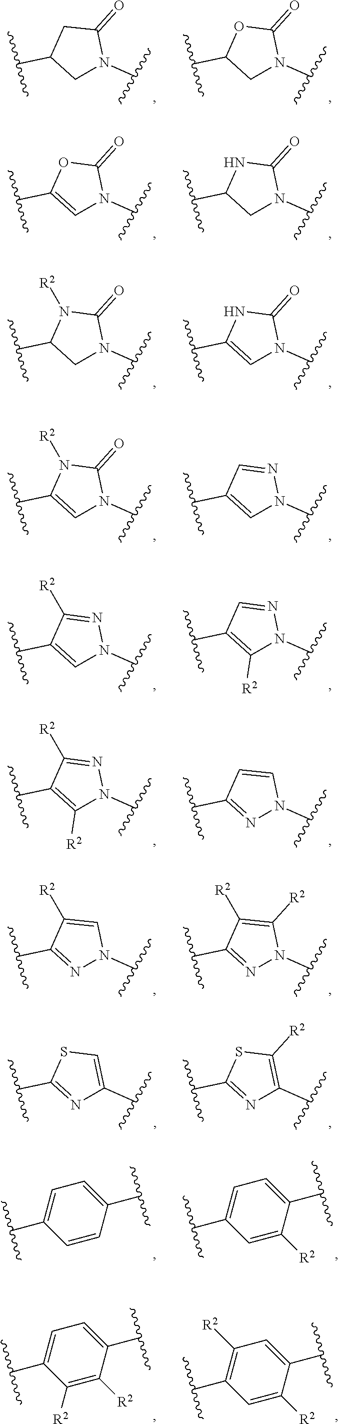

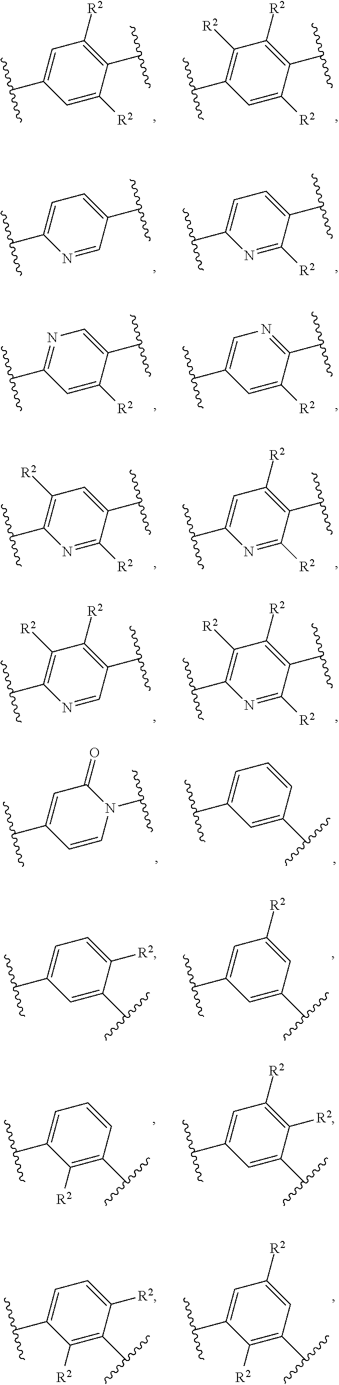

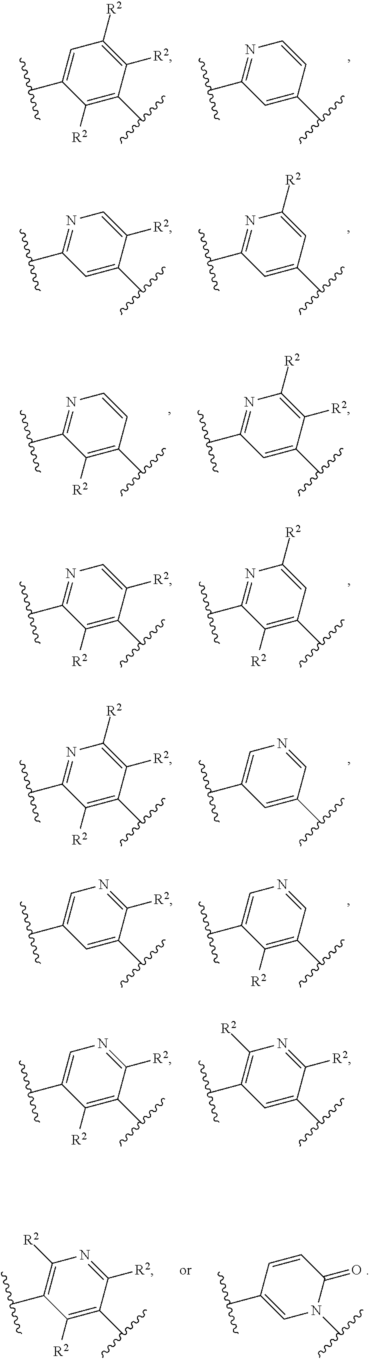

In some embodiments, Ring A is

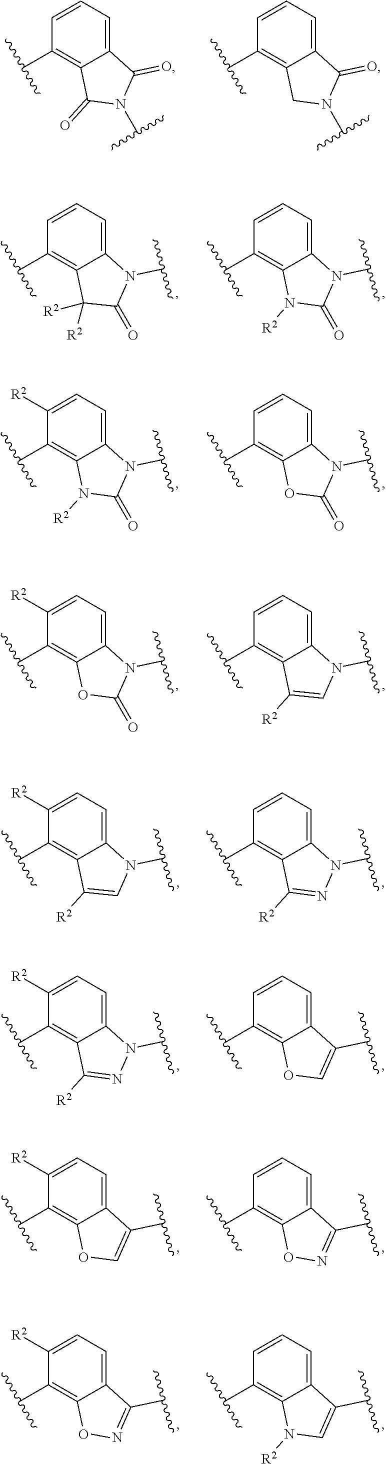

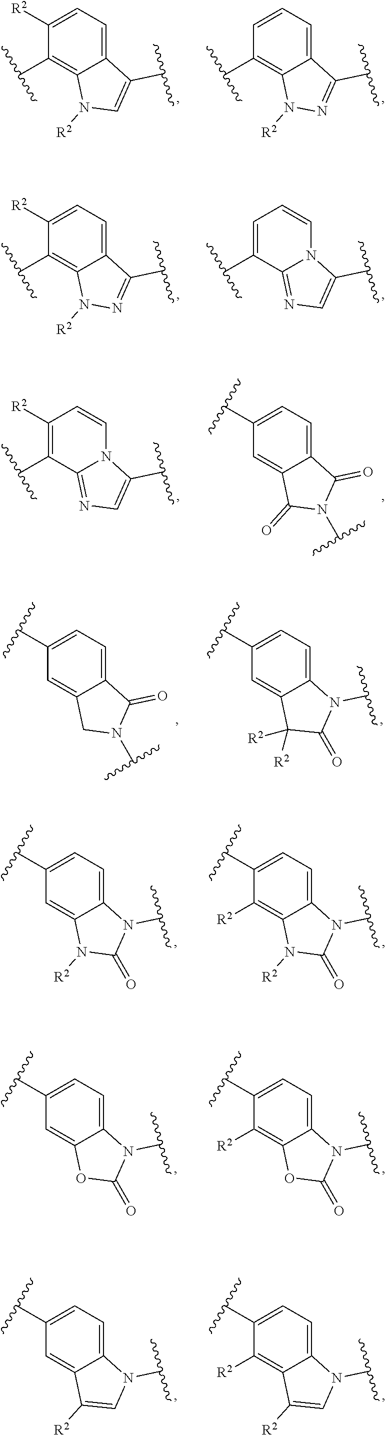

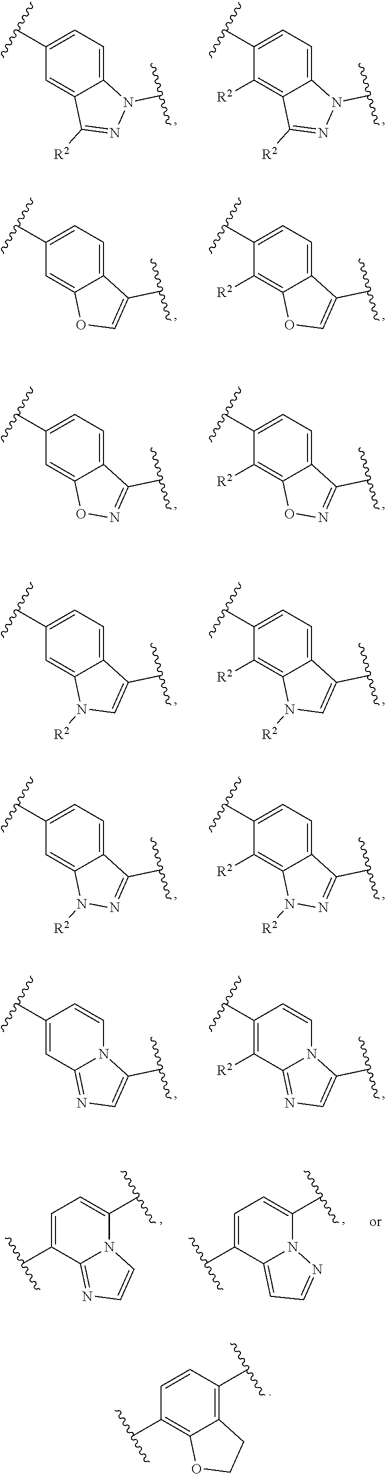

In some embodiments, Ring A is

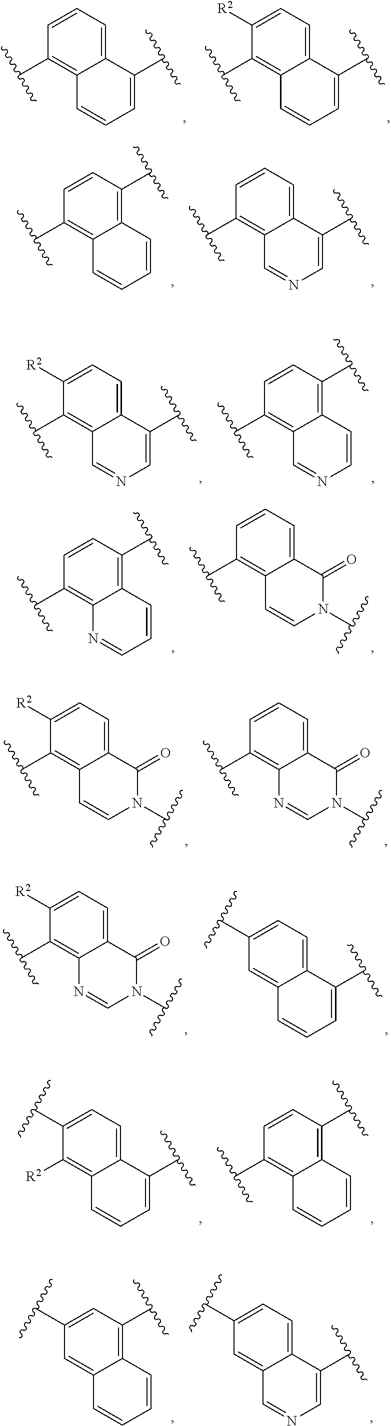

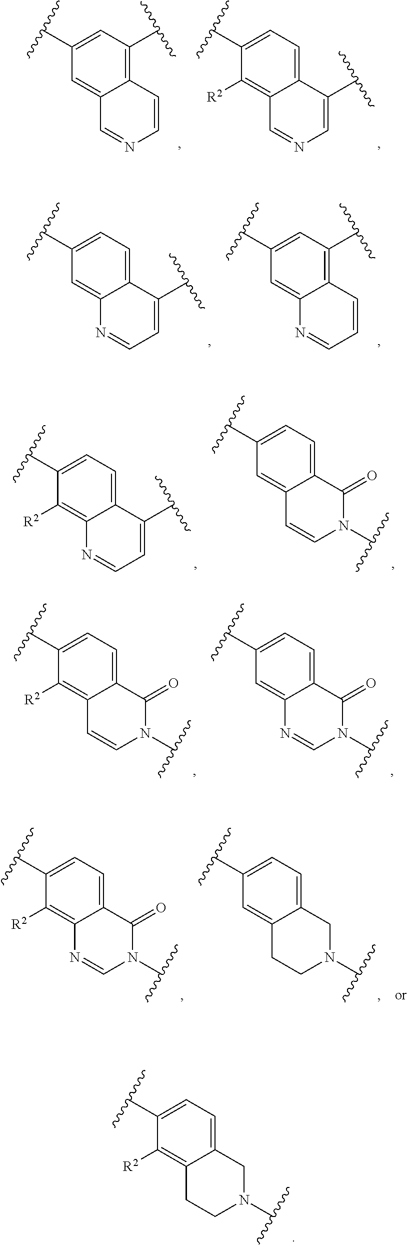

In some embodiments, Ring A is an 10-15 membered saturated or partially unsaturated tricyclic heterocyclylenyl having 1-5 heteroatoms independently selected from nitrogen, oxygen, and sulfur.

In some embodiments, Ring A is a 12-membered saturated or partially unsaturated tricyclic heterocyclylenyl having 1-5 heteroatoms independently selected from nitrogen, oxygen, and sulfur. In some embodiments, Ring A is a 12-membered saturated or partially unsaturated tricyclic heterocyclylenyl having 1-5 nitrogen heteroatoms. In some embodiments, Ring A is a 12-membered saturated or partially unsaturated tricyclic heterocyclylenyl having 3 nitrogen heteroatoms.

In some embodiments, Ring A is a 5,6,5-fused saturated or partially unsaturated tricyclic heterocyclylenyl having 1-5 heteroatoms independently selected from nitrogen, oxygen, and sulfur. In some embodiments, Ring A is a 5,6,5-fused membered saturated or partially unsaturated tricyclic heterocyclylenyl having 1-5 nitrogen heteroatoms. In some embodiments, Ring A is a 5,6,5-fused membered saturated or partially unsaturated tricyclic heterocyclylenyl having 3 nitrogen heteroatoms.

In some embodiments, Ring A is a 5,5,6-fused saturated or partially unsaturated tricyclic heterocyclylenyl having 1-5 heteroatoms independently selected from nitrogen, oxygen, and sulfur. In some embodiments, Ring A is a 5,5,6-fused membered saturated or partially unsaturated tricyclic heterocyclylenyl having 1-5 nitrogen heteroatoms. In some embodiments, Ring A is a 5,5,6-fused membered saturated or partially unsaturated tricyclic heterocyclylenyl having 3 nitrogen heteroatoms.

In some embodiments, Ring A is a 13-membered saturated or partially unsaturated tricyclic heterocyclylenyl having 1-5 heteroatoms independently selected from nitrogen, oxygen, and sulfur. In some embodiments, Ring A is a 13-membered saturated or partially unsaturated tricyclic heterocyclylenyl having 1-5 nitrogen heteroatoms. In some embodiments, Ring A is a 13-membered saturated or partially unsaturated tricyclic heterocyclylenyl having 3 nitrogen heteroatoms.

In some embodiments, Ring A is a 5,6,6-fused saturated or partially unsaturated tricyclic heterocyclylenyl having 1-5 heteroatoms independently selected from nitrogen, oxygen, and sulfur. In some embodiments, Ring A is a 5,6,6-fused membered saturated or partially unsaturated tricyclic heterocyclylenyl having 1-5 nitrogen heteroatoms. In some embodiments, Ring A is a 5,6,6-fused membered saturated or partially unsaturated tricyclic heterocyclylenyl having 3 nitrogen heteroatoms.

In some embodiments, Ring A is a 6,5,6-fused saturated or partially unsaturated tricyclic heterocyclylenyl having 1-5 heteroatoms independently selected from nitrogen, oxygen, and sulfur. In some embodiments, Ring A is a 6,5,6-fused membered saturated or partially unsaturated tricyclic heterocyclylenyl having 1-5 nitrogen heteroatoms. In some embodiments, Ring A is a 6,5,6-fused membered saturated or partially unsaturated tricyclic heterocyclylenyl having 3 nitrogen heteroatoms.

In some embodiments, Ring A is a 14-membered saturated or partially unsaturated tricyclic heterocyclylenyl having 1-5 heteroatoms independently selected from nitrogen, oxygen, and sulfur.

In some embodiments, Ring A is or an 10-15 membered tricyclic heteroarylenyl having 1-5 heteroatoms independently selected from nitrogen, oxygen, and sulfur.

In some embodiments, Ring A is a 12-membered saturated or partially unsaturated tricyclic heteroarylenyl having 1-5 heteroatoms independently selected from nitrogen, oxygen, and sulfur. In some embodiments, Ring A is a 12-membered saturated or partially unsaturated tricyclic heteroarylenyl having 1-5 nitrogen heteroatoms. In some embodiments, Ring A is a 12-membered saturated or partially unsaturated tricyclic heteroarylenyl having 3 nitrogen heteroatoms.

In some embodiments, Ring A is a 5,6,5-fused saturated or partially unsaturated tricyclic heteroarylenyl having 1-5 heteroatoms independently selected from nitrogen, oxygen, and sulfur. In some embodiments, Ring A is a 5,6,5-fused membered saturated or partially unsaturated tricyclic heteroarylenyl having 1-5 nitrogen heteroatoms. In some embodiments, Ring A is a 5,6,5-fused membered saturated or partially unsaturated tricyclic heteroarylenyl having 3 nitrogen heteroatoms.

In some embodiments, Ring A is a 5,5,6-fused saturated or partially unsaturated tricyclic heteroarylenyl having 1-5 heteroatoms independently selected from nitrogen, oxygen, and sulfur. In some embodiments, Ring A is a 5,5,6-fused membered saturated or partially unsaturated tricyclic heteroarylenyl having 1-5 nitrogen heteroatoms. In some embodiments, Ring A is a 5,5,6-fused membered saturated or partially unsaturated tricyclic heteroarylenyl having 3 nitrogen heteroatoms.

In some embodiments, Ring A is a 13-membered saturated or partially unsaturated tricyclic heteroarylenyl having 1-5 heteroatoms independently selected from nitrogen, oxygen, and sulfur. In some embodiments, Ring A is a 13 membered saturated or partially unsaturated tricyclic heteroarylenyl having 1-5 nitrogen heteroatoms. In some embodiments, Ring A is a 13-membered saturated or partially unsaturated tricyclic heteroarylenyl having 3 nitrogen heteroatoms.

In some embodiments, Ring A is a 5,6,6-fused saturated or partially unsaturated tricyclic heteroarylenyl having 1-5 heteroatoms independently selected from nitrogen, oxygen, and sulfur. In some embodiments, Ring A is a 5,6,6-fused membered saturated or partially unsaturated tricyclic heteroarylenyl having 1-5 nitrogen heteroatoms. In some embodiments, Ring A is a 5,6,6-fused membered saturated or partially unsaturated tricyclic heteroarylenyl having 3 nitrogen heteroatoms.

In some embodiments, Ring A is a 6,5,6-fused saturated or partially unsaturated tricyclic heteroarylenyl having 1-5 heteroatoms independently selected from nitrogen, oxygen, and sulfur. In some embodiments, Ring A is a 6,5,6-fused membered saturated or partially unsaturated tricyclic heteroarylenyl having 1-5 nitrogen heteroatoms. In some embodiments, Ring A is a 6,5,6-fused membered saturated or partially unsaturated tricyclic heteroarylenyl having 3 nitrogen heteroatoms.

In some embodiments, Ring A is a 14-membered saturated or partially unsaturated tricyclic heteroarylenyl having 1-5 heteroatoms independently selected from nitrogen, oxygen, and sulfur.

In some embodiments, Ring A is of formula ABC-1 or ABC-2:

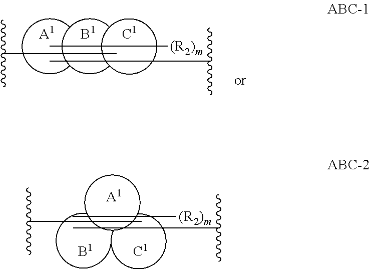

or a pharmaceutically acceptable salt thereof, wherein each R¹ is R² and m is as defined above and described herein both individually and in combination; and:
each of Ring A¹, Ring B¹, and Ring C¹ is independently a fused ring selected from a 4-7 membered saturated or partially unsaturated carbocyclylenyl; benzo; a 4-7 membered saturated or partially unsaturated heterocyclylenyl having 1-5 heteroatoms independently selected from nitrogen, oxygen, and sulfur; or a 5-6 membered heteroarylenyl having 1-5 heteroatoms independently selected from nitrogen, oxygen, and sulfur.

In some embodiments, Ring A¹ is a fused 4-7 membered saturated or partially unsaturated carbocyclylenyl. In some embodiments, Ring A¹ is benzo. In some embodiments, Ring A¹ is a fused 4- to 7-membered saturated or partially unsaturated heterocyclylenyl having 1-5 heteroatoms independently selected from nitrogen, oxygen, and sulfur. In some embodiments, Ring A¹ is a fused 5-6 membered heteroarylenyl having 1-5 heteroatoms independently selected from nitrogen, oxygen, and sulfur.

In some embodiments, Ring B¹ is a fused 4-7 membered saturated or partially unsaturated carbocyclylenyl. In some embodiments, Ring B1 is benzo. In some embodiments, Ring B¹ is a fused 4- to 7-membered saturated or partially unsaturated heterocyclylenyl having 1-5 heteroatoms independently selected from nitrogen, oxygen, and sulfur. In some embodiments, Ring B¹ is a fused 5-6 membered heteroarylenyl having 1-5 heteroatoms independently selected from nitrogen, oxygen, and sulfur.

In some embodiments, Ring C¹ is a fused 4-7 membered saturated or partially unsaturated carbocyclylenyl. In some embodiments, Ring C¹ is benzo. In some embodiments, Ring C¹ is a fused 4- to 7-membered saturated or partially unsaturated heterocyclylenyl having 1-5 heteroatoms independently selected from nitrogen, oxygen, and sulfur. In some embodiments, Ring C¹ is a fused 5-6 membered heteroarylenyl having 1-5 heteroatoms independently selected from nitrogen, oxygen, and sulfur.

In some embodiments, Ring A is

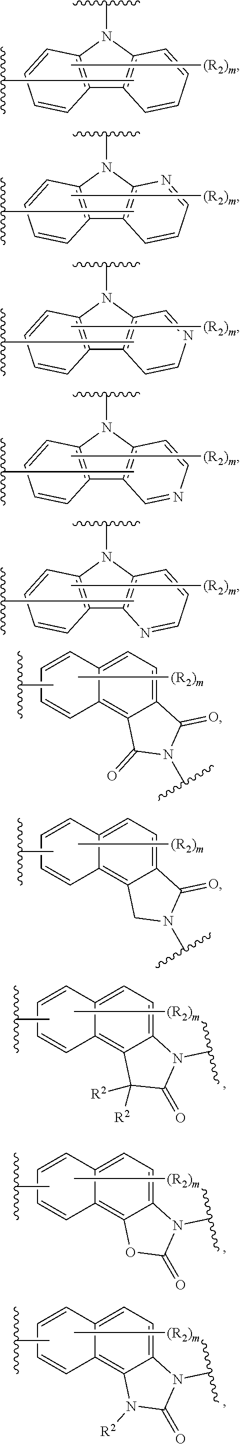

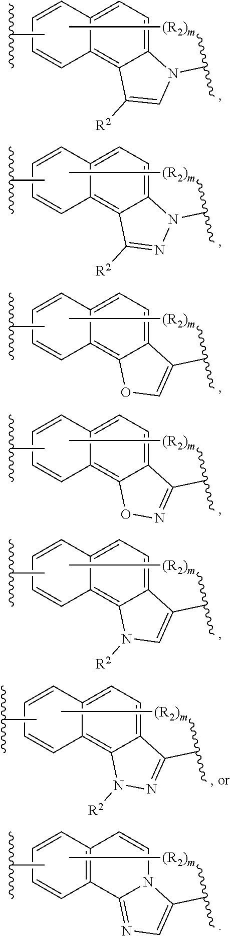

In some embodiments, Ring A is a bicyclic 9-10 membered partially unsaturated heterocyclyl or heteroaryl having 1-4 heteroatoms independently selected from nitrogen, oxygen, and sulfur. In some embodiments, Ring A is a tricyclic 12-membered partially unsaturated heterocyclyl or heteroaryl having 1-4 heteroatoms independently selected from nitrogen, oxygen, and sulfur.

In some embodiments, Ring A is as depicted in the compounds of Table 1, below.

As defined herein and described above, Ring B is a fused ring selected from benzo, a 5-6 membered saturated or partially unsaturated heterocyclyl having 1-3 heteroatoms independently selected from nitrogen, oxygen, and sulfur, or a 5-6 membered heteroaryl containing 1-4 heteroatoms independently selected from nitrogen, oxygen, and sulfur.

In some embodiments, Ring B is benzo. In some embodiments, Ring B is a 5-6 membered saturated or partially unsaturated heterocyclyl having 1-3 heteroatoms independently selected from nitrogen, oxygen, and sulfur. In some embodiments, Ring B is a 5-6 membered heteroaryl containing 1-4 heteroatoms independently selected from nitrogen, oxygen, and sulfur.

In some embodiments, Ring B is as depicted in the compounds of Table 1, below.

In some embodiments,

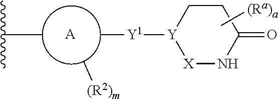

is

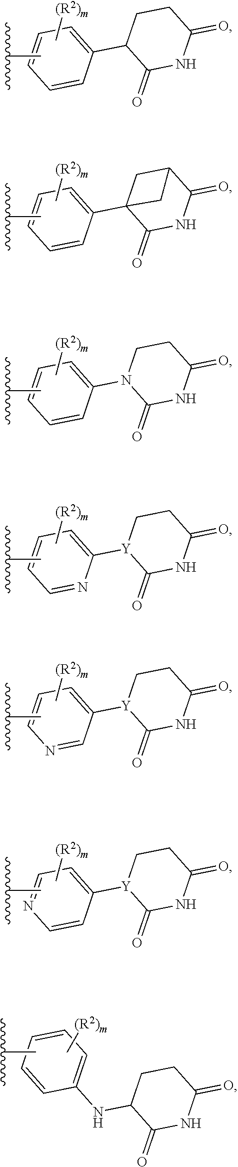

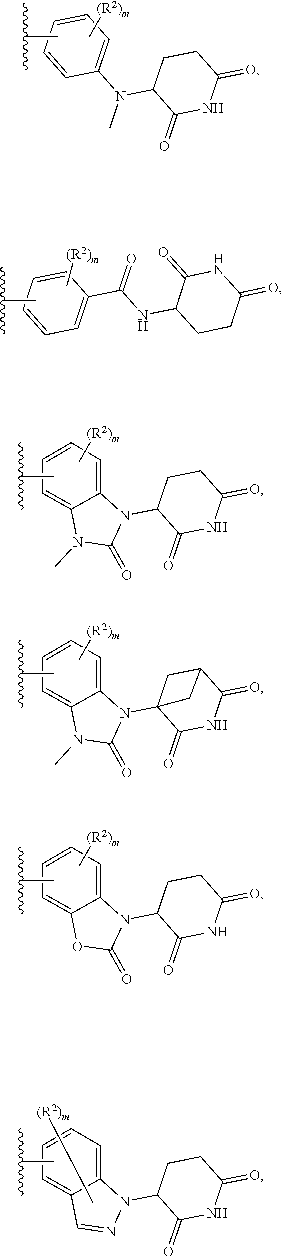

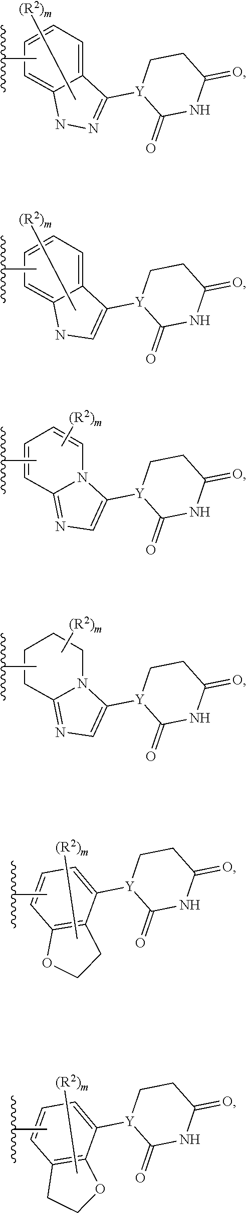

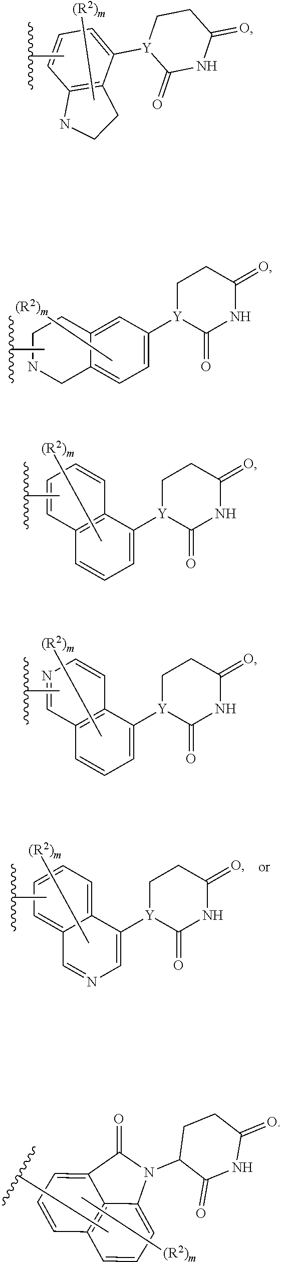

In some embodiments,

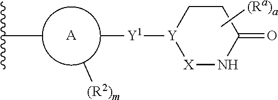

is

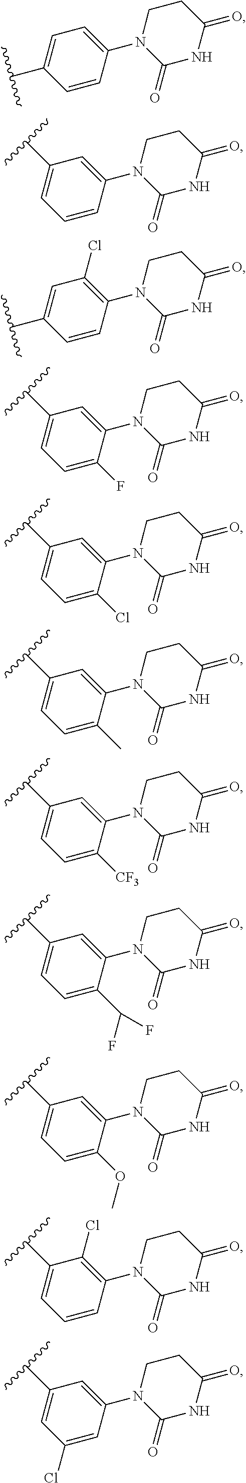

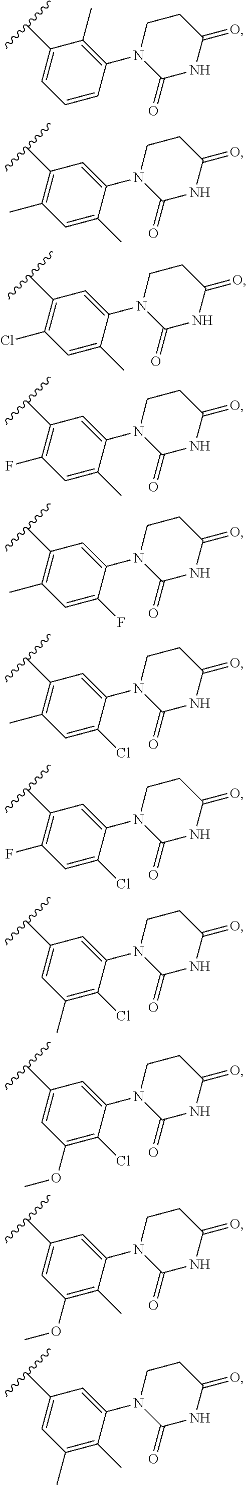

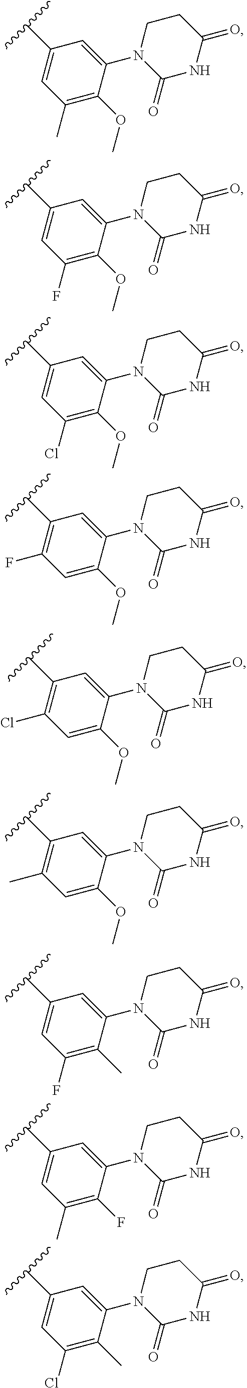

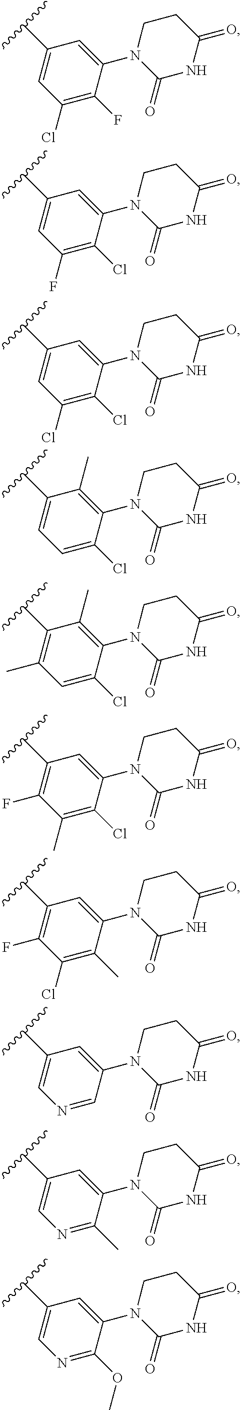

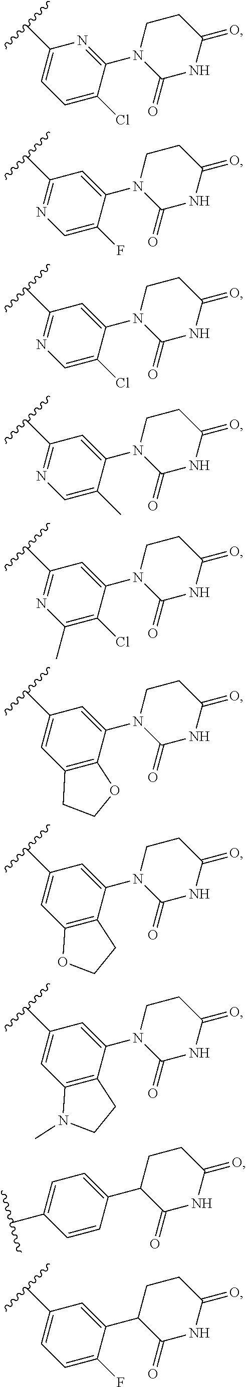

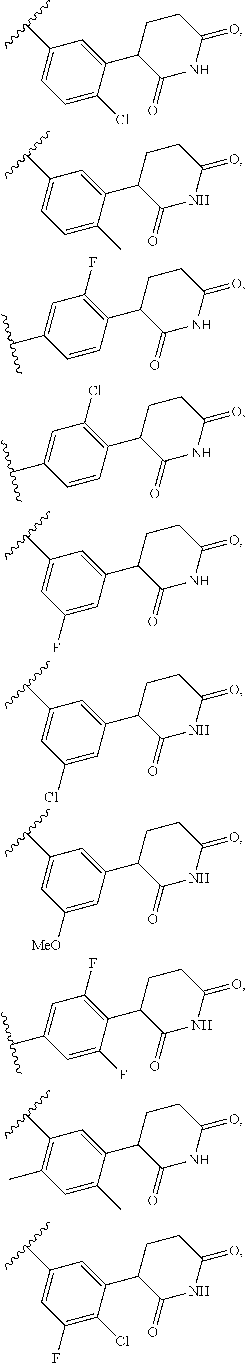

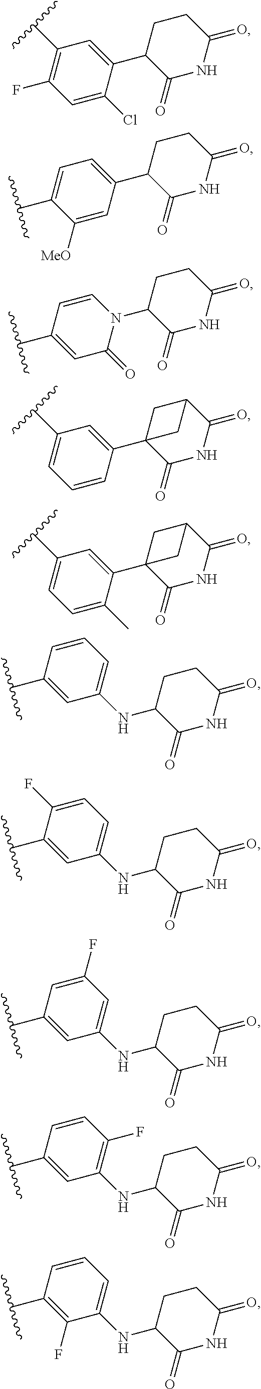

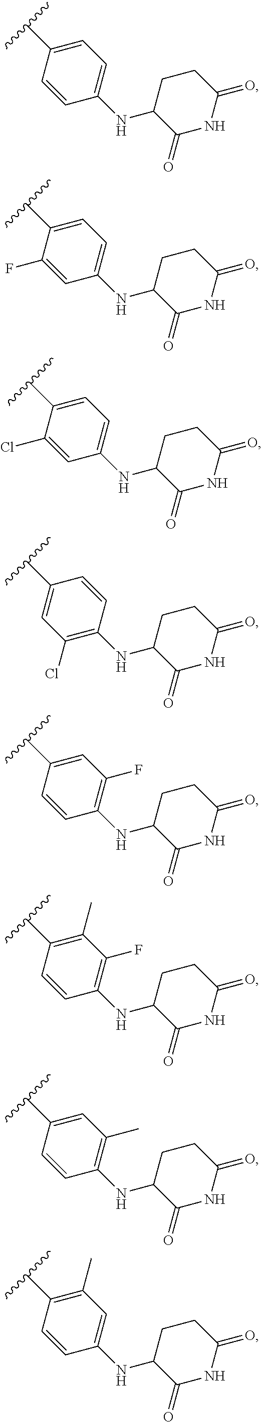

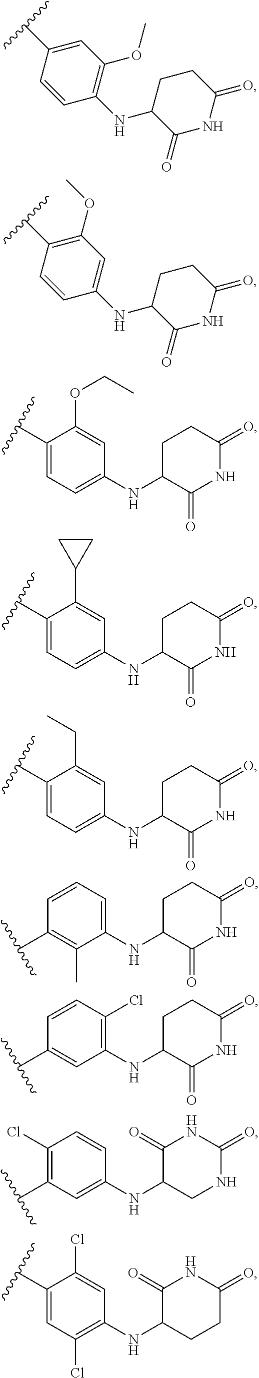

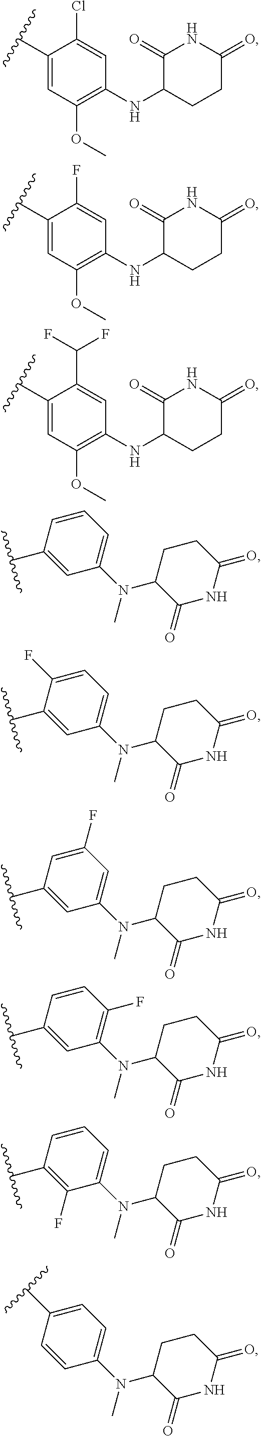

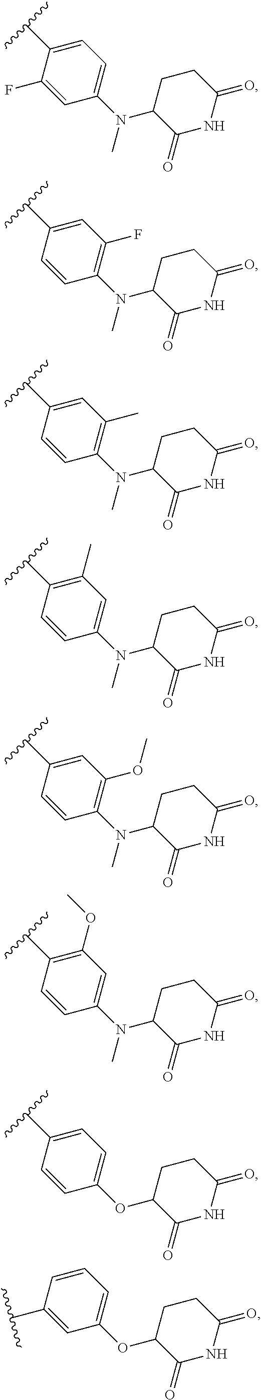

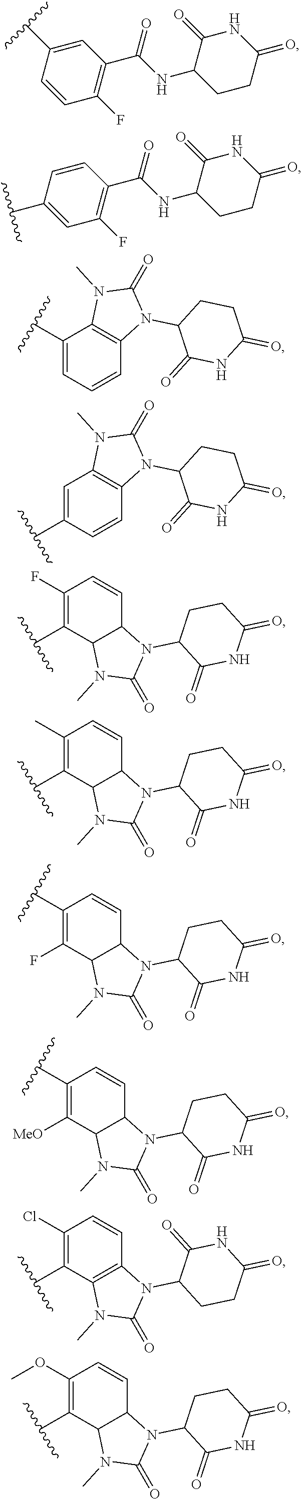

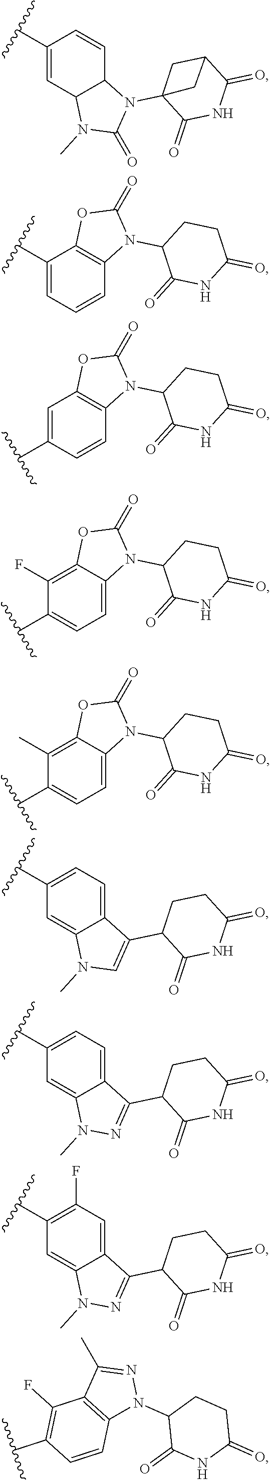

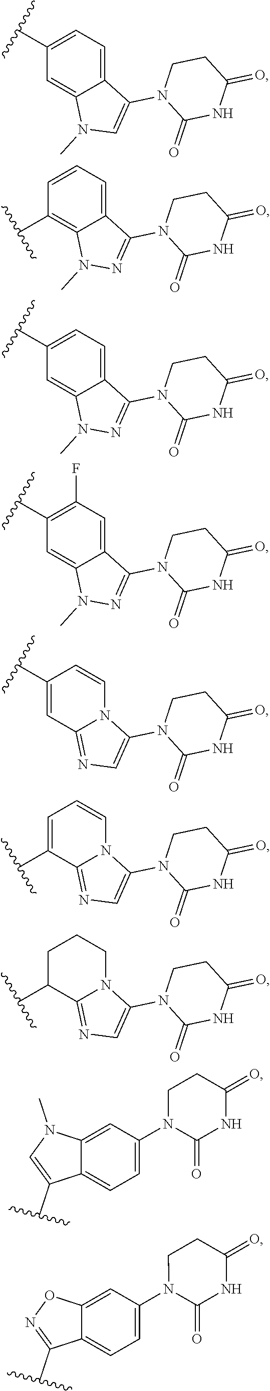

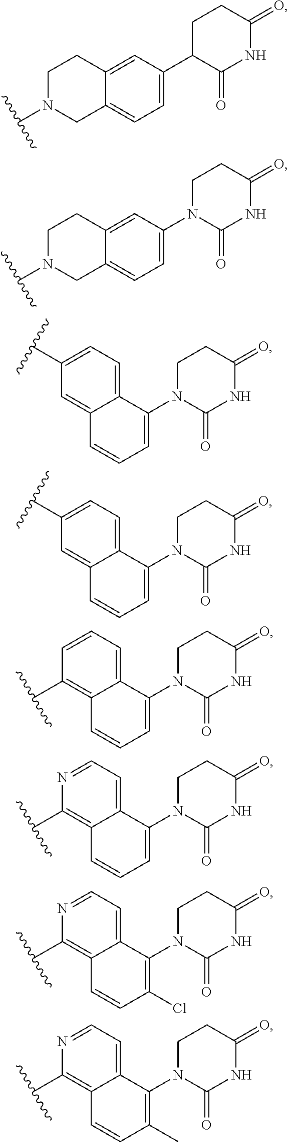

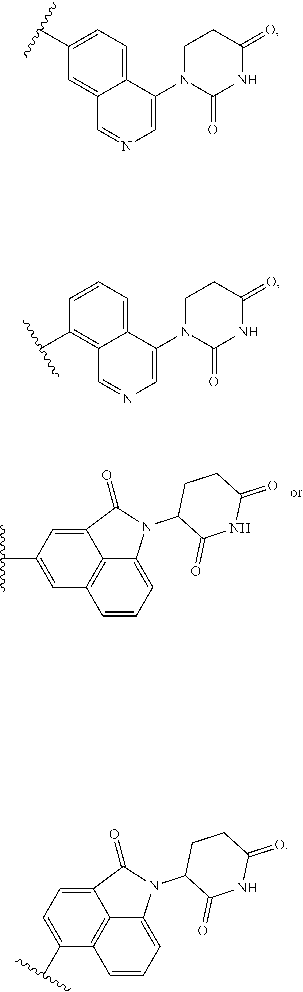 [SMILES: *c1ccc2c(N3CCC(=O)NC3=O)cncc2c1 ; *c1cccc2c(N3CCC(=O)NC3=O)cncc12 ; *c1cc2c3c(cccc3c1)N(C1CCC(=O)NC1=O)C2=O ; *c1ccc2c3c(cccc13)N(C1CCC(=O)NC1=O)C2=O]

As defined herein and described above, Ring C is phenylenyl, pyridylenyl, an 8-15 membered saturated or partially unsaturated monocyclic, bicyclic, bridged bicyclic, spirocyclic, or tricyclic carbocyclylenyl or heterocyclylenyl having 1-3 heteroatoms independently selected from nitrogen, oxygen, and sulfur, or a 5-10 membered monocyclic or bicyclic heteroarylenyl having 1-3 heteroatoms independently selected from nitrogen, oxygen, and sulfur.

In some embodiments, Ring C is an 8-15 membered saturated or partially unsaturated monocyclic, bicyclic, bridged bicyclic, spirocyclic, or tricyclic carbocyclylenyl. In some embodiments, Ring C is an 8-15 membered saturated or partially unsaturated monocyclic, bicyclic, bridged bicyclic, spirocyclic, or tricyclic heterocyclylenyl having 1-3 heteroatoms independently selected from nitrogen, oxygen, and sulfur. In some embodiments, Ring C is an 8-membered saturated or partially unsaturated bicyclic heterocyclylenyl having 1-3 heteroatoms independently selected from nitrogen, oxygen, and sulfur. In some embodiments, Ring C is a 5-10 membered monocyclic or bicyclic heteroarylenyl having 1-3 heteroatoms independently selected from nitrogen, oxygen, and sulfur. In some embodiments, Ring C is an 8-membered bicyclic heteroarylenyl having 1-3 heteroatoms independently selected from nitrogen, oxygen, and sulfur. In some embodiments, Ring C is a 5-membered heteroarylenyl having 1-3 heteroatoms independently selected from nitrogen, oxygen, and sulfur.

As defined herein and described above, Ring C is phenylenyl, pyridylenyl, a 9-10 membered saturated or partially unsaturated bicyclic heterocyclylenyl having 1-3 heteroatoms independently selected from nitrogen, oxygen, and sulfur, 8-10 membered bicyclic heteroarylenyl having 1-3 heteroatoms independently selected from nitrogen, oxygen, and sulfur, 12-15 membered saturated or partially unsaturated tricyclic spirocyclic heterocyclylenyl having 1-3 heteroatoms independently selected from nitrogen, oxygen.

In some embodiments, Ring C is phenylenyl, a 9-10 membered saturated or partially unsaturated bicyclic heterocyclylenyl having 1-3 heteroatoms independently selected from nitrogen, oxygen, and sulfur, 8-10 membered bicyclic heteroarylenyl having 1-3 heteroatoms independently selected from nitrogen, oxygen, and sulfur, 12-15 membered saturated or partially unsaturated tricyclic spirocyclic heterocyclylenyl having 1-3 heteroatoms independently selected from nitrogen, oxygen.

In some embodiments, Ring C is phenylenyl. In some embodiments, Ring C is pyridylenyl. In some embodiments, Ring C is a 9-10 membered saturated or partially unsaturated bicyclic heterocyclylenyl having 1-3 heteroatoms independently selected from nitrogen, oxygen, and sulfur. In some embodiments, Ring C is a 8-10 membered bicyclic heteroarylenyl having 1-3 heteroatoms independently selected from nitrogen, oxygen, and sulfur. In some embodiments, Ring C is 12-15 membered saturated or partially unsaturated tricyclic spirocyclic heterocyclylenyl having 1-3 heteroatoms independently selected from nitrogen, oxygen.

In some embodiments, Ring C is a 9-membered saturated or partially unsaturated bicyclic heterocyclylenyl having 1-3 heteroatoms independently selected from nitrogen, oxygen, and sulfur. In some embodiments, Ring C is a 9-membered partially unsaturated bicyclic heterocyclylenyl having 1-3 heteroatoms independently selected from nitrogen, oxygen, and sulfur. In some embodiments, Ring C is a 9-membered partially unsaturated bicyclic heterocyclylenyl having 1-2 nitrogen heteroatoms. In some embodiments, Ring C is a 9-membered partially unsaturated bicyclic heterocyclylenyl having 1 nitrogen heteroatom.

In some embodiments, Ring C is 14-membered partially unsaturated tricyclic spirocyclic heterocyclylenyl having 1-3 heteroatoms independently selected from nitrogen, oxygen. In some embodiments, Ring C is 14-membered partially unsaturated tricyclic spirocyclic heterocyclylenyl having 1-2 nitrogen heteroatoms.

In some embodiments, Ring C is a N-linked ring. In some embodiments, Ring C is a C-linked ring. In some embodiments, Ring C is a N-linked 9-membered partially unsaturated bicyclic heterocyclylenyl having 1-3 heteroatoms independently selected from nitrogen, oxygen, and sulfur. In some embodiments, Ring C is a C-linked 9-membered partially unsaturated bicyclic heterocyclylenyl having 1-3 heteroatoms independently selected from nitrogen, oxygen, and sulfur.

In some embodiments, Ring C is

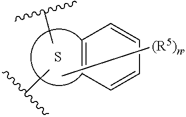

wherein both points of attachment to Ring C are on Ring S, and wherein Ring S is 5-6 membered heterocyclylenyl, 5-6 membered heteroarylenyl, or 10-12 membered spirocyclic heterocyclylenyl; each R⁵ is halogen; and n is 0 or 1.

In some embodiments, each 5-6 membered heterocyclylenyl, 5-6 membered heteroarylenyl, or 10-12 membered spirocyclic heterocyclylenyl contains one or two ring nitrogens. In some embodiments, each point of attachment to Ring C is on a ring nitrogen on Ring S. In some embodiments, each point of attachment to Ring C is on a ring carbon on Ring S. In some embodiments, one point of attachment to Ring C is on a ring nitrogen on Ring S, and the other is on a ring carbon on Ring S.

In some embodiments, Ring S is 10-12 membered spirocyclic heterocyclylenyl containing one or two ring nitrogens.

In some embodiments, Ring C is

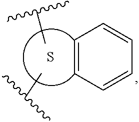

wherein both points of attachment to Ring C are on Ring S, and wherein Ring S is 5-6 membered heterocyclylenyl, 5 or 6-membered heteroarylenyl, or 10-12 membered spirocyclic heterocyclylenyl. In some embodiments, each 5-6 membered heterocyclylenyl, 5-6 membered heteroarylenyl, or 10-12 membered spirocyclic heterocyclylenyl contains one or two ring nitrogens. In some embodiments, each point of attachment to Ring C is on a ring nitrogen on Ring S. In some embodiments, each point of attachment to Ring C is on a ring carbon on Ring S. In some embodiments, one point of attachment to Ring C is on a ring nitrogen on Ring S, and the other is on a ring carbon on Ring S.

In some embodiments, S is 10-12 membered spirocyclic heterocyclylenyl containing one or two ring nitrogens.

In some embodiments, Ring C is

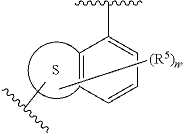

wherein Ring S is 5-6 membered heterocyclylenyl, 5-6 membered heteroarylenyl, or 10-12 membered spirocyclic heterocyclylenyl; each R is halogen; and n is 0 or 1. In some embodiments, each 5 or 6-membered heterocyclylenyl, 5-6 membered heteroarylenyl, or 10-12 membered spirocyclic heterocyclylenyl contains one or two ring nitrogens. In some embodiments, the point of attachment to Ring C on Ring S is on a ring nitrogen. In some embodiments, the point of attachment to Ring C on Ring S is on a ring carbon.

In some embodiments, Ring S is 5-6 membered heterocyclylenyl containing one or two ring heteroatoms selected from nitrogen and oxygen.

In some embodiments, Ring C is

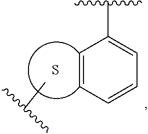

wherein Ring S is 5-6 membered heterocyclylenyl, 5-6 membered heteroarylenyl, or 10-12 membered spirocyclic heterocyclylenyl. In some embodiments, each 5-6 membered heterocyclylenyl, 5-6 membered heteroarylenyl, or 10-12 membered spirocyclic heterocyclylenyl contains one or two ring nitrogens. In some embodiments, the point of attachment to Ring C on Ring S is on a ring nitrogen. In some embodiments, the point of attachment to Ring C on Ring S is on a ring carbon.

In some embodiments, Ring S is 5-6 membered heterocyclylenyl containing one or two ring heteroatoms selected from nitrogen and oxygen.

In some embodiments, Ring C is

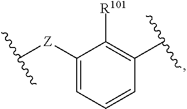

wherein Z is —C(R¹⁰³)₂—, —O— or NR¹⁰²; R¹⁰¹ is H, halogen, C₁₋₃ alkoxy, or C₁₋₃ alkyl optionally substituted with one or more halogen; R¹⁰² is H or C₁₋₃ alkyl, and each R¹⁰³ is independently H, halogen, C₁₋₃ alkoxy, or C₁₋₃ alkyl optionally substituted with one or more halogen.

In some embodiments, Ring C is

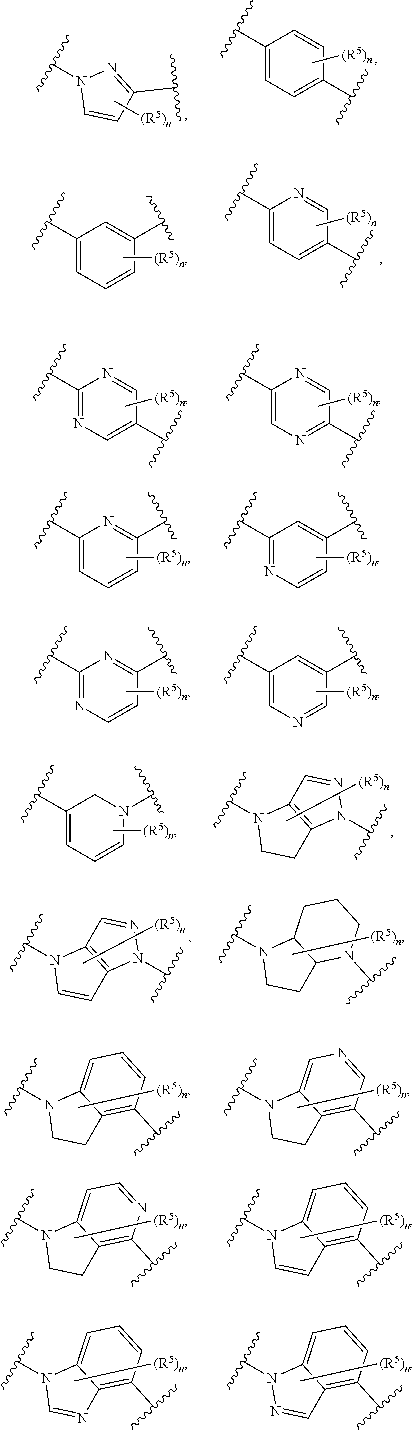

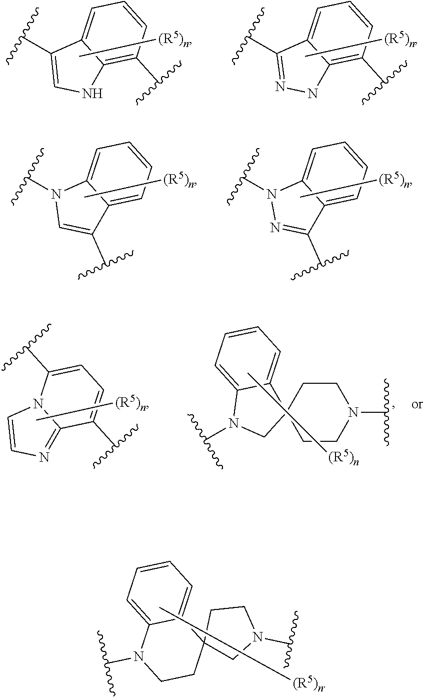

In some embodiments, Ring C is

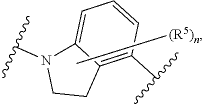

wherein each R⁵ is halogen and n is 0 or 1.

In some embodiments, Ring C is

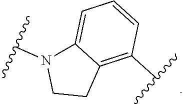 [SMILES: *c1cccc2c1CCN2*]

In some embodiments, Ring C is as depicted in the compounds of Table 1, below.

In some embodiments, Ring C and it's R⁵ substituents are

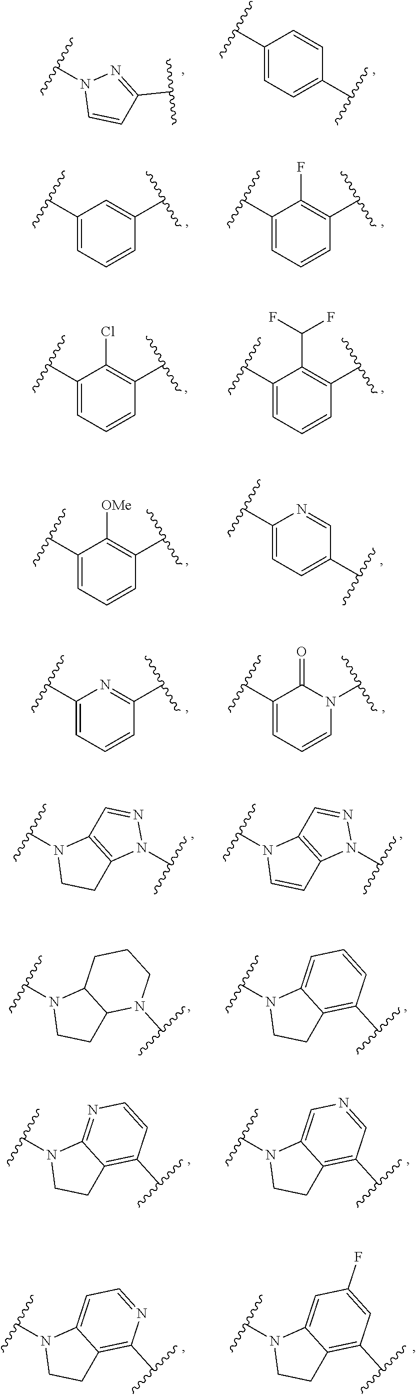

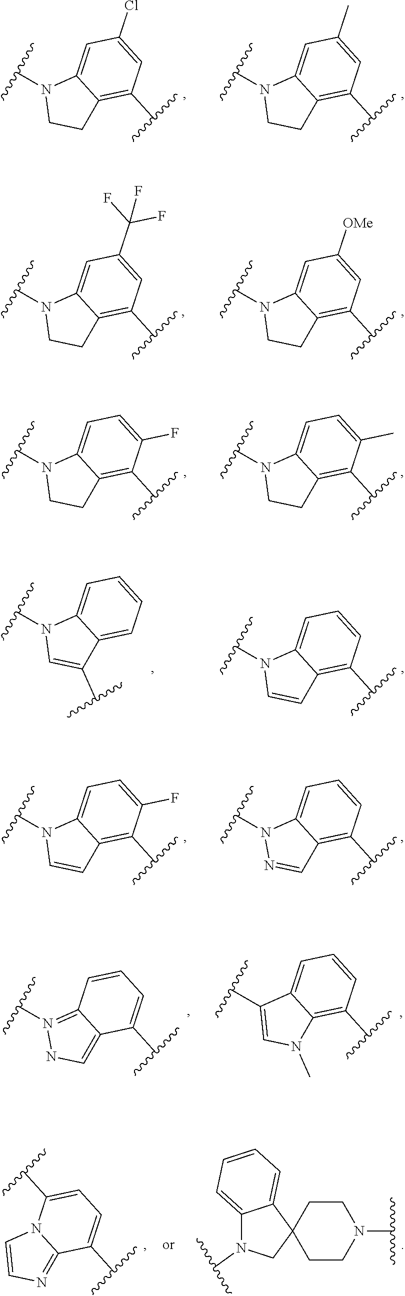

In some embodiments, X² and Ring C are

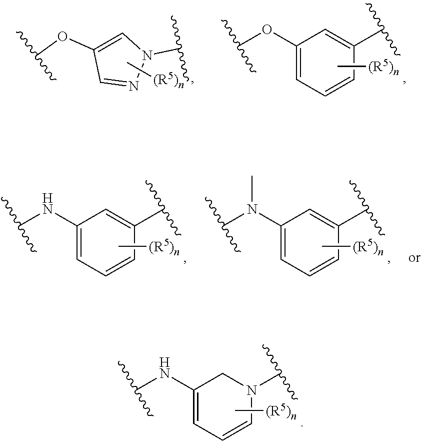

In some embodiments, X² and Ring C, and it's R⁵ substituents are

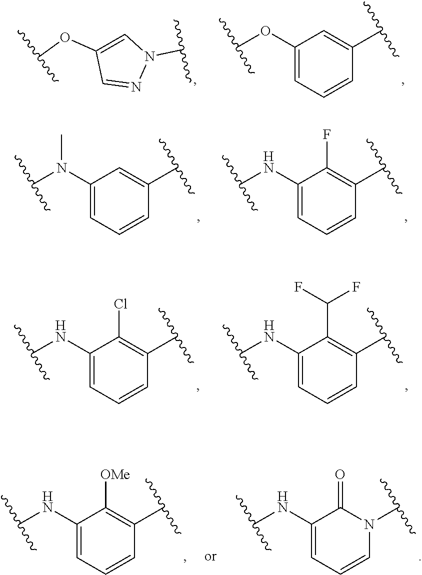

As defined herein and described above, Ring D is a bivalent ring selected from phenylenyl, 4-7 membered saturated or partially unsaturated carbocyclylenyl or heterocyclylenyl having 1-2 heteroatoms independently selected from nitrogen, oxygen, and sulfur, and a 5-6 membered heteroarylenyl ring having 1-4 heteroatoms independently selected from nitrogen, oxygen, and sulfur.

In some embodiments, Ring D is phenylenyl. In some embodiments, Ring D is a 4-7 membered saturated or partially unsaturated carbocyclylenyl. In some embodiments, Ring D is a 4-7 membered saturated or partially unsaturated heterocyclylenyl having 1-2 heteroatoms independently selected from nitrogen, oxygen, and sulfur. In some embodiments, Ring D is a 5-6 membered heteroarylenyl ring having 1-4 heteroatoms independently selected from nitrogen, oxygen, and sulfur.

In some embodiments, Ring D is as depicted in the compounds of Table 1, below.

As defined herein and described above, Ring E is a bivalent ring selected from phenylenyl, naphthylenyl, 4-7 membered saturated or partially unsaturated carbocyclylenyl or heterocyclylenyl having 1-2 heteroatoms independently selected from nitrogen, oxygen, and sulfur, 5-6 membered heteroarylenyl ring having 1-4 heteroatoms independently selected from nitrogen, oxygen, and sulfur, a 9-10 membered saturated or partially unsaturated bicyclic heterocyclylenyl having 1-3 heteroatoms independently selected from nitrogen, oxygen, and sulfur, and 8-10 membered bicyclic heteroarylenyl having 1-3 heteroatoms independently selected from nitrogen, oxygen, and sulfur.

In some embodiments, Ring E is phenylenyl. In some embodiments, Ring E is naphthylenyl. In some embodiments, Ring E is a 4-7 membered saturated or partially unsaturated carbocyclylenyl. In some embodiments, Ring E is a 4-7 membered saturated or partially unsaturated heterocyclylenyl having 1-2 heteroatoms independently selected from nitrogen, oxygen, and sulfur. In some embodiments, Ring E is a 5-6 membered heteroarylenyl ring having 1-4 heteroatoms independently selected from nitrogen, oxygen, and sulfur. In some embodiments, Ring E is a 9-10 membered saturated or partially unsaturated bicyclic heterocyclylenyl having 1-4 heteroatoms independently selected from nitrogen, oxygen, and sulfur. In some embodiments, Ring E is an 8-10 membered bicyclic heteroarylenyl having 1-4 heteroatoms independently selected from nitrogen, oxygen, and sulfur.

In some embodiments, Ring E is a 10-membered bicyclic heteroarylenyl having 1-4 nitrogen atoms.

In some embodiments, Ring E is as depicted in the compounds of Table 1, below.

As defined herein and described above, Ring F is phenyl, 4-7 membered saturated or partially unsaturated carbocyclyl or heterocyclyl having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, Ring F is phenyl. In some embodiments, Ring F is a 4-7 membered saturated or partially unsaturated carbocyclyl. In some embodiments, Ring F is a 4-7 membered saturated or partially unsaturated heterocyclyl having 1-2 heteroatoms independently selected from nitrogen, oxygen, and sulfur. In some embodiments, Ring F is a 5-6 membered heteroaryl ring having 1-4 heteroatoms independently selected from nitrogen, oxygen, and sulfur.

In some embodiments, Ring F is a 6-membered heteroaryl ring having 1-2 nitrogen heteroatoms.

In some embodiments, Ring F is

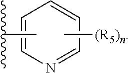

In some embodiments, Ring F is

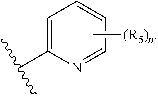

In some embodiments, Ring F is

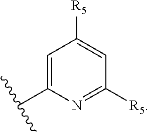

In some embodiments, Ring F and it's R⁵ substituents are

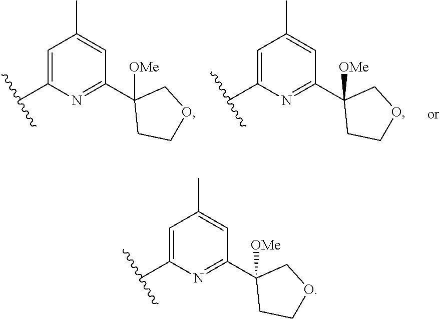

In some embodiments, Ring F is as depicted in the compounds of Table 1, below.

As defined herein and described above, R^{b} is hydrogen, or R^{b} connects with the nitrogen where R¹ is attached to form an optionally substituted 5-6 membered partially unsaturated or aromatic heterocyclic ring having 0-2 heteroatoms in addition to the nitrogen atom where R¹ is attached selected from nitrogen, oxygen, and sulfur.

In some embodiments, R^{b} is hydrogen. In some embodiments R connects with R¹ to form an optionally substituted 5-6 membered partially unsaturated or aromatic heterocyclic ring having 0-2 heteroatoms in addition to the nitrogen atom from which R¹ is attached selected from nitrogen, oxygen, and sulfur. In some embodiments, R^{b} connects with R¹ to form an optionally substituted 5-membered partially unsaturated heterocyclic ring having 0-2 heteroatoms in addition to the nitrogen atom from which R¹ is attached selected from nitrogen, oxygen, and sulfur. In some embodiments, R^{b} connects with R¹ to form an optionally substituted 6-membered partially unsaturated heterocyclic ring having 0-2 heteroatoms in addition to the nitrogen atom from which R¹ is attached selected from nitrogen, oxygen, and sulfur. In some embodiments, R connects with R¹ to form an optionally substituted 5-membered aromatic heterocyclic ring having 0-2 heteroatoms in addition to the nitrogen atom from which R¹ is attached selected from nitrogen, oxygen, and sulfur. In some embodiments, R^{b} connects with R¹ to form an optionally substituted 6-membered aromatic heterocyclic ring having 0-2 heteroatoms in addition to the nitrogen atom from which R¹ is attached selected from nitrogen, oxygen, and sulfur. In some embodiments, R^{b} connects with R¹ to form a fused pyrrolidinyl, imidazolyl, piperdinyl, or morpholinyl.

In some embodiments, R^{b} is as depicted in the compounds of Table 1, below.

As defined herein and described above, R^{1a} is R¹, NHR¹, OR¹, or SR¹.

In some embodiments, R^{1a} is R¹. In some embodiments, R^{1a} is NHR¹. In some embodiments, R^{1a} is OR¹. In some embodiments, R^{1a} is SR¹.

As defined herein and described above, R¹ is hydrogen, C₁₋₆ alkyl, C₁₋₆ haloalkyl, C₃₋₆ cycloalkyl, C₃₋₆ heterocycloalkyl, or C₁₋₆ alkyl-O—C₁₋₆ alkyl.

In some embodiments, R¹ is C₃₋₆ heterocycloalkyl. In some embodiments, R¹ is C₁₋₆ alkyl-O—C₁₋₆ alkyl.

In some embodiments, R^{1a} is R¹ and R¹ is C₁₋₆ alkyl, C₁₋₆ haloalkyl or C₃₋₆ cycloalkyl. In some embodiments, R^{1a} is methyl, cyclopropyl, or —CHF.

In some embodiments, R^{1a} is SR¹ and R¹ is C₁₋₆ alkyl. In some embodiments, R^{1a} is —SMe.

In some embodiments, R^{1a} is NHR¹ and R¹ is C₁₋₆ alkyl-O—C₁₋₆ alkyl. In some embodiments, R^{1a} is —NHCH₂CH₂OMe.

In some embodiments, R^{1a} is as depicted in the compounds of Table 1, below.

As defined herein and described above, R¹ is hydrogen, C₁₋₆ alkyl, C₁₋₆ haloalkyl, or C₃₋₆ cycloalkyl.

In some embodiments, R¹ is hydrogen. In some embodiments, R¹ is C₁₋₆ alkyl. In some embodiments, R¹ is C₁₋₆ haloalkyl. In some embodiments, R¹ is C₃₋₆ cycloalkyl.

In some embodiments, R¹ is methyl.

In some embodiments, R¹ is as depicted in the compounds of Table 1, below.

As defined herein and described above, each R², R⁵, R⁶, and R⁷ are independently hydrogen, halogen, —CN, —NO₂, —OR, oxo, —SR, —NR₂, —SiR₃, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, or R^{A}, or two R⁶ on adjacent atoms connect to form a fused optionally substituted 5-6 membered partially saturated or aromatic heterocyclic ring having 1-2 heteroatoms selected from nitrogen, oxygen, and sulfur, or two R² groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

In some embodiments, R² is hydrogen. In some embodiments, R² is halogen. In some embodiments, R² is —CN. In some embodiments, R² is —NO₂. In some embodiments, R² is —OR. In some embodiments, R² is oxo. In some embodiments, R² is —SR. In some embodiments, R² is —NR₂. In some embodiments, R² is —SiR₃. In some embodiments, R² is —S(O)₂R. In some embodiments, R² is —S(O)₂NR₂. In some embodiments, R² is —S(O)R. In some embodiments, R² is —C(O)R. In some embodiments, R² is —C(O)OR. In some embodiments, R² is —C(O)NR₂. In some embodiments, R² is —C(O)N(R)OR. In some embodiments, R² is —OC(O)R. In some embodiments, R² is —OC(O)NR₂. In some embodiments, R² is —OP(O)R₂. In some embodiments, R² is —OP(O)(OR)₂. In some embodiments, R² is —OP(O)(OR)NR₂. In some embodiments, R² is —OP(O)(NR₂)₂. In some embodiments, R² is —N(R)C(O)OR. In some embodiments, R² is —N(R)C(O)R. In some embodiments, R² is —N(R)C(O)NR₂. In some embodiments, R² is —N(R)S(O)₂R. In some embodiments, R² is —N(R)P(O)R₂. In some embodiments, R² is —N(R)P(O)(OR)₂. In some embodiments, R² is —N(R)P(O)(OR)NR₂. In some embodiments, R² is —N(R)P(O)(NR₂)₂. In some embodiments, R² is —N(R)S(O)₂R. In some embodiments, R² is R^{A}.

In some embodiments, two R² groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

In some embodiments, two R² groups of Ring A are taken together with their intervening atoms to form an optionally substituted ring selected from a 3-10 membered saturated or partially unsaturated carbocyclyl. In some embodiments, two R² groups on the same or adjacent atoms are taken together with their intervening atoms to form an optionally substituted ring selected from a 5- to 6-membered saturated or partially unsaturated carbocyclyl.

In some embodiments, two R² groups of Ring A are taken together with their intervening atoms to form an optionally substituted ring selected from a 3-10 membered saturated or partially unsaturated heterocyclyl having 1-4 heteroatoms independently selected from nitrogen, oxygen, or sulfur. In some embodiments, two R² groups of Ring A are taken together with their intervening atoms to form an optionally substituted ring selected from a 4-7 membered saturated or partially unsaturated heterocyclyl having 1-4 heteroatoms independently selected from nitrogen, oxygen, or sulfur. In some embodiments, two R² groups of Ring A are taken together with their intervening atoms to form an optionally substituted ring selected from a 5-6 membered saturated or partially unsaturated heterocyclyl having 1-4 heteroatoms independently selected from nitrogen, oxygen, or sulfur.

In some embodiments, two R² groups of Ring A are taken together with their intervening atoms to form an optionally substituted benzo.

In some embodiments, two R² groups of Ring A are taken together with their intervening atoms to form an optionally substituted ring selected from a 5-10 membered heteroaryl having 1-4 heteroatoms independently selected from nitrogen, oxygen, or sulfur. In some embodiments, two R² groups of Ring A are taken together with their intervening atoms to form an optionally substituted ring selected from a 5- to 6-membered heteroaryl having 1-4 heteroatoms independently selected from nitrogen, oxygen, or sulfur.

In some embodiments, R² is hydrogen, fluoro, chloro, methyl, ethyl, cyclopropyl, —CHF₂, —CF₃, oxo, —OMe, or -OEt. In some embodiments, R² is hydrogen, fluoro, chloro, methyl, ethyl, —CHF₂, or —CF₃. In some embodiments, R² is hydrogen, chloro, or methyl. In some embodiments, R² is chloro and methyl.

In some embodiments, R² is hydrogen. In some embodiments, R² is halogen. In some embodiments, R⁵ is —CN. In some embodiments, R⁵ is —NO₂. In some embodiments, R⁵ is —OR. In some embodiments, R⁵ is oxo. In some embodiments, R⁵ is —SR. In some embodiments, R⁵ is —NR₂. In some embodiments, R⁵ is —SiR₃. In some embodiments, R⁵ is —S(O)₂R. In some embodiments, R⁵ is —S(O)₂NR₂. In some embodiments, R⁵ is —S(O)R. In some embodiments, R⁵ is —C(O)R. In some embodiments, R is —C(O)OR. In some embodiments, R⁵ is —C(O)NR₂. In some embodiments, R⁵ is —C(O)N(R)OR. In some embodiments, R⁵ is —OC(O)R. In some embodiments, R⁵ is —OC(O)NR₂. In some embodiments, R⁵ is —OP(O)R₂. In some embodiments, R⁵ is —OP(O)(OR)₂. In some embodiments, R⁵ is —OP(O)(OR)NR₂. In some embodiments, R⁵ is —OP(O)(NR₂)₂. In some embodiments, R⁵ is —N(R)C(O)OR. In some embodiments, R⁵ is —N(R)C(O)R. In some embodiments, R⁵ is —N(R)C(O)NR₂. In some embodiments, R is —N(R)S(O)₂R. In some embodiments, R⁵ is —N(R)P(O)R₂. In some embodiments, R⁵ is —N(R)P(O)(OR)₂. In some embodiments, R⁵ is —N(R)P(O)(OR)NR₂. In some embodiments, R⁵ is —N(R)P(O)(NR₂)₂. In some embodiments, R⁵ is —N(R)S(O)₂R. In some embodiments, R⁵ is R^{A}.

In some embodiments, R⁵ is hydrogen, fluoro, chloro, methyl, ethyl, cyclopropyl, —CHF₂, —CF₃, oxo, —OMe, -OEt, or an optionally substituted 3-7 membered saturated or partially unsaturated heterocyclic ring having 1-2 heteroatoms independently selected from nitrogen, oxygen, and sulfur.

In some embodiments, R⁵ is

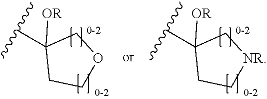

In some embodiments, R⁵ is methyl,

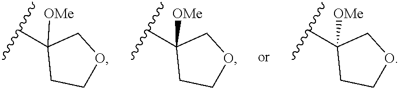 [SMILES: *C1(OC)CCOC1 ; *[C@@]1(OC)CCOC1 ; "or" ; *[C@]1(OC)CCOC1]

In some embodiments, R⁶ is hydrogen. In some embodiments, R⁶ is halogen. In some embodiments, R⁶ is —CN. In some embodiments, R⁶ is —NO₂. In some embodiments, R⁶ is —OR. In some embodiments, R⁶ is oxo. In some embodiments, R⁶ is —SR. In some embodiments, R⁶ is —NR₂. In some embodiments, R⁶ is —SiR₃. In some embodiments, R⁶ is —S(O)₂R. In some embodiments, R⁶ is —S(O)₂NR₂. In some embodiments, R⁶ is —S(O)R. In some embodiments, R⁶ is —C(O)R. In some embodiments, R⁶ is —C(O)OR. In some embodiments, R⁶ is —C(O)NR₂. In some embodiments, R⁶ is —C(O)N(R)OR. In some embodiments, R⁶ is —OC(O)R. In some embodiments, R⁶ is —OC(O)NR₂. In some embodiments, R⁶ is —OP(O)R₂. In some embodiments, R⁶ is —OP(O)(OR)₂. In some embodiments, R⁶ is —OP(O)(OR)NR₂. In some embodiments, R⁶ is —OP(O)(NR₂)₂. In some embodiments, R⁶ is —N(R)C(O)OR. In some embodiments, R⁶ is —N(R)C(O)R. In some embodiments, R⁶ is —N(R)C(O)NR₂. In some embodiments, R⁶ is —N(R)S(O)₂R. In some embodiments, R⁶ is —N(R)P(O)R₂. In some embodiments, R⁶ is —N(R)P(O)(OR)₂. In some embodiments, R⁶ is —N(R)P(O)(OR)NR₂. In some embodiments, R⁶ is —N(R)P(O)(NR₂)₂. In some embodiments, R⁶ is —N(R)S(O)₂R. In some embodiments, R⁶ is R^{A}. In some embodiments, two R⁶ on adjacent atoms connect to form a fused optionally substituted 5-6 membered partially saturated or aromatic heterocyclic ring having 1-2 heteroatoms selected from nitrogen, oxygen, and sulfur. In some embodiments, two Ron adjacent atoms form or

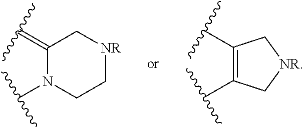

In some embodiments, R⁶ is hydrogen, fluoro, chloro, methyl, ethyl, cyclopropyl, —CHF₂, —CF₃, oxo, —OMe, -OEt, —NHCOMe, or —NHCOEt.

In some embodiments, R⁷ is hydrogen. In some embodiments, R⁷ is halogen. In some embodiments, R⁷ is —CN. In some embodiments, R⁷ is —NO₂. In some embodiments, R⁷ is —OR. In some embodiments, R⁷ is oxo. In some embodiments, R⁷ is —SR. In some embodiments, R⁷ is —NR₂. In some embodiments, R¹ is —SiR₃. In some embodiments, R⁷ is —S(O)₂R. In some embodiments, R⁷ is —S(O)₂NR₂. In some embodiments, R⁷ is —S(O)R. In some embodiments, R⁷ is —C(O)R. In some embodiments, R⁷ is —C(O)OR. In some embodiments, R⁷ is —C(O)NR₂. In some embodiments, R¹ is —C(O)N(R)OR. In some embodiments, R¹ is —OC(O)R. In some embodiments, R¹ is —OC(O)NR₂. In some embodiments, R⁷ is —OP(O)R₂. In some embodiments, R⁷ is —OP(O)(OR)₂. In some embodiments, R¹ is —OP(O)(OR)NR₂. In some embodiments, R¹ is —OP(O)(NR₂)₂. In some embodiments, R⁷ is —N(R)C(O)OR. In some embodiments, R¹ is —N(R)C(O)R. In some embodiments, R¹ is —N(R)C(O)NR₂. In some embodiments, R is —N(R)S(O)₂R. In some embodiments, R⁷ is —N(R)P(O)R₂. In some embodiments, R⁷ is —N(R)P(O)(OR)₂. In some embodiments, R¹ is —N(R)P(O)(OR)NR₂. In some embodiments, R¹ is —N(R)P(O)(NR₂)₂. In some embodiments, R⁷ is —N(R)S(O)₂R. In some embodiments, R⁷ is R^{A}.

In some embodiments, R⁷ is hydrogen, fluoro, chloro, methyl, ethyl, cyclopropyl, —CHF₂, —CF₃, oxo, —OMe, or -OEt.

In some embodiments, R², R⁵, R⁶, and R⁷ are as depicted in the compounds of Table 1, below.

As defined herein and described above, each of m, n, o, and p are independently 0, 1, 2, 3, or 4.

In some embodiments, m is 0. In some embodiments, m is 1. In some embodiments, m is 2. In some embodiments, m is 3. In some embodiments, m is 4.

In some embodiments, m is 0, 1, 2, or 3. In some embodiments, m is 0, 1, or 2. In some embodiments, m is 0 or 1. In some embodiments, m is 1 or 2. In some embodiments, m is 2 or 3.

In some embodiments, n is 0. In some embodiments, n is 1. In some embodiments, n is 2. In some embodiments, n is 3. In some embodiments, n is 4.

In some embodiments, n is 0, 1, 2, or 3. In some embodiments, n is 0, 1, or 2. In some embodiments, n is 0 or 1. In some embodiments, n is 1 or 2. In some embodiments, n is 2 or 3.

In some embodiments, o is 0. In some embodiments, o is 1. In some embodiments, o is 2. In some embodiments, o is 3. In some embodiments, o is 4.

In some embodiments, o is 0, 1, 2, or 3. In some embodiments, o is 0, 1, or 2. In some embodiments, o is 0 or 1. In some embodiments, o is 1 or 2. In some embodiments, o is 2 or 3.

In some embodiments, p is 0. In some embodiments, p is 1. In some embodiments, p is 2. In some embodiments, p is 3. In some embodiments, p is 4.

In some embodiments, p is 0, 1, 2, or 3. In some embodiments, p is 0, 1, or 2. In some embodiments, p is 0 or 1. In some embodiments, p is 1 or 2. In some embodiments, p is 2 or 3.

In some embodiments, m, n, o, and p are as depicted in the compounds of Table 1, below.

As defined herein and described above, R³ is hydrogen, or an optionally substituted group selected from C₁₋₆ aliphatic, phenyl, a 3-11 membered saturated or partially unsaturated monocyclic, bicyclic, bridged bicyclic, or spirocyclic carbocyclylenyl or heterocyclylenyl having 1-3 heteroatoms independently selected from nitrogen, oxygen, and sulfur, and a 5-10 membered monocyclic or bicyclic heteroaryl having 1-3 heteroatoms independently selected from nitrogen, oxygen, and sulfur.

In some embodiments, R³ is an optionally substituted C₁₋₆ aliphatic. In some embodiments, R³ is an optionally substituted phenyl. In some embodiments, R³ is an optionally substituted 3-11 membered saturated or partially unsaturated monocyclic, bicyclic, bridged bicyclic, or spirocyclic carbocyclylenyl. In some embodiments, R³ is an optionally substituted 3-11 membered saturated or partially unsaturated monocyclic, bicyclic, bridged bicyclic, or spirocyclic carbocyclylenyl or heterocyclylenyl having 1-3 heteroatoms independently selected from nitrogen, oxygen, and sulfur. In some embodiments, R³ is an optionally substituted 5-10 membered monocyclic or bicyclic heteroaryl having 1-3 heteroatoms independently selected from nitrogen, oxygen, and sulfur.

As defined herein and described above, R³ is hydrogen, or a C₁₋₆ alkyl or C₃₋₆ cycloalkyl optionally substituted with 1-2 substituents selected from halogen, —CN, C₁₋₆ alkyl, and —OR, or R³ and R⁵ are joined by a bivalent, saturated or partially unsaturated, straight or branched C₃₋₆ hydrocarbon chain, wherein 0-2 methylene units of the chain are independently replaced by —CR₂—, —CRF—, —CF₂—, —O—, —NR—, —C(O)—.

In some embodiments, R³ is hydrogen. In some embodiments, R³ is C₁₋₆ alkyl. In some embodiments, R³ is C₁₋₆ alkyl substituted with 1-2 substituents selected from halogen, —CN, and —OR. In some embodiments, R³ is C₃₋₆ cycloalkyl. In some embodiments, R³ is C₃₋₆ cycloalkyl substituted with 1-2 substituents selected from halogen. —CN, C₁₋₆ alkyl, C₁₋₆haloalkyl, and —OR. In some embodiments, R³ is C₄₋₆ cycloalkyl. In some embodiments, R³ is C₄₋₆ cycloalkyl substituted with 1-2 substituents selected from halogen, —CN, C₁₋₆ alkyl, C₁₋₆ haloalkyl, and —OR. In some embodiments, R³ is C₄₋₅ cycloalkyl. In some embodiments, R³ is C₄₋₅ cycloalkyl substituted with 1-2 substituents selected from halogen, —CN, C₁₋₆ alkyl, C₁₋₆ haloalkyl, and —OR. In some embodiments, R³ is C₄ cycloalkyl. In some embodiments, R³ is C₄ cycloalkyl substituted with 1-2 substituents selected from halogen, —CN, C₁₋₆ alkyl, C₁₋₆ haloalkyl, and —OR. In some embodiments, R³ is C₄₋₆ cycloalkyl substituted with —OR. In some embodiments, R³ is C₄₋₅ cycloalkyl substituted with —OR. In some embodiments, R³ is C₄ cycloalkyl substituted with —OR. In some embodiments, R³ is C₄₋₆ cycloalkyl substituted with —OC₁₋₆ alkyl. In some embodiments, R³ is C₄₋₅ cycloalkyl substituted with —OC₁₋₆ alkyl. In some embodiments, R³ is C₄ cycloalkyl substituted with —OC₁₋₆ alkyl. In some embodiments, R³ is C₄₋₆ cycloalkyl substituted with —OMe. In some embodiments, R³ is C₄₋₅ cycloalkyl substituted with —OMe. In some embodiments, R³ is C₄ cycloalkyl substituted with —OMe. In some embodiments, R³ and R⁵ are joined by a bivalent, saturated or partially unsaturated, straight or branched C₃₋₆ hydrocarbon chain, wherein 0-2 methylene units of the chain are independently replaced by —CR₂—, —CRF—, —CF₂—, —O—, —NR—, —C(O)—.

As defined herein and described above, R³ is hydrogen, or a C₁₋₆ alkyl or 3-9 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur optionally substituted with 1-2 substituents selected from halogen, —CN, C₁₋₆ alkyl, C₁₋₆ haloalkyl, —OR, —CH₂OR, or a 5-membered heteroaryl having 1-3 heteroatoms independently selected from nitrogen, oxygen, and sulfur.

In some embodiments, R³ is C₁₋₆ alkyl. In some embodiments, R³ is C₁₋₆ alkyl substituted with 1-2 substituents selected from halogen, —CN, C₁₋₆ alkyl, C₁₋₆ haloalkyl, —OR, —CH₂OR, or a 5-membered heteroaryl having 1-3 heteroatoms independently selected from nitrogen, oxygen, and sulfur.

In some embodiments, R³ is a 3-9 membered saturated or partially unsaturated monocyclic, bicyclic, bridged bicyclic, or spirocyclic carbocyclic ring. In some embodiments, R³ is a 3-9 membered saturated or partially unsaturated monocyclic, bicyclic, bridged bicyclic, or spirocyclic carbocyclic ring substituted with 1-2 substituents selected from halogen, —CN, C₁₋₆ alkyl, C₁₋₆ haloalkyl, —OR, —CH₂OR, or a 5-membered heteroaryl having 1-3 heteroatoms independently selected from nitrogen, oxygen, and sulfur.

In some embodiments, R³ is a 4-9 membered saturated or partially unsaturated monocyclic, bicyclic, bridged bicyclic, or spirocyclic carbocyclic ring. In some embodiments, R³ is a 4-9 membered saturated or partially unsaturated monocyclic, bicyclic, bridged bicyclic, or spirocyclic carbocyclic ring substituted with 1-2 substituents selected from halogen, —CN, C₁₋₆ alkyl, C₁₋₆ haloalkyl, —OR, —CH₂OR, or a 5-membered heteroaryl having 1-3 heteroatoms independently selected from nitrogen, oxygen, and sulfur.

In some embodiments, R³ is a 3-9 membered saturated or partially unsaturated monocyclic, bicyclic, bridged bicyclic, or spirocyclic heterocyclic ring having 1-3 heteroatoms independently selected from nitrogen, oxygen, and sulfur. In some embodiments, R³ is a 3-9 membered saturated or partially unsaturated monocyclic, bicyclic, bridged bicyclic, or spirocyclic heterocyclic ring having 1-3 heteroatoms independently selected from nitrogen, oxygen, and sulfur substituted with 1-2 substituents selected from halogen, —CN, C₁₋₆ alkyl, C₁₋₆ haloalkyl, —OR, —CH₂OR, or a 5-membered heteroaryl having 1-3 heteroatoms independently selected from nitrogen, oxygen, and sulfur.

In some embodiments, R³ is a 3-5 or 7-9 membered saturated monocyclic, a 3-9 membered partially unsaturated monocyclic, or a 3-9 membered saturated or partially unsaturated bicyclic, bridged bicyclic, or spirocyclic heterocyclic ring having 1-3 heteroatoms independently selected from nitrogen, oxygen, and sulfur. In some embodiments, R³ is a 3-5 or 7-9 membered saturated monocyclic, a 3-9 membered partially unsaturated monocyclic, or a 3-9 membered saturated or partially unsaturated bicyclic, bridged bicyclic, or spirocyclic heterocyclic ring having 1-3 heteroatoms independently selected from nitrogen, oxygen, and sulfur substituted with 1-2 substituents selected from halogen, —CN, C₁₋₆alkyl, C₁₋₆haloalkyl, —OR, —CH₂OR, or a 5-membered heteroaryl having 1-3 heteroatoms independently selected from nitrogen, oxygen, and sulfur.

In some embodiments, R³ is C₁₋₆ alkyl substituted with a 5-membered heteroaryl having 1-3 heteroatoms independently selected from nitrogen, oxygen, and sulfur. In some embodiments, R³ is a 3-6 membered saturated monocyclic or bridged bicyclic carbocyclic ring optionally substituted with 1-2 substituents selected from halogen. —CN, C₁₋₆ alkyl, C₁₋₆ haloalkyl, —OR, —CH₂OR. In some embodiments, R³ is a 7-8 membered saturated or partially unsaturated bicyclic heterocyclic ring having 1-3 heteroatoms independently selected from nitrogen, oxygen, and sulfur substituted with 1-2 substituents selected from halogen, —CN, C₁₋₆ alkyl, C₁₋₆haloalkyl, —OR, —CH₂OR.

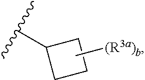

In some embodiments, R³ is, wherein:
each R^{3a} is independently hydrogen, halogen, —CN, —NO₂. —OR, oxo, —SR, —NR₂, —SiR₃, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —P(O)R₂, —P(O)(OR)₂, —P(O)(OR)NR₂, —P(O)(NR₂)₂, or R^{A}; or:
   two R^{3a} groups on the same carbon atom or adjacent carbon atoms are optionally taken together with their intervening atoms to form a 3-9 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur; and
b is 0, 1, 2, 3, 4, or 5.

In some embodiments, R^{3a} is hydrogen. In some embodiments, R^{3a} is halogen. In some embodiments, R^{3a} is —CN. In some embodiments, R^{3a} is —NO₂. In some embodiments, R^{3a} is —OR. In some embodiments, R^{3a} is oxo. In some embodiments, R^{3a} is —SR. In some embodiments, R^{3a} is —NR₂. In some embodiments, R^{3a} is —SiR₃. In some embodiments, R^{3a} is —S(O)₂R, —S(O)₂NR₂. In some embodiments, R^{3a} is —S(O)R. In some embodiments, R^{3a} is —C(O)R. In some embodiments, R^{3a} is —C(O)OR. In some embodiments, R^{3a} is —C(O)NR₂. In some embodiments, R^{3a} is —C(O)N(R)OR. In some embodiments, R^{3a} is —OC(O)R. In some embodiments, R^{3a} is —OC(O)NR₂, —OP(O)R₂. In some embodiments, R^{3a} is —OP(O)(OR)₂. In some embodiments, R^{3a} is —OP(O)(OR)NR₂. In some embodiments, R^{3a} is —OP(O)(NR₂)₂. In some embodiments, R^{3a} is —N(R)C(O)OR. In some embodiments, R^{3a} is —N(R)C(O)R, —N(R)C(O)NR₂. In some embodiments, R^{3a} is —N(R)S(O)₂R. In some embodiments, R^{3a} is —N(R)P(O)R₂. In some embodiments, R^{3a} is —N(R)P(O)(OR)₂. In some embodiments, R^{3a} is —N(R)P(O)(OR)NR₂. In some embodiments, R^{3a} is —N(R)P(O)(NR₂)₂. In some embodiments, R^{3a} is —N(R)S(O)₂R. In some embodiments, R^{3a} is —P(O)R₂. In some embodiments, R^{3a} is —P(O)(OR)₂. In some embodiments, R^{3a} is —P(O)(OR)NR₂. In some embodiments, R^{3a} is —P(O)(NR₂)₂. In some embodiments, R^{3a} is R^{A}. In some embodiments, two R^{3a} groups on the same carbon atom or adjacent carbon atoms are optionally taken together with their intervening atoms to form a 3-9 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur.

In some embodiments, R^{3a} is halogen, —CN, C₁₋₆ alkyl, C₁₋₆ haloalkyl, —OR, —CH₂OR, or two R^{3a} groups on the same carbon atom or adjacent carbon atoms are taken together with their intervening atoms to form a 3-5 membered saturated or partially unsaturated monocyclic carbocyclic or heterocyclic ring having 0-2 heteroatoms independently selected from nitrogen, oxygen, and sulfur.

In some embodiments, R^{3a} is fluoro, chloro, —CN, methyl, ethyl, —OMe, -OEt, —CH₂OMe, —CH₂OEt, or two R^{3a} groups on adjacent carbon atoms are taken together with their intervening atoms to form a 5-membered saturated or partially unsaturated monocyclic heterocyclic ring having 1 heteroatom independently selected from nitrogen, oxygen, and sulfur.

In some embodiments, b is 0. In some embodiments, b is 1. In some embodiments, b is 2. In some embodiments, b is 3. In some embodiments, b is 4. In some embodiments, b is 5. In some embodiments,
b is 1 or 2. In some embodiments, b is 1, 2, or 3. In some embodiments, b is 2 or 3.

In some embodiments, R³ is hydrogen, methyl, ethyl, isopropyl,

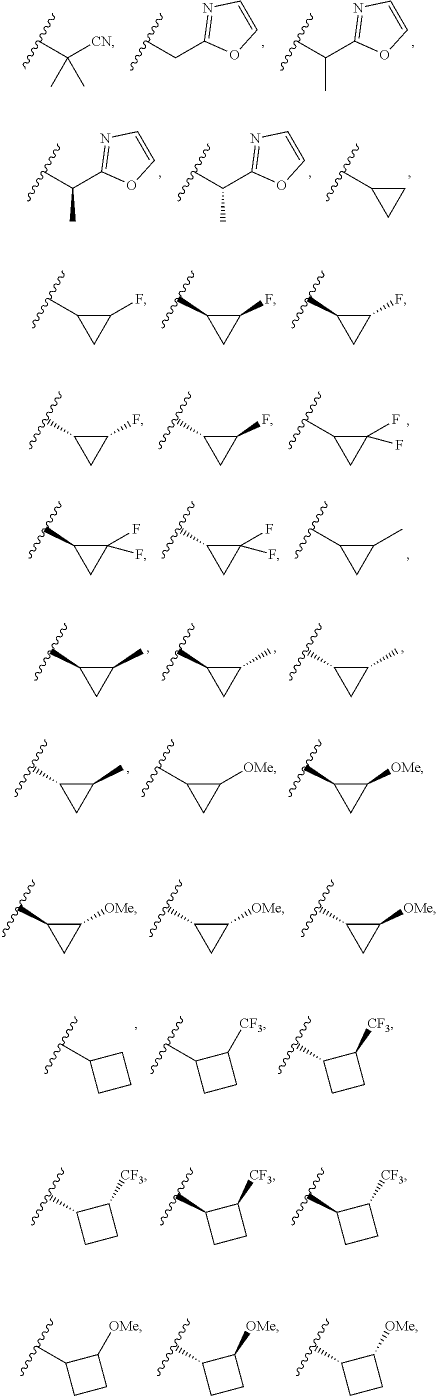

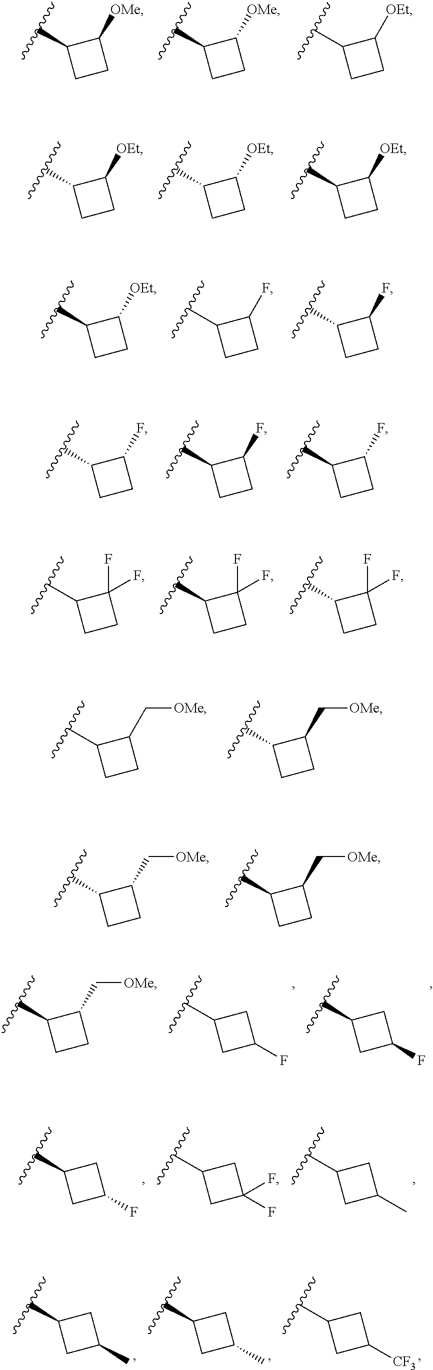

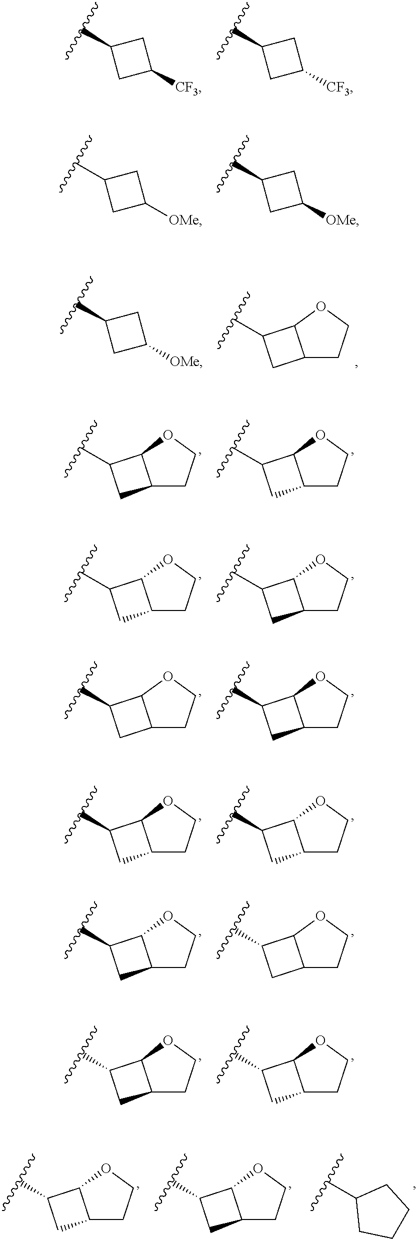

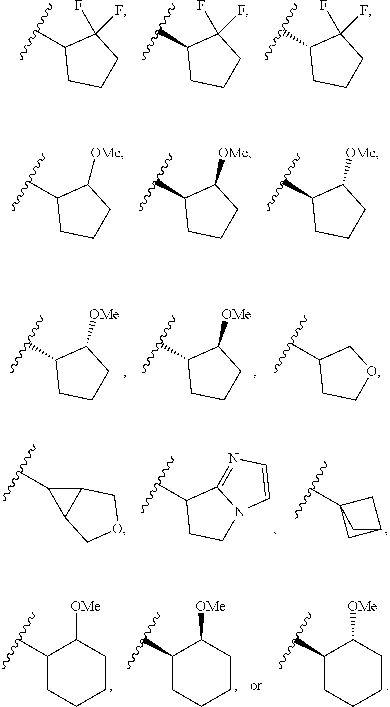

In some embodiments, R³ is as depicted in the compounds of Table 1, below.

As defined herein and described above, R⁴ is hydrogen, halogen, C₁₋₆ alkyl, C₁₋₆ haloalkyl, C₃₋₆ cycloalkyl, —OC₁₋₆ alkyl, or —OC₁₋₆haloalkyl.

In some embodiments, R⁴ is hydrogen. In some embodiments, R⁴ is halogen. In some embodiments, R⁴ is C₁₋₆ alkyl. In some embodiments, R⁴ is C₁₋₆ haloalkyl. In some embodiments, R⁴ is C₃-6 cycloalkyl. In some embodiments, R⁴ is —OC₁₋₆ alkyl. In some embodiments, R⁴ is —OC₁₋₆ haloalkyl.

In some embodiments, R⁴ is fluoro, chloro, methyl, oxo, or —OMe.

In some embodiments, R⁴ is as depicted in the compounds of Table 1, below.

As defined herein and described above, L is a covalent bond, —CR₂—, —CRF—, —CF₂—, —O—, —NR—, —C(O)—, or a bivalent, saturated or partially unsaturated, straight or branched C₁₋₁₀ hydrocarbon chain, wherein 0-6 methylene units of L are independently replaced by -Cy-, —CR₂—, —CRF—, —CF₂—, —CR(OR)—, —O—, —NR—, —C(O)—.

In some embodiments, L is a covalent bond. In some embodiments, L is —CR₂—. In some embodiments, L is —CRF—. In some embodiments, L is —CF₂—. In some embodiments, L is —O—. In some embodiments, L is —NR—. In some embodiments, L is —C(O)—. In some embodiments, L is a bivalent, saturated or partially unsaturated, straight or branched C₁₋₁₀ hydrocarbon chain, wherein 0-6 methylene units of L are independently replaced by -Cy-, —CR₂—, —CRF—, —CF₂—, —CR(OR)—, —O—, —NR—, —C(O)—.

In some embodiments, L is a bivalent, saturated or partially unsaturated, straight or branched C₁₋₁₀ hydrocarbon chain, wherein 0-6 methylene units of L are independently replaced by -Cy-, —CR₂—, —CRF—, —CF₂—, —CR(OR)—, —O—, —NR—, —C(O)—, or S(O)₂.

In some embodiments, L is a bivalent, saturated or partially unsaturated, straight or branched C₁₋₅ hydrocarbon chain, wherein 0-6 methylene units of L are independently replaced by -Cy-, —CR₂—, —CRF—, —CF₂—, —CR(OR)—, —O—, —NR—, —C(O)—, or S(O)₂.

In some embodiments, L is a bivalent, saturated or partially unsaturated, straight or branched C₁₋₅ hydrocarbon chain, wherein 0-6 methylene units of L are independently replaced by -Cy-, —CR₂—, —CRF—, —CF₂—, —CR(OR)—, —O—, —NR—, —C(O)—.

In some embodiments, L is a bivalent, saturated or partially unsaturated, straight or branched C₅ hydrocarbon chain, wherein 0-6 methylene units of L are independently replaced by -Cy-, —CR₂—, —CRF—, —CF₂—, —CR(OR)—, —O—, —NR—, —C(O)—.

In some embodiments, L comprises one -Cy- group.

In some embodiments, L comprises two -Cy- groups.

In some embodiments, L comprises 1-3 methylenes, —C(O)—, a 4-7 membered saturated or partially unsaturated heterocyclylenyl having 1-2 heteroatoms independently selected from nitrogen, oxygen, and sulfur, optionally substituted with 1-2 halogens, and a 6-11 membered saturated or partially unsaturated spirocyclic or bridged bicyclic heterocyclylenyl having 1-2 heteroatoms independently selected from nitrogen, oxygen, and sulfur, optionally substituted with 1-2 halogens.

In some embodiments, L is —O—(C₁₋₅ aliphatic)-. In some embodiments, L is -Cy-(C₁₋₅ aliphatic)-. In some embodiments, L is —(CH₂)₁₋₅-Cy-(C₁₋₅ aliphatic)-. In some embodiments, L is —(CH₂)₁₋₅-Cy-O—(C₁₋₅ aliphatic)-. In some embodiments, L is —(CH₂)₁₋₅-Cy-Cy-(C₁₋₅ aliphatic)-. In some embodiments, L is —(CH₂)₁₋₅-Cy-Cy-O—(C₁₋₅ aliphatic)-.

In some embodiments, L is —(CH₂)₁₋₅-Cy-. In some embodiments, L is —(CH₂)₁₋₅-Cy-CH(OR)—. In some embodiments, L is —(CH₂)₁₋₅-Cy-(CH₂)₁₋₅—. In some embodiments, L is —O—(CH₂)₁₋₅-Cy-. In some embodiments, L is —O—(CH₂)₁₋₅-Cy-(CH₂)₁₋₅—. In some embodiments, L is —(CH₂)₁₋₅—O-Cy-. In some embodiments, L is —(CH₂)₁₋₅—O-Cy-(CH₂)₁₋₅—. In some embodiments, L is —(CH₂)₁₋₅—CO-Cy-(CH₂)₁₋₅—. In some embodiments, L is —(CH₂)₁₋₅—NR-Cy-. In some embodiments, L is —(CH₂)₁₋₅—NR-Cy-CO—. In some embodiments, L is —(CH₂)₁₋₅-Cy-NR—. In some embodiments, L is —O-Cy-NR—. In some embodiments, L is —(CH₂)₁₋₅—NR-Cy-(CH₂)₁₋₅—. In some embodiments, L is —(CH₂)₁₋₅—CO—. In some embodiments, L is —(CH₂)₁ s-Cy-CO—. In some embodiments, L is -Cy-CO—. In some embodiments, L is —O-Cy-CO—. In some embodiments, L is —O-Cy-(CH₂)₁₋₅—. In some embodiments, L is —(CH₂)₁₋₅-Cy-CHF—. In some embodiments, L is —(CH₂)₁₋₅-Cy-CF₂—. In some embodiments, L is —(CH₂)₁₋₅-Cy-(CH₂)₁₋₅—CF₂—.

In some embodiments, L is -Cy-Cy-. In some embodiments, L is —(CH₂)₁₋₅-Cy-Cy-. In some embodiments, L is —(CH₂)₁₋₅-Cy-Cy-CO—. In some embodiments, L is -Cy-(CH₂)₁₋₅—CV—. In some embodiments, L is -Cy-(CH₂)₁₋₅-Cy-CO—. In some embodiments, L is —(CH₂)₁₋₅-Cy-(CH₂)₁₋₅-Cy-CO—. In some embodiments, L is -Cy-(CH₂)₁₋₅-Cy-(CH₂)₁₋₅—. In some embodiments, L is —(CH₂)₁₋₅-Cy-Cy-(CH₂)₁₋₅—. In some embodiments, L is —O—(CH₂)₁₋₅-Cy-Cy-. In some embodiments, L is —O—(CH₂)₁₋₅-Cy-Cy-(CH₂)₁₋₅—. In some embodiments, L is —(CH₂)₁₋₅—O-Cy-Cy-. In some embodiments, L is —(CH₂)₁₋₅—O-Cy-Cy-(CH₂)₁₋₅—. In some embodiments, L is —(CH₂)₁₋₅—NR-Cy-Cy-. In some embodiments, L is —(CH₂)₁₋₅-Cy-NR-Cy-. In some embodiments, L is —(CH₂)₁₋₅—NR-Cy-Cy-(CH₂)₁₋₅—. In some embodiments, L is —(CH₂)₁₋₅-Cy-Cy-CO—. In some embodiments, L is —O-Cy-Cy-CO—. In some embodiments, L is —O-Cy-Cy-(CH₂)₁₋₅—.

In some embodiments, L is -Cy-Cy-Cy-. In some embodiments, L is -Cy-Cy-Cy-CO—. In some embodiments, L is —(CH₂)₁₋₅-Cy-Cy-Cy-. In some embodiments, L is —(CH₂)₁₋₅-Cy-Cy-Cy-CO—. In some embodiments, L is -Cy-(CH₂)₁₋₅-Cy-Cy-. In some embodiments, L is -Cy-(CH₂)₁₋₅-Cy-Cy-CO—. In some embodiments, L is -Cy-(CH₂)₁₋₅-Cy-(CH₂)₁₋₅-Cy-. In some embodiments, L is -Cy-(CH₂)₁₋₅-Cy-(CH₂)₁₋₅-Cy-CO.

As defined herein and described above, each -Cy- is independently an optionally substituted bivalent ring selected from phenylenyl, 8-10 membered bicyclic arylenyl, 4-7 membered saturated or partially unsaturated carbocyclylenyl, 6-11 membered saturated or partially unsaturated spirocyclic or bridged bicyclic carbocyclylenyl, 8-10 membered bicyclic saturated or partially unsaturated carbocyclylenyl, 4-7 membered saturated or partially unsaturated heterocyclylenyl having 1-2 heteroatoms independently selected from nitrogen, oxygen, and sulfur, 6-11 membered saturated or partially unsaturated spirocyclic or bridged bicyclic heterocyclylenyl having 1-2 heteroatoms independently selected from nitrogen, oxygen, and sulfur, 8-10 membered bicyclic saturated or partially unsaturated heterocyclylenyl having 1-2 heteroatoms independently selected from nitrogen, oxygen, and sulfur, 5-6 membered heteroarylenyl having 1-4 heteroatoms independently selected from nitrogen, oxygen, and sulfur, and 8-10 membered bicyclic heteroarylenyl having 1-5 heteroatoms independently selected from nitrogen, oxygen, and sulfur.

In some embodiments, -Cy- is an optionally substituted phenylenyl. In some embodiments, -Cy- is an optionally substituted 8-10 membered bicyclic arylenyl. In some embodiments, -Cy- is an optionally substituted 4-7 membered saturated or partially unsaturated carbocyclylenyl. In some embodiments, -Cy- is an optionally substituted 6-11 membered saturated or partially unsaturated spirocyclic bicyclic carbocyclylenyl. In some embodiments, -Cy- is an optionally substituted 6-11 membered saturated or partially unsaturated bridged bicyclic carbocyclylenyl. In some embodiments, -Cy- is an optionally substituted 8-10 membered bicyclic saturated or partially unsaturated carbocyclylenyl. In some embodiments, -Cy- is an optionally substituted 4-7 membered saturated or partially unsaturated heterocyclylenyl having 1-2 heteroatoms independently selected from nitrogen, oxygen, and sulfur. In some embodiments, -Cy- is an optionally substituted 6-11 membered saturated or partially unsaturated spirocyclic heterocyclylenyl having 1-2 heteroatoms independently selected from nitrogen, oxygen, and sulfur. In some embodiments, -Cy- is an optionally substituted 6-11 membered saturated or partially unsaturated bridged bicyclic heterocyclylenyl having 1-2 heteroatoms independently selected from nitrogen, oxygen, and sulfur. In some embodiments, -Cy- is an optionally substituted 8-10 membered bicyclic saturated or partially unsaturated heterocyclylenyl having 1-2 heteroatoms independently selected from nitrogen, oxygen, and sulfur. In some embodiments, -Cy- is an optionally substituted 5-6 membered heteroarylenyl having 1-4 heteroatoms independently selected from nitrogen, oxygen, and sulfur. In some embodiments, -Cy- is an optionally substituted 8-10 membered bicyclic heteroarylenyl having 1-5 heteroatoms independently selected from nitrogen, oxygen, and sulfur.

In some embodiment, -Cy- is unsubstituted. In some embodiment, -Cy- is substituted. In some embodiments, -Cy- is substituted with halogen, C₁₋₆ alkyl, C₁₋₆ haloalkyl, C₃₋₆ cycloalkyl, —OH, —OC₁₋₆ alkyl, or —OC₁₋₆ haloalkyl. In some embodiments, -Cy- is substituted with fluoro, geminal difluoro, methyl, geminal dimethyl, —OH, —CHF₂, or —CF₃. In some embodiments, one -Cy- group of L is substituted. In some embodiments, two -Cy- groups of L are substituted. In some embodiments, one -Cy- group of L is substituted with 1-2 halogens. In some embodiments, two -Cy- groups of L is substituted with 1-2 halogens. In some, embodiments. -Cy- is substituted with geminal difluoro. In some embodiments, one -Cy- group of L is substituted with geminal difluoro. In some embodiments, two -Cy- groups of L is substituted with geminal difluoro.

In some embodiments, L is —(CR₂)ₐ-Cy-C(O)—, wherein -Cy- is

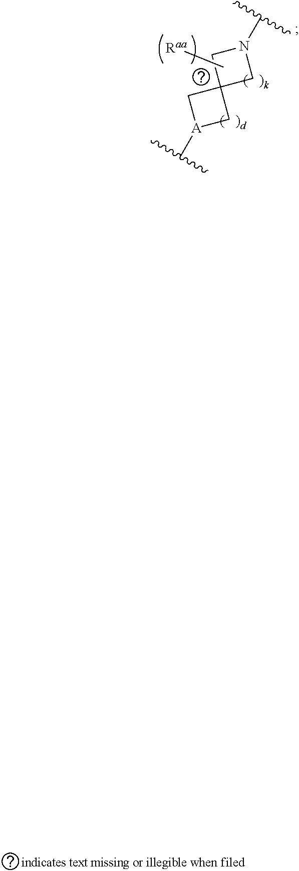

wherein:
A is —N— or —CH—;
each R^{aa} is halogen;
each R₂ is independently H, C₁₋₃ alkyl, —O—, or —N(CH₃)—;
a is an integer from 0 to 3;
d is an integer from 1 to 3;
e is an integer from 0 to 2; and
k is an integer from 1 to 3.

In some embodiments, A is N. In some embodiments, A is —CH—. In some embodiments, R₂ is H. In some embodiments, a is 0. In some embodiments, a is 1. In some embodiments, a is 2. In some embodiments, a is 3. In some embodiments, d is 1. In some embodiments, d is 2. In some embodiments, d is 3. In some embodiments, each R^{aa} is fluoro. In some embodiments, e is 0. In some embodiments, e is 1. In some embodiments, each e is 2. In some embodiments, k is 1. In some embodiments, k is 2. In some embodiments, k is 3. In some embodiments, -Cy- is:

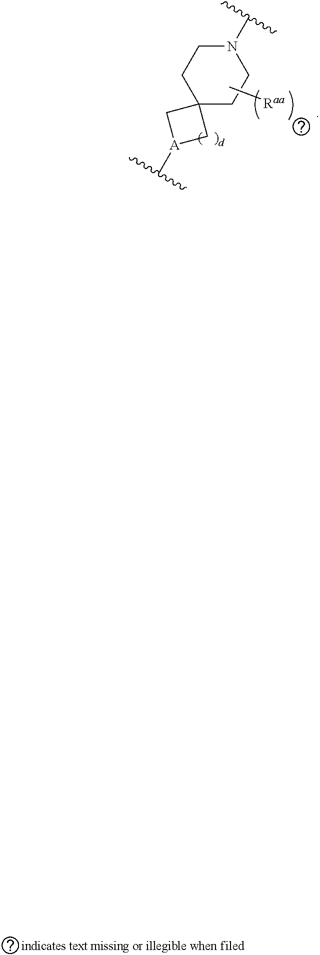

In some embodiments, -Cy- is:

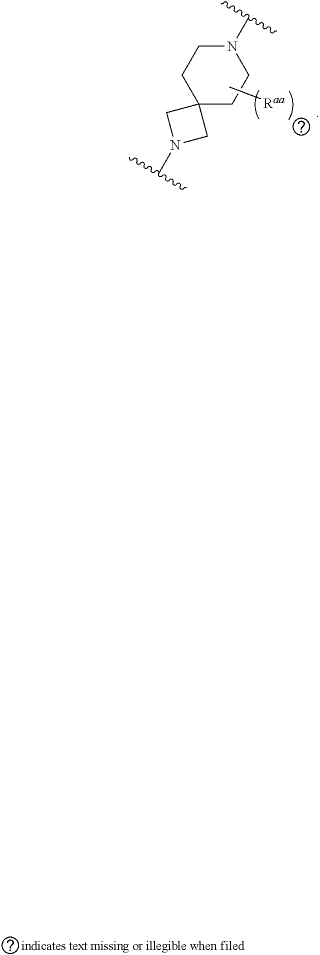

In some embodiments, -Cy- is:

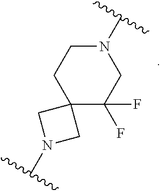 [SMILES: *N1CCC2(CN(*)C2)C(F)(F)C1]

In some embodiments, L is:

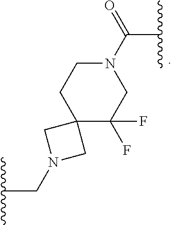 [SMILES: *CN1CC2(CCN(C(*)=O)CC2(F)F)C1]

In some embodiments, L is —(CR2)ₐ-Cy-Cy-(CR₂)b-Cy-C(O)—, wherein each -Cy- is independently

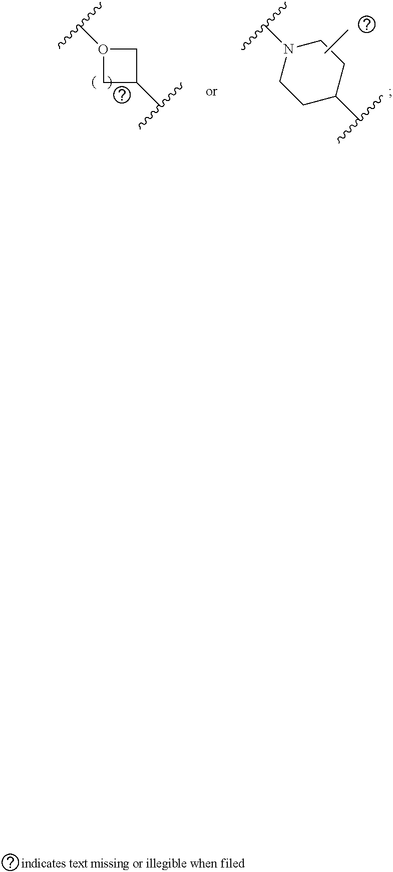

wherein:
D is —N— or —CH—;
each R^{bb} is halogen;
each R₂ is independently H C₁₋₃ alkyl, —O—, or —N(CH₃)—;
a is 0 or 1;
b is 0 or 1;
g is an integer from 1 to 3; and
h is an integer from 0 to 2.

In some embodiments, D is N. In some embodiments, D is —CH—. In some embodiments, each R^{bb} is fluoro. In some embodiments, each R₂ is H. In some embodiments, g is 1. In some embodiments, g is 2. In some embodiments, g is 3. In some embodiments, h is 0. In some embodiments, h is 1. In some embodiments, h is 2.

In some embodiments, L is —(CR₂)ₐ-Cy-(CR₂)b-Cy-C(O)—, wherein each -Cy- is independently

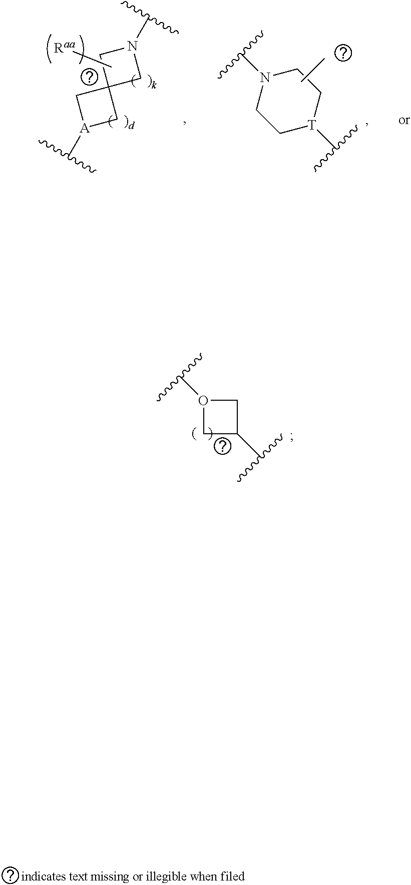

wherein:
A is —N— or —CH—;
D is —N— or —CH—;
T is —N— or —CH—;
each R^{aa} is halogen;
each R^{bb} is halogen;
each R₂ is independently H, C₁₋₃ alkyl, —O—, or —N(CH₃)—;
a is 0 or 1;
b is 0 or 1;
d is an integer from 1 to 3;
e is an integer from 0 to 2;
g is an integer from 1 to 3;
j is an integer from 0 to 2; and
k is an integer from 1 to 3.

In some embodiments, A is —N—. In some embodiments, A is —CH—. In some embodiments, T is —N—. In some embodiments, T is —CH—. In some embodiments, each R^{aa} is fluoro. In some embodiments, each R^{b} is fluoro. In some embodiments, each R² is H. In some embodiments a is 0. In some embodiments, a is 1. In some embodiments, b is 0. In some embodiments, b is 1. In some embodiments, d is 1. In some embodiments, d is 2. In some embodiments, d is 3. In some embodiments, h is 0. In some embodiments, e is 0. In some embodiments, e is 1. In some embodiments, e is 2. In some embodiments, g is 1. In some embodiments, g is 2. In some embodiments, g is 3. In some embodiments, j is 0. In some embodiments, j is 1. In some embodiments, j is 2. In some embodiments, k is 1. In some embodiments, k is 2. In some embodiments, k is 3.

In some embodiments, each -Cy- is independently

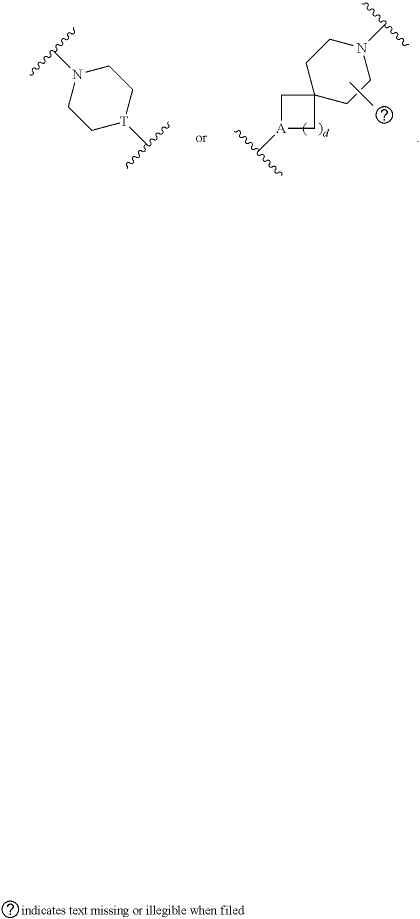

In some embodiments, each -Cy- is independently

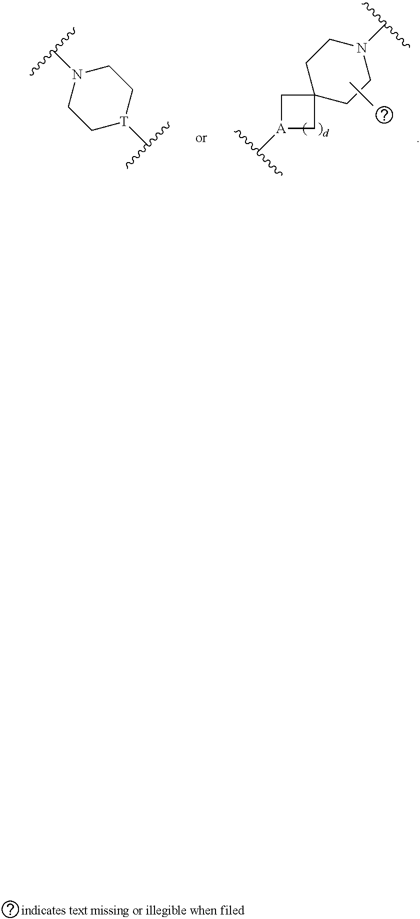

In some embodiments, each -Cy- is independently

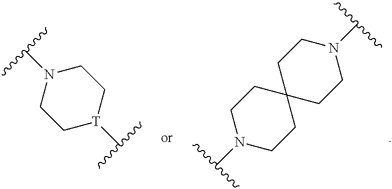

In some embodiments, each -Cy- is independently

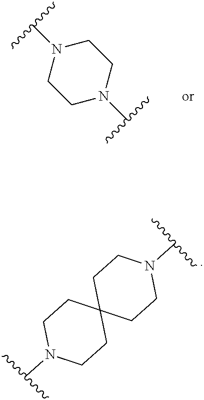 [SMILES: *N1CCN(*)CC1 ; *N1CCC2(CC1)CCN(*)CC2]

In some embodiments, L is

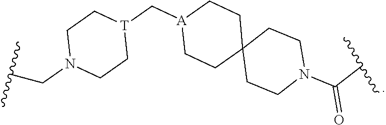

In some embodiments, L is

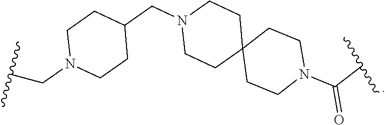 [SMILES: *CN1CCC(CN2CCC3(CC2)CCN(C(*)=O)CC3)CC1]

In some embodiments, -Cy- is

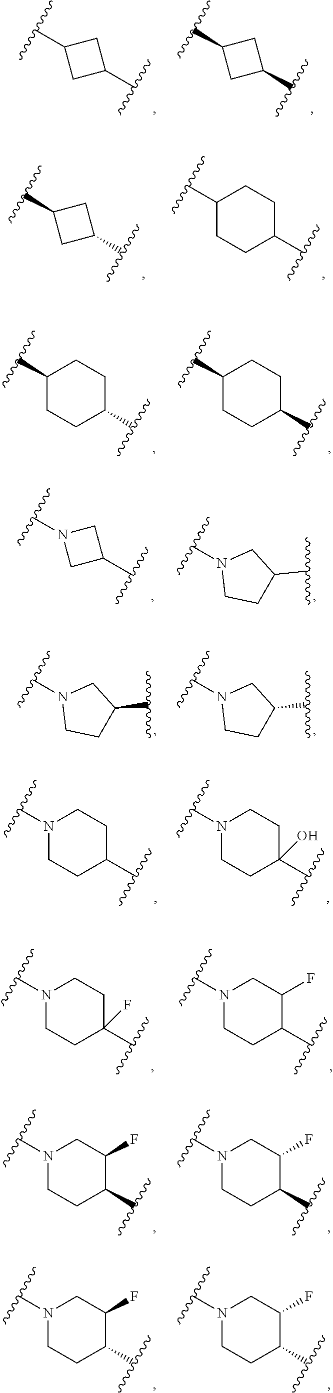

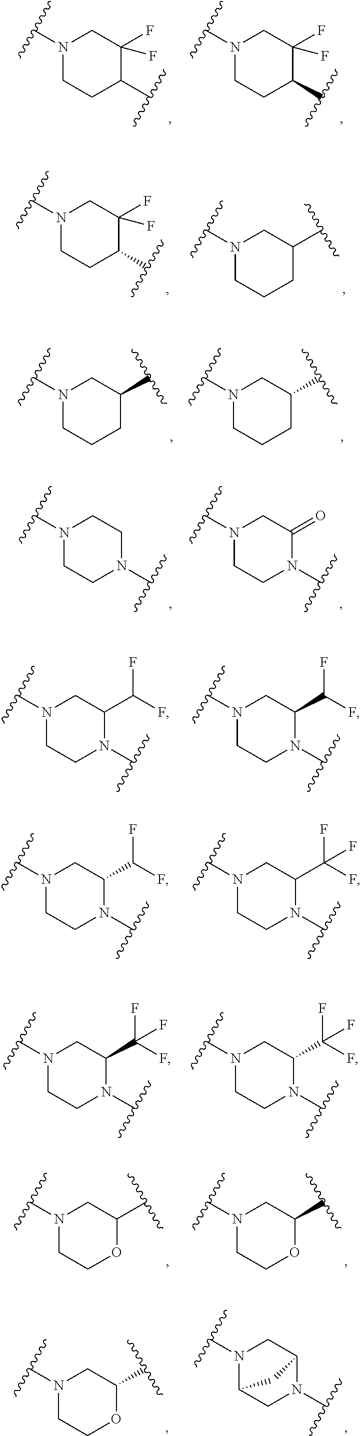

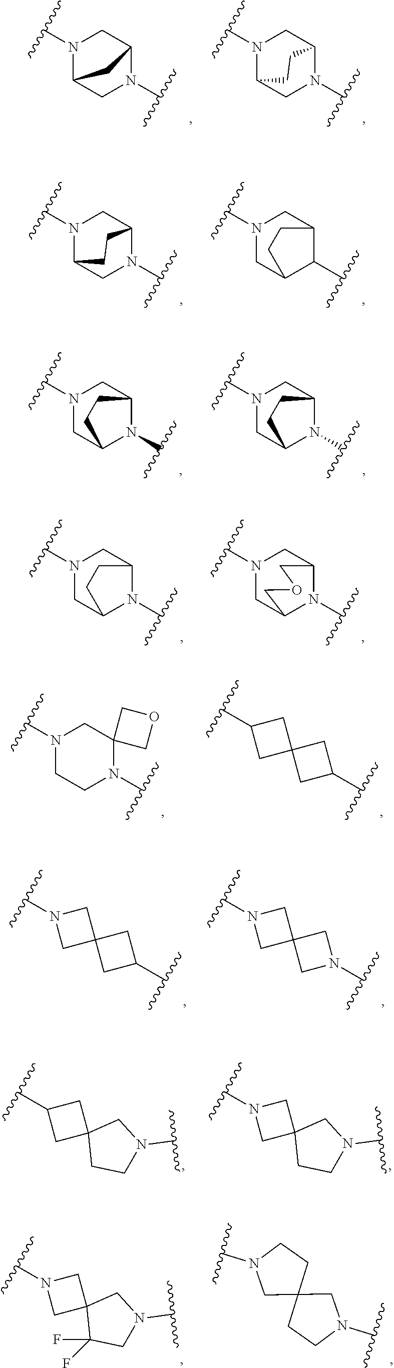

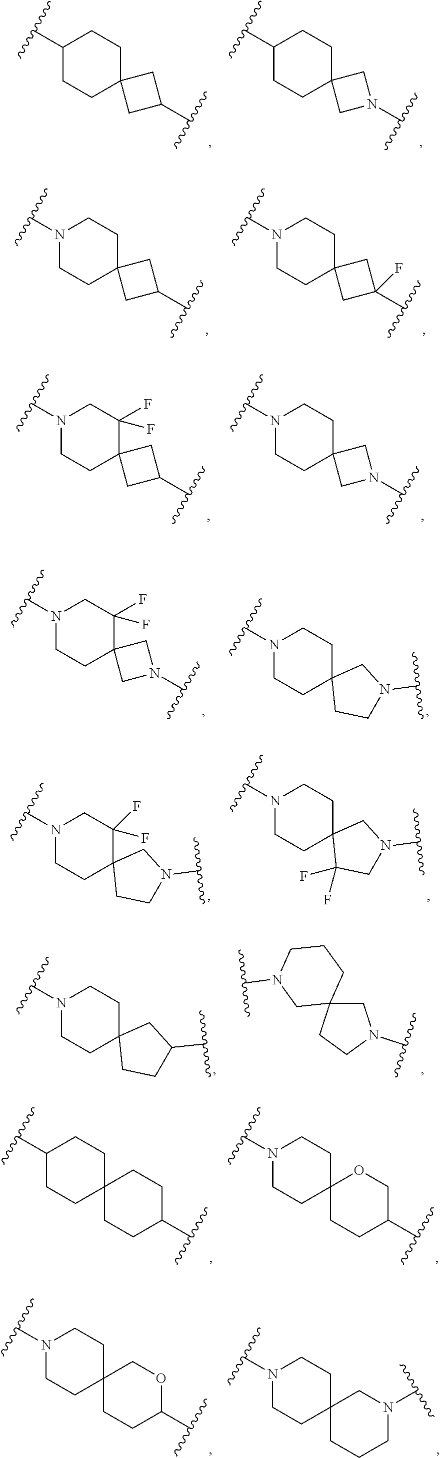

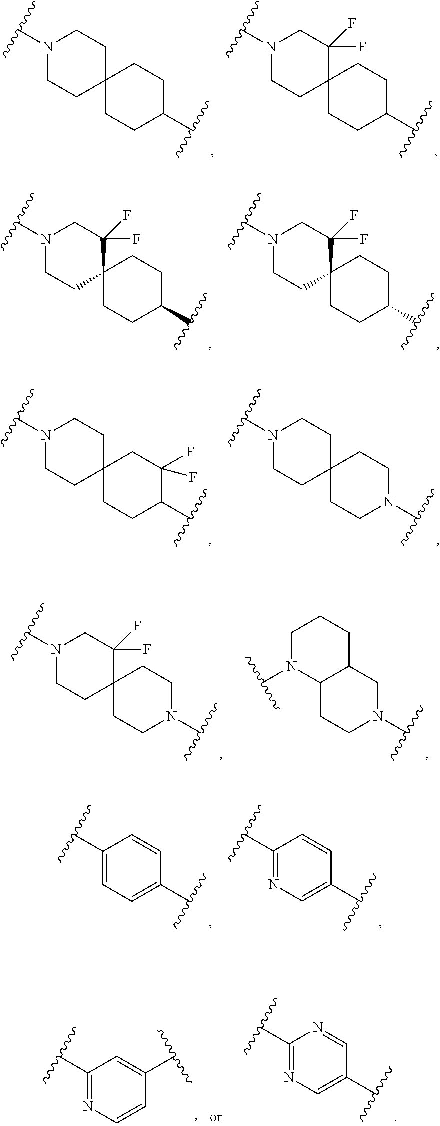

In some embodiments, -Cy- is as depicted in the compounds of Table 1, below.

In some embodiments, L is a covalent bond, —CH₂—, —CH₂CH₂—, —C(O)—,

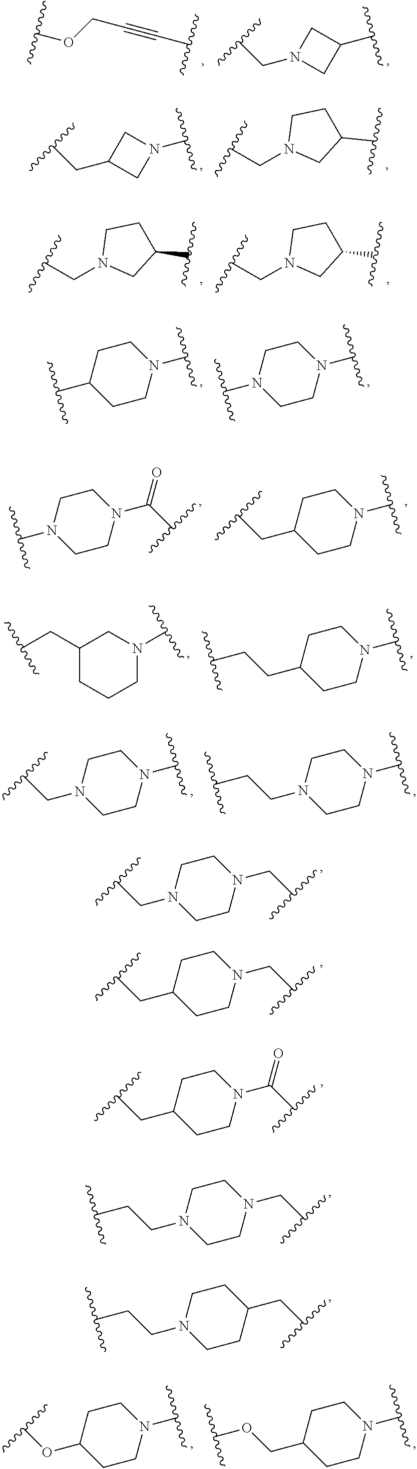

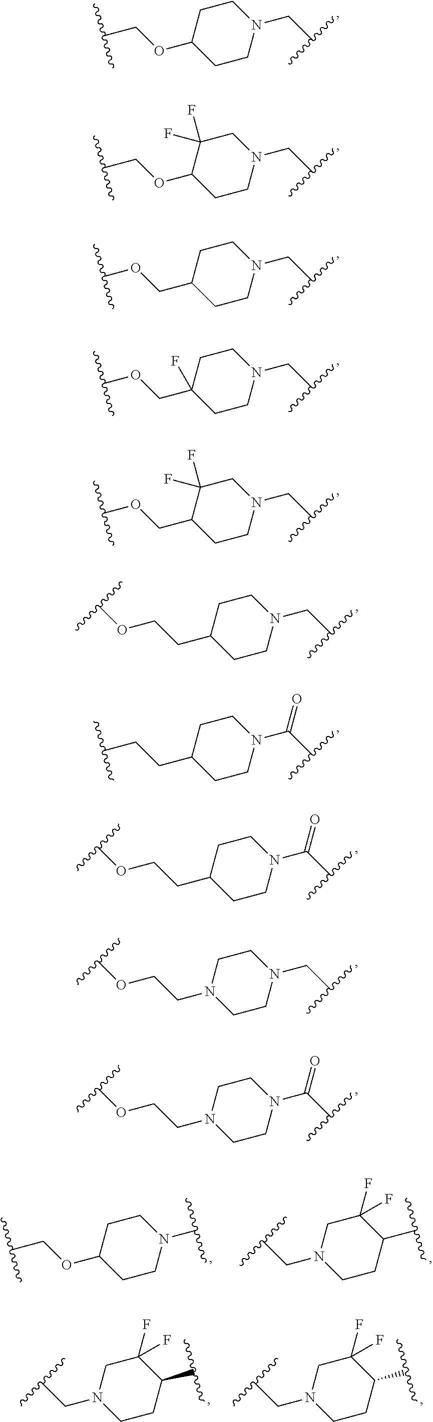

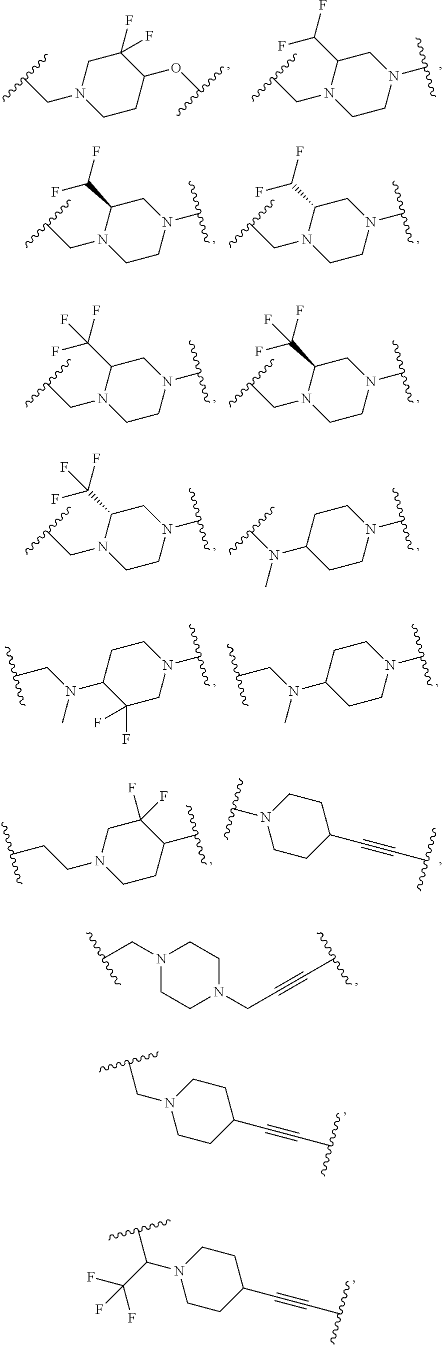

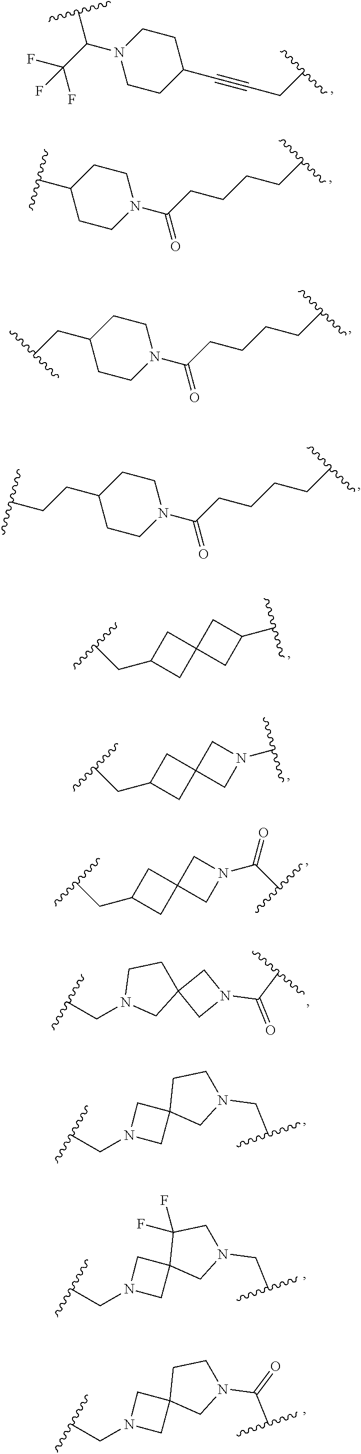

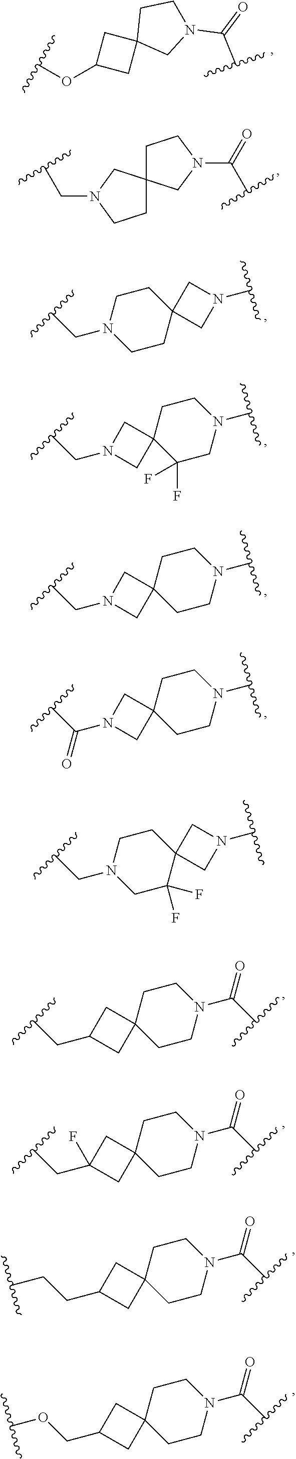

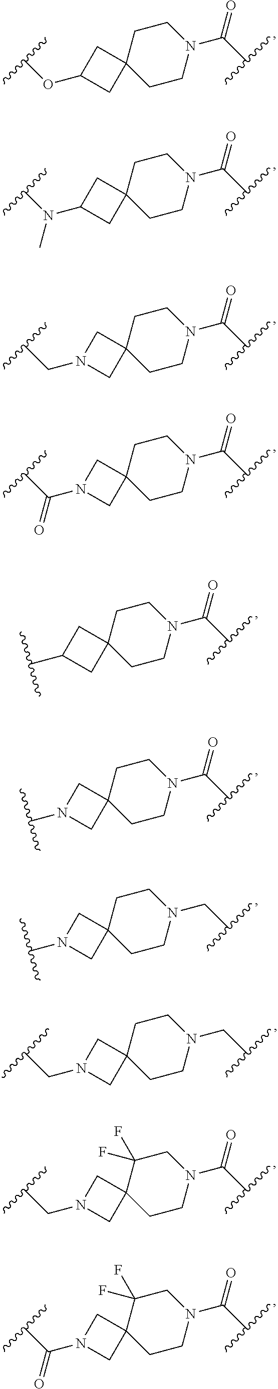

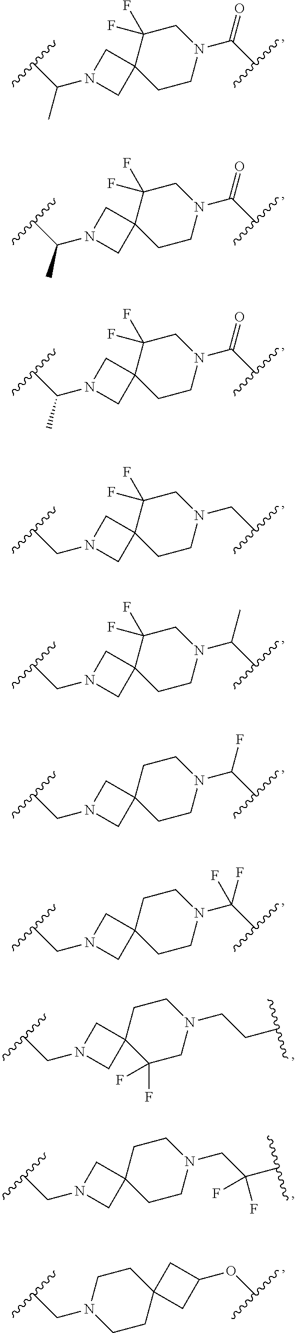

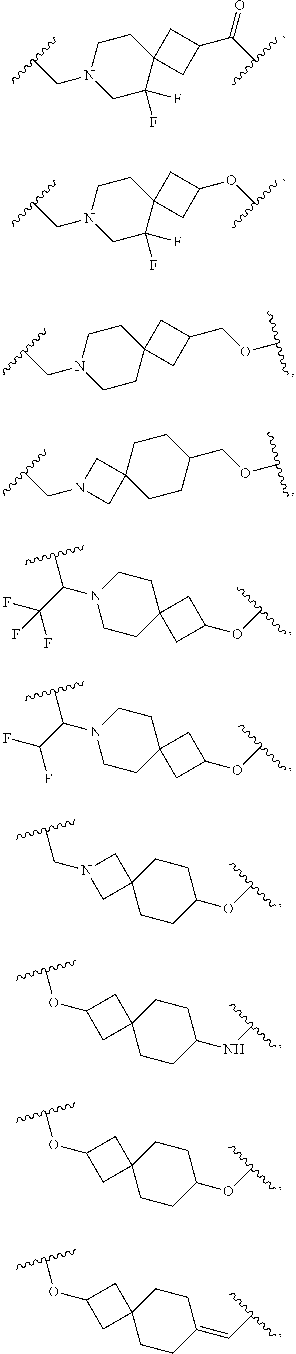

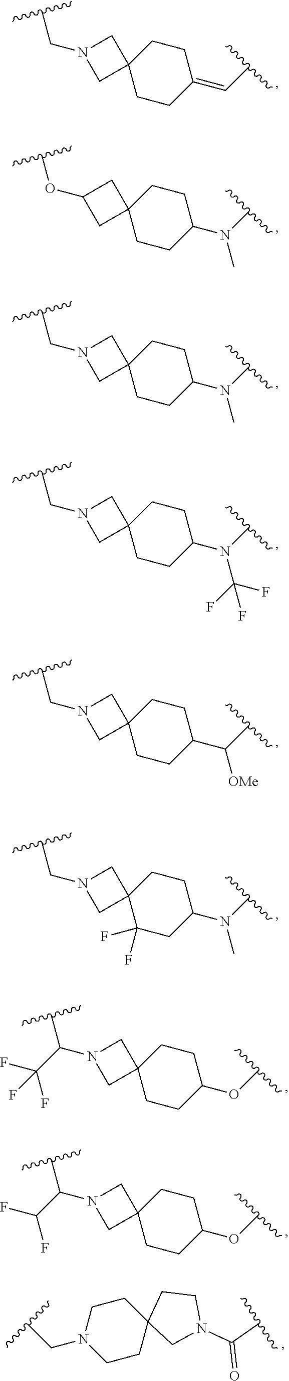

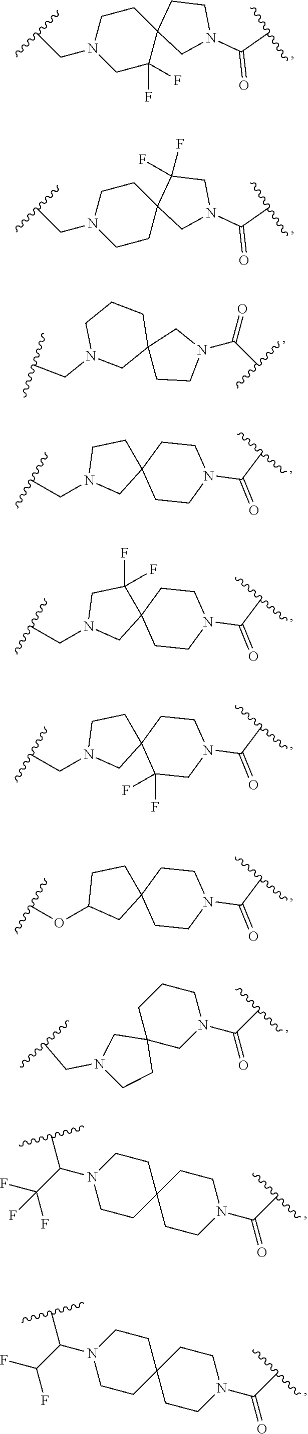

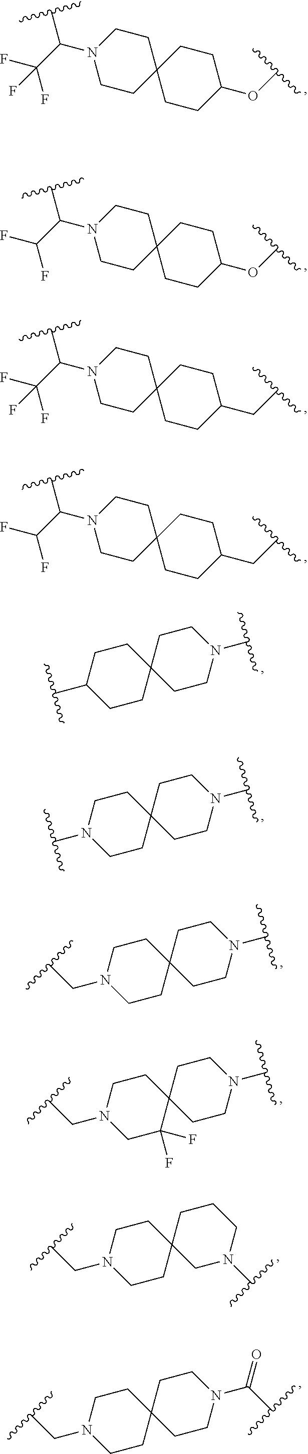

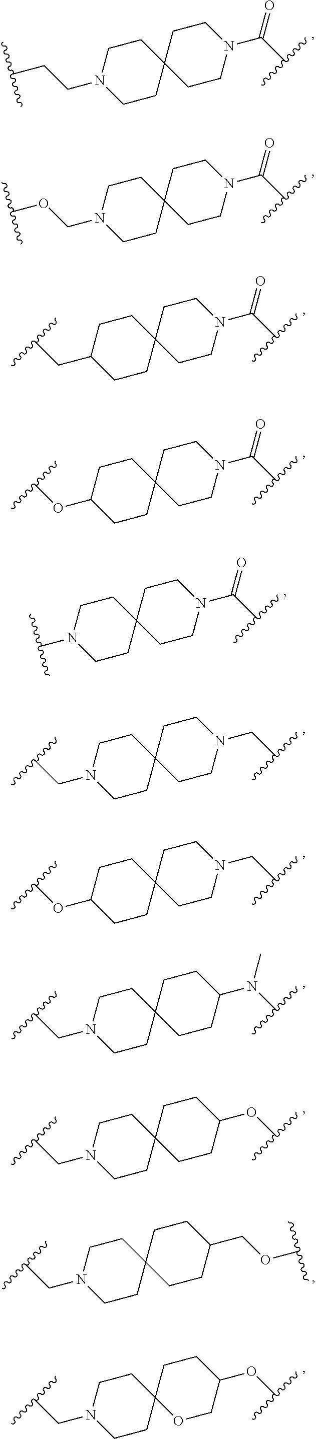

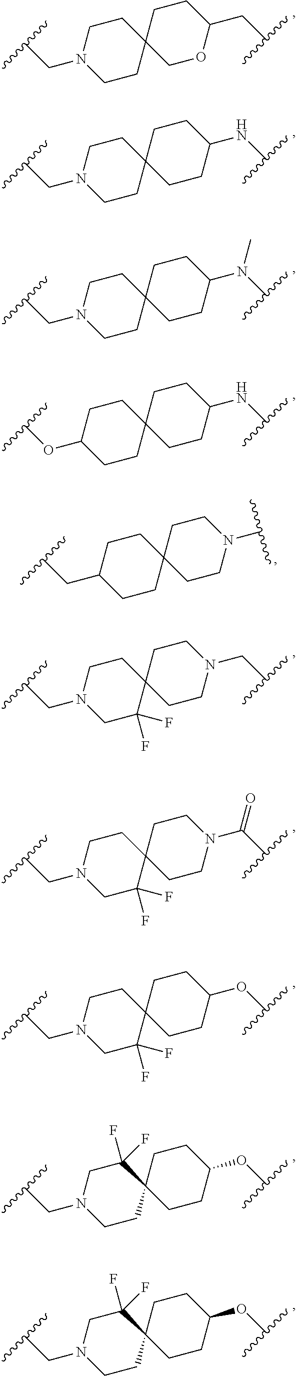

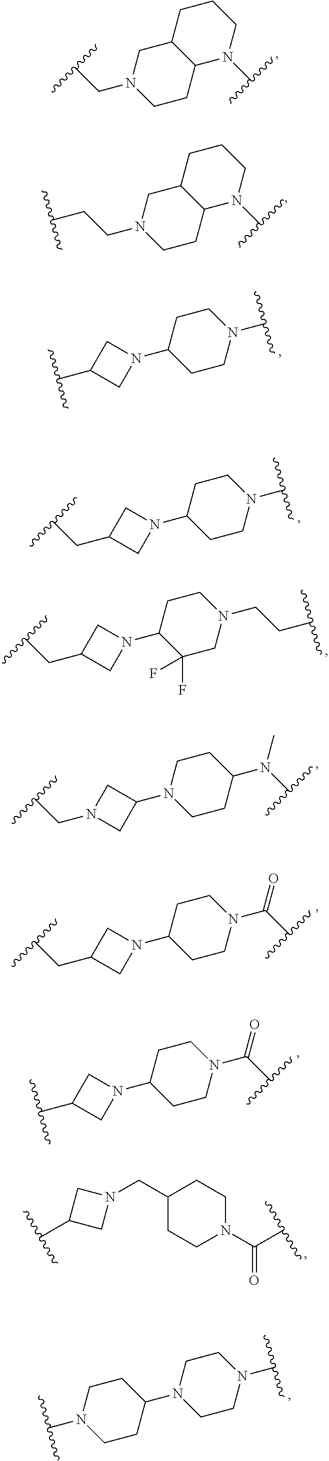

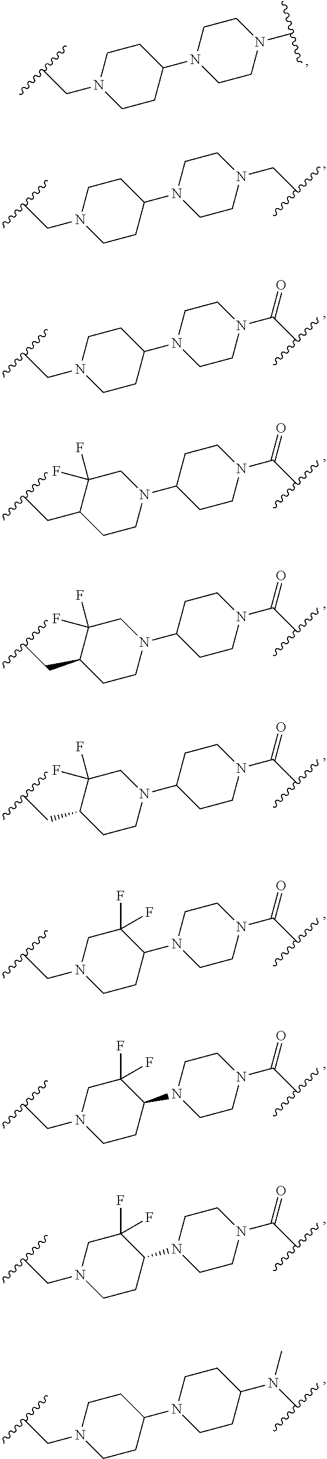

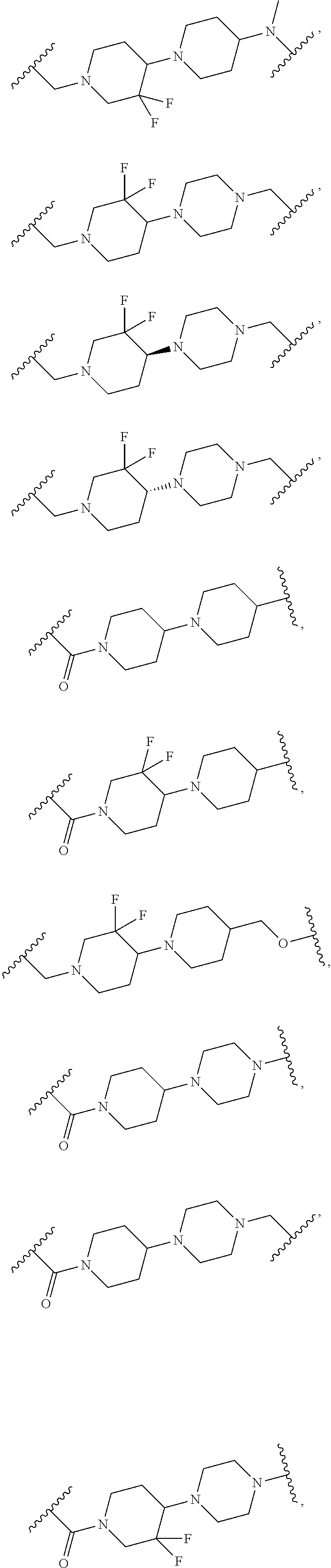

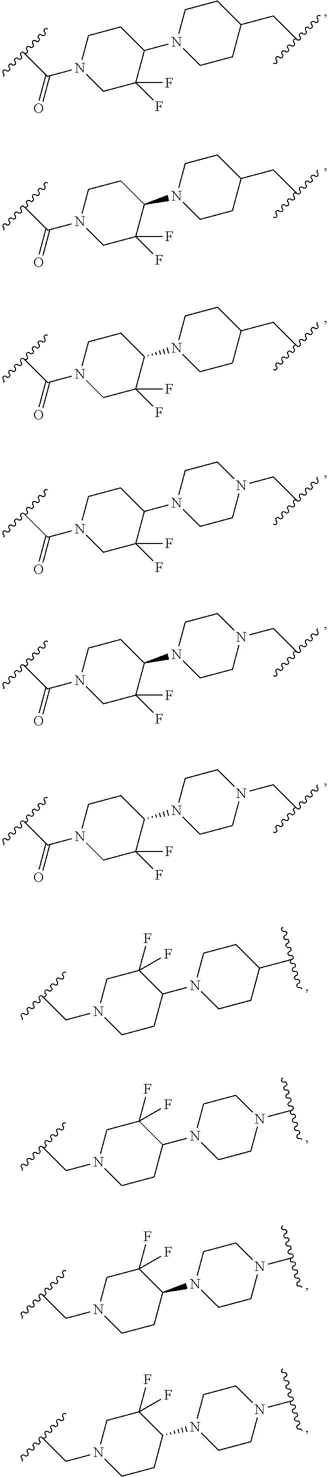

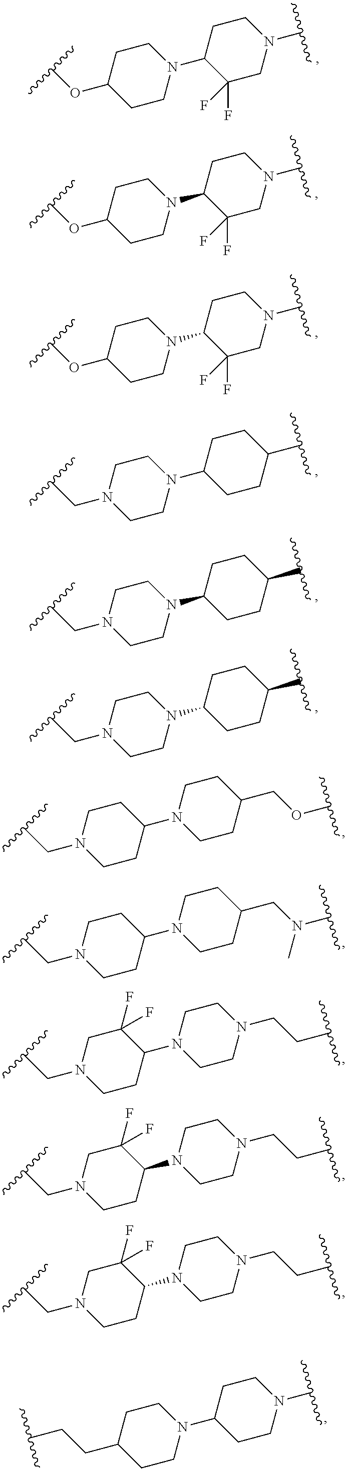

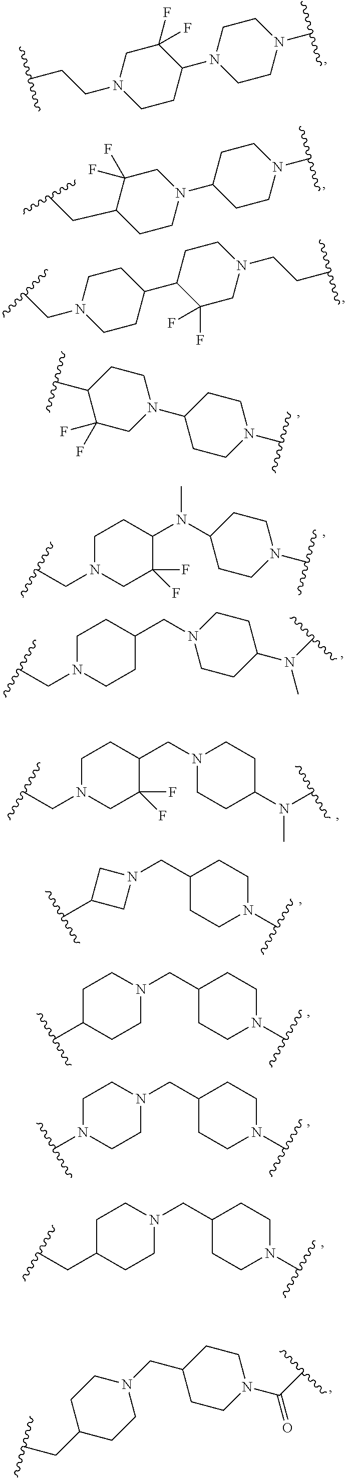

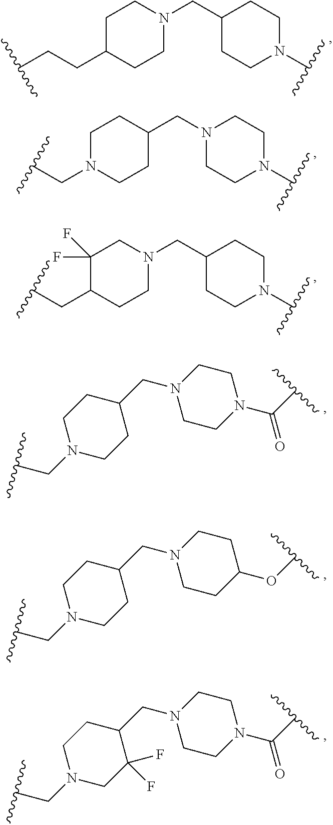 [SMILES: *CCC1CCN(CC2CCN(*)CC2)CC1 ; *CN1CCC(CN2CCN(*)CC2)CC1 ; *CC1CCN(CC2CCN(*)CC2)CC1(F)F ; *CN1CCC(CN2CCN(C(*)=O)CC2)CC1 ; *CN1CCC(CN2CCC(O*)CC2)CC1 ; *CN1CCC(CN2CCN(C(*)=O)CC2)C(F)(F)C1]

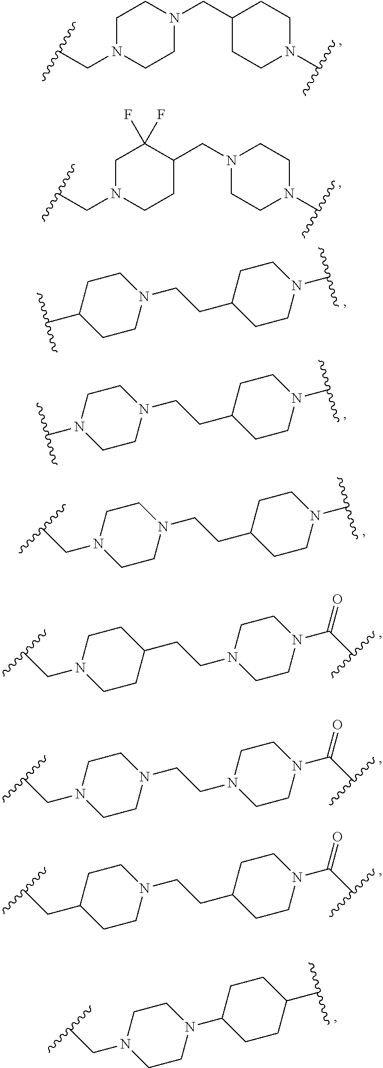

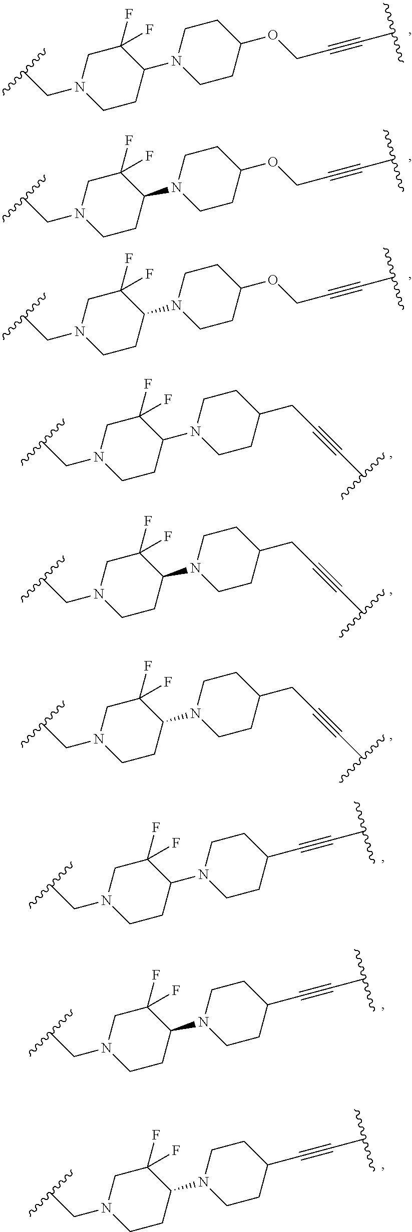

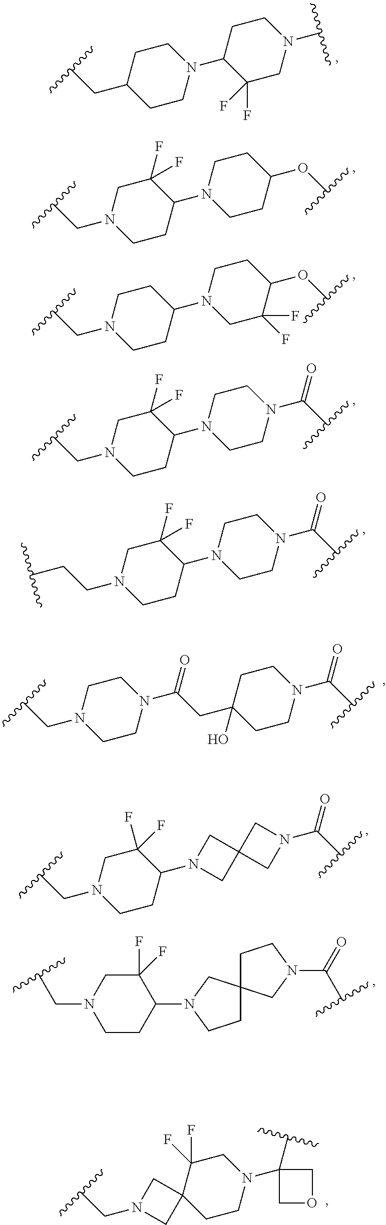

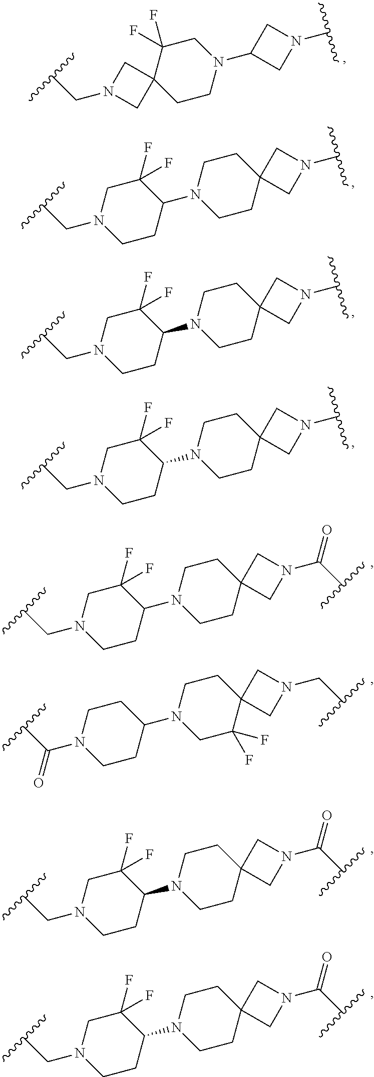

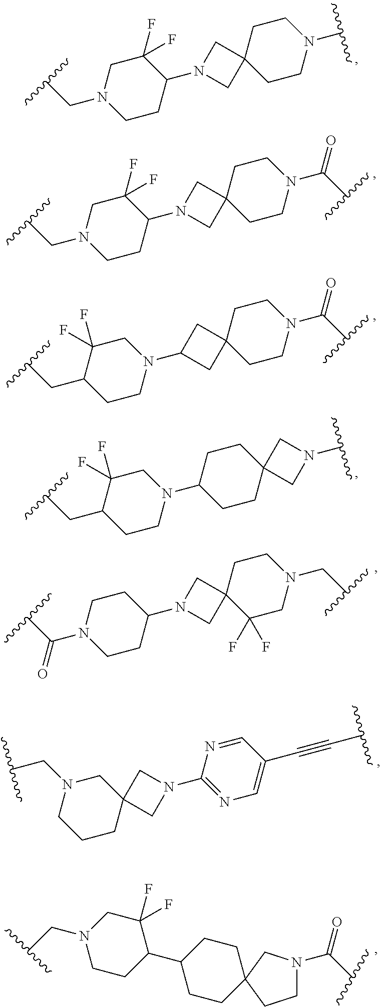

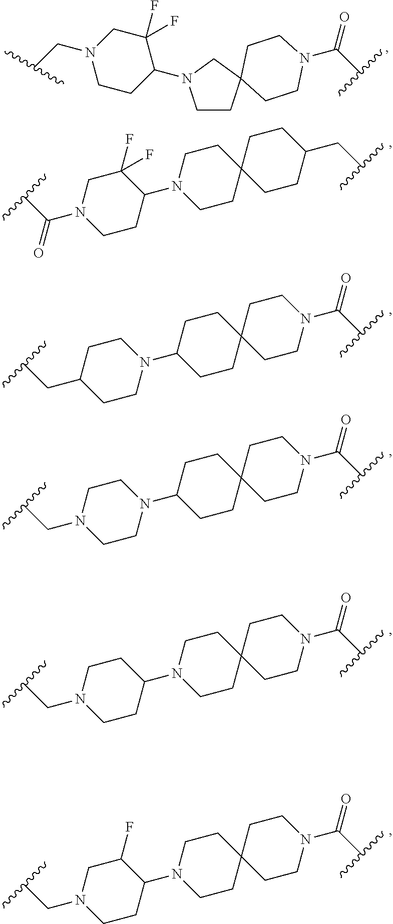

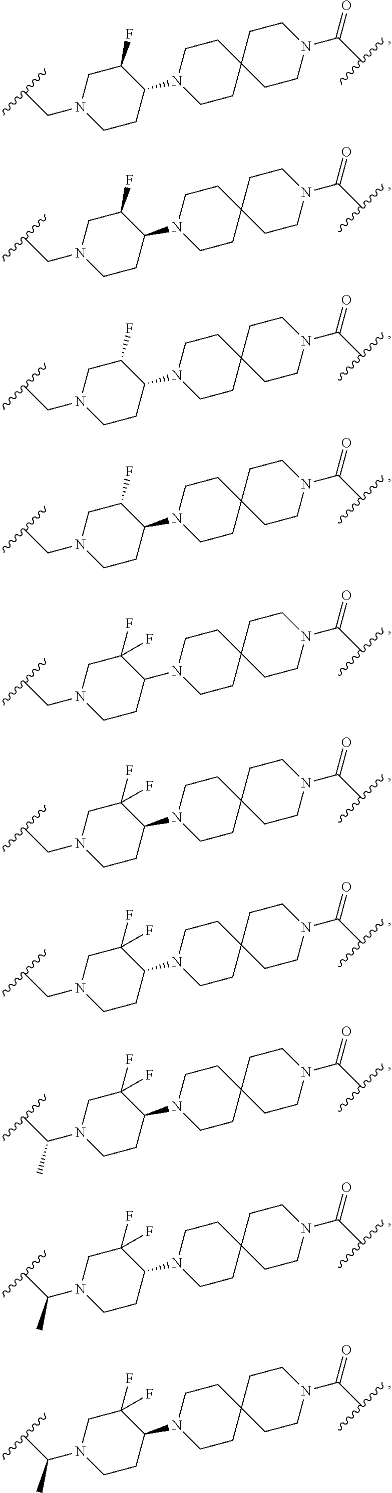

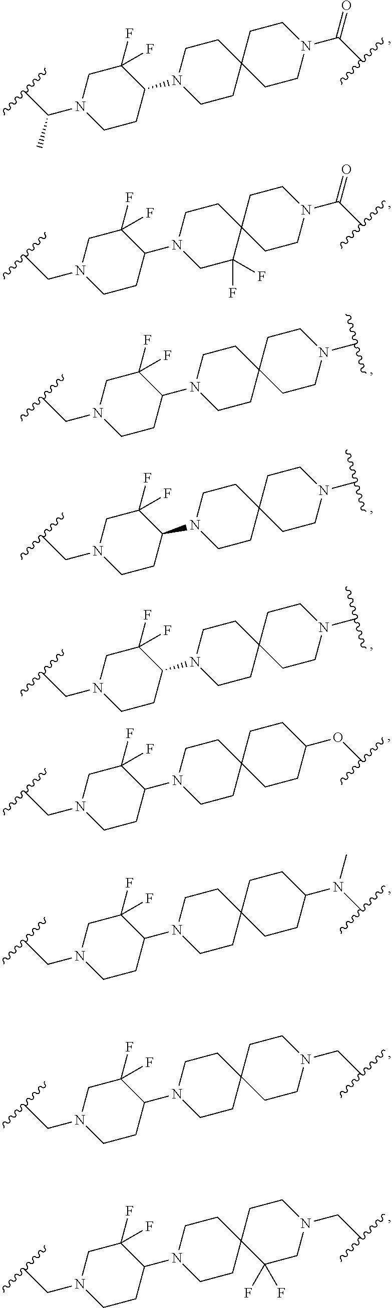

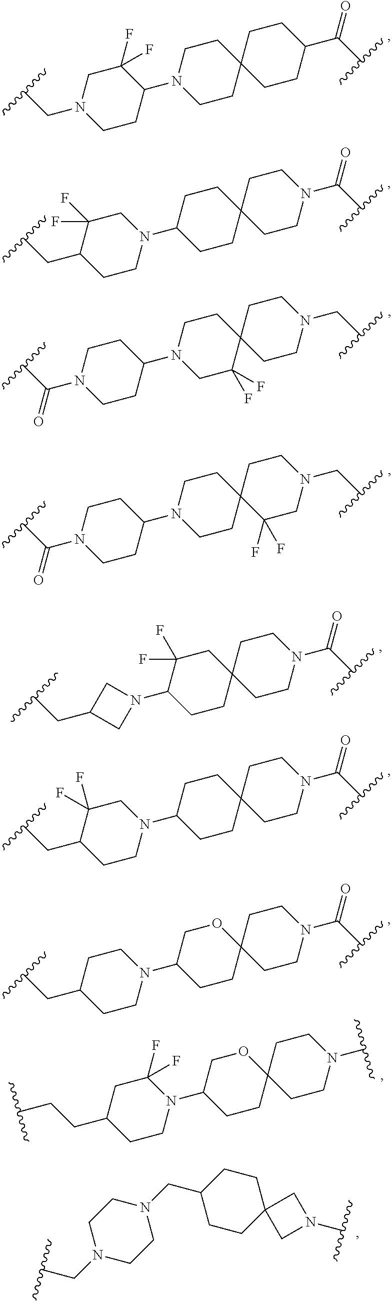

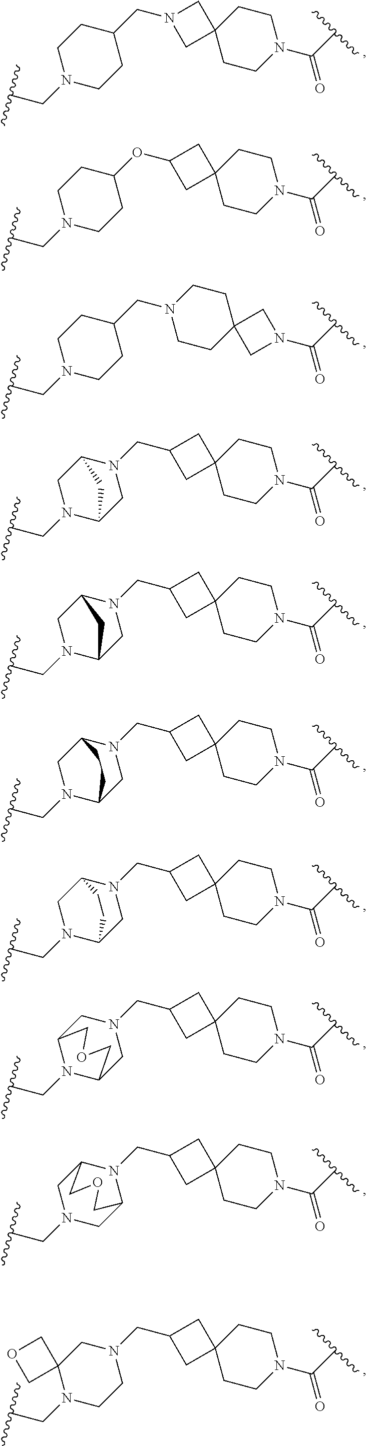

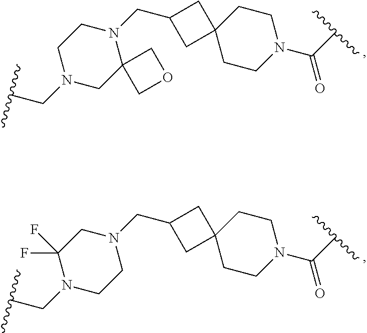 [SMILES: *CN1CCN(CC2CC3(CCN(C(*)=O)CC3)C2)C2(COC2)C1 ; *CN1CCN(CC2CC3(CCN(C(*)=O)CC3)C2)CC1(F)F]

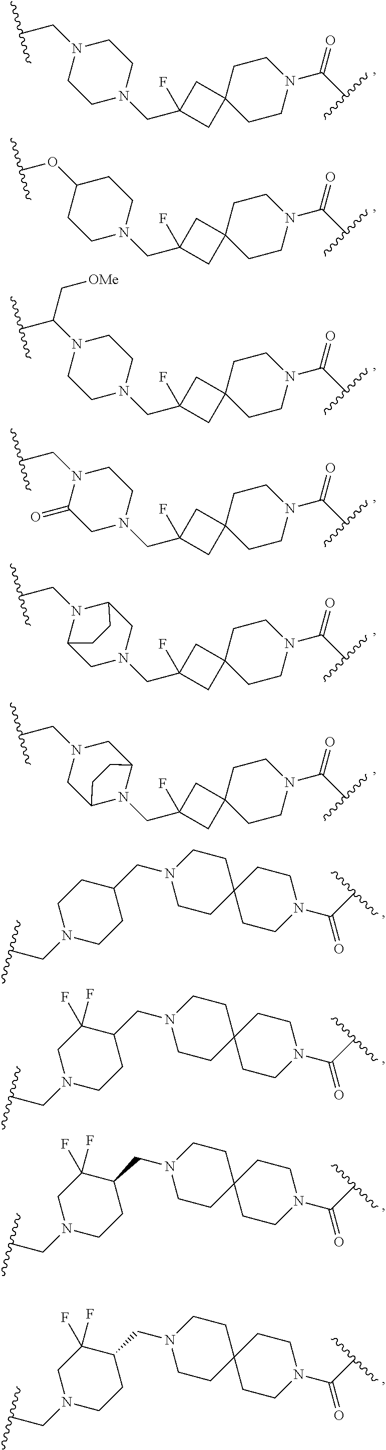

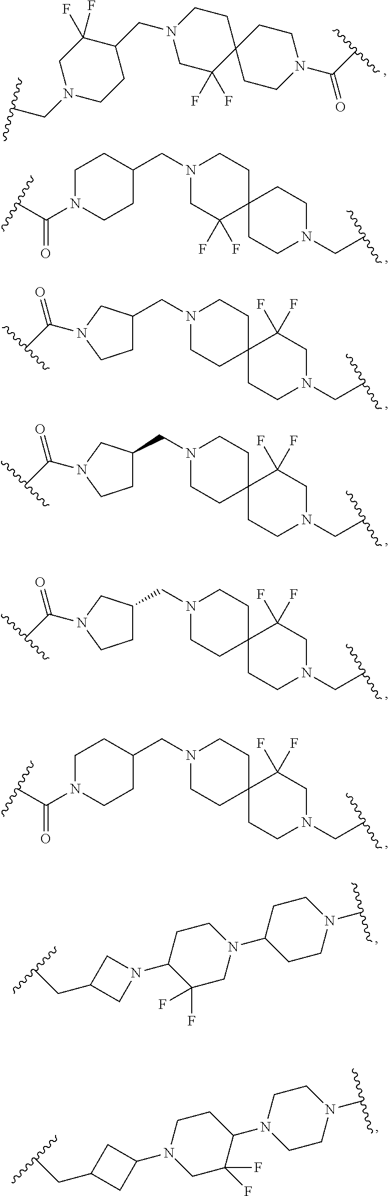

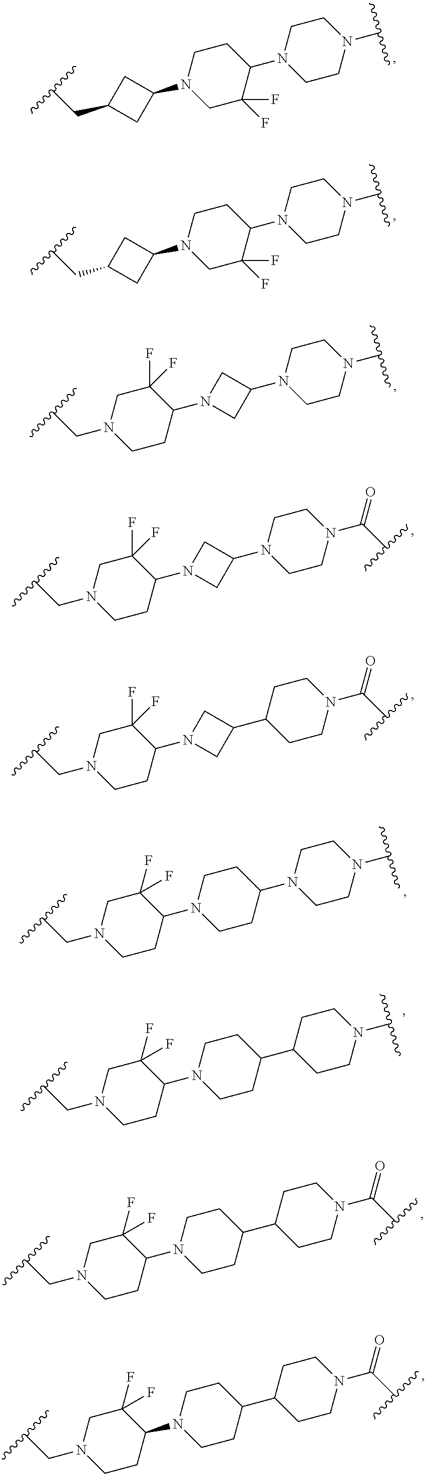

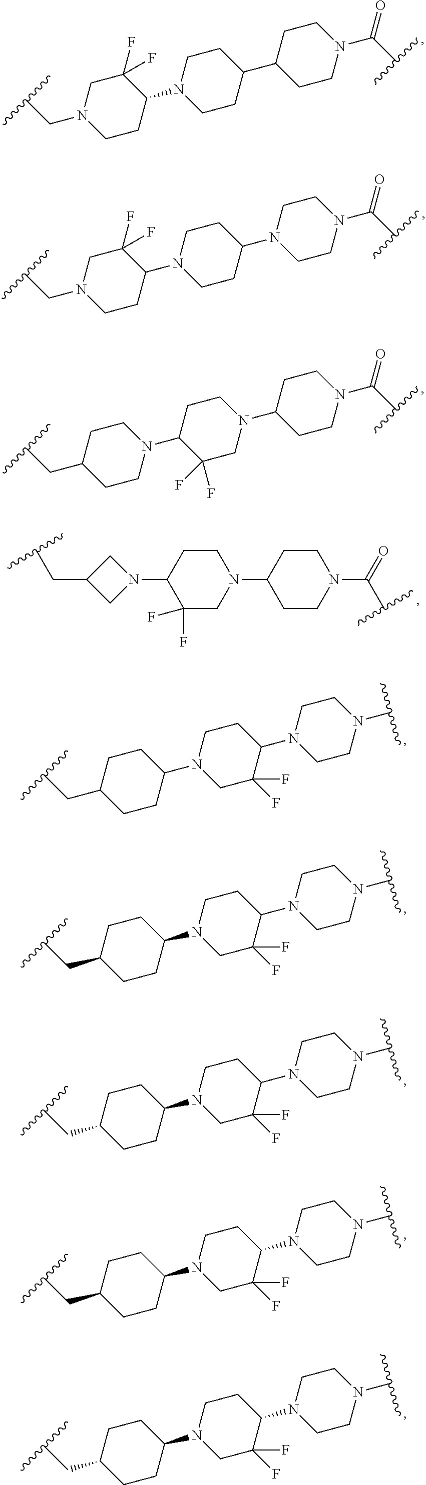

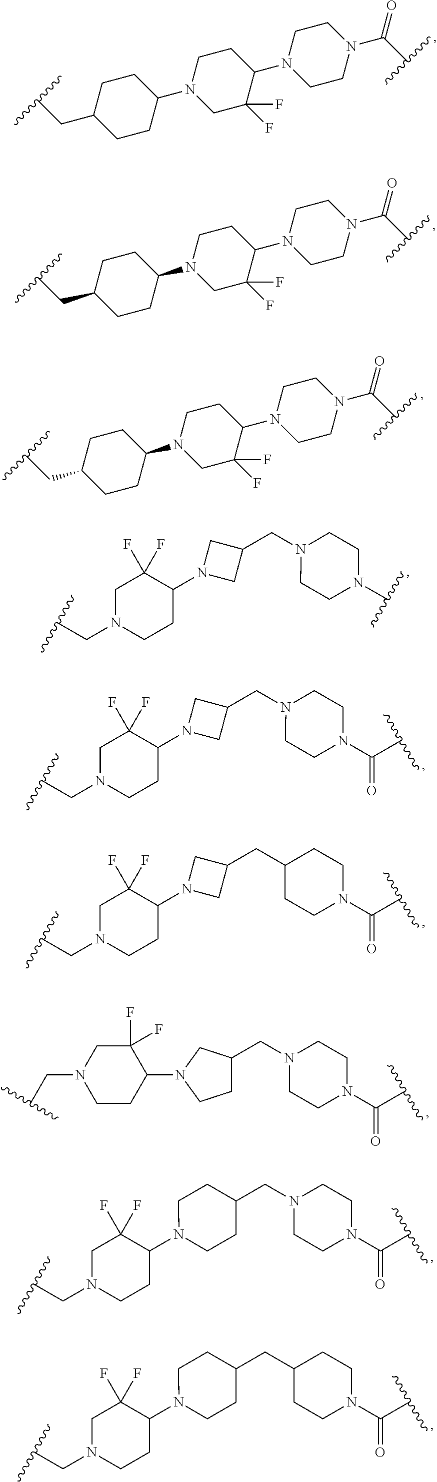

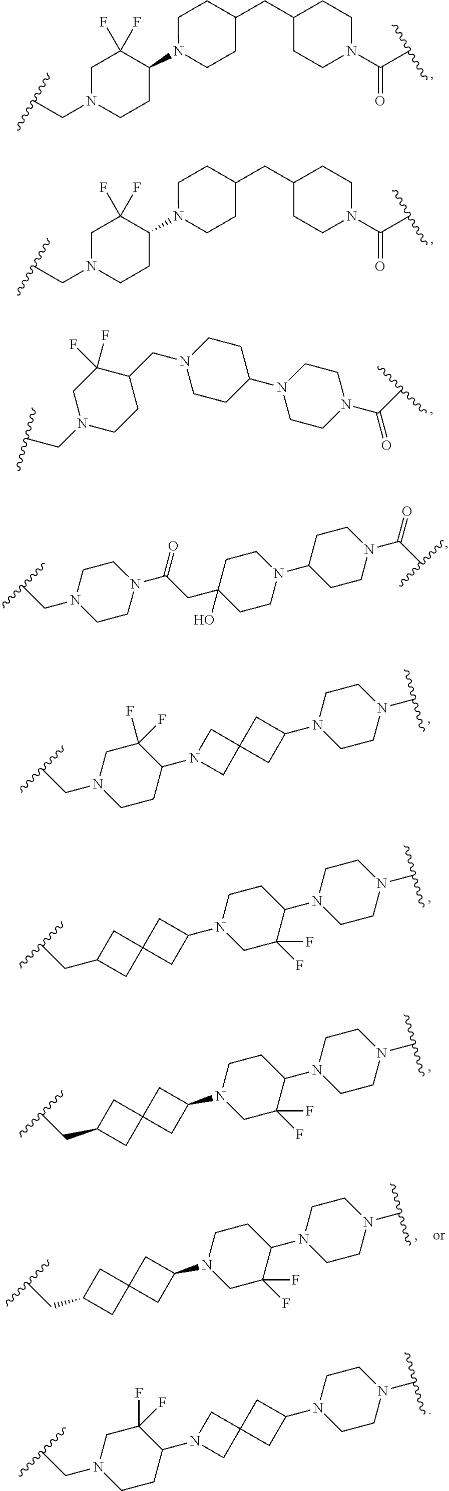

In some embodiments, L is as depicted in the compounds of Table 1, below.

As defined herein and described above, each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 3-7 membered saturated or partially unsaturated carbocyclic. In some embodiments, R is an optionally substituted 3-7 membered saturated or partially unsaturated heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur.

In some embodiments, R is as depicted in the compounds of Table 1, below.

As defined herein and described above, each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{A} is an optionally substituted C₁₋₆ aliphatic. In some embodiments, R^{A} is an optionally substituted phenyl. In some embodiments, R^{A} is an optionally substituted 3-7 membered saturated or partially unsaturated carbocyclic. In some embodiments, R^{A} is an optionally substituted 3-7 membered saturated or partially unsaturated heterocyclic ring having 1-2 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur.

In some embodiments, R^{A} is C₁₋₆ alkyl. In some embodiments, R^{A} is C₁₋₆haloalkyl.

In some embodiments, R^{A} is methyl, ethyl, isopropyl, cyclopropyl, —CHF₂, or CF₃.

In some embodiments, R^{A} is as depicted in the compounds of Table 1, below.

Without limitation, the point of attachment of bivalent groups of this disclosure (e.g., Y¹, Ring A, L, X¹, X², X³, and Ring A, Ring C, Ring D, Ring E, and Ring F) are not directional, e.g., when L is

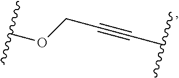 [SMILES: *C#CCO*]

it includes attachment to Ring A and X¹ as

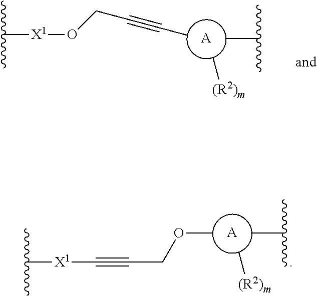

In some embodiments, the present invention provides the compound of formula I-a or I-a′ as a compound of formula I-a-1:

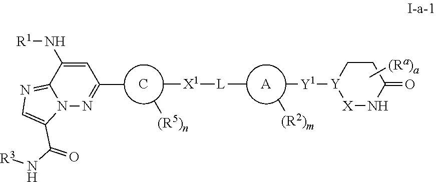

or a pharmaceutically acceptable salt thereof, wherein each of the variables is as defined in formula I-a or I-a′ above and described in embodiments herein, both singly and in combination.

In some embodiments, the present invention provides the compound of formula I-a or I-a′ as a compound of formula I-a-2:

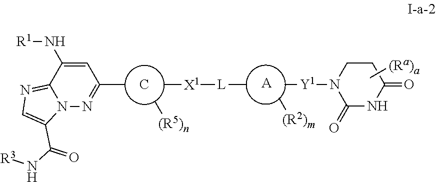

or a pharmaceutically acceptable salt thereof, wherein each of the variables is as defined in formula I-a or I-a′ above and described in embodiments herein, both singly and in combination.

In some embodiments, the present invention provides the compound of formula I-a or I-a′ as a compound of formula I-a-3:

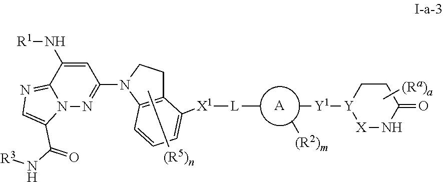

or a pharmaceutically acceptable salt thereof, wherein each of the variables is as defined in formula I-a or I-a′ above and described in embodiments herein, both singly and in combination.

In some embodiments, the present invention provides the compound of formula I-a or I-a′ as a compound of formula I-a-4:

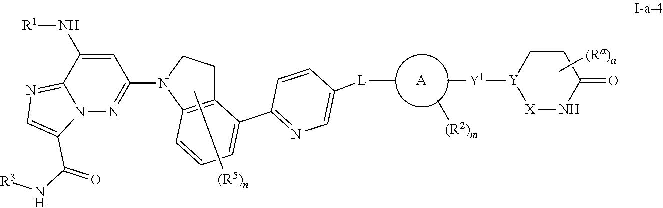

or a pharmaceutically acceptable salt thereof, wherein each of the variables is as defined in formula I-a or I-a′ above and described in embodiments herein, both singly and in combination.

In some embodiments, the present invention provides the compound of formula I-a or I-a′ as a compound of formula I-a-5:

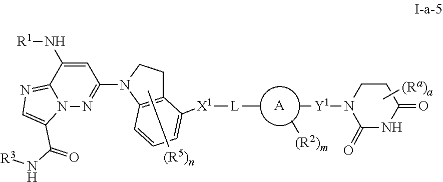

or a pharmaceutically acceptable salt thereof, wherein each of the variables is as defined in formula I-a or I-a′ above and described in embodiments herein, both singly and in combination.

In some embodiments, the present invention provides the compound of formula I-a or I-a′ as a compound of formula I-a-6:

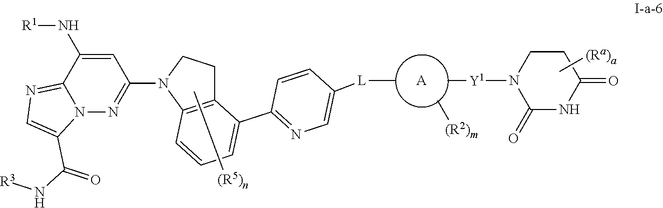

or a pharmaceutically acceptable salt thereof, wherein each of the variables is as defined in formula I-a or I-a′ above and described in embodiments herein, both singly and in combination.

In some embodiments, the present invention provides the compound of formula I-a as a compound of formula I-a-7:

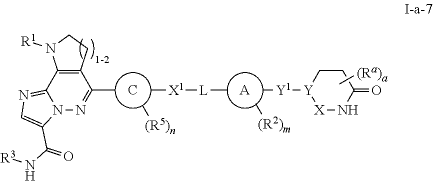

or a pharmaceutically acceptable salt thereof, wherein each of the variables is as defined in formula I-a or I-a′ above and described in embodiments herein, both singly and in combination.

In some embodiments, the present invention provides the compound of formula I-a or I-a′ as a compound of formula I-a-8:

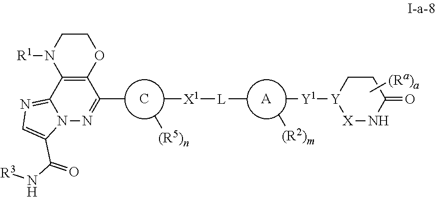

or a pharmaceutically acceptable salt thereof, wherein each of the variables is as defined in formula I-a or I-a′ above and described in embodiments herein, both singly and in combination.

In some embodiments, the present invention provides the compound of formula I-a or I-a′ as a compound of formula I-a-9:

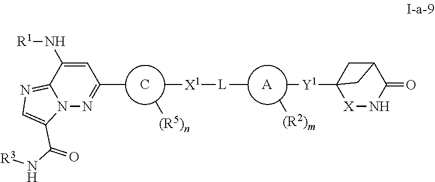

or a pharmaceutically acceptable salt thereof, wherein each of the variables is as defined in formula I-a or I-a′ above and described in embodiments herein, both singly and in combination.

In some embodiments, the present invention provides the compound of formula I-a or I-a′ as a compound of any one of the following formulae:

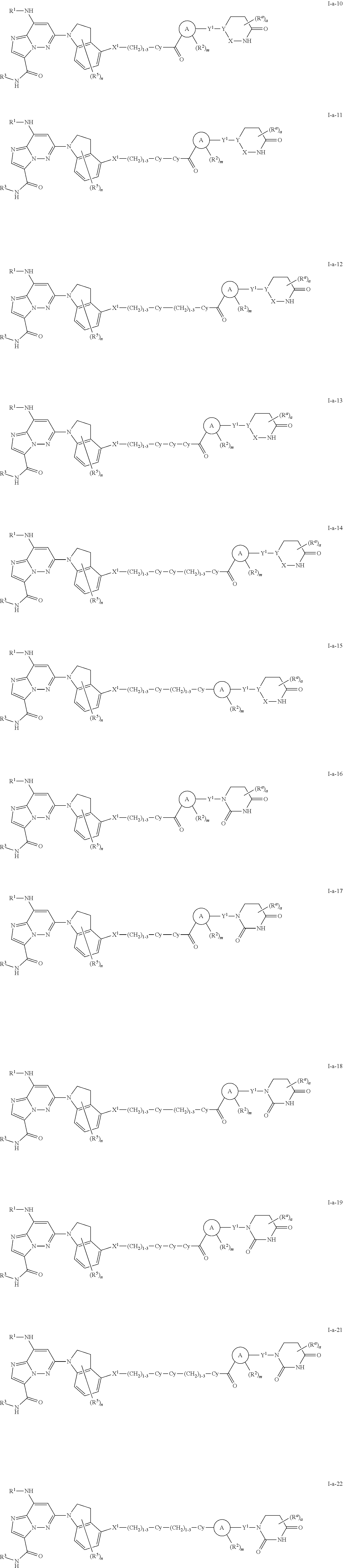

or a pharmaceutically acceptable salt thereof, wherein each of the variables is as defined in formula I-a or I-a′ above and described in embodiments herein, both singly and in combination.

In some embodiments, the present invention provides a compound of formula I-a-23:

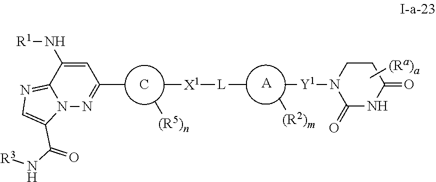

or a pharmaceutically acceptable salt thereof, wherein:
X¹ is a covalent bond,

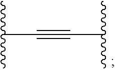 [SMILES: *C#C*]

or an optionally substituted ring selected from 4-6 membered saturated carbocyclylenyl or heterocyclylenyl having 1-2 heteroatoms independently selected from nitrogen, oxygen, and sulfur, phenylenyl, 5-6 membered heteroarylenyl having 1-2 heteroatoms independently selected from nitrogen, oxygen, and sulfur, and a 5-8 membered saturated bridged bicyclic heterocyclylenyl having 1-2 heteroatoms independently selected from nitrogen, oxygen, and sulfur;
Y¹ is a covalent bond, —O—, —S—, —NR—, or —C(O)NR—;
Ring A is a ring selected from phenylenyl, pyridinylenyl,

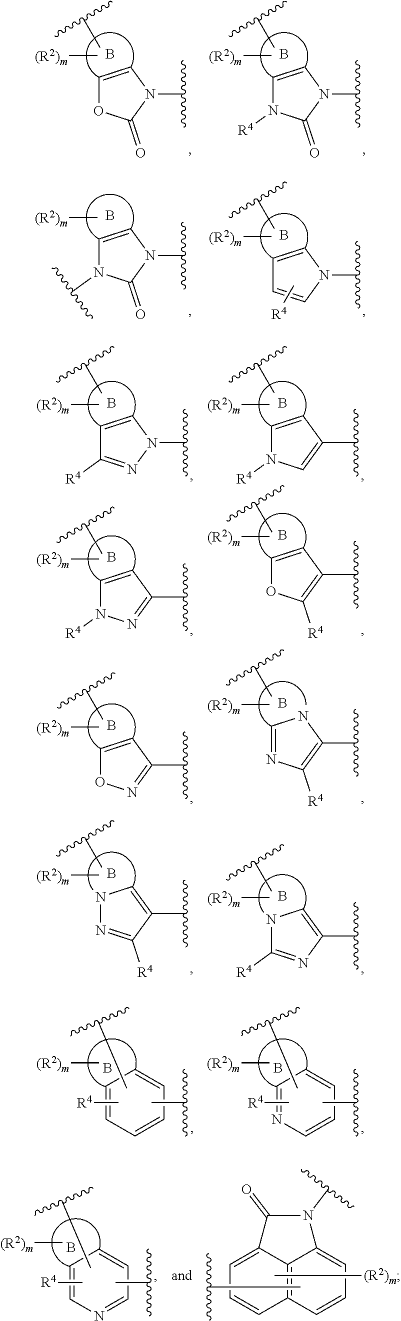

Ring B is a fused ring selected from benzo, a 5-6 membered saturated or partially unsaturated heterocyclyl having 1-3 heteroatoms independently selected from nitrogen, oxygen, and sulfur, or a 5-6 membered heteroaryl containing 1-4 heteroatoms independently selected from nitrogen, oxygen, and sulfur;
Ring C is phenylenyl, a 9-10 membered saturated or partially unsaturated bicyclic heterocyclylenyl having 1-3 heteroatoms independently selected from nitrogen, oxygen, and sulfur, 8-10 membered bicyclic heteroarylenyl having 1-3 heteroatoms independently selected from nitrogen, oxygen, and sulfur, 12-15 membered saturated or partially unsaturated tricyclic spirocyclic heterocyclylenyl having 1-3 heteroatoms independently selected from nitrogen, oxygen;
R^{a} is hydrogen, halogen, —CN, —OR, oxo, C₁₋₆alkyl, C₁₋₆ haloalkyl, C₃₋₆ cycloalkyl, or:
   two R^{a} on the same carbon atom or adjacent carbon atoms connect to form a 3-6 membered saturated carbocyclic or heterocyclic ring having 1-2 heteroatoms selected from nitrogen, oxygen, and sulfur;
a is 0, 1, or 2;
R^{b} is hydrogen, or:
   R^{b} connects with the nitrogen where R¹ is attached to form an optionally substituted 5-6 membered partially unsaturated or aromatic heterocyclic ring having 0-2 heteroatoms in addition to the nitrogen atom where R¹ is attached selected from nitrogen, oxygen, and sulfur;
R¹ is hydrogen, C₁₋₆ alkyl, C₁₋₆ haloalkyl, or C₃₋₆ cycloalkyl;
each R² and R⁵ is independently hydrogen, halogen, —CN, —NO₂, —OR, oxo, —SR, —NR₂, —SiR₃, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, or R^{A};
m and n are independently 0, 1, 2, 3, or 4;
R³ is hydrogen, or a C₁₋₆ alkyl or C₄₋₆ cycloalkyl optionally substituted with 1-2 substituents selected from halogen, —CN, C₁₋₆alkyl, C₁₋₆haloalkyl, and —OR, or:
   R³ and R⁵ are joined by a bivalent, saturated or partially unsaturated, straight or branched C₃₋₆ hydrocarbon chain, wherein 0-2 methylene units of the chain are independently replaced by —CR₂—, —CRF—, —CF₂—, —O—, —NR—, —C(O)—;
R⁴ is hydrogen, halogen, C₁₋₆ alkyl, C₁₋₆ haloalkyl, C₃₋₆ cycloalkyl, —OC₁₋₆ alkyl, or —OC₁₋₆ haloalkyl; L is a covalent bond, —CR₂—, —CRF—, —CF₂—, —O—, —NR—, —C(O)—, or a bivalent, saturated or partially unsaturated, straight or branched C₁₋₁₀ hydrocarbon chain, wherein 0-6 methylene units of L are independently replaced by -Cy-, —CR₂—, —CRF—, —CF₂—, —CR(OR)—, —O—, —NR—, —C(O)—;
each -Cy- is independently an optionally substituted bivalent ring selected from phenylenyl, 8-10 membered bicyclic arylenyl, 4-7 membered saturated or partially unsaturated carbocyclylenyl, 6-11 membered saturated or partially unsaturated spirocyclic or bridged bicyclic carbocyclylenyl, 8-10 membered bicyclic saturated or partially unsaturated carbocyclylenyl, 4-7 membered saturated or partially unsaturated heterocyclylenyl having 1-2 heteroatoms independently selected from nitrogen, oxygen, and sulfur, 6-11 membered saturated or partially unsaturated spirocyclic or bridged bicyclic heterocyclylenyl having 1-2 heteroatoms independently selected from nitrogen, oxygen, and sulfur, 8-10 membered bicyclic saturated or partially unsaturated heterocyclylenyl having 1-2 heteroatoms independently selected from nitrogen, oxygen, and sulfur, 5-6 membered heteroarylenyl having 1-4 heteroatoms independently selected from nitrogen, oxygen, and sulfur, and 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur; and
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, the present invention provides the compound of formula I-a-23 as a compound of formula I-a-24:

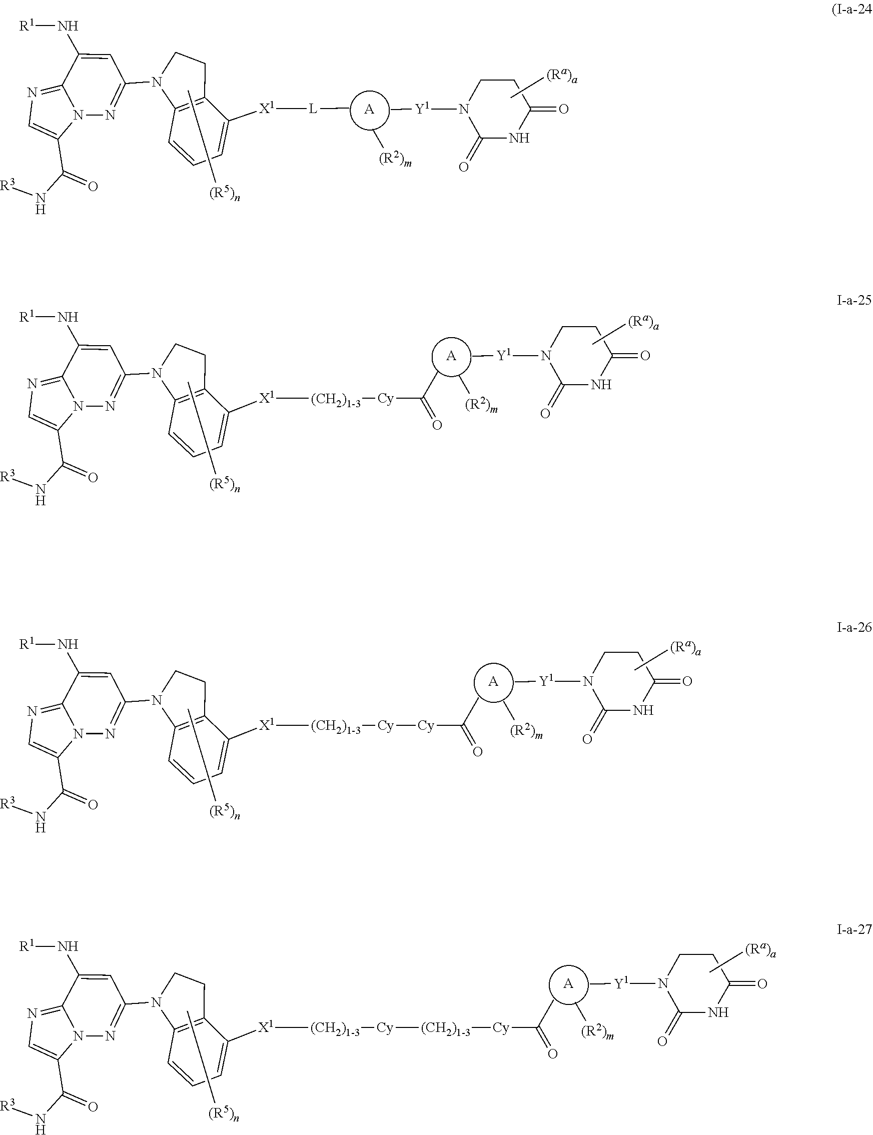

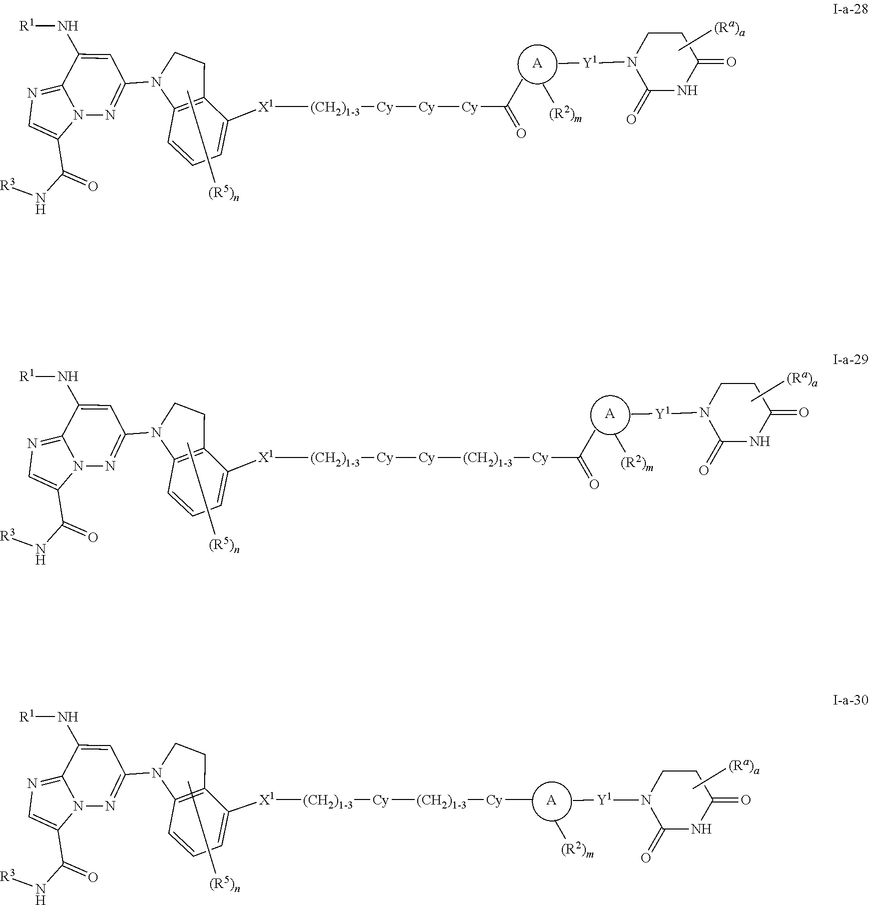

or a pharmaceutically acceptable salt thereof, wherein each of the variables is as defined in formula I-a-23 above and described in embodiments herein, both singly and in combination.

In some embodiments, the present invention provides the compound of formula I-b as a compound of formula I-b-1:

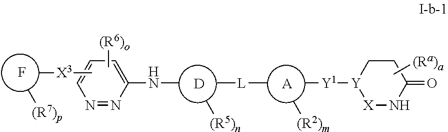

or a pharmaceutically acceptable salt thereof, wherein each of the variables is as defined in formula I-b above and described in embodiments herein, both singly and in combination.

In some embodiments, the present invention provides the compound of formula I-b as a compound of formula I-b-2:

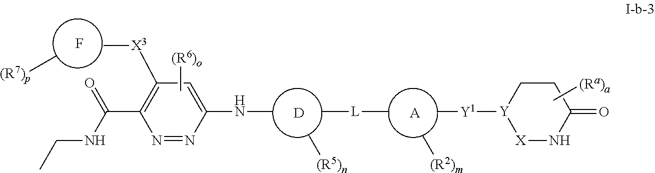

or a pharmaceutically acceptable salt thereof, wherein each of the variables is as defined in formula I-b above and described in embodiments herein, both singly and in combination.

In some embodiments, the present invention provides the compound of formula I-b as a compound of formula I-b-3:

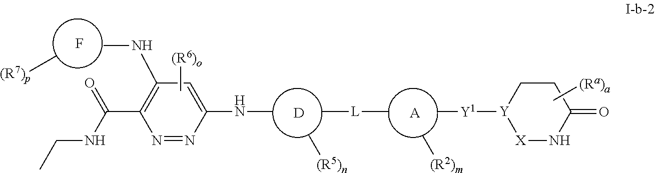

or a pharmaceutically acceptable salt thereof, wherein each of the variables is as defined in formula I-b above and described in embodiments herein, both singly and in combination.

In some embodiments, the present invention provides the compound of formula I-b as a compound of formula I-b-4:

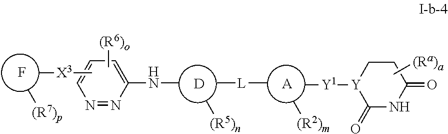

or a pharmaceutically acceptable salt thereof, wherein each of the variables is as defined in formula I-b above and described in embodiments herein, both singly and in combination.

In some embodiments, the present invention provides the compound of formula I-b as a compound of formula I-b-5:

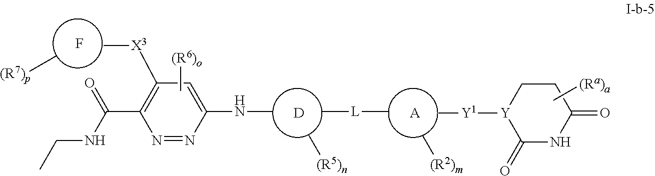

or a pharmaceutically acceptable salt thereof, wherein each of the variables is as defined in formula I-b above and described in embodiments herein, both singly and in combination.

In some embodiments, the present invention provides the compound of formula I-b as a compound of formula I-b-6:

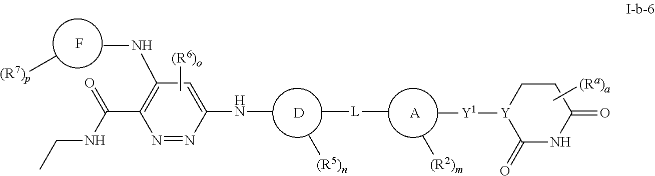

or a pharmaceutically acceptable salt thereof, wherein each of the variables is as defined in formula I-b above and described in embodiments herein, both singly and in combination.

In some embodiments, the present invention provides the compound of formula I-b as a compound of formula I-b-7:

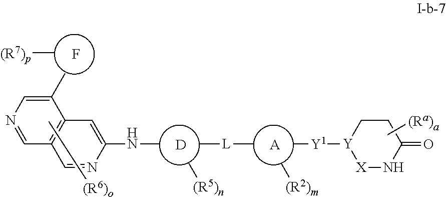

or a pharmaceutically acceptable salt thereof, wherein each of the variables is as defined in formula I-b above and described in embodiments herein, both singly and in combination.

In some embodiments, the present invention provides the compound of formula I-b as a compound of formula I-b-8:

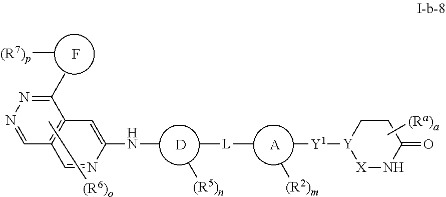

or a pharmaceutically acceptable salt thereof, wherein each of the variables is as defined in formula I-b above and described in embodiments herein, both singly and in combination

In some embodiments, the present invention provides the compound of formula I-c as a compound of formula I-c-1:

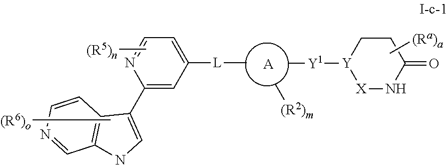

or a pharmaceutically acceptable salt thereof, wherein each of the variables is as defined in formula I-c above and described in embodiments herein, both singly and in combination.

In some embodiments, the present invention provides the compound of formula I-c as a compound of formula I-c-2:

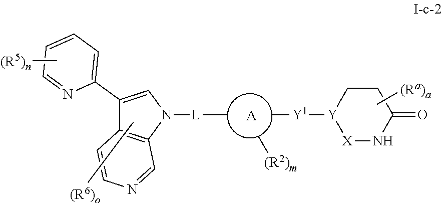

or a pharmaceutically acceptable salt thereof, wherein each of the variables is as defined in formula I-c above and described in embodiments herein, both singly and in combination.

In some embodiments, the present invention provides the compound of formula I-c as a compound of formula I-c-3:

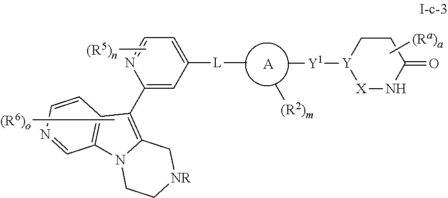

or a pharmaceutically acceptable salt thereof, wherein each of the variables is as defined in formula I-c above and described in embodiments herein, both singly and in combination.

In some embodiments, the present invention provides the compound of formula I-c as a compound of formula I-c-4:

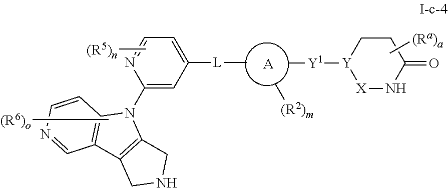

or a pharmaceutically acceptable salt thereof, wherein each of the variables is as defined in formula I-c above and described in embodiments herein, both singly and in combination.

As defined herein and described below, wherein a formula is depicted using square brackets, e.g.,

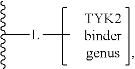

L is attached to a modifiable carbon, oxygen, or nitrogen atom within the referenced TYK2 binder genus including substitution or replacement of a defined variable within the referenced TYK2 binder genus.

In some embodiments, the present invention provides a compound of formula II-a:

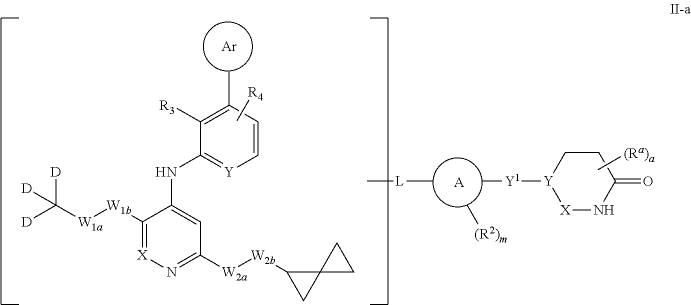

or a pharmaceutically acceptable salt thereof, wherein each of the variables in

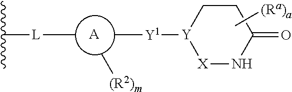

is as defined in the formulae above (e.g., formula I-a) and described in embodiments herein, both singly and in combination, and wherein:
Ar is a five-membered heteroaryl ring; and the five-membered heteroaryl ring is optionally substituted by 1, 2, 3 or 4 substituents selected from the group consisting of halogen, substituted or unsubstituted C₁₋₆ alkyl, substituted or unsubstituted C₁₋₆ hydroxyalkyl, substituted or unsubstituted C₁₋₄ alkylene-O—C₁-6 alkyl, substituted or unsubstituted C₁₋₄ alkylene-S—C₁₋₆ alkyl;
Y is CH or N;
X is CH or N;
R₃ is selected from the group consisting of H, halogen, substituted or unsubstituted C₁₋₆ alkyl, hydroxyl, substituted or unsubstituted C₁₋₆ alkoxy, substituted or unsubstituted C₁₋₆ alkylthio;
R⁴ does not exist, or represents 1 or 2 substituents selected from the group consisting of halogen, substituted or unsubstituted C₁₋₃ alkyl;
W₁ₐ and W_{1b} are each independently selected from the group consisting of a covalent bond, —O—, —S—, —SO₂—, —CO—, —NR₅—;
W₂ₐ and W_{2b} are each independently selected from the group consisting of a covalent bond, —O—, —S—, —SO₂—, —CO—, —NR₅—;
R₅ are each independently selected from H, substituted or unsubstituted C₁₋₄ alkyl; or one or more of the variables are as defined in WO 2023/030335, the entirety of which is herein incorporated by reference.

In some embodiments, the present invention provides a compound of formula II-b:

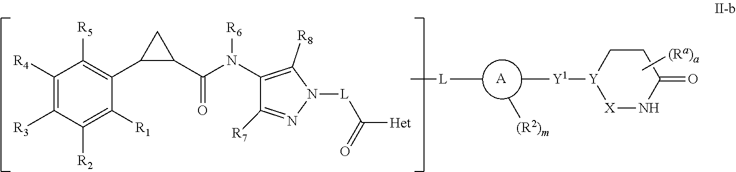

or a pharmaceutically acceptable salt thereof, wherein each of the variables in

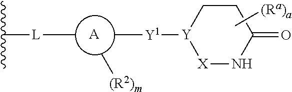

is as defined in the formulae above (e.g., formula I-a) and described in embodiments herein, both singly and in combination, and wherein:
R₁ to R₅ each independently are selected from H, hydroxy, C₁₋₆ alkoxy, —CO₂C₁₋₆ alkyl; R₆ is H or C₁₋₆ alkyl;
R₇ and R₈ are each independently selected from H, halogen, cyano, nitro, hydroxy, amino, carboxyl, C₁₋₆ alkyl, C₃₋₈ cycloalkyl, C₁₋₆ haloalkyl, C₁₋₆ alkoxy, C₁₋₆ alkylthio, C₁₋₄ alkoxy, C₁₋₄ alkyl, C₁₋₆ alkylsulfonyl, C₁₋₆ alkylamino, di(C₁₋₆ alkyl)amino, C₁₋₆ alkylcarbonyl, C₁₋₄ alkoxycarbonyl;
L is C₁₋₆ alkylene;
Het is selected from an optionally substituted heterocycloalkyl group comprising at least one nitrogen atom and which is linked to the carbonyl group via the nitrogen atom;
or one or more of the variables are as defined in CN 115677676, the entirety of which is herein incorporated by reference.

In some embodiments, the present invention provides a compound of formula II-c:

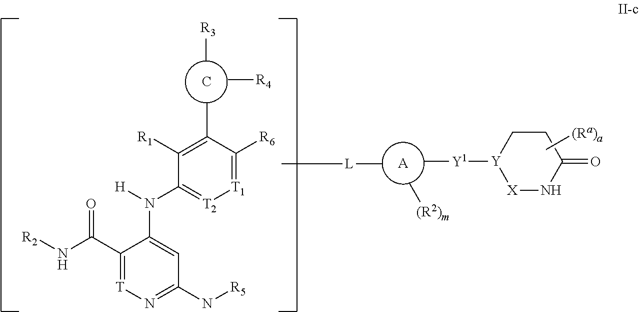

or a pharmaceutically acceptable salt thereof, wherein each of the variables in

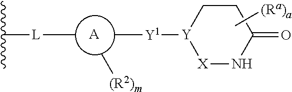

is as defined in the formulae above (e.g., formula I-a) and described in embodiments herein, both singly and in combination, and wherein:
L is selected from —NH—, —NHC(O)—, —NHC(O)O—, and —NHC(O)NH—;
T, T and T are independently selected from N and CH, the CH is optionally substituted by 1 halogen;
R₁ is selected from C₁₋₃ alkoxy, and the C₁₋₃ alkoxy is optionally substituted by 1, 2 or 3 Rₐ;
R₂ is selected from H and C₁₋₃ alkyl, said C₁₋₃ alkyl is optionally substituted by 1, 2 or 3 R_{b};
Ring C is selected from phenyl and 6-membered heteroaryl;
R₃ is selected from —P(O)(C₁₋₃ alkyl)₂, —P(O)(C₃₋₅ cycloalkyl)₂, —S(O)C₁₋₄ alkyl, —S(O)ₙC₁₋₃ alkylamino, —S(O)ₙ-4-5 membered heterocycloalkyl, —S(O)ₙNH₂, —S(O)(NR)C₁₋₄ alkyl, —S(O)(NR)C₁₋₃ alkylamino, —S(O)(NR)C₃₋₈ cycloalkyl,

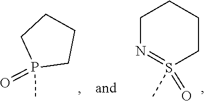

and wherein the —P(O)(C₁₋₃ alkyl)₂, —P(O)(C₃₋₅ cycloalkyl)₂, —S(O)˜C₁₋₄ alkyl, —S(O)ₙC₁₋₃ alkylamino, —S(O)n-4-5 membered heterocycloalkyl, —S(O)ₙNH₂, —S(O)(NR)C₁₋₄ alkyl, —S(O)(NR)C₁₋₃ alkylamino, —S(O)(NR)C₃₋₅ cycloalkyl,

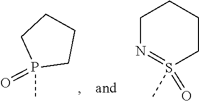

are each independently optionally substituted by 1, 2 or 3 In some embodiments, the present invention provides a compound of formula II-d: halogens;
R₅ is selected from C₁₋₃ alkyl, C₃₋₅ cycloalkyl and 5-6 membered heteroaryl, the C₁₋₃ alkyl, C₃₋₅ cycloalkyl and 5-6 membered heteroaryl independently and optionally substituted by 1, 2 or 3 R_{c};
R₄ and R₆ are independently selected from H, F, Cl, Br, and I;
alternatively, R₃ and R₄ together with the carbon atoms to which they are attached form

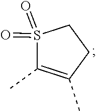

Rₐ and R_{b} are independently selected from H, D, F, Cl, Br, and I;
R_{c} is selected from F, Cl, Br, I and C₁₋₃ alkyl;
R is selected from H and C₁₋₃ alkyl;
n is 1 or 2;
or one or more of the variables are as defined in WO 2023/284869, the entirety of which is herein incorporated by reference.

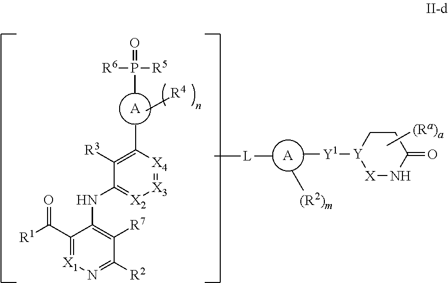

or a pharmaceutically acceptable salt thereof, wherein each of the variables in

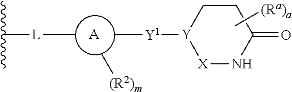

is as defined in the formulae above (e.g., formula I-a) and described in embodiments herein, both singly and in combination, and wherein:
n is 0, 1, 2 or 3;
X₁ is N or CH;
each of X₂, X₃ and X₄ is independently N or CR⁸;
Ring A is C₆₋₁₀ aryl or 5-10 membered heteroaryl;
R¹ is C₁₋₆ alkyl, C₁₋₆ deuterated alkyl, C₂₋₆ alkenyl, C₂₋₆ alkynyl, C₃₋₆ cycloalkyl, —NH(deuterated C₁₋₆ alkyl), or —NH(C₁₋₆ alkyl), wherein C₁₋₆ alkyl, C₁₋₆ deuterated alkyl, C₂₋₆ alkenyl, C₂₋₆ alkynyl, and C₃₋₆ cycloalkyl) are unsubstituted or substituted with one or more groups independently selected from R^{aa};
preferably R¹ is independently C₁₋₆ alkyl, C₁₋₆ deuterated alkyl, C₂₋₆ alkenyl, C₂₋₆ alkynyl, or C₃₋₆ cycloalkyl, wherein C₁₋₆ alkyl, C₁₋₆ deuterated alkyl, C₂₋₆ alkenyl, C₂₋₆ alkynyl, or C₃₋₆ cycloalkyl, are unsubstituted or substituted with one or more groups independently selected from R^{aa};
R⁸⁸ is hydrogen, deuterium, halide, amino, —NO₂, —CN, —OH, or C₁₋₃ alkyl;
R² is alkyl, deuterated alkyl, halo-alkyl, alkoxy, halo-alkoxy, alkenyl, alkynyl, cycloalkyl, heterocycloalkyl, aryl, heteroaryl, —NR^{b}R^{c}, —C(O)R^{a}, —C(O)NR^{b}R^{c}, —S(O)R^{a}, —S(O)₂R^{a}, —C(O)OR^{a}, —NR^{d}C(O)R^{a}, —NR^{d}C(O)NR^{b}R^{c}, —NR^{d}S(O)R^{a}, —NR^{d}S(O)₂R^{a}, —NRS(O)NR^{b}R^{c}, —NR^{d}S(O)₂NR^{c}R^{d}, or —NR^{d}C(O)OR^{a}, wherein alkyl, deuterated alkyl, halo-alkyl, alkoxy, halo-alkoxy, alkenyl, alkynyl, cycloalkyl, heterocycloalkyl, aryl, and heteroaryl are unsubstituted or substituted with one or more groups independently selected from hydrogen, deuterium, halide, amino, —NO₂, —CN, —OH, C₁₋₃ alkyl, deuterated C₁₋₃ alkyl, C₁₋₃ halo-alkyl, C₁₋₃ alkoxy, deuterated C₁₋₃ alkoxy, C₁₋₃ halo-alkoxy, C₂₋₆ alkenyl, C₂₋₆ alkynyl, substituted or unsubstituted cycloalkyl, substituted and unsubstituted heterocycloalkyl, and substituted or unsubstituted aryl; R³ is hydrogen, halide, —OH, amino, —SH, —NO₂, —CN, C₁₋₆ alkyl, —C(O)NH₂, C₁₋₆ deuterated alkyl, —O(C₁₋₆ alkyl), —O(C₁₋₆ deuterated alkyl), C₂₋₆ alkenyl, C₂₋₆ alkynyl, C₃₋₆ cycloalkyl, C₃₋₆ heterocycloalkyl, C₆₋₁₀ aryl or 5-10 membered heteroaryl, wherein C₁₋₆ alkyl, C₁₋₆ deuterated alkyl, —O(C₁₋₆ alkyl), —O(C₁₋₆ deuterated alkyl), C₂₋₆ alkenyl, C₂₋₆ alkynyl, C₃₋₆ cycloalkyl, C₃₋₆ heterocycloalkyl, C₆₋₁₀ aryl and 5-10 membered heteroaryl are unsubstituted or substituted with one or more groups independently selected from R^{aa};
if present, each R⁴ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
each of RI and R⁶ is independently C₁₋₆ alkyl, C₁₋₆ deuterated alkyl, C₂₋₆ alkenyl, C₂₋₆ alkynyl, or C₃₋₆ cycloalkyl, wherein C₁₋₆ alkyl, C₁₋₆ deuterated alkyl, C₂₋₆ alkenyl, C₂₋₆ alkynyl, and C₃₋₆ cycloalkyl are unsubstituted or substituted with one or more groups selected from R^{bb};
preferably each of R⁵ and R⁶ is independently C₁₋₃ alkyl, wherein C₁₋₃ alkyl is unsubstituted or substituted with one or more groups selected from R^{bb}; or R⁵ and R⁶, together with P attached thereto, form a 5-6 membered heterocycloalkyl, wherein 5-6 membered heterocycloalkyl is unsubstituted or substituted with one or more groups selected from R^{bb};
R⁷ is hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
if present, each R⁸ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb}; and
each of R^{a}, R^{b}, R^{c} and R^{d} is independently hydrogen, deuterium, halide, amino, —NO₂, —CN, —OH, alkyl, deuterated alkyl, halo-alkyl, alkoxy, halo-alkoxy, alkenyl, alkynyl, cycloalkyl, heterocycloalkyl, aryl, or heteroaryl, wherein alkyl, deuterated alkyl, halo- alkyl, alkoxy, halo-alkoxy, alkenyl, alkynyl, cycloalkyl, heterocycloalkyl, aryl, and heteroaryl are unsubstituted or substituted with one or more groups independently selected from hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted cycloalkyl, and substituted and unsubstituted heterocycloalkyl, substituted or unsubstituted aryl, and substituted or unsubstituted heteroaryl; or
   any two of adjacent or non-adjacent R^{a}, R^{b}, R^{c} and R^{d} form a cycloalkyl, heterocycloalkyl, aryl or heteroaryl, wherein cycloalkyl, heterocycloalkyl, aryl and heteroaryl are unsubstituted or substituted with one or more groups selected from hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted 6-10 membered aryl, and substituted or unsubstituted 5-10 membered heteroaryl;
or one or more of the variables are as defined in WO 2022/261524, the entirety of which is herein incorporated by reference.

In some embodiments, the present invention provides a compound of formula II-e:

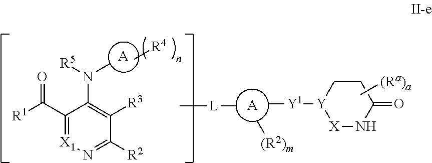

or a pharmaceutically acceptable salt thereof, wherein each of the variables in

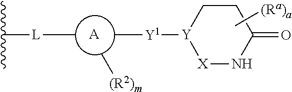

is as defined in the formulae above (e.g., formula I-a) and described in embodiments herein, both singly and in combination, and wherein:
n is selected from 0, 1, 2 and 3; X₁ selected from N and CH;
Ring A is selected from C₆₋₁₀ aryl and 5 to 10 membered heteroaryl;
R¹ is selected from amino, C₁₋₆ alkyl, C₁₋₆ deuterated alkyl, C₂₋₆ alkenyl, C₂₋₆ alkynyl, C₃₋₆ cycloalkyl and —NHC₁₋₆ alkyl, said C₆ alkyl, C₁₋₆ deuterated alkyl, C₂₋₆ alkenyl, C₂₋₆ alkynyl, C₃₋₆ cycloalkyl and —NHC₁₋₆ alkyl are each independently selected from hydrogen, deuterium, halogen, amino, nitro, cyano, hydroxy and C₁₋₃ alkyl substituted by one or more substituents in the alkyl group;
R² is selected from the group consisting of alkyl, deuterated alkyl, haloalkyl, alkoxy, haloalkoxy, alkenyl, alkynyl, cycloalkyl, heterocycloalkyl, aryl, heteroaryl, —NR^{c}R^{b}, —C(O)R^{a}, —C(O)NR^{c}R^{b}, —S(O)R^{a}, —S(O)₂R^{a}, —C(O)OR^{a}, —NR^{d}C(O)R^{a}, —NR^{d}C(O)NR^{c}R^{d}, —NR^{d}S(O)R^{a}, —NR^{d}S(O)₂R^{a}, —NR^{d}S(O)NR^{b}R^{c}, —NR^{d}S(O)₂NR^{b}R^{c}, and —NR^{d}C(O)OR^{a}, wherein the alkyl, deuterated alkyl, haloalkyl, alkoxy, haloalkoxy, alkenyl, alkynyl, cycloalkyl, heterocycloalkyl, aryl, and heteroaryl are each independently substituted with one or more substituents selected from hydrogen, deuterium, halogen, amino, nitro, cyano, hydroxyl, alkyl, deuterated alkyl, haloalkyl, alkoxy, haloalkoxy, alkenyl, alkynyl, substituted or unsubstituted cycloalkyl, substituted or unsubstituted heterocycloalkyl, substituted or unsubstituted aryl, and substituted or unsubstituted heteroaryl;
R³ is selected from hydrogen, deuterium, halogen, hydroxy, amino, cyano, trifluoromethyl, C₁₋₆ alkyl, C₃₋₆ cycloalkyl, —OC₁₋₆ alkyl, —NHC₁₋₆ alkyl, —N(C₁₋₆ alkyl)₂, C₂₋₆ alkenyl, and C₂₋₆ alkynyl, said C₁₋₆ alkyl, C₃₋₆ cycloalkyl, C₂₋₆ alkenyl and C₂₋₆ alkynyl, wherein each group is independently substituted with one or more substituents selected from hydrogen, deuterium, halogen, amino, nitro, cyano and hydroxy;
if present, each R⁴ is each independently selected from hydrogen, deuterium, halogen, hydroxy, amino, mercapto, nitro, cyano, C₁₋₆ alkyl, C₁₋₆ deuterated alkyl, —OC₁₋₆ alkyl, —OC₁₋₆ deuterated alkyl, —NHC₁₋₆ alkyl, —N(C₁₋₆ alkyl)₂, C₂₋₆ alkenyl, and C₂₋₆ alkynyl, C₃₋₆ cycloalkyl, C₃₋₆ heterocycloalkyl, P(O)R₅R⁶, C₆₋₁₀ aryl, and C₁₋₁₀ heteroaryl, of said C₁₋₆ alkyl, C₁₋₆ deuterated alkyl, —OC₁₋₆ alkyl, —OC₁₋₆ deuterated alkyl, C₂₋₆ alkenyl, C₂₋₆ alkynyl, C₃₋₆ cycloalkyl, C₃₋₆ heterocycloalkyl, C₆₋₁₀ aryl and C₁₋₁₀ heteroaryl is each independently selected from hydrogen, deuterium, halogen, amino, nitro, cyano, hydroxy and C₁₋₃ alkyl substituted with one or more substituents in the alkyl group;
if present, R⁵ and R⁶ are each independently selected from C₁₋₃ alkyl, said C₁₋₃ alkyl is substituted with one or more substituents selected from hydrogen, deuterium, halogen, amino, nitro, cyano and hydroxy; or R⁵ and R⁶ together with the phosphorus atom to which they are attached form a 5 to 6-membered heterocycloalkyl, said 5 to 6-membered heterocycloalkyl being substituted with one or more substituents selected from the group consisting of hydrogen, deuterium, halogen, amino, nitro, cyano and hydroxyl;
R⁸ is selected from hydrogen and C₁₋₆ alkyl; or R⁸ and R⁴ together with ring a form a 5 to 10-membered heterocyclic ring;
R^{a}, R^{b}, R^{c}, and R^{d} are each independently selected from the group consisting of hydrogen, deuterium, halogen, amino, nitro, cyano, hydroxy, alkyl, deuterated alkyl, haloalkyl, alkoxy, haloalkoxy, alkenyl, alkynyl, cycloalkyl, heterocycloalkyl, aryl, and heteroaryl, wherein said alkyl, deuterated alkyl, haloalkyl, alkoxy, haloalkoxy, alkenyl, alkynyl, cycloalkyl, heterocycloalkyl, aryl, and heteroaryl are each independently selected from the group consisting of hydrogen, deuterium, halogen, amino, nitro, and heteroaryl, cyano, hydroxy, alkyl, deuterated alkyl, haloalkyl, alkoxy, haloalkoxy, alkenyl, alkynyl, substituted or unsubstituted cycloalkyl, substituted or unsubstituted heterocycloalkyl, substituted or unsubstituted aryl, and substituted or unsubstituted heteroaryl; or
   any two adjacent or non-adjacent R^{a}, R^{b}, R^{c}, and R^{d} are joined to form a cycloalkyl, heterocycloalkyl, aryl, or heteroaryl group, wherein said cycloalkyl, heterocycloalkyl, aryl, or heteroaryl group is substituted with one or more substituents selected from the group consisting of hydrogen, deuterium, halogen, amino, nitro, cyano, hydroxy, alkyl, deuterated alkyl, haloalkyl, alkoxy, haloalkoxy, alkenyl, alkynyl, cycloalkyl, heterocycloalkyl, aryl, and heteroaryl;
or one or more of the variables are as defined in CN 115466257, the entirety of which is herein incorporated by reference.

In some embodiments, the present invention provides a compound of formula II-f:

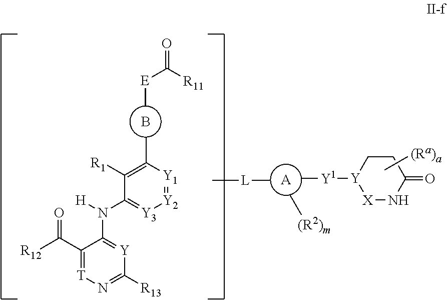

or a pharmaceutically acceptable salt thereof, wherein each of the variables in

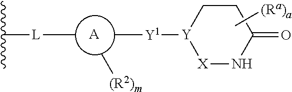

is as defined in the formulae above (e.g., formula I-a) and described in embodiments herein, both singly and in combination, and wherein:
T, Y, Y₁, Y₂ and Y₃ are selected from N and CH, said CH is optionally substituted by 1 halogen, C₁₋₃ alkyl and C₁₋₃ alkoxy;
R₁₁ is selected from C₂₋₄ alkenyl, C₂₋₄ alkynyl and CH₂CN, and the C₂₋₄ alkenyl, C₂₋₄ alkynyl and CH₂CN are optionally replaced by 1, 2, or 3 halogen substitution;
R₁₂ is selected from —N(R₂)₂ and —C(R₂)₃;
R₁₃ is selected from C₃₋₆ cycloalkyl, 3-6 membered heterocycloalkyl, phenyl, 5-6 membered heteroaryl, —NHC(O)C₃₋₆ cycloalkyl, —NHC(O)-3-6 membered heterocycloalkyl, —NHC(O)phenyl and —NHC(O)-5-6 membered heteroaryl, the C₃₋₆ cycloalkyl, 3-6 membered heterocyclic alkyl, phenyl, 5-6 membered heteroaryl, —NHC(O)C₃₋₆ cycloalkyl, —NHC(O)-3-6 membered heterocycloalkyl, —NHC(O)-phenyl and —NHC(O)-5-6 membered heteroaryl optionally substituted by 1, 2 or 3 halogens, C₁₋₃ alkyl and C₁₋₃ alkoxy;
R₁ is selected from C₁₋₃ alkyl and C₁₋₃ alkoxy, said C₁₋₃ alkyl and C₁₋₃ alkoxy are optionally substituted by 1, 2, or 3 Rₐ;
R₂ is selected from H, C₁₋₃ alkyl and C₁₋₃ alkoxy, said C₁₋₃ alkyl and C₁₋₃ alkoxy are optionally substituted by 1, 2, or 3 R_{b};
Ring B does not exist;
alternatively, Ring B is selected from C₅₋₁₀ cycloalkyl, 5-10 membered heterocycloalkyl, C₆₋₁₀ aryl and 5-10 membered heteroaryl, the C₅₋₁₀ cycloalkyl, 5-10 membered heterocycloalkyl, C₆₋₁₀ aryl and 5-10 membered heteroaryl are optionally substituted by 1, 2, or 3 R_{c};
E is selected from —(CH₂)ₛNR₃—,

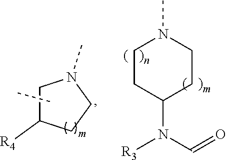

and —C(O)NR₃—C₃₋₆ cycloalkyl-NR₃—;
s is 0, 1, or 2;
n and m are independently selected from 0, 1, 2, and 3;
R₃ are independently selected from H and C₁₋₃ alkyl;
each Rₐ is independently selected from F, Cl, Br, I and OH;
each R_{b} is independently selected from H, D, F, Cl, Br and I;
each R_{c} is independently selected from H, F, Cl, Br, I and C₁₋₃ alkyl, and the C₁₋₃ alkyl is optionally substituted by 1, 2, or 3 halogens
or one or more of the variables are as defined in WO 2022/253333, the entirety of which is herein incorporated by reference.

In some embodiments, the present invention provides a compound of formula II-g:

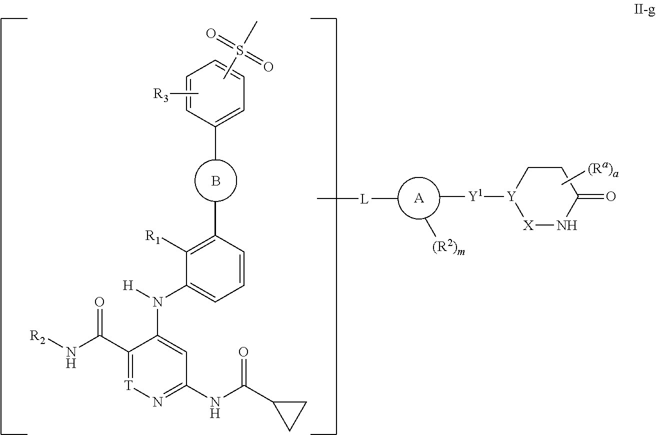

or a pharmaceutically acceptable salt thereof, wherein each of the variables in

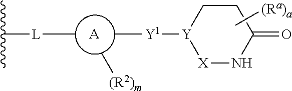

is as defined in the formulae above (e.g., formula I-a) and described in embodiments herein, both singly and in combination, and wherein:
T is selected from N and CH optionally substituted by 1 halogen;
R₁ is selected from C₁₋₃ alkyl and C₁₋₃ alkoxy, said C₁₋₃ alkyl and C₁₋₃ alkoxy are optionally substituted by 1, 2, or 3 Rₐ;
R₂ is selected from H, C₁₋₃ alkyl and C₁₋₃ alkoxy, said C₁₋₃ alkyl and C₁₋₃ alkoxy are optionally substituted by 1, 2, or 3 R^{b};
R₃ is selected from F, Cl, Br, I and C₁₋₃ alkyl;
Ring B is selected from 5-membered heteroaryl optionally substituted by 1, 2, or 3 R_{c};
Rₐ and R^{b} are independently selected from H, D, F, Cl, Br and I;
each R_{c} is independently selected from H, F, Cl, Br, I and C₁₋₃ alkyl, and the C₁₋₃ alkyl is optionally substituted by 1, 2 or 3 R;
each R is selected from F, Cl, Br and I;
or one or more of the variables are as defined in WO 2022/253335, the entirety of which is herein incorporated by reference.

In some embodiments, the present invention provides a compound of formula II-h:

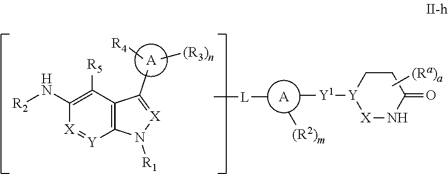

or a pharmaceutically acceptable salt thereof, wherein each of the variables in

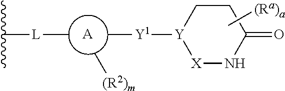

is as defined in the formulae above (e.g., formula I-a) and described in embodiments herein, both singly and in combination, and wherein:
Ring A is selected from aryl, heteroaryl or partially unsaturated heterocyclic;
X, Y are selected from C or N;
R₁ is selected from hydrogen, C₁-C₆ alkyl, C₂-C₆ alkenyl, C₂-C₆ alkynyl, halogenated C₁-C₆ alkyl, deuterated C₁-C₆ alkyl, halogenated C₂-C₆ alkenyl, halogenated C₂-C₆ alkynyl, halogen, cyano, nitro, —C(O)NRₐR_{b}, —C(O)R^{a}, —C(O)ORₐ, —OR, —RₐOR_{b}, —OC(O)Rₐ, —OC(O)ORₐ, —OC(O)NRₐR_{b}, —NRₐR_{b}, —SRₐ, —S(O)Rₐ, —S(O)₂R^{a} or a 3-10 membered saturated or unsaturated ring containing 0-3 heteroatoms, said 3-10 membered saturated ring containing 0-3 heteroatoms Or the unsaturated ring is optionally substituted by 1-3 Rₐ;
R₂ is selected from hydrogen, C₁-C₆ alkyl, C₂-C₆ alkenyl, C₂-C₆ alkynyl, halogenated C₁-C₆ alkyl, deuterated C₁-C₆ alkyl, halogenated C₂-C₆ alkenyl, halogenated C₂-C₆ alkynyl, halogen, cyano, nitro, —C(O)NRₐR, —C(O)R^{a}, —C(O)ORₐ, —OR, —RₐOR_{b}, —OC(O)Rₐ, —OC(O)ORₐ, —OC(O)NRₐR_{b}, —NRₐR_{b}, —SRₐ, —S(O)Rₐ, —S(O)₂Rₐ or a 3-10 membered saturated or unsaturated ring containing 0-3 heteroatoms, said 3-10 membered saturated ring containing 0-3 heteroatoms or the unsaturated ring is optionally substituted by 1-3 Rₐ;
R₃ is selected from hydrogen, C₁-C₆ alkyl, C₂-C₆ alkenyl, C₂-C₆ alkynyl, halogenated C₁-C₆ alkyl, deuterated C₁-C₆ alkyl, halogenated C₂-C₆ alkenyl, halogenated C₂-C₆ alkynyl, halogen, cyano, nitro, carbonyl, —C(O)NRₐR_{b}, —C(O)R^{a}, —C(O)OR, —OR, —RₐOR_{b}, —ORₐOR_{b}, —OC(O)R, —OR, —C(O)R_{b}, —OC(O)ORₐ, —OC(O)NRₐR_{b}, —NRₐR_{b}, —NC(O)R^{a}, —R_{c}NRₐR_{b}, —SR, —S(O)R^{a}, —S(O)₂R, —RₐCN or a 3-10 membered saturated or unsaturated ring containing 0-3 heteroatoms, the 3-10 membered saturated or unsaturated ring containing 0-3 heteroatoms is optionally replaced by 1-3 Rₐ;
R₄ is selected from hydrogen,

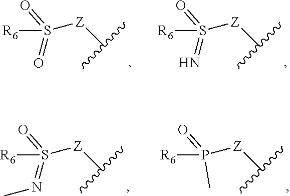

or a 3-10 membered saturated or unsaturated ring containing 0-3 heteroatoms optionally substituted by 1-3 Rₐ, Z is —NH—, —N(CH₂)— or a direct bond;
R₅ is selected from hydrogen, C₁-C₆ alkyl, C₂-C₆ alkenyl, C₂-C₆ alkynyl, halogenated C₁-C₆ alkyl, deuterated C₁-C₆ alkyl, halogenated C₂-C₆ alkenyl, halogenated C₂-C₆ alkynyl, halogen, cyano, nitro, —C(O)NRₐR_{b}, —C(O)Rₐ, —C(O)ORₐ, —ORₐ, —RₐOR_{b}, —OC(O)Rₐ, —OC(O)ORₐ, —OC(O)NRₐR_{b}, —NRₐR^{b}, —SR, —S(O)R^{a}, —S(O)₂R^{a} or a 3-10 membered saturated or unsaturated ring containing 0-3 heteroatoms, said 3-10 membered saturated ring containing 0-3 heteroatoms or the unsaturated ring is optionally substituted by 1-3 Rₐ;
R₆ is selected from hydrogen, C₁-C₆ alkyl, C₂-C₆ alkenyl, C₂-C₆ alkynyl, halogenated C₁-C₆ alkyl, deuterated C₁-C₆ alkyl, halogenated C₂-C₆ alkenyl, halogenated C₂-C₆ alkynyl, halogen, cyano, nitro, —C(O)NRₐR_{b}, —C(O)Rₐ, —C(O)ORₐ, —OR, —RₐOR_{b}, —OC(O)Rₐ, —OC(O)ORₐ, —OC(O)NRₐR_{b}, —NRₐR^{b}, —SR^{a}, —S(O)R, —S(O)₂R^{a} or a 3-10 membered saturated or unsaturated ring containing 0-3 heteroatoms, said 3-10 membered saturated ring containing 0-3 heteroatoms or the unsaturated ring is optionally substituted by 1-3 Rₐ;
each Rₐ, R_{b}, R_{c} is independently selected from hydrogen, deuterium, C₁-C₆ alkyl, C₂-C₆ alkenyl, C₂-C₆ alkynyl, halogenated C₁-C₆ alkyl, deuterated C₁-C₆ alkyl, halogenated C₂-C₆ alkenyl, halogenated C₂-C₆ alkynyl, halogen, cyano, nitro, amino, carboxyl, carbonyl, hydroxy, hydroxy alkyl, alkoxy, haloalkoxy, deuterated alkoxy, C₃-C₆ cycloalkyl, haloC₃-C₆ cycloalkyl, alkoxy substituted C₃-C₆ cycloalkyl, C₃-C₆ heterocyclyl, halogenated C₃-C₆ heterocyclyl, alkyl substituted C₃-C₆ heteroaryl, —S(O)₂R_{d}, —OR_{d}ORₑ, —R_{d}ORₑ, —C(O)R_{d} or —OC(O)R_{d}, each R_{d}, Rₑ is independently selected from C₁-C₆ alkyl; and
n is 0, 1, 2 or 3;
or one or more of the variables are as defined in WO 2022/242697, the entirety of which is herein incorporated by reference.

In some embodiments, the present invention provides a compound of formula II-i:

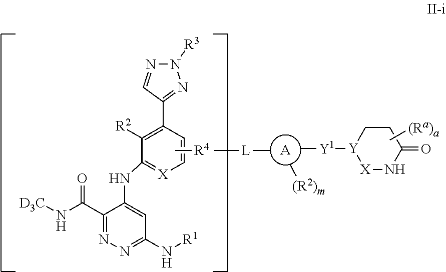

or a pharmaceutically acceptable salt thereof, wherein each of the variables in

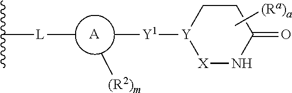

is as defined in the formulae above (e.g., formula I-a) and described in embodiments herein, both singly and in combination, and wherein:
X is —N— or —CH—;
R¹ is —C(O)R^{1a}; or a 5-8 membered heterocycle containing 1-2 heteroatoms selected from N, O, and S, each heterocycle substituted with 0-2 R^{1b};

R^{1a} is COOC₃ alkyl, or C₃₋₆ cycloalkyl, said cycloalkyl group substituted with 0-2 R^{1b}; R^{1b} is independently at each occurrence, F or C₃ alkyl;
R² is OMe or OCHF₂;
R³ is CD₃, C₃ alkyl, C₃₋₆ cycloalkyl or CH₂F; and
R⁴ is hydrogen, halo, C₁₋₄ alkyl, C₁₋₄ alkoxy or C₃₋₆ cycloalkyl;
or one or more of the variables are as defined in WO 2022/241171, the entirety of which is herein incorporated by reference.

In some embodiments, the present invention provides a compound of formula II-j:

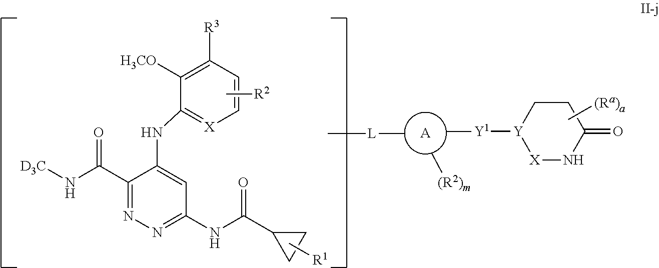

or a pharmaceutically acceptable salt thereof, wherein each of the variables in

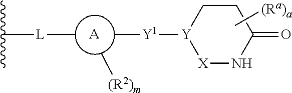

is as defined in the formulae above (e.g., formula I-a) and described in embodiments herein, both singly and in combination, and wherein:
X is —N— or —CH—;
R¹ is C₁₋₃ alkyl;
R² is F or C₁₋₃ alkyl;
R³ is oxadiazol, oxazol, pyridine, pyrimidine, thiadiazol, pyrazine, thiazol, pyrazol or isothiazole, all of which are substituted with 0-3 R^{3a} groups;
R^{3a} is H, F, C₁, CN, NO₂, C₁₋₃ alkyl, 0-C_{M} alkyl, C₃₋₆ cycloalkyl, (CH₂)ₙF, CHF₂, hydroxy C₃ alkyl or cyano C₁₋₃ alkyl; and
n is 1 or 2
or one or more of the variables are as defined in WO 2022/241172, the entirety of which is herein

### INCORPORATED BY REFERENCE

In some embodiments, the present invention provides a compound of formula II-k:

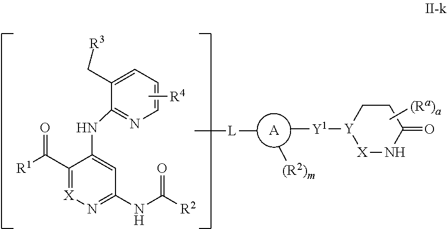

or a pharmaceutically acceptable salt thereof, wherein each of the variables in

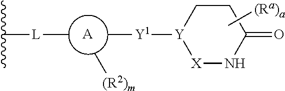

is as defined in the formulae above (e.g., formula I-a) and described in embodiments herein, both singly and in combination, and wherein:
X is —N— or —CH—;
R¹ is C₁₋₃ alkyl or —NHCD₃;
R² is —N(CH₃)₂, —OR^{2a} or C₃₋₆ cycloalkyl substituted with 0-2 R^{2b};
R^{2a} is C₁₋₃ alkyl; R^{2b} is F or C₁₋₃ alkyl; R³ is C₁₋₃ fluoroalkyl or C₃₋₆ cycloalkyl; and
R⁴ is hydrogen, halogen or C₁₋₃ alkyl
or one or more of the variables are as defined in WO 2022/241173, the entirety of which is herein incorporated by reference.

In some embodiments, the present invention provides a compound of formula II-1:

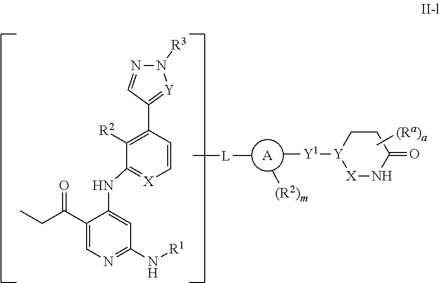

or a pharmaceutically acceptable salt thereof, wherein each of the variables in

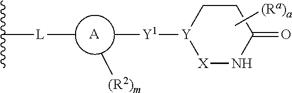

is as defined in the formulae above (e.g., formula I-a) and described in embodiments herein, both singly and in combination, and wherein:
X is —N— or —CH—;
R¹ is —C(O)R^{1a};
R^{1a} is C₃₋₆cycloalkyl;
R² is C₁₋₆ alkoxy;
R³ is C₁₋₆ alkyl or C₃₋₆ cycloalkyl;
or one or more of the variables are as defined in WO 2022/241174, the entirety of which is herein incorporated by reference.

In some embodiments, the present invention provides a compound of formula II-m:

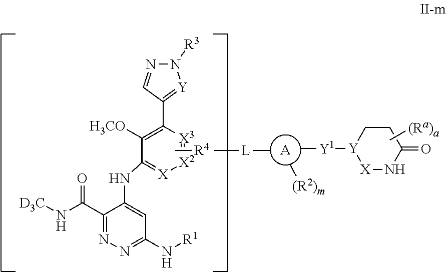

or a pharmaceutically acceptable salt thereof, wherein each of the variables in

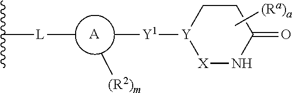

is as defined in the formulae above (e.g., formula I-a) and described in embodiments herein, both singly and in combination, and wherein:
X, X¹ and X² are —N— or —CH—, with the proviso that if X is —CH—, one of X¹ and X² must be —N—, and if X is —N—, one of X¹ and X² must be —N—;
Y is —CH— or —N—;
R¹ is —OR^{1a}, —NR^{1a}R^{1b} or R²;
R^{1a} and R^{1b} are independently hydrogen or C₁₋₃ alkyl;
R² is C₃₋₆ cycloalkyl substituted with 0-2 R^{2a};
R^{2a} is F or C₁₋₃ alkyl;
R³ is C₁₋₃ alkyl or C₃₋₆ cycloalkyl; and R⁴ is hydrogen, halogen or C₁₋₃ alkyl;
or one or more of the variables are as defined in WO 2022/241175, the entirety of which is herein incorporated by reference.

In some embodiments, the present invention provides a compound of formula II-n:

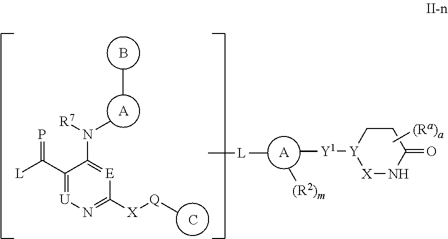

or a pharmaceutically acceptable salt thereof, wherein each of the variables in

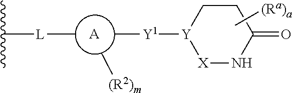

is as defined in the formulae above (e.g., formula I-a) and described in embodiments herein, both singly and in combination, and wherein:
L is alkyl, deuterated alkyl, haloalkyl, amino, alkylamino, deuterated alkylamino, cycloalkyl, cycloalkylamino, deuterated cycloalkylamino;
Ring B is aryl, heteroaryl, heterocyclyl, and Ring B is selected from the following groups:

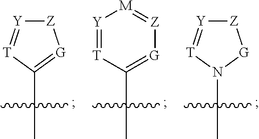

wherein, T, G, Y, Z, M are each independently selected from oxygen atom, CR^{A1}, CR^{A2}, nitrogen atom or NR^{B};
E is a nitrogen atom or a carbon atom;
R^{A1}, R^{A2} are selected from hydrogen, deuterium, C₁₋₆ alkyl, halogen, and the following structures:

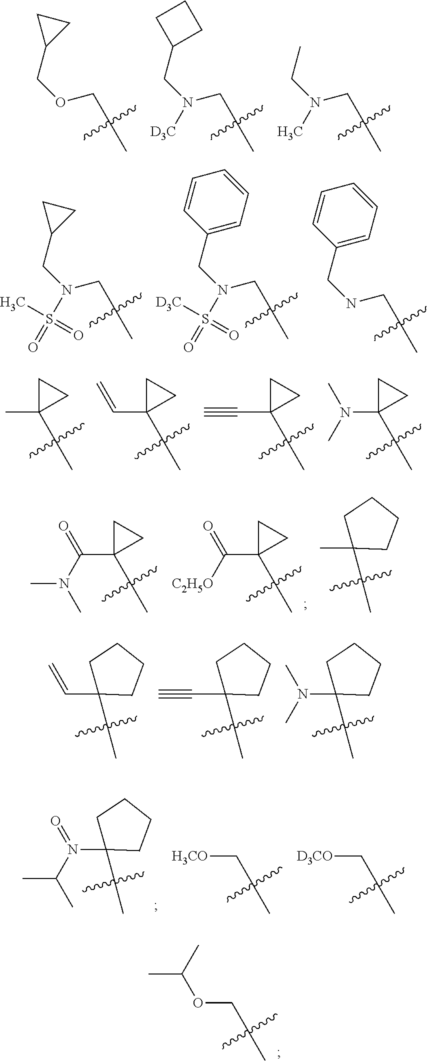

R^{B} is selected from hydrogen, C₁₋₆ alkyl, C₁₋₆ deuterated alkyl, C₁₋₆ alkyl-C(O)—, C₁₋₆ haloalkyl-C(O)—, cycloalkane base-C(O)—, aryl-C(O)—, substituted amino-C(O)—, C₁₋₆ alkyl-S(O)₂—, alkenyl, deuterated alkenyl, alkynyl, deuterated alkynyl, and the following structures:

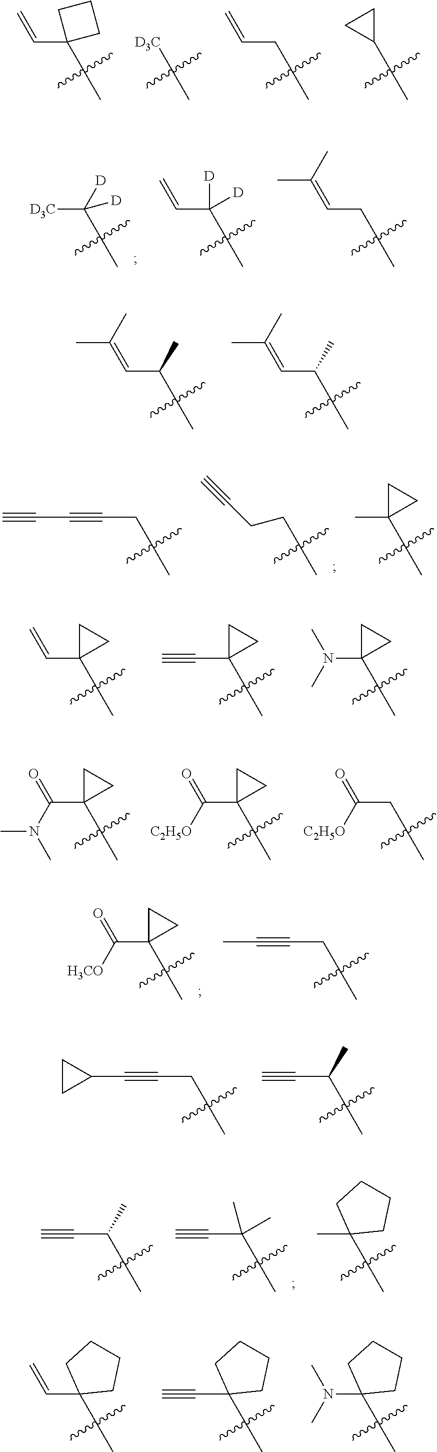

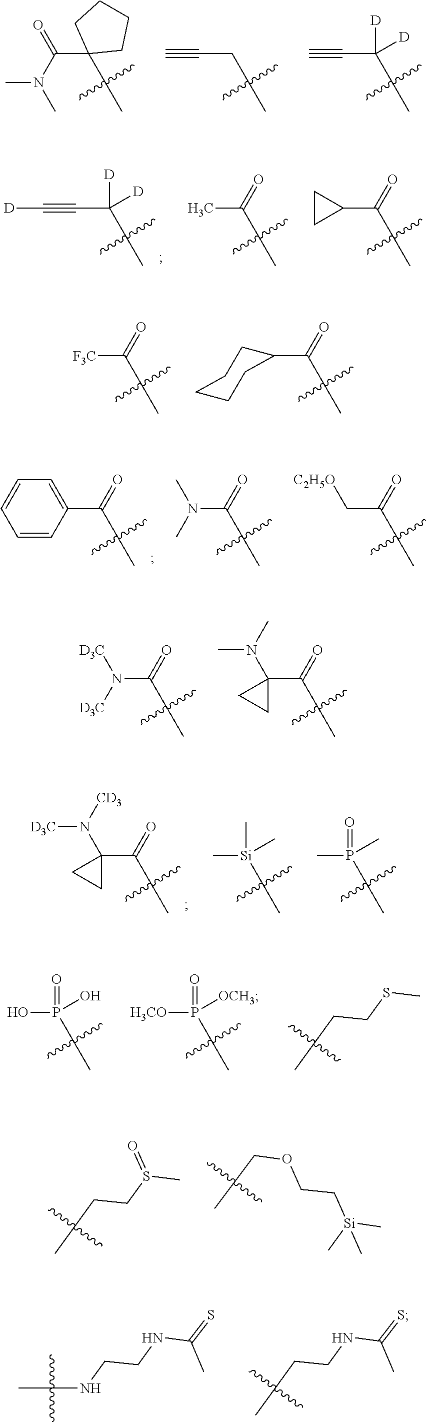

Q is a chemical bond or —C(O)—, —C(S)—, —S(O)—, —S(O)₂—, —C(NR⁸)—, that is:

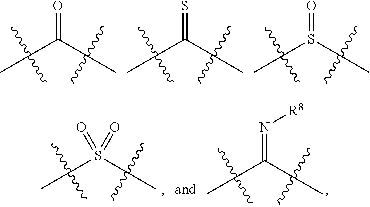

P is an oxygen atom or a sulfur atom;
X is a chemical bond, an oxygen atom, or NH or NR_{A}.
R^{A} is alkyl, deuterated alkyl, haloalkyl;
U is a nitrogen atom or a carbon atom;
Ring A is aryl, heteroaryl, heterocyclyl, and Ring A is selected from the following groups:

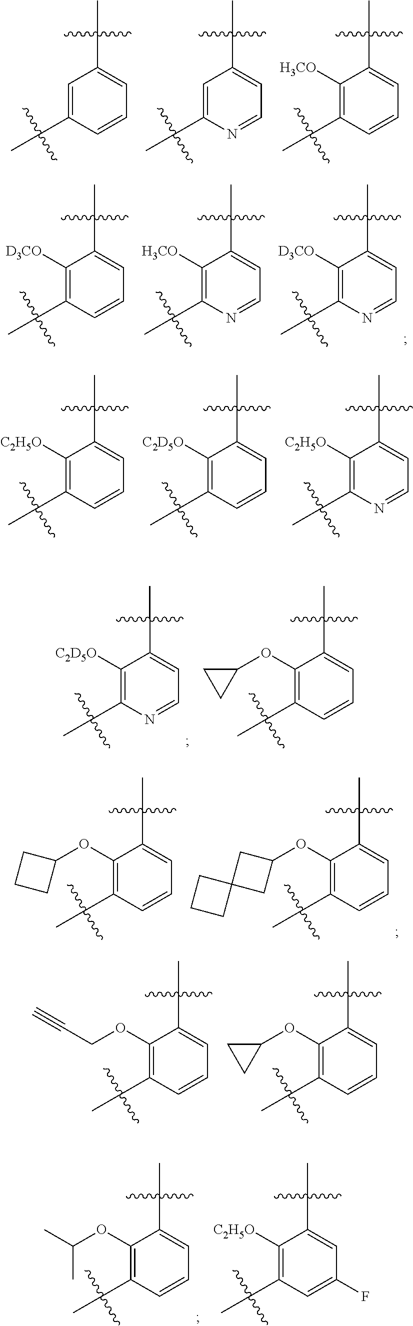

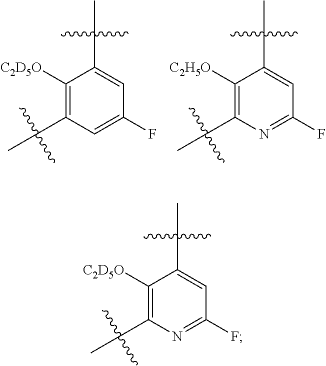

C is alkyl, cycloalkyl, amino, substituted amino, aryl, heteroaryl, heterocyclyl, and C is selected from the following groups:

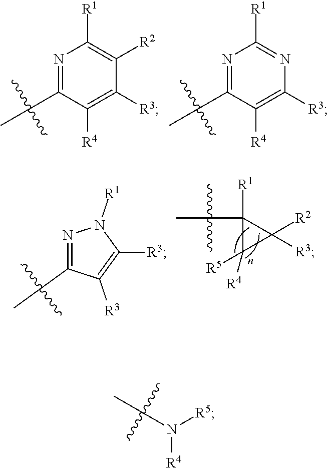

wherein, R¹, R², R³, R⁴, R⁵, R⁷, R⁸ are selected from hydrogen, deuterium, halogen, amino, alkynyl, deuterated alkynyl, alkenyl, deuterated alkenyl, alkenyl carbonyl, deuterated alkenylcarbonyl, alkyl, deuterated alkyl, alkylcarbonyl, deuterated alkylcarbonyl; where, alkynyl, alkenyl, deuterated alkynyl, deuterated alkenyl, alkyl and deuterated alkane base, optionally substituted by halogen, alkyl, hydroxyl, amino, cycloalkyl, aryl, heteroaryl; and
n is 1, 2, 3, 4, 5, 6;
or one or more of the variables are as defined in WO 2022/233286, the entirety of which is herein incorporated by reference.

In some embodiments, the present invention provides a compound of formula II-o:

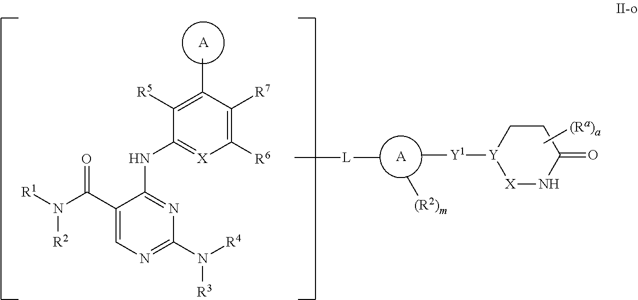

or a pharmaceutically acceptable salt thereof, wherein each of the variables in

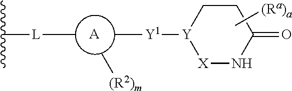

is as defined in the formulae above (e.g., formula I-a) and described in embodiments herein, both singly and in combination, and wherein:
R¹, R² each independently selected from hydrogen, deuterium, C₁₋₆ alkyl optionally substituted by halogen, C₂₋₆ alkenyl optionally substituted by halogen, C₂₋₆ alkynyl optionally substituted by halogen, C₁₋₆ alkyl optionally substituted by deuterium, C₂₋₆ alkenyl optionally substituted by deuterium, C₂₋₆ alkynyl optionally substituted by deuterium;
R³ and R⁴ are each independently selected from hydrogen, C₁₋₆ alkyl optionally substituted by halogen, C₂₋₆ alkenyl optionally substituted by halogen, C₂₋₆ alkynyl optionally substituted by halogen, —C₀₋₄ alkylene-OR^{3a}, —C₀₋₄ alkylene-OC(O)R^{3a}, —C₀₋₄ alkylene-SR^{3a}, —C₀₋₄ alkylene-S(O)₂R^{3a}, —C₀₋₄ alkylene-S(O)R^{3a}, —C₀₋₄ alkylene-S(O)₂NR^{3a}R^{3b}, —C₀₋₄ alkylene-S(O)NR^{3a}R^{3b}, —C₀₋₄ alkylene-C(O)R^{3a}, —C₀₋₄ alkylene-C(O)OR^{3a}, —C₀₋₄ alkylene-C(O)NR^{3a}R^{3b}, —C₀₋₄ alkylene-NR^{3a}R^{3b}, —C₀₋₄ alkylene-NR^{3a}C(O)R³⁶, —C₀₋₄ alkylene-(saturated or unsaturated 3 to 10-membered carbocyclic group), —C₀₋₄ alkylene-(saturated or unsaturated 4 to 10-membered heterocycloalkyl), —C₀₋₄ alkylene-(6 to 10-membered aromatic ring group), —C₀₋₄ alkylene-(5 to 10-membered aromatic heterocyclic group); wherein the alkylene, carbocyclyl, heterocycloalkyl, aromatic ring, aromatic heterocyclic may be further substituted with one, two or three independent R^{3c} substitution;
R^{3a} and R^{3b} are each independently selected from hydrogen, C₁₋₆ alkyl optionally substituted by halogen, C₂₋₆ alkenyl optionally substituted by halogen, C₂₋₆ alkynyl optionally substituted by halogen, —C₀₋₄ alkylene-(saturated or unsaturated 3 to 10-membered carbocyclic group), —C₀₋₄ alkylene-(saturated or unsaturated 4 to 10-membered heterocycloalkyl), —C₀₋₄ alkylene- (6 to 10-membered aromatic ring group), —C₀₋₄ alkylene-(5 to 10-membered aromatic) heterocyclic group); wherein the alkylene, carbocyclyl, heterocycloalkyl, aromatic ring, aromatic heterocyclic may be further substituted with one, two or three independent R^{3c} substitution;
each R^{3c} each independently selected from halogen, cyano, C₁₋₆ alkyl optionally substituted by halogen, C₂₋₆ alkenyl optionally substituted by halogen, C₂₋₆ alkynyl optionally substituted by halogen, —C₀₋₄ alkylene-OR^{3d}, —C₀₋₄ alkylene-OC(O)R^{3d}, —C₀₋₄ alkylene-SR^{3d}, —C₀₋₄ alkylene-S(O)₂R^{3d}, —C₀₋₄ alkylene-S(O)R^{3d}, —C₀₋₄ alkylene-S(O)₂NR^{3d}R³, —C₀₋₄ alkylene-S(O)NR^{3d}R^{3e}, —C₀₋₄ alkylene-C(O)R^{3d}, —C₀₋₄ alkylene-C(O)OR^{3d}, —C₀₋₄ alkylene-C(O)NR^{3d}R^{3e}, —C₀₋₄ alkylene-NR^{3d}R^{3e}, —C₀₋₄ alkylene-NR^{3d}C(O)R^{3e}, —C₀₋₄ Alkylene-(saturated or unsaturated 3 to 10-membered carbocyclic group), —C₀₋₄ alkylene-(saturated or unsaturated 4- to 10-membered heterocycloalkyl), —C₀₋₄ alkylene-(6 to 10-membered aromatic ring group), —C₀₋₄ alkylene-(5 to 10-membered aromatic heterocyclic group); wherein the alkylene, carbocyclyl, heterocycloalkyl, aromatic ring, aromatic heterocyclic may be further substituted with one, two or three independent R³ substitution; or, two independent R^{3c} together with the linking atom form

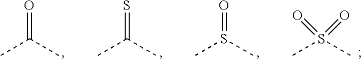

R^{3d} and R^{3e} are each independently selected from hydrogen, C₁₋₆ alkyl optionally substituted by halogen, C₂₋₆ alkenyl optionally substituted by halogen, C₂₋₆ alkynyl optionally substituted by halogen, —C₀₋₄ alkylene-(saturated or unsaturated 3 to 10-membered carbocyclic group), —C₀₋₄ alkylene-(saturated or unsaturated 4 to 10-membered heterocycloalkyl), —C₀₋₄ alkylene-(6 to 10-membered aromatic ring group), —C₀₋₄ alkylene-(5 to 10-membered aromatic heterocyclic group); or
R^{3d} and R^{3e} together with the linking atoms form a saturated or unsaturated 4 to 10-membered heterocycloalkyl group;
each R³ are independently selected from halogen, cyano, C₁₋₆ alkyl optionally substituted by halogen, C₂₋₆ alkenyl optionally substituted by halogen, C₂₋₆ alkynyl optionally substituted by halogen;
x is selected from N or CR^{x};
R⁵ is selected from hydrogen, halogen, cyano, C₁₋₆ alkyl optionally substituted by halogen, C₂₋₆ alkenyl optionally substituted by halogen, C₂₋₆ alkynyl optionally substituted by halogen, C₁₋₆ alkyl optionally substituted by deuterium, C₂₋₆ alkenyl optionally substituted by deuterium, C₂₋₆ alkynyl optionally substituted by deuterium, —C₀₋₄ alkylene-OR^{5a}, —C₀₋₄ alkylene-SR^{5a}, —C₀₋₄ alkylene-NR^{a}R^{5b};
R^{5a} and R^{5b} are each independently selected from hydrogen, deuterium, C₁₋₆ alkyl optionally substituted by halogen, C₂₋₆ alkenyl optionally substituted by halogen, C₂₋₆ alkynyl optionally substituted by halogen, C₁₋₆ alkyl optionally substituted by deuterium, C₂₋₆ alkenyl optionally substituted by deuterium, C₂₋₆ alkynyl optionally substituted by deuterium;
R^{x}, R⁶, R⁷, R⁸ each independently selected from hydrogen, halogen, cyano, C₁₋₆ alkyl optionally substituted by halogen, C₂₋₆ alkenyl optionally substituted by halogen, C₂₋₆ alkynyl optionally substituted by halogen, —OH, —O(optionally halogen-substituted-C₁₋₆ alkyl), —SH. —S(optionally halogen-substituted-C)₁₋₆ alkyl), —NH₂, —NH(optionally substituted)halogen substituted-C₁₋₆ alkyl), —N(optionally halogen-substituted-C₁₋₆ alkyl)₂; or
   R⁸ and R² together with the linking atoms form a saturated or unsaturated 4 to 10-membered heterocycloalkyl group;
Ring A is selected from saturated or unsaturated 3-10 membered carbocyclyl, saturated or unsaturated 4-10 membered heterocycloalkyl, 6-10 membered aromatic cyclyl, 5-10 membered aromatic heterocyclyl; wherein the carbocyclyl, heterocycloalkyl, aromatic ring, arylheterocyclyl may be further substituted with one, two, three, four or five independent R^{A1} substitution;
each R^{A1} is independently selected from halogen, cyano, C₁₋₆ alkyl optionally substituted by halogen, C₂₋₆ alkenyl optionally substituted by halogen, C₂₋₆ alkynyl optionally substituted by halogen, —C₀₋₄ alkylene-OR^{A2}, —C₀₋₄ alkylene-OC(O)R^{A2}, —C₀₋₄ alkylene-SR^{A2}, —C₀₋₄ alkylene-S(O)₂R^{A2}, —C₀₋₄ alkylene-S(O)R^{A2}, —C₀₋₄ alkylene-S(O)₂NR^{A2}R^{A3}, —C₀₋₄ alkylene-S(O)NR^{A2}R^{A3}, —C₀₋₄ alkylene-C(O)R^{A2}, —C₀₋₄ alkylene-C(O)OR^{A2}, —C₀₋₄ alkylene-C(O)NR^{A2}R^{A3}, —C₀₋₄ alkylene-NR^{A2}R^{A3}, —C₀₋₄ alkylene-NR^{A2}C(O)R^{A3}, —C₀₋₄ alkylene-NR^{A2}S(O)₂R^{A3}, —C₀₋₄ alkylene-NR^{A2}S(O)R^{A3}, —C₀₋₄ alkylene-(saturated or unsaturated 3 to 10-membered carbocyclic group), —C₀₋₄ alkylene-(saturated or unsaturated 4 to 10-membered heterocycloalkyl), —C₀₋₄ alkylene-(6 to 10-membered aromatic ring group), —C₀₋₄ alkylene-(5 to 10-membered aromatic heterocyclic group); wherein the alkylene, carbocyclyl, heterocycloalkyl, aromatic ring, aromatic heterocyclic group may be further substituted by one, two or three independent R^{A4} substitution; or
two independent R^{A1} together with the linking atom form

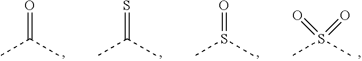

saturated or unsaturated 5 to 8-membered carbocyclyl, saturated or unsaturated 5- to 8-membered heterocycloalkyl, 5 to 8-membered aromatic cyclyl, 5 to 8-membered aromatic heterocyclyl;
R^{A2} and R^{A3} are each independently selected from hydrogen, C₁₋₆ alkyl optionally substituted by halogen, C₂₋₆ alkenyl optionally substituted by halogen, C₂₋₆ alkynyl optionally substituted by halogen, —C₀₋₄ alkylene-(saturated or unsaturated 3 to 10-membered carbocyclic group), —C₀₋₄ alkylene-(saturated or unsaturated 4- to 10-membered heterocycloalkyl), —C₀₋₄ alkylene-(6 to 10-membered aromatic ring group), —C₀₋₄ alkylene-(5 to 10-membered aromatic heterocyclic group); wherein the alkyl, alkenyl, alkynyl, alkylene, carbocyclyl, heterocycloalkyl, arylcyclyl, arylheterocyclyl may be further substituted with one, two, three, four or five independent R^{A4} substitution;
each R^{A4} is independently selected from halogen, cyano, C₁₋₆ alkyl optionally substituted by halogen, C₂₋₆ alkenyl optionally substituted by halogen, C₂₋₆ alkynyl optionally substituted by halogen, —C₀₋₄ alkylene-OR^{A5}, —C₀₋₄ alkylene-OC(O)R^{A5}, —C₀₋₄ alkylene-SR^{A5}, —C₀₋₄ alkylene-S(O)₂R^{A}, —C₀₋₄ alkylene-S(O)R^{A1}, —C₀₋₄ alkylene-S(O)₂NR^{A1}R^{A6}, —C₀₋₄ alkylene-S(O)NR^{A1}R^{A6}, —C₀₋₄ alkylene-C(O)R^{A1}, —C₀₋₄ alkylene-C(O)OR^{A1}, —C₀₋₄ alkylene-C(O)NR^{A5}R^{A6}, —C₀₋₄ alkylene-NR^{A5}R^{A6}, —C₀₋₄ alkylene-NR^{A5}C(O)R^{A6}, —C₀₋₄ alkylene-NR^{A5}S(O)₂R^{A6}, —C₀₋₄ alkylene-NR^{A5}S(O)R^{A6}; or two independent R^{A}4 together with the linking atom form

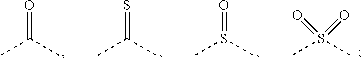

R^{A5} and R^{A6} are each independently selected from hydrogen, C₁₋₆ alkyl optionally substituted by halogen, C₂₋₆ alkenyl optionally substituted by halogen, C₂₋₆ alkynyl optionally substituted by halogen;
the heteroatoms in the saturated or unsaturated heterocyclic alkyl and the aromatic heterocyclic group are respectively and independently selected from one or more of 0, S, B or N; the unsaturated carbocyclic group does not include an aryl group, the unsaturated heterocycloalkyl group does not include an aromatic heterocyclic group;
or one or more of the variables are as defined in CN 115197196, the entirety of which is herein incorporated by reference.

In some embodiments, the present invention provides a compound of formula II-p:

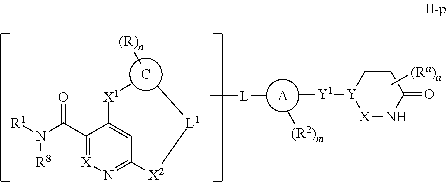

or a pharmaceutically acceptable salt thereof, wherein each of the variables in

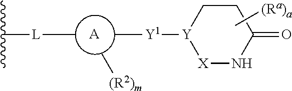

is as defined in the formulae above (e.g., formula I-a) and described in embodiments herein, both singly and in combination, and wherein:
X is selected from CH, N;
R¹ is selected from hydrogen, C₁₋₆ alkyl, deuterated C₁₋₆ alkyl, cycloalkyl;
R⁸ is selected from hydrogen, C₁₋₆ alkyl, C₁₋₆ alkoxy; or
R¹ is connected to R⁸, and forms a heterocycle together with the nitrogen atom to which it is connected;
X¹ and X² are independently selected from CH₂, NH, and O;
Ring C is selected from aryl, heteroaryl, cycloalkyl, heterocyclyl;
R is independently selected from R² or R³;
n is selected from 0, 1, 2, 3;
R² is selected from hydrogen, —OH, C₁₋₆ alkyl, deuterated C₁₋₆ alkyl, C₁₋₆ alkoxy, deuterated C₁₋₆ alkoxy, C₁₋₆ alkyl-C(O)—, C₁₋₆ alkyl-S—, C₁₋₆ alkyl-S(O)—, C₁₋₆ alkyl-S(O)₂;
R³ is selected from hydrogen, C₁₋₆ alkoxy-C(O)—, (R⁵)(R⁶)NC(O)—, or the following groups optionally substituted with one or more R^{a}: phenyl, heteroaryl, heterocyclyl, cycloalkyl;
R^{a} is selected from hydrogen, halogen, —CN, carboxyl, (R⁵)(R⁶)NC(O)—, C₁₋₆ alkyl, C₁₋₆ alkyl-C(O)—, C₁₋₆ alkyl-C(O)—NH—, halogenated C₁—, alkyl, halogenated C₁₋₆ alkoxy, hydroxy C₁₋₆ alkyl, C₁₋₆ alkoxy, cycloalkyl, cycloalkyl-(CH₂)ₚO—, NC-cycloalkyl, or any two adjacent R^{a} are connected to form a heterocycle together with the atoms to which they are connected;
p is selected from 0, 1, 2, 3;
L¹ is selected from C₁₋₈ alkylene optionally substituted by one or more R^{b}, and one or more CH₂ in said C₁₋₈ alkylene is optionally substituted by —C(O)—, —NR—, —S—, —S(O)—, —S(O)₂—, and/or —O-substituted;
R^{b} is selected from C₁₋₆ alkyl, or two R^{b} located on the same carbon atom and the carbon atom to which it is attached together form cycloalkyl, heterocyclyl;
alternatively, L¹ is selected from

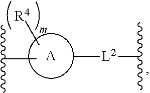

wherein the Ring A end is connected with X² and L end is connected with the Ring C;
Ring A is selected from aryl, heteroaryl;
L² is selected from C₁₋₆ alkylene optionally substituted by one or more R^{c}, and one or more CH₂ in said C₁₋₆ alkylene is optionally substituted by —C(O)—, —NR⁷—, —S—, —S(O)—, —S(O)₂— and/or —O-substituted;
R^{c} is selected from C₁₋₆ alkyl, or both are located on the same carbon atom R^{c} and its attached carbon atoms together form cycloalkyl, heterocyclyl;
R⁴ is selected from hydrogen, halogen, C₁₋₆ alkyl, halogenated C₁₋₆ alkyl, phenyl, cycloalkyl, and the phenyl is optionally composed of one or more selected from halogen, C₁₋₆ alkyl, C₁₋₆ alkoxy or halogenated C₁₋₆ alkyl group substituted;
m is selected from 0, 1, 2, 3;
R⁵ and R⁶ are independently selected from hydrogen, —OH, C₁₋₆ alkyl, cycloalkyl, or R⁵ and R⁶ are connected together, and together with the nitrogen atom to which they are connected, form a heterocycle;
R⁷ is selected from hydrogen, C₁₋₆ alkyl, C₁₋₆ alkoxy, C₁₋₆ alkyl-C(O)—, —C(O)OCH₂Ph; or one or more of the variables are as defined in WO 2022/213980, the entirety of which is herein incorporated by reference.

In some embodiments, the present invention provides a compound of formula II-q:

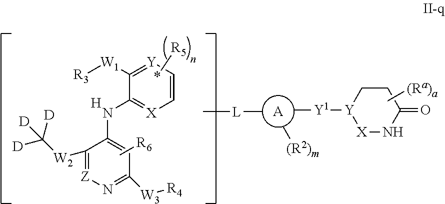

or a pharmaceutically acceptable salt thereof, wherein each of the variables in

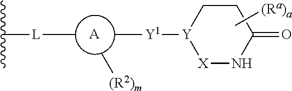

is as defined in the formulae above (e.g., formula I-a) and described in embodiments herein, both singly and in combination, and wherein:
X and Y are each independently CR₅ or N;
Z is CR₆ or N;
W₁ and W₂ are each independently a divalent group selected from the group consisting of O, S, SO₂, CO, NR₇CO, CONR₇;
W₃ is a divalent group selected from the group consisting of CO, NR₇, NR₇CO, CONR₇;
R₃ is substituted or unsubstituted C₃₋₆ cycloalkyl;
R₄ is selected from the group consisting of substituted or unsubstituted C₁₋₆ alkyl, substituted or unsubstituted C₃₋₆ cycloalkyl; and R₄ is also optionally substituted with R₁ and R₂;
R₁ and R₂ are each independently selected from the group consisting of H, substituted or unsubstituted C₁₋₆ alkyl; alternatively, R₁ and R₂ and the carbon atom to which they are attached together form a substituted or unsubstituted C₃₋₆ cycloalkyl;
R₅, R₆ and R₇ are each independently selected from the group consisting of H, substituted or unsubstituted C₁₋₄ alkyl;
n is 1 or 2;
or one or more of the variables are as defined in WO 2022/206705, the entirety of which is herein incorporated by reference.

In some embodiments, the present invention provides a compound of formula II-r-1 or II-r-2:

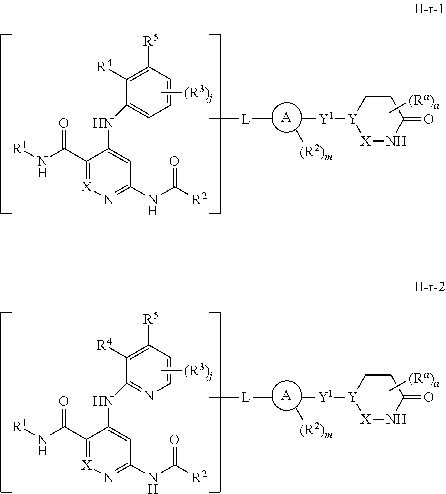

or a pharmaceutically acceptable salt thereof wherein each of the variables in

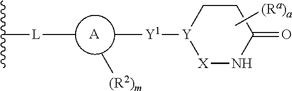

is as defined in the formulae above (e.g., formula I-a) and described in embodiments herein, both singly and in combination, and wherein:
X is CH or N;
R¹ is C₁₋₃ alkyl substituted by 0-7 deuterium atoms;
R² is optionally substituted C₁₋₆ alkyl, optionally substituted C₁₋₄ heteroalkyl, optionally substituted cycloalkyl (e.g., C₃₋₆ cycloalkyl), optionally substituted heterocyclyl (e.g., 4-8 membered heterocyclyl), or optionally substituted heteroaryl;
R³ at each occurrence is independently halogen, optionally substituted C₁₋₄ alkyl, or optionally substituted C₁₋₄ heteroalkyl;
j is 0, 1, 2, or 3;
R⁴ is C₁₋₆ alkyl optionally substituted with 1-3 R^{A}, S(O)ₚR^{B}, or OR¹;
p is 0, 1, or 2,
R^{A} at each occurrence is independently halogen, OH, C₁₋₆ alkyl optionally substituted with 1-3 R^{A1},
R^{B} is C₁₋₆ alkyl optionally substituted with 1-3 R^{A1},
R^{C} is hydrogen or C₁₋₆ alkyl optionally substituted with 1-3 R^{A2}
R^{A1} at each occurrence is independently halogen, OH, or CN;
R^{A2} at each occurrence is independently F or OH;
R⁵ is an optionally substituted heterocyclyl or an optionally substituted heteroaryl such as

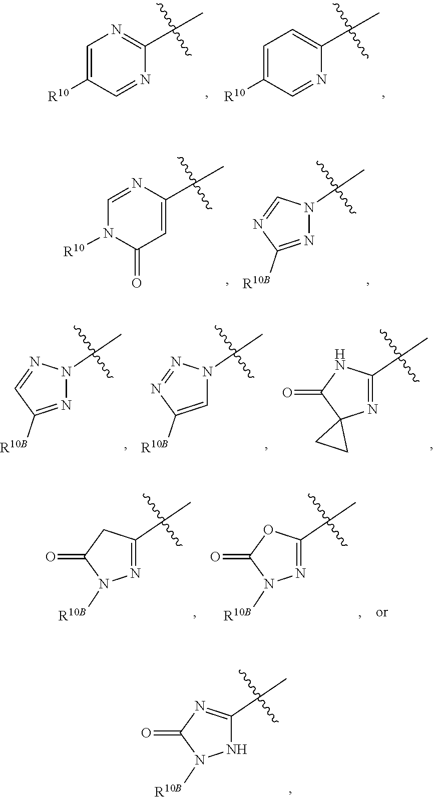

or R⁵ is -L¹-L²-Q-G;
R¹⁰ at each occurrence is independently an optionally substituted cycloalkyl (e.g., C₃₋₆ cycloalkyl), or optionally substituted heterocyclyl (e.g., 4-8 membered heterocyclyl);
R^{10B} at each occurrence is independently halogen, CN, an optionally substituted C₁₋₆ alkyl, an optionally substituted cycloalkyl (e.g., C₃₋₆ cycloalkyl), or optionally substituted heterocyclyl (e.g., 4-8 membered heterocyclyl);
L¹ is O, C(O), NH, or null;
L² is C₁₋₄ alkylene, or null;
Q is an optionally substituted heterocycle or optionally substituted heteroaryl, and
G is CN,

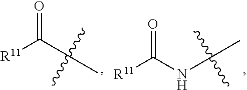

or a Michael acceptor;
R¹¹ is hydrogen, an optionally substituted C₁₋₆ alkyl or an optionally substituted C₃₋₆ cycloalkyl; or one or more of the variables are as defined in WO 2022/193499, the entirety of which is herein incorporated by reference.

In some embodiments, the present invention provides a compound of formula II-s:

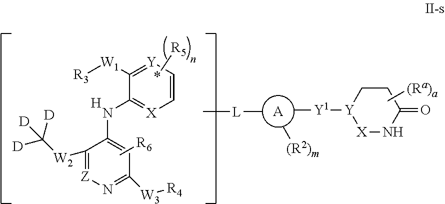

or a pharmaceutically acceptable salt thereof, wherein each of the variables in

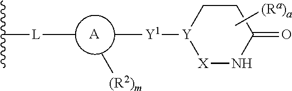

is as defined in the formulae above (e.g., formula I-a) and described in embodiments herein, both singly and in combination, and wherein:
X and Y are each independently CR₅ or N;
Z is CR₆ or N;
W₁ and W₂ are each independently a divalent group selected from the group consisting of O, S, SO₂, CO, NR₇CO, CONR₇;
W₃ is a divalent group selected from the group consisting of CO, NR₇, NR₇CO, CONR₇;
R₃ is substituted or unsubstituted C₃₋₆ cycloalkyl;
R₄ is selected from the group consisting of substituted or unsubstituted C₁₋₆ alkyl, substituted or unsubstituted C₃₋₆ cycloalkyl; and R₄ is also optionally substituted with R₁ and R₂;
R₁ and R₂ are each independently selected from the group consisting of H, substituted or unsubstituted C₁₋₆ alkyl; alternatively- R₁ and R₂ and the carbon atom to which they are attached together form a substituted or unsubstituted C₃₋₆ cycloalkyl;
R₅, R₆ and R₇ are each independently selected from the group consisting of H, substituted or unsubstituted C₁₋₄ alkyl;
n is 1 or 2;
or one or more of the variables are as defined in WO 2022/206705, the entirety of which is herein incorporated by reference.

In some embodiments, the present invention provides a compound of formula II-t:

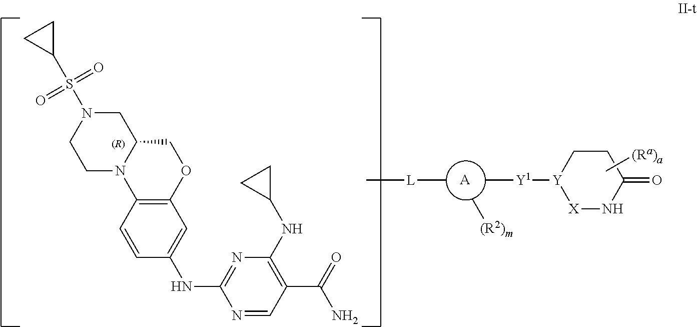

or a pharmaceutically acceptable salt thereof, wherein each of the variables is as defined in the formulae above (e.g., formula I-a) and described in embodiments herein, both singly and in combination.

In some embodiments, the present invention provides a compound of formula II-u:

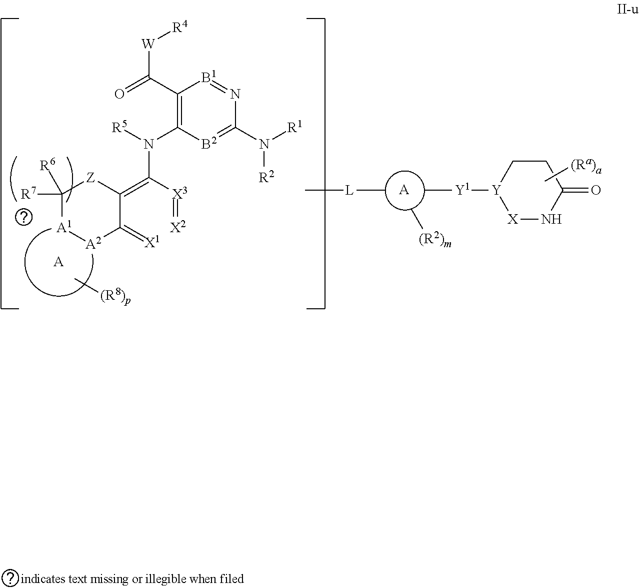

or a pharmaceutically acceptable salt thereof wherein each of the variables in

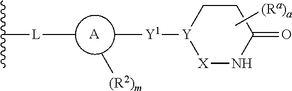

is as defined in the formulae above (e.g., formula I-a) and described in embodiments herein, both singly and in combination, and wherein:
Ring A is an unsubstituted or substituted 5-membered heterocyclic ring wherein A¹ and A² are independently N or C, wherein if Ring A is substituted then Ring A is substituted with p instances of R⁸;
each R⁸ is independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted carbocycle, unsubstituted or substituted heterocycle, —CN, —OH, —OR¹⁷, —C(═O)R¹⁶, —CO₂R¹⁶, —C(═O)N(R¹⁶)₂, —N(R¹⁶)₂, —NR¹⁶C(═O)R¹⁷, —SR¹⁶, —S(═O)R¹⁷, —SO₂R¹⁷, or —SO₂N(R¹⁶)₂; wherein if R⁸ is attached to a nitrogen atom, then R⁸ is hydrogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted carbocycle, unsubstituted or substituted heterocycle, —C(═O)R¹⁶, —CO₂R¹⁶, —C(═O)N(R¹⁶)₂, —S(═O)R¹⁷, —SO₂R¹⁷, or —SO₂N(R¹⁶)₂; or
two R⁸ attached to the same carbon atom are taken together to form ═O, ═S, or ═NH;
Z is —NR¹⁰—, —O—, —S—, —S(═O)—, or —SO₂—;
R¹⁰ is hydrogen, C₁-C₆ alkyl, C₁-C₆ deuteroalkyl, C₁-C₆ fluoroalkyl, C₃-C₆ cycloalkyl, or monocyclic heterocycle;
X¹, X², and X³ are each independently CR¹¹ or N;
each R¹¹ is independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted carbocycle, unsubstituted or substituted heterocycle, —CN, —OH, —OR⁷, —C(═O)R¹⁶, —CO₂R¹⁶, —C(═O)N(R⁶)₂, —N(R¹⁶)₂, —NR¹⁶C(═O)R¹⁷, —SR¹⁶, —S(═O)R¹⁷, —SO₂R¹⁷, or —SO₂N(R¹⁶)₂;
B¹ is N or CR^{12a};
B² is N or CR^{12b};
R^{12a} and R^{12b} are each independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted carbocycle, unsubstituted or substituted heterocycle, —CN, —OH, —OR¹⁷, —C(═O)R¹⁶, —CO₂R¹⁶, —C(═O)N(R¹⁶)₂, —N(R¹⁶)₂, —NR¹⁶C(═O)R⁷, —SR¹⁶, —S(═O)R¹⁷, —SO₂R¹⁷, or —SO₂N(R¹⁶);
R¹ is hydrogen, C₁-C₆ alkyl, or C₁-C₆ fluoroalkyl;
R² is a Ring B that is an unsubstituted or substituted heterocycle or unsubstituted or substituted carbocycle, wherein if Ring B is substituted then Ring B is substituted with q instances of R¹³;
each R¹³ is independently halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted carbocycle, unsubstituted or substituted heterocycle, —CN, —OH, —OR¹⁷, —C(═O)R¹⁶, —CO₂R¹⁶, —C(═O)N(R¹⁶)₂, —N(R¹⁶)₂, —NR¹⁶C(═O)R¹⁷, —SR¹⁶, —S(═O)R¹⁷, —SO₂R¹⁷, or —SO₂N(R¹⁶)₂; or
   two R¹³ groups on adjacent atoms of Ring B are taken together with the intervening atoms to which they are attached to form an unsubstituted or substituted 5- or 6-membered monocyclic carbocycle or an unsubstituted or substituted 5- or 6-membered monocyclic heterocycle; or
   R² is —C(═O)R¹⁴, —C(═O)NR¹⁴R¹⁵, or —C(═O)OR¹⁴;
R¹⁴ is hydrogen, unsubstituted or substituted C₁-C₆ alkyl, C₁-C₆ deuteroalkyl, unsubstituted or substituted C₂-C₆ alkenyl, unsubstituted or substituted C₂-C₆ alkynyl, unsubstituted or substituted C₁-C₆ heteroalkyl, unsubstituted or substituted monocyclic carbocycle, unsubstituted or substituted bicyclic carbocycle, unsubstituted or substituted monocyclic heterocycle, or unsubstituted or substituted bicyclic heterocycle;
R¹⁵ is hydrogen, C₁-C₆ alkyl, or C₁-C₆ fluoroalkyl; or
   R¹⁴ and R¹⁵ are taken together with the intervening atoms to which they are attached to form an unsubstituted or substituted 4- to 6-membered monocyclic heterocycle; or
   R¹ and R¹⁵ are taken together with the intervening atoms to which they are attached to form an unsubstituted or substituted 5- or 6-membered monocyclic heterocycle;
W is —NR³— or —O—;
R³ is hydrogen, C₁-C₆ alkyl, C₁-C₆ deuteroalkyl, C₁-C₆ fluoroalkyl, C₃-C₆ cycloalkyl, or monocyclic heterocycle;
R⁴ is hydrogen, C₁-C₆ alkyl, C₁-C₆ deuteroalkyl, C₁-C₆ fluoroalkyl, C₃-C₆ cycloalkyl, or monocyclic heterocycle; or
   R³ and R⁴ are taken together with the N atom to which they are attached to form a substituted or unsubstituted N-containing heterocycle; or
   R³ and R^{12a} are taken together with the intervening atoms to which they are attached to form a substituted or unsubstituted 5- or 6-membered heterocycle;
R⁵ is hydrogen, C₁-C₆ alkyl, C₁-C₆ fluoroalkyl, C₃-C₆ cycloalkyl, or monocyclic heterocycle;
each R⁶ and R⁷ is independently hydrogen, deuterium, halogen, C₁-C₆ alkyl, C₁-C₆ deuteroalkyl, C₁-C₆ fluoroalkyl, C₃-C₆ cycloalkyl, or monocyclic heterocycle, —CN, —OH, —OR¹⁷, —C(═O)R¹⁶, —CO₂R¹⁶, —C(═O)N(R¹⁶)₂, —N(R¹⁶)₂, —NR¹⁶C(═O)R¹⁷, —SR¹⁶, —S(═O)R¹⁷, —SO₂R¹⁷, or —SO₂N(R¹⁶)₂; or one R⁶ and one R⁷ attached to the same carbon atom are taken together with the carbon atom to which they are attached to form C═O or C₃-C₄ cycloalkyl;
each R¹⁶ is independently hydrogen, substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₇ cycloalkyl, substituted or unsubstituted monocyclic 3- to 8-membered heterocycloalkyl, substituted or unsubstituted phenyl, or substituted or unsubstituted monocyclic heteroaryl; or
two R¹⁶ on the same N atom are taken together with the N atom to which they are attached to form a substituted or unsubstituted N-containing heterocycle; and
each R¹⁷ is independently substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₇ cycloalkyl, substituted or unsubstituted monocyclic 3- to 8-membered heterocycloalkyl, substituted or unsubstituted phenyl, or substituted or unsubstituted monocyclic heteroaryl; wherein each substituted alkyl, substituted fluoroalkyl, substituted deuteroalkyl, substituted alkoxy, substituted fluoroalkoxy, substituted heteroalkyl, substituted carbocycle, and substituted heterocycle is substituted with one or more R⁵ groups independently selected from the group consisting of deuterium, halogen, C₁-C₆ alkyl, monocyclic carbocycle, monocyclic heterocycle, —CN, —CH₂CN, —OR¹⁸, —CH₂OR¹⁸, —CO₂R¹⁸, —CH₂CO₂R¹⁸, —C(═O)N(R¹⁸)₂, —CH₂C(═O)N(R¹⁸)₂, —N(R¹⁸)₂, —CH₂N(R¹⁸)₂, —NR¹⁸C(═O)R¹⁸, —CH₂NR¹⁸C(═O)R¹⁸, —NR⁸SO₂R¹⁹, —CH₂NR¹⁸SO₂R¹⁹, —SR¹⁸, —CH₂SR¹⁸, —S(═O)R¹⁹, —CH₂S(═O)R¹⁹, —SO₂R¹⁹, —CH₂SO₂R¹⁹, —SO₂N(R¹⁸)₂, or —CH₂SO₂N(R¹⁸)₂; each R¹⁸ is independently selected from hydrogen, C₁-C₆ alkyl, C₁-C₆ fluoroalkyl, C₁-C₆ heteroalkyl, C₃-C₆ cycloalkyl, C₂-C₆ heterocycloalkyl, phenyl, benzyl, 5-membered heteroaryl and 6-membered heteroaryl; or
   two R¹⁸ groups are taken together with the N atom to which they are attached to form a N-containing heterocycle;
each R¹⁹ is independently selected from C₁-C₆ alkyl, C₁-C₆ heteroalkyl. C₃-C₆ cycloalkyl, C₂-C₆ heterocycloalkyl, phenyl, benzyl, 5-membered heteroaryl, and 6-membered heteroaryl;
n is 1, 2, or 3;
p is 0, 1, 2, or 3; and
q is 0, 1, 2, 3, or 4;
or one or more of the variables are as defined in WO 2022/175745, WO 2022/175746, WO 2022/175747, and WO 2022/175752, the entirety of which is herein incorporated by reference.

In some embodiments, the present invention provides a compound of formula II-v:

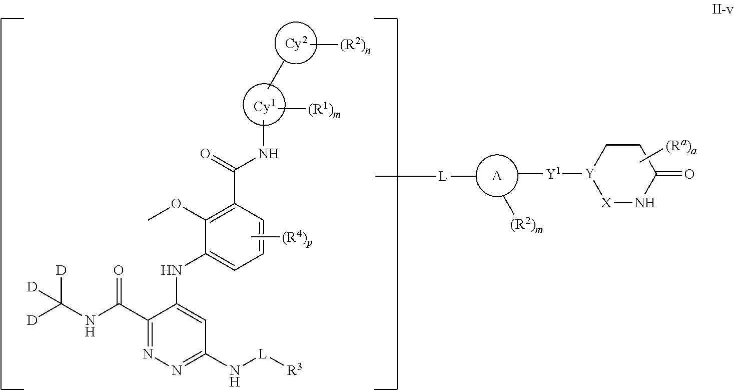

or a pharmaceutically acceptable salt thereof wherein each of the variables in

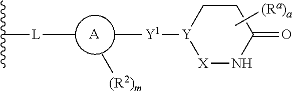

is as defined in the formulae above (e.g., formula I-a) and described in embodiments herein, both singly and in combination, and wherein:
Cy¹ and Cy² are each independently selected from C₆₋₁₀ aryl or 5-10 membered heteroaryl;
each R¹ or R² are independently selected from halogen, hydroxy, amino, cyano, C₁₋₈ alkyl, C₃₋₁₀ cycloalkyl or 5 to 10-membered heterocycloalkyl, said C₁₋₈ alkyl, C₃₋₁₀ cycloalkyl or 5-10 membered heterocycloalkyl optionally substituted with one or more halogen, deuterium, hydroxy or cyano;
m is selected from 0, 1, 2, 3, 4, or 5;
n is selected from 0, 1, 2, 3, 4, or 5;
L is selected from —C(O)— or a bond;
R³ is selected from 5-10 membered heteroaryl, C₃₋₁₀ cycloalkyl, C₆₋₁₀ aryl or 3-10 membered heterocycloalkyl, wherein said 5-10 membered heteroaryl, C₃₋₁₀ cycloalkyl, C₆₋₁₀ aryl or 3-10 membered heterocycloalkyl optionally substituted with one or more R^{a} substitution;
each R⁴ Each independently selected from halogen;
p is selected from 0, 1, 2, or 3;
R^{a} selected from halogen, cyano, C₁₋₈ alkyl, C₁₋₈ alkoxy, C₃₋₁₀ cycloalkyloxy, C₃₋₁₀ cycloalkyl, 5-10 membered heteroaryl, 3-10 membered heterocycloalkyl, amino. C₁₋₈ alkylNH—, (C₁₋₈ alkyl)₂N—, C₃₋₁₀ cycloalkylNH— or C₁₋₈ alkyl substituted by one or more halogens;
or one or more of the variables are as defined in CN 114907326, the entirety of which is herein incorporated by reference.

In some embodiments, the present invention provides a compound of formula II-w:

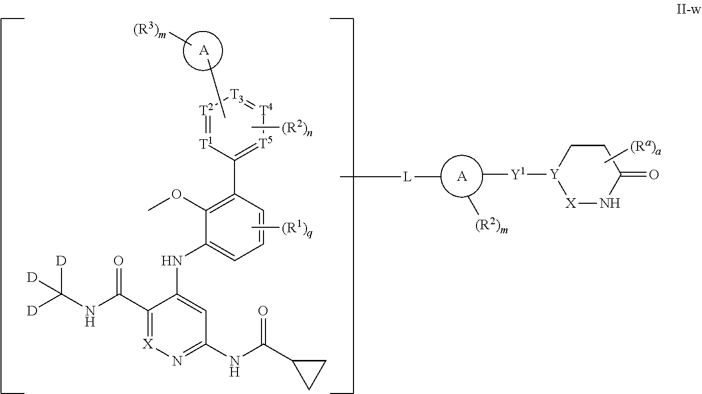

or a pharmaceutically acceptable salt thereof, wherein each of the variables in

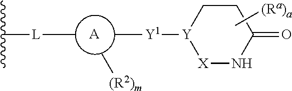

is as defined in the formulae above (e.g., formula I-a) and described in embodiments herein, both singly and in combination, and wherein:
X is selected from N or CH;
each R¹ is independently selected from halogen;
q is selected from 0, 1, or 2;
each R is independently selected from halogen, hydroxy, amino, cyano, or nitro;
n is selected from 0, 1, or 2;
T¹, T², T³, T⁴, or T⁵ are each independently selected from CH or N, at least one of which is selected from CH;
Ring A is selected from C₃₋₁₀ cycloalkyl, 3-10 membered heterocyclyl, C₆₋₁₀ aryl or 5-10 membered heteroaryl;
each R³ is independently selected from ═O, halogen, hydroxy, amino, cyano, nitro, C₁₋₈ alkyl or C₁₋₈ alkoxy, said C₁₋₈ alkyl or C₁₋₈ alkoxy is optionally substituted with one or more R^{a};
m is selected from 0, 1, 2, 3, or 4;
each R^{a} is independently selected from halogen, hydroxy, amino, or cyano;
or one or more of the variables are as defined in WO 2022/166917, the entirety of which is herein incorporated by reference.

In some embodiments the present invention provides a compound of formula II-x:

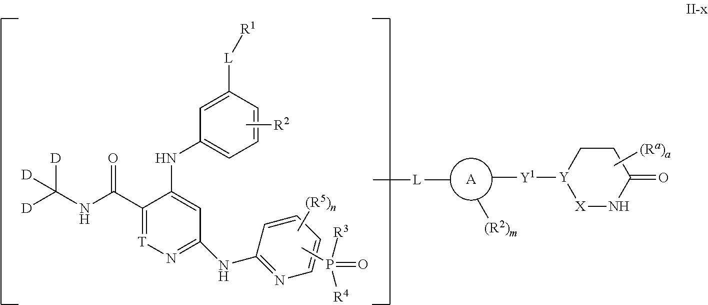

or a pharmaceutically acceptable salt thereof, wherein each of the variables in

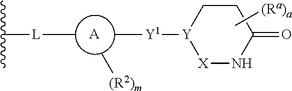

is as defined in the formulae above (e.g., formula I-a) and described in embodiments herein, both singly and in combination, and wherein:
-L-R¹ is selected from —CONH—R¹, —NHCO—R¹, —CO—R¹, —CONHNH—R¹, —NHNHCO—R¹ or —R¹;
R¹ is selected from 5-10 membered heteroaryl, C₂₋₆ alkynyl, cyano, C₁₋₆ alkyl, hydrogen, C₃₋₁₀ cycloalkyl, C₆₋₁₀ aryl or 3-10 membered heterocycloalkyl wherein said 5-10 membered heteroaryl, C₆₋₁₀ aryl, C₃₋₁₀ cycloalkyl or 3-10 membered heterocycloalkyl optionally substituted with one or more R^{a} substitution;
R^{a} selected from halogen, carboxyl, cyano, aldehyde group, C₁₋₆ alkyl, C₁₋₆ alkoxy, C₃₋₁₀ cycloalkyl, 5-10 membered heteroaryl, 3-10 membered heterocycloalkyl, —CONH₂ or —CO₂C₁₋₄ alkyl, wherein said C₁₋₆ alkyl or C₁₋₆ alkoxy is optionally substituted with one or more groups selected from hydroxy and halogen, wherein said C₃₋₁₀ cycloalkyl, 5-10 membered heteroaryl or 3-10 membered heterocycloalkyl is optionally substituted with one or more groups selected from C₁₋₆ alkyl, hydroxyl, and halogen, wherein the C₁₋₆ alkyl is optionally substituted by one or more hydroxy or halogen groups;
R² is selected from C₁₋₆ alkoxy, wherein said C₁₋₆ alkoxy is optionally substituted with one or more groups selected from halogen or hydroxyl;
n is 0, 1, 2, or 3;
R³ and R⁴ are each independently selected from a C₁₋₆ alkyl group;
each R⁵ is independently selected from halogen, C₁₋₆ alkyl, C₁₋₆ alkyl-O—, C₁₋₆ alkyl-S—, C₁₋₆ alkyl-NH—, C₃₋₁₀ cycloalkyl-NH—, C₆₋₁₀ aryl-NH—, 5-10 membered heteroaryl-NH—, C₃₋₁₀ cycloalkyl-CONH—, C₆₋₁₀ aryl or 5-10 membered heteroaryl, wherein said C₁₋₆ alkyl radical, C₃₋₁₀ cycloalkyl-CONH—, C₆₋₁₀ aryl-NH— or 5-10 membered heteroaryl-NH—, C₆₋₁₀ aryl or 5-10 membered heteroaryl optionally substituted with one or more groups selected from halogen, hydroxy or cyano;
t is selected from CH or N;
or one or more of the variables are as defined in CN 114805438, the entirety of which is herein incorporated by reference.

In some embodiments, the present invention provides a compound of any one of the following formulae:

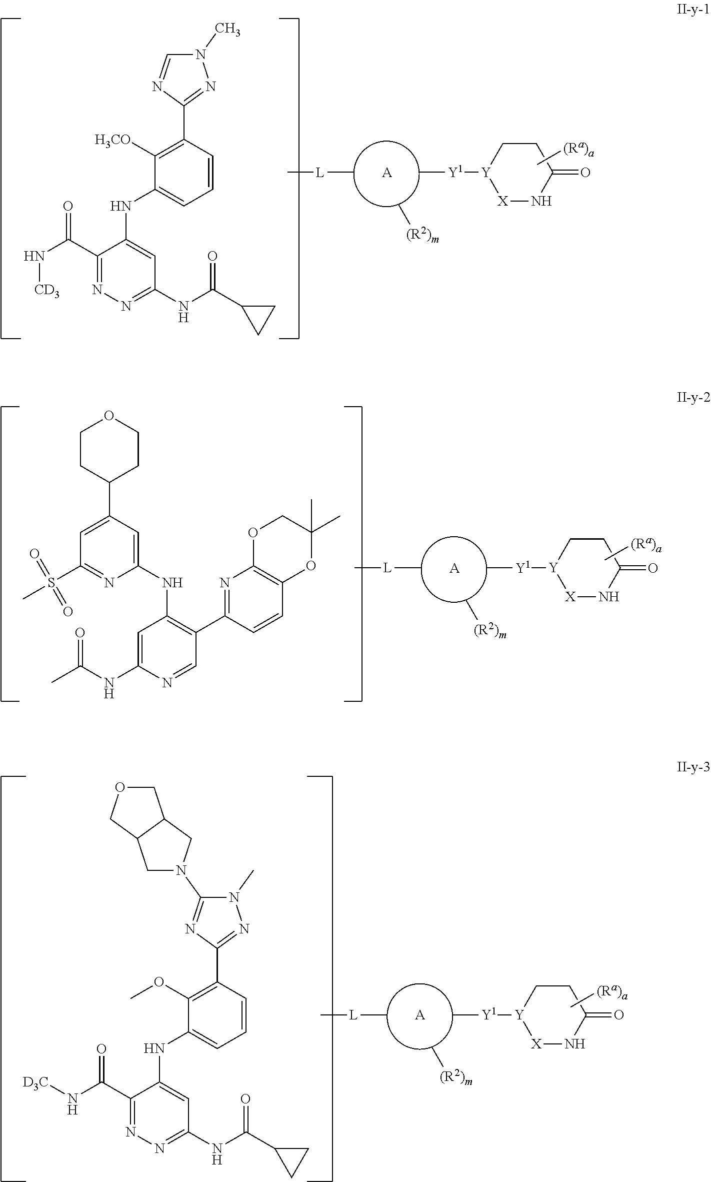

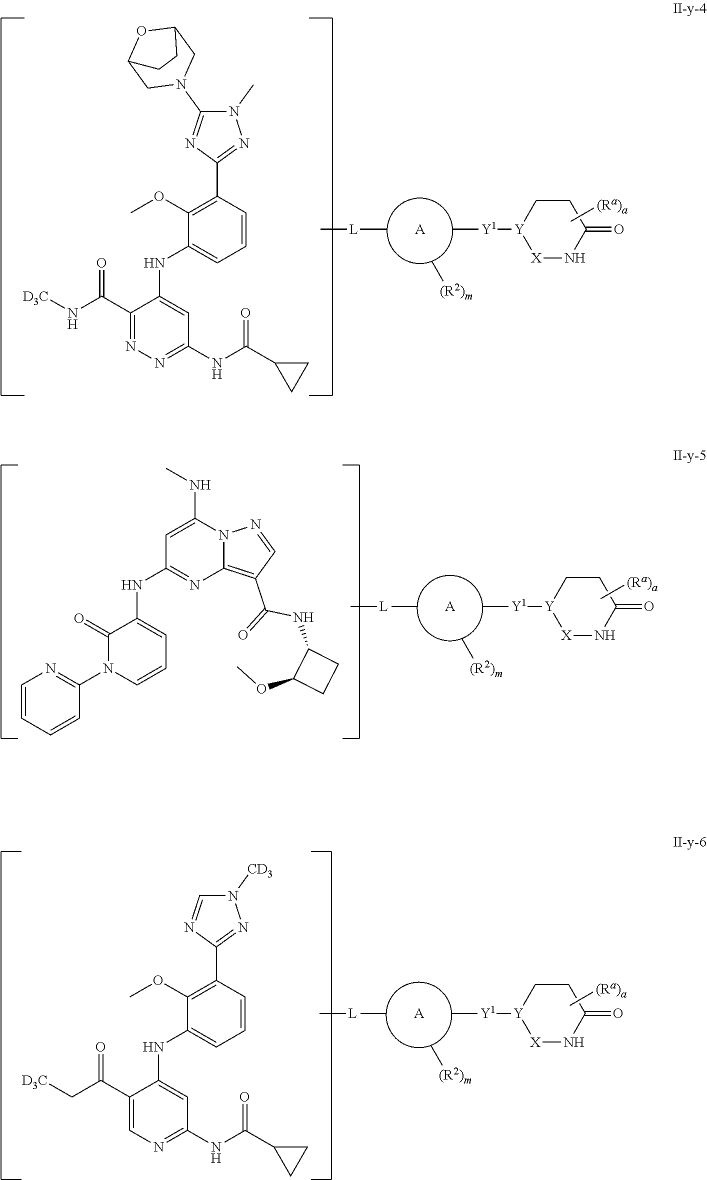

or a pharmaceutically acceptable salt thereof, wherein each of the variables is as defined in the formulae above (e.g., formula I-a) and described in embodiments herein, both singly and in combination.

In some embodiments, the present invention provides a compound of formula II-z-1 or II-z-2:

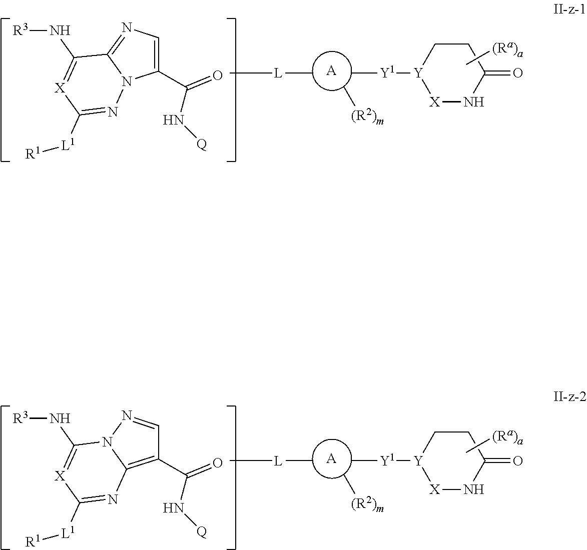

or a pharmaceutically acceptable salt thereof, wherein each of the variables in

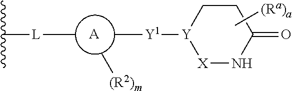

is as defined in the formulae above (e.g., formula I-a) and described in embodiments herein, both singly and in combination, and wherein:
L¹ is a bond, NR¹⁰, O, optionally substituted C₁₋₄ alkyelene or optionally substituted C₁₋₄ heteroalkyelene;
R¹ is an optionally substituted carbocyclic ring, optionally substituted heterocyclic ring, optionally substituted aryl or optionally substituted heteroaryl ring;
X is N or CR², wherein R² is hydrogen, hydroxyl, halogen, optionally substituted C₁₋₆ alkyl, optionally substituted C₁₋₆ heteroalkyl, optionally substituted C₁₋₆ alkoxy, optionally substituted C₃₋₆ cycloalkyl, or optionally substituted 4-8 membered heterocyclyl;
R³ is hydrogen, optionally substituted C₁₋₆ alkyl, optionally substituted C₁₋₆ heteroalkyl, optionally substituted C₃₋₆ cycloalkyl, or optionally substituted 4-8 membered heterocyclyl; or R² and R³, together with the intervening atoms, form a 5-8 membered heterocyclic or a 5 or 6 membered heteroaryl ring, each of which is optionally substituted and has one ring nitrogen atom, and optionally 1-2 additional ring heteroatoms independently selected from nitrogen, oxygen, and sulfur;
Q is 6-14 membered heterocyclyl or 5-10 membered heteroaryl, each of which is optionally substituted, or
Q is

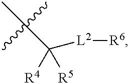

wherein R⁴ and R⁵ are each independently hydrogen or an optionally substituted C₁₋₆ alkyl, or R⁴ and R⁵ are joined to form a 3-8 membered carbocyclic or heterocyclic ring, each of which is optionally substituted;
L² is a bond, NR^{11A}, O, optionally substituted C₁₋₄ alkylene or optionally substituted C₁₋₄ heteroalkylene;
R⁶ is hydrogen, a 3-8 membered heterocyclic ring, or a 5 or 6 membered heteroaryl ring, each of which is optionally substituted; and wherein:
R¹⁰ and R^{11A} are each independently hydrogen, optionally substituted C₁₋₆ alkyl, optionally substituted C₃₋₆ cycloalkyl, optionally substituted aryl (e.g., phenyl), optionally substituted heteroaryl (e.g., 5 or 6 membered heteroaryl), or optionally substituted 4-8 membered heterocyclyl;
or one or more of the variables are as defined in WO 2022/156657, the entirety of which is herein incorporated by reference.

In some embodiments, the present invention provides a compound of formula II-aa:

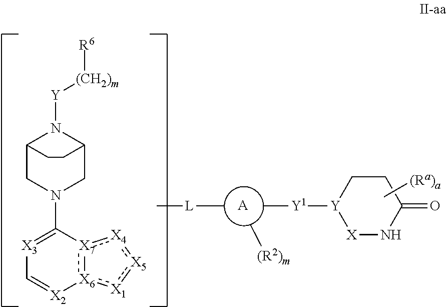

or a pharmaceutically acceptable salt thereof, wherein each of the variables in

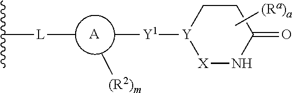

is as defined in the formulae above (e.g., formula I-a) and described in embodiments herein, both singly and in combination, and wherein:
[symbol] is a single bond or double bond, provided the ring containing X₁, X₂, X₃, X₄, X₅, X₆ and X₇ is a bicyclic heteroaryl ring;
X₁ is N, NH, or CR¹;
X₂ is N or CR²;
X₃ is N or CR³;
X₄ is N or CR⁴;
X₅ is NR⁵ or CRS;
X₆ and X₇ are both C, or one of X₆ and X₇ is N and the other is C;
Y is C(O) or S(O)₂;
R¹, R², R³ and R⁴, when present, are each independently selected from H, halo, —CN, —NR^{1a}R^{1b}, —OR^{1c}, C₁₋₄ alkyl and C₁₋₄ haloalkyl;
R⁵ is selected from H, halo, CN, —NR^{1a}R^{1b}, —OR^{1c}, C₁₋₆ alkyl, C₃₋₈ cycloalkyl, C₆₋₁₀ aryl, 4 to 10 membered heterocycloalkyl, 5 to 7 membered partially saturated heterocyclyl, and 5 to 10 membered heteroaryl, wherein the C₁₋₆ alkyl, C₃₋₈ cycloalkyl, C₁₋₁₀ aryl, 4 to 10 membered heterocycloalkyl and 5 to 10 membered heteroaryl represented by R⁵ are each optionally substituted with one or more R⁷;
R⁶ is H, C₁₋₆ alkyl, C₃₋₈ cycloalkyl, C₆₋₁₀ aryl, 4 to 10 membered heterocycloalkyl or 5 to 10 membered heteroaryl, wherein the C₁₋₆ alkyl, C₃₋₈ cycloalkyl, C₆₋₁₀ aryl, 4 to 10 membered heterocycloalkyl and 5 to 10 membered heteroaryl represented by R are each optionally substituted with one or more R⁸;
R⁷, for each occurrence, is independently selected from halo, —CN, oxo (═O), —NR^{1a}R^{1b}, —OR^{1c}, —C(O)OR^{1c}, C₁₋₆ alkyl, C₁₋₆ haloalkyl, C₃₋₆ cycloalkyl, C₆₋₁₀ aryl, 4 to 7 membered monocyclic heterocycloalkyl, and 5 to 6 membered heteroaryl; wherein the C₁₋₆ alkyl, C₁₋₆ haloalkyl, C₃₋₆ cycloalkyl, C₆₋₁₀ aryl, 4 to 7 membered monocyclic heterocycloalkyl, and 5 to 6 membered heteroaryl represented by R⁷ are each optionally substituted with one or more substituents independently selected from halo, C₁₋₄ alkyl, C₁₋₄ haloalkyl, C₃₋₆ cycloalkyl, —NR^{1a}R^{1b}, —OR^{1c} and 4 to 6 membered monocyclic heterocycloalkyl;
R⁸, for each occurrence, is independently selected form halo, —NR^{1a}R^{1b}, —OR^{1c}, —CN, C₁₋₆ alkyl, C₁₋₃ hydroxyalkyl, —C(═O)OR^{1c}, and C₁₋₆ haloalkyl R^{1a} and R^{1b} are each independently H or C₁₋₄ alkyl; R^{1c} is H, C₁₋₄ alkyl or C₁₋₄ haloalkyl; and
m is 0 or an integer from 1 to 6;
or one or more of the variables are as defined in WO 2022/150446, the entirety of which is herein incorporated by reference.

In some embodiments, the present invention provides a compound of formula II-bb:

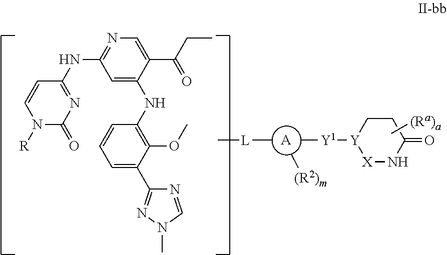

or a pharmaceutically acceptable salt thereof, wherein each of the variables in

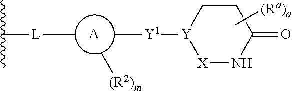

is as defined in the formulae above (e.g., formula I-a) and described in embodiments herein, both singly and in combination, and wherein:
R is methyl, phenyl substituted with at least one F atom, optionally substituted heteroaryl containing 1, 2, or 3 N atoms, —CH₂—R² or —CH₂CH₂—R³,
R² is cyclopropyl, cyclopropyl substituted with 1-3 substituents selected from F and methyl, cyclobutyl, cyclobutyl substituted with 1-3 substituents selected from F and methyl, oxetane or oxetane substituted with 1-3 substituents selected from F and methyl, tetrahydrofuran, or tetrahydrofuran substituted with 1-3 substituents selected from F and methyl; and
R³ is —OH, —OMe, -OEt, —OPr, -OiPr, —NH₂, —NHMe, —NMe₂;
or are as defined in WO 2023/064223, the entirety of which is herein incorporated by reference.

In some embodiments, the present invention provides a compound of formula II-cc:

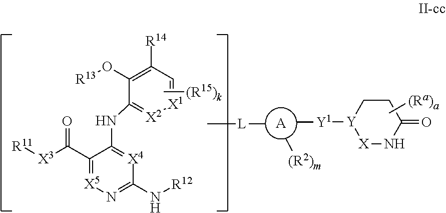

or a pharmaceutically acceptable salt thereof, wherein each of the variables in

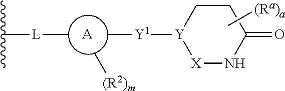

is as defined in the formulae above (e.g., formula I-a) and described in embodiments herein, both singly and in combination, and wherein:
each of X¹ and X² is independently selected from CH and N;
each of X⁴ and X⁵ is independently selected from CH, CF and N;
X³ is NR, O, CH₂, or CF₂;
R¹¹ is a H, F, C₁-C₃ alkyl, or CD₃;
R¹² is C(═O)R^{12′} or R^{12′}, wherein R^{12′} is a C₁-C₆ alkyl, C₃-C₆ cycloalkyl, aryl or heteroaryl optionally substituted with halogen, CF₃, CN, OR, amino, alkyl, cycloalkyl, heterocyclic, aryl or heteroaryl;
R¹³ is a C₁-C₃ alkyl, CD₃ or CF₃;
R¹⁴ is H or a 5 or 6-membered heteroaryl group comprising 1, 2 or 3 hetero atoms selected from N, O and S, or R¹⁴ is OR¹⁴, wherein R^{14′} is C₁-C₆ alkyl or heteroalkyl or a C₃-C₆ cycloalkyl or heterocycloalkyl;
R¹⁵ at each occurrence is independently selected from F, C₁, CN, OR, and a C₁-C₃ alkyl;
R at each occurrence is independently H or a C₁-C₆ alkyl; and
k is 0, 1, 2, or 3;
or are as defined in WO 2023/064223, the entirety of which is herein incorporated by reference.

In some embodiments, the present invention provides a compound of formula II-dd:

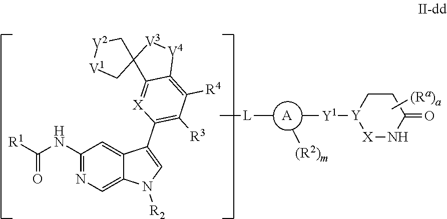

or a pharmaceutically acceptable salt thereof, wherein each of the variables in

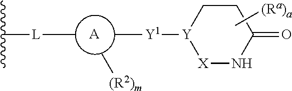

is as defined in the formulae above (e.g., formula I-a) and described in embodiments herein, both singly and in combination, and wherein:
X is N or CR^{x};
R¹ is —NH₂. C₁₋₆ alkyl, C₁₋₆ deuterated alkyl, C₁₋₆ haloalkyl, C₁₋₆ alkoxy, C₁₋₆ alkylamino, C₃₋₈ ring alkyl, heterocyclic group consisting of 3-8 atoms, C₆₋₁₀ aryl group or heteroaryl group consisting of 5-12 atoms, wherein the C₁₋₆ alkyl, C₁₋₆ alkoxy, C₁₋₆ alkylamino, C₃₋₈ cycloalkyl, heterocyclyl consisting of 3-8 atoms, C₆₋₁₀ aryl and heteroaryl consisting of 5-12 atoms are independently and optionally substituted by 1, 2, 3, 4, or 5 substituents selected from D, F, Cl, Br, I, —NO₂, —CN, —OH, —NH₂, C₁₋₃ alkyl, C₁₋₃ haloalkyl, C₁₋₃ alkoxy, C₁₋₃ haloalkoxy and C₁₋₃ hydroxyalkoxy groups;
R² is H, D, C₁₋₆ alkyl, C₂₋₆ alkenyl, C₂₋₆ alkynyl, C₃₋₈ cycloalkyl, heterocyclic group consisting of 3-8 atoms, C₆₋₁₀ aryl or heteroaryl consisting of 5-12 atoms, wherein said C₁₋₆ alkyl, C₂₋₆ alkenyl, C₂₋₆ alkynyl, C₃₋₈ cycloalkyl, 3-heterocyclic group composed of 8 atoms, C₆₋₁₀ aryl group and heteroaryl group composed of 5-12 atoms are independently and optionally substituted by 1, 2, 3, 4, or 5 substituents selected from D, F, Cl, Br, I, —NO₂, —CN, —OH, —NH₂, C₁₋₃ alkyl, C₁₋₃ haloalkyl, C₁₋₃ alkoxy, C₁₋₃ haloalkoxy and C₁₋₃ hydroxyalkoxy groups;
R³ is H, D, F, Cl, Br, I. —NO₂, —CN, —OH, —NH₂, C₁₋₆ alkyl, C₁₋₆ haloalkyl, C₁₋₆ alkoxy, or C₁₋₆ alkylamino, wherein said C₁₋₆ alkyl and C₁₋₆ alkoxy are independently and optionally substituted by 1, 2, 3, 4, or 5 substituents selected from D, F, Cl, Br, I, —NO₂, —CN, —OH, —NH₂, oxo, C₁₋₆ alkyl, C₁₋₆ haloalkyl, C₁₋₆ alkoxy, C₁₋₆ alkylamino or C₃₋₈ cycloalkyl groups;
R⁴ is —OW or —NHR^{c}, wherein each R^{c} is independently C₃₋₈ cycloalkyl, heterocyclyl consisting of 3-8 atoms, heterocyclyl consisting of 9-12 atoms, C₆—0.1 aryl, heteroaryl consisting of 5-12 atoms, —C₁₋₆ alkylene (C₃₋₈ cycloalkyl), —C₁₋₆ alkylene (heteroaryl consisting of 3-8 atoms Cyclic group), —C₁₋₆ alkylene —NR^{d}-(heterocyclic group consisting of 3-8 atoms), —C₁₋₆ alkylene (C₆₋₁₀ aryl) or —C₁₋₆ alkylene groups (heteroaryl groups consisting of 5-12 atoms), wherein the C₃₋₈ cycloalkyl groups, heterocyclic groups consisting of 3-8 atoms, and heterocyclic groups consisting of 9-12 atoms, C₆₋₁₀ aryl, heteroaryl consisting of 5-12 atoms, —C₁₋₆ alkylene (C₃₋₈ cycloalkyl), —C₁₋₆ alkylene (3-8 atoms), —C₁₋₆ alkylene —NR^{d}-(heterocyclic group consisting of 3-8 atoms), —C₁₋₆ alkylene (C₆₋₁₀ aryl) and —C₁₋₆ alkylene (heteroaryl consisting of 5-12 atoms) is independently optionally substituted by 1, 2, 3, 4, or 5 R^{4a} groups;
R^{d} is H, D, C₁-6 alkyl, C₃₋₈ cycloalkyl or a heterocyclic group consisting of 3-8 atoms;
each of V¹, V², V³, and V⁴ is independently —(CR⁵R⁶)ₙ—, —(CR⁵R⁶)ₙ—O—, —(CR⁵R⁶)ₙ—S—, —(CR⁵R⁶)ₙ—NR—, —(CR⁵R⁶)ₙ—C(═O)—, —(CR⁵R⁶)ₙ—OC(═O)—, —(CR⁵R⁶)ₙ—C(═O)—O—, —(CR⁵R⁶)ₙ—S(═O)— or —(CR⁵R⁶)ₙ—S(═O)₂—;
each R⁵ and R⁶ is independently H, D, F, Cl, Br, I, —NO₂, —CN, —OH, —NH₂, C₁₋₆ alkyl, C₁₋₆ haloalkyl, C₁₋₆ alkoxy or C₃₋₈ cycloalkyl, wherein said C₁₋₆ alkyl and C₃₋₈ cycloalkyl are independently and optionally substituted by 1, 2, 3, 4, or 5 substituents selected from D, F, Cl, Br, I, —NO₂, —CN, —OH, —NH₂, C₁₋₃ alkyl, C₁₋₃ haloalkyl, C₁₋₃ alkoxy, C₁₋₃ Substituted by haloalkoxy and C₁₋₃ hydroxyalkoxy groups; or
R⁵, R⁶ and the carbon atoms they are connected together form a C₃₋₈ cycloalkyl group or a heterocyclic group consisting of 3-8 atoms, wherein the C₃₋₈ cycloalkyl group and the 3-8 heterocyclic group consisting of 3-8 atoms are independently and optionally substituted by 1, 2, 3, 4, or 5 substituents selected from D, F, Cl, Br, I, —NO₂, —CN, —OH, —NH₂, oxo, C₁₋₃ alkyl, C₁₋₃ haloalkyl, C₁₋₃ alkoxy, C₁₋₃ haloalkoxy and C₁₋₃ hydroxyalkoxy;
R¹ is H, D, C₁₋₆ alkyl, C₁₋₆ haloalkyl, or C₃₋₈ cycloalkyl, wherein said C₁₋₆ alkyl and C₃₋₈ cycloalkyl are independently and optionally substituted by 1, 2, 3, 4, or 5 substituents selected from D, F, Cl, Br, I, —NO₂, —CN, —OH, —NH₂, oxo, C₁₋₃ alkyl, C₁₋₃ haloalkyl, C₁₋₃ alkoxy, C₁₋₃ haloalkoxy, C₁₋₃ hydroxyalkoxy and C₃₋₆ cycloalkyl;
R^{x} is H, D, F, Cl, Br, I, —NO₂, —CN, —OH, —NH₂, C₁₋₆ alkyl, C₁₋₆ haloalkyl and C₁₋₆ alkoxy, wherein said C₁₋₆ alkyl and C₁₋₆ alkoxy are independently and optionally substituted by 1, 2, 3, 4, or 5 substituents selected from D, F, Cl, Br, I, —NO₂, —CN, —OH, —NH₂, C₁₋₃ alkyl, C₁₋₃ haloalkyl, C₁₋₃ alkoxy, C₁₋₃ haloalkoxy and C₁₋₃ hydroxyalkoxy groups;
R^{4a} is D, F, Cl, Br, I, —NO₂, —CN, oxo, C₁₋₆ alkyl, C₂₋₆ alkenyl, C₂₋₆ alkynyl, C₁₋₆ haloalkyl, C₁₋₆ alkoxy, C₁₋₆ alkylamino, C₃₋₈ cycloalkyl, heterocyclic group consisting of 3-8 atoms, —OR⁸, —C(═O)R⁸, —C(═O)OR⁸, —NR⁸R⁹, —C(═O)NR⁸R⁹, —C(═O)NRS(═O)₂R⁹, —NR¹S(═O)₂R⁹, —S(═O)₂R⁸ or —S(═O)₂NR⁸R⁹, wherein the C₁₋₆ alkyl, C₂₋₆ alkenyl, C₂₋₆ alkynyl, C₁₋₆ alkoxyl group, C₁₋₆ alkylamino group, C₃₋₈ cycloalkyl group and heterocyclic group composed of 3-8 atoms are independently and optionally substituted by 1, 2, 3, 4, or 5 substituents selected from D, F, Cl, Br, I, —NO₂, —CN, —OH, —NH₂, C₁₋₃ alkyl, C₁₋₃ haloalkyl, C₁₋₃ alkoxy, C₁₋₃ alkylamino, C₁₋₃ haloalkoxy and C₁₋₃ hydroxyalkoxy groups;
each R⁸ and R⁹ are independently H, D, C₁₋₆ alkyl, C₃₋₈ cycloalkyl, heterocyclic group consisting of 3-8 atoms, C₆₋₁₀ aryl or heteroaryl group consisting of 5-12 atoms, wherein the C₁₋₆ alkyl group, C₃₋₈ cycloalkyl group, heterocyclic group consisting of 3-8 atoms, C₆₋₁₀ aryl group or heteroaryl group consisting of 5-12 atoms are independently and optionally substituted by 1, 2, 3, 4, or 5 substituents selected from D, F, Cl, Br, I, oxo, —NO₂, —CN, —OH, —NH₂, —COOMe, and —COOH;
each n is independently 0, 1 or 2;
or are as defined in WO 2023/116748, the entirety of which is herein incorporated by reference.

In some embodiments, the present invention provides a compound of formula II-ee:

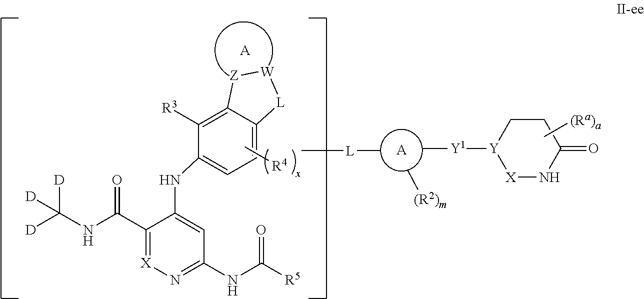

or a pharmaceutically acceptable salt thereof, wherein each of the variables in

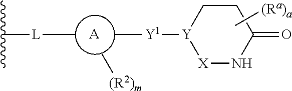

is as defined in the formulae above (e.g., formula I-a) and described in embodiments herein, both singly and in combination, and wherein:
z is selected from carbon atom or nitrogen atom;
w is selected from carbon atom or nitrogen atom;
L is selected from —O(CH)₂)ₘ—, —(CH₂)ₙ—, —NH—(CH₂)ₘ—, —(CH₂)ₙO(CH₂)ₘ—, —(CH₂)ₙ—NH—(CH₂)ₘ—;
Y is selected from CR² or N;
Ring A is heteroaryl or heterocyclyl, preferably 5-10 membered heteroaryl or 3-10 membered heterocyclyl, further preferably 5-6 membered heteroaryl; which is optionally further selected from R¹ is substituted with one or more groups;
R¹ selected from hydrogen, deuterium, halogen, amino, nitro, hydroxy, mercapto, cyano, oxo, thio, alkyl, alkoxy, alkenyl, alkynyl, cycloalkyl, heterocyclyl, aryl, heteroaryl, —(CH)₂)_{q}C(O)R^{c}, —(CH₂)C(O)NR^{a}R^{b}, —(CH₂)_{q}NR^{a}R^{b}, —(CH₂)_{q}NR^{b}C(O)R^{c}, wherein the alkyl, alkoxy, alkenyl, alkynyl, cycloalkyl, heterocyclyl, aryl, heteroaryl is optionally further substituted with one or more groups selected from deuterium, halogen, amino, nitro, hydroxy, mercapto, cyano, oxo, thio, alkyl, alkoxy, alkenyl, alkynyl, cycloalkyl, heterocyclyl, aryl, heteroaryl;
R² is selected from hydrogen, deuterium, alkyl, deuterated alkyl, haloalkyl, alkoxy, haloalkoxy, deuterated alkoxy, halogen;
R³ is selected from hydrogen, deuterium, alkyl, deuterated alkyl, haloalkyl, alkoxy, haloalkoxy, deuterated alkoxy, halogen, amino, mercapto, nitro, hydroxy, cyano, alkenyl, alkynyl, cycloalkyl, heterocyclyl, aryl, heteroaryl;
each R⁴ are independently selected from hydrogen, deuterium, alkyl, deuterated alkyl, haloalkyl, alkoxy, haloalkoxy, halogen, amino, hydroxy, mercapto, nitro, cyano, alkenyl, alkynyl, cycloalkyl, heterocyclyl, aryl, or heteroaryl;
R⁵ selected from alkyl, cycloalkyl, heterocyclyl, —NR⁶R⁷, —OR⁶, aryl, or heteroaryl group; the alkyl, cycloalkyl, heterocyclyl, aryl, or heteroaryl is optionally further substituted with one or more groups selected from deuterium, halogen, amino, nitro, cyano, hydroxy, mercapto, oxo, substituted or unsubstituted alkyl, substituted or unsubstituted alkoxy, substituted or unsubstituted hydroxyalkyl, substituted or unsubstituted alkenyl, substituted or unsubstituted alkynyl, substituted or unsubstituted cycloalkyl, substituted or unsubstituted heterocyclyl, substituted or unsubstituted aryl and substituted or unsubstituted heteroaryl;
R⁶ and R⁷ are independently selected from the group consisting of hydrogen, deuterium, halogen, amino, nitro, cyano, hydroxy, alkyl, alkenyl, alkynyl, cycloalkyl, heterocyclyl, aryl, and heteroaryl, wherein the alkyl, alkenyl, alkynyl, cycloalkyl, heterocyclyl, aryl, and heteroaryl are optionally further substituted with one or more groups selected from deuterium, halogen, amino, nitro, cyano, hydroxy, mercapto, oxo, substituted or unsubstituted alkyl, substituted or unsubstituted alkoxy, substituted or unsubstituted hydroxyalkyl, substituted or unsubstituted alkenyl, substituted or unsubstituted alkynyl, substituted or unsubstituted cycloalkyl, substituted or unsubstituted heterocyclyl, substituted or unsubstituted aryl, and substituted or unsubstituted heteroaryl; alternatively, R⁶ and R⁷ together with the atoms to which they are attached, form a heterocyclic group, which is optionally further substituted with one or more groups selected from deuterium, halogen, amino, nitro, cyano, hydroxy, mercapto, oxo, substituted or unsubstituted alkyl, substituted or unsubstituted alkoxy, substituted or unsubstituted hydroxyalkyl, substituted or unsubstituted alkenyl, substituted or unsubstituted alkynyl, substituted or unsubstituted cycloalkyl, substituted or unsubstituted heterocyclic, substituted or unsubstituted aryl and substituted or unsubstituted heteroaryl;
R^{a} and R^{b} are independently selected from the group consisting of hydrogen, deuterium, halogen, amino, nitro, cyano, hydroxy, alkyl, alkenyl, alkynyl, cycloalkyl, heterocyclyl, aryl, and heteroaryl, wherein the alkyl, alkenyl, alkynyl, cycloalkyl, heterocyclyl, aryl, and heteroaryl groups Heteroaryl is optionally further substituted with a member selected from deuterium, halogen, amino, nitro, cyano, hydroxy, mercapto, oxo, substituted or unsubstituted alkyl, substituted or unsubstituted alkoxy, substituted or unsubstituted hydroxyalkyl, —S(O)ₚR^{d}—, —C(O)ₚR^{d}—, —NR^{e}R^{f}. —C(O)NR^{e}R^{f} Substituted or unsubstituted alkenyl, substituted or unsubstituted alkynyl, substituted or unsubstituted cycloalkyl, substituted or unsubstituted heterocyclyl, substituted or unsubstituted aryl, and substituted or unsubstituted heteroaryl;
alternatively, R^{a} and R^{b} are together with the atoms to which they are attached, form a heterocyclic group, which is optionally further substituted with a moiety selected from deuterium, halogen, amino, nitro, cyano, hydroxy, mercapto, oxo, substituted or unsubstituted alkyl, substituted or unsubstituted alkoxy, substituted or unsubstituted hydroxyalkyl, —S(O)ₚR^{d}, —C(O)R^{d}, —NR^{e}R^{f}, —C(O)NR^{e}R^{f}, substituted or unsubstituted alkenyl, substituted or unsubstituted alkynyl, substituted or unsubstituted cycloalkyl, substituted or unsubstituted heterocyclyl, substituted or unsubstituted aryl, and substituted or unsubstituted heteroaryl;
R^{c} is selected from the group consisting of hydrogen, deuterium, halogen, amino, nitro, cyano, hydroxy, alkyl, alkenyl, alkynyl, cycloalkyl, heterocyclyl, aryl, and heteroaryl, wherein the alkyl, alkenyl, alkynyl, cycloalkyl, heterocyclyl, aryl, and heteroaryl are optionally further substituted with a moiety selected from the group consisting of deuterium, halogen, amino, nitro, cyano, hydroxy, mercapto, oxo, substituted or unsubstituted alkyl, substituted or unsubstituted alkoxy, substituted or unsubstituted hydroxyalkyl, —S(O)ₚR^{d}, —C(O)ₚR^{d}, —NR^{e}R^{f}, —C(O)NR^{e}R^{f}, substituted or unsubstituted alkenyl group, a substituted or unsubstituted alkynyl group, a substituted or unsubstituted cycloalkyl group, a substituted or unsubstituted heterocyclic group, a substituted or unsubstituted aryl group, and a substituted or unsubstituted heteroaryl group Substituted;
R^{d} optionally further substituted with one or more groups selected from the group consisting of deuterium, halogen, amino, nitro, cyano, hydroxy, alkyl, alkenyl, alkynyl, cycloalkyl, heterocyclyl, aryl and heteroaryl, wherein said alkyl, alkenyl, alkynyl, cycloalkyl, heterocyclyl, aryl and heteroaryl are optionally further substituted with one or more groups selected from deuterium, halogen, amino, nitro, cyano, hydroxy, mercapto, oxo, substituted or unsubstituted alkyl, substituted or unsubstituted alkoxy, substituted or unsubstituted hydroxyalkyl, substituted or unsubstituted alkenyl, substituted or unsubstituted alkynyl, substituted or unsubstituted cycloalkyl, substituted or unsubstituted heterocyclyl, substituted or unsubstituted aryl and substituted or unsubstituted heteroaryl;
R^{e} and R^{f} are independently selected from the group consisting of hydrogen, deuterium, halogen, amino, nitro, cyano, hydroxy, alkyl, alkenyl, alkynyl, cycloalkyl, heterocyclyl, aryl, and heteroaryl, wherein the alkyl, alkenyl, alkynyl, cycloalkyl, heterocyclyl, aryl, and heteroaryl are optionally further substituted with one or more groups selected from deuterium, halogen, amino, nitro, cyano, hydroxy, mercapto, oxo, substituted or unsubstituted alkyl, substituted or unsubstituted alkoxy, substituted or unsubstituted hydroxyalkyl, substituted or unsubstituted alkenyl, substituted or unsubstituted alkynyl, substituted or unsubstituted cycloalkyl, substituted or unsubstituted heterocyclyl, substituted or unsubstituted aryl, and substituted or unsubstituted heteroaryl;
alternatively, R^{e} and R^{f} together with the atoms to which they are attached, form a heterocyclic group, which is optionally further substituted with one or more groups selected from deuterium, halogen, amino, nitro, cyano, hydroxy, mercapto, oxo, substituted or unsubstituted alkyl, substituted or unsubstituted alkoxy, substituted or unsubstituted hydroxyalkyl, substituted or unsubstituted alkenyl, substituted or unsubstituted alkynyl, substituted or unsubstituted cycloalkyl, substituted or unsubstituted heterocyclic, substituted or unsubstituted aryl and substituted or unsubstituted heteroaryl;
when substituted, the substituents are selected from deuterium, alkyl, deuteroalkyl, haloalkyl, alkoxy, haloalkoxy, hydroxyalkyl, alkylsulfonyl, alkylamino, halogen, amino, nitro, cyano, hydroxy, alkenyl, alkynyl, cycloalkyl, heterocyclyl, aryl, and heteroaryl;
m is an integer from 0 to 6;
n is an integer from 1 to 6;
p is 0, 1, or 2;
q is an integer from 0 to 6;
x is 0, 1, or 2
or are as defined in CN 116284040, the entirety of which is herein incorporated by reference.

In some embodiments, the present invention provides a compound of formula II-ff:

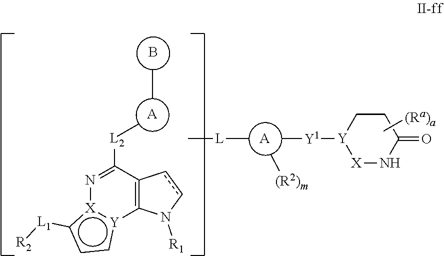

or a pharmaceutically acceptable salt thereof, wherein each of the variables in

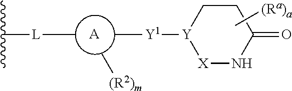

is as defined in the formulae above (e.g., formula I-a) and described in embodiments herein, both singly and in combination, and wherein:
the dotted line represents a single or double bond;
X and Y are independently selected from N or C; and X and Y are not the same;
R₁ is selected from hydrogen, C₁ to C₈ alkoxy, halogen or substituted or unsubstituted C₁ to C₈ alkyl;
L₁ is selected from —O—, —S—, —NR₃—, —CR₃R⁴—, —S(═O)—, —S(═O)₂—, —C(═O)—, —OC(═O)—, —C(═O)O—, —C(═O)NR₃—, —OC(═O)NR₃—, —NR₃C(═O)NR₄—, —NR₃C(═O)— or —NR₃C(═O)O—; L₂ is selected from —O—, —S—, —NR₃—, —CR₃R⁴—, —S(═O)—, —S(═O)₂—, —C(═O)—, —OC(═O)—, —C(═O)O—, —C(═O)NR₃—, —OC(═O)NR₃—, —NR₃C(═O)NR₄—, —NR₃C(═O)— or —NR₃C(═O)O—;
R₃ and R₄ are independently selected from hydrogen, substituted or unsubstituted C₁ to C₈ alkyl group, substituted or unsubstituted C₂ to C₈ alkenyl group, substituted or unsubstituted C₂ to C₈ alkynyl group, Substituted or unsubstituted 3-12-membered cycloalkyl, substituted or unsubstituted 3-12-membered heterocycloalkyl, substituted or unsubstituted 5-12-membered aryl or substituted or unsubstituted 5-12-membered heteroaryl base;
R₂ is selected from a substituted or unsubstituted 3-12-membered cycloalkyl group, a substituted or unsubstituted 3-12-membered heterocycloalkyl group, a substituted or unsubstituted 5-12-membered aryl group or a substituted or unsubstituted 5-12-membered aryl group. 12-membered heteroaryl;
Ring A is selected from substituted or unsubstituted 5-12-membered aryl groups or substituted or unsubstituted 5-12-membered heteroaryl groups;
Ring B is selected from substituted or unsubstituted 5-12-membered aryl groups or substituted or unsubstituted 5-12-membered heteroaryl groups;
The substituents of the alkyl, alkenyl or alkynyl groups are independently selected from deuterium, halogen, hydroxyl, amino, carboxyl, nitro, cyano or C₁ to C₈ alkoxy;
The substituents of the cycloalkyl or heterocycloalkyl are independently selected from deuterium, ester group, halogen, hydroxyl, amino, carboxyl, nitro, cyano, C₁ to C₈ alkoxy or substituted or unsubstituted C₁ to C₈ alkyl; the heteroatoms of the heterocycloalkyl group are selected from N, O or S, and the number of the heteroatoms is 1, 2 or 3;
The substituents of the aryl or heteroaryl groups are independently selected from deuterium, ester group, halogen, hydroxyl, amino, carboxyl, nitro, cyano, C₁ to C₈ alkoxy or substituted or unsubstituted C₁ to C₈ alkyl; alternatively, the aryl or heteroaryl consists of two substituents on the same carbon atom ═O; the heteroatom of the heteroaryl is selected from N, O or S, and the heteroatom number is 1, 2 or 3,
or are as defined in WO 2023/169336, the entirety of which is herein incorporated by reference.

In some embodiments, the present invention provides a compound of formula II-gg:

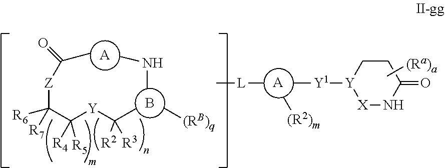

or a pharmaceutically acceptable salt thereof, wherein each of the variables in

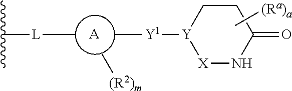

is as defined in the formulae above (e.g., formula I-a) and described in embodiments herein, both singly and in combination, and wherein:
Ring A is a 6,5 or 6,6 fused bicyclic heteroaryl containing two, three, or four ring nitrogen;
R^{A} is independently selected for each occurrence from the group consisting of hydrogen, halogen, —NR^{a}R^{b}, C₁-C₆ alkyl, and C₁-C₆ alkoxy; wherein C₁-C₆ alkyl and C₁-C₆ alkoxy may optionally be substituted by one or more halogen or deuterium;
Ring B is a 6,5 or 6,6 fused bicyclic heteroaryl containing one, two or three ring nitrogen;
R^{B} is independently selected for each occurrence from the group consisting of hydrogen, halogen, —NR^{a}R^{b}, C₁-C₆ alkyl, and C₁-C₆ alkoxy; wherein C₁-C₆ alkyl and C₁-C₆ alkoxy may optionally be substituted by one or more halogens or deuterium;
Y is selected from the group consisting of O, N(R^{a}), S(O)_{w}, CH₂ and a bond;
Z is selected from the group consisting of N(R^{a}) and O;
R² and R⁵ are each independently selected from the group consisting of hydrogen, deuterium, and C₁-C₆ alkyl optionally substituted by one or more substituents each independently selected from R^{P}; or:
   R² and R³, together with the carbon to which they are attached, may be joined together to form a C₃-C₆ cycloalkyl optionally substituted by one or more substituents each independently selected from R^{P};
R⁴ and R⁵ are each independently selected for each occurrence from the group consisting of hydrogen, deuterium, and C₁-C₆ alkyl optionally substituted by one or more substituents each independently selected from R^{P}; or:
   R⁴ and R⁵, together with the carbon to which they are attached, may be joined together to form a C₃-C₆ cycloalkyl optionally substituted by one or more substituents each independently selected from R^{P};
R⁶ and R⁷ are independently selected from the group consisting of hydrogen, deuterium, halogen, hydroxyl, —NR^{a}R^{b}, cyano, oxo, C₁-C₆alkyl, C₂-C₆alkenyl, C₂-C₆alkynyl, C₁-C₆ heteroalkyl, and C₁-C₆alkoxy;
wherein R may be substituted on an available carbon by hydroxyl or one or more substituents each independently selected from R^{P}; or:
   R⁶ and R⁷, together with the carbon to which they are attached, may be joined together to form a C₃-C₆ cycloalkyl optionally substituted by one or more substituents each independently selected from R^{P};
R^{P} is independently selected for each occurrence from the group consisting of deuterium, halogen, hydroxyl, —NR^{a}R^{b}, cyano, oxo, C₁-C₆ alkyl, and C₁-C₆ alkoxy;
R^{a} and R^{b} are independently selected for each occurrence from the group consisting of hydrogen and C₁-C₆ alkyl, wherein C₁-C₆ alkyl may optionally be substituted by one or more halogen or deuterium;
m is 1 or 2;
n is 0 or 1;
p is 0, 1, or 2;
q is 0, 1 or 2; and
w is 0, 1 or 2,
or are as defined in WO 2023/178235, the entirety of which is herein incorporated by reference.

In some embodiments, the present invention provides a compound of formula II-hh:

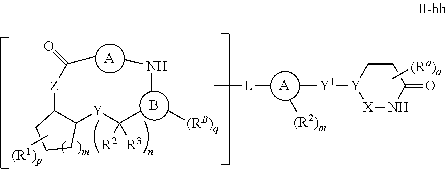

or a pharmaceutically acceptable salt thereof, wherein each of the variables in

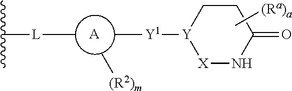

is as defined in the formulae above (e.g., formula I-a) and described in embodiments herein, both singly and in combination, and wherein:
Ring A is a 6,5 or 6,6 fused bicyclic heteroaryl containing two, three, or four ring nitrogens;
R_{A} is independently selected for each occurrence from the group consisting of hydrogen, halogen, —NR^{a}R^{b}, C₁-C₆ alkyl, and C₁-C₆ alkoxy; wherein C₁-C₆ alkyl and C₁-C₆ alkoxy may optionally be substituted by one or more halogen or deuterium;
Ring B is a 6,5 or 6,6 fused bicyclic heteroaryl containing one, two or three ring nitrogen;
R^{B} is independently selected for each occurrence from the group consisting of hydrogen, halogen, —NR^{a}R^{b}, C₁₋₆ alkyl, and C₁₋₆ alkoxy; wherein C₁₋₆ alkyl and C₁₋₆alkoxy may optionally be substituted by one or more halogens or deuterium;
Y is selected from the group consisting of 0, N(Rₐ), S(O)_{w}, CH₂ and a bond;
Z is selected from the group consisting of N(Rₐ) and O;
R¹ is independently selected for each occurrence from the group consisting of deuterium, halogen, hydroxyl, —NR^{a}R^{b}, cyano, C₁-C₆ alkyl, C₂-C₆ alkenyl, C₂-C₆ alkynyl, C₁-C₆ heteroalkyl, and C₁-C₆ alkoxy; wherein R may be substituted on an available carbon by hydroxyl or one or more halogen;
R² and R³ are each independently selected from the group consisting of hydrogen, deuterium, and C₁-C₆ alkyl optionally substituted by one or more halogens; or
R² and R³, together with the carbon to which they are attached, may be joined together to form a C₁-C₆ cycloalkyl;
R^{a} and R^{b} are independently selected for each occurrence from the group consisting of hydrogen and C₁-C₆alkyl, wherein C₁-C₆alkyl may optionally substituted by one or more halogen or deuterium;
m is 1 or 2;
n is 0 or 1;
p is 0, 1, 2, 3 or 4; and
w is 0, 1 or 2,
or are as defined in WO 2023/178234, the entirety of which is herein incorporated by reference.

In some embodiments, the present invention provides a compound of formula II-ii:

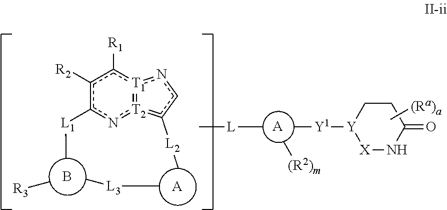

or a pharmaceutically acceptable salt thereof, wherein each of the variables in

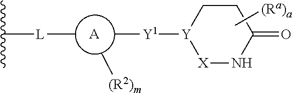

is as defined in the formulae above (e.g., formula I-a) and described in embodiments herein, both singly and in combination, and wherein:
L₁ is selected from single bond and NH;
L₂ is selected from —NH—C(═O)—;
L₃ is selected from —O—, —NH—, —C₁₋₃ alkyl-O—, —C₁₋₃ alkyl-NH— and —C₁₋₃ alkyl-OC₁₋₃ alkyl-, wherein the —C₁₋₃ alkyl-O—, —C₁₋₃ alkyl-NH— and —C₁₋₃ alkyl-OC₁₋₃ alkyl- are optionally substituted by 1, 2 or 3R_{c} groups;
R₁ is selected from —NH—C₁₋₃ alkyl, R² is selected from H;
alternatively, R₁ and R₂ and the atoms to which they are connected together constitute a 5-6 membered heterocyclic alkenyl group;
R₃ is selected from H, F, Cl, Br, I, OH, CN, NH₂, C₁₋₄ alkyl, C₁₋₃ alkoxy and 6-membered heteroaryl, the C₁₋₄ alkyl, C₁₋₃ alkoxy group and 6-membered heteroaryl group are optionally substituted by 1, 2 or 3 Rₐ groups;
Ring A does not exist;
alternatively, Ring A is selected from C₃₋₅ cycloalkyl, 5-6 membered heterocycloalkyl and 5-6 membered heteroaryl, wherein said C₃₋₅ cycloalkyl, 5-6 membered heterocycloalkyl and 5-6 membered heteroaryl group is optionally substituted by 1, 2 or 3 R_{b} groups;
Ring B is selected from phenyl, 5-6 membered heteroaryl, 5-6 membered heterocycloalkyl, pyridopyrrolyl, benzoxazolyl, benzopyrrolyl, benzimidazolyl, and benzopyrazolyl;
Ring C is selected from C₅₋₆ cycloalkyl, C₅₋₆ cycloalkenyl, 5-6 membered heterocycloalkyl and 5-6 membered heterocycloalkenyl;
each Rₐ is independently selected from F, Cl, Br, I, CH₃ and OCH₃;
each R_{b} is independently selected from F, Cl, Br, I, OH, C₁₋₃ alkyl and C₁₋₃ alkoxy, wherein the C₁₋₃ alkyl and C₁₋₃ alkoxy are optionally substituted by 1, 2 or 3 halogen;
alternatively, 2R_{b} and the carbon atoms to which they are connected together constitute a C₃₋₅ cycloalkyl group;
each R_{c} is independently selected from C₁₋₃ alkyl and phenyl, or are as defined in WO 2023/208244, the entirety of which is herein incorporated by reference.

In some embodiments, the present invention provides a compound of formula II-jj:

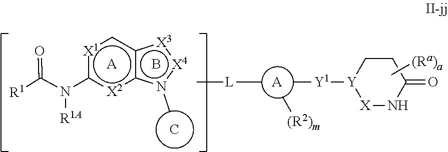

or a pharmaceutically acceptable salt thereof, wherein each of the variables in

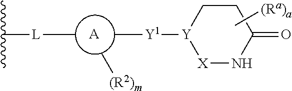

is as defined in the formulae above (e.g., formula I-a) and described in embodiments herein, both singly and in combination, and wherein:
Ring A is an aromatic or heteroaromatic ring fused with ring B that is a 5-membered heteroaromatic ring;
X¹ is N or CH;
X² is N or CR²;
X³ is N or CR³;
X⁴ is N or CR⁴;
Ring C is phenyl, 5 or 6 membered monocyclic heterocyclyl, or 5 to 6 membered heteroaryl, each of which is optionally substituted by one or more R^{C};
each R^{c} is independently halo, —CN, —NR^{N1}R^{N2}, —NR^{N3}C(O)R⁷, —NR^{N4}SO₂R⁷, —C(O)R⁷, —SO₂R⁷, —OR^{O1}, C1-6 alkyl, alkenyl, 3 to 7 membered monocyclic carbocyclyl, phenyl, 5 to 12 membered monocyclic or bicyclic heteroaryl, or 4 to 9 membered monocyclic or bicyclic heterocyclyl, wherein the C1-6 alkyl, 3 to 7 membered monocyclic carbocyclyl, phenyl, 5 to 12 membered monocyclic or bicyclic heteroaryl, and 4 to 9 membered monocyclic or bicyclic heterocyclyl represented by Rc are each optionally substituted with one or more R^{C3}, or two R^{C} taken together with intervening atoms form a 3 to 7 membered monocyclic carbocyclyl optionally substituted with one or more halo;
each R^{Cl} is independently halo, oxo, —CN, —OR^{O1}, —NR^{N1}R^{N2}, —NR^{N3}C(O)R⁷, —C(O)OR^{O3}, —SO₂R⁷, C1-6 alkyl, C3-6 cycloalkyl, phenyl, 5 to 12 membered monocyclic or bicyclic heteroaryl, or 4 to 7 membered monocyclic heterocyclyl, wherein the C1-6 alkyl, phenyl, 5 to 6 membered heteroaryl and 4 to 7 membered monocyclic heterocyclyl represented by R^{c} are each optionally substituted by one or more substituents independently selected from halo, oxo, —CN, —OR^{O1}, —NR^{N1}R^{N2}, C1-6 alkyl, C1-4 haloalkyl, phenyl, 5 to 6 membered heteroaryl, 3 to 7 membered monocyclic carbocyclyl and 4 to 8 membered monocyclic heterocyclyl;
R¹ is H, C1-6 alkyl, —OR^{1A}, —NR^{N1}R^{N2}, C3-6 cycloalkyl, phenyl, 5 to 6 membered heteroaryl, 3 to 7 membered monocyclic carbocyclyl or 4 to 7 membered monocyclic heterocyclyl, wherein the C1-6 alkyl, phenyl, C3-6 cycloalkyl, 5 to 6 membered heteroaryl, 3 to 7 membered monocyclic carbocyclyl and 4 to 7 membered monocyclic heterocyclyl represented by 1e are each optionally substituted by one or more R¹;
R^{1A} is H or C1-3 alkyl;
or R¹ and R^{1A} together with the atom from which they are attached form a 5 or 6 membered monocyclic heterocycle;
R² is H or halo;
R3 is H, —NR^{N1}R^{N2}, —CN, halo, —C(O)R⁷, —C(O)OR^{O3}, —SO₂R⁷, —OR^{O4}, C1-6 alkyl, alkenyl, phenyl, 5 to 6 membered heteroaryl, 3 to 7 membered monocyclic carbocyclyl, or 4 to 9 membered monocyclic or bicyclic heterocyclyl, wherein the C1-6 alkyl, phenyl, 5 to 6 membered heteroaryl, 3 to 7 membered monocyclic carbocyclyl and 4 to 9 membered monocyclic or bicyclic heterocyclyl represented by R³ are each optionally substituted by one or more R⁹;
R⁴ is H or halo;
each R⁷ is independently C1-6 alkyl, phenyl, 5 to 6 membered heteroaryl, 3 to 7 membered monocyclic carbocyclyl or 4 to 7 membered monocyclic heterocyclyl; wherein the C1-6 alkyl, phenyl, 5 to 6 membered heteroaryl, 3 to 7 membered monocyclic carbocyclyl and 4 to 7 membered monocyclic heterocyclyl represented by R⁷ are each optionally substituted by one or more substituents independently selected from halo, oxo, —CN, —OR^{O1}, —NR^{N1}R^{N2}, C1-6 alkyl, C1-4 haloalkyl, 3 to 7 membered monocyclic carbocyclyl and 4 to 7 membered monocyclic heterocyclyl;
each R⁷ is independently halo, oxo, —CN, —OR^{O1}, C1-6 alkyl, C1-4 haloalkyl, phenyl, 5 to 6 membered heteroaryl, 3 to 7 membered monocyclic carbocyclyl or 4 to 7 membered monocyclic heterocyclyl;
each R⁹ is independently halo, oxo, —OR^{O1}, —NR^{N1}R^{N2}, —CN, —C(O)OR³, —SO₂R¹⁰, C1-6 alkyl, phenyl, 5 to 6 membered heteroaryl, 3 to 7 membered monocyclic carbocyclyl or 4 to 10 membered monocyclic or bicyclic heterocyclyl, wherein the C1-6 alkyl, phenyl, 5 to 6 membered heteroaryl, 3 to 7 membered monocyclic carbocyclyl and 4 to 10 membered monocyclic or bicyclic heterocyclyl represented by R⁹ are each optionally substituted by one or more substituents independently selected from halo, oxo, —CN, —OR¹, —NR^{N1}R^{N2}, C1-6 alkyl, C1-4 haloalkyl, phenyl, 5 to 6 membered heteroaryl, 3 to 7 membered monocyclic carbocyclyl and 4 to 7 membered monocyclic heterocyclyl;
each R^{O1} is independently H, C1-6 alkyl, phenyl, 5 to 6 membered heteroaryl, 3 to 7 membered monocyclic or bicyclic carbocyclyl, or 4 to 7 membered monocyclic or bicyclic heterocyclyl, wherein the C1-6 alkyl, phenyl, 5 to 6 membered heteroaryl, 3 to 7 membered monocyclic or bicyclic carbocyclyl, and 4 to 7 membered monocyclic or bicyclic heterocyclyl represented by R^{O1} are each optionally substituted by one or more R^{O2};
each R^{O2} is independently halo, OH, —CN, C1-4 alkoxy, C1-4 alkyl, phenyl, 5 to 6 membered heteroaryl, 3 to 7 membered monocyclic carbocylyl or 4 to 7 membered monocyclic or bicyclic heterocyclyl, wherein the C1-4 alkyl, phenyl, 5 to 6 membered heteroaryl, 3 to 7 membered monocyclic carbocylyl and 4 to 7 membered monocyclic or bicyclic heterocyclyl are each optionally substituted with one or more halo, C1-6 alkyl or —OC1-6 alkyl;
each R^{O3} is independently H, C1-6 alkyl, C1-4 haloalkyl, phenyl, 5 to 6 membered heteroaryl, 3 to 7 membered monocyclic carbocyclyl or 4 to 7 membered monocyclic heterocyclyl, wherein the C1-6 alkyl, phenyl, 5 to 6 membered heteroaryl, 3 to 7 membered monocyclic carbocyclyl and 4 to 7 membered monocyclic heterocyclyl represented by R^{O3} are each optionally substituted by one or more R^{O2};
R^{O4} is H, C1-6 alkyl, phenyl, 5 to 6 membered heteroaryl, 3 to 7 membered monocyclic carbocyclyl or 4 to 7 membered monocyclic heterocyclyl, wherein the C1-6 alkyl, phenyl, 5 to 6 membered heteroaryl, 3 to 7 membered monocyclic carbocyclyl and 4 to 7 membered monocyclic heterocyclyl represented by R^{O4} are each optionally substituted by one or more substituents independently selected from halo, oxo, —CN, —OR^{O1}, —NR^{N1}R^{N2}, alkyl, C1-4 haloalkyl, phenyl, 5 to 6 membered heteroaryl, 3 to 7 membered monocyclic carbocyclyl and 4 to 7 membered monocyclic heterocyclyl;
R^{N1} and R^{N2} are each independently H, C1-6 alkyl, 4 to 7 membered monocyclic heterocyclyl, 5 or 6 membered heteroaryl, or C3-6 cycloalkyl, wherein the C1-6 alkyl represented by R^{N1} and R^{N2} are each optionally substituted with C1-4 alkoxy or phenyl, and wherein the C3-6 cycloalkyl, 4 to 7 membered monocyclic heterocyclyl, 5 or 6 membered heteroaryl represented by R^{N1} and R^{N2} are each optionally substituted with C1-4 alkyl;
each R^{N3} is independently H or C1-6 alkyl; and
each R^{N4} is independently H or C1-6 alkyl or are as defined in WO 2023/220046, the entirety of which is herein incorporated by reference.

In some embodiments, the present invention provides a compound of formula II-kk:

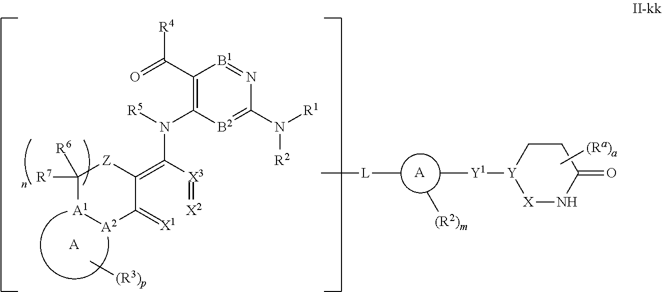

or a pharmaceutically acceptable salt thereof, wherein each of the variables in

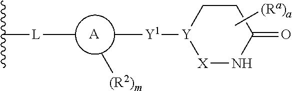

is as defined in the formulae above (e.g., formula I-a) and described in embodiments herein, both singly and in combination, and wherein:
Ring A is an unsubstituted or substituted carbocyclic ring wherein A¹ and A² are both C, or an unsubstituted or substituted 5- or 6-membered heterocyclic ring wherein A¹ and A² are independently N or C, wherein if Ring A is substituted then Ring A is substituted with p instances of R⁸;
each R⁸ is independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ deuteroalkyl, unsubstituted or substituted C₁-C₆ fluoroalkyl, unsubstituted or substituted C₁-C₆ heteroalkyl, unsubstituted or substituted carbocycle, unsubstituted or substituted heterocycle, —CN, —OH, —OR¹⁷, —C(═O)R¹⁶, —CO₂R¹⁶, —C(═O)N(R¹⁶)₂, —N(R¹⁶)₂, —NR¹⁶C(═O)R¹⁷, —SR¹⁶, —S(═O)R¹⁷, —SO₂R¹⁷, or —SO₂N(R¹⁶)₂; wherein if R¹ is attached to a nitrogen atom, then R⁸ is hydrogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ deuteroalkyl, unsubstituted or substituted C₁-C₆ fluoroalkyl, unsubstituted or substituted C₁-C₆ heteroalkyl, unsubstituted or substituted carbocycle, unsubstituted or substituted heterocycle, —C(═O)R¹⁶, —CO₂R¹⁶, —C(═O)N(R¹⁶)₂, —S(═O)R¹⁷, —SO₂R¹⁷, or —SO₂N(R¹⁶)₂; or two R⁸ attached to the same carbon atom are taken together to form ═O, ═S, or ═NH;
Z is —NR¹⁰—, —O—, —S—, —S(═O)—, or —SO₂—;
R¹⁰ is hydrogen, C₁-C₆ alkyl, C₁-C₆ deuteroalkyl, C₁-C₆ fluoroalkyl, C₃-C₆ cycloalkyl, or monocyclic heterocycle;
X¹, X², and X³ are each independently CR^{1′} or N;
each R¹¹ is independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted carbocycle, unsubstituted or substituted heterocycle, —CN, —OH, —OR¹⁷, —C(═O)R¹⁶, —CO₂R¹⁶, —C(═O)N(R¹⁶)₂, —N(R¹⁶)₂, —NR¹⁶C(═O)R¹⁷, —SR¹⁶, —S(═O)R¹⁷, —SO₂R⁷, or —SO₂N(R¹⁶)₂;
B¹ is Nor C R^{12a};
B² is N or CR^{12b};
R^{12a} and R^{12b} are each independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted carbocycle, unsubstituted or substituted heterocycle, —CN, —OH, —OR¹⁷, —C(═O)R¹⁶, —CO₂R¹⁶, —C(═O)N(R⁶)₂, —N(R¹⁶)₂, —NR¹⁶C(═O)R¹⁷, —SR¹⁶, —S(═O)R¹⁷, —SO₂R¹⁷, or —SO₂N(R⁶)₂;
R¹ is hydrogen, C₁-C₆ alkyl, or C₁-C₆ fluoroalkyl;
R² is a Ring B that is an unsubstituted or substituted heterocycle or unsubstituted or substituted carbocycle, wherein if Ring B is substituted then Ring B is substituted with q instances of R¹³;
each R¹³ is independently halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted carbocycle, unsubstituted or substituted heterocycle, —CN, —OH, —OR¹⁷, —C(═O)R¹⁶, —CO₂R¹⁶, —C(═O)N(R⁶)₂, —N(R¹⁶)₂, —NR¹⁶C(═O)R¹⁷, —SR¹⁶, —S(═O)R¹⁷, —SO₂R¹⁷, or —SO₂N(R¹⁶)₂; or two R¹³ groups on adjacent atoms of Ring B are taken together with the intervening atoms to which they are attached to form an unsubstituted or substituted 5- or 6-membered monocyclic carbocycle or an unsubstituted or substituted 5- or 6-membered monocyclic heterocycle; or R² is —C(═O)R¹⁴, —C(═O)NR¹⁴R¹⁵, or —C(═O)OR¹⁴;
R¹⁴ is hydrogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ heteroalkyl, unsubstituted or substituted monocyclic carbocycle, unsubstituted or substituted bicyclic carbocycle, unsubstituted or substituted monocyclic heterocycle, or unsubstituted or substituted bicyclic heterocycle;
R¹⁵ is hydrogen, C₁-C₆ alkyl, or C₁-C₆ fluoroalkyl; or R¹⁴ and R¹⁵ are taken together with the intervening atoms to which they are attached to form an unsubstituted or substituted 4- to 6-membered monocyclic heterocycle; or R¹ and R¹⁵ are taken together with the intervening atoms to which they are attached to form an unsubstituted or substituted 5- or 6-membered monocyclic heterocycle;
R⁴ is hydrogen, C₁-C₆ alkyl, C₁-C₆ heteroalkyl, C₁-C₆ deuteroalkyl, C₁-C₆ fluoroalkyl, or C₃-C₆ cycloalkyl; or R⁴ and R^{12a} are taken together with the intervening atoms to which they are attached to form a substituted or unsubstituted C₅-C₆ cycloalkyl;
R⁵ is hydrogen, C₁-C₆ alkyl, C₁-C₆ fluoroalkyl, C₃-C₆ cycloalkyl, or monocyclic heterocycle;
each R⁶ and R⁷ is independently hydrogen, deuterium, halogen, C₁-C₆ alkyl, C₁-C₆ deuteroalkyl, C₁-C₆ fluoroalkyl, C₁-C₆ heteroalkyl, C₃-C₆ cycloalkyl, monocyclic heterocycle, —CN, —OH, —OR¹⁷, —C(═O)R¹⁶, —CO₂R¹⁶, —C(═O)N(R¹⁶)₂, —N(R¹⁶)₂, —NR¹⁶C(═O)R¹⁷, —SR¹⁶, —S(═O)R¹⁷, —SO₂R¹⁷, or —SO₂N(R¹⁶)₂; or one R⁶ and one R⁷ attached to the same carbon atom are taken together with the carbon atom to which they are attached to form C═O or a C₃-C₆ cycloalkane;
each R¹⁶ is independently hydrogen, substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₇ cycloalkyl, substituted or unsubstituted monocyclic 3- to 8-membered heterocycloalkyl, substituted or unsubstituted phenyl, or substituted or unsubstituted monocyclic heteroaryl; or two R¹⁶ on the same N atom are taken together with the N atom to which they are attached to form a substituted or unsubstituted N-containing heterocycle; and
each R¹⁷ is independently substituted or unsubstituted C₁-C₆ alkyl, substituted or unsubstituted C₁-C₆ fluoroalkyl, substituted or unsubstituted C₁-C₆ heteroalkyl, substituted or unsubstituted C₃-C₇ cycloalkyl, substituted or unsubstituted monocyclic 3- to 8-membered heterocycloalkyl, substituted or unsubstituted phenyl, or substituted or unsubstituted monocyclic heteroaryl; wherein each substituted alkyl, substituted fluoroalkyl, substituted deuteroalkyl, substituted alkoxy, substituted fluoroalkoxy, substituted heteroalkyl, substituted carbocycle, and substituted heterocycle is substituted with one or more R^{s} groups independently selected from the group consisting of deuterium, halogen, C₁-C₆ alkyl, monocyclic carbocycle, monocyclic heterocycle, —CN, —CH₂CN, —OR¹⁸, —CH₂OR⁸, —CO₂R¹⁸, —CH₂CO₂R¹⁸, —C(═O)N(R¹⁸)₂, —CH₂C(═O)N(R⁸)₂, —N(R¹⁸)₂, —CH₂N(R¹⁸)₂, —NR¹⁸C(═O)R¹⁸, —CH₂NR¹⁸C(═O)R¹⁸, —NR⁸SO₂R¹⁹, —CH₂NR¹⁸SO₂R¹⁸, —SR¹⁸, —CH₂SR¹⁸, —S(═O)R¹⁹, —CH₂S(═O)R¹⁹, —SO₂R¹⁹, —CH₂SO₂R¹⁹, —SO₂N(R¹⁸)₂, or —CH₂SO₂N(R¹⁸)₂;
each R¹⁸ is independently selected from hydrogen, C₁-C₆ alkyl, C₁-C₆ fluoroalkyl, C₁-C₆ heteroalkyl, C₃-C₆ cycloalkyl, C₂-C₆ heterocycloalkyl, phenyl, benzyl, 5-membered heteroaryl and 6-membered heteroaryl; or two R¹⁸ groups are taken together with the N atom to which they are attached to form a N-containing heterocycle;
each R¹⁹ is independently selected from C₁-C₆ alkyl, C₁-C₆ heteroalkyl, C₃-C₆ cycloalkyl, C₂-C₆ heterocycloalkyl, phenyl, benzyl, 5-membered heteroaryl, and 6-membered heteroaryl; n is 1, 2, or 3; p is 1, 2, 3, or 4; and q is 0, 1, 2, 3, or 4, or are as defined in WO 2023/227946, the entirety of which is herein incorporated by reference.

In certain embodiments, the present invention provides a compound of formula III:

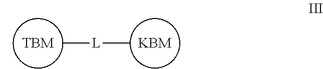

or a pharmaceutically acceptable salt thereof, wherein:
TBM is a TYK2 binding moiety capable of binding to TYK2 protein;
L is a bivalent moiety that connects TBM to KBM; and
KBM is KLHDC2 E3 ubiquitin ligase binding moiety capable of binding to KLHDC2 protein.

In some embodiments, the present invention provides a compound of formula III, wherein TBM-L-is any one of the following:

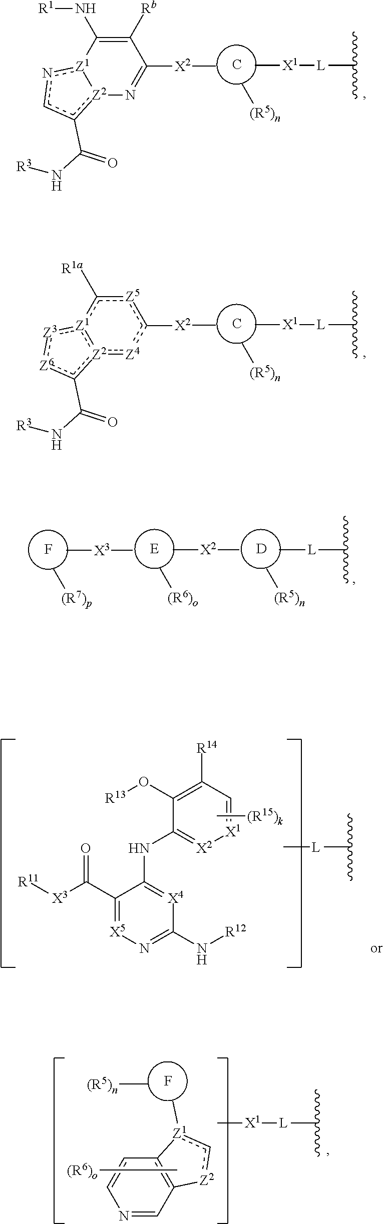

wherein each of the variables is as defined above in formula I-a, I-a′, I-a″, I-b II-cc, and I-c or subgenera thereof and described in embodiments herein, both singly and in combination.

As described above and in certain embodiments, the present invention provides a compound of formula III as a compound of formula III-a:

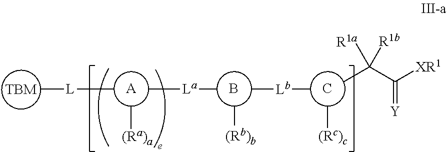

or a pharmaceutically acceptable salt thereof, wherein TBM and L are described and defined herein, and wherein:
R¹, R^{1a} and R^{1b} are each independently hydrogen or optionally substituted C₁₋₆ aliphatic;
each R^{a}, R^{b}, and R^{c} are each independently hydrogen, R^{A}, halogen, —CN, —NO₂, oxo, —OR, thioxo, —SR, —NR₂, —S(O)₂R, —S(O)₂NR₂, —S(O)R, —S(O)(NR)R—P(O)(OR)₂, —P(O)(NR₂)₂, —CFR₂, —CRF₂, —CF₃, —CR₂(OR), —CR₂(NR₂), —C(O)R, —C(O)OR, or —C(O)NR₂;
each R^{A} is independently an optionally substituted group selected from C₁₋₁₀ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom or adjacent atoms are optionally taken together with their intervening atoms to form an optionally substituted 4-11 membered saturated or partially unsaturated carbocyclic or heterocyclic monocyclic, bicyclic, bridged bicyclic, spirocyclic, or heteroaryl ring having 0-3 heteroatoms, in addition to the atom or atoms to which they are attached, independently selected from nitrogen, oxygen, and sulfur;
Ring A is a ring selected from phenyl, naphthyl, a 4-10 membered saturated or partially unsaturated monocyclic or bicyclic carbocyclyl or heterocyclyl having 1-3 heteroatoms independently selected from nitrogen, oxygen, and sulfur, or a 5-10 membered monocyclic or bicyclic heteroaryl having 1-4 heteroatoms independently selected from nitrogen, oxygen, and sulfur;
Ring B is bivalent ring selected from phenyl, a 3-10 membered saturated or partially unsaturated monocyclic, bicyclic, bridged bicyclic, or spirocyclic carbocyclyl or heterocyclyl having 1-3 heteroatoms independently selected from nitrogen, oxygen, and sulfur, or a 5-10 membered monocyclic or bicyclic heteroaryl having 1-4 heteroatoms independently selected from nitrogen, oxygen, and sulfur;
Ring C is bivalent ring selected from phenyl, a 4-13 membered saturated or partially unsaturated monocyclic, bicyclic, bridged bicyclic, or spirocyclic carbocyclyl or heterocyclyl having 1-4 heteroatoms independently selected from nitrogen, oxygen, and sulfur, or a 5-10 membered monocyclic or bicyclic heteroaryl having 1-4 heteroatoms independently selected from nitrogen, oxygen, and sulfur;
each of L and L^{b} is independently a covalent bond or a C₁₋₃ bivalent straight or branched saturated or unsaturated hydrocarbon chain wherein 1-3 methylene units of the chain are independently and optionally replaced with —O—, —C(O)—, —C(S)—, —C(R)₂—, —CF(R)—, —C(F)₂—, —N(R)—, —S—, —S(O)— or —CR═CR—;
a, b, and c are each independently 0, 1, 2, 3 or 4;
e is 0 or 1;
X is —O—, —N(R)—, or —S—; and
Y is O, N(R), or S.

As defined above and described herein, R¹, R^{1a} and R^{1b} are each independently hydrogen or optionally substituted C₆ aliphatic.

In some embodiments, R¹ is hydrogen. In some embodiments, R¹ is an optionally substituted C₁₋₆ aliphatic. In some embodiments, R^{1a} is hydrogen. In some embodiments, R^{1a} is an optionally substituted C₁₋₆ aliphatic. In some embodiments, R^{1b} is hydrogen. In some embodiments, R^{1b} is an optionally substituted C₁₋₆ aliphatic.

In some embodiments, R¹, R^{1a} and R^{1b} are selected from those depicted in Table 1, below.

As defined above and described herein, each R^{a}, R^{b}, and R^{c} are each independently hydrogen, R^{A}, halogen, —CN, —NO₂, —OR, oxo, —SR, —NR₂, —S(O)₂R, —S(O)₂NR₂, —S(O)R, —S(O)(NR)R—P(O)(OR)₂, —P(O)(NR₂)₂, —CFR₂, —CRF₂, —CF₃, —CR₂(OR), —CR₂(NR₂), —C(O)R, —C(O)OR, or —C(O)NR₂.

In some embodiments, R^{a} is hydrogen. In some embodiments, R^{a} is R^{A}. In some embodiments, Rₐ is halogen. In some embodiments, R^{a} is —CN. In some embodiments, R^{a} is —NO₂. In some embodiments, R^{a} is —OR. In some embodiments, R^{a} is oxo. In some embodiments, R^{a} is —SR. In some embodiments, Rₐ is —NR₂. In some embodiments, R^{a} is —S(O)₂R. In some embodiments, R^{a} is —S(O)₂NR₂. In some embodiments, R^{a} is —S(O)R. In some embodiments, R^{a} is —S(O)(NR)R. In some embodiments, Rₐ is —P(O)(OR)₂. In some embodiments, R^{a} is —P(O)(NR₂)₂. In some embodiments, R^{a} is —CFR₂. In some embodiments, R^{a} is —CRF₂. In some embodiments, R^{a} is —CF₃. In some embodiments, R^{a} is —CR₂(OR). In some embodiments, R^{a} is —CR₂(NR₂). In some embodiments, R^{a} is —C(O)R. In some embodiments, Rₐ is —C(O)OR. In some embodiments, R^{a} is —C(O)NR₂.

In some embodiments, R^{a} is C₁₋₃ aliphatic, halogen, —CN, —NO₂, —OR, oxo, —SR—NR₂, —S(O)₂R, —S(O)₂NR₂, —S(O)R, —S(O)(NR)R—P(O)(OR)₂, —P(O)(NR₂)₂, —CFR₂, —CRF₂, —CF₃, —CR₂(OR), —CR₂(NR₂), —C(O)R, —C(O)OR, or —C(O)NR₂.

In some embodiments, R^{a} is C₁₋₃ aliphatic, halogen, —CN, —NO₂, —OR, oxo, —SR, —NR₂, —CFR₂, —CRF₂, —CF₃, —CR₂(OR), —CR₂(NR₂), —C(O)R, —C(O)OR, or —C(O)NR₂.

In some embodiments, R^{a} is halogen, C₁₋₆ alkyl, C₃₋₆ cycloalkyl, C₁₋₆ fluoroalkyl, C₃₋₆ fluorocycloalkyl, —OH, oxo, -0C₁₋₆ alkyl, —OC₃₋₆ cycloalkyl, -0C₁₋₆ fluoroalkyl, -0C₃₋₆ fluorocycloalkyl.

In some embodiments, R^{a} is fluoro, chloro, methyl, cyclopropyl, —CF₃, or —OMe.

In some embodiments, R^{b} is hydrogen. In some embodiments, R^{b} is R^{A}. In some embodiments, R^{b} is halogen. In some embodiments, R^{b} is —CN. In some embodiments, R^{b} is —NO₂. In some embodiments, R^{b} is —OR. In some embodiments, R^{b} is oxo. In some embodiments, R^{b} is —SR. In some embodiments, Rₐ is —NR₂. In some embodiments, R^{b} is —S(O)₂R. In some embodiments, R^{b} is —S(O)₂NR₂. In some embodiments, R^{b} is —S(O)R. In some embodiments, R^{b} is —S(O)(NR)R. In some embodiments, R^{b} is —P(O)(OR)₂. In some embodiments, R^{b} is —P(O)(NR₂)₂. In some embodiments, R^{b} is —CFR₂. In some embodiments, R^{b} is —CRF₂. In some embodiments, R^{b} is —CF₃. In some embodiments, R^{b} is —CR₂(OR). In some embodiments, R^{b} is —CR₂(NR₂). In some embodiments, R^{b} is —C(O)R. In some embodiments, R is —C(O)OR. In some embodiments, R^{b} is —C(O)NR₂.

In some embodiments, R^{b} is C₁₋₃ aliphatic, halogen, —CN, —NO₂, —OR, oxo, —SR. —NR₂, —S(O)₂R, —S(O)₂NR₂, —S(O)R, —S(O)(NR)R, —P(O)(OR)₂, —P(O)(NR₂)₂, —CFR₂, —CRF₂, —CF₃, —CR₂(OR), —CR₂(NR₂), —C(O)R, —C(O)OR, or —C(O)NR₂.

In some embodiments, R^{b} is C₁₋₃ aliphatic, halogen, —CN, —NO₂, —OR, oxo. —SR, —NR₂, —CFR₂, —CRF₂, —CF₃, —CR₂(OR), —CR₂(NR₂), —C(O)R, —C(O)OR, or —C(O)NR₂.

In some embodiments, R^{b} is halogen, C₁₋₆ alkyl, C₃₋₆ cycloalkyl, C₁₋₆ fluoroalkyl, C₃₋₆ fluorocycloalkyl, —OC₁₋₆ alkyl, —OC₃₋₆ cycloalkyl, —OC₁₋₆ fluoroalkyl, —OC₃₋₆ fluorocycloalkyl.

In some embodiments, R^{b} is fluoro, chloro, methyl, cyclopropyl, —CF₃, or —OMe.

In some embodiments, R^{c} is hydrogen. In some embodiments, R^{c} is R^{A}. In some embodiments, R^{c} is halogen. In some embodiments, R^{c} is —CN. In some embodiments, R^{c} is —NO₂. In some embodiments, R^{c} is —OR. In some embodiments, R^{c} is oxo. In some embodiments, R^{c} is —SR. In some embodiments, R^{c} is —NR₂. In some embodiments, R^{c} is —S(O)₂R. In some embodiments, R^{c} is —S(O)₂NR₂. In some embodiments, R^{c} is —S(O)R. In some embodiments, R^{c} is —S(O)(NR)R. In some embodiments, R^{c} is —P(O)(OR)₂. In some embodiments, R^{c} is —P(O)(NR₂)₂. In some embodiments, R^{c} is —CFR₂. In some embodiments, R^{c} is —CRF₂. In some embodiments, R^{c} is —CF₃. In some embodiments, R^{c} is —CR₂(OR). In some embodiments, R^{c} is —CR₂(NR₂). In some embodiments, R^{c} is —C(O)R. In some embodiments, R^{c} is —C(O)OR. In some embodiments, R^{c} is —C(O)NR₂.

In some embodiments, R^{c} is C₁₋₃ aliphatic, halogen, —CN, —NO₂, —OR, oxo, —SR. —NR₂, —S(O)₂R, —S(O)₂NR₂, —S(O)R, —S(O)(NR)R—P(O)(OR)₂, —P(O)(NR₂)₂, —CFR₂, —CRF₂, —CF₃, —CR₂(OR), —CR₂(NR₂), —C(O)R, —C(O)OR, or —C(O)NR₂.

In some embodiments, R^{c} is C₁₋₃ aliphatic, halogen, —CN, —NO₂, —OR, oxo, —SR, —NR₂, —CFR₂, —CRF₂, —CF₃, —CR₂(OR), —CR₂(NR₂), —C(O)R, —C(O)OR, or —C(O)NR₂.

In some embodiments, R^{c} is halogen, C₁₋₆ alkyl, C₃₋₆ cycloalkyl, C₁₋₆ fluoroalkyl, C₃₋₆ fluorocycloalkyl, —OC₁₋₆ alkyl, —OC₃₋₆ cycloalkyl, —OC₁₋₆ fluoroalkyl, —OC₃₋₆ fluorocycloalkyl.

In some embodiments, R^{c} is fluoro, chloro, methyl, cyclopropyl, —CF₃, or —OMe.

In some embodiments, R^{a}, R^{b}, and R^{c} are selected from those depicted in Table 1, below.

As defined above and described herein, each R^{A} is independently an optionally substituted group selected from C₁o aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, each R^{A} is independently an optionally substituted group selected from C₁₋₁₀ aliphatic. In some embodiments, each R^{A} is independently an optionally substituted phenyl. In some embodiments, each R^{A} is independently an optionally substituted 3-7 membered saturated or partially unsaturated carbocyclic or heterocyclic ring having 1-2 heteroatoms independently selected from nitrogen, oxygen, and sulfur. In some embodiments, each R^{A} is independently an optionally substituted 5-6 membered heteroaryl ring having 1-4 heteroatoms independently selected from nitrogen, oxygen, and sulfur.

In some embodiments, R^{A} is —(CH₂)₃NH₂. In some embodiments, R^{A} is —(CH₂)₃NHCO₂tBu. In some embodiments, R^{A} is —(CH₂)₆NH₂. In some embodiments, R^{A} is —(CH₂)₆NHCO₂tBu. In some embodiments, R^{A} is —(CH₂)₉NH₂. In some embodiments, R^{A} is —(CH₂)₉NHCO₂tBu. In some embodiments, R^{A} is —(CH₂)₂CO₂H. In some embodiments, R^{A} is —(CH₂)₅CO₂H. In some embodiments, R^{A} is —(CH₂)₆CO₂H. In some embodiments, R^{A} is —(CH₂)₈CO₂H.

In some embodiments, each R^{A} is selected from those depicted in Table 1, below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are optionally taken together with their intervening atom to form an optionally substituted 4-11 membered saturated or partially unsaturated carbocyclic or heterocyclic monocyclic, bicyclic, bridged bicyclic, spirocyclic, or heteroaryl ring having 0-3 heteroatoms, in addition to the atom to which they are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same or adjacent atoms are optionally taken together with their intervening atom or atoms to form an optionally substituted 4-11 membered saturated or partially unsaturated carbocyclic or heterocyclic monocyclic, bicyclic, bridged bicyclic, spirocyclic, or heteroaryl ring having 0-3 heteroatoms, in addition to the atom or atoms to which they are attached, independently selected from nitrogen, oxygen, and sulfur.

In some embodiments, R is selected from those depicted in Table 1, below.

As defined above and described herein, Ring A is a ring selected from phenyl, naphthyl, a 4-10 membered saturated or partially unsaturated monocyclic or bicyclic carbocyclyl or heterocyclyl having 1-3 heteroatoms independently selected from nitrogen, oxygen, and sulfur, or a 5-10 membered monocyclic or bicyclic heteroaryl having 1-4 heteroatoms independently selected from nitrogen, oxygen, and sulfur;

In some embodiments, Ring A is phenyl. In some embodiments, Ring A is naphthyl. In some embodiments, Ring A is a 4-10 membered saturated or partially unsaturated monocyclic or bicyclic carbocyclyl or heterocyclyl having 1-3 heteroatoms independently selected from nitrogen, oxygen, and sulfur. In some embodiments, Ring A is a 5-10 membered monocyclic or bicyclic heteroaryl having 1-4 heteroatoms independently selected from nitrogen, oxygen, and sulfur. In some embodiments, Ring A is 10-membered bicyclic heteroaryl having 1-4 heteroatoms independently selected from nitrogen, oxygen, and sulfur.

In some embodiments, Ring A is selected from phenyl, naphthyl, or a 5-10 membered monocyclic or bicyclic heteroaryl having 1-4 nitrogen atoms. In some embodiments, Ring A is a ring selected from phenyl and naphthyl. In some embodiments, Ring A is

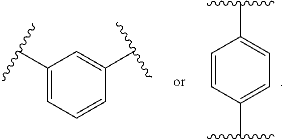 [SMILES: *c1cccc(*)c1 ; *c1ccc(*)cc1]

In some embodiments, Ring A is selected from a 5-10 membered monocyclic or bicyclic heteroaryl having 1-4 nitrogen atoms. In some embodiments, Ring A is a 5-6 membered monocyclic heteroaryl having 1-4 nitrogen atoms. In some embodiments, Ring A is pyridyl. In some embodiments, Ring A is

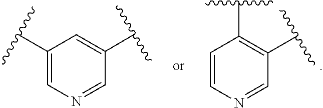 [SMILES: *c1cncc(*)c1 ; *c1ccncc1*]

In some embodiments, Ring A is a 7-10 membered bicyclic heteroaryl having 1-4 nitrogen atoms. In some embodiments, Ring A is indolyl, quinolinyl or isoquinolinyl. In some embodiments, Ring A is indolyl. In some embodiments, Ring A is

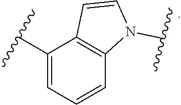 [SMILES: *c1cccc2c1ccn2*]

In some embodiments, Ring A is quinolinyl or isoquinolinyl. In some embodiments, Ring A is

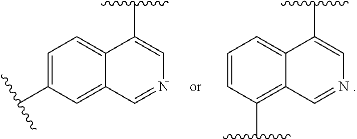 [SMILES: *c1ccc2c(*)cncc2c1 ; *c1cncc2c(*)cccc12]

In some embodiments, Ring A is a 4-10 membered saturated or partially unsaturated monocyclic or bicyclic carbocyclyl or heterocyclyl having 1-3 heteroatoms independently selected from nitrogen, oxygen, and sulfur. In some embodiments, Ring A is a 5-6 membered saturated or partially unsaturated monocyclic carbocyclyl or 5-6 membered heterocyclyl having 1-3 heteroatoms independently selected from nitrogen, oxygen, and sulfur.

In some embodiments, wherein Ring A is cyclohexyl or piperdinyl.

In some embodiments, Ring A is

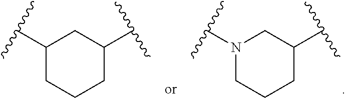 [SMILES: *C1CCCC(*)C1 ; *C1CCCN(*)C1]

In some embodiments, Ring A is a 8-10 membered saturated or partially unsaturated bicyclic carbocyclyl or 8-10 membered saturated or partially unsaturated bicyclic heterocyclyl having 1-3 heteroatoms independently selected from nitrogen, oxygen, and sulfur.

In some embodiments, Ring A is phenyl, pyridyl, or isoquinolinyl.

In some embodiments, Ring A is selected from those depicted in Table 1, below.

As defined above and described herein, Ring B is bivalent ring selected from phenyl, a 3-10 membered saturated or partially unsaturated monocyclic, bicyclic, bridged bicyclic, or spirocyclic carbocyclyl or heterocyclyl having 1-3 heteroatoms independently selected from nitrogen, oxygen, and sulfur, or a 5-10 membered monocyclic or bicyclic heteroaryl having 1-4 heteroatoms independently selected from nitrogen, oxygen, and sulfur.

In some embodiments, Ring B is phenyl. In some embodiments, Ring B is a 3-10 membered saturated or partially unsaturated monocyclic, bicyclic, bridged bicyclic, or spirocyclic carbocyclyl or heterocyclyl having 1-3 heteroatoms independently selected from nitrogen, oxygen, and sulfur. In some embodiments, Ring B is a 5-10 membered monocyclic or bicyclic heteroaryl having 1-4 heteroatoms independently selected from nitrogen, oxygen, and sulfur.

In some embodiments, Ring B is selected from phenyl, naphthyl, or a 5-10 membered monocyclic or bicyclic heteroaryl having 1-4 nitrogen atoms. In some embodiments, Ring B is selected from phenyl and naphthyl. In some embodiments, Ring B is

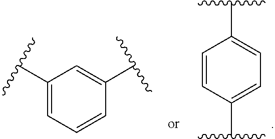 [SMILES: *c1cccc(*)c1 ; *c1ccc(*)cc1]

In some embodiments, Ring B is a ring selected from a 5-10 membered monocyclic or bicyclic heteroaryl having 1-4 nitrogen atoms. In some embodiments, Ring B is a 5-6 membered monocyclic heteroaryl having 1-4 nitrogen atoms. In some embodiments, Ring B is pyridyl. In some embodiments, Ring B is

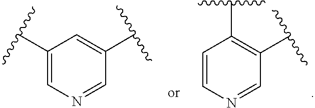 [SMILES: *c1cncc(*)c1 ; *c1ccncc1*]

In some embodiments, Ring B is a 7-10 membered bicyclic heteroaryl having 1-4 nitrogen atoms. In some embodiments, Ring B is indolyl, quinolinyl or isoquinolinyl. In some embodiments, Ring B is indolyl. In some embodiments, Ring B is

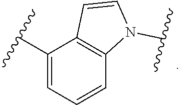 [SMILES: *c1cccc2c1ccn2*]

In some embodiments, Ring B is quinolinyl or isoquinolinyl. In some embodiments, Ring B is

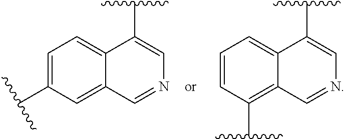 [SMILES: *c1ccc2c(*)cncc2c1 ; *c1cncc2c(*)cccc12]

In some embodiments, Ring B is a 4-10 membered saturated or partially unsaturated monocyclic or bicyclic carbocyclyl or heterocyclyl having 1-3 heteroatoms independently selected from nitrogen, oxygen, and sulfur. In some embodiments, Ring B is a 5-6 membered saturated or partially unsaturated monocyclic carbocyclyl or 5-6 membered heterocyclyl having 1-3 heteroatoms independently selected from nitrogen, oxygen, and sulfur. In some embodiments, Ring B is cyclohexyl or piperdinyl. In some embodiments, Ring B is

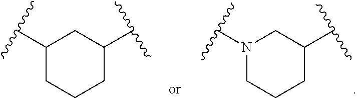 [SMILES: *C1CCCC(*)C1 ; *C1CCCN(*)C1]

In some embodiments, Ring B is a 8-10 membered saturated or partially unsaturated bicyclic carbocyclyl or 8-10 membered saturated or partially unsaturated bicyclic heterocyclyl having 1-3 heteroatoms independently selected from nitrogen, oxygen, and sulfur.

In some embodiments, Ring B is piperdinyl, phenyl, pyridyl, or indolyl.

In some embodiments, Ring B is selected from those depicted in Table 1, below.

As defined above and described herein, Ring C is bivalent ring selected from phenyl, a 4-13 membered saturated or partially unsaturated monocyclic, bicyclic, bridged bicyclic, or spirocyclic carbocyclyl or heterocyclyl having 1-4 heteroatoms independently selected from nitrogen, oxygen, and sulfur, or a 5-10 membered monocyclic or bicyclic heteroaryl having 1-4 heteroatoms independently selected from nitrogen, oxygen, and sulfur.

In some embodiments, Ring C is phenyl. In some embodiments, Ring C is a 4-13 membered saturated or partially unsaturated monocyclic, bicyclic, bridged bicyclic, or spirocyclic carbocyclyl or heterocyclyl having 1-4 heteroatoms independently selected from nitrogen, oxygen, and sulfur. In some embodiments, Ring C is a 5-10 membered monocyclic or bicyclic heteroaryl having 1-4 heteroatoms independently selected from nitrogen, oxygen, and sulfur.

In some embodiments, Ring C is bivalent ring selected from phenyl, a 11-12 membered saturated spirocyclic heterocyclyl having 1-4 heteroatoms independently selected from nitrogen, oxygen, and sulfur, or a 5-6 membered monocyclic heteroaryl having 1-4 heteroatoms independently selected from nitrogen, oxygen, and sulfur.

In some embodiments, Ring C is a ring selected from phenyl, a 4-13 membered saturated or partially unsaturated monocyclic, bicyclic, carbocyclyl or heterocyclyl having 1-4 heteroatoms independently selected from nitrogen, oxygen, and sulfur, or a 5-6 membered monocyclic heteroaryl having 1-4 nitrogen atoms.

In some embodiments, Ring C is a ring selected from phenyl or a 5-6 membered monocyclic heteroaryl having 1-4 nitrogen atoms. In some embodiments, Ring C is

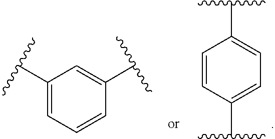 [SMILES: *c1cccc(*)c1 ; *c1ccc(*)cc1]

In some embodiments, Ring C is a 5-6 membered monocyclic heteroaryl having 1-4 nitrogen atoms

In some embodiments, Ring C is pyridyl or pyridonyl. In some embodiments, Ring C is

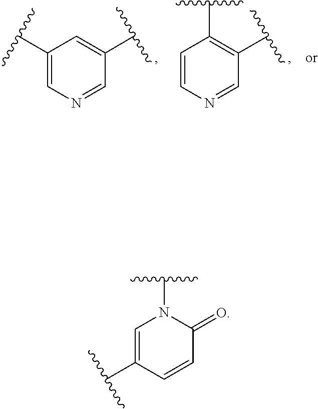 [SMILES: *c1cncc(*)c1 ; *c1ccncc1* ; *c1ccc(=O)n(*)c1]

In some embodiments, Ring C is a 4-10 membered saturated or partially unsaturated monocyclic or bicyclic carbocyclyl or heterocyclyl having 1-3 heteroatoms independently selected from nitrogen, oxygen, and sulfur. In some embodiments, Ring C is a 5-6 membered saturated or partially unsaturated monocyclic carbocyclyl or 5-6 membered heterocyclyl having 1-3 heteroatoms independently selected from nitrogen, oxygen, and sulfur. In some embodiments, Ring C is a 5-6 membered monocyclic heterocyclyl having 1-2 heteroatoms independently selected from nitrogen, oxygen, and sulfur. In some embodiments, Ring C is cyclohexyl or piperdinyl. In some embodiments, Ring C is

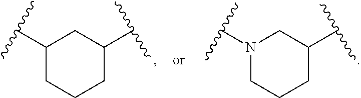 [SMILES: *C1CCCC(*)C1 ; *C1CCCN(*)C1]

In some embodiments, Ring C is phenyl, pyridyl, pyridonyl, or a 12-membered saturated spirocyclic heterocyclyl having 1-4 heteroatoms independently selected from nitrogen, oxygen, and sulfur.

In some embodiments, Ring C is selected from those depicted in Table 1, below.

In some embodiments, Ring A is a ring selected from phenyl, naphthyl, or a 5-10 membered monocyclic or bicyclic heteroaryl having 1-4 nitrogen atoms; Ring B is a ring selected from phenyl, naphthyl, or a 5-10 membered monocyclic or bicyclic heteroaryl having 1-4 nitrogen atoms, or a 4-10 membered saturated or partially unsaturated monocyclic or bicyclic carbocyclyl or heterocyclyl having 1-3 heteroatoms independently selected from nitrogen, oxygen, and sulfur; and Ring C is a 4-10 membered saturated or partially unsaturated monocyclic or bicyclic carbocyclyl or heterocyclyl having 1-3 heteroatoms independently selected from nitrogen, oxygen, and sulfur.

In some embodiments, Ring A is a ring selected from phenyl, naphthyl, or a 5-10 membered monocyclic or bicyclic heteroaryl having 1-2 nitrogen atoms; Ring B is a ring selected from phenyl, naphthyl, or a 5-10 membered monocyclic or bicyclic heteroaryl having 1-2 nitrogen atoms, or a 4-7 membered saturated or partially unsaturated monocyclic carbocyclyl or heterocyclyl having 1-3 heteroatoms independently selected from nitrogen, oxygen, and sulfur; and Ring C is a 6-membered partially unsaturated monocyclic heterocyclyl having 1-3 heteroatoms independently selected from nitrogen, oxygen, and sulfur.

In some embodiments, Ring A and Ring B are each independently selected from:

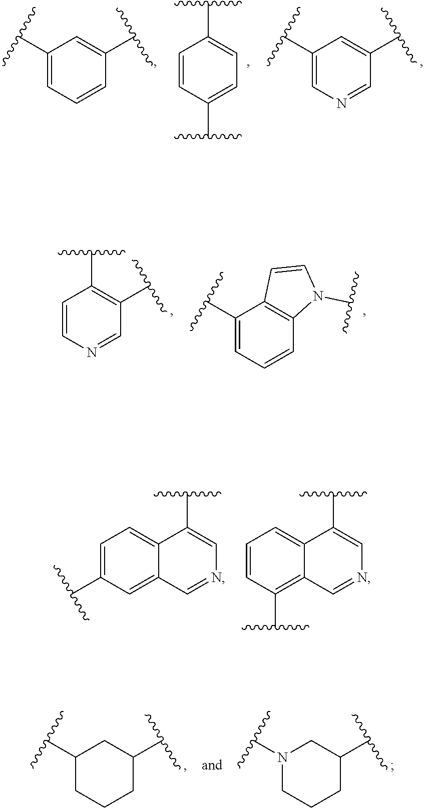

and Ring C is selected from

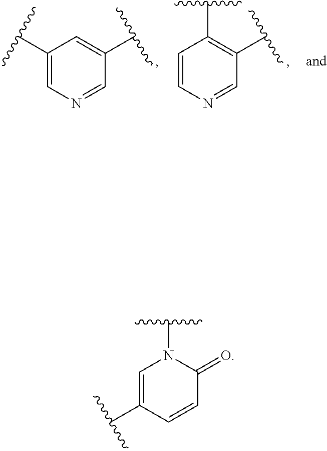 [SMILES: *c1cncc(*)c1 ; *c1ccncc1* ; *c1ccc(=O)n(*)c1]

As defined above and described herein, each of L^{a} and L^{b} are independently a covalent bond or a C₁₋₃ bivalent straight or branched saturated or unsaturated hydrocarbon chain wherein 1-3 methylene units of the chain are independently and optionally replaced with —O—, —C(O)—, —C(S)—, —C(R)₂—, —CH(R)—, —CF(R)—, —C(F)₂—, —N(R)—, —S—, —S(O)₂— or —CR═CR—.

In some embodiments, L^{a} is a covalent bond. In some embodiments, L^{a} is a C₁₋₃ bivalent straight or branched saturated or unsaturated hydrocarbon chain wherein 1-3 methylene units of the chain are independently and optionally replaced with —O—, —C(O)—, —C(S)—, —C(R)₂—, —CH(R)—, —CF(R)—, —C(F)₂—, —N(R)—, —S—, —S(O)₂— or —CR═CR—.

In some embodiments, L^{a} is a covalent bond or a C₁₋₃ bivalent straight saturated hydrocarbon chain wherein 1-3 methylene units of the chain are independently and optionally replaced with —O—, —C(O)—, —C(S)—, —CH₂—, —CH(F)—, —C(F)₂—, —NH—, —NCH₃—, —S—, —S(O)₂— or —CR═CR—.

In some embodiments, L^{a} is covalent bond or a C₁₋₃ bivalent straight saturated hydrocarbon chain wherein 1-3 methylene units of the chain are independently and optionally replaced with —C(O)—, —C(S)—, —CH₂—, —CH(F)—, —C(F)₂—, —NH—, —NCH₃— or —C(O)NH—.

In some embodiments, L^{a} is covalent bond or a C₁₋₃ bivalent straight saturated hydrocarbon chain wherein 1-3 methylene units of the chain are independently and optionally replaced with —C(O)—, —NH—, or —C(O)NH—.

In some embodiments, L^{a} is a C₁₋₃ bivalent straight saturated hydrocarbon chain wherein 1-3 methylene units of the chain are independently and optionally replaced with —C(O)NH—.

In some embodiments, L^{a} is covalent bond, —C(O)—, —C(S)—, —CH₂—, —CH(F)—, —C(F)₂—, —NH—, —NCH₃— or —C(O)NH—.

In some embodiments, L^{a} is covalent bond, —C(O)—, —NH—, or —C(O)NH—.

In some embodiments, L^{a} is —C(O)NH—.

In some embodiments, L^{b} is a covalent bond. In some embodiments, L^{b} is a C₁₋₃ bivalent straight or branched saturated or unsaturated hydrocarbon chain wherein 1-3 methylene units of the chain are independently and optionally replaced with —O—, —C(O)—, —C(S)—, —C(R)₂—, —CH(R)—, —CF(R)—, —C(F)₂—, —N(R)—, —S—, —S(O)₂— or —CR═CR—.

In some embodiments, L^{b} is a covalent bond. In some embodiments, L^{a} is a C₁₋₃ bivalent straight or branched saturated or unsaturated hydrocarbon chain wherein 1-3 methylene units of the chain are independently and optionally replaced with —O—, —C(O)—, —C(S)—, —C(R)₂—, —CH(R)—, —CF(R)—, —C(F)₂—, —N(R)—, —S—, —S(O)₂— or —CR═CR—.

In some embodiments, L^{b} is a covalent bond or a C₁₋₃ bivalent straight saturated hydrocarbon chain wherein 1-3 methylene units of the chain are independently and optionally replaced with —O—, —C(O)—, —C(S)—, —CH₂—, —CH(F)—, —C(F)₂—, —NH—, —NCH₃—, —S—, —S(O)₂— or —CR═CR—.

In some embodiments, L^{b} is covalent bond or a C₁₋₃ bivalent straight saturated hydrocarbon chain wherein 1-3 methylene units of the chain are independently and optionally replaced with —C(O)—, —C(S)—., —CH₂—, —CH(F)—, —C(F)₂—, —NH—, —NCH₃— or —C(O)NH—.

In some embodiments, L^{b} is covalent bond or a C₁₋₃ bivalent straight saturated hydrocarbon chain wherein 1-3 methylene units of the chain are independently and optionally replaced with —C(O)—, —NH—, or —C(O)NH—.

In some embodiments, L^{b} is a C₁₋₃ bivalent straight saturated hydrocarbon chain wherein 1-3 methylene units of the chain are independently and optionally replaced with —C(O)NH—.

In some embodiments, L^{b} is covalent bond, —C(O)—, —C(S)—, —CH₂—, —CH(F)—, —C(F)₂—, —NH—, —NCH₃— or —C(O)NH—.

In some embodiments, L^{b} is covalent bond, —C(O)—, —NH—, or —C(O)NH—.

In some embodiments, L^{b} is —C(O)NH—.

In some embodiments, L^{a} and L^{b} are selected from those depicted in Table 1, below.

As defined above and described herein, a, b, and c are each independently 0, 1, 2, 3 or 4.

In some embodiments, a is 0. In some embodiments, a is 1. In some embodiments, a is 2. In some embodiments, a is 3. In some embodiments, a is 4. In some embodiments, a is 0 or 1. In some embodiments, a is 1 or 2. In some embodiments, a is 1, 2, or 3.

In some embodiments, b is 0. In some embodiments, b is 1. In some embodiments, b is 2. In some embodiments, b is 3. In some embodiments, b is 4. In some embodiments, b is 0 or 1. In some embodiments, b is 1 or 2. In some embodiments, b is 1, 2, or 3.

In some embodiments, c is 0. In some embodiments, c is 1. In some embodiments, c is 2. In some embodiments, e is 3. In some embodiments, c is 4. In some embodiments, c is 0 or 1. In some embodiments, c is 1 or 2. In some embodiments, c is 1, 2, or 3.

In some embodiments, a, b, and c are selected from those depicted in Table 1, below.

As defined above and described herein, e is 0 or 1.

In some embodiments, e is 0. In some embodiments, e is 1.

In some embodiments, e is selected from those depicted in Table 1, below.

As defined above and described herein, X is —O—, —N(R)—, or —S—.

In some embodiments, X is —O—. In some embodiments, X is —N(R)—. In some embodiments, X is —S—.

In some embodiments, X is —O—, —NH—, —NCH₃—, or —S—.

In some embodiments, X is —NH— or —NCH₃—.

In some embodiments, X is selected from those depicted in Table 1, below.

As defined above and described herein, Y is O, N(R), or S.

In some embodiments, Y is O. In some embodiments, Y is N(R). In some embodiments, Y is S.

In some embodiments, Y is O, NH, —N(C₁₋₆ alkyl), or S.

In some embodiments, Y is —NH, —N(C₁₋₆ alkyl).

In some embodiments, Y is selected from those depicted in Table 1, below.

In some embodiments, KBM is

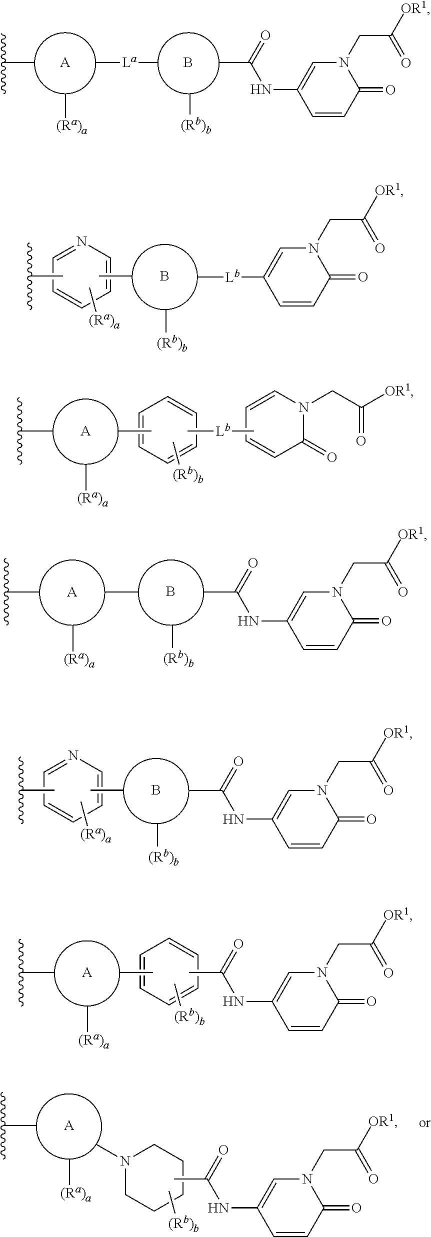

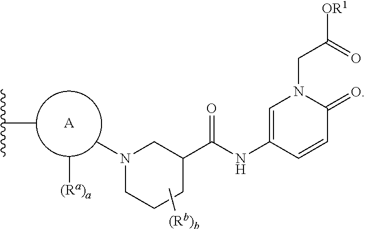

In some embodiments, KBM is

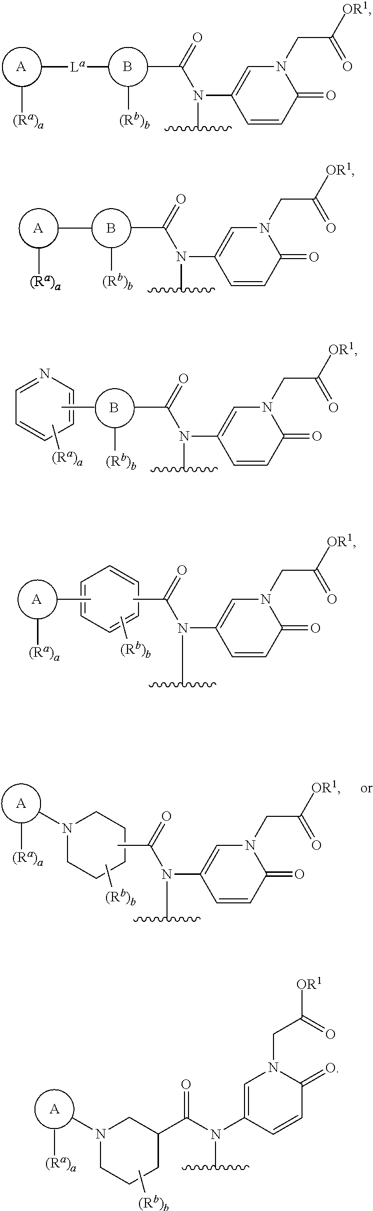

In some embodiments, KBM is

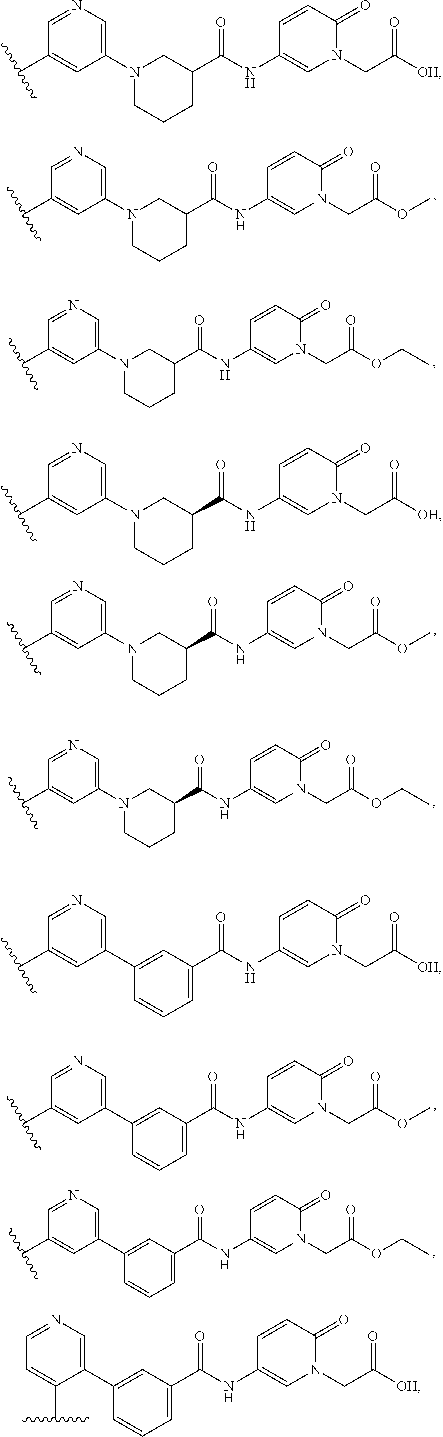

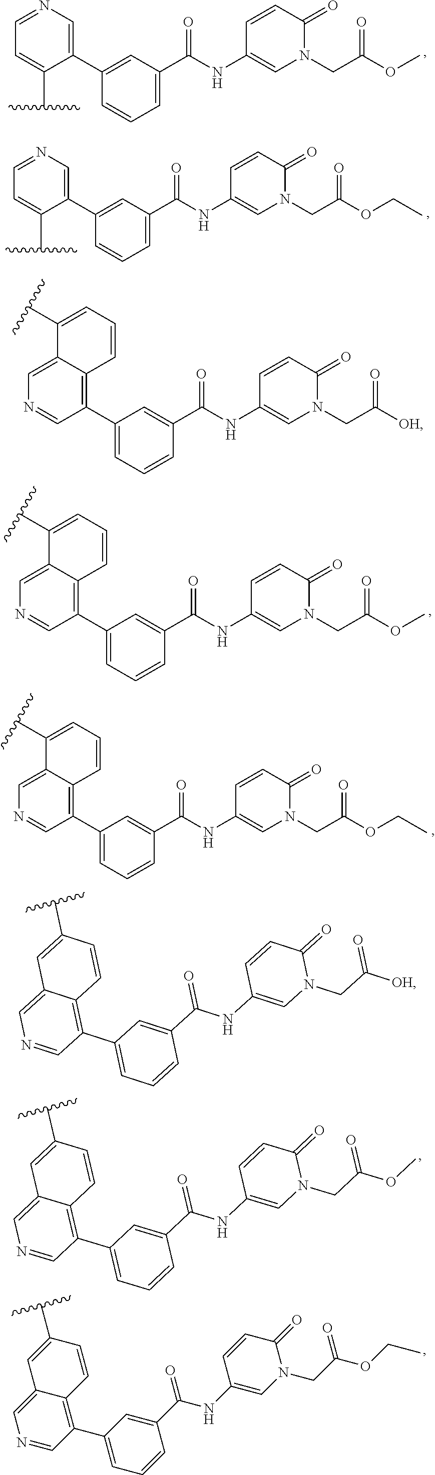

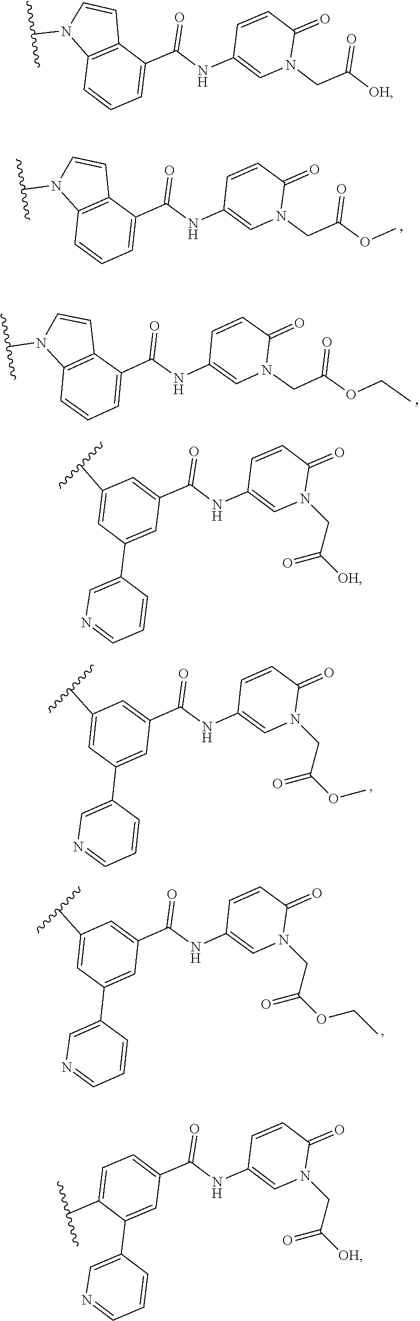

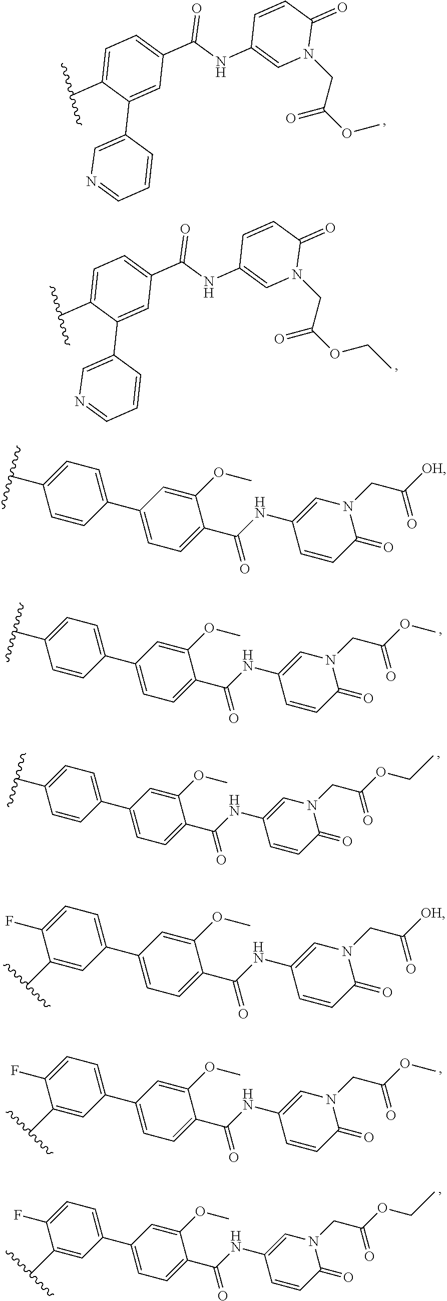

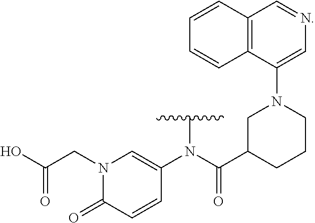 [SMILES: *N(C(=O)C1CCCN(c2cncc3ccccc23)C1)c1ccc(=O)n(CC(=O)O)c1]

In some embodiments, KBM is selected from those depicted in Table 1, below.

In certain embodiments, the present invention provides a compound of formula III-a represented by any one of the following formulae:

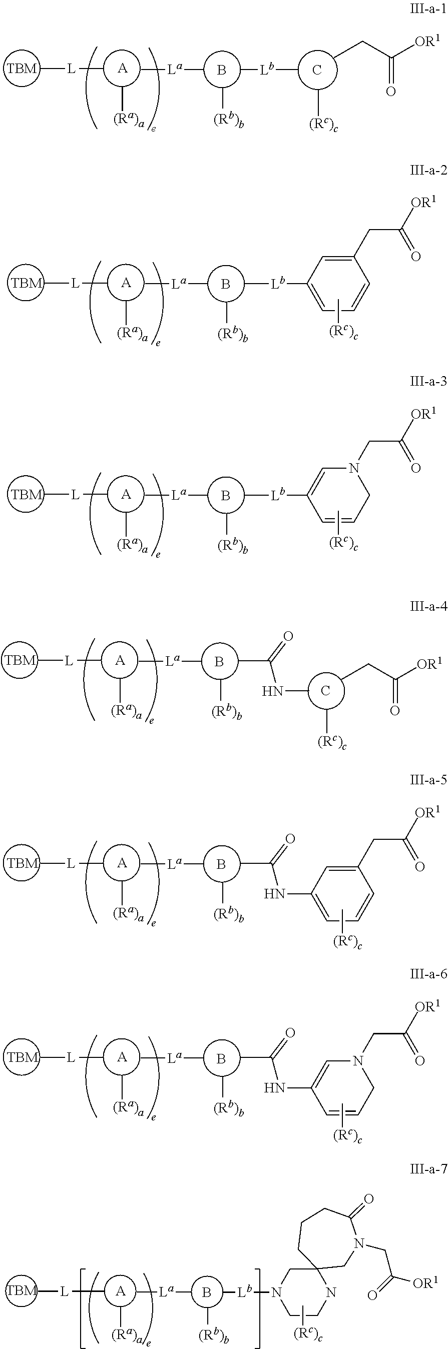

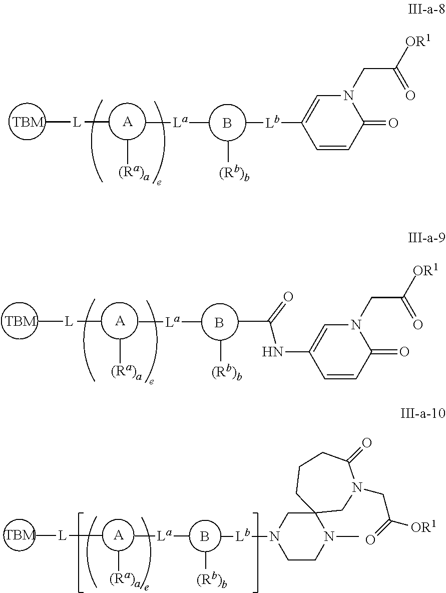

or a pharmaceutically acceptable salt thereof, wherein each of the variables is as defined and described herein, both independently and in combination.

In certain embodiments, the present invention provides a compound of formula IV:

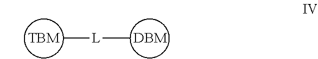

or a pharmaceutically acceptable salt thereof, wherein:
TBM is a TYK2 binding moiety capable of binding to TYK2 protein;
L is a bivalent moiety that connects TBM to DBM; and
DBM is DCAF E3 ubiquitin ligase binding moiety capable of binding to DCAF1 protein.

In some embodiments, the present invention provides a compound of formula IV, wherein TBM-L-is any one of the following:

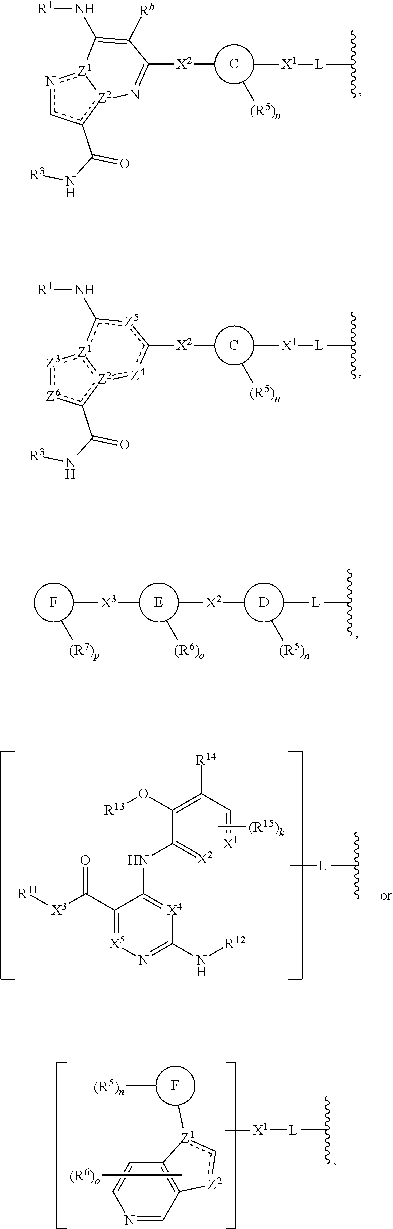

wherein each of the variables is as defined above in formula I-a, I-a′, I-a″, I-b, II-cc, and I-c or subgenera thereof and described in embodiments herein, both singly and in combination.

As described above, in certain embodiments, the present invention provides a compound of formula IV, wherein DBM is a compound of formula IV-a or IV-b:

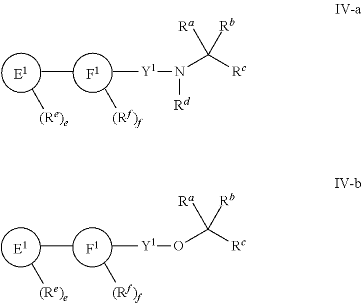

or a pharmaceutically acceptable salt thereof, wherein:
Ring E¹ is phenyl, naphthyl, a 4-9 membered partially unsaturated monocyclic, bicyclic, or bridged bicyclic carbocyclyl or heterocyclyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Ring F¹ is a 5-membered monocyclic heteroarylenyl with 1-3 heteroatoms independently selected from nitrogen, oxygen and sulfur.

Y¹ is a C₁₋₃ hydrocarbon chain wherein each methylene is optionally replaced with —CR₂—, —CR(OR)—, —C(O)—, —C(NR)—, —C(NOR)—, —S(O)—, or —S(O)₂—; or —C(OR)═ in formula IV-a where R^{d} is absent;

R^{a} is hydrogen, an optionally substituted C₁₋₆ aliphatic, or

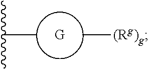

Ring G is phenyl, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur; R is hydrogen, an optionally substituted C₁₋₆ aliphatic, phenyl, or a 5-6 membered heteroaryl with 1-4 heteroatoms independently selected from nitrogen, oxygen and sulfur, or:
   R^{a} and R^{b} are taken together with their intervening atoms to form an optionally substituted 9-10 membered saturated or partially unsaturated bicyclic ring having 0-3 heteroatoms independently selected from nitrogen, oxygen, and sulfur, or:
   when Y¹ is —C(NR)—, R^{b} is taken together with R of —C(NR)— with their intervening atoms to form a 5-7 membered partially unsaturated heterocyclyl with 0-1 heteroatoms, in addition to the 2 heteroatoms within the heterocyclyl, independently selected from nitrogen, oxygen, and sulfur;
R^{c} is —CO₂R, —CONR₂, —CR₂CF₂R, —CR₂CONR₂, —CR₂C(O)R, —CR₂CO₂R, —CR₂NR₂, —CR₂OR, —CR₂SO₂NR₂, —CR₂S(O)R, —CR₂SO₂R, —CR₂S(O)(NR)R, —CR₂CN. —CR²CR₂NR₂, —CR₂CR₂OR, —CR₂CR═NOR, —CR₂CR(OR)CR₂OR, or an optionally substituted group selected from phenyl; a 4-7 membered saturated or partially unsaturated carbocyclyl or heterocyclyl with 1-3 heteroatoms independently selected from nitrogen, oxygen, and sulfur; a 5-9 membered monocyclic or bicyclic heteroaryl with 1-4 heteroatoms independently selected from nitrogen, oxygen and sulfur; or:
   —(CR₂)₁₋₂—X^{a}, wherein X^{a} is halogen or an optionally substituted ring selected from phenyl; a 4-7 membered saturated or partially unsaturated carbocyclyl or heterocyclyl with 1-3 heteroatoms independently selected from nitrogen, oxygen, and sulfur; and a 5-9 membered monocyclic or bicyclic heteroaryl with 1-4 heteroatoms independently selected from nitrogen, oxygen and sulfur; or: R^{b} and R^{c} are taken together with their intervening atoms to form an optionally substituted 4-6 membered saturated or partially unsaturated carbocyclyl or heterocyclyl with 1-3 heteroatoms independently selected from nitrogen, oxygen, and sulfur; or:
   R^{a} is absent and R^{b} and R^{c} are taken together with their intervening atoms to form an optionally substituted phenyl; or:
   when Y¹ is —C(OR)═, R^{c} is taken together with R of —C(OR)═ with their intervening atoms to form a 5-7 membered partially unsaturated heterocyclyl with 0-1 heteroatoms, in addition to the 2 heteroatoms within the heterocyclyl, independently selected from nitrogen, oxygen, and sulfur;
R^{d} is hydrogen or an optionally substituted C₁₋₆ aliphatic, or:
   when R^{c} is —CR₂CONR₂, R^{d} is taken together with a single R of —CR₂CONR₂ with their intervening atoms to form a 5-7 membered saturated or partially unsaturated heterocyclyl with 0-3 heteroatoms, in addition to the nitrogen atom to which R^{d} is attached, independently selected from nitrogen, oxygen, and sulfur;
R^{e}, R^{f}, and R^{g} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —C(NOR)R, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, naphthyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form an optionally substituted 3-7 membered saturated or partially unsaturated ring having 0-3 heteroatoms, in addition to the atom to which they are attached, independently selected from nitrogen, oxygen, and sulfur; and
each of e, f, and g are independently 0, 1, 2, 3, or 4.

As described above, in certain embodiments, the present invention provides a compound of formula IV, wherein DBM is a compound of formula IV-c:

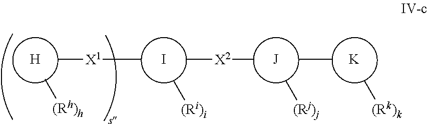

or a pharmaceutically acceptable salt thereof, wherein:
Ring H is a 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
Ring I is phenylenyl, a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur, or a 5-10 membered monocyclic or bicyclic heteroarylenyl with 1-4 heteroatoms independently selected from nitrogen, oxygen and sulfur;
Ring J is phenylenyl, a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur, or a 5-10 membered monocyclic or bicyclic heteroaryl ring having 1-4 heteroatoms independently selected from nitrogen, oxygen, and sulfur;
Ring K is phenyl, naphthyl, a 3-7 membered saturated or partially unsaturated carbocyclylenyl or heterocyclylenyl with 1-3 heteroatoms independently selected from nitrogen, oxygen, and sulfur, a 9-10 membered saturated or partially unsaturated bicyclic heterocyclylenyl with 1-3 heteroatoms independently selected from nitrogen, oxygen, and sulfur, or a 5-13 membered monocyclic, bicyclic, or tricyclic heteroarylenyl with 1-5 heteroatoms independently selected from nitrogen, oxygen and sulfur;
R^{h}, Rⁱ, R^{j}, and R^{k} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂, or:
   an R¹ group on Ring I and an R^{J} group or Ring J are optionally taken together with their intervening atoms to form a 5-8 membered saturated, partially unsaturated, or aromatic ring having 0-3 heteroatoms independently selected from nitrogen, oxygen, and sulfur, or:
   an R group on Ring J and an R group or Ring K are optionally taken together with their intervening atoms to form a 5-6 membered partially unsaturated or aromatic ring having 0-3 heteroatoms independently selected from nitrogen, oxygen, and sulfur;
each R is independently hydrogen, or an optionally substituted group selected from C₁₋₆ aliphatic, phenyl, naphthyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-7 membered saturated or partially unsaturated ring having 0-3 heteroatoms, in addition to the atom to which they are attached, independently selected from nitrogen, oxygen, and sulfur;
each of X¹ and X² are independently a covalent bond, spiro-fusion between the two rings that X¹ or X² connect, or a bivalent, saturated or unsaturated, straight or branched C₁₋₆ hydrocarbon chain, wherein 0-4 methylene units of X¹ and X² are independently replaced by —CR₂—, —CR(OR)—, —CRF—, —CF₂—, —C(NR)—, —C(O)—, —O—, —N(R)—, —S—, —S(O)—, or —S(O)₂—;
s″ is 0 or 1; and
each of w, x, y, and z are independently 0, 1, 2, 3, or 4.

As described above and defined herein, Ring E¹ is phenyl, naphthyl, a 4-9 membered partially unsaturated monocyclic, bicyclic, or bridged bicyclic carbocyclyl or heterocyclyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur.

In some embodiments, Ring E¹ is phenyl. In some embodiments, Ring E¹ is naphthyl. In some embodiments, Ring E¹ is a 4-9 membered partially unsaturated monocyclic, bicyclic, or bridged bicyclic carbocyclyl. In some embodiments, Ring E^{L} is a 4-9 membered partially unsaturated monocyclic, bicyclic, or bridged bicyclic heterocyclyl with 1-3 heteroatoms independently selected from nitrogen, oxygen, and sulfur. In some embodiments, Ring E¹ is a 5-9 membered monocyclic or bicyclic heteroarylenyl with 1-4 heteroatoms independently selected from nitrogen, oxygen and sulfur.

In some embodiments, Ring E¹ is cyclobutyl, azetinyl, cyclohexyl, cyclohexenyl, tetrahydro-2H-pyranyl, 3,6-dihydro-2H-pyranyl, pyrrolidinyl, imidazolyl, 4,5-dihydro-1H-pyrazolyl, piperidinyl, phenyl, isoxazolyl, isothiazolyl, pyrazolyl, pyridyl, pyridazinyl, pyrimidinyl, 2,3-dihydro-1H-indenyl, indolyl, benzoimidazolyl, pyrazolo[1,5-a]pyridyl, [1,2,4]triazolo[1,5-a]pyridyl, bicyclo[2.2.2]octane, or naphthyl.

In some embodiments, Ring E¹ is as depicted in the compounds of Table 1, below.

In some embodiments, Ring F¹ is cyclobutylenyl, azetinylenyl, cyclopentylenyl cyclohexylenyl, phenylenyl, pyrrolylenyl, imidazolylenyl, pyrazolylenyl, isoxazolylenyl, thiophenylenyl, 2,5-dihydro-1H-pyrrolylenyl, 1,2,3-triazolylenyl, 1,2,4-triazolylenyl, 1,2-dihydro-3H-pyrazol-3-onylenyl, thiazolvlenyl, pyridvlenyl, indazolylenyl, 1,2,3,6-tetrahydropyridinylenyl, benzoimidazolylenyl, 3,4-dihydroquinolinylenyl, 4,5,6,7-tetrahydro-1H-pyrazolo[4,3-b]pyridylenyl, 2,3,4,5-tetrahydrobenzo[f][1,4]oxazepanylenyl, 4,5,6,7-tetrahydropyrazolo[1,5-a]pyrimidinylenyl, 1,3,4,5-tetrahydro-2H-benzo[b][1,4]diazepin-2-onylenyl, or 4,5,6,7-tetrahydro-1H-pyrazolo[4,3-c]pyridylenyl.

In some embodiments, Ring F¹ is as depicted in the compounds of Table 1, below.

As described above and defined herein, Ring G is phenyl, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur.

In some embodiments, Ring G is phenyl. In some embodiments, Ring G is a 5-7 membered saturated or partially unsaturated carbocyclyl. In some embodiments, Ring G is a 5-7 membered saturated or partially unsaturated heterocyclyl with 1-3 heteroatoms independently selected from nitrogen, oxygen, and sulfur. In some embodiments, Ring G is a 5-6 membered heteroaryl with 1-4 heteroatoms independently selected from nitrogen, oxygen and sulfur.

In some embodiments, Ring G is cyclohexyl, cyclohexenyl, isothiazolyl, phenyl, or pyridyl.

In some embodiments, Ring G is as depicted in the compounds of Table 1, below.

As described above and defined herein, Ring H is a 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur.

In some embodiments, Ring H is a 3-11 membered saturated or partially unsaturated monocyclic, bicyclic, bridged bicyclic, or spirocyclic carbocyclyl. In some embodiments, Ring H is a 3-11 membered saturated or partially unsaturated monocyclic, bicyclic, bridged bicyclic, or spirocyclic heterocyclyl with 1-3 heteroatoms independently selected from nitrogen, oxygen, and sulfur.

In some embodiments, Ring H is cyclopropyl, cyclobutyl, azetinyl, cyclopentyl, pyrrolidinyl, cyclohexyl, piperidinyl, piperazinyl, 3,6-dihydro-2H-pyranyl, tetrahydro-2H-pyranyl, morpholinyl, piperzinyl, isoindolinyl, 3-azabicyclo[3.1.0]hexanyl, 3-azabicyclo[3.1.1]heptanyl, 6-azabicyclo[3.1.1]heptanyl, azepanyl, 2-3-azabicyclo[3.2.2]nonanyl, azaspiro[3.3]heptanyl, 5-azaspiro[2.5]octanyl, 2,7-diazaspiro[3.5]nonanyl, 3-azaspiro[5.5]undecanyl, 2-azabicyclo[3.2.1]octanyl, 3-azabicyclo[3.2.1]octanyl, 8-azabicyclo[3.2.1]octanyl, octahydrocyclopenta[c]pyrrolyl, 3,4-dihydro-2H-pyrido[3,2-b][1,4]oxazinyl, 2-oxa-5-azabicyclo[2.2.1]heptanyl, 6-oxa-3-azabicyclo[3.1.1]heptanyl, or 2-oxa-5-azabicyclo[2.2.2]octanyl.

In some embodiments, Ring H is as depicted in the compounds of Table 1, below.

As described above and defined herein, Ring I is phenylenyl, a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur, or a 5-10 membered monocyclic or bicyclic heteroarylenyl with 1-4 heteroatoms independently selected from nitrogen, oxygen and sulfur.

In some embodiments, Ring I is phenylenyl. In some embodiments, Ring I is a 3-11 membered saturated or partially unsaturated monocyclic, bicyclic, bridged bicyclic, or spirocyclic carbocyclylenyl. In some embodiments, Ring I is a 3-11 membered saturated or partially unsaturated monocyclic, bicyclic, bridged bicyclic, or spirocyclic heterocyclylenyl with 1-3 heteroatoms independently selected from nitrogen, oxygen, and sulfur. In some embodiments, Ring I is a 5-10 membered monocyclic or bicyclic heteroarylenyl with 1-4 heteroatoms independently selected from nitrogen, oxygen and sulfur.

In some embodiments, Ring I is cyclohexylenyl, phenylenyl, imidazolylenyl, pyrazolylenyl, oxazolylenyl, thiazolylenyl, 1,2-thiazinanylenyl, pyridylenyl, pyridazinylenyl, pyrimidinylenyl, indolinylenyl, 2,6-diazaspiro[3.5]nonanylenyl, 2,3-dihydro-1H-pyrrolo[2,3-b]pyridylenyl, 2,3-dihydro-1H-pyrrolo[3,2-c]pyridylenyl, 1H-pyrrolo[2,3-b]pyridylenyl, 7H-pyrrolo[2,3-d]pyrimidinylenyl, 111-imidazo[4,5-b]pyridinylenyl, 3H-imidazo[4,5-b]pyridylenyl, 9H-purinylenyl, octahydro-1H-pyrrolo[3,2-b]pyridinylenyl, decahydroisoquinolinylenyl, quinolinylenyl, isoquinolinylenyl, 3,4-dihydroquinoxalin-2(1H)-onylenyl, 1,2,3,4-tetrahydroquinoxalinylenyl, 1,2,3,4-tetrahydropyrido[2,3-b]pyrazinylenyl, 1,2,3,4-tetrahydro-1,8-naphthyridinylenyl, or 1,2,3,4-tetrahydro-1,6-naphthyridinylenyl.

In some embodiments, Ring I is as depicted in the compounds of Table 1, below.

As described above and defined herein, Ring J is phenylenyl, a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur, or a 5-6 membered heteroaryl ring having 1-4 heteroatoms independently selected from nitrogen, oxygen, and sulfur.

In some embodiments, Ring J is phenylenyl. In some embodiments, Ring J is a 3-11 membered saturated or partially unsaturated monocyclic, bicyclic, bridged bicyclic, or spirocyclic carbocyclyl. In some embodiments, Ring J is a 3-11 membered saturated or partially unsaturated monocyclic, bicyclic, bridged bicyclic, or spirocyclic heterocyclyl with 1-3 heteroatoms independently selected from nitrogen, oxygen, and sulfur. In some embodiments, Ring J is a 5-10 membered monocyclic or bicyclic heteroaryl ring having 1-4 heteroatoms independently selected from nitrogen, oxygen, and sulfur.

In some embodiments, Ring J is cyclohexylenyl, azetidinylenyl, pyrrolidinylenyl, imidazolylenyl, thiazolylenyl, piperidinylenyl, piperzinylenyl, azepanylenyl, phenylenyl, pyridinylenyl, isoindolinyl, quinazolinylenyl, octahydro-1H-indolylenyl, 2,3-dihydro-1H-pyrrolo[2,3-b]pyridvlenyl, 8-azabicyclo[3.2.1]octanylenyl, 2-azabicyclo[3.2.1]octanylenyl, 2,7-diazaspiro[4.4]nonanylenyl, octahydropyrrolo[3,2-b]pyrrolylenyl, 2-azabicyclo[3.2.2]nonanylenyl, octahydro-1H-pyrrolo[3,2-b]pyridylenyl, octahydro-1H-pyrrolo[3,4-b]pyridinylenyl, decahydro-1,5-naphthyridinylenyl, 9-azabicyclo[3.3.1]nonanylenyl, 5-azaspiro[3.5]nonanylenyl, 2-oxa-5-azaspiro[3.5]nonanylenyl, or 2,6-diazaspiro[3.5]nonanylenyl.

In some embodiments, Ring J is as depicted in the compounds of Table 1, below.

As described above and defined herein, Ring K is phenyl, naphthyl, a 3-7 membered saturated or partially unsaturated carbocyclyl or heterocyclyl with 1-3 heteroatoms independently selected from nitrogen, oxygen, and sulfur, a 9-10 membered saturated or partially unsaturated bicyclic heterocyclyl with 1-3 heteroatoms independently selected from nitrogen, oxygen, and sulfur, or a 5-13 membered monocyclic, bicyclic, or tricyclic heteroaryl with 1-5 heteroatoms independently selected from nitrogen, oxygen and sulfur.

In some embodiments, Ring K is phenyl. In some embodiments, Ring K is naphthyl. In some embodiments, Ring K is a 3-7 membered saturated or partially unsaturated carbocyclyl. In some embodiments, Ring K is a 3-7 membered saturated or partially unsaturated heterocyclyl with 1-3 heteroatoms independently selected from nitrogen, oxygen, and sulfur. In some embodiments, Ring K is a 9-10 membered saturated or partially unsaturated bicyclic heterocyclyl with 1-3 heteroatoms independently selected from nitrogen, oxygen, and sulfur. In some embodiments, Ring K is a 5-13 membered monocyclic, bicyclic, or tricyclic heteroaryl with 1-5 heteroatoms independently selected from nitrogen, oxygen and sulfur.

In some embodiments, Ring K is 1,2,3-triazolyl, thiazolyl, pyrazolyl, cyclohexyl, piperdinyl, phenyl, pyridinyl, pyridazinyl, pyrimidinyl, 2,3-dihydro-1H-indenyl, purinyl, indazolyl, benzo[d]imidazoyl, benzo[d][1,3]dioxolyl, benzo[b]thiophenyl, benzo[d]isoxazolyl, benzo[d]isothiazolyl, pyrrolo[3,2-c]pyridinyl, imidazo[1,2-a]pyridinyl, imidazo[1,2-b]pyridazinyl, pyrazolo[1,5-a]pyrimidinyl, pyrrolo[2,3-d]pyrimidinyl, 6,7-dihydro-5H-cyclopenta[d]pyrimidinyl, 2,3-dihydro-1H-pyrrolo[2,3-c]pyridinyl, 6,7-dihydro-5H-cyclopenta[b]pyridinyl, 2,3-dihydro-1H-pyrrolo[3,2-c]pyridinyl, 1,2,3,4-tetrahydroquinolinyl, 1,2,3,4-tetrahydroisoquinolinyl, naphthyl, 5,6,7,8-tetrahydropyrido[3,4-d]pyrimidinyl, 5,6,7,8-tetrahydropyrido[4,3-d]pyrimidinyl, quinolinyl, isoquinolinyl, isoquinolin-3(2H)-onyl, 1,6-naphthyridinyl, phthalazinyl, quinazolinyl, 2,7-naphthyridinyl, or tetrazolo[1,5-a]quinoxalinyl.

In some embodiments, Ring K is as depicted in the compounds of Table 1, below.

As described above and defined herein, R^{a} is hydrogen, an optionally substituted C₁₋₆ aliphatic or

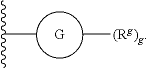

In some embodiments, R^{a} is hydrogen. In some embodiments, R^{a} is an optionally substituted C₁₋₆ aliphatic. In some embodiments, R^{a} is

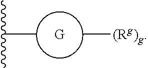

In some embodiments, Ring R^{a} is methyl, —CH(Me)OH, benzyl, —CH₂tolyl, or —CH₂indolyl.

As described above and defined herein, R^{b} is hydrogen, an optionally substituted C₁₋₆ aliphatic, phenyl, or a 5-6 membered heteroaryl with 1-4 heteroatoms independently selected from nitrogen, oxygen and sulfur, or R^{a} and R^{b} are optionally taken together with their intervening atoms to form an optionally substituted 9-10 membered saturated or partially unsaturated bicyclic ring having 0-3 heteroatoms independently selected from nitrogen, oxygen, and sulfur, or when Y is —C(NR)—, R^{b} is optionally taken together with R of —C(NR)— with their intervening atoms to form a 5-7 membered partially unsaturated heterocyclyl with 0-1 heteroatoms, in addition to the 2 nitrogen atoms within the heterocyclyl, independently selected from nitrogen, oxygen, and sulfur.

In some embodiments, R^{b} is hydrogen. In some embodiments, R^{b} is hydrogen is an optionally substituted C₁₋₆ aliphatic. In some embodiments, R^{b} is hydrogen is phenyl. In some embodiments, R^{b} is hydrogen is a 5-6 membered heteroaryl with 1-4 heteroatoms independently selected from nitrogen, oxygen and sulfur. In some embodiments, R^{a} and R^{b} are optionally taken together with their intervening atoms to form an optionally substituted 9-10 membered saturated or partially unsaturated bicyclic ring having 0-3 heteroatoms independently selected from nitrogen, oxygen, and sulfur. In some embodiments, when Y is —C(NR)—, R^{b} is optionally taken together with R of —C(NR)— with their intervening atoms to form a 5-7 membered partially unsaturated heterocyclyl with 0-1 heteroatoms, in addition to the 2 nitrogen atoms within the heterocyclyl, independently selected from nitrogen, oxygen, and sulfur.

In some embodiment, R^{b} is methyl, —CH(Me)OH, cyclopropyl, phenyl, —CO₂H, -CH₂cyclopropyl, —CH₂OH, —CH₂OMe, or —CH₂CO₂H.

As described above and defined herein, R^{b} is —CO₂R, —CONR₂, —CR₂CF₂R, —CR₂CONR₂, —CR₂C(O)R, —CR₂CO₂R, —CR₂NR₂, —CR₂OR—CR₂SO₂NR₂, —CR₂S(O)R, —CR₂SO₂R, —CR₂S(O)(NR)R, —CR₂CN, —CR₂CR₂NR₂, —CR₂CR₂OR, —CR₂CR═NOR, —CR₂CR(OR)CR₂OR, or an optionally substituted group selected from phenyl; a 4-7 membered saturated or partially unsaturated carbocyclyl or heterocyclyl with 1-3 heteroatoms independently selected from nitrogen, oxygen, and sulfur: a 5-9 membered monocyclic or bicyclic heteroaryl with 1-4 heteroatoms independently selected from nitrogen, oxygen and sulfur; or: —(CR₂)₁₋₂—X^{a}, wherein X^{a} is halogen or an optionally substituted ring selected from phenyl; a 4-7 membered saturated or partially unsaturated carbocyclyl or heterocyclyl with 1-3 heteroatoms independently selected from nitrogen, oxygen, and sulfur; and a 5-9 membered monocyclic or bicyclic heteroaryl with 1-4 heteroatoms independently selected from nitrogen, oxygen and sulfur; or: R^{b} and R^{c} are taken together with their intervening atoms to form an optionally substituted 4-6 membered saturated or partially unsaturated carbocyclyl or heterocyclyl with 1-3 heteroatoms independently selected from nitrogen, oxygen, and sulfur; or: R^{a} is absent and R^{b} and R^{c} are taken together with their intervening atoms to form an optionally substituted phenyl; or: when Y¹ is —C(OR)═, R^{c} is taken together with R of —C(OR)═ with their intervening atoms to form a 5-7 membered partially unsaturated heterocyclyl with 0-1 heteroatoms, in addition to the 2 heteroatoms within the heterocyclyl, independently selected from nitrogen, oxygen, and sulfur.

In some embodiments, R^{c} is —CO₂R. In some embodiments, R^{c} is —CONR₂. In some embodiments, R^{c} is —CR₂CF₂R. In some embodiments, R^{c} is —CR₂CONR₂. In some embodiments, R^{c} is —CR₂C(O)R. In some embodiments, R^{c} is —CR₂CO₂R. In some embodiments, R¹ is —CR₂NR₂. In some embodiments, R^{c} is —CR₂OH. In some embodiments, R^{c} is —CR₂SO₂NR₂. In some embodiments, R^{c} is —CR₂S(O)R. In some embodiments, R^{c} is —CR₂SO₂R. In some embodiments, R^{c} is —CR₂S(O)(NR)R. In some embodiments, R^{c} is —CR₂CN. In some embodiments, R^{c} is —CR₂CR₂NR₂. In some embodiments, R^{c} is —CR₂CR₂OR. In some embodiments, R^{c} is —CR₂CR═NOR. In some embodiments, R^{c} is —CR₂CR(OR)CR₂OR. In some embodiments, R^{c} is an optionally substituted phenyl. In some embodiments, R^{c} is an optionally substituted 4-7 membered saturated or partially unsaturated carbocyclyl. In some embodiments, R^{c} is an optionally substituted 4-7 membered saturated or partially unsaturated heterocyclyl with 1-3 heteroatoms independently selected from nitrogen, oxygen, and sulfur. In some embodiments, R^{c} is an optionally substituted 5-9 membered monocyclic or bicyclic heteroaryl with 1-4 heteroatoms independently selected from nitrogen, oxygen and sulfur.

In some embodiments, R^{c} is —(CR₂)₁₋₂—X³, wherein X^{a} is halogen. In some embodiments, R^{c} is —(CR₂)₁₋₂—X^{a}, wherein X^{a} is an optionally substituted phenyl. In some embodiments, R^{c} is —(CR₂)-2-X^{a}, wherein X^{a} is an optionally substituted 4-7 membered saturated or partially unsaturated carbocyclyl. In some embodiments, R^{c} is —(CR₂)₁₋₂—X^{a}, wherein X^{a} is an optionally substituted 4-7 membered saturated or partially unsaturated heterocyclyl with 1-3 heteroatoms independently selected from nitrogen, oxygen, and sulfur. In some embodiments, R^{c} is —(CR₂)₁₋₂—X^{a}, wherein X^{a} is an optionally substituted 5-9 membered monocyclic or bicyclic heteroaryl with 1-4 heteroatoms independently selected from nitrogen, oxygen and sulfur.

In some embodiments, R^{c} is —CO₂Bn, —CONHPh, —CR₂CF₂Me, —CH₂CONH₂, —CH(Me)CONH₂, —CH₂CONHMe, —CH₂CONHEt, —CH₂COMe, —CH₂CON(Me)CH₂CH₂NH₂, —CH₂CONHCONH₂, —CH₂CONHCH₂Ph, -CH₂CONHcyclopropyl, —CH₂OH, —CH₂Cl, —CH₂NMe₂. —CH₂CO₂H, —CH₂CO₂Me, —CH₂COCH₂CH₂OMe, —CH₂COCH₂CH₂CH₂OMe, —CH(Ph)OH, —CH(PhF)OH, —CH(Ph)NH₂, —CH(PhMe)NH₂, —CH₂SOMe, —CH₂SO₂NHEt, —CH₂SO₂NHMe, —CH₂SO₂Me, —CH₂SO₂Et, —CH₂SO₂iPr, —CH₂S(O)(NH)Me, —CH₂S(O)(NMe)Me, —CH₂C(Me)=NOH, —CH₂C(Me)=NOMe, oxetanyl, thietanyl, theitanyl dioxide, pyrrolidin-2-onyl, piperidin-2-onyl, isoxazolyl, imidazolyl, pyrrolyl, tetrahydrofuranyl, tetrahydrothiophenyl dioxide, pyridine-2-onyl, phenyl, tolyl, indolyl, —CH₂CN, —CH(Me)CN, —CH₂CH₂Cl, —CH₂CH₂NH₂, —CH₂CH₂NMe₂, —CH₂CH₂OH, —CH₂CH₂OMe, —CH₂pyrrolyl, —CH₂pyridyl, —CH₂indolyl,

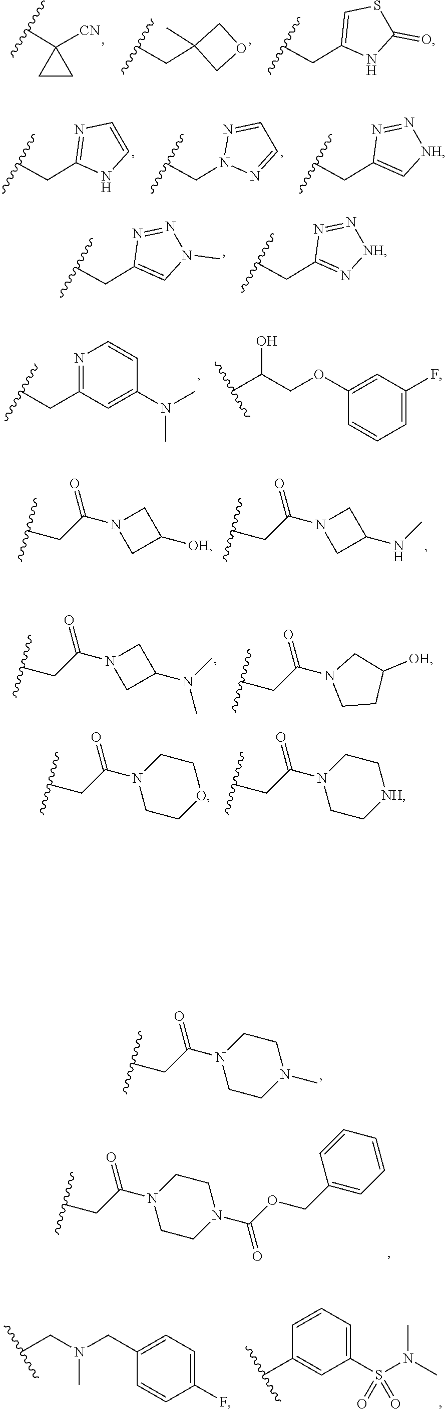

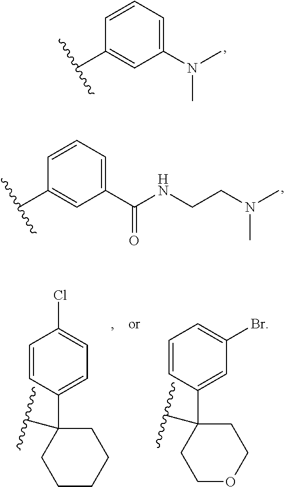

In some embodiments, R^{c} is as depicted in the compounds of Table 1, below.

As described above and defined herein, each of each of X¹ and X² are independently a covalent bond, spiro-fusion between the two rings that X¹ or X² connect, or a bivalent, saturated or unsaturated, straight or branched C₁₋₆ hydrocarbon chain, wherein 0-4 methylene units of X¹ and X² are independently replaced by —CR₂—, —CR(OR)—, —CRF—, —CF₂—, —C(NR)—, —C(O)—, —O—, —N(R)—, —S—, —S(O)—, or —S(O)₂—.

In some embodiments, X¹ is a covalent bond. In some embodiments, X¹ is a bivalent, saturated or unsaturated, straight or branched C₁₋₆ hydrocarbon chain, wherein 0-4 methylene units of X¹ and X² are independently replaced by —CR₂—, —CR(OR)—, —CRF—, —CF₂—, —C(NR)—, —C(O)—, —O—, —N(R)—, —S—, —S(O)—, or —S(O)₂—. In some embodiments, X¹ is —CR₂—. In some embodiments, X¹ is —CR(OR)—. In some embodiments, X¹—CRF—. In some embodiments, X¹ is —CF₂—. In some embodiments, X¹ is —(CR₂)₀₋₂—C(O)—. In some embodiments, X¹ is —CR₂NRCR₂—. In some embodiments, X¹ is —NRCR₂—. In some embodiments, X¹ is —C(O)NR—. In some embodiments, X¹ is —C(NR)NR—. In some embodiments, X¹ is —C(S)NR—. In some embodiments, X¹ is —NR—. In some embodiments, X¹ is —O—. In some embodiments, X¹ is —S—. In some embodiments, X¹ is —S(O)₂—. In some embodiments, X¹ represents spiro-fusion between the two rings that X¹ connect.

In some embodiments, X¹ is a covalent bond, —NH—, —NMe-.

In some embodiments, X² is a covalent bond. In some embodiments, X² is a bivalent, saturated or unsaturated, straight or branched C₁₋₆ hydrocarbon chain, wherein 0-4 methylene units of X¹ and X² are independently replaced by —CR₂—, —CR(OR)—, —CRF—, —CF₂—, —C(NR)—, —C(O)—, —O—, —N(R)—, —S—, —S(O)—, or —S(O)₂—. In some embodiments, X² is —CR₂—. In some embodiments, X² is —CR(OR)—. In some embodiments, X²—CRF—. In some embodiments, X² is —CF₂—. In some embodiments, X² is —(CR₂)₀₋₂—C(O)—. In some embodiments, X² is —CR₂NRCR₂—. In some embodiments, X² is —NRCR₂—. In some embodiments, X² is —C(O)NR—. In some embodiments, X² is —C(NR)NR—. In some embodiments, X² is —C(S)NR—. In some embodiments, X² is —NR—. In some embodiments, X² is —O—. In some embodiments, X² is —S—. In some embodiments, X² is —S(O)₂—. In some embodiments, X² represents spiro-fusion between the two rings that X² connects, e.g.,

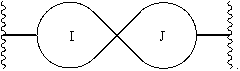

In some embodiments, X² is a covalent bond, —CH₂—, —CMe(OMe)-, —CMe(F)—, —CMe(CF₃)—, cyclopropylenyl, difluorocyclopropylenyl, —C(O)—, —CH₂CH₂C(O)—, —CH₂NHCH(Me)-, —NHCH₂—, —N(Me)CH₂—, —C(O)NH—, —NH—, —NMe-, —N(COMe)-, —N(CF₃)—, -NEt-, —N(nPr)-, —N(nBu)-, —N(Ph)-, —N(3-pyridyl)-, —N(4-pyridyl)-, —N(SO₂Me)-, —N(CH₂CHF₂)—, —N(CH₂cyclopropyl)-, —N(CH₂Ph)-, —N(CH₂CONH₂)—, —N(CH₂SO₂Me)-, —N(CH₂CH₂CHF₂)—, —N(CH₂CH₂Ph)-, —N(CH₂CH₂CO₂H)—, —N(CH₂CH₂CONH₂)—, —N(CH₂CH₂CN)—, —N(CH₂CH₂OMe)-, —N(CH₂CH₂SO₂Me)-, —O—, —S—, or —S(O)₂—.

In some embodiment, X¹ and X² are as depicted in the compounds of Table 1, below.

As described above and defined herein, Y¹ is a C-3 hydrocarbon chain wherein each methylene is optionally substituted with —CR₂—, —CR(OR)—, —C(O)—, —C(NR)—, —C(NOR)—, —S(O)—, or —S(O)₂—.

In some embodiments, Y¹ is a C₁₋₃ hydrocarbon chain wherein each methylene is optionally substituted with —CR₂—, —CR(OR)—, —C(O)—, —C(NR)—, —C(NOR)—, —S(O)—, or —S(O)₂—; or —C(OR)═ in formula IV-a where R^{d} is absent.

In some embodiments, Y¹ is a C₁₋₃ hydrocarbon chain. In some embodiments, Y¹ is —CR₂—. In some embodiments, Y¹ is —CR(OR)—. In some embodiments, Y¹ is —C(O)—. In some embodiments, Y¹ is —C(NR)—. In some embodiments, Y¹ is —C(NOR)—. In some embodiments, Y¹ is —S(O)—. In some embodiments, Y¹ is —S(O)₂—. In some embodiments, Y¹ is —C(OR)═ in formula IV-a where R^{d} is absent.

In some embodiments, Y¹ is —CH₂—, —CH(Me)-, —CH₂C(O)—, —NHCH₂C(O)—, —CH₂CH₂C(O)—, —CH₂CH(OH)C(O)—, —C(O)—, —C(NH)—, —C(NOH)—, —S(O)—, or —S(O)₂—.

As described above and defined herein, each of s′ and s″ are independently 0 or 1.

In some embodiments, s′ is 0. In some embodiments, s′ is 1. In some embodiments, s″ is 0. In some embodiments, s″ is 1.

As described above and defined herein, each of e, f, g, h, I, j, and k are independently 0, 1, 2, 3, or 4.

In some embodiments, e is 0. In some embodiments, e is 1. In some embodiments, e is 2. In some embodiments, e is 3. In some embodiments, e is 4.

In some embodiments, e is 0 or 1. In some embodiments, e is 1 or 2. In some embodiments, e is 2 or 3. In some embodiments, e is 3 or 4.

In some embodiments, f is 0, In some embodiments, f is 1. In some embodiments, f is 2. In some embodiments, f is 3. In some embodiments, f is 4.

In some embodiments, f is 0 or 1. In some embodiments, f is 1 or 2.

In some embodiments, g is 0. In some embodiments, g is 1. In some embodiments, g is 2. In some embodiments, g is 3. In some embodiments, g is 4.

In some embodiments, g is 0 or 1. In some embodiments, g is 1 or 2.

In some embodiments, h is 0. In some embodiments, h is 1. In some embodiments, h is 2. In some embodiments, h is 3. In some embodiments, h is 4.

In some embodiments, h is 0 or 1. In some embodiments, h is 1 or 2.

In some embodiments, I is 0. In some embodiments, I is 1. In some embodiments, I is 2. In some embodiments, I is 3. In some embodiments, I is 4.

In some embodiments, I is 0 or 1. In some embodiments, I is 1 or 2.

In some embodiments, j is 0. In some embodiments, j is 1. In some embodiments, j is 2. In some embodiments, j is 3. In some embodiments, j is 4.

In some embodiments, j is 0 or 1. In some embodiments, j is 1 or 2.

In some embodiments, k is 0. In some embodiments, k is 1. In some embodiments, k is 2. In some embodiments, k is 3. In some embodiments, k is 4.

In some embodiments, k is 0 or 1. In some embodiments, k is 1 or 2.

In certain embodiments, the present invention provides a compound of formula IV-c as any one of the following formula:

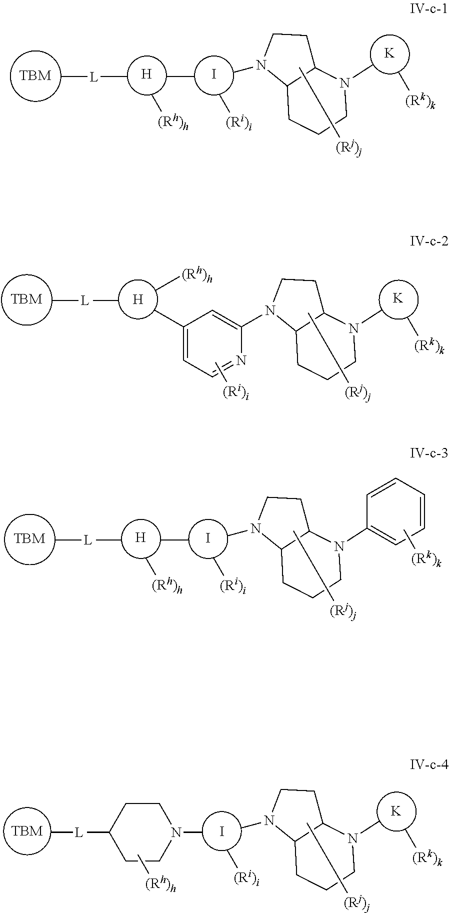

or a pharmaceutically acceptable salt thereof, wherein each of the variables is as defined above in formula TV-c and described in embodiments herein, both singly and in combination.

Exemplary compounds of the invention are set forth in Table 1, below.

Lengthy table referenced here
US20260125400A1-20260507-T00001
Please refer to the end of the specification for access instructions.

In some embodiments, the present invention provides a compound set forth in Table 1, above, or a pharmaceutically acceptable salt thereof.

In some embodiments, the invention also provides a compound described herein (such as a compound of formulae I-a to I-c), or pharmaceutical compositions described herein, for use in a method for degrading TYK2 as described herein and/or in a method for treating a TYK2-dependent disorder as described herein. In some embodiments, the invention also provides a compound described herein (such as a compound of formulae I-a to I-c), or pharmaceutical compositions described herein, for use in a method of degrading TYK2 as described herein. In some embodiments, the invention also provides a compound described herein (such as a compound of formulae I-a to I-c), or pharmaceutical compositions described herein, for use in a method of treating a TYK2-dependent disorder as described herein.

In some embodiments, the present invention provides a compound of formulae I-a to I-c as defined above, or a pharmaceutically acceptable salt thereof, or a pharmaceutical composition comprising a compound of formulae I-a to I-c as defined above, or a pharmaceutically acceptable salt thereof, and a pharmaceutically acceptable carrier, adjuvant, or vehicle for use as a medicament, such as for treating a TYK2-dependent disorder as described herein.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that it is effective to measurably degrade and/or inhibit TYK2, or a mutant thereof, in a biological sample or in a patient. In certain embodiments, the amount of compound in compositions of this invention is such that it is effective to measurably degrade and/or inhibit TYK2, or a mutant thereof, in a biological sample or in a patient. In certain embodiments, a composition of this invention is formulated for administration to a patient in need of such composition. In some embodiments, a composition of this invention is formulated for oral administration to a patient. In some embodiments, a composition of this invention is for use in the manufacture of a medicament for the treatment of a TYK2 mediated disorder, disease, or condition in a patient.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the degradation of TYK2 and/or inhibition of TYK2 kinase activity.

As used herein, the terms “TYK2-mediated” disorders, diseases, and/or conditions as used herein means any disease or other deleterious condition in which TYK2 or a mutant thereof, are known to play a role. Accordingly, another embodiment of the present invention relates to treating or lessening the severity of one or more diseases in which TYK2 or a mutant thereof, are known to playa role. Such TYK2-mediated disorders include but are not limited to autoimmune disorders and inflammatory disorders.

Compounds of the present disclosure can degrade TYK2 and therefore are useful for treating diseases wherein the underlying pathology is, wholly or partially, mediated by TYK2.

In some embodiments, the invention provides a method of modulating TYK2 in a subject or biological sample comprising administering to the subject, or contacting the biological sample with a provided compound. In some embodiments, modulating TYK2 comprises inhibiting or degrading TYK2.

In some embodiments, the invention provides a method of treating a TYK2-mediated disorder, disease, or condition in a subject in need thereof comprising administering to the subject a provided compound or its pharmaceutical composition or pharmaceutical dosage form.

The activity of a compound utilized in this invention as a degrader of TYK2, or a mutant thereof, may be assayed in vitro, in vivo or in a cell line. In vitro assays include assays that determine inhibition of either the phosphorylation activity and/or the subsequent functional consequences, or ATPase activity of activated TYK2, or a mutant thereof. Alternate in vitro assays quantitate the ability of the inhibitor to bind to TYK2. Inhibitor binding may be measured by radiolabeling the inhibitor prior to binding, isolating the inhibitor/TYK2 complex and determining the amount of radiolabel bound. Alternatively, inhibitor binding may be determined by running a competition experiment where new inhibitors are incubated with TYK2 bound to known radioligands. Representative in vitro and in vivo assays useful in assaying a TYK2 degrader and/or inhibitor include those described and disclosed herein.

TYK2 is a non-receptor tyrosine kinase member of the Janus kinase (JAKs) family of protein kinases. The mammalian JAK family consists of four members, TYK2, JAK1, JAK2, and JAK3. JAK proteins, including TYK2, are integral to cytokine signaling. TYK2 associates with the cytoplasmic domain of type I and type II cytokine receptors, as well as interferon types I and III receptors, and is activated by those receptors upon cytokine binding. Cytokines implicated in TYK2 activation include interferons (e.g. IFN-α, IFN-β, IFN-κ, IFN-δ, IFN—ε, IFN-τ, IFN-ω, and IFN-ξ (also known as limiting), and interleukins (e.g. IL-4, IL-6, IL-10, IL-11, IL-12, IL-13, IL-22, IL-23, IL-27, IL-31, oncostatin M, ciliary neurotrophic factor, cardiotrophin 1, cardiotrophin-like cytokine, and LIF). Velasquez et al., “A protein kinase in the interferon α/β signaling pathway,” Cell (1992) 70:313; Stahl et al., “Association and activation of Jak-Tyk kinases by CNTF-LIF-OSM-IL-60 receptor components,” Science (1994) 263:92; Finbloom et al., “IL-10 induces the tyrosine phosphorylation of TYK2 and JakI and the differential assembly of STAT1 and STAT3 complexes in human T cells and monocytes,” J. Immunol. (1995) 155:1079; Bacon et al., “Interleukin 12 (IL-12) induces tyrosine phosphorylation of Jak2 and Tyk2: differential use of Janus family kinases by IL-2 and IL-12,” J. Exp. Med. (1995) 181:399; Welham et al., “Interleukin-13 signal transduction in lymphohemopoietic cells: similarities and differences in signal transduction with interleukin-4 and insulin,” J. Biol. Chem. (1995) 270:12286; Parham et al., “A receptor for the heterodimeric cytokine IL-23 is composed of IL-12Rβ1 and a novel cytokine receptor subunit, IL-23R,” J. Immunol. (2002) 168:5699. The activated TYK2 then goes on to phosphorylate further signaling proteins such as members of the STAT family, including STAT1, STAT2, STAT4, and STAT6.

TYK2 activation by IL-23, has been linked to inflammatory bowel disease (IBD), Crohn's disease, and ulcerative colitis. Duerr et al., “A Genome-Wide Association Study Identifies IL23R as an Inflammatory Bowel Disease Gene,” Science (2006) 314:1461-1463. As the downstream effector of IL-23, TYK2 also plays a role in psoriasis, ankylosing spondylitis, and Behçet's disease. Cho et al., “Genomics and the multifactorial nature of human auto-immune disease,” N. Engl. J. Med (2011) 365:1612-1623; Cortes et al., “Identification of multiple risk variants for ankylosing spondylitis through high-density genotyping of immune-related loci,” Nat. Genet. (2013) 45(7):730-738; Remmers et al., “Genome-wide association study identifies variants in the MIIC class I, IL10, and IL23R-IL12RB2 regions associated with Behçet's disease,” Nat. Genet. (2010) 42:698-702. A genome-wide association study of 2,622 individuals with psoriasis identified associations between disease susceptibility and TYK2. Strange et al., “A genome-wide association study identifies new psoriasis susceptibility loci and an interaction between HLA-C and ERAP1,” Nat. Genet. (2010) 42:985-992. Knockout or tyrphostin inhibition of TYK2 significantly reduces both IL-23 and IL-22-induced dermatitis. Ishizaki et al., “Tyk2 is a therapeutic target for psoriasis-like skin inflammation,” Intl. Immunol. (2013), doi: 10.1093/intimm/dxt062.

TYK2 also plays a role in respiratory diseases such as asthma, chronic obstructive pulmonary disease (COPD), lung cancer, and cystic fibrosis. Goblet cell hyperplasia (GCH) and mucous hypersecretion is mediated by IL-13-induced activation of TYK2, which in turn activates STAT6. Zhang et al., “Docking protein Gab2 regulates mucin expression and goblet cell hyperplasia through TYK2/STAT6 pathway,” FASEB J. (2012) 26:1-11. Decreased TYK2 activity leads to protection of joints from collagen antibody-induced arthritis, a model of human rheumatoid arthritis. Mechanistically, decreased TYK2 activity reduced the production of Th1/Th17-related cytokines and matrix metalloproteases, and other key markers of inflammation. Ishizaki et al., “Tyk2 deficiency protects joints against destruction in anti-type II collagen antibody-induced arthritis in mice,” Intl. Immunol. (2011) 23(9):575-582.

TYK2 knockout mice showed complete resistance in experimental autoimmune encephalomyelitis (EAE, an animal model of multiple sclerosis (MS)), with no infiltration of CD4 T cells in the spinal cord, as compared to controls, suggesting that TYK2 is essential to pathogenic CD4-mediated disease development in MS. Oyamada et al., “Tyrosine Kinase 2 Plays Critical Roles in the Pathogenic CD4 T Cell Responses for the Development of Experimental Autoimmune Encephalomyelitis,” J. Immunol. (2009) 183:7539-7546. This corroborates earlier studies linking increased TYK2 expression with MS susceptibility. Ban et al., “Replication analysis identifies TYK2 as a multiple sclerosis susceptibility factor,” Eur J. Hum. Genet. (2009) 17:1309-1313. Loss of function mutation in TYK2, leads to decreased demyelination and increased remyelination of neurons, further suggesting a role for TYK2 inhibitors in the treatment of MS and other CNS demyelination disorders.

TYK2 is the sole signaling messenger common to both IL-12 and IL-23. TYK2 knockout reduced methylated BSA injection-induced footpad thickness, imiquimod-induced psoriasis-like skin inflammation, and dextran sulfate sodium or 2,4,6-trinitrobenzene sulfonic acid-induced colitis in mice.

Joint linkage and association studies of various type I IFN signaling genes with systemic lupus erythematosus (SLE, an autoimmune disorder), showed a strong, and significant correlation between loss of function mutations to TYK2 and decreased prevalence of SLE in families with affected members. Sigurdsson et al., “Polymorphisms in the Tyrosine Kinase 2 and Interferon Regulatory Factor 5 Genes Are Associated with Systemic Lupus Erythematosus,” Am. J. Hum. Genet. (2005) 76:528-537. Genome-wide association studies of individuals with SLE versus an unaffected cohort showed highly significant correlation between the TYK2 locus and SLE. Graham et al., “Association of NCF2, IKZF1, RF8, IFIHI, and TYK2 with Systemic Lupus Erythematosus,” PLoS Genetics (2011) 7(10):e1002341.

TYK2 has been shown to play an important role in maintaining tumor surveillance and TYK2 knockout mice showed compromised cytotoxic T cell response, and accelerated tumor development. However, these effects were linked to the efficient suppression of natural killer (NK) and cytotoxic T lymphocytes, suggesting that TYK2 inhibitors would be highly suitable for the treatment of autoimmune disorders or transplant rejection. Although other JAK family members such as JAK3 have similar roles in the immune system, TYK2 has been suggested as a superior target because of its involvement in fewer and more closely related signaling pathways, leading to fewer off-target effects. Simma et al. “Identification of an Indispensable Role for Tyrosine Kinase 2 in CTL-Mediated Tumor Surveillance,” Cancer Res. (2009) 69:203-211.

However, paradoxically to the decreased tumor surveillance observed by Simma et al., studies in T-cell acute lymphoblastic leukemia (T-ALL) indicate that T-ALL is highly dependent on IL-10 via TYK2 via STAT1-mediated signal transduction to maintain cancer cell survival through upregulation of anti-apoptotic protein BCL2. Knockdown of TYK2, but not other JAK family members, reduced cell growth. Specific activating mutations to TYK2 that promote cancer cell survival include those to the FERM domain (G36D, S47N, and R425H), the JH2 domain (V731I), and the kinase domain (E957D and R1027H). However, it was also identified that the kinase function of TYK2 is required for increased cancer cell survival, as TYK2 enzymes featuring kinase-dead mutations (M978Y or M978F) in addition to an activating mutation (E957D) resulted in failure to transform. Sanda et al. “TYK2-STAT1-BCL2 Pathway Dependence in T-Cell Acute Lymphoblastic Leukemia,” Cancer Disc. (2013) 3(5):564-577.

Thus, selective degradation and/or inhibition of TYK2 has been suggested as a suitable target for patients with IL-10 and/or BCL2-addicted tumors, such as 70% of adult T-cell leukemia cases. Fontan et al. “Discovering What Makes STAT Signaling TYK in T-ALL,” Cancer Disc. (2013) 3:494-496.

TYK2 mediated STAT3 signaling has also been shown to mediate neuronal cell death caused by amyloid-β (Aβ) peptide. Decreased TYK2 phosphorylation of STAT3 following Aβ administration lead to decreased neuronal cell death, and increased phosphorylation of STAT3 has been observed in postmortem brains of Alzheimer's patients. Wan et al. “Tyk/STAT3 Signaling Mediates β-Amyloid-Induced Neuronal Cell Death: Implications in Alzheimer's Disease,” J. Neurosci. (2010) 30(20):6873-6881.

Inhibition of JAK-STAT signaling pathways is also implicated in hair growth, and the reversal of the hair loss associated with alopecia areata. Xing et al., “Alopecia areata is driven by cytotoxic T lymphocytes and is reversed by JAK inhibition,” Nat. Med. (2014) 20: 1043-1049; Harel et al., “Pharmacologic inhibition of JAK-STAT signaling promotes hair growth,” Sci. Adv. (2015) 1(9):e1500973.

Accordingly, compounds that degrade TYK2 and/or inhibit the activity of TYK2 are beneficial, especially those with selectivity over JAK2. Such compounds should deliver a pharmacological response that favorably treats one or more of the conditions described herein without the side-effects associated with the inhibition of JAK2.

Even though TYK2 inhibitors are known in the art, there is a continuing need to provide novel inhibitors having more effective or advantageous pharmaceutically relevant properties. For example, compounds with increased activity, selectivity over other JAK kinases (especially JAK2), and ADMET (absorption, distribution, metabolism, excretion, and/or toxicity) properties. Thus, in some embodiments, the present invention provides degraders of TYK2 which show selectivity over JAK2.

Provided compounds are degraders and/or inhibitors of TYK2 and are therefore useful for treating one or more disorders associated with activity of TYK2 or mutants thereof. Thus, in certain embodiments, the present invention provides a method for treating a TYK2-mediated disorder comprising the step of administering to a patient in need thereof a compound of the present invention, or pharmaceutically acceptable composition thereof.

In some embodiments, the present invention provides a method for treating one or more disorders, wherein the disorders are selected from autoimmune disorders, inflammatory disorders, proliferative disorders, cardiovascular disease, endocrine disorders, neurological disorders, dysplasia related disorders, genetic disorders, and disorders associated with transplantation, said method comprising administering to a patient in need thereof, a pharmaceutical composition comprising an effective amount of a compound of the present invention, or a pharmaceutically acceptable salt thereof.

In some embodiments, the disorder is an autoimmune disorder. In some embodiments the disorder is selected from type 1 diabetes, systemic lupus erythematosus, multiple sclerosis, psoriasis, Behçet's disease, POEMS syndrome, Crohn's disease, ulcerative colitis, proteasome-associated autoinflammatory syndromes (PRAAS), ISG15 deficiency, and inflammatory bowel disease.

In some embodiments, the disorder is an inflammatory disorder. In some embodiments, the inflammatory disorder is rheumatoid arthritis, asthma, chronic obstructive pulmonary disease, psoriasis, hepatomegaly, Crohn's disease, ulcerative colitis, STING-associated vasculopathy with onset in infancy (SAVI), or inflammatory bowel disease.

In some embodiments, the disorder is a proliferative disorder. In some embodiments, the proliferative disorder is a hematological cancer. In some embodiments the proliferative disorder is a leukemia. In some embodiments, the leukemia is a T-cell leukemia. In some embodiments the T-cell leukemia is T-cell acute lymphoblastic leukemia (T-ALL). In some embodiments the proliferative disorder is polycythemia vera, myelofibrosis, essential or thrombocytosis.

In some embodiments, the disorder is a cardiovascular disease. In some embodiments, the cardiovascular disease is atherosclerosis. In some embodiments, the cardiovascular disease is premature atherosclerosis.

In some embodiments, the disorder is an endocrine disorder. In some embodiments, the endocrine disorder is polycystic ovary syndrome, Crouzon's syndrome, or type 1 diabetes.

In some embodiments, the disorder is a neurological disorder. In some embodiments, the neurological disorder is Alzheimer's disease or Aicardi-Goiuteieres syndrome (AGS).

In some embodiments the proliferative disorder is associated with one or more activating mutations in TYK2. In some embodiments, the activating mutation in TYK2 is a mutation to the FERM domain, the JH2 domain, or the kinase domain. In some embodiments the activating mutation in TYK2 is selected from G36D, S47N, R425H, V731I, E957D, and R1027H.

In some embodiments, the disorder is a dysplasia related disorder. In some embodiments, the dysplasia related disorder is spondyloenchondrodysplasia (SPENCD), such as spondyloenchondrodysplasia with immune dysregulation (SPENCDI).

In some embodiments, the disorder is a dysplasia related disorder. In some embodiments, the dysplasia related disorder is a genetic disorder such as Singleton-Merten syndrome (SMS).

In some embodiments, the disorder is associated with transplantation. In some embodiments the disorder associated with transplantation is transplant rejection, or graft versus host disease.

In some embodiments the disorder is associated with type I interferon, IL-10, IL-12, or IL-23 signaling. In some embodiments the disorder is associated with type I interferon signaling. In some embodiments the disorder is associated with IL-10 signaling. In some embodiments the disorder is associated with IL-12 signaling. In some embodiments the disorder is associated with IL-23 signaling, such as psoriasis, psoriatic arthritis, ankylosing spondylitis, Crohn's disease, and ulcerative colitis.

Compounds of the invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g., hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g., ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis (e.g., anti-neutrophil cytoplasmic antibody (ANCA)-associated vasculitis (AAV) and polyarteritis nodosa (PAN)), diverticulitis, interstitial cystitis, glomerulonephritis (with and without nephrotic syndrome, e.g., including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, or vulvitis.

In some embodiments the inflammatory disease which can be treated according to the methods of this invention is selected from acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), and osteoarthritis.

In some embodiments the inflammatory disease which can be treated according to the methods of this invention is a Th1 or Th17 mediated disease. In some embodiments the Th17 mediated disease is selected from systemic lupus erythematosus, Multiple sclerosis, and inflammatory bowel disease (including Crohn's disease or ulcerative colitis).

In some embodiments the inflammatory disease which can be treated according to the methods of this invention is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

Furthermore, the invention provides the use of a compound according to the definitions herein, or a pharmaceutically acceptable salt, or a hydrate or solvate thereof for the preparation of a medicament for the treatment of an autoimmune disorder, an inflammatory disorder, or a proliferative disorder, or a disorder commonly occurring in connection with transplantation.

In some embodiments, the present invention provides a method of treating pain in a patient in need thereof, the method comprising administering to the patient a compound of the present invention or a therapeutically acceptable salt thereof.

In some embodiments, the present invention provides a method of treating inflammation in a patient in need thereof, the method comprising administering to the patient a compound of the present invention or a therapeutically acceptable salt thereof.

In some embodiments, the present invention provides a method of treating tissue damage in a patient in need thereof, the method comprising administering to the patient a compound of the present invention or a therapeutically acceptable salt thereof.

In some embodiments, the present invention provides a method of treating arthritis in a patient in need thereof, the method comprising administering to the patient a compound of the present invention or a therapeutically acceptable salt thereof.

It is believed that a provided compound or a pharmaceutically acceptable salt thereof may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

In some embodiments, the compounds of the invention are used to treat the TYK2-mediated disorder.

In some embodiments, the compounds of the invention are used to treat the TYK2-mediated disorder, wherein the disorder is an inflammatory disorder, a proliferative disorder, an endocrine disorder, a neurological disorder, or a disorder associated with transplantation.

In some embodiments, the compounds of the invention are used to treat the TYK2-mediated disorder, wherein the disorder is an autoimmune disorder selected from type 1 diabetes, ankylosing spondylitis, cutaneous lupus erythematosus, systemic lupus erythematosus, lupus nephritis, multiple sclerosis, systemic sclerosis, psoriasis, Crohn's disease, ulcerative colitis, proteasome-associated autoinflammatory syndromes (PRAAS), ISG15 deficiency, and inflammatory bowel disease.

In some embodiments, the compounds of the invention are used to treat the TYK2-mediated disorder, wherein the disorder is an inflammatory disorder selected from rheumatoid arthritis, asthma, chronic obstructive pulmonary disease, psoriasis, Crohn's disease, ulcerative colitis, STING-associated vasculopathy with onset in infancy (SAVI), and inflammatory bowel disease.

In some embodiments, the compounds of the invention are used to treat the TYK2-mediated disorder, wherein the disorder is a proliferative disorder selected from a hematological cancer, polycythemia vera, myelofibrosis, essential thrombocythemia, and thrombocytosis.

In some embodiments, the compounds of the invention are used to treat the TYK2-mediated disorder, wherein the disorder is an endocrine disorder selected from polycystic ovary syndrome, Crouzon's syndrome, and type 1 diabetes.

In some embodiments, the compounds of the invention are used to treat the TYK2-mediated disorder, wherein the disorder is a neurological disorder selected from Alzheimer's disease, Aicardi-Goiuteieres syndrome (AGS), Parkinson's disease, amyotrophic lateral sclerosis, Huntington's disease, cerebral ischemia, and neurodegenerative disease caused by traumatic injury, glutamate neurotoxicity and hypoxia.

In some embodiments, the compounds of the invention are used to treat the TYK2-mediated disorder, wherein the disorder is associated with transplantation selected from transplant rejection and graft versus host disease.

In some embodiments, the compounds of the invention are used to a subject that is human.

Co-Administration with One or More Other Therapeutic Agent(s)

Depending upon the particular condition, or disease, to be treated, additional therapeutic agents that are normally administered to treat that condition, can also be present in the compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

A compound of the current invention can also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides, or in addition, be administered especially for tumor therapy in combination with chemotherapy, radiotherapy, immunotherapy, phototherapy, surgical intervention, or a combination of these. Long-term therapy is equally possible, as is adjuvant therapy in the context of other treatment strategies, as described above. Other possible treatments are therapy to maintain the patient's status after tumor regression, or even chemopreventive therapy, for example in patients at risk.

One or more other therapeutic agent(s) can be administered separately from a compound or composition of the invention, as part of a multiple dosage regimen. Alternatively, one or more other therapeutic agent(s) may be part of a single dosage form, mixed together with a compound of this invention in a single composition. If administered as a multiple dosage regime, one or more other therapeutic agent(s) and a compound or composition of the invention can be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent(s) and a compound or composition of the invention are administered as a multiple dosage regimen within greater than 24 hours apart.

As used herein, the term “combination,” “combined,” and related terms refers to the simultaneous or sequential administration of therapeutic agents in accordance with this invention. For example, a compound of the present invention can be administered with one or more other therapeutic agent(s) simultaneously or sequentially in separate unit dosage forms or together in a single unit dosage form. Accordingly, the present invention provides a single unit dosage form comprising a compound of the current invention, one or more other therapeutic agent(s), and a pharmaceutically acceptable carrier, adjuvant, or vehicle.

The amount of a compound of the invention and one or more other therapeutic agent(s) (in those compositions which comprise an additional therapeutic agent as described above) that can be combined with the carrier materials to produce a single dosage form varies depending upon the host treated and the particular mode of administration. Preferably, a composition of the invention should be formulated so that a dosage of between 0.01-100 mg/kg body weight/day of a compound of the invention can be administered.

In those compositions which comprise one or more other therapeutic agent(s), the one or more other therapeutic agent(s) and a compound of the invention can act synergistically. Therefore, the amount of the one or more other therapeutic agent(s) in such compositions may be less than that required in a monotherapy utilizing only that therapeutic agent. In such compositions a dosage of between 0.01-1,000 g/kg body weight/day of the one or more other therapeutic agent(s) can be administered.

The amount of one or more other therapeutic agent(s) present in the compositions of this invention may be no more than the amount that would normally be administered in a composition comprising that therapeutic agent as the only active agent. Preferably the amount of one or more other therapeutic agent(s) in the presently disclosed compositions ranges from about 50% to 100% of the amount normally present in a composition comprising that agent as the only therapeutically active agent. In some embodiments, one or more other therapeutic agent(s) is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, can also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Other Therapeutic Agents

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex© and Rebif©), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

In another embodiment, the present invention provides a method of treating an inflammatory disease, disorder or condition by administering to a patient in need thereof a provided compound and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-Bid®, Uniphyl®, Theo-24R) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, colchicine (Colcrvs®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-I” agents such as anakinra (Kineret®) and rilonacept (Arcalyst®), antibodies such as rituximab (Rituxan®). “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a provided compound and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-Bid®, Uniphyl®, Theo-24®) and aminophylline, and IgE antibodies such as omalizumab (Xolair®).

In some embodiments, the present invention provides a method of treating COPD comprising administering to a patient in need thereof a provided compound and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-Bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In some embodiments, the present invention provides a method of treating HIV comprising administering to a patient in need thereof a provided compound and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In another embodiment, the present invention provides a method of treating a hematological malignancy comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, an alternative TYK2 inhibitor, a PI3K inhibitor, a SYK inhibitor, and combinations thereof.

In another embodiment, the present invention provides a method of treating a solid tumor comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, an alternative TYK2 inhibitor, a PI3K inhibitor, a SYK inhibitor, and combinations thereof.

In another embodiment, the present invention provides a method of treating a hematological malignancy comprising administering to a patient in need thereof a provided compound and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a provided compound and one or more additional therapeutic agents selected from bortezomib (Velcade®), and dexamethasone (Decadron®), a hedgehog signaling inhibitor, a BTK inhibitor, a JAK/pan-JAK inhibitor, an alternative TYK2 inhibitor, a PI3K inhibitor, a SYK inhibitor in combination with lenalidomide (Revlimid®).

In another embodiment, the present invention provides a method of treating Waldenström's macroglobulinemia comprising administering to a patient in need thereof a provided compound and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, an alternative TYK2 inhibitor, a PI3K inhibitor, and a SYK inhibitor.

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a Poly ADP ribose polymerase (PARP) inhibitor. In some embodiments, a PARP inhibitor is selected from olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis); abemaciclib (Ly2835219, Eli Lilly); and trilaciclib (G1T28, G1 Therapeutics).

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, DarzalexR, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®. Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFI3). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int′l J. Biological Sciences 8:964-978. One therapeutic compound currently in clinical trials for treatment of solid tumors is M7824 (Merck KgaA—formerly MSB0011459X), which is a bispecific, anti-PD-L1/TGFI3 trap compound (NCT02699515); and (NCT02517398). M7824 is comprised of a fully human IgG1 antibody against PD-L1 fused to the extracellular domain of human TGF-beta receptor II, which functions as a TGFI3 “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx;

MEK inhibitors such as ARRY142886 from Array BioPharma, AZd6244 from AstraZeneca, PD181461 from Pfizer and leucovorin.

In some embodiments, the present invention provides a method of treating Alzheimer's disease comprising administering to a patient in need thereof a provided compound and one or more additional therapeutic agents selected from donepezil (Aricept®), rivastigmine (Excelon®), galantamine (Razadyne®), tacrine (Cognex®), and memantine (Namenda®).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (TaxotereR, Sanofi-Aventis; Docefrez®, Sun Pharmaceutical), albumin-bound paclitaxel (AbraxaneR; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaceuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547.632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs, host disease comprising administering to a patient in need thereof a provided compound and one or more additional therapeutic agents selected from a steroid, cyclosporin, FK506, rapamycin, a hedgehog signaling inhibitor, a BTK inhibitor, a JAK/pan-JAK inhibitor, an alternative TYK2 inhibitor, a PI3K inhibitor, and a SYK inhibitor.

In another embodiment, the present invention provides a method of treating or lessening the severity of a disease comprising administering to a patient in need thereof a provided compound and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis (including juvenile arthritis with lung disease), diabetes, myasthenia gravis. Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takavasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, seleraderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound and a PI3K inhibitor, wherein the disease is selected from a cancer, a neurodegenative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g, hemolytic anemia, aplastic anemia, pure red cell anemia and idiopathic thrombocytopenia), systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g, including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g, in the form as it is marketed, e.g. under the trademark Camptosar. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed, under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™ Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ, of Michigan), and venetoclax. In some embodiments the Bcl-2 inhibitor is a small molecule therapeutic. In some embodiments the Bcl-2 inhibitor is a peptidomimetic.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g., BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR1 ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, C₁₋₁₀₃₃, EKB-569, GW-2016, ELI, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Ky, PI3K6, PI3K13, PI3K-C2α, PI3K-C213, PI3K-C2γ, Vps34, p110-α, p110-13, p110-γ, p110-6, p85-α, p85-13, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g, thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™ Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to. Bortezomib (Velcade™)); carfilzomib (Kyprolis®, Amgen); and ixazomib (Ninlaro®, Takeda), and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-p3-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idambicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]- amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4th Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly, the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering- Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH—55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2). B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTJ3R, LIGHT, DcR3, HVEM, VEGI/TL1A, TRAMP/DR3, EDAR, EDA1, XEDAR, EDA2, TNFR1, Lymphotoxin α/TNFJ3, TNFR2, TNFα, LTJ3R, Lymphotoxin α1J32, FAS, FASL, RELT, DR6, TROY, NGFR.

In some embodiments, an immuno-oncology agent is a cytokine that inhibits T cell activation (e.g., IL-6, IL-10, TGF-J3, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2. LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27. CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13/169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fe portion of IgG1, called AMP-224.

In some embodiments, an immuno-oncology agent is a PD-L1 antagonist. In some embodiments, a PD-L1 antagonist is an antagonistic PD-Li antibody. In some embodiments, a PD-Li antibody is MPDL3280A (RG7446; WO2010/077634), durvalumab (MED14736), BMS-936559 (WO2007/005874), and MSB001071SC (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T, et al. (2013) Nat. Immunol. 14, 1212-1218; Zou et al. (2016) Sci. Transl. Med. 8. The anti-PD-1 antibody nivolumab (OpdivoR, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCTO1166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+(Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 1 of Jerry L. Adams ET. AL. In some embodiments, an immuno-oncology agent is a small molecule agent selected from those listed in Table 1 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the contents of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1. B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8+ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4. B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PDL1 antibody), BY55 monoclonal antibody, AMP224 (anti-PDL1 antibody), BMS- 936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224. MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-Li antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MED10562 (Medimmune/AstraZeneca), an agonistic anti-OX40 antibody, in advanced solid tumors (NCT02318394 and NCT02705482); MED16469, an agonistic anti-OX40 antibody (Medimmune/AstraZeneca), in patients with colorectal cancer (NCT02559024), breast cancer (NCTO1862900), head and neck cancer (NCT02274155) and metastatic prostate cancer (NCTO1303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol- Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgGI Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MED19447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

In some embodiments, TYK2 inhibition/degradation can significantly enhance CDN-induced STING signaling and antitumor immunity (Pei et al., Can. Lett. 2019, 450:110).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

### EXEMPLIFICATION

### Abbreviations

Ac: acetyl
AcOH: acetic acid
ACN: acetonitrile
Ad: adamantyl
AIBN: 2,2′-azo bisisobutyronitrile
Anhyd: anhydrous
Aq: aqueous
B₂Pin₂: bis (pinacolato)diboron-
4,4,4′,4′,5,5,5′,5′-octamethyl-2,2′-bi(1,3,2-dioxaborolane)
BINAP: 2,2′-bis(diphenylphosphino)-1,1′-binaphthyl
BH₃: Borane
Bn: benzyl
Boc: tert-butoxycarbonyl
Boc₂O: di-tert-butyl dicarbonate
BPO: benzoyl peroxide
ⁿBuOH: n-butanol
CDI: carbonyldiimidazole
CMPB: 2-(tributyl-lambda5-phosphanylidene)acetonitrile
COD: cyclooctadiene
d: days
DABCO: 1,4-diazobicyclo[2.2.2]octane
DAST: diethylaminosulfur trifluoride
dba: dibenzylideneacetone
DBU: 1,8-diazobicyclo[5.4.0]undec-7-ene
DCE: 1,2-dichloroethane
DCM: dichloromethane
DEA: diethylamine
DHP: dihydropyran
DIBAL-H: diisobutylaluminum hydride
DIPA: diisopropylamine
DIPEA or DIEA: N,N-diisopropylethylamine
DMA: N,N-dimethylacetamide
DME: 1,2-dimethoxyethane
DMAP: 4-dimethylaminopyridine
DMF: N,N-dimethylformamide
DMP: Dess-Martin periodinane
DMSO-dimethyl sulfoxide
DPPA: diphenylphosphoryl azide
dppf: 1,1′-bis(diphenylphosphino)ferrocene
EDC or EDCI: 1-(3-dimethylaminopropyl)-3-
ethylcarbodiimide hydrochloride
ee: enantiomeric excess
ESI: electrospray ionization
EA: ethyl acetate
EtOAc: ethyl acetate
EtOH: ethanol
FA: formic acid
h or hrs: hours
HATU: N,N,N′,N′-tetramethyl-O-(7-azabenzotriazol-1-
yl)uranium hexafluorophosphate
HCl: hydrochloric acid
HPLC: high performance liquid chromatography
HOAc: acetic acid
HOBT or HOBt: hydroxybenzotriazole
IBX: 2-iodoxybenzoic acid
IPA: isopropyl alcohol
KHMDS: potassium hexamethyldisilazide
K₂CO₃: potassium carbonate
LAH: lithium aluminum hydride
LDA: lithium diisopropylamide
m-CPBA: meta-chloroperbenzoic acid
M: molar
MeCN: acetonitrile
MeOH: methanol
Me₂S: dimethyl sulfide
MeONa: sodium methylate
MeI: iodomethane
min: minutes
mL: milliliters
mM: millimolar
mmol: millimoles
MPa: mega pascal
MOMCl: methyl chloromethyl ether
MsCl: methanesulfonyl chloride
MTBE: methyl tert-butyl ether
nBuLi: n-butyllithium
NaNO₂: sodium nitrite
NaOH: sodium hydroxide
Na₂SO₄: sodium sulfate
NBS: N-bromosuccinimide
NCS: N-chlorosuccinimide
NFSI: N-Fluorobenzenesulfonimide
NMO: N-methylmorpholine N-oxide
NMP: N-methylpyrrolidine
NMR: Nuclear Magnetic Resonance
° C.: degrees Celsius
Pd/C: Palladium on Carbon
Pd(OAc)₂: Palladium Acetate
PBS: phosphate buffered saline
PE: petroleum ether
POCl₃: phosphorus oxychloride
Pd-PEPPSI-IPentCl: {1,3-bis[2,6-bis(pentan-3-
yl)phenyl]-4,5-dichloro-2,3-dihydro-1H-imidazol-2-
yl}dichloro(2-methyl-1lambda4-pyridin-1-yl)palladium
PPh₃: triphenylphosphine
PyBOP: (Benzotriazol-1-yloxy)tripyrrolidinophosphonium
hexafluorophosphate
Rel: relative
R.T. or rt: room temperature
RuPhos: 2-Dicyclohexylphosphino-2′,6′-diisopropoxybiphenyl
sat: saturated
SEMCl: chloromethyl-2-trimethylsilylethyl ether
SFC: supercritical fluid chromatography
SOCl₂: sulfur dichloride
tBuOK: potassium tert-butoxide
TBAB: tetrabutylammonium bromide
TBAI: tetrabutylammonium iodide
TEA: triethylamine
Tf: trifluoromethanesulfonate
TfAA, TFMSA or Tf₂O: trifluoromethanesulfonic anhydride
TFA: trifluoracetic acid
TIPS: triisopropylsilyl
THF: tetrahydrofuran
THP: tetrahydropyran
TLC: thin layer chromatography
TMEDA: tetramethylethylenediamine
TosMIC: toluenesulfonylmethyl isocyanide
pTSA: para-toluenesulfonic acid
wt: weight
Xantphos: 4,5-bis(diphenylphosphino)-9,9-dimethylxanthene

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (═20-133 mbar). The structure of final products, intermediates and starting materials is confirmed by standard analytical methods, e.g., microanalysis and spectroscopic characteristics, e.g., MS. IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

TABLE 2
Analytical instruments

| | |
|---|---|
| LCMS | Shimadzu UFLC MS: LCMS-2020 |
| | Agilent Technologies 1200 series MS: Agilent |
| | Technologies 6110 |
| | Agilent Technologies 1200 series MS: LC/MSD VL |
| NMR | BRUKER AVANCE III/400; Frequency (MHz) |
| | 400.13; Nucleus: 1H; Number of Transients: 8 |
| Prep-HPLC | Gilson GX-281 systems: instruments GX-A, |
| | GX-B, GX-C, GX-D, GX-E, GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

For acidic LCMS data: LCMS was recorded on an Agilent 1200 Series LC/MSD or Shimadzu LCMS2020 equipped with electro-spray ionization and quadruple MS detector [ES+ve to give MH⁺] and equipped with Chromolith Flash RP-18e 25*2.0 mm, eluting with 0.0375 vol % TFA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A) 2 mM Ammonium Acetate in 0.1% Formic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH⁺] and equipped with Xbridge C18, 2.1×50 mm columns packed with 5 mm C18-coated silica or Kinetex EVO C18 2.1X³⁰ mm columns packed with 5 mm C18-coated silica, eluting with 0.05 vol % NH₃—H₂O in water (solvent A) and acetonitrile (solvent B).

HPLC Analytical Method: HPLC was carried out on X Bridge C18 150*4.6 mm, 5 micron. Column flow was 1.0 ml/min and mobile phase were used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19)mm, 5. Column flow was 16.0 ml/min. Mobile phase were used (A) 0.1% Formic Acid in Water and (B) Acetonitrile Basic method used (A) 5 mM ammonium bicarbonate and 0.1% NH3 in Water and (B) Acetonitrile or (A) 0.1% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Intermediates

The following intermediates were synthesized as described in WO 2023/076161, the content of which is herein incorporated by reference.

### 3-(5-bromo-3-methyl-2-oxo-benzimidazol-1-yl)piperidine-2,6-dione (Intermediate C)

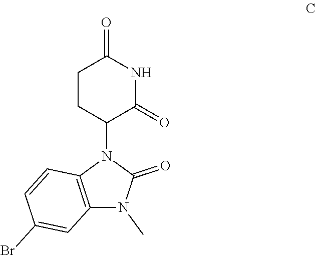 [SMILES: Cn1c(=O)n(C2CCC(=O)NC2=O)c2ccc(Br)cc21]

### 6-Chloro-8-{[(4-methoxyphenyl)methyl](methyl)amino}imidazo[1,2-b]pyridazine-3-carboxylic acid (Intermediate F)

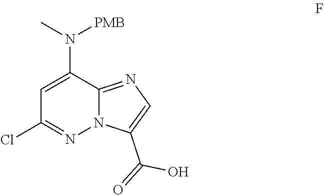

### 6-chloro-N-[(1R,2S)-2-fluorocyclopropyl]-8-{[(4-methoxyphenyl)methyl](methyl)amino}imidazo[1,2-b]pyridazine-3-carboxamide (Intermediate G)

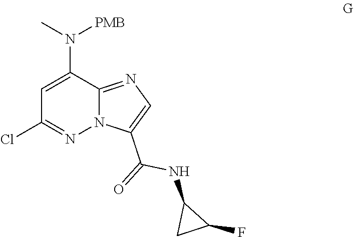

### 3-(4-Methoxybenzyl)dihydropyrimidine-2,4(1H,3H)-dione (Intermediate ID)

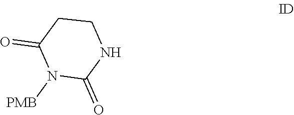

### 1-(2-Chloro-5-{2,7-diazaspiro[3.5]nonane-7-carbonyl}phenyl)-1,3-diazinane-2,4-dione (Intermediate IO)

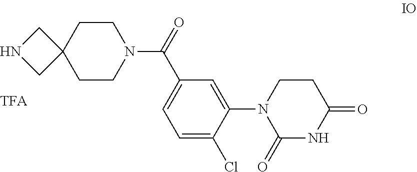 [SMILES: O=C1CCN(c2cc(C(=O)N3CCC4(CC3)CNC4)ccc2Cl)C(=O)N1]

### 3-[3-methyl-2-oxo-5-(piperazin-1-yl)-1,3-benzodiazol-1-yl]piperidine-2,6-dione (Intermediate T)

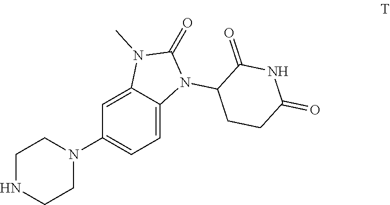 [SMILES: Cn1c(=O)n(C2CCC(=O)NC2=O)c2ccc(N3CCNCC3)cc21]

### 3-(4-Bromo-3-methyl-2-oxo-2,3-dihydro-1H-benzo[d]imidazol-1-yl)piperidine-2,6-dione (Intermediate U)

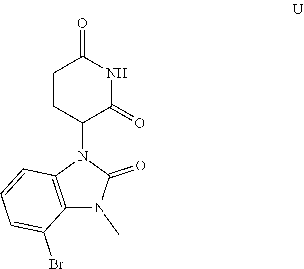 [SMILES: Cn1c(=O)n(C2CCC(=O)NC2=O)c2cccc(Br)c21]

### N-[(1R,2S)-2-fluorocyclopropyl]-6-(4-formyl-2,3-dihydroindol-1-yl)-8-(methylamino)imidazo[1,2-b]pyridazine-3-carboxamide (Intermediate KO)

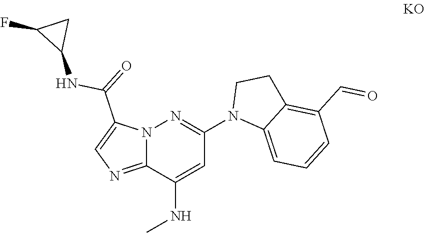 [SMILES: CNc1cc(N2CCc3c(C=O)cccc32)nn2c(C(=O)N[C@@H]3C[C@@H]3F)cnc12]

### 1-(1-(2,6-dioxopiperidin-3-yl)-3-methyl-2-oxo-2,3-dihydro-1H-benzo[d]imidazol-4-yl)piperidine-4-carbaldehyde (Intermediate KP)

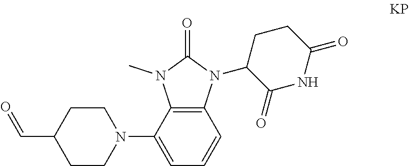 [SMILES: Cn1c(=O)n(C2CCC(=O)NC2=O)c2cccc(N3CCC(C=O)CC3)c21]

### 3-[3-methyl-2-oxo-4-(piperazin-1-yl)-1,3-benzodiazol-1-yl]piperidine-2,6-dione (Intermediate BW)

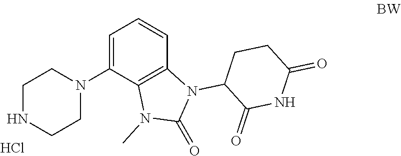 [SMILES: Cl ; Cn1c(=O)n(C2CCC(=O)NC2=O)c2cccc(N3CCNCC3)c21]

### N-((1R,2S)-2-fluorocyclopropyl)-6-(4-(5-formylpyridin-2-yl)indolin-1-yl)-8-(methylamino)imidazo[1,2-b]pyridazine-3-carboxamide (Intermediate IN)

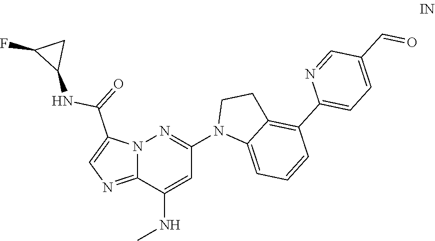 [SMILES: CNc1cc(N2CCc3c(-c4ccc(C=O)cn4)cccc32)nn2c(C(=O)N[C@@H]3C[C@@H]3F)cnc12]

### Ethyl 2-(5-amino-2-oxo-1-pyridyl)acetate (Intermediate A1)

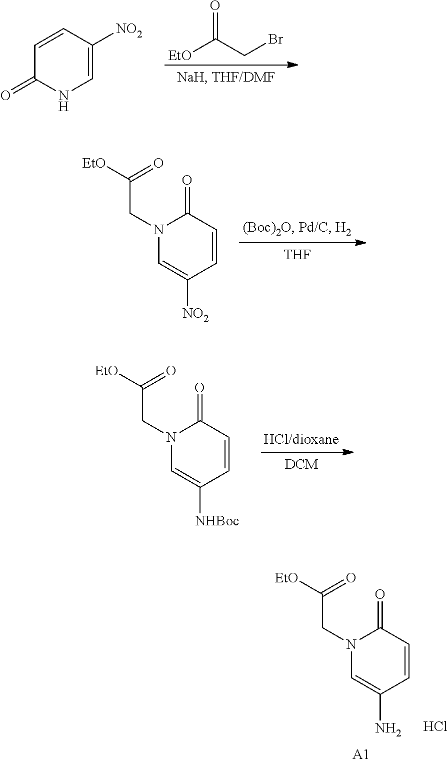

Step 1—Ethyl 2-(5-nitro-2-oxo-1-pyridyl)acetate. To a solution of 5-nitro-1H-pyridin-2-one (5.00 g, 35.7 mmol, CAS #5418-51-9) in THF (70 mL) and DMF (10 mL) was added NaH (1.71 g, 42.8 mmol, 60% dispersion in mineral oil), then the mixture was stirred at 0° C., for 1 hr. Next, ethyl 2-bromoacetate (6.56 g, 39, mmol, CAS #105-36-2) was added, and the mixture was stirred at 25° C., for 1 hr. On completion, saturated NH4Cl was added to the mixture to adjust the pH to 6. Then the mixture was diluted with water (20 mL), filtered to give the filter cake and dried in vacuo. The crude product was purified by column chromatography (SiO2, PE: EA=1:0 to 0:1) to give the title compound (6.20 g, 77% yield) as white solid.

1H NMR (400 MHz, DMSO-d6) δ 9.25 (d, J=3.2 Hz, 1H), 8.20-8.10 (m, 1H), 6.55 (d, J=10.0 Hz, 1H), 4.86 (s, 2H), 4.17 (q, J=7.2 Hz, 2H), 1.21 (t. J=7.2 Hz, 3H); LC-MS (ESI+) m/z 226.9 (M+H)+.

Step 2—Ethyl 2-[5-(tert-butoxycarbonylamino)-2-oxo-1-pyridyl]acetate. To a solution of ethyl 2-(5-nitro-2-oxo-1-pyridyl)acetate (1 g, 4.42 mmol) in THF (10 mL) was added (Boc)₂₀ (2.89 g, 13.2 mmol) and Pd/C (1 g, 4.42 mmol, 10 wt %) under N2. The suspension was degassed under vacuum and purged with H2 three times. The mixture was stirred under H2 (15 psi) at 25° C., for 2 hours. On completion, the reaction mixture was filtered and concentrated in vacuo to give the residue. The residue was purified by column chromatography (SiO2, PE/EA=20/1 to 1/1) to give the title compound (985 mg, 75% yield) as red solid. 1H NMR (400 MHz, DMSO-d6) δ 9.05 (s, 1H), 7.87 (s, 1H), 7.39 (m, 1H), 6.39 (d, J=9.6 Hz, 1H), 4.67 (s, 2H), 4.13 (q, J=7.2 Hz, 2H), 1.45 (s, 9H), 1.20 (t, J=7.2 Hz, 3H); LC-MS (ESI+) m/z 297.0 (M+H)+.

Step 3—Ethyl 2-(5-amino-2-oxo-1-pyridyl) acetate. To a solution of ethyl 2-[5-(tert-butoxycarbonylamino)-2-oxo-1-pyridyl]acetate (500 mg, 1.69 mmol) in DCM (5 mL) was added HCL/dioxane (4 M, 662 uL), then the mixture was stirred at 25° C., for 10 hrs. On completion, the reaction mixture was concentrated in vacuo to give the title compound (330 mg, 84% yield, HCl) as yellow solid. LC-MS (ESI+) m/z 197.1 (M+H)+.

### 2-(2-(2-(2-bromoethoxy)ethoxy)ethyl)isoindoline-1,3-dione (Intermediate B)

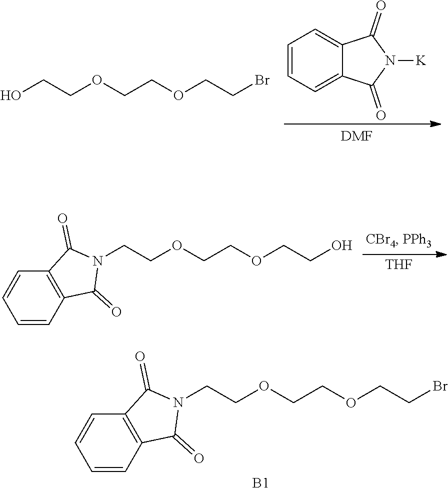

Step 1—2-(2-(2-(2-Hydroxyethoxy)ethoxy)ethyl)isoindoline-1,3-dione. To a stirred solution of potassium 1,3-dioxoisoindolin-2-ide (3.19 g, 21.7 mmol) in DMF (30 mL) was added 2-(2-(2-bromoethoxy)ethoxy)ethan-1-ol (4.80 g, 22.55 mmol) at rt. The resulting mixture was stirred for 2 h at 70° C. under nitrogen atmosphere. On completion, the mixture was cooled to rt and filtered, then the filter cake was washed with MeCN (3×30 mL). The filtrate was concentrated under reduced pressure. The residue was purified by reverse phase flash chromatography (Column: WelFlash™ C18-I, 20-40 m, 330 g; Eluent A: Water (plus 10 mmol/L FA); Eluent B: MeCN; Gradient: 20%-50% B in 35 min; Flow rate: 80 mL/min; Detector: 220/254 nm; desired fractions were collected at 30% B) and concentrated under reduced pressure to afford the title compound (5.30 g, 88% yield) as a yellow oil. LC/MS (ESI, m/z): [(M+1)]+=280.0.

Step 2—2-(2-(2-(2-bromoethoxy)ethoxy)ethyl)isoindoline-1,3-dione. To a stirred solution of 2-(2-(2-(2-hydroxyethoxy)ethoxy)ethyl)isoindoline-1,3-dione (5.20 g, 18.6 mmol) in THF (50 mL) was added PPh3 (9.77 g, 37.2 mmol) at rt. Then CBr4 (12.35 g, 37.24 mmol) was added at 0° C. The resulting mixture was then stirred for 2 h at rt under nitrogen atmosphere. On completion, the mixture was concentrated under reduced pressure. The residue was purified by silica gel column chromatography, eluted with petroleum ether/EtOAc (1:1) to afford the title compound (5.00 g, 78% yield) as a brown oil. LC/MS (ESI, m/z): [(M+1)]⁺=342.2, 244.2 (—BOC).

### Ethyl 2-(5-((2-(2-(2-(1,3-dioxoisoindolin-2-yl)ethoxy)ethoxy)ethyl)amino)-2-oxopyridin-1(2H)-yl)acetate hydrochloride (Intermediate C1)

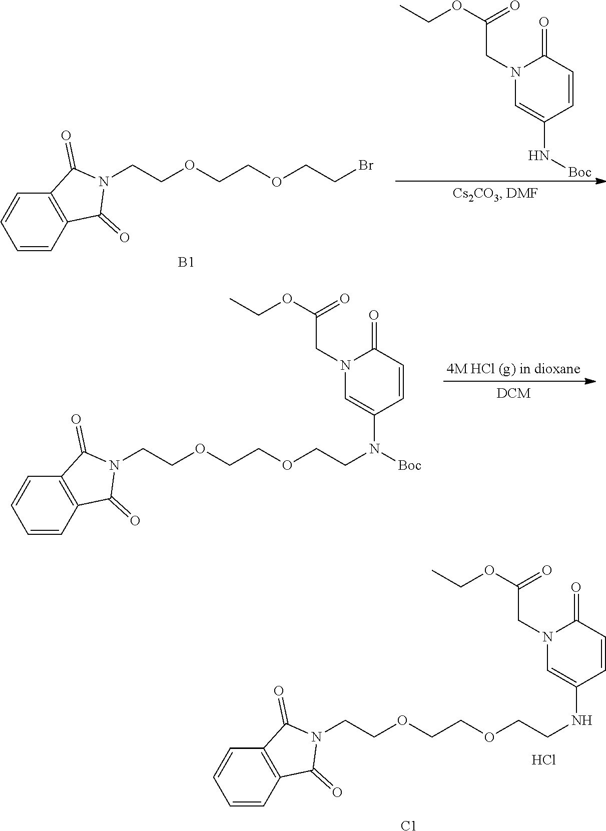

Step 1—ethyl 2-(5-((tert-butoxycarbonyl)(2-(2-(2-(1,3-dioxoisoindolin-2-yl)ethoxy)ethoxy)ethyl)amino)-2-oxopyridin-1(2H)-yl)acetate. To a stirred solution of 2-(2-(2-(2-bromoethoxy)ethoxy)ethyl)isoindoline-1,3-dione (5.00 g, 14.6 mmol, Intermediate B1) and ethyl 2-(5-((tert-butoxycarbonyl)amino)-2-oxopyridin-1(2H)-yl)acetate (4.76 g, 16.0 mmol, synthesized via Steps 1-2 of Intermediate A1) in DMF (20 mL) was added Cs2CO3 (14.28 g, 43.84 mmol) at rt under nitrogen atmosphere. The resulting mixture was stirred for an additional 1 h at 50° C. On completion, the mixture was cooled to rt and filtered, then the filter cake was washed with MeCN (3×30 mL). The filtrate was concentrated under reduced pressure. The residue was purified by reverse phase flash chromatography (Column: WelFlash™ C18-I, 20-40 μm, 330 g; Eluent A: Water (plus 10 mmol/L NH4HCO3); Eluent B: MeCN; Gradient: 25%-55% B in 25 min; Flow rate: 80 mL/min; Detector: 220/254 nm; desired fractions were collected at 43% B) and concentrated under reduced pressure to afford the title compound (2.80 g, 34% yield) as a brown oil. LC/MS (ESI, m/z): [(M+1)]⁺=558.2.

Step 2—Ethyl 2-(5-((2-(2-(2-(1,3-dioxoisoindolin-2-yl)ethoxy)ethoxy)ethyl)amino)-2-oxopyridin-1(2H)-yl)acetate hydrochloride. To a stirred solution of 2-(5-((tert-butoxycarbonyl)(2-(2-(2-(1,3-dioxoisoindolin-2-yl)ethoxy)ethoxy)ethyl)amino)-2-oxopyridin-1(2H)-yl)acetate (2.80 g, 5.02 mmol) in DCM (30 mL) was added a solution of 4 M HCl (gas) in 1,4-dioxane (10 mL) at rt and the mixture was stirred for 1 h at it. On completion, the mixture was concentrated under reduced pressure. The residue was triturated with Et2O and filtered to afford the title compound (2.50 g) as a brown oil. LC/MS (ESI, m/z): [(M+1)]⁺=458.3.

### (S)-1-(isoquinolin-4-yl)piperidine-3-carbonyl chloride (Intermediate D1)

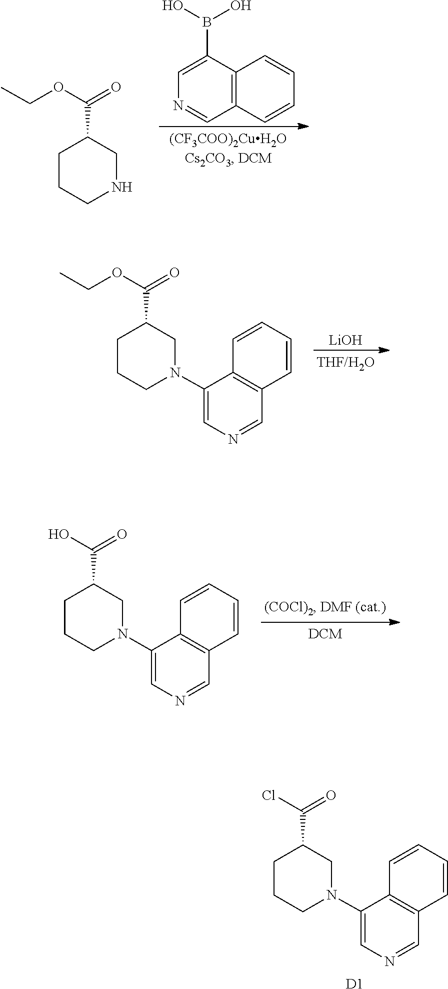

Step 1—Ethyl (S)-1-(isoquinolin-4-yl)piperidine-3-carboxylate. To a stirred solution of ethyl (S)-piperidine-3-carboxylate (30.00 g, 190.8 mmol), isoquinolin-4-ylboronic acid (49.51 g, 286.24 mmol) and Cs2CO3 (186.52 g, 572.47 mmol) in DCM (600 mL) was added Cu(OTf)2 (138.03 g, 381.7 mmol) at rt under air atmosphere. The resulting mixture was stirred for 16 h at rt under air atmosphere. On completion, the mixture was filtered, and the filter cake was washed with DCM (3×200 mL). The filtrate was concentrated under reduced pressure. The residue was purified by silica gel column chromatography, eluted with petroleum ether/EtOAc (38%), and concentrated under reduced pressure to afford the title compound (12 g, 22% yield) as a purple solid. 1H NMR (400 MHz, DMSO-d6) δ 9.02 (s, 1H), 8.20 (s, 1H), 8.13-8.05 (m, 2H), 7.86-7.74 (m, 1H), 7.74-7.61 (m, 1H), 4.12 (q, J=7.2 Hz, 2H), 3.42-3.39 (m, 1H), 3.25-3.16 (m, 1H), 3.13-3.02 (m, 1H), 2.97-2.82 (m, 2H), 2.05-1.95 (m, 1H), 1.95-1.87 (m, 1H), 1.87-1.62 (m, 2H), 1.19 (t, J=7.2 Hz, 3H); LC/MS (ESI, m/z): [(M+1)]⁺=385.2.

Step 2—(S)-1-(isoquinolin-4-yl)piperidine-3-carboxylic acid. To a stirred solution of ethyl (S)-1-(isoquinolin-4-yl)piperidine-3-carboxylate (12 g, 42 mmol) in THF (100 mL) and H2O (100 mL) was added LiOH (5.05 g, 211 mmol) in portions at rt under nitrogen atmosphere. The resulting mixture was then stirred for 16 hr at rt under nitrogen atmosphere. On completion, the mixture was concentrated under vacuum. The residue was purified by reverse phase Flash chromatography (Column: WelFlash™ C18-I, 20-40 m, 330 g; Eluent A: Water (10 mmol/L NH4HCO3); Eluent B: acetonitrile; Gradient: 5%-30% B in 40 min; Flow rate: 80 mL/min; Detector: 220/254 nm; desired fractions were collected at 20% B) and concentrated under reduced pressure to afford the title compound (9 g, 83% yield) as a yellow solid. LC/MS (ESI, m/z): [(M+1)]⁺=257.0.

Step 3—(S)-1-(isoquinolin-4-yl)piperidine-3-carbonyl chloride. To a stirred solution of (S)-1-(isoquinolin-4-yl)piperidine-3-carboxylic acid (5 g, 20 mmol) and oxalic dichloride (4.95 g, 39.0 mmol) in DCM (50 mL) was added DMF (0.148 mL, 1.95 mmol) dropwise at rt under nitrogen atmosphere. The resulting mixture was stirred for 1 hr at rt under nitrogen atmosphere. On completion, the mixture was concentrated under vacuum to give the title compound (4 g, 75% yield) as a yellow solid. LC/MS (ESI, m/z): [(M+MeOH)]⁺=271.2.

### Methyl (S)-2-(5-(N-(2-(2-(2-aminoethoxy)ethoxy)ethyl)-1-(isoquinolin-4-yl)piperidine-3-carboxamido)-2-oxopyridin-1(2H)-yl)acetate (Intermediate E)

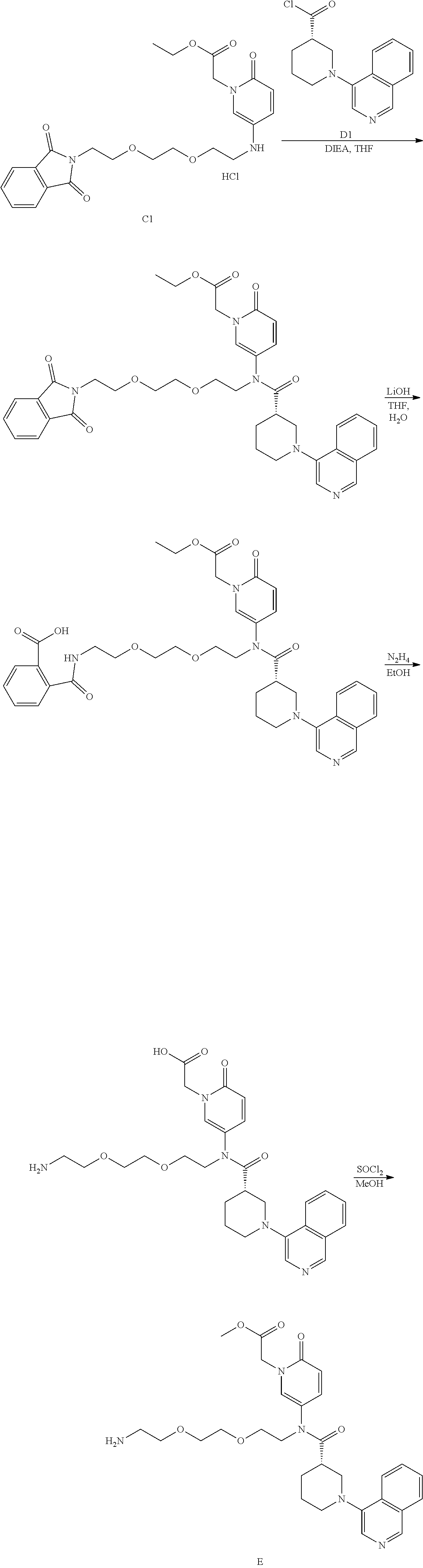

Step 1—Ethyl (S)-2-(5-(N-(2-(2-(2-(1,3-dioxoisoindolin-2-yl)ethoxy)ethoxy)ethyl)-1-(isoquinolin-4-yl)piperidine-3-carboxamido)-2-oxopyridin-1(2H)-yl)acetate. A solution of 2-(5-((2-(2-(2-(1,3-dioxoisoindolin-2-yl)ethoxy)ethoxy)ethyl)amino)-2-oxopyridin-1(2H)-yl)acetate hydrochloride (2.50 g, 5.46 mmol, Intermediate C₁) in THF (30 mL) was treated with DIEA (2.12 g, 16.43 mmol) for 10 min at rt under nitrogen atmosphere. Next, (S)-1-(isoquinolin-4-yl)piperidine-3-carbonyl chloride (2.25 g, 8.21 mmol, Intermediate D1) in THF (10 mL) was added at rt to the reaction mixture. The resulting mixture was then stirred for 1 h at rt. On completion, the mixture was concentrated under reduced pressure. The residue was purified by reverse phase flash chromatography (Column: WelFlash™ C18-I, 20-40 m, 330 g; Eluent A: Water (plus 10 mmol/L NH4HCO3); Eluent B: MeCN; Gradient: 20%-45% B in 20 min; Flow rate: 80 mL/min; Detector: 220/254 nm; desired fractions were collected at 33% B) and concentrated under reduced pressure to afford the title compound (2.30 g, 66% yield) as a yellow green oil. LC/MS (ESI, m/z): [(M+1)]⁺=696.2.

Step 2—(S)-2-((2-(2-(2-(N-(1-(carboxymethyl)-6-oxo-1,6-dihydropyridin-3-yl)-1-(isoquinolin-4-yl)piperidine-3-carboxamido)ethoxy)ethoxy)ethyl)carbamoyl)benzoic acid. To a stirred solution of ethyl (S)-2-(5-(N-(2-(2-(2-(1,3-dioxoisoindolin-2-yl)ethoxy)ethoxy)ethyl)-1-(isoquinolin-4-yl)piperidine-3-carboxamido)-2-oxopyridin-1(2H)-yl)acetate (2.30 g, 3.31 mmol) in THF (10 mL) and H2O (10 mL) was added LiOH (317 mg, 13.22 mmol) at rt and the mixture was stirred for 2 h at rt. On completion, the mixture was acidified to pH 5 with 2M HCl (aq.). The resulting mixture was concentrated under reduced pressure. The residue was purified by reverse phase flash chromatography (Column: WelFlash™ C18-I, 20-40 m, 120 g; Eluent A: Water (plus 10 mmol/L FA); Eluent B: MeCN; Gradient: 10%-30% B in 25 min; Flow rate: 80 mL/min; Detector: 220/254 nm; desired fractions were collected at 22% B) and concentrated under reduced pressure to afford the title compound (1.70 g, 75% yield) as a yellow oil. LC/MS (ESI, m/z): [(M+1)]⁺=686.3.

Step 3—(S)-2-(5-(N-(2-(2-(2-aminoethoxy)ethoxy)ethyl)-1-(isoquinolin-4-yl)piperidine-3-carboxamido)-2-oxopyridin-1(2H)-yl)acetic acid. To a stirred solution of (S)-2-((2-(2-(2-(N-(1-(carboxymethyl)-6-oxo-1,6-dihydropyridin-3-yl)-1-(isoquinolin-4-yl)piperidine-3-carboxamido)ethoxy)ethoxy)ethyl)carbamoyl)benzoic acid (800 mg, 1.17 mmol) in EtOH (10 mL) was added hydrazine hydrate (1.70 g, 35.0 mmol) at rt. The resulting mixture was stirred overnight at 80° C. On completion, the mixture was cooled to rt and concentrated under reduced pressure. The residue was purified by reverse phase flash chromatography (Column: WelFlash™ C18-I, 20-40 m, 330 g; Eluent A: Water (plus 10 mmol/L NH4HCO3); Eluent B: MeCN; Gradient: 15%-35% B in 20 min; Flow rate: 80 mL/min; Detector: 220/254 nm; desired fractions were collected at 26% B) and concentrated under reduced pressure to afford the title compound (560 mg, 89% yield) as a yellow solid. LC/MS (ESI, m/z): [(M+1)]⁺=538.3.

Step 4—Methyl (S)-2-(5-(N-(2-(2-(2-aminoethoxy)ethoxy)ethyl)-1-(isoquinolin-4-yl)piperidine-3-carboxamido)-2-oxopyridin-1(2H)-yl)acetate. To a stirred solution of (S)-2-(5-(N-(2-(2-(2-aminoethoxy)ethoxy)ethyl)-1-(isoquinolin-4-yl)piperidine-3-carboxamido)-2-oxopyridin-1(2H)-yl)acetic acid (540 mg, 1.00 mmol) in MeOH (5 mL) was added SOCl2 (358 mg, 3.01 mmol) at rt. The resulting mixture was stirred for 1 h at 70° C. On completion, the mixture was cooled to rt and concentrated under reduced pressure to give the title compound (500 mg) as a yellow solid. LC/MS (ESI, m/z): [(M+1)]+=552.3.

### 6-((4-((2-methoxy-3-(pyrimidin-2-yl)phenyl)amino)-5-(methylcarbamoyl)pyridin-2-yl)amino)nicotinic acid (Intermediate F1)

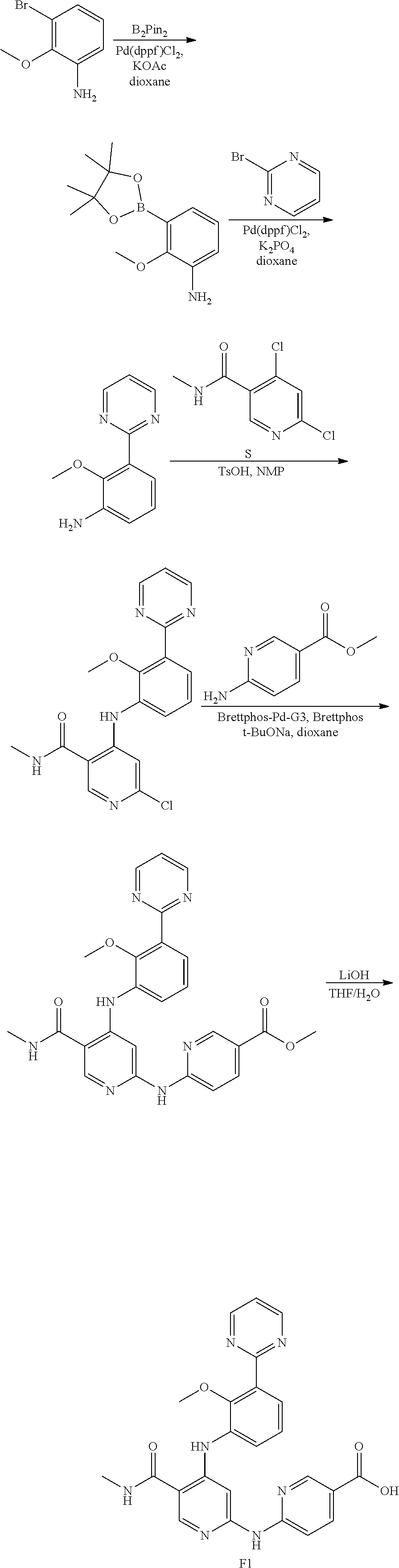

Step 1—2-Methoxy-3-(4,4,5,5-tetramethyl-1,3,2-dioxaborolan-2-yl)aniline. A mixture of 4,4,5,5-tetramethyl-2-(4,4,5,5-tetramethyl-1,3,2-dioxaborolan-2-yl)-1,3,2-dioxaborolane (145.16 g, 571.65 mmol, CAS #73183-34-3), 3-bromo-2-methoxy-aniline (105 g, 519 mmol), Pd(dppf)Cl2 (38.03 g, 51.97 mmol), and KOAc (102.00 g, 1.04 mol) in dioxane (350 mL) was degassed and purged with N2 three times. Then the mixture was stirred at 100° C., for 16 hr under N2 atmosphere. On completion, the reaction was poured into ice-water (300 mL) and stirred for 5 min. The aqueous phase was extracted with ethyl acetate (200 mL×3). The combined organic phase was washed with brine (300 mL), dried with anhydrous Na2SO₄, filtered and concentrated in vacuo. The residue was purified by silica gel chromatography (column height: 250 mm, diameter: 100 mm, 100-200 mesh silica gel, Petroleum ether/Ethyl acetate=10/1, 5/1) to afford the title compound (60 g, 46% yield) as white solid. 1H NMR (400 MHz, DMSO-d6) δ=9.54 (s, 1H), 7.28-7.13 (m, 3H), 6.92-6.83 (m, 2H), 6.72-6.59 (m, 1H), 6.82-6.58 (m, 2H), 4.79 (s, 2H), 3.82-3.69 (m, 5H), 2.86 (t, J=6.7 Hz, 2H).

Step 2—2-Methoxy-3-pyrimidin-2-yl-aniline. To a stirred solution of 2-methoxy-3-(4,4,5,5-tetramethyl-1,3,2-dioxaborolan-2-yl) aniline (16 g, 64 mmol) and 2-bromopyrimidine (9.19 g, 57.8 mmol) in dioxane (1600 mL) and H2O (320 mL) were added K3PO4 (27.27 g, 128.5 mmol) and Pd(dppf) Cl2 (4.70 g, 6.42 mmol) in turns at 25° C. under nitrogen atmosphere. The resulting mixture was stirred for 16 h at 80° C. under nitrogen atmosphere. On completion, the mixture was filtered and concentrated in vacuo. The residue was purified by silica gel chromatography (column height: 250 mm, diameter: 100 mm, 100-200 mesh silica gel, Petroleum ether/Ethyl acetate=2/1, 1/1) to afford the title compound (10 g, 76% yield) as yellow solid. 1H NMR (400 MHz, CHLOROFORM-d) 6=8.54 (s, 1H), 7.48-7.33 (m, 1H), 6.58 (br s, 1H), 3.00 (d, J=4.9 Hz, 3H).

Step 3—6-chloro-4-((2-methoxy-3-(pyrimidin-2-yl)phenyl)amino)-N-methylnicotinamide. To a solution of 2-methoxy-3-pyrimidin-2-yl-aniline (6.5 g, 32.3 mmol) and 4,6-dichloro-N-methyl-pyridine-3-carboxamide (6.6 g, 32.3 mmol, Intermediate S) in THF (120 mL) was dropwise added LiHMDS (1 M in THF, 80.7 mL) at 15° C., then the solution was stirred at 15° C., for 2 h. On completion, the residue was poured into aq. NH4Cl (100 mL) and stirred for 5 min. The aqueous phase was then extracted with ethyl acetate (40 mL×3). The combined organic phase was washed with brine (40 mL), dried with anhydrous Na2SO₄, filtered and concentrated in vacuo. To the residue was added EA (40 mL) and stirred the mixture was stirred for 5 min. Then the solution was filtered, the filter cake was collected and dried to give the title compound (7.2 g) as a brown solid. LC/MS (ESI, m/z): [(M+1)]⁺=370.1.

Step 4—Methyl 6-[[4-(2-methoxy-3-pyrimidin-2-yl-anilino)-5-(methylcarbamoyl)-2-pyridyl]amino]pyridine-3-carboxylate. A mixture of 6-chloro-4-(2-methoxy-3-pyrimidin-2-yl-anilino)-N-methyl-pyridine-3-carboxamide (6 g, 20 mmol), methyl 6-aminopyridine-3-carboxylate (2.47 g, 16.2 mmol, CAS #36052-24-1), BrettPhos Pd G3 (1.47 g, 1.62 mmol, CAS #1470372-59-8), Cs2CO3 (10.57 g, 32.45 mmol) and BrettPhos (1.74 g, 3.24 mmol, CAS #1070663-78-3) in dioxane (60 mL) was degassed and purged with N2 three times. Then the mixture was stirred at 85° C., for 16 hr under N2 atmosphere. On completion, the mixture was filtered and concentrated in vacuo. The residue was purified by silica gel chromatography (column height: 250 mm, diameter: 100 mm, 100-200 mesh silica gel, Petroleum ether/Ethyl acetate=2/1, 1/1) to afford the title compound (4.7 g, 7.4 mmol, 45% yield) as a yellow solid. LC/MS (ESI, m/z): [(M+1)]⁺=486.2.

Step 5—6-[[4-(2-Methoxy-3-pyrimidin-2-yl-anilino)-5-(methylcarbamoyl)-2-pyridyl]amino]pyridine-3-carboxylic acid. To a stirred solution of methyl 6-[[4-(2-methoxy-3-pyrimidin-2-yl-anilino)-5-(methylcarbamoyl)-2-pyridyl]amino]pyridine-3-carboxylate (3 g, 6 mmol) in THF (30 mL) and H2O (30 mL) was added LiOH (1.48 g 61.8 mmol) at 25° C. under nitrogen atmosphere. The resulting mixture was stirred for 2 h at 25° C. under nitrogen atmosphere. On completion, the mixture was filtered and concentrated in vacuo. The crude product was triturated with EA at 25° C., for 10 min, then filtered to give the title compound (2.6 g) as a brown solid. LC/MS (ESI, m/z): [(M+1)]⁺=472.2; 1H NMR (400 MHz, DMSO-d6) δ=12.04 (br s, 1H), 10.98 (s, 1H), 9.28 (br d, J=2.6 Hz, 1H), 8.96 (d, J=4.9 Hz, 2H), 8.83 (s, 1H), 8.62 (s, 1H), 8.25 (dd, J=1.6, 8.6 Hz, 1H), 7.65 (brt, J=6.8 Hz, 2H), 7.52 (t, J=4.8 Hz, 1H), 7.41-7.33 (m, 1H), 7.24 (br d, J=8.6 Hz, 1H), 7.08-6.96 (m, 1H), 3.70 (s, 3H), 2.82 (br d, J=4.1 Hz, 3H).

### Methyl (S)-2-(5-(N-(2-(2-(2-(2-aminoethoxy)ethoxy)ethoxy)ethyl)-1-(isoquinolin-4-yl)piperidine-3-carboxamido)-2-oxopyridin-1(2H)-yl)acetate (Intermediate GI)

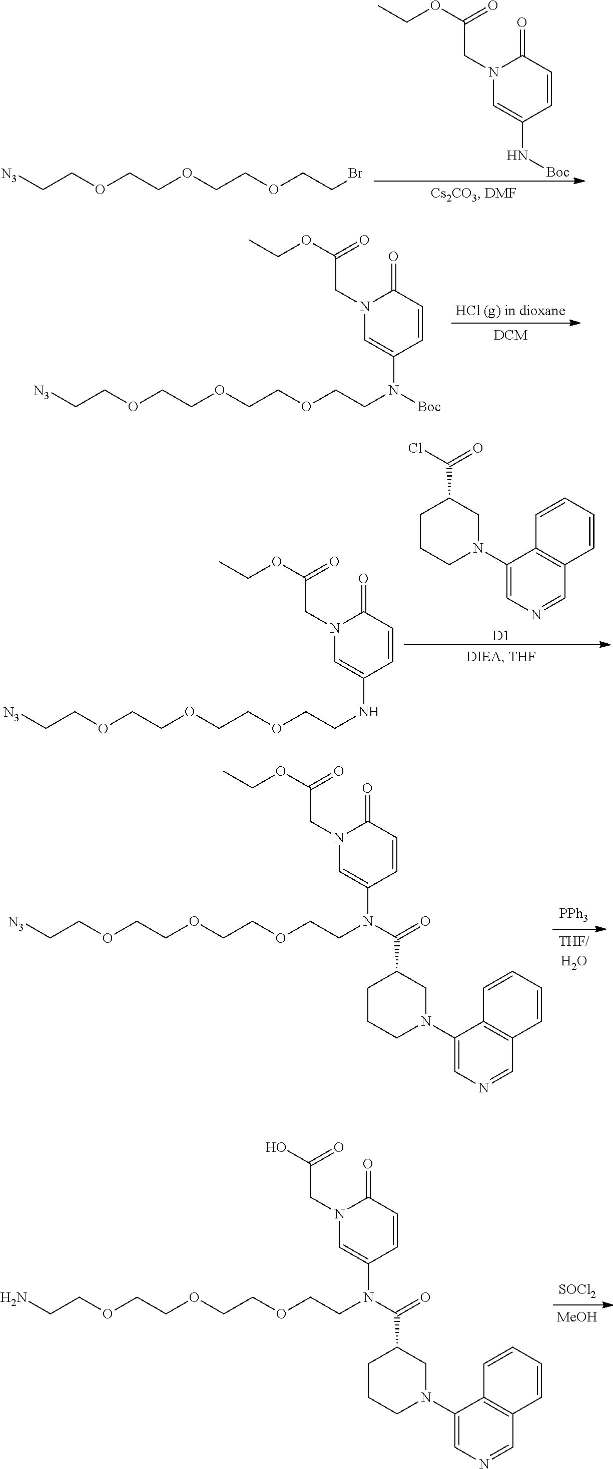

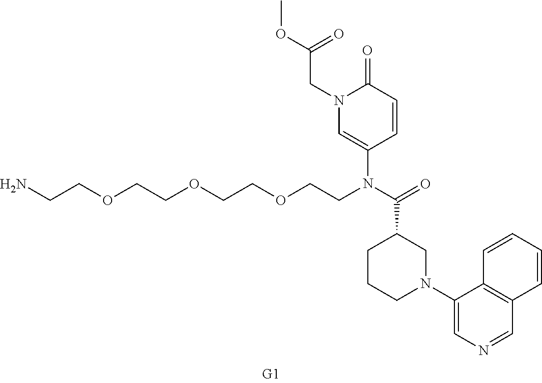 [SMILES: COC(=O)Cn1cc(N(CCOCCOCCOCCN)C(=O)[C@H]2CCCN(c3cncc4ccccc34)C2)ccc1=O]

Step 1—Ethyl 2-(5-((2-(2-(2-(2-azidoethoxy)ethoxy)ethoxy)ethyl)(tert-butoxycarbonyl)amino)-2-oxopyridin-1(2H)-yl)acetate. A mixture of 1-azido-2-(2-(2-(2-bromoethoxy)ethoxy)ethoxy)ethane (4.00 g, 14.2 mmol, CAS #1446282-43-4), ethyl 2-(5-((tert-butoxycarbonyl)amino)-2-oxopyridin-1(2H)-yl)acetate (3.50 g, 11.8 mmol, synthesized via Steps 1-2 of Intermediate A1) and Cs2CO3 (11.55 g, 35.44 mmol) in DMF (60 mL) was stirred for 16 hr at 50° C. On completion, the mixture was cooled to rt and purified directly by reversed-phase flash chromatography (Column: Spherical C18, 20-40 m, 330 g; Mobile Phase A: Water (plus 0.1% HCOOH), Mobile Phase B: acetonitrile; Flow rate: 80 mL/min; Gradient (B %): 5%˜30%, 4 min; 30%˜50%, 20 min; 50%˜95%; 2 min; 95%, 5 min; Detector: 254 nm; the fractions containing desired product were collected at 40% B) and concentrated under reduced pressure to afford the title compound (4 g, 68% yield) as a green oil. 1H NMR (400 MHz, CDCl3) δ 7.49-7.33 (m, 2H), 6.54 (d, J=9.6 Hz, 1H), 4.64 (s, 2H), 4.24 (q, J=7.2 Hz, 2H), 3.76-3.54 (m, 14H), 3.41-3.37 (m, 2H), 1.44 (s, 9H), 1.30 (t, J=7.2 Hz, 3H); LC/MS (ESI, m/z): [(M+1)]⁺=498.2.

Step 2—Ethyl 2-(5-((2-(2-(2-(2-azidoethoxy)ethoxy)ethoxy)ethyl)amino)-2-oxopyridin-1(2H)-yl)acetate. To a stirred mixture of ethyl 2-(5-((2-(2-(2-(2-azidoethoxy)ethoxy)ethoxy)ethyl)(tert-butoxycarbonyl)amino)-2-oxopyridin-1(2H)-yl)acetate (4 g, 8 mmol) in DCM (20 mL) was added HCl (gas) in 1,4-dioxane (20 mL) dropwise at rt. The resulting mixture was stirred for 1 h at rt. On completion, the mixture was concentrated under reduced pressure to afford the title compound (2.8 g, 88% yield) as a green oil. 1H NMR (400 MHz, DMSO-d6) δ 7.72 (s, 1H), 7.62-7.57 (m, 1H), 6.53 (dd, J=9.6, 1.6 Hz, 1H), 4.71 (s, 2H), 4.16 (q, J=7.2 Hz, 2H), 3.66 (t, J=5.2 Hz, 2H), 3.64-3.51 (m, 12H), 3.44-3.37 (m, 2H), 1.22 (t, J=7.2 Hz, 3H). LC/MS (ESI, m/z): [(M+1)]⁺=398.2.

Step 3—Ethyl (S)-2-E(5-(N-(2-(2-(2-(2-azidoethoxy)ethoxy)ethoxy)ethyl)-1-(isoquinolin-4-yl)piperidine-3-carboxamido)-2-oxopyridin-1(2H)-yl)acetate. To a stirred mixture of ethyl 2-(5-((2-(2-(2-(2-azidoethoxy)ethoxy)ethoxy)ethyl)amino)-2-oxopyridin-1(2H)-yl)acetate (2.00 g, 5.03 mmol) and (S)-1-(isoquinolin-4-yl)piperidine-3-carbonyl chloride (1.66 g, 6.04 mmol, Intermediate D1) in THF (30 mL) was added DIEA (1.95 g, 15.10 mmol) dropwise at 0° C. The resulting mixture was then stirred for 1 h at rt. On completion, the mixture was purified by reversed-phase flash chromatography (Column: Spherical C18, 20˜40 m, 330 g; Mobile Phase A: Water (plus 0.1% HCOOH), Mobile Phase B: acetonitrile; Flow rate: 80 mL/min; Gradient (B %): 5%˜20%, 4 min; 20%˜40%, 20 min; 40%˜95%; 2 min; 95%, 5 min; Detector: 254 nm; the fractions containing desired product were collected at 27% B) and concentrated under reduced pressure to afford the title compound (1.20 g, 38% yield) as a green oil. LC/MS (ESI, m/z): [(M+1)]+=636.3.

Step 4—(S)-2-(5-(N-(2-(2-(2-(2-aminoethoxy)ethoxy)ethoxy)ethyl)-1-(isoquinolin-4-yl)piperidine-3-carboxamido)-2-oxopyridin-1(2H)-yl)acetic acid. A mixture of ethyl (S)-2-(5-(N-(2-(2-(2-(2-azidoethoxy)ethoxy)ethoxy)ethyl)-1-(isoquinolin-4-yl)piperidine-3-carboxamido)-2-oxopyridin-1(2H)-yl)acetate (1.10 g, 1.73 mmol) and PPh3 (908 mg, 3.46 mmol) in THF (16 mL) and H2O (4 mL) was stirred for 16 hr at 50° C. The mixture was cooled to rt and purified by reversed-phase flash chromatography (Column: Spherical C18, 20˜40 μm, 330 g; Mobile Phase A: Water (plus 0.1% HCOOH), Mobile Phase B: acetonitrile; Flow rate: 80 mL/min; Gradient (B %): 5%˜10%, 4 min; 10%˜20%, 20 min; 20%˜95%; 2 min; 95%, 5 min; Detector: 254 nm; the fractions containing desired product were collected at 14% B) and concentrated under reduced pressure to afford the title compound (600 mg, 60% yield) as a yellow oil. LC/MS (ESI, m/z): [(M+1)]⁺=582.3.

Step 5—Methyl (S)-2-(5-(N-(2-(2-(2-(2-aminoethoxy)ethoxy)ethoxy)ethyl)-1-(isoquinolin-4-yl)piperidine-3-carboxamido)-2-oxopyridin-1(2H)-yl)acetate. To a stirred solution of (S)-2-(5-(N-(2-(2-(2-(2-aminoethoxy)ethoxy)ethoxy)ethyl)-1-(isoquinolin-4-yl)piperidine-3-carboxamido)-2-oxopyridin-1(2H)-yl)acetic acid (450 mg, 0.77 mmol) in MeOH (10 mL) was added SOCl2 (368 mg, 3.09 mmol) dropwise at rt under air atmosphere. The resulting mixture was stirred for additional 1 h at 70° C. The mixture was allowed to cool down to rt and was concentrated under reduced pressure to give the title compound.

### 2-(14-Bromo-3,6,9,12-tetraoxatetradecyl)isoindoline-1,3-dione (Intermediate H1)

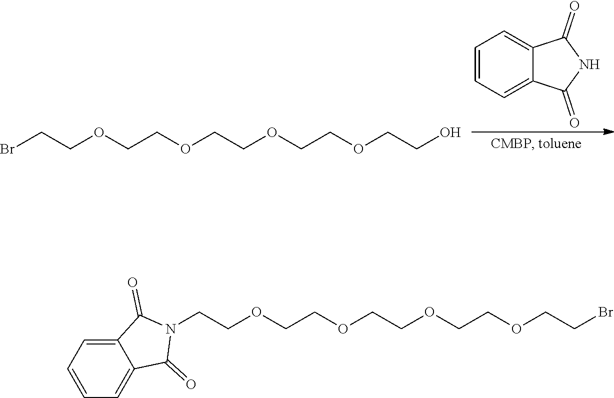 [SMILES: O=C1NC(=O)c2ccccc21 ; OCCOCCOCCOCCOCCBr ; O=C1c2ccccc2C(=O)N1CCOCCOCCOCCOCCBr]

Step 1—2-(14-Bromo-3,6,9,12-tetraoxatetradecyl)isoindoline-1,3-dione. To a stirred solution of 14-bromo-3,6,9,12-tetraoxatetradecan-1-ol (4.00 g, 13.3 mmol) and phthalimide (1.95 g, 13.3 mmol) in toluene (60 mL) was added 2-(tributyl-phosphanylidene)acetonitrile (4.81 g, 19.9 mmol) at rt under nitrogen atmosphere. The resulting mixture was stirred for 2 h at 110° C. under nitrogen atmosphere. On completion, the mixture was cooled to rt and concentrated under reduced pressure. The residue was purified by silica gel column chromatography, eluted with petroleum ether/EtOAc (1:1), to afford the title compound (4.10 g, 71% yield) as a yellow oil. LC/MS (ESI, m/z): [(M+1)]⁺=430.1, 432.1.

### Ethyl 2-(5-((tert-butoxycarbonyl)(14-(1,3-dioxoisoindolin-2-yl)-3,6,9,12-tetraoxatetradecyl)amino)-2-oxopyridin-1(2H)-yl)acetate (Intermediate I)

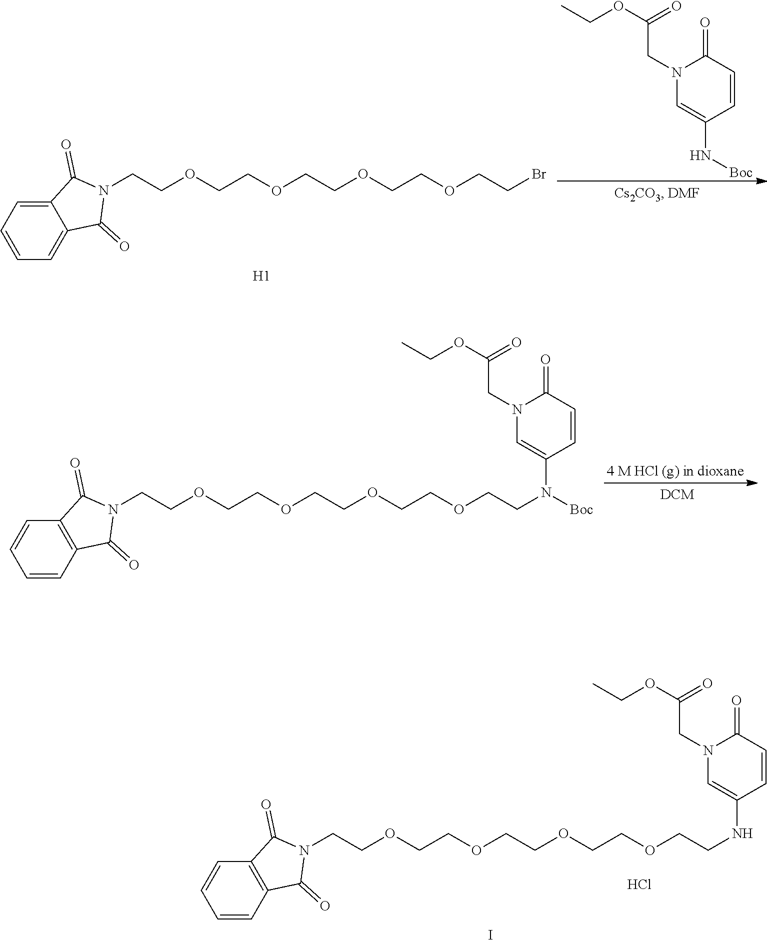

Step 1—Ethyl 2-(5-((tert-butoxycarbonyl)(14-(1,3-dioxoisoindolin-2-yl)-3,6,9,12-tetraoxatetradecyl)amino)-2-oxopyridin-1(2H)-yl)acetate. To a stirred solution of 2-(14-bromo-3,6,9,12-tetraoxatetradecan-1-yl)isoindole-1,3-dione (6.00 g, 13.94 mmol, Intermediate H) and ethyl 2-{5-[(tert-butoxycarbonyl)amino]-2-oxopyridin-1-yl}acetate (4.13 g, 13.94 mmol, synthesized via Steps 1-2 of Intermediate A) in DMF (60 mL) was added Cs2CO3 (9.09 g, 27.88 mmol) in portions at rt under nitrogen atmosphere. The resulting mixture was stirred for 4 h at 70° C. under nitrogen atmosphere. On completion, the mixture was cooled to rt and concentrated under vacuum. The residue was purified by reverse phase Flash chromatography (Column: WelFlash™ C18-I, 20-40 μm, 120 g; Eluent A: Water (10 mmol/L NH4HCO3); Eluent B: MeCN; Gradient: 25%-55% B in 40 min; Flow rate: 80 mL/min; Detector: 220/254 nm; desired fractions were collected at 40% B) and concentrated under reduced pressure to afford the title compound (1.40 g, 15% yield) as a yellow oil. LC/MS (ESI, m/z): [(M+1)]⁺=646.3.

Step 2—Ethyl 2-(5-((14-(1,3-dioxoisoindolin-2-yl)-3,6,9,12-tetraoxatetradecyl)amino)-2-oxopyridin-1(2H)-yl)acetate hydrochloride. To a stirred solution of ethyl 2-(5-((tert-butoxycarbonyl)(14-(1,3-dioxoisoindolin-2-yl)-3,6,9,12-tetraoxatetradecyl)amino)-2-oxopyridin-l(2H)-yl)acetate (1.40 g, 2.16 mmol) in DCM (10 mL) was added 4 M HCl (gas) in 1,4-dioxane (10 mL) at rt under nitrogen atmosphere. The resulting mixture was stirred for 1 h at rt under nitrogen atmosphere. On completion, the mixture was concentrated under reduced pressure. The residue was purified by trituration with Et2O (10 mL) to afford the title compound (1.2 g, 95% yield) as a yellow oil. LC/MS (ESI, m/z): [(M+1)]⁺=546.2.

### Methyl (S)-2-(5-(N-(14-amino-3,6,9,12-tetraoxatetradecyl)-1-(isoquinolin-4-yl)piperidine-3-carboxamido)-2-oxopyridin-1(2H)-yl)acetate (Intermediate J)

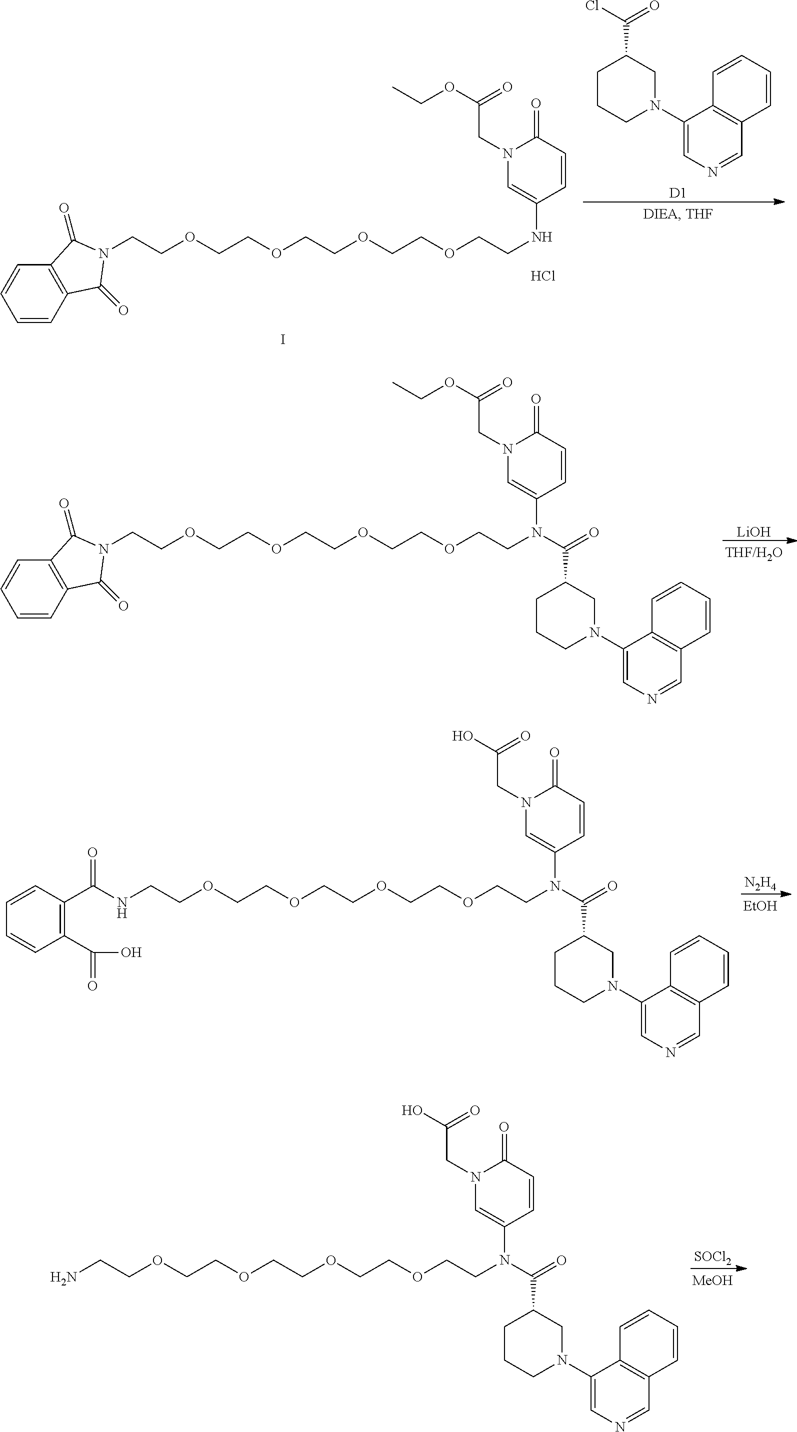

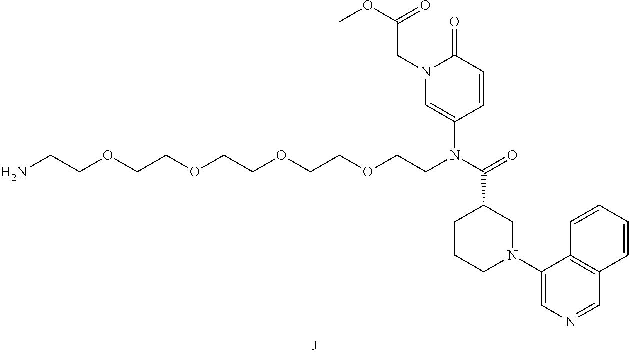 [SMILES: COC(=O)Cn1cc(N(CCOCCOCCOCCOCCN)C(=O)[C@H]2CCCN(c3cncc4ccccc34)C2)ccc1=O]

Step 1—Ethyl (S)-2-(5-(N-(14-(1,3-dioxoisoindolin-2-yl)-3,6,9,12-tetraoxatetradecyl)-1-(isoquinolin-4-yl)piperidine-3-carboxamido)-2-oxopyridin-1(2H)-yl)acetate. To a stirred solution of ethyl 2-(5-((14-(1,3-dioxoisoindolin-2-yl)-3,6,9,12-tetraoxatetradecyl)amino)-2-oxopyridin-1(2H)-yl)acetate hydrochloride (700 mg, 1.20 mmol, Intermediate I) and (3S)-1-(isoquinolin-4-yl)piperidine-3-carbonyl chloride (661 mg, 2.40 mmol, Intermediate D1) in THF (15 mL) was added DIEA (466 mg, 3.60 mmol) at rt under nitrogen atmosphere. The resulting mixture was stirred for 1 h at rt under nitrogen atmosphere. On completion, the mixture was concentrated under vacuum. The residue was purified by reverse phase flash chromatography (Column: WelFlash™ C18-I, 20-40 m, 120 g; Eluent A: Water (10 mmol/L NH4HCO3); Eluent B: MeCN; Gradient: 20%-50% B in 40 min; Flow rate: 80 mL/min; Detector: 220/254 nm; desired fractions were collected at 35% B) and concentrated under reduced pressure to afford the title compound (400 mg, 42% yield) as a yellow oil. LC/MS (ESI, m/z): [(M+1)]⁺=784.4.

Step 2—(S)-2-((2-(1-(carboxymethyl)-6-oxo-1,6-dihydropyridin-3-yl)-1-(1-(isoquinolin-4-yl)piperidin-3-yl)-1-oxo-5,8,11,14-tetraoxa-2-azahexadecan-16-yl)carbamoyl)benzoic acid. To a stirred solution of ethyl (S)-2-(5-(N-(14-(1,3-dioxoisoindolin-2-yl)-3,6,9,12-tetraoxatetradecyl)-1-(isoquinolin-4-yl)piperidine-3-carboxamido)-2-oxopyridin-1(2H)-yl)acetate (450 mg, 0.57 mmol) in THF (5 mL) and H2O (5 mL) was added LiOH (55 mg, 2.29 mmol) at rt under nitrogen atmosphere. The resulting mixture was stirred for 1 h rt under nitrogen atmosphere. On completion, the mixture was acidified to pH 3 with 2 M HCl (aq.). The resulting mixture was concentrated under vacuum. The residue was purified by reverse phase flash chromatography (Column: WelFlash™ C18-I, 20-40 μm, 120 g; Eluent A: Water (10 mmol/L NH4HCO3); Eluent B: MeCN; Gradient: 15%-50% B in 40 min; Flow rate: 80 mL/min; Detector: 220/254 nm; desired fractions were collected at 25% B) and concentrated under reduced pressure to afford the title compound (400 mg, 90% yield) as a yellow solid. LC/MS (ESI, m/z): [(M+1)]⁺=774.3.

Step 3—(S)-2-(5-(N-(14-amino-3,6,9,12-tetraoxatetradecyl)-1-(isoquinolin-4-yl)piperidine-3-carboxamido)-2-oxopyridin-1(2H)-yl)acetic acid. To a stirred solution of (S)-2-((2-(1-(carboxymethyl)-6-oxo-1,6-dihydropyridin-3-yl)-1-(1-(isoquinolin-4-yl)piperidin-3-yl)-1-oxo-5,8,11,14-tetraoxa-2-azahexadecan-16-yl)carbamoyl)benzoic acid (400 mg, 0.51 mmol) in EtOH (8 mL) was added hydrazine hydrate (457 mg, 7.75 mmol, 85% solution) at rt under nitrogen atmosphere. The resulting mixture was stirred for 16 h at 80° C. under nitrogen atmosphere. On completion, the mixture was cooled to rt and concentrated under reduced pressure. The residue was purified by reverse phase flash chromatography (Column: WelFlash™ C18-I, 20-40 m, 120 g; Eluent A: Water (10 mmol/L NH4HCO3); Eluent B: MeCN; Gradient: 5%-30% B in 40 min; Flow rate: 45 mL/min; Detector: 220/254 nm; desired fractions were collected at 17% B) and concentrated under reduced pressure to afford the title compound (300 mg, 92% yield) as a yellow solid. LC/MS (ESI, m/z): [(M+1)]⁺=626.3.

Step 4—Methyl (S)-2-(5-(N-(14-amino-3,6,9,12-tetraoxatetradecyl)-1-(isoquinolin-4-yl)piperidine-3-carboxamido)-2-oxopyridin-1(2H)-yl)acetate. To a stirred solution of (S)-2-(5-(N-(14-amino-3,6,9,12-tetraoxatetradecyl)-1-(isoquinolin-4-yl)piperidine-3-carboxamido)-2-oxopyridin-1(2H)-yl)acetic acid (300 mg, 0.47 mmol) in MeOH (6 mL) was added SOCl2 (171 mg, 1.43 mmol) at rt under nitrogen atmosphere. The resulting mixture was stirred for 1 h at 70° C. under nitrogen atmosphere. On completion, the mixture was cooled to rt and concentrated under reduced pressure to afford the title compound (300 mg, 97% yield) as a yellow solid. LC/MS (ESI, m/z): [(M+1)]⁺=640.3.

### Ethyl 2-(5-((8-(1,3-dioxoisoindolin-2-yl)octyl)amino)-2-oxopyridin-1(2H)-yl)acetate hydrochloride (Intermediate K)

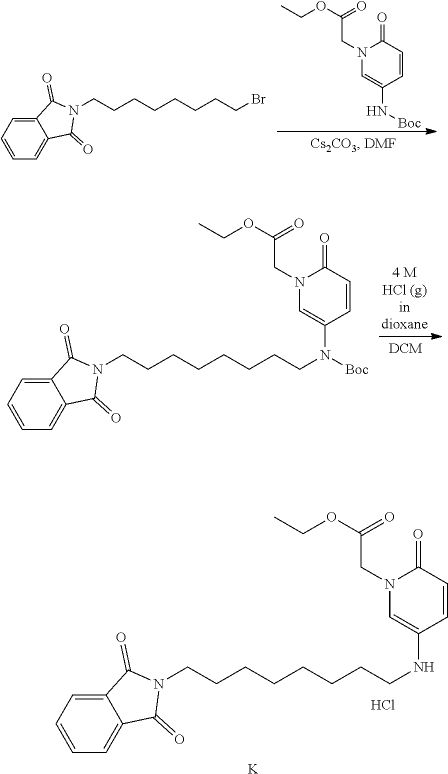

Step 1—Ethyl 2-(5-((tert-butoxycarbonyl)(8-(1,3-dioxoisoindolin-2-yl)octyl)amino)-2-oxopyridin-1(2H)-yl)acetate. To a stirred solution of 2-(8-bromooctyl)isoindoline-1,3-dione (9.10 g, 26.9 mmol, CAS #17702-83-9) and ethyl 2-(5-((tert-butoxycarbonyl)amino)-2-oxopyridin-1(2H)-yl)acetate (3.99 g, 13.5 mmol, synthesized via Steps 1-2 of Intermediate A1) in DMF (20 mL) was added Cs2CO3 (13.15 g, 40.36 mmol) at rt. The resulting mixture was stirred overnight at 50° C. On completion, the mixture was cooled to rt and diluted with H2O (50 mL). The resulting mixture was extracted with EtOAc (3×30 mL), then the combined organic layers were dried over anhydrous Na2SO₄. After filtration, the filtrate was concentrated under reduced pressure. The residue was purified by silica gel column chromatography, eluted with petroleum ether/EtOAc (1:2). The resulting mixture was concentrated under reduced pressure. The residue was purified by reverse phase flash chromatography (Column: WelFlash™ C18-I, 20-40 m, 330 g; Eluent A: Water (plus 10 mmol/L NH4HCO3); Eluent B: MeCN; Gradient: 45%-65% B in 35 min; Flow rate: 80 mL/min; Detector: 220/254 nm; desired fractions were collected at 65% B) and concentrated under reduced pressure to afford the title compound (4.30 g, 58% yield) as a brown oil. LC/MS (ESI, m/z): [(M+1)]⁺=554.3.

Step 2—Ethyl 2-(5-((8-(1,3-dioxoisoindolin-2-yl)octyl)amino)-2-oxopyridin-1(2H)-yl)acetate hydrochloride. To a stirred solution of ethyl 2-(5-((tert-butoxycarbonyl)(8-(1,3-dioxoisoindolin-2-yl)octyl)amino)-2-oxopyridin-1(2H)-yl)acetate (4.30 g, 7.77 mmol) in DCM (20 mL) was added a solution of 4 M HCl (gas) in 1,4-dioxane (20 mL) at rt and the mixture was stirred for 2 h at rt. On completion, the mixture was concentrated under reduced pressure. The residue was triturated with Et2O to afford the title compound (3.50 g, 92% yield) as a yellow solid. LC/MS (ESI, m/z): [(M+1)]⁺=454.2.

### Methyl (S)-2-(5-(N-(8-aminooctyl)-1-(isoquinolin-4-yl)piperidine-3-carboxamido)-2-oxopyridin-1(2H)-yl)acetate (Intermediate L)

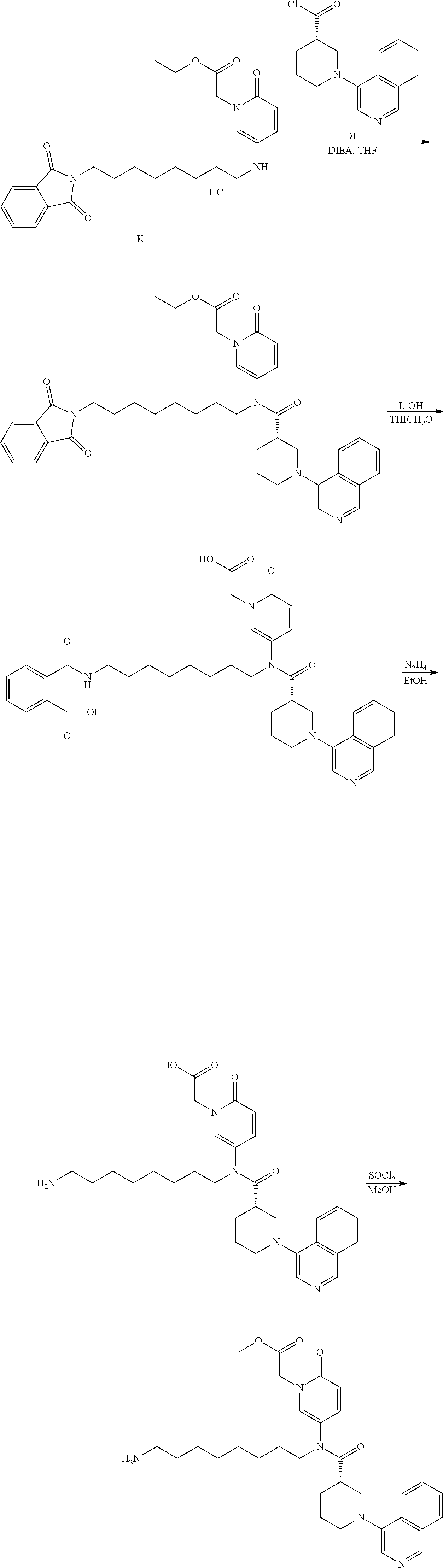

Step 1—Ethyl (S)-2-(5-(N-(8-(1,3-dioxoisoindolin-2-yl)octyl)-1-(isoquinolin-4-yl)piperidine-3-carboxamido)-2-oxopyridin-1(2H)-yl)acetate. A solution of ethyl 2-(5-((8-(1,3-dioxoisoindolin-2-yl)octyl)amino)-2-oxopyridin-1(2H)-yl)acetate hydrochloride (2.00 g, 4.41 mmol, Intermediate K) in THE (10 mL) was treated with DIEA (1.71 g, 13.23 mmol) for 10 min at rt under nitrogen atmosphere, followed by the addition of (S)-1-(isoquinolin-4-yl)piperidine-3-carbonyl chloride (2.42 g, 8.82 mmol, Intermediate D1) at 0° C. The resulting mixture was stirred for 1 h at rt. On completion, the mixture was concentrated under reduced pressure. The residue was purified by reverse phase flash chromatography (Column: WelFlash™ C18-I, 20-40 m, 330 g; Eluent A: Water (plus 10 mmol/L NH4HCO3); Eluent B: MeCN; Gradient: 45%-75% B in 20 min; Flow rate: 80 mL/min; Detector: 220/254 nm; desired fractions were collected at 65% B) and concentrated under reduced pressure to afford the title compound (800 mg, 26% yield) as a yellow green oil. LC/MS (ESI, m/z): [(M+1)]⁺=692.5.

Step 2—(S)-2-(5-(N-(8-(1,3-dioxoisoindolin-2-yl)octyl)-1-(isoquinolin-4-yl)piperidine-3-carboxamido)-2-oxopyridin-1(2H)-yl)acetic acid. To a stirred solution of ethyl (S)-2-(5-(N-(8-(1,3-dioxoisoindolin-2-yl)octyl)-1-(isoquinolin-4-yl)piperidine-3-carboxamido)-2-oxopyridin-1(2H)-yl)acetate (800 mg, 1.16 mmol) in THF (3 mL) and H2O (3 mL) was added LiOH (55 mg, 2.31 mmol) at rt and the mixture was stirred for 1 h at rt. On completion, the mixture was acidified to pH 5 with 2M HCl (aq.). The resulting mixture was concentrated under reduced pressure. The residue was purified by reverse phase flash chromatography (Column: WelFlash™ C18-I, 20-40 m, 330 g; Eluent A: Water (plus 10 mmol/L FA); Eluent B: MeCN; Gradient: 20%-40% B in 20 min; Flow rate: 80 mL/min; Detector: 220/254 nm; desired fractions were collected at 35% B) and concentrated under reduced pressure to afford the title compound (680 mg, 86% yield) as a yellow solid. LC/MS (ESI, m/z): [(M+1)]⁺=682.3.

Step 3—(S)-2-(5-(N-(8-aminooctyl)-1-(isoquinolin-4-yl)piperidine-3-carboxamido)-2-oxopyridin-1(2H)-yl)acetic acid. To a stirred solution of (S)-2-(5-(N-(8-(1,3-dioxoisoindolin-2-yl)octyl)-1-(isoquinolin-4-yl)piperidine-3-carboxamido)-2-oxopyridin-1(2H)-yl)acetic acid (680 mg, 1.00 mmol) in EtOH (10 mL) was added hydrazine hydrate (1.50 g, 29.91 mmol) at rt. The resulting mixture was then stirred overnight at 80° C. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH4HCO3); Eluent B: MeCN; Gradient: 20%-40% B in 20 min; Flow rate: 80 mL/min; Detector: 220/254 nm; desired fractions were collected at 30% B) and concentrated under reduced pressure to afford the title compound (340 mg, 64% yield) as a yellow solid. LC/MS (ESI, m/z): [(M+1)]⁺=534.4.

Step 4—Methyl (S)-2-(5-(N-(8-aminooctyl)-1-(isoquinolin-4-yl)piperidine-3-carboxamido)-2-oxopyridin-1(2H)-yl)acetate. To a stirred solution of (S)-2-(5-(N-(8-aminooctyl)-1-(isoquinolin-4-yl)piperidine-3-carboxamido)-2-oxopyridin-1(2H)-yl)acetic acid (340 mg, 0.64 mmol) in MeOH (5 mL) was added SOCl2 (227 mg, 1.91 mmol) at rt. Then the resulting mixture was stirred for 1 h at 70° C. On completion, the mixture was concentrated under reduced pressure to give the title compound (300 mg) as a yellow solid. LC/MS (ESI, m/z): [(M+1)]⁺=548.4.

### 2-(11-Bromoundecyl)isoindoline-1,3-dione (Intermediate M)

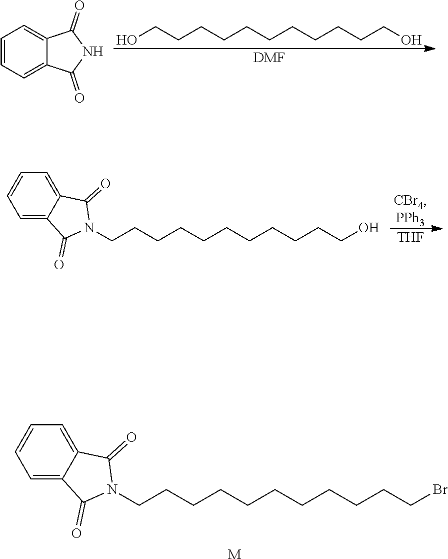

Step 1—2-(11-Hydroxyundecyl)isoindoline-1,3-dione. A mixture of isoindoline-1,3-dione (11 g, 60 mmol) and undecane-1,11-diol (14.92 g, 59.39 mmol) in DMF (60 mL) was stirred for 2 h at rt. On completion, the mixture was diluted with water (180 mL) and extracted with EtOAc (3×100 mL). The combined organic layers were washed with brine (3×200 mL), and dried over anhydrous Na2SO₄. The resulting mixture was concentrated under reduced pressure. The residue was purified by silica gel column chromatography, eluted with CH2Cl2/EA (10:1), to afford the title compound (10 g, 46%) as a white solid. 1H NMR (400 MHz, DMSO-d6) δ 7.91-7.77 (m, 4H), 4.30 (t, J=5.2 Hz, 1H), 3.55 (t, J=7.2 Hz, 2H), 3.42-3.29 (m, 2H), 1.63-1.50 (m, 2H), 1.43-1.32 (m, 2H), 1.31-1.14 (m, 14H); LC/MS (ESI, m/z): [(M+1)]⁺=318.4.

Step 2—2-(11-Bromoundecyl)isoindoline-1,3-dione. To a stirred solution of 2-(11-hydroxyundecyl)isoindoline-1,3-dione (10.0 g, 31.5 mmol) and PPh3 (12 g, 47.26 mmol) in THF (100 mL) was added CBr4 (16 g, 47 mmol) in portions at 0° C. under air atmosphere. The resulting mixture was then stirred for 2 hr at rt. On completion, the mixture was concentrated under reduced pressure. The residue was purified by silica gel column chromatography, eluted with CH2C2/EA (15:1), to afford the title compound (10.5 g, 88% yield) as a white solid. 1H NMR (300 MHz, CDCl3) δ 7.93-7.81 (m, 2H), 7.78-7.67 (m, 2H), 3.75-3.64 (m, 2H), 3.42 (t, J=6.8 Hz, 2H), 1.87 (p, J=6.8 Hz, 2H), 1.76-1.63 (m, 2H), 1.50-1.24 (m, 14H); LC/MS (ESI, m/z): [(M+1)]⁺=380.1.

### Ethyl 2-(5-((11-(1,3-dioxoisoindolin-2-yl)undecyl)amino)-2-oxopyridin-1(2H)-yl)acetate (Intermediate N)

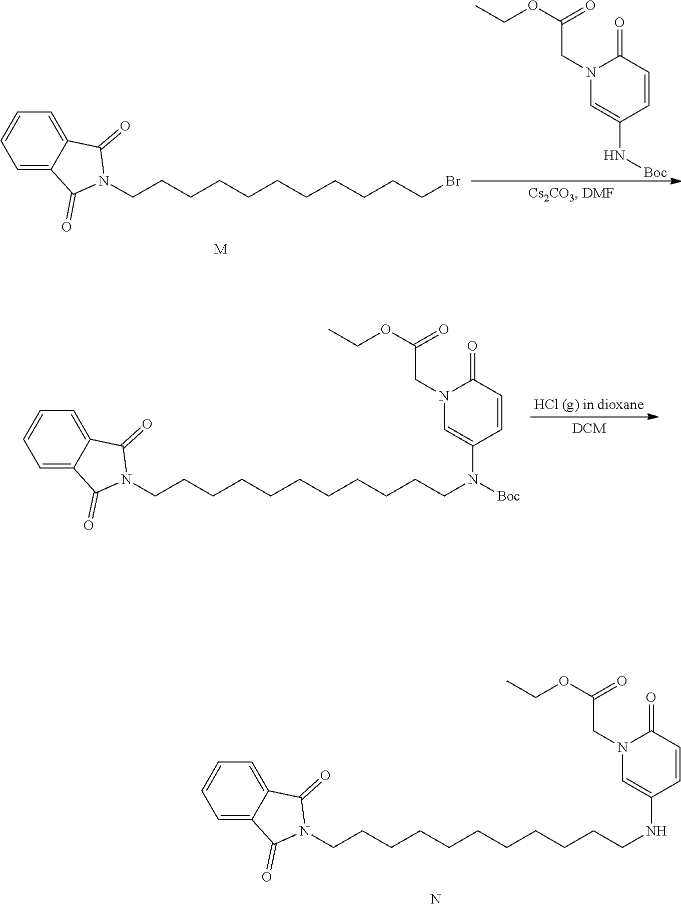

Step 1—Ethyl 2-(5-((tert-butoxvcarbonyl)(11-(1,3-dioxoisoindolin-2-yl)undecyl)amino)-2-oxopyridin-1(2H)-yl)acetate

To a stirred solution of 2-(11-bromoundecyl)isoindoline-1,3-dione (4.62 g, 12.15 mmol, Intermediate M) and ethyl 2-(5-((tert-butoxycarbonyl)amino)-2-oxopyridin-1(2H)-yl)acetate (3.00 g, 10.1 mmol, synthesized via Steps 1-2 of Intermediate A1) in DMF (45 mL) was added Cs2CO3 (10.56 g, 32.40 mmol) in portions at rt under nitrogen atmosphere. The resulting mixture was then stirred for 16 hr at 50° C. On completion, the mixture was concentrated under reduced pressure. The crude product was purified by reverse phase flash chromatography (column: WelFlash™ C18-I, 20-40 μm, 330 g; Eluent A: Water (plus 10 mmol/L FA); Eluent B: acetonitrile; Gradient: 50%-80% B in 25 min; Flow rate: 80 mL/min; Detector: 220/254 nm; desired fractions were collected at 72% B) and concentrated under reduced pressure to afford the title compound (4.8 g, 75%) as a brown solid. 1H NMR (400 MHz, CDCl3) δ 7.88-7.82 (m, 2H), 7.76-7.69 (m, 2H), 7.28-7.21 (m, 1H), 7.14 (s, 1H), 6.59 (d, J=9.6 Hz, 1H), 4.64 (s, 2H), 4.25 (q, J=7.2 Hz, 2H), 3.68 (t, J=7.2 Hz, 2H), 3.55-3.41 (m, 2H), 2.02 (s, 3H), 1.73-1.61 (m, 2H), 1.56-1.49 (m, 2H), 1.44 (s, 9H), 1.36-1.19 (m, 14H); LC/MS (ESI, m/z): [(M+1)]⁺=596.3.

Step 2—Ethyl 2-(5-((11-(1,3-dioxoisoindolin-2-yl)undecyl)amino)-2-oxopyridin-1(2H)-yl)acetate hydrochloride. To a stirred solution of ethyl 2-(5-((tert-butoxycarbonyl)(11-(1,3-dioxoisoindolin-2-yl)undecyl)amino)-2-oxopyridin-1(2H)-yl)acetate (2.3 g, 3.9 mmol) in DCM (30 mL) was added HCl (gas) in 1,4-dioxane (30 mL) in portions at rt under air atmosphere. The resulting mixture was then stirred for 1 h at rt. On completion, the mixture was concentrated under reduced pressure to give the title compound (1.9 g, 98% yield) as a yellow solid. 1H NMR (400 MHz, CDCl3) δ 7.94-7.80 (m, 3H), 7.73-7.71 (m, 2H), 7.57 (dd, J=9.6, 2.8 Hz, 1H), 6.66 (d, J=9.6 Hz, 1H), 4.65 (s, 2H), 4.25 (q, J=7.2 Hz, 2H), 3.72 (s, 2H), 3.68 (t, J=7.2 Hz, 3H), 3.24-3.15 (m, 2H), 1.91-1.79 (m, 2H), 1.73-1.62 (m, 2H), 1.38-1.21 (m, 14H); LC/MS (ESI, m/z): [(M+1)]⁺=496.2.

### (S)-2-(5-(N-(11-aminoundecyl)-1-(isoquinolin-4-yl)piperidine-3-carboxamido)-2-oxopyridin-1(2H)-yl)acetic acid (Intermediate O)

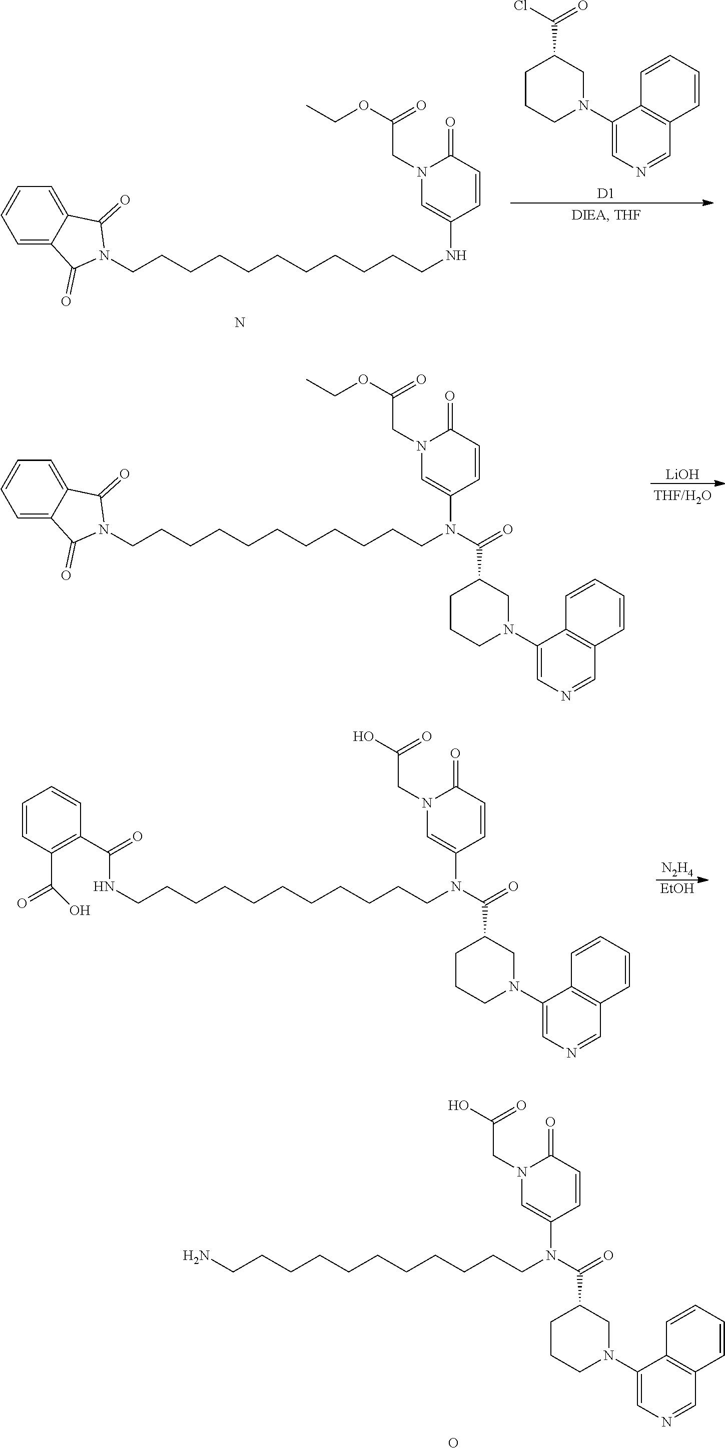

Step 1—Ethyl (S)-2-(5-(N-(11-(1,3-dioxoisoindolin-2-yl)undecyl)-1-(isoquinolin-4-yl)piperidine-3-carboxamido)-2-oxopyridin-1(2H)-yl)acetate. To a stirred solution of ethyl 2-(5-((11-(1,3-dioxoisoindolin-2-yl)undecyl)amino)-2-oxopyridin-1(2H)-yl)acetate hydrochloride (2.0 g, 4.0 mmol, Intermediate N) in THE (15 mL) was added DIEA (1.56 g, 12.11 mmol) at 0° C. under air atmosphere. The resulting mixture was then stirred for additional 30 min at rt. Next, (S)-1-(isoquinolin-4-yl)piperidine-3-carbonyl chloride (3.33 g, 12.1 mmol, Intermediate D1) was added in THF (5 mL) dropwise at 0° C. The resulting mixture was then stirred for additional 1 hr at rt. On completion, the reaction mixture was concentrated under reduced pressure. The residue was purified by silica gel column chromatography, eluted with CH2Cl2/MeOH (10:1), to afford the title compound (1.3 g, 33%) as a dark green solid. 1H NMR (400 MHz, CDCl3) δ 8.97 (s, 1H), 8.19 (s, 1H), 8.01-7.89 (m, 2H), 7.89-7.81 (m, 2H), 7.76-7.56 (m, 4H), 7.26-7.14 (m, 2H), 6.68 (d, J=9.6 Hz, 111), 4.68-4.50 (m, 1H), 4.31-4.04 (m, 2H), 3.75-3.45 (m, 5H), 3.44-3.26 (m, 2H), 3.22-3.01 (m, 111), 3.04-2.73 (m, 2H), 1.96-1.76 (m, 4H), 1.74-1.60 (m, 3H), 1.58-1.46 (m, 2H), 1.37-1.16 (m, 16H); LC/MS (ESI, m/z): [(M+1)]⁺=734.3.

Step 2—(S)-2-((11-(N-(1-(carboxymethyl)-6-oxo-1,6-dihydropyridin-3-yl)-1-(isoquinolin-4-yl)piperidine-3-carboxamido)undecyl)carbamoyl)benzoic acid. To a stirred solution of ethyl (S)-2-(5-(N-(11-(1,3-dioxoisoindolin-2-yl)undecyl)-1-(isoquinolin-4-yl)piperidine-3-carboxamido)-2-oxopyridin-1(2H)-yl)acetate (1.3 g, 1.77 mmol) and LiOH (170 mg, 7.08 mmol) in THF (10 mL) was added H2O (10 mL) at rt under air atmosphere. The resulting mixture was stirred for 1 h at rt. On completion, the mixture was concentrated under reduced pressure. The mixture was acidified to pH 5 with HCl (aq.). The precipitated solids were collected by filtration and washed with water (3×5 mL), then dried under reduced pressure to give the title compound (1.2 g, 74% yield) as a dark green solid. 1H NMR (400 MHz, DMSO-d6) δ 8.98 (s, 1H), 8.41-8.27 (m, 1H), 8.13 (s, 1H), 8.08 (d, J=8.0 Hz, 1H), 7.87 (d, J=2.8 Hz, 1H), 7.81-7.63 (m, 4H), 7.58-7.44 (m, 2H), 7.39 (dd, J=7.2, 1.6 Hz, 1H), 6.51 (d, J=9.6 Hz, 1H), 4.79-4.45 (m, 3H), 3.28 (t, J=13.2 Hz, 2H), 3.20-3.07 (m, 3H), 2.97-2.73 (m, 3H), 1.82-1.74 (m, 1H), 1.72-1.55 (m, 1H), 1.53-1.36 (m, 4H), 1.31-1.14 (m, 16H); LC/MS (ESI, m/z): [(M+1)]⁺=724.3.

Step 3—(S)-2-(5-(N-(11-aminoundecyl)-1-(isoquinolin-4-yl)piperidine-3-carboxamido)-2-oxopyridin-1(2H)-yl)acetic acid. To a stirred solution of (S)-2-((11-(N-(1-(carboxymethyl)-6-oxo-1,6-dihydropyridin-3-yl)-1-(isoquinolin-4-yl)piperidine-3-carboxamido)undecyl)carbamoyl)benzoic acid (1.1 g, 1.5 mmol) in EtOH (15 mL) was added NH2NH2·H2O (2.21 mL, 45.6 mmol) dropwise at rt under air atmosphere. The resulting mixture was stirred for 16 hr at 80° C. On completion, the mixture was concentrated under reduced pressure. The residue was purified by reverse phase flash chromatography (Column: WelFlash™ C18-I, 20-40 μm, 330 g; Eluent A: Water (plus 10 mmol/L NH4HCO3); Eluent B: ACN; Gradient: 25%-55% B in 25 min; Flow rate: 80 mL/min; Detector: 220 nm; desired fractions were collected at 40% B) and concentrated under reduced pressure to afford the title compound (500 mg, 57%) as a light brown solid. 1H NMR (300 MHz, DMSO-d6) δ 8.98 (s, 1H), 8.46 (s, 3H), 8.12 (s, 1H), 8.12-8.02 (m, 1H), 7.80-7.59 (m, 4H), 7.41 (d, J=9.2 Hz, 1H), 6.43 (d, J=9.6 Hz, 1H), 4.49-4.35 (m, 2H), 3.38-3.21 (m, 3H), 2.96-2.65 (m, 5H), 1.95-1.84 (m, 1H), 1.82-1.49 (m, 4H), 1.47-1.37 (m, 2H), 1.33-1.13 (m, 16H); LC/MS (ESI, m/z): [(M-1)]−=574.2.

### 2-(14-Bromotetradecyl)isoindole-1,3-dione (Intermediate P)

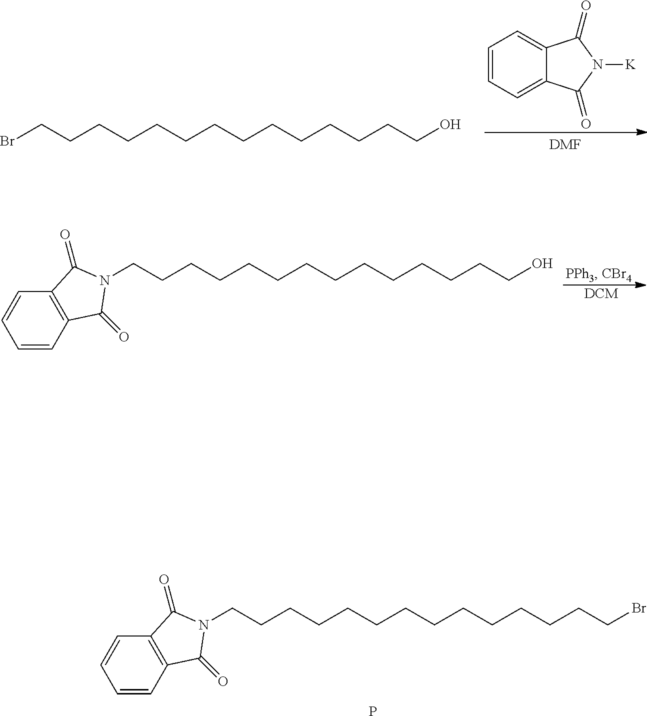

Step 1—2-(14-Hydroxytetradecyl)isoindole-1,3-dione. To a stirred solution of 14-bromotetradecan-1-ol (5 g, 17.0 mmol, CAS #72995-94-9) in DMF (100 mL) was added phthalimide (3.76 g, 25.5 mmol) at rt. The resulting mixture was stirred for 16 h at 70° C. under nitrogen atmosphere. On completion, the reaction mixture was cooled to rt and diluted with water (300 mL), then extracted with EtOAc (3×150 mL). The combined organic layers were washed with water (3×100 mL), and dried over anhydrous Na2SO₄. After filtration, the filtrate was concentrated under reduced pressure to afford the title compound (6.4 g) as a white solid. LC/MS (ESI, m/z): [(M+1)]⁺=360.2; 1H NMR (400 MHz, Chloroform-d) δ 7.86 (dd, J=5.4, 3.1 Hz, 2H), 7.73 (dd, J=5.5, 3.0 Hz, 2H), 3.73-3.64 (m, 4H), 1.72-1.51 (m, 4H), 1.40-1.21 (m, 20H).

Step 2—2-(14-Bromotetradecyl)isoindole-1,3-dione. To a stirred solution of 2-(14-hydroxytetradecyl)isoindole-1,3-dione (6.4 g) and PPh3 (7.00 g, 26.7 mmol) in DCM (120 mL) was added CBr4 (8.86 g, 26.7 mmol) at rt under nitrogen atmosphere. The resulting mixture was stirred for 1 h at rt under nitrogen atmosphere. On completion, the mixture was concentrated under reduced pressure. The residue was purified by silica gel column chromatography, eluted with PE/EA (9:1), to afford the title compound (6 g, 80% yield) as a white solid. LC/MS (ESI, m/z): [(M+1)]⁺=422.2; 1H NMR (400 MHz, Chloroform-d) δ 7.86 (dd, J=5.4, 3.1 Hz, 2H), 7.72 (dd, J=5.4, 3.1 Hz, 2H), 3.69 (t, J=7.0 Hz, 2H), 3.42 (t, J=6.9 Hz, 2H), 1.91-1.82 (m, 2H), 1.73-1.62 (m, 2H), 1.47-1.21 (m, 20H).

### Ethyl 2-(5-((14-(1,3-dioxoisoindolin-2-yl)tetradecyl)amino)-2-oxopyridin-1(2H)-yl)acetate (Intermediate Q)

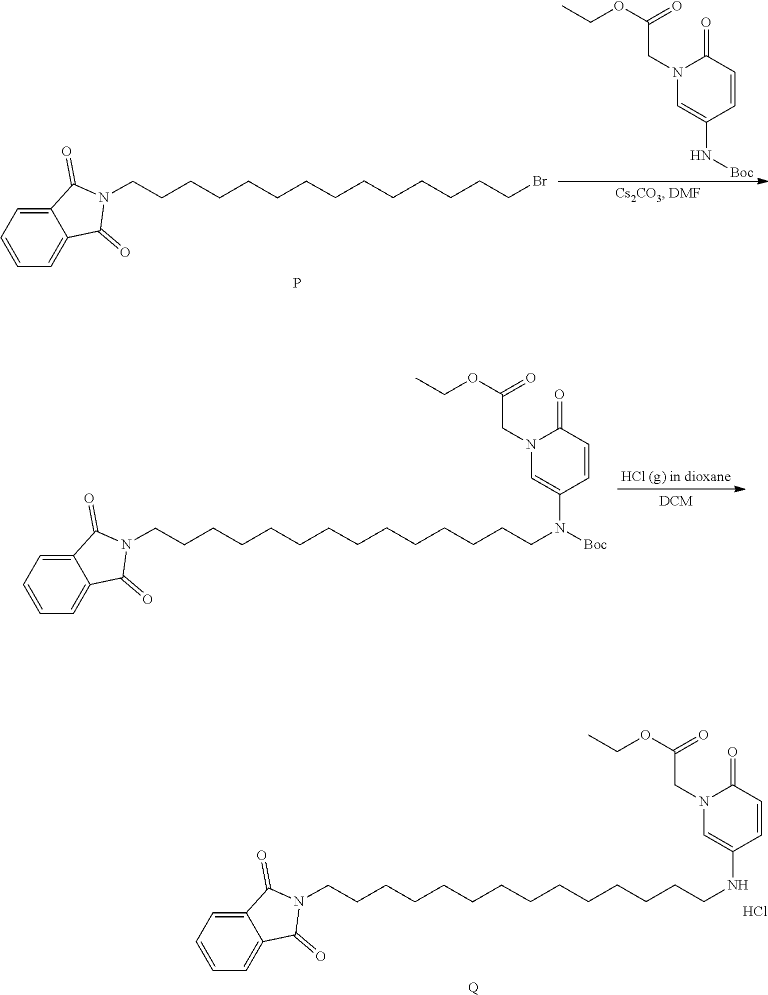

Step 1—Ethyl 2-(5-((tert-butoxycarbonyl)(14-(1,3-dioxoisoindolin-2-yl)tetradecyl)amino)-2-oxopyridin-1(2H)-yl)acetate. To a stirred solution of 2-(14-bromotetradecyl)isoindoline-1,3-dione (2.57 g, 6.07 mmol, Intermediate P) and Cs2CO3 (4.95 g, 15.19 mmol) in DMF (45 mL) was added ethyl 2-(5-((tert-butoxycarbonyl)amino)-2-oxopyridin-1(2H)-yl)acetate (1.50 g, 5.06 mmol, synthesized via Steps 1-2 of Intermediate A1) at rt under nitrogen atmosphere. The resulting mixture was stirred for additional 16 hr at 50° C. On completion, the mixture was extracted with EtOAc (3×100 mL). The combined organic layers were washed with brine (3×200 mL), and dried over anhydrous Na2SO₄. After filtration, the filtrate was concentrated under reduced pressure. The residue was purified by silica gel column chromatography, eluted with PE/EA (50%), to afford the title compound (2.8 g, 81% yield) as a dark green solid. 1H NMR (400 MHz, CDCl3) δ 7.85 (dd. J=5.4, 3.2 Hz, 2H), 7.72 (dd, J=5.4, 3.2 Hz, 2H), 7.24 (s, 1H), 7.14 (s, 1H), 6.59 (d, J=9.6 Hz, 1H), 4.64 (s, 2H), 4.26 (q, J=7.2 Hz, 2H), 3.73-3.64 (m, 3H), 3.53-3.42 (m, 2H), 1.73-1.61 (m, 2H), 1.58-1.50 (m, 1H), 1.44 (s, 9H), 1.38-1.20 (m, 23H). LC/MS (ESI, m/z): [(M+1)]+=638.3.

Step 2—Ethyl 2-(5-((14-(1,3-dioxoisoindolin-2-yl)tetradecyl)amino)-2-oxopyridin-1(2H)-yl)acetate hydrochloride. To a stirred solution of ethyl 2-(5-((tert-butoxycarbonyl)(14-(1,3-dioxoisoindolin-2-yl)tetradecyl)amino)-2-oxopyridin-1(2H)-yl)acetate (2.8 g 4.39 mmol) in DCM (20 mL) was added HCl (gas) in 1,4-dioxane (20 mL) at rt. The resulting mixture was then stirred for 1 hr at rt. On completion, the mixture was concentrated under reduced pressure to give the title compound (2.5 g, 89% yield) as a yellow solid. 1H NMR (400 MHz, CDCl3) δ 7.89-7.84 (m, 3H), 7.72 (dd, J=5.4, 3.2 Hz, 2H), 7.56 (dd, J=10.0, 2.8 Hz, 1H), 6.66 (d, J=10.0 Hz, 1H), 4.64 (s, 2H), 4.25 (q, J=7.2 Hz, 2H), 3.71-3.65 (m, 2H), 3.27-3.13 (m, 2H), 1.92-1.79 (m, 2H), 1.73-1.61 (m, 2H), 1.40-1.19 (m, 24H); LC/MS (ESI, m/z): [(M+1)]⁺=574.3.

### Methyl (S)-2-(5-(N-(14-aminotetradecyl)-1-(isoquinolin-4-yl)piperidine-3-carboxamido)-2-oxopyridin-1(2H)-yl)acetate (Intermediate R)

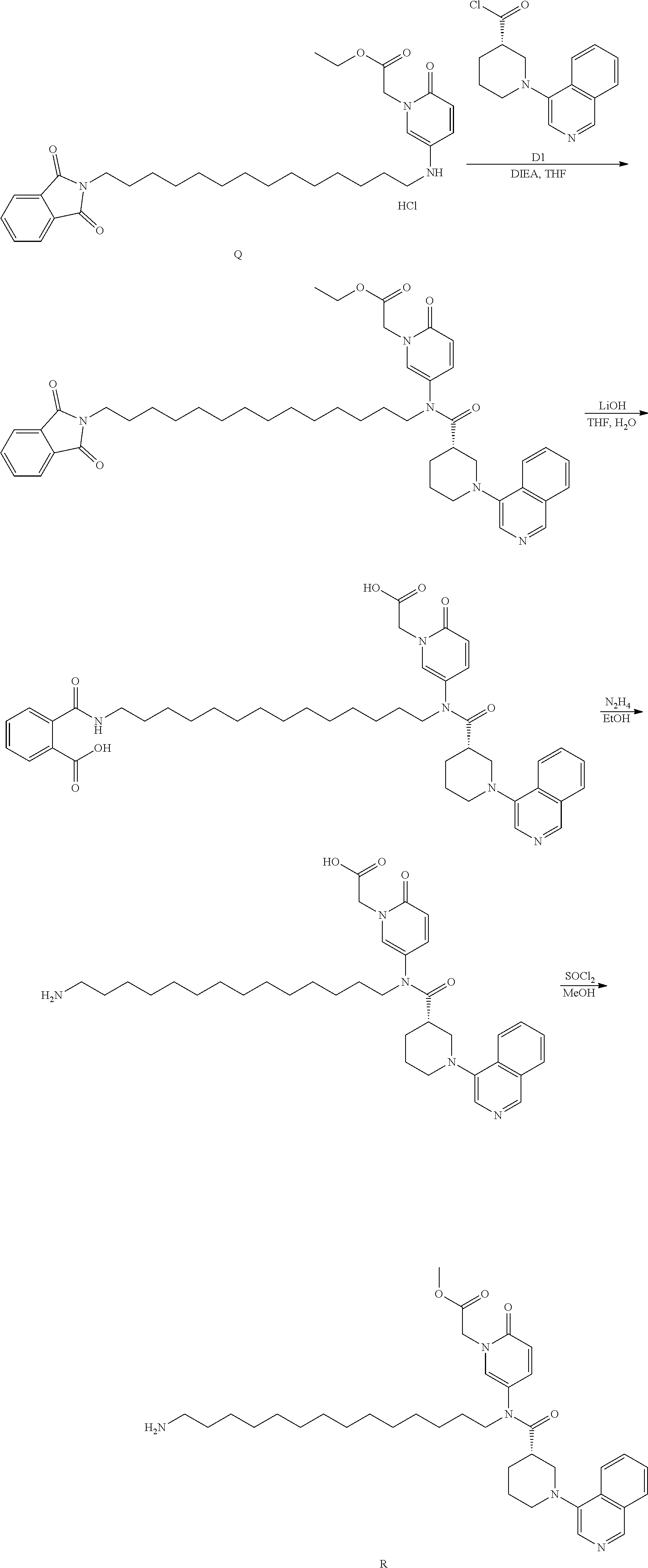

Step 1—Ethyl (S)-2-(5-(N-(14-(1,3-dioxoisoindolin-2-yl)tetradecyl)-1-(isoquinolin-4-yl)piperidine-3-carboxamido)-2-oxopyridin-1(2H)-yl)acetate. A stirred solution of ethyl 2-(5-((14-(1,3-dioxoisoindolin-2-yl)tetradecyl)amino)-2-oxopyridin-1(2H)-yl)acetate hydrochloride (1.30 g, 2.42 mmol, Intermediate Q) and DIEA (1.68 mL, 9.67 mmol) in THF (20 mL) was stirred for 30 min at 0° C. under air atmosphere. Next, (S)-1-(isoquinolin-4-yl)piperidine-3-carbonyl chloride (1.00 g, 3.63 mmol, Intermediate D1) was added at 0° C., to the mixture. The resulting mixture was then stirred for 1 hr at rt. On completion, the mixture was concentrated under vacuum. The crude product was purified by silica gel column chromatography, eluted with CH2Cl2/MeOH (10:1), to afford the title compound (1.6 g, 85% yield) as a dark green solid. 1H NMR (400 MHz, DMSO-d6) δ 8.99 (s, 1H), 8.18-8.02 (m, 2H), 7.98-7.44 (m, 9H), 6.54 (d, J=9.6 Hz, 1H), 4.91-4.51 (m, 2H), 4.23-4.08 (m, 2H), 3.55 (t, J=7.2 Hz, 2H), 3.32-3.18 (m, 3H), 2.96-2.69 (m, 4H), 1.94-1.73 (m, 2H), 1.71-1.52 (m, 4H), 1.47-1.32 (m, 2H), 1.31-1.03 (m, 23H). LC/MS (ESI, m/z): [(M+1)]⁺=776.4.

Step 2—(S)-2-((14-(N-(1-(carboxymethyl)-6-oxo-1,6-dihydropyridin-3-yl)-1-(isoquinolin-4-yl)piperidine-3-carboxamido)tetradecyl)carbamoyl)benzoic acid. To a stirred solution of ethyl (S)-2-(5-(N-(14-(1,3-dioxoisoindolin-2-yl)tetradecyl)-1-(isoquinolin-4-yl)piperidine-3-carboxamido)-2-oxopyridin-1(2H)-yl)acetate (1.6 g, 2.1 mmol) in THF (20 mL) and H2O (20 mL) was added LiOH (197 mg, 8.25 mmol) at rt. The resulting mixture was stirred for 1 hr at rt. On completion, the mixture was acidified to pH 5 with aq HCl. The mixture was then purified by reversed-phase flash chromatography (column, C18 silica gel; mobile phase, MeCN in Water (10 mmol/L NH4HCO3), 20% to 60% gradient in 30 min; detector, UV 254 nm) to afford the title compound (1 g, 63% yield) as a light green solid. LC/MS (ESI, m/z): [(M+1)]+=766.4.

Step 3—(S)-2-(5-(N-(14-aminotetradecyl)-1-(isoquinolin-4-yl)piperidine-3-carboxamido)-2-oxopyridin-1(2H)-yl)acetic acid. To a stirred solution of (S)-2-((14-(N-(1-(carboxymethyl)-6-oxo-1,6-dihydropyridin-3-yl)-1-(isoquinolin-4-yl)piperidine-3-carboxamido)tetradecyl)carbamoyl)benzoic acid (1 g, 1.31 mmol) in EtOH (10 mL) was added hydrazine hydrate (1.96 g, 39.18 mmol) at rt. The resulting mixture was stirred for 16 hr at 80° C. On completion, the mixture was concentrated under vacuum. The crude product was purified by reversed-phase flash chromatography (column, C18 silica gel; mobile phase, MeCN in Water (10 mmol/L NH4HCO3), 20% to 50% gradient in 30 min; detector, UV 254 nm) to afford the title compound (500 mg, 62% yield) as a light yellow solid. 1H NMR (400 MHz, DMSO-d6) δ 8.98 (s, 1H), 8.12 (s, 1H), 8.07 (d, J=8.0 Hz, 1H), 7.96 (s, 1H), 7.79-7.59 (m, 3H), 7.40 (s, 1H), 6.43 (d, J=9.6 Hz, 1H), 4.44-4.34 (m, 2H), 3.41-3.19 (m, 7H), 2.78 (d, J=11.2 Hz, 1H), 2.73-2.65 (m, 1H), 1.96-1.85 (m, 1H), 1.80-1.57 (m, 1H), 1.55-1.47 (m, 1H), 1.45-1.36 (m, 2H), 1.29-1.14 (m, 26H). LC/MS (ESI, m/z): [(M+1)]⁺=618.4.

Step 4—Methyl (S)-2-(5-(N-(14-aminotetradecyl)-1-(isoquinolin-4-yl)piperidine-3-carboxamido)-2-oxopyridin-1(2H)-yl)acetate. To a stirred solution of (S)-2-(5-(N-(14-aminotetradecyl)-1-(isoquinolin-4-yl)piperidine-3-carboxamido)-2-oxopyridin-1(2H)-yl)acetic acid (500 mg, 0.8 mmol) in MeOH (7 mL) was added SOCl2 (385 mg, 3.24 mmol) at rt. The resulting mixture was then stirred for 1 h at 70° C. On completion, the mixture was cooled to rt and concentrated under vacuum to afford the title compound (500 mg, 98% yield) as a yellow solid. LC/MS (ESI, m/z): [(M+1)]⁺=632.4.

### 4,6-Dichloro-N-methyl-pyridine-3-carboxamide (Intermediate S)

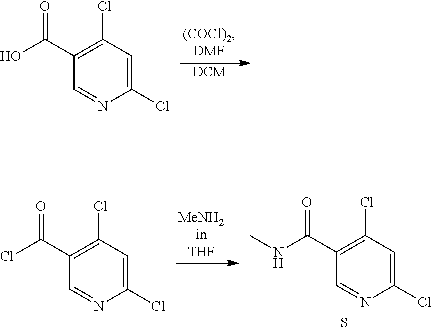

Step 1—4,6-dichloronicotinoyl chloride. To a stirred solution of 4,6-dichloropyridine-3-carboxylic acid (100 g, 521 mmol) and DMF (3.81 g, 52.0 mmol, 4.0 mL) in DCM (1 L) was added (COCl)₂ (198 g, 1.56 mol, 137 mL) dropwise at 0° C. under nitrogen atmosphere. The resulting mixture was stirred for 12 h at 15° C. under nitrogen atmosphere. On completion, the reaction solution was used directly in the next step.

Step 2—4,6-dichloro-N-methylnicotinamide. To a solution of MeNH2 (2 M solution in THF, 524 mL) was added 4,6-dichloropyridine-3-carbonyl chloride (100 g, 475.18 mmol) dropwise over 15 min at 0° C. The resulting mixture was stirred for 2 h at 15° C. On completion, the residue was poured into water (400 mL) and stirred for 20 min. The aqueous phase was extracted with ethyl acetate (150 mL×3). The combined organic phase was washed with brine (100 mL), dried with anhydrous Na2SO₄, filtered and concentrated in vacuo. The residue was purified by column chromatography (SiO2, PE:EA=5:1 to 1:1) to give the title compound (60 g, 62% yield) as a white solid. 1H NMR (400 MHz, CDCl3) δ=8.47 (s, 1H), 6.83 (brs, 1H), 2.95 (s, 3H).

### Tert-butyl 4-(4,4,5,5-tetramethyl-1,3,2-dioxaborolan-2-yl)indoline-1-carboxylate (Intermediate MA)

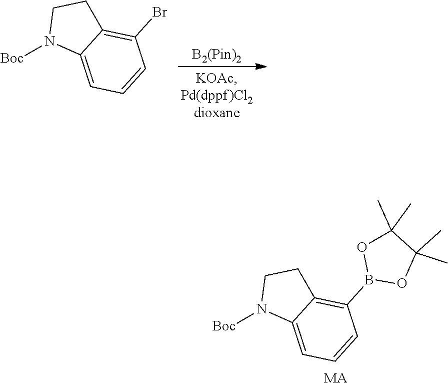

To a stirred mixture of tert-butyl 4-bromoindoline-i-carboxylate (50.00 g, 167.7 mmol, CAS #885272-46-8) and 4,4,4′,4′,5,5,5′,5′-octamethyl-2,2′-bi(1,3,2-dioxaborolane) (63.87 g, 251.5 mmol) in dioxane (600 mL) were added KOAc (49.37 g, 503.1 mmol) and Pd(dppf)Cl2CH2Cl2 (13.66 g, 16.77 mmol) at rt under nitrogen atmosphere. The resulting mixture was stirred for 2 h at 90° C. under nitrogen atmosphere. On completion, the mixture was cooled to rt and concentrated under reduced pressure. The residue was purified by silica gel column chromatography, eluted with PE/EA (10:1), to afford the title compound (56.00 g, 96% yield) as a yellow solid. LC/MS (ESI, m/z): [(M+1)]⁺=346.2.

### 5-Fluoro-6-(indolin-4-yl)nicotinaldehyde (Intermediate MB)

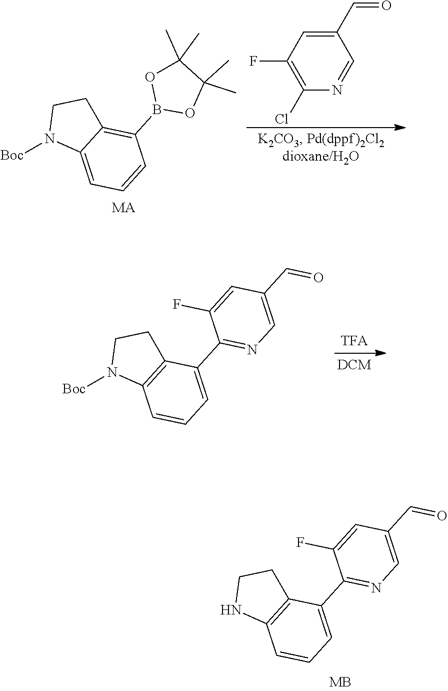

Step 1—Tert-butyl 4-(3-fluoro-5-formylpyridin-2-yl)indoline-1-carboxylate. To a stirred solution of tert-butyl 4-(4,4,5,5-tetramethyl-1,3,2-dioxaborolan-2-yl)indoline-1-carboxylate (25.00 g, 72.46 mmol, Intermediate MA) and 6-chloro-5-fluoronicotinaldehyde (12.67 g, 79.71 mmol, CAS #950691-52-8) in dioxane (380 mL) and H₂O (76 mL) were added K₂CO₃ (30.00 g, 217.4 mmol) and Pd(dppf)C₁₋₂·CH₂Cl₂ (5.91 g, 7.25 mmol) at rt. The resulting mixture was then stirred for 2 h at 90° C. under nitrogen atmosphere. On completion, the mixture was cooled to rt and concentrated under reduced pressure. The residue was purified by silica gel column chromatography, eluted with PE/EA (5:1), to afford the title compound (17.00 g, 68% yield) as a light yellow oil. LC/MS (ESI, m/z): [(M+1)]⁺=343.1.

Step 2—5-fluoro-6-(indolin-4-yl)nicotinaldehyde. To a stirred mixture of tert-butyl 4-(3-fluoro-5-formylpyridin-2-yl)indoline-1-carboxylate (17.00 g, 49.71 mmol) in DCM (160 mL) was added TFA (40 mL) dropwise at rt. The resulting mixture was stirred for 1 h at rt. On completion, the mixture was basified to pH 8 with saturated Na₂CO₃ (aq.). The resulting mixture was diluted with water (200 mL) and extracted with EtOAc (3×150 mL). The combined organic layers were washed with brine (1×500 mL), dried over anhydrous Na₂SO₄, filtered, and the filtrate was concentrated under reduced pressure to afford the title compound (12.00 g) as a yellow solid, LC/MS (ESI, m/z): [(M+1)]⁺=242.1.

### 6-(4-(3-Fluoro-5-formylpyridin-2-yl)indolin-1-yl)-N-((1R,2S)-2-fluorocyclopropyl)-8-(methylamino)imidazo[1,2-b]pyridazine-3-carboxamide 2,2,2-trifluoroacetate (Intermediate MC)

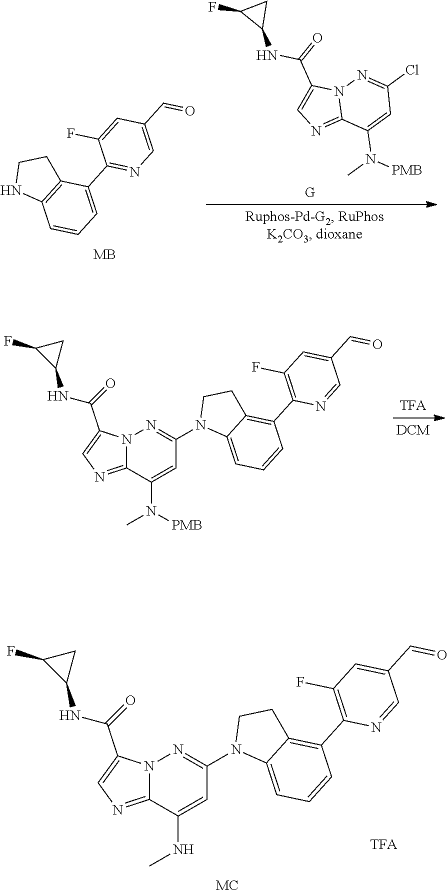

Step 1—6-(4-(3-Fluoro-5-formylpyridin-2-yl)indolin-1-yl)-N-((1R,2S)-2-fluorocyclopropyl)-8-((4-methoxybenzyl)(methyl)amino)imidazo[1,2-b]pyridazine-3-carboxamide. To a stirred mixture of 6-chloro-N-((1R,2S)-2-fluorocyclopropyl)-8-((4-methoxybenzyl)(methyl)amino)imidazo[1,2-b]pyridazine-3-carboxamide (16.65 g, 41.32 mmol, Intermediate G), 5-fluoro-6-(indolin-4-yl)nicotinaldehyde (12.00 g, 49.59 mmol, Intermediate MB) and K₂CO₃ (17.10 g, 123.94 mmol) in dioxane (200 mL) were added RuPhos (1.93 g, 4.13 mmol) and RuPhos-PdCl-2nd G (3.21 g, 4.13 mmol) at rt under nitrogen atmosphere. The resulting mixture was stirred for 2 h at 100° C. under nitrogen atmosphere. On completion, the mixture was cooled to rt and concentrated under reduced pressure. The residue was purified by silica gel column chromatography, eluted with CH₂Cl₂/EA (1:1), to afford the title compound (18.00 g, 71% yield) as a yellow solid. LC/MS (ESI, m/z): [(M+1)]⁺=610.2.

Step 2—6-(4-(3-Fluoro-5-formylpyridin-2-yl)indolin-1-yl)-N-((1R,2S)-2-fluorocyclopropyl)-8-(methylamino)imidazo[1,2-b]pyridazine-3-carboxamide 2,2,2-trifluoroacetate. To a stirred mixture of 6-(4-(3-fluoro-5-formylpyridin-2-yl)indolin-1-yl)-N-((1R,2S)-2-fluorocyclopropyl)-8-((4-methoxybenzyl)(methyl)amino)imidazo[1,2-b]pyridazine-3-carboxamide (18.00 g, 29.56 mmol) in DCM (150 mL) was added TFA (50 mL) dropwise at rt and the mixture was stirred for 1 h at rt. On completion, the mixture was concentrated under reduced pressure. The residue was washed with Et₂O (3×100 mL) to afford the title compound (17 g, TFA salt) as a yellow solid. LC/MS (ESI, m/z): [(M+1)]⁺=490.1; ¹H NMR (400 MHz, DMSO-d₆) δ 10.19 (d, J=2.0 Hz, 1H), 9.08 (t, J=1.6 Hz, 1H), 8.85 (d, J=4.8 Hz, 1H), 8.28 (dd, J=10.0, 1.6 Hz, 1H), 7.97 (s, 1H), 7.86 (d, J=8.0 Hz, 1H), 7.77-7.68 (m, 1H), 7.34 (t, J=8.0 Hz, 1H), 7.25-7.16 (m, 1H), 6.02 (s, 1H), 5.00-4.75 (m, 1H), 4.17 (t, J=8.4 Hz, 2H), 3.24 (t, J=8.4 Hz, 2H), 3.10-3.00 (m, 1H), 2.97 (d, J=4.8 Hz, 3H), 1.30-1.16 (m, 1H), 1.07-0.89 (m, 1H).

### 6-(4-(5-((5,5-Difluoro-2,7-diazaspiro[3.5]nonan-2-yl)methyl)-3-fluoropyridin-2-yl)indolin-1-yl)-N-((1R,2S)-2-fluorocyclopropyl)-8-(methylamino)imidazo[1,2-b]pyridazine-3-carboxamide trifluoroacetate (Intermediate MD)

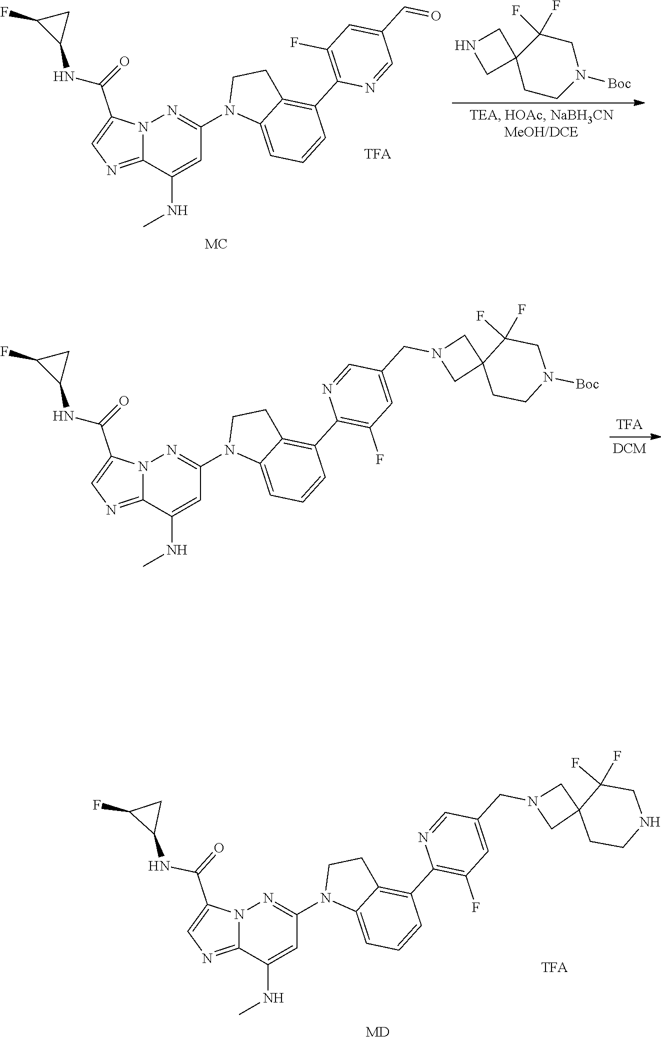

Step 1—Tert-butyl 5,5-difluoro-2-((5-fluoro-6-(1-(3-(((1R,2S)-2-fluorocyclopropyl)carbamoyl)-8-(methylamino)imidazo[1,2-b]pyridazin-6-yl)indolin-4-yl)pyridin-3-yl)methyl)-2,7-diazaspiro[3.5]nonane-7-carboxylate. To a stirred mixture of 6-(4-(3-fluoro-5-formylpyridin-2-yl)indolin-1-yl)-N-((1R,2S)-2-fluorocyclopropyl)-8-(methylamino)imidazo[1,2-b]pyridazine-3-carboxamide trifluoroacetate (17.00 g, 34.76 mmol, Intermediate MC) and tert-butyl 5,5-difluoro-2,7-diazaspiro[3.5]nonane-7-carboxylate (9.11 g, 34.76 mmol. CAS #1228631-69-3) in MeOH (100 mL) and DCE (100 mL) were added Et₃N (7.02 g, 69.5 mmol) and HOAc (20.86 g, 347.7 mmol) dropwise at rt. The resulting mixture was stirred for 4 h at 50° C. To the above mixture was added NaBH₃CN (10.95 g, 173.8 mmol) in portions at rt. The resulting mixture was stirred for additional 1 h at rt. On completion, the mixture was concentrated under reduced pressure. The residue was purified by reversed-phase flash chromatography (Column: Spherical C18, 2040 μm, 330 g; Mobile Phase A: Water(plus 10 mM NH4HCO₃), Mobile Phase B: ACN; Flow rate: 80 mL/min; Gradient (B %): 5%-40%, 4 min; 40%˜60%, 20 min; 60%˜95%; 2 min; 95%, 5 min; Detector: 254 nm; the fractions containing desired product were collected at 58% B) and concentrated under reduced pressure to afford the title compound (15.00 g, 59% yield) as a white solid. LC/MS (ESI, m/z): [(M+1)]⁺=736.3.

Step 2—6-(4-(5-((5,5-Difluoro-2,7-diazaspiro[3.5]nonan-2-yl)methyl)-3-fluoropyridin-2-yl)indolin-1-yl)-N-((1R,2S)-2-fluorocyclopropyl)-8-(methylamino)imidazo[1,2-b]pyridazine-3-carboxamide trifluoroacetate. To a stirred mixture of tert-butyl 5,5-difluoro-2-((5-fluoro-6-(1-(3-(((1R,2S)-2-fluorocyclopropyl)carbamoyl)-8-(methylamino)imidazo[1,2-b]pyridazin-6-yl)indolin-4-yl)pvridin-3-yl)methyl)-2,7-diazaspiro[3.5]nonane-7-carboxylate (15.00 g, 20.41 mmol) in DCM (150 mL) was added TFA (50 mL) dropwise at rt and the mixture was stirred for 1 h at rt. The resulting mixture was concentrated under reduced pressure. The residue was washed with Et₂O (3×100 mL) to afford the title compound (15 g, TFA salt) as a light yellow solid. LC/MS (ESI, m/z): [(M+1)]⁺=635.3.

### 4-chloro-3-(2,4-dioxotetrahydropyrimidin-1(2H)-yl)-5-methylbenzoic acid (Intermediate ME)

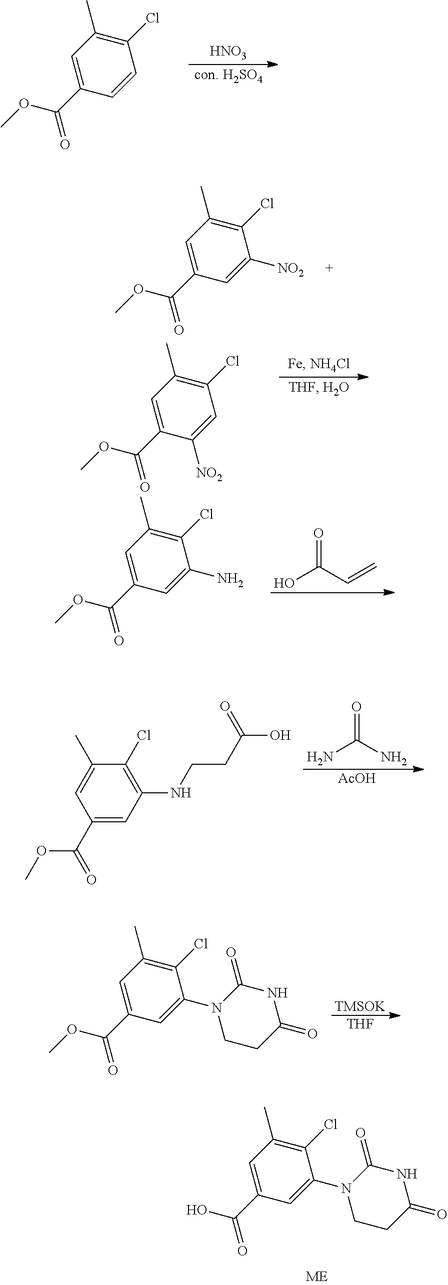

Step 1—Methyl 4-chloro-3-methyl-5-nitrobenzoate and methyl 4-chloro-5-methyl-2-nitrobenzoate. To a stirred solution of methyl 4-chloro-3-methylbenzoate (100.00 g, 543.48 mmol) in H₂SO₄ (150 mL) was added nitric acid (100 mL) at 0° C. under nitrogen atmosphere. The resulting mixture was stirred for 1 h at rt under nitrogen atmosphere. On completion, the reaction was quenched with water/ice at 0° C., and extracted with EtOAc (3×500 mL). The combined organic layers were washed with brine (1×1000 mL), dried over anhydrous Na₂SO₄, filtered, and the filtrate was concentrated under reduced pressure. The residue was purified by silica gel column chromatography, eluted with PE/EA (10:1), to afford the mixture of methyl 4-chloro-3-methyl-5-nitrobenzoate and methyl 4-chloro-5-methyl-2-nitrobenzoate (˜1/1) (110 g, 88% yield) as an orange solid.

Step 2—Methyl 3-amino-4-chloro-5-methylbenzoate. To a stirred solution of methyl 4-chloro-3-methyl-5-nitrobenzoate (110.00 g, 480.35 mmol) in THF (2 L) and H₂O (1 L) were added NH₄C₁ (254.58 g, 4.80 mol) and Fe (269.00 g, 4.80 mol) at rt under nitrogen atmosphere. The resulting mixture was stirred for 16 h at 60° C. under nitrogen atmosphere. On completion, the mixture was filtered, and the filter cake was washed with DCM (5×500 mL). The filtrate was concentrated under reduced pressure and the mixture was extracted with EtOAc (3×500 mL). The combined organic layers were washed with brine (1×1000 mL), dried over anhydrous Na₂SO₄, filtered, and the filtrate was concentrated under reduced pressure. The residue was purified by silica gel column chromatography, eluted with PE/EA (4:1), to afford the title compound (40.00 g, 41% yield) as a light yellow solid. LC/MS (ESI, m/z): [(M+1)]⁺=200.0; ¹H NMR (300 MHz, DMSO-d₆) δ 7.30 (d, J=2.0 Hz, 111), 7.10 (d, J=2.0 Hz, 1H), 5.58 (s, 2H), 3.81 (s, 3H), 2.30 (s, 3H).

Step 3—3-((2-Chloro-5-(methoxycarbonyl)-3-methylphenyl)amino)propanoic acid. To a stirred solution of methyl 3-amino-4-chloro-5-methylbenzoate (40.00 g, 201.0 mmol) was added acrylic acid (28.94 g, 402.0 mmol) at rt under nitrogen atmosphere. The resulting mixture was stirred for 4 h at 100° C. under nitrogen atmosphere. On completion, the mixture was cooled to rt and diluted with water (500 mL). The resulting mixture was extracted with EtOAc (3×500 mL). The combined organic layers were washed with brine (1×1000 mL), dried over anhydrous Na₂SO₄, filtered, and the filtrate was concentrated under reduced pressure to give the title compound (60 g) as an orange solid. LC/MS (ESI, m/z): [(M+1)]⁺=272.1.

Step 4—Methyl 4-chloro-3-(2,4-dioxotetrahydropyrimidin-1(2H)-yl)-5-methylbenzoate. To a stirred solution of 3-((2-chloro-5-(methoxycarbonyl)-3-methylphenyl)amino)propanoic acid (60.00 g, 221.4 mmol) in HOAc (450 mL) was added urea (92.99 g, 1.55 mol) at rt under nitrogen atmosphere. The resulting mixture was stirred for 16 h at 110° C. under nitrogen atmosphere. On completion, the mixture was cooled to rt and the solution was poured into ice water where solid particles precipitated. The precipitated solids were collected by filtration and washed with water (3×100 mL) and acetonitrile (2×50 mL). Then the solid was dried under reduced pressure to give the title compound (30.00 g, 51% yield) as an off white solid. LC/MS (ESI, m/z): [(M+1)]⁺=297.2; ¹H NMR (400 MHz, DMSO-d₆) δ 7.98-7.89 (m, 2H), 3.87 (s, 3H), 3.79-3.71 (m, 1H), 3.66-3.57 (m, 1H), 2.84-2.70 (m, 2H), 2.45 (s, 3H).

Step 5—4-Chloro-3-(2,4-dioxotetrahydropyrimidin-1(2H)-yl)-5-methylbenzoic acid. To a stirred solution of methyl 4-chloro-3-(2,4-dioxotetrahydropyrimidin-1(2H)-yl)-5-methylbenzoate (30.00 g, 101.4 mmol) in THF (750 mL) was added potassium trimethylsilanolate (51.89 g, 405.4 mmol) at rt under nitrogen atmosphere. The resulting mixture was stirred for 1 h at rt under nitrogen atmosphere. On completion, the mixture was poured 2 N HCl slowly. The precipitated solids were collected by filtration and washed with water (3×50 mL). Then the solid was dried under reduced pressure to give the title compound (20 g, 70% yield) as an off-white solid. LC/MS (ESI, m/z): [(M+1)]⁺=283.0. ¹H NMR (300 MHz, DMSO-d₆) δ 10.38 (s, 1H), 7.77 (d, J=2.0 Hz, 1H), 7.67 (d, J=2.0 Hz, 1H), 3.76-3.51 (m, 2H), 2.82-2.65 (m, 2H), 2.36 (s, 3H).

### 6-Chloro-N-[(1R,2R)-2-methoxycyclobutyl]-8-{[(4-methoxyphenyl)methyl](methyl)amino}imidazo[1,2-b]pyridazine-3-carboxamide (1.6 g, 3.722 mmol) (Intermediate MF)

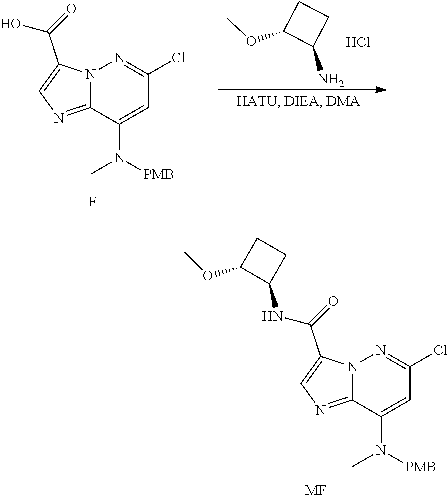

To a stirred solution of 6-chloro-8-{[(4-methoxyphenyl)methyl](methyl)amino}imidazo[1,2-b]pyridazine-3-carboxylic acid (50 g, 140 mmol, Intermediate F) and (1R,2R)-2-methoxycyclobutan-1-amine hydrochloride (21.83 g, 158.6 mmol) in DMA (200 mL) were added DIEA (55.91 g, 432.6 mmol) and HATU (82.24 g, 216.3 mmol) at 0° C. under nitrogen atmosphere. The resulting mixture was stirred for 1 h at rt under nitrogen atmosphere. On completion, the mixture was concentrated under reduced pressure. The residue was purified by reverse flash chromatography (Column: WelFlash™ C18-I, 20-40 μm, 120 g; Eluent A: Water (plus 10 mmol/L FA); Eluent B: ACN; Gradient: 30%-50% B in 30 min; Flow rate: 60 mL/min; Detector: 220/254 nm; desired fractions were collected at 36% B) and concentrated under reduced pressure to afford the title compound (60 g, 97% yield) as a white solid. LC/MS (ESI, m/z): [(M+H)]+=430.2.

### 6-[4-(3-Fluoro-5-formylpyridin-2-yl)-2,3-dihydroindol-1-yl]-N-[(1R,2R)-2-methoxycyclobutyl]-8-(methylamino)imidazo[1,2-b]pyridazine-3-carboxamide trifluoroacetate (Intermediate MG)

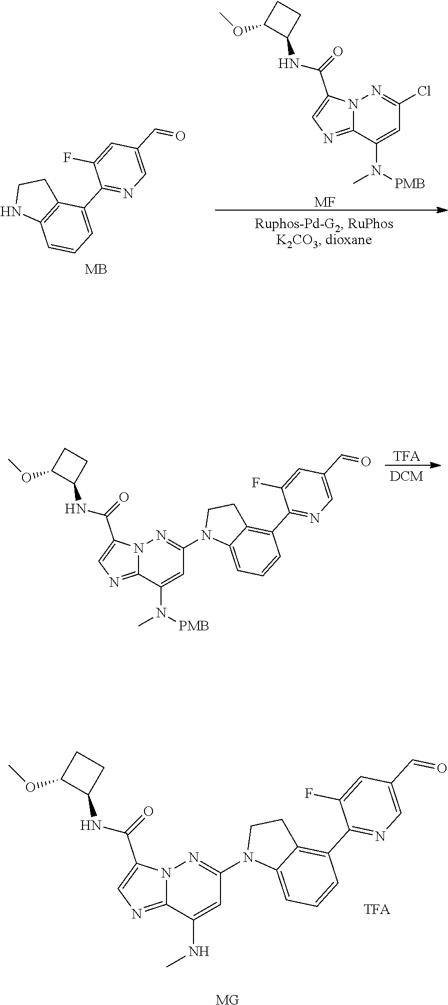

Step 1—6-[4-(3-Fluoro-5-formylpyridin-2-yl)-2,3-dihydroindol-1-yl]-N-[(1R,2R)-2-methoxycyclobutyl]-8-{[(4-methoxyphenyl)methyl](methyl)amino}imidazo[1,2-b]pyridazine-3-carboxamide. To a stirred mixture of 6-(2,3-dihydro-1H-indol-4-yl)-5-fluoropyridine-3-carbaldehyde (1.26 g, 5.21 mmol, Intermediate MB), 6-chloro-N-[(1R,2R)-2-methoxycyclobutyl]-8-{[(4-methoxyphenyl)methyl](methyl)amino}imidazo[1,2-b]pyridazine-3-carboxamide (1.6 g, 3.7 mmol, Intermediate MF) and K₂CO₃ (1.54 g, 11.2 mmol) in dioxane (16 mL) were added RuPhos (0.17 g, 0.37 mmol) and RuPhos-PdCl-2nd G (0.29 g, 0.37 mmol) at rt under nitrogen atmosphere. The resulting mixture was stirred for 1 h at 110° C. under nitrogen atmosphere. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH4HCO₃); Eluent B: ACN; Gradient: 60%-80% B in 25 min; Flow rate: 80 mL/min; Detector: 220/254 nm; desired fractions were collected at 70% B) and concentrated under reduced pressure to afford the title compound (2.3 g, 97% yield) as a yellow solid. LC/MS (ESI, m/z): [(M+H)]⁺=636.2.

Step 2—6-[4-(3-Fluoro-5-formylpyridin-2-yl)-2,3-dihydroindol-1-yl]-N-[(1R,2R)-2-methoxycyclobutyl]-8-(methylamino)imidazo[1,2-b]pyridazine-3-carboxamide trifluoroacetate. To a stirred solution of 6-[4-(3-fluoro-5-formylpyridin-2-yl)-2,3-dihydroindol-1-yl]-N-[(1R,2R)-2-methoxycyclobutyl]-8-{[(4-methoxyphenyl)methyl](methyl)amino}imidazo[1,2-b]pyridazine-3-carboxamide (2.3 g, 3.6 mmol) in DCM (18 mL) was added TFA (6 mL) at rt and the mixture was stirred for 1 h at rt. On completion, the mixture was concentrated under reduced pressure. The residue was purified by trituration with Et₂O (100 mL) to afford the title compound (2.2 g, 99% yield) as a yellow solid. LC/MS (ESI, m/z): [(M+H)]⁺=516.1.

### 6-{4-[5-({5,5-Difluoro-2,7-diazaspiro[3.5]nonan-2-yl}methyl)-3-fluoropyridin-2-yl]-2,3-dihydroindol-1-yl}-N-[(1R,2R)-2-methoxycyclobutyl]-8-(methylamino)imidazo[1,2-b]pyridazine-3-carboxamide trifluoroacetate (Intermediate MH)

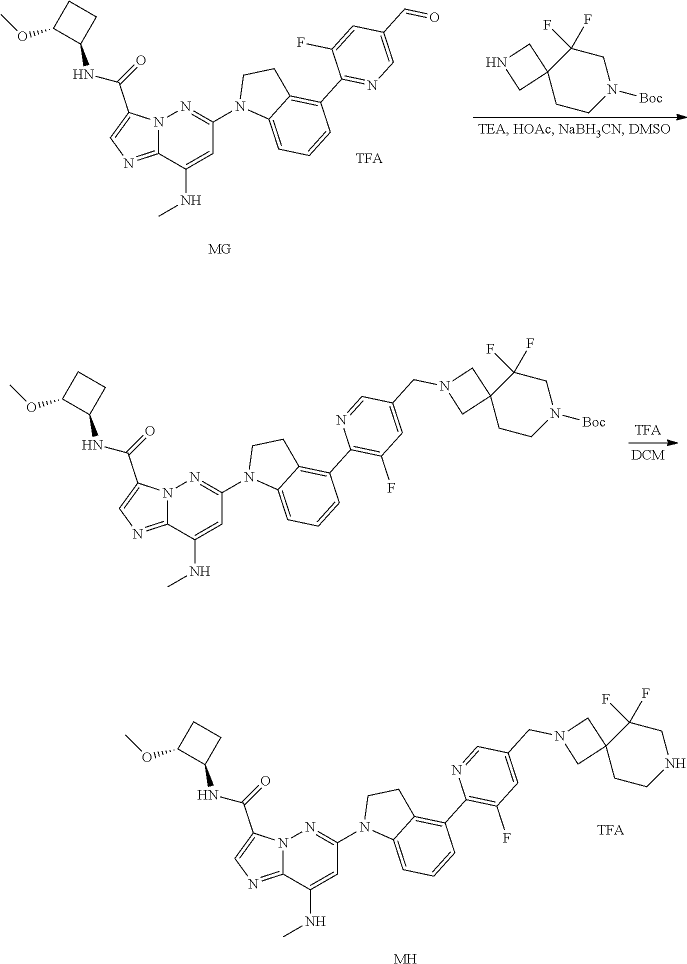

Step 1—Tert-butyl 5,5-difluoro-2-({5-fluoro-6-[1-(3-{[(1R,2R)-2-methoxycyclobutyl]carbamoyl}-8-(methylamino)imidazo[1,2-b]pyridazin-6-yl)-2,3-dihydroindol-4-yl]pyridin-3-yl}methyl)-2,7-diazaspiro[3.5]nonane-7-carboxylate. To a stirred mixture of 6-[4-(3-fluoro-5-formylpyridin-2-yl)-2,3-dihydroindol-1-yl]-N-[(1R,2R)-2-methoxycyclobutyl]-8-(methylamino)imidazo[1,2-b]pyridazine-3-carboxamide trifluoroacetate (1 g, 2 mmol, Intermediate MG) in DMSO (15 mL) was added TEA (0.66 mL, 4.76 mmol) at rt under nitrogen atmosphere. To the above mixture were added tert-butyl 5,5-difluoro-2,7-diazaspiro[3.5]nonane-7-carboxylate (0.42 g, 1.6 mmol, CAS #1228631-69-3) and HOAc (0.46 mL, 7.940 mmol) at rt. The resulting mixture was stirred for an additional 30 min at 50° C. Next, NaBH₃CN (0.20 g, 3.18 mmol) was added to the mixture at rt. The resulting mixture was stirred for an additional 1 h at 50° C. On completion, the mixture was cooled to rt and concentrated under vacuum. The residue was purified by reverse phase flash chromatography (Column: WelFlash™ C18-I, 20-40 μm, 330 g; Eluent A: Water (plus 10 mmol/L NH4HCO₃); Eluent B: ACN; Gradient: 60%-80% B in 25 min; Flow rate: 80 mL/min; Detector: 220/254 nm; desired fractions were collected at 67% B) and concentrated under reduced pressure to afford the title compound (900 mg, 74% yield) as a white solid. LC/MS (ESI, m/z): [(M+H)]⁺=762.4.

Step 2—6-{4-[5-({5,5-Difluoro-2,7-diazaspiro[3.5]nonan-2-yl}methyl)-3-fluoropyridin-2-yl]-2,3-dihydroindol-1-yl}-N-[(1R,2R)-2-methoxycyclobutyl]-8-(methylamino)imidazo[1,2-b]pyridazine-3-carboxamide trifluoroacetate. To a stirred solution of tert-butyl 5,5-difluoro-2-({5-fluoro-6-[1-(3-{[(1R,2R)-2-methoxycyclobutyl]carbamoyl}-8-(methylamino)imidazo[1,2-b]pyridazin-6-yl)-2,3-dihydroindol-4-yl]pyridin-3-yl}methyl)-2,7-diazaspiro[3.5]nonane-7-carboxylate (900 mg, 1.181 mmol) in DCM (10 mL) was added TFA (5 mL) at rt under nitrogen atmosphere and the mixture was stirred for 1 h at rt. On completion, the mixture was concentrated under reduced pressure. The residue was purified by trituration with Et₂O (100 mL) to give the title compound (900 mg, 98% yield) as a white solid. LC/MS (ESI, m/z): [(M+H)]⁺=662.3.

### 6-Fluoroindoline-4-carbaldehyde (Intermediate MI)

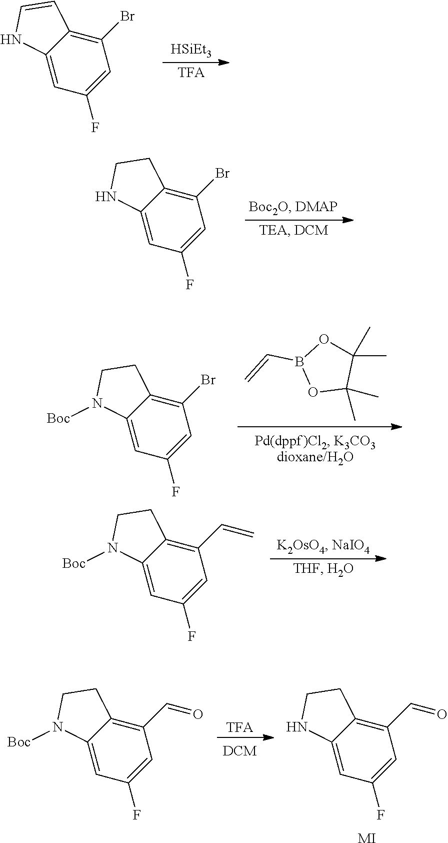

Step 1—4-Bromo-6-fluoroindoline. To a stirred mixture of 4-bromo-6-fluoro-1H-indole (25.00 g, 116.8 mmol, CAS #885520-70-7) in TFA (120 mL) was added triethylsilane (40.74 g, 350.4 mmol) at 0° C. under nitrogen atmosphere. The mixture was then allowed to warm to rt and stirred for 1 h at rt under nitrogen atmosphere. On completion, the mixture was basified to pH 7 with saturated NaOH. The resulting mixture was extracted with EtOAc (3×500 mL). The combined organic layers were washed with water (3×500 mL), dried over anhydrous Na₂SO₄, filtered, and the filtrate was concentrated under reduced pressure. The residue was purified by silica gel column chromatography, eluted with petroleum ether/EtOAc (3:1), to afford the title compound (25.00 g, 99% yield) as a yellow oil. LC/MS (ESI, m/z): [(M+1)]⁻=216.1, 218.1.

Step 2—Tert-butyl 4-bromo-6-fluoroindoline-1-carboxylate. To a stirred solution of 4-bromo-6-fluoroindoline (16.00 g, 74.06 mmol) in DCM (200 mL) were added TEA (14.99 g, 148.1 mmol) and Boc₂O (19.40 g, 88.87 mmol) at rt under air atmosphere. The resulting mixture was stirred 16 h at rt under nitrogen atmosphere. On completion, the mixture was concentrated under vacuum. The residue was purified by silica gel column chromatography, eluted with petroleum ether/EtOAc (5:1), to afford the title compound (12 g, 51% yield) as a yellow solid. LC/MS (ESI, m/z): [(M+1)]⁺=316.2, 318.2.

Step 3—Tert-butyl 6-fluoro-4-vinylindoline-1-carboxylate. To a stirred solution of tert-butyl 4-bromo-6-fluoroindoline-1-carboxylate (10.00 g, 31.63 mmol) and 4,4,5,5-tetramethyl-2-vinyl-1,3,2-dioxaborolane (5.41 g, 35.1 mmol) in dioxane (100 mL) and H₂O (20 mL) were added K₂CO₃ (12.98 g, 93.94 mmol) and Pd(dppf)C₁₋₂·CH₂Cl₂ (2.58 g, 3.16 mmol) at rt. The resulting mixture was stirred for 1 h at 90° C. under nitrogen atmosphere. On completion, the mixture was cooled to rt and concentrated under reduced pressure. The residue was purified by silica gel column chromatography, eluted with petroleum ether/EtOAc (4:1), to afford the title compound (4.61 g, 550% yield) as a white solid. LC/MS (ESI, m/z): [(M+1)]⁺=263.3.

Step 4—Tert-butyl 6-fluoro-4-formylindoline-1-carboxylate. To a stirred solution of tert-butyl 6-fluoro-4-vinylindoline-1-carboxylate (7.00 g, 26.6 mmol) and NaIO₄ (28.43 g, 132.9 mmol) in THF (100 mL) and H₂O (50 mL) was added K₂OsO₄·2H₂O (0.49 g, 1.33 mmol) at 0° C. The resulting mixture was stirred for 1 h at rt. On completion, the reaction was quenched by the addition of water (300 mL) at rt and extracted with EtOAc (3×200 mL). The combined organic layers were washed with brine (3×500 mL), dried over anhydrous Na₂SO₄, filtered, and the filtrate was concentrated under reduced pressure. The residue was purified by silica gel column chromatography, eluted with petroleum ether/EtOAc (3:1), to afford the title compound (3.31 g, 47% yield) as a white solid. LC/MS (ESI, m/z): [(M+1)]⁺=266.3.

Step 5—6-Fluoroindoline-4-carbaldehyde. To a stirred solution of tert-butyl 6-fluoro-4-formylindoline-1-carboxylate (3.30 g, 12.4 mmol) in DCM (60 mL) was added TFA (15 mL) at rt. The resulting mixture was stirred for 1 h at rt. On completion, the mixture was basified to pH 8 with saturated Na₂CO₃ (aq.) and extracted with CH₂C₁₋₂ (3×200 mL). The combined organic layers were washed with brine (1×300 mL), dried over anhydrous Na₂SO₄, filtered, and the filtrate was concentrated under reduced pressure to give the title compound (2.02 g, 98% yield) as a brown solid. LC/MS (ESI, m/z): [(M-1)]⁻=164.1.

### 6-(6-Fluoro-4-formylindolin-1-yl)-N-((1R,2R)-2-methoxycyclobutyl)-8-(methylamino)imidazo[1,2-b]pyridazine-3-carboxamide 2,2,2-trifluoroacetate (Intermediate MJ)

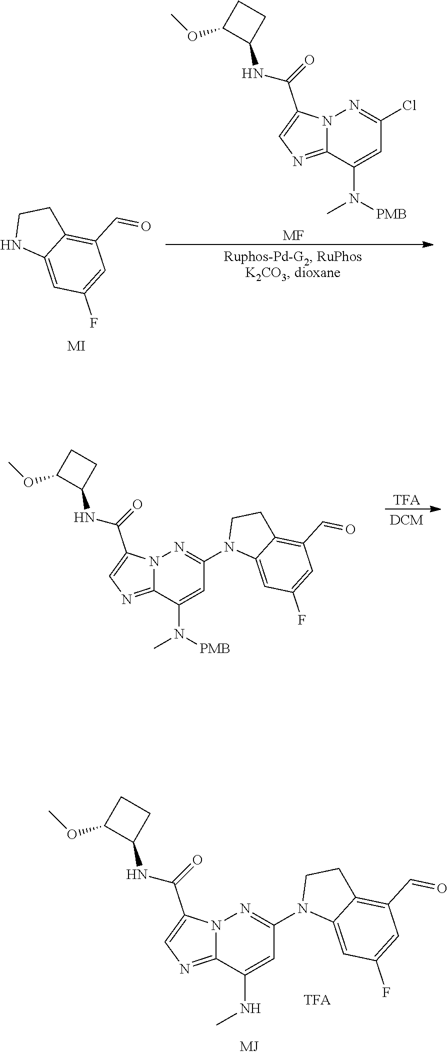

Step 1—6-(6-fluoro-4-formylindolin-1-yl)-8-((4-methoxybenzyl)(methyl)amino)-N-((1R,2R)-2-methoxycyclobutyl)imidazo[1,2-b]pyridazine-3-carboxamide. To a stirred solution of 6-fluoroindoline-4-carbaldehyde (2.00 g, 12.1 mmol, Intermediate MI), K₂CO₃ (3.35 g, 24.2 mmol) and 6-chloro-8-((4-methoxybenzyl)(methyl)amino)-N-((1R,2R)-2-methoxycyclobutyl)imidazo[1,2-b]pyridazine-3-carboxamide (3.47 g, 8.08 mmol, Intermediate MF) in 1,4-dioxane (30 mL) were added RuPhos (0.75 g, 1.62 mmol) and RuPhos-PdCl-2nd G (0.63 g, 0.81 mmol) at rt under nitrogen atmosphere. The resulting mixture was stirred for 1 h at 100° C. under nitrogen atmosphere. On completion, the mixture was cooled to rt and purified by reversed-phase flash chromatography (column, C18 silica gel; mobile phase, MeCN in Water (10 mmol/L NH4HCO₃), 50% to 95% gradient in 30 min; detector, UV 254 nm) to afford the title compound (2.3 g, 50% yield) as a light yellow solid. LC/MS (ESI, m/z): [(M+1)]⁺=559.4.

Step 2—6-(6-Fluoro-4-formylindolin-1-yl)-N-((1R,2R)-2-methoxycyclobutyl)-8-(methylamino)imidazo[1,2-b]pyridazine-3-carboxamide 2,2,2-trifluoroacetate. To a stirred solution of 6-(6-fluoro-4-formylindolin-1-yl)-8-((4-methoxybenzyl)(methyl)amino)-N-((1R,2R)-2-methoxycyclobutyl)imidazo[1,2-b]pyridazine-3-carboxamide (2.30 g, 4.12 mmol) in DCM (30 mL) was added TFA (10 mL) at rt and the mixture was stirred for 1 h at rt. On completion, the mixture was concentrated under reduced pressure. The residue was purified by trituration with Et₂O (40 mL) to afford the title compound (1.80 g, 99% yield) as a brown solid. LC/MS (ESI, m/z): [(M+1)]⁺=439.3.

### Tert-butyl 9-(3,3-difluoropiperidin-4-yl)-3,9-diazaspiro[5.5]undecane-3-carboxylate (Intermediate MK)

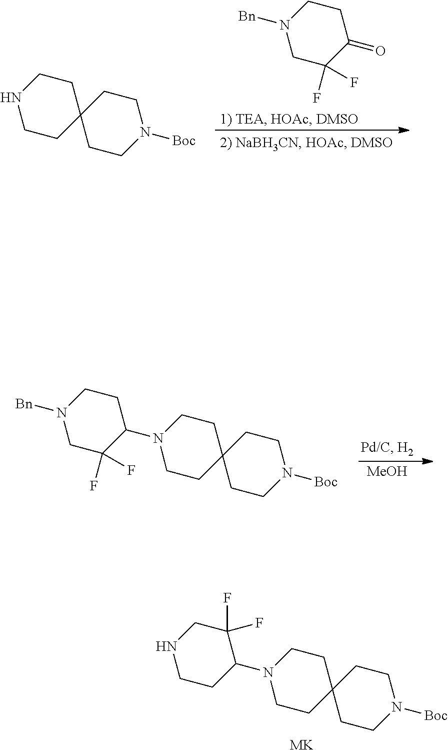

Step 1—Tert-butyl 9-(1-benzyl-3,3-difluoropiperidin-4-yl)-3,9-diazaspiro[5.5]undecane-3-carboxylate. To a stirred mixture of tert-butyl 3,9-diazaspiro[5.5]undecane-3-carboxylate (200 g, 800 mmol) in DMSO (1000 mL) was added TEA (327.87 mL, 2358.7 mmol) at rt under nitrogen atmosphere and the mixture was stirred for 5 min at rt under nitrogen atmosphere. To the above mixture was added 1-benzyl-3,3-difluoropiperidin-4-one (265.64 g, 1179.4 mmol, CAS #1039741-54-2) and HOAc (225.26 mL, 3931.2 mmol) at rt. The resulting mixture was stirred for additional 2 h at 100° C. After the imine was generated completely, the reaction mixture was allowed to cool down to rt. Then MeOH (1000 mL) and HOAc (100 mL) was added to the above mixture at rt under nitrogen atmosphere, followed by addition of NaBH₃CN (54.45 g, 866.6 mmol) in portions at rt. The resulting mixture was stirred for 2 h at 50° C. under nitrogen atmosphere. On completion, the reaction mixture cooled to rt and concentrated under reduced pressure. The mixture was diluted with water (1000 mL) and was extracted with EtOAc (3×3000 mL). The combined organic layers were dried over anhydrous Na₂SO₄, filtered, and the filtrate was concentrated under reduced pressure. The residue was purified by silica gel column chromatography, eluted with DCM/EA (10:1), to afford the crude product (170 g) as a white solid. The crude was further purified by reverse flash chromatography (column, C18 silica gel; mobile phase A: water (10 mmol/L NH4HCO₃), mobile phase B: ACN, 60% to 90% gradient in 30 min; detector, UV 254 nm; the fractions were collected at 73%) and concentrated under reduced pressure to afford the title compound (110 g, 30% yield for two steps) as a white solid. ¹H NMR (300 MHz, DMSO-d₆) δ 7.37-7.23 (m, 5H), 3.61-3.48 (m, 2H), 3.29-3.22 (m, 4H), 3.00-2.82 (m, 2H), 2.81-2.70 (m, 1H), 2.70-2.61 (m, 4H), 2.31-2.02 (m, 2H), 1.86-1.67 (m, 2H), 1.44-1.35 (m, 13H), 1.35-1.27 (m, 4H); LC/MS (ESI, m/z): [(M+H)]⁺=464.3.

Step 2—Tert-butyl 9-(3,3-difluoropiperidin-4-yl)-3,9-diazaspiro[5.5]undecane-3-carboxylate. To a stirred mixture of tert-butyl 9-(1-benzyl-3,3-difluoropiperidin-4-yl)-3,9-diazaspiro[5.5]undecane-3-carboxylate (4.6 g, 9.9 mmol) in MeOH (50 mL) was added Pd/C (1.2 g) in portions at rt under nitrogen atmosphere and the mixture was stirred for 2 h at rt under hydrogen atmosphere. After completion of the reaction, the resulting mixture was filtered, the filter cake was washed with EtOAc (3×100 mL). The filtrate was concentrated under reduced pressure to afford the title compound (3.2 g, 86% yield) as a white solid. LC/MS (ESI, m/z): [(M+H)]⁺=374.3.

### 6-(4-((3,3-Difluoro-4-(3,9-diazaspiro[5.5]undecan-3-yl)piperidin-1-yl)methyl)-6-fluoroindolin-1-yl)-N-((1R,2R)-2-methoxycyclobutyl)-8-(methylamino)imidazo[1,2-b]pyridazine-3-carboxamide 2,2,2-trifluoroacetate (Intermediate ML)

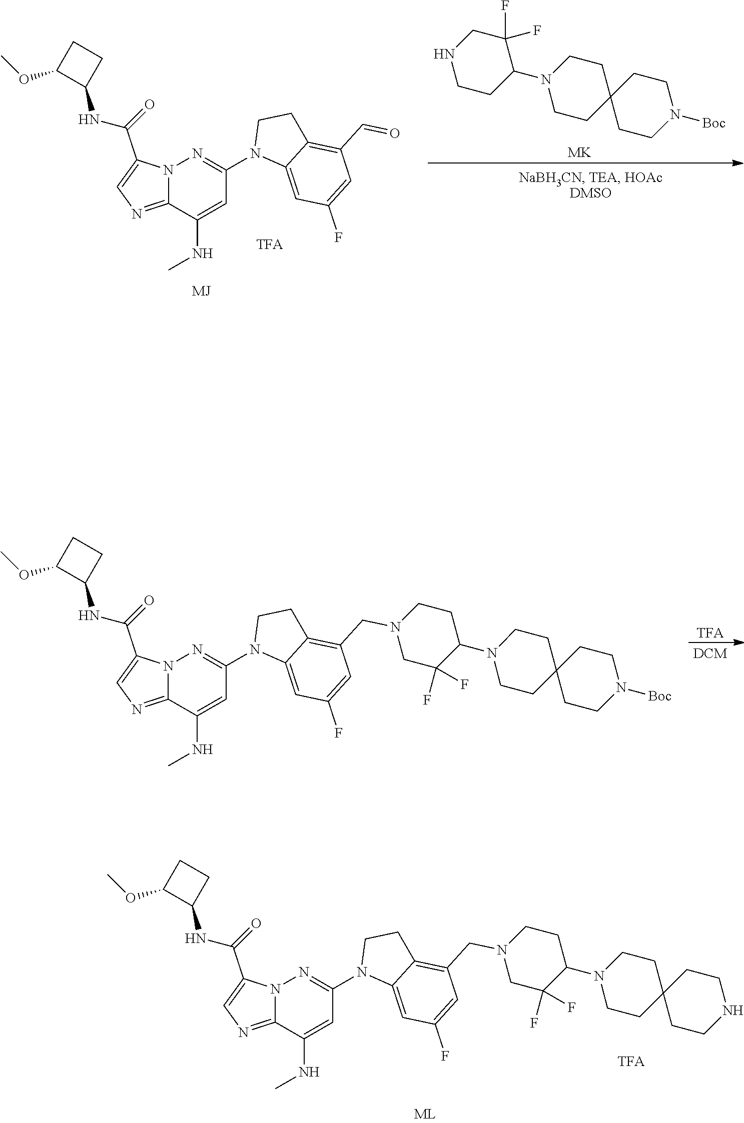

Step 1—Tert-butyl 9-(3,3-difluoro-1-((6-fluoro-1-(3-(((1R,2R)-2-methoxycyclobutyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate. To a stirred solution of 6-(6-fluoro-4-formylindolin-1-yl)-N-((1R,2R)-2-methoxycyclobutyl)-8-(methylamino)imidazo[1,2-b]pyridazine-3-carboxamide 2,2,2-trifluoroacetate (550 mg, 1.25 mmol, Intermediate MJ), tert-butyl 9-(3,3-difluoropiperidin-4-yl)-3,9-diazaspiro[5.5]undecane-3-carboxylate (468 mg, 1.25 mmol, Intermediate MK) in DMSO (8 mL) were added TEA (127 mg, 1.25 mmol) and HOAc (226 mg, 3.76 mmol) at rt under air atmosphere. The resulting mixture was stirred for 1 h at 50° C. under air atmosphere. Next, NaBH₃CN (315 mg, 5.02 mmol) was added to the mixture in portions at rt under air atmosphere. The resulting mixture was stirred for 1 h at 50° C. under air atmosphere. On completion, the mixture was cooled to rt and purified by reversed-phase flash chromatography (column, C18 silica gel; mobile phase, MeCN in Water (10 mmol/L NH4HCO₃), 50% to 95% gradient in 30 min; detector, UV 254 nm) to afford the title compound (500 mg, 50% yield) as a light brown solid. LC/MS (ESI, m/z): [(M+1)]⁺=796.3.

Step 2—6-(4-((3,3-Difluoro-4-(3,9-diazaspiro[5.5]undecan-3-yl)piperidin-1-yl)methyl)-6-fluoroindolin-1-yl)-N-((1R,2R)-2-methoxycyclobutyl)-8-(methylamino)imidazo[1,2-b]pyridazine-3-carboxamide 2,2,2-trifluoroacetate. To a stirred solution of tert-butyl 9-(3,3-difluoro-1-((6-fluoro-1-(3-(((1R,2R)-2-methoxycyclobutyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate (500 mg, 0.63 mmol) in DCM (12 mL) was added TFA (4 mL) at rt under air atmosphere. The resulting mixture was stirred for 1 h at rt under air atmosphere. On completion, the mixture was concentrated under vacuum. The residue was purified by trituration with ethyl ether (50 mL). The resulting mixture was dried under vacuum to afford the title compound (500 mg, TFA salt) as a light brown solid. LC/MS (ESI, m/z): [(M+1)]⁺=696.3.

### 4-Chloro-3-(2,4-dioxotetrahydropyrimidin-1(2H)-yl)benzoic acid (Intermediate MM)

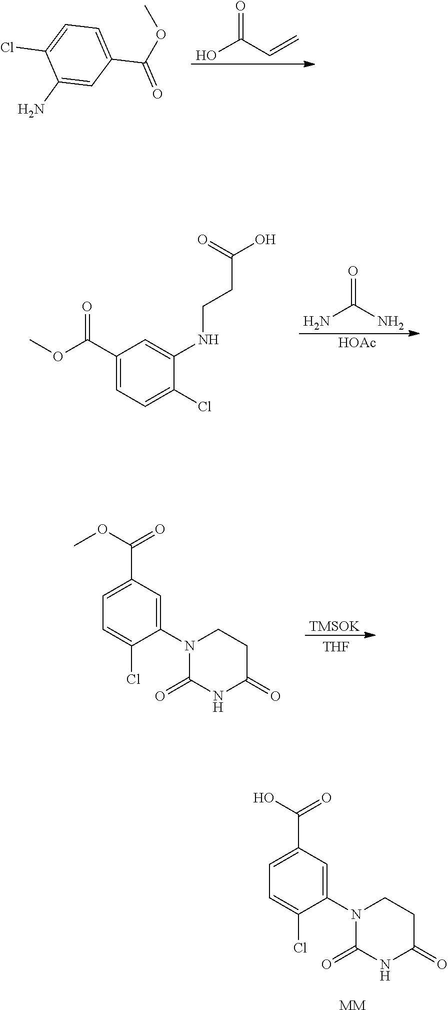

Step 1—3-{[2-Chloro-5-(methoxycarbonyl)phenyl]amino}propanoic acid. Into a 1000 mL round-bottom flask were added methyl 3-amino-4-chlorobenzoate (25 g, 140 mmol) and acrylic acid (200 mL) at rt. The resulting mixture was stirred for 4 h at 100° C. under nitrogen atmosphere. On completion, the reaction mixture was cooled to rt then diluted with water (2000 mL). The resulting mixture was extracted with EA (3×500 mL). The combined organic layers were washed with brine (3×200 mL), and dried over anhydrous Na₂SO₄. After filtration, the filtrate was concentrated under reduced pressure to give the title compound (25 g) as a yellow oil. LC/MS (ESI, m/z): [(M+H)]⁺=258.1.

Step 2—Methyl 4-chloro-3-(2,4-dioxo-1,3-diazinan-1-yl)benzoate. To a stirred solution of 3-{[2-chloro-5-(methoxycarbonyl)phenyl]amino}propanoic acid (25 g, 97 mmol) in AcOH (200 mL) was added urea (40.79 g, 679.2 mmol) at rt under nitrogen atmosphere. The resulting mixture was stirred for 16 h at 110° C. under nitrogen atmosphere. On completion, the reaction mixture was cooled to rt and diluted with water (2000 mL). The resulting mixture was extracted with EA (3×500 mL). The combined organic layers were washed with brine (3×300 mL), and dried over anhydrous Na₂SO₄. After filtration, the filtrate was concentrated under reduced pressure. The residue was purified by trituration with MTBE (300 mL). The resulting mixture was filtered, and the filter cake was washed with MTBE (3×10 mL) was dried under reduced pressure to give the title compound (14 g, 51% yield) as a white solid. LC/MS (ESI, m/z): [(M+H)]⁺=283.1.

Step 3—4-Chloro-3-(2,4-dioxo-1,3-diazinan-1-yl)benzoic acid. To a stirred solution of methyl 4-chloro-3-(2,4-dioxo-1,3-diazinan-1-yl)benzoate (5 g, 20 mmol) in THF (100 mL) was added trimethyl(potassiooxy)silane (4.54 g, 35.4 mmol) at rt under nitrogen atmosphere. The resulting mixture was stirred for 2 h at rt under nitrogen atmosphere. On completion, the mixture was acidified to pH 4 with formic acid and the mixture was concentrated under reduced pressure. The residue was purified by reverse flash chromatography (column: C18 silica gel; mobile phase, MeCN in Water (0.1% FA), 15% to 45% gradient in 30 min; detector, UV 254 nm, desired fractions were collected at 23% B) and concentrated under reduced pressure to afford the title compound (2 g, 42% yield) as a white solid. LC/MS (ESI, m/z): [(M+H)]⁺=269.0.

### 6-(4-Formylindolin-1-yl)-N-((1R,2R)-2-methoxycyclobutyl)-8-(methylamino)imidazo[1,2-b]pyridazine-3-carboxamide 2,2,2-trifluoroacetate (Intermediate MN)

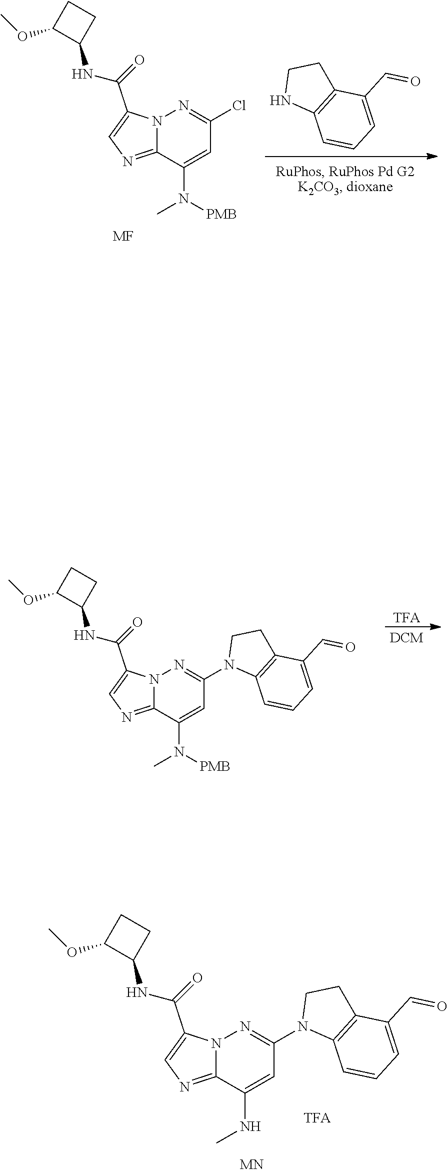

Step 1—6-(4-Formylindolin-1-yl)-8-((4-methoxybenzyl)(methyl)amino)-N-((1R,2R)-2-methoxycyclobutyl)imidazo[1,2-b]pyridazine-3-carboxamide. To a stirred mixture of 6-chloro-8-((4-methoxybenzyl)(methyl)amino)-N-((1R,2R)-2-methoxycyclobutyl)imidazo[1,2-b]pyridazine-3-carboxamide (200 mg, 0.5 mmol, Intermediate MF), indoline-4-carbaldehyde (68 mg, 0.465 mmol, CAS #1074-86-8) and K₂CO₃ (193 mg, 1.40 mmol) in dioxane (4 mL) were added RuPhos (22 mg, 0.047 mmol) and RuPhos-PdCl-2nd G (36 mg, 0.047 mmol) at rt under nitrogen atmosphere. The resulting mixture was stirred for 2 h at 100° C. under nitrogen atmosphere. On completion, the mixture was cooled to rt and purified by reversed-phase flash chromatography (Column: Spherical C18, 20-40 μm, 120 g; Mobile Phase A: Water (plus 10 mM NH4HCO₃), Mobile Phase B: ACN; Flow rate: 60 mL/min; Gradient (B %): 5%˜50%, 4 min; 50%˜85%, 20 min; 85%˜95%; 2 min; 95%, 5 min; Detector: 254 nm; the fractions containing desired product were collected at 78% B) and concentrated under reduced pressure to afford the title compound (170 mg, 67% yield) as a yellow solid. LC/MS (ESI, m/z): [(M+1)]⁺=541.4.

Step 2—6-(4-Formylindolin-1-yl)-N-((1R,2R)-2-methoxycyclobutyl)-8-(methylamino)imidazo[1,2-b]pyridazine-3-carboxamide 2,2,2-trifluoroacetate. A solution of 6-(4-formylindolin-1-yl)-8-((4-methoxybenzyl)(methyl)amino)-N-((1R,2R)-2-methoxycyclobutyl)imidazo[1,2-b]pyridazine-3-carboxamide (170 mg, 0.314 mmol) in DCM (4 mL) and TFA (2 mL) was stirred for 1 h at rt. On completion, the mixture was concentrated under reduced pressure. The resulting mixture was diluted with diethyl ether (20 mL). The precipitated solids were collected by filtration and washed with diethyl ether (2×10 mL) to give the title compound (170 mg, TFA salt) as a yellow solid. LC/MS (ESI, m/z): [(M+1)]⁺=421.3.

### 6-(4-((3,3-Difluoro-4-(3,9-diazaspiro[5.5]undecan-3-yl)piperidin-1-yl)methyl)indolin-1-yl)-N-((1R,2R)-2-methoxycyclobutyl)-8-(methylamino)imidazo[1,2-b]pyridazine-3-carboxamide 2,2,2-trifluoroacetate (Intermediate MO)

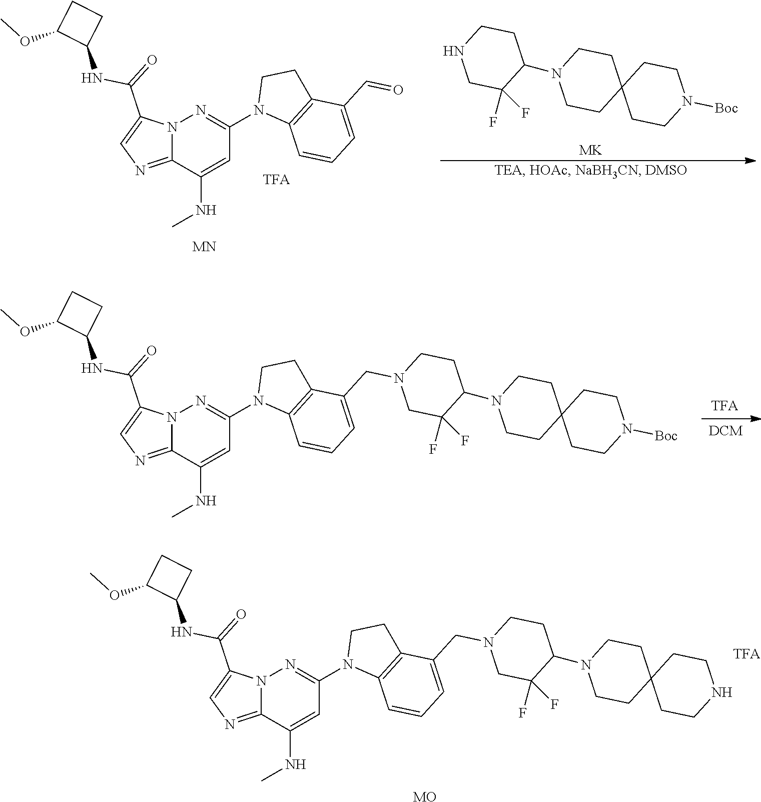

Step 1—Tert-butyl 9-(3,3-difluoro-1-((1-(3-(((1R,2R)-2-methoxycyclobutyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate. To a stirred solution of 6-(4-formylindolin-1-yl)-N-((1R,2R)-2-methoxycyclobutyl)-8-(methylamino)imidazo[1,2-b]pyridazine-3-carboxamide 2,2,2-trifluoroacetate (170 mg, 0.318 mmol, Intermediate MN) and TEA (64 mg, 0.636 mmol) in DMSO (4 mL) were added tert-butyl 9-(3,3-difluoropiperidin-4-yl)-3,9-diazaspiro[5.5]undecane-3-carboxylate (119 mg, 0.318 mmol, Intermediate MK) and AcOH (191 mg, 3.180 mmol) at rt. The resulting mixture was stirred for 1 h at 50° C. To the above mixture was added NaBH₃CN (100 mg, 1.590 mmol) at rt and the mixture was stirred for additional 1 h at 50° C. On completion, the mixture was cooled to it and the mixture was purified by reversed-phase flash chromatography (Column: Spherical C18, 20˜40 m, 120 g; Mobile Phase A: Water (plus 10 mM NH₄HCO₃). Mobile Phase B: ACN; Flow rate: 50 mL/min; Gradient (B %): 5%˜40%, 4 min; 40%˜70%, 20 min; 70%˜95%; 2 min; 95%, 5 min; Detector: 254 nm; the fractions containing desired product were collected at 66% B) and concentrated under reduced pressure to afford the title compound (190 mg, 76% yield) as a yellow solid. LC/MS (ESI, m/z): [(M+1)]⁻=778.6.

Step 2—6-(4-((3,3-Difluoro-4-(3,9-diazaspiro[5.5]undecan-3-yl)piperidin-1-yl)methyl)indolin-1-yl)-N-((1R,2R)-2-methoxycyclobutyl)-8-(methylamino)imidazo[1,2-b]pyridazine-3-carboxamide 2,2,2-trifluoroacetate. A solution of tert-butyl 9-(3,3-difluoro-1-((1-(3-(((1R,2R)-2-methoxycyclobutyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate (190 mg, 0.244 mmol) in DCM (5 mL) and TFA (1 mL) was stirred for 1 h at rt. On completion, the mixture was concentrated under reduced pressure and diluted with diethyl ether (20 mL). The precipitated solids were collected by filtration and washed with diethyl ether (2×10 mL) to give the title compound (220 mg, TFA salt) as an off-white solid. LC/MS (ESI, m/z): [(M+1)]⁺=678.6.

### Tert-butyl 9-[(4R)-3,3-difluoropiperidin-4-yl]-3,9-diazaspiro[5.5]undecane-3-carboxylate (Intermediate MP) and tert-butyl 9-[(4S)-3,3-difluoropiperidin-4-yl]-3,9-diazaspiro[5.5]undecane-3-carboxylate (Intermediate MQ)

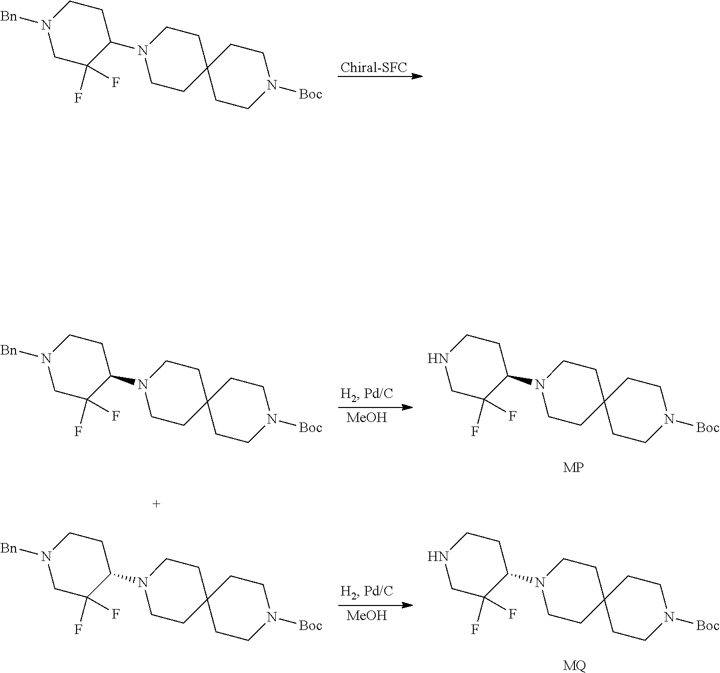

Step 1—Tert-butyl 9-[(4R)-1-benzyl-3,3-difluoropiperidin-4-yl]-3,9-diazaspiro[5.5]undecane-3-carboxylate and tert-butyl 9-[(4S)-1-benzyl-3,3-difluoropiperidin-4-yl]-3,9-diazaspiro[5.5]undecane-3-carboxylate. Racemic tert-butyl 9-(1-benzyl-3,3-difluoropiperidin-4-yl)-3,9-diazaspiro[5.5]undecane-3-carboxylate (110 g, synthesized via Step 1 of Intermediate MK) was separated by chiral resolution (Column: CHIRALPAK IG, 5*25 cm, 10 μm; Mobile Phase A: CO₂, Mobile Phase B: IPA(0.1% DEA); Flow rate: 100 mL/min; Gradient: isocratic 30% B; Back Pressure(bar): 100; Wave Length: 220 nm; RT1(min): 6.74; RT2(min): 8.75. The fractions collected at 6.74 min (faster eluting peak) were concentrated under vacuum to afford tert-butyl 9-[(4R)-1-benzyl-3,3-difluoropiperidin-4-yl]-3,9-diazaspiro[5.5]undecane-3-carboxylate (47 g, 43% yield) as a white solid. The fractions collected at 8.75 min (slower eluting peak) were concentrated under vacuum tert-butyl 9-[(4S)-1-benzyl-3,3-difluoropiperidin-4-yl]-3,9-diazaspiro[5.5]undecane-3-carboxylate (39 g, 35% yield) as a white solid. LC/MS (ESI, m/z): [(M+H)]⁺=374.3 for both isomers. The absolute stereochemistry of the enantiomers was assigned arbitrarily.

Step 2—Tert-butyl 9-[(4R)-3,3-difluoropiperidin-4-yl]-3,9-diazaspiro[5.5]undecane-3-carboxylate. To a solution of tert-butyl 9-[(4R)-1-benzyl-3,3-difluoropiperidin-4-yl]-3,9-diazaspiro[5.5]undecane-3-carboxylate (2.7 g, 5.8 mmol) in MeOH (50 mL) was added Pd/C (10 wt %, 1.5 g) under nitrogen atmosphere in a 250 mL round-bottom flask. The mixture was hydrogenated at rt for 2 h under hydrogen atmosphere using a hydrogen balloon. On completion, the mixture was filtered through a Celite pad and concentrated under reduced pressure to give the title compound (1.7 g, 78% yield) as an off-white solid. ¹H NMR (400 MHz, DMSO-d₆) δ 3.30-3.24 (m, 4H), 2.99-2.87 (m, 2H), 2.86-2.73 (m, 1H), 2.72-2.60 (m, 5H), 2.50-2.42 (m, 1H), 2.28 (s, 1H), 1.74-1.60 (m, 2H), 1.43-1.37 (m, 13H), 1.35-1.29 (m, 4H); LC/MS (ESI, m/z): [(M+H)]⁺=374.3.

Step 3—Tert-butyl 9-[(4S)-3,3-difluoropiperidin-4-yl]-3,9-diazaspiro[5.5]undecane-3-carboxylate. To a stirred solution of tert-butyl 9-[(4S)-1-benzyl-3,3-difluoropiperidin-4-yl]-3,9-diazaspiro[5.5]undecane-3-carboxylate (8 g, 20 mmol) in MeOH (160 mL) was added Pd/C (10 wt %, 2.4 g) at rt under nitrogen atmosphere. The resulting mixture was stirred for 1 h at rt under hydrogen atmosphere. On completion, the mixture was filtered, and the filter cake was washed with MeOH (3×50 mL). The filtrate was concentrated under reduced pressure to afford the title compound (6.2 g, 96% yield) as a grey solid. ¹H NMR (300 MHz, DMSO-d₆) δ 3.31-3.25 (m, 4H), 3.00-2.86 (m, 2H), 2.86-2.75 (m, 1H), 2.74-2.59 (m, 5H), 2.48-2.39 (m, 1H), 2.31 (s, 1H), 1.76-1.60 (m, 2H), 1.46-1.37 (m, 13H), 1.36-1.28 (m, 4H); LC/MS (ESI, m/z): [(M+H)]⁺=374.3.

### 6-(4-(((S)-3,3-difluoro-4-(3,9-diazaspiro[5.5]undecan-3-yl)piperidin-1-yl)methyl)indolin-1-yl)-N-((1R,2R)-2-methoxycyclobutyl)-8-(methylamino)imidazo[1,2-b]pyridazine-3-carboxamide trifluoroacetate (Intermediate MR)

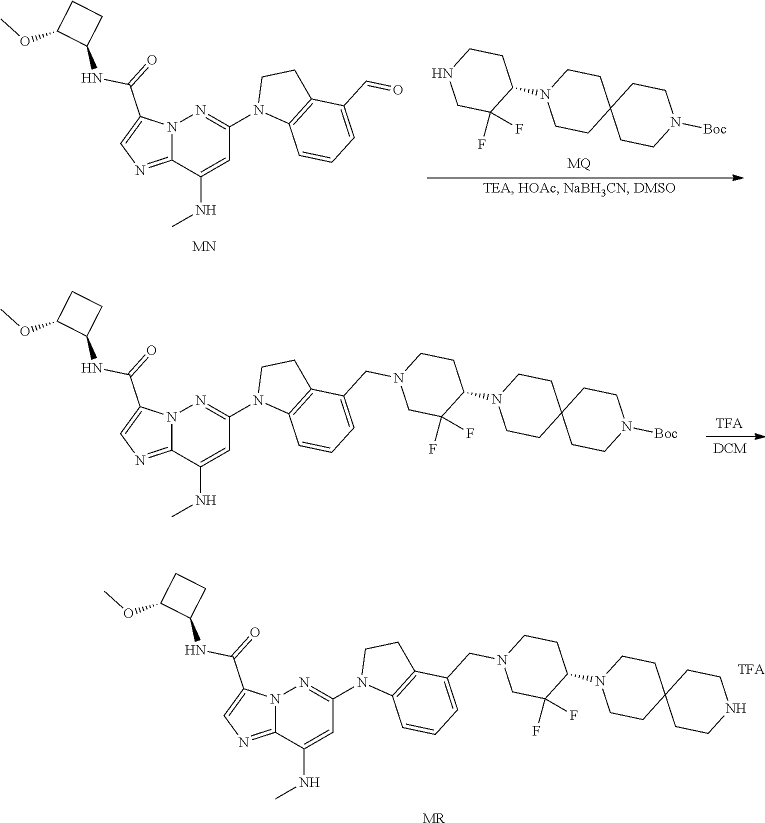

Step 1—Tert-butyl 9-((S)-3,3-difluoro-1-((1-(3-(((1R,2R)-2-methoxycyclobutyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate. To a stirred mixture of tert-butyl (S)-9-(3,3-difluoropiperidin-4-yl)-3,9-diazaspiro[5.5]undecane-3-carboxylate (864 mg, 2.32 mmol, Intermediate MQ) and TEA (98 mg, 0.97 mmol) in DMSO (20 mL) were added 6-(4-formylindolin-1-yl)-N-((1R,2R)-2-methoxycyclobutyl)-8-(methylamino)imidazo[1,2-b]pyridazine-3-carboxamide 2,2,2-trifluoroacetate (1.00 g, 1.93 mmol, Intermediate MN) and AcOH (463 mg, 7.72 mmol) at rt. The resulting mixture was stirred for 1 h at 50° C. To the above mixture was added NaBH₃CN (486 mg, 7.724 mmol) at rt and the mixture was stirred for an additional 1 h at 50° C. On completion, the mixture was cooled to rt and was purified by reverse phase flash chromatography (Column: WelFlash™ C18-I, 20-40 μm, 330 g; Eluent A: Water (plus 10 mmol/L NH4HCO₃); Eluent B: ACN; Gradient: 65%-98% B in 25 min; Flow rate: 80 mL/min; Detector: 220 nm; desired fractions were collected at 98% B) and concentrated under reduced pressure to afford the title compound (1 g, 66%) as a light yellow solid. LC/IS (ESI, m/z): [(M+H)]⁺=778.5.

Step 2—6-(4-(((S)-3,3-Difluoro-4-(3,9-diazaspiro[5.5]undecan-3-yl)piperidin-1-yl)methyl)indolin-1-yl)-N-((1R,2R)-2-methoxycyclobutyl)-8-(methylamino)imidazo[1,2-b]pyridazine-3-carboxamide trifluoroacetate. To a stirred mixture of tert-butyl 9-((S)-3,3-difluoro-1-((1-(3-(((1R,2R)-2-methoxycyclobutyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate (1.00 g, 1.28 mmol) in DCM (15 mL) was added TFA (5.00 mL). The resulting mixture was stirred for 1 h at rt. On completion, the mixture was concentrated under reduced pressure. The residue was purified by trituration with diethyl ether (20 mL) to give the title compound (1.1 g, TFA salt) as a yellow solid. LC/MS (ESI, m/z): [(M+H)]⁺=678.5.

### 3-Chloro-5-(2,4-dioxotetrahydropyrimidin-1(2H)-yl)-4-methylbenzoic acid (Intermediate MS)

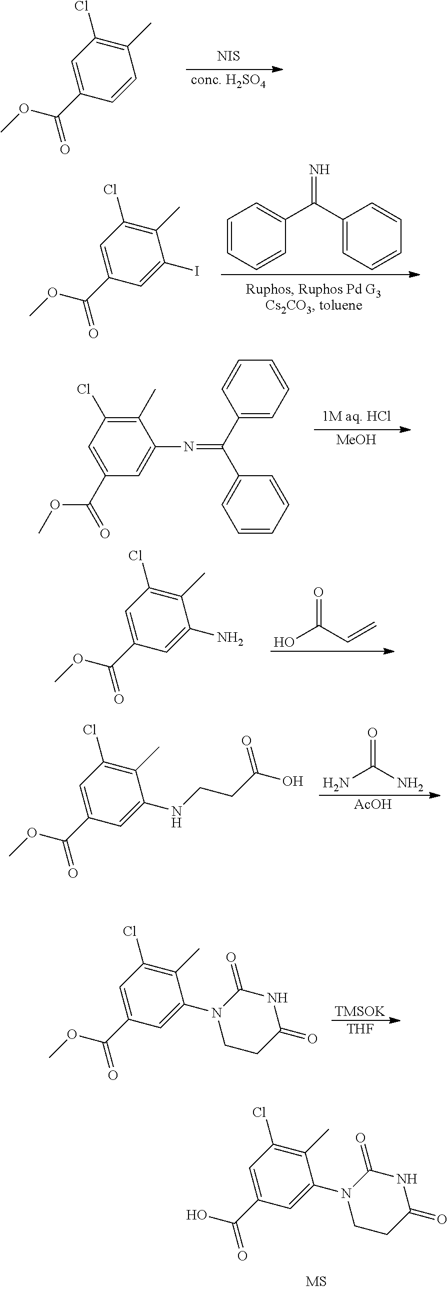

Step 1—Methyl 3-chloro-5-iodo-4-methylbenzoate. To a stirred mixture of methyl₃-chloro-4-methylbenzoate (400 g, 2.17 mol, CAS #56525-63-4) in H₂SO₄ (2000 mL) was added NIS (584.95 g, 2.6 mol) at rt. The resulting mixture was stirred for 2 h at rt. On completion, the mixture was diluted with water (4000 mL) and extracted with EtOAc (3×300 mL). The combined organic layers were dried over anhydrous Na₂SO₄, filtered, and the filtrate was concentrated under reduced pressure. The residue was purified by silica gel column chromatography, eluted with PE/EA (15:1˜10:1), to afford the title compound (464 g, 69% yield) as a white solid. ¹H NMR (300 MHz, DMSO-d₆) δ 8.27-8.22 (m, 111), 7.901-7.87 (m, 111), 3.86 (s, 3H), 2.57 (s, 3H).

Step 2—Methyl 3-chloro-5-[(diphenylmethylidene)amino]-4-methylbenzoate. To a stirred mixture of methyl 3-chloro-5-iodo-4-methylbenzoate (30 g, 100 mmol) and diphenylmethanimine (21.01 g, 115.9 mmol) in toluene (350 mL) were added RuPhos Palladacycle Gen.3 (8.08 g, 9.66 mmol), RuPhos (4.51 g, 9.66 mmol) and Cs₂CO₃ (62.96 g, 193.2 mmol) at it under nitrogen atmosphere. The resulting mixture was stirred for 2 h at 100° C. under nitrogen atmosphere. On completion, the mixture was cooled to it and concentrated under reduced pressure. The residue was purified by silica gel column chromatography, eluted with PE/EA, (10:1˜5:1) to afford the title compound (18 g, 51% yield) as a yellow oil. LC/MS (ESI, m/z): [(M+H)]⁺=364.1.

Step 3—Methyl 3-amino-5-chloro-4-methylbenzoate. To a stirred solution of methyl 3-chloro-5-[(diphenylmethylidene)amino]-4-methylbenzoate (18 g, 50 mmol) in MeOH (600 mL) was added and 1M aq. HCl (500 mL) dropwise at rt. The resulting mixture was stirred for 1 h at 50° C. On completion, the mixture was cooled to rt and concentrated under reduced pressure. The resulting mixture was diluted with water (200 mL) and extracted with EtOAc (3×400 mL). The combined organic layers were dried over anhydrous Na₂SO₄, filtered, and the filtrate was concentrated under reduced pressure. The residue was purified by silica gel column chromatography, eluted with PE/EA (10:1˜4:1), to afford the title compound (6.2 g, 63% yield) as a yellow solid. H NMR (300 MHz, Chloroform-d) δ 7.61 (s, 1H), 7.42 (s, 1H), 3.91 (s, 3H), 2.36 (s, 3H). LC/MS (ESI, m/z): [(M+H)]⁺=200.0.

Step 4—3-{[3-Chloro-5-(methoxycarbonyl)-2-methylphenyl]amino}propanoic acid. Into a 250 mL round-bottom flask were added methyl 3-amino-5-chloro-4-methylbenzoate (6.2 g, 31 mmol) and acrylic acid (4.48 g, 62.1 mmol) at rt. The resulting mixture was stirred for 2 h at 100° C. On completion, the mixture was cooled to rt and diluted with water (1500 mL). The resulting mixture was extracted with EtOAc (3×500 mL). The combined organic layers were dried over anhydrous Na₂SO₄, filtered, and the filtrate was concentrated under reduced pressure to give the tide compound (10 g) as a brown solid. LC/MS (ESI, m/z): [(M+H)]⁺=272.1.

Step 5—Methyl 3-chloro-5-(2,4-dioxo-1,3-diazinan-1-yl)-4-methylbenzoate. A stirred mixture of 3-{[3-chloro-5-(methoxycarbonyl)-2-methylphenyl]amino}propanoic acid (10 g, 36.805 mmol) and urea (15.47 g, 257.6 mmol) in HOAc (500 mL) was stirred for 16 h at 100° C. On completion, the mixture was cooled to rt and the solution was poured into ice/water. The precipitated solids were collected by filtration and washed with water (3×30 mL) and acetonitrile (2×30 mL). Then the solid was dried under reduced pressure to give the title compound (7.4 g, 68% yield) as a yellow solid. ¹H NMR (400 MHz, Chloroform-d) δ 8.07 (d, J=1.7 Hz, 1H), 7.82 (d, J=1.7 Hz, 1H), 7.66 (s, 1H), 3.94 (s, 3H), 3.90 (dt, J=12.5, 7.3 Hz, 1H), 3.67 (dt, J=12.4, 6.1 Hz, 1H), 2.90 (dd, J=7.4, 6.1 Hz, 2H), 2.38 (s, 3H); LC/MS (ESI, m/z): [(M+H)]⁺=297.1.

Step 6—3-Chloro-5-(2,4-dioxo-1,3-diazinan-1-yl)-4-methylbenzoic acid. A mixture of methyl 3-chloro-5-(2,4-dioxo-1,3-diazinan-1-yl)-4-methylbenzoate (7 g, 20 mmol) and trimethyl(potassiooxy)silane (9.08 g, 70.8 mmol) in THF (800 mL) was stirred for 1 h at rt. On completion, the mixture was poured 2 N HCl slowly. The precipitated solids were collected by filtration and washed with water (3×20 mL). Then the solid was dried under reduced pressure to give the title compound (6.5 g, 97% yield) as an off-white solid. ¹H NMR (400 MHz, DMSO-d₆) δ 13.35 (s, 1H), 10.46 (s, 1H), 7.89 (d, J=1.7 Hz, 1H), 7.86 (d, J=1.7 Hz, 1H), 3.90-3.83 (m, 1H), 3.61-3.54 (m, 1H), 2.87-2.79 (m, 1H), 2.76-2.67 (m, 1H), 2.28 (s, 3H); LC/MS (ESI, m/z): [(M+H)]⁺=283.0.

### Tert-butyl 4-{3,9-diazaspiro[5.5]undecan-3-ylmethyl}-3,3-difluoropiperidine-1-carboxylate (Intermediate MT)

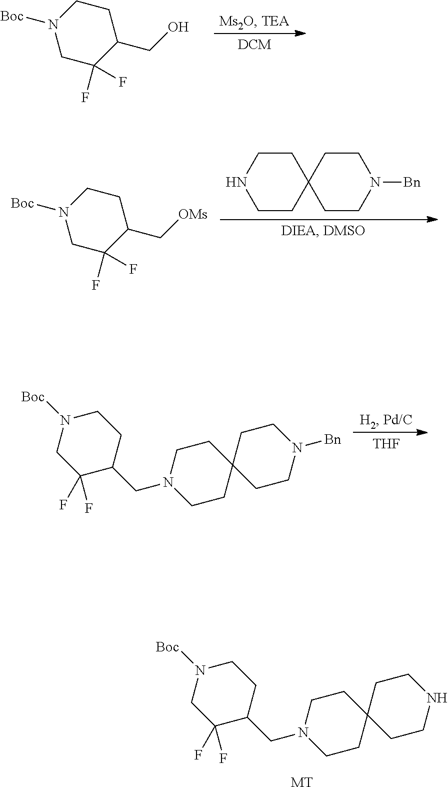

Step 1—Tert-butyl 3,3-difluoro-4-[(methanesulfonyloxv)methyl]piperidine-1-carboxylate. To a stirred mixture of tert-butyl 3,3-difluoro-4-(hydroxymethyl)piperidine-1-carboxylate (3 g, 10 mmol, CAS #1303974-47-1) and TEA (4.97 mL, 35.817 mmol) in DCM (50 mL) was added methanesulfonyl methanesulfonate (3.12 g, 17.9 mmol) at rt. The resulting mixture was stirred for 1 h at rt. On completion, the mixture was concentrated under vacuum. The residue was purified by silica gel column chromatography, eluted with PE/EA (1:1), to afford the title compound (5 g) as a yellow oil. LC/MS (ESI, m/z): [(M+H -56)]⁺=274.0.

Step 2—Tert-butyl 4-({9-benzyl-3,9-diazaspiro[5.5]undecan-3-yl}methyl)-3,3-difluoropiperidine-1-carboxylate. To a stirred mixture of tert-butyl 3,3-difluoro-4-[(methanesulfonyloxy)methyl]piperidine-1-carboxylate (1.5 g, 4.554 mmol) and 3-benzyl-3,9-diazaspiro[5.5]undecane (1.67 g, 6.83 mmol, CAS #189333-49-1) in DMSO (15 mL) was added DIEA (3.17 mL, 18.2 mmol) at rt under nitrogen atmosphere. The resulting mixture was stirred for 1 h at 120° C. under nitrogen atmosphere. On completion, the mixture was cooled to rt and concentrated under vacuum. The residue was purified by reverse phase flash chromatography (Column: WelFlash™ C18-I, 20-40 μm, 330 g; Eluent A: Water (plus 10 mmol/L NH4HCO₃); Eluent B: ACN; Gradient: 75%-98% B in 25 min; Flow rate: 80 mL/min; Detector: 220/254 nm; desired fractions were collected at 95% B) and concentrated under reduced pressure to afford the title compound (750 mg, 35% yield) as a yellow oil. LC/MS (ESI, m/z): [(M+H)]⁺=478.3.

Step 3—Tert-butyl 4-{3,9-diazaspiro[5.5]undecan-3-ylmethyl}-3,3-difluoropiperidine-1-carboxylate. To a solution of tert-butyl 4-({9-benzyl-3,9-diazaspiro[5.5]undecan-3-yl}methyl)-3,3-difluoropiperidine-1-carboxylate (750 mg, 1.57 mmol) in THF (15 mL) was added Pd/C (10 wt %, 300 mg) under nitrogen atmosphere. The mixture was hydrogenated at rt for 2 h under hydrogen atmosphere using a hydrogen balloon. On completion, the mixture was filtered through a Celite pad and concentrated under reduced pressure to give the title compound (400 mg, 66% yield) as a black oil. LC/MS (ESI, m/z): [(M+H)]⁺=388.2.

### 1-(2-Chloro-5-{9-[(3,3-difluoropiperidin-4-yl)methyl]-3,9-diazaspiro[5.5]undecane-3-carbonyl}phenyl)-1,3-diazinane-2,4-dione trifluoroacetate (Intermediate MU)

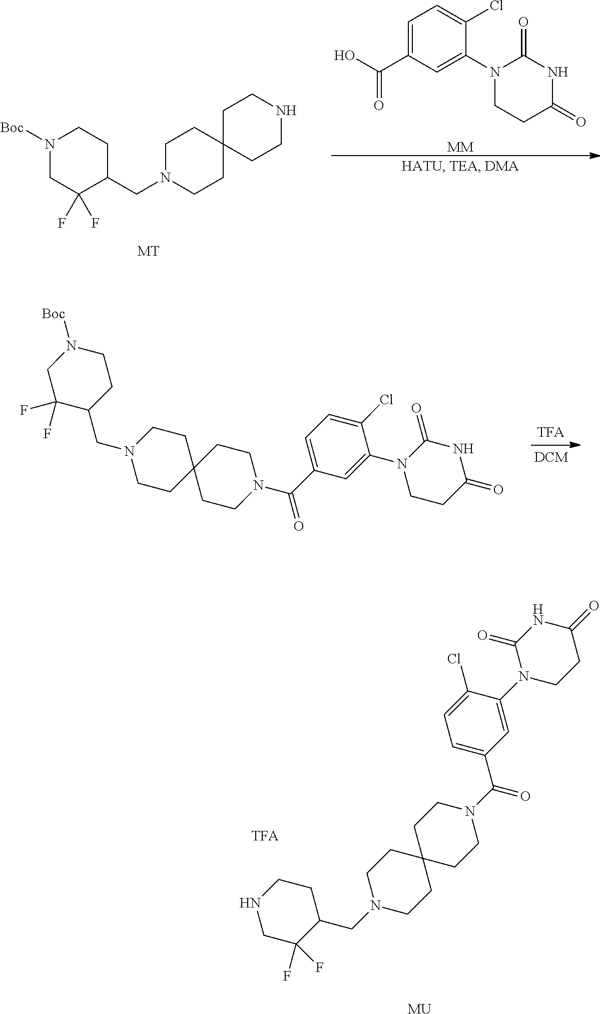

Step 1—Tert-butyl 4-({9-[4-chloro-3-(2,4-dioxo-1,3-diazinan-1-yl)benzoyl]-3,9-diazaspiro[5.5]undecan-3-yl}methyl)-3,3-difluoropiperidine-1-carboxylate. To a stirred mixture of tert-butyl 4-{3,9-diazaspiro[5.5]undecan-3-ylmethyl}-3,3-difluoropiperidine-1-carboxylate (400 mg, 1.03 mmol, Intermediate MT) and 4-chloro-3-(2,4-dioxo-1,3-diazinan-1-yl)benzoic acid (277.30 mg, 1.032 mmol, Intermediate MM) in DMA (5 mL) were added TEA (0.43 mL, 3.10 mmol) and HATU (588.73 mg, 1.548 mmol) at rt under nitrogen atmosphere. The resulting mixture was stirred for 1 h at rt under nitrogen atmosphere. On completion, the mixture was concentrated under vacuum. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 45%-85% B in 25 min; Flow rate: 80 mL/min; Detector: 220/254 nm; desired fractions were collected at 70% B) and concentrated under reduced pressure to afford the title compound (300 mg, 46% yield) as a white solid. LC/MS (ESI, m/z): [(M+H)]⁺=638.1.

Step 2—1-(2-Chloro-5-{9-[(3,3-difluoropiperidin-4-yl)methyl]-3,9-diazaspiro[5.5]undecane-3-carbonyl}phenyl)-1,3-diazinane-2,4-dione trifluoroacetate. To a stirred solution of tert-butyl 4-({9-[4-chloro-3-(2,4-dioxo-1,3-diazinan-1-yl)benzoyl]-3,9-diazaspiro[5.5]undecan-3-yl}methyl)-3,3-difluoropiperidine-1-carboxylate (300 mg, 0.470 mmol) in DCM (6 mL) was added TFA (3 mL) at rt under nitrogen atmosphere and the mixture was stirred for 1 h at rt under nitrogen atmosphere. On completion, the mixture was concentrated under vacuum. The residue was purified by trituration with Et2O (20 mL) to afford the title compound (290 mg, 97% yield) as a white solid. LC/MS (ESI, m/z): [(M+H)]⁺=538.2.

### Tert-butyl 1′-(3,3-difluoropiperidin-4-yl)-[4,4′-bipiperidine]-1-carboxylate (Intermediate MV)

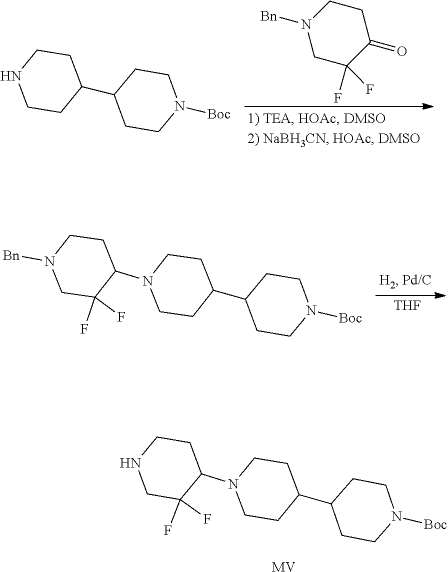

Step 1—Tert-butyl 1′-(1-benzyl-3,3-difluoro-2,6-dihydropyridin-4-yl)-[4,4′-bipiperidine]-1-carboxylate. To a stirred solution of tert-butyl [4,4′-bipiperidine]-1-carboxylate (6 g, 20 mmol, CAS #171049-35-7) in DMSO (60 mL) was added TEA (9.32 mL, 67.1 mmol) at rt under nitrogen atmosphere. The resulting mixture was stirred for 5 min at it under nitrogen atmosphere. To the above mixture was added 1-benzyl-3,3-difluoropiperidin-4-one (5.04 g, 22.4 mmol) and HOAc (6.40 mL, 112 mmol) at rt. The resulting mixture was stirred for an additional 2 h at 100° C. Next, the mixture was cooled to rt and added MeOH (60 mL), HOAc (6 mL) and NaBH₃CN (1.59 g, 25.230 mmol) was added at rt under nitrogen atmosphere. The resulting mixture was stirred for 2 h at 50° C. under nitrogen atmosphere. On completion, the mixture was cooled to rt and concentrated under reduced pressure. The residue was purified by reverse flash chromatography (column, C18 silica gel; mobile phase A: water (10 mmol/L NH₄HCO₃), mobile phase B: ACN, 60% to 100% gradient in 30 min; detector, UV 254 nm; the fractions were collected at 100%) and concentrated under reduced pressure to afford the title compound (5.2 g, 86% yield) as a yellow oil. LC/MS (ESI, m/z): [(M+H)]⁺=478.4.

Step 2—Tert-butyl 1′-(3,3-difluoropiperidin-4-yl)-[4,4′-bipiperidine]-1-carboxylate. To a stirred solution of tert-butyl 1′-(1-benzyl-3,3-difluoropiperidin-4-yl)-[4,4′-bipiperidine]-1-carboxylate (5 g, 10 mmol) in THF (70 mL) was added Pd/C (5.57 g, 52.3 mmol) at rt. The resulting mixture was purged with hydrogen three times and stirred for 2 h at rt under hydrogen. On completion, the mixture was filtered, and the filter cake was washed with THF (3×30 mL). The filtrate was concentrated under reduced pressure. The residue was purified by reverse flash chromatography (column, C18 silica gel; mobile phase A: water (10 mmol/L NH₄HCO₃), mobile phase B: ACN, 40% to 70% gradient in 30 min; detector, UV 254 nm; the fractions were collected at 45%) and concentrated under reduced pressure to afford the title compound (3 g, 74% yield) as a white solid. LC/MS (ESI, m/z): [(M+H)]⁺=388.7.

### 6-{4-[(4-{[4,4′-Bipiperidin]-1-yl}-3,3-difluoropiperidin-1-yl)methyl]-2,3-dihydroindol-1-yl}-N-[(1R,2R)-2-methoxycyclobutyl]-8-(methylamino)imidazo[1,2-b]pyridazine-3-carboxamide trifluoroacetate (Intermediate MW)

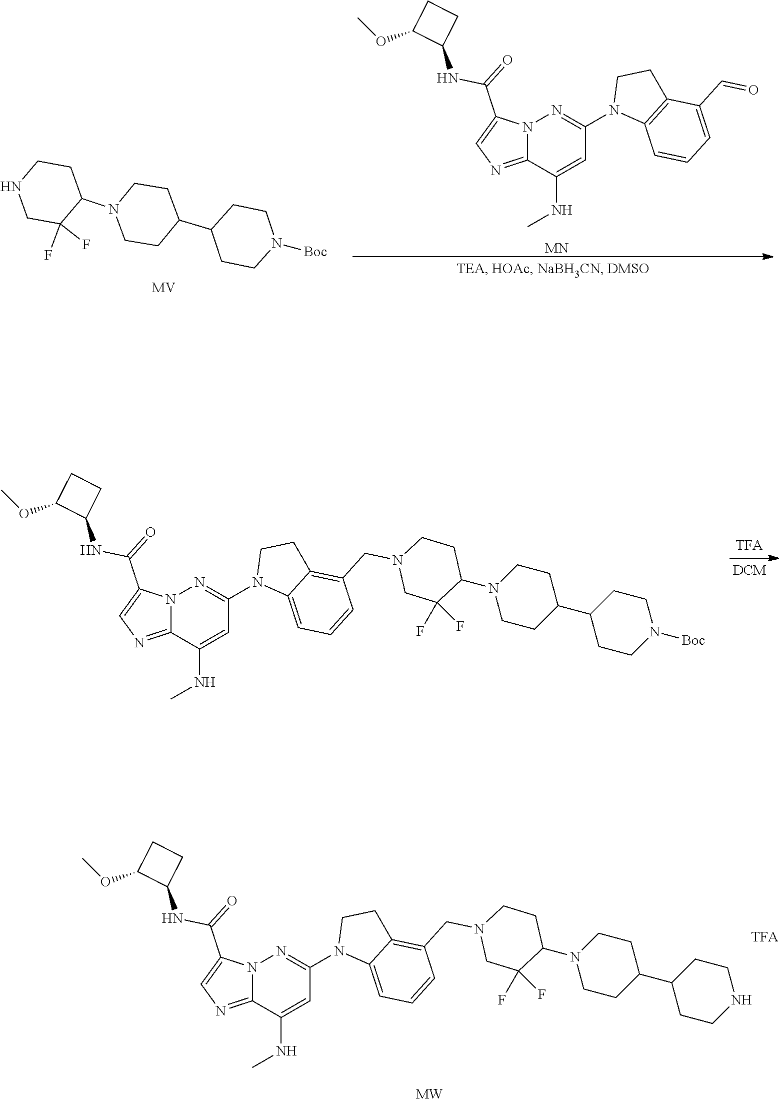

Step 1—Tert-butyl 1′-(3,3-difluoro-1-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}piperidin-4-yl)-[4,4′-bipiperidine]-1-carboxylate. To a stirred solution of tert-butyl 1′-(3,3-difluoropiperidin-4-yl)-[4,4′-bipiperidine]-1-carboxylate (500 mg, 1.29 mmol, Intermediate MV) in DMSO (6 mL) was added TEA (0.54 mL, 3.87 mmol) at rt under nitrogen atmosphere. The resulting mixture was stirred for 5 min at rt under nitrogen atmosphere. To the above mixture was added 6-(4-formyl-2,3-dihydroindol-1-yl)-N-[(1R,2R)-2-methoxycyclobutyl]-8-(methylamino)imidazo[1,2-b]pyridazine-3-carboxamide (542.52 mg, 1.290 mmol, Intermediate MN) and HOAc (0.37 mL, 6.45 mmol) at rt. The resulting mixture was stirred for an additional 30 min at 50° C. The mixture was then cooled to rt and NaBH₃CN (162.16 mg, 2.580 mmol) was added at rt. The resulting mixture was stirred for an additional 2 h at 50° C. On completion, the mixture was cooled to rt and concentrated under reduced pressure. The residue was purified by reverse flash chromatography (column, C18 silica gel; mobile phase A: water (10 mmol/L NH₄HCO₃), mobile phase B: ACN, 40% to 70% gradient in 30 min; detector, UV 254 nm; the fractions were collected at 60%) and concentrated under reduced pressure to afford the title compound (350 mg, 34% yield) as a yellow solid. LC/MS (ESI, m/z): [(M+H)]⁺=792.4

Step 2—6-{4-[(4-{[4,4′-Bipiperidin]-1-yl}-3,3-difluoropiperidin-1-yl)methyl]-2,3-dihydroindol-1-yl}-N-[(1R,2R)-2-methoxycyclobutyl]-8-(methylamino)imidazo[1,2-b]pyridazine-3-carboxamide trifluoroacetate. To a stirred solution of tert-butyl 1′-(3,3-difluoro-1-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}piperidin-4-yl)-[4,4′-bipiperidine]-1-carboxylate (350 mg, 0.442 mmol) in DCM (4 mL) was added TFA (2 mL) at rt under nitrogen atmosphere. The resulting mixture was stirred for 1 h at rt under nitrogen atmosphere. On completion, the mixture was concentrated under reduced pressure. The residue was purified by trituration with ethyl ether (15 mL) to afford the title compound (300 mg, 86% yield) as a yellow solid. LC/MS (ESI, m/z): [(M+H)]⁺=693.1

### 5-Fluoro-6-(6-fluoroindolin-4-yl)nicotinaldehyde (Intermediate MX)

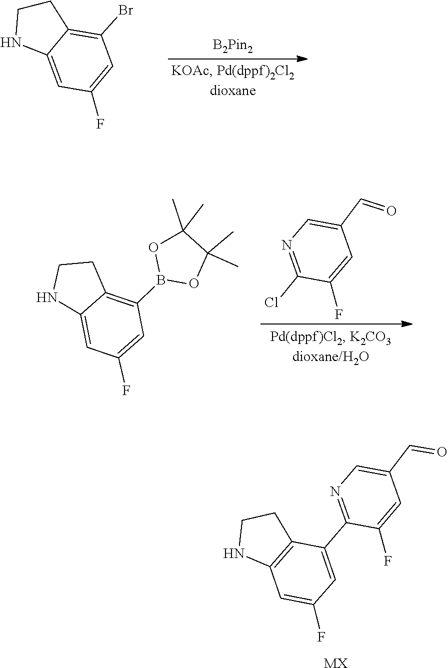

Step 1—6-Fluoro-4-(4,4,5,5-tetramethyl-1,3,2-dioxaborolan-2-yl)indoline. To a stirred solution of 4,4,4′,4′,5,5,5′,5′-octamethyl-2,2′-bi(1,3,2-dioxaborolane) (11.75 g, 46.28 mmol) and 4-bromo-6-fluoroindoline (5.00 g, 23.1 mmol, synthesized via Step 1 of Intermediate MI) in 1,4-dioxane (50 mL) were added KOAc (6.81 g, 69.4 mmol) and Pd(dppf)Cl₂CH₂CH₂Cl₂ (1.89 g, 2.31 mmol) at rt under nitrogen atmosphere. The resulting mixture was stirred for 2 h at 90° C. under nitrogen atmosphere. On completion, the mixture was cooled to rt and concentrated under vacuum. The residue was purified by silica gel column chromatography, eluted with petroleum ether/EtOAc (1:1), to afford the title compound (5 g, 82% yield) as a brown oil. LC/MS (ESI, m/z): [(M+1)]⁺=264.2.

Step 2—5-Fluoro-6-(6-fluoroindolin-4-yl)nicotinaldehyde. To a stirred solution of 6-fluoro-4-(4,4,5,5-tetramethyl-1,3,2-dioxaborolan-2-yl)indoline (5.00 g, 19.0 mmol) and 6-chloro-5-fluoronicotinaldehyde (3.03 g, 19.0 mmol, CAS #950691-52-8) in dioxane (80 mL) and H₂O (16 mL) were added K2CO₃ (7.88 g, 57.0 mmol) and Pd(dppf)C₁₋₂·CH₂Cl₂ (1.55 g, 1.90 mmol) at rt under nitrogen atmosphere. The resulting mixture was stirred for 2 h at 90° C. under nitrogen atmosphere. On completion, the mixture was cooled to rt and purified by silica gel column chromatography, eluted with petroleum ether/EtOAc (1:1), to afford the title compound (4.6 g, 93% yield) as a yellow solid. LC/MS (ESI, m/z): [(M+1)]⁺=261.1.

### 6-(6-Fluoro-4-(3-fluoro-5-formylpyridin-2-yl)indolin-1-yl)-N-((1R,2R)-2-methoxycyclobutyl)-8-(methylamino)imidazo[1,2-b]pyridazine-3-carboxamide trifluoroacetate (Intermediate MY)

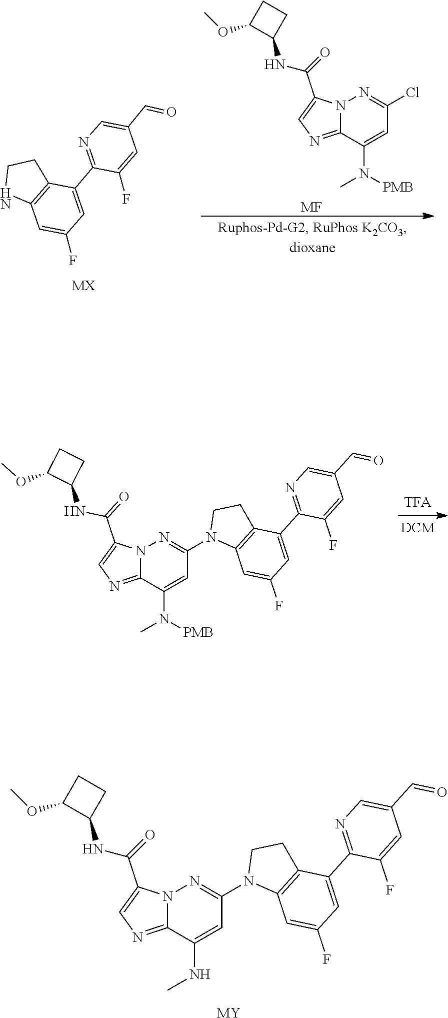

Step 1—6-(6-Fluoro-4-(3-fluoro-5-formylpyridin-2-yl)indolin-1-yl)-8-((4-methoxybenzyl)(methyl)amino)-N-((1R,2R)-2-methoxycyclobutyl)imidazo[1,2-b]pyridazine-3-carboxamide. To a stirred solution of 5-fluoro-6-(6-fluoroindolin-4-yl)nicotinaldehyde (4.58 g, 17.6 mmol, Intermediate MX) and 6-chloro-8-((4-methoxybenzyl)(methyl)amino)-N-((1R,2R)-2-methoxycyclobutyl)imidazo[1,2-b]pyridazine-3-carboxamide (6.30 g, 14.7 mmol, Intermediate MF) in 1,4-dioxane (100 mL) were added K₂CO₃ (6.08 g, 44.0 mmol), RuPhos (1.37 g, 2.93 mmol) and RuPhos-PdCl-2nd G (2.28 g, 2.93 mmol) at rt under nitrogen atmosphere. The resulting mixture was stirred for 2 h at 100° C. under nitrogen atmosphere. On completion, the mixture was cooled to rt and purified by silica gel column chromatography, eluted with petroleum ether/EtOAc, to afford the title compound (4.1 g, 43%) as a yellow solid. LC/MS (ESI, m/z): [(M+1)]⁺=654.4.

Step 2—6-(6-Fluoro-4-(3-fluoro-5-formylpyridin-2-yl)indolin-1-yl)-N-((1R,2R)-2-methoxycyclobutyl)-8-(methylamino)imidazo[1,2-b]pyridazine-3-carboxamide trifluoroacetate. To a stirred solution of 6-(6-fluoro-4-(3-fluoro-5-formylpyridin-2-yl)indolin-1-yl)-8-((4-methoxybenzyl)(methyl)amino)-N-((1R,2R)-2-methoxycyclobutyl)imidazo[1,2-b]pyridazine-3-carboxamide (4.00 g, 6.12 mmol) in DCM (60 mL) was added TFA (20 mL) dropwise at rt. The resulting mixture was stirred for 1 h at rt. On completion, the mixture was concentrated under vacuum. The residue was purified by trituration with ethyl ether (100 mL) and the solid was dried under vacuum to afford the title compound (4.5 g, TFA salt) as a light yellow solid. LC/MS (ESI, m/z): [(M+1)]⁺=534.4.

### 6-(4-(5-((5,5-difluoro-2,7-diazaspiro[3.5]nonan-2-yl)methyl)-3-fluoropyridin-2-yl)-6-fluoroindolin-1-yl)-N-((1R,2R)-2-methoxycyclobutyl)-8-(methylamino)imidazo[1,2-b]pyridazine-3-carboxamide trifluoroacetate (Intermediate MZ)

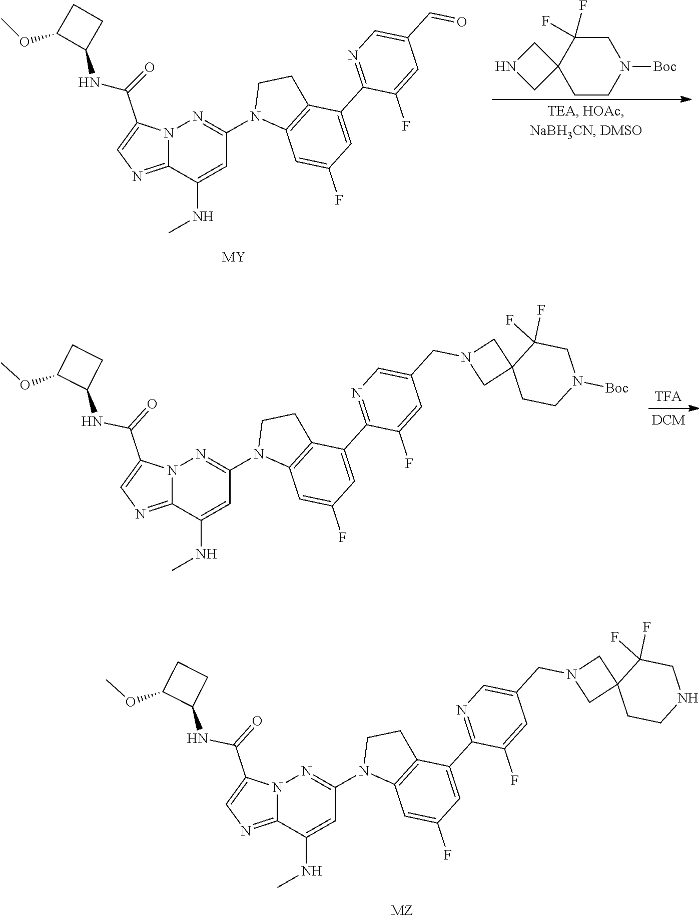

Step 1—Tert-butyl 5,5-difluoro-2-((5-fluoro-6-(6-fluoro-1-(3-(((1R,2R)-2-methoxycyclobutyl)carbamoyl)-8-(methylamino)imidazo[1,2-b]pyridazin-6-yl)indolin-4-yl)pyridin-3-yl)methyl)-2,7-diazaspiro[3.5]nonane-7-carboxylate. To a stirred solution of 6-(6-fluoro-4-(3-fluoro-5-formylpyridin-2-yl)indolin-1-yl)-N-((1R,2R)-2-methoxycyclobutyl)-8-(methylamino)imidazo[1,2-b]pyridazine-3-carboxamide trifluoroacetate (2.50 g, 4.69 mmol, Intermediate MY) in DMSO (20 mL) were added TEA (0.47 g, 4.69 mmol), tert-butyl 5,5-difluoro-2,7-diazaspiro[3.5]nonane-7-carboxylate (1.23 g, 4.69 mmol) and HOAc (0.84 g, 14.06 mmol) at rt under nitrogen atmosphere. The resulting mixture was stirred for 1 h at 50° C. Next, NaBH₃CN (1.18 g, 18.74 mmol) was added in portions at rt. The resulting mixture was stirred overnight at 50° C. under nitrogen atmosphere. On completion, the mixture was cooled to rt and purified by reversed-phase flash chromatography (column, C18 silica gel; mobile phase, MeCN in Water (10 mmol/L NH4HCO₃), 50% to 95% gradient in 30 min; detector, UV 254 nm) to afford the title compound (1.5 g, 41% yield) as a white solid. LC/MS (ESI, m/z): [(M+1)]⁺=780.3.

Step 2—6-(4-(5-((5,5-difluoro-2,7-diazaspiro[3.5]nonan-2-yl)methyl)-3-fluoropyridin-2-yl)-6-fluoroindolin-1-yl)-N-((1R,2R)-2-methoxycyclobutyl)-8-(methylamino)imidazo[1,2-b]pyridazine-3-carboxamide trifluoroacetate. To a stirred solution of tert-butyl 5,5-difluoro-2-((5-fluoro-6-(6-fluoro-1-(3-(((1R,2R)-2-methoxycyclobutyl)carbamoyl)-8-(methylamino)imidazo[1,2-b]pyridazin-6-yl)indolin-4-yl)pyridin-3-yl)methyl)-2,7-diazaspiro[3.5]nonane-7-carboxylate (1.50 g, 1.92 mmol) in DCM (21 mL) was added TFA (7 mL) dropwise at rt and the mixture was stirred for 1 h at rt. On completion, the mixture was concentrated under vacuum. The residue was purified by trituration with ethyl ether (100 mL) and the solid was dried under vacuum to afford the title compound (1.5 g, TFA salt) as a light yellow solid. LC/MS (ESI, m/z): [(M+1)]⁺=680.3.

### 6-(4-(((R)-3,3-difluoro-4-(3,9-diazaspiro[5.5]undecan-3-yl)piperidin-1-yl)methyl)indolin-1-yl)-N-((1R,2R)-2-methoxycyclobutyl)-8-(methylamino)imidazo[1,2-b]pyridazine-3-carboxamide trifluoroacetate (Intermediate NA)

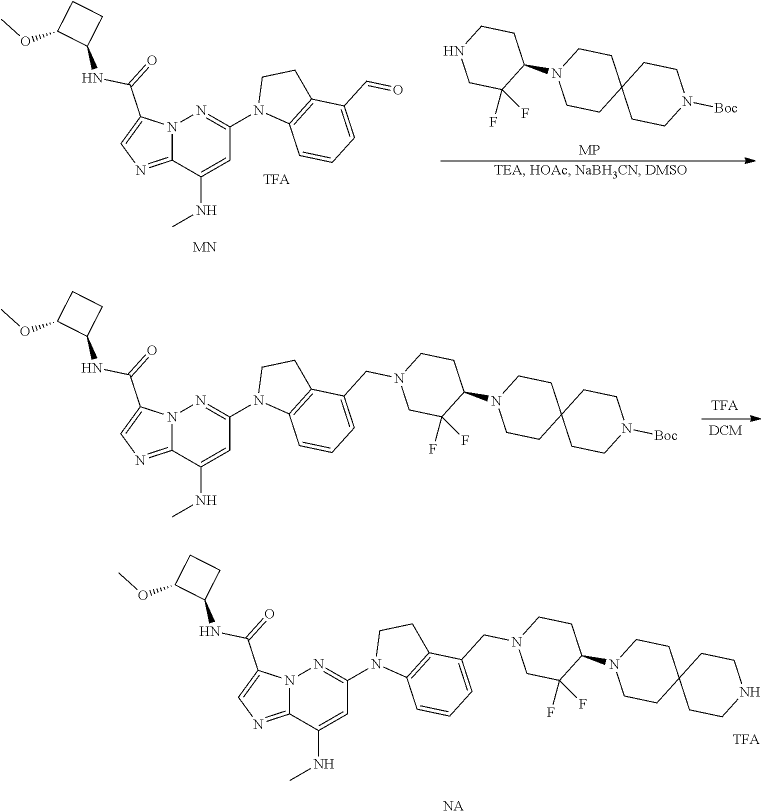

Step 1—Tert-butyl 9-((R)-3,3-difluoro-1-((1-(3-(((1R,2R)-2-methoxycyclobutyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate. To a stirred solution of 6-(4-formylindolin-1-yl)-N-((1R,2R)-2-methoxycyclobutyl)-8-(methylamino)imidazo[1,2-b]pyridazine-3-carboxamide 2,2,2-trifluoroacetate (1.00 g, 1.87 mmol, Intermediate MN) and TEA (94 mg, 0.94 mmol) in DMSO (12 mL) were added tert-butyl (R)-9-(3,3-difluoropiperidin-4-yl)-3,9-diazaspiro[5.5]undecane-3-carboxylate (699 mg, 1.87 mmol, Intermediate MP) and AcOH (449 mg, 7.48 mmol) at rt. The resulting mixture was stirred for 1 h at 50° C. Next, NaBH₃CN (470 mg, 7.484 mmol) was added at rt and the mixture was stirred for additional 1 h at 50° C. On completion, the mixture was cooled to rt and purified by reversed-phase flash chromatography (Column: Spherical C18, 20-40 μm, 120 g; Mobile Phase A: Water (plus 10 mM NH4HCO₃), Mobile Phase B: ACN; Flow rate: 50 mL/min; Gradient (B %): 5%˜40%, 4 min; 40%˜80%, 20 min; 80%˜95%; 2 min; 95%, 5 min; Detector: 254 nm; the fractions containing desired product were collected at 76% B) and concentrated under reduced pressure to afford the title compound (890 mg, 61% yield) as an off-white solid. LC/MS (ESI, m/z): [(M+1)]⁺=778.5.

Step 2—6-(4-(((R)-3,3-difluoro-4-(3,9-diazaspiro[5.5]undecan-3-yl)piperidin-1-yl)methyl)indolin-1-yl)-N-((1R,2R)-2-methoxycyclobutyl)-8-(methylamino)imidazo[1,2-b]pyridazine-3-carboxamide trifluoroacetate. A solution of tert-butyl 9-((R)-3,3-difluoro-1-((1-(3-(((1R,2R)-2-methoxycyclobutyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate (440 mg, 0.566 mmol) in DCM (20 mL) and TFA (4 mL) was stirred for 1 h at room temperature. The resulting mixture was concentrated under reduced pressure. The resulting mixture was diluted with diethyl ether (20 mL). The precipitated solids were collected by filtration and washed with diethyl ether (2×5 mL). This resulted in 6-(4-(((R)-3,3-difluoro-4-(3,9-diazaspiro[5.5]undecan-3-yl)piperidin-1-yl)methyl)indolin-1-yl)-N-((1R,2R)-2-methoxycyclobutyl)-8-(methylamino)imidazo[1,2-b]pyridazine-3-carboxamide trifluoroacetate (530 mg, TFA salt) as a white solid. LC/MS (ESI, m/z): [(M+1)]⁺=678.5.

### Tert-butyl 7-(3,3-difluoropiperidin-4-yl)-2,7-diazaspiro[3.5]nonane-2-carboxylate (Intermediate NB)

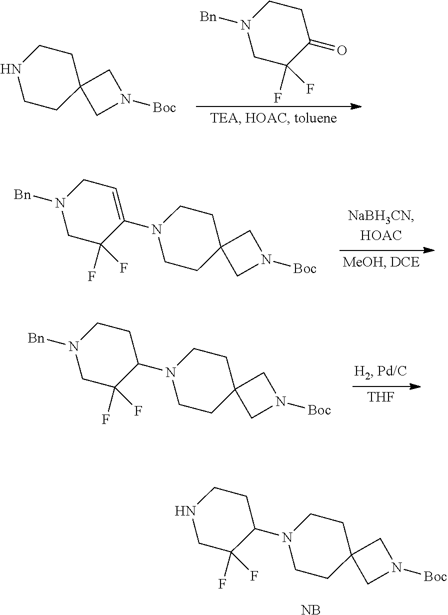

Step 1—Tert-butyl 7-(1-benzyl-3,3-difluoro-2,6-dihydropyridin-4-yl)-2,7-diazaspiro[3.5]nonane-2-carboxylate. To a stirred mixture of tert-butyl 2,7-diazaspiro[3.5]nonane-2-carboxylate (10 g, 40 mmol, CAS #236406-55-6) in toluene (500 mL) was added TEA (9.22 mL, 66.3 mmol) under nitrogen atmosphere, followed by the addition of 1-benzyl-3,3-difluoropiperidin-4-one (19.90 g, 88.37 mmol) at rt. To the above mixture was added HOAc (7.59 mL, 133 mmol) at rt. The resulting mixture was stirred for an additional 16 h at 120° C. On completion, the mixture cooled to rt and diluted with water (200 mL). The resulting mixture was extracted with EtOAc (3×300 mL). The combined organic layers were washed with sat. NaCl (aq.) (1×300 mL), dried over anhydrous Na₂SO₄, filtered, and the filtrate was concentrated under reduced pressure to afford the title compound (25 g) as a yellow oil. LC/MS (ESI, m/z): [(M+H)]⁺=434.2.

Step 2—Tert-butyl 7-(1-benzyl-3,3-difluoropiperidin-4-yl)-2,7-diazaspiro[3.5]nonane-2-carboxylate. To a stirred solution of tert-butyl 7-(1-benzyl-3,3-difluoro-2,6-dihydropyridin-4-yl)-2,7-diazaspiro[3.5]nonane-2-carboxylate (25 g, 58 mmol) in HOAc (10 mL), MeOH (100 mL) and DCE (100 mL) was added NaBH₃CN (18.12 g, 288.3 mmol) at rt. The resulting mixture was stirred for an additional 16 h at rt. On completion, the mixture was concentrated under vacuum. The mixture was diluted with sat. NH₄Cl (aq.) (200 mL) at rt. The resulting mixture was extracted with EtOAc (3×500 mL). The combined organic layers were dried over anhydrous Na₂SO₄, filtered, and the filtrate was concentrated under reduced pressure. The residue was purified by silica gel column chromatography, eluted with PE/EA (2:1), to afford the title compound (14 g, 56% yield) as a yellow solid. LC/MS (ESI, m/z): [(M+H)]⁺=436.3.

Step 3—Tert-butyl 7-(3,3-difluoropiperidin-4-yl)-2,7-diazaspiro[3.5]nonane-2-carboxylate. A solution of tert-butyl 7-(1-benzyl-3,3-difluoropiperidin-4-yl)-2,7-diazaspiro[3.5]nonane-2-carboxylate (800 mg, 1.84 mmol) in THF (10 mL) was added Pd/C (977.32 mg, 9.185 mmol) at rt under nitrogen atmosphere. The resulting mixture was purged with hydrogen three times and stirred for an additional 1 h at rt. On completion, the mixture was filtered, and the filter cake was washed with THF (3×10 mL). The filtrate was concentrated under reduced pressure to afford the title compound (600 mg, 95% yield) as a grey solid. LC/MS (ESI, m/z): [(M+H)]⁺=346.2.

### 6-{4-[(4-{2,7-Diazaspiro[3.5]nonan-7-yl}-3,3-difluoropiperidin-1-yl)methyl]-2,3-dihydroindol-1-yl}-N-[(1R,2R)-2-methoxycyclobutyl]-8-(methylamino)imidazo[1,2-b]pyridazine-3-carboxamide trifluoroacetate (Intermediate NC)

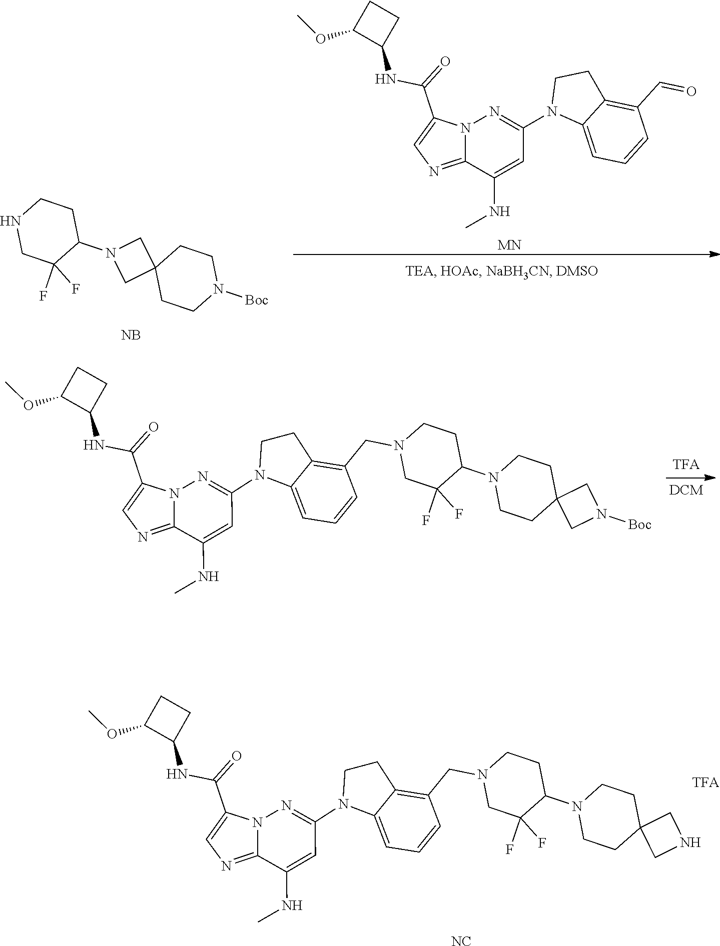

Step 1—Tert-butyl 7-(3,3-difluoro-1-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}piperidin-4-yl)-2,7-diazaspiro[3.5]nonane-2-carboxylate. To a stirred solution of tert-butyl 7-(3,3-difluoropiperidin-4-yl)-2,7-diazaspiro[3.5]nonane-2-carboxylate (600 mg, 1.74 mmol, Intermediate NB) in DMSO (10 mL) was added TEA (0.72 mL, 5.21 mmol) at rt under nitrogen atmosphere. The resulting mixture was stirred for 5 min at rt under nitrogen atmosphere. To the above mixture was added 6-(4-formyl-2,3-dihydroindol-1-yl)-N-[(1R,2R)-2-methoxycyclobutyl]-8-(methylamino)imidazo[1,2-b]pyridazine-3-carboxamide (730.34 mg, 1.737 mmol, Intermediate MN) and HOAc (0.50 mL, 8.69 mmol) at it. The resulting mixture was stirred for an additional 30 min at 50° C. The mixture then was allowed to cool down to room temperature. Next, NaBH₃CN (218.30 mg, 3.474 mmol) was added at rt then the resulting mixture was stirred for an additional 2 h at 50° C. On completion, the mixture was cooled to rt and concentrated under reduced pressure. The residue was purified by reverse flash chromatography (column, C18 silica gel; mobile phase A: water (10 mmol/L NH₄HCO₃), mobile phase B: ACN, 50% to 70% gradient in 30 min; detector, UV 254 nm; the fractions were collected at 55%) and concentrated under reduced pressure to afford the title compound (400 mg, 31% yield) as a yellow oil. LC/MS (ESI, m/z): [(M+H)]⁺=750.4.

Step 2—6-{4-[(4-{2,7-Diazaspiro[3.5]nonan-7-yl}-3,3-difluoropiperidin-1-yl)methyl]-2,3-dihydroindol-1-yl}-N-[(1R,2R)-2-methoxycyclobutyl]-8-(methylamino)imidazo[1,2-b]pyridazine-3-carboxamide trifluoroacetate. To a stirred solution of tert-butyl 7-(3,3-difluoro-1-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}piperidin-4-yl)-2,7-diazaspiro[3.5]nonane-2-carboxylate (400 mg, 0.533 mmol) in DCM (6 mL) was added TFA (3 mL) at rt under nitrogen atmosphere and the mixture was stirred for 1 h at rt under nitrogen atmosphere. On completion, the mixture was concentrated under reduced pressure. The residue was purified by trituration with ethyl ether (15 mL) to afford the title compound (350 mg, 88% yield) as a yellow solid. LC/MS (ESI, m/z): [(M+H)]⁺=650.3.

### Tert-butyl 4-((3′,3′-difluoro-[1,4′-bipiperidin]-4-yl)methyl)piperidine-1-carboxylate (Intermediate ND)

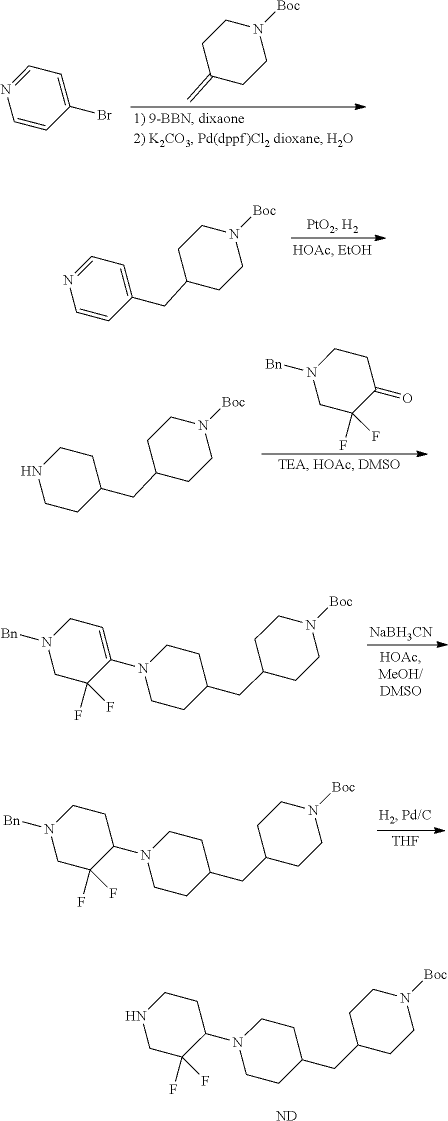

Step 1—Tert-butyl 4-(pyridin-4-ylmethyl) piperidine-1-carboxylate. To a stirred mixture of 4-bromopyridine (8.81 g, 55.8 mmol) in dioxane (100 mL) was added 9-borabicyclo[3.3.1]nonane (6.80 g, 55.8 mmol) at rt. The resulting mixture was stirred for 1 h at 50° C. To the above mixture was added tert-butyl 4-methylidenepiperidine-1-carboxylate (10 g, 50 mmol) and H₂O (10 mL) at rt. The resulting mixture was stirred for an additional 1 h at 90° C. On completion, the mixture was extracted with EtOEt (3×20 mL). The combined organic layers were washed with EtOAc (3×10 mL), dried over anhydrous Na₂SO₄, filtered, and the filtrate was concentrated under reduced pressure. The residue was purified by silica gel column chromatography, eluted with PE/EA (5:1), to afford the title compound (16 g, 97% yield) as a yellow oil. LC/MS (ESI, m/z): [(M+1)]⁺=277.4.

Step 2—Tert-butyl 4-(piperidin-4-ylmethyl)piperidine-1-carboxylate. To a solution of tert-butyl 4-(pyridin-4-ylmethyl) piperidine-1-carboxylate (16 g, 58 mmol) in EtOH (50 mL) was added PtO₂ (13.15 g, 57.89 mmol) under nitrogen atmosphere. The reaction system was degassed under vacuum and purged with H₂ several times, then was hydrogenated under H2 balloon (˜1 atm) at 25° C., for 3 h. After completion of the reaction, Pd/C was filtered off through celite and the filter cake was washed with EtOH (50 mL), and the filtrate was concentrated under reduced pressure. The residue was purified by reverse phase flash chromatography (Column: WelFlash™ C18-I, 20-40 μm, 330 g; Eluent A: Water (plus 10 mmol/L NH4HCO₃); Eluent B: ACN; Gradient: 25%-40% B in 25 min; Flow rate: 80 mL/min; Detector: 220/254 nm; desired fractions were collected at 30% B) and concentrated under reduced pressure to afford the title compound (6 g, 37% yield) as a yellow solid. LC/MS (ESI, m/z): [(M+1)]⁺=283.3

Step 3 and Step 4—Tert-butyl 4-((1-(1-benzyl-3,3-difluoro-1,2,3,6-tetrahydropyridin-4-yl)piperidin-4-yl)methyl) piperidine-1-carboxylate. To a stirred mixture of tert-butyl 4-(piperidin-4-ylmethyl)piperidine-1-carboxylate (4 g, 14 mmol) and TEA (4.30 g, 42.5 mmol) in DMSO (20 mL) were added 1-benzyl-3,3-difluoropiperidin-4-one (3.19 g, 14.2 mmol) and AcOH (5.10 g, 85.0 mmol) at rt. The resulting mixture was stirred for 2 h at 100° C. Next, NaBH₃CN (5.34 g, 84.978 mmol) was added at rt and the resulting mixture was stirred for an additional 16 h at 50° C. On completion, the residue was purified directly by reverse phase flash chromatography (Column: WelFlash™ C18-I, 20-40 μm, 330 g; Eluent A: Water (plus 10 mmol/L NH4HCO₃); Eluent B: ACN; Gradient: 90%-98% B in 25 min; Flow rate: 80 mL/min; Detector: 254 nm; desired fractions were collected at 96% B and concentrated under reduced pressure to afford the title compound (3.3 g, 47% yield) as a white solid. LC/MS (ESI, m/z): [(M+1)]+=492.3.

Step 5—Tert-butyl 4-((3′,3′-difluoro-[1,4′-bipiperidin]-4-yl)methyl)piperidine-1-carboxylate. To a stirred solution of tert-butyl 4-((1-(1-benzyl-3,3-difluoro-1,2,3,6-tetrahydropyridin-4-yl)piperidin-4-yl)methyl) piperidine-1-carboxylate (1.5 g, 3.1 mmol) in THF (15 mL) and MeOH (5 mL) were added Pd/C (500 mg, 10 wt %) at rt under nitrogen atmosphere. The resulting mixture was stirred for 1 h at rt under hydrogen atmosphere. The resulting mixture was filtered, the filter cake was washed with MeOH (5 mL) (3×30 mL), and the filtrate was concentrated under reduced pressure to give the title compound (1.35 g, 89% yield) as a grey solid. LC/MS (ESI, m/z): [(M+1)]⁺=402.3.

### 6-(4-((3′,3′-Difluoro-4-(piperidin-4-ylmethyl)-[1,4′-bipiperidin]-1′-yl)methyl)indolin-1-yl)-N-((1R,2R)-2-methoxycyclobutyl)-8-(methylamino)imidazo[1,2-b]pyridazine-3-carboxamide trifluoroacetate (Intermediate NE)

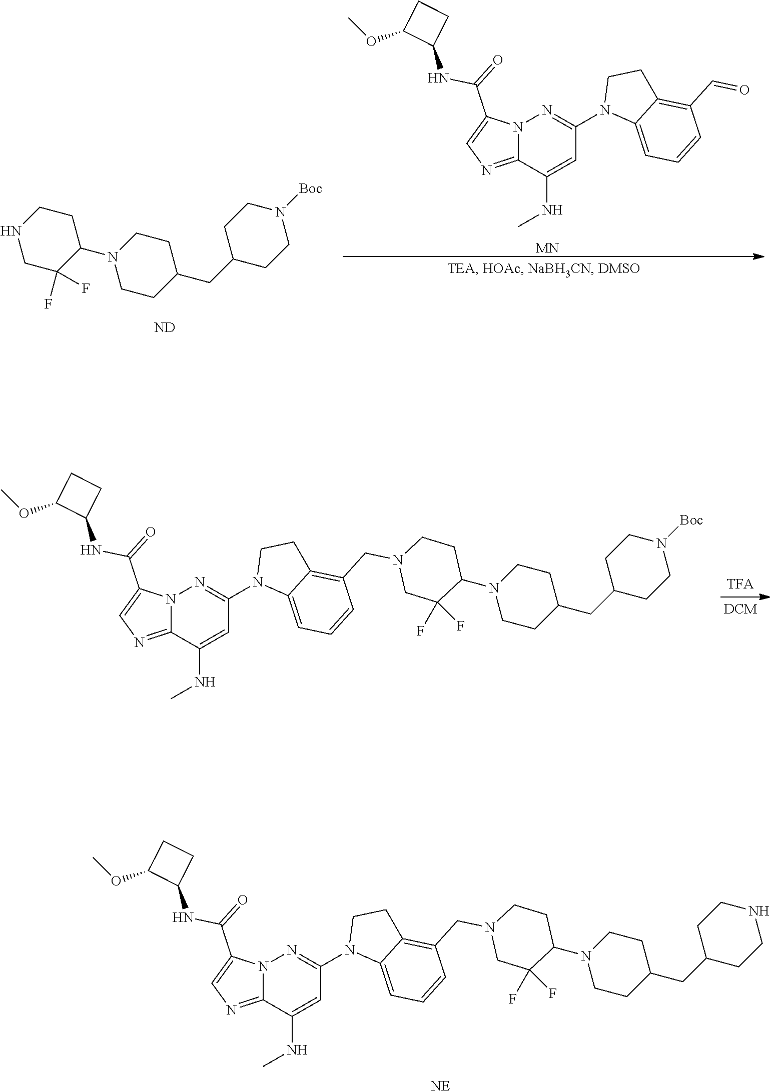

Step 1—Tert-butyl 4-((3′,3′-difluoro-1′-((1-(3-(((1R,2R)-2-methoxycyclobutyl)carbamoyl)-8-(methylamino)imidazo[1,2-b]pyridazin-6-yl)indolin-4-yl)methyl)-[1,4′-bipiperidin]-4-yl)methyl) piperidine-1-carboxylate To a stirred solution of tert-butyl 4-((3′,3′-difluoro-[1,4′-bipiperidin]-4-yl)methyl)piperidine-1-carboxylate (572.99 mg, 1.426 mmol, Intermediate ND) and TEA (288.80 mg, 2.853 mmol) in DMSO (10 mL) was added 6-(4-formylindolin-1-yl)-N-((1R,2R)-2-methoxycyclobutyl)-8-(methylamino)imidazo[1,2-b]pyridazine-3-carboxamide (400 mg, 0.951 mmol, Intermediate MN) and AcOH (171.38 mg, 2.853 mmol) at rt. The resulting mixture was stirred for 1 h at 50° C. under nitrogen atmosphere. To the above mixture was added NaBH₃CN (239.12 mg, 3.804 mmol) at rt and the resulting mixture was stirred for an additional 2 h at 50° C. The mixture product was purified by reverse phase flash chromatography (WelFlash™ C18-I, 20-40 μm, 330 g; Eluent A: Water (plus 10 mmol/L NH₄HCO₃); Eluent B: ACN; Gradient: 25%-55% B in 25 min; Flow rate: 80 mL/min; Detector: 220/254 nm; desired fractions were collected at 50% B) and concentrated under reduced pressure to afford the title compound (600 mg, 75% yield) as a white solid. LC/MS (ESI, m/z): [(M+1)]⁺=806.4.

Step 2—6-(4-((3′,3′-Difluoro-4-(piperidin-4-ylmethyl)-[1,4′-bipiperidin]-1′-yl)methyl)indolin-1-yl)-N-((1R,2R)-2-methoxycyclobutyl)-8-(methylamino)imidazo[1,2-b]pyridazine-3-carboxamide trifluoroacetate. To a stirred solution of tert-butyl 4-((3′,3′-difluoro-1′-((1-(3-(((1R,2R)-2-methoxycyclobutyl)carbamoyl)-8-(methylamino)imidazo[1,2-b]pyridazin-6-yl)indolin-4-yl)methyl)-[1,4′-bipiperidin]-4-yl)methyl)piperidine-1-carboxylate (200 mg, 0.5 mmol) in DCM (9 mL) was added TFA (3 mL) at rt and the mixture was stirred for 1 h at rt under nitrogen atmosphere. The residue was purified by trituration with ethyl ether (30 mL) to give the title compound (170 mg, 95% yield) as a white solid. LC/MS (ESI, m/z): [(M+1)]⁺=706.4.

### 1-(2-Chloro-5-{3,9-diazaspiro[5.5]undecane-3-carbonyl}phenyl)-1,3-diazinane-2,4-dione trifluoroacetate (Intermediate NF)

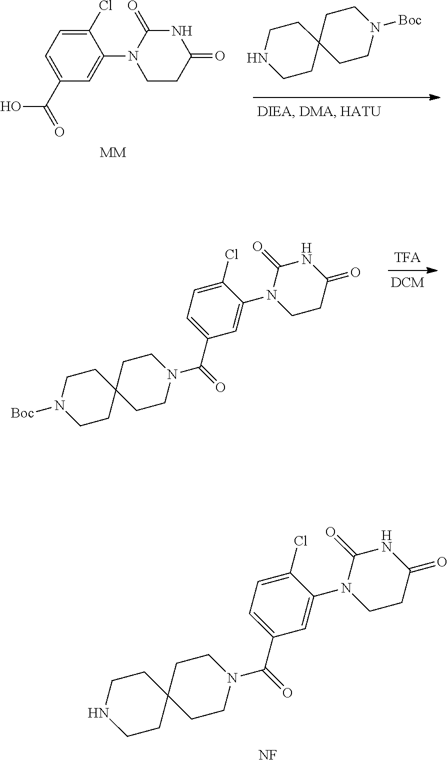

Step 1—Tert-butyl 9-[4-chloro-3-(2,4-dioxo-1,3-diazinan-1-yl)benzoyl]-3,9-diazaspiro[5.5]undecane-3-carboxylate. To a stirred mixture of 4-chloro-3-(2,4-dioxo-1,3-diazinan-1-yl)benzoic acid (500 mg, 1.86 mmol, Intermediate MM) and tert-butyl 3,9-diazaspiro[5.5]undecane-3-carboxylate (473.43 mg, 1.861 mmol) in DMA (6 mL) were added HATU (1061.52 mg, 2.792 mmol) and DIEA (0.996 mL, 5.583 mmol) at rt under nitrogen atmosphere. The resulting mixture was stirred for 2 h at rt under nitrogen atmosphere. On completion, the mixture was concentrated under reduced pressure. The residue was purified by reverse flash chromatography (column, C18 silica gel; mobile phase A: water (10 mmol/L NH₄HCO₃), mobile phase B: ACN, 40% to 70% gradient in 30 min; detector, UV 254 nm; the fractions were collected at 55%) and concentrated under reduced pressure to afford the title compound (800 mg, 85% yield) as a white solid. LC/MS (ESI, m/z): [(M+H)]⁺=505.1.

Step 2—1-(2-Chloro-5-{3,9-diazaspiro[5.5]undecane-3-carbonyl}phenyl)-1,3-diazinane-2,4-dione trifluoroacetate. To a stirred solution of tert-butyl 9-[4-chloro-3-(2,4-dioxo-1,3-diazinan-1-yl)benzoyl]-3,9-diazaspiro[5.5]undecane-3-carboxylate (800 mg, 1.58 mmol) in DCM (6 mL) was added TFA (3 mL) at rt under nitrogen atmosphere and the mixture was stirred for 1 h at rt under nitrogen atmosphere. On completion, the mixture was concentrated under reduced pressure. The residue was purified by trituration with ethyl ether (20 mL) to afford the title compound (790 mg, 99% yield) as a white solid. LC/MS (ESI, m/z): [(M+H)]⁺=405.1.

### N-(3-{5-formyl-6′-[(3S)-3-methoxyoxolan-3-yl]-[2,4′-bipyridin]-2′-yl}-1-methylpyrrolo[2,3-c]pyridin-5-yl)acetamide (Intermediate NG)

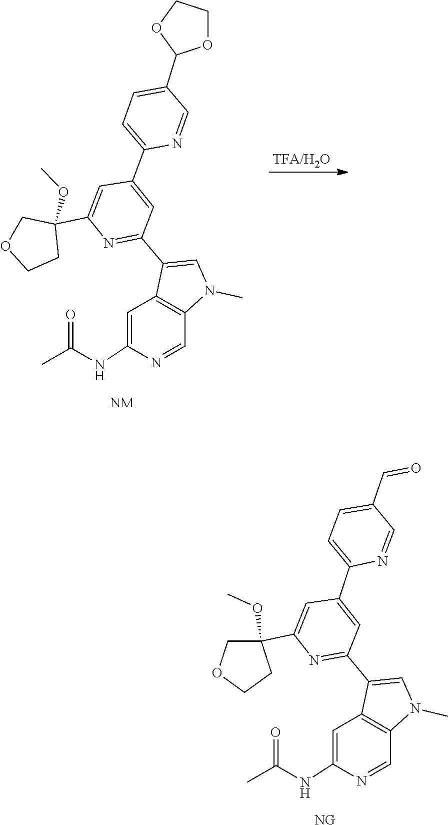

Step 1—N-(3-{5-formyl-6′-[(3S)-3-methoxyoxolan-3-yl]-[2,4′-bipyridin]-2′-yl}-1-methylpyrrolo[2,3-c]pyridin-5-yl)acetamide. To a stirred mixture of N-{3-[5-(1,3-dioxolan-2-yl)-6′-[(3R)-3-methoxyoxolan-3-yl]-[2,4′-bipyridin]-2′-yl]-1-methylpyrrolo[2,3-c]pyridin-5-yl}acetamide (87 mg, 0,169 mmol, Intermediate NM) in H₂O (1 mL) was added TFA (1 mL) at rt. Then the resulting mixture was stirred for 1 h at 50° C. On completion, the mixture was cooled to rt and basified to pH 8 with TEA. The resulting mixture was concentrated under reduced pressure to give the title compound (200 mg) as a brown oil. LC/MS (ESI, m/z): [(M+H)]⁺=472.3.

### 5-(2,4-Dioxotetrahydropyrimidin-1(2H)-yl)-2-fluoro-4-methylbenzoic acid (Intermediate NH)

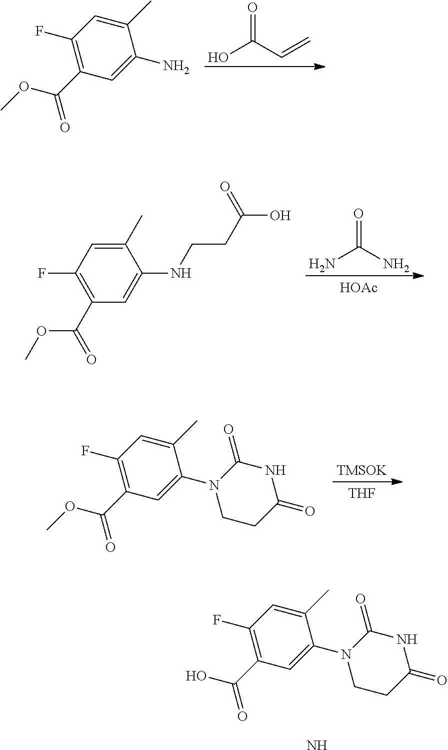

Step 1—3-((4-Fluoro-5-(methoxycarbonyl)-2-methylphenyl)amino)propanoic acid. To a stirred solution of methyl 5-amino-2-fluoro-4-methylbenzoate (2.00 g, 10.9 mmol, CAS #1504965-88-1) in acrylic acid (1.57 g, 21.8 mmol) at rt. The resulting mixture was stirred for 2 h at 100° C. On completion, the mixture was cooled to rt and extracted with EtOAc (3×50 mL). The combined organic layers were washed with water (3×100 mL), and dried over anhydrous Na₂SO₄. After filtration, the filtrate was concentrated under reduced pressure to afford the title compound (3.50 g). LC/MS (ESI, m/z): [(M+1)]⁺=256.1.

Step 2—Methyl 5-(2,4-dioxotetrahydropyrimidin-1(2H)-yl)-2-fluoro-4-methylbenzoate. To a stirred solution of 3-((4-fluoro-5-(methoxycarbonyl)-2-methylphenyl)amino)propanoic acid (3.50 g, 13.7 mmol) in HOAc (5 mL) was added urea (0.82 g, 13.7 mmol) at rt. The resulting mixture was stirred for 16 h at 110° C. On completion, the mixture was cooled to rt and concentrated under reduced pressure. The residue was purified by reverse phase flash chromatography (Column: WelFlash™ C18-I, 20-40 m, 120 g; Eluent A: Water (plus 10 mmol/L FA); Eluent B: ACN; Gradient: 25%-55% B in 25 min; Flow rate: 80 mL/min; Detector: 220/254 nm; desired fractions were collected at 49% B) and concentrated under reduced pressure to afford the title compound (2 g, 52% yield) as a yellow oil. LC/MS (ESI, m/z): [(M+1)]+=281.1.

Step 3—5-(2,4-Dioxotetrahydropyrimidin-1(2H)-yl)-2-fluoro-4-methylbenzoic acid. To a stirred solution of methyl 5-(2,4-dioxotetrahydropyrimidin-1(2H)-yl)-2-fluoro-4-methylbenzoate (0.50 g, 1.78 mmol) in THF (20 mL) was added potassium trimethylsilanolate (1.14 g, 8.92 mmol) at rt and the mixture was stirred for 1 h at rt. On completion, the mixture was acidified to pH 5 with con. HCl. The residue was purified by reverse phase flash chromatography (Column: WelFlash™ C18-I, 20-40 μm, 120 g; Eluent A: Water; Eluent B: ACN; Gradient: 10%-35% B in 25 min; Flow rate: 80 mL/min; Detector: 220/254 nm; desired fractions were collected at 20% B) and concentrated under reduced pressure to afford the title compound (0.30 g, 63% yield) as a white solid. LC/MS (ESI, m/z): [(M+1)]⁺=267.1. ¹H NMR (400 MHz, DMSO-d₆) δ 10.36 (s, 1H), 7.59 (d, J=7.2 Hz, 1H), 7.10 (d, J=11.2 Hz, 1H), 3.84-3.72 (m, 1H), 3.56-3.43 (m, 111), 2.85-2.61 (m, 2H), 2.18 (s, 3H).

### Tert-butyl 2-((6-(indolin-4-yl)pyridin-3-yl)methyl)-7-azaspiro[3.5]nonane-7-carboxylate (Intermediate NI)

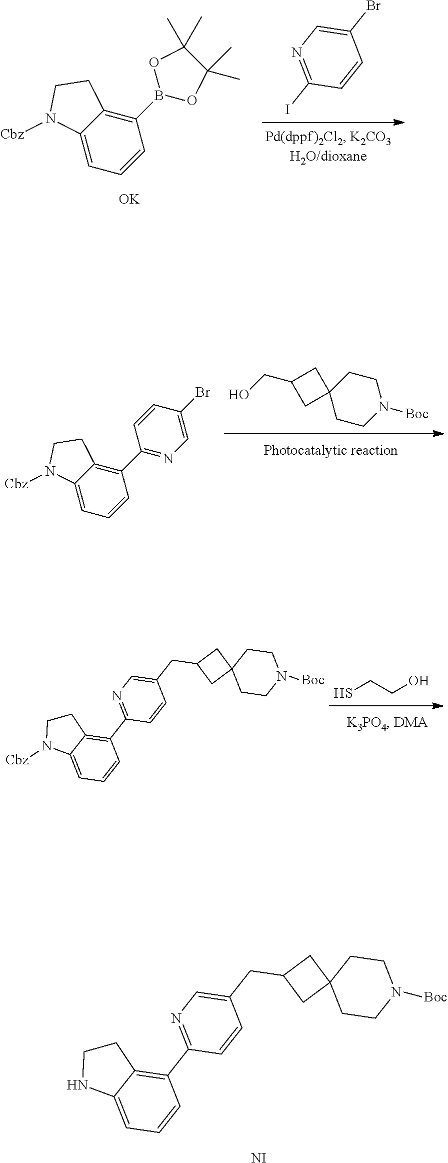

Step 1—Benzyl 4-(5-bromopyridin-2-yl)indoline-1-carboxylate. To a stirred mixture of benzyl 4-(4,4,5,5-tetramethyl-1,3,2-dioxaborolan-2-yl)indoline-1-carboxylate (6.00 g, 15.8 mmol, Intermediate OK) and 5-bromo-2-iodopyridine (4.49 g, 15.8 mmol) in dioxane (100 mL) and H₂O (20 mL) were added K₂CO₃ (6.56 g, 47.5 mmol) and Pd(dppf)Cl₂·CH₂Cl₂ (1.29 g, 1.58 mmol) at rt under nitrogen atmosphere. The resulting mixture was stirred for 1 h at 80° C. under nitrogen atmosphere. On completion, the mixture was cooled to rt and concentrated under reduced pressure. The resulting mixture was extracted with EtOAc (3×100 mL). The combined organic layers were washed with brine (2×200 mL), dried over anhydrous Na₂SO₄, filtered, and the filtrate was concentrated under reduced pressure. The residue was purified by silica gel column chromatography, eluted with petroleum ether/EtOAc (10:1), to afford the title compound (1.7 g, 26% yield) as a brown solid. LC/MS (ESI, m/z): [(M+H)]⁺=409.1, 411.1.

Step 2—Tert-butyl 2-((6-(1-((benzyloxy)carbonyl)indolin-4-yl)pyridin-3-yl)methyl)-7-azaspiro[3.5]nonane-7-carboxylate. An oven-dried 40 mL vial was charged with tert-butyl 2-(hydroxymethyl)-7-azaspiro[3.5]nonane-7-carboxylate (1.50 g, 5.86 mmol, CAS #1356476-27-1), deoxazole (2.32 g, 5.86 mmol, CAS #1207294-92-5) and an X-shaped magnetic stir bar. After the vial was vacuumed and refilled with nitrogen gas twice, t-BuOMe (20 mL) was added and the reaction stirred at rt for 5 min. Then, a pyridine solution (309 mg, 3.1 mmol in 2 mL t-BuOMe) was added dropwise at rt within 2 min. The resulting solution stirred at rt for 10 min. A white solid precipitated out during this time. Another oven-dried glass flask was charged with Ir[ppy]₂(dtbbpy)PF₆ (219 mg, 0.195 mmol), NiBr₂(dtbbpy) (95 mg, 0.195 mmol), quinuclidine (761 mg, 6.84 mmol), isoindoline-1,3-dione (115 mg, 0.782 mmol), benzyl 4-(5-bromopyridin-2-yl)indoline-1-carboxylate (1.60 g, 3.91 mmol) and an X-shape magnetic stir bar. DMA (10 mL) was added to this vial under an atmosphere of nitrogen. The t-BuOMe suspension was injected through the syringe filter into the DMA solution. The resulting reaction mixture was sparged with nitrogen for 15 minutes, then irradiated under 450 nm LED for 16 hrs. After competition of reaction, the reaction mixture was concentrated under reduced pressure. The residue was purified by reverse phase flash chromatography (Column: WelFlash™ C18-I, 20-40 μm, 120 g; Eluent A: Water (plus 10 mmol/L NH4HCO₃); Eluent B: ACN; Gradient: 25%-55% B in 25 min; Flow rate: 80 mL/min; Detector: 220 nm; desired fractions were collected at 50% B) and concentrated under reduced pressure to afford the title compound (1.5 g, 67% yield) as a light yellow solid. LC/MS (ESI, m/z): [(M+H)]⁺=568.3.

Step 3—Tert-butyl 2-((6-(indolin-4-yl)pyridin-3-yl)methyl)-7-azaspiro[3.5]nonane-7-carboxylate. A solution of tert-butyl 2-((6-(1-((benzyloxy)carbonyl)indolin-4-yl)pyridin-3-yl)methyl)-7-azaspiro[3.5]nonane-7-carboxylate (1.50 g, 2.64 mmol), 2-mercaptoethan-1-ol (413 mg, 5.28 mmol) and K₃PO₄ (1.68 g, 7.93 mmol) in DMA (12 mL) was stirred for 16 h at 75° C. After completion of the reaction, the reaction mixture was cooled to rt and purified directly without any workup by reverse phase flash chromatography (Column: Spherical C18, 20-40 m, 120 g; Mobile Phase A: Water (plus 10 mM NH₄HCO₃), Mobile Phase B: ACN; Flow rate: 50 mL/min; Gradient (B %): 5%˜50%, 4 min; 50%˜85%, 20 min; 85%˜95%; 2 min; 95%, 5 min; Detector: 254 nm; the fractions containing desired product were collected at 80% B) and concentrated under reduced pressure to afford the title compound (980 mg, 85% yield) as a yellow solid. LC/MS (ESI, m/z): [(M+H)]⁺=434.3.

### 6-(4-(5-((7-Azaspiro[3.5]nonan-2-yl)methyl)pyridin-2-yl)indolin-1-yl)-N-((1R,2R)-2-methoxycyclobutyl)-8-(methylamino)imidazo[1,2-b]pyridazine-3-carboxamide trifluoroacetate (Intermediate NJ)

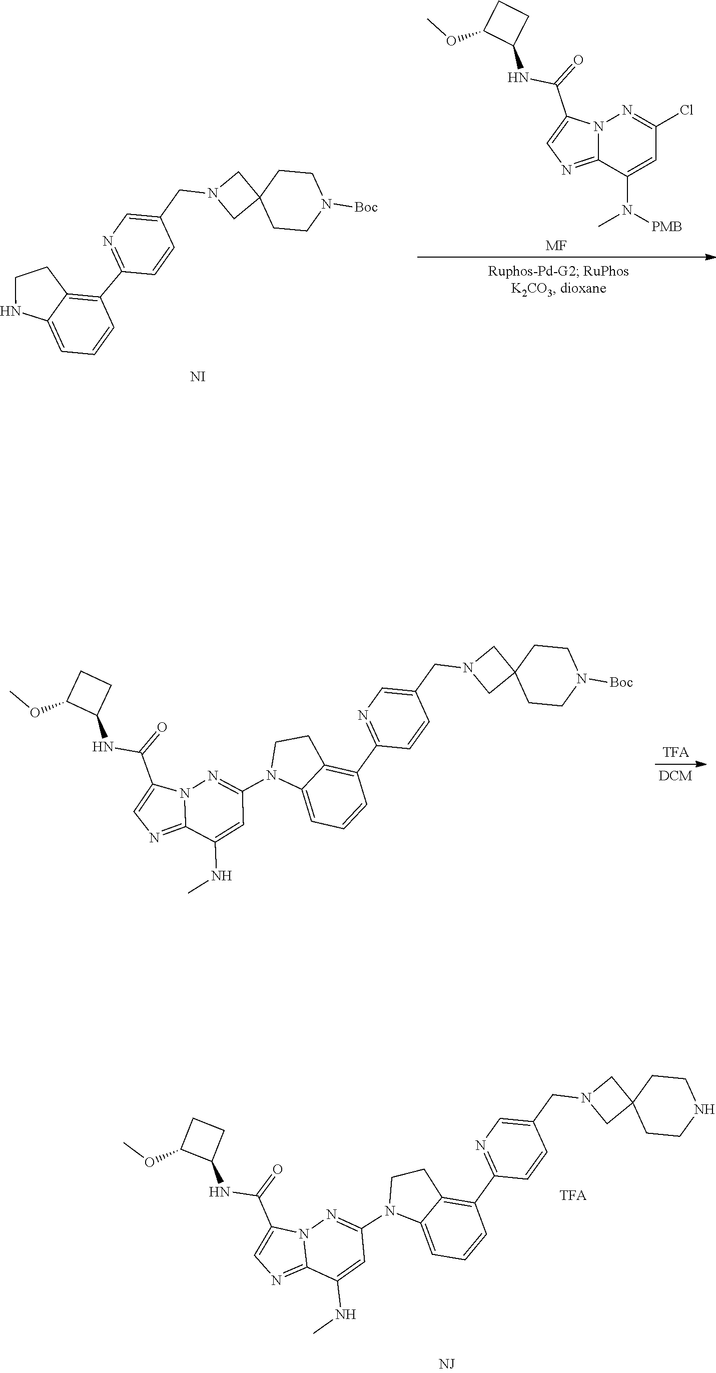

Step 1—Tert-butyl 2-((6-(1-(3-(((1R,2R)-2-methoxycyclobutyl)carbamoyl)-8-(methylamino)imidazo[1,2-b]pyridazin-6-yl)indolin-4-yl)pyridin-3-yl)methyl)-7-azaspiro[3.5]nonane-7-carboxylate. To a stirred mixture of tert-butyl 2-((6-(indolin-4-yl)pyridin-3-yl)methyl)-7-azaspiro[3.5]nonane-7-carboxylate (780 mg, 1.80 mmol, Intermediate NI) and 6-chloro-8-((4-methoxybenzyl)(methyl)amino)-N-((1R,2R)-2-methoxycyclobutyl)imidazo[1,2-b]pyridazine-3-carboxamide (773 mg, 1.80 mmol, Intermediate MF) in dioxane (10 mL) were added K₂CO₃ (746 mg, 5.40 mmol), RuPhos-PdCl-2nd G (140 mg, 0.180 mmol) and RuPhos (168 mg, 0.360 mmol) at rt. The resulting mixture was stirred for 1 h at 100° C. under nitrogen atmosphere. After completion of the reaction, the reaction mixture was cooled to rt and purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 75%-98% B in 25 min; Flow rate: 80 mL/min; Detector: 220 nm; desired fractions were collected at 95% B) and concentrated under reduced pressure to afford the title compound (1 g, 84% yield) as a brown solid. LC/MS (ESI, m/z): [(M+H)]⁺=707.3.

Step 2—6-(4-(5-((7-Azaspiro[3.5]nonan-2-yl)methyl)pyridin-2-yl)indolin-1-yl)-N-((1R,2R)-2-methoxycyclobutyl)-8-(methylamino)imidazo[1,2-b]pyridazine-3-carboxamide trifluoroacetate. To a stirred mixture of tert-butyl 2-((6-(1-(3-(((1R,2R)-2-methoxycyclobutyl)carbamoyl)-8-(methylamino)imidazo[1,2-b]pyridazin-6-yl)indolin-4-yl)pyridin-3-yl)methyl)-7-azaspiro[3.5]nonane-7-carboxylate (1.00 g, 1.41 mmol) in DCM (12 mL) was added TFA (4 mL) at rt and the mixture was stirred for 1 h at rt. On completion, the mixture was purified by trituration with diethyl ether (20 mL) to give the title compound (1.3 g, TFA salt) as a white solid. LC/MS (ESI, m/z): [(M+H)]⁺=607.3.

### 3-(5-{4-[(4S)-3,3-difluoropiperidin-4-yl]piperazin-1-yl}-3-methyl-2-oxo-1,3-benzodiazol-1-yl)piperidine-2,6-dione trifluoroacetate (Intermediate NK)

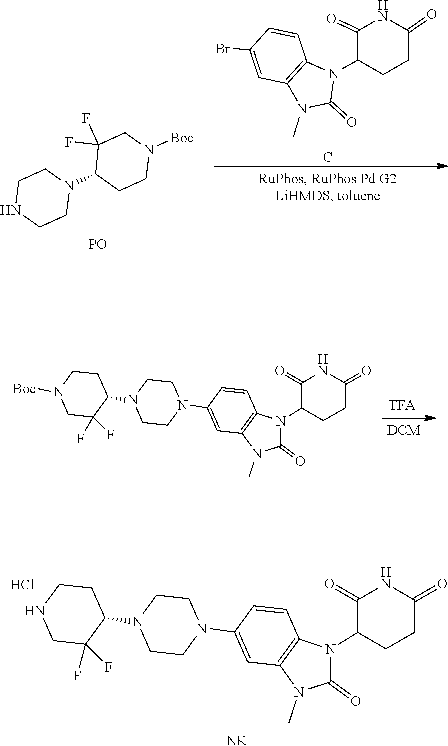

Step 1—Tert-butyl (4S)-4-(4-(1-(2,6-dioxopiperidin-3-yl)-3-methyl-2-oxo-2,3-dihydro-1H-benzo[d]imidazol-5-yl)piperazin-1-yl)-3,3-difluoropiperidine-1-carboxylate. To a stirred mixture of 3-(5-bromo-3-methyl-2-oxo-1,3-benzodiazol-1-yl)piperidine-2,6-dione (10 g, 30 mmol, Intermediate C) and tert-butyl (4R)-3,3-difluoro-4-(piperazin-1-yl)piperidine-1-carboxylate (9.03 g, 29.6 mmol, Intermediate PO) in toluene (140 mL) were added RuPhos (1.38 g, 2.96 mmol) and RuPhos-PdCl-2nd G (2.30 g, 2.96 mmol) at rt. To the above mixture was added LiHMDS (133.07 mL, 133.074 mmol) and the mixture was stirred for 1 h at 80° C. On completion, the mixture was cooled to rt and acidified to pH 4 with FA. The resulting mixture was filtered, the filter cake was washed with ethyl acetate (3×100 mL), and the filtrate was concentrated under reduced pressure. The residue was purified by reverse phase flash chromatography (WelFlash™ C18-I, 20-40 μm, 330 g; Eluent A: Water (plus 10 mmol/L NH₄HCO₃); Eluent B: ACN; Gradient: 35%-55% B in 25 min; Flow rate: 80 mL/min; Detector: 220/254 nm; desired fractions were collected at 49% B) and concentrated under reduced pressure to afford the title compound (8.1 g, 48% yield) as a brown solid. LC/MS (ESI, m/z): [(M+1)]⁺=563.3.

Step 2—3-(5-(4-((S)-3,3-difluoropiperidin-4-yl)piperazin-1-yl)-3-methyl-2-oxo-2,3-dihydro-1H-benzo[d]imidazol-1-yl)piperidine-2,6-dione trifluoroacetate. To a solution of tert-butyl (4R)-4-{4-[1-(2,6-dioxopiperidin-3-yl)-3-methyl-2-oxo-1,3-benzodiazol-5-yl]piperazin-1-yl}-3,3-difluoropiperidine-1-carboxylate (4.1 g, 7.3 mmol) in DCM (45 mL) was added TFA (15 mL) dropwise at rt and the mixture was stirred for 2 h at rt under nitrogen atmosphere. On completion, the mixture was concentrated under reduced pressure. The residue was purified by trituration with ethyl acetate (30 mL). The precipitated solids were collected by filtration and washed with DCM (3×10 mL) to afford the title compound (3.3 g, 75% yield) as a brown yellow solid. LC/MS (ESI, m/z): [(M+1)]⁺=463.2.

### Tert-butyl 5-acetamido-3-(4,4,5,5-tetramethyl-1,3,2-dioxaborolan-2-yl)pyrrolo[2,3-c]pyridine-1-carboxylate (Intermediate NL)

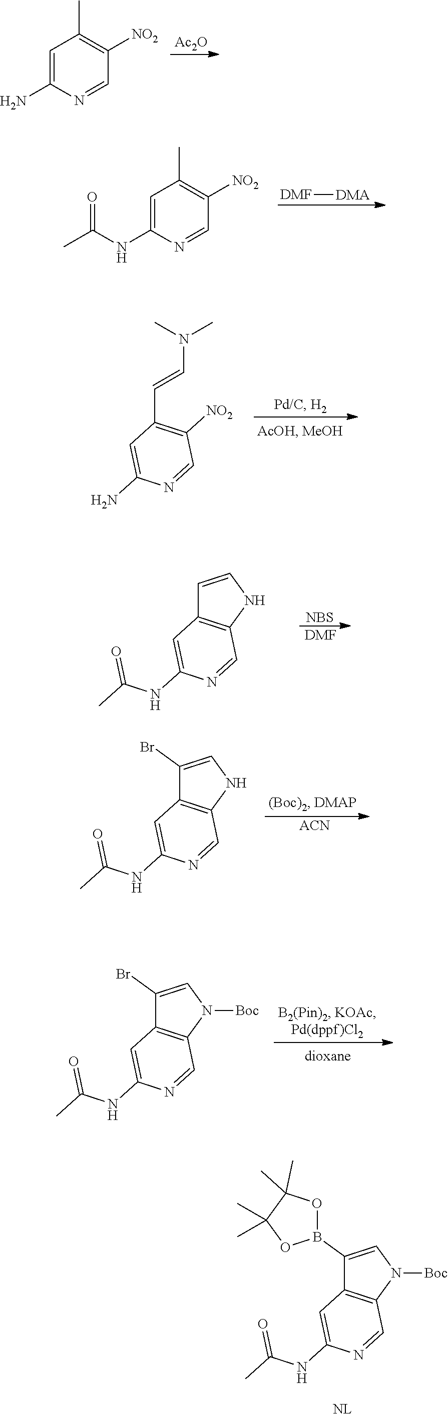

Step 1—N-(4-methyl-5-nitropyridin-2-yl)acetamide. To a stirred solution of 4-methyl-5-nitropyridin-2-amine (5 g, 30 mmol) in Ac₂O (30 mL) under nitrogen atmosphere was stirred for 4 h at 90° C. On completion, the mixture was cooled to rt and quenched with water (100 mL) at rt. The resulting mixture was extracted with EtOAc (3×100 mL). The combined organic layers were washed with EtOAc (3×50 mL), dried over anhydrous Na₂SO₄, filtered, and the filtrate was concentrated under reduced pressure. The residue was purified by trituration with Et₂O (50 mL). The precipitated solids were collected by filtration and washed with Et₂O (3×20 mL) to afford the title compound (5 g, 79% yield) as a yellow solid. LC/MS (ESI, m/z): [(M+H)]⁺=196.1.

Step 2—4-[(E)-2-(dimethylamino) ethenyl]-5-nitropyridin-2-amine. To a stirred solution of N-(4-methyl-5-nitropyridin-2-yl)acetamide (5 g, 30 mmol) in DMF (50 mL) was added DMF-DMA (7.5 mL, 56 mmol) dropwise at rt under nitrogen atmosphere. The resulting mixture was stirred for an additional 3 h at 90° C. On completion, the mixture was cooled to rt and concentrated under vacuum. The residue was purified by trituration with Et₂O (50 mL). The precipitated solids were collected by filtration and washed with PE (3×50 mL) to afford the title compound (4.7 g, 88% yield) as an orange solid. LC/MS (ESI, m/z): [(M+H)]⁺=209.1.

Step 3—N-{1H-pyrrolo[2,3-c]pyridin-5-yl}acetamide. To a stirred solution of 4-[(E)-2-(dimethylamino)ethenyl]-5-nitropyridin-2-amine (4 g, 20 mmol) in MeOH (570 mL) and DCM (114 mL) were added Pd/C (5.71 g, 53.7 mmol) and AcOH (40.00 mL) at rt. The resulting mixture was stirred for overnight at rt under hydrogen atmosphere. On completion, the mixture was filtered, and the filter cake was washed with MeOH (5×100 mL). The filtrate was concentrated under reduced pressure. The residue was purified by silica gel column chromatography, eluted with CH₂Cl₂/MeOH (30:1), to afford the title compound (3 g, 89% yield) as a yellow solid. LC/MS (ESI, m/z): [(M+H)]⁺=176.1.

Step 4—N-{3-bromo-1H-pyrrolo[2,3-c]pyridin-5-yl}acetamide. To a stirred solution of N-{1H-pyrrolo[2,3-c]pyridin-5-yl}acetamide (3 g, 20 mmol) in DMF (26 mL) was added NBS (3.35 g, 18.8 mmol) at rt and the mixture was stirred for 2 h at rt. On completion, the reaction was quenched by the addition of sat, sodium sulfite (aq.) (2 mL) at rt. The resulting mixture was filtered, the filter cake was washed with MeOH (3×100 mL). The filtrate was concentrated under reduced pressure. The residue was purified by silica gel column chromatography, eluted with CH₂Cl₂/MeOH (15:1), to afford the title compound (4 g, 92% yield) as a grey solid. LC/MS (ESI, m/z): [(M+H)]⁺=254.0, 256.0.

Step 5—Tert-butyl 3-bromo-5-acetamidopyrrolo[2,3-c]pyridine-1-carboxylate. To a stirred mixture of N-{3-bromo-1H-pyrrolo[2,3-c]585yridine-5-yl}acetamide (8.00 g, 31.5 mmol) and (Boc)₂O (8.25 g, 37.8 mmol) in ACN (160 mL) was added DMAP (0.38 g, 3.15 mmol) at rt under nitrogen atmosphere and the mixture was stirred for 1 h at rt under nitrogen atmosphere. On completion, the mixture was concentrated under reduced pressure. The residue was purified by silica gel column chromatography, eluted with 1%-70% ethyl acetate in petroleum ether, to afford the title compound (9.50 g, 85% yield) as a yellow solid. LC/MS (ESI, m/z): [(M+H)]⁺=354.0, 356.0.

Step 6—Tert-butyl 5-acetamido-3-(4,4,5,5-tetramethyl-1,3,2-dioxaborolan-2-yl)pyrrolo[2,3-c]pyridine-1-carboxylate. To a stirred mixture of tert-butyl 3-bromo-5-acetamidopyrrolo[2,3-c]pyridine-1-carboxylate (5.00 g, 14.1 mmol) and B₂Pin₂ (7.17 g, 28.2 mmol) in dioxane (50 mL) were added KOAc (4.16 g, 42.4 mmol) and Pd(dppf)Cl₂CH₂Cl₂ (1.15 g, 1.41 mmol) at rt under nitrogen atmosphere. The resulting mixture was stirred for 2 h at 80° C. under nitrogen atmosphere. On completion, the mixture was cooled to rt and concentrated under reduced pressure. The residue was purified by silica gel column chromatography, eluted with 1%-30% ethyl acetate in petroleum ether, to afford the title compound (5.5 g, 97% yield) as a yellow solid. LC/MS (ESI, m/z): [(M+H)]⁺=402.2.

### N-{3-[5-(1,3-dioxolan-2-yl)-6′-[(3S)-3-methoxyoxolan-3-yl]-[2,4′-bipyridin]-2′-yl]-1-methylpyrrolo[2,3-c]pyridin-5-yl}acetamide (Intermediate NM) and N-{3-[5-(1,3-dioxolan-2-yl)-6′-[(3R)-3-methoxyoxolan-3-yl]-[2,4′-bipyridin]-2′-yl]-1-methylpyrrolo[2,3-c]pyridin-5-yl}acetamide (Intermediate NN)

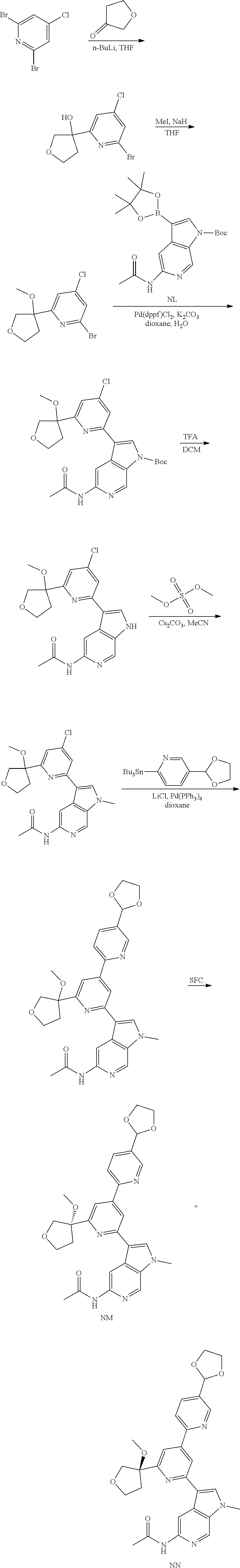

Step 1—3-(6-Bromo-4-chloropyridin-2-yl)oxolan-3-ol. To a stirred solution of 2,6-dibromo-4-chloropyridine (40.00 g, 147.4 mmol) in THF (400 mL) was added n-BuLi in hexane (64.86 mL, 162.2 mmol) at −78° C. under nitrogen atmosphere. The resulting mixture was stirred for 0.5 h at −78° C. under nitrogen atmosphere. To the above mixture was added dihydrofuran-3-one (15.23 g, 176.9 mmol) at −78° C. The resulting mixture was stirred for an additional 3 h at rt. On completion, the reaction was quenched by the addition of MeOH (400 mL) at 0° C. The resulting mixture was concentrated under reduced pressure. The residue was purified by silica gel column chromatography, eluted with PE/EA 2:1), to afford the title compound (21.7 g, 53% yield) as a brown yellow solid. LC/MS (ESI, m/z): [(M+H)]⁺=278.0, 280.0.

Step 2—2-Bromo-4-chloro-6-(3-methoxyoxolan-3-yl)pyridine. To a stirred solution of 3-(6-bromo-4-chloropyridin-2-yl)oxolan-3-ol (11.5 g, 41.3 mmol) in THF (150 mL) was added NaH (3.30 g, 82.6 mmol) at 0° C. The resulting mixture was stirred for 30 min at rt. To the above mixture was added Mel (8.79 g, 61.932 mmol) at 0° C. The resulting mixture was stirred for an additional 16 h at rt. On completion, the reaction was quenched by the addition of NH4Cl (aq.) (300 mL) at 0° C. The resulting mixture was extracted with EtOAc (3×300 mL). The combined organic layers were dried over anhydrous Na₂SO₄, filtered, and the filtrate was concentrated under reduced pressure. The residue was purified by silica gel column chromatography, eluted with PE/EA (5:1), to afford the title compound (11 g, 91% yield) as a colorless oil. LC/MS (ESI, m/z): [(M+H)]⁺=292.0, 294.0.

Step 3—Tert-butyl 3-[4-chloro-6-(3-methoxyoxolan-3-yl)pyridin-2-yl]-5-acetamidopyrrolo[2,3-c]pyridine-1-carboxylate. To a stirred mixture of 2-bromo-4-chloro-6-(3-methoxyoxolan-3-yl)pyridine (5 g, 20 mmol) and tert-butyl 5-acetamido-3-(4,4,5,5-tetramethyl-1,3,2-dioxaborolan-2-yl)pyrrolo[2,3-c]pyridine-1-carboxylate (6.86 g, 17.1 mmol, Intermediate NL) in dioxane (50 mL) and H₂O (10 mL) were added K₂CO₃ (7.09 g, 51.3 mmol) and Pd(dppf)C₁₋₂CH₂Cl₂ (1.39 g, 1.71 mmol) at rt under nitrogen atmosphere. The resulting mixture was stirred for an additional 2 h at 90° C. On completion, the mixture was cooled to it and concentrated under reduced pressure. The residue was purified by silica gel column chromatography, eluted with CH₂Cl₂/EA (1:4), to afford the title compound (5.1 g, 61% yield) as a yellow solid. LC/MS (ESI, m/z): [(M+H)]⁺=487.2.

Step 4—N-{3-[4-chloro-6-(3-methoxyoxolan-3-yl)pyridin-2-yl]-1H-pyrrolo[2,3-c]pyridin-5-yl}acetamide trifluoroacetate. To a stirred mixture of tert-butyl 3-[4-chloro-6-(3-methoxyoxolan-3-yl)pyridin-2-yl]-5-acetamidopyrrolo[2,3-c]pyridine-1-carboxylate (5.1 g, 11 mmol) in DCM (50 mL) was added TFA (25 mL) dropwise at rt under nitrogen atmosphere and the mixture was stirred for 1 h at rt. On completion, the mixture was concentrated under reduced pressure. The residue was purified by trituration with Et₂O (50 mL) to give the title compound (4.6 g, 88% yield) as a white solid. LC/MS (ESI, m/z): [(M+H)]⁺=387.2.

Step 5—N-{3-[4-chloro-6-(3-methoxyoxolan-3-yl)pyridin-2-yl]-1-methylpyrrolo[2,3-c]pyridin-5-yl}acetamide. To a stirred mixture of N-{3-[4-chloro-6-(3-methoxyoxolan-3-yl)pyridin-2-yl]-1H-pyrrolo[2,3-c]pyridin-5-yl}acetamide trifluoroacetate (4.6 g, 9.2 mmol) and dimethyl sulfate (2.32 g, 18.4 mmol) in ACN (100 mL) was added Cs₂CO₃ (2.10 g, 27.6 mmol) at rt under nitrogen atmosphere and the mixture was stirred for 1 h at rt. On completion, the mixture was concentrated under reduced pressure. The residue was purified by silica gel column chromatography, eluted with CH₂Cl₂/(EtOH/EA) (1:(1:3)), to afford the title compound (3.6 g, 98% yield) as a white solid. LC/MS (ESI, m/z): [(M+H)]f=401.1.

Step 6—N-{3-[5-(1,3-dioxolan-2-yl)-6′-(3-methoxyoxolan-3-yl)-[2,4′-bipyridin]-2′-yl]-1-methylpyrrolo[2,3-c]pyridin-5-yl}acetamide. To a stirred mixture of N-{3-[4-bromo-6-(3-methoxyoxolan-3-yl)pyridin-2-yl]-1-methylpyrrolo[2,3-c]pyridin-5-yl}acetamide (500 mg, 1.12 mmol,) LiCl (47.60 mg, 1.123 mmol) and 5-(1,3-dioxolan-2-yl)-2-(tributylstannyl)pyridine (988.54 mg, 2.246 mmol) in dioxane (9 mL) was added Pd(PPh₃)₄ (129.75 mg, 0.112 mmol) at rt under nitrogen atmosphere. The resulting mixture was stirred for 16 h at 100° C. under nitrogen atmosphere. On completion, the mixture was cooled to rt and concentrated under reduced pressure. The residue was purified by silica gel column chromatography, eluted with PE/EA/EtOH (4:3:1), and concentrated under reduced pressure. The crude product (510 mg) was further purified by Prep-HPLC (Column: X^{B} ridge Prep Phenyl OBD Column 19*250 mm, 5m; Mobile Phase A: Water(10 mmol/L NH₄HCO₃), Mobile Phase B: ACN; Flow rate: 20 mL/min; Gradient: 24% B to 34% B in 10 min; Wave Length: 254/220 nm; RT1(min): 14.33-16.12) and concentrated under reduced pressure to afford the title compound (333 mg, 58% yield) as an off-white solid. LC/MS (ESI, m/z): [(M+H)]⁺=516.1.

Step 7—N-{3-[5-(1,3-dioxolan-2-yl)-6′-[(3R)-3-methoxyoxolan-3-yl]-[2,4′-bipyridin]-2′-yl]-1-methylpyrrolo[2,3-c]pyridin-5-yl}acetamide and N-{3-[5-(1,3-dioxolan-2-yl)-6′-[(3S)-3-methoxyoxolan-3-yl]-[2,4′-bipyridin]-2′-yl]-1-methylpyrrolo[2,3-c]pyridin-5-yl}acetamide. The racemic mixture of N-{3-[5-(1,3-dioxolan-2-yl)-6′-(3-methoxyoxolan-3-yl)-[2,4′-bipyridin]-2′-yl]-1-methylpyrrolo[2,3-c]pyridin-5-yl}acetamide (333 mg) was separated by chiral resolution (Column: CHIRALPAK OD 2*25 cm, 5 μm; Mobile Phase A: Hex(0.5% 2M NH₃-MeOH)—HPLC, Mobile Phase B: IPA-HPLC; Flow rate: 20 mL/min; Gradient: isocratic 50; Wave Length: 220/254 nm; RT1(min): 27.93; RT2(min): 38.91. The fractions of slower eluting peak was collected and concentrated under vacuum to afford to afford N-{3-[5-(1,3-dioxolan-2-yl)-6′-[(3R)-3-methoxyoxolan-3-yl]-[2,4′-bipyridin]-2′-yl]-1-methylpyrrolo[2,3-c]pyridin-5-yl}acetamide (87 mg, 26% yield) as a white solid. The faster fractions of eluting peak was collected and concentrated under vacuum to afford N-{3-[5-(1,3-dioxolan-2-yl)-6′-[(3S)-3-methoxyoxolan-3-yl]-[2,4′-bipyridin]-2′-yl]-1-methylpyrrolo[2,3-c]pyridin-5-yl}acetamide (90 mg, 27% yield) as a white solid. LC/MS (ESI, m/z): [(M+H)]⁺=516.2 for both isomers. The absolute stereochemistry of the enantiomers was assigned arbitrarily.

### N-(3-{5-formyl-6′-[(3R)-3-methoxyoxolan-3-yl]-[2,4′-bipyridin]-2′-yl}-1-methylpyrrolo[2,3-c]pyridin-5-yl)acetamide (Intermediate NO)

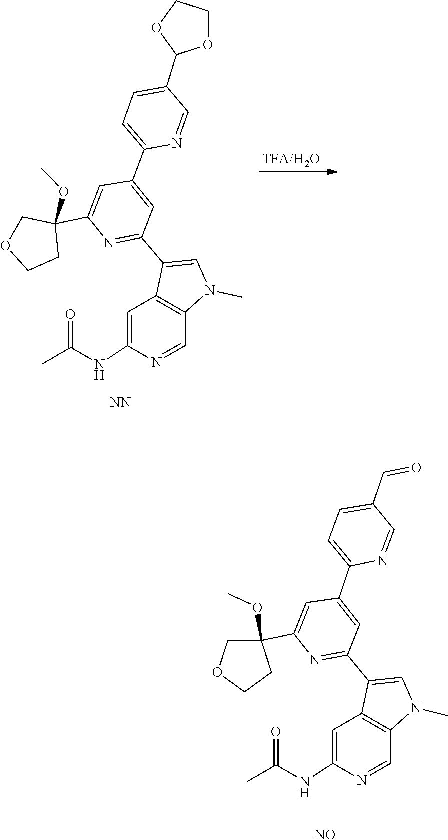

To a stirred mixture of N-{3-[5-(1,3-dioxolan-2-yl)-6′-[(3R)-3-methoxyoxolan-3-yl]-[2,4′-bipyridin]-2′-yl]-1-methylpyrrolo[2,3-c]pyridin-5-yl}acetamide (30 mg, 0.06 mmol, Intermediate NN) in H2O (1 mL) was added TFA (1 mL) at rt under nitrogen atmosphere. The resulting mixture was stirred for 1 h at 50° C. under nitrogen atmosphere. On completion, the mixture was cooled to rt and concentrated under reduced pressure to give the title compound (30 mg) as a yellow oil. LC/MS (ESI, m/z): [(M+H)]⁺=472.3 6-[4-(5-Formylpyridin-2-yl)-2,3-dihydroindol-1-yl]-N-[(1R,2R)-2-methoxycyclobutyl]-8-(methylamino)imidazo[1,2-b]pyridazine-3-carboxamide (Intermediate NP)

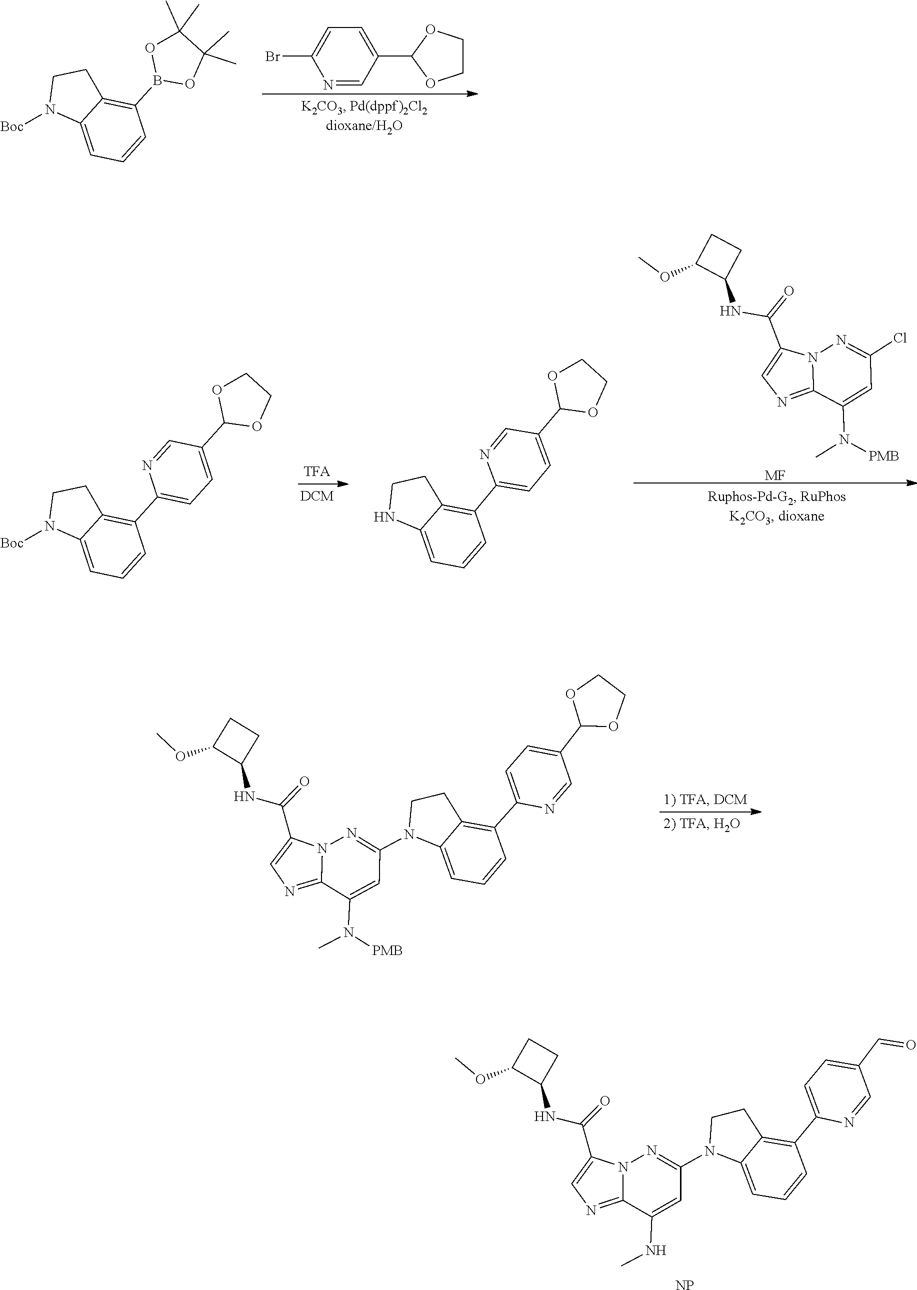

Step 1—Tert-butyl 4-[5-(1,3-dioxolan-2-yl)pyridin-2-yl]-2,3-dihydroindole-1-carboxylate. To a stirred mixture of tert-butyl 4-(4,4,5,5-tetramethyl-1,3,2-dioxaborolan-2-yl)indoline-1-carboxylate (40.00 g, 115.9 mmol, Intermediate MA) and 2-bromo-5-(1,3-dioxolan-2-yl)pyridine (26.65 g, 115.9 mmol, CAS #220904-17-6) in 1,4-dioxane (400 mL) and H₂O (80 mL) were added K₂CO₃ (48.04 g, 347.6 mmol), Pd(dppf)C₁₋₂·CH₂Cl₂ (9.44 g, 11.6 mmol) at rt under nitrogen atmosphere. The resulting mixture was stirred for 2 h at 90° C. under nitrogen atmosphere. On completion, the mixture was cooled to it and concentrated under reduced pressure. The residue was purified by silica gel column chromatography, eluted with PE/EA (3:1) to afford the title compound (21.00 g, 49% yield) as a yellow solid. LC/MS (ESI, m/z): [(M+H)]⁺=369.2.

Step 2—4-[5-(1,3-Dioxolan-2-yl)pyridin-2-yl]-2,3-dihydro-TH-indole. A mixture of tert-butyl 4-[5-(1,3-dioxolan-2-yl)pyridin-2-yl]-2,3-dihydroindole-1-carboxylate (21.00 g, 57.00 mmol) in DCM (210 mL) and TFA (70 mL) was stirred for 1 h at rt. On completion, the mixture was neutralized to pH 8 with NaOH and diluted with water (210 mL). The aqueous layer was extracted with EtOAc (3×100 mL). The combined organic layers and dried over anhydrous Na₂SO₄, filtered, and the filtrate was concentrated under reduced pressure to afford the title compound (18.00 g) as a yellow solid. LC/MS (EST m/z): [(M+H)]⁺=269.2.

Step 3—6-{4-[5-(1,3-Dioxolan-2-yl)pyridin-2-yl]-2,3-dihydroindol-1-yl}-N-[(1R,2R)-2-methoxycyclobutyl]-8-{[(4-methoxyphenyl)methyl](methyl)amino}imidazo[1,2-b]pyridazine-3-carboxamide. To a stirred mixture of 6-chloro-N-[(1R,2R)-2-methoxycyclobutyl]-8-{[(4-methoxyphenyl)methyl](methyl)amino}imidazo[1,2-b]pyridazine-3-carboxamide (300 mg, 0.698 mmol, Intermediate MF), 4-[5-(1,3-dioxolan-2-yl)pyridin-2-yl]-2,3-dihydro-1H-indole (224.68 mg, 0.838 mmol) and K₂CO₃ (289.33 mg, 2.094 mmol) in dioxane (6 mL) were added RuPhos (32.56 mg, 0.070 mmol) and 2nd Generation RuPhos precatalyst (54.20 mg, 0.070 mmol) in portions at rt under nitrogen atmosphere. The resulting mixture was stirred for 2 h at 110° C. under nitrogen atmosphere. On completion, the mixture was cooled to rt and concentrated under reduced pressure. The residue was purified by reversed-phase flash chromatography (Column: Spherical C18, 20˜40 μm, 330 g; Mobile Phase A: Water(plus 10 mM NH4HCO₃), Mobile Phase B: ACN; Flow rate: 80 mL/min; Gradient (B %): 5%60%, 4 min; 60%˜80%, 20 min; 80%˜95%; 2 min; 95%, 5 min; Detector: 254 nm; the fractions containing desired product were collected at 80% B) and concentrated under reduced pressure to afford the title compound (450 mg, 97% yield) as a yellow solid. LC/MS (ESI, m/z): [(M+H)]⁺=662.3.

Step 4—6-[4-(5-Formylpyridin-2-yl)-2,3-dihydroindol-1-yl]-N-[(1R,2R)-2-methoxycyclobutyl]-8-(methylamino)imidazo[1,2-b]pyridazine-3-carboxamide. To a solution of 6-{4-[5-(1,3-dioxolan-2-yl)pyridin-2-yl]-2,3-dihydroindol-1-yl}-N-[(1R,2R)-2-methoxycyclobutyl]-8-{[(4-methoxyphenyl)methyl](methyl)amino}imidazo[1,2-b]pyridazine-3-carboxamide (450 mg, 0.680 mmol) in DCM (5 mL) was added TFA (1 mL) dropwise at rt and the mixture was stirred for 1 h at rt. Then the mixture was concentrated under reduced pressure. To the above mixture were added H₂O (5 mL) and TFA (5 mL) dropwise at rt. The resulting mixture was stirred for an additional 2 h at 50° C. On completion, the mixture was cooled to rt and neutralized to pH 8 with saturated Na₂CO₃ (aq.). The precipitated solids were collected by filtration and washed with water (3×10 mL) to afford the title compound (450 mg) as a yellow solid. LC/MS (ESI, m/z): [(M+H)]f=498.2.

### 1-{2-Chloro-5-[methyl(piperidin-4-yl)amino]phenyl}-1,3-diazinane-2,4-dione trifluoroacetate (Intermediate NQ)

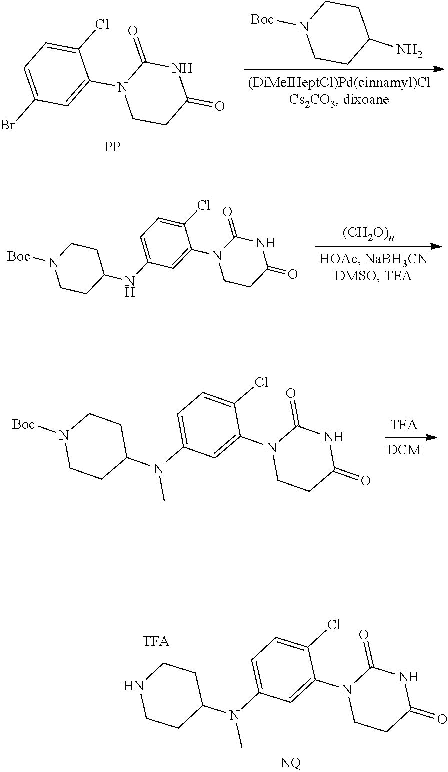 [SMILES: O=C1CCN(c2cc(Br)ccc2Cl)C(=O)N1 ; CC(C)(C)OC(=O)N1CCC(N)CC1 ; "PP" ; CC(C)(C)OC(=O)N1CCC(Nc2ccc(Cl)c(N3CCC(=O)NC3=O)c2)CC1 ; CN(c1ccc(Cl)c(N2CCC(=O)NC2=O)c1)C1CCN(C(=O)OC(C)(C)C)CC1 ; CN(c1ccc(Cl)c(N2CCC(=O)NC2=O)c1)C1CCNCC1]

Step 1—Tert-butyl 4-{[4-chloro-3-(2,4-dioxo-1,3-diazinan-1-yl)phenyl]amino}piperidine-1-carboxylate. To a stirred mixture of 1-(5-bromo-2-chlorophenyl)dihydropyrimidine-2,4(1H,3H)-dione (700 mg, 2.31 mmol, Intermediate PP) and tert-butyl 4-aminopiperidine-1-carboxylate (508.06 mg, 2.537 mmol) in dioxane (10 mL) was added Cs₂CO₃ (2.25 g, 6.92 mmol) and (prop-2-en-1-yl)benzene; {1-[2,6-bis(2,6-dimethylheptan-4-yl)phenyl]-4,5-dichloro-3-{2-[2,8-dimethyl-3,7-bis(propan-2-yl)nonan-5-yl]-6-(2,6-dimethylheptan-4-yl)phenyl}-2,3-dihydro-1H-imidazol-2-yl}(chloro)palladium (269.21 mg, 0.231 mmol) at rt under nitrogen atmosphere. The resulting mixture was stirred for 1 h at 100° C. under nitrogen atmosphere. On completion, the mixture was cooled to rt and concentrated under reduced pressure. The residue was purified by reverse phase flash chromatography (Column: WelFlash™ C18-I, 20-40 g, 330 g; Eluent A: Water (plus 10 mmol/LNH₄HCO₃); Eluent B: ACN; Gradient: 35%-55% B in 20 min; Flow rate: 80 mL/min; Detector: 220/254 nm; desired fractions were collected at 45% B) and concentrated under reduced pressure to afford the title compound (500 mg, 51% yield) as an off-white solid. LC/MS (ESI, m/z): [(M-56+H)]⁺=367.1.

Step 2—Tert-butyl 4-{[4-chloro-3-(2,4-dioxo-1,3-diazinan-1-yl)phenyl](methyl)amino}piperidine-1-carboxylate. To a stirred mixture of tert-butyl 4-{[4-chloro-3-(2,4-dioxo-1,3-diazinan-1-yl)phenyl]amino}piperidine-1-carboxylate (500 mg, 1.18 mmol) and TEA (0.49 mL, 3.55 mmol) in DMSO (10 mL) were added paraformaldehyde (159.75 mg, 1.773 mmol) and HOAc (0.34 mL, 5.91 mmol) at rt. The resulting mixture was stirred for 30 min at 50° C. under nitrogen atmosphere. The mixture was allowed to cool down to rt. To the above mixture was added NaBH₃CN (148.59 mg, 2.364 mmol) at rt. Then the resulting mixture was stirred for 2 h at 50° C. On completion, the mixture was cooled to rt and concentrated under reduced pressure. The residue was purified by reverse phase Flash chromatography (Column: WelFlash™ C18-I, 20-40 μm, 330 g; Eluent A: Water (plus 10 mmol/L NH4HCO₃); Eluent B: ACN; Gradient: 35%-50% B in 25 min; Flow rate: 60 mL/min; Detector: 220/254 nm; desired fractions were collected at 42% B) and concentrated under reduced pressure to afford the title compound (470 mg, 91% yield) as a white solid. LC/MS (ESL m/z): [(M+H)]⁺=437.1.

Step 3—1-{2-Chloro-5-[methyl(piperidin-4-yl)amino]phenyl}-1,3-diazinane-2,4-dione trifluoroacetate. To a stirred solution of tert-butyl 4-{[4-chloro-3-(2,4-dioxo-1,3-diazinan-1-yl)phenyl](methyl)amino}piperidine-1-carboxylate (470 mg, 1.08 mmol) in DCM (10 mL) was added TFA (3 mL) dropwise at rt. The resulting mixture was stirred for 1 h at rt. On completion, the mixture was concentrated under reduced pressure. The residue was purified by trituration with Et₂O (20 mL) to afford the title compound (500 mg) as a white solid. LC/MS (ESI, m/z): [(M+H)]⁺=337.2.

### 1-(5-{[1,4′-bipiperidin]-4-yl(methyl)amino}-2-chlorophenyl)-1,3-diazinane-2,4-dione trifluoroacetate (Intermediate NR)

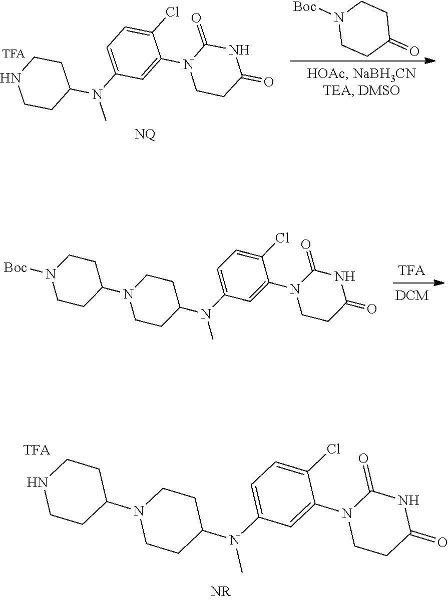 [SMILES: CN(c1ccc(Cl)c(N2CCC(=O)NC2=O)c1)C1CCNCC1 ; CC(C)(C)OC(=O)N1CCC(=O)CC1 ; CN(c1ccc(Cl)c(N2CCC(=O)NC2=O)c1)C1CCN(C2CCN(C(=O)OC(C)(C)C)CC2)CC1 ; CN(c1ccc(Cl)c(N2CCC(=O)NC2=O)c1)C1CCN(C2CCNCC2)CC1]

Step 1—Tert-butyl 4-{[4-chloro-3-(2,4-dioxo-1,3-diazinan-1-yl)phenyl](methyl)amino}-[1,4′-bipiperidine]-1′-carboxylate. To a stirred mixture of 1-{2-chloro-5-[methyl(piperidin-4-yl)amino]phenyl}-1,3-diazinane-2,4-dione trifluoroacetate (150 mg, 0.345 mmol, Intermediate NQ) and TEA (0.14 mL, 1.04 mmol) in DMSO (2 mL) were added tert-butyl 4-oxopiperidine-1-carboxylate (82 mg, 0.41 mmol) and HOAc (0.10 mL, 1.7 mmol) at rt under nitrogen atmosphere. The resulting mixture was stirred for 30 min at 50° C. under nitrogen atmosphere. The mixture was allowed to cool down to rt. To the above mixture was added NaBH₃CN (65 mg, 1.034 mmol) and the mixture was stirred for additional 2 h at 50° C. On completion, the mixture was cooled to rt and concentrated under reduced pressure. The residue was purified by reverse phase flash chromatography (Column: WelFlash™ C18-I, 20-40 μm, 120 g; Eluent A: Water (plus 10 mmol/L NH4HCO₃); Eluent B: ACN; Gradient: 35%-55% B in 15 min; Flow rate: 50 mL/min; Detector: 220/254 nm; desired fractions were collected at 50% B) and concentrated under reduced pressure to afford the title compound (140 mg, 78% yield) as a white solid. LC/MS (ESI, m/z): [(M+H)]⁺=520.4.

Step 2—1-(5-{[1,4′-Bipiperidin]-4-yl(methyl)amino}-2-chlorophenyl)-1,3-diazinane-2,4-dione trifluoroacetate. To a stirred mixture of tert-butyl 4-{[4-chloro-3-(2,4-dioxo-1,3-diazinan-1-yl)phenyl](methyl)amino}-[1,4′-bipiperidine]-1′-carboxylate (140 mg, 0.27 mmol) in DCM (2 mL) was added TFA (1 mL) at rt under nitrogen atmosphere and the mixture was stirred for 1 h at rt under nitrogen atmosphere. On completion, the mixture was concentrated under reduced pressure. The residue was purified by trituration with Et₂O (10 mL) to afford the title compound (139 mg) as a white solid. LC/MS (ESI, m/z): [(M+H)]⁺=420.3.

### Tert-butyl 2-(3,3-difluoropiperidin-4-yl)-2,7-diazaspiro[3.5]nonane-7-carboxylate (Intermediate NS)

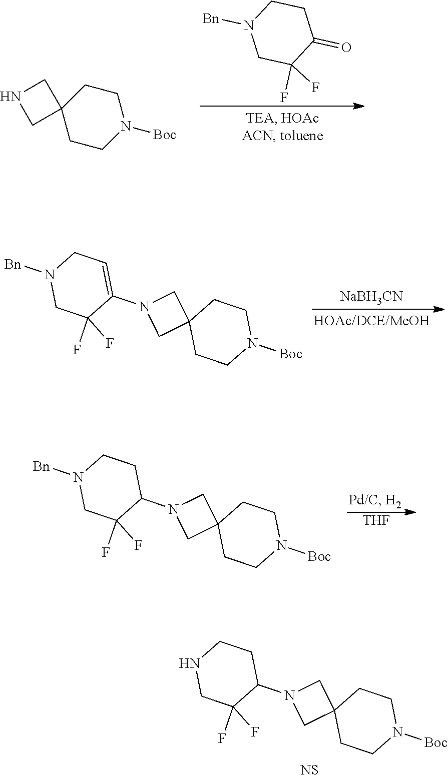

Step 1—Tert-butyl 2-(1-benzyl-3,3-difluoro-1,2,3,6-tetrahydropyridin-4-yl)-2,7-diazaspiro[3.5]nonane-7-carboxylate. To a stirred solution of tert-butyl 2,7-diazaspiro[3.5]nonane-7-carboxylate (20 g, 88.370 mmol) and TEA (17.88 g, 176.7 mmol, CAS #1023301-84-9) in ACN (20 mL) and toluene (120 mL) was added 1-benzyl-3,3-difluoropiperidin-4-one (29.86 g, 132.6 mmol, CAS #1039741-54-2) and HOAc (15.92 g, 265.1 mmol) in turns at rt under nitrogen atmosphere. The resulting mixture was stirred for 16 h at 110° C. under nitrogen atmosphere. On completion, the mixture was cooled to rt and concentrated under reduced pressure. The resulting mixture was extracted with EtOAc (3×300 mL). The combined organic layers were washed with water (800 mL). The combined organic layers were washed with brine (800 mL), dried over anhydrous Na₂SO₄. After filtration, the filtrate was concentrated under reduced pressure to give the title compound (30 g, 78% yield) as a yellow oil. LC/MS (ESI, m/z): [(M+H)]⁺=434.3.

Step 2—Tert-butyl 2-(1-benzyl-3,3-difluoropiperidin-4-yl)-2,7-diazaspiro[3.5]nonane-7-carboxylate. To a stirred solution of tert-butyl 2-(1-benzyl-3,3-difluoro-1,2,3,6-tetrahydropyridin-4-yl)-2,7-diazaspiro[3.5]nonane-7-carboxylate (40 g, 90 mmol) and HOAc (10 mL) in DCE (100 mL) and MeOH (100 mL) was added NaBH₃CN (28.99 g, 461.3 mmol) dropwise at rt. The resulting mixture was stirred for 16 h at rt under nitrogen atmosphere. On completion, the mixture was concentrated under reduced pressure. The mixture was purified by silica gel column chromatography (Mobile Phase A: PE, Mobile Phase B: EA; Gradient: 50% to 100% B in 25 min. 254 nm; the fractions containing the desired product were collected at 74% B) and concentrated under reduced pressure to afford the title compound (3.8 g, 10% yield) as a white solid. LC/MS (ESI, m/z): [(M+H)]⁺=436.1.

Step 3—Tert-butyl 2-(3,3-difluoropiperidin-4-yl)-2,7-diazaspiro[3.5]nonane-7-carboxylate. To a solution of tert-butyl 2-(1-benzyl-3,3-difluoropiperidin-4-yl)-2,7-diazaspiro[3.5]nonane-7-carboxylate (800 mg, 1.84 mmol) in THF (10 mL) was added 10 wt % Pd/C (1 g, 10 mmol) under nitrogen atmosphere. The reaction system was degassed under vacuum and purged with H2 several times, then it was hydrogenated under H2 balloon (˜1 atm) at 25° C., for 2 h. After completion of the reaction, Pd/C was filtered off through celite and the filter cake was washed with THF (3×30 mL). The corresponding filtrate was concentrated under reduced pressure to afford the title compound (400 mg, 63% yield) as a white solid. LC/MS (ESI, m/z): [(M+H)]f=346.3.

### 6-(4-((3,3-difluoro-4-(2,7-diazaspiro [3.5]nonan-2-yl)piperidin-1-yl)methyl)indolin-1-yl)-N-((1R,2R)-2-methoxycyclobutyl)-8-(methylamino)imidazo[1,2-b]pyridazine-3-carboxamide trifluoroacetate (Intermediate NT)

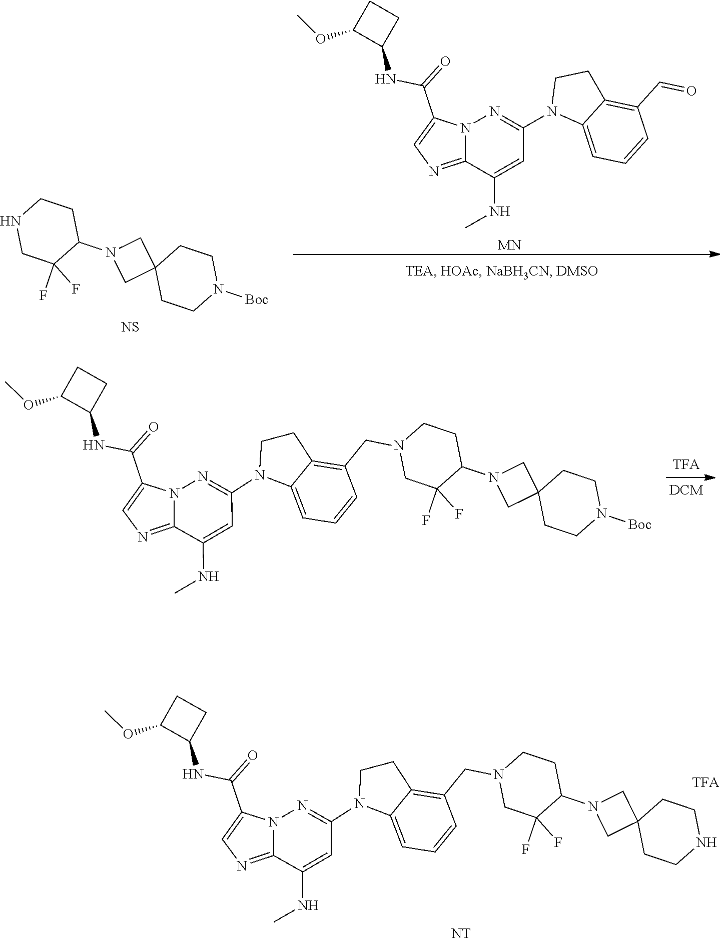

Step 1—Tert-butyl 2-(3,3-difluoro-1-((1-(3-(((1R,2R)-2-methoxycyclobutyl)carbamoyl)-8-(methylamino)imidazo[1,2-b]pyridazin-6-yl)indolin-4-yl)methyl)piperidin-4-yl)-2,7-diazaspiro[3.5]nonane-7-carboxylate. To a stirred solution of tert-butyl 2-(3,3-difluoropiperidin-4-yl)-2,7-diazaspiro[3.5]nonane-7-carboxylate (150 mg, 0.43 mmol, Intermediate NS) and TEA (87.88 mg, 0.86 mmol) in DMSO (5 mL) was added 6-(4-formyl-2,3-dihydroindol-1-yl)-N-[(1R,2R)-2-methoxycyclobutyl]-8-(methylamino)imidazo[1,2-b]pyridazine-3-carboxamide (182.58 mg, 0.43 mmol, Intermediate MN) and HOAc (78.23 mg, 1.30 mmol) at rt. To the above mixture was added NaBH₃CN (81.86 mg, 1.30 mmol) over 2 min at rt. The resulting mixture was stirred for an additional 1 h at 50° C. After completion of the reaction, the reaction mixture was cooled to rt and purified directly by reverse phase flash chromatography (Column: WelFlash™ C18-I, 20-40 m, 120 g; Eluent A: Water (10 mmol/L NH4HCO₃); Eluent B: ACN; Gradient: 60%-80% B in 25 min; Flow rate: 80 mL/min; Detector: 220/254 nm; desired fractions were collected at 70% B) and concentrated under reduced pressure to afford the title compound (120 mg, 36% yield) as a yellow solid. LC/MS (ESI, m/z): [(M+H)]⁺=750.3.

Step 2—6-(4-((3,3-Difluoro-4-(2,7-diazaspiro[3.5]nonan-2-yl)piperidin-1-yl)methyl)indolin-1-yl)-N-((1R,2R)-2-methoxycyclobutyl)-8-(methylamino)imidazo[1,2-b]pyridazine-3-carboxamide trifluoroacetate. To a stirred solution of tert-butyl 2-(3,3-difluoro-1-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}piperidin-4-yl)-2,7-diazaspiro[3.5]nonane-7-carboxylate (120 mg, 0.16 mmol) in DCM (6 mL) was added TFA (2 mL) dropwise at rt. The resulting mixture was stirred for 1 h at rt. On completion, the mixture was concentrated under reduced pressure. The mixture was purified by trituration with Et₂O (10 mL) to afford the title compound (119 mg, 99% yield) as a white solid. LC/MS (ESI, m/z): [(M+H)]+=650.4.

### (R)-6-chloro-N-(2,2-difluorocyclobutyl)-8-((4-methoxybenzyl)(methyl)amino)imidazo[1,2-b]pyridazine-3-carboxamide (Intermediate NU) and (S)-6-chloro-N-(2,2-difluorocyclobutyl)-8-((4-methoxybenzyl)(methyl)amino)imidazo[1,2-b]pyridazine-3-carboxamide (Intermediate NV)

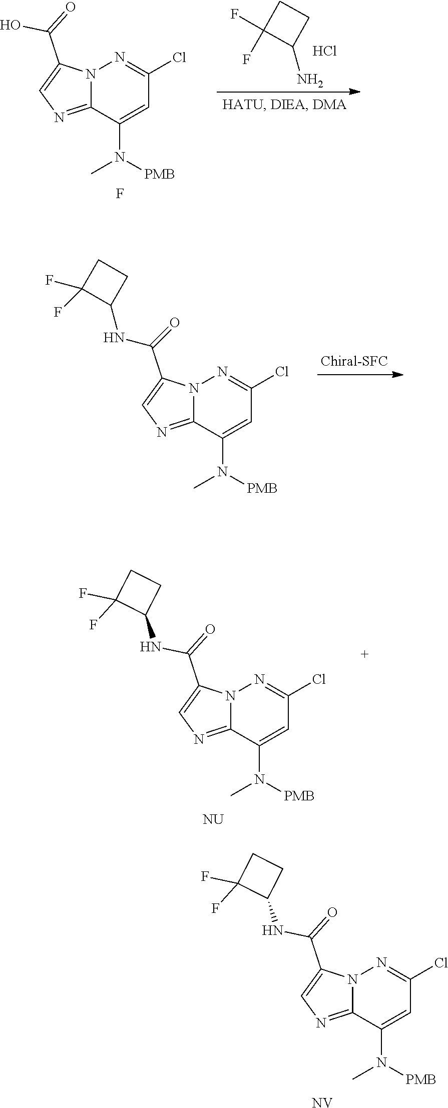

Step 1—6-chloro-N-(2,2-difluorocyclobutyl)-8-((4-methoxybenzyl)(methyl)amino)imidazo[1,2-, b]pyridazine-3-carboxamide. A solution of 6-chloro-8-((4-methoxybenzyl)(methyl)amino)imidazo[1,2-b]pyridazine-3-carboxylic acid (5.31 g, 15.3 mmol, Intermediate F) and HATU (6.36 g, 16.7 mmol) in DMA (30 mL) was treated with DIEA (5.40 g, 41.8 mmol) for 10 min at rt followed by the addition of 2,2-difluorocyclobutan-1-amine hydrochloride (2.00 g, 13.9 mmol) in portions at rt. The resulting mixture was stirred for 1 h at rt. On completion, the mixture was purified by reversed-phase flash chromatography (Column: Spherical C18, 20-40 μm, 330 g; Mobile Phase A: Water(plus 10 mM NH₄HCO₃), Mobile Phase B: ACN; Flow rate: 80 mL/min; Gradient (B %): 5%˜55%, 4 min; 55%˜75%, 20 min; 75%˜95%; 2 min; 95%, 5 min; Detector: 254 nm; the fractions containing desired product were collected at 70% B) and concentrated under reduced pressure to afford the title compound (5.90 g, 97% yield) as a yellow solid. LC/MS (ESI, m/z): [(M+1)]⁺=436.2.

Step 2—(R)-6-chloro-N-(2,2-difluorocyclobutyl)-8-((4-methoxybenzyl)(methyl)amino)imidazo[1,2-b]pyridazine-3-carboxamide and (S)-6-chloro-N-(2,2-difluorocyclobutyl)-8-((4-methoxybenzyl)(methyl)amino)imidazo[1,2-b]pyridazine-3-carboxamide. The racemic 6-chloro-N-(2,2-difluorocyclobutyl)-8-((4-methoxybenzyl)(methyl)amino)imidazo[1,2-b]pyridazine-3-carboxamide (5.90 g) was separated by Prep-chiral-HPLC (Column: CHIRALPAK AD-H, 5*25 cm, 5 μm; Mobile Phase A: CO₂, Mobile Phase B: MeOH(0.1% 2M NH₃-MeOH); Flow rate: 140 mL/min; Gradient: isocratic 50% B; RT1(min): 7; RT2(min): 10; Sample Solvent: MeOH; Injection Volume: 1.5 mL; number Of Runs: 300). The fractions of faster eluting peak was collected and concentrated under vacuum to afford (R)-6-chloro-N-(2,2-difluorocyclobutyl)-8-((4-methoxybenzyl)(methyl)amino)imidazo[1,2-b]pyridazine-3-carboxamide (2.3 g) as a brown solid. LC/MS (ESI, m/z): [(M+1)]⁺=436.2. The fractions of slower eluting peak was collected and concentrated under vacuum to afford (S)-6-chloro-N-(2,2-difluorocyclobutyl)-8-((4-methoxybenzyl)(methyl)amino)imidazo[1,2-b]pyridazine-3-carboxamide (1.8 g) as a brown solid. LC/MS (ESI, m/z): [(M+1)]⁺=436.2. The absolute stereochemistry of the enantiomers was assigned arbitrarily.

### (R)—N-(2,2-difluorocyclobutyl)-6-(4-(3-fluoro-5-formylpyridin-2-yl)indolin-1-yl)-8-(methylamino)imidazo[1,2-b]pyridazine-3-carboxamide 2,2,2-trifluoroacetate (Intermediate NW)

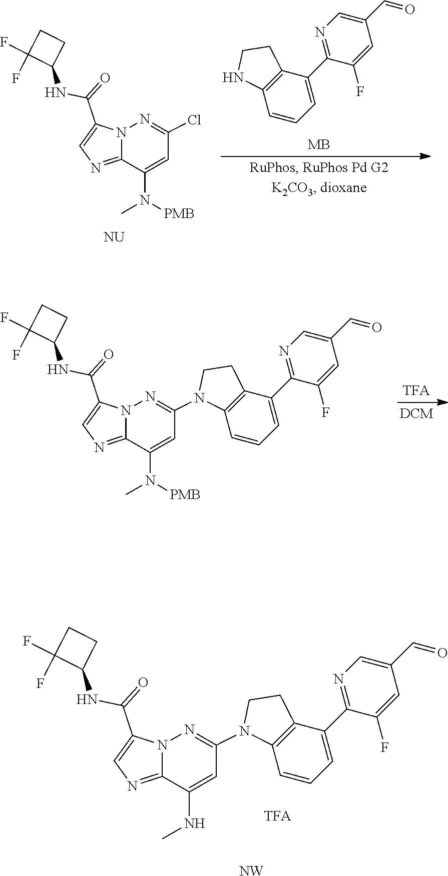

Step 1—(R)—N-(2,2-difluorocyclobutyl)-6-(4-(3-fluoro-5-formylpyridin-2-yl)indolin-1-yl)-8-((4-methoxybenzyl)(methyl)amino)imidazo[1,2-b]pyridazine-3-carboxamide. To a stirred mixture of (R)-6-chloro-N-(2,2-difluorocyclobutyl)-8-((4-methoxybenzyl)(methyl)amino)imidazo[1,2-b]pyridazine-3-carboxamide (500 mg, 1.15 mmol, Intermediate NU) and 5-fluoro-6-(indolin-4-yl)nicotinaldehyde (277 mg, 1.14 mmol, Intermediate MB) in 1,4-dioxane (5 mL) were added K₂CO₃ (475 mg, 3.44 mmol), RuPhos (107 mg, 0.229 mmol) and RuPhos-PdCl-2nd G (178 mg, 0.229 mmol) at rt under nitrogen atmosphere. The resulting mixture was stirred for 2 h at 100° C. under nitrogen atmosphere. On completion, the solution was cooled to rt and purified by reverse phase flash chromatography (Column: WelFlash™ C18-I, 20-40 μm, 330 g; Eluent A: Water (plus 10 mmol/L NH₄HCO₃); Eluent B: ACN; Gradient: 45%-65% B in 25 min; Flow rate: 80 mL/min; Detector: 220/254 nm; desired fractions were collected at 54% B) and concentrated under reduced pressure to give the title compound (600 mg, 81% yield) as a white solid. LC/MS (ESI, m/z): [(M+1)]⁺=642.2.

Step 2—(R)—N-(2,2-difluorocyclobutyl)-6-(4-(3-fluoro-5-formylpyridin-2-yl)indolin-1-yl)-8-(methylamino)imidazo[1,2-b]pyridazine-3-carboxamide 2,2,2-trifluoroacetate. To a stirred solution of (R)—N-(2,2-difluorocyclobutyl)-6-(4-(3-fluoro-5-formylpyridin-2-yl)indolin-1-yl)-8-((4-methoxybenzyl)(methyl)amino)imidazo[1,2-b]pyridazine-3-carboxamide (600 mg, 0.936 mmol) in DCM (12 mL) was added TFA (4 mL) at rt and the mixture was stirred for 1 h at rt. On completion, the mixture was concentrated under reduced pressure. The residue was purified by trituration with ethyl ether (20 mL). The precipitated solids were collected hv filtration and washed with ethyl ether (2×5 mL). The resulting mixture was concentrated under reduced pressure to give the title compound (650 mg, TFA salt) as a brown solid. ¹H NMR (400 MHz, DMSO-d₆) δ 10.19 (d, J=1.6 Hz, 1H), 9.13-9.03 (m, 2H), 8.28 (dd. J=10.4, 1.6 Hz, 1H), 7.99 (s, 1H), 7.91 (d, J=8.0 Hz, 1H), 7.80-7.69 (m, 1H), 7.35 (t, J=8.0 Hz, 1H), 7.24-7.14 (m, 1H), 6.01 (s, 1H), 5.12-4.98 (m, 1H), 4.34-4.13 (m, 2H), 3.26 (t, J=8.4 Hz, 2H), 2.97 (d, J=4.8 Hz, 3H), 2.48-2.37 (m, 2H), 2.35-2.24 (m, 1H), 1.76-1.63 (m, 1H). LC/MS (ESI, m/z): [(M+1)]+=522.2.

### (R)-6-(4-(5-((5,5-difluoro-2,7-diazaspiro[3.5]nonan-2-yl)methyl)-3-fluoropyridin-2-yl)indolin-1-yl)-N-(2,2-difluorocyclobutyl)-8-(methylamino)imidazo[1,2-b]pyridazine-3-carboxamide 2,2,2-trifluoroacetate (Intermediate NX)

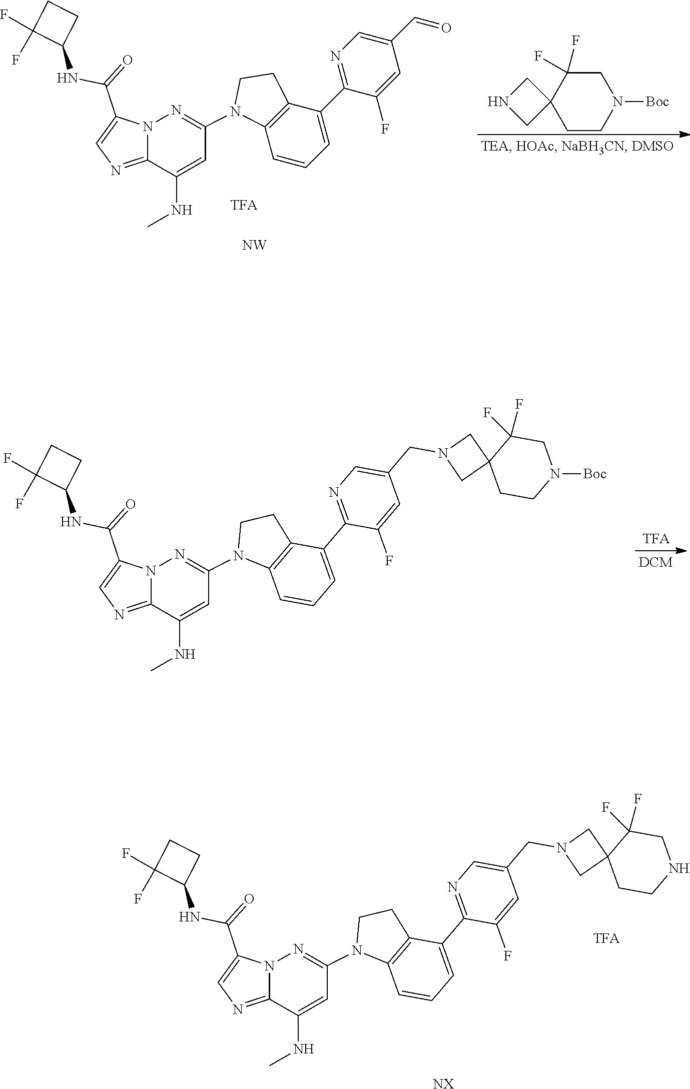

Step 1—Tert-butyl (R)-2-((6-(1-(3-((2,2-difluorocyclobutyl)carbamoyl)-8-(methylamino)imidazo[1,2-b]pyridazin-6-yl)indolin-4-yl)-5-fluoropyridin-3-yl)methyl)-5,5-difluoro-2,7-diazaspiro[3.5]nonane-7-carboxylate. To a stirred solution of (R)—N-(2,2-difluorocyclobutyl)-6-(4-(3-fluoro-5-formylpyridin-2-yl)indolin-1-yl)-8-(methylamino)imidazo[1,2-b]pyridazine-3-carboxamide 2,2,2-trifluoroacetate (200 mg, 0.384 mmol, Intermediate NW) and TEA (38 mg, 0.376 mmol) in DMSO (2 mL) were added tert-butyl 5,5-difluoro-2,7-diazaspiro[3.5]nonane-7-carboxylate (100 mg, 0.381 mmol) and HOAc (69 mg, 1.149 mmol) at rt. The resulting mixture was stirred for 1 h at 50° C. To the above mixture was added NaBH₃CN (96 mg, 1.528 mmol) at rt. The resulting mixture was stirred for an additional 1 h at 50° C. On completion, the solution was cooled to rt and purified by reverse phase flash chromatography (Column: WelFlash™ C18-I, 20-40 μm, 330 g; Eluent A: Water (plus 10 mmol/L NH4HCO₃); Eluent B: ACN; Gradient: 45%-85% B in 25 min; Flow rate: 60 mL/min; Detector: 220/254 nm; desired fractions were collected at 70% B) and concentrated under reduced pressure to give the title compound (140 mg, 47% yield) as a white solid. LC/MS (ESI, m/z): [(M+1)]⁺=768.3.

Step 2—(R)-6-(4-(5-((5,5-difluoro-2,7-diazaspiro[3.5]nonan-2-yl)methyl)-3-fluoropyridin-2-yl)indolin-1-yl)-N-(2,2-difluorocyclobutyl)-8-(methylamino)imidazo[1,2-b]pyridazine-3-carboxamide 2,2,2-trifluoroacetate. To a stirred solution of tert-butyl (R)-2-((6-(1-(3-((2,2-difluorocyclobutyl)carbamoyl)-8-(methylamino)imidazo[1,2-b]pyridazin-6-yl)indolin-4-yl)-5-fluoropyridin-3-yl)methyl)-5,5-difluoro-2,7-diazaspiro[3.5]nonane-7-carboxylate (140 mg, 0.182 mmol) in DCM (2 mL) was added TFA (0.5 mL) at rt and the mixture was stirred for 1 h at rte. The resulting mixture was concentrated under reduced pressure. The residue was purified by trituration with ethyl ether (5 mL). The precipitated solids were collected by filtration and washed with ethyl ether (2×2 mL). The resulting solid was dried under reduced pressure to give the title compound (170 mg, TFA salt) as a brown solid. LC/MS (ESI, m/z): [(M+1)]⁺=668.3.

### 6-(4-(3-fluoro-5-formylpyridin-2-yl)indolin-1-yl)-N-((1R,2R)-2-fluorocyclobutyl)-8-(methylamino)imidazo[1,2-b]pyridazine-3-carboxamide trifluoroacetate (Intermediate NY)

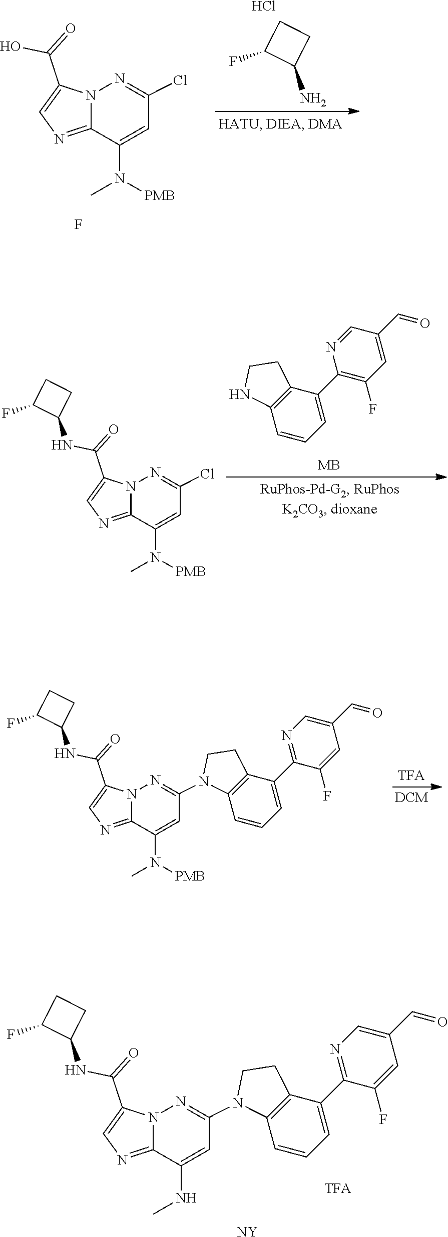

Step 1—6-Chloro-N-((1R,2R)-2-fluorocyclobutyl)-8-((4-methoxybenzyl)(methyl)amino)imidazo[1,2-b]pyridazine-3-carboxamide. A solution of 6-chloro-8-((4-methoxybenzyl)(methyl)amino)imidazo[1,2-b]pyridazine-3-carboxylic acid (1.37 g, 3.94 mmol, Intermediate F) and HATU (1.64 g, 4.30 mmol) in DMA (10 mL) was treated with DIEA (1.39 g, 10.75 mmol) for 10 min at rt followed by the addition of (1R,2R)-2-fluorocyclobutan-1-amine hydrochloride (450 mg, 3.584 mmol, CAS #2920233-59-4) at rt. The resulting mixture was stirred for 1 h at rt. On completion, the mixture was purified by reversed-phase flash chromatography (Column: Spherical C18, 20˜40 m, 330 g; Mobile Phase A: Water(plus 10 mM NH₄HCO₃), Mobile Phase B: ACN; Flow rate: 80 mL/min; Gradient (B %): 5%˜50%, 4 min; 50%-70%, 20 min; 70%-95%; 2 min; 95%, 5 min; Detector: 254 nm; the fractions containing the desired product were collected at 69% B) and concentrated under reduced pressure to afford the title compound (1.4 g, 93% yield) as a white solid. LC/MS (ESI, m/z): [(M+1)]⁺=418.1.

Step 2—6-(4-(3-Fluoro-5-formylpyridin-2-yl)indolin-1-yl)-N-((1R,2R)-2-fluorocyclobutyl)-8-((4-methoxybenzyl)(methyl)amino)imidazo[1,2-b]pyridazine-3-carboxamide. To a stirred mixture of 6-chloro-N-((1R,2R)-2-fluorocyclobutyl)-8-((4-methoxybenzyl)(methyl)amino)imidazo[1,2-b]pyridazine-3-carboxamide (318 mg, 1.32 mmol), 5-fluoro-6-(indolin-4-yl)nicotinaldehyde (500 mg, 1.20 mmol, Intermediate MB) and K₂CO₃ (496 mg, 3.59 mmol) in dioxane (5 mL) were added RuPhos (55 mg, 0.12 mmol) and RuPhos-PdCl-2nd G (93 mg, 0.12 mmol) at rt under nitrogen atmosphere. The resulting mixture was stirred for 1 h at 100° C. under nitrogen atmosphere. On completion, the mixture was cooled to rt and concentrated under reduced pressure. The resulting mixture was purified by reversed-phase flash chromatography (Column: Spherical C18, 20˜40 m, 330 g; Mobile Phase A: Water(plus 10 mM NH4HCO₃), Mobile Phase B: ACN; Flow rate: 80 mL/min; Gradient (B %): 5%˜70%, 4 min; 70%˜95%, 20 min; 95%˜95%; 2 min; 95%, 5 min; Detector: 254 nm; the fractions containing desired product were collected at 80% B) and concentrated under reduced pressure to afford the title compound (650 mg, 87% yield) as a white solid: LC/MS (ESI, m/z): [(M+1)]⁺=624.2.

Step 3—6-(4-(3-Fluoro-5-formylpyridin-2-yl)indolin-1-yl)-N-((1R,2R)-2-fluorocyclobutyl)-8-(methylamino)imidazo[1,2-b]pyridazine-3-carboxamide trifluoroacetate. To a stirred mixture of 6-(4-(3-fluoro-5-formylpyridin-2-yl)indolin-1-yl)-N-((1R,2R)-2-fluorocyclobutyl)-8-((4-methoxybenzyl)(methyl)amino)imidazo[1,2-b]pyridazine-3-carboxamide (650 mg, 1.04 mmol) in DCM (6 mL) was added TFA (2 mL) dropwise at rt and the resulting mixture was stirred for 1 h at rt. On completion, the reaction was washed with Et₂O (2×10 mL) to afford the title compound (620 mg, 98% yield) as a yellow solid. LC/MS (ESI, m/z): [(M+1)]⁺=504.3.

### 6-(4-(5-((5,5-Difluoro-2,7-diazaspiro[3.5]nonan-2-yl)methyl)-3-fluoropyridin-2-yl)indolin-1-yl)-N-((1R,2R)-2-fluorocyclobutyl)-8-(methylamino)imidazo[1,2-b]pyridazine-3-carboxamide trifluoroacetate (Intermediate NZ)

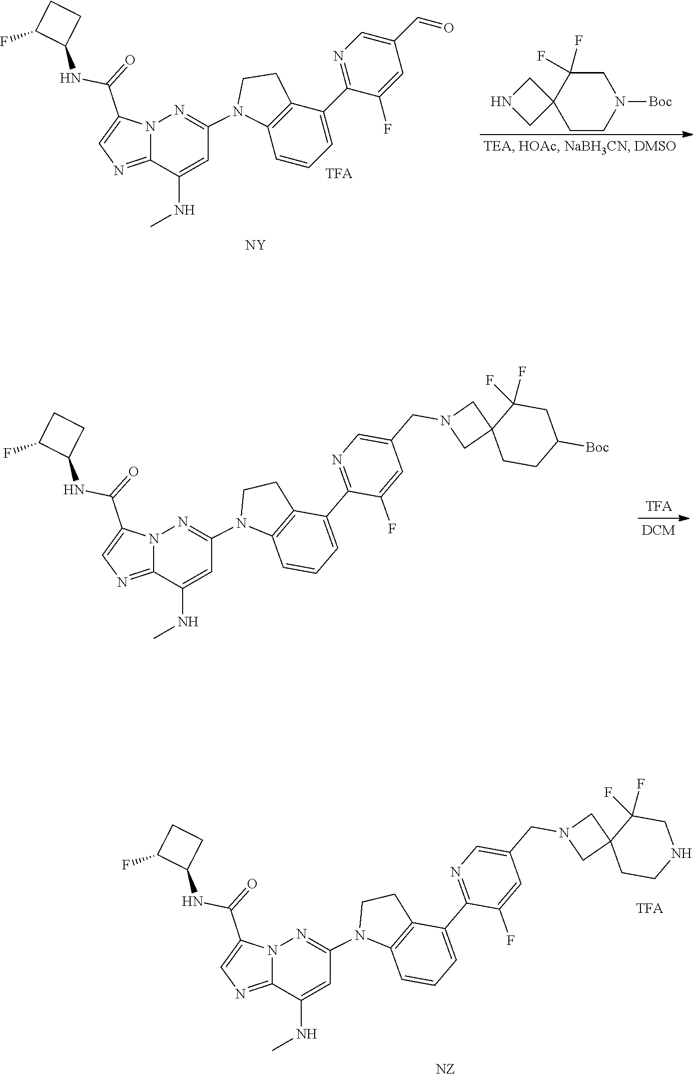 [SMILES: CNc1cc(N2CCc3c(-c4ncc(C=O)cc4F)cccc32)nn2c(C(=O)N[C@@H]3CC[C@H]3F)cnc12 ; CC(C)(C)OC(=O)N1CCC2(CNC2)C(F)(F)C1 ; "NY" ; CNc1cc(N2CCc3c(-c4ncc(CN5CC6(CCC(C(=O)OC(C)(C)C)CC6(F)F)C5)cc4F)cccc32)nn2c(C(=O)N[C@@H]3CC[C@H]3F)cnc12 ; CNc1cc(N2CCc3c(-c4ncc(CN5CC6(CCNCC6(F)F)C5)cc4F)cccc32)nn2c(C(=O)N[C@@H]3CC[C@H]3F)cnc12 ; "NZ"]

Step 1—Tert-butyl 5,5-difluoro-2-((5-fluoro-6-(1-(3-(((1R,2R)-2-fluorocyclobutyl)carbamoyl)-8-(methylamino)imidazo[1,2-b]pyridazin-6-yl)indolin-4-yl)pyridin-3-yl)methyl)-2,7-diazaspiro[3.5]nonane-7-carboxylate. To a stirred mixture of 6-(4-(3-fluoro-5-formylpyridin-2-yl)indolin-1-yl)-N-((1R,2R)-2-fluorocyclobutyl)-8-(methylamino)imidazo[1,2-b]pyridazine-3-carboxamide trifluoroacetate (1.00 g, 1.62 mmol, Intermediate NY) and tert-butyl 5,5-difluoro-2,7-diazaspiro[3.5]nonane-7-carboxylate (425 mg, 1.62 mmol) in DMSO (8 mL) were added Et₃N (327 mg, 3.24 mmol) and HOAc (973 mg, 16.2 mmol) at rt. The resulting mixture was stirred for 1 h at 50° C. To the above mixture was added NaBH₃CN (511 mg, 8.105 mmol) in portions at rt and the resulting mixture was stirred for an additional 1 h at 50° C. On completion, the mixture was cooled to rt and purified by reversed-phase flash chromatography (Column: Spherical C18, 20-40 m, 330 g; Mobile Phase A: Water(plus 10 mM NH₄HCO₃), Mobile Phase B: ACN; Flow rate: 80 mL/min; Gradient (B %): 5%˜65%, 4 min; 65%˜80%, 20 min; 80%˜95%; 5 min; Detector: 254 nm; the fractions containing desired product were collected at 77% B) and concentrated under reduced pressure to afford the title compound (650 mg, 45% yield) as a white solid: LC/MS (ESI, m/z): [(M+1)]+=750.4.

Step 2—6-(4-(5-((5,5-Difluoro-2,7-diazaspiro[3.5]nonan-2-yl)methyl)-3-fluoropyridin-2-yl)indolin-1-yl)-N-((1R,2R)-2-fluorocyclobutyl)-8-(methylamino)imidazo[1,2-b]pyridazine-3-carboxamide trifluoroacetate. To a stirred mixture of tert-butyl 5,5-difluoro-2-((5-fluoro-6-(1-(3-(((1R,2R)-2-fluorocyclobutyl)carbamoyl)-8-(methylamino)imidazo[1,2-b]pyridazin-6-yl)indolin-4-yl)pyridin-3-yl)methyl)-2,7-diazaspiro[3.5]nonane-7-carboxylate (650 mg, 0.868 mmol) in DCM (12 mL) was added TFA (4 mL) dropwise at rt and the mixture was stirred for 1 h at rt. On completion, the mixture was washed with Et₂O (2×20 mL) and concentrated under reduced pressure to afford the title compound (650 mg, TFA salt) as a yellow solid. LC/MS (ESI, m/z): [(M+1)]⁺=650.2.

### (R)—N-(3-(5-((1,1-difluoro-3,9-diazaspiro[5.5]undecan-3-yl)methyl)-6′-(3-methoxytetrahydrofuran-3-yl)-[2,4′-bipyridin]-2′-yl)-1-methyl-1H-pyrrolo[2,3-c]pyridin-5-yl)acetamide (Intermediate OA)

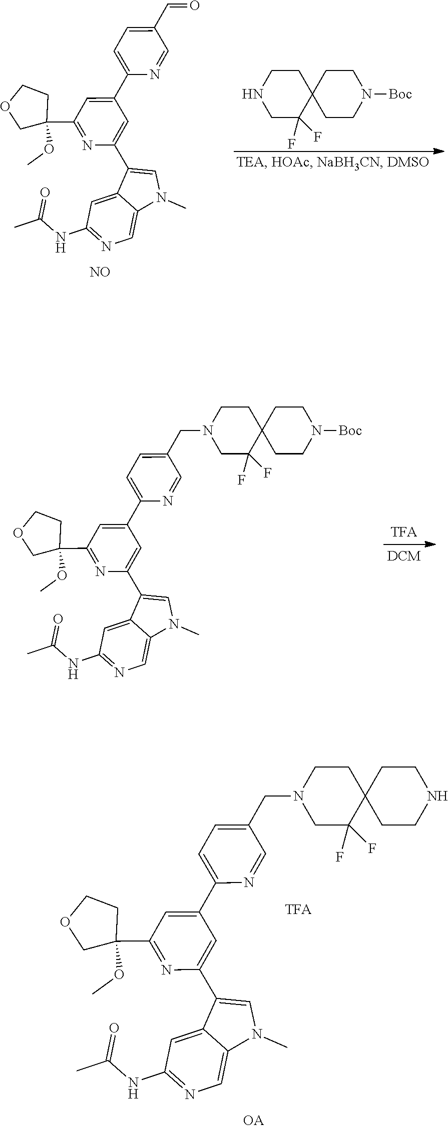 [SMILES: CO[C@@]1(c2cc(-c3ccc(C=O)cn3)cc(-c3cn(C)c4cnc(NC(C)=O)cc34)n2)CCOC1 ; CC(C)(C)OC(=O)N1CCC2(CCNCC2(F)F)CC1 ; "NO" ; CO[C@@]1(c2cc(-c3ccc(CN4CCC5(CCN(C(=O)OC(C)(C)C)CC5)C(F)(F)C4)cn3)cc(-c3cn(C)c4cnc(NC(C)=O)cc34)n2)CCOC1 ; CO[C@@]1(c2cc(-c3ccc(CN4CCC5(CCNCC5)C(F)(F)C4)cn3)cc(-c3cn(C)c4cnc(NC(C)=O)cc34)n2)CCOC1]

Step 1—Tert-butyl 9-[(2′-{5-acetamido-1-methylpyrrolo[2,3-c]pyridin-3-yl}-6′-[(3R)-3-methoxyoxolan-3-yl]-[2,4′-bipyridin]-5-yl)methyl]-7,7-difluoro-3,9-diazaspiro[5.5]undecane-3-carboxylate. To a stirred solution of N-(3-{5-formyl-6′-[(3R)-3-methoxyoxolan-3-yl]-[2,4′-bipyridin]-2′-yl}-1-methylpyrrolo[2,3-c]pyridin-5-yl)acetamide (90 mg, 0.192 mmol, Intermediate NO) in DMSO (1 mL) was added TEA (0.03 mL, 0.192 mmol) at rt under nitrogen atmosphere. To the above mixture were added tert-butyl 7,7-difluoro-3,9-diazaspiro[5.5]undecane-3-carboxylate (18.47 mg, 0.064 mmol, CAS #1784848-04-9) and AcOH (0.01 mL, 0.192 mmol) at rt. The resulting mixture was stirred for additional 0.5 h at 50° C. The mixture was allowed to cool down to room temperature. Next, NaBH₃CN (8.00 mg, 0.128 mmol) was added at rt. The resulting mixture was stirred for an additional 16 h at 50° C. On completion, the mixture was cooled to rt and concentrated under vacuum. The residue was purified by reverse phase flash chromatography (Column: WelFlash™ C18-I, 20-40 μm, 120 g; Eluent A: Water (plus 10 mmol/L NH4HCO₃); Eluent B: ACN; Gradient: 35%-65% B in 25 min; Flow rate: 80 mL/min; Detector: 220/254 nm; desired fractions were collected at 60% B) and concentrated under reduced pressure to afford the title compound (17 mg, 36% yield) as a yellow solid. LC/MS (ESI, m/z): [(M+H)]⁺=746.3.

Step 2—N-{3-[5-({1,1-difluoro-3,9-diazaspiro[5.5]undecan-3-yl}methyl)-6′-[(3R)-3-methoxyoxolan-3-yl]-[2,4′-bipyridin]-2′-yl]-1-methylpyrrolo[2,3-c]pyridin-5-yl}acetamide trifluoroacetate. To a stirred solution of tert-butyl 9-[(2′-{5-acetamido-1-methylpyrrolo[2,3-c]pyridin-3-yl}-6′-[(3R)-3-methoxyoxolan-3-yl]-[2,4′-bipyridin]-5-yl)methyl]-7,7-difluoro-3,9-diazaspiro[5.5]undecane-3-carboxylate (17 mg, 0.023 mmol) in DCM (1 mL) was added TFA (0.5 mL) at rt under nitrogen atmosphere. The resulting mixture was stirred for 30 min at rt under nitrogen atmosphere. On completion, the mixture was concentrated under vacuum. The residue was purified by trituration with Et₂O (10 mL) to the title compound (16 mg, 94% yield) as a brown solid. LC/MS (ESI, m/z): [(M+H)]=646.3.

### 3-(2,4-dioxotetrahydropyrimidin-1(2H)-yl)-4,5-dimethylbenzoic acid (Intermediate OB)

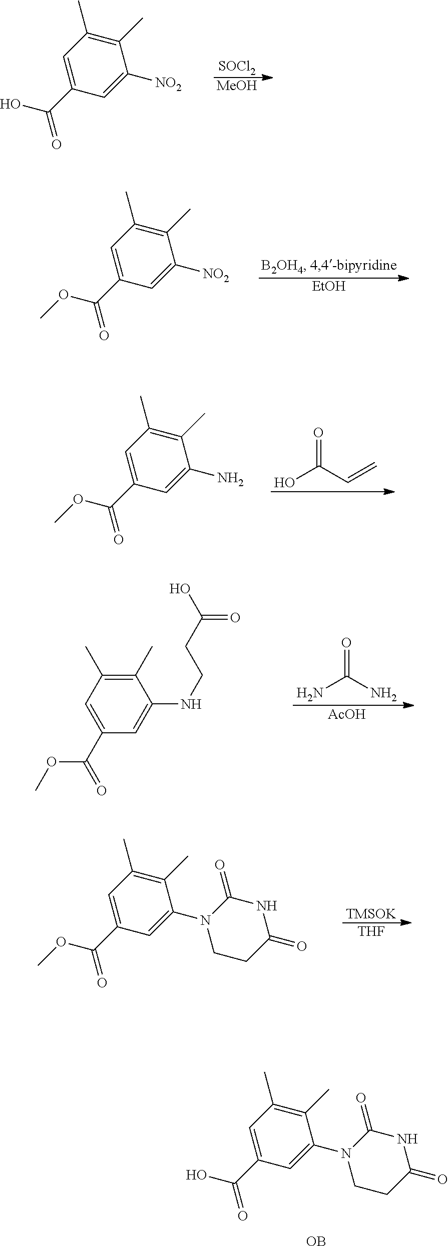

Step 1—Methyl 3,4-dimethyl-5-nitrobenzoate. To a stirred solution of 3,4-dimethyl-5-nitrobenzoic acid (4.00 g, 20.5 mmol, CAS #74319-96-3) in MeOH (60 mL) was added SOCl₂ (12.19 g, 102.4 mmol) dropwise at rt under nitrogen atmosphere. The resulting mixture was stirred for 1 h at 70° C. under nitrogen atmosphere. On completion, the mixture was cooled to rt and concentrated under vacuum. The mixture was basified to pH 7 with saturated NaHCO₃ (aq.). The resulting mixture was extracted with EtOAc (2×300 mL). The combined organic layers were washed with brine (2×100 mL), dried over anhydrous Na₂SO₄, filtered, and the filtrate was concentrated under reduced pressure to give the title compound (3.90 g, 90% yield) as a yellow oil. ¹H NMR (300 MHz, DMSO-d₆) δ 8.17-8.11 (m, 1H), 8.07-8.00 (m, 1H), 3.89 (s, 3H), 2.42 (s, 3H), 2.36 (s, 3H).

Step 2—Methyl 3-amino-4,5-dimethylbenzoate. To a stirred solution of methyl 3,4-dimethyl-5-nitrobenzoate (10.00 g, 47.80 mmol) and B₂(OH)₄ (6.43 g, 71.70 mmol) in EtOH (150 mL) was added 4-(pyridin-4-yl)pyridine (0.75 g, 4.78 mmol) in portions at rt under nitrogen atmosphere. The resulting mixture was stirred for 1 h at rt under nitrogen atmosphere. On completion, the mixture was extracted with EtOAc (3×300 mL). The combined organic layers were washed with brine (2×300 mL), dried over anhydrous Na₂SO₄, filtered, and the filtrate was concentrated under reduced pressure to give the title compound (8.00 g, 93% yield) as a yellow oil. LC/MS (ESI, m/z): [(M+1)]⁺=180.1.

Step 3—3-((5-(Methoxycarbonyl)-2,3-dimethylphenyl)amino)propanoic acid. A stirred solution of methyl 3-amino-4,5-dimethylbenzoate (10.00 g, 55.79 mmol) in acrylic acid (8.04 g, 112 mmol) was stirred for 1 h at 100° C. under nitrogen atmosphere. On completion, the mixture was cooled to rt and extracted with EtOAc (3×300 mL). The combined organic layers were washed with brine (2×300 mL), dried over anhydrous Na₂SO₄, filtered, and the filtrate was concentrated under reduced pressure to give the title compound (14.00 g, 99% yield) as a yellow oil. LC/MS (ESI, m/z): [(M+1)]⁺=252.1.

Step 4—Methyl 3-(2,4-dioxotetrahydropyrimidin-1(2H)-yl)-4,5-dimethylbenzoate. To a stirred solution of 3-((5-(methoxycarbonyl)-2,3-dimethylphenyl)amino)propanoic acid (12.00 g, 47.75 mmol) in CH₃COOH (150 mL) was added urea (20.08 g, 334.3 mmol) in portions at rt under nitrogen atmosphere. The resulting mixture was stirred for 5 h at 110° C. under nitrogen atmosphere. On completion, the mixture was cooled to rt and concentrated under reduced pressure. The residue was purified by silica gel column chromatography, eluted with PE/EA (1:1), to afford the title compound (5.20 g, 39% yield) as a white solid. LC/MS (ESI, m/z): [(M+1)]⁺=277.2.

Step 5—3-(2,4-Dioxotetrahydropyrimidin-1(2H)-yl)-4,5-dimethylbenzoic acid. To a stirred solution of methyl 3-(2,4-dioxotetrahydropyrimidin-1(2H)-yl)-4,5-dimethylbenzoate (4.00 g, 14.5 mmol) in THF (60 mL) was added potassium trimethylsilanolate (9.29 g, 72.4 mmol) in portions at rt under nitrogen atmosphere. The resulting mixture was stirred for 1 h at rt under nitrogen atmosphere. On completion, the mixture was acidified to pH 3 with cone. HCl. The precipitated solids were collected by filtration and washed with H₂O (3×100 mL) to give the title compound (2.30 g, 60% yield) as a yellow solid. LC/MS (ESI, m/z): [(M+1)]⁺=263.3. 6-Chloro-N-cyclobutyl-8-((4-methoxybenzyl)(methyl)amino)imidazo[1,2-b]pyridazine-3-carboxamide (Intermediate OC)

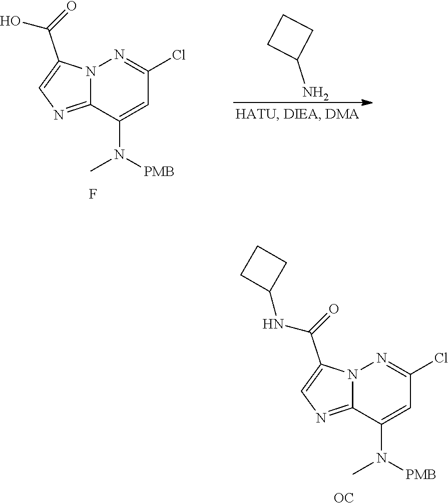

To a stirred mixture of 6-chloro-8-{[(4-methoxyphenyl)methyl](methyl)amino}imidazo[1,2-b]pyridazine-3-carboxylic acid (500 mg, 1.44 mmol, Intermediate F) and HATU (822.38 mg, 2.163 mmol) in DMA (5 mL) was added DIEA (559.08 mg, 4.326 mmol) in portions at rt under nitrogen atmosphere and the mixture was stirred for 5 min at rt under nitrogen atmosphere. To the above mixture was added cyclobutylamine (102.55 mg, 1.442 mmol) in portions at rt and the mixture was stirred for additional 1 h at rt. The reaction mixture was purified directly by reverse phase flash chromatography (Column: WelFlash™ C18-I, 20-40 μm, 330 g; Eluent A: Water (plus 10 mmol/L NH4HCO3); Eluent B: ACN; Gradient: 25%-55% B in 25 min; Flow rate: 80 mL/min; Detector: 220/254 nm; desired fractions were collected at 70% B) and concentrated under reduced pressure to afford the title compound (500 mg, 87% yield) as a yellow solid. LC/MS (ESI, m/z): [(M+1)]⁺=400.2.

### N-cyclobutyl-6-(4-(3-fluoro-5-formylpyridin-2-yl)indolin-1-yl)-8-(methylamino)imidazo[1,2-b]pyridazine-3-carboxamide (Intermediate OD)

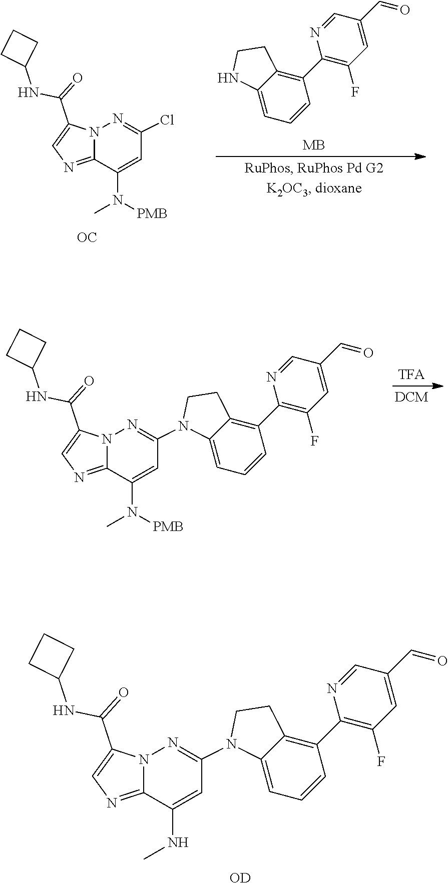

Step 1—N-cyclobutyl-6-(4-(3-fluoro-5-formylpyridin-2-yl)indolin-1-yl)-8-((4-methoxybenzyl)(methyl)amino)imidazo[1,2-b]pyridazine-3-carboxamide. To a stirred mixture of 6-chloro-N-cyclobutyl-8-{[(4-methoxyphenyl)methyl](methyl)amino}imidazo[1,2-b]pyridazine-3-carboxamide (500 mg, 1.25 mmol, Intermediate OC) and 6-(2,3-dihydro-1H-indol-4-yl)-5-fluoropyridine-3-carbaldehyde (302.91 mg, 1.250 mmol, Intermediate MB) in 1,4-dioxane (5 mL) was added K₂CO₃ (518.42 mg, 3.750 mmol), RuPhos-PdCl-2nd G (97.25 mg, 0.125 mmol), and RuPhos (116.70 mg, 0.250 mmol) in turns at rt under nitrogen atmosphere. The resulting mixture was stirred for 1 h at 100° C. under nitrogen atmosphere. On completion, the mixture was cooled to rt and concentrated under vacuum. The residue was purified by silica gel column chromatography, eluted with PE/EA (1:1), to afford the title compound (570 mg, 75% yield) as a yellow solid. LC/MS (ESI, m/z): [(M+1)]⁺=606.3.

Step 2—N-cyclobutyl-6-(4-(3-fluoro-5-formylpyridin-2-yl)indolin-1-yl)-8-(methylamino)imidazo[1,2-b]pyridazine-3-carboxamide. To a stirred mixture of N-cyclobutyl-6-[4-(3-fluoro-5-formylpyridin-2-yl)-2,3-dihydroindol-1-yl]-8-{[(4-methoxyphenyl)methyl](methyl)amino}imidazo[1,2-b]pyridazine-3-carboxamide (570 mg, 0.941 mmol) in DCM (6 mL) was added TFA (2 mL) dropwise at rt under nitrogen atmosphere. The resulting mixture was stirred for 1 h at rt under nitrogen atmosphere. On completion, the mixture was concentrated under reduced pressure. The residue was purified by trituration with ethyl ether (50 mL) to give the title compound (450 mg, 99% yield) as a yellow solid. LC/MS (ESI, m/z): [(M+1)]⁺=486.2.

### N-cyclobutyl-6-(4-(5-((5,5-difluoro-2,7-diazaspiro[3.5]nonan-2-yl)methyl)-3-fluoropyridin-2-yl)indolin-1-yl)-8-(methylamino)imidazo[1,2-b]pyridazine-3-carboxamide trifluoroacetate (Intermediate OE)

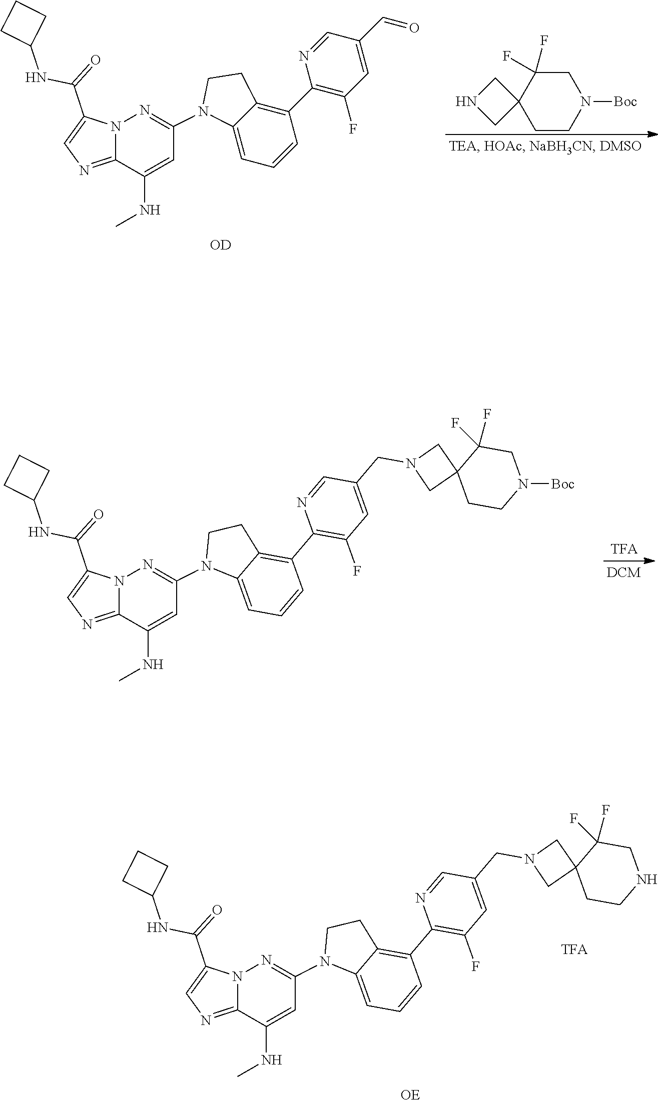

Step 1—Tert-butyl 2-((6-(1-(3-(cyclobutylcarbamoyl)-8-(methylamino)imidazo[1,2-b]pyridazin-6-yl)indolin-4-yl)-5-fluoropyridin-3-yl)methyl)-5,5-difluoro-2,7-diazaspiro[3.5]nonane-7-carboxylate. To a stirred mixture of tert-butyl 5,5-difluoro-2,7-diazaspiro[3.5]nonane-7-carboxylate (108.05 mg, 0.412 mmol) and TEA (41.68 mg, 0.412 mmol) in DMSO (2 mL) was added N-cyclobutyl-6-[4-(3-fluoro-5-formylpyridin-2-yl)-2,3-dihydroindol-1-yl]-8-(methylamino)imidazo[1,2-b]pyridazine-3-carboxamide (200 mg, 0.412 mmol, Intermediate OD), and HOAc (98.95 mg, 1.648 mmol) in portions at rt under nitrogen atmosphere. The resulting mixture was stirred for 1 h at 50° C. under nitrogen atmosphere. The mixture was allowed to cool down to room temperature. To the above mixture was added NaBH₃CN (129.43 mg, 2.060 mmol) in portions at rt. The resulting mixture was stirred for an additional 1 h at 50° C. On completion, the mixture was cooled to rt and purified by reverse phase flash chromatography (Column: WelFlash™ C18-I, 20-40 μm, 120 g; Eluent A: Water (plus 10 mmol/L NH₄HCO₃); Eluent B: ACN: Gradient: 60%-90% B in 25 min; Flow rate: 80 mL/min; Detector: 220/254 nm; desired fractions were collected at 75% B) and concentrated under reduced pressure to afford the title compound (120 mg, 40% yield) as a yellow solid. LC/MS (ESI, m/z): [(M+1)]⁺=732.4.

Step 2—N-cyclobutyl-6-(4-(5-((5,5-difluoro-2,7-diazaspiro[3.5]nonan-2-yl)methyl)-3-fluoropyridin-2-yl)indolin-1-yl)-8-(methylamino)imidazo[1,2-b]pyridazine-3-carboxamide trifluoroacetate. To a stirred mixture of tert-butyl 2-[(6-{1-[3-(cyclobutylcarbamoyl)-8-(methylamino)imidazo[1,2-b]pyridazin-6-yl]-2,3-dihydroindol-4-yl}-5-fluoropyridin-3-yl)methyl]-5,5-difluoro-2,7-diazaspiro[3.5]nonane-7-carboxylate (120 mg, 0.164 mmol) in DCM (3 mL) was added TFA (1 mL) dropwise at rt under nitrogen atmosphere and the mixture was stirred for 1 h at rt under nitrogen atmosphere. On completion, the mixture was concentrated under reduced pressure. The residue was purified by trituration with ethyl ether (10 mL) to afford the title compound (100 mg, 97% yield) as a yellow solid. LC/MS (ESI, m/z): [(M+1)]⁺=632.3.

### 6-Chloro-N-cyclopropyl-8-((4-methoxybenzyl)(methyl)amino)imidazo[1,2-b]pyridazine-3-carboxamide (Intermediate OF)

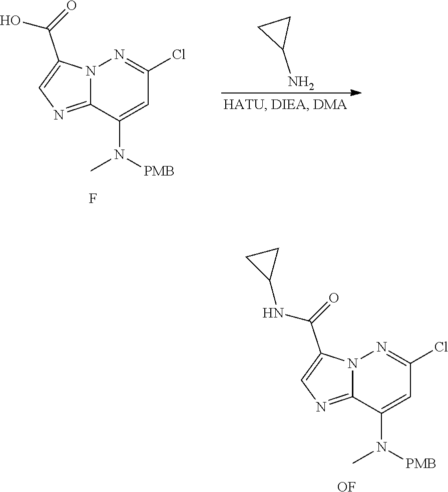

To a stirred solution of 6-chloro-8-((4-methoxybenzyl)(methyl)amino)imidazo[1,2-b]pyridazine-3-carboxylic acid (500 mg, 1.44 mmol, Intermediate F), HATU (822 mg, 2.16 mmol) and DIEA (559 mg, 4.33 mmol) in DMA (10 mL) at it and the mixture was stirred for 3 min at rt. Then cyclopropanamine (82 mg, 1.442 mmol) was added at rt and the mixture was stirred for 1 h at rt. After completion of the reaction, the reaction mixture was purified directly by reverse phase flash chromatography (Column: WelFlash™ C18-I, 20-40 m, 330 g; Eluent A: Water (plus 10 mmol/L NH4HCO3); Eluent B: ACN; Gradient: 50% -95% B in 30 min; Flow rate: 80 mL/min; Detector: 220/254 nm; desired fractions were collected at 78% B) and concentrated under reduced pressure to afford the title compound (460 mg, 82% yield) as a yellow solid. LC/MS (ESI, m/z): [(M+1)]⁺=386.3.

### N-cyclopropyl-6-(4-(3-fluoro-5-formylpyridin-2-yl)indolin-1-yl)-8-(methylamino)imidazo[1,2-b]pyridazine-3-carboxamide trifluoroacetate (Intermediate OG)

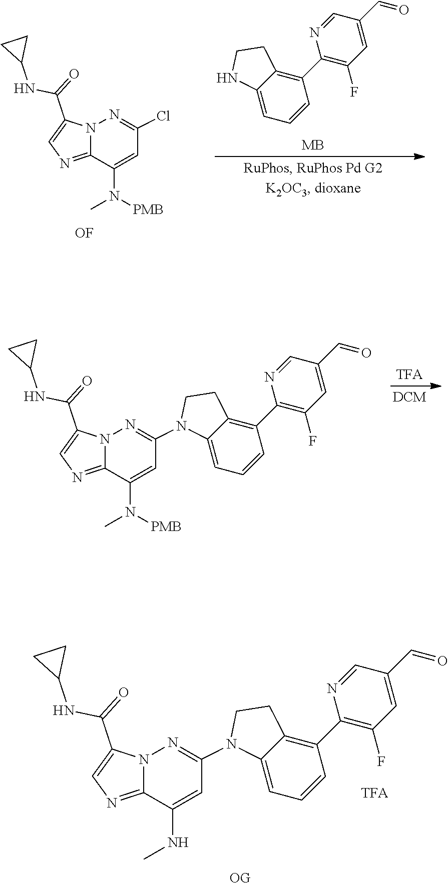

Step 1—N-cyclopropyl-6-(4-(3-fluoro-5-formylpyridin-2-yl)indolin-1-yl)-8-((4-methoxybenzyl)(methyl)amino)imidazo[1,2-b]pyridazine-3-carboxamide. To a stirred solution of 6-chloro-N-cyclopropyl-8-((4-methoxybenzyl)(methyl)amino)imidazo[1,2-b]pyridazine-3-carboxamide (460 mg, 1.19 mmol, Intermediate OF), K₂CO₃ (494 mg, 3.576 mmol) and 5-fluoro-6-(indolin-4-yl)nicotinaldehyde (346 mg, 1.43 mmol, Intermediate MB) in 1,4-dioxane (10 mL) were added RuPhos (111 mg, 0.238 mmol) and RuPhos-PdCl-2nd G (93 mg, 0.12 mmol) at rt under nitrogen atmosphere. The resulting mixture was stirred for 1 h at 100° C. under nitrogen atmosphere. After completion of the reaction, the reaction mixture was cooled to rt and purified directly by reverse phase flash chromatography (Column: WelFlash™ C18-I, 20-40 m, 330 g; Eluent A: Water (plus 10 mmol/L NH4HCO₃); Eluent B: ACN; Gradient: 50%-95% B in 30 min; Flow rate: 80 mL/min; Detector: 220/254 nm; desired fractions were collected at 65% B) and concentrated under reduced pressure to afford the title compound (500 mg, 70% yield) as a yellow solid. LC/MS (ESI, m/z): [(M+1)]⁺=592.4.

Step 2—N-cyclopropyl-6-(4-(3-fluoro-5-formylpyridin-2-yl)indolin-1-yl)-8-(methylamino)imidazo[1,2-b]pyridazine-3-carboxamide trifluoroacetate. To a stirred solution of N-cyclopropyl-6-(4-(3-fluoro-5-formylpyridin-2-yl)indolin-1-yl)-8-((4-methoxybenzyl)(methyl)amino)imidazo[1,2-b]pyridazine-3-carboxamide (500 mg, 0.845 mmol) in DCM (6 mL) was added TFA (2 mL) at rt and the mixture was stirred for 1 h at it. On completion, the mixture was concentrated under vacuum. The residue was purified by trituration with ethyl ether (50 mL) to afford the title compound (500 mg, TFA salt) as an orange solid. LC/MS (ESI, m/z): [(M+1)]⁺=472.4.

### N-cyclopropyl-6-(4-(5-((5,5-difluoro-2,7-diazaspiro[3.5]nonan-2-yl)methyl)-3-fluoropyridin-2-yl)indolin-1-yl)-8-(methylamino)imidazo[1,2-b]pyridazine-3-carboxamide trifluoroacetate (Intermediate OH)

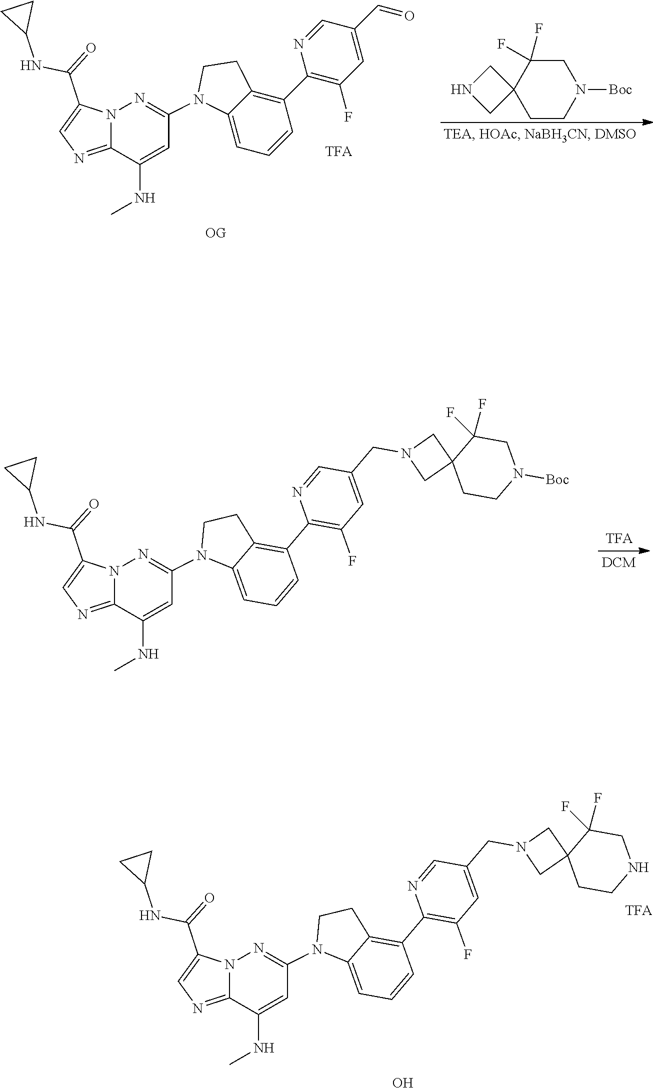

Step 1—Tert-butyl 2-((6-(1-(3-(cyclopropylcarbamoyl)-8-(methylamino)imidazo[1,2-b]pyridazin-6-yl)indolin-4-yl)-5-fluoropyridin-3-yl)methyl)-5,5-difluoro-2,7-diazaspiro[3.5]nonane-7-carboxylate. To a stirred solution of N-cyclopropyl-6-(4-(3-fluoro-5-formylpyridin-2-yl)indolin-1-yl)-8-(methylamino)imidazo[1,2-b]pyridazine-3-carboxamide 2,2,2-trifluoroacetate (200 mg, 0.424 mmol, Intermediate OG) in DMSO (5 mL) were added TEA (43 mg, 0.42 mmol), tert-butyl 5,5-difluoro-2,7-diazaspiro[3.5]nonane-7-carboxylate (111 mg, 0.424 mmol) and HOAc (76 mg, 1.3 mmol) at rt. The resulting mixture was stirred for 2 h at 50° C. The mixture was allowed to cool down to rt. Then NaBH₃CN (107 mg, 1.696 mmol) was added in portions to the mixture at rt. The resulting mixture was stirred overnight at 50° C. After completion of the reaction, the reaction mixture was cooled to rt and purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 50%- 95% B in 30 min; Flow rate: 80 mL/min; Detector: 220/254 nm; desired fractions were collected at 67% B) and concentrated under reduced pressure to afford to give the title compound (150 mg, 49% yield) as a white solid. LC/MS (ESI, m/z): [(M+1)]⁺=718.4.

Step 2—N-cyclopropyl-6-(4-(5-((5,5-difluoro-2,7-diazaspiro[3.5]nonan-2-yl)methyl)-3-fluoropyridin-2-yl)indolin-1-yl)-8-(methylamino)imidazo[1,2-b]pyridazine-3-carboxamide trifluoroacetate. To a stirred solution of tert-butyl 2-((6-(1-(3-(cyclopropylcarbamoyl)-8-(methylamino)imidazo[1,2-b]pyridazin-6-yl)indolin-4-yl)-5-fluoropyridin-3-yl)methyl)-5,5-difluoro-2,7-diazaspiro[3.5]nonane-7-carboxylate (150 mg, 0.209 mmol) in DCM (3 mL) was added TFA (1 mL) at rt and the mixture was stirred for 1 h at rt. On completion, the mixture was concentrated under vacuum. The residue was purified by trituration with ethyl ether (10 mL) to give the title compound (150 mg, TFA salt) as a light yellow solid. LC/MS (ESI, m/z): [(M+1)]⁺=618.4.

### Tert-butyl (R)-5,5-difluoro-2-(1-(6-(indolin-4-yl)pyridin-3-yl)ethyl)-2,7-diazaspiro[3.5]nonane-7-carboxylate (Intermediate OI)

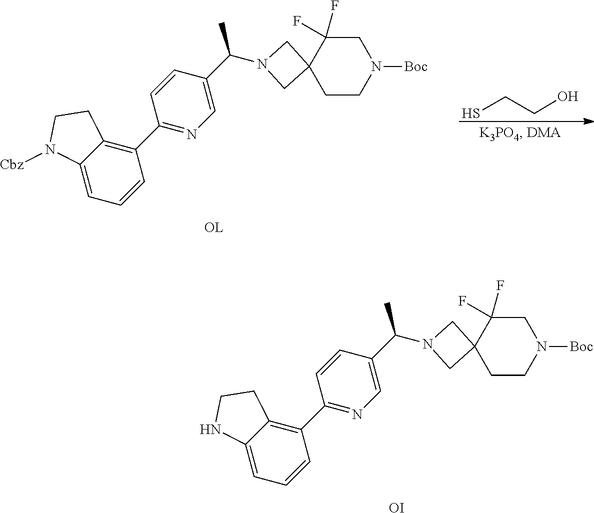

To a stirred solution of tert-butyl 2-(1-(6-(1-((benzyloxy)carbonyl)indolin-4-yl)pyridin-3-yl)ethyl)-5,5-difluoro-2,7-diazaspiro[3.5]nonane-7-carboxylate (490 mg, 0.79 mmol, Intermediate OL) and 0-mercaptoethanol (123 mg, 1.58 mmol) in DMA (10 mL) was added K3PO4 (672 mg, 3.16 mmol) at rt under nitrogen atmosphere. The resulting mixture was stirred for 1 h at 100° C. under nitrogen atmosphere. On completion, the mixture was cooled to rt and concentrated under vacuum. The residue was purified by reverse phase flash chromatography (Column: WelFlash™ C18-I, 20-40 m, 120 g; Eluent A: Water (10 mmol/L NH4HCO3); Eluent B: ACN; Gradient: 50%-80% B in 40 min; Flow rate: 60 mL/min; Detector: 220/254 nm; desired fractions were collected at 60% B) and concentrated under reduced pressure to afford the title compound (280 mg, 72% yield) as a yellow solid. LC/MS (ESI, m/z): [(M+1)]⁺=485.3.

### 6-(4-(5-((R)-1-(5,5-difluoro-2,7-diazaspiro[3.5]nonan-2-yl)ethyl)pyridin-2-yl)indolin-1-yl)-N-((1R,2R)-2-methoxycyclobutyl)-8-(methylamino)imidazo[1,2-b]pyridazine-3-carboxamide trifluoroacetate (Intermediate OJ)

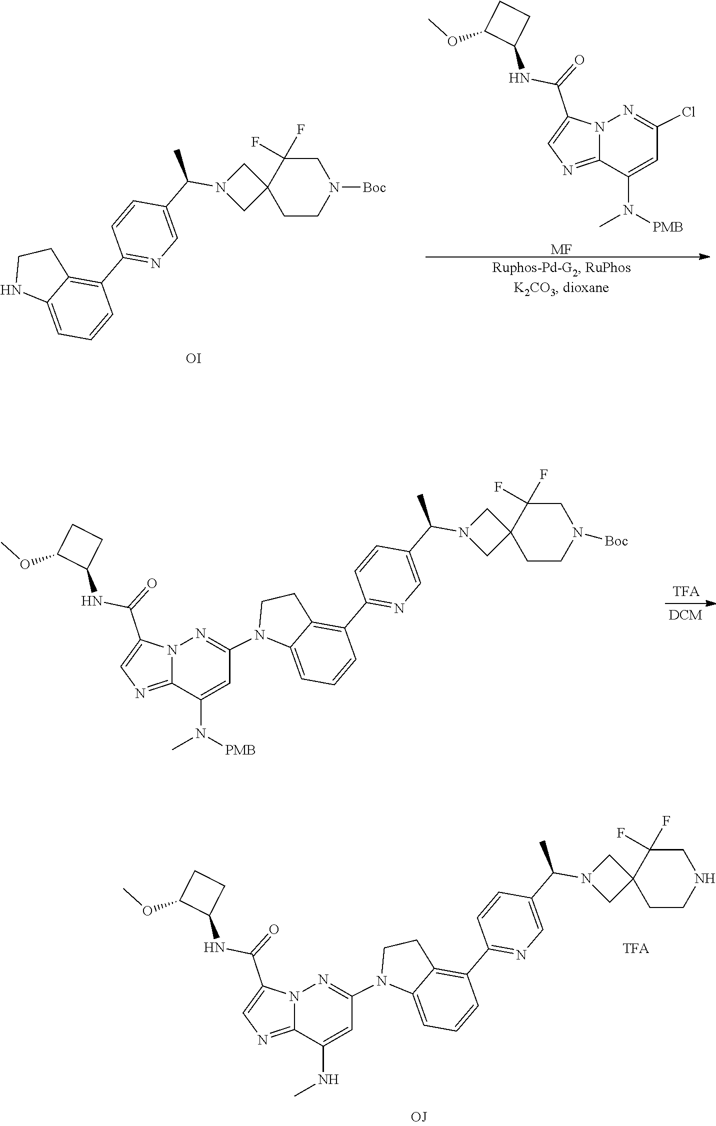

Step 1—Tert-butyl 5,5-difluoro-2-((R)-1-(6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate. To a stirred solution of tert-butyl (R)-5,5-difluoro-2-(1-(6-(indolin-4-yl)pyridin-3-yl)ethyl)-2,7-diazaspiro[3.5]nonane-7-carboxylate (280 mg, 0.57 mmol, Intermediate O1) and 6-chloro-N-[(1R,2R)-2-methoxycyclobutyl]-8-{[(4-methoxyphenyl)methyl](methyl)amino}imidazo[1,2-b]pyridazine-3-carboxamide (248.40 mg, 0.57 mmol, Intermediate MF) in dioxane (6 mL) were added RuPhos (53.93 mg, 0.11 mmol), RuPhos-PdOMs-2nd G (96.77 mg, 0.11 mmol) and K₂CO₃ (239.57 mg, 1.73 mmol) in portions at rt under nitrogen atmosphere. The resulting mixture was stirred for 2 h at 100° C. under nitrogen atmosphere. On completion, the mixture was cooled to rt and concentrated under reduced pressure. The residue was purified by silica gel column chromatography, eluted with EA, to afford the title compound (420 mg, 82% yield) as a yellow solid. LC/MS (ESI, m/z): [(M+1)]⁺=878.4.

Step 2—6-(4-(5-((R)-1-(5,5-difluoro-2,7-diazaspiro[3.5]nonan-2-yl)ethyl)pyridin-2-yl)indolin-1-yl)-N-((1R,2R)-2-methoxycyclobutyl)-8-(methylamino)imidazo[1,2-b]pyridazine-3-carboxamide trifluoroacetate. To a stirred solution of tert-butyl 5,5-difluoro-2-((R)-1-(6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate (200 mg, 0.22 mmol) in DCM (6 mL) was added TFA (2 mL) dropwise at rt under nitrogen atmosphere and the mixture was stirred for 1 h at rt under nitrogen atmosphere. On completion, the mixture was concentrated under reduced pressure. The residue was purified by trituration with Et₂O (20 mL) to afford the title compound (140 mg, 93% yield) as a yellow solid. LC/MS (ESI, m/z): [(M+1)]⁺=658.3.

### Benzyl 4-(4,4,5,5-tetramethyl-1,3,2-dioxaborolan-2-yl)-2,3-dihydroindole-1-carboxylate (Intermediate OK)

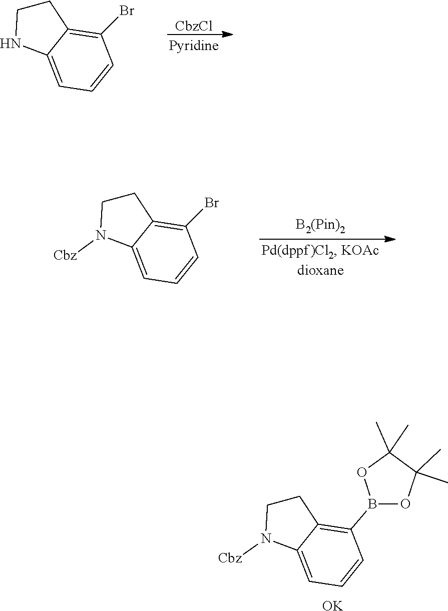

Step 1—Benzyl 4-bromo-2,3-dihydroindole-1-carboxylate. To a stirred solution of 4-bromo-2,3-dihydro-TH-indole (20 g, 100 mmol) and pyridine (39.94 g, 504.9 mmol) in DCM (400 mL) was added CbzCl (86.13 g, 504.9 mmol) at rt under nitrogen atmosphere. The resulting mixture was stirred for 1 h at rt under nitrogen atmosphere. On completion, the mixture was concentrated under reduced pressure. The resulting mixture was diluted with water (600 mL) and extracted with CH₂Cl₂ (3×200 mL). The combined organic layers were washed with brine (3×300 mL), dried over anhydrous Na₂SO₄, filtered, and the filtrate was concentrated under reduced pressure. The residue was purified by silica gel column chromatography, eluted with PE/EA (10:1), to afford the title compound (20 g, 60% yield) as a white solid. 1H NMR (400 MHz, Chloroform-d) δ 7.88-7.84 (m, 1H), 7.48-7.32 (m, 5H), 7.16-7.10 (m, 1H), 7.10-7.06 (m, 1H), 5.29 (s, 2H), 4.10 (t, J=8.8 Hz, 2H), 3.13 (t, J=8.7 Hz, 2H).

Step 2—Benzyl 4-(4,4,5,5-tetramethyl-1,3,2-dioxaborolan-2-yl)-2,3-dihydroindole-1-carboxylate. To a stirred solution of benzyl 4-bromo-2,3-dihydroindole-1-carboxylate (2 g, 6 mmol) and 4,4,5,5-tetramethyl-2-(4,4,5,5-tetramethyl-1,3,2-dioxaborolan-2-yl)-1,3,2-dioxaborolane (1.53 g, 6.02 mmol) in dioxane (30 mL) were added KOAc (1.77 g, 18.1 mmol) and Pd(dppf)C₁₋₂CH₂Cl₂ (0.49 g, 0.602 mmol) in portions at rt under nitrogen atmosphere. The resulting mixture was stirred for 3 h at 90° C. under nitrogen atmosphere. On completion, the mixture was cooled to rt and concentrated under vacuum. The residue was purified by silica gel column chromatography, eluted with PE/EA (5:1), to afford the title compound (1.9 g, 83% yield) as a yellow solid. ¹H NMR (300 MHz, Chloroform-d) δ 7.4-7.34 (m, 6H), 7.22-7.13 (m, 1H), 5.28 (s, 2H), 4.03 (t, J=8.7 Hz, 2H), 3.31 (t, J=8.7 Hz, 2H), 1.33 (s, 12H).

### Tert-butyl (R)-2-(1-(6-(1-((benzyloxy)carbonyl)indolin-4-yl)pyridin-3-yl)ethyl)-5,5-difluoro-2,7-diazaspiro[3.5]nonane-7-carboxylate (Intermediate OL) and tert-butyl (S)-2-(1-(6-(1-((benzyloxy)carbonyl)indolin-4-yl)pyridin-3-yl)ethyl)-5,5-difluoro-2,7-diazaspiro[3.5]nonane-7-carboxylate (Intermediate OM)

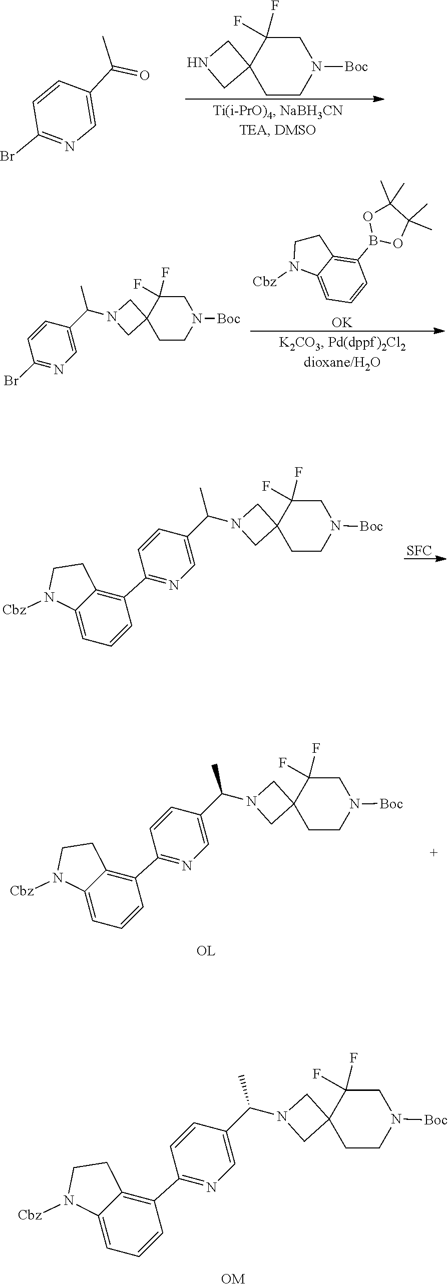

Step 1—Tert-butyl 2-[1-(6-bromopyridin-3-yl)ethyl]-5,5-difluoro-2,7-diazaspiro[3.5]nonane-7-carboxylate. To a stirred solution of tert-butyl 5,5-difluoro-2,7-diazaspiro[3.5]nonane-7-carboxylate (1 g, 4 mmol, CAS #139042-59-4) and TEA (0.39 g, 3.81 mmol) in DMSO (15 mL) were added 1-(6-bromopyridin-3-yl)ethanone (0.76 g, 3.81 mmol) and Ti(Oi-Pr)₄ (4.33 g, 15.25 mmol) in portions at rt under nitrogen atmosphere. The resulting mixture was stirred for 2 h at 80° C. under nitrogen atmosphere. To the above mixture was added NaBH₃CN (0.96 g, 15.25 mmol) in portions over 2 min at rt. The resulting mixture was stirred for an additional 16 h at 50° C. On completion, the mixture was cooled to rt and concentrated under vacuum. The residue was purified by reverse phase flash chromatography (Column: WelFlash™ C18-J, 20-40 μm, 330 g; Eluent A: Water (10 mmol/L NH₄HCO₃); Eluent B: ACN; Gradient: 50%-80% B in 40 min; Flow rate: 80 mL/min; Detector: 220/254 nm; desired fractions were collected at 68% B) and concentrated under reduced pressure to afford the title compound (1.4 g, 82% yield) as a yellow solid. LC/MS (ESI, m/z): [(M+H)]⁺=446.2.

Step 2—Tert-butyl 2-[1-(6-{1-[(benzyloxy)carbonyl]-2,3-dihydroindol-4-yl}pyridin-3-yl)ethyl]-5,5-difluoro-2,7-diazaspiro[3.5]nonane-7-carboxylate. To a stirred solution of tert-butyl 2-[1-(6-bromopyridin-3-yl)ethyl]-5,5-difluoro-2,7-diazaspiro[3.5]nonane-7-carboxylate (1.40 g, 3.14 mmol) and benzyl 4-(4,4,5,5-tetramethyl-1,3,2-dioxaborolan-2-yl)-2,3-dihydroindole-1-carboxylate (1.31 g, 3.45 mmol, Intermediate OK) in dioxane (10 mL) and H₂O (2 mL) were added K₂CO₃ (1.30 g, 9.41 mmol) and Pd(dppf)Cl₂CH₂Cl₂ (0.26 g, 0.31 mmol) in portions at it under nitrogen atmosphere. The resulting mixture was stirred for 2 h at 90° C. under nitrogen atmosphere. On completion, the mixture was cooled to rt and concentrated under reduced pressure. The residue was purified by silica gel column chromatography, eluted with PE/EA (5:1), to afford the title compound (1.5 g, 77% yield) as a yellow oil. LC/MS (ESI, m/z): [(M+H)]⁺=619.3.

Step 3—Tert-butyl (R)-2-(1-(6-(1-((benzyloxy)carbonyl)indolin-4-yl)pyridin-3-yl)ethyl)-5,5-difluoro-2,7-diazaspiro[3.5]nonane-7-carboxylate and tert-butyl (S)-2-(1-(6-(1-((benzyloxy)carbonyl)indolin-4-yl)pyridin-3-yl)ethyl)-5,5-difluoro-2,7-diazaspiro[3.5]nonane-7-carboxylate. Racemic tert-butyl 2-[1-(6-{1-[(benzyloxy)carbonyl]-2,3-dihydroindol-4-yl}pyridin-3-yl)ethyl]-5,5-difluoro-2,7-diazaspiro[3.5]nonane-7-carboxylate (1.5 g, 2.43 mmol) was separated by Prep-chiral-SFC (Column: CHIRALART Cellulose-SB, 3*25 cm, 5 m; Mobile Phase A: Hex(0.5% 2M NH3-MeOH)—SFC, Mobile Phase B: MeOH: DCM=1: 1—SFC; Flow rate: 100 mL/min; Gradient: 10% to 50% in 2.0 min, hold 1.0 min at 50%; Wave Length: 254 nm; RT1(min): 1.68; RT2(min): 2.10; Sample Solvent: MeOH: DCM=1: 1—SFC; Injection Volume: 0.6 mL; Number Of Runs: 80). The fractions of faster eluting peak was collected and concentrated under vacuum to afford tert-butyl (R)-2-(1-(6-(1-((benzyloxy)carbonyl)indolin-4-yl)pyridin-3-yl)ethyl)-5,5-difluoro-2,7-diazaspiro[3.5]nonane-7-carboxylate (490 mg, 33% yield) as an off-white solid. LC/MS (ESI, m/z): [(M+1)]⁺=619.3. The fractions of slower eluting peak was collected and concentrated under vacuum to afford tert-butyl (S)-2-(1-(6-(1-((benzyloxy)carbonyl)indolin-4-yl)pyridin-3-yl)ethyl)-5,5-difluoro-2,7-diazaspiro[3.5]nonane-7-carboxylate (520 mg, 35% yield) as an off-white solid. LC/MS (ESI, m/z): [(M+1)]J=619.3. The absolute stereochemistry of the enantiomers was assigned arbitrarily.

### Tert-butyl (S)-5,5-difluoro-2-(1-(6-(indolin-4-yl)pyridin-3-yl)ethyl)-2,7-diazaspiro[3.5]nonane-7-carboxylate (Intermediate ON)

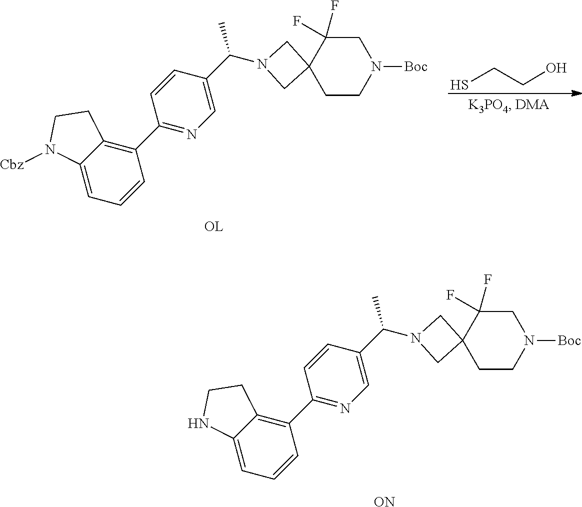

To a stirred solution of tert-butyl (S)-2-(1-(6-(1-((benzyloxy)carbonyl)indolin-4-yl)pyridin-3-yl)ethyl)-5,5-difluoro-2,7-diazaspiro[3.5]nonane-7-carboxylate (520 mg, 0.84 mmol, Intermediate OM) in DMA (10 mL) was added K3PO4 (713.58 mg, 3.36 mmol) and 2-mercaptoethan-1-ol (131.33 mg, 1.68 mmol) at rt under air atmosphere. The resulting mixture was stirred for 16 h at 100° C. On completion, the mixture was cooled and concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 45%-70% B in 25 min; Flow rate: 80 mL/min; Detector: 220/254 nm; desired fractions were collected at 62% B) and concentrated under reduced pressure to afford the title compound (300 mg, 74% yield) as a white solid. LC/MS (ESI, m/z): [(M+H)]⁺=485.3.

### 6-(4-(5-((S)-1-(5,5-difluoro-2,7-diazaspiro[3.5]nonan-2-yl)ethyl)pyridin-2-yl)indolin-1-yl)-N-((1R,2R)-2-methoxycyclobutyl)-8-(methylamino)imidazo[1,2-b]pyridazine-3-carboxamide trifluoroacetate (Intermediate 00)

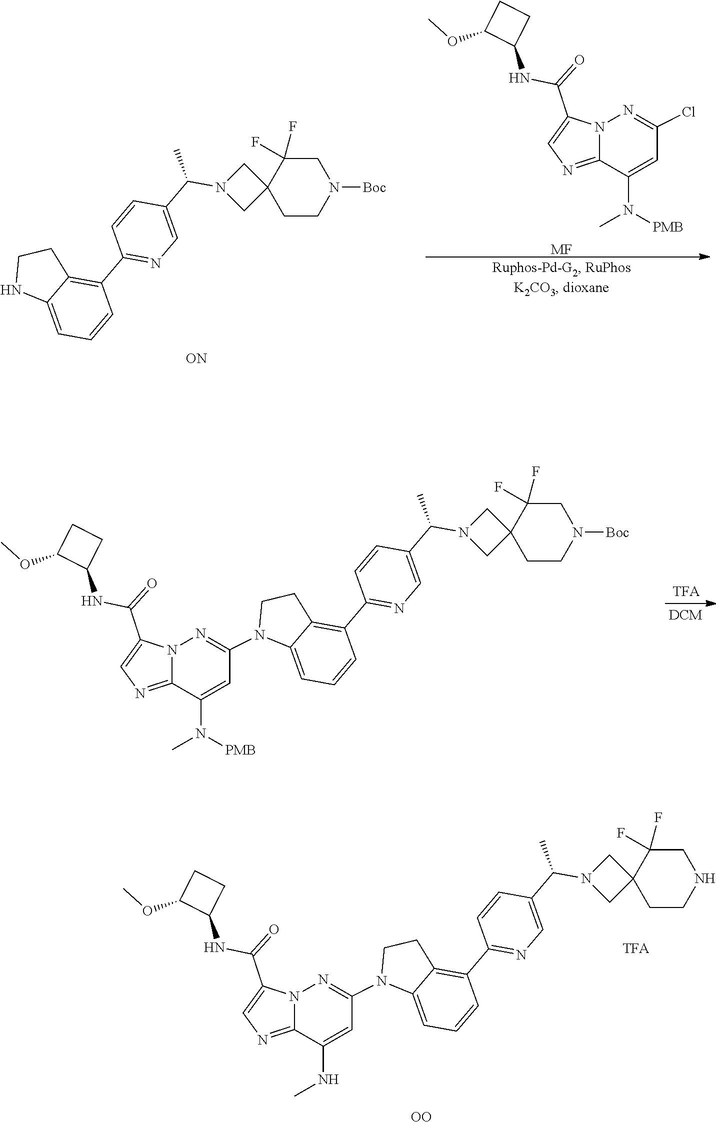

Step 1—Tert-butyl 5,5-difluoro-2-((S)-1-(6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate. To a stirred solution of tert-butyl (S)-5,5-difluoro-2-(1-(6-(indolin-4-yl)pyridin-3-yl)ethyl)-2,7-diazaspiro[3.5]nonane-7-carboxylate (300 mg, 0.62 mmol, Intermediate ON) and 6-chloro-8-((4-methoxybenzyl)(methyl)amino)-N-((1R,2R)-2-methoxycyclobutyl)imidazo[1,2-b]pyridazine-3-carboxamide (266.15 mg, 0.62 mmol, Intermediate MF) in dioxane (10 mL) were added RuPhos (57.78 mg, 0.12 mmol), RuPhos-PdCl-2nd G (96.30 mg, 0.12 mmol) and K₂CO₃ (256.68 mg, 1.86 mmol) at rt under nitrogen atmosphere. The resulting mixture was stirred for additional 2 h at 100° C. On completion, the mixture was cooled to rt and concentrated under reduced pressure. The residue was purified by silica gel column chromatography, eluted with EA, to afford the title compound (400 mg, 74% yield) as a yellow solid. LC/MS (ESI, m/z): [(M+H)]⁺=878.4.

Step 2—6-(4-(5-((S)-1-(5,5-difluoro-2,7-diazaspiro[3.5]nonan-2-yl)ethyl)pyridin-2-yl)indolin-1-yl)-N-((1R,2R)-2-methoxycyclobutyl)-8-(methylamino)imidazo[1,2-b]pyridazine-3-carboxamide trifluoroacetate. To a stirred solution of tert-butyl 5,5-difluoro-2-((S)-1-(6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate (200 mg, 0.23 mmol) in DCM (9 mL) was added TFA (3 mL) dropwise at rt and the mixture was stirred for 1 h at rt. On completion, the mixture was concentrated under reduced pressure. The residue was purified by trituration with Et₂O (30 mL) to afford the title compound (230 mg, 94% yield) as a yellow solid. LC/MS (ESI, m/z): [(M+H)]⁺=658.4.

### 5-Chloro-4-(2,4-dioxotetrahydropyrimidin-1(2H)-yl)picolinic acid (Intermediate OP)

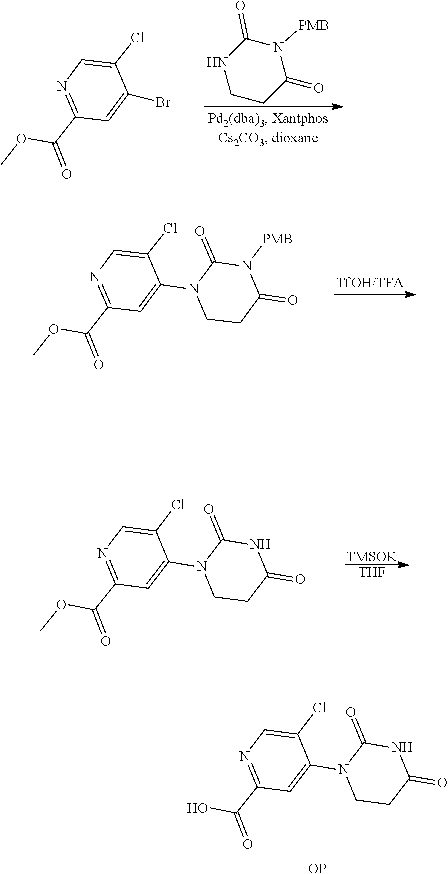

Step 1—Methyl 5-chloro-4-(3-(4-methoxybenzyl)-2,4-dioxotetrahydropyrimidin-1(2H)-yl)picolinate. To a stirred mixture of methyl 4-bromo-5-chloropicolinate (5.00 g, 20.0 mmol), 3-(4-methoxybenzyl)dihydropyrimidine-2,4(1H,3H)-dione (4.91 g, 21.0 mmol) and Cs₂CO₃ (19.51 g, 59.88 mmol) in dioxane (50 mL) were added Pd₂(dba)₃ (3.66 g, 3.99 mmol) and XantPhos (2.31 g, 3.99 mmol) at rt under nitrogen atmosphere. The resulting mixture was stirred for 2 h at 100° C. under nitrogen atmosphere. On completion, the mixture was cooled to rt and concentrated under reduced pressure. The residue was purified by silica gel column chromatography, eluted with CH₂Cl₂/EtOAc (2:1), to afford the title compound (5.10 g, 63% yield) as a light yellow solid. LC/MS (ESI, m/z): [(M+1)]=404.1.

Step 2—Methyl 5-chloro-4-(2,4-dioxotetrahydropyrimidin-1(2H)-yl)picolinate. To a stirred solution of methyl 5-chloro-4-(3-(4-methoxybenzyl)-2,4-dioxotetrahydropyrimidin-1(2H)-yl)picolinate (5.00 g, 12.4 mmol) in TFA (45 mL) was added trifluoromethanesulfonic acid (3 mL) dropwise at rt. The resulting mixture was stirred for 1 h at 70° C. On completion, the mixture was cooled to rt and concentrated under reduced pressure. The residue was purified by reversed-phase flash chromatography (Column: Spherical C18, 20-40 μm, 330 g; Mobile Phase A: Water (plus 10 mM NH₄CO₃), Mobile Phase B: ACN; Flow rate: 80 mL/min; Gradient (B %): 5%˜10%, 4 min; 10%˜20%, 20 min; 20%˜95%; 2 min; 95%, 5 min; Detector: 254 nm; the fractions containing desired product were collected at 15% B) and concentrated under reduced pressure to afford the title compound (2.20 g, 62% yield) as a white solid. LC/MS (ESI, m/z): [(M+1)]⁺=284.1.

Step 3—5-Chloro-4-(2,4-dioxotetrahydropyrimidin-1(2H)-yl)picolinic acid. To a stirred mixture of methyl 5-chloro-4-(2,4-dioxotetrahydropyrimidin-1(2H)-yl)picolinate (2.00 g, 7.05 mmol) in THF (40 mL) was added potassium trimethylsilanolate (9.04 g, 70.5 mmol) in portions at rt. The resulting mixture was stirred for 1 h at rt. On completion, the mixture was acidified to pH 3 with conc. HCl. The precipitated solids were collected by filtration and washed with water (3×10 mL) to afford the title compound (1.60 g, 84% yield) as a white solid. LC/MS (ESI, m/z): [(M+1)]⁺=270.1.

### 6-Chloro-N-((1R,2R)-2-ethoxycyclobutyl)-8-((4-methoxybenzyl)(methyl)amino)imidazo[1,2-b]pyridazine-3-carboxamide (Intermediate OQ)

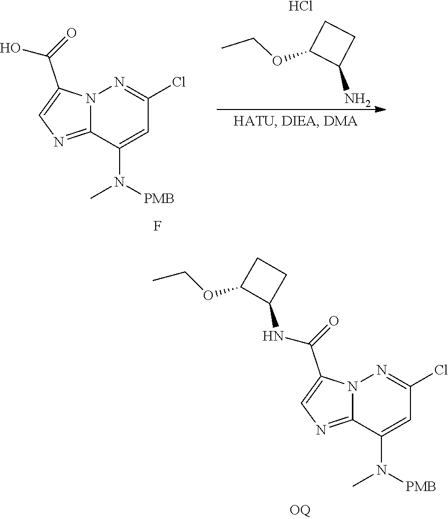

A solution of 6-chloro-8-((4-methoxybenzyl)(methyl)amino)imidazo[1,2-b]pyridazine-3-carboxylic acid (400 mg, 1.15 mmol, Intermediate F), DIEA (596 mg, 4.62 mmol), HATU (657 mg, 1.73 mmol) and (1R,2R)-2-ethoxycyclobutan-1-amine hydrochloride (175 mg, 1.15 mmol) in DMA (6 mL) was stirred for 1 h at rt. On completion, the mixture was purified by reversed-phase flash chromatography (Column: Spherical C18, 20˜40 μm, 330 g; Mobile Phase A: Water (plus 10 mM NH₄HCO₃), Mobile Phase B: ACN; Flow rate: 80 mL/min; Gradient (B %): 5%˜60%, 4 min; 60%˜90%, 20 min; 90%˜95%; 2 min; 95%, 5 min; Detector: 254 nm; the fractions containing desired product were collected at 80% B) and concentrated under reduced pressure to afford the title compound (475 mg, 92% yield) as a yellow solid. LC/MS (ESI, m/z): [(M+1)]⁺=444.2.

### N-((1R,2R)-2-ethoxycyclobutyl)-6-(4-(3-fluoro-5-formylpyridin-2-yl)indolin-1-yl)-8-(methylamino)imidazo[1,2-b]pyridazine-3-carboxamide trifluoroacetate (Intermediate OR)

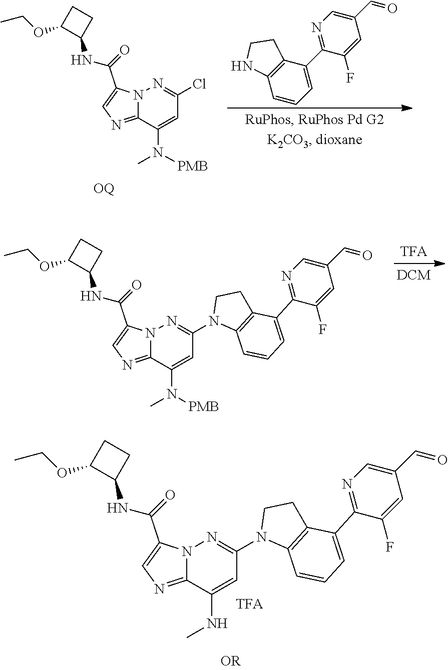

Step 1—N-((1R,2R)-2-ethoxycyclobutyl)-6-(4-(3-fluoro-5-formylpyridin-2-yl)indolin-1-yl)-8-((4-methoxybenzyl)(methyl)amino)imidazo[1,2-b]pyridazine-3-carboxamide. To a stirred solution of 6-chloro-N-((1R,2R)-2-ethoxycyclobutyl)-8-((4-methoxybenzyl)(methyl)amino)imidazo[1,2-b]pyridazine-3-carboxamide (200 mg, 0.451 mmol, Intermediate OQ) and 5-fluoro-6-(indolin-4-yl)nicotinaldehyde (109 mg, 0.451 mmol, Intermediate MB) in dioxane (4 mL) were added RuPhos (21 mg, 0.045 mmol), RuPhos-PdCl-2nd G (35 mg, 0.045 mmol) and K₂CO₃ (187 mg, 1.35 mmol) at rt under nitrogen atmosphere. The resulting mixture was stirred for 2 h at 100° C. under nitrogen atmosphere. On completion, the mixture was concentrated under reduced pressure. The residue was purified by silica gel column chromatography, eluted with CH₂Cl₂/EtOAc (1:1), to afford the title compound (240 mg, 81% yield) as a yellow solid. LC/MS (ESI, m/z): [(M+1)]⁺=650.4.

Step 2—N-((1R,2R)-2-ethoxycyclobutyl)-6-(4-(3-fluoro-5-formylpyridin-2-yl)indolin-1-yl)-8-(methylamino)imidazo[1,2-b]pyridazine-3-carboxamide trifluoroacetate. A solution of N-((1R,2R)-2-ethoxycyclobutyl)-6-(4-(3-fluoro-5-formylpyridin-2-yl)indolin-1-yl)-8-((4-methoxybenzyl)(methyl)amino)imidazo[1,2-b]pyridazine-3-carboxamide (240 mg, 0.369 mmol) in DCM (6 mL) and TFA (2 mL) was stirred for 1 h at rt. On completion, the mixture was concentrated under reduced pressure. The resulting mixture was diluted with diethyl ether (20 mL). The precipitated solids were collected by filtration and washed with diethyl ether (2×10 mL) to give the title compound (208 mg, 87% yield) as a yellow solid. LC/MS (ESI, m/z): [(M+1)]⁺=530.3.

### 6-(4-(5-((5,5-Difluoro-2,7-diazaspiro[3.5]nonan-2-yl)methyl)-3-fluoropyridin-2-yl)indolin-1-yl)-N-((1R,2R)-2-ethoxycyclobutyl)-8-(methylamino)imidazo[1,2-b]pyridazine-3-carboxamide trifluoroacetate (Intermediate OS)

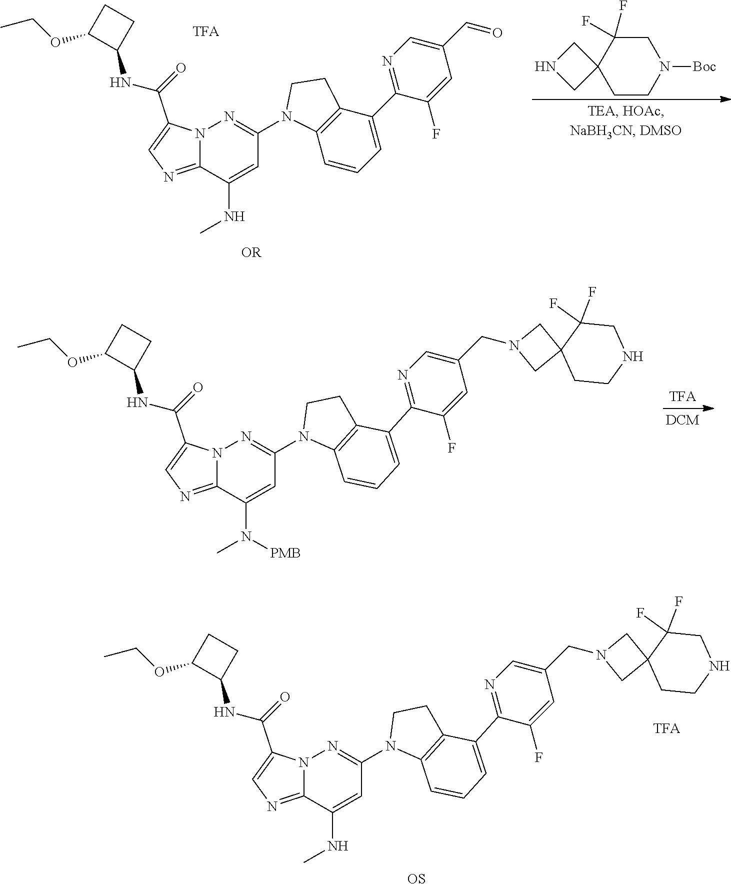

Step 1—Tert-butyl 2-((6-(1-(3-(((1R,2R)-2-ethoxycyclobutyl)carbamoyl)-8-(methylamino)imidazo[1,2-b]pyridazin-6-yl)indolin-4-yl)-5-fluoropyridin-3-yl)methyl)-5,5-difluoro-2,7-diazaspiro[3.5]nonane-7-carboxylate. To a stirred solution of N-((1R,2R)-2-ethoxycyclobutyl)-6-(4-(3-fluoro-5-formylpyridin-2-yl)indolin-1-yl)-8-(methylamino)imidazo[1,2-b]pyridazine-3-carboxamide trifluoroacetate (190 mg, 0,295 mmol, Intermediate OR) and TEA (30 mg, 0,295 mmol) in DMSO (4 mL) were added tert-butyl 5,5-difluoro-2,7-diazaspiro[3.5]nonane-7-carboxylate (77 mg, 0,295 mmol) and AcOH (88 mg, 1.475 mmol) at rt. The resulting mixture was stirred for 1 h at 50° C. To the above mixture was added NaBH₃CN (93 mg, 1.475 mmol) at rt. The resulting mixture was stirred for an additional 1 h at 50° C. On completion, the mixture was concentrated under reduced pressure. The residue was purified by reversed-phase flash chromatography (Column: Spherical C18, 20˜40 μm, 120 g; Mobile Phase A: Water (plus 10 mM NH₄HCO₃). Mobile Phase B: ACN; Flow rate: 50 mL/min; Gradient (B %): 5%-50%, 4 min; 50%-80%, 20 min; 80%-95%; 2 min; 95%, 5 min; Detector: 254 nm; the fractions containing desired product were collected at 76% B) and concentrated under reduced pressure to afford the title compound (160 mg, 69% yield) as an off-white solid. LC/MS (ESI, m/z): [(M+1)]⁺=776.4.

Step 2—6-(4-(5-((5,5-Difluoro-2,7-diazaspiro[3.5]nonan-2-yl)methyl)-3-fluoropyridin-2-yl)indolin-1-yl)-N-((1R,2R)-2-ethoxycyclobutyl)-8-(methylamino)imidazo[1,2-b]pyridazine-3-carboxamide trifluoroacetate. To a solution of tert-butyl 2-((6-(1-(3-(((1R,2R)-2-ethoxycyclobutyl)carbamoyl)-8-(methylamino)imidazo[1,2-b]pyridazin-6-yl)indolin-4-yl)-5-fluoropyridin-3-yl)methyl)-5,5-difluoro-2,7-diazaspiro[3.5]nonane-7-carboxylate (160 mg, 0.206 mmol) in DCM (5 mL) was TFA (1 mL) dropwise at rt and the mixture was stirred for 1 h at rt. On completion, the mixture was concentrated under reduced pressure. The residue was diluted with diethyl ether (20 mL). The precipitated solids were collected by filtration and washed with diethyl ether (2×10 mL) to give the title compound (260 mg, TFA salt) as a white solid. LC/MS (ESI, m/z): [(M+1)]⁺=676.3.

### 1-(2-Chloro-5-{5,5-difluoro-2,7-diazaspiro[3.5]nonane-2-carbonyl}phenyl)-1,3-diazinane-2,4-dione trifluoroacetate (Intermediate OT)

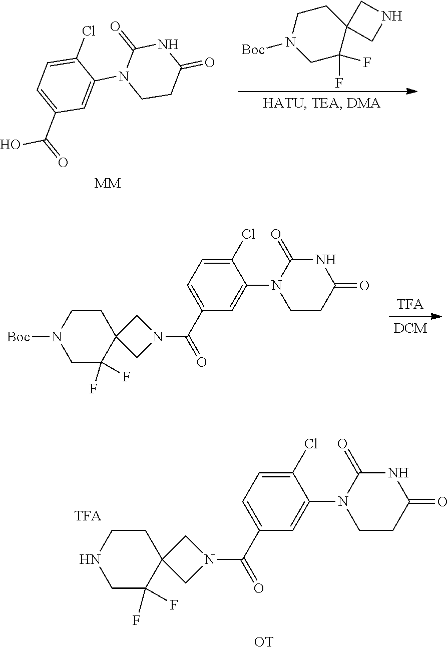

Step 1—Tert-butyl 2-[4-chloro-3-(2,4-dioxo-1,3-diazinan-1-yl)benzoyl]-5,5-difluoro-2,7-diazaspiro[3.5]nonane-7-carboxylate. To a stirred mixture of 4-chloro-3-(2,4-dioxo-1,3-diazinan-1-yl)benzoic acid (200 mg, 0.744 mmol, Intermediate MM) and tert-butyl 5,5-difluoro-2,7-diazaspiro[3.5]nonane-7-carboxylate (214.80 mg, 0.818 mmol) in DMF (4 mL) were added TEA (0.31 mL, 2.23 mmol) and HATU (424.61 mg, 1.116 mmol) at rt and the mixture was stirred for 1 h at rt. On completion, the mixture was concentrated under reduced pressure. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 35%-50% B in 25 min; Flow rate: 60 mL/min; Detector: 220/254 nm; desired fractions were collected at 42% B) and concentrated under reduced pressure to afford the title compound (290 mg, 76% yield) as a white solid. LC/MS (ESI, m/z): [(M−H)]⁻=511.2.

Step 2—1-(2-Chloro-5-{5,5-difluoro-2,7-diazaspiro[3.5]nonane-2-carbonyl}phenyl)-1,3-diazinane-2,4-dione trifluoroacetate. To a stirred solution of tert-butyl 2-[4-chloro-3-(2,4-dioxo-1,3-diazinan-1-yl)benzoyl]-5,5-difluoro-2,7-diazaspiro[3.5]nonane-7-carboxylate (290 mg, 0.565 mmol) in DCM (2 mL) was added TFA (1 mL) at rt and the mixture was stirred for 1 h at rt. On completion, the mixture was concentrated under reduced pressure. The residue was purified by trituration with Et₂O (20 mL) to afford the title compound (350 mg) as a white solid. LC/MS (ESI, m/z): [(M+H)]⁺=413.1.

### 6-(4-(((R)-3,3-difluoro-4-(3,9-diazaspiro[5.5]undecan-3-yl)piperidin-1-yl)methyl)-6-fluoroindolin-1-yl)-N-((1R,2R)-2-methoxycyclobutyl)-8-(methylamino)imidazo[1,2-b]pyridazine-3-carboxamide trifluoroacetate (Intermediate OU)

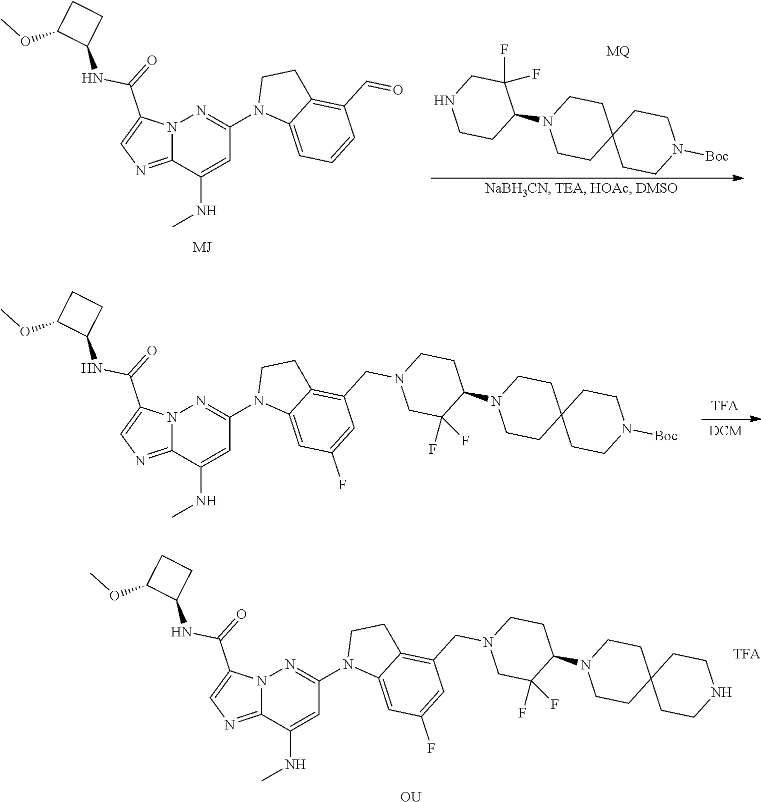

Step 1—Tert-butyl 9-((R)-3,3-difluoro-1-((6-fluoro-1-(3-(((1R,2R)-2-methoxycyclobutyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate. To a stirred solution of tert-butyl (S)-9-(3,3-difluoropiperidin-4-yl)-3,9-diazaspiro[5.5]undecane-3-carboxylate (420 mg, 1.13 mmol, Intermediate MQ) in DMSO (8 mL) were added TEA (180 mg, 1.78 mmol), 6-(6-fluoro-4-formylindolin-1-yl)-N-((1R,2R)-2-methoxycyclobutyl)-8-(methylamino)imidazo[1,2-b]pyridazine-3-carboxamide trifluoroacetate (500 mg, 1.14 mmol, Intermediate MJ) and HOAc (220 mg, 3.66 mmol) at rt. The resulting mixture was stirred for 1 h at 50° C. The mixture was allowed to cool down to rt. To the above mixture was added NaBH₃CN (220 mg, 3.501 mmol) at rt. Then the resulting mixture was stirred for additional 1 h at 50° C. After completion of the reaction, the reaction mixture was cooled to rt and purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 75%-95% B in 25 min; Flow rate: 80 mL/min; Detector: 220/254 nm; desired fractions were collected at 95% B) and concentrated under reduced pressure to give the title compound (340 mg, 37% yield) as a light yellow solid. LC/MS (ESI, m/z): [(M+1)]⁺=796.4.

Step 2—6-(4-(((R)-3,3-difluoro-4-(3,9-diazaspiro[5.5]undecan-3-yl)piperidin-1-yl)methyl)-6-fluoroindolin-1-yl)-N-((1R,2R)-2-methoxycyclobutyl)-8-(methylamino)imidazo[1,2-b]pyridazine-3-carboxamide trifluoroacetate. To a stirred solution of tert-butyl 9-((R)-3,3-difluoro-1-((6-fluoro-1-(3-(((1R,2R)-2-methoxycyclobutyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate (860 mg, 1.08 mmol) in DCM (10 mL) was added TFA (3.00 mL) at rt and the mixture was stirred for 1 h at rt. The resulting mixture was concentrated under reduced pressure. The residue was purified by trituration with Et₂O (20 mL) to give the title compound (750 mg, TFA salt) as an off-white solid. LC/MS (ESI, m/z): [(M+1)]=696.4.

### 6-(4-(((S)-3,3-difluoro-4-(3,9-diazaspiro[5.5]undecan-3-yl)piperidin-1-yl)methyl)-6-fluoroindolin-1-yl)-N-((1R,2R)-2-methoxycyclobutyl)-8-(methylamino)imidazo[1,2-b]pyridazine-3-carboxamide trifluoroacetate (Intermediate OV)

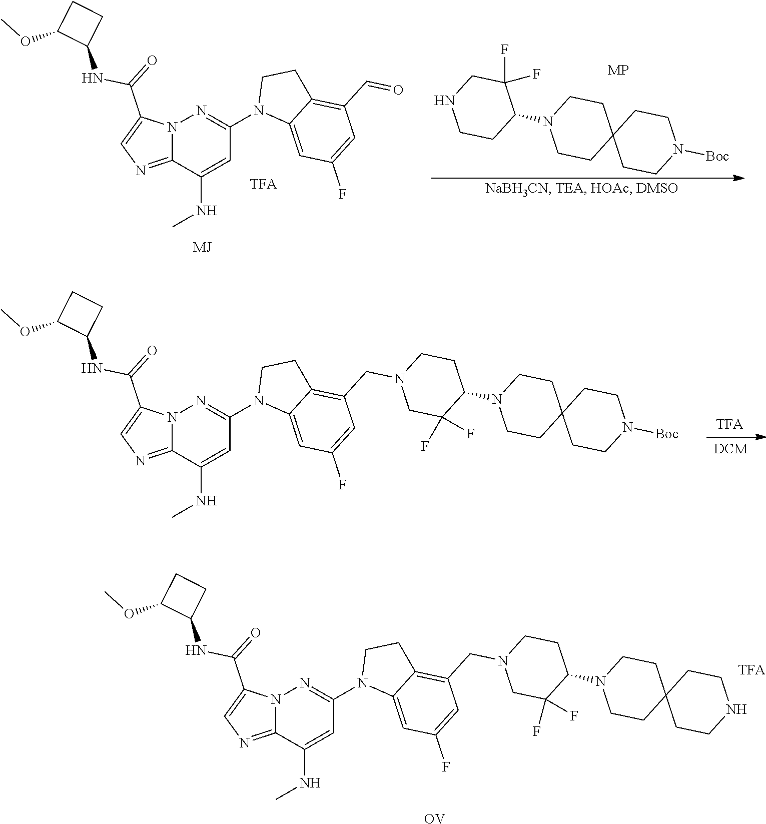

Step 1—Tert-butyl 9-((S)-3,3-difluoro-1-((6-fluoro-1-(3-(((1R,2R)-2-methoxycyclobutyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate. To a stirred solution of tert-butyl (R)-9-(3,3-difluoropiperidin-4-yl)-3,9-diazaspiro[5.5]undecane-3-carboxylate (820 mg, 2.196 mmol, Intermediate MP) in DMSO (10 mL) were added TEA (280 mg, 2.77 mmol), 6-(6-fluoro-4-formylindolin-1-yl)-N-((1R,2R)-2-methoxycyclobutyl)-8-(methylamino)imidazo[1,2-b]pyridazine-3-carboxamide trifluoroacetate (800 mg, 1.83 mmol) and HOAc (330 mg, 5.50 mmol, Intermediate MJ) at rt. The resulting mixture was stirred for 1 h at 50° C. The mixture was allowed to cool down to room temperature. To the above mixture was added NaBH₃CN (350 mg, 5.570 mmol) at rt. The resulting mixture was stirred for additional 16 h at 50° C. The After completion of the reaction, the reaction mixture was cooled to rt and purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 75%-95% B in 25 min; Flow rate: 80 mL/min; Detector: 220/254 nm; desired fractions were collected at 95% B) and concentrated under reduced pressure to give the title compound (540 mg, 37% yield) as a light yellow solid. LC/MS (ESI, m/z): [(M+1)]⁺=796.4.

Step 2—6-(4-(((S)-3,3-difluoro-4-(3,9-diazaspiro[5.5]undecan-3-yl)piperidin-1-yl)methyl)-6-fluoroindolin-1-yl)-N-((1R,2R)-2-methoxycyclobutyl)-8-(methylamino)imidazo[1,2-b]pyridazine-3-carboxamide trifluoroacetate. To a stirred solution of tert-butyl 9-((S)-3,3-difluoro-1-((6-fluoro-1-(3-(((1R,2R)-2-methoxycyclobutyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate (540 mg, 0.678 mmol) in DCM (6 mL) was added TFA (2 mL) at rt and the mixture was stirred for 1 h at rt. On completion, the resulting mixture was concentrated under reduced pressure. The residue was purified by trituration with Et₂O (10 mL) to give the title compound (470 mg, TFA salt) as a light yellow solid. LC/MS (ESI, m/z): [(M+1)]⁺=696.4.

### Tert-butyl 2-(1-(6-chloro-5-fluoropyridin-3-yl)ethyl)-5,5-difluoro-2,7-diazaspiro[3.5]nonane-7-carboxylate (Intermediate OW)

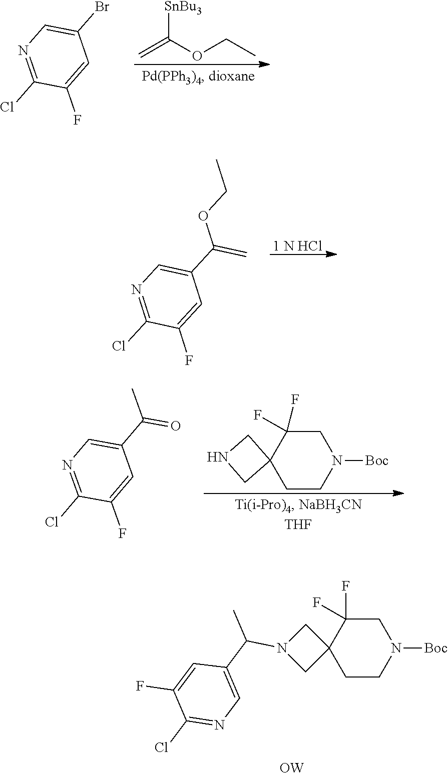

Step 1—2-Chloro-5-(1-ethoxyvinyl)-3-fluoropyridine. To a stirred mixture of 5-bromo-2-chloro-3-fluoropyridine (3 g, 15 mmol, CAS #831203-13-5) and tributyl(1-ethoxyvinyl) stannane (7.72 g, 21.4 mmol) in dioxane (20 mL) was added Pd(PPh₃)₄(1.65 g, 1.43 mmol) at rt under nitrogen atmosphere. The resulting mixture was stirred for 1 h at 100° C. under nitrogen atmosphere. On completion, the mixture was cooled to rt and concentrated under reduced pressure. The residue was purified by silica gel column chromatography, eluted with PE/EA (10:1), to afford the title compound (2.8 g, 97% yield) as a yellow solid. LC/MS (ESI, m/z): [(M+1)]⁺=202.2

Step 2—1-(6-Chloro-5-fluoropyridin-3-yl)ethan-1-one. 2-Chloro-5-(1-ethoxyvinyl)-3-fluoropyridine (3 g, 15 mmol) was dissolved in 1 N aq. HCl (20 mL) at rt. The resulting solution was stirred for 1 h at 50° C. On completion, the mixture was extracted with EtOAc (3×10 mL). The combined organic layers were washed with brine (3×20 mL), dried over anhydrous Na₂SO₄, filtered, and the filtrate was concentrated under reduced pressure to afford the title compound (2.2 g, 85% yield) as a yellow oil. LC/MS (ESI, m/z): [(M+1)]=174.3.

Step 3—Tert-butyl 2-(1-(6-chloro-5-fluoropyridin-3-yl)ethyl)-5,5-difluoro-2,7-diazaspiro[3.5]nonane-7-carboxylate. To a stirred solution of tert-butyl 5,5-difluoro-2,7-diazaspiro[3.5]nonane-7-carboxylate (1 g, 4 mmol) and TEA (0.19 g, 1.91 mmol) in THF (20 mL) were added 1-(6-chloro-5-fluoropyridin-3-yl)ethan-1-one (0.73 g, 4.19 mmol) and Ti(Oi-Pr)₄ (4.33 g, 15.3 mmol) at rt and the mixture was stirred for 2 h at rt. To the above mixture was added NaBH₃CN (0.96 g, 15.248 mmol) at rt. The resulting mixture was stirred for additional 16 h at 50° C. On completion, the mixture was cooled to rt and concentrated under vacuum. The residue was purified by reverse phase flash chromatography (WelFlash™ C18-I, 20-40 μm, 330 g; Eluent A: Water (plus 10 mmol/L NH₄HCO₃); Eluent B: ACN; Gradient: 25%-55% B in 25 min; Flow rate: 80 mL/min; Detector: 220 nm; desired fractions were collected at 50% B) and concentrated under reduced pressure to afford the title compound (700 mg, 41% yield) as a white solid. LC/MS (ESI, m/z): [(M+1)]⁺=420.3.

### Tert-butyl 5,5-difluoro-2-(1-(5-fluoro-6-(indolin-4-yl)pyridin-3-yl)ethyl)-2,7-diazaspiro[3.5]nonane-7-carboxylate (Intermediate OX)

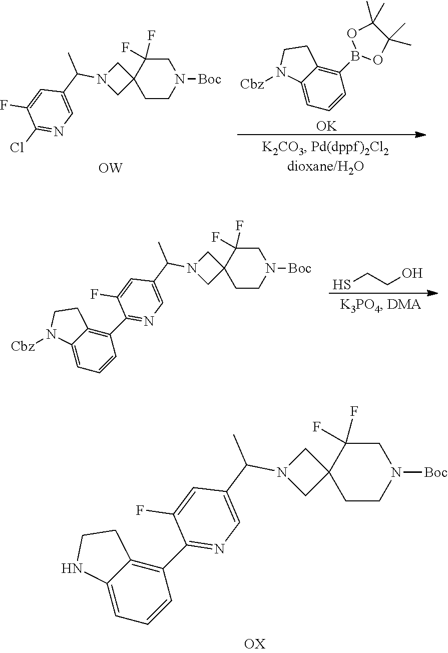

Step 1—Tert-butyl 2-(1-(6-(1-((benzyloxy)carbonyl)indolin-4-yl)-5-fluoropyridin-3-yl)ethyl)-5,5-difluoro-2,7-diazaspiro[3.5]nonane-7-carboxylate. To a stirred solution of tert-butyl 2-(1-(6-chloro-5-fluoropyridin-3-yl)ethyl)-5,5-difluoro-2,7-diazaspiro[3.5]nonane-7-carboxylate (700 mg, 1.67 mmol, Intermediate OW) and benzyl 4-(4,4,5,5-tetramethyl-1,3,2-dioxaborolan-2-yl)indoline-1-carboxylate (695.53 mg, 1.834 mmol, Intermediate OK) in dioxane (10 mL) and H₂O (2 mL) was added K₂CO₃ (691.24 mg, 5.001 mmol) and Pd(dppf)Cl₂CH₂Cl₂ (271.62 mg, 0.333 mmol) at rt under air atmosphere. The resulting mixture was stirred for 2 h at 90° C. under nitrogen atmosphere. On completion, the mixture was cooled to rt and concentrated under vacuum. The residue was purified by reverse phase flash chromatography (WelFlash™ C18-I, 20-40 μm, 330 g; Eluent A: Water (plus 10 mmol/L NH₄HCO₃); Eluent B: ACN; Gradient: 25%-55% B in 25 min; Flow rate: 80 mL/min; Detector: 254 nm; desired fractions were collected at 52% B) and concentrated under reduced pressure to afford the title compound (850 mg, 69% yield) as an off-white solid. LC/MS (ESI, m/z): [(M+1)]⁺=637.3.

Step 2—Tert-butyl 5,5-difluoro-2-(1-(5-fluoro-6-(indolin-4-yl)pyridin-3-yl)ethyl)-2,7-diazaspiro[3.5]nonane-7-carboxylate. To a stirred solution of tert-butyl 2-(1-(6-(1-((benzyloxy)carbonyl)indolin-4-yl)-5-fluoropyridin-3-yl)ethyl)-5,5-difluoro-2,7-diazaspiro[3.5]nonane-7-carboxylate (850 mg, 1.335 mmol) and K₃PO₄ (1133.47 mg, 5.340 mmol) in DMA (10 mL) was added 2-hydroxy-1-ethanethiol (208.61 mg, 2.670 mmol) at rt. The resulting mixture was stirred at 70° C. for 16 h. On completion, the mixture was cooled to rt and concentrated under vacuum. The residue was purified by reverse phase flash chromatography (WelFlash™ C18-I, 20-40 μm, 330 g; Eluent A: Water (plus 10 mmol/L NH₄HCO₃); Eluent B: ACN; Gradient: 25%-55% B in 25 min; Flow rate: 80 mL/min; Detector: 220 nm; desired fractions were collected at 50% B) and concentrated under reduced pressure to afford the title compound (300 mg, 33% yield) as a yellow solid. LC/MS (ESI, m/z): [(M+1)]⁺=503.3.

### Tert-butyl 5,5-difluoro-2-((S)-1-(5-fluoro-6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate (Intermediate OY) and tert-butyl 5,5-difluoro-2-((R)-1-(5-fluoro-6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate (Intermediate OZ)

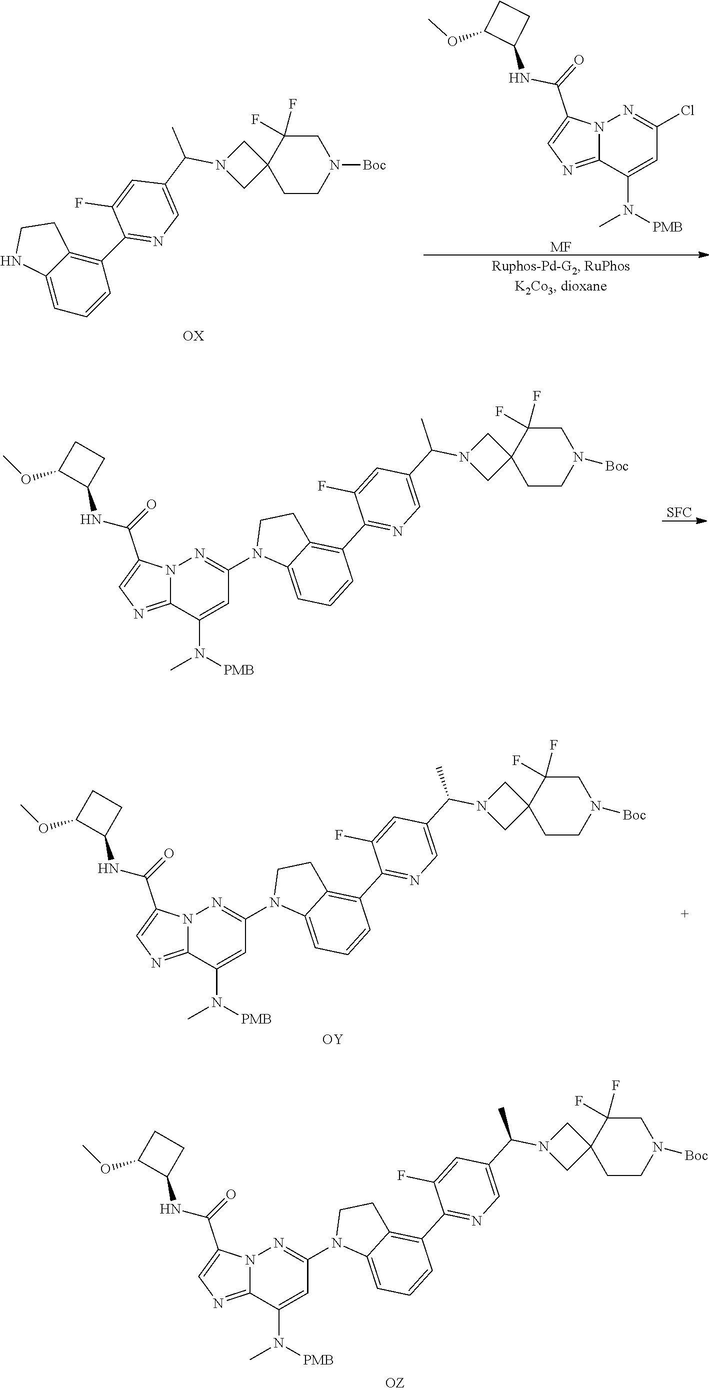

Step 1—Tert-butyl 5,5-difluoro-2-(1-(5-fluoro-6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate. To a stirred solution of tert-butyl 5,5-difluoro-2-(1-(5-fluoro-6-(indolin-4-yl)pyridin-3-yl)ethyl)-2,7-diazaspiro[3.5]nonane-7-carboxylate (300 mg, 0.597 mmol, Intermediate OX) and 6-chloro-8-((4-methoxybenzyl)(methyl)amino)-N-((1R,2R)-2-methoxycyclobutyl)imidazo[1,2-b]pyridazine-3-carboxamide (256.62 mg, 0.597 mmol, Intermediate MF) in dioxane (5 mL) were added K₂CO₃ (247.49 mg, 1.791 mmol). RuPhos (55.71 mg, 0.119 mmol) and RuPhos-PdCl-2nd G (46.42 mg, 0.060 mmol) in turns at rt under nitrogen atmosphere. The resulting mixture was stirred for an additional 2 h at 100° C. On completion, the mixture was cooled to rt and concentrated under vacuum. The residue was purified by reverse phase flash chromatography (WelFlash™ C18-I, 20-40 μm, 330 g; Eluent A: Water (plus 10 mmol/L NH₄HCO₃); Eluent B: ACN; Gradient: 25%-55% B in 25 min; Flow rate: 80 mL/min; Detector: 220 nm; desired fractions were collected at 85% B) and concentrated under reduced pressure to afford the title compound (350 mg, 65% yield) as an off-white solid. LC/MS (ESI, m/z): [(M+1)]⁺=896.4.

Step 2—Tert-butyl 5,5-difluoro-2-((S)-1-(5-fluoro-6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate and tert-butyl 5,5-difluoro-2-((R)-1-(5-fluoro-6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate. The mixture of diastereomers of tert-butyl 5,5-difluoro-2-(1-(5-fluoro-6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate (350 mg, 0.390 mmol) was separated by Prep-chiral-HPLC (Column: CHIRALPAK IC, 2*25 cm, 5 μm; Mobile Phase A: Hex(0.5% 2M NH3-MeOH)—HPLC, Mobile Phase B: EtOH: DCM=1: 1—HPLC; Flow rate: 20 mL/min; Gradient: 50% B to 50% B in 16 min; Wave Length: 220/254 nm; RT1(min): 11.2; RT2(min): 14.64; Sample Solvent: EtOH: DCM=1: 1-HPLC; Injection Volume: 0.5 mL; Number Of Runs: 11). The fractions of faster eluting peak was collected and concentrated under vacuum to afford tert-butyl 5,5-difluoro-2-((S)-1-(5-fluoro-6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate (120 mg, 34% yield) as an off-white solid. LC/MS (ESI, m/z): [(M+1)]⁺=896.4. The fractions of slower eluting peak was collected and concentrated under vacuum to afford tert-butyl 5,5-difluoro-2-((R)-1-(5-fluoro-6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate (1110 mg, 31% yield) as an off-white solid. LC/MS (ESI, m/z): [(M+1)]⁺=896.4. The absolute stereochemistry of the diastereomers was assigned arbitrarily.

### 6-(4-(5-((S)-1-(5,5-difluoro-2,7-diazaspiro[3.5]nonan-2-yl)ethyl)-3-fluoropyridin-2-yl)indolin-1-yl)-N-((1R,2R)-2-methoxycyclobutyl)-8-(methylamino)imidazo[1,2-b]pyridazine-3-carboxamide trifluoroacetate (Intermediate PA)

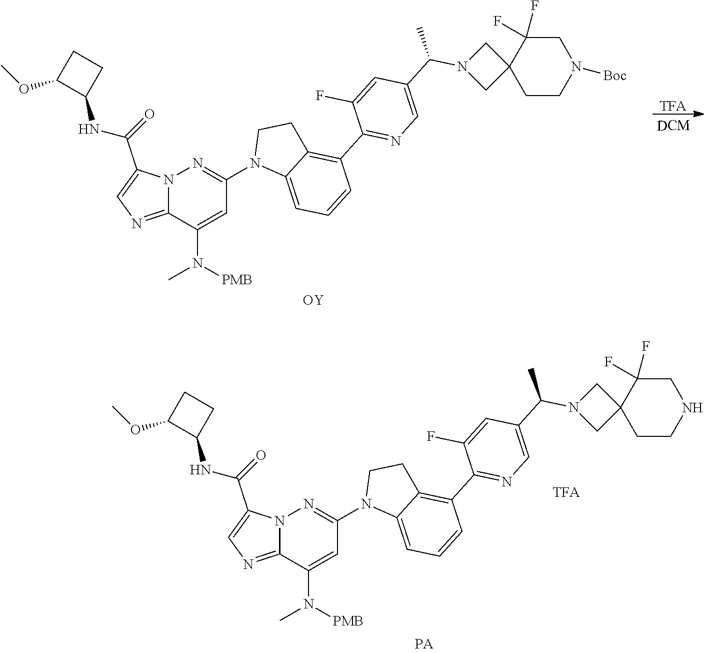

To a stirred solution of tert-butyl 5,5-difluoro-2-((S)-1-(5-fluoro-6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate (120 mg, 0.134 mmol, Intermediate OY) in DCM (3 mL) were added TFA (1 mL) at rt and the mixture was stirred for 1 h at rt under air atmosphere. On completion, the residue was purified by trituration with ethyl ether (30 mL) to give the title compound (90 mg, 99% yield) as a white solid. LC/MS (ESI, m/z): [(M+1)]=676.3.

### 6-(6-Fluoro-4-(3-fluoro-5-formylpyridin-2-yl)indolin-1-yl)-N-((1R,2S)-2-fluorocyclopropyl)-8-(methylamino)imidazo[1,2-b]pyridazine-3-carboxamide (Intermediate PB)

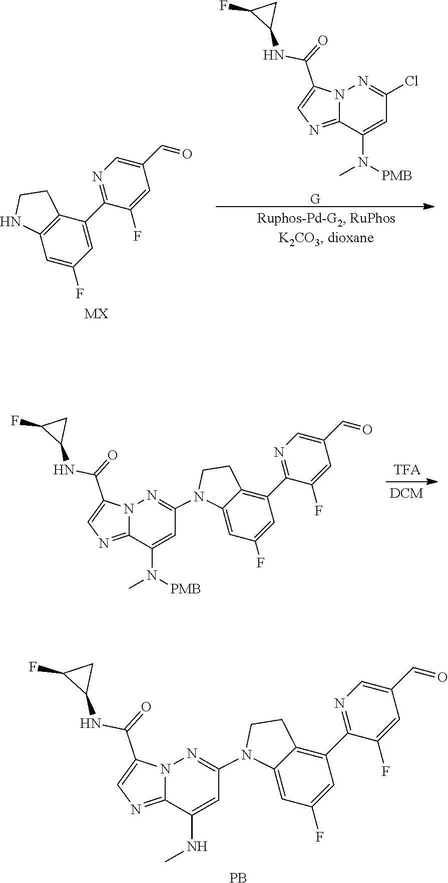

Step 1—6-(6-Fluoro-4-(3-fluoro-5-formylpyridin-2-yl)indolin-1-yl)-N-((1R,2S)-2-fluorocyclopropyl)-8-((4-methoxybenzyl)(methyl)amino)imidazo[1,2-b]pyridazine-3-carboxamide. To a stirred mixture of 6-chloro-N-[(1R,2S)-2-fluorocyclopropyl]-8-{[(4-methoxyphenyl)methyl](methyl)amino}imidazo[1,2-b]pyridazine-3-carboxamide (1229 mg, 3.043 mmol, Intermediate G) and 5-fluoro-6-(6-fluoro-2,3-dihydro-1H-indol-4-yl)pyridine-3-carbaldehyde (800 mg, 3.074 mmol, Intermediate MX) in 1,4-dioxane (1 mL) were added K₂CO₃ (1266 mg, 9.160 mmol), RuPhos (272 mg, 0.583 mmol) and RuPhos-PdCl-2nd G (454 mg, 0.584 mmol) at rt under nitrogen atmosphere. The resulting mixture was stirred for 2 h at 100° C. under nitrogen atmosphere. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 35%-55% B in 25 min; Flow rate: 80 mL/min; Detector: 220/254 nm; desired fractions were collected at 39% B) and concentrated under reduced pressure to give the title compound (600 mg, 31% yield) as a white solid. LC/MS (ESI, m/z): [(M+1)]⁺=628.2.

Step 2—6-(6-Fluoro-4-(3-fluoro-5-formylpyridin-2-yl)indolin-1-yl)-N-((1R,2S)-2-fluorocyclopropyl)-8-(methylamino)imidazo[1,2-b]pyridazine-3-carboxamide. To a solution of 6-[6-fluoro-4-(3-fluoro-5-formylpyridin-2-yl)-2,3-dihydroindol-1-yl]-N-[(1R,2S)-2-fluorocyclopropyl]-8-{[(4-methoxyphenyl)methyl](methyl)amino}imidazo[1,2-b]pyridazine-3-carboxamide (600 mg, 0.956 mmol) in DCM (6 mL) was added TFA (2 mL) dropwise at rt and the mixture was stirred for 1 h at rt. On completion, the mixture was concentrated under vacuum. The residue was purified by trituration with ethyl ether (20 mL) to afford the title compound (700 mg) as a brown solid. LC/MS (ESI, m/z): [(M+1)]⁺=508.2.

### 6-(4-(5-((5,5-Difluoro-2,7-diazaspiro[3.5]nonan-2-yl)methyl)-3-fluoropyridin-2-yl)-6-fluoroindolin-1-yl)-N-((1R,2S)-2-fluorocyclopropyl)-8-(methylamino)imidazo[1,2-b]pyridazine-3-carboxamide trifluoroacetate (Intermediate PC)

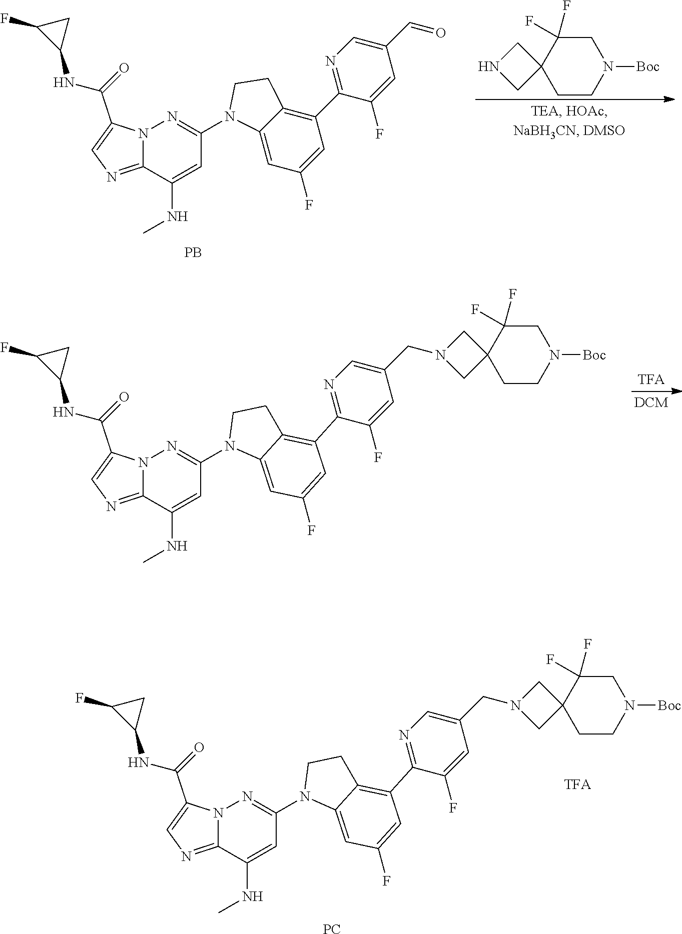

Step 1—Tert-butyl 5,5-difluoro-2-((5-fluoro-6-(6-fluoro-1-(3-(((1R,2S)-2-fluorocyclopropyl)carbamoyl)-8-(methylamino)imidazo[1,2-b]pyridazin-6-yl)indolin-4-yl)pyridin-3-yl)methyl)-2,7-diazaspiro[3.5]nonane-7-carboxylate. To a stirred mixture of 6-[6-fluoro-4-(3-fluoro-5-formylpyridin-2-yl)-2,3-dihydroindol-1-yl]-N-[(1R,2S)-2-fluorocyclopropyl]-8-(methylamino)imidazo[1,2-b]pyridazine-3-carboxamide (700 mg, 1.38 mmol, Intermediate PB) and TEA (139 mg, 1.37 mmol) in DMSO (7 mL) was added tert-butyl 5,5-difluoro-2,7-diazaspiro[3.5]nonane-7-carboxylate (434 mg, 1.66 mmol, CAS #1228631-69-3) and HOAc (248 mg, 4.13 mmol) at rt. The resulting mixture was stirred for 1 h at 50° C. To the above mixture was added NaBH₃CN (433 mg, 6.891 mmol) at rt. Then the resulting mixture was stirred for additional 1 h at 50° C. On completion, the reaction mixture was purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 35%-65% B in 30 min; Flow rate: 80 mL/min; Detector: 220/254 nm; desired fractions were collected at 55% B) and concentrated under reduced pressure to afford the title compound (500 mg, 48% yield) as a white solid. LC/MS (ESI, m/z): [(M+1)]⁺=754.3.

Step 2—6-(4-(5-((5,5-Difluoro-2,7-diazaspiro[3.5]nonan-2-yl)methyl)-3-fluoropyridin-2-yl)-6-fluoroindolin-1-yl)-N-((1R,2S)-2-fluorocyclopropyl)-8-(methylamino)imidazo[1,2-b]pyridazine-3-carboxamide trifluoroacetate. To a solution of tert-butyl 5,5-difluoro-2-({5-fluoro-6-[6-fluoro-1-(3-{[(1R,2S)-2-fluorocyclopropyl]carbamoyl}-8-(methylamino)imidazo[1,2-b]pyridazin-6-yl)-2,3-dihydroindol-4-yl]pyridin-3-yl}methyl)-2,7-diazaspiro[3.5]nonane-7-carboxylate (500 mg, 0.663 mmol) in DCM (5 mL) was added TFA (2 mL) dropwise at rt and the mixture was stirred for 1 h at rt. On completion, the resulting mixture was concentrated under vacuum. The residue was purified by trituration with ethyl ether (10 mL) to afford the title compound (425 mg, 85% yield) as a brown solid. LC/MS (ESI, m/z): [(M+1)]⁺=654.3.

### N-(3-{4-chloro-6-[(3S)-3-methoxyoxolan-3-yl]pyridin-2-yl}-1-methylpyrrolo[2,3-c]pyridin-5-yl)acetamide (Intermediate PD) and N-(3-{4-chloro-6-[(3R)-3-methoxyoxolan-3-yl]pyridin-2-yl}-1-methylpyrrolo[2,3-c]pyridin-5-yl)acetamide (Intermediate PE)

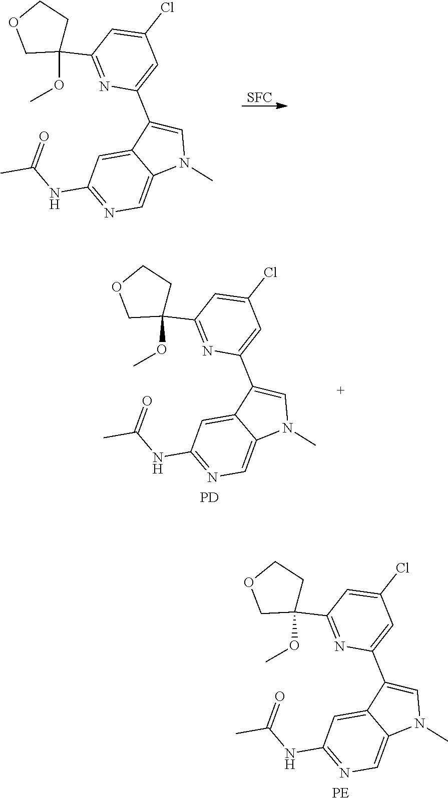

The racemic mixture of N-{3-[4-chloro-6-(3-methoxyoxolan-3-yl)pyridin-2-yl]-1-methylpyrrolo[2,3-c]pyridin-5-yl}acetamide (2.5 g, synthesized via Steps 1-5 of Intermediate NM) was separated by chiral resolution (Column: CHIRAL ART Cellulose-SB, 3*25 cm, 5 μm; Mobile Phase A: Hex(0.10% DEA)—HPLC, Mobile Phase B: MeOH: EtOH=3: 2; Flow rate: 100 mL/min; Gradient: isocratic 20% B; Back Pressure(bar): 100; Wave Length: 220 nm; RT1 (min): 16.8; RT2(min): 21; Sample Solvent: EtOH; Injection Volume: 10 mL). The fractions of slower eluting peak was collected and concentrated under vacuum to afford N-(3-{4-chloro-6-[(3S)-3-methoxyoxolan-3-yl]pyridin-2-yl}-1-methylpyrrolo[2,3-c]pyridin-5-yl)acetamide (1.14 g, 45% yield) as a white solid. The fractions of faster eluting peak was collected and concentrated under vacuum to afford N-(3-{4-chloro-6-[(3R)-3-methoxyoxolan-3-yl]pyridin-2-yl}-1-methylpyrrolo[2,3-c]pyridin-5-yl)acetamide (940 mg, 38% yield) as a white solid. LC/MS (ESI, m/z): [(M+H)]⁺=401.2 for both isomers. The absolute stereochemistry of the enantiomer was assigned arbitrarily.

### Tert-butyl 7,7-difluoro-9-{[5-(4,4,5,5-tetramethyl-1,3,2-dioxaborolan-2-yl)pyridin-2-yl]methyl}-3,9-diazaspiro[5.5]undecane-3-carboxylate (Intermediate PF)

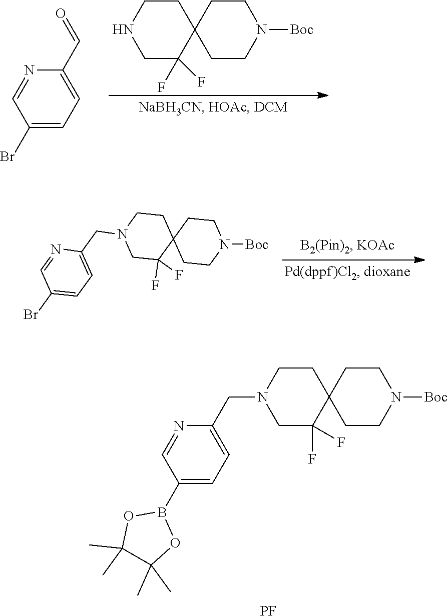 [SMILES: O=Cc1ccc(Br)cn1 ; CC(C)(C)OC(=O)N1CCC2(CCNCC2(F)F)CC1 ; CC(C)(C)OC(=O)N1CCC2(CCN(Cc3ccc(Br)cn3)CC2(F)F)CC1 ; CC(C)(C)OC(=O)N1CCC2(CCN(Cc3ccc(B4OC(C)(C)C(C)(C)O4)cn3)CC2(F)F)CC1 ; "PF"]

Step 1—Tert-butyl 9-[(5-bromopyridin-2-yl)methyl]-7,7-difluoro-3,9-diazaspiro[5.5]undecane-3-carboxylate. To a stirred mixture of Tert-butyl 7,7-difluoro-3,9-diazaspiro[5.5]undecane-3-carboxylate (500 mg, 1.72 mmol) and TEA (0.72 mL, 5.16 mmol) in DMSO (5 mL) were added 5-bromopyridine-2-carbaldehyde (320 mg, 1.72 mmol) and HOAc (0.49 mL, 8.61 mmol) at rt under nitrogen atmosphere. The resulting mixture was stirred for 30 min at 50° C. under nitrogen atmosphere. The mixture was allowed to cool down to rt. To the above mixture was added NaBH₃CN (325 mg, 5.172 mmol) at rt. The resulting mixture was stirred for an additional 1 h at 50° C. On completion, the mixture was cooled to rt and concentrated under reduced pressure. The residue was purified by reverse phase flash chromatography (Column: WelFlash™ C18-I, 20-40 μm, 320 g; Eluent A: Water (plus 10 mmol/L NH₄HCO₃); Eluent B: ACN; Gradient: 60%-85% B in 25 min; Flow rate: 80 mL/min; Detector: 220/254 nm; desired fractions were collected at 80% B) and concentrated under reduced pressure to afford the title compound (500 mg, 63% yield) as a white solid. LC/MS (ESI, m/z): [(M+H)]⁺=460.2, 462.2.

Step 2—Tert-butyl 7,7-difluoro-9-{[5-(4,4,5,5-tetramethyl-1,3,2-dioxaborolan-2-yl)pyridin-2-yl]methyl}-3,9-diazaspiro[5.5]undecane-3-carboxylate. To a stirred mixture of tert-butyl 9-[(5-bromopyridin-2-yl)methyl]-7,7-difluoro-3,9-diazaspiro[5.5]undecane-3-carboxylate (150 mg, 0.326 mmol) and KOAc (96 mg, 0.98 mmol) in dioxane (5 mL) were added BPD (165 mg, 0.650 mmol) and Pd(dpp)Cl₂ CH₂Cl₂ (27 mg, 0,033 mmol) at rt under nitrogen atmosphere. The resulting mixture was stirred for 1 h at 90° C. under nitrogen atmosphere. On completion, the mixture was cooled to rt and concentrated under reduced pressure to give the title compound (450 mg) as a black solid. LC/MS (ESI, m/z): [(M+H)]⁺=508.4.

### N-{3-[6-({1,1-difluoro-3,9-diazaspiro[5.5]undecan-3-yl}methyl)-6′-[(3R)-3-methoxyoxolan-3-yl]-[3,4′-bipyridin]-2′-yl]-1-methylpyrrolo[2,3-c]pyridin-5-yl}acetamide trifluoroacetate (Intermediate PG)

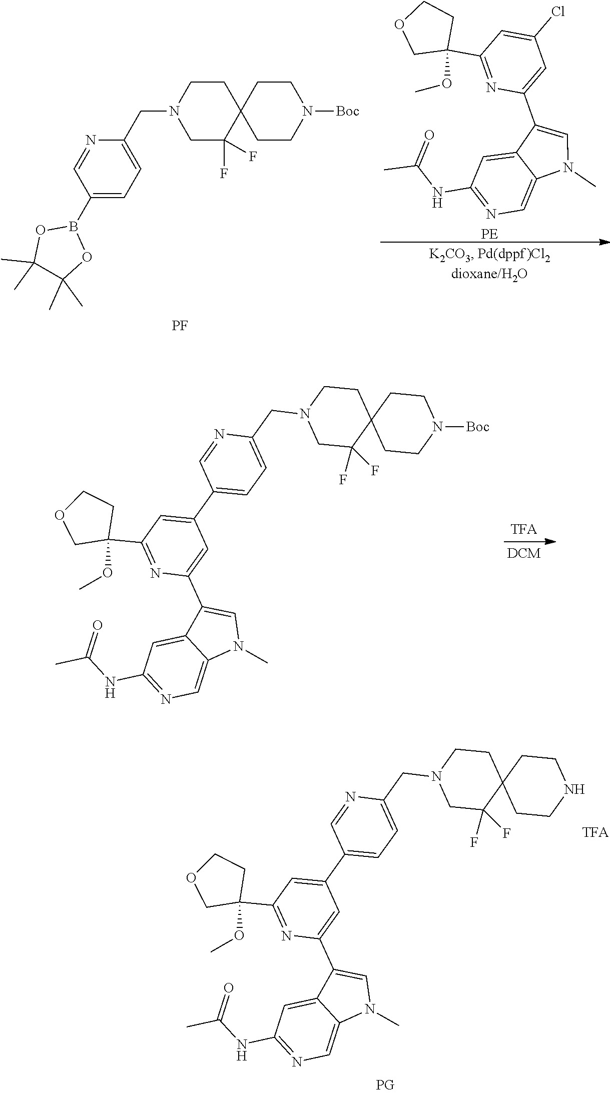

Step 1—Tert-butyl 9-[(2′-{5-acetamido-1-methylpyrrolo[2,3-c]pyridin-3-yl}-6′-[(3R)-3-methoxyoxolan-3-yl]-[3,4′-bipyridin]-6-yl)methyl]-7,7-difluoro-3,9-diazaspiro[5.5]undecane-3-carboxylate. To a stirred mixture of tert-butyl 7,7-difluoro-9-{[5-(4,4,5,5-tetramethyl-1,3,2-dioxaborolan-2-yl)pyridin-2-yl]methyl}-3,9-diazaspiro[5.5]undecane-3-carboxylate (450 mg, 0.888 mmol, Intermediate PF), K₂CO₃ (360 mg, 2.60 mmol) and H₂O (1.5 mL) in dioxane (9 mL) were added N-(3-{4-chloro-6-[(3R)-3-methoxyoxolan-3-yl]pyridin-2-yl}-1-methylpyrrolo[2,3-c]pyridin-5-yl)acetamide (282 mg, 0.702 mmol, Intermediate PE) and Pd(dppf)Cl₂CH₂Cl₂ (87 mg, 0.11 mmol) at rt under nitrogen atmosphere. The resulting mixture was stirred for 1 h at 90° C. under nitrogen atmosphere. On completion, the mixture was cooled to rt and purified by reverse phase flash chromatography (Column: WelFlash™ C18-I, 20-40 μm, 120 g; Eluent A: Water (plus 10 mmol/L NH₄HCO₃); Eluent B: ACN; Gradient: 35%-75% B in 15 min; Flow rate: 60 mL/min; Detector: 220/254 nm; desired fractions were collected at 64% B) and concentrated under reduced pressure to afford the title compound (90 mg, 17% yield) as a white solid. LC/MS (ESI, m/z): [(M+H)]⁺=746.4.

Step 2—N-{3-[6-({1,1-difluoro-3,9-diazaspiro[5.5]undecan-3-yl}methyl)-6′-[(3R)-3-methoxyoxolan-3-yl]-[³,4′-bipyridin]-2′-yl]-1-methylpyrrolo[2,3-c]pyridin-5-yl}acetamide trifluoroacetate. To a stirred solution of tert-butyl 9-[(2′-{5-acetamido-1-methylpyrrolo[2,3-c]pyridin-3-yl}-6′-[(3R)-3-methoxyoxolan-3-yl]-[3,4′-bipyridin]-6-yl)methyl]-7,7-difluoro-3,9-diazaspiro[5.5]undecane-3-carboxylate (90 mg, 0.12 mmol) in DCM (2 mL) was added TFA (1 mL) at rt under nitrogen atmosphere and the mixture was stirred for 1 h at rt under nitrogen atmosphere. On completion, the mixture was concentrated under reduced pressure. The residue was purified by trituration with Et₂O (10 mL) to afford the title compound (89 mg, 99% yield) as a light green solid. LC/MS (ESI, m/z): [(M+H)]f=646.5.

### Tert-butyl 2-((5-fluoro-6-(indolin-4-yl)pyridin-3-yl)methyl)-7-azaspiro[3.5]nonane-7-carboxylate (Intermediate PH)

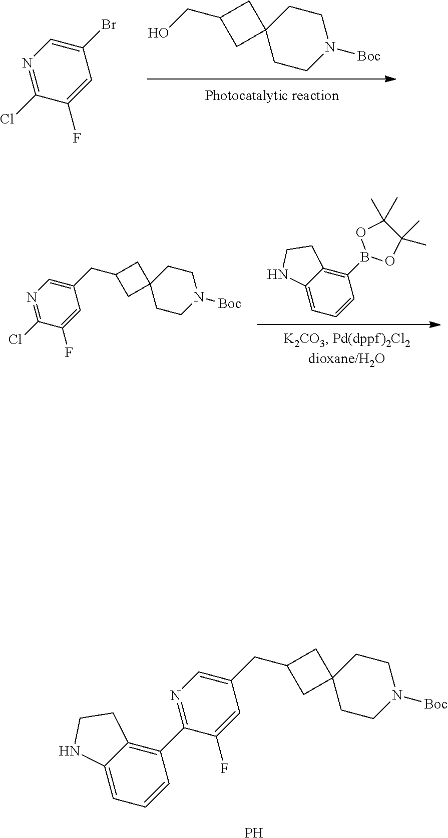

Step 1—Tert-butyl 2-[(6-chloro-5-fluoropyridin-3-yl)methyl]-7-azaspiro[3.5]nonane-7-carboxylate. An oven-dried 100 mL vial was charged with tert-butyl 2-(hydroxymethyl)-7-azaspiro[3.5]nonane-7-carboxylate (1.82 g, 7.13 mmol, CAS #1356476-27-1), 5,7-di-tert-butyl-3-phenyl-2H-1,3-benzoxazole; tetrafluoro-lambda4-borane (3.01 g, 7.60 mmol) and an X-shaped magnetic stir bar. After the vial was vacuumed and refilled with nitrogen gas twice, t-BuOMe (40 mL) was added and the reaction stirred at rt for 5 min. Then, a pyridine solution (601.43 mg, 7.603 mmol, in 8 mL t-BuOMe) was added dropwise at rt within 2 min. The resulting solution stirred at rt for 10 min. A white solid precipitated out during this time. Another oven-dried glass flask was charged with Ir[ppy]₂(dtbbpy)PF₆ (266.58 mg, 0.238 mmol). NiBr₂(dtbbpy) (115.69 mg, 0.238 mmol), quinuclidine (924.65 mg, 8.316 mmol), 5-bromo-2-chloro-3-fluoropyridine (1 g, 5 mmol) and an X-shape magnetic stir bar, then DMA (10 mL) was added to this vial under an atmosphere of nitrogen. The t-BuOMe suspension was injected through the syringe filter into the DMA solution. The resulting reaction mixture was sparged with nitrogen for 15 minutes, then irradiated under 450 nm LED for 16 h. After competition of reaction, the reaction mixture was concentrated under reduce pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 40%-55% B in 25 min; Flow rate: 80 mL/min; Detector: 220/254 nm; desired fractions were collected at 50% B) and concentrated under reduced pressure to afford the title compound (1 g, 57% yield) as a yellow solid. LC/MS (ESI, m/z): [(M-56+H)]⁺=313.1.

Step 2—Tert-butyl 2-((5-fluoro-6-(indolin-4-yl)pyridin-3-yl)methyl)-7-azaspiro[3.5]nonane-7-carboxylate. To a stirred mixture of tert-butyl 2-[(6-chloro-5-fluoropyridin-3-yl)methyl]-7-azaspiro[3.5]nonane-7-carboxylate (1 g, 3 mmol) and 4-(4,4,5,5-tetramethyl-1,3,2-dioxaborolan-2-yl)-2,3-dihydro-1H-indole (863.88 mg, 3.524 mmol) in dioxane (12 mL) and H₂O (3 mL) were added K₂CO₃ (1123.98 mg, 8.133 mmol) and Pd(dppf)Cl₂CH₂Cl₂ (220.84 mg, 0.271 mmol) at rt under nitrogen atmosphere. The resulting mixture was stirred for 2 h at 90° C. under nitrogen atmosphere. On completion, the mixture was cooled to rt and concentrated under reduced pressure. The residue was purified by silica gel column chromatography, eluted with PE/EA (5:1˜1:2), to afford the title compound (1 g, 82% yield) as a brown oil. LC/MS (ESI, m/z): [(M+H)]⁺=452.3.

### 6-[4-(5-{7-Azaspiro[3.5]nonan-2-ylmethyl}-3-fluoropyridin-2-yl)-2,3-dihydroindol-1-yl]-N-[(1R,2R)-2-methoxycyclobutyl]-8-(methylamino)imidazo[1,2-b]pyridazine-3-carboxamide trifluoroacetate (Intermediate PI)

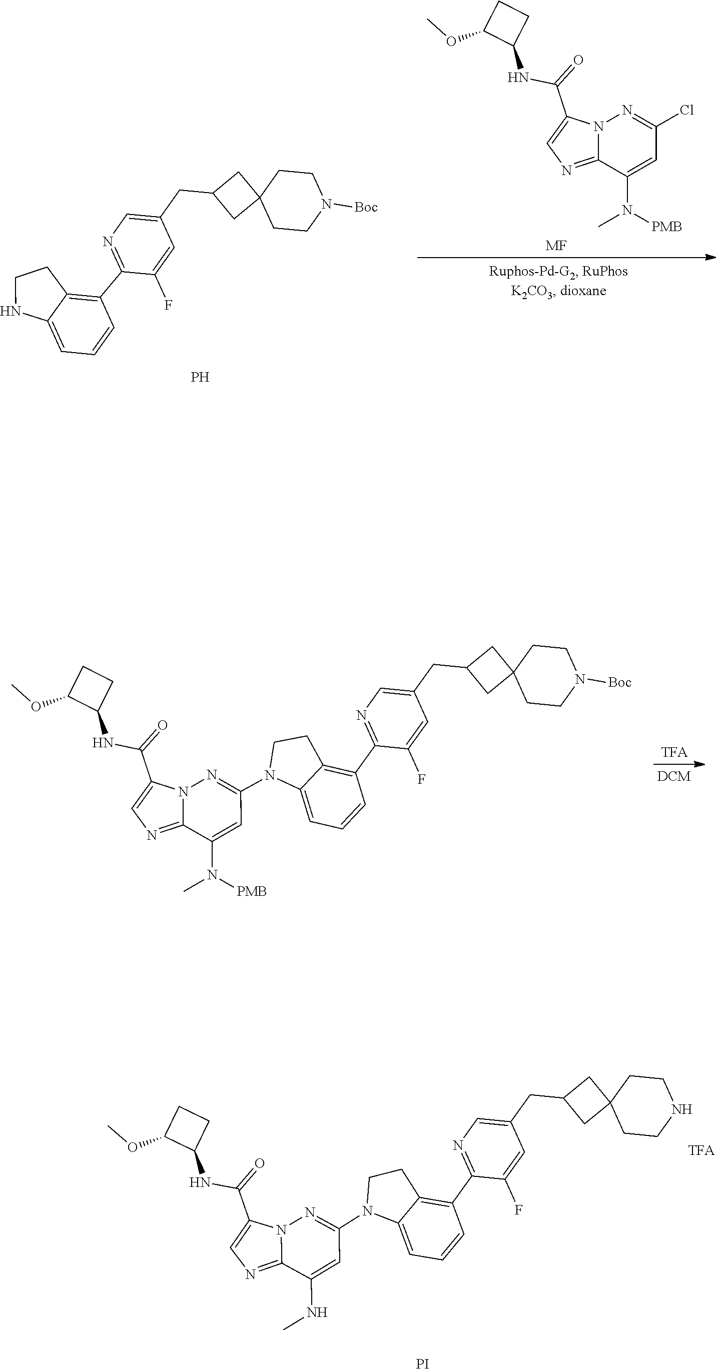

Step 1—Tert-butyl 2-({5-fluoro-6-[1-(3-{[(1R,2R)-2-methoxycyclobutyl]carbamoyl}-8-{1[(4-methoxyphenyl)methyl](methyl)amino}imidazo[1,2-b]pyridazin-6-yl)-2,3-dihydroindol-4-yl]pyridin-3-yl}methyl)-7-azaspiro[3.5]nonane-7-carboxylate. To a stirred mixture of tert-butyl 2-{[6-(2,3-dihydro-1H-indol-4-yl)-5-fluoropyridin-3-yl]methyl}-7-azaspiro[3.5]nonane-7-carboxylate (1 g, 2 mmol, Intermediate PH) and 6-chloro-N-[(1R,2R)-2-methoxycyclobutyl]-8-{[(4-methoxyphenyl)methyl](methyl)amino}imidazo[1,2-b]pyridazine-3-carboxamide (1.14 g, 2.66 mmol, Intermediate MF) in dioxane (12 mL) were added RuPhos (206.67 mg, 0.443 mmol), RuPhos-PdCl-2nd G (172.22 mg, 0.221 mmol) and K₂CO₃ (918.13 mg, 6.642 mmol) at it under nitrogen atmosphere. The resulting mixture was stirred for 2 h at 100° C. under nitrogen atmosphere. On completion, the mixture was cooled to rt and concentrated under reduced pressure. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 75%-95% B in 25 min; Flow rate: 80 mL/min; Detector: 220/254 nm; desired fractions were collected at 100% B) and concentrated under reduced pressure to afford the title compound (1.5 g, 80% yield) as a brown yellow solid. LC/MS (ESI, m/z): [(M+H)]⁺=845.4.

Step 2—6-[4-(5-{7-Azaspiro[3.5]nonan-2-ylmethyl}-3-fluoropyridin-2-yl)-2,3-dihydroindol-1-yl]-N-[(1R,2R)-2-methoxycyclobutyl]-8-(methylamino)imidazo[1,2-b]pyridazine-3-carboxamide trifluoroacetate. To a stirred solution of tert-butyl 2-({5-fluoro-6-[1-(3-{[(1R,2R)-2-methoxycyclobutyl]carbamoyl}-8-{[(4-methoxyphenyl)methyl](methyl)amino}imidazo[1,2-b]pyridazin-6-yl)-2,3-dihydroindol-4-yl]pyridin-3-yl}methyl)-7-azaspiro[3.5]nonane-7-carboxylate (1.5 g, 1.8 mmol) in DCM (10 mL) were added TFA (5 mL) at rt and mixture was stirred for 30 min at rt. On completion, the mixture was concentrated under reduced pressure. The residue was purified by trituration with Et₂O (30 mL) to afford the title compound (1.6 g) as yellow solid. LC/MS (ESI, m/z): [(M+H)]⁺=625.3.

### 6-(4-(5-((R)-1-(5,5-difluoro-2,7-diazaspiro[3.5]nonan-2-yl)ethyl)-3-fluoropyridin-2-yl)indolin-1-yl)-N-((1R,2R)-2-methoxycyclobutyl)-8-(methylamino)imidazo[1,2-b]pyridazine-3-carboxamide trifluoroacetate (Intermediate PJ)

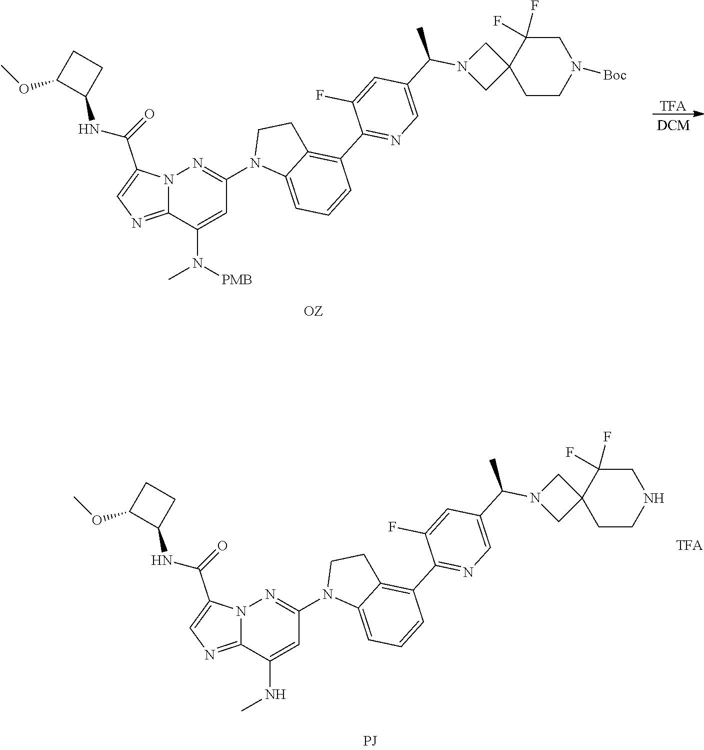

A solution of tert-butyl 5,5-difluoro-2-((R)-1-(5-fluoro-6-(1-(8-((4-methoxybenzyl)(methyl)amino)-3-(((1R,2R)-2-methoxycyclobutyl)carbamoyl)imidazo[1,2-b]pyridazin-6-yl)indolin-4-yl)pyridin-3-yl)ethyl)-2,7-diazaspiro[3.5]nonane-7-carboxylate (110 mg, 0.123 mmol, Intermediate OZ) in DCM (5 mL) and TFA (1 mL) was stirred for 1 h at rt. On completion, the resulting mixture was concentrated under reduced pressure. The resulting mixture was diluted with diethyl ether (20 mL). The precipitated solids were collected by filtration and washed with diethyl ether (2×10 mL) to give the title compound (100 mg, TFA salt) as an off-white solid. LC/MS (ESI, m/z): [(M+1)]⁺=676.3.

### 5-Chloro-4-(2,4-dioxotetrahydropyrimidin-1(2H)-yl)-6-methylpicolinic acid (Intermediate PK)

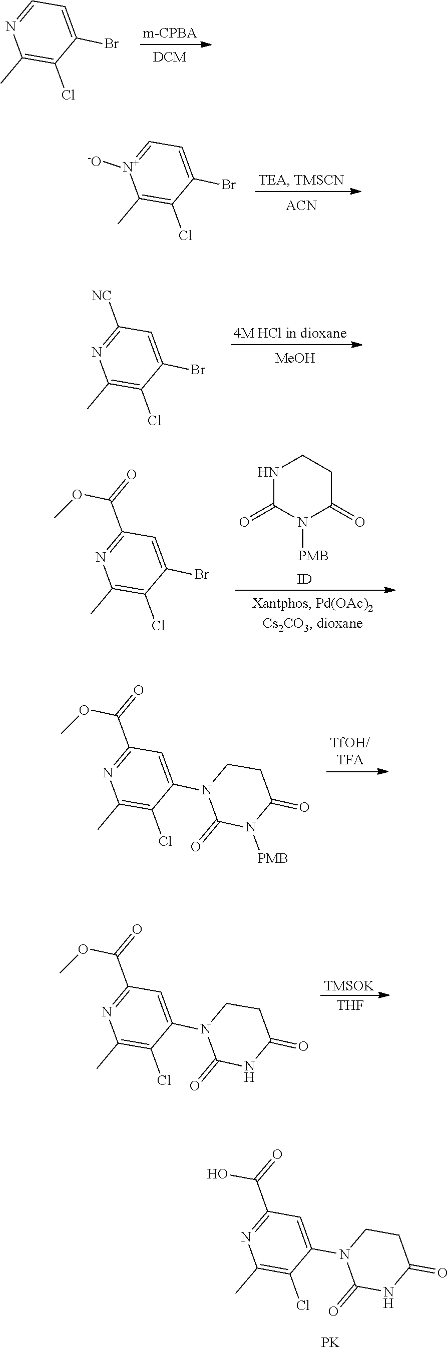

Step 1—4-Bromo-3-chloro-2-methylpyridine 1-oxide. To a stirred solution of 4-bromo-3-chloro-2-methylpyridine (5.00 g, 24.2 mmol, CAS #1188140-52-4) in DCM (50 mL) was added m-CPBA (6.27 g, 36.3 mmol) in portions at 0° C. under nitrogen atmosphere. The resulting mixture was stirred for 1 h at rt under nitrogen atmosphere. On completion, the mixture was concentrated under reduced pressure. The resulting mixture was diluted with H₂O (300 mL) and the mixture was extracted with EtOAc (2×300 mL). The combined organic layers were dried over anhydrous Na₂SO₄ filtered, and the filtrate was concentrated under reduced pressure. The residue was purified by silica gel column chromatography, eluted with DCM/MeOH (10:1), to afford the title compound (5.20 g, 97%) as a white solid. LC/MS (ESI, m/z): [(M+H)]⁺=222.0, 224.0.

Step 2—4-Bromo-5-chloro-6-methylpicolinonitrile. To a stirred solution of 4-bromo-3-chloro-2-methylpyridine 1-oxide (5.00 g, 22.5 mmol) in ACN (60 mL) was added TEA (13.65 g, 134.9 mmol) at rt. To the above mixture was added TMSCN (8.92 g, 89.9 mmol) at 0° C. The resulting mixture was stirred for an additional 48 h at 100° C. On completion, the mixture was cooled to rt and concentrated under reduced pressure. The residue was purified by silica gel column chromatography, eluted with PE/EA (5:1), to afford the title compound (2.80 g, 54% yield) as a yellow solid. ¹H NMR (400 MHz, DMSO-d₆) δ 8.46 (s, 1H), 2.66 (s, 3H).

Step 3—Methyl 4-bromo-5-chloro-6-methylpicolinate. To a stirred solution of 4-bromo-5-chloro-6-methylpicolinonitrile (2.80 g, 12.1 mmol) in MeOH (10 mL) was added 4 M HCl (gas) in 1,4-dioxane (10 mL) at rt. The resulting mixture was stirred for 1 h at 80° C. On completion, the mixture was cooled to rt and concentrated under reduced pressure. The residue was purified by silica gel column chromatography, eluted with PE/EA (5:1), to afford the title compound (1.70 g, 53% yield) as a white solid. ¹H NMR (300 MHz, DMSO-d₆) δ 8.04 (s, 1H), 3.86 (s, 3H), 2.62 (s, 3H).

Step 4—Methyl 5-chloro-4-(3-(4-methoxybenzyl)-2,4-dioxotetrahydropyrimidin-1(2H)-yl)-6-methylpicolinate. To a stirred solution of methyl 4-bromo-5-chloro-6-methylpicolinate (1.00 g, 3.78 mmol) and 3-(4-methoxybenzyl)dihydropyrimidine-2,4(1H,3H)-dione (885.65 mg, 3.78 mmol, Intermediate ID) in 1,4-dioxane (10 mL) were added XantPhos (437.53 mg, 0.76 mmol), Cs₂CO₃ (3.70 g, 11.34 mmol) and Pd(OAc)₂ (169.76 mg, 0.76 mmol) in turns at rt under nitrogen atmosphere. The resulting mixture was stirred for an additional 2 h at 100° C. On completion, the mixture was cooled to rt and concentrated under reduced pressure. The residue was purified by silica gel column chromatography, eluted with PE/EA (1:1), to afford the title compound (1.00 g, 63% yield) as a yellow solid. LC/MS (ESI, m/z): [(M+H)]⁺=418.2.

Step 5—Methyl 5-chloro-4-(2,4-dioxotetrahydropyrimidin-1(2H)-yl)-6-methylpicolinate. To a stirred solution of methyl 5-chloro-4-(3-(4-methoxybenzyl)-2,4-dioxotetrahydropyrimidin-1(2H)-yl)-6-methylpicolinate (1.00 g, 2.39 mmol) in TFA (10 mL) was added trifluoromethanesulfonic acid (0.5 mL) dropwise at rt under air atmosphere. The resulting mixture was stirred for 1 h at 70° C. under air atmosphere. On completion, the mixture was cooled to rt and concentrated under reduced pressure. The mixture was basified to pH 8 with saturated ammonium bicarbonate aqueous solution. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 10%-30% B in 20 min; Flow rate: 60 mL/min; Detector: 220/254 nm; desired fractions were collected at 20% B) and concentrated under reduced pressure to afford the title compound (400 mg, 56% yield) as a white solid. LC/MS (ESI, m/z): [(M+H)]⁺=298.0.

Step 6—5-Chloro-4-(2,4-dioxotetrahydropyrimidin-1(2H)-yl)-6-methylpicolinic acid. To a stirred solution of methyl 5-chloro-4-(2,4-dioxotetrahydropyrimidin-1(2H)-yl)-6-methylpicolinate (400 mg, 1.34 mmol) in THF (20 mL) was added trimethyl(potassiooxy)silane (861.85 mg, 6.72 mmol) at rt under air atmosphere and the mixture was stirred for 1 h at rt. On completion, the mixture was acidified to pH 4 with 2M HCl (aq.). The precipitated solids were collected by filtration and washed with H₂O (3×10 mL). The resulting solid was dried under reduced pressure to afford the title compound (290 mg, 76% yield) as a white solid. LC/MS (ESI, m/z): [(M+H)]⁺=284.0.

### 3-(6-{4-[(4S)-3,3-difluoropiperidin-4-yl]piperazin-1-yl}-1-methylindazol-3-yl)piperidine-2,6-dione trifluoroacetate (Intermediate PL)

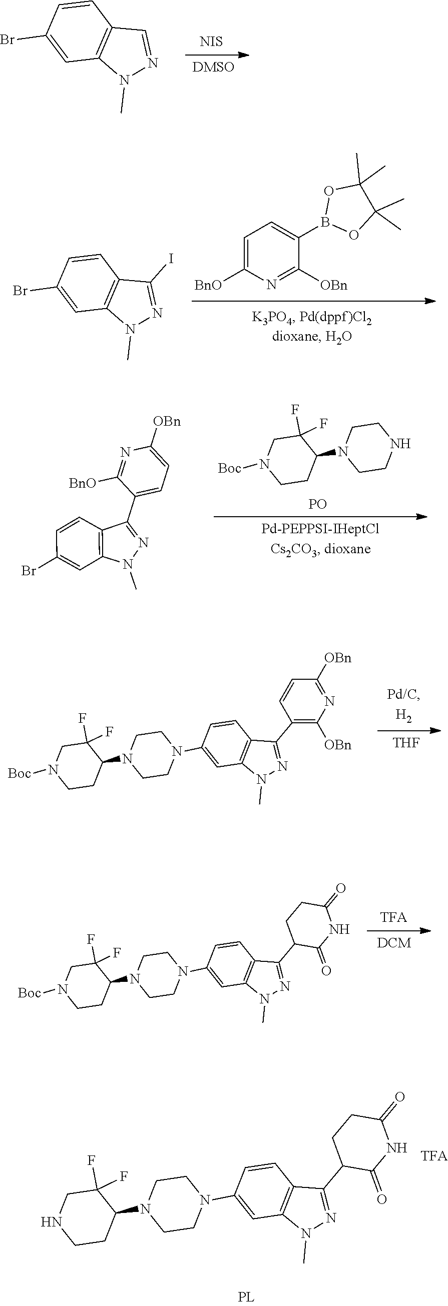

Step 1—6-Bromo-3-iodo-1-methylindazole. To a stirred mixture of 6-bromo-1-methylindazole (1 g, 5 mmol) in DMSO (10 mL) was added NIS (1.17 g, 5.21 mmol) at rt. The resulting mixture was stirred for an additional 16 h at 90° C. under nitrogen atmosphere. On completion, the mixture was cooled to rt and diluted with water (100 mL). The resulting mixture was extracted with EtOAc (3×50 mL) and the combined organic layers were washed with brine (1×100 mL), dried over anhydrous Na₂SO₄, filtered, and the filtrate was concentrated under reduced pressure. The residue was purified by silica gel column chromatography, eluted with PE/EA (2:1˜1:1), to afford the title compound (710 mg, 45% yield) as a yellow solid. LC/MS (ESI, m/z): [(M+H)]⁺=336.8, 338.8.

Step 2—3-[2,6-Bis(benzyloxy)pyridin-3-yl]-6-bromo-1-methylindazole. To a stirred mixture of 6-bromo-3-iodo-1-methylindazole (700 mg, 2.08 mmol) and 2,6-bis(benzyloxy)-3-(4,4,5,5-tetramethyl-1,3,2-dioxaborolan-2-yl)pyridine (866.92 mg, 2.077 mmol, CAS #2152673-80-6) in dioxane (6 mL) and H₂O (1.2 mL) were added K₃PO₄ (1322.88 mg, 6.231 mmol) and Pd(dppnfCl₂·CH₂Cl, (169.23 mg, 0.208 mmol) at rt under nitrogen atmosphere. The resulting mixture was stirred for an additional 1 h at 100° C. On completion, the mixture was cooled to rt and concentrated under reduced pressure. The residue was purified by silica gel column chromatography, eluted with PE/EA (2:1˜1:1), to afford the title compound (640 mg, 62% yield) as a yellow solid. LC/MS (ESI, m/z): [(M+H)]⁺=500.1, 502.1.

Step 3—Tert-butyl (4S)-4-(4-{3-[2,6-bis(benzyloxy)pyridin-3-yl]-1-methylindazol-6-yl}piperazin-1-yl)-3,3-difluoropiperidine-1-carboxylate. To a stirred mixture of 3-[2,6-bis(benzyloxy)pyridin-3-yl]-6-bromo-1-methylindazole (620 mg, 1.239 mmol) and tert-butyl (4S)-3,3-difluoro-4-(piperazin-1-yl)piperidine-1-carboxylate (378.36 mg, 1.239 mmol, Intermediate PO) in dioxane (7 mL) were added Cs₂CO₃(1211.09 mg, 3.717 mmol) and Pd-PEPPSI-IHeptCl 3-chloropyridine (120.65 mg, 0.124 mmol) at rt under nitrogen atmosphere. The resulting mixture was stirred for an additional 3 h at 100° C. On completion, the mixture was cooled to rt and concentrated under reduced pressure. The residue was purified by silica gel column chromatography, eluted with PE/EA (2:1˜1:1), to afford the title compound (420 mg, 47% yield) as a yellow solid. LC/MS (ESI, m/z): [(M+H)]⁺=725.2.

Step 4—Tert-butyl (4S)-4-{4-[3-(2,6-dioxopiperidin-3-yl)-1-methylindazol-6-yl]piperazin-1-yl}-3,3-difluoropiperidine-1-carboxylate. To a stirred mixture of tert-butyl (4S)-4-(4-{3-[2,6-bis(benzyloxy)pyridin-3-yl]-1-methylindazol-6-yl}piperazin-1-yl)-3,3-difluoropiperidine-1-carboxylate (400 mg, 0.552 mmol) in THF (10 mL) was added Pd/C (571.41 mg, 5.371 mmol) at rt under nitrogen atmosphere. The resulting mixture was purged with hydrogen three times and stirred for 1 h at rt under hydrogen atmosphere. On completion, the mixture was filtered, and the filter cake was washed with THF (3×10 mL). The filtrate was concentrated under reduced pressure to give the title compound (290 mg, 96% yield) as a yellow green solid. LC/MS (ESI, m/z): [(M+H)]⁺=547.2.

Step 5—3-(6-{4-[(4S)-3,3-difluoropiperidin-4-yl]piperazin-1-yl}-1-methylindazol-3-yl)piperidine-2,6-dione trifluoroacetate. To a stirred solution of tert-butyl (4S)-4-{4-[3-(2,6-dioxopiperidin-3-yl)-1-methylindazol-6-yl]piperazin-1-yl}-3,3-difluoropiperidine-1-carboxylate (290 mg, 0.531 mmol) in DCM (3 mL) was added TFA (1 mL) at rt under nitrogen atmosphere and the mixture was stirred for 1 h at rt. On completion, the mixture was concentrated under reduced pressure. The residue was purified by trituration with ethyl ether (30 mL) to give the title compound (290 mg) as a yellow solid. LC/MS (ESI, m/z): [(M+H)]⁺=447.1.

### 3-(6-{4-[(4S)-3,3-difluoropiperidin-4-yl]piperazin-1-yl}-5-fluoro-1-methylindazol-3-yl)piperidine-2,6-dione trifluoroacetate (Intermediate PM)

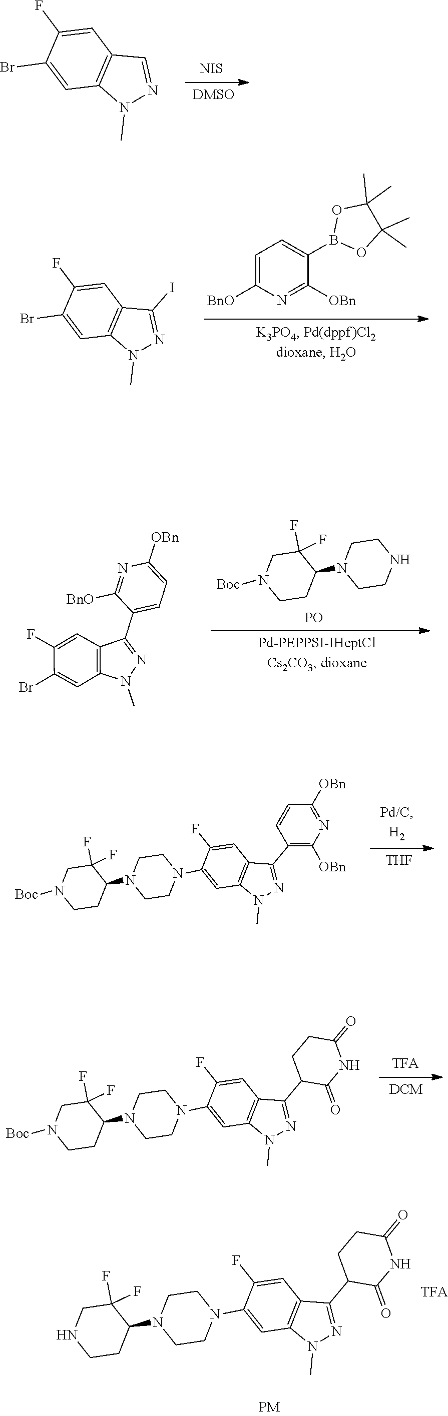

Step 1—6-Bromo-5-fluoro-3-iodo-1-methylindazole. To a stirred solution of 6-bromo-5-fluoro-1-methylindazole (1 g, 4 mmol, CAS #128734-86-8) in DMSO (15 mL) was added NIS (1.08 g, 4.80 mmol) at rt under nitrogen atmosphere. The resulting mixture was stirred for an additional 16 h at 90° C. On completion, the mixture was cooled to rt and diluted with water (50 mL). The resulting mixture was extracted with EtOAc (3×50 mL). The combined organic layers were washed with brine (1×100 mL), dried over anhydrous Na₂SO₄, filtered, and the filtrate was concentrated under reduced pressure. The residue was purified by silica gel column chromatography, eluted with PE/EA (10:1), to afford the title compound (520 mg, 34% yield) as a white solid. LC/MS (ESI, m/z): [(M+H)]⁺=355.1, 357.1.

Step 2—3-[2,6-Bis(benzyloxy)pyridin-3-yl]-6-bromo-5-fluoro-1-methylindazole. To a stirred mixture of 6-bromo-5-fluoro-3-iodo-1-methylindazole (520 mg, 1.47 mmol) and 2,6-bis(benzyloxy)-3-(4,4,5,5-tetramethyl-1,3,2-dioxaborolan-2-yl)pyridine (611.36 mg, 1.465 mmol, CAS #2152673-80-6) in dioxane (10 mL) and H₂O (2 mL) were added K₃PO₄ (932.90 mg, 4.395 mmol) and Pd(dppf)Cl₂CH₂Cl₂ (119.34 mg, 0.147 mmol) at rt under nitrogen atmosphere. The resulting mixture was stirred for an additional 1 h at 100° C. On completion, the mixture was cooled and concentrated under reduced pressure. The residue was purified by silica gel column chromatography, eluted with PE/EA (5:1), to afford the title compound (480 mg, 63% yield) as a yellow solid. LC/MS (ESI, m/z): [(M+H)]⁺=518.2, 520.2.

Step 3—Tert-butyl (4S)-4-(4-{3-[2,6-bis(benzyloxy)pyridin-3-yl]-5-fluoro-1-methylindazol-6-yl}piperazin-1-yl)-3,3-difluoropiperidine-1-carboxylate. To a stirred mixture of 3-[2,6-bis(benzyloxy)pyridin-3-yl]-6-bromo-5-fluoro-1-methylindazole (480 mg, 0.926 mmol) and tert-butyl (4S)-3,3-difluoro-4-(piperazin-1-yl)piperidine-1-carboxylate (282.76 mg, 0.926 mmol, Intermediate PO) in dioxane (10 mL) were added Cs₂CO₃ (905.08 mg, 2.778 mmol) and Pd-PEPPSI-IHeptCl 3-chloropyridine (90.17 mg, 0.093 mmol) at rt under nitrogen atmosphere. The resulting mixture was stirred for an additional 2 h at 100° C. On completion, the mixture was cooled to rt and concentrated under reduced pressure. The residue was purified by silica gel column chromatography, eluted with PE/EA (1:2), to afford the title compound (500 mg, 73% yield) as a white solid. LC/MS (ESI, m/z): [(M+H)]⁺=743.2.

Step 4—Tert-butyl (4S)-4-{4-[3-(2,6-dioxopiperidin-3-yl)-5-fluoro-1-methylindazol-6-yl]piperazin-1-yl}-3,3-difluoropiperidine-1-carboxylate. To a stirred mixture of tert-butyl (4S)-4-(4-{3-[2,6-bis(benzyloxy)pyridin-3-yl]-5-fluoro-1-methylindazol-6-yl}piperazin-1-yl)-3,3-difluoropiperidine-1-carboxylate (500 mg, 0.673 mmol) in THF (10 mL) was added Pd/C (300 mg) at rt under nitrogen atmosphere. The resulting mixture was purged with hydrogen three times and stirred for 1 h at rt under hydrogen atmosphere. On completion, the mixture was filtered, and the filter cake was washed with THF (3×30 mL). The filtrate was concentrated under reduced pressure to give the title compound (350 mg, 92% yield) as a white solid. LC/MS (ESI, m/z): [(M+H)]⁺=565.2.

Step 5—3-(6-{4-[(4S)-3,3-difluoropiperidin-4-yl]piperazin-1-yl}-5-fluoro-1-methylindazol-3-yl)piperidine-2,6-dione trifluoroacetate. To a stirred solution of tert-butyl (4S)-4-{4-[3-(2,6-dioxopiperidin-3-yl)-5-fluoro-1-methylindazol-6-yl]piperazin-1-yl}-3,3-difluoropiperidine-1-carboxylate (150 mg, 0.266 mmol) in DCM (2 mL) was added TFA (1 mL) dropwise at rt under nitrogen atmosphere and the mixture was stirred for 1 h at rt. On completion, the mixture was concentrated under reduced pressure. The residue was purified by trituration with Et₂O (30 mL) to give the title compound (150 mg, 98% yield) as a white solid. LC/MS (ESI, m/z): [(M+H)]⁺=465.2.

### Tert-butyl (R)-3,3-difluoro-4-(piperazin-1-yl)piperidine-1-carboxylate (Intermediate PN) and tert-butyl (S)-3,3-difluoro-4-(piperazin-1-yl)piperidine-1-carboxylate (Intermediate PO)

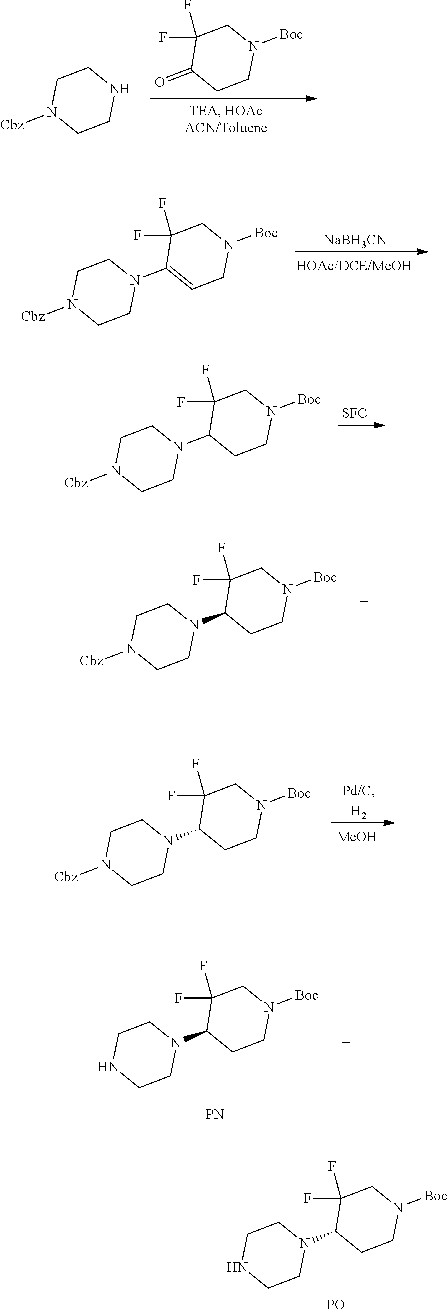

Step 1—Benzyl 4-(1-(tert-butoxycarbonyl)-3,3-difluoro-1,2,3,6-tetrahydropyridin-4-yl)piperazine-1-carboxylate. To a stirred mixture of benzyl piperazine-1-carboxylate (32.77 g, 148.8 mmol) in MeCN (250 mL) and toluene (250 mL) were added Et₃N (22.58 g, 223.2 mmol) and tert-butyl 3,3-difluoro-4-oxopiperidine-1-carboxylate (35.00 g, 148.8 mmol) and HOAc (26.81 g, 446.4 mmol) at rt. The resulting mixture was stirred overnight at 110° C. On completion, the mixture was cooled to rt and basified to pH 8 with saturated NaHCO₃ (aq.). The resulting mixture was diluted with water (300 mL) and extracted with EtOAc (3×300 mL). The combined organic layers were washed with brine (1×500 mL), and dried over anhydrous Na₂SO₄. After filtration, the filtrate was concentrated under reduced pressure to afford the title compound (59 g) as a brown oil. LC/MS (ESI, m/z): [(M+1)]=438.2.

Step 2—Benzyl 4-(1-(tert-butoxycarbonyl)-3,3-difluoropiperidin-4-yl)piperazine-1-carboxylate. To a stirred mixture of benzyl 4-(1-(tert-butoxycarbonyl)-3,3-difluoro-1,2,3,6-tetrahydropyridin-4-yl)piperazine-1-carboxylate (50.00 g, 114.3 mmol) in DCE (250 mL), MeOH (250 mL) and HOAc (25 mL) was added NaBH₃CN (35.91 g, 571.44 mmol) in portions at rt and the mixture was stirred for 1 h at rt. On completion, the mixture was concentrated under reduced pressure. The resulting mixture was diluted with water (300 mL) and extracted with EtOAc (3×200 mL). The combined organic layers were washed with brine (1×500 mL), dried over anhydrous Na₂SO₄, filtered, and the filtrate was concentrated under reduced pressure. The residue was purified by silica gel column chromatography, eluted with PE/EA (3:1), to afford the title compound (27 g, 53%) as a colorless oil. LC/MS (ESI, m/z): [(M+1)]=440.2.

Step 3—Benzyl (R)-4-(1-(tert-butoxycarbonyl)-3,3-difluoropiperidin-4-yl)piperazine-1-carboxylate and benzyl (S)-4-(1-(tert-butoxycarbonyl)-3,3-difluoropiperidin-4-yl)piperazine-1-carboxylate. The racemic mixture of benzyl 4-(1-(tert-butoxycarbonyl)-3,3-difluoropiperidin-4-yl)piperazine-1-carboxylate (27.00 g) was separated by Prep-chiral-HPLC (Column: CHIRALPAK IG, 7*25 cm, 10 μm; Mobile Phase A: CO₂, Mobile Phase B: MeOH: ACN=1: 1; Flow rate: 250 mL/min; Gradient: isocratic 35% B; Column Temperature(° C.): 35; Back Pressure(bar): 100; Wave Length: 220 nm; RT1 (min): 5.2; RT2(min): 8.2; Sample Solvent: MeOH; Injection Volume: 2 mL; Number Of Runs: 60). The fractions of faster eluting peak was collected and concentrated under vacuum to afford benzyl (R)-4-(1-(tert-butoxycarbonyl)-3,3-difluoropiperidin-4-yl)piperazine-1-carboxylate (9.3 g) as a white solid. LC/MS (ESI, m/z): [(M+1)]⁺=440.2. The fractions of slower eluting peak was collected and concentrated under vacuum to afford benzyl (S)-4-(1-(tert-butoxycarbonyl)-3,3-difluoropiperidin-4-yl)piperazine-1-carboxylate (10 g) as a white solid. LC/MS (ESI, m/z): [(M+1)]=440.2. The absolute stereochemistry of the enantiomers was assigned arbitrarily.

Step 4A—Tert-butyl (R)-3,3-difluoro-4-(piperazin-1-yl)piperidine-1-carboxylate. To a solution of benzyl (R)-4-(1-(tert-butoxycarbonyl)-3,3-difluoropiperidin-4-yl)piperazine-1-carboxylate (9.30 g, 21.2 mmol) in MeOH (200 mL) was added Pd/C (4.49 g, 42.4 mmol) under nitrogen atmosphere. The reaction system was degassed under vacuum and purged with H₂ several times, then it was hydrogenated under H₂ balloon (˜1 atm) at rt for 3 h. After completion of the reaction, Pd/C was filtered off through celite and the filter cake was washed with MeOH (3×100 mL). The corresponding filtrate was concentrated under reduced pressure to afford the title compound (7.1 g) as a white solid. LC/MS (ESI, m/z): [(M+1)]⁺=306.2.

Step 4B: Tert-butyl (S)-3,3-difluoro-4-(piperazin-1-yl)piperidine-1-carboxylate. To a solution of benzyl (S)-4-(1-(tert-butoxycarbonyl)-3,3-difluoropiperidin-4-yl)piperazine-1-carboxylate (10.00 g, 22.78 mmol) in MeOH (200 mL) was added Pd/C (4.83 g, 45.6 mmol) under nitrogen atmosphere. The reaction system was degassed under vacuum and purged with H₂ several times, then it was hydrogenated under H₂ balloon (˜1 atm) at rt for 3 h. After completion of the reaction, Pd/C was filtered off through celite and the filter cake was washed with MeOH (3×100 mL). The corresponding filtrate was concentrated under reduced pressure to afford the title compound (7.8 g) as a white solid. LC/MS (ESI, m/z): [(M+1)]⁺=306.2.

### 3-(5-Bromo-2-chlorophenyl)piperidine-2,6-dione (Intermediate PP)

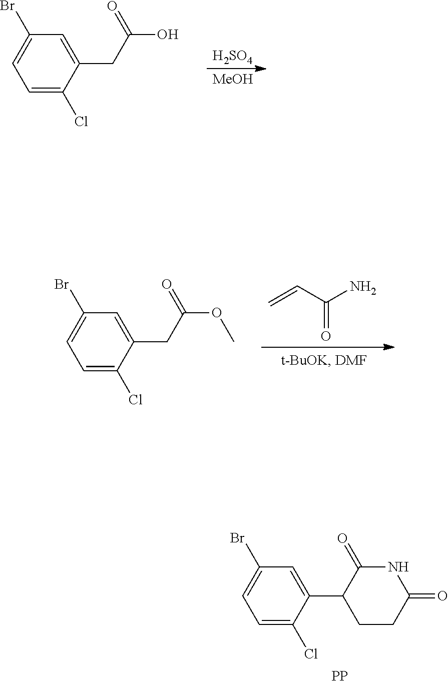

Step 1—Methyl 2-(5-bromo-2-chlorophenyl)acetate. To a stirred solution of methyl 2-(5-bromo-2-chlorophenyl)acetate (24 g, 91 mmol) in MeOH (300 mL) was added conc.H2SO4 (5.85 mL, 91.1 mmol) dropwise at rt under nitrogen atmosphere. The resulting mixture was stirred for 1 h at 70° C. under nitrogen atmosphere. On completion, the mixture was cooled to rt and concentrated under reduced pressure. The residue was purified by silica gel column chromatography, eluted with PE/EA (5:1), to afford (23 g, 96% yield) as a yellow oil. LC/MS (ESI, m/z): [(M+H)]⁺=263.0, 265.0.

Step 2—3-(5-Bromo-2-chlorophenyl)piperidine-2,6-dione. To a stirred solution of methyl 2-(5-bromo-2-chlorophenyl)acetate (8.70 g, 33.0 mmol) and polyacrylamide (2.35 g, 33.0 mmol) in DMF (100 mL) was added t-BuOK (5.56 g, 49.5 mmol) at rt under nitrogen atmosphere and the mixture was stirred for 2 h at rt under nitrogen atmosphere. On completion, the reaction mixture was concentrated under reduced pressure. The residue was purified by reverse flash chromatography (column, C18 silica gel; mobile phase A: water (10 mmol/L NH₄HCO₃), mobile phase B: ACN, 50% to 80% gradient in 30 min; detector, UV 254 nm; the fractions were collected at 70% and concentrated under reduced pressure) to afford the title compound (5.50 g, 55% yield) as a white solid. LC/MS (ESI, m/z): [(M+H)]⁺=302.0, 305.0.

### 6-(4-(5-((2,8-Diazaspiro[4.5]decan-2-yl)methyl)pyridin-2-yl)indolin-1-yl)-N-((1R,2S)-2-fluorocyclopropyl)-8-(methylamino)imidazo[1,2-b]pyridazine-3-carboxamide 2,2,2-trifluoroacetate (Intermediate PQ)

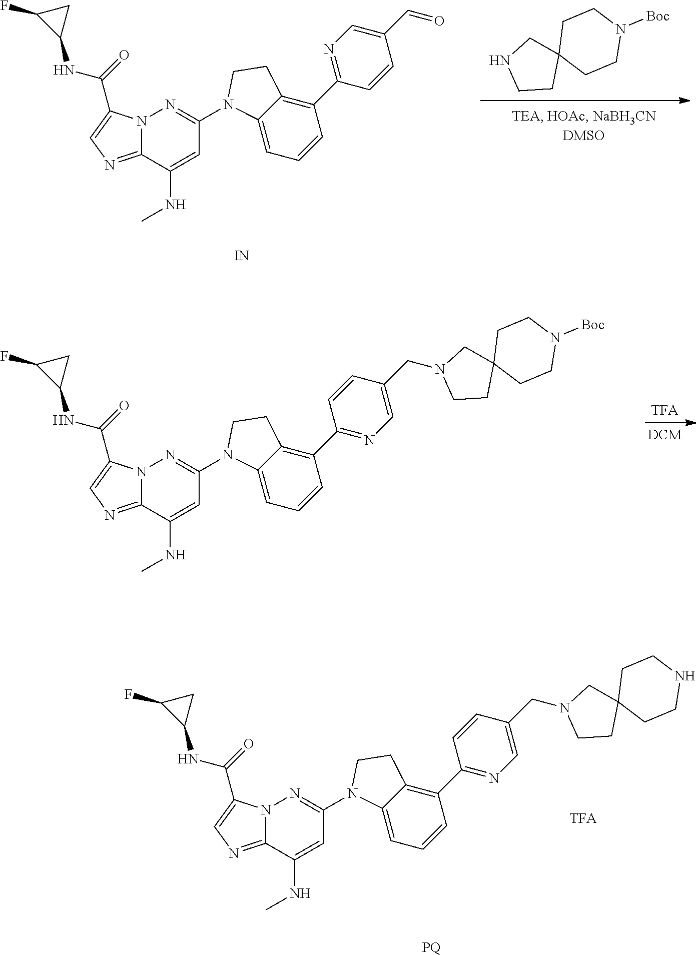

Step 1—Tert-butyl 2-((6-(1-(3-(((1R,2S)-2-fluorocyclopropyl)carbamoyl)-8-(methylamino)imidazo[1,2-b]pyridazin-6-yl)indolin-4-yl)pyridin-3-yl)methyl)-2,8-diazaspiro[4.5]decane-8-carboxylate. To a stirred solution of tert-butyl 2,8-diazaspiro[4.5]decane-8-carboxylate (168 mg, 0.70 mmol, CAS #236406-39-6) and TEA (64 mg, 0.63 mmol) in DMSO (4 mL) were added N-((1R,2S)-2-fluorocyclopropyl)-6-(4-(5-formylpyridin-2-yl)indolin-1-yl)-8-(methylamino)imidazo[1,2-b]pyridazine-3-carboxamide (300 mg, 0.64 mmol, Intermediate IN) and AcOH (152 mg, 2.54 mmol) at rt. The resulting mixture was stirred for 1 h at 50° C. Next, to the above mixture was added NaBH₃CN (160 mg, 2.54 mmol) at rt. The resulting mixture was stirred for an additional 1 h at 50° C. On completion, the mixture was cooled to rt and purified directly by reverse phase flash chromatography (Column: WelFlash™ C18-I, 20-40 μm, 120 g; Eluent A: Water (plus 10 mM NH₄HCO₃); Eluent B: ACN; Gradient: 40%-80% B in 20 min; Flow rate: 60 mL/min; Detector: 220/254 nm; desired fractions were collected at 75% B and concentrated under reduced pressure) to afford the title compound (200 mg, 45% yield) as a yellow solid. LC/MS (ESI, m/z): [(M+1)]⁺=696.4.

Step 2—6-(4-(5-((2,8-Diazaspiro[4.5]decan-2-yl)methyl)pyridin-2-yl)indolin-1-yl)-N-((1R,2S)-2-fluorocyclopropyl)-8-(methylamino)imidazo[1,2-b]pyridazine-3-carboxamide 2,2,2-trifluoroacetate. A solution of tert-butyl 2-((6-(1-(3-(((1R,2S)-2-fluorocyclopropyl)carbamoyl)-8-(methylamino)imidazo[1,2-b]pyridazin-6-yl)indolin-4-yl)pyridin-3-yl)methyl)-2,8-diazaspiro[4.5]decane-8-carboxylate (200 mg, 0.29 mmol) in TFA (1 mL) and DCM (4 mL) was stirred for 1 h at rt. On completion, the mixture was concentrated under reduced pressure. The precipitated solids were collected by filtration and washed with diethyl ether (2×6 mL) and dried to give the title compound (190 mg, 93%) as a yellow solid. LC/MS (ESI, m/z): [(M+1)]⁺=596.6.

### 6-(4-(5-((2,7-Diazaspiro[3.5]nonan-2-yl)methyl)pyridin-2-yl)indolin-1-yl)-N-((1R,2S)-2-fluorocyclopropyl)-8-(methylamino)imidazo[1,2-b]pyridazine-3-carboxamide 2,2,2-trifluoroacetate (Intermediate PR)

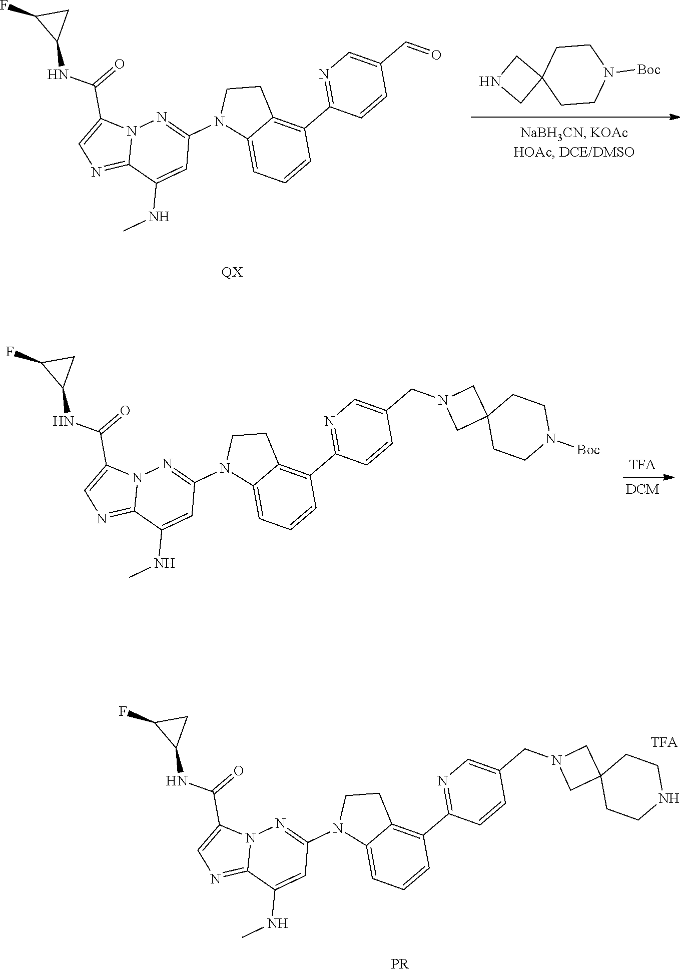

Step 1—Tert-butyl 2-({6-[1-(3-{[(1R,2S)-2-fluorocyclopropyl]carbamoyl}-8-(methylamino)imidazo[1,2-b]pyridazin-6-yl)-2,3-dihydroindol-4-yl]pyridin-3-yl}methyl)-2,7-diazaspiro[3.5]nonane-7-carboxylate. To a stirred mixture of tert-butyl 2,7-diazaspiro[3.5]nonane-7-carboxylate (2.18 g, 9.64 mmol) and KOAc (2.84 g, 28.9 mmol) in DMSO (30 mL) and DCE (30 mL) were added N-[(1R,2S)-2-fluorocyclopropyl]-6-[4-(5-formylpyridin-2-yl)-2,3-dihydroindol-1-yl]-8-(methylamino)imidazo[1,2-b]pyridazine-3-carboxamide (5 g, 10 mmol, Intermediate QX) and HOAc (2.89 g, 48.1 mmol) at rt. The resulting mixture was stirred for 40 min at 50° C. To the above mixture was added NaBH₃CN (1.21 g, 19.3 mmol) and stirred for an additional 2 h at 50° C. On completion, the mixture was cooled to rt and concentrated under vacuum. The residue was purified by reverse phase flash chromatography (Column: WelFlash™ C18-I, 20-40 μm, 330 g; Eluent A: Water (plus 5 mmol/L NH₄HCO₃); Eluent B: ACN; Gradient: 35%-65% B in 25 min; Flow rate: 100 mL/min; Detector: 220/254 nm; desired fractions were collected at 50% B and concentrated under reduced pressure) to afford the title compound (3.4 g, 52% yield) as a yellow solid. LC/MS (ESI, m/z): [(M+H)]⁺=682.4.

Step 2—6-[4-(5-{2,7-Diazaspiro[3.5]nonan-2-ylmethyl}pyridin-2-yl)-2,3-dihydroindol-1-yl]-N-[(1R,2S)-2-fluorocyclopropyl]-8-(methylamino)imidazo[1,2-b]pyridazine-3-carboxamide trifluoroacetate. To a stirred mixture of tert-butyl 2-({6-[1-(3-{[(1R,2S)-2-fluorocyclopropyl]carbamoyl}-8-(methylamino)imidazo[1,2-b]pyridazin-6-yl)-2,3-dihydroindol-4-yl]pyridin-3-yl}methyl)-2,7-diazaspiro[3.5]nonane-7-carboxylate (4.4 g, 6.5 mmol) in DCM (30 mL) was added TFA (20 mL) at rt and the mixture was stirred for 1 h at rt. On completion, the mixture was concentrated under reduced pressure. The residue was purified by trituration with diethyl ether (40 mL) to afford the title compound (3 g, 68% yield) as a yellow solid. LC/MS (ESI, m/z): [(M+H)]⁺=582.2.

### 5-(2,4-Dioxotetrahydropyrimidin-1(2H)-yl)-6-methylnicotinic acid (Intermediate PS)

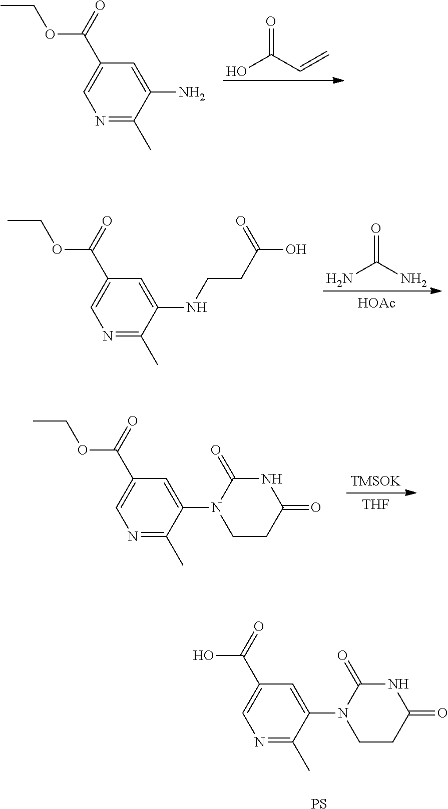

Step 1—3-((5-(Ethoxycarbonyl)-2-methylpyridin-3-yl)amino)propanoic acid. A mixture of ethyl 5-amino-6-methylnicotinate (2.00 g, 11.1 mmol, CAS #1008138-73-5) and acrylic acid (1.60 g, 22.2 mmol) was stirred for 16 h at 100° C. On completion, the mixture was cooled to rt and diluted with water (50 mL). The resulting mixture was extracted with EtOAc (3×100 mL), dried over anhydrous Na₂SO₄. After filtration, the filtrate was concentrated under reduced pressure to give the title compound (3.3 g, 94% yield) as a brown semi-solid. LC/MS (ESI, m/z): [(M+1)]=253.1

Step 2—Ethyl 5-(2,4-dioxotetrahydropyrimidin-1(2H)-yl)-6-methylnicotinate. A solution of 3-((5-(ethoxycarbonyl)-2-methylpyridin-3-yl)amino)propanoic acid (3.30 g, 10.5 mmol) and urea (4.40 g, 73.3 mmol) in AcOH (35 mL) was stirred for 16 h at 110° C. On completion, the mixture was cooled to rt and purified directly by reversed-phase flash chromatography (Column: WelFlash™ C18-I, 20-40 μm, 330 g; Eluent A: Water (plus 0.1% HCOOH); Eluent B: ACN; Gradient: 3%-25% B in 20 min; Flow rate: 80 mL/min; Detector: 204/254 nm; desired fractions were collected at 20% B and concentrated under reduced pressure) to afford the title compound (1.17 g, 40% yield) as a yellow solid. LC/MS (ESI, m/z): [(M+1)]⁺=278.2.

Step 3—5-(2,4-Dioxotetrahydropyrimidin-1(2H)-yl)-6-methylnicotinic acid. A solution of ethyl 5-(2,4-dioxotetrahydropyrimidin-1(2H)-yl)-6-methylnicotinate (100 mg, 0.36 mmol) and potassium trimethylsilanolate (231 mg, 1.81 mmol) in THF (2 mL) was stirred for 1 h at rt. On completion, the mixture was purified directly by reversed-phase flash chromatography (Column: WelFlash™ C18-I, 20-40 μm, 80 g; Eluent A: Water (plus 0.1% HCOOH); Eluent B: ACN; Gradient: 3%-20% B in 20 min; Flow rate: 40 mL/min; Detector: 204/254 nm; desired fractions were collected at 5% B and concentrated under reduced pressure) to afford the title compound (73 mg, 68% yield) as a white solid. LC/MS (ESI, m/z): [(M+1)]⁺=250.2.

### Tert-butyl 2-{[6-(2,3-dihydro-1H-indol-4-yl)pyridin-3-yl]methyl}-7-azaspiro[3.5]nonane-7-carboxylate (Intermediate PT)

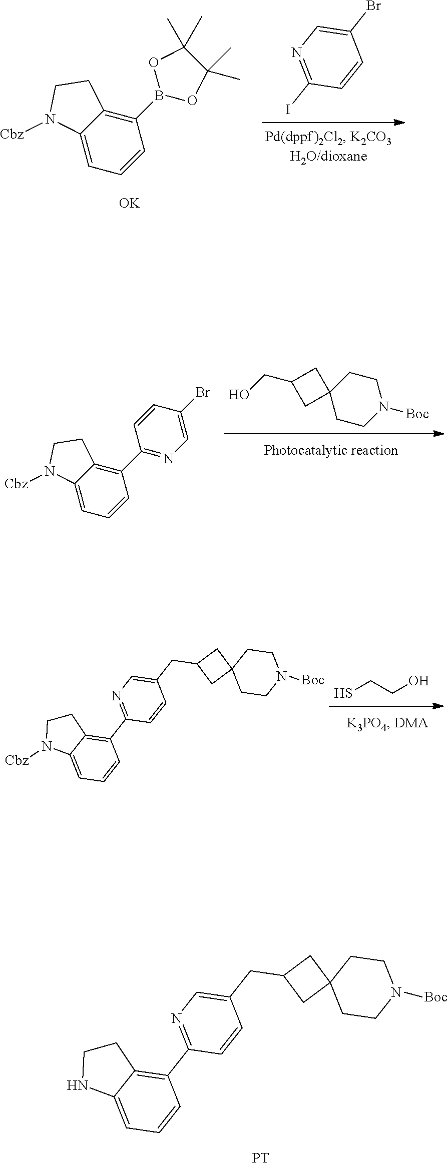

Step 1—Benzyl 4-(5-bromopyridin-2-yl)-2,3-dihydroindole-1-carboxylate. To a stirred mixture of benzyl 4-(4,4,5,5-tetramethyl-1,3,2-dioxaborolan-2-yl)-2,3-dihydroindole-1-carboxylate (3.00 g, 7.91 mmol, Intermediate OK) and 5-bromo-2-iodopyridine (2.69 g, 9.49 mmol) in dioxane (30 mL) and H₂O (3 mL) were added K₂CO₃ (3.28 g, 23.7 mmol) and Pd(dppf)Cl₂CH₂Cl₂ (0.64 g, 0.791 mmol) at rt under nitrogen atmosphere. The resulting mixture was stirred for 16 h at 80° C. under nitrogen atmosphere. On completion, the mixture was cooled to rt and concentrated under vacuum. The residue was purified by silica gel column chromatography, eluted with PE/EA (5:1), to afford the title compound (1.60 g, 49% yield) as a white solid. LC/MS (ESI, m/z): [(M+H)]⁺=409.2, 411.2.

Step 2—Tert-butyl 2-[(6-{1-[(benzyloxy)carbonyl]-2,3-dihydroindol-4-yl}pyridin-3-yl)methyl]-7-azaspiro[3.5]nonane-7-carboxylate. An oven-dried vial was charged with tert-butyl 2-(hydroxymethyl)-7-azaspiro[3.5]nonane-7-carboxylate ( ) (467.94 mg, 1.833 mmol, CAS #1356476-27-1), Deoxazole (774.54 mg, 1.955 mmol, CAS #1207294-92-5) and an X-shaped magnetic stir bar. After the vial was vacuumed and refilled with nitrogen gas twice, t-BuOMe (8 mL) was added and the reaction stirred at rt for 5 min. Then, a pyridine solution (154.61 mg, 1.955 mmol in 3 mL t-BuOMe) was added dropwise at rt within 2 min. The resulting solution stirred at room temperature for 10 min. A white solid precipitated out during this time. Another oven-dried glass flask was charged with Ir[ppy]₂(dtbbpy)PF₆ (68.65 mg, 0.061 mmol), NiBr₂(dtbbpy) (29.74 mg, 0.061 mmol), quinuclidine (237.70 mg, 2.139 mmol), 2,3-dihydro-1H-isoindole-1,3-dione (35.95 mg, 0.244 mmol), benzyl 4-(5-bromopyridin-2-yl)-2,3-dihydroindole-1-carboxylate (500 mg, 1.222 mmol) and an X-shape magnetic stir bar. DMA (10 mL) was added to this vial under an atmosphere of nitrogen. The t-BuOMe suspension was injected through the syringe filter into the DMA solution. The resulting reaction mixture was sparged with nitrogen for 15 minutes, then irradiated under 450 nm LED for 16 hr. After competition of reaction, the reaction mixture was concentrated under reduced pressure. The residue was purified by reverse phase flash chromatography (Column: WelFlash™ C18-I, 20-40 μm, 330 g; Eluent A: Water (plus 10 mmol/L FA); Eluent B: ACN; Gradient: 65%-100% B in 25 min; Flow rate: 80 mL/min; Detector: 220/254 nm; desired fractions were collected at 95% B and concentrated under reduced pressure) to afford the title compound (550 mg, 79% yield) as a brown solid. LC/MS (ESI, m/z): [(M+H)]⁺=568.3.

Step 3—Tert-butyl 2-{[6-(2,3-dihydro-1H-indol-4-yl)pyridin-3-yl]methyl}-7-azaspiro[3.5]nonane-7-carboxylate. To a stirred solution of tert-butyl 2-[(6-{1-[(benzyloxy)carbonyl]-2,3-dihydroindol-4-yl}pyridin-3-yl)methyl]-7-azaspiro[3.5]nonane-7-carboxylate (550 mg, 0.969 mmol) in DMA (10 mL) were added β-mercaptoethanol (151.38 mg, 1.938 mmol) and K₃PO₄(822.54 mg, 3.876 mmol) at rt under nitrogen atmosphere. The resulting mixture was stirred for 16 h at 70° C. under nitrogen atmosphere. On completion, the mixture was cooled to rt and purified by reverse phase flash chromatography (Column: WelFlash™ C18-I, 20-40 μm, 330 g; Eluent A: Water (plus 10 mmol/L FA); Eluent B: ACN; Gradient: 55%-85% B in 25 min; Flow rate: 80 mL/min; Detector: 220/254 nm; desired fractions were collected at 80% B and concentrated under reduced pressure) to afford the title compound (350 mg, 83% yield) as a brown oil. LC/MS (ESI, m/z): [(M+H)]-=434.2.

### 6-[4-(5-{7-Azaspiro[3.5]nonan-2-ylmethyl}pyridin-2-yl)-2,3-dihydroindol-1-yl]-N-[(1R,2S)-2-fluorocyclopropyl]-8-(methylamino)imidazo[1,2-b]pyridazine-3-carboxamide trifluoroacetate (Intermediate PU)

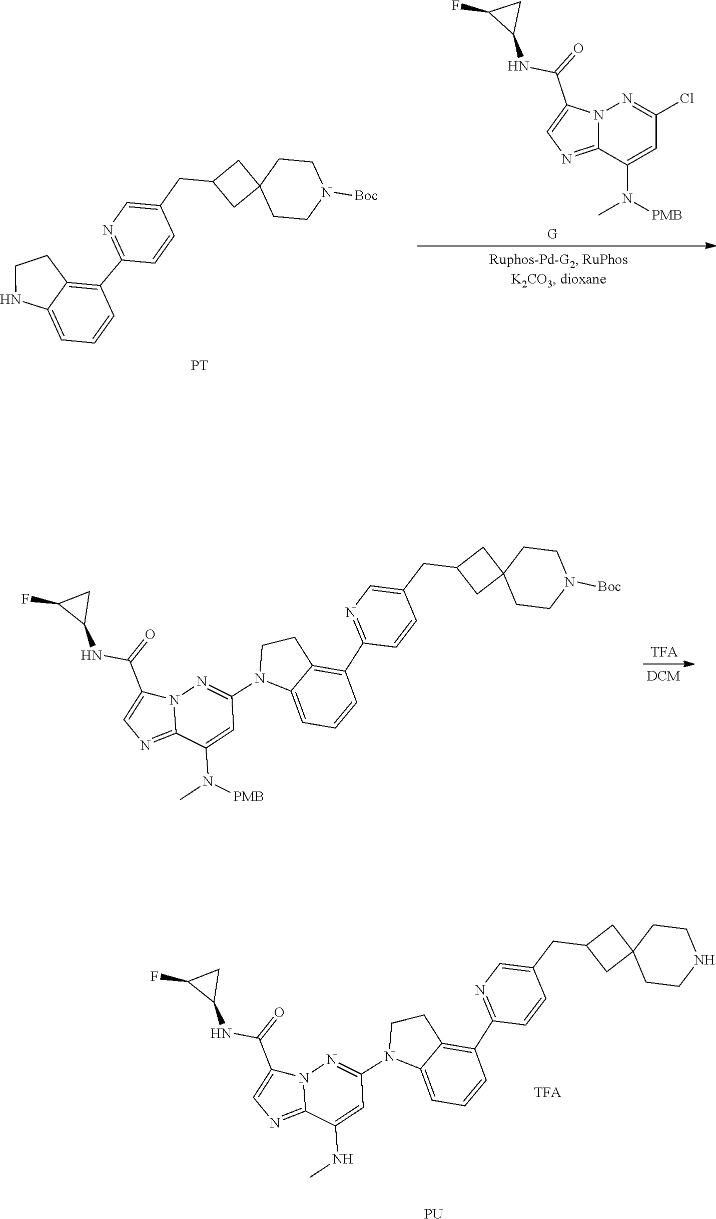

Step 1—Tert-butyl 2-({6-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyridin-3-yl}methyl)-7-azaspiro[3.5]nonane-7-carboxylate. To a stirred mixture of tert-butyl 2-{[6-(2,3-dihydro-1H-indol-4-yl)pyridin-3-yl]methyl}-7-azaspiro[3.5]nonane-7-carboxylate (350 mg, 0.807 mmol, Intermediate PT) and 6-chloro-N-[(1R,2S)-2-fluorocyclopropyl]-8-{[(4-methoxyphenyl)methyl](methyl)amino}imidazo[1,2-b]pyridazine-3-carboxamide (391.18 mg, 0.968 mmol, Intermediate G) in 1,4-dioxane (10 mL) were added K₂CO₃ (334.68 mg, 2.421 mmol), RuPhos (75.34 mg, 0.161 mmol) and RuPhos-PdCl-2nd G (125.56 mg, 0.161 mmol) at rt under nitrogen atmosphere. The resulting mixture was stirred for 2 h at 90° C. under nitrogen atmosphere. On completion, the mixture was cooled to rt and concentrated under vacuum. The residue was purified by reverse phase flash chromatography (Column: WelFlash™ C18-I, 20-40 μm, 330 g; Eluent A: Water (plus 10 mmol/L FA); Eluent B: ACN: Gradient: 70%-100% B in 25 min: Flow rate: 80 mL/min; Detector: 220/254 nm; desired fractions were collected at 97% B and concentrated under reduced pressure) to afford the title compound (530 mg, 82% yield) as a brown oil. LC/MS (ESI, m/z): [(M+H)]⁺=801.4.

Step 2—6-[4-(5-{7-Azaspiro[3.5]nonan-2-ylmethyl}pyridin-2-yl)-2,3-dihydroindol-1-yl]-N-[(1R,2S)-2-fluorocyclopropyl]-8-(methylamino)imidazo[1,2-b]pyridazine-3-carboxamide trifluoroacetate. To a stirred solution of tert-butyl 2-({6-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyridin-3-yl}methyl)-7-azaspiro[3.5]nonane-7-carboxylate (530 mg, 0.662 mmol) in DCM (4 mL) was added TFA (4 mL) at rt under nitrogen atmosphere. The resulting mixture was stirred for 1 h at rt under nitrogen atmosphere. On completion, the mixture was concentrated under vacuum. The residue was purified by trituration with Et₂O (40 mL) to afford the title compound (440 mg, 98% yield) as a yellow solid. LC/MS (ESI, m/z): [(M+H)]⁺=581.3.

### 6-{4-[5-({1,1-Difluoro-3,9-diazaspiro[5.5]undecan-3-yl}methyl)pyridin-2-yl]-2,3-dihydroindol-1-yl}-N-[(1R,2S)-2-fluorocyclopropyl]-8-(methylamino)imidazo[1,2-b]pyridazine-3-carboxamide trifluoroacetate (Intermediate PV)

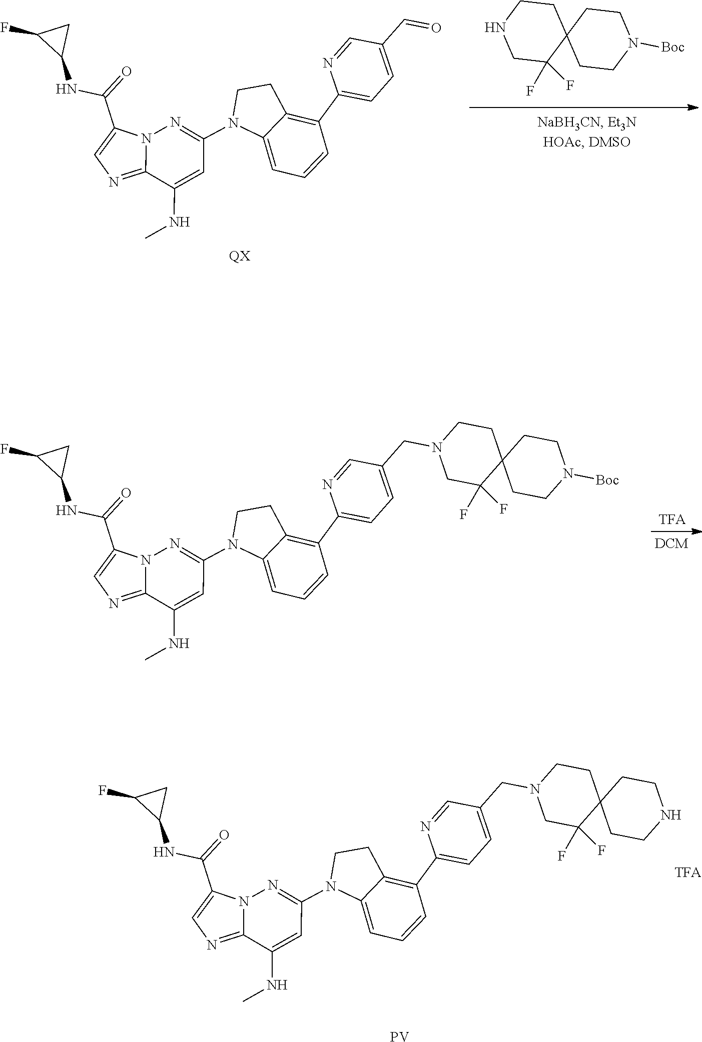

Step 1—Tert-butyl 7,7-difluoro-9-({6-[1-(3-{[(1R,2S)-2-fluorocyclopropyl]carbamoyl}-8-(methylamino)imidazo[1,2-b]pyridazin-6-yl)-2,3-dihydroindol-4-yl]pyridin-3-yl}methyl)-3,9-diazaspiro[5.5]undecane-3-carboxylate. To a stirred mixture of N-[(1R,2S)-2-fluorocyclopropyl]-6-[4-(5-formylpyridin-2-yl)-2,3-dihydroindol-1-yl]-8-(methylamino)imidazo[1,2-b]pyridazine-3-carboxamide (300 mg, 0.636 mmol, Intermediate QX) and TEA (0.1 mL, 1.9 mmol) in DMSO (3 mL) were added tert-butyl 7,7-difluoro-3,9-diazaspiro[5.5]undecane-3-carboxylate (185 mg, 0.637 mmol) and HOAc (0.2 mL, 3.5 mmol) at rt under nitrogen atmosphere. The resulting mixture was stirred for 30 min at 50° C. under nitrogen atmosphere. To the above mixture was added NaBH₃CN (199 mg, 3.167 mmol) and the mixture was stirred for an additional 2 h at 50° C. On completion, the mixture was cooled to rt and concentrated under vacuum. The residue was purified by reverse phase flash chromatography (Column: Spherical C18, 20-40 μm, 330 g; Mobile Phase A: Water (plus 10 mM NH₄HCO₃); Mobile Phase B: ACN; Flow rate: 80 mL/min; Gradient: 40%-60% B in 25 min; Detector: 254 nm; the fractions containing the desired product were collected at 55% B and concentrated under reduced pressure) to afford the title compound (100 mg, 21% yield) as light yellow oil. LC/MS (ESI, m/z): [(M+H)]⁺=746.4.

Step 2—6-(4-(5-((1,1-Difluoro-3,9-diazaspiro[5.5]undecan-3-yl)methyl)pyridin-2-yl)indolin-1-yl)-N-((1R,2S)-2-fluorocyclopropyl)-8-(methylamino)imidazo[1,2-b]pyridazine-3-carboxamide trifluoroacetate. A solution of tert-butyl 7,7-difluoro-9-({6-[1-(3-{[(1R,2S)-2-fluorocyclopropyl]carbamoyl}-8-(methylamino)imidazo[1,2-b]pyridazin-6-yl)-2,3-dihydroindol-4-yl]pyridin-3-yl}methyl)-3,9-diazaspiro[5.5]undecane-3-carboxylate (100 mg, 0.134 mmol) in DCM (1 mL) was added TFA (1 mL) at rt and the mixture was stirred for 1 h at rt under nitrogen atmosphere. On completion, the mixture was concentrated under reduced pressure. The residue was purified by trituration with diethyl ether (10 mL) to afford the title compound (80 mg, 80% yield) as a light yellow solid. LC/MS (ESI, m/z): [(M+H)]⁺=646.4.

### 3-(2,4-Dioxo-1,3-diazinan-1-yl)-4-methoxybenzoic acid (Intermediate PW)

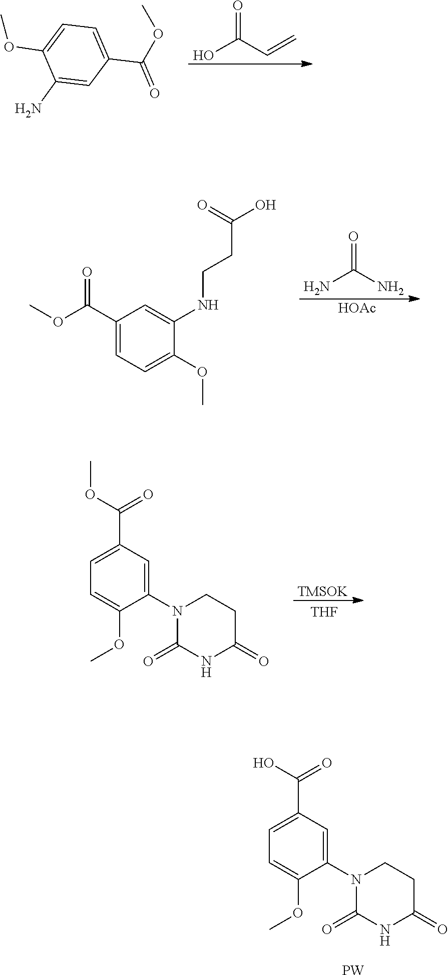

Step 1—3-((2-Methoxy-5-(methoxycarbonyl)phenyl)amino)propanoic acid. To a stirred mixture of methyl 3-amino-4-methoxybenzoate (20 g, 110 mmol) in acrylic acid (15.91 g, 220.8 mmol) at rt. The resulting mixture was stirred for 16 h at 100° C. under nitrogen atmosphere. On completion, the mixture was cooled to rt and diluted with water (200 mL). The resulting mixture was extracted with EtOAc (3×300 mL). The combined organic layers were washed with brine (1×100 mL), and dried over anhydrous Na₂SO₄. After filtration, the filtrate was concentrated under reduced pressure to afford the title compound (30 g) as a yellow solid. LC/MS (ESI, m/z): [(M+H)]^{T}=254.0.

Step 2—Methyl 3-(2,4-dioxotetrahydropyrimidin-1(2H)-yl)-4-methoxybenzoate. To a stirred mixture of 3-{[2-methoxy-5-(methoxycarbonyl)phenyl]amino}propanoic acid (28 g, 110 mmol) in HOAc (300 mL) was added urea (6.64 g, 111 mmol) at rt. The resulting mixture was stirred for 16 h at 110° C. under nitrogen atmosphere. On completion, the mixture was cooled to rt and diluted with water (300 mL) and extracted with EtOAc (3×500 mL). The combined organic layers were washed with brine (1×300 mL), and dried over anhydrous Na₂SO₄. After filtration, the filtrate was concentrated under reduced pressure. The residue was purified by trituration with tert-butyl methyl ether (500 mL) to afford the title compound (24.6 g, 80% yield) as a white solid. LC/MS (ESI, m/z): [(M+H)]-=279.0.

Step 3—3-(2,4-Dioxotetrahydropyrimidin-1(2H)-yl)-4-methoxybenzoic acid. To a stirred mixture of methyl 3-(2,4-dioxo-1,3-diazinan-1-yl)-4-methoxybenzoate (3 g, 10 mmol) in THF (100 mL) was added potassium trimethylsilanolate (6.92 g, 53.9 mmol) at rt and the mixture was stirred for 2 h at rt. On completion, the mixture was acidified to pH 3 with FA. The precipitated solids were collected by filtration and washed with water (3×10 mL). The solid was purified by trituration with water (200 mL). The resulting solid was dried by lyophilization to afford the title compound (1.8 g, 63% yield) as a white solid. LC/MS (ESI, m/z): [(M+H)]⁺=265.1.

### 1-(2-Chloro-5-{5,5-difluoro-2,7-diazaspiro[3.5]nonane-7-carbonyl}phenyl)-1,3-diazinane-2,4-dione (Intermediate PX)

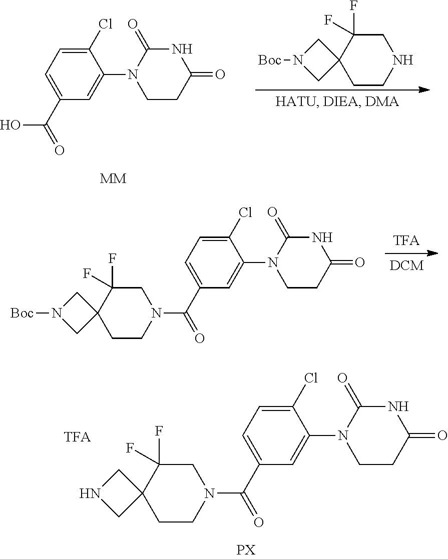

Step 1—Tert-butyl 7-[4-chloro-3-(2,4-dioxo-1,3-diazinan-1-yl)benzoyl]-5,5-difluoro-2,7-diazaspiro[3.5]nonane-2-carboxylate. To a stirred solution of 4-chloro-3-(2,4-dioxo-1,3-diazinan-1-yl)benzoic acid (1.54 g, 5.72 mmol, Intermediate MM) and HATU (3.26 g, 8.579 mmol) in DMA (30 mL) were added tert-butyl 5,5-difluoro-2,7-diazaspiro[3.5]nonane-2-carboxylate (1.5 g, 5.7 mmol) and DIEA (2.22 g, 11.2 mmol) in turns at rt under N₂ atmosphere. The resulting mixture was stirred for 1 h at it under N₂ atmosphere. On completion, the mixture was purified directly by reversed-phase flash chromatography (column, C18 silica gel; mobile phase, MeCN in Water (10 mmol/L NH₄HCO₃), 50% to 95% gradient in 30 min; detector, UV 254 nm; the fractions containing the desired product were collected at 75% B and concentrated under reduced pressure) to afford the title compound (2 g, 68% yield) as a light yellow solid. LC/MS (ESI, m/z): [(M+H)]⁺=513.2.

Step 2—1-(2-Chloro-5-{5,5-difluoro-2,7-diazaspiro[3.5]nonane-7-carbonyl}phenyl)-1,3-diazinane-2,4-dione trifluoroacetate. To a stirred solution of tert-butyl 7-[4-chloro-3-(2,4-dioxo-1,3-diazinan-1-yl)benzoyl]-5,5-difluoro-2,7-diazaspiro[3.5]nonane-2-carboxylate (2 g, 4 mmol) in DCM (30 mL) was added TFA (10 mL) at rt and the mixture was stirred for 1 h at rt. After the completion of reaction, the mixture was concentrated under reduced pressure. The residue was purified by trituration with ethyl ether (50 mL) to afford the title compound (1.5 g, 93% yield) as a light brown. LC/MS (ESI, m/z): [(M+H)]⁺=413.2.

### 4-(5-(1,3-Dioxolan-2-yl)pyridin-2-yl)-6-methoxyindoline (Intermediate PY)

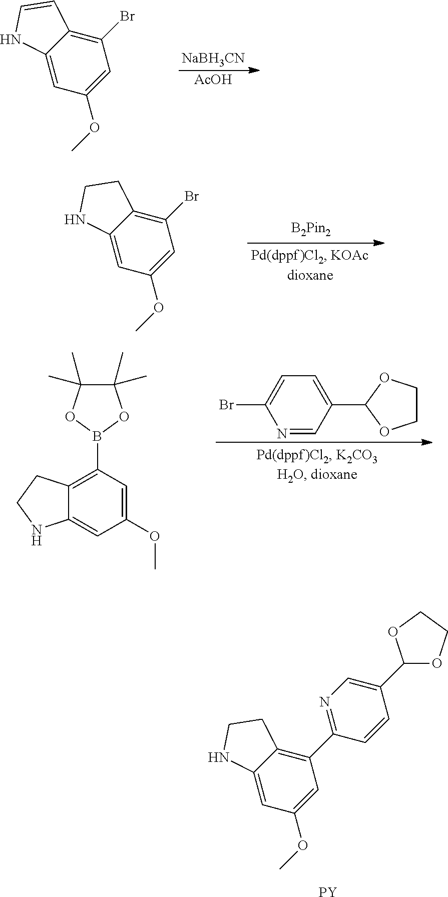

Step 1—4-Bromo-6-methoxyindoline. To a stirred mixture of 4-bromo-6-methoxy-1H-indole (3 g, 10 mmol, CAS #393553-55-4) in HOAc (30 mL) was added NaBH₃CN (4.17 g, 66.4 mmol) at 0° C. The resulting mixture was stirred for 1 h at rt. On completion, the mixture was basified to pH 8 with saturated NaOH (aq.) and extracted with EtOAc (3×200 mL). The combined organic layers were washed with brine (1×100 mL), dried over anhydrous Na₂SO, filtered, and the filtrate was concentrated under reduced pressure. The residue was purified by silica gel column chromatography, eluted with CH₂Cl₂/EA (3:1), to afford the title compound (2 g, 60% yield) as a yellow oil. LC/MS (ESI, m/z): [(M+H)]⁺=228.1, 230.1.

Step 2—6-Methoxy-4-(4,4,5,5-tetramethyl-1,3,2-dioxaborolan-2-yl)indoline. To a stirred mixture of 4-bromo-6-methoxy-2,3-dihydro-1H-indole (1.9 g, 8.3 mmol) and bis(pinacolato)diboron (2.12 g, 8.33 mmol) in dioxane (20 mL) were added KOAc (2.45 g, 25.0 mmol) and Pd(dppf)Cl₂CH₂Cl₂ (1.36 g, 1.67 mmol) at rt under nitrogen atmosphere. The resulting mixture was stirred for 2 h at 90° C. under nitrogen atmosphere. On completion, the mixture was cooled to rt and concentrated under vacuum. The residue was purified by silica gel column chromatography (Mobile Phase A: PE, Mobile Phase B: EA; Gradient: 0% B to 30% B in 25 min, 254 nm; the fractions containing the desired product were collected at 25% B) to afford the title compound (1.6 g, 70% yield) as a yellow oil. LC/MS (ESI, m/z): [(M+H)]⁺=276.2.

Step 3—4-(5-(1,3-Dioxolan-2-yl)pyridin-2-yl)-6-methoxyinoline. To a stirred mixture of 6-methoxy-4-(4,4,5,5-tetramethyl-1,3,2-dioxaborolan-2-yl)indoline (1.6 g, 5.815 mmol) and 2-bromo-5-(1,3-dioxolan-2-yl)pyridine (1.34 g, 5.82 mmol, CAS #220904-17-6) in dioxane (15 mL) and H₂O (3 mL) were added K₂CO₃ (2.41 g, 17.5 mmol) and Pd(dppf)Cl₂CH₂Cl₂ (0.95 g, 1.16 mmol) at rt under nitrogen atmosphere. The resulting mixture was stirred for 16 h at 90° C. under nitrogen atmosphere. On completion, the mixture was cooled to rt and concentrated under vacuum. The residue was purified by reverse phase flash chromatography (Column: Spherical C18, 20-40 μm, 330 g; Mobile Phase A: Water (plus 10 mM NH₄HCO₃); Mobile Phase B: ACN; Flow rate: 80 mL/min; Gradient: 50% B-70% B in 20 min; Detector: 254 nm; the fractions containing desired product were collected at 60% B and concentrated under reduced pressure) to afford the title compound (660 mg, 38% yield) as a yellow oil. LC/MS (ESI, m/z): [(M+H)]+=299.1.

### N-((1R,2S)-2-fluorocyclopropyl)-6-(4-(5-formylpyridin-2-yl)-6-methoxyindolin-1-yl)-8-(methylamino)imidazo[1,2-b]pyridazine-3-carboxamide (Intermediate PZ)

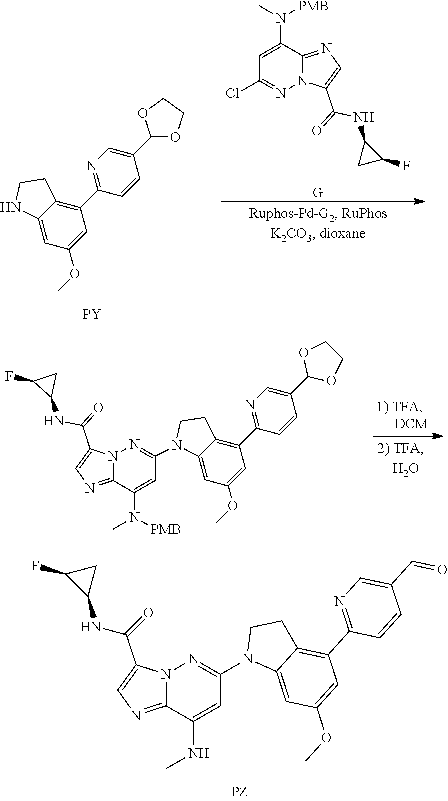

Step 1—6-(4-(5-(1,3-Dioxolan-2-yl)pyridin-2-yl)-6-methoxyindolin-1-yl)-N-((1R,2S)-2-fluorocyclopropyl)-8-((4-methoxybenzyl)(methyl)amino)imidazo[1,2-b]pyridazine-3-carboxamide. To a stirred mixture of 4-(5-(1,3-dioxolan-2-yl)pyridin-2-yl)-6-methoxyindoline (300 mg, 1.01 mmol, Intermediate PY) and 6-chloro-N-[(1R,2S)-2-fluorocyclopropyl]-8-{[(4-methoxyphenyl)methyl](methyl)amino}imidazo[1,2-b]pyridazine-3-carboxamide (406.08 mg, 1.006 mmol, Intermediate G) in dioxane (8 mL) were added K₂CO₃ (416.92 mg, 3.018 mmol) and RuPhos (46.92 mg, 0.101 mmol) at rt under nitrogen atmosphere. To the above mixture was added RuPhos-PdCl-2nd G (78.21 mg, 0.101 mmol) at rt under nitrogen atmosphere. The resulting mixture was stirred for an additional 2 h at 90° C. On completion, the mixture was cooled to rt and concentrated under vacuum. The residue was purified by reverse phase flash chromatography (Column: Spherical C18, 20-40 μm, 330 g; Mobile Phase A: Water (plus 10 mM NH₄HCO₃); Mobile Phase B: ACN; Flow rate: 80 mL/min; Gradient: 65% B-85% B in 20 min; Detector: 254 nm; the fractions containing desired product were collected at 75% B and concentrated under reduced pressure) to give the title compound (300 mg, 45% yield) as a yellow oil. LC/MS (ESI, m/z): [(M+H)]⁺=666.2.

Step 2—N-((1R,2S)-2-fluorocyclopropyl)-6-(4-(5-formylpyridin-2-yl)-6-methoxyindolin-1-yl)-8-(methylamino)imidazo[1,2-b]pyridazine-3-carboxamide. To a stirred solution of 6-{4-[5-(1,3-dioxolan-2-yl)pyridin-2-yl]-6-methoxy-2,3-dihydroindol-1-yl}-N-[(R,2S)-2-fluorocyclopropyl]-8-{1[(4-methoxyphenyl)methyl](methyl)amino}imidazo[1,2-b]pyridazine-3-carboxamide (300 mg, 0.451 mmol) in DCM (3 mL) was added TFA (2 mL) at rt under nitrogen atmosphere. The resulting mixture was stirred for 1 h at rt. Then, the mixture was concentrated under vacuum. To the above mixture was added H₂O (5 mL) and TFA (1 mL) at rt. The resulting mixture was stirred for 2 h at 50° C. On completion, the mixture was cooled to rt and basified to pH 8 with saturated NaOH (aq.). The resulting mixture was extracted with EtOAc (3×50 mL). The combined organic layers were washed with brine (1×50 mL), dried over anhydrous Na₂SO₄, filtered, and the filtrate was concentrated under reduced pressure to give the title compound (290 mg) as a yellow solid. LC/MS (ESI, m/z): [(M+H)]⁺=502.1.

### 4-[5-(1,3-Dioxolan-2-yl)pyridin-2-yl]-6-methyl-2,3-dihydro-1H-indole (Intermediate QA)

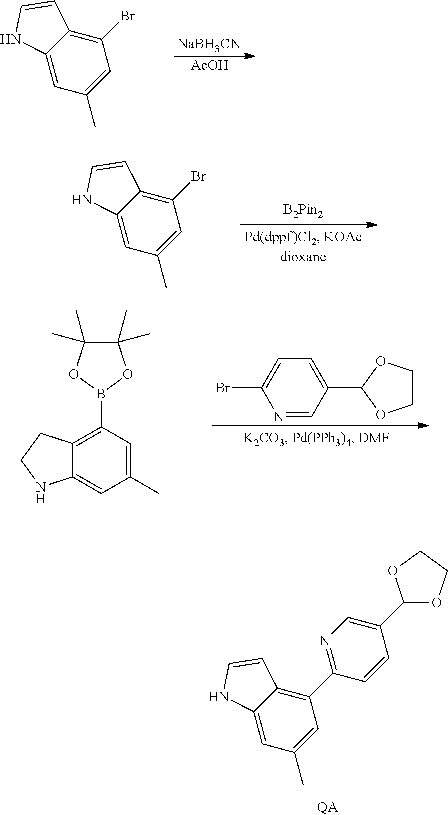

Step 1—4-Bromo-6-methyl-2,3-dihydro-1H-indole. To a stirred mixture of 4-bromo-6-methyl-1H-indole (2 g, 10 mmol, CAS #885520-48-9) in HOAc (20 mL) was added NaBH₃CN (2.99 g, 47.6 mmol) at rt under nitrogen atmosphere and the mixture was stirred for 1 h at rt. On completion, the mixture was concentrated under reduced pressure. The reaction was quenched by water (50 mL) at rt and the mixture was extracted with EtOAc (3×50 mL). The combined organic layers were washed with brine (1×50 mL), dried over anhydrous Na₂SO₄, filtered, and the filtrate was concentrated under reduced pressure. The residue was purified by silica gel column chromatography, eluted with PE/EA (5:1), to afford the title compound (1.6 g, 79% yield) as a colorless liquid. LC/MS (ESI, m/z): [(M+H)]⁺=212.0, 214.0.

Step 2—6-Methyl-4-(4,4,5,5-tetramethyl-1,3,2-dioxaborolan-2-yl)-2,3-dihydro-1H-indole. To a stirred mixture of 4-bromo-6-methyl-2,3-dihydro-1H-indole (1.6 g, 7.5 mmol) and 4,4,5,5-tetramethyl-2-(4,4,5,5-tetramethyl-1,3,2-dioxaborolan-2-yl)-1,3,2-dioxaborolane (3.83 g, 15.1 mmol) in dioxane (16 mL) were added KOAc (2.22 g, 22.6 mmol) and Pd(dppf)Cl₂CH₂Cl₂ (0.61 g, 0.75 mmol) at rt under nitrogen atmosphere. The resulting mixture was stirred for an additional 2 h at 90° C. On completion, the mixture was cooled to rt and concentrated under reduced pressure. The residue was purified by silica gel column chromatography, eluted with PE/EA (3:1), to afford the title compound (1.5 g, 77% yield) as a yellow solid. LC/MS (ESI, m/z): [(M+H)]=260.1.

Step 3—4-[5-(1,3-Dioxolan-2-yl)pyridin-2-yl]-6-methyl-2,3-dihydro-1H-indole. To a stirred mixture of 6-methyl-4-(4,4,5,5-tetramethyl-1,3,2-dioxaborolan-2-yl)-2,3-dihydro-1H-indole (1.5 g, 5.8 mmol) and 2-bromo-5-(1,3-dioxolan-2-yl)pyridine (1.11 g, 4.82 mmol) in DMF (15 mL) were added K₂CO₃ (2.00 g, 14.5 mmol) and Pd(PPh₃)₄(0.56 g, 0.482 mmol) at rt under nitrogen atmosphere. The resulting mixture was stirred for an additional 16 h at 90° C. On completion, the mixture was cooled to it and purified by reverse phase flash chromatography (Column: WelFlash™ C18-I, 20-40 μm, 330 g; Eluent A: Water (plus 10 mmol/L FA); Eluent B: ACN; Gradient: 15%-35% B in 25 min; Flow rate: 80 mL/min; Detector: 220/254 nm; desired fractions were collected at 19% B and concentrated under reduced pressure) to afford the title compound (150 mg, 11% yield) as a brown oil. LC/MS (ESI, m/z): [(M+H)]f=283.1.

### N-[(1R,2S)-2-fluorocyclopropyl]-6-[4-(5-formylpyridin-2-yl)-6-methyl-2,3-dihydroindol-1-yl]-8-(methylamino)imidazo[1,2-b]pyridazine-3-carboxamide (Intermediate QB)

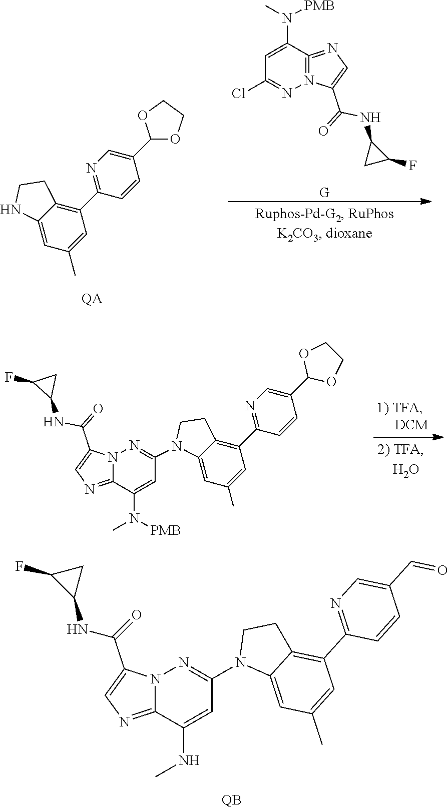

Step 1—6-{4-[5-(1,3-Dioxolan-2-yl)pyridin-2-yl]-6-methyl-2,3-dihydroindol-1-yl}-N-[(1R,2S)-2-fluorocyclopropyl]-8-{[(4-methoxyphenyl)methyl](methyl)amino}imidazo[1,2-b]pyridazine-3-carboxamide. To a stirred mixture of 4-[5-(1,3-dioxolan-2-yl)pyridin-2-yl]-6-methyl-2,3-dihydro-1H-indole (150 mg, 0.531 mmol, Intermediate QA) and 6-chloro-N-[(1R,2S)-2-fluorocyclopropyl]-8-{[(4-methoxyphenyl)methyl](methyl)amino}imidazo[1,2-b]pyridazine-3-carboxamide (214.55 mg, 0.531 mmol, Intermediate G) in dioxane (5 mL) were added K₂CO₃ (220.27 mg, 1.593 mmol), RuPhos (24.79 mg, 0.053 mmol) and RuPhos-PdCl-2nd G (41.32 mg, 0.053 mmol) at rt under nitrogen atmosphere. The resulting mixture was stirred for additional 2 h at 90° C. On completion, the mixture was cooled to rt and concentrated under reduced pressure. The residue was purified by silica gel column chromatography, eluted with CH₂Cl₂/EA (1:1), to afford the title compound (200 mg, 58% yield) as a yellow solid. LC/MS (ESI, m/z): [(M+H)]⁺=650.5.

Step 2—N-[(1R,2S)-2-fluorocyclopropyl]-6-[4-(5-formylpyridin-2-yl)-6-methyl-2,3-dihydroindol-1-yl]-8-(methylamino)imidazo[1,2-b]pyridazine-3-carboxamide. To a stirred mixture of 6-{4-[5-(1,3-dioxolan-2-yl)pyridin-2-yl]-6-methyl-2,3-dihydroindol-1-yl}-N-[(1R,2S)-2-fluorocyclopropyl]-8-{[(4-methoxyphenyl)methyl](methyl)amino}imidazo[1,2-b]pyridazine-3-carboxamide (200 mg, 0.308 mmol) in DCM (2 mL) was added TFA (1 mL) at rt under nitrogen atmosphere and the mixture was stirred for 1 h at rt. Then mixture was concentrated under reduced pressure. This resulted in 6-{4-[5-(1,3-dioxolan-2-yl)pyridin-2-yl]-6-methyl-2,3-dihydroindol-1-yl}-N-[(1R,2S)-2-fluorocyclopropyl]-8-(methylamino)imidazo[1,2-b]pyridazine-3-carboxamide. To a stirred mixture of 6-{4-[5-(1,3-dioxolan-2-yl)pyridin-2-yl]-6-methyl-2,3-dihydroindol-1-yl}-N-[(1R,2S)-2-fluorocyclopropyl]-8-(methylamino)imidazo[1,2-b]pyridazine-3-carboxamide in TFA (5 mL) was added H₂O (1 mL) at rt under nitrogen atmosphere. The resulting mixture was stirred for additional 2 h at 50° C. On completion, the mixture was cooled to rt and basified to pH 9 with NaOH (aq.). The resulting mixture was extracted with EtOAc (3×20 mL). The combined organic layers were dried over anhydrous Na₂SO₄, filtered, and the filtrate was concentrated under reduced pressure to give the title compound (180 mg, 98% yield) as a yellow solid. LC/MS (ESI, m/z): [(M+H)]⁺=486.4.

### Benzyl 4-[(3,3-difluoropiperidin-4-yl)methyl]-2,3-dihydroindole-1-carboxylate trifluoroacetate (Intermediate QC)

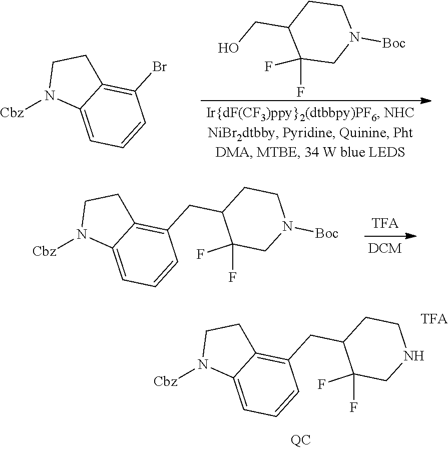 [SMILES: O=C(OCc1ccccc1)N1CCc2c(Br)cccc21 ; CC(C)(C)OC(=O)N1CCC(CO)C(F)(F)C1 ; CC(C)(C)OC(=O)N1CCC(Cc2cccc3c2CCN3C(=O)OCc2ccccc2)C(F)(F)C1 ; O=C(OCc1ccccc1)N1CCc2c(CC3CCNCC3(F)F)cccc21]

Step 1—Benzyl 4-{[1-(tert-butoxycarbonyl)-3,3-difluoropiperidin-4-yl]methyl}-2,3-dihydroindole-1-carboxylate. An oven-dried 100 mL vial was charged with tert-butyl 3,3-difluoro-4-(hydroxymethyl)piperidine-1-carboxylate (1.13 g, 4.52 mmol, CAS #1303974-47-1), Deoxazole (1.91 g, 4.82 mmol, CAS #1207294-92-5) and an X-shaped magnetic stir bar. After the vial was vacuumed and refilled with nitrogen gas twice, t-BuOMe (15 mL) was added and the reaction stirred at rt for 5 min. Then, a pyridine solution (0.38 g, 4.82 mmol in 5 mL t-BuOMe) was added dropwise at rt within 2 min. The resulting solution stirred at rt for 10 min. A white solid precipitated out during this time. Another oven-dried glass flask was charged with Ir[ppy]₂(dtbbpy)PF₆ (0.17 g, 0.150 mmol), NiBr₂(dtbbpy) (0.07 g, 0.150 mmol), quinuclidine (0.59 g, 5.27 mmol), 2,3-dihydro-1H-isoindole-1,3-dione (0.09 g, 0.602 mmol), benzyl 4-bromo-2,3-dihydroindole-1-carboxylate (1.0 g, 3.01 mmol, synthesized via Step 1 of Intermediate OK) and an X-shape magnetic stir bar. DMA (20 mL) was added to this vial under an atmosphere of nitrogen. The t-BuOMe suspension was injected through the syringe filter into the DMA solution. The resulting reaction mixture was sparged with nitrogen for 15 minutes, then irradiated under 450 nm LED for 16 hrs. After competition of reaction, the reaction mixture was concentrated under reduced pressure and the mixture was diluted with water (150 mL) and extracted with EtOAc (3×100 mL). The combined organic layers were washed with brine (1×100 mL), dried over anhydrous Na₂SO₄, filtered, and the filtrate was concentrated under reduced pressure. The residue was purified by silica gel column chromatography, eluted with PE/EA (5:1), to afford the title compound (1.0 g, 68% yield) as a light yellow solid. LC/MS (ESI, m/z): [(M+H)]⁺=487.2.

Step 2—Benzyl 4-[(3,3-difluoropiperidin-4-yl)methyl]-2,3-dihydroindole-1-carboxylate trifluoroacetate. To a stirred solution of benzyl 4-{[1-(tert-butoxycarbonyl)-3,3-difluoropiperidin-4-yl]methyl}-2,3-dihydroindole-1-carboxylate (1.0 g, 2.1 mmol) in DCM (6 mL) was added TFA (3 mL) at rt under nitrogen atmosphere and the mixture was stirred for 1 h at rt under nitrogen atmosphere. On completion, the mixture was concentrated under vacuum. The residue was purified by trituration with Et₂O (50 mL) to afford the title compound (900 mg, 90% yield) as a light yellow solid. LC/MS (ESI, m/z): [(M+H)]⁺=387.1.

### Tert-butyl 9-[4-(2,3-dihydro-1H-indol-4-ylmethyl)-3,3-difluoropiperidin-1-yl]-3-azaspiro[5.5]undecane-3-carboxylate (Intermediate QD)

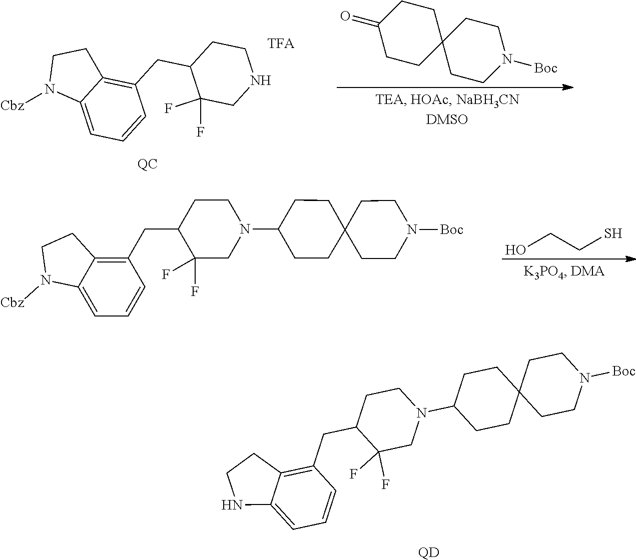

Step 1—Tert-butyl 9-[4-({1-[(benzyloxy)carbonyl]-2,3-dihydroindol-4-yl}methyl)-3,3-difluoropiperidin-1-yl]-3-azaspiro[5.5]undecane-3-carboxylate. To a stirred solution of benzyl 4-[(3,3-difluoropiperidin-4-yl)methyl]-2,3-dihydroindole-1-carboxylate trifluoroacetate (900 mg, 1.86 mmol, Intermediate QC) in DMSO (10 mL) was added TEA (0.77 mL, 5.57 mmol) at rt under nitrogen atmosphere. To the above mixture were added tert-butyl 9-oxo-3-azaspiro[5.5]undecane-3-carboxylate (496.69 mg, 1.858 mmol, CAS #873924-08-4) and AcOH (0.32 mL, 5.57 mmol) at rt. The resulting mixture was stirred for an additional 1 h at 50° C. The mixture was allowed to cool to rt then NaBH₃CN (233.48 mg, 3.716 mmol) was added at rt. The resulting mixture was stirred for additional 16 h at 50° C. On completion, the mixture was cooled to rt and concentrated under vacuum. The residue was purified by reverse phase flash chromatography (Column: WelFlash™ C18-I, 20-40 μm, 330 g; Eluent A: Water (plus 10 mmol/L FA); Eluent B: ACN; Gradient: 55%-100% B in 30 min; Flow rate: 80 mL/min; Detector: 220/254 nm; desired fractions were collected at 100% B and concentrated under reduced pressure) to afford the title compound (1.0 g, 84% yield) as a brown oil. LC/MS (ESI, m/z): [(M+H)]⁺=638.3.

Step 2—Tert-butyl 9-[4-(2,3-dihydro-1H-indol-4-ylmethyl)-3,3-difluoropiperidin-1-yl]-3-azaspiro[5.5]undecane-3-carboxylate. To a stirred mixture of tert-butyl 9-[4-({1-[(benzyloxy)carbonyl]-2,3-dihydroindol-4-yl}methyl)-3,3-difluoropiperidin-1-yl]-3-azaspiro[5.5]undecane-3-carboxylate (1.0 g, 1.568 mmol) and #-mercaptoethanol (0.24 g, 3.14 mmol) in DMA (10 mL) was added K₃PO₄ (1.33 g, 6.27 mmol) at rt under nitrogen atmosphere. The resulting mixture was stirred for 16 h at 75° C. under nitrogen atmosphere. On completion, the mixture was cooled to rt and purified by reverse phase flash chromatography (Column: WelFlash™ C18-I, 20-40 μm, 330 g; Eluent A: Water (plus 10 mmol/L NH₄HCO₃); Eluent B: ACN; Gradient: 65%-100% B in 25 min; Flow rate: 80 mL/min; Detector: 220/254 nm; desired fractions were collected at 95% B and concentrated under reduced pressure) to afford the title compound (650 mg, 82% yield) as an off-white solid. LC/MS (ESI, m/z): [(M+H)]⁺=504.3.

### 6-{4-[(1-{3-Azaspiro[5.5]undecan-9-yl}-3,3-difluoropiperidin-4-yl)methyl]-2,3-dihydroindol-1-yl}-N-[(1R,2S)-2-fluorocyclopropyl]-8-(methylamino)imidazo[1,2-b]pyridazine-3-carboxamide trifluoroacetate (Intermediate QE)

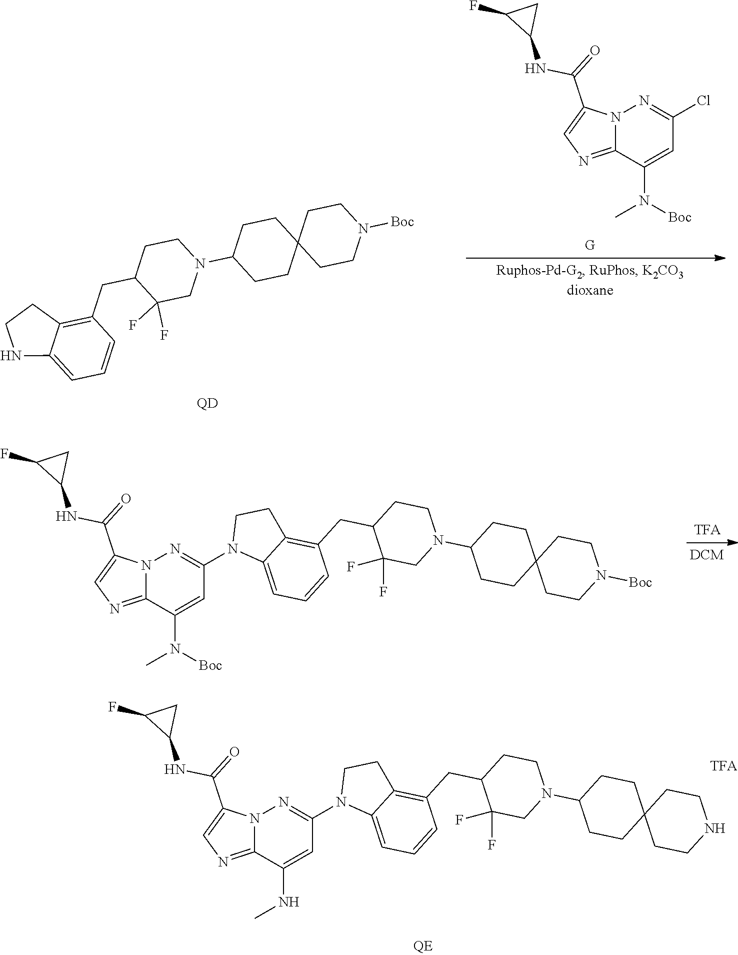

Step 1—Tert-butyl 9-{4-[(1-{8-[(tert-butoxycarbonyl)(methyl)amino]-3-{[(1R,2S)-2-fluorocyclopropyl]carbamoyl}imidazo[1,2-b]pyridazin-6-yl}-2,3-dihydroindol-4-yl)methyl]-3,3-difluoropiperidin-1-yl}-3-azaspiro[5.5]undecane-3-carboxylate. To a stirred mixture of tert-butyl 9-[4-(2,3-dihydro-1H-indol-4-ylmethyl)-3,3-difluoropiperidin-1-yl]-3-azaspiro[5.5]undecane-3-carboxylate (300 mg, 0.596 mmol, Intermediate QD) and tert-butyl N-(6-chloro-3-{[(1R,2S)-2-fluorocyclopropyl]carbamoyl}imidazo[1,2-b]pyridazin-8-yl)-N-methylcarbamate (228.60 mg, 0.596 mmol, Intermediate G) in 1,4-dioxane (10 mL) were added K₂CO₃ (246.95 mg, 1.788 mmol), RuPhos (55.59 mg, 0.119 mmol) and RuPhos-PdCl-2nd G (92.65 mg, 0.119 mmol) at rt under nitrogen atmosphere. The resulting mixture was stirred for 1 h at 90° C. under nitrogen atmosphere. On completion, the mixture was cooled to rt and concentrated under vacuum. The residue was purified by reverse phase flash chromatography (Column: WelFlash™ C18-I, 20-40 μm, 330 g; Eluent A: Water (plus 10 mmol/L FA); Eluent B: ACN; Gradient: 65%-100% B in 25 min; Flow rate: 80 mL/min; Detector: 220/254 nm; desired fractions were collected at 95% B and concentrated under reduced pressure) to afford the title compound (450 mg, 89% yield) as a brown solid. LC/MS (ESI, m/z): [(M+H)]⁺=851.0.

Step 2—6-{4-[(1-{3-azaspiro[5.5]undecan-9-yl}-3,3-difluoropiperidin-4-yl)methyl]-2,3-dihydroindol-1-yl}-N-[(1R,2S)-2-fluorocyclopropyl]-8-(methylamino)imidazo[1,2-b]pyridazine-3-carboxamide trifluoroacetate. To a stirred solution of tert-butyl 9-{4-[(1-{8-[(tert-butoxycarbonyl)(methyl)amino]-3-{[(1R,2S)-2-fluorocyclopropyl]carbamoyl}imidazo[1,2-b]pyridazin-6-yl}-2,3-dihydroindol-4-yl)methyl]-3,3-difluoropiperidin-1-yl}-3-azaspiro[5.5]undecane-3-carboxylate (450 mg, 0.529 mmol) in DCM (6 mL) was added TFA (3 mL) at rt under nitrogen atmosphere and the mixture was stirred for 1 h at rt under nitrogen atmosphere. On completion, the mixture was concentrated under vacuum. The residue was purified by trituration with Et₂O (50 mL) to afford the title compound (390 mg, 99% yield) as a brown solid. LC/MS (ESI, m/z): [(M+H)]⁺=651.3.

### 6-{4-[(4-{3,9-Diazaspiro[5.5]undecan-3-yl}-3,3-difluoropiperidin-1-yl)methyl]-2,3-dihydroindol-1-yl}-N-[(1R,2S)-2-fluorocyclopropyl]-8-(methylamino)imidazo[1,2-b]pyridazine-3-carboxamide trifluoroacetate (Intermediate QF)

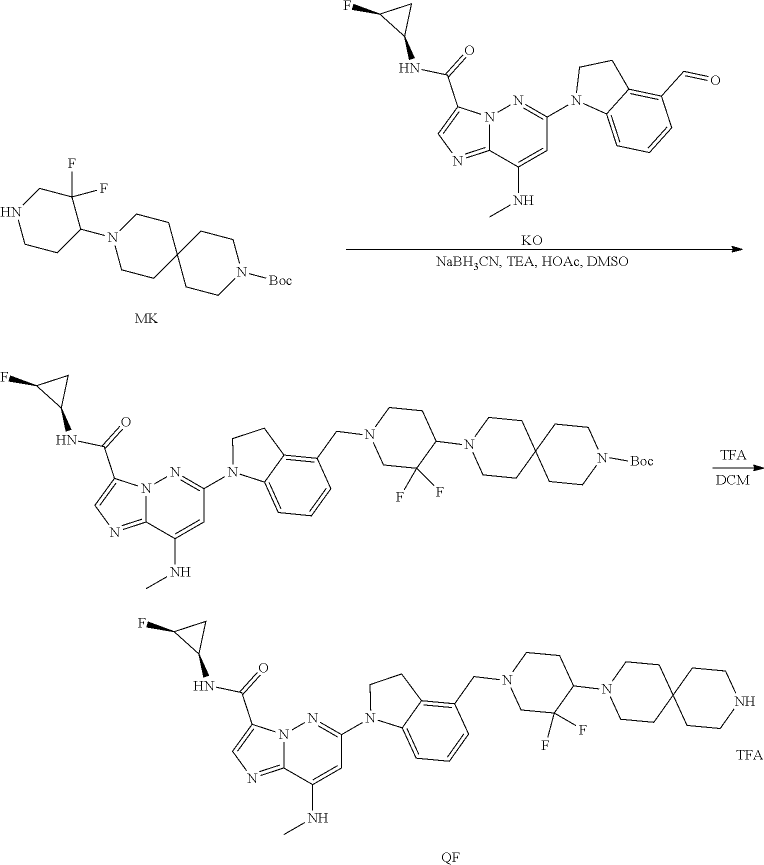

Step 1—Tert-butyl 9-(3,3-difluoro-1-{[1-(3-{[(1R,2S)-2-fluorocyclopropyl]carbamoyl}-8-(methylamino)imidazo[1,2-b]pyridazin-6-yl)-2,3-dihydroindol-4-yl]methyl}piperidin-4-yl)-3,9-diazaspiro[5.5]undecane-3-carboxylate. To a stirred mixture of tert-butyl 9-(3,3-difluoropiperidin-4-yl)-3,9-diazaspiro[5.5]undecane-3-carboxylate (200 mg, 0.535 mmol, Intermediate MK) and N-[(1R,2S)-2-fluorocyclopropyl]-6-(4-formyl-2,3-dihydroindol-1-yl)-8-(methylamino)imidazo[1,2-b]pyridazine-3-carboxamide (211.20 mg, 0.535 mmol, Intermediate KO) in DMSO (4 mL) was added Et₃N (108.38 mg, 1.070 mmol) dropwise rt. To the above mixture was added HOAc (321.57 mg, 5.350 mmol) dropwise at 50° C. The resulting mixture was stirred for an additional 1 h at rt. To the above mixture was added NaBH₃CN (168.25 mg, 2.675 mmol) in portions at rt. The resulting mixture was stirred for an additional 1 h at 50° C. On completion, the mixture was cooled to rt and purified directly by reversed-phase flash chromatography (Column: Spherical C18, 20-40 μm, 330 g; Mobile Phase A: Water (plus 10 mM NH₄HCO₃), Mobile Phase B: ACN; Flow rate: 80 mL/min; Gradient (B %): 5%80%, 4 min; 80%˜95%, 20 min; 95%-95%; 2 min; 95%, 5 min; Detector: 254 nm; the fractions containing desired product were collected at 94% B and concentrated under reduced pressure) to afford the title compound (260 mg, 65% yield) as a white solid. LC/MS (ESI, m/z): [(M+H)]⁺=752.4.

Step 2—6-{4-[(4-{3,9-Diazaspiro[5.5]undecan-3-yl}-3,3-difluoropiperidin-1-yl)methyl]-2,3-dihydroindol-1-yl}-N-[(1R,2S)-2-fluorocyclopropyl]-8-(methylamino)imidazo[1,2-b]pyridazine-3-carboxamide trifluoroacetate. To a stirred solution of tert-butyl 9-(3,3-difluoro-1-{[1-(3-{[(1R,2S)-2-fluorocyclopropyl]carbamoyl}-8-(methylamino)imidazo[1,2-b]pyridazin-6-yl)-2,3-dihydroindol-4-yl]methyl}piperidin-4-yl)-3,9-diazaspiro[5.5]undecane-3-carboxylate (80 mg, 0.11 mmol) in DCM (3 mL) was added TEA (1 mL) dropwise at rt and the mixture was stirred for 1 h at rt. On completion, the mixture was concentrated under reduced pressure. The mixture product was purified by trituration with Et₂O (10 mL) to afford the title compound (75 mg, 94% yield) as a brown solid. LC/MS (ESI, m/z): [(M+H)]+=652.4.

### N-cyclopropyl-6-(4-formyl-2,3-dihydroindol-1-yl)-8-(methylamino)imidazo[1,2-b]pyridazine-3-carboxamide (Intermediate QG)

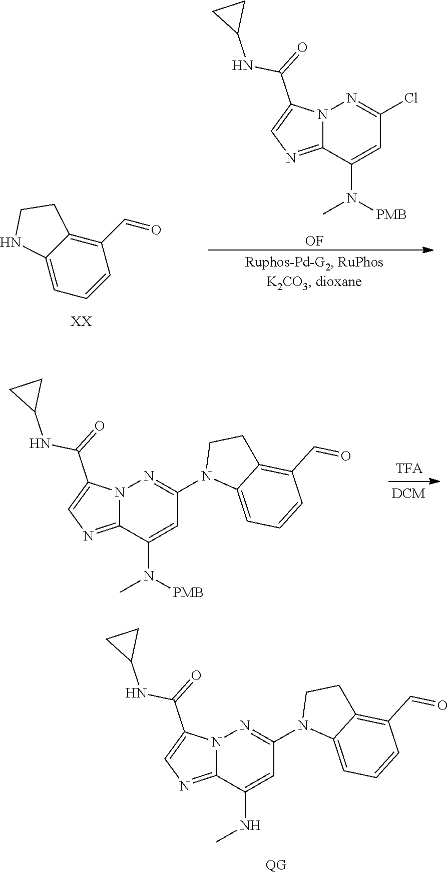

Step 1—N-cyclopropyl-6-(4-formyl-2,3-dihydroindol-1-yl)-8-{[(4-methoxyphenyl)methyl](methyl)amino}imidazo[1,2-b]pyridazine-3-carboxamide. To a stirred mixture of indoline-4-carbaldehyde (1.2 g, 8.2 mmol, Intermediate XX) and 6-chloro-N-cyclopropyl-8-{[(4-methoxyphenyl)methyl](methyl)amino}imidazo[1,2-b]pyridazine-3-carboxamide (3.46 g, 8.97 mmol, Intermediate OF) in 1,4-dioxane (12 mL) were added K₂CO₃ (3.38 g, 24.5 mmol), RuPhos (0.76 g, 1.63 mmol) and RuPhos Pd G2 (1.27 g, 1.63 mmol) at rt under nitrogen atmosphere. The resulting mixture was stirred at 100° C. for 4 h under nitrogen atmosphere. On completion, the mixture was cooled to rt and concentrated under reduced pressure. The residue was purified by silica gel column chromatography, eluted with CH₂Cl₂/EA (10:1˜1:2), to afford the title compound (3.2 g, 79% yield) as a white solid. LC/MS (ESI, m/z): [(M+1)]⁺=497.2.

Step 2—N-cyclopropyl-6-(4-formyl-2,3-dihydroindol-1-yl)-8-(methylamino)imidazo[1,2-b]pyridazine-3-carboxamide. To a stirred mixture of N-cyclopropyl-6-(4-formyl-2,3-dihydroindol-1-yl)-8-{[(4-methoxyphenyl)methyl](methyl)amino}imidazo[1,2-b]pyridazine-3-carboxamide (3.2 g, 6.4 mmol) in CH₂Cl₂ (30 mL) was added TFA (15 mL) at rt. The resulting mixture was stirred at rt for 1 h. On completion, the mixture was basified to pH 8 with saturated Na₂CO₃ (aq.), and extracted with EtOAc (3×200 mL). The combined organic layers were dried over anhydrous Na₂SO₄. After filtration, the filtrate was concentrated under reduced pressure to afford the title compound (2.8 g) as an off-white solid. LC/MS (ESI, m/z): [(M+1)]⁺=377.2.

### 3-(1-Benzyl-3,3-difluoropiperidin-4-yl)-3,9-diazaspiro[5.5]undecane trifluoroacetate (Intermediate QH)

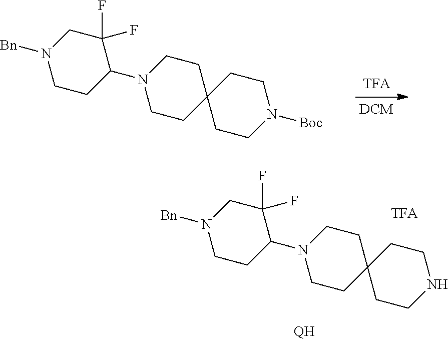

To a stirred solution of tert-butyl 9-(1-benzyl-3,3-difluoropiperidin-4-yl)-3,9-diazaspiro[5.5]undecane-3-carboxylate (320 mg, 0.690 mmol, synthesized via Step 1 of Intermediate MK) in DCM (8 mL) was added TFA (3 mL) at rt under nitrogen atmosphere and the mixture was stirred for 2 h at rt. On completion, the mixture was concentrated under reduced pressure. The residue was purified by trituration with Et₂O (20 mL) to afford the title compound (575 mg) as a brown solid. LC/MS (ESI, m/z): [(M+H)]=364.2.

### 3-{5-[9-(3,3-Difluoropiperidin-4-yl)-3,9-diazaspiro[5.5]undecan-3-yl]-3-methyl-2-oxo-1,3-benzodiazol-1-yl}piperidine-2,6-dione (Intermediate QI)

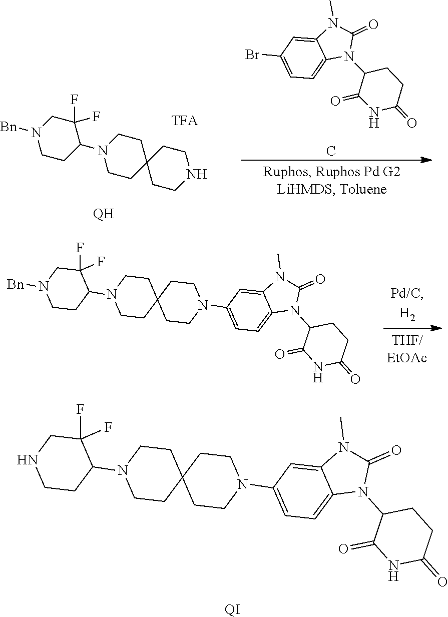

Step 1—3-{5-[9-(1-Benzyl-3,3-difluoropiperidin-4-yl)-3,9-diazaspiro[5.5]undecan-3-yl]-3-methyl-2-oxo-1,3-benzodiazol-1-yl}piperidine-2,6-dione. To a stirred mixture of 3-(1-benzyl-3,3-difluoropiperidin-4-yl)-3,9-diazaspiro[5.5]undecane 2,2,2-trifluoroacetate (300 mg, 0.650 mmol, Intermediate QH) and 3-(5-bromo-3-methyl-2-oxo-1,3-benzodiazol-1-yl)piperidine-2,6-dione (220 mg, 0.650 mmol, Intermediate C) in toluene (5 mL) were added RuPhos (61 mg, 0.130 mmol) and RuPhos-PdCl-2nd G (101 mg, 0.130 mmol) at it under nitrogen atmosphere. To the above mixture was added LiHMDS (3.90 mL, 1.0 mmol/L in THF) at rt then the mixture was stirred for additional 2 h at 80° C. On completion, the mixture was cooled to rt and concentrated under reduced pressure. The residue was dissolved in DMSO (10 mL). The crude product was purified by reverse phase flash (Column: Spherical C18 Column, 20-40 μm, 330 g; Mobile Phase A: Water (0.1% NH₄HCO₃), Mobile Phase B: ACN; Flow rate: 80 mL/min; Gradient: 60% B to 85% B in 25 min, 254 nm; the fractions containing the desired product were collected at 70% B) to afford the title compound (200 mg, 49% yield) as a white solid. LC/MS (ESI, m/z): [(M+H)]⁺=621.3.

Step 2—3-{5-[9-(3,3-Difluoropiperidin-4-yl)-3,9-diazaspiro[5.5]undecan-3-yl]-3-methyl-2-oxo-1,3-benzodiazol-1-yl}piperidine-2,6-dione. To a stirred solution of 3-{5-[9-(1-benzyl-3,3-difluoropiperidin-4-yl)-3,9-diazaspiro[5.5]undecan-3-yl]-3-methyl-2-oxo-1,3-benzodiazol-1-yl}piperidine-2,6-dione (200 mg, 0.322 mmol) in THF (10 mL) and EtOAc (20 mL) was added Pd/C (286 mg, 2.68 mmol) at rt under nitrogen atmosphere. The resulting mixture was stirred overnight at 50° C. under hydrogen atmosphere. On completion, the mixture was cooled to rt and filtered, and the filter cake was washed with DCM (3×30 mL). The filtrate was concentrated under reduced pressure to give the title compound (130 mg, 76% yield) as a white solid. LC/MS (ESI, m/z): [(M+H)]⁺=531.3.

### 4-[4-(1,3-Dioxolan-2-yl)pyridin-2-yl]-2,3-dihydro-1H-indole (Intermediate QJ)

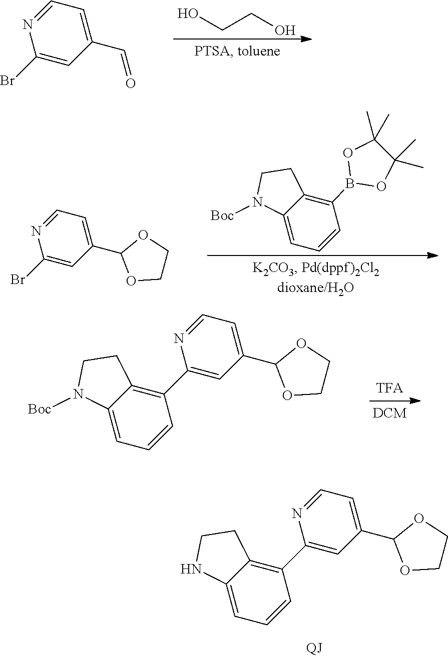

Step 1—2-Bromo-4-(1,3-dioxolan-2-yl)pyridine. To a stirred solution of 2-bromopyridine-4-carbaldehyde (30 g, 160 mmol) and PTSA (2.78 g, 16.1 mmol) in toluene (600 mL) was added ethylene glycol (40.04 g, 645.1 mmol) in portions at rt under nitrogen atmosphere. The resulting mixture was stirred reflux for 2 h under nitrogen atmosphere. On completion, the mixture was cooled to rt and was basified to pH 8 with saturated NaHCO₃ (aq.) and extracted with EtOAc (3×100 mL). The combined organic layers were dried over anhydrous Na₂SO₄. After filtration, the filtrate was concentrated under reduced pressure. The residue was purified by silica gel column chromatography, eluted with PE/EA (1:1) to afford the title compound (30 g, 81% yield) as a yellow oil. LC/MS (ESI, m/z): [(M+1)]⁺=229.0, 231.0.

Step 2—Tert-butyl 4-[4-(1,3-dioxolan-2-yl)pyridin-2-yl]-2,3-dihydroindole-1-carboxylate. To a stirred solution of 2-bromo-4-(1,3-dioxolan-2-yl)pyridine (6 g, 26 mmol) and tert-butyl 4-(4,4,5,5-tetramethyl-1,3,2-dioxaborolan-2-yl)-2,3-dihydroindole-1-carboxylate (12.61 g, 36.51 mmol, synthesized via Step 1 of Intermediate RQ) in dioxane (60 mL) and H₂O (12 mL) were added Pd(dppf)Cl₂ CH₂Cl₂ (2.12 g, 2.61 mmol) and K₂CO₃ (7.21 g, 52.2 mmol) in portions at 80° C. under nitrogen atmosphere. The resulting mixture was stirred for 2 h at 80° C. under nitrogen atmosphere. On completion, the mixture was cooled to rt and concentrated under reduced pressure. The residue was purified by reverse phase flash chromatography (Column: WelFlash™ C18-I, 20-40 μm, 120 g; Eluent A: Water (10 mmol/L NH₄HCO₃); Eluent B: ACN; Gradient: 45%-75% B in 40 min; Flow rate: 80 mL/min; Detector: 220/254 nm; desired fractions were collected at 56% B and concentrated under reduced pressure) to afford the title compound (4.1 g, 43% yield) as a yellow oil. LC/MS (ESI, m/z): [(M+1)]=369.2.

Step 3—4-[4-(1,3-Dioxolan-2-yl)pyridin-2-yl]-2,3-dihydro-1H-indole. To a stirred solution of tert-butyl 4-[4-(1,3-dioxolan-2-yl)pyridin-2-yl]-2,3-dihydroindole-1-carboxylate (4.1 g, 11 mmol) in DCM (60 mL) was added HCl (gas) in 1,4-dioxane (20 mL) dropwise at rt under nitrogen atmosphere. The resulting mixture was stirred for 1 h at rt under nitrogen atmosphere. On completion, the mixture was concentrated under vacuum. The residue was triturated with Et₂O to afford the title compound (2.9 g, 97% yield) as a yellow solid. LC/MS (ESI, m/z): [(M+1)]⁺=269.2.

### 6-[4-(4-Formylpyridin-2-yl)-2,3-dihydroindol-1-yl]-N-[(1S,2S)-2-methoxycyclobutyl]-8-(methylamino)imidazo[1,2-b]pyridazine-3-carboxamide (Intermediate QK)

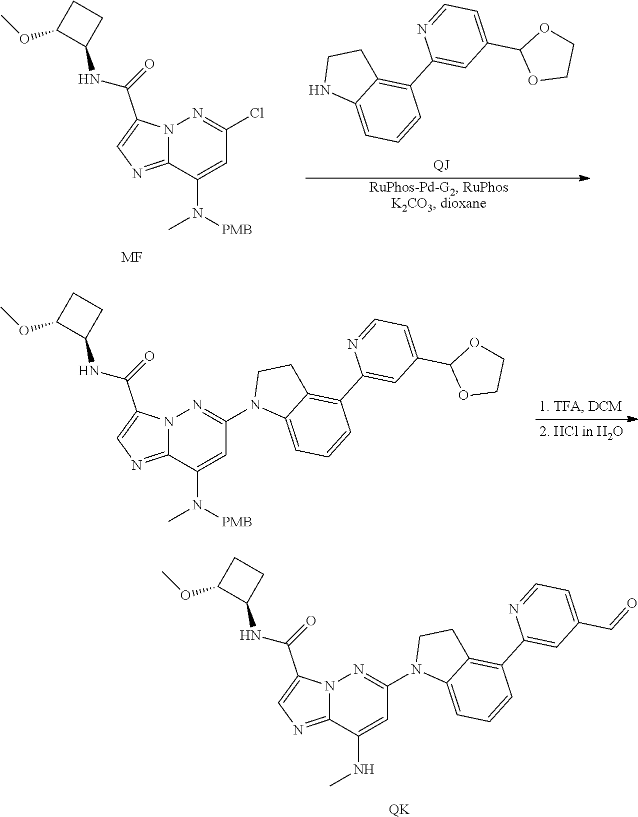

Step 1—6-{4-[4-(1,3-Dioxolan-2-yl)pyridin-2-yl]-2,3-dihydroindol-1-yl}-N-[(1R,2R)-2-methoxycyclobutyl]-8-{[(4-methoxyphenyl)methyl](methyl)amino}imidazo[1,2-b]pyridazine-3-carboxamide. To a stirred solution of 6-chloro-N-[(1R,2R)-2-methoxycyclobutyl]-8-{[(4-methoxyphenyl)methyl](methyl)amino}imidazo[1,2-b]pyridazine-3-carboxamide (300 mg, 0.698 mmol, Intermediate MF) and 4-[4-(1,3-dioxolan-2-yl)pyridin-2-yl]-2,3-dihydro-1H-indole (243 mg, 0.907 mmol, Intermediate QJ) in dioxane (13 mL) were added K₂CO₃ (800 mg, 5.79 mmol), RuPhos (65 mg, 0.14 mmol) and RuPhos-PdCl-2nd G (108 mg, 0.140 mmol) at rt under nitrogen atmosphere. The resulting mixture was stirred for 2 h at 100° C. under nitrogen atmosphere. On completion, the mixture was cooled to rt and dissolved in H₂O (50 mL). The resulting mixture was extracted with EtOAc (3×100 mL). The combined organic layers were washed with brine (3×200 mL), dried over anhydrous Na₂SO₄, filtered, and the filtrate was concentrated under reduced pressure. The crude product was purified by reverse phase flash (Column: Spherical C18 Column, 20-40 μm, 330 g; Mobile Phase A: Water (0.1% NH₄HCO₃), Mobile Phase B: ACN; Flow rate: 80 mL/min; Gradient: 65% B to 85% B in 25 min, 254 nm; the fractions containing the desired product were collected at 83% B) to afford the title compound (250 mg, 54% yield) as a white solid. LC/MS (ESI, m/z): [(M+H)]⁺=662.4.

Step 2—6-[4-(4-Formylpyridin-2-yl)-2,3-dihydroindol-1-yl]-N-[(1S,2S)-2-methoxycyclobutyl]-8-(methylamino)imidazo[1,2-b]pyridazine-3-carboxamide. A solution of 6-{4-[4-(1,3-dioxolan-2-yl)pyridin-2-yl]-2,3-dihydroindol-1-yl}-N-[(1S,2S)-2-methoxycyclobutyl]-8-{[(4-methoxyphenyl)methyl](methyl)amino}imidazo[1,2-b]pyridazine-3-carboxamide (260 mg, 0.393 mmol) in TFA (7 mL) and DCM (21 mL) was stirred for 2 h at rt. Then, the mixture was concentrated under reduced pressure. The residue was dissolved in 3NHCl (20 mL) and the mixture was stirred for overnight at 50° C. On completion, the mixture was cooled to rt and basified to pH 9 with saturated Na₂CO₃ (aq.). The resulting mixture was extracted with EtOAc (3×100 mL). The combined organic layers were washed with brine (1×200 mL), dried over anhydrous Na₂SO₄. After filtration, the filtrate was concentrated under reduced pressure to give the title compound (120 mg, 61% yield) as a yellow solid. LC/MS (ESI, m/z): [(M+H)]+=498.2.

### 3-(5-(4-(Azetidin-3-ylmethyl)piperazin-1-yl)-3-methyl-2-oxo-2,3-dihydro-1H-benzo[d]imidazol-1-yl)piperidine-2,6-dione trifluoroacetate (Intermediate QL)

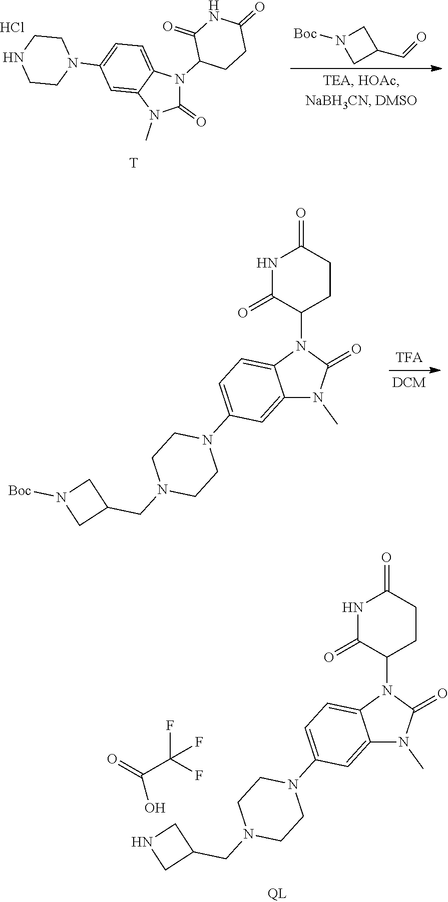

Step 1—Tert-butyl 3-((4-(1-(2,6-dioxopiperidin-3-yl)-3-methyl-2-oxo-2,3-dihydro-1H-benzo[d]imidazol-5-yl)piperazin-1-yl)methyl)azetidine-1-carboxylate. To a stirred solution of 3-(3-methyl-2-oxo-5-(piperazin-1-yl)-2,3-dihydro-1H-benzo[d]imidazol-1-yl)piperidine-2,6-dione hydrochloride (1.00 g, 2.91 mmol, Intermediate T) in DMSO (10 mL) was added KOAc (1.14 g, 11.62 mmol) at rt under nitrogen atmosphere and the mixture was stirred for 5 min at rt. The mixture was basified to pH 8. To the above mixture was added HOAc (525 mg, 8.736 mmol) and tert-butyl 3-formylazetidine-1-carboxylate (647 mg, 3.50 mmol, CAS #177947-96-5) at rt. The resulting mixture was stirred for an additional 30 min at 50° C. To the above mixture was added NaBH₃CN (549 mg, 8.736 mmol) in portions over 5 min at rt. The resulting mixture was stirred for 2 h at 50° C. under nitrogen atmosphere. On completion, the mixture was cooled to rt and purified by reverse phase flash chromatography (Column: WelFlash™ C18-I, 20-40 m, 330 g; Eluent A: Water (plus 5 mmol/L FA); Eluent B: ACN; Gradient: 30%-50% B in 20 min; Flow rate: 80 mL/min; Detector: 220/254 nm; desired fractions were collected at 37% B and concentrated under reduced pressure) to afford the title compound (800 mg, 54% yield) as a white solid. LC/MS (ESI, m/z): [(M+H)]⁺=513.3.

Step 2—3-(5-(4-(Azetidin-3-ylmethyl)piperazin-1-yl)-3-methyl-2-oxo-2,3-dihydro-1H-benzo[d]imidazol-1-yl)piperidine-2,6-dione trifluoroacetate. To a stirred solution of tert-butyl 3-((4-(1-(2,6-dioxopiperidin-3-yl)-3-methyl-2-oxo-2,3-dihydro-1H-benzo[d]imidazol-5-yl)piperazin-1-yl)methyl)azetidine-1-carboxylate (800 mg, 1.56 mmol) in DCM (12 mL) was added TFA (4 mL) at rt and the mixture was stirred for 1 h at rt under nitrogen atmosphere. On completion, the mixture was concentrated under reduced pressure. The residue was purified by trituration with Et₂O (30 mL) and dried to give the title compound (600 mg, 75% yield) as a white solid. LC/MS (ESI, m/z): [(M+H)]⁺=413.2.

### 3-(5-(4-((1-(3,3-Difluoropiperidin-4-yl)azetidin-3-yl)methyl)piperazin-1-yl)-3-methyl-2-oxo-2,3-dihydro-LH-benzo[d]imidazol-1-yl)piperidine-2,6-dione trifluoroacetate (Intermediate QM)

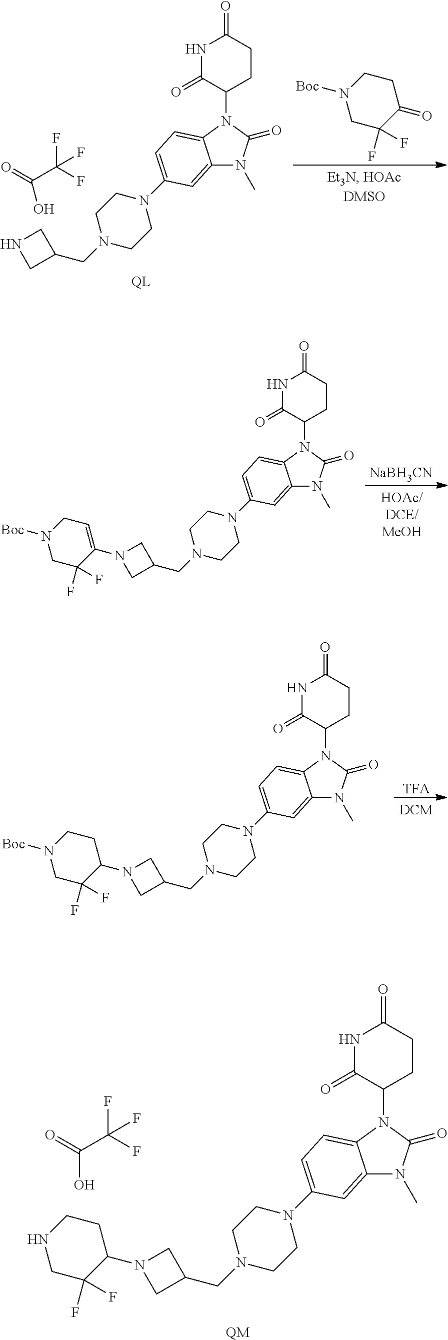

Step 1—Tert-butyl 4-(3-((4-(1-(2,6-dioxopiperidin-3-yl)-3-methyl-2-oxo-2,3-dihydro-1H-benzo[d]imidazol-5-yl)piperazin-1-yl)methyl)azetidin-1-yl)-3,3-difluoro-3,6-dihydropyridine-1(2H)-carboxylate. To a stirred solution of 3-(5-(4-(azetidin-3-ylmethyl)piperazin-1-yl)-3-methyl-2-oxo-2,3-dihydro-TH-benzo[d]imidazol-1-yl)piperidine-2,6-dione trifluoroacetate (400 mg, 0.970 mmol, Intermediate QL) in DMSO (8 mL) was added TEA (147 mg, 1.455 mmol) at rt under nitrogen atmosphere and the mixture was stirred for 5 min at rt. The mixture was basified to pH 8. To the above mixture was added tert-butyl 3,3-difluoro-4-oxopiperidine-1-carboxylate (342 mg, 1.455 mmol, CAS #1400264-85-8) and HOAc (175 mg, 2.91 mmol) at rt. The resulting mixture was stirred for overnight at 100° C. under nitrogen atmosphere. On completion, the mixture was cooled to rt and diluted with water (200 mL). The aqueous layer was extracted with EtOAc (3×100 mL). The combined organic layers were washed with brine (300 mL), dried over anhydrous Na₂SO₄. After filtration, the filtrate was concentrated under reduced pressure to give the title compound (2.00 g) as a yellow oil. LC/MS (ESI, m/z): [(M+H)]f=630.3.

Step 2—Tert-butyl 4-(3-((4-(1-(2,6-dioxopiperidin-3-yl)-3-methyl-2-oxo-2,3-dihydro-1H-benzo[d]imidazol-5-yl)piperazin-1-yl)methyl)azetidin-1-yl)-3,3-difluoropiperidine-1-carboxylate. To a stirred solution of tert-butyl 4-(3-((4-(1-(2,6-dioxopiperidin-3-yl)-3-methyl-2-oxo-2,3-dihydro-1H-benzo[d]imidazol-5-yl)piperazin-1-yl)methyl)azetidin-1-yl)-3,3-difluoro-3,6-dihydropyridine-1(2H)-carboxylate (500 mg, 0.794 mmol) in DCE (10 mL) and MeOH (10 mL) were added HOAc (1 mL) and NaBH₃CN (249 mg, 3.970 mmol) at rt and the mixture was stirred overnight at rt under nitrogen atmosphere. On completion, the mixture was concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 20%-40% B in 25 min; Flow rate: 80 mL/min; Detector: 220/254 nm; desired fractions were collected at 38% B and concentrated under reduced pressure) to afford the title compound (400 mg, 80% yield) as an off-white solid. LC/MS (ESI, m/z): [(M+H)]f=632.3.

Step 3—3-(5-(4-((1-(3,3-Difluoropiperidin-4-yl)azetidin-3-yl)methyl)piperazin-1-yl)-3-methyl-2-oxo-2,3-dihydro-1H-benzo[d]imidazol-1-yl)piperidine-2,6-dione trifluoroacetate. To a stirred solution of tert-butyl 4-(3-((4-(1-(2,6-dioxopiperidin-3-yl)-3-methyl-2-oxo-2,3-dihydro-1H-benzo[d]imidazol-5-yl)piperazin-1-yl)methyl)azetidin-1-yl)-3,3-difluoropiperidine-1-carboxylate (400 mg, 0.633 mmol) in DCM (9 mL) was added TFA (3 mL) at rt and the mixture was stirred for 1 h at rt under nitrogen atmosphere. On completion, the mixture was concentrated under reduced pressure. The residue was purified by trituration with Et₂O (30 mL) and dried under reduced pressure to afford the title compound (300 mg, 75% yield) as a brown oil. LC/MS (ESI, m/z): [(M+H)]⁺=532.3.

### 1-(1-(1-Benzyl-3,3-difluoropiperidin-4-yl)azetidin-3-yl)piperazine (Intermediate QN)

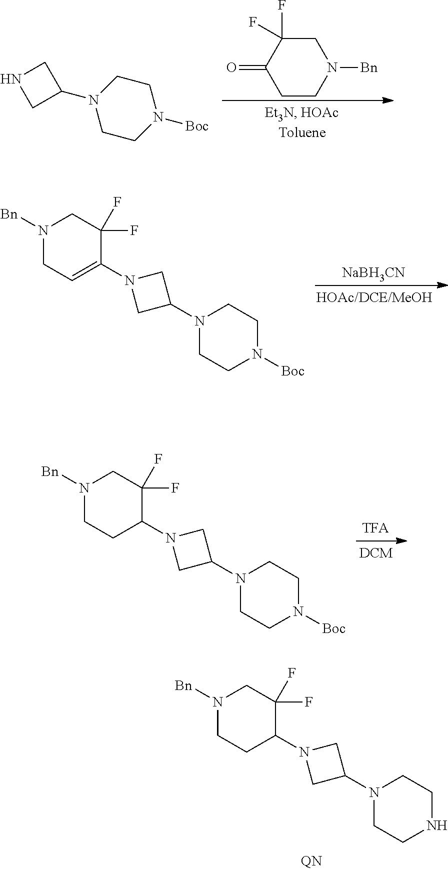 [SMILES: CC(C)(C)OC(=O)N1CCN(C2CNC2)CC1 ; O=C1CCN(Cc2ccccc2)CC1(F)F ; CC(C)(C)OC(=O)N1CCN(C2CN(C3=CCN(Cc4ccccc4)CC3(F)F)C2)CC1 ; "NaBH3CN" ; CC(C)(C)OC(=O)N1CCN(C2CN(C3CCN(Cc4ccccc4)CC3(F)F)C2)CC1 ; FC1(F)CN(Cc2ccccc2)CCC1N1CC(N2CCNCC2)C1]

Step 1—Tert-butyl 4-(1-(1-benzyl-3,3-difluoro-1,2,3,6-tetrahydropyridin-4-yl)azetidin-3-yl)piperazine-1-carboxylate. To a stirred solution of tert-butyl 4-(azetidin-3-yl)piperazine-1-carboxylate (5.00 g, 20.7 mmol, CAS #178312-58-8) in toluene (50 mL) was added TEA (3.14 g, 31.1 mmol) at it under nitrogen atmosphere and the mixture was stirred for 5 min at rt. The mixture was basified to pH 8. To the above mixture was added 1-benzyl-3,3-difluoropiperidin-4-one (7.00 g, 31.1 mmol, CAS #1039741-54-2) and HOAc (3.73 g, 62.1 mmol) at rt. The resulting mixture was stirred for 3 h at 110° C. under nitrogen atmosphere. On completion, the mixture was cooled to rt and neutralized to pH 7 with saturated NaHCO₃ (aq.). The aqueous layer was extracted with EtOAc (3×300 mL). The combined organic layers were washed with brine (600 mL), dried over anhydrous Na₂SO₄. After filtration, the filtrate was concentrated under reduced pressure to afford the title compound (10.00 g) as a brown oil. LC/MS (ESI, m/z): [(M+H)]+=449.2.

Step 2—Tert-butyl 4-(1-(1-benzyl-3,3-difluoropiperidin-4-yl)azetidin-3-yl)piperazine-1-carboxylate. To a stirred solution of tert-butyl 4-(1-(1-benzyl-3,3-difluoro-1,2,3,6-tetrahydropyridin-4-yl)azetidin-3-yl)piperazine-1-carboxylate (10.00 g, 22.29 mmol) in DCE (10 mL) and MeOH (10 mL) were added AcOH (1 mL) and NaBH₃CN (4.48 g, 71.34 mmol) at it and the mixture was stirred for overnight at rt under nitrogen atmosphere. On completion, the mixture was concentrated under reduced pressure. The crude product was purified by silica gel column chromatography (Mobile Phase A: CH₂C2, Mobile Phase B: MeOH; Gradient: 0% B to 20% B in 20 min, 254 nm; the fractions containing the desired product were collected at 16% B) to afford the title compound (2.80 g, 28% yield) as a yellow oil. LC/MS (ESI, m/z): [(M+H)]⁺=451.3.

Step 3—1-(1-(1-Benzyl-3,3-difluoropiperidin-4-yl)azetidin-3-yl)piperazine. To a stirred solution of tert-butyl 4-(1-(1-benzyl-3,3-difluoropiperidin-4-yl)azetidin-3-yl)piperazine-1-carboxylate (1.00 g, 2.22 mmol) in DCM (12 mL) was added TFA (4 mL) at rt under nitrogen atmosphere and the mixture was stirred for 1 h at rt under nitrogen atmosphere. On completion, the mixture was basified to pH 8 with saturated NH₄HCO₃ (aq.). The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 20%-40% B in 20 min; Flow rate: 80 mL/min; Detector: 220/254 nm; desired fractions were collected at 36% B and concentrated under reduced pressure) to afford the title compound (430 mg, 55% yield) as a white oil. LC/MS (ESI, m/z): [(M+H)]⁺=351.3.

### 3-(5-(4-(1-(3,3-Difluoropiperidin-4-yl)azetidin-3-yl)piperazin-1-yl)-3-methyl-2-oxo-2,3-dihydro-1H-benzo[d]imidazol-1-yl)piperidine-2,6-dione (Intermediate QO)

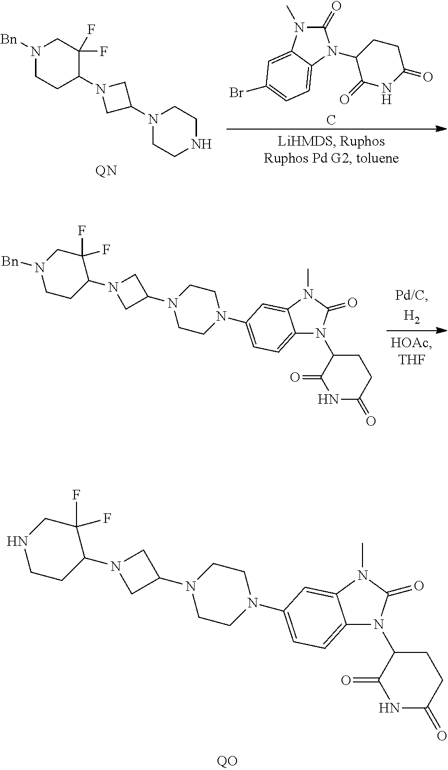

Step 1—3-(5-(4-(1-(1-Benzyl-3,3-difluoropiperidin-4-yl)azetidin-3-yl)piperazin-1-yl)-3-methyl-2-oxo-2,3-dihydro-1H-benzo[d]imidazol-1-yl)piperidine-2,6-dione. To a stirred solution of 1-(1-(1-benzyl-3,3-difluoropiperidin-4-yl)azetidin-3-yl)piperazine (400 mg, 1.14 mmol, Intermediate QN) and 3-(5-bromo-3-methyl-2-oxo-2,3-dihydro-1H-benzo[d]imidazol-1-yl)piperidine-2,6-dione (386 mg, 1.14 mmol, Intermediate C) in toluene (10 mL) were added RuPhos (107 mg, 0.228 mmol) and RuPhos-PdCl-2nd G (178 mg, 0.228 mmol) at rt under nitrogen atmosphere. To the above mixture was added LiHMDS (9.14 ml) dropwise over 5 min at rt under nitrogen atmosphere. The resulting mixture was stirred for an additional 2 h at 80° C. On completion, the mixture was cooled to rt and the mixture was concentrated under reduced pressure. The residue was purified by reverse phase flash chromatography (Column: WelFlash™ C18-I, 20-40 μm, 120 g; Eluent A: Water (plus 10 mmol/L FA); Eluent B: ACN; Gradient: 30%-50% B in 25 min; Flow rate: 80 mL/min; Detector: 220/254 nm; desired fractions were collected at 46% B and concentrated under reduced pressure) to afford the title compound (400 mg, 58% yield) as a white solid. LC/MS (ESI, m/z): [(M+H)]⁺=608.3.

Step 2—3-(5-(4-(1-(3,3-Difluoropiperidin-4-yl)azetidin-3-yl)piperazin-1-yl)-3-methyl-2-oxo-2,3-dihydro-TH-benzo[d]imidazol-1-yl)piperidine-2,6-dione. To a stirred solution of 3-(5-(4-(1-(1-benzyl-3,3-difluoropiperidin-4-yl)azetidin-3-yl)piperazin-1-yl)-3-methyl-2-oxo-2,3-dihydro-1H-benzo[d]imidazol-1-yl)piperidine-2,6-dione (400 mg, 0.658 mmol) in THF (20 mL) and HOAc (0.04 mL) was added 10 wt % Pd/C (7 mg, 0.066 mmol) at rt under nitrogen atmosphere. The reaction system was degassed under vacuum and purged with H₂ several times, Then the mixture was hydrogenated under H₂ balloon (1 atm) at 25° C. for 16 h at rt under nitrogen atmosphere. After completion of the reaction, the Pd/C was filtered off through celite and the corresponding filtrate was concentrated under reduced pressure to afford the title compound (100 mg, 29% yield) as a white solid. LC/MS (ESI, m/z): [(M+H)]f=518.4.

### 6-(4-(3-Bromoprop-1-yn-1-yl)indolin-1-yl)-N-((1R,2S)-2-fluorocyclopropyl)-8-(methylamino)imidazo[1,2-b]pyridazine-3-carboxamide (Intermediate QP)

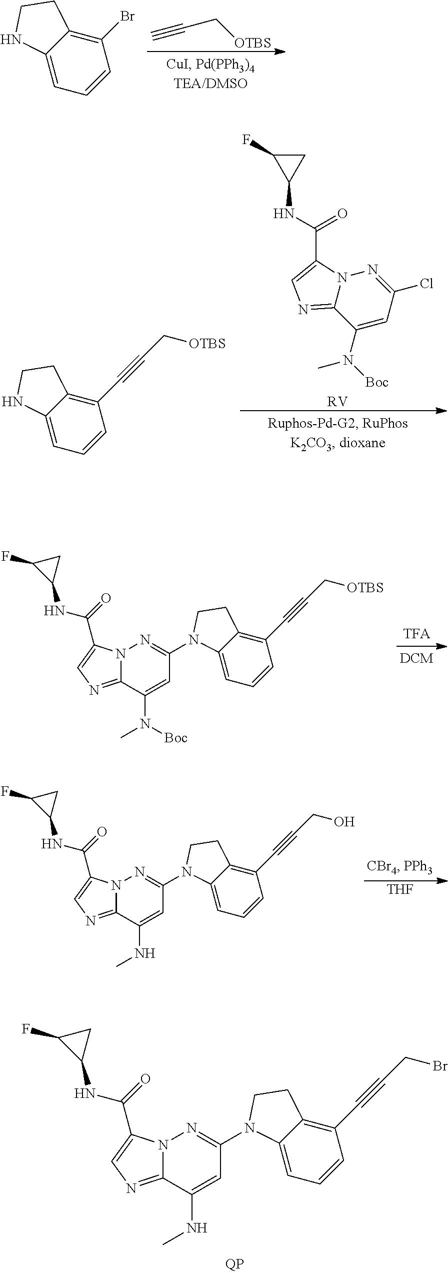

Step 1—4-{3-[(Tert-butyldimethylsilyl)oxy]prop-1-yn-1-yl}-2,3-dihydro-1H-indole. To a stirred mixture of 4-bromo-2,3-dihydro-1H-indole (5.00 g, 25.2 mmol) and tert-butyldimethyl(prop-2-yn-1-yloxy)silane (4.30 g, 25.2 mmol) in TEA (40 mL) and DMSO (60 mL) were added CuI (0.96 g, 5.05 mmol) and Pd(PPh₃)₄(2.92 g, 2.52 mmol) at rt under nitrogen atmosphere. The resulting mixture was stirred for 2 h at 80° C. On completion, the mixture was concentrated under reduced pressure. The mixture was purified by reverse phase Flash chromatography (Column: WelFlash™ C18-I, 20-40 μm, 120 g; Eluent A: Water (plus 10 mmol/L NH₄HCO₃); Eluent B: ACN; Gradient: 75%-95% B in 25 min; Flow rate: 80 mL/min; Detector: 220/254 nm; desired fractions were collected at 95% B and concentrated under reduced pressure) to afford the title compound (3.5 g, 48% yield) as a brown oil. LC/MS (ESI, m/z): [(M+1)]⁺=288.2.

Step 2—6-(4-{3-[(Tert-butyldimethylsilyl)oxy]prop-1-yn-1-yl}-2,3-dihydroindol-1-yl)-N-[(1R,2S)-2-fluorocyclopropyl]-8-{[(4-methoxyphenyl)methyl](methyl)amino}imidazo[1,2-b]pyridazine-3-carboxamide. To a stirred mixture of 4-{3-[(tert-butyldimethylsilyl)oxy]prop-1-yn-1-yl}-2,3-dihydro-1H-indole (3.00 g, 10.4 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (4.21 g, 10.4 mmol, Intermediate RV) in dioxane (60 mL) were added K₂CO₃ (4.33 g, 31.3 mmol), RuPhos (0.97 g, 2.09 mmol) and RuPhos-PdCl-2nd G (1.62 g, 2.09 mmol) at rt under nitrogen atmosphere. The resulting mixture was stirred for 3 h at 100° C. On completion, the mixture was concentrated under reduced pressure. The residue was purified by silica gel column chromatography, eluted with CH₂Cl₂/EA (1:1), to afford the title compound (3.3 g, 48% yield) as a brown solid. LC/MS (ESI, m/z): [(M+1)]⁺=635.3.

Step 3—N-[(1R,2S)-2-fluorocyclopropyl]-6-[4-(3-hydroxyprop-1-yn-1-yl)-2,3-dihydroindol-1-yl]-8-(methylamino)imidazo[1,2-b]pyridazine-3-carboxamide. To a stirred mixture of 6-(4-{3-[(tert-butyldimethylsilyl)oxy]prop-1-yn-1-yl}-2,3-dihydroindol-1-yl)-N-[(1R,2S)-2-fluorocyclopropyl]-8-{[(4-methoxyphenyl)methyl](methyl)amino}imidazo[1,2-b]pyridazine-3-carboxamide (3.00 g, 4.58 mmol) in DCM (30 mL) was added TFA (30 mL) at rt and the mixture was stirred for 1 h at rt. On completion, the mixture was concentrated under reduced pressure. The mixture was basified to pH 9 with TEA. The mixture was purified by reverse phase flash chromatography (Column: WelFlash™ C18-I, 20-40 μm, 120 g; Eluent A: Water (plus 10 mmol/L NH₄HCO₃); Eluent B: ACN; Gradient: 40%-60% B in 25 min; Flow rate: 80 mL/min; Detector: 220/254 nm; desired fractions were collected at 48% B and concentrated under reduced pressure) to afford the title compound (550 mg, 28% yield) as a yellow solid. LC/MS (ESI, m/z): [(M+1)]⁺=421.2.

Step 4—6-[4-(3-Bromoprop-1-yn-1-yl)-2,3-dihydroindol-1-yl]-N-[(1R,2S)-2-fluorocyclopropyl]-8-(methylamino)imidazo[1,2-b]pyridazine-3-carboxamide. To a stirred solution of N-[(1R,2S)-2-fluorocyclopropyl]-6-[4-(3-hydroxyprop-1-yn-1-yl)-2,3-dihydroindol-1-yl]-8-(methylamino)imidazo[1,2-b]pyridazine-3-carboxamide (400 mg, 0.951 mmol) in THF (6 mL) were added CBr₄ (480 mg, 1.45 mmol) and PPh₃ (400 mg, 1.53 mmol) at rt and the mixture was stirred for 2 h at rt under nitrogen atmosphere. The resulting mixture was concentrated under reduced pressure. The residue was purified by silica gel column chromatography, eluted with CH₂Cl₂/MeOH (10:1), to afford the title compound (120 mg, 26% yield) as a white solid. LC/MS (ESI, m/z): [(M+1)]⁺=483.1, 485.1.

### 1-(2-chloro-5-(4-(piperazin-1-yl)piperidine-1-carbonyl)phenyl)dihydropyrimidine-2,4(1H,3H)-dione 2,2,2-trifluoroacetate (Intermediate QQ)

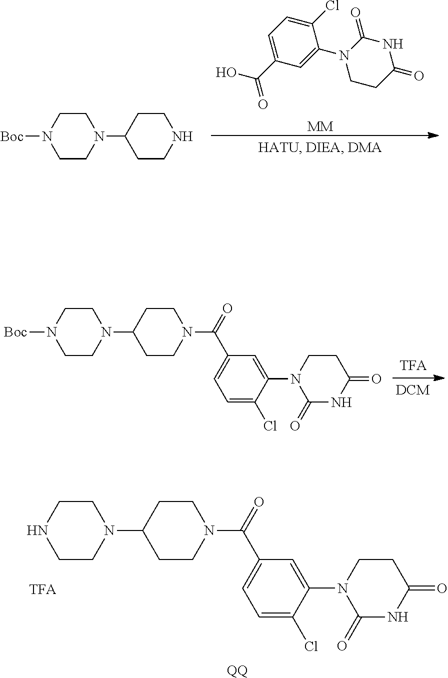

Step 1—Tert-butyl 4-(1-(4-chloro-3-(2,4-dioxotetrahydropyrimidin-1(2H)-yl)benzoyl)piperidin-4-yl)piperazine-1-carboxylate. To a stirred solution of 4-chloro-3-(2,4-dioxo-1,3-diazinan-1-yl)benzoic acid (360 mg, 1.34 mmol, Intermediate MM) in DMA (5 mL) were added HATU (600 mg, 1.578 mmol) and DIEA (420 mg, 3.250 mmol) at rt and the mixture was stirred for 5 min at rt. To the above mixture was added tert-butyl 4-(piperidin-4-yl)piperazine-1-carboxylate (300 mg, 1.11 mmol, CAS #205059-24-1) at rt. The resulting mixture was stirred for an additional 1 h at rt. On completion, the solution was purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 35%-65% B in 35 min; Flow rate: 80 mL/min; Detector: 220/254 nm; desired fractions were collected at 52% B and concentrated under reduced pressure) to afford the title compound (400 mg, 69% yield) as a brown solid. LC/MS (ESI, m/z): [(M+1)]⁺=520.0

Step 2—1-(2-Chloro-5-(4-(piperazin-1-yl)piperidine-1-carbonyl)phenyl)dihydropyrimidine-2,4(1H,3H)-dione 2,2,2-trifluoroacetate. To a stirred solution of tert-butyl 4-{1-[4-chloro-3-(2,4-dioxo-1,3-diazinan-1-yl)benzoyl]piperidin-4-yl}piperazine-1-carboxylate (400 mg, 0.769 mmol) in DCM (9 mL) was added TFA (3 mL) at rt and the mixture was stirred for 1 h at rt. On completion, the mixture was concentrated under reduced pressure. The residue was purified by trituration with Et₂O (10 mL) to give the title compound (300 mg, TFA salt) as a brown solid. LC/MS (ESI, m/z): [(M+1)]⁺=419.9.

### 5-(2,3-Dihydro-1H-indol-4-yl)pyrazine-2-carbaldehyde (Intermediate QR)

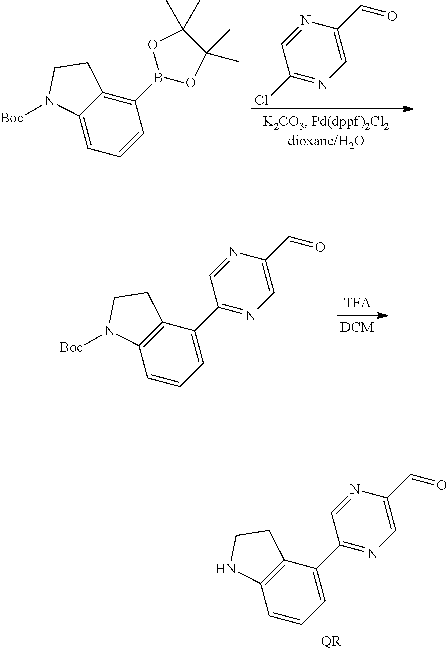

Step 1—Tert-butyl 4-(5-formylpyrazin-2-yl)indoline-1-carboxylate. To a stirred solution of tert-butyl 4-(4,4,5,5-tetramethyl-1,3,2-dioxaborolan-2-yl)-2,3-dihydroindole-1-carboxylate (5.00 g, 14.5 mmol, synthesized via Step 1 of Intermediate RQ) and 5-chloropyrazine-2-carbaldehyde (2.06 g, 14.5 mmol, CAS #88625-24-5) and K₂CO₃ (6.00 g, 43.4 mmol) in dioxane (30 mL) and H₂O (5 mL) was added Pd(dppf)Cl₂ (2.12 g, 2.89 mmol) dropwise at rt under nitrogen atmosphere. The resulting mixture was stirred for 16 h at 90° C. under nitrogen atmosphere. On completion, the mixture was cooled to it and concentrated under reduced pressure. The mixture was purified by silica gel column chromatography (Mobile Phase A: PE, Mobile Phase B: EA; Gradient: 0% to 50% B in 25 min, 254 nm; the fractions containing the desired product were collected at 20% B and concentrated under reduced pressure) to afford the title compound (2.80 g, 59% yield) as a white solid. LC/MS (ESI, m/z): [(M+H)]⁺=326.1.

Step 2—5-(2,3-Dihydro-1H-indol-4-yl)pyrazine-2-carbaldehyde. To a stirred solution of tert-butyl 4-(5-formylpyrazin-2-yl)indoline-1-carboxylate (800 mg, 2.45 mmol) in DCM (12 mL) was added TFA (3 mL, 40 mmol) dropwise at rt under air atmosphere. The resulting mixture was stirred for 1 h at rt under air atmosphere. On completion, the mixture was concentrated under reduced pressure. The mixture was purified by trituration with Et₂O (10 mL) to afford the title compound (500 mg, 90% yield) as a white solid. LC/MS (ESI, m/z): [(M+H)]⁺=226.1.

### Tert-butyl 3-((6-(indolin-4-yl)pyridin-3-yl)methyl)azetidine-1-carboxylate (Intermediate QS)

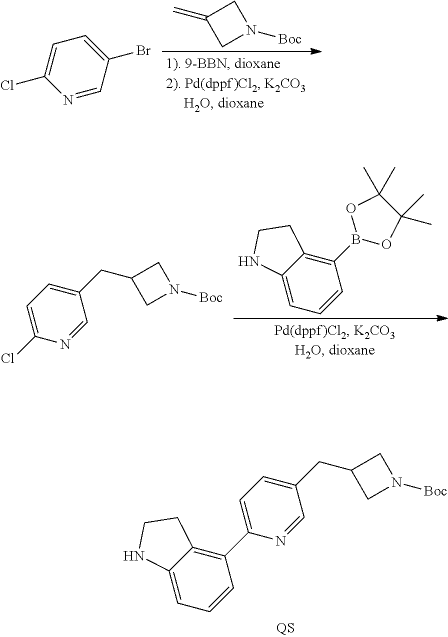 [SMILES: Clc1ccc(Br)cn1 ; C=C1CN(C(=O)OC(C)(C)C)C1 ; CC(C)(C)OC(=O)N1CC(Cc2ccc(Cl)nc2)C1 ; CC1(C)OB(c2cccc3c2CCN3)OC1(C)C ; CC(C)(C)OC(=O)N1CC(Cc2ccc(-c3cccc4c3CCN4)nc2)C1]

Step 1—Tert-butyl 3-((6-chloropyridin-3-yl)methyl)azetidine-1-carboxylate. To a stirred mixture of tert-butyl 3-methyleneazetidine-1-carboxylate (5.00 g, 29.6 mmol) in dioxane (100 mL) was added (1s,5s)-9-[(1s,5s)-9-borabicyclo[3.3.1]nonan-9-yl]-9-borabicyclo[3.3.1]nonane (7.87 g, 32.5 mmol) in portions at rt. The resulting mixture was stirred for 1 h at 50° C. To the above mixture was added 5-bromo-2-chloropyridine (5.69 g, 29.6 mmol), K₂CO₃ (12 g, 89 mmol), H₂O (20 mL) and Pd(dppf)Cl₂·CH₂Cl₂ (2.41 g, 2.95 mmol) at rt under nitrogen atmosphere. The resulting mixture was stirred for 1 h at 90° C. On completion, the mixture was cooled to rt and purified directly by reversed-phase flash chromatography (Column: Spherical C18, 20-40 μm, 330 g; Mobile Phase A: Water (plus 0.1% HCOOH), Mobile Phase B: ACN; Flow rate: 80 mL/min; Gradient (B %): 5%40%, 4 min; 40%˜60%, 20 min; 60%˜95%; 2 min; 95%, 5 min; Detector: 254 nm; the fractions containing desired product were collected at 58% B and concentrated under reduced pressure) to afford the title compound (3.70 g, 44% yield) as a brown solid. LC/MS (ESI, m/z): [(M-56+H)]⁻=227.0.

Step 2—Tert-butyl 3-((6-(indolin-4-yl)pyridin-3-yl)methyl)azetidine-1-carboxylate. To a stirred mixture of tert-butyl 3-((6-chloropyridin-3-yl)methyl)azetidine-1-carboxylate (1.50 g, 5.30 mmol), 4-(4,4,5,5-tetramethyl-1,3,2-dioxaborolan-2-yl)indoline (1.30 g, 5.30 mmol, CAS #388116-27-6) and K₂CO₃ (2.20 g, 15.9 mmol) in dioxane (15 mL) and H₂O (1.5 mL) was added Pd(dppf)Cl₂·CH₂Cl₂ (430 mg, 0.530 mmol) in portions at rt under nitrogen atmosphere. The resulting mixture was stirred for 1 h at 80° C. under nitrogen atmosphere. On completion, the mixture was cooled to rt and diluted with water (100 mL). The resulting mixture was extracted with EtOAc (3×100 mL). The combined organic layers were washed with brine (3×200 mL), dried over anhydrous Na₂SO₄, filtered, and the filtrate was concentrated under reduced pressure. The residue was purified by silica gel column chromatography, eluted with petroleum ether/EtOAc (1:1), to afford the title compound (730 mg, 37% yield) as a brown solid. LC/MS (ESI, m/z): [(M+1)]⁺=366.1.

### 6-(4-(5-(azetidin-3-ylmethyl)pyridin-2-yl)indolin-1-yl)-N-((1R,2S)-2-fluorocyclopropyl)-8-(methylamino)imidazo[1,2-b]pyridazine-3-carboxamide 2,2,2-trifluoroacetate (Intermediate QT)

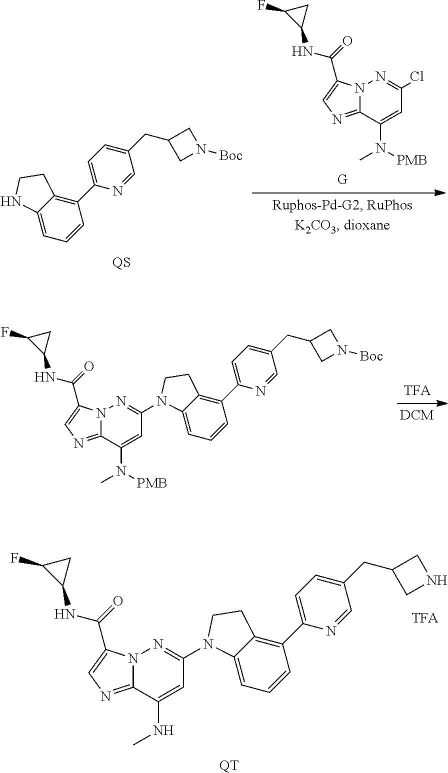

Step 1—Tert-butyl 3-((6-(1-(3-(((1R,2S)-2-fluorocyclopropyl)carbamoyl)-8-((4-methoxybenzyl)(methyl)amino)imidazo[1,2-b]pyridazin-6-yl)indolin-4-yl)pyridin-3-yl)methyl)azetidine-1-carboxylate. To a stirred mixture of tert-butyl 3-((6-(indolin-4-yl)pyridin-3-yl)methyl)azetidine-1-carboxylate (700 mg, 1.92 mmol, Intermediate QS), 6-chloro-N-((1R,2S)-2-fluorocyclopropyl)-8-((4-methoxybenzyl)(methyl)amino)imidazo[1,2-b]pyridazine-3-carboxamide (851 mg, 2.11 mmol, Intermediate G) and K₂CO₃ (794 mg, 5.75 mmol) in dioxane (14 mL) were added RuPhos (89 mg, 0.19 mmol) and RuPhos-PdCl-2nd G (149 mg, 0.192 mmol) in portions at rt under nitrogen atmosphere. The resulting mixture was stirred for 2 h at 100° C. under nitrogen atmosphere. On completion, the mixture was cooled to rt and purified directly by reversed-phase flash chromatography (Column: Spherical C18, 20˜40 m, 330 g; Mobile Phase A: Water (plus 10 mM NH₄HCO₃), Mobile Phase B: ACN; Flow rate: 80 mL/min; Gradient (B %): 5%˜60%, 4 min; 60%˜90%, 20 min; 90%˜95%; 10 min; 95%, 5 min; Detector: 254 nm; the fractions containing desired product were collected at 93% B and concentrated under reduced pressure) to afford the title compound (900 mg, 64% yield) as a brown solid. LC/MS (ESI, m/z): [(M+1)]⁺=733.3.

Step 2—6-(4-(5-(Azetidin-3-ylmethyl)pyridin-2-yl)indolin-1-yl)-N-((1R,2S)-2-fluorocyclopropyl)-8-(methylamino)imidazo[1,2-b]pyridazine-3-carboxamide 2,2,2-trifluoroacetate. A mixture of tert-butyl 3-((6-(1-(3-(((1R,2S)-2-fluorocyclopropyl)carbamoyl)-8-((4-methoxybenzyl)(methyl)amino)imidazo[1,2-b]pyridazin-6-yl)indolin-4-yl)pyridin-3-yl)methyl)azetidine-1-carboxylate (900 mg, 1.23 mmol) in TFA (4 mL) and DCM (12 mL) was stirred for 1 h at rt. On completion, the mixture was concentrated under reduced pressure. The residue was purified by trituration with Et₂O (10 mL) to afford the title compound (600 mg, TFA salt) as a brown solid. LC/MS (ESI, m/z): [(M+1)]⁺=513.3.

### 6-(4-(5-((1-(3,3-Difluoropiperidin-4-yl)azetidin-3-yl)methyl)pyridin-2-yl)indolin-1-yl)-N-((1R,2S)-2-fluorocyclopropyl)-8-(methylamino)imidazo[1,2-b]pyridazine-3-carboxamide trifluoroacetate (Intermediate QU)

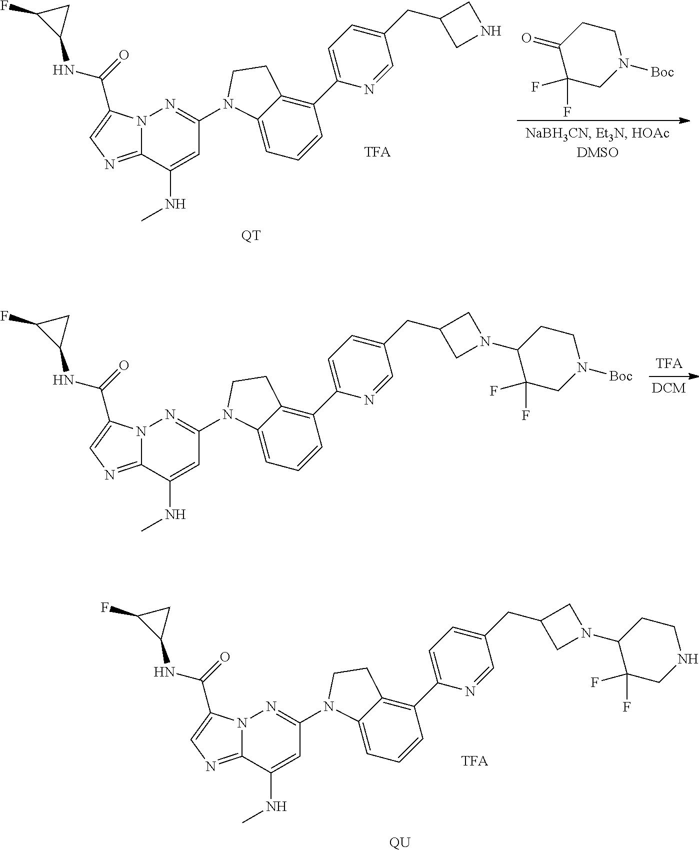

Step 1—Tert-butyl 3,3-difluoro-4-(3-((6-(1-(3-(((1R,2S)-2-fluorocyclopropyl)carbamoyl)-8-(methylamino)imidazo[1,2-b]pyridazin-6-yl)indolin-4-yl)pyridin-3-yl)methyl)azetidin-1-yl)piperidine-1-carboxylate. To a stirred mixture of 6-(4-(5-(azetidin-3-ylmethyl)pyridin-2-yl)indolin-1-yl)-N-((1R,2S)-2-fluorocyclopropyl)-8-(methylamino)imidazo[1,2-b]pyridazine-3-carboxamide 2,2,2-trifluoroacetate (580 mg, 1.13 mmol, Intermediate QT) and tert-butyl 3,3-difluoro-4-oxopiperidine-1-carboxylate (266 mg, 1.13 mmol) in DMSO (12 mL) was added Et₃N (229 mg, 2.26 mmol) and HOAc (679 mg, 11.3 mmol) at rt. The resulting mixture was stirred for 1 h at 50° C. To the above mixture was added NaBH₃CN (356 mg, 5.660 mmol) in portions at rt. The resulting mixture was stirred for an additional 2 h at 50° C. On completion, the mixture was cooled to rt and purified directly by reversed-phase flash chromatography (Column: Spherical C18, 20˜40 μm, 330 g; Mobile Phase A: Water (plus 10 mM NH₄HCO₃), Mobile Phase B: ACN; Flow rate: 80 mL/min; Gradient (B %): 5%˜40%, 4 min; 40%˜60%, 8 min; 60%˜95%; 20 min; 95%, 5 min; Detector: 254 nm; the fractions containing desired product were collected at 81% B and concentrated under reduced pressure) to afford the title compound (650 mg, 78% yield) as a brown solid. LC/MS (ESI, m/z): [(M+1)]⁺=732.4.

Step 2—6-(4-(5-((1-(3,3-Difluoropiperidin-4-yl)azetidin-3-yl)methyl)pyridin-2-yl)indolin-1-yl)-N-((1R,2S)-2-fluorocyclopropyl)-8-(methylamino)imidazo[1,2-b]pyridazine-3-carboxamide trifluoroacetate. A mixture of tert-butyl 3,3-difluoro-4-(3-((6-(1-(3-(((1R,2S)-2-fluorocyclopropyl)carbamoyl)-8-(methylamino)imidazo[1,2-b]pyridazin-6-yl)indolin-4-yl)pyridin-3-yl)methyl)azetidin-1-yl)piperidine-1-carboxylate (600 mg, 0.820 mmol) in DCM (6 mL) and TFA (2 mL) was stirred for 1 h at rt. On completion, the mixture was concentrated under reduced pressure. The residue was purified by trituration with Et₂O (10 mL) to afford the title compound (500 mg, TFA salt) as a brown solid. LC/MS (ESI, m/z): [(M+1)]⁺=632.3.

### 4-Chloro-3-(2,6-dioxopiperidin-3-yl)benzoic acid (Intermediate QV)

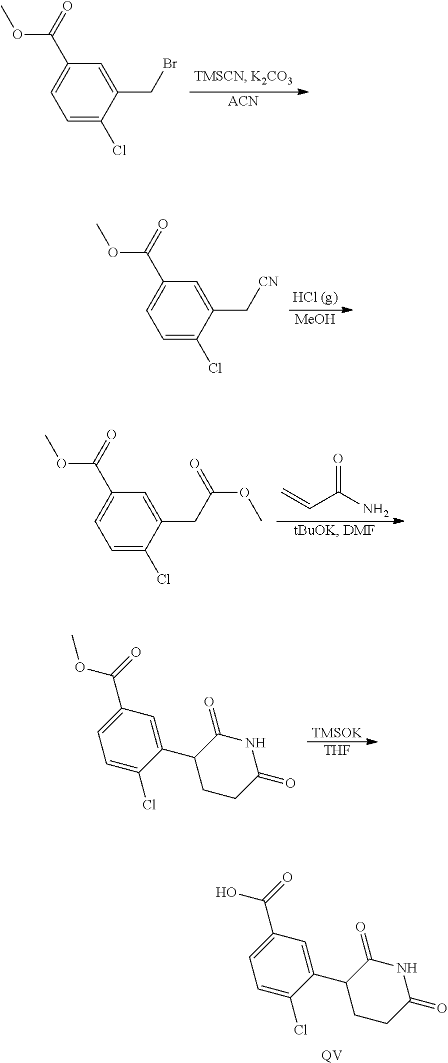

Step 1—Methyl 4-chloro-3-(cyanomethyl)benzoate. A mixture of methyl 3-(bromomethyl)-4-chlorobenzoate (5.00 g, 19.0 mmol), TMSCN (2.82 g, 28.5 mmol) and K₂CO₃ (3.93 g, 28.5 mmol) in ACN (250 mL) was stirred for overnight at 80° C. On completion, the mixture was cooled to rt and concentrated under reduced pressure. The resulting mixture was diluted with water (200 mL) and extracted with CH₂C2 (3×300 mL). The combined organic layers were washed with brine (1×500 mL), dried over anhydrous Na₂SO₄. After filtration, the filtrate was concentrated under reduced pressure to afford the title compound (3.70 g, 93% yield) as a white solid. LC/MS (ESI, m/z): [(M+1)]=210.0.

Step 2—Methyl 4-chloro-3-(2-methoxy-2-oxoethyl)benzoate. A mixture of methyl 4-chloro-3-(cyanomethyl)benzoate (3.70 g, 17.7 mmol) in MeOH (40 mL) was stirred for 2 h at 10° C. under HCl (g) atmosphere. Then the mixture was stirred for overnight at rt. On completion, the mixture was concentrated under reduced pressure. The resulting mixture was diluted with water (100 mL) and extracted with EtOAc (3×200 mL). The combined organic layers were washed with brine (3×300 mL), and dried over anhydrous Na₂SO₄. After filtration, the filtrate was concentrated under reduced pressure to afford the title compound (3.2 g) as a yellow oil.

Step 3—Methyl 4-chloro-3-(2,6-dioxopiperidin-3-yl)benzoate. To a stirred mixture of t-BuOK (693 mg, 6.18 mmol) in DMF (15 mL) were added methyl 4-chloro-3-(2-methoxy-2-oxoethyl)benzoate (1.00 g, 4.12 mmol) and acrylamide (292 mg, 4.12 mmol) in portions at rt under nitrogen atmosphere. The resulting mixture was stirred for 2 h at it under nitrogen atmosphere. On completion, the mixture was purified directly by reversed-phase flash chromatography (Column: Spherical C18, 20˜40 μm, 330 g; Mobile Phase A: Water (plus 10 mM NH₄HCO₃), Mobile Phase B: ACN; Flow rate: 80 mL/min; Gradient (B %): 5%˜40%, 4 min; 30%˜50%, 20 min; 50%˜95%; 2 min; 95%, 5 min; Detector: 254 nm; the fractions containing desired product were collected at 41% B and concentrated under reduced pressure) to afford the title compound (700 mg, 60% yield) as a white solid. LC/MS (ESI, m/z): [(M+1)]⁺=282.0.

Step 4—4-Chloro-3-(2,6-dioxopiperidin-3-yl)benzoic acid. A mixture of methyl 4-chloro-3-(2,6-dioxopiperidin-3-yl)benzoate (100 mg, 0.355 mmol) and potassium trimethylsilanolate (227 mg, 1.78 mmol) in THF (4 mL) was stirred for 1 h at rt. On completion, the mixture was purified directly by reversed-phase flash chromatography (Column: Spherical C18, 20˜40 μm, 120 g; Mobile Phase A: Water (plus 0.1% HCOOH), Mobile Phase B: ACN; Flow rate: 50 mL/min; Gradient (B %): 5%˜22%, 4 min; 22%˜50%, 20 min; 50%˜95%; 2 min; 95%, 5 min; Detector: 254 nm; the fractions containing desired product were collected at 40% B and concentrated under reduced pressure) to afford the title compound (85 mg, 89% yield) as a white solid. LC/MS (ESI, m/z): [(M+1)]⁺=268.0.

### 6-(4-(5-((3,9-Diazaspiro[5.5]undecan-3-yl)methyl)pyridin-2-yl)indolin-1-yl)-N-((1R,2S)-2-fluorocyclopropyl)-8-(methylamino)imidazo[1,2-b]pyridazine-3-carboxamide 2,2,2-trifluoroacetate (Intermediate QW)

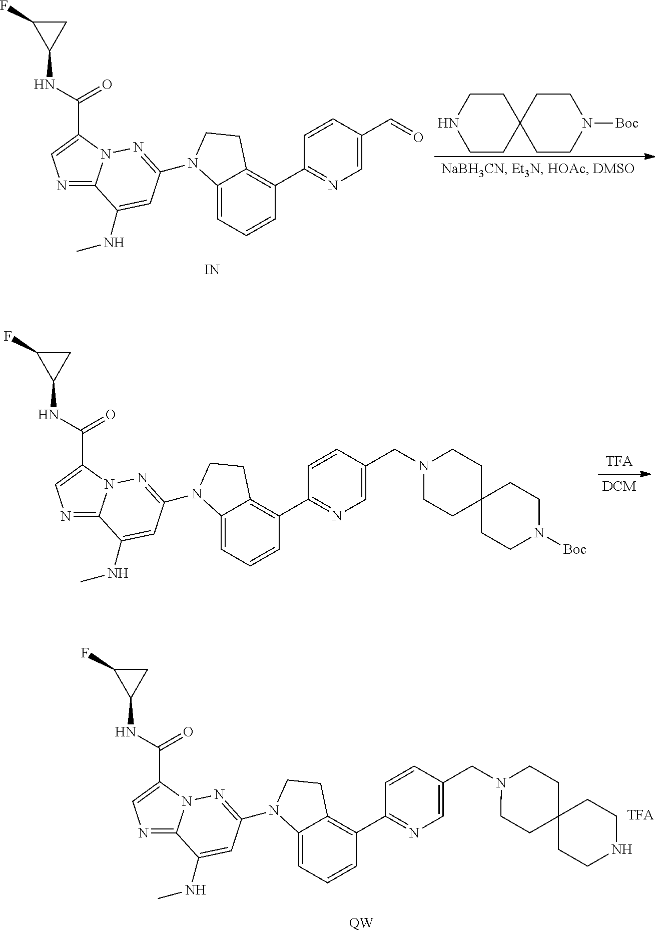

Step 1—Tert-butyl 9-((6-(1-(3-(((1R,2S)-2-fluorocyclopropyl)carbamoyl)-8-(methylamino)imidazo[1,2-b]pyridazin-6-yl)indolin-4-yl)pyridin-3-yl)methyl)-3,9-diazaspiro[5.5]undecane-3-carboxylate. To a stirred mixture of N-((1R,2S)-2-fluorocyclopropyl)-6-(4-(5-formylpyridin-2-yl)indolin-1-yl)-8-(methylamino)imidazo[1,2-b]pyridazine-3-carboxamide (300 mg, 0.636 mmol, Intermediate IN) and Et₃N (129 mg, 1.27 mmol) in DMSO (12 mL) were added tert-butyl 3,9-diazaspiro[5.5]undecane-3-carboxylate (162 mg, 0.636 mmol, CAS #173405-78-2) and HOAc (382 mg, 6.36 mmol) at rt. The resulting mixture was stirred for 1 h at rt. To the above mixture was added NaBH₃CN (200 mg, 3.18 mmol) in portions at rt. The resulting mixture was stirred for an additional 2 h at 50° C. On completion, the mixture was cooled to rt and purified directly by reversed-phase flash chromatography (Column: Spherical C18, 20-40 μm, 330 g; Mobile Phase A: Water (plus 10 mM NH₄HCO₃), Mobile Phase B: ACN; Flow rate: 80 mL/min; Gradient (B %): 5%˜40%, 4 min; 40%˜60%, 20 min; 60%˜95%; 2 min; 95%, 10 min; Detector: 254 nm; the fractions containing desired product were collected at 95% B and concentrated under reduced pressure) to afford the title compound (300 mg, 66% yield) as a brown solid. LC/MS (ESI, m/z): [(M+1)]=710.3.

Step 2—6-(4-(5-((3,9-Diazaspiro[5.5]undecan-3-yl)methyl)pyridin-2-yl)indolin-1-yl)-N-((1R,2S)-2-fluorocyclopropyl)-8-(methylamino)imidazo[1,2-b]pyridazine-3-carboxamide 2,2,2-trifluoroacetate. A mixture of tert-butyl 9-((6-(1-(3-(((1R,2S)-2-fluorocyclopropyl)carbamoyl)-8-(methylamino)imidazo[1,2-b]pyridazin-6-yl)indolin-4-yl)pyridin-3-yl)methyl)-3,9-diazaspiro[5.5]undecane-3-carboxylate (280 mg, 0.394 mmol) in TFA (1 mL) and DCM (3 mL) was stirred for 1 h at rt. On completion, the mixture was concentrated under reduced pressure. The residue was purified by trituration with Et₂O (10 mL) to afford the title compound (210 mg, TFA salt) as a brown solid. LC/MS (ESI, m/z): [(M+1)]=610.4.

### N-[(1R,2S)-2-fluorocyclopropyl]-6-[4-(5-formylpyridin-2-yl)-2,3-dihydroindol-1-yl]-8-(methylamino)imidazo[1,2-b]pyridazine-3-carboxamide (Intermediate QX)

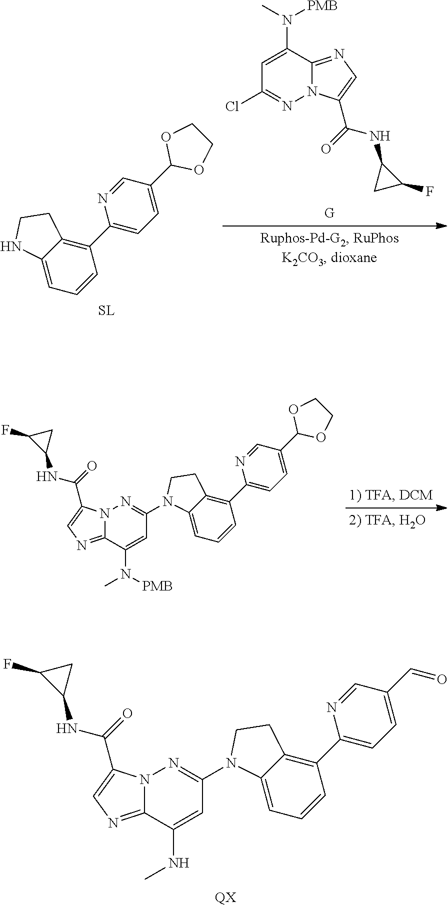

Step 1—6-(4-(5-(1,3-Dioxolan-2-yl)pyridin-2-yl)indolin-1-yl)-N-((1R,2S)-2-fluorocyclopropyl)-8-((4-methoxybenzyl)(methyl)amino)imidazo[1,2-b]pyridazine-3-carboxamide. To a stirred mixture of 4-[5-(1,3-dioxolan-2-yl)pyridin-2-yl]-2,3-dihydro-1H-indole (11.00 g, 41.00 mmol, Intermediate SL) and 6-chloro-N-[(1R,2S)-2-fluorocyclopropyl]-8-{[(4-methoxyphenyl)methyl](methyl)amino}imidazo[1,2-b]pyridazine-3-carboxamide (19.87 g, 49.20 mmol, Intermediate G) in dioxane (100 mL) were added RuPhos-PdOMs-2nd G (3.43 g, 4.10 mmol), RuPhos (3.83 g, 8.20 mmol) and K₂CO₃ (17.00 g, 123.0 mmol) in portions at rt under nitrogen atmosphere. The mixture was then stirred for 4 h at 100° C. under nitrogen atmosphere. On completion, the mixture was cooled to rt and purified by silica gel column chromatography, eluted with PE/EA (5:1˜1:10), to afford the title compound (15.00 g, 58% yield) as a yellow solid. LC/MS (ESI, m/z): [(M+H)]⁺=636.3.

Step 2—N-((1R,2S)-2-fluorocyclopropyl)-6-(4-(5-formylpyridin-2-yl)indolin-1-yl)-8-(methylamino)imidazo[1,2-b]pyridazine-3-carboxamide. A mixture of 6-{4-[5-(1,3-dioxolan-2-yl)pyridin-2-yl]-2,3-dihydroindol-1-yl}-N-[(1R,2S)-2-fluorocyclopropyl]-8-{[(4-methoxyphenyl)methyl](methyl)amino}imidazo[1,2-b]pyridazine-3-carboxamide (15.00 g, 23.60 mmol) and TFA (240 mL) in DCM (100 mL) was stirred for 2 h at rt under nitrogen atmosphere. Next, the mixture was concentrated under vacuum. Then H₂O (100 mL, 2 mol/L TFA) was added and stirred for 16 h at 50° C. under nitrogen atmosphere. On completion, the mixture was concentrated under vacuum. The resulting mixture was filtered and the filter cake was washed with diethyl ether (5×20 mL). The filtrate was concentrated under reduced pressure to give the title compound (8 g, 69% yield) as a yellow solid. ¹H NMR (400 MHz, DMSO-d₆) δ 10.18 (s, 1H), 9.21 (s, 1H), 8.89-8.84 (m, 1H), 8.39-8.32 (m, 1H), 8.05-7.99 (m, 1H), 7.97 (s, 1H), 7.85 (d, J=7.8 Hz, 1H), 7.75-7.67 (m, 1H), 7.46-7.39 (m, 1H), 7.39-7.31 (m, 1H), 6.03 (s, 1H), 4.91-4.70 (m, 1H), 4.23-4.14 (m, 2H), 3.54-3.46 (m, 2H), 3.09-3.01 (m, 1H), 2.98 (d, J=4.9 Hz, 3H), 1.30-1.16 (m, 1H), 1.05-0.90 (m, 1H). LC/MS (ESI, m/z): [(M+H)]⁺=472.3.

### 3-(3-Methyl-2-oxo-5-(4-(piperidin-4-yl)piperazin-1-yl)-2,3-dihydro-1H-benzo[d]imidazol-1-yl)piperidine-2,6-dione hydrochloride (Intermediate QY)

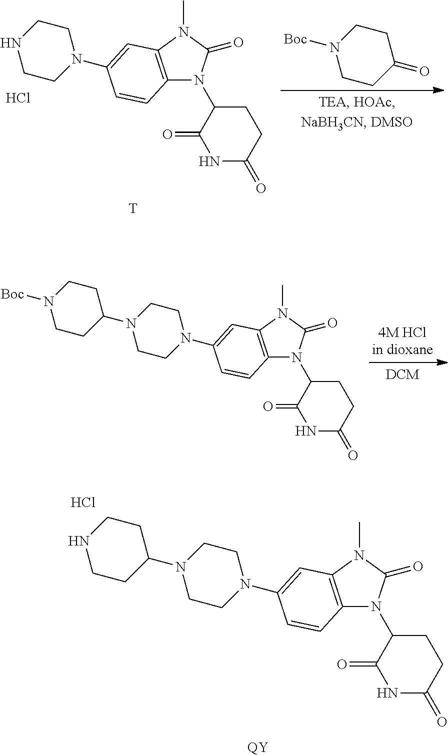

Step 1—Tert-butyl 4-(4-(1-(2,6-dioxopiperidin-3-yl)-3-methyl-2-oxo-2,3-dihydro-1H-benzo[d]imidazol-5-yl)piperazin-1-yl)piperidine-1-carboxylate. To a stirred solution of 3-(3-methyl-2-oxo-5-(piperazin-1-yl)-2,3-dihydro-1H-benzo[d]imidazol-1-yl)piperidine-2,6-dione hydrochloride (840 mg, 2.211 mmol, Intermediate T) in DMSO (10 mL) was added TEA (448 mg, 4.422 mmol) at rt under nitrogen atmosphere and the mixture was stirred for 5 min at rt. The mixture was then basified to pH 8 with additional TEA. To the above mixture was added tert-butyl 4-oxopiperidine-1-carboxylate (441 mg, 2.21 mmol, CAS #79099-07-3) and HOAc (398 mg, 6.63 mmol) at rt. The resulting mixture was stirred for an additional 30 min at rt. To the above mixture was added NaBH₃CN (417 mg, 6.633 mmol) in portions over 5 min at rt. The resulting mixture was stirred for 2 h at 50° C. under nitrogen atmosphere. After completion of the reaction, the reaction mixture was cooled to rt and purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 20%-40% B in 35 min; Flow rate: 80 mL/min; Detector: 220/254 nm; desired fractions were collected at 38% B and concentrated under reduced pressure) to afford the title compound (700 mg, 60% yield) as a brown oil. LC/MS (ESI, m/z): [(M+H)]⁺=527.3.

Step 2—3-(3-Methyl-2-oxo-5-(4-(piperidin-4-yl)piperazin-1-yl)-2,3-dihydro-1H-benzo[d]imidazol-1-yl)piperidine-2,6-dione hydrochloride. To a solution of tert-butyl 4-(4-(1-(2,6-dioxopiperidin-3-yl)-3-methyl-2-oxo-2,3-dihydro-1H-benzo[d]imidazol-5-yl)piperazin-1-yl)piperidine-1-carboxylate (700 mg, 1.33 mmol) in DCM (10 mL) was added 4 M HCl (gas) in 1,4-dioxane (5 mL) dropwise at rt. The reaction mixture was stirred for 2 h at rt under nitrogen atmosphere. On completion, the mixture was concentrated under reduced pressure. The residue was purified by trituration with Et₂O (30 mL) to afford the title compound (500 mg, 81% yield) as an off-white solid. LC/MS (ESI, m/z): [(M+H)]⁺=427.2.

### 3-(5-(4-(3′,3′-Difluoro-[1,4′-bipiperidin]-4-yl)piperazin-1-yl)-3-methyl-2-oxo-2,3-dihydro-1H-benzo[d]imidazol-1-yl)piperidine-2,6-dione trifluoroacetate (Intermediate QZ)

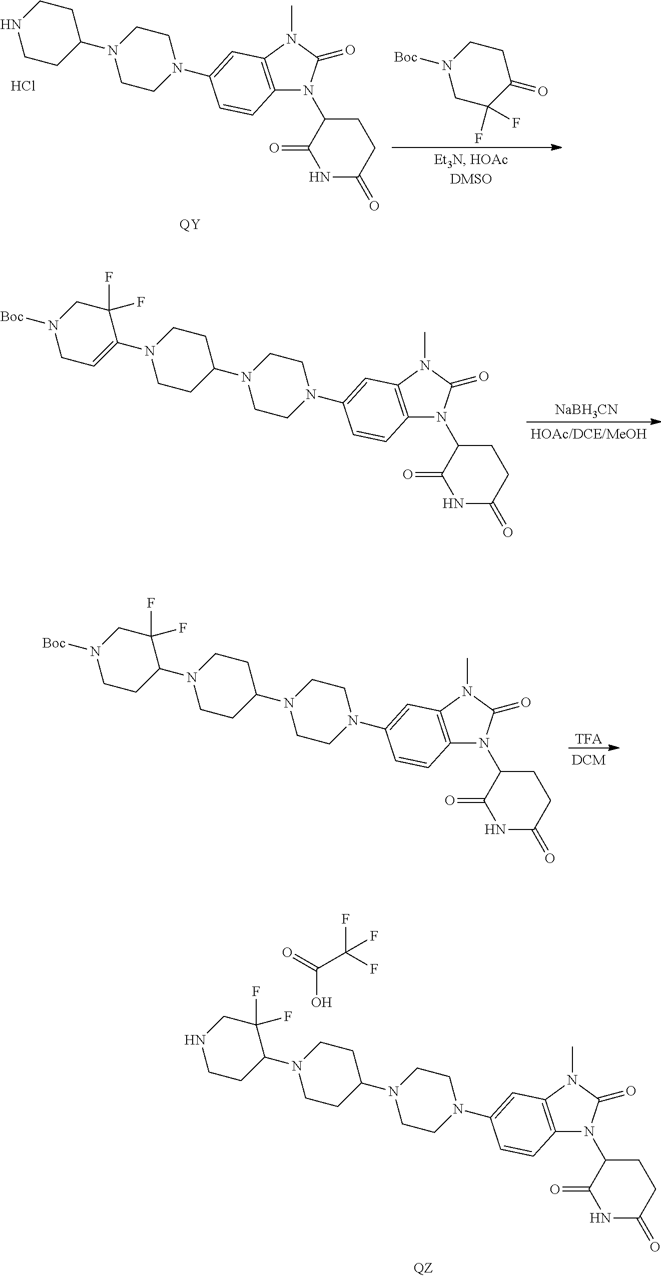

Step 1—Tert-butyl 4-(4-(4-(1-(2,6-dioxopiperidin-3-yl)-3-methyl-2-oxo-2,3-dihydro-1H-benzo[d]imidazol-5-yl)piperazin-1-yl)piperidin-1-yl)-3,3-difluoro-3,6-dihydropyridine-1(2H)-carboxylate. To a stirred solution of 3-(3-methyl-2-oxo-5-(4-(piperidin-4-yl)piperazin-1-yl)-2,3-dihydro-1H-benzo[d]imidazol-1-yl)piperidine-2,6-dione hydrochloride (400 mg, 0.864 mmol, Intermediate QY) in DMSO (10 mL) was added TEA (131 mg, 1.30 mmol) at rt under nitrogen atmosphere. To the above mixture was added tert-butyl 3,3-difluoro-4-oxopiperidine-1-carboxylate (305 mg, 1.30 mmol) and HOAc (156 mg, 2.592 mmol) at rt. The resulting mixture was stirred for 2 h at 100° C. On completion, the mixture was cooled to rt and diluted with water (200 mL). The resulting mixture was extracted with EtOAc (2×200 mL). The combined organic layers were washed with brine (400 mL), dried over anhydrous Na₂SO₄. After filtration, the filtrate was concentrated under reduced pressure to give the title compound (1.00 g) as a yellow oil. LC/MS (ESI, m/z): [(M+H)]⁺=644.4.

Step 2—Tert-butyl 4-(4-(1-(2,6-dioxopiperidin-3-yl)-3-methyl-2-oxo-2,3-dihydro-1H-benzo[d]imidazol-5-yl)piperazin-1-yl)-3′,3′-difluoro-[1,4′-bipiperidine]-1′-carboxylate. To a stirred solution of tert-butyl 4-(4-(4-(1-(2,6-dioxopiperidin-3-yl)-3-methyl-2-oxo-2,3-dihydro-1H-benzo[d]imidazol-5-yl)piperazin-1-yl)piperidin-1-yl)-3,3-difluoro-3,6-dihydropyridine-1(2H)-carboxylate (560 mg, 0.870 mmol) in DCE (10 mL) and MeOH (10 mL) were added HOAc (1 mL) and NaBH₃CN (273 mg, 4.35 mmol) at rt and the mixture was stirred for overnight at rt under nitrogen atmosphere. On completion, the mixture was concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 20%-40% B in 25 min; Flow rate: 80 mL/min; Detector: 220/254 nm; desired fractions were collected at 38% B and concentrated under reduced pressure) to afford the title compound (160 mg, 28% yield) as a white solid. LC/MS (ESI, m/z): [(M+H)]⁺=646.3.

Step 3—3-(5-(4-(3′,3′-Difluoro-[1,4′-bipiperidin]-4-yl)piperazin-1-yl)-3-methyl-2-oxo-2,3-dihydro-1H-benzo[d]imidazol-1-yl)piperidine-2,6-dione trifluoroacetate. To a solution of tert-butyl 4-(4-(1-(2,6-dioxopiperidin-3-yl)-3-methyl-2-oxo-2,3-dihydro-1H-benzo[d]imidazol-5-yl)piperazin-1-yl)-3′,3′-difluoro-[1,4′-bipiperidine]-1-carboxylate (160 mg, 0.248 mmol) in DCM (3 mL) was added TFA (1 mL) dropwise at rt and the mixture was stirred for 1 h at rt under nitrogen atmosphere. On completion, the mixture was concentrated under reduced pressure. The residue was purified by trituration with Et₂O (30 mL) to afford the title compound (170 mg, 85% yield) as a light yellow solid. LC/MS (ESI, m/z): [(M+H)]⁺=546.3.

### Tert-butyl 3,3-difluoro-4-(6-oxo-2-azaspiro[3.3]heptan-2-yl)piperidine-1-carboxylate (Intermediate RA)

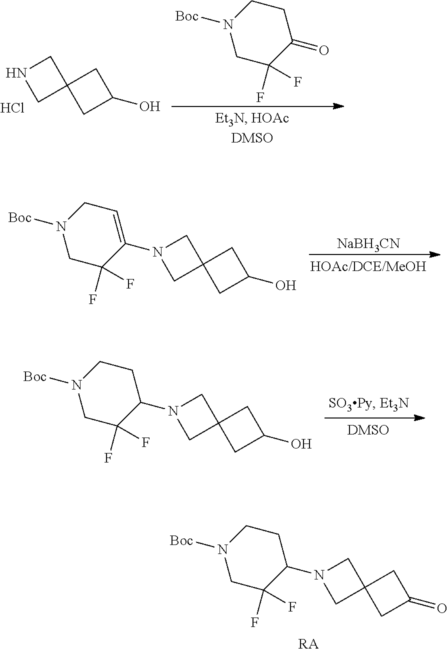

Step 1—Tert-butyl 3,3-difluoro-4-(6-hydroxy-2-azaspiro[3.3]heptan-2-yl)-3,6-dihydropyridine-1(2H)-carboxylate. To a stirred solution of 2-azaspiro[3.3]heptan-6-ol hydrochloride ( ) (2.00 g, 13.4 mmol, CAS #1630907-10-6) in DMSO (20 mL) was added TEA (2.03 g, 20.1 mmol) at rt under nitrogen atmosphere. To the above mixture was added tert-butyl 3,3-difluoro-4-oxopiperidine-1-carboxylate (4.72 g, 20.1 mmol, CAS #1215071-17-2) and HOAc (2.41 g, 40.10 mmol) at rt. The resulting mixture was stirred for 2 h at 100° C. On completion, the mixture was cooled to rt and diluted with water (200 mL). The resulting mixture was extracted with EtOAc (2×300 mL). The combined organic layers were washed with brine (400 mL), and dried over anhydrous Na₂SO₄. After filtration, the filtrate was concentrated under reduced pressure to give the title compound (4.00 g, 45% yield) as a brown oil. LC/MS (ESI, m/z): [(M+H)]⁺=331.3.

Step 2—Tert-butyl 3,3-difluoro-4-(6-hydroxy-2-azaspiro[3.3]heptan-2-yl)piperidine-1-carboxylate To a stirred solution of tert-butyl 3,3-difluoro-4-(6-hydroxy-2-azaspiro[3.3]heptan-2-yl)-3,6-dihydropyridine-1(2H)-carboxylate (4.00 g, 12.1 mmol) in DCE (10 mL) and MeOH (10 mL) were added HOAc (1 mL) and NaBH₃CN (3.80 g, 60.5 mmol) at rt and the mixture was stirred for overnight at rt under nitrogen atmosphere. On completion, the mixture was concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 20%-40% B in 25 min; Flow rate: 80 mL/min; Detector: 200/254 nm; desired fractions were collected at 38% B and concentrated under reduced pressure) to afford the title compound (1.00 g, 25% yield) as a yellow solid. LC/MS (ESI, m/z): [(M+H)]⁺=333.3.

Step 3—Tert-butyl 3,3-difluoro-4-(6-oxo-2-azaspiro[3.3]heptan-2-yl)piperidine-1-carboxylate. To a stirred solution of tert-butyl 3,3-difluoro-4-(6-hydroxy-2-azaspiro[3.3]heptan-2-yl)piperidine-1-carboxylate (600 mg, 1.81 mmol) in DMSO (15 mL) was added TEA (1.46 g, 14.44 mmol) at 0° C. under nitrogen atmosphere. To the above mixture was added SO₃-pyridine (1.15 g, 7.22 mmol) dropwise at 0° C. The resulting mixture was stirred for 1 h at rt. The resulting mixture was used in the next step directly without further purification. LC/MS (ESI, m/z): [(M+H)]⁺=331.1.

### 3-(5-(4-(2-(3,3-Difluoropiperidin-4-yl)-2-azaspiro[3.3]heptan-6-yl)piperazin-1-yl)-3-methyl-2-oxo-2,3-dihydro-1H-benzo[d]imidazol-1-yl)piperidine-2,6-dione trifluoroacetate (Intermediate RB)

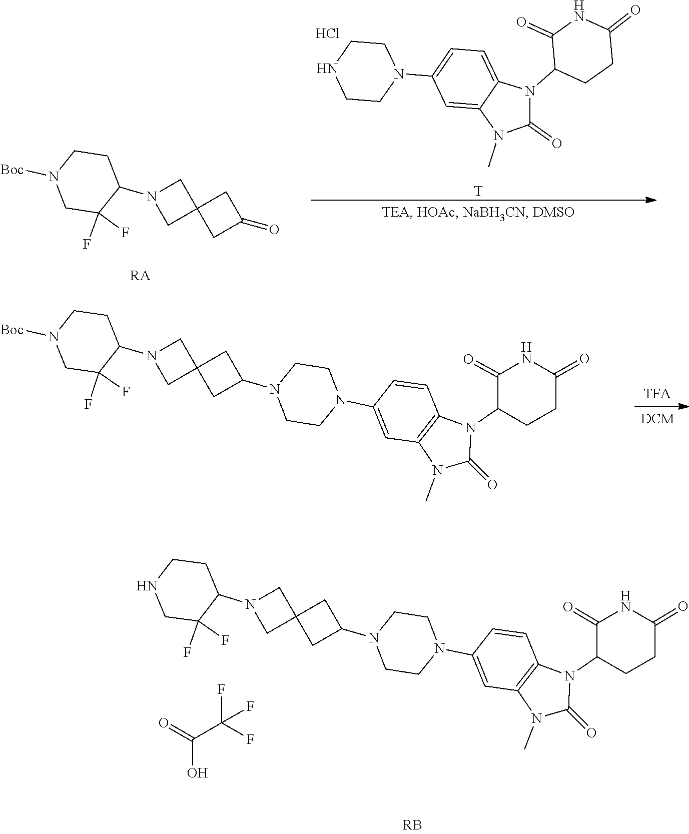

Step 1—Tert-butyl 4-(6-(4-(1-(2,6-dioxopiperidin-3-yl)-3-methyl-2-oxo-2,3-dihydro-1H-benzo[d]imidazol-5-yl)piperazin-1-yl)-2-azaspiro[3.3]heptan-2-yl)-3,3-difluoropiperidine-1-carboxylate. To a stirred solution of 3-(3-methyl-2-oxo-5-(piperazin-1-yl)-2,3-dihydro-1H-benzo[d]imidazol-1-yl)piperidine-2,6-dione hydrochloride (460 mg, 1.21 mmol, Intermediate T) in DMSO (10 mL) were added TEA (245 mg, 2.42 mmol) at rt under nitrogen atmosphere and the mixture was stirred for 5 min at rt. The mixture was basified to pH 8 with additional TEA. To the above mixture was added tert-butyl 3,3-difluoro-4-(6-oxo-2-azaspiro[3.3]heptan-2-yl)piperidine-1-carboxylate (400 mg, 1.21 mmol, Intermediate RA) and HOAc (218 mg, 3.63 mmol) at rt. The resulting mixture was stirred for an additional 30 min at rt. To the above mixture was added NaBH₃CN (228 mg, 3.633 mmol) in portions over 5 min at rt. The resulting mixture was stirred for 2 h at 50° C. under nitrogen atmosphere. After completion of the reaction, the reaction mixture was cooled to rt and purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 35%-60% B in 25 min; Flow rate: 80 mL/min; Detector: 220/254 nm; desired fractions were collected at 46% B and concentrated under reduced pressure) to afford the title compound (150 mg, 19% yield) as a yellow solid. LC/MS (ESI, m/z): [(M+H)]⁺=290.1.

Step 2—3-(5-(4-(2-(3,3-Difluoropiperidin-4-yl)-2-azaspiro[3.3]heptan-6-yl)piperazin-1-yl)-3-methyl-2-oxo-2,3-dihydro-1H-benzo[d]imidazol-1-yl)piperidine-2,6-dione trifluoroacetate. To a stirred solution of tert-butyl 4-(6-(4-(1-(2,6-dioxopiperidin-3-yl)-3-methyl-2-oxo-2,3-dihydro-1H-benzo[d]imidazol-5-yl)piperazin-1-yl)-2-azaspiro[3.3]heptan-2-yl)-3,3-difluoropiperidine-1-carboxylate (150 mg, 0.228 mmol) in DCM (12 mL) was added TFA (4 mL) at rt under nitrogen atmosphere and the mixture was stirred for 2 h at rt under nitrogen atmosphere. On completion, the mixture was concentrated under reduced pressure. The residue was purified by trituration with Et₂O (30 mL) to give the title compound (200 mg, 94% yield) as a colorless oil. LC/MS (ESI, m/z): [(M+H)]⁺=558.5.

### Tert-butyl (3-(((1R,2S)-2-fluorocyclopropyl)carbamoyl)-6-(4-(3-formylbicyclo[1.1.1]pentan-1-yl)indolin-1-yl)imidazo[1,2-b]pyridazin-8-yl)(methyl)carbamate (Intermediate RC)

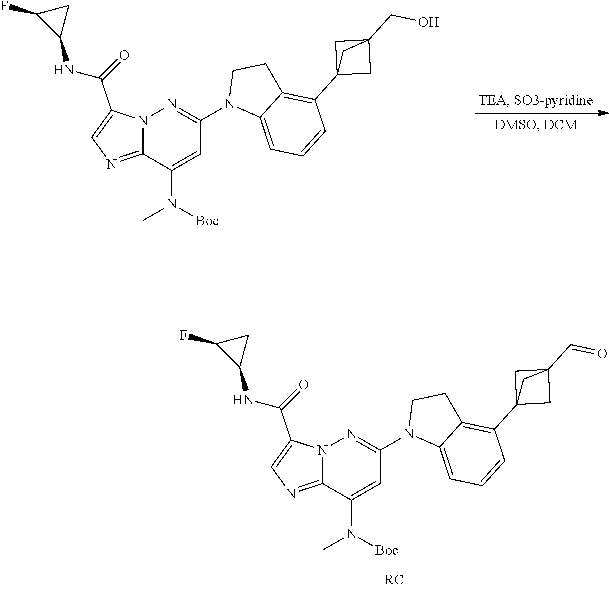 [SMILES: CN(C(=O)OC(C)(C)C)c1cc(N2CCc3c2cccc3C23CC(CO)(C2)C3)nn2c(C(=O)N[C@@H]3C[C@@H]3F)cnc12 ; CN(C(=O)OC(C)(C)C)c1cc(N2CCc3c2cccc3C23CC(C=O)(C2)C3)nn2c(C(=O)N[C@@H]3C[C@@H]3F)cnc12]

To a stirred solution of tert-butyl N-(3-{[(1R,2S)-2-fluorocyclopropyl]carbamoyl}-6-{₄-[3-(hydroxymethyl)bicyclo[1.1.1]pentan-1-yl]-2,3-dihydroindol-1-yl}imidazo[1,2-b]pyridazin-8-yl)-N-methylcarbamate (100 mg, 0.178 mmol, synthesized via Step 1 of Intermediate TP) in DMSO (3 mL) and DCM (1 mL) was added TEA (0.2 mL, 1.424 mmol) at 0° C. under nitrogen atmosphere. To the above mixture was added SO₃-pyridine (113.15 mg, 0.712 mmol) at 0° C. The resulting mixture was stirred for 1 h at rt. The solution was used in the next step directly without further purification. LC/MS (ESI, m/z): [(M+H)]⁺=561.3.

### 3-(4-(4-(Azetidin-3-yl)piperazin-1-yl)-3-methyl-2-oxo-2,3-dihydro-1H-benzo[d]imidazol-1-yl)piperidine-2,6-dione trifluoroacetate (Intermediate RD)

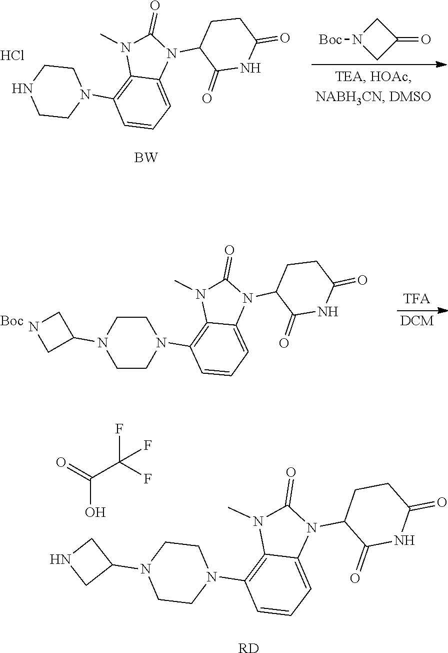

Step 1—Tert-butyl 3-(4-(1-(2,6-dioxopiperidin-3-yl)-3-methyl-2-oxo-2,3-dihydro-1H-benzo[d]imidazol-4-yl)piperazin-1-yl)azetidine-1-carboxylate. To a stirred solution of 3-(3-methyl-2-oxo-4-(piperazin-1-yl)-2,3-dihydro-1H-benzo[d]imidazol-1-yl)piperidine-2,6-dione hydrochloride (970 mg, 2.55 mmol, Intermediate BW) in DMSO (10 mL) were added TEA (517 mg, 5.11 mmol) at rt under nitrogen atmosphere and the mixture was stirred for 5 min at rt. To the above mixture was added tert-butyl 3-oxoazetidine-1-carboxylate (656 mg, 3.831 mmol, CAS #398489-26-4) and HOAc (460 mg, 7.662 mmol) at rt, followed by addition of NaBH₃CN (481 mg, 7.66 mmol) in portions over 5 min at rt. The resulting mixture was stirred for an additional 30 min at rt. Then the mixture was stirred for 2 h at 50° C. under nitrogen atmosphere. After completion of the reaction, the reaction mixture was cooled to rt and purified directly by reverse phase flash chromatography (Column: WelFlash™ C18-I, 20-40 μm, 120 g; Eluent A: Water (plus 10 mmol/L NH₄HCO₃); Eluent B: ACN; Gradient: 30%-50% B in 20 min; Flow rate: 80 mL/min; Detector: 220/254 nm; desired fractions were collected at 45% B and concentrated under reduced pressure) to afford the title compound (450 mg, 35% yield) as a brown solid. LC/MS (ESI, m/z): [(M+H)]⁺=499.2.

Step 2—3-(4-(4-(Azetidin-3-yl)piperazin-1-yl)-3-methyl-2-oxo-2,3-dihydro-1H-benzo[d]imidazol-1-yl)piperidine-2,6-dione trifluoroacetate. To a stirred solution of tert-butyl 3-(4-(1-(2,6-dioxopiperidin-3-yl)-3-methyl-2-oxo-2,3-dihydro-1H-benzo[d]imidazol-4-yl)piperazin-1-yl)azetidine-1-carboxylate (450 mg, 0.903 mmol) in DCM (21 mL) was added TFA (7 mL) at rt under nitrogen atmosphere and the mixture was stirred for 2 h at rt under nitrogen atmosphere. On completion, the mixture was concentrated under reduced pressure. The residue was purified by trituration with Et₂O (60 mL) to afford the title compound (630 mg, 98% yield) as a yellow solid. LC/MS (ESI, m/z): [(M+H)]⁺=399.2.

### 3-(4-(4-(1-(3,3-difluoropiperidin-4-yl)azetidin-3-yl)piperazin-1-yl)-3-methyl-2-oxo-2,3-dihydro-1H-benzo[d]imidazol-1-yl)piperidine-2,6-dione trifluoroacetate (Intermediate RE)

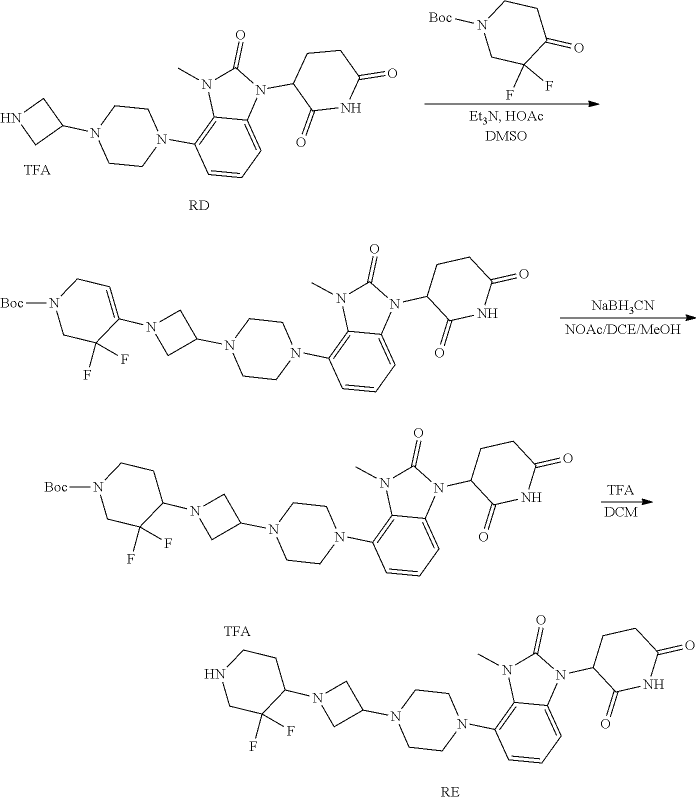

Step 1—Tert-butyl 4-(3-(4-(1-(2,6-dioxopiperidin-3-yl)-3-methyl-2-oxo-2,3-dihydro-1H-benzo[d]imidazol-4-yl)piperazin-1-yl)azetidin-1-yl)-3,3-difluoro-3,6-dihydropyridine-1(2H)-carboxylate. To a stirred solution of 3-(4-(4-(azetidin-3-yl)piperazin-1-yl)-3-methyl-2-oxo-2,3-dihydro-1H-benzo[d]imidazol-1-yl)piperidine-2,6-dione trifluoroacetate (630 mg, 1.27 mmol, Intermediate RD) in DMSO (10 mL) was added TEA (193 mg, 1.903 mmol) at rt under nitrogen atmosphere and mixture was stirred for 5 min at rt. To the above mixture was added tert-butyl 3,3-difluoro-4-oxopiperidine-1-carboxylate (448 mg, 1.903 mmol, CAS #1215071-17-2) and HOAc (229 mg, 3.807 mmol) at rt. Then the resulting mixture was stirred for 16 h at 100° C. under nitrogen atmosphere. On completion, the mixture was cooled to rt and diluted with water (200 mL). The resulting mixture was extracted with EtOAc (2×300 mL). The combined organic layers were washed with brine (400 mL), dried over anhydrous Na₂SO₄. After filtration, the filtrate was concentrated under reduced pressure to afford the title compound (1.00 g, 90% yield) as a yellow oil. LC/MS (ESI, m/z): [(M+H)]⁺=616.3.

Step 2—Tert-butyl 4-(3-(4-(1-(2,6-dioxopiperidin-3-yl)-3-methyl-2-oxo-2,3-dihydro-1H-benzo[d]imidazol-4-yl)piperazin-1-yl)azetidin-1-yl)-3,3-difluoropiperidine-1-carboxylate. To a stirred solution of tert-butyl 4-(3-(4-(1-(2,6-dioxopiperidin-3-yl)-3-methyl-2-oxo-2,3-dihydro-1H-benzo[d]imidazol-4-yl)piperazin-1-yl)azetidin-1-yl)-3,3-difluoro-3,6-dihydropyridine-1(2H)-carboxylate (970 mg, 1.575 mmol) in DCE (10 mL) and MeOH (10 mL) were added HOAc (1 mL) and NaBH₃CN (495 mg, 7.875 mmol) at rt. The resulting mixture was stirred for overnight at rt under nitrogen atmosphere. On completion, the mixture was concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 20%-40% B in 25 min; Flow rate: 80 mL/min; Detector: 220/254 nm; desired fractions were collected at 38% B and concentrated under reduced pressure) to afford the title compound (190 mg, 20% yield) as a yellow solid. LC/MS (ESI, m/z): [(M+H)]⁺=618.5.

Step 3—3-(4-(4-(1-(3,3-Difluoropiperidin-4-yl)azetidin-3-yl)piperazin-1-yl)-3-methyl-2-oxo-2,3-dihydro-TH-benzo[d]imidazol-1-yl)piperidine-2,6-dione trifluoroacetate. To a stirred solution of tert-butyl 4-(3-(4-(1-(2,6-dioxopiperidin-3-yl)-3-methyl-2-oxo-2,3-dihydro-1H-benzo[d]imidazol-4-yl)piperazin-1-yl)azetidin-1-yl)-3,3-difluoropiperidine-1-carboxylate (190 mg, 0.308 mmol) in DCM (9 mL) was added TFA (3 mL) at rt under nitrogen atmosphere. The resulting mixture was stirred for 2 h at rt under nitrogen atmosphere. On completion, the mixture was concentrated under reduced pressure. The residue was purified by trituration with Et₂O (30 mL) and dried to afford the title compound (160 mg, 85% yield) as an off-white solid. LC/MS (ESI, m/z): [(M+H)]⁺=518.2.

### Benzyl 4-(azetidin-3-yl)piperazine-1-carboxylate (Intermediate RF)

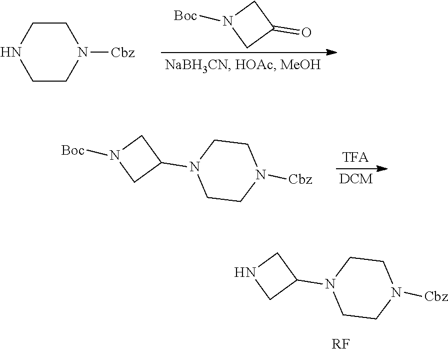

Step 1—Benzyl 4-[1-(tert-butoxycarbonyl)azetidin-3-yl]piperazine-1-carboxylate. To a stirred mixture of benzyl piperazine-1-carboxylate (70 g, 320 mmol) and tert-butyl 3-oxoazetidine-1-carboxylate (108.81 g, 635.58 mmol) in MeOH (1 L) was added HOAc (91.05 mL, 1589 mmol) at rt under nitrogen atmosphere. The resulting mixture was stirred for 30 min at 50° C. To the above mixture was added NaBH₃CN (59.91 g, 953.4 mmol) over 10 min at rt. The resulting mixture was stirred for an additional 2 h at 50° C. On completion, the mixture was cooled to rt and concentrated under reduced pressure. The reaction was quenched by the addition of water (300 mL) at rt and extracted with EtOAc (3×500 mL). The combined organic layers were washed with brine (1×500 mL), dried over anhydrous Na₂SO₄, filtered, and the filtrate was concentrated under reduced pressure. The residue was purified by silica gel column chromatography, eluted with PE/EA (1:1), to afford the title compound (111 g, 93% yield) as a white oil. LC/MS (ESI, m/z): [(M+H)]⁺=376.1.

Step 2—Benzyl 4-(azetidin-3-yl)piperazine-1-carboxylate. To a stirred mixture of benzyl 4-[1-(tert-butoxycarbonyl)azetidin-3-yl]piperazine-1-carboxylate (111 g, 296 mmol) in DCM (700 mL) was added TFA (350 mL) at rt under nitrogen atmosphere and the mixture was stirred for 1 h at rt. On completion, the mixture was concentrated under reduced pressure. The residue was basified to pH 9 with NaOH (aq.). The resulting mixture was extracted with CH₂Cl₂/MeOH (10:1) (3×800 mL). The combined organic layers were washed with brine (1×500 mL) and dried over anhydrous Na₂SO₄. After filtration, the filtrate was concentrated under reduced pressure to give the title compound (70 g, 86% yield) as a white oil. LC/MS (ESI, m/z): [(M+H)]⁺=276.2.

### Tert-butyl 3,3-difluoro-4-[3-(piperazin-1-yl)azetidin-1-yl]piperidine-1-carboxylate (Intermediate RG)

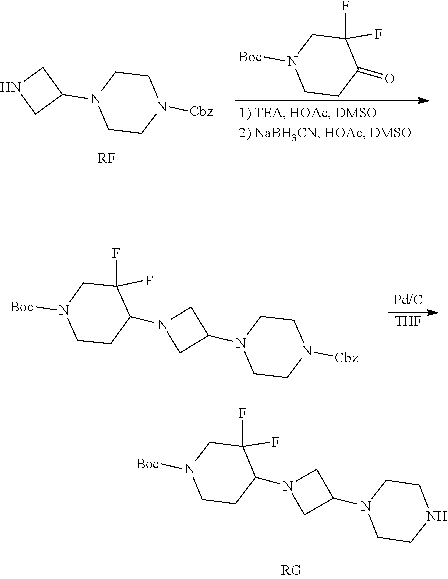

Step 1—Benzyl 4-{1-[1-(tert-butoxycarbonyl)-3,3-difluoropiperidin-4-yl]azetidin-3-yl}piperazine-1-carboxylate. To a stirred mixture of benzyl 4-(azetidin-3-yl)piperazine-1-carboxylate (20 g, 73 mmol, Intermediate RF) and tert-butyl 3,3-difluoro-4-oxopiperidine-1-carboxylate (17.09 g, 72.63 mmol) in DMSO (200 mL) were added TEA (30.29 mL, 217.9 mmol) and HOAc (20.81 mL, 363.2 mmol) at rt under nitrogen atmosphere. The resulting mixture was stirred for 2 h at 100° C. Then the mixture was allowed to cool to rt. To the above mixture were added MeOH (500 mL), HOAc (50 mL) and NaBH₃CN (44.65 g, 710.6 mmol) at rt. The resulting mixture was stirred for an additional 1 h at rt. On completion, the reaction was quenched by the addition of water (500 mL) at rt and the mixture was extracted with EtOAc (3×800 mL). The combined organic layers were washed with brine (3×800 mL), dried over anhydrous Na₂SO₄, filtered, and the filtrate was concentrated under reduced pressure. The residue was purified by silica gel column chromatography, eluted with CH₂Cl₂/EA (1:10), to afford the title compound (37 g, 53% yield) as a white oil. LC/MS (ESI, m/z): [(M+H)]⁺=495.2.

Step 2—Tert-butyl 3,3-difluoro-4-[3-(piperazin-1-yl)azetidin-1-yl]piperidine-1-carboxylate. To a stirred mixture of benzyl 4-{1-[1-(tert-butoxycarbonyl)-3,3-difluoropiperidin-4-yl]azetidin-3-yl}piperazine-1-carboxylate (37 g, 75 mmol) in THF (500 mL) was added Pd/C (23.88 g, 224.4 mmol) at rt under nitrogen atmosphere and the mixture was stirred for 2 h at rt. On completion, the mixture was filtered, the filter cake was washed with THF (3×300 mL). The filtrate was concentrated under reduced pressure to give the title compound (27.6 g) as a black oil. LC/MS (ESI, m/z): [(M+H)]⁺=361.2.

### 3-(4-{4-[1-(3,3-Difluoropiperidin-4-yl)azetidin-3-yl]piperazin-1-yl}-3-methyl-2-oxo-1,3-benzodiazol-1-yl)piperidine-2,6-dione trifluoroacetate (Intermediate RH)

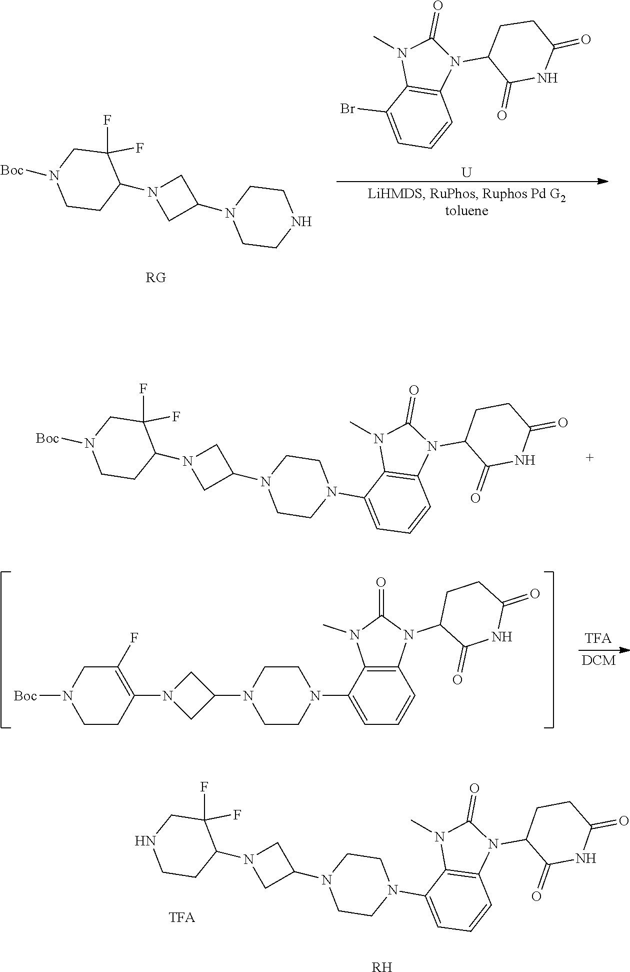

Step 1—Tert-butyl 4-(3-{4-[1-(2,6-dioxopiperidin-3-yl)-3-methyl-2-oxo-1,3-benzodiazol-4-yl]piperazin-1-yl}azetidin-1-yl)-3,3-difluoropiperidine-1-carboxylate and tert-butyl 4-(3-{4-[1-(2,6-dioxopiperidin-3-yl)-3-methyl-2-oxo-1,3-benzodiazol-4-yl]piperazin-1-yl}azetidin-1-yl)-3-fluoro-5,6-dihydro-2H-pyridine-1-carboxylate. To a stirred mixture of tert-butyl 3,3-difluoro-4-[3-(piperazin-1-yl)azetidin-1-yl]piperidine-1-carboxylate (27.6 g, 76.6 mmol, Intermediate RG) and 3-(4-bromo-3-methyl-2-oxo-1,3-benzodiazol-1-yl)piperidine-2,6-dione (21.58 g, 63.81 mmol, Intermediate U) in toluene (550 mL) were added RuPhos (2.98 g, 6.38 mmol), RuPhos-PdCl-2nd G (4.96 g, 6.38 mmol) and LiHMDS (505 mL, 510 mmol) at rt under nitrogen atmosphere. The resulting mixture was stirred for 1 h at 80° C. On completion, the mixture was cooled to rt and acidified to pH 5 with FA. The resulting mixture was concentrated under reduced pressure. The residue was purified by silica gel column chromatography, eluted with CH₂Cl₂/EA (1:10), to afford tert-butyl 4-(3-{4-[1-(2,6-dioxopiperidin-3-yl)-3-methyl-2-oxo-1,3-benzodiazol-4-yl]piperazin-1-yl}azetidin-1-yl)-3,3-difluoropiperidine-1-carboxylate (8.3 g, 21% yield) as a yellow solid and tert-butyl 4-(3-{4-[1-(2,6-dioxopiperidin-3-yl)-3-methyl-2-oxo-1,3-benzodiazol-4-yl]piperazin-1-yl}azetidin-1-yl)-3-fluoro-5,6-dihydro-2H-pyridine-1-carboxylate (6 g, 13% yield, byproduct) as a yellow solid. LC/MS (ESI, m/z): [(M+H)]⁺=618.3.

Step 2—3-(4-{4-[1-(3,3-Difluoropiperidin-4-yl)azetidin-3-yl]piperazin-1-yl}-3-methyl-2-oxo-1,3-benzodiazol-1-yl)piperidine-2,6-dione trifluoroacetate. To a stirred mixture of tert-butyl 4-(3-{4-[l-(2,6-dioxopiperidin-3-yl)-3-methyl-2-oxo-1,3-benzodiazol-4-yl]piperazin-1-yl}azetidin-1-yl)-3,3-difluoropiperidine-1-carboxylate (12.1 g, 19.6 mmol) in DCM (120 mL) was added TFA (60 mL) at rt under nitrogen atmosphere and the mixture was stirred for 1 h at it. On completion, the mixture was concentrated under reduced pressure. The residue was purified by trituration with Et₂O (100 mL) to give the title compound (12.2 g, 99% yield) as a yellow solid. LC/MS (ESI, m/z): [(M+H)]⁺=518.3.

### 3-(2,4-Dioxo-1,3-diazinan-1-yl)-4-methylbenzoic acid (Intermediate RI)

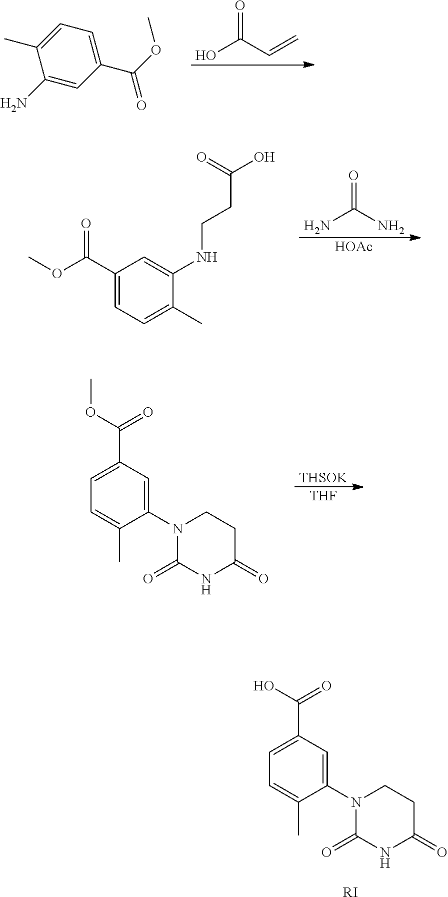

Step 1—3-{[5-(Methoxycarbonyl)-2-methylphenyl]amino}propanoic acid. To a stirred solution of methyl 3-amino-4-methylbenzoate (20 g, 120 mmol) in acrylic acid (17.45 g, 242.1 mmol) at rt under nitrogen atmosphere. The resulting mixture was stirred for 16 h at 100° C. under nitrogen atmosphere. On completion, the reaction was cooled to at and quenched with water (200 mL). The resulting mixture was extracted with EtOAc (3×500 mL). The combined organic layers were washed with brine (2×500 mL), and dried over anhydrous Na₂SO₄. After filtration, the filtrate was concentrated under reduced pressure to afford the title compound (30 g) as a brown solid. LC/MS (ESI, m/z): [(M+H)]^{T}=238.1.

Step 2—Methyl 3-(2,4-dioxo-1,3-diazinan-1-yl)-4-methylbenzoate. To a stirred solution of 3-{[5-(methoxycarbonyl)-2-methylphenyl]amino}propanoic acid (30 g, 120 mmol) in HOAc (400 mL) was added urea (53.16 g, 885.1 mmol) at rt under nitrogen atmosphere. The resulting mixture was stirred for 16 h at 110° C. under nitrogen atmosphere. On completion, the reaction was cooled to rt and quenched with water (300 mL) at rt. The precipitated solids were collected by filtration and washed with water (3×5 mL) to afford the title compound (25 g, 75% yield) as a white solid. LC/MS (ESI, m/z): [(M+H)]f=263.1.

Step 3—3-(2,4-Dioxo-1,3-diazinan-1-yl)-4-methylbenzoic acid. To a stirred mixture of methyl 3-(2,4-dioxo-1,3-diazinan-1-yl)-4-methylbenzoate (2.00 g, 7.63 mmol) in THF (50 mL) was added potassium trimethylsilanolate (4.89 g, 38.1 mmol) at rt under nitrogen atmosphere. The resulting mixture was stirred for 2 h at rt under nitrogen atmosphere. On completion, the mixture was acidified to pH 3 with HCl (2M). The precipitated solids were collected by filtration and washed with water (3×5 mL) to give the title compound (1.00 g, 53% yield) as a white solid. LC/MS (ESI, m/z): [(M+H)]⁺=249.1. Benzyl 4-(piperidin-4-ylmethyl)-2,3-dihydroindole-1-carboxylate trifluoroacetate (Intermediate RJ)

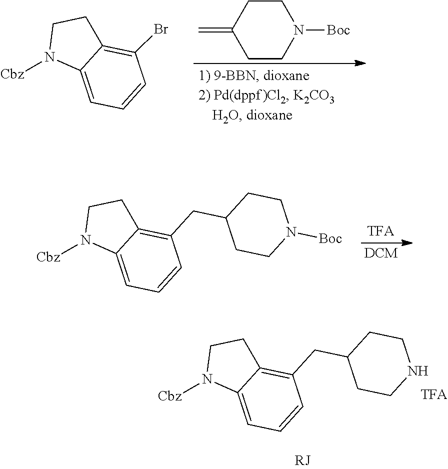

Step 1—Tert-butyl 4-{4-[4-chloro-3-(2,4-dioxo-1,3-diazinan-1-yl)benzoyl]piperazin-1-yl}piperidine-1-carboxylate. To a stirred mixture of tert-butyl 4-methylidenepiperidine-1-carboxylate (2.97 g, 15.1 mmol) and 9-borabicyclo[3.3.1]nonane (2.39 g, 19.6 mmol) in dioxane (60 mL) was stirred for 1 h at 50° C. under nitrogen atmosphere. Then the mixture was allowed to cool tort. To the above mixture were added benzyl 4-bromo-2,3-dihydroindole-1-carboxylate (5 g, 15 mmol, synthesized via Step 1 of Intermediate OK), K₂CO₃ (6.24 g, 45.2 mmol), Pd(dppf)Cl₂CH₂Cl₂ (1.23 g, 1.51 mmol) and H₂O (12 mL) at rt under nitrogen atmosphere. The resulting mixture was stirred for 2 h at 80° C. under nitrogen atmosphere. On completion, the mixture was cooled to rt and concentrated under reduced pressure. The residue was purified by silica gel column chromatography, eluted with PE/EA (10:1˜3:1), to afford the title compound (5.2 g, 77% yield) as a yellow oil. LC/MS (ESI, m/z): [(M+H)]⁺=451.1.

Step 2—Benzyl 4-(piperidin-4-ylmethyl)-2,3-dihydroindole-1-carboxylate trifluoroacetate. To a stirred solution of benzyl 4-{[1-(tert-butoxycarbonyl)piperidin-4-yl]methyl}-2,3-dihydroindole-1-carboxylate (5.2 g, 12 mmol) in DCM (50 mL) was added TFA (25 mL) at rt and the mixture was stirred for 1 h at rt. On completion, the mixture was concentrated under reduced pressure. The residue was purified by trituration with Et₂O (2×50 mL) to afford the title compound (4.2 g) as an off-white solid. LC/MS (ESI, m/z): [(M+H)]⁺=351.1.

### Benzyl 4-({3′,3′-difluoro-[1,4′-bipiperidin]-4-yl}methyl)-2,3-dihydroindole-1-carboxylate (Intermediate RK)

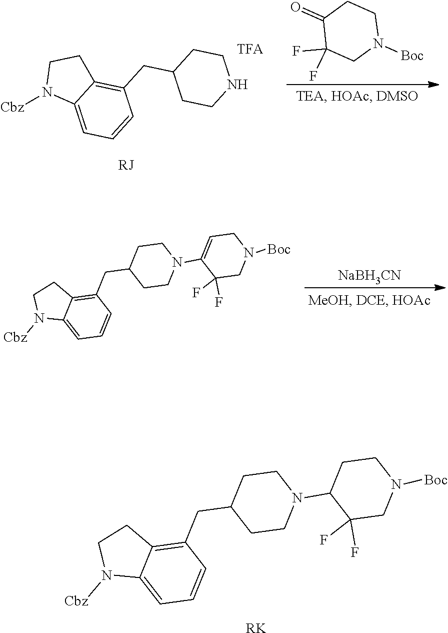 [SMILES: O=C(OCc1ccccc1)N1CCc2c(CC3CCNCC3)cccc21 ; CC(C)(C)OC(=O)N1CCC(=O)C(F)(F)C1 ; CC(C)(C)OC(=O)N1CC=C(N2CCC(Cc3cccc4c3CCN4C(=O)OCc3ccccc3)CC2)C(F)(F)C1 ; "NaBH3CN" ; CC(C)(C)OC(=O)N1CCC(N2CCC(Cc3cccc4c3CCN4C(=O)OCc3ccccc3)CC2)C(F)(F)C1 ; "RK"]

Step 1—Benzyl 4-({l-[1-(tert-butoxycarbonyl)-3,3-difluoro-2,6-dihydropyridin-4-yl]piperidin-4-yl}methyl)-2,3-dihydroindole-1-carboxylate. To a stirred mixture of benzyl 4-(piperidin-4-ylmethyl)-2,3-dihydroindole-1-carboxylate trifluoroacetate (1.91 g, 4.26 mmol, Intermediate RJ) and TEA (1.78 mL, 12.8 mmol) in DMSO (15 mL) were added tert-butyl 3,3-difluoro-4-oxopiperidine-1-carboxylate (2.00 g, 8.52 mmol) and HOAc (1.46 mL, 25.6 mmol) at rt. The resulting mixture was stirred for 2 h at 100° C. On completion, the mixture was cooled to rt and concentrated under reduced pressure. The residue was diluted with water (200 mL) and extracted with EtOAc (3×300 mL). The combined organic layers were washed with brine (1×300 mL), dried over anhydrous Na₂SO₄. After filtration, the filtrate was concentrated under reduced pressure to give the title compound (3.5 g) as a yellow oil. LC/MS (ESI, m/z): [(M+H)]⁺=568.2.

Step 2—Tert-butyl 4-({1-[(benzyloxy)carbonyl]-2,3-dihydroindol-4-yl}methyl)-3′,3′-difluoro-[1,4′-bipiperidine]-1′-carboxylate. To a stirred solution of benzyl 4-({1-[1-(tert-butoxycarbonyl)-3,3-difluoro-2,6-dihydropyridin-4-yl]piperidin-4-yl}methyl)-2,3-dihydroindole-1-carboxylate (3.5 g, 6.2 mmol) in DCE (20 mL), MeOH (20 mL) and HOAc (2 mL) at rt and the mixture was stirred for 2 h at rt. On completion, the mixture was concentrated under reduced pressure. The residue was purified by silica gel column chromatography, eluted with PE/EA (8:1˜3:1), to afford the title compound (2.5 g, 71% yield) as a yellow oil. LC/MS (ESI, m/z): [(M+H)]⁺=570.3.

Step 3—Benzyl 4-({3′,3′-difluoro-[1,4′-bipiperidin]-4-yl}methyl)-2,3-dihydroindole-1-carboxylate. To a stirred solution of tert-butyl 4-({1-[(benzyloxy)carbonyl]-2,3-dihydroindol-4-yl}methyl)-3′,3′-difluoro-[1,4′-bipiperidine]-1′-carboxylate (2.5 g, 4.4 mmol) in DCM (15 mL) was added TFA (10 mL) at rt and the mixture was stirred for 1 h at rt under nitrogen atmosphere. On completion, the mixture was concentrated under reduced pressure. The residue was basified to pH 8 with saturated Na₂CO₃ (aq.). The resulting mixture was extracted with EtOAc (3×150 mL). The combined organic layers were washed with brine (1×200 mL), dried over anhydrous Na₂SO₄. After filtration, the filtrate was concentrated under reduced pressure to give the title compound (2 g) as a yellow oil. LC/MS (ESI, m/z): [(M+H)]⁺=470.2.

### Tert-butyl 4-[4-(2,3-dihydro-1H-indol-4-ylmethyl)piperidin-1-yl]-3,3-difluoro-[1,4′-bipiperidine]-1′-carboxylate (Intermediate RL)

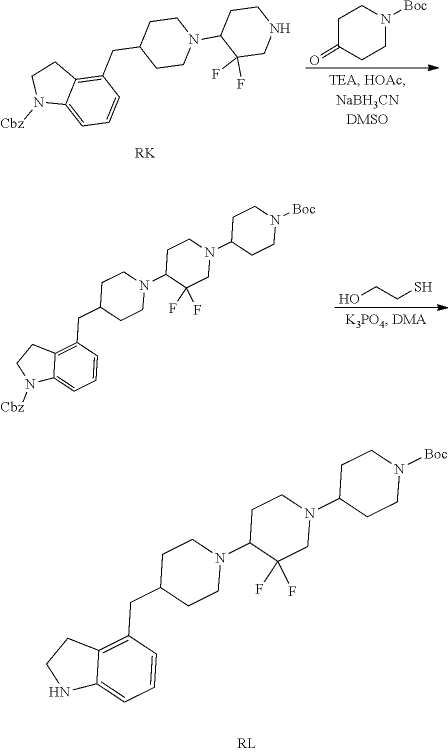

Step 1—Tert-butyl 4-[4-({1-[(benzyloxy)carbonyl]-2,3-dihydroindol-4-yl}methyl)piperidin-1-yl]-3,3-difluoro-[1,4′-bipiperidine]-1′-carboxylate. To a stirred mixture of benzyl 4-({3′,3′-difluoro-[1,4′-bipiperidin]-4-yl}methyl)-2,3-dihydroindole-1-carboxylate (500 mg, 1.07 mmol, Intermediate RK) and TEA (0.44 mL, 3.20 mmol) in DMSO (5 mL) were added tert-butyl 4-oxopiperidine-1-carboxylate (318.24 mg, 1.597 mmol) and HOAc (0.31 mL, 5.33 mmol) at rt. The resulting mixture was stirred for 30 min at 50° C. The mixture was allowed to cool to rt. To the above mixture was added NaBH₃CN (133.82 mg, 2.130 mmol) at rt and the mixture was stirred for 2 h at 50° C. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 80%-100% B in 15 min; Flow rate: 80 mL/min; Detector: 220/254 nm; desired fractions were collected at 100% B) and concentrated under reduced pressure to afford the title compound (300 mg, 43% yield) as a yellow oil. LC/MS (ESI, m/z): [(M+H)]⁺=653.4.

Step 2—Tert-butyl 4-[4-(2,3-dihydro-1H-indol-4-ylmethyl)piperidin-1-yl]-3,3-difluoro-[1,4′-bipiperidine]-1′-carboxylate. To a stirred mixture of tert-butyl 4-[4-({1-[(benzyloxy)carbonyl]-2,3-dihydroindol-4-yl}methyl)piperidin-1-yl]-3,3-difluoro-[1,4′-bipiperidine]-1′-carboxylate (279 mg, 0.427 mmol) and (3-mercaptoethanol (66.78 mg, 0.854 mmol) in DMA (3 mL) was added K₃PO₄ (297.75 mg, 1.708 mmol) at rt. The resulting mixture was stirred for 16 h at 70° C. On completion, the mixture was cooled to rt and purified by reverse phase flash chromatography (Column: WelFlash™ C18-I, 20-40 μm, 120 g; Eluent A: Water (plus 10 mmol/L NH₄HCO₃); Eluent B: ACN; Gradient: 80%-95% B in 25 min; Flow rate: 60 mL/min; Detector: 220/254 nm; desired fractions were collected at 85% B) and concentrated under reduced pressure to the title compound (200 mg, 90% yield) as a yellow oil. LC/MS (ESI, m/z): [(M+H)]⁺=519.3.

### 6-{4-[(1-{3,3-Difluoro-[1,4′-bipiperidin]-4-yl}piperidin-4-yl)methyl]-2,3-dihydroindol-1-yl}-N-[(1R,2S)-2-fluorocyclopropyl]-8-(methylamino)imidazo[1,2-b]pyridazine-3-carboxamide trifluoroacetate (Intermediate RM)

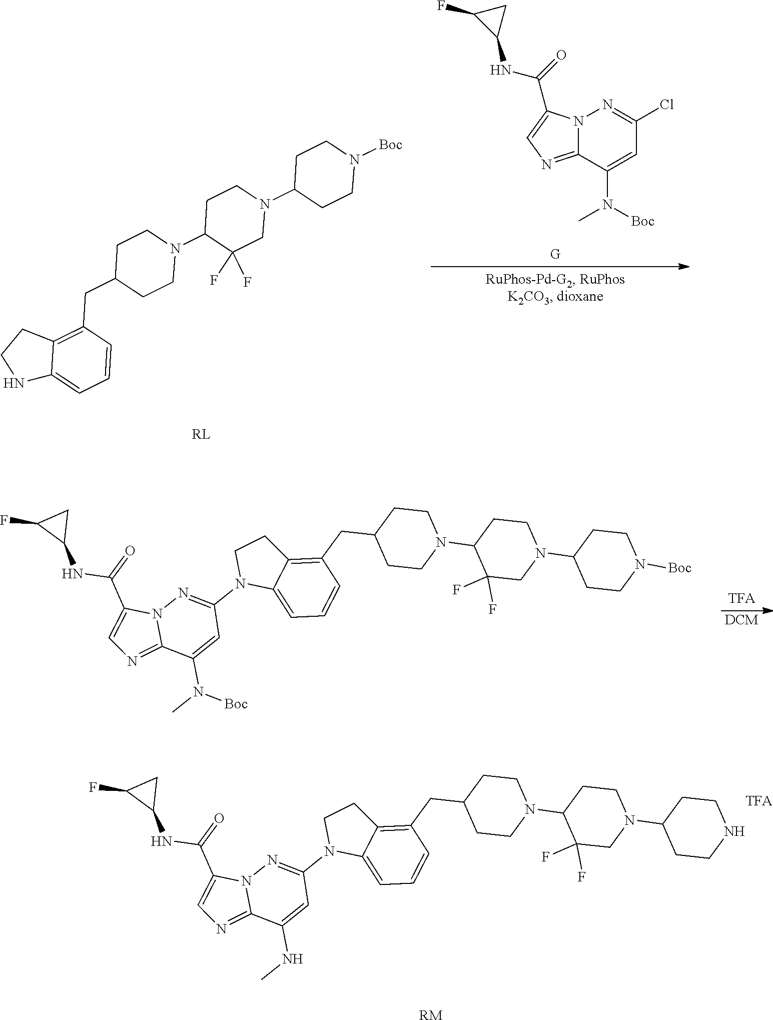

Step 1—Tert-butyl 4-{4-[(1-{8-[(tert-butoxycarbonyl)(methyl)amino]-3-{[(1R,2S)-2-fluorocyclopropyl]carbamoyl}imidazo[1,2-b]pyridazin-6-yl}-2,3-dihydroindol-4-yl)methyl]piperidin-1-yl}-3,3-difluoro-[1,4′-bipiperidine]-1′-carboxylate. To a stirred mixture of tert-butyl 4-[4-(2,3-dihydro-1H-indol-4-ylmethyl)piperidin-1-yl]-3,3-difluoro-[1,4′-bipiperidine]-1′-carboxylate (200 mg, 0.386 mmol, Intermediate RL) and tert-butyl N-(6-chloro-3-{[(1R,2S)-2-fluorocyclopropyl]carbamoyl}imidazo[1,2-b]pyridazin-8-yl)-N-methylcarbamate (177.59 mg, 0.463 mmol, Intermediate G) in dioxane (3 mL) were added K₂CO₃ (159.87 mg, 1.158 mmol), RuPhos (35.99 mg, 0.077 mmol) and RuPhos-PdCl-2nd G (59.98 mg, 0.077 mmol) at rt under nitrogen atmosphere. The resulting mixture was stirred for 2 h at 100° C. under nitrogen atmosphere. On completion, the mixture was concentrated under reduced pressure. The residue was purified by silica gel column chromatography, eluted with CH₂Cl₂/EA(10:1˜1:2), to afford the title compound (200 mg, 60% yield) as a white solid. LC/MS (ESI, m/z): [(M+H)]⁺=866.4.

Step 2—6-{4-[(1-{3,3-Difluoro-[1,4′-bipiperidin]-4-yl}piperidin-4-yl)methyl]-2,3-dihydroindol-1-yl}-N-[(1R,2S)-2-fluorocyclopropyl]-8-(methylamino)imidazo[1,2-b]pyridazine-3-carboxamide trifluoroacetate. To a stirred solution of tert-butyl 4-{4-[(1-{8-[(tert-butoxycarbonyl)(methyl)amino]-3-{[(1R,2S)-2-fluorocyclopropyl]carbamoyl}imidazo[1,2-b]pyridazin-6-yl}-2,3-dihydroindol-4-yl)methyl]piperidin-1-yl}-3,3-difluoro-[1,4′-bipiperidine]-1′-carboxylate (300 mg, 0.346 mmol) in DCM (3 mL) was added TFA (3 mL) at rt and the mixture was stirred for 1 h at rt. On completion, the mixture was concentrated under reduced pressure. The residue was purified by trituration with Et₂O (10 mL) to afford the title compound (230 mg) as a yellow solid. LC/MS (ESI, m/z): [(M+H)]⁺=666.3.

### N-(3-{6-[(3R)-3-methoxyoxolan-3-yl]-4-methylpyridin-2-yl}-1-(piperidin-4-yl)pyrrolo[2,3-c]pyridin-5-yl)acetamide trifluoroacetate (Intermediate RN)

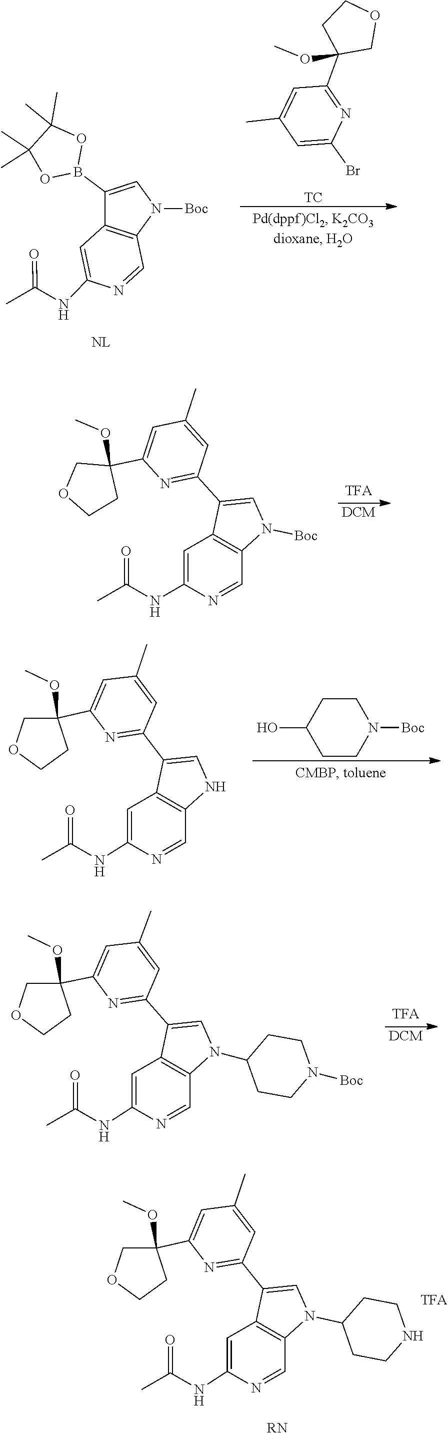

Step 1—Tert-butyl 5-acetamido-3-{6-[(3R)-3-methoxyoxolan-3-yl]-4-methylpyridin-2-yl}pyrrolo[2,3-c]pyridine-1-carboxylate. To a stirred mixture of tert-butyl 5-acetamido-3-(4,4,5,5-tetramethyl-1,3,2-dioxaborolan-2-yl)pyrrolo[2,3-c]pyridine-1-carboxylate (1.00 g, 2.49 mmol, Intermediate NL) and K₂CO₃ (1033 mg, 7.474 mmol) in dioxane (10 mL) and H₂O (1 mL) were added 2-bromo-6-[(3R)-3-methoxyoxolan-3-yl]-4-methylpyridine (678 mg, 2.49 mmol, Intermediate TC) and Pd(dppf)Cl₂CH₂Cl₂ (203 mg, 0.249 mmol) at rt under nitrogen atmosphere. The resulting mixture was stirred for 1 h at 90° C. under nitrogen atmosphere. On completion, the mixture was cooled to rt and concentrated under reduced pressure. The resulting mixture was diluted with water (50 mL) and extracted with EtOAc (3×50 mL). The combined organic layers were washed with brine (1×100 mL), and dried over anhydrous Na₂SO₄. After filtration, the filtrate was concentrated under reduced pressure. The residue was purified by reverse phase flash chromatography (Column: Spherical C18, 20-40 μm, 330 g; Mobile Phase A: Water (plus 10 mM NH₄HCO₃); Mobile Phase B: ACN; Flow rate: 80 mL/min; Gradient: 50% B-70% B in 20 min; Detector: 254 nm; the fractions containing desired product were collected at 62% B and concentrated under reduced pressure) to afford the title compound (800 mg, 69%) as a yellow solid. LC/MS (ESI, m/z): [(M+H)]⁺=467.2.

Step 2—N-(3-{6-[(3R)-3-methoxyoxolan-3-yl]-4-methylpyridin-2-yl}-1H-pyrrolo[2,3-c]pyridin-5-yl)acetamide. A solution of tert-butyl 5-acetamido-3-{6-[(3R)-3-methoxyoxolan-3-yl]-4-methylpyridin-2-yl}pyrrolo[2,3-c]pyridine-1-carboxylate (800 mg, 1.72 mmol) in TFA (4 mL) and DCM (4 mL) was stirred for 1 h at rt under nitrogen atmosphere. The resulting mixture was concentrated under reduced pressure. The residue was dissolved in acetonitrile (20 mL). The mixture was basified to pH 8 with TEA. The mixture was purified by reverse phase flash chromatography (Column: Spherical C18, 20-40 μm, 330 g; Mobile Phase A: Water (plus 10 mM NH₄HCO₃); Mobile Phase B: ACN; Flow rate: 80 mL/min; Gradient: 25% B-45% B in 20 min; Detector: 254 nm; the fractions containing desired product were collected at 39% B and concentrated under reduced pressure) to afford the title compound (570 mg, 91% yield) as a white solid. LC/MS (ESI, m/z): [(M+H)]⁺=367.2.

Step 3—Tert-butyl 4-(5-acetamido-3-{6-[(3R)-3-methoxyoxolan-3-yl]-4-methylpyridin-2-yl}pyrrolo[2,3-c]pyridin-1-yl)piperidine-1-carboxylate. To a stirred mixture of N-(3-{6-[(3R)-3-methoxyoxolan-3-yl]-4-methylpyridin-2-yl}-1H-pyrrolo[2,3-c]pyridin-5-yl)acetamide (500 mg, 1.37 mmol) and tert-butyl 4-hydroxypiperidine-1-carboxylate (549 mg, 2.73 mmol) in toluene (5 mL) was added 2-(tributyl-l-{5}-phosphanylidene)acetonitrile (494 mg, 2.05 mmol) at rt under nitrogen atmosphere. The resulting mixture was stirred for 1 h at 120° C. under nitrogen atmosphere. On completion, the mixture was cooled to rt and concentrated under reduced pressure. The reside was purified by reverse phase flash chromatography (Column: Spherical C18, 20-40 μm, 330 g; Mobile Phase A: Water (plus 10 mM NH₄HCO₃); Mobile Phase B: ACN; Flow rate: 80 mL/min; Gradient: 30% B-50% B in 20 min; Detector: 254 nm; the fractions containing desired product were collected at 43% B and concentrated under reduced pressure) to afford the title compound (620 mg, 83% yield) as a white solid. LC/MS (ESI, m/z): [(M+H)]+=550.3.

Step 4—N-(3—{6-[(3R)-3-methoxyoxolan-3-yl]-4-methylpyridin-2-yl}-1-(piperidin-4-yl)pyrrolo[2,3-c]pyridin-5-yl)acetamide trifluoroacetate. A solution of tert-butyl 4-(5-acetamido-3-{6-[(3R)-3-methoxyoxolan-3-yl]-4-methylpyridin-2-yl}pyrrolo[2,3-c]pyridin-1-yl)piperidine-1-carboxylate (620 mg, 1.13 mmol) in TFA (3 mL) and DCM (6 mL) was stirred for 1 h at rt under nitrogen atmosphere. On completion, the mixture was concentrated under reduced pressure. The residue was purified by trituration with diethyl ether (20 mL) to give the title compound (500 mg, 81% yield) as alight green solid. LC/MS (ESI, m/z): [(M+H)]⁺=450.2.

### 3-[3-Methyl-2-oxo-5-(4-oxopiperidin-1-yl)-1,3-benzodiazol-1-yl]piperidine-2,6-dione (Intermediate RO)

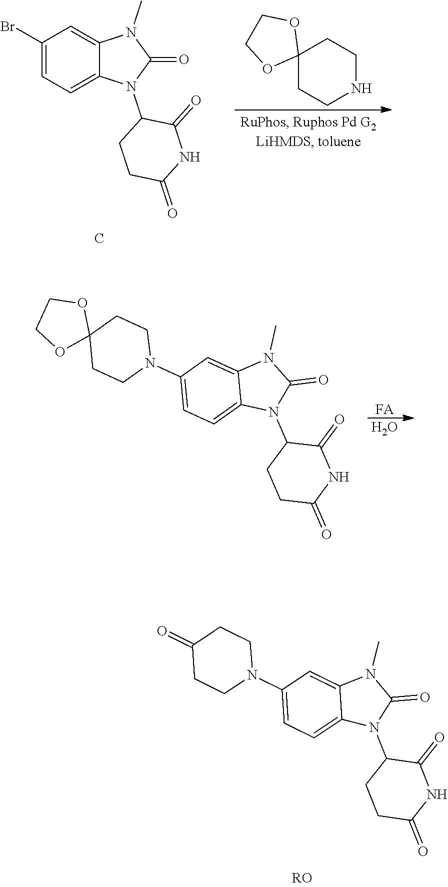

Step 1—3-(5-{1,4-Dioxa-8-azaspiro[4.5]decan-8-yl}-3-methyl-2-oxo-1,3-benzodiazol-1-yl)piperidine-2,6-dione. To a stirred mixture of 3-(5-bromo-3-methyl-2-oxo-1,3-benzodiazol-1-yl)piperidine-2,6-dione (1 g, 3 mmol, Intermediate C) and 1,4-dioxa-8-azaspiro[4.5]decane (0.42 g, 3.0 mmol) in toluene (30 mL) were added RuPhos (0.14 g, 0.30 mmol), RuPhos-PdCl-2nd G (0.23 g, 0.30 mmol) and LiHMDS (24 mL, 36 mmol) at rt under nitrogen atmosphere. The resulting mixture was stirred for 1 h at 80° C. On completion, the mixture was cooled to rt and acidified to pH 5 with FA. The resulting mixture was concentrated under reduced pressure. The residue was purified by silica gel column chromatography, eluted with EA, to afford the title compound (1 g, 85% yield) as a yellow solid. LC/MS (ESI, m/z): [(M+H)]⁺=401.1.

Step 2—3-[3-Methyl-2-oxo-5-(4-oxopiperidin-1-yl)-1,3-benzodiazol-1-yl]piperidine-2,6-dione. To a stirred mixture of 3-(5-{1,4-dioxa-8-azaspiro[4.5]decan-8-yl}-3-methyl-2-oxo-1,3-benzodiazol-1-yl)piperidine-2,6-dione (1 g, 3 mmol) in FA (10 mL) was added H₂O (10 mL) at rt under nitrogen atmosphere. The resulting mixture was stirred for 1 h at 100° C. The On completion, the mixture was cooled to rt and basified to pH 8 with saturated Na₂CO₃ (aq.). The resulting mixture was extracted with EtOAc (3×50 mL). The combined organic layers were dried over anhydrous Na₂SO₄, filtered, and the filtrate was concentrated under reduced pressure to give the title compound (700 mg, 79% yield) as a yellow solid. LC/MS (ESI, m/z): [(M+H)]⁺=357.1.

### (S)-1-(2-chloro-5-(4-(3,3-difluoropiperidin-4-yl)piperazine-1-carbonyl)phenyl)dihydropyrimidine-2,4(1H,3H)-dione 2,2,2-trifluoroacetate (Intermediate RP)

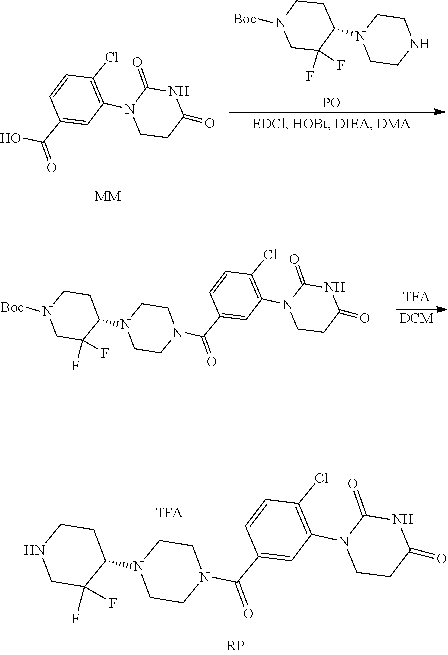

Step 1—Tert-butyl (S)-4-(4-(4-chloro-3-(2,4-dioxotetrahydropyrimidin-1(2H)-yl)benzoyl)piperazin-1-yl)-3,3-difluoropiperidine-1-carboxylate. To a stirred solution of 4-chloro-3-(2,4-dioxotetrahydropyrimidin-1(2H)-yl)benzoic acid (600 mg, 2.23 mmol, Intermediate MM) and DIEA (865 mg, 6.70 mmol) in DMA (10 mL) were added EDCI (642 mg, 3.35 mmol), HOBT (452 mg, 3.35 mmol) and tert-butyl (4S)-3,3-difluoro-4-(piperazin-1-yl)piperidine-1-carboxylate (682 mg, 2.23 mmol, Intermediate PO) at rt and the mixture was stirred for 1 h at rt. On completion, the mixture was purified directly by reversed-phase flash chromatography (Column: Spherical C18, 20-40 μm, 330 g; Mobile Phase A: water (plus 10 mM NH₄HCO₃), Mobile Phase B: ACN; Flow rate: 80 mL/min; Gradient (B %): 5%-30%, 4 min; 30%-70%, 20 min; 70%-95%; 2 min; 95%, 5 min; Detector: 254 nm; the fractions containing desired product were collected at 60% B and concentrated under reduced pressure) to afford the title compound (882 mg, 71% yield) as a yellow solid. LC/MS (ESI, m/z): [(M+1)]⁺=556.2.

Step 2—(S)-1-(2-chloro-5-(4-(3,3-difluoropiperidin-4-yl)piperazine-1-carbonyl)phenyl)dihydropyrimidine-2,4(1H,3H)-dione 2,2,2-trifluoroacetate. A solution of tert-butyl (S)-4-(4-(4-chloro-3-(2,4-dioxotetrahydropyrimidin-1(2H)-yl)benzoyl)piperazin-1-yl)-3,3-difluoropiperidine-1-carboxylate (880 mg, 1.58 mmol) in DCM (20 mL) and TFA (5 mL) was stirred for 1 h at rt. On completion, the mixture was concentrated under reduced pressure. The resulting mixture was diluted with diethyl ether (10 mL). The precipitated solids were collected by filtration and washed with diethyl ether (2×10 mL) to give the title compound (1.2 g, TFA salt) as a white solid. LC/MS (ESI, m/z): [(M+1)]+=456.4.

### 2-(Indolin-4-yl)pyrimidine-5-carbaldehyde (Intermediate RO)

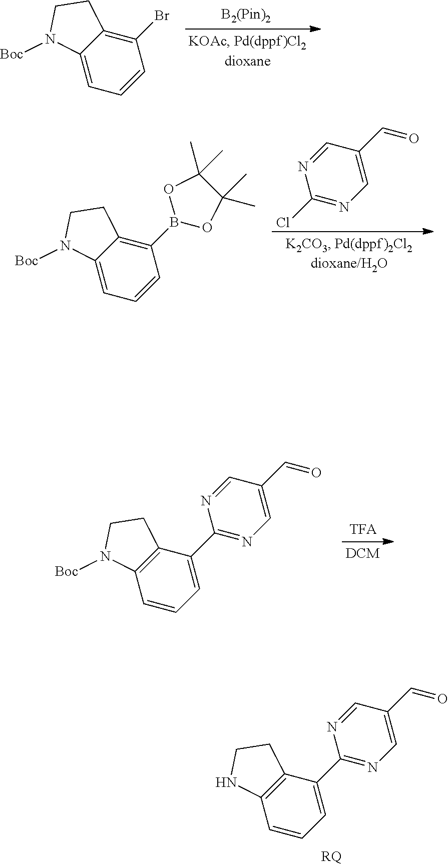

Step 1—Tert-butyl 4-(4,4,5,5-tetramethyl-1,3,2-dioxaborolan-2-yl)-2,3-dihydroindole-1-carboxylate. To a stirred solution of tert-butyl 4-bromo-2,3-dihydroindole-1-carboxylate (25.00 g, 83.84 mmol) and bis(pinacolato)diboron (31.94 g, 125.8 mmol) in dioxane (250 mL) were added KOAc (24.69 g, 251.5 mmol) and Pd(dppf)Cl₂·CH₂Cl₂ (6.83 g, 8.38 mmol) at rt under nitrogen atmosphere. The resulting mixture was stirred for 2 h at 90° C. under nitrogen atmosphere. On completion, the mixture was cooled to rt and concentrated under reduced pressure. The residue was purified by silica gel column chromatography, eluted with PE/EA (10:1) to afford the title compound (27.92 g, 97% yield) as a light yellow solid. LC/MS (ESI, m/z): [(M+H)]⁺=346.2.

Step 2—Tert-butyl 4-(5-formylpyrimidin-2-yl)indoline-1-carboxylate. To a stirred mixture of tert-butyl 4-(4,4,5,5-tetramethyl-1,3,2-dioxaborolan-2-yl)-2,3-dihydroindole-1-carboxylate (3 g, 9 mmol) and 2-chloropyrimidine-5-carbaldehyde (1.24 g, 8.69 mmol) in dioxane (45 mL) and H₂O (9 mL) were added K₂CO₃ (3.60 g, 26.1 mmol) and Pd(dppf)Cl₂CH₂Cl₂ (1.42 g, 1.74 mmol) at rt under nitrogen atmosphere. The resulting mixture was stirred for 16 h at 90° C. under nitrogen atmosphere. On completion, the mixture was cooled to rt and concentrated under vacuum. The residue was purified by reverse phase flash chromatography (Column: Spherical C18, 20-40 μm, 330 g; Mobile Phase A: Water (plus 10 mM NH₄HCO₃); Mobile Phase B: ACN; Flow rate: 80 mL/min; Gradient: 60% B-80% B in 20 min; Detector: 254 nm; the fractions containing desired product were collected at 70% B and concentrated under reduced pressure) to give the title compound (290 mg, 10% yield) as a yellow solid. LC/MS (ESI, m/z): [(M+H)]+=326.1.

Step 3—2-(Indolin-4-yl)pyrimidine-5-carbaldehyde. To a stirred solution of tert-butyl 4-(5-formylpyrimidin-2-yl)indoline-1-carboxylate (200 mg, 0.615 mmol) in DCM (5 mL) was added TFA (1 mL) at rt and the mixture was stirred for 1 h at it. On completion, the mixture was basified to pH 8 with saturated Na₂CO₃ (aq.). The resulting mixture was extracted with EtOAc (3×25 mL). The combined organic layers were washed with brine (1×50 mL), dried over anhydrous Na₂SO₄. After filtration, the filtrate was concentrated under reduced pressure to give the title compound (70 mg, 510% yield) as a yellow solid. LC/MS (ESI, m/z): [(M+H)]f=226.0.

### 6-(4-(5-formylpyrimidin-2-yl)indolin-1-yl)-N-((1R,2R)-2-methoxycyclobutyl)-8-(methylamino)imidazo[1,2-b]pyridazine-3-carboxamide trifluoroacetate (Intermediate RR)

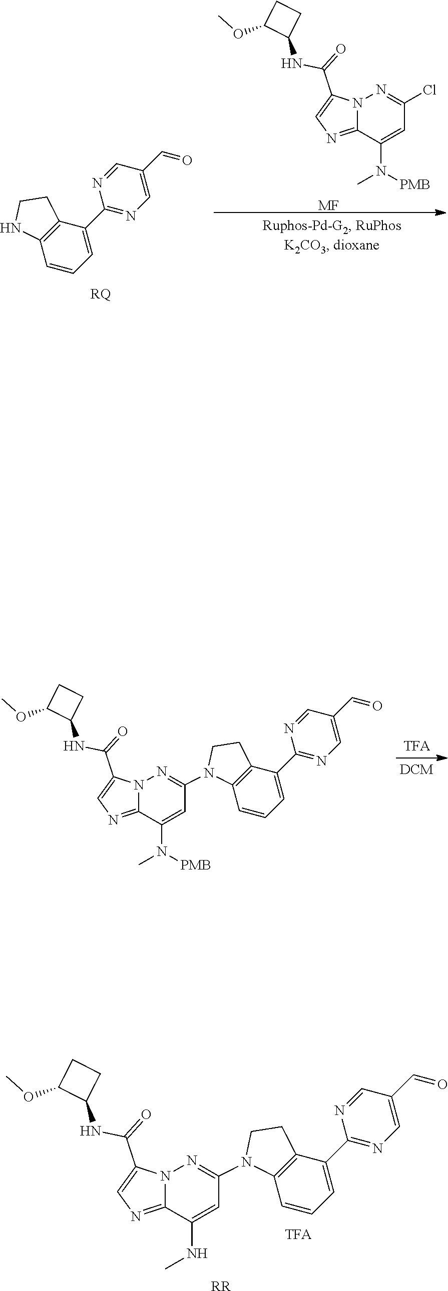

Step 1—6-(4-(5-Formylpyrimidin-2-yl)indolin-1-yl)-8-((4-methoxybenzyl)(methyl)amino)-N-((1R,2R)-2-methoxycyclobutyl)imidazo[1,2-b]pyridazine-3-carboxamide. To a stirred mixture of 2-(indolin-4-yl)pyrimidine-5-carbaldehyde (70 mg, 0.31 mmol, Intermediate RQ) and 6-chloro-N-[(1R,2R)-2-methoxycyclobutyl]-8-{[(4-methoxyphenyl)methyl](methyl)amino}imidazo[1,2-b]pyridazine-3-carboxamide (133.60 mg, 0.311 mmol, Intermediate MF) in dioxane (3 mL) were added K₂CO₃ (128.85 mg, 0.933 mmol), Ruphos (28.89 mg, 0.062 mmol) and RuPhos-PdCl-2nd G (48.34 mg, 0.062 mmol) at rt under nitrogen atmosphere. The resulting mixture was stirred for 2 h at 100° C. under nitrogen atmosphere. On completion, the mixture was concentrated under vacuum. The residue was purified by reverse phase flash chromatography (Column: Spherical C18, 20-40 μm, 120 g; Mobile Phase A: Water (plus 10 mM NH₄HCO₃); Mobile Phase B: ACN; Flow rate: 60 mL/min; Gradient: 60% B-80% B in 20 min; Detector: 254 nm; the fractions containing desired product were collected at 65% B and concentrated under reduced pressure) to give the title compound (60 mg, 31% yield) as a yellow solid. LC/MS (ESI, m/z): [(M+H)]⁺=619.2.

Step 2—6-(4-(5-Formylpyrimidin-2-yl)indolin-1-yl)-N-((1R,2R)-2-methoxycyclobutyl)-8-(methylamino)imidazo[1,2-b]pyridazine-3-carboxamide trifluoroacetate. To a stirred solution of 6-(4-(5-formylpyrimidin-2-yl)indolin-1-yl)-8-((4-methoxybenzyl)(methyl)amino)-N-((1R,2R)-2-methoxycyclobutyl)imidazo[1,2-b]pyridazine-3-carboxamide (60 mg, 0.097 mmol) in DCM (1 mL) were added TFA (0.5 mL) at rt and the mixture was stirred for 1 h at rt. On completion, the mixture was concentrated under vacuum. The residue was purified by trituration with diethyl ether (10 mL) to afford the title compound (75 mg) as a yellow solid. LC/MS (ESI, m/z): [(M+H)]⁺=499.2.

### 6-(4-(5-((1,1-difluoro-3,9-diazaspiro[5.5]undecan-3-yl)methyl)pyrimidin-2-yl)indolin-1-yl)-N-((1R,2R)-2-methoxycyclobutyl)-8-(methylamino)imidazo[1,2-b]pyridazine-3-carboxamide trifluoroacetate (Intermediate RS)

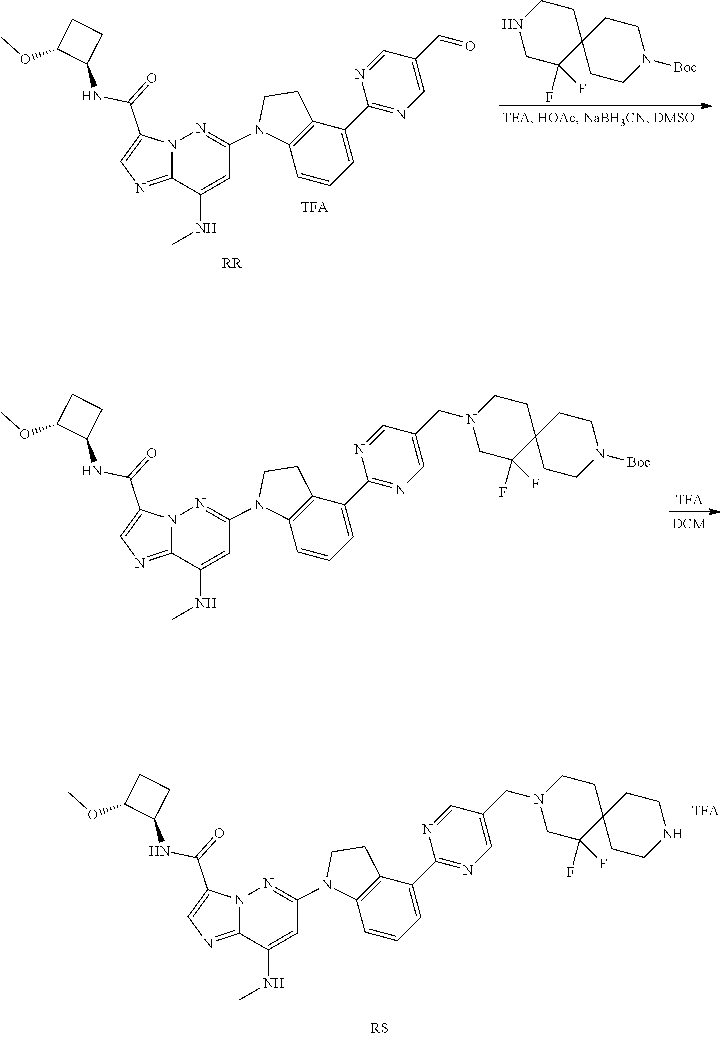

Step 1—Tert-butyl 7,7-difluoro-9-((2-(1-(3-(((1R,2R)-2-methoxycyclobutyl)carbamoyl)-8-(methylamino)imidazo[1,2-b]pyridazin-6-yl)indolin-4-yl)pyrimidin-5-yl)methyl)-3,9-diazaspiro[5.5]undecane-3-carboxylate. To a stirred mixture of tert-butyl 7,7-difluoro-3,9-diazaspiro[5.5]undecane-3-carboxylate (34.07 mg, 0.117 mmol) and Et₃N (0.01 mL) in DMSO (2 mL) were added 6-(4-(5-formylpyrimidin-2-yl)indolin-1-yl)-N-((1R,2R)-2-methoxycyclobutyl)-8-(methylamino)imidazo[1,2-b]pyridazine-3-carboxamide trifluoroacetate (70 mg, 0.12 mmol. Intermediate RR) and HOAc (0.02 mL) at rt. The resulting mixture was stirred for 30 min at 50° C. The mixture was allowed to cool down to rt. To the above mixture was added NaBH₃CN (36.87 mg, 0.587 mmol) at it then the mixture was stirred for an additional 2 h at 50° C. On completion, the mixture was cooled to it and concentrated under vacuum. The residue was purified by reverse phase flash chromatography (Column: Spherical C18, 20-40 μm, 120 g; Mobile Phase A: Water (plus 10 mM NH₄HCO₃); Mobile Phase B: ACN; Flow rate: 60 mL/min; Gradient: 60% B-80% B in 20 min; Detector: 254 nm; the fractions containing desired product were collected at 70% B and concentrated under reduced pressure) to give the title compound (20 mg, 22% yield) as a white solid. LC/MS (ESI, m/z): [(M+H)]⁺=773.4.

Step 2—6-(4-(5-((1,1-Difluoro-3,9-diazaspiro[5.5]undecan-3-yl)methyl)pyrimidin-2-yl)indolin-1-yl)-N-((1R,2R)-2-methoxycyclobutyl)-8-(methylamino)imidazo[1,2-b]pyridazine-3-carboxamide trifluoroacetate. To a stirred solution of tert-butyl 7,7-difluoro-9-((2-(1-(3-(((1R,2R)-2-methoxycyclobutyl)carbamoyl)-8-(methylamino)imidazo[1,2-b]pyridazin-6-yl)indolin-4-yl)pyrimidin-5-yl)methyl)-3,9-diazaspiro[5.5]undecane-3-carboxylate (20 mg, 0.026 mmol) in DCM (1 mL) was added TFA (0.5 mL) at it and the mixture was stirred for 1 h at rt. On completion, the mixture was concentrated under vacuum. The residue was purified by trituration with diethyl ether (10 mL) to afford the title compound (20 mg) as a yellow solid. LC/MS (ESI, m/z): [(M+H)]⁺=673.3.

### N-((1R,2S)-2-fluorocyclopropyl)-6-(4-(5-formylpyrimidin-2-yl)indolin-1-yl)-8-(methylamino)imidazo[1,2-b]pyridazine-3-carboxamide (Intermediate RT)

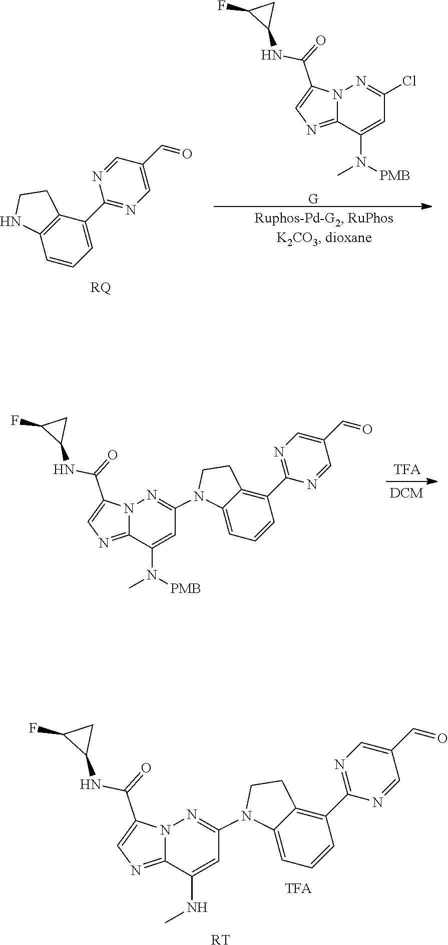

Step 1—N-((1R,2S)-2-fluorocyclopropyl)-6-(4-(5-formylpyrimidin-2-yl)indolin-1-yl)-8-((4-methoxybenzyl)(methyl)amino)imidazo[1,2-b]pyridazine-3-carboxamide. To a stirred mixture of 2-(2,3-dihydro-1H-indol-4-yl)pyrimidine-5-carbaldehyde (120 mg, 0.533 mmol, Intermediate RQ) and 6-chloro-N-[(1R,2S)-2-fluorocyclopropyl]-8-{[(4-methoxyphenyl)methyl](methyl)amino}imidazo[1,2-b]pyridazine-3-carboxamide (215.14 mg, 0.533 mmol, Intermediate G) in dioxane (5 mL) were added K₂CO₃ (220.88 mg, 1.599 mmol) at rt. To the above mixture was added RuPhos (49.72 mg, 0.107 mmol) and RuPhos-PdCl-2nd G (82.87 mg, 0.107 mmol) at rt under nitrogen atmosphere. The resulting mixture was stirred for 2 h at 110° C. On completion, the mixture was cooled to rt and concentrated under vacuum. The residue was purified by reverse phase flash chromatography (Column: Spherical C18, 20-40 μm, 330 g; Mobile Phase A: Water (plus 10 mM NH₄HCO₃); Mobile Phase B: ACN; Flow rate: 80 mL/min; Gradient: 55% B-75% B in 20 min; Detector: 254 nm; the fractions containing desired product were collected at 65% B and concentrated under reduced pressure) to give the title compound (80 mg, 25 yield %) as a yellow solid. LC/MS (ESI, m/z): [(M+H)]⁺=593.2.

Step 2—N-((1R,2S)-2-fluorocyclopropyl)-6-(4-(5-formylpyrimidin-2-yl)indolin-1-yl)-8-(methylamino)imidazo[1,2-b]pyridazine-3-carboxamide trifluoroacetate. To a stirred solution of N-((1R,2S)-2-fluorocyclopropyl)-6-(4-(5-formylpyrimidin-2-yl)indolin-1-yl)-8-((4-methoxybenzyl)(methyl)amino)imidazo[1,2-b]pyridazine-3-carboxamide (80 mg, 0.135 mmol) in DCM (2 mL) was added TFA (1 mL) at rt and the mixture was stirred for 1 h at rt. On completion, the mixture was concentrated under vacuum. The residue was purified by trituration with diethyl ether (25 mL) to afford the title compound (110 mg) as a yellow solid. LC/MS (ESI, m/z): [(M+H)]⁺=473.0.

### 6-(4-(5-((1,1-Difluoro-3,9-diazaspiro[5.5]undecan-3-yl)methyl)pyrimidin-2-yl)indolin-1-yl)-N-((1R,2S)-2-fluorocyclopropyl)-8-(methylamino)imidazo[1,2-b]pyridazine-3-carboxamide trifluoroacetate (Intermediate RU)

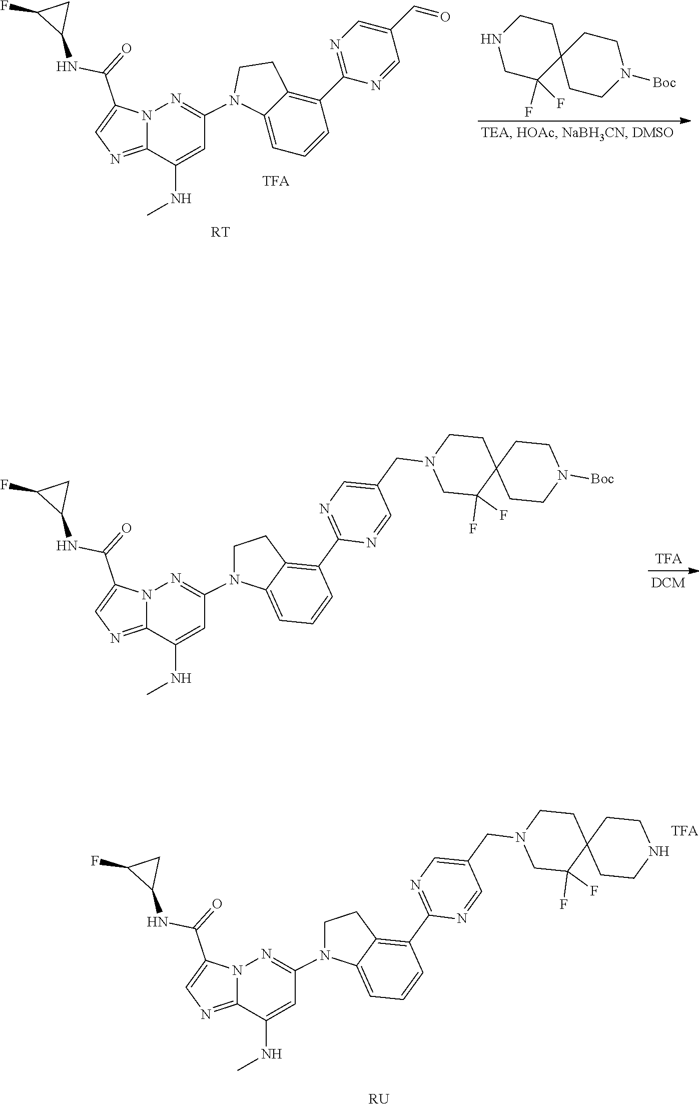

Step 1—Tert-butyl 7,7-difluoro-9-((2-(1-(3-(((1R,2S)-2-fluorocyclopropyl)carbamoyl)-8-(methylamino)imidazo[1,2-b]pyridazin-6-yl)indolin-4-yl)pyrimidin-5-yl)methyl)-3,9-diazaspiro[5.5]undecane-3-carboxylate. To a stirred mixture of tert-butyl 7,7-difluoro-3,9-diazaspiro[5.5]undecane-3-carboxylate (55.98 mg, 0.193 mmol, CAS #1784848-04-9) and Et₃N (0.02 mL, 0,144 mmol) in DMSO (2 mL) were added N-((1R,2S)-2-fluorocyclopropyl)-6-(4-(5-formylpyrimidin-2-yl)indolin-1-yl)-8-(methylamino)imidazo[1,2-b]pyridazine-3-carboxamide trifluoroacetate (110 mg, 0.193 mmol, Intermediate RT) and HOAc (0.03 mL, 0.579 mmol) at rt. The resulting mixture was stirred for 30 min at 50° C. The mixture was allowed to cool down to rt. To the above mixture was added NaBH₃CN (60.58 mg, 0.965 mmol) at rt then the mixture was stirred for an additional 2 h at 50° C. On completion, the mixture was concentrated under vacuum. The residue was purified by reverse phase flash chromatography (Column: Spherical C18, 20-40 μm, 120 g; Mobile Phase A: Water (plus 10 mM NH₄HCO₃); Mobile Phase B: ACN; Flow rate: 60 mL/min; Gradient: 60% B-80% B in 20 min; Detector: 254 nm; the fractions containing desired product were collected at 70% B and concentrated under reduced pressure) to give the title compound (40 mg, 28% yield) as a white solid. LC/MS (ESI, m/z): [(M+H)]⁺=747.2.

Step 2—6-(4-(5-((1,1-Difluoro-3,9-diazaspiro[5.5]undecan-3-yl)methyl)pyrimidin-2-yl)indolin-1-yl)-N-((1R,2S)-2-fluorocyclopropyl)-8-(methylamino)imidazo[1,2-b]pyridazine-3-carboxamide trifluoroacetate. To a stirred solution of tert-butyl 7,7-difluoro-9-((2-(1-(3-(((1R,2S)-2-fluorocyclopropyl)carbamoyl)-8-(methylamino)imidazo[1,2-b]pyridazin-6-yl)indolin-4-yl)pyrimidin-5-yl)methyl)-3,9-diazaspiro[5.5]undecane-3-carboxylate (40 mg, 0.054 mmol) in DCM (1 mL) was added TFA (0.3 mL) at rt and the mixture was stirred for 1 h at rt. On completion, the mixture was concentrated under vacuum. The residue was purified by trituration with diethyl ether (20 mL) to afford the title compound (30 mg, 75% yield) as a light red solid. LC/MS (ESI, m/z): [(M+H)]⁺=647.2.

### Tert-butyl N-(6-chloro-3-{[(1R,2S)-2-fluorocyclopropyl]carbamoyl}imidazo[1,2-b]pyridazin-8-yl)-N-methylcarbamate (Intermediate RV)

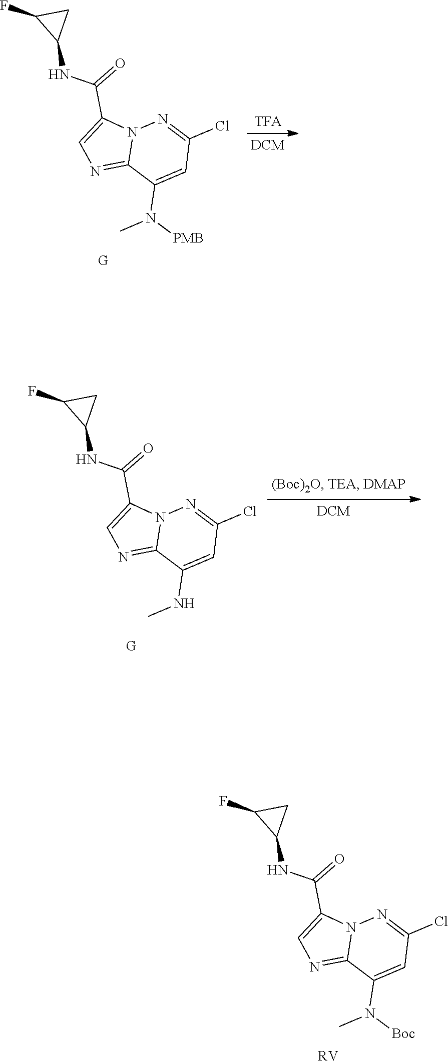

Step 1—6-Chloro-N-[(R,2S)-2-fluorocyclopropyl]-8-(methylamino)imidazo[1,2-b]pyridazine-3-carboxamide. To a solution of 6-chloro-N-[(1R,2S)-2-fluorocyclopropyl]-8-{[(4-methoxyphenyl)methyl](methyl)amino}imidazo[1,2-b]pyridazine-3-carboxamide (3 g, 7.429 mmol, Intermediate G) in DCM (60 mL) was added TFA (20 mL) dropwise at rt. The resulting solution was stirred for 1 h at rt. On completion, the resulting mixture was concentrated under reduced pressure. The resulting mixture was diluted with diethyl ether (20 mL). The precipitated solids were collected by filtration and washed with diethyl ether (2×10 mL) to afford the title compound (1.85 g, 88% yield) as a grey solid. LC/MS (ESI, m/z): [(M+1)]⁺=284.1.

Step 2—Tert-butyl N-(6-chloro-3-{[(1R,2S)-2-fluorocyclopropyl]carbamoyl}imidazo[1,2-b]pyridazin-8-yl)-N-methylcarbamate. To a stirred mixture of 6-chloro-N-[(1R,2S)-2-fluorocyclopropyl]-8-(methylamino)imidazo[1,2-b]pyridazine-3-carboxamide (1.85 g, 6.52 mmol) and TEA (1.98 g, 19.6 mmol) in DCM (50 mL) were added Boc₂O (1.71 g, 7.83 mmol) and DMAP (0.08 g, 0.65 mmol) in turns at rt. The resulting mixture was stirred for 30 min at rt. On completion, the resulting mixture was concentrated under reduced pressure. The resulting mixture was purified by silica gel column chromatography, eluted with PE/EA (1:1) to afford the title compound (2.5 g, quant, yield) as a yellow solid. LC/MS (ESI, m/z): [(M+1)]⁺=384.1

### N-cyclopropyl-6-[4-(5-formylpyridin-2-yl)-2,3-dihydroindol-1-yl]-8-(methylamino)imidazo[1,2-b]pyridazine-3-carboxamide (Intermediate RW)

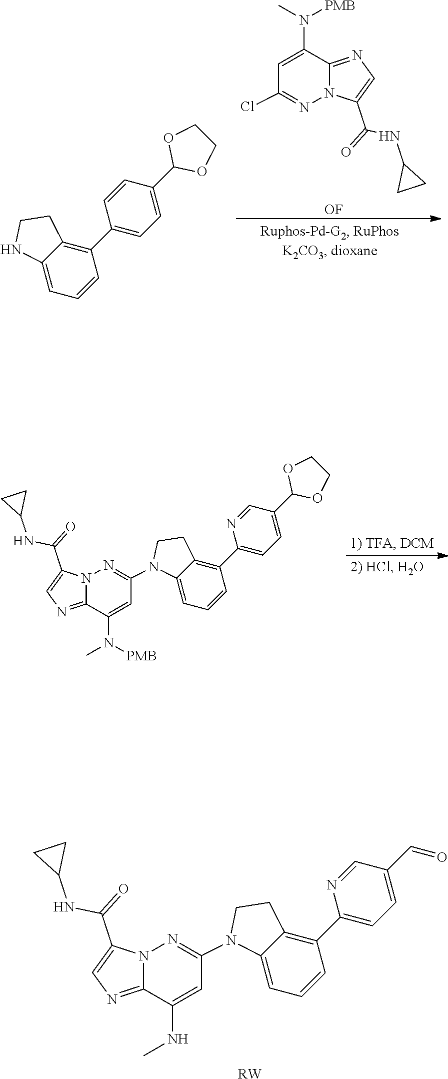

Step 1—N-cyclopropyl-6-{4-[5-(1,3-dioxolan-2-yl)pyridin-2-yl]-2,3-dihydroindol-1-yl}-8-{[(4-methoxyphenyl)methyl](methyl)amino}imidazo[1,2-b]pyridazine-3-carboxamide. To a stirred mixture of 4-[5-(1,3-dioxolan-2-yl)pyridin-2-yl]-2,3-dihydro-1H-indole (1.0 g, 3.7 mmol, synthesized via Steps 1-2 of Intermediate NP) and 6-chloro-N-cyclopropyl-8-{[(4-methoxyphenyl)methyl](methyl)amino}imidazo[1,2-b]pyridazine-3-carboxamide (1.44 g, 3.73 mmol, Intermediate OF) in 1,4-dioxane (20 mL) were added K₂CO₃ (1.55 g, 11.2 mmol), RuPhos (0.17 g, 0.37 mmol) and RuPhos-PdCl-2nd G (0.29 g, 0.37 mmol) at rt under nitrogen atmosphere. The resulting mixture was stirred for 1 h at 90° C. under nitrogen atmosphere. On completion, the mixture was cooled to rt and concentrated under reduced pressure. The residue was purified by silica gel column chromatography, eluted with CH₂Cl₂/EA (1:1) to afford the title compound (1.9 g, 83% yield) as a light yellow solid. LC/MS (ESI, m/z): [(M+H)]=618.2.

Step 2—N-cyclopropyl-6-[4-(5-formylpyridin-2-yl)-2,3-dihydroindol-1-yl]-8-(methylamino)imidazo[1,2-b]pyridazine-3-carboxamide. To a stirred solution of N-cyclopropyl-6-{4-[5-(1,3-dioxolan-2-yl)pyridin-2-yl]-2,3-dihydroindol-1-yl}-8-{[(4-methoxyphenyl)methyl](methyl)amino}imidazo[1,2-b]pyridazine-3-carboxamide (1.9 g, 3.1 mmol) in DCM (15 mL) was added TFA (5 mL) at rt under nitrogen atmosphere. The resulting mixture was stirred for 1 h at rt under nitrogen atmosphere. On completion, the mixture was concentrated under reduced pressure. To the above residue was added 3 M HCl (30 mL) at rt. The resulting mixture was stirred for additional 3 h at 60° C. On completion, the mixture was basified to pH 10 with NaOH (aq.). The precipitated solids were collected by filtration and washed with water (20 mL) to give the title compound (1.3 g, 93% yield) as a brown yellow solid. LC/MS (ESI, m/z): [(M+H)]⁺=454.1.

### N-cyclopropyl-6-(4-(5-((5,5-difluoro-2,7-diazaspiro[3.5]nonan-2-yl)methyl)pyridin-2-yl)indolin-1-yl)-8-(methylamino)imidazo[1,2-b]pyridazine-3-carboxamide trifluoroacetate (Intermediate RX)

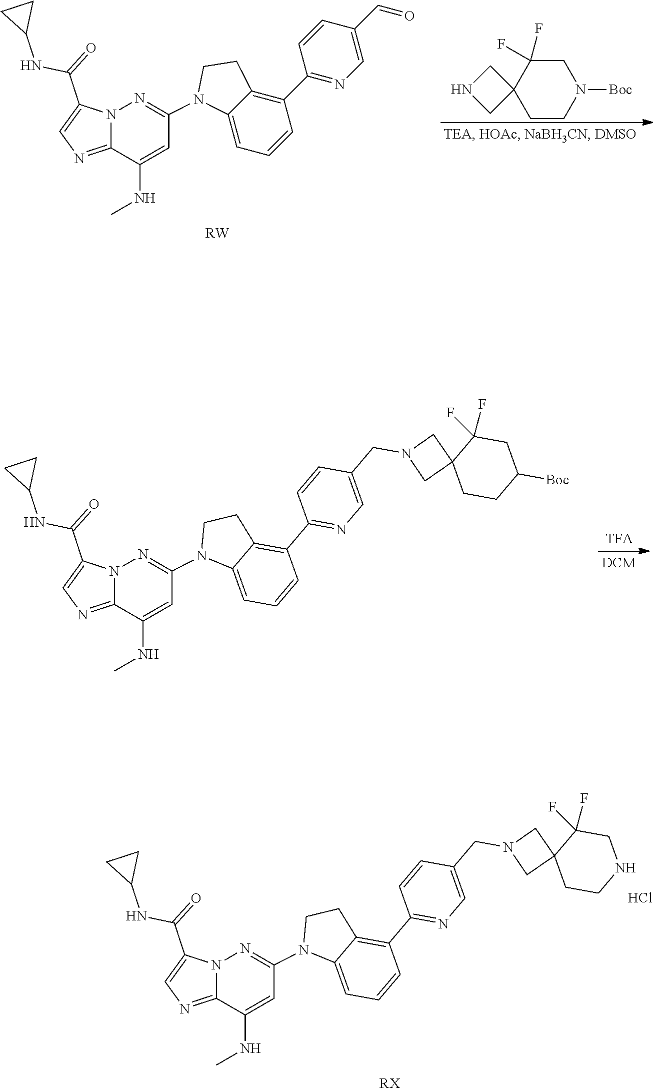

Step 1—Tert-butyl 2-[(6-{1-[3-(cyclopropylcarbamoyl)-8-(methylamino)imidazo[1,2-b]pyridazin-6-yl]-2,3-dihydroindol-4-yl}pyridin-3-yl)methyl]-5,5-difluoro-2,7-diazaspiro[3.5]nonane-7-carboxylate. To a stirred solution of N-cyclopropyl-6-[4-(5-formylpyridin-2-yl)-2,3-dihydroindol-1-yl]-8-(methylamino)imidazo[1,2-b]pyridazine-3-carboxamide (248 mg, 0.547 mmol, Intermediate RW) and Et₃N (166 mg, 1.641 mmol) in DMSO (5 mL) were added tert-butyl 5,5-difluoro-2,7-diazaspiro[3.5]nonane-7-carboxylate (201 mg, 0.766 mmol) and AcOH (164 mg, 2.735 mmol) at rt. The resulting mixture was stirred for 1 h at 50° C. under nitrogen atmosphere. To the above mixture was added NaBH₃CN (103 mg, 1.641 mmol) at rt. The resulting mixture was stirred for an additional 2 h at 50° C. On completion, the mixture was purified directly by reversed-phase flash chromatography (column, C18 silica gel; mobile phase, MeCN in Water (10 mmol/L NH₄HCO₃), 20% to 60% gradient in 30 min; detector, UV 254 nm) to give the title compound (80 mg, 21% yield) as a brown yellow solid. LC/MS (ESI, m/z): [(M+H)]⁺=700.4.

Step 2—N-cyclopropyl-6-(4-(5-((5,5-difluoro-2,7-diazaspiro[3.5]nonan-2-yl)methyl)pyridin-2-yl)indolin-1-yl)-8-(methylamino)imidazo[1,2-b]pyridazine-3-carboxamide trifluoroacetate. To a stirred solution of tert-butyl 2-[(6-{1-[3-(cyclopropylcarbamoyl)-8-(methylamino)imidazo[1,2-b]pyridazin-6-yl]-2,3-dihydroindol-4-yl}pyridin-3-yl)methyl]-5,5-difluoro-2,7-diazaspiro[3.5]nonane-7-carboxylate (100 mg, 0.143 mmol) in DCM (6 mL) was added TFA (2 mL) at rt and the mixture was stirred at rt for 1 h. On completion, the mixture was concentrated under reduced pressure. The residue was purified by trituration with Et₂O (5 mL) to afford the title compound (80 mg, 88% yield) as a white solid. LC/MS (ESI, m/z): [(M+H)]⁺=600.2.

### N-cyclopropyl-6-[4-(4-formylpyridin-2-yl)-2,3-dihydroindol-1-yl]-8-(methylamino)imidazo[1,2-b]pyridazine-3-carboxamide (Intermediate RY)

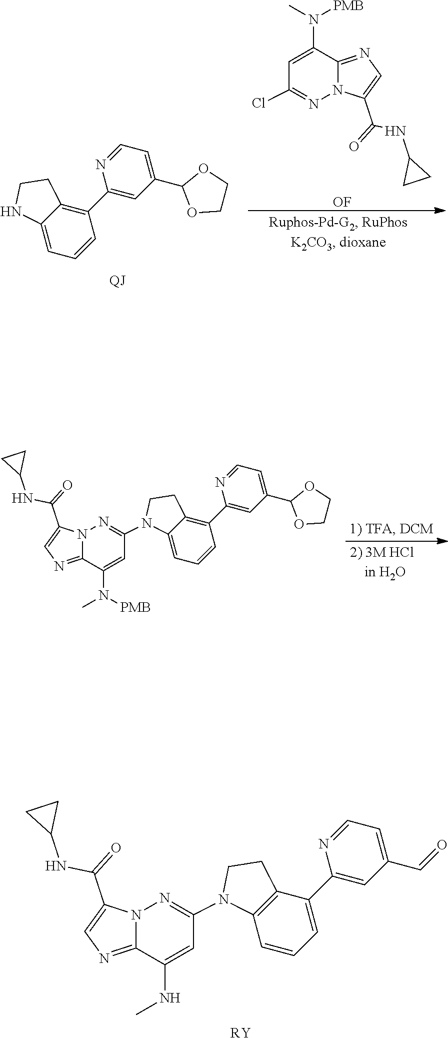

Step 1—N-cyclopropyl-6-{4-[4-(1,3-dioxolan-2-yl)pyridin-2-yl]-2,3-dihydroindol-1-yl}-8-{[(4-methoxyphenyl)methyl](methyl)amino}imidazo[1,2-b]pyridazine-3-carboxamide. To a stirred solution of 4-[4-(1,3-dioxolan-2-yl)pyridin-2-yl]-2,3-dihydro-1H-indole (800 mg, 2.99 mmol, Intermediate Q J) and 6-chloro-N-cyclopropyl-8-{[(4-methoxyphenyl)methyl](methyl)amino}imidazo[1,2-b]pyridazine-3-carboxamide (842.61 mg, 2.184 mmol. Intermediate OF) in toluene (10 mL) were added RuPhos (101.90 mg, 0.218 mmol), RuPhos-PdCl-2nd G (169.84 mg, 0.218 mmol) and LiHMDS (13.10 mL, 13.10 mmol) at rt under nitrogen atmosphere. The mixture was purged with nitrogen 3 times and was then stirred for 2 h at 80° C. under nitrogen atmosphere. On completion, the mixture was cooled to rt and was acidified to pH 5 with FA. The resulting mixture was concentrated under reduced pressure. The residue was purified by silica gel column chromatography, eluted with PE/EA (EA), to afford the title compound (700 mg, 52% yield) as a yellow solid. LC/MS (ESI, m/z): [(M+H)]⁺=618.3.

Step 2—N-cyclopropyl-6-[4-(4-formylpyridin-2-yl)-2,3-dihydroindol-1-yl]-8-(methylamino)imidazo[1,2-b]pyridazine-3-carboxamide. To a solution N-cyclopropyl-6-{4-[4-(1,3-dioxolan-2-yl)pyridin-2-yl]-2,3-dihydroindol-1-yl}-8-{[(4-methoxyphenyl)methyl](methyl)amino}imidazo[1,2-b]pyridazine-3-carboxamide (700 mg, 1.14 mmol) in DCM (10 mL) was added TFA (3 mL) dropwise at rt. The resulting solution was stirred for 2 h at rt. On completion, the reaction was concentrated under reduced pressure. The residue was dissolved in 3 M aq. HCl (10 mL) and stirred for 16 h at 50° C. under nitrogen atmosphere. On completion, the mixture was basified to pH 8 with saturated NaHCO₃ (aq.). The precipitated solids were collected by filtration and washed with EtOAc (3×30 mL). The precipitated solids were dried by lyophilization to afford the title compound (490 mg, 99% yield) as a brown solid. LC/MS (ESI, m/z): [(M+H)]⁺=454.2.

### 6-(4-(5-((2,7-diazaspiro[3.5]nonan-2-yl)methyl)pyridin-2-yl)indolin-1-yl)-N-((1R,2R)-2-methoxycyclobutyl)-8-(methylamino)imidazo[1,2-b]pyridazine-3-carboxamide trifluoroacetate (Intermediate SA)

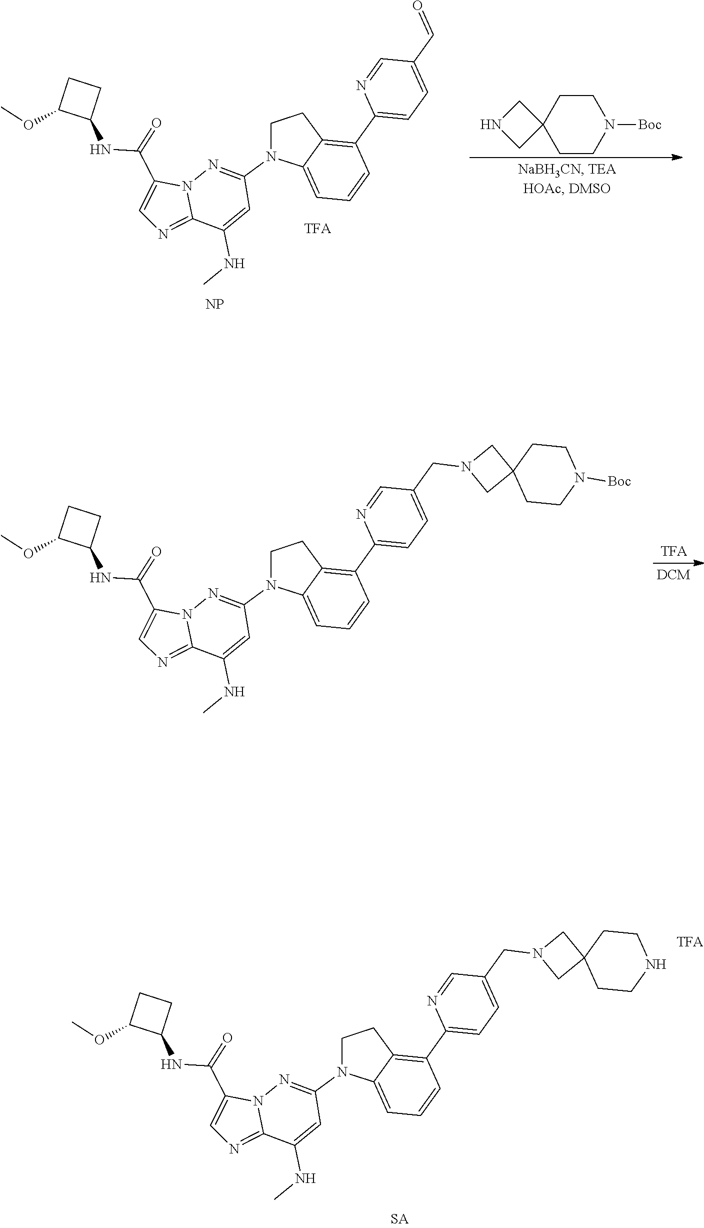

Step 1—Tert-butyl 2-((6-(1-(3-(((1R,2R)-2-methoxycyclobutyl)carbamoyl)-8-(methylamino)imidazo[1,2-b]pyridazin-6-yl)indolin-4-yl)pyridin-3-yl)methyl)-2,7-diazaspiro[3.5]nonane-7-carboxylate. To a stirred mixture of 6-(4-(5-formylpyridin-2-yl)indolin-1-yl)-N-((1R,2R)-2-methoxycyclobutyl)-8-(methylamino)imidazo[1,2-b]pyridazine-3-carboxamide trifluoroacetate (130 mg, 0.261 mmol, Intermediate NP) and Et₃N (53 mg, 0.522 mmol) in DMSO (3 mL) was added tert-butyl 2,7-diazaspiro[3.5]nonane-7-carboxylate (59 mg, 0.261 mmol) and HOAc (157 mg, 2.610 mmol) at rt. The resulting mixture was stirred for 1 h at rt. To the above mixture was added NaBH₃CN (82 mg, 1.305 mmol) in portions at rt. The resulting mixture was stirred for an additional 2 h at 50° C. On completion, the mixture was cooled to rt and purified directly by reversed-phase flash chromatography (Column: Spherical C18, 20˜40 μm, 120 g; Mobile Phase A: Water (plus 10 mM NH₄HCO₃), Mobile Phase B: ACN; Flow rate: 50 mL/min; Gradient (B %): 5%-40%, 4 min; 40%˜60%, 20 min; 60%˜95%; 2 min; 95%, 5 min; Detector: 254 nm; the fractions containing desired product were collected at 59% B and concentrated under reduced pressure) to afford the title compound (120 mg, 64% yield) as a white solid. LC/MS (ESI, m/z): [(M+1)]⁺=708.4.

Step 2—6-(4-(5-((2,7-Diazaspiro[3.5]nonan-2-yl)methyl)pyridin-2-yl)indolin-1-yl)-N-((1R,2R)-2-methoxycyclobutyl)-8-(methylamino)imidazo[1,2-b]pyridazine-3-carboxamide trifluoroacetate. A mixture of tert-butyl 2-((6-(1-(3-(((1R,2R)-2-methoxycyclobutyl)carbamoyl)-8-(methylamino)imidazo[1,2-b]pyridazin-6-yl)indolin-4-yl)pyridin-3-yl)methyl)-2,7-diazaspiro[3.5]nonane-7-carboxylate (100 mg, 0.141 mmol) in DCM (1.5 mL) and TFA (0.5 mL) was stirred for 1 h at rt. On completion, the mixture was concentrated under reduced pressure. The residue was purified by trituration with Et₂O (5 mL) to afford the title compound (80 mg, 93% yield) as a brown solid. LC/MS (ESI, m/z): [(M+1)]⁺=608.4.

### 2-(1-(2,6-Dioxopiperidin-3-yl)-3-methyl-2-oxo-2,3-dihydro-1H-benzo[d]imidazol-5-yl)acetaldehyde (Intermediate SB)

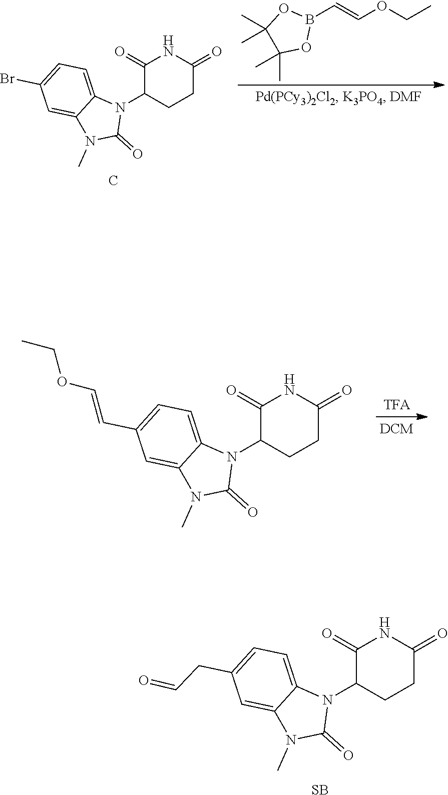

Step 1—(E)-3-(5-(2-ethoxyvinyl)-3-methyl-2-oxo-2,3-dihydro-TH-benzo[d]imidazol-1-yl)piperidine-2,6-dione. To a stirred mixture of 3-(5-bromo-3-methyl-2-oxo-2,3-dihydro-1H-benzo[d]imidazol-1-yl)piperidine-2,6-dione (5.00 g, 14.8 mmol, Intermediate C), (E)-2-(2-ethoxyvinyl)-4,4,5,5-tetramethyl-1,3,2-dioxaborolane (3.51 g, 17.7 mmol) and K₃PO₄ (9.42 g, 44.4 mmol) in DMF (50 mL) was added Pd(PCy₃)₂Cl₂ (1.12 g, 1.48 mmol) at rt under nitrogen atmosphere. The resulting mixture was stirred overnight at 90° C. under nitrogen atmosphere. On completion, the mixture was cooled to rt and purified directly by reversed-phase flash chromatography (Column: Spherical C18, 20˜40 μm, 330 g; Mobile Phase A: Water (plus 10 mM NH₄HCO₃), Mobile Phase B: ACN; Flow rate: 80 mL/min; Gradient (B %): 5%˜40%, 4 min; 40%-60%, 20 min; 60%-95%; 2 min; 95%, 5 min; Detector: 254 nm; the fractions containing desired product were collected at 48% B and concentrated under reduced pressure) to afford the title compound (3.2 g, 65% yield) as a brown yellow solid. LC/MS (ESI, m/z): [(M+1)]=330.2.

Step 2—2-(1-(2,6-Dioxopiperidin-3-yl)-3-methyl-2-oxo-2,3-dihydro-1H-benzo[d]imidazol-5-yl)acetaldehyde. A solution of (E)-3-(5-(2-ethoxyvinyl)-3-methyl-2-oxo-2,3-dihydro-1H-benzo[d]imidazol-1-yl)piperidine-2,6-dione (200 mg, 0.607 mmol) in TFA (0.5 mL) and DCM (2.5 mL) was stirred for 1 h at rt. On completion, the mixture was basified to pH 7 with saturated NaHCO₃ (aq.) and the mixture was concentrated under reduced pressure. The resulting mixture was purified by reversed-phase flash chromatography (Column: Spherical C18, 20-40 μm, 120 g; Mobile Phase A: Water (plus 10 mM NH₄HCO₃), Mobile Phase B: ACN; Flow rate: 50 mL/min; Gradient (B %): 5%30%, 4 min; 30%˜40%, 20 min; 40%-95%; 2 min; 95%, 5 min; Detector: 254 nm; the fractions containing desired product were collected at 52% B) and concentrated under reduced pressure to afford the title compound (120 mg, 39% yield) as a red solid. LC/MS (ESI, m/z): [(M+1)]⁺=302.3.

### 3-(4-Bromo-2-chlorophenyl)piperidine-2,6-dione (Intermediate SC)

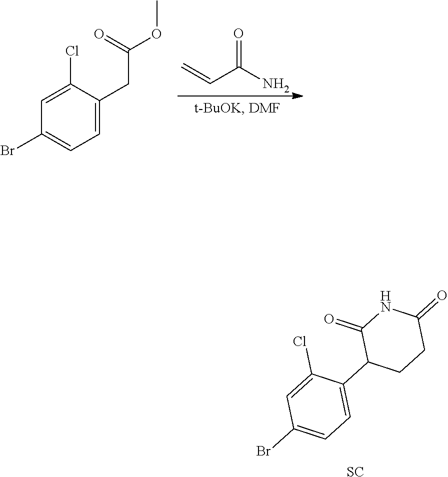

To a stirred mixture of methyl 2-(4-bromo-2-chlorophenyl)acetate (10 g, 40 mmol) and polyacrylamide (2.70 g, 37.9 mmol) in DMF (15 mL) was added t-BuOK (6.39 g, 56.9 mmol) at rt under nitrogen atmosphere. The resulting mixture was stirred for 2 h at rt under nitrogen atmosphere. The resulting mixture was concentrated under reduced pressure. The resulting mixture was extracted with EtOAc (2×200 mL). The combined organic layers were washed with brine (3×200 mL), and dried over anhydrous Na₂SO₄. After filtration, the filtrate was concentrated under reduced pressure. The residue was purified by silica gel column chromatography, eluted with DCM/EA (1:1) to afford crude product. The crude was further purified by reverse flash chromatography (column, C18 silica gel; mobile phase A: water (10 mmol/L NH₄HCO₃), mobile phase B: ACN, 30% to 50% gradient in 30 min; detector, UV 254 nm; the fractions were collected at 33% and concentrated under reduced pressure) to afford the title compound (6 g, 52% yield) as a white solid. LC/MS (ESI, m/z): [(M+H)]⁺=302.0, 304.0.

### 3-Chloro-4-(2,6-dioxopiperidin-3-yl)benzaldehyde (Intermediate SD)

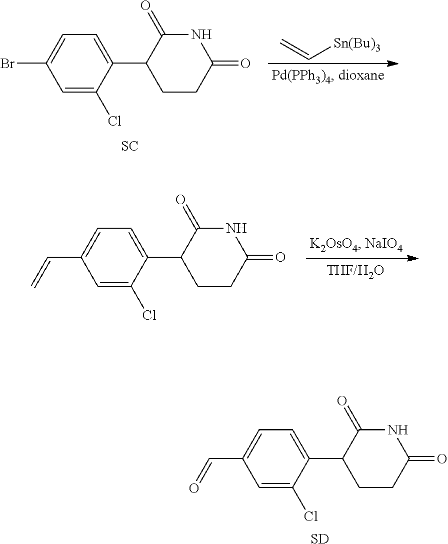

Step 1—3-(2-Chloro-4-ethenylphenyl)piperidine-2,6-dione. To a stirred mixture of 3-(4-bromo-2-chlorophenyl)piperidine-2,6-dione (2 g, 7 mmol. Intermediate SC) and tributyl(ethenyl)stannane (3.14 g, 9.92 mmol) in dioxane (30 mL) was added Pd(PPh₃)₂Cl₂ (0.46 g, 0.66 mmol) in portions at rt under air atmosphere. The resulting mixture was stirred for 1 h at 120° C. under nitrogen atmosphere. On completion, the mixture was cooled to rt and diluted with water (250 mL). The resulting mixture was extracted with EtOAc (3×250 mL). The combined organic layers were washed with water (2×250 mL), and dried over anhydrous Na₂SO₄. After filtration, the filtrate was concentrated under reduced pressure. The residue was purified by reverse flash chromatography (column, WelFlash™ C18-I, 20-40 μm, 330 g; Eluent A: PE; Eluent B: EtOAc, 0% to 50% gradient in 30 min; Flow rate: 80 mL/min; detector: UV 254/280 nm, desired fractions were collected at 20% B and concentrated under reduced pressure) to afford the title compound (1.6 g, 90% yield) as a yellow solid. LC/MS (ESI, m/z): [(M+1)]⁺=250.1.

Step 2—3-Chloro-4-(2,6-dioxopiperidin-3-yl)benzaldehyde. To a stirred mixture of 3-(2-chloro-4-ethenylphenyl)piperidine-2,6-dione (800 mg, 3.20 mmol) and NaIO₄ (3426.49 mg, 16.020 mmol) in THF (10 mL) and water (5 mL) was added K₂OsO₄·2H₂O (118.05 mg, 0.320 mmol) dropwise at 0° C. under N₂ atmosphere. The resulting mixture was stirred for 1 h at rt under nitrogen atmosphere. On completion, the mixture was diluted with water (200 mL) and extracted with EtOAc (3×200 mL). The combined organic layers were washed with water (2×200 mL), and dried over anhydrous Na₂SO₄. After filtration, the filtrate was concentrated under reduced pressure. The residue was purified by reverse flash chromatography (column, WelFlash™ C18-I, 20-40 μm, 330 g; Eluent A: PE; Eluent B: EtOAc, 30% to 90% gradient in 30 min; Flow rate: 80 mL/min; detector: UV 254/280 nm, desired fractions were collected at 78% B and concentrated under reduced pressure) to give the title compound (550 mg, 68% yield) as a yellow solid. LC/MS (ESI, m/z): [(M+1)]⁺=252.1.

### Tert-butyl 2-(6-chloropyridin-3-yl)-2,7-diazaspiro[3.5]nonane-7-carboxylate (Intermediate SE)

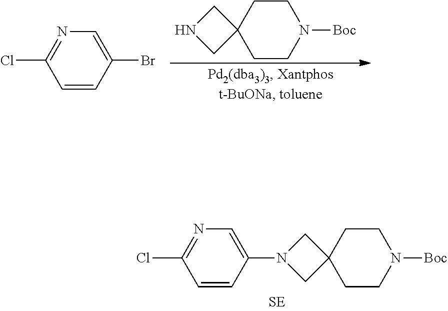 [SMILES: Clc1ccc(Br)cn1 ; CC(C)(C)OC(=O)N1CCC2(CC1)CNC2 ; CC(C)(C)OC(=O)N1CCC2(CC1)CN(c1ccc(Cl)nc1)C2]

To a stirred solution of 5-bromo-2-chloropyridine (3.00 g, 15.6 mmol, CAS #53939-30-3) and tert-butyl 2,7-diazaspiro[3.5]nonane-7-carboxylate (4.23 g, 18.7 mmol, CAS #1023301-84-9) in toluene (30 mL) was added XantPhos (1.80 g, 3.12 mmol) and t-BuONa (4.49 g, 46.8 mmol) at rt under nitrogen atmosphere. To the above mixture was added Pd₂(dba)₃ (1.43 g, 1.56 mmol) at rt under nitrogen atmosphere and the mixture was stirred for 16 h at 90° C. under nitrogen atmosphere. On completion, the mixture was cooled to rt and concentrated under reduced pressure. The crude product was purified by silica gel column chromatography (Mobile Phase A: Petroleum ether, Mobile Phase B: EtOAc; Gradient: 30% B to 50% B in 20 min, 254 nm; the fractions containing the desired product were collected at 45% B) to afford the title compound (2.00 g, 38% yield) as a light yellow solid. LC/MS (ESI, m/z): [(M+H)]⁺=338.2.

### N-((1R,2S)-2-fluorocyclopropyl)-8-((4-methoxybenzyl)(methyl)amino)-6-(4-(4,4,5,5-tetramethyl-1,3,2-dioxaborolan-2-yl)indolin-1-yl)imidazo[1,2-b]pyridazine-3-carboxamide (Intermediate SF)

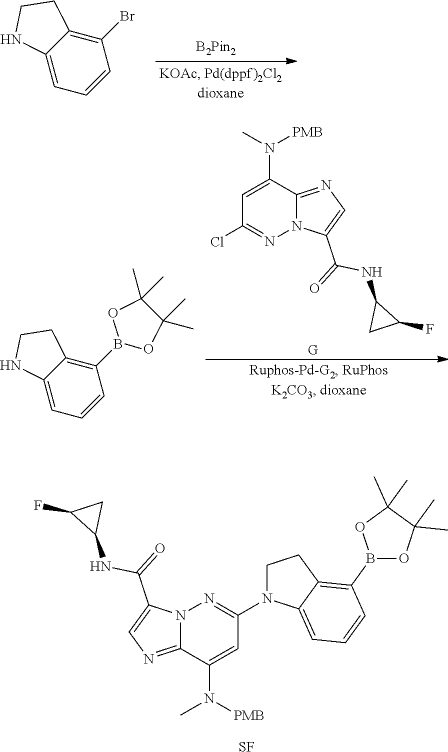

Step 1—4-(4,4,5,5-Tetramethyl-1,3,2-dioxaborolan-2-yl)-2,3-dihydro-1H-indole. To a stirred mixture of 4-bromo-2,3-dihydro-1H-indole (10 g, 50 mmol), 4,4,5,5-tetramethyl-2-(4,4,5,5-tetramethyl-1,3,2-dioxaborolan-2-yl)-1,3,2-dioxaborolane (25.64 g, 101.0 mmol) and KOAc (14.87 g, 151.5 mmol) in dioxane (100 mL) was added Pd(dppf)Cl₂·CH₂Cl₂ (4.11 g, 5.05 mmol) in portions at rt under nitrogen atmosphere. The resulting mixture was stirred for 2 h at 90° C. under nitrogen atmosphere. On completion, the mixture was concentrated under reduced pressure. The residue was purified by silica gel column chromatography, eluted with PE/EA (5:1) to afford the title compound (9.50 g, 77% yield) as a brown oil. LC/MS (ESI, m/z): [(M+1)]⁺=246.2.

Step 2—N-[(1R,2S)-2-fluorocyclopropyl]-8-{[(4-methoxyphenyl)methyl](methyl)amino}-6-[4-(4,4,5,5-tetramethyl-1,3,2-dioxaborolan-2-yl)-2,3-dihydroindol-1-yl]imidazo[1,2-b]pyridazine-3-carboxamide. To a stirred mixture of 6-chloro-N-[(1R,2S)-2-fluorocyclopropyl]-8-{[(4-methoxyphenyl)methyl](methyl)amino}imidazo[1,2-b]pyridazine-3-carboxamide (7.5 g, 19 mmol, Intermediate G) and 4-(4,4,5,5-tetramethyl-1,3,2-dioxaborolan-2-yl)-2,3-dihydro-1H-indole (6.83 g, 27.9 mmol) in dioxane (150 mL) were added RuPhos (1.73 g, 3.71 mmol), RuPhos-PdCl-2nd G (1.44 g, 1.86 mmol) and K₂CO₃ (5.13 g, 37.1 mmol) in portions at rt under N₂ atmosphere. The resulting mixture was stirred for 16 h at 90° C. under nitrogen atmosphere. On completion, the mixture was cooled to rt and concentrated under reduced pressure. The residue was purified by silica gel column chromatography, eluted with PE/EA (1:1) to afford the title compound (5.9 g, 52% yield) as a yellow solid. LC/MS (ESI, m/z): [(M+1)]⁺=613.3.

### 6-(4-(5-(2,7-diazaspiro[3.5]nonan-2-yl)pyridin-2-yl)indolin-1-yl)-N-((1R,2S)-2-fluorocyclopropyl)-8-(methylamino)imidazo[1,2-b]pyridazine-3-carboxamide trifluoroacetate (Intermediate SG)

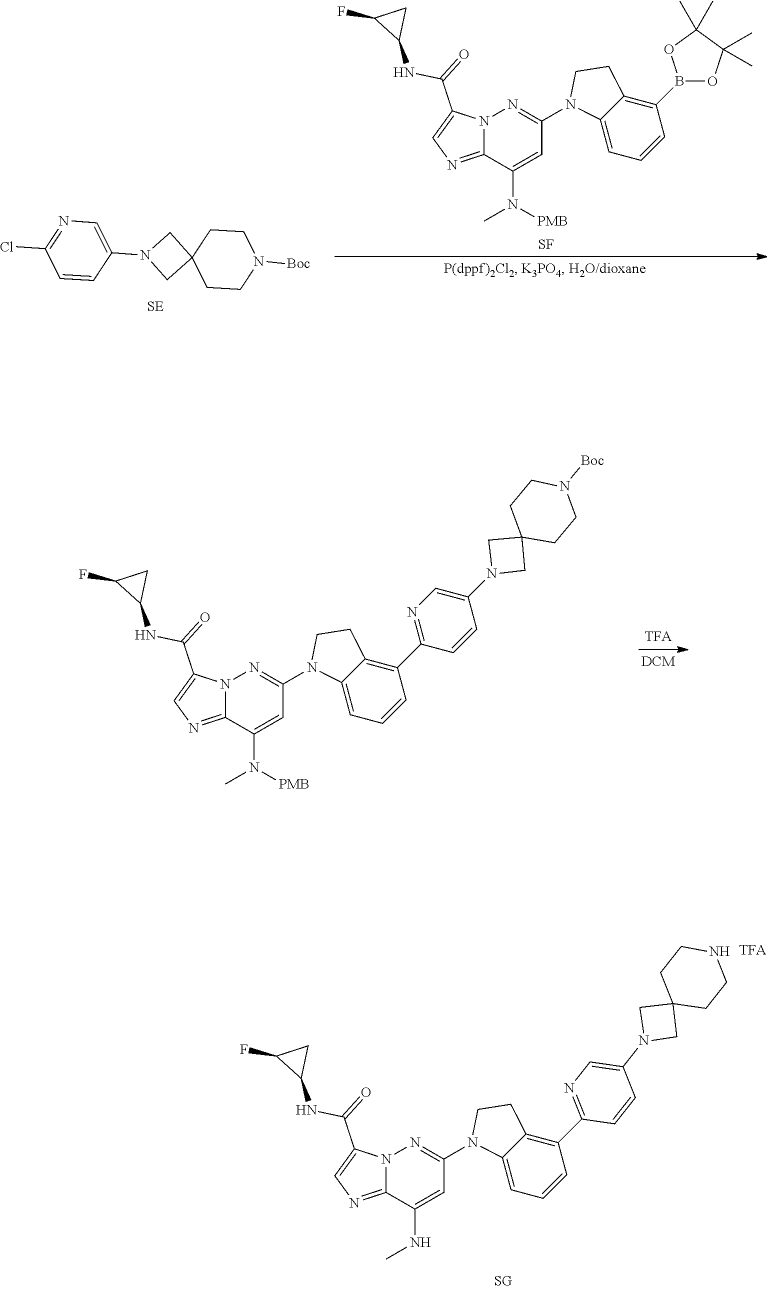

Step 1—Tert-butyl 2-(6-(1-(3-(((1R,2S)-2-fluorocyclopropyl)carbamoyl)-8-((4-methoxybenzyl)(methyl)amino)imidazo[1,2-b]pyridazin-6-yl)indolin-4-yl)pyridin-3-yl)-2,7-diazaspiro[3.5]nonane-7-carboxylate. To a stirred solution of tert-butyl 2-(6-chloropyridin-3-yl)-2,7-diazaspiro[3.5]nonane-7-carboxylate (800 mg, 2.37 mmol, Intermediate SE) and N-((1R,2S)-2-fluorocyclopropyl)-8-((4-methoxybenzyl)(methyl)amino)-6-(4-(4,4,5,5-tetramethyl-1,3,2-dioxaborolan-2-yl)indolin-1-yl)imidazo[1,2-b]pyridazine-3-carboxamide (1.45 g, 2.37 mmol, Intermediate SF) and K₃PO₄ (1.51 g, 7.10 mmol) in H₂O (2 mL) and dioxane (10 mL) was added Pd(DtBPF)Cl₂(309 mg, 0.474 mmol) in turns at rt under nitrogen atmosphere. The resulting mixture was stirred for 16 h at 90° C. under nitrogen atmosphere. On completion, the mixture was concentrated under reduced pressure. The mixture was purified by silica gel column chromatography (Mobile Phase A: PE, Mobile Phase B: EA; Gradient: 50% to 100% B in 25 min, 254 nm; the fractions containing the desired product were collected at 74% B and concentrated under reduced pressure) to afford the title compound (450 mg, 24% yield) as a yellow oil. LC/MS (ESI, m/z): [(M+H)]⁺=788.5.

Step 2—6-(4-(5-(2,7-Diazaspiro[3.5]nonan-2-yl)pyridin-2-yl)indolin-1-yl)-N-((1R,2S)-2-fluorocyclopropyl)-8-(methylamino)imidazo[1,2-b]pyridazine-3-carboxamide trifluoroacetate. To a stirred solution of tert-butyl 2-(6-(1-(3-(((1R,2S)-2-fluorocyclopropyl)carbamoyl)-8-((4-methoxybenzyl)(methyl)amino)imidazo[1,2-b]pyridazin-6-yl)indolin-4-yl)pyridin-3-yl)-2,7-diazaspiro[3.5]nonane-7-carboxylate (450 mg, 0.609 mmol) in DCM (14 mL) was added TFA (7 mL) at rt and the mixture was stirred for 1 h at rt under nitrogen atmosphere. On completion, the mixture was concentrated under reduced pressure. The residue was purified by trituration with Et₂O (60 mL) and dried to give the title compound (540 mg, 94% yield) as a yellow solid. LC/MS (ESI, m/z): [(M+H)]⁺=568.5.

### 3-Chloro-4-(2,6-dioxopiperidin-3-yl)benzoic acid (Intermediate SH)

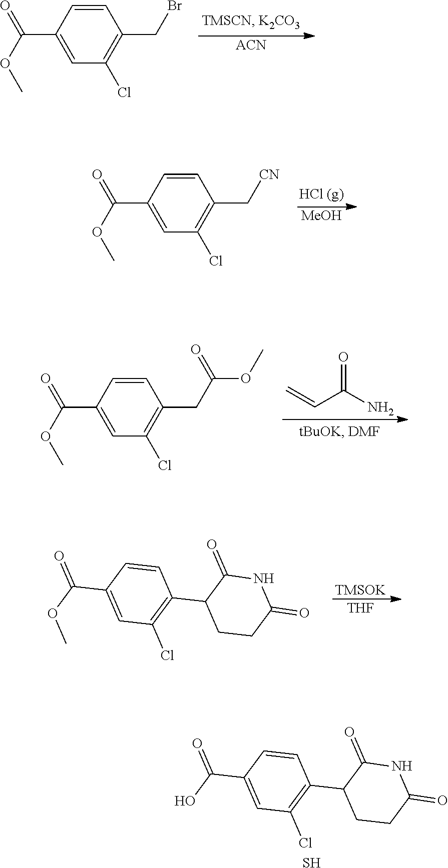

Step 1—Methyl 3-chloro-4-(cyanomethyl)benzoate. To a mixture of methyl 4-(bromomethyl)-3-chlorobenzoate (5 g, 20 mmol), TMSCN (2.82 g, 28.5 mmol) in ACN (250 mL) was added K₂CO₃ (3.93 g, 28.5 mmol) and the mixture was stirred for 16 h at 80° C. On completion, the mixture was cooled to rt and concentrated under reduced pressure. The resulting mixture was diluted with water (500 mL) and extracted with CH₂Cl₂ (3×300 mL). The combined organic layers were washed with brine (3×100 mL), and dried over anhydrous Na₂SO₄. After filtration, the filtrate was concentrated under reduced pressure to afford the title compound (3 g, 75% yield) as a white semi-solid. LC/MS (ESI, m/z): [(M+1)]⁺=210.1.

Step 2—Methyl 3-chloro-4-(2-methoxy-2-oxoethyl)benzoate. To a solution of methyl 3-chloro-4-(cyanomethyl)benzoate (3 g, 14 mmol) in MeOH (150 mL) was bubbled with HCl (g) for 2 h at 10° C. The resulting mixture was stirred 16 h at rt. On completion, the mixture was concentrated under reduced pressure. The resulting mixture was diluted with water (100 mL) and extracted with EtOAc (3×100 mL). The combined organic layers were washed with brine (3×50 mL), and dried over anhydrous Na₂SO₄. After filtration, the filtrate was concentrated under reduced pressure to afford the title compound (1.8 g, 52% yield) as a yellow solid. LC/MS (ESI, m/z): [(M+1)]=243.1.

Step 3—Methyl 3-chloro-4-(2,6-dioxopiperidin-3-yl)benzoate. To a stirred mixture of methyl 3-chloro-4-(2-methoxy-2-oxoethyl)benzoate (1.8 g, 7.4 mmol) and polyacrylamide (632.70 mg, 8.902 mmol) in DMF (18 mL) was added t-BuOK (1.25 g, 11.1 mmol) in portions at rt. The resulting mixture was stirred for 1 h at rt. The resulting mixture was purified by reversed-phase flash chromatography (Column: Spherical C18, 20-40 μm, 330 g; Mobile Phase A: Water (plus 10 mM NH₄HCO₃), Mobile Phase B: ACN; Flow rate: 80 mL/min; Gradient (B %): 5%˜25%, 4 min; 25%˜40%, 20 min; 40%˜95%; 2 min; 95%, 5 min; Detector: 254 nm; the fractions containing desired product were collected at 35% B and concentrated under reduced pressure) to afford the title compound (860 mg, 41% yield) as a white solid. LC/MS (ESI, m/z): [(M+1)]⁺=282.1.

Step 4—3-Chloro-4-(2,6-dioxopiperidin-3-yl)benzoic acid. To a solution of methyl 3-chloro-4-(2,6-dioxopiperidin-3-yl)benzoate (860 mg, 3.05 mmol) in THF (17 mL) was added trimethyl(potassiooxy)silane (1.96 g, 15.3 mmol) in portions. The resulting mixture was stirred for 1 h at rt. On completion, the mixture was purified by reversed-phase flash chromatography (Column: Spherical C18, 20˜40 μm, 330 g; Mobile Phase A: Water (plus 0.1% HCOOH), Mobile Phase B: ACN; Flow rate: 80 mL/min; Gradient (B %): 5%˜20%, 4 min; 20%˜30%, 20 min; 30%˜95%; 2 min; 95%, 5 min; Detector: 254 nm; the fractions containing desired product were collected at 27% B and concentrated under reduced pressure) to afford the title compound (590 mg, 72% yield) as a white solid. LC/MS (ESI, m/z): [(M+1)]⁺=268.1.

### 3-(2,4-Dioxotetrahydropyrimidin-1(2H)-yl)-5-fluoro-4-methylbenzoic acid (Intermediate SI)

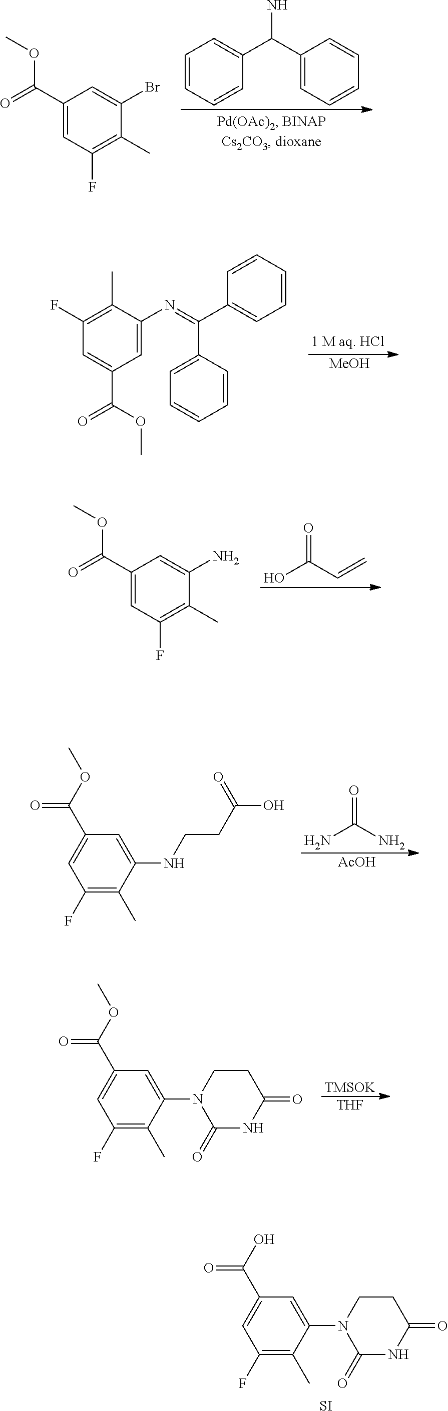

Step 1—Methyl 3-((diphenylmethylene)amino)-5-fluoro-4-methylbenzoate. To a stirred mixture of methyl 3-bromo-5-fluoro-4-methylbenzoate (4.00 g, 16.2 mmol, CAS #1533932-57-8) and diphenylmethanimine (2.93 g, 16.2 mmol) in 1,4-dioxane (40 mL) was added Cs₂CO₃ (15.83 g, 48.57 mmol), BINAP (2.02 g, 3.24 mmol) and Pd(AcO)₂ (0.36 g, 1.62 mmol) at rt under nitrogen atmosphere. The resulting mixture was stirred for 2 h at 100° C. under nitrogen atmosphere. On completion, the solution was cooled to rt and purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 55%-85% B in 25 min; Flow rate: 80 mL/min; Detector: 220/254 nm; desired fractions were collected at 80% B and concentrated under reduced pressure) to give the title compound (5.00 g, 89%) as a brown yellow solid. LC/MS (ESI, m/z): [(M+1)]⁺=348.1.

Step 2—Methyl 3-amino-5-fluoro-4-methylbenzoate. A mixture of methyl 3-((diphenylmethylene)amino)-5-fluoro-4-methylbenzoate (5.00 g, 14.4 mmol) in HCl (25 mL, 1 mmol) and MeOH (25 mL) was stirred for 1 h at 50° C. On completion, the mixture was cooled to rt and extracted with EtOAc (3×100 mL). The combined organic layers were dried over anhydrous Na₂SO₄, filtered, and the filtrate was concentrated under reduced pressure. The residue was purified by silica gel column chromatography, eluted with petroleum ether/EtOAc (3:1), to afford the title compound (2.50 g, 95% yield) as a white solid. LC/MS (ESI, m/z): [(M+1)]⁺=184.1.

Step 3—3-((3-Fluoro-5-(methoxycarbonyl)-2-methylphenyl)amino)propanoic acid. A solution of methyl 3-amino-5-fluoro-4-methylbenzoate (2.50 g, 13.7 mmol) in acrylic acid (1.97 g, 27.3 mmol) was stirred for 1 h at 100° C. On completion, the mixture was cooled to rt and diluted with water (100 mL). The resulting mixture was extracted with EtOAc (3×100 mL) and the combined organic layers were dried over anhydrous Na₂SO₄. After filtration, the filtrate was concentrated under reduced pressure to give the title compound (3.30 g) as a brown yellow solid. LC/MS (ESI, m/z): [(M+1)]=256.1.

Step 4—Methyl 3-(2,4-dioxotetrahydropyrimidin-1(2H)-yl)-5-fluoro-4-methylbenzoate. A mixture of 3-((3-fluoro-5-(methoxycarbonyl)-2-methylphenyl)amino)propanoic acid (3.60 g, 14.1 mmol) and urea (5.93 g, 98.7 mmol) in AcOH (36 mL) was stirred for 16 h at 110° C. On completion, the mixture cooled to rt and poured into ice/water. The precipitated solids were collected by filtration and washed with water (3×50 mL). The solid was dissolved in DMSO (15 mL) and purified by reverse phase flash chromatography (Column: WelFlash™ C18-I, 20-40 μm, 330 g; Eluent A: Water (plus 10 mmol/L NH₄HCO₃); Eluent B: ACN; Gradient: 30%-65% B in 25 min; Flow rate: 80 mL/min; Detector: 220/254 nm; desired fractions were collected at 45% B and concentrated under reduced pressure) to give the title compound (2.50 g, 63% yield) as a white solid. LC/MS (ESI, m/z): [(M+1)]⁺=281.1.

Step 5—3-(2,4-Dioxotetrahydropyrimidin-1(2H)-yl)-5-fluoro-4-methylbenzoic acid. A mixture of methyl 3-(2,4-dioxotetrahydropyrimidin-1(2H)-yl)-5-fluoro-4-methylbenzoate (2.40 g, 8.56 mmol) and trimethyl(potassiooxy)silane (5.47 g, 42.6 mmol) in THF (24 mL) was stirred for 1 h at rt. On completion, the mixture was poured 2 N HCl slowly. The precipitated solids were collected by filtration and washed with water (3×20 mL). Then the solid was dried under vacuum to afford the title compound (1.60 g, 70% yield) as a white solid. LC/MS (ESI, m/z): [(M+1)]⁺=267.1.

### 6-(6-Fluoro-4-(5-formylpyridin-2-yl)indolin-1-yl)-N-((1R,2S)-2-fluorocyclopropyl)-8-(methylamino)imidazo[1,2-b]pyridazine-3-carboxamide trifluoroacetate (Intermediate SJ)

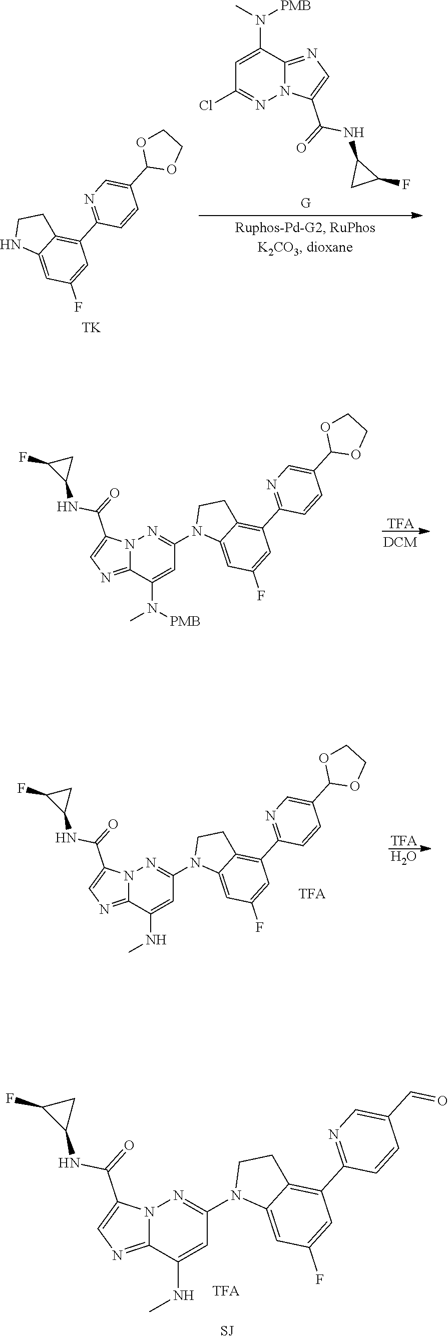

Step 1—6-(4-(5-(1,3-Dioxolan-2-yl)pyridin-2-yl)-6-fluoroindolin-1-yl)-N-((1R,2S)-2-fluorocyclopropyl)-8-((4-methoxybenzyl)(methyl)amino)imidazo[1,2-b]pyridazine-3-carboxamide. To a stirred solution of 6-chloro-N-[(1R,2S)-2-fluorocyclopropyl]-8-{[(4-methoxyphenyl)methyl](methyl)amino}imidazo[1,2-b]pyridazine-3-carboxamide (1.97 g, 4.89 mmol, Intermediate G), K₂CO₃ (2.03 g, 14.7 mmol) and 4-(5-(1,3-dioxolan-2-yl)pyridin-2-yl)-6-fluoroindoline (1.40 g, 4.89 mmol, Intermediate TK) in 1,4-dioxane (30 mL) were added RuPhos (0.46 g, 0.98 mmol) and RuPhos-PdCl-2nd G (0.38 g, 0.48 mmol) at rt under nitrogen atmosphere. The resulting mixture was stirred for 3 h at 100° C. under nitrogen atmosphere. On completion, the mixture was cooled to rt and purified directly by silica gel column chromatography, eluted with petroleum ether/EtOAc (1:9), to afford the title compound (1.4 g, 43% yield) as a yellow solid. LC/MS (ESI, m/z): [(M+1)]⁺=654.2.

Step 2—6-(4-(5-(1,3-Dioxolan-2-yl)pyridin-2-yl)-6-fluoroindolin-1-yl)-N-((1R,2S)-2-fluorocyclopropyl)-8-(methylamino)imidazo[1,2-b]pyridazine-3-carboxamide 2,2,2-trifluoroacetate. To a stirred solution of 6-(4-(5-(1,3-dioxolan-2-yl)pyridin-2-yl)-6-fluoroindolin-1-yl)-N-((1R,2S)-2-fluorocyclopropyl)-8-((4-methoxybenzyl)(methyl)amino)imidazo[1,2-b]pyridazine-3-carboxamide (1.40 g, 2.14 mmol) in DCM (30 mL) was added TFA (10 mL) at rt and the mixture was stirred for 1 h at rt. On completion, the mixture was concentrated under vacuum to afford the title compound (1.2 g, TFA salt) as a brown oil. LC/MS (ESI, m/z): [(M+1)]⁺=534.2.

Step 3—6-(6-Fluoro-4-(5-formylpyridin-2-yl)indolin-1-yl)-N-((1R,2S)-2-fluorocyclopropyl)-8-(methylamino)imidazo[1,2-b]pyridazine-3-carboxamide 2,2,2-trifluoroacetate. To a stirred solution of 6-(4-(5-(1,3-dioxolan-2-yl)pyridin-2-yl)-6-fluoroindolin-1-yl)-N-((1R,2S)-2-fluorocyclopropyl)-8-(methylamino)imidazo[1,2-b]pyridazine-3-carboxamide 2,2,2-trifluoroacetate (1.20 g, 1.84 mmol) in H₂O (20 mL) and TFA (20 mL) at rt. The resulting mixture was stirred for 1 h at 50° C. On completion, the mixture was cooled to rt and neutralized to pH 7 with NaOH. The precipitated solids were collected by filtration, washed with water (3×20 mL), and dried under vacuum to give the title compound (1 g) as a yellow solid. LC/MS (ESI, m/z): [(M+1)]⁺=490.2.

### 6-(4-(5-((1,1-Difluoro-3,9-diazaspiro[5.5]undecan-3-yl)methyl)pyridin-2-yl)-6-fluoroindolin-1-yl)-N-((1R,2S)-2-fluorocyclopropyl)-8-(methylamino)imidazo[1,2-b]pyridazine-3-carboxamide trifluoroacetate (Intermediate SK)

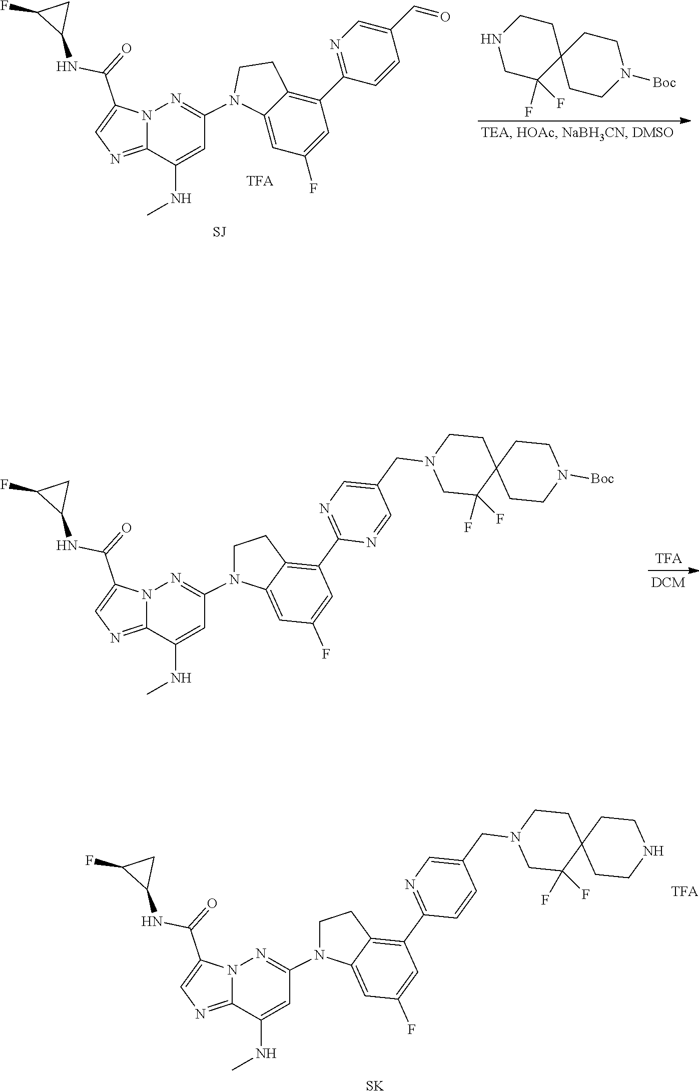

Step 1—Tert-butyl7,7-difluoro-9-((6-(6-fluoro-1-(3-(((1R,2S)-2-fluorocyclopropyl)carbamoyl)-8-(methylamino)imidazo[1,2-b]pyridazin-6-yl)indolin-4-yl)pyridin-3-yl)methyl)-3,9-diazaspiro[5.5]undecane-3-carboxylate. To a stirred solution of tert-butyl 7,7-difluoro-3,9-diazaspiro[5.5]undecane-3-carboxylate (118 mg, 0.409 mmol) and TEA (62 mg, 0.613 mmol) in DMSO (2 mL) were added 6-(6-fluoro-4-(5-formylpyridin-2-yl)indolin-1-yl)-N-((1R,2S)-2-fluorocyclopropyl)-8-(methylamino)imidazo[1,2-b]pyridazine-3-carboxamide trifluoroacetate (200 mg, 0.409 mmol, Intermediate SJ) and HOAc (73 mg, 1.2 mmol) at rt. The resulting mixture was stirred for 2 h at 50° C. Then NaBH₃CN (102 mg, 1.636 mmol) was added in portions at rt. The resulting mixture was stirred for an additional 2 h at 50° C. On completion, the mixture was cooled to rt and purified directly by reverse phase flash chromatography (Column: WelFlash™ C18-I, 20-40 μm, 120 g; Eluent A: Water (plus 10 mmol/L NH₄HCO₃); Eluent B: ACN; Gradient: 30%-60% B in 30 min; Flow rate: 60 mL/min; Detector: 220/254 nm; desired fractions were collected at 47% B and concentrated under reduced pressure) to afford the title compound (150 mg, 48% yield) as a light yellow solid. LC/MS (ESI, m/z): [(M+1)]=764.3.

Step 2—6-(4-(5-((1,1-Difluoro-3,9-diazaspiro[5.5]undecan-3-yl)methyl)pyridin-2-yl)-6-fluoroindolin-1-yl)-N-((1R,2S)-2-fluorocyclopropyl)-8-(methylamino)imidazo[1,2-b]pyridazine-3-carboxamide trifluoroacetate. To a stirred solution of tert-butyl7,7-difluoro-9-((6-(6-fluoro-1-(3-(((1R,2S)-2-fluorocyclopropyl)carbamoyl)-8-(methylamino)imidazo[1,2-b]pyridazin-6-yl)indolin-4-yl)pyridin-3-yl)methyl)-3,9-diazaspiro[5.5]undecane-3-carboxylate (150 mg, 0.196 mmol) in DCM (6 mL) was added TFA (2 mL) at rt and the mixture was stirred for 1 h at rt. The resulting mixture was concentrated under vacuum. The residue was purified by trituration with Et₂O (10 mL) to afford the title compound (120 mg, 92% yield) as a light yellow solid. LC/MS (ESI, m/z): [(M+1)]=664.3.

### 4-(5-(1,3-Dioxolan-2-yl)pyridin-2-yl)indoline (Intermediate SL)

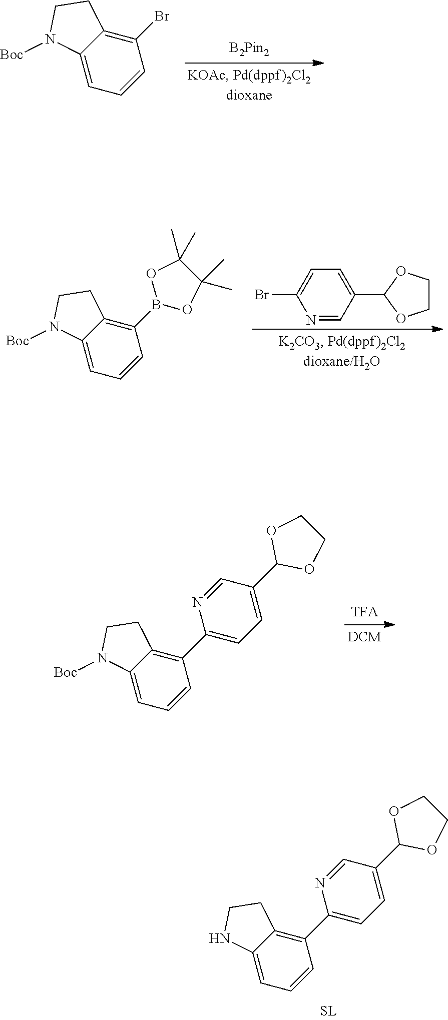

Step 1—Tert-butyl 4-(4,4,5,5-tetramethyl-1,3,2-dioxaborolan-2-yl)indoline-1-carboxylate. To a stirred mixture of tert-butyl 4-bromo-2,3-dihydroindole-1-carboxylate (30.00 g, 100.6 mmol, synthesized via Step 1 of Intermediate OK) and B₂Pin₂ (38.32 g, 150.9 mmol) in dioxane (500 mL) was added KOAc (29.62 g, 301.8 mmol) and Pd(dppf)Cl₂·CH₂Cl₂ (8.20 g, 10.1 mmol) in portions at rt under nitrogen atmosphere. The resulting mixture was stirred for 2 h at 80° C. under nitrogen atmosphere. On completion, the mixture was cooled to rt and purified by silica gel column chromatography, eluted with PE/EA (10:1˜3:1), to afford the title compound (30 g, 86% yield) as a yellow solid. LC/MS (ESI, m/z): [(M+H)]⁺=346.2.

Step 2—Tert-butyl 4-(5-(1,3-dioxolan-2-yl)pyridin-2-yl)indoline-1-carboxylate. To a stirred mixture of tert-butyl 4-(4,4,5,5-tetramethyl-1,3,2-dioxaborolan-2-yl)-2,3-dihydroindole-1-carboxylate (43.80 g, 126.9 mmol) and 2-bromo-5-(1,3-dioxolan-2-yl)pyridine (35.02 g, 152.2 mmol) in dioxane (500 mL) and H₂O (80 mL) were added Pd(dppf)Cl₂CH₂Cl₂ (10.33 g, 12.69 mmol) and K₂CO₃ (35.07 g, 253.7 mmol) in portions at rt under nitrogen atmosphere. The resulting mixture was stirred for 2 h at 80° C. under nitrogen atmosphere. On completion, the mixture was cooled to rt and purified by silica gel column chromatography, eluted with PE/EA (5:1˜3:1), to afford the title compound (20.60 g, 44% yield) as a colorless oil. LC/MS (ESI, m/z): [(M+H)]⁺=369.2.

Step 3—4-(5-(1,3-Dioxolan-2-yl)pyridin-2-yl)indoline. Into a 500 mL round-bottom flask were added tert-butyl 4-[5-(1,3-dioxolan-2-yl)pyridin-2-yl]-2,3-dihydroindole-1-carboxylate (20.00 g, 54.28 mmol) and TFA (100 mL) in DCM (100 mL) at rt. The resulting solution was stirred for 1 h at rt under nitrogen atmosphere. On completion, the mixture was concentrated under reduced pressure. The residue was neutralized to pH 8 with saturated Na₂CO₃ (aq.) and extracted with EtOAc (4×100 mL). The combined organic layers were washed with EtOAc (3×40 mL), and dried over anhydrous Na₂SO₄. After filtration, the filtrate was concentrated under reduced pressure to give the title compound (12.00 g, 82% yield) as a yellow solid. LC/MS (ESI, m/z): [(M+H)]⁺=269.1.

### 6-(4-(5-(1,3-Dioxolan-2-yl)pyridin-2-yl)indolin-1-yl)-8-((4-methoxybenzyl)(methyl)amino)imidazo[1,2-b]pyridazine-3-carboxylic acid (Intermediate SM)

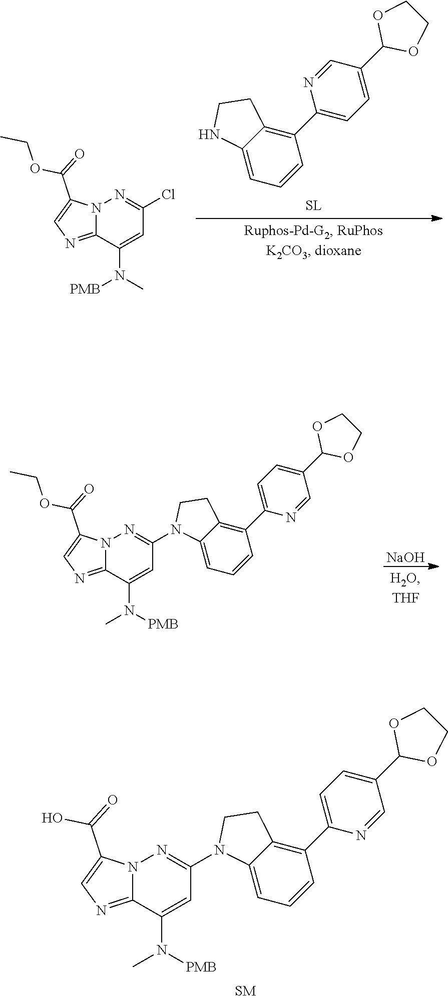

Step 1—Ethyl 6-(4-(5-(1,3-dioxolan-2-yl)pyridin-2-yl)indolin-1-yl)-8-((4-methoxybenzyl)(methyl)amino)imidazo[1,2-b]pyridazine-3-carboxylate. To a stirred solution of ethyl 6-chloro-8-((4-methoxybenzyl)(methyl)amino)imidazo[1,2-b]pyridazine-3-carboxylate (1.50 g, 4.00 mmol, synthesized via Steps 1-2 of Intermediate F) and 4-(5-(1,3-dioxolan-2-yl)pyridin-2-yl)indoline (1.00 g, 4.00 mmol, Intermediate SL) in dioxane (30 mL) were added RuPhos (0.19 g, 0.40 mmol), RuPhos-PdCl-2nd G (0.31 g, 0.40 mmol) and K₂CO₃ (1.66 g, 12.01 mmol) at rt under nitrogen atmosphere. The resulting mixture was then stirred for 2 h at 100° C. under nitrogen atmosphere. On completion, the mixture was cooled to rt and concentrated under reduced pressure. The residue was purified by silica gel column chromatography, eluted with CH₂C2/EtOAc (1:2), to afford the title compound (1.90 g, 78% yield) as a yellow solid. LC/MS (ESI, m/z): [(M+1)]⁺=607.2.

Step 2—6-(4-(5-(1,3-Dioxolan-2-yl)pyridin-2-yl)indolin-1-yl)-8-((4-methoxybenzyl)(methyl)amino)imidazo[1,2-b]pyridazine-3-carboxylic acid. A solution of ethyl 6-(4-(5-(1,3-dioxolan-2-yl)pyridin-2-yl)indolin-1-yl)-8-((4-methoxybenzyl)(methyl)amino)imidazo[1,2-b]pyridazine-3-carboxylate (400 mg, 0.659 mmol) and NaOH (263 mg, 6.59 mmol) in THF (4 mL) and H₂O (4 mL) was stirred for 3 h at 80° C. On completion, the mixture was cooled to rt and concentrated under reduced pressure. The mixture was acidified to pH 5 with conc. HCl. The precipitated solids were collected by filtration and washed with water (3×20 mL) to give the title compound (310 mg, 81% yield) as a yellow solid. LC/MS (ESI, m/z): [(M+1)]⁺=579.2.

### (S)—N-(2,2-difluorocyclopropyl)-6-(4-(5-formylpyridin-2-yl)indolin-1-yl)-8-(methylamino)imidazo[1,2-b]pyridazine-3-carboxamide (Intermediate SN)

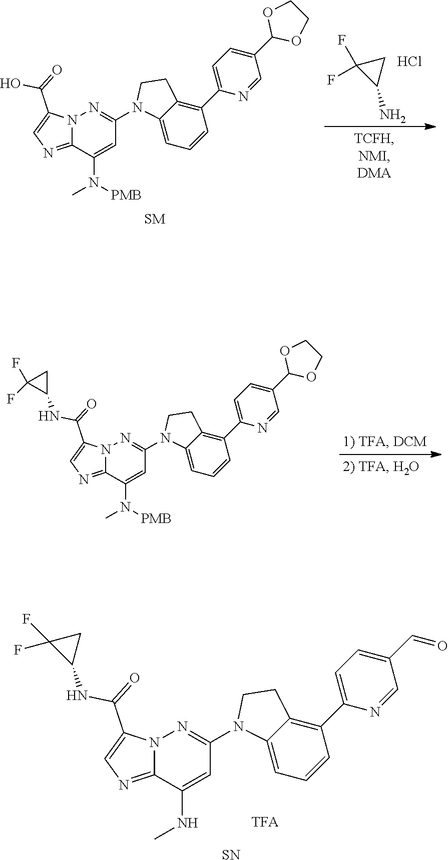

Step 1—(S)-6-(4-(5-(1,3-dioxolan-2-yl)pyridin-2-yl)indolin-1-yl)-N-(2,2-difluorocyclopropyl)-8-((4-methoxybenzyl)(methyl)amino)imidazo[1,2-b]pyridazine-3-carboxamide. To a stirred solution of 6-(4-(5-(1,3-dioxolan-2-yl)pyridin-2-yl)indolin-1-yl)-8-((4-methoxybenzyl)(methyl)amino)imidazo[1,2-b]pyridazine-3-carboxylic acid (400 mg, 0.691 mmol, Intermediate SM) and (S)-2,2-difluorocyclopropan-1-amine hydrochloride (89 mg, 0.691 mmol, CAS #2306252-66-2) in DMA (8 mL) were added TCFH (213 mg, 0.760 mmol, CAS #94790-35-9) and 1-methyl-1H-imidazole (119 mg, 1.45 mmol) at rt. The resulting mixture was stirred for 2 h at 50° C. On completion, the mixture was cooled to rt and purified directly by reversed-phase flash chromatography (Column: Spherical C18, 20˜40 μm, 330 g; Mobile Phase A: Water (plus 10 mM NH₄HCO₃). Mobile Phase B: ACN; Flow rate: 80 mL/min; Gradient (B %): 5%35%, 4 min; 35%˜80%, 20 min; 80%˜95%; 2 min; 95%, 5 min; Detector: 254 nm; the fractions containing desired product were collected at 66% B and concentrated under reduced pressure) to afford the title compound (120 mg, 26% yield) as a yellow solid. LC/MS (ESI, m/z): [(M+1)]⁻=654.3.

Step 2—(S)—N-(2,2-difluorocyclopropyl)-6-(4-(5-formylpyridin-2-yl)indolin-1-yl)-8-(methylamino)imidazo[1,2-b]pyridazine-3-carboxamide. A mixture of (S)-6-(4-(5-(1,3-dioxolan-2-yl)pyridin-2-yl)indolin-1-yl)-N-(2,2-difluorocyclopropyl)-8-((4-methoxybenzyl)(methyl)amino)imidazo[1,2-b]pyridazine-3-carboxamide (120 mg, 0.184 mmol) in TFA (2 mL) and DCM (6 mL) was stirred for 1 h at rt. The resulting mixture was concentrated under reduced pressure. To the above mixture were added H₂O (4 mL) and TFA (4 mL) at it and the mixture was stirred for an additional 2 h at 50° C. On completion, the mixture was cooled to rt and neutralized to pH 8 with saturated Na₂CO₃ (aq.). The precipitated solids were collected by filtration and washed with water (3×10 mL) to afford the title compound (100 mg, TFA salt) as a yellow solid. LC/MS (ESI, m/z): [(M+1)]⁺=490.2.

### (S)-6-(4-(5-((5,5-difluoro-2,7-diazaspiro[3.5]nonan-2-yl)methyl)pyridin-2-yl)indolin-1-yl)-N-(2,2-difluorocyclopropyl)-8-(methylamino)imidazo[1,2-b]pyridazine-3-carboxamide trifluoroacetate (Intermediate SO)

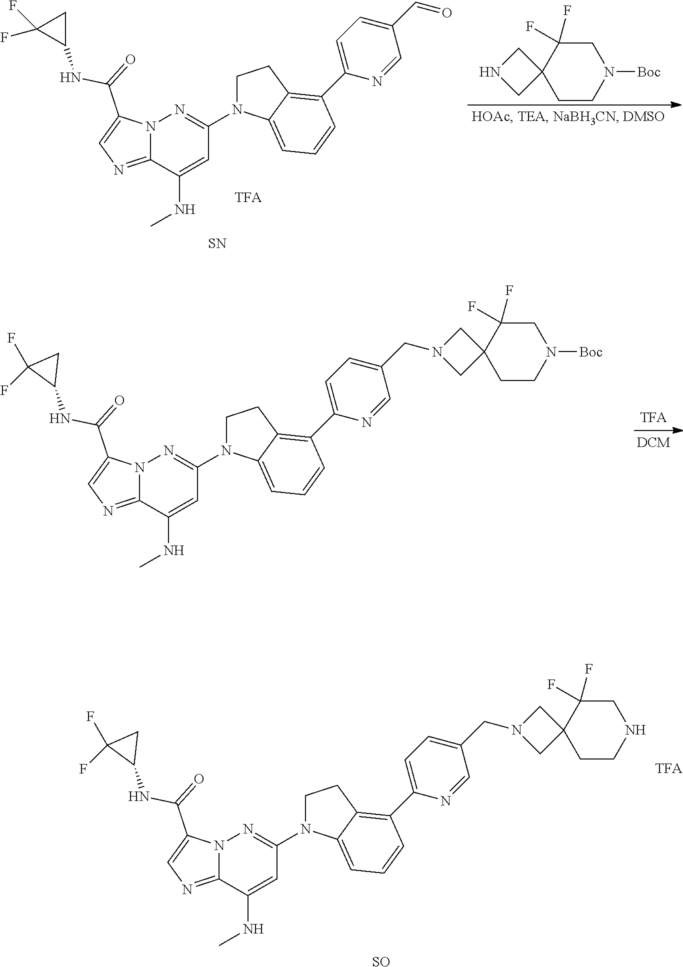

Step 1—Tert-butyl (S)-2-((6-(1-(3-((2,2-difluorocyclopropyl)carbamoyl)-8-(methylamino)imidazo[1,2-b]pyridazin-6-yl)indolin-4-yl)pyridin-3-yl)methyl)-5,5-difluoro-2,7-diazaspiro[3.5]nonane-7-carboxylate. To a stirred solution of (S)—N-(2,2-difluorocyclopropyl)-6-(4-(5-formylpyridin-2-yl)indolin-1-yl)-8-(methylamino)imidazo[1,2-b]pyridazine-3-carboxamide (100 mg, 0,204 mmol, Intermediate SN) and TEA (41 mg, 0.41 mmol) in DMSO (3 mL) were added tert-butyl 5,5-difluoro-2,7-diazaspiro[3.5]nonane-7-carboxylate (53 mg, 0.20 mmol) and AcOH (122 mg, 2.04 mmol) at rt. The resulting mixture was stirred for 1 h at 50° C. To the above mixture was added NaBH₃CN (64 mg, 1.020 mmol) at rt. The resulting mixture was stirred for additional 1 h at 50° C. On completion, the mixture was cooled to rt and purified directly by reversed-phase flash chromatography (Column: Spherical C18, 20˜40 μm, 120 g; Mobile Phase A: Water (plus 10 mM NH₄CO₃), Mobile Phase B: ACN; Flow rate: 50 mL/min; Gradient (B %): 5%˜40%, 4 min; 40%˜70%, 20 min; 70%˜95%; 2 min; 95%, 5 min; Detector: 254 nm; the fractions containing desired product were collected at 66% B and concentrated under reduced pressure) to afford the title compound (60 mg, 39% yield) as a yellow solid. LC/MS (ESI, m/z): [(M+1)]⁺=736.3.

Step 2—(S)-6-(4-(5-((5,5-difluoro-2,7-diazaspiro[3.5]nonan-2-yl)methyl)pyridin-2-yl)indolin-1-yl)-N-(2,2-difluorocyclopropyl)-8-(methylamino)imidazo[1,2-b]pyridazine-3-carboxamide trifluoroacetate. A solution of tert-butyl (S)-2-((6-(1-(3-((2,2-difluorocyclopropyl)carbamoyl)-8-(methylamino)imidazo[1,2-b]pyridazin-6-yl)indolin-4-yl)pyridin-3-yl)methyl)-5,5-difluoro-2,7-diazaspiro[3.5]nonane-7-carboxylate (60 mg, 0.082 mmol) in DCM (5 mL) and TFA (1 mL) was stirred for 1 h at rt. On completion, the mixture was concentrated under reduced pressure. The resulting mixture was diluted with diethyl ether (10 mL). The precipitated solids were collected by filtration and washed with diethyl ether (2×5 mL) to give the title compound (100 mg) as a yellow solid. LC/MS (ESI, m/z): [(M+1)]⁺=636.3.

### (R)—N-(2,2-difluorocyclopropyl)-6-(4-(5-formylpyridin-2-yl)indolin-1-yl)-8-(methylamino)imidazo[1,2-b]pyridazine-3-carboxamide trifluoroacetate (Intermediate SP)

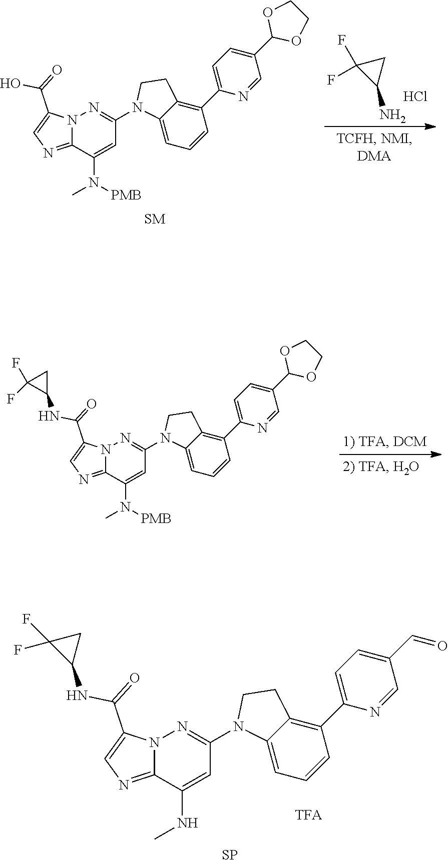

Step 1—(R)-6-(4-(5-(1,3-dioxolan-2-yl)pyridin-2-yl)indolin-1-yl)-N-(2,2-difluorocyclopropyl)-8-((4-methoxybenzyl)(methyl)amino)imidazo[1,2-b]pyridazine-3-carboxamide. To a stirred solution of 6-(4-(5-(1,3-dioxolan-2-yl)pyridin-2-yl)indolin-1-yl)-8-((4-methoxybenzyl)(methyl)amino)imidazo[1,2-b]pyridazine-3-carboxylic acid (400 mg, 0.69 mmol. Intermediate SM) and (R)-2,2-difluorocyclopropan-1-amine hydrochloride (90 mg, 0.69 mmol, CAS #2089150-96-7) in DMA (8 mL) were added TCFH (213 mg, 0.76 mmol) and 1-methyl-1H-imidazole (119 mg, 1.45 mmol) at rt. The resulting mixture was stirred for 2 h at 50° C. On completion, the mixture was cooled to rt and purified directly by reversed-phase flash chromatography (Column: Spherical C18, 20-40 μm, 330 g; Mobile Phase A: Water (plus 10 mM NH₄HCO₃), Mobile Phase B: ACN; Flow rate: 80 mL/min; Gradient (B %): 5%35%, 4 min; 35%˜80%, 20 min; 80%-95%; 2 min; 95%, 5 min; Detector: 254 nm; the fractions containing desired product were collected at 66% B and concentrated under reduced pressure) to afford the title compound (60 mg, 13% yield) as a yellow solid. LC/MS (ESI, m/z): [(M+1)]=654.5.

Step 2—(R)—N-(2,2-difluorocyclopropyl)-6-(4-(5-formylpyridin-2-yl)indolin-1-yl)-8-(methylamino)imidazo[1,2-b]pyridazine-3-carboxamide trifluoroacetate. A mixture of (R)-6-(4-(5-(1,3-dioxolan-2-yl)pyridin-2-yl)indolin-1-yl)-N-(2,2-difluorocyclopropyl)-8-((4-methoxybenzyl)(methyl)amino)imidazo[1,2-b]pyridazine-3-carboxamide (60 mg, 0.092 mmol) in TFA (2 mL) and DCM (4 mL) was stirred for 1 h at rt. The resulting mixture was concentrated under reduced pressure. To the above mixture were added H₂O (3 mL) and TFA (3 mL) at rt. The resulting mixture was stirred for additional 2 h at 50° C. On completion, the mixture was cooled to rt and neutralized to pH 8 with saturated Na₂CO₃ (aq.). The precipitated solids were collected by filtration and washed with water (3×10 mL) to afford the title compound (67 mg, TFA salt) as a yellow solid. LC/MS (ESI, m/z): [(M+1)]⁺=490.3.

### (R)-6-(4-(5-((5,5-difluoro-2,7-diazaspiro[3.5]nonan-2-yl)methyl)pyridin-2-yl)indolin-1-yl)-N-(2,2-difluorocyclopropyl)-8-(methylamino)imidazo[1,2-b]pyridazine-3-carboxamide trifluoroacetate (Intermediate SQ)

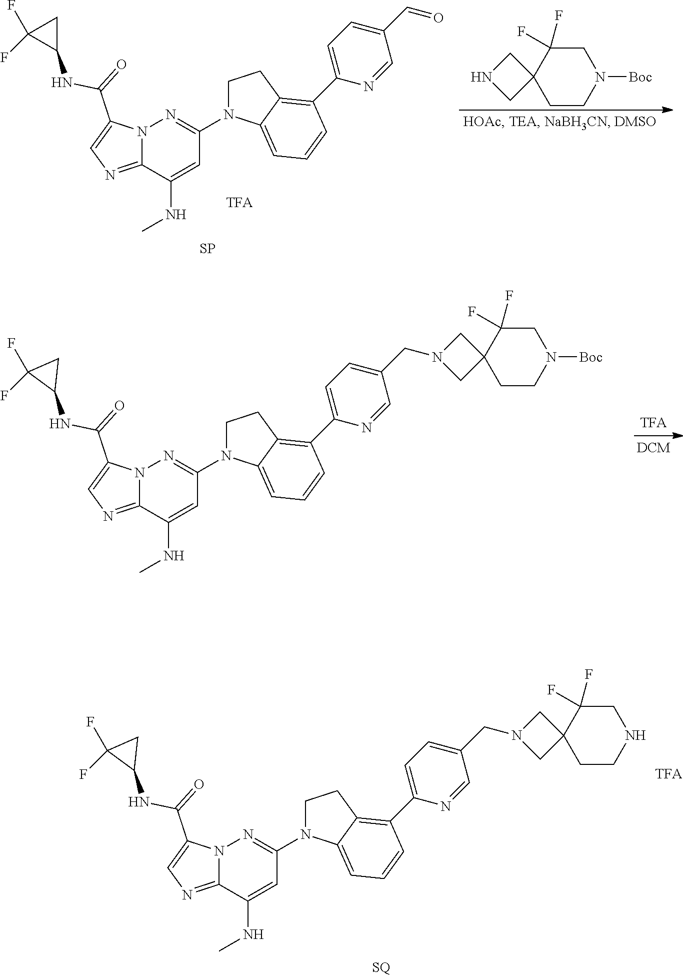

Step 1—Tert-butyl (R)-2-((6-(1-(3-((2,2-difluorocyclopropyl)carbamoyl)-8-(methylamino)imidazo[1,2-b]pyridazin-6-yl)indolin-4-yl)pyridin-3-yl)methyl)-5,5-difluoro-2,7-diazaspiro[3.5]nonane-7-carboxylate. To a stirred solution of (R)—N-(2,2-difluorocyclopropyl)-6-(4-(5-formylpyridin-2-yl)indolin-1-yl)-8-(methylamino)imidazo[1,2-b]pyridazine-3-carboxamide 2,2,2-trifluoroacetate (60 mg, 0.12 mmol, Intermediate SP) and TEA (25 mg, 0.25 mmol) in DMSO (2 mL) were added tert-butyl 5,5-difluoro-2,7-diazaspiro[3.5]nonane-7-carboxylate (32 mg, 0.12 mmol) and AcOH (73 mg, 1.23 mmol) at rt. The resulting mixture was stirred for 1 h at 50° C. To the above mixture was added NaBH₃CN (38 mg, 0.62 mmol) at rt. The resulting mixture was stirred for an additional 1 h at 50° C. On completion, the mixture was cooled to rt and purified directly by reversed-phase flash chromatography (Column: Spherical C18, 2040 μm, 120 g; Mobile Phase A: Water (plus 10 mM NH₄HCO₃), Mobile Phase B: ACN; Flow rate: 50 mL/min; Gradient (B %): 5%˜40%, 4 min; 40%˜70%, 20 min; 70%˜95%; 2 min; 95%, 5 min; Detector: 254 nm; the fractions containing desired product were collected at 66% B and concentrated under reduced pressure) to afford the title compound (34 mg, 37% yield) as a yellow solid. LC/MS (ESI, m/z): [(M+1)]⁺=736.4.

Step 2—(R)-6-(4-(5-((5,5-difluoro-2,7-diazaspiro[3.5]nonan-2-yl)methyl)pyridin-2-yl)indolin-1-yl)-N-(2,2-difluorocyclopropyl)-8-(methylamino)imidazo[1,2-b]pyridazine-3-carboxamide trifluoroacetate. A solution of tert-butyl (R)-2-((6-(1-(3-((2,2-difluorocyclopropyl)carbamoyl)-8-(methylamino)imidazo[1,2-b]pyridazin-6-yl)indolin-4-yl)pyridin-3-yl)methyl)-5,5-difluoro-2,7-diazaspiro[3.5]nonane-7-carboxylate (34 mg, 0.05 mmol) in DCM (2.5 mL) and TFA (0.5 mL) was stirred for 1 h at rt. The resulting mixture was concentrated under reduced pressure. The resulting mixture was diluted with diethyl ether (5 mL). The precipitated solids were collected by filtration and washed with diethyl ether (2×3 mL) to give the title compound (50 mg, TFA salt) as a yellow solid. LC/MS (ESI, m/z): [(M+1)]⁺=636.3.

### 1-(2-Chloro-5-(piperazine-1-carbonyl)phenyl)dihydropyrimidine-2,4(1H,3H)-dione trifluoroacetate (Intermediate SR)

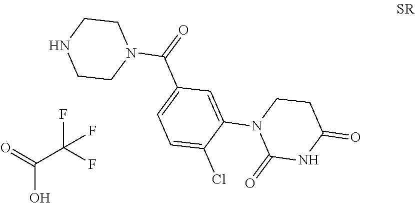

### 1-(5-(4-(azetidin-3-ylmethyl)piperazine-1-carbonyl)-2-chlorophenyl)dihydropyrimidine-2,4(1H,3H)-dione trifluoroacetate (Intermediate SS)

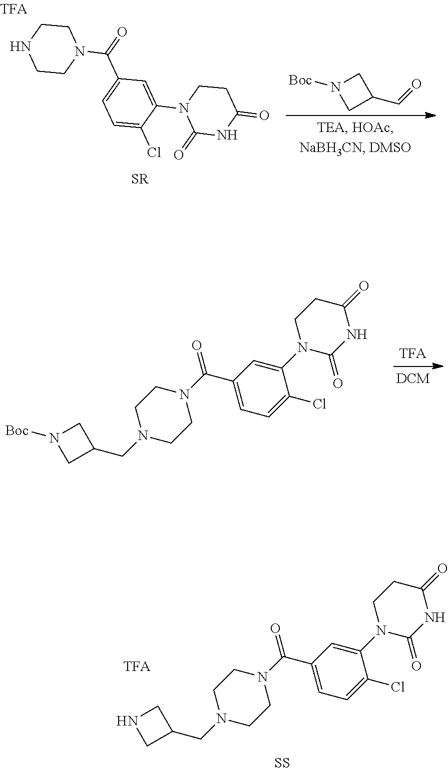

Step 1—Tert-butyl 3-((4-(4-chloro-3-(2,4-dioxotetrahydropyrimidin-1(2H)-yl)benzoyl)piperazin-1-yl)methyl)azetidine-1-carboxylate. To a stirred solution of 1-(2-chloro-5-(piperazine-1-carbonyl)phenyl)dihydropyrimidine-2,4(1H,3H)-dione trifluoroacetate (600 mg, 1.78 mmol. Intermediate SR) in DMSO (6 mL) was added TEA (541 mg, 5.35 mmol). The resulting mixture was stirred for 5 min at rt. The mixture was basified to pH 8 with TEA. To the above mixture was added tert-butyl 3-formylazetidine-1-carboxylate (330 mg, 1.78 mmol, CAS #177947-96-5) and HOAc (214 mg, 3.564 mmol) in turns at rt. The resulting mixture was stirred for additional 30 min at rt. To the above mixture was added NaBH₃CN (336 mg, 5.346 mmol) in portions over 5 min at rt. The resulting mixture was stirred for an additional 2 h at 50° C. After completion of the reaction, the reaction mixture was cooled to rt and purified directly by reverse phase flash chromatography (Column: WelFlash™ C18-I, 20-40 μm, 330 g; Eluent A: Water (plus 10 mmol/L FA); Eluent B: ACN; Gradient: 25%-55% B in 25 min; Flow rate: 80 mL/min; Detector: 220/254 nm; desired fractions were collected at 30% B and concentrated under reduced pressure) to afford the title compound (300 mg, 33% yield) as a white solid. LC/MS (ESI, m/z): [(M+H)]⁺=506.4.

Step 2—1-(5-(4-(Azetidin-3-ylmethyl)piperazine-1-carbonyl)-2-chlorophenyl)dihydropyrimidine-2,4(1H,3H)-dione trifluoroacetate. To a stirred solution of tert-butyl 3-((4-(4-chloro-3-(2,4-dioxotetrahydropyrimidin-1(2H)-yl)benzoyl)piperazin-1-yl)methyl)azetidine-1-carboxylate (300 mg, 0.593 mmol) in DCM (9 mL) was added TFA (3 mL) dropwise at rt. The resulting mixture was stirred for additional 1 h at rt. On completion, the mixture was concentrated under reduced pressure. The mixture was purified by trituration with Et₂O (10 mL) to afford the title compound (200 mg, 83% yield) as a white solid. LC/MS (ESI, m/z): [(M+H)]⁺=406.2.

### 1-(2-Chloro-5-(4-((1-(3,3-difluoropiperidin-4-yl)azetidin-3-yl)methyl)piperazine-1-carbonyl)phenyl)dihydropyrimidine-2,4(1H,3H)-dione trifluoroacetate (Intermediate ST)

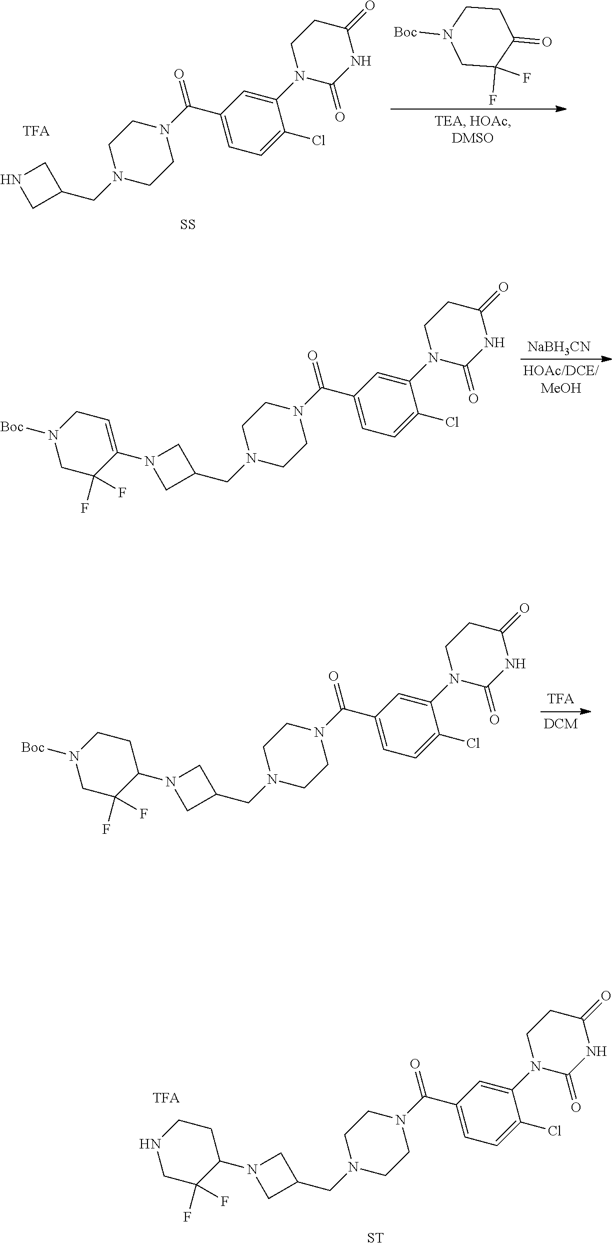

Step 1—Tert-butyl 4-(3-((4-(4-chloro-3-(2,4-dioxotetrahydropyrimidin-1(2H)-yl)benzoyl)piperazin-1-yl)methyl)azetidin-1-yl)-3,3-difluoro-3,6-dihydropyridine-1(2H)-carboxylate. To a stirred solution of 1-(5-(4-(azetidin-3-ylmethyl)piperazine-1-carbonyl)-2-chlorophenyl)dihydropyrimidine-2,4(1H,3H)-dione trifluoroacetate (300 mg, 0.595 mmol, Intermediate SS) in DMSO (10 mL) were added TEA (120 mg, 1.19 mmol) at rt. The resulting mixture was stirred for 5 min at rt. Then the mixture was basified to pH 8 to TEA. To the above mixture was added tert-butyl 3,3-difluoro-4-oxopiperidine-1-carboxylate (420.13 mg, 1.785 mmol, CAS #1215071-17-2) and HOAc (107.25 mg, 1.785 mmol) at rt. The resulting mixture was stirred for 2 h at 100° C. under nitrogen atmosphere. On completion, the mixture cooled to rt and diluted with water (200 mL). The resulting mixture was extracted with EtOAc (2×200 mL). The combined organic layers were washed with brine (2×200 mL), dried over anhydrous Na₂SO₄. After filtration, the filtrate was concentrated under reduced pressure to afford the title compound (600 mg, 81% yield) as a brown yellow oil. LC/MS (ESI, m/z): [(M+H)]⁺=623.2.

Step 2—Tert-butyl 4-(3-((4-(4-chloro-3-(2,4-dioxotetrahydropyrimidin-1(2H)-yl)benzoyl)piperazin-1-yl)methyl)azetidin-1-yl)-3,3-difluoropiperidine-1-carboxylate. To a stirred solution of tert-butyl 4-(3-((4-(4-chloro-3-(2,4-dioxotetrahydropyrimidin-1(2H)-yl)benzoyl)piperazin-1-yl)methyl)azetidin-1-yl)-3,3-difluoro-3,6-dihydropyridine-1(2H)-carboxylate (600 mg, 0.963 mmol) in DCE (10 mL) and MeOH (10 mL) were added HOAc (1 mL) and NaBH₃CN (303 mg, 4.815 mmol) in portions at rt. The resulting mixture was stirred overnight at rt under nitrogen atmosphere. On completion, the mixture was concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 35%-55% B in 20 min; Flow rate: 80 mL/min; Detector: 220/254 nm; desired fractions were collected at 46% B and concentrated under reduced pressure) to afford the title compound (20 mg, 3% yield) as a white solid. LC/MS (ESI, m/z): [(M+H)]⁺=625.4.

Step 3—1-(2-Chloro-5-(4-((1-(3,3-difluoropiperidin-4-yl)azetidin-3-yl)methyl)piperazine-1-carbonyl)phenyl)dihydropyrimidine-2,4(1H,3H)-dione trifluoroacetate. To a solution of tert-butyl 4-(3-((4-(4-chloro-3-(2,4-dioxotetrahydropyrimidin-1(2H)-yl)benzoyl)piperazin-1-yl)methyl)azetidin-1-yl)-3,3-difluoropiperidine-1-carboxylate (20 mg, 0.032 mmol) in DCM (0.9 mL) was added TFA (0.3 mL) dropwise rt and the mixture was stirred for 1 h at rt under nitrogen atmosphere. The resulting mixture was concentrated under reduced pressure. The residue was purified by trituration with Et₂O (20 mL) to give the title compound (20 mg, 90% yield) as a light yellow solid. LC/MS (ESI, m/z): [(M+H)]⁺=525.4.

### 6-(4-(5-formylpyrazin-2-yl)indolin-1-yl)-N-((1R,2R)-2-methoxycyclobutyl)-8-(methylamino)imidazo[1,2-b]pyridazine-3-carboxamide (Intermediate SU)

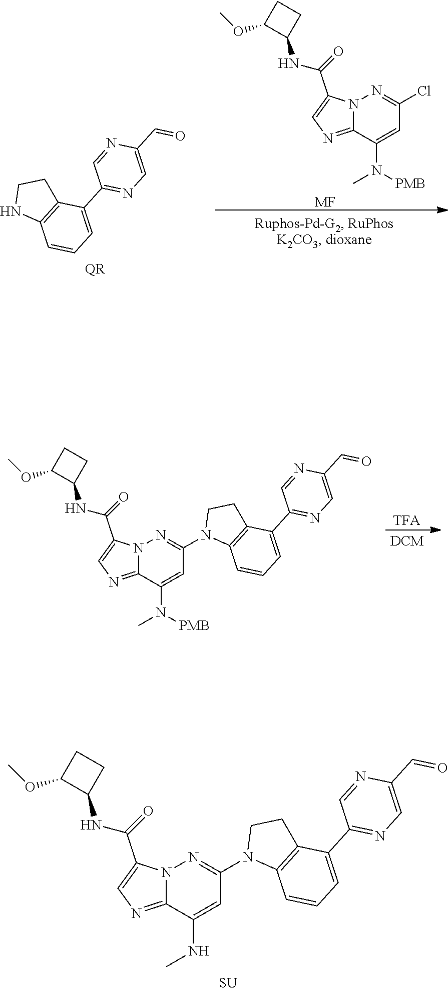

Step 1—6-(4-(5-Formylpyrazin-2-yl)indolin-1-yl)-8-((4-methoxybenzyl)(methyl)amino)-N-((1R,2R)-2-methoxycyclobutyl)imidazo[1,2-b]pyridazine-3-carboxamide. To a stirred solution of 5-(2,3-dihydro-1H-indol-4-yl)pyrazine-2-carbaldehyde (300 mg, 1.33 mmol, Intermediate QR) and 6-chloro-N-[(1R,2R)-2-methoxycyclobutyl]-8-{[(4-methoxyphenyl)methyl](methyl)amino}imidazo[1,2-b]pyridazine-3-carboxamide (572 mg, 1.33 mmol, Intermediate MF), RuPhos (124 mg, 0.266 mmol) and RuPhos-PdCl-2nd G (124 mg, 0.266 mmol) in dioxane (5 mL) was added K₂CO₃ (552 mg, 4.00 mmol) dropwise at rt under nitrogen atmosphere. The resulting mixture was stirred for 2 h at 80° C. under nitrogen atmosphere. On completion, the mixture was cooled to rt and concentrated under reduced pressure. The mixture was purified by silica gel column chromatography (Mobile Phase A: PE, Mobile Phase B: EA; Gradient: 0% to 50% B in 25 min, 254 nm; the fractions containing the desired product were collected at 20% B and concentrated under reduced pressure) to afford the title compound (270 mg, 33% yield) as an orange solid. LC/MS (ESI, m/z): [(M+H)]⁺=619.3.

Step 2—6-(4-(5-Formylpyrazin-2-yl)indolin-1-yl)-N-((1R,2R)-2-methoxycyclobutyl)-8-(methylamino)imidazo[1,2-b]pyridazine-3-carboxamide. To a stirred solution of 6-[4-(5-formylpyrazin-2-yl)-2,3-dihydroindol-1-yl]-N-[(1R,2R)-2-methoxycyclobutyl]-8-{[(4-methoxyphenyl)methyl](methyl)amino}imidazo[1,2-b]pyridazine-3-carboxamide (270 mg, 0.430 mmol) in DCM (6 mL) was added TFA (2 mL, 27 mmol) dropwise at rt under air atmosphere. The resulting mixture was stirred for 1 h at rt under air atmosphere. On completion, the mixture was concentrated under reduced pressure. The mixture was purified by trituration with Et₂O (10 mL) to afford to afford the title compound (210 mg, 96% yield) as an orange solid. LC/MS (ESI, m/z): [(M+H)]⁺=499.2.

### 6-(4-(5-((1,1-Difluoro-3,9-diazaspiro[5.5]undecan-3-yl)methyl)pyrazin-2-yl)indolin-1-yl)-N-((1R,2R)-2-methoxycyclobutyl)-8-(methylamino)imidazo[1,2-b]pyridazine-3-carboxamide trifluoroacetate (Intermediate SV)

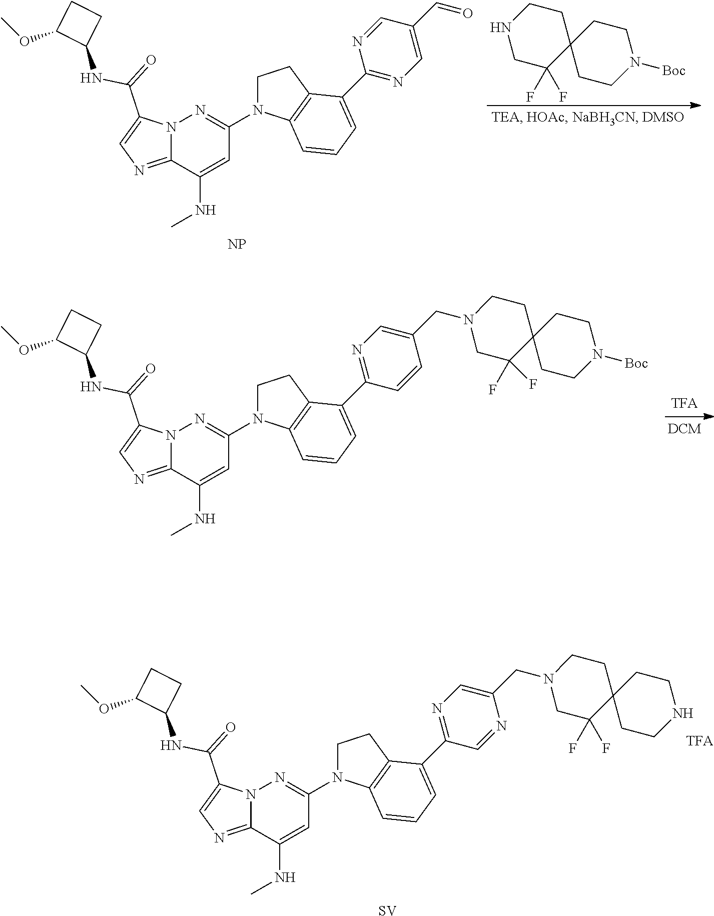

Step 1—Tert-butyl 7,7-difluoro-9-((5-(1-(3-(((1R,2R)-2-methoxycyclobutyl)carbamoyl)-8-(methylamino)imidazo[1,2-b]pyridazin-6-yl)indolin-4-yl)pyrazin-2-yl)methyl)-3,9-diazaspiro[5.5]undecane-3-carboxylate. To a stirred solution of tert-butyl 7,7-difluoro-3,9-diazaspiro[5.5]undecane-3-carboxylate (76 mg, 0.26 mmol, CAS #1784848-04-9) in DMSO (5 mL) were added TEA (53 mg, 0.522 mmol) at rt under nitrogen atmosphere and the mixture was stirred for 5 min at rt. The mixture was basified to pH 8 with TEA. To the above mixture was added 6-(4-(5-formylpyrazin-2-yl)indolin-1-yl)-N-((1R,2R)-2-methoxycyclobutyl)-8-(methylamino)imidazo[1,2-b]pyridazine-3-carboxamide (130 mg, 0.261 mmol, Intermediate SU) and HOAc (47 mg, 0.783 mmol) at rt. The resulting mixture was stirred for 30 min at rt. To the above mixture was added NaBH₃CN (49 mg, 0.783 mmol) in portions over 5 min at rt. The resulting mixture was stirred for 2 h at 50° C. under nitrogen atmosphere. After completion of the reaction, the reaction mixture was cooled to rt and purified directly by reverse phase flash chromatography (Column: WelFlash™ C18-I, 20-40 μm, 120 g; Eluent A: Water (plus 10 mmol/L NH₄HCO₃); Eluent B: ACN; Gradient: 40%-60% B in 20 min; Flow rate: 60 mL/min; Detector: 220/254 nm; desired fractions were collected at 53% B and concentrated under reduced pressure) to afford the title compound (70 mg, 35% yield) as a light yellow solid. LC/MS (ESI, m/z): [(M+H)]⁺=773.4.

Step 2—6-(4-(5-((1,1-Difluoro-3,9-diazaspiro[5.5]undecan-3-yl)methyl)pyrazin-2-yl)indolin-1-yl)-N-((1R,2R)-2-methoxycyclobutyl)-8-(methylamino)imidazo[1,2-b]pyridazine-3-carboxamide trifluoroacetate. To a solution of tert-butyl 7,7-difluoro-9-((5-(1-(3-(((1R,2R)-2-methoxycyclobutyl)carbamoyl)-8-(methylamino)imidazo[1,2-b]pyridazin-6-yl)indolin-4-yl)pyrazin-2-yl)methyl)-3,9-diazaspiro[5.5]undecane-3-carboxylate (70 mg, 0.091 mmol) in DCM (9 mL) was added TFA (3 mL) dropwise at rt and the mixture was stirred for 1 h at rt under nitrogen atmosphere. On completion, the mixture was concentrated under reduced pressure. The residue was purified by trituration with Et₂O (30 mL) to give the title compound (60 mg, 84% yield) as a brown yellow solid. LC/MS (ESI, m/z): [(M+H)]⁺=673.3.

### Tert-butyl 9-((6-bromopyridazin-3-yl)methyl)-7,7-difluoro-3,9-diazaspiro[5.5]undecane-3-carboxylate (Intermediate SW)

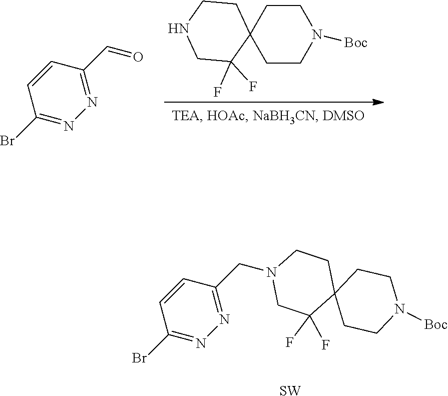 [SMILES: O=Cc1ccc(Br)nn1 ; CC(C)(C)OC(=O)N1CCC2(CCNCC2(F)F)CC1 ; CC(C)(C)OC(=O)N1CCC2(CCN(Cc3ccc(Br)nn3)CC2(F)F)CC1 ; "SW"]

To a stirred solution of tert-butyl 7,7-difluoro-3,9-diazaspiro[5.5]undecane-3-carboxylate (512 mg, 1.76 mmol, CAS #1784848-04-9) in DMSO (6 mL) were added TEA (325 mg, 3.21 mmol) at rt under nitrogen atmosphere and the mixture was stirred for 5 min at rt. The mixture was basified to pH 8. To the above mixture was added 6-bromopyridazine-3-carbaldehyde (300 mg, 1.60 mmol, CAS #2169110-11-4) and HOAc (289 mg, 4.812 mmol) at rt and the mixture was stirred for an additional 30 min at rt. To the above mixture was added NaBH₃CN (302 mg, 4.812 mmol) in portions over 5 min at rt. The resulting mixture was stirred for 2 h at 50° C. under nitrogen atmosphere. After completion of the reaction, the reaction mixture was cooled to rt and purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 35%-55% B in 25 min; Flow rate: 80 mL/min; Detector: 220/254 nm; desired fractions were collected at 45% B and concentrated under reduced pressure) to afford the title compound (370 mg, 50%) as a brown solid. LC/MS (ESI, m/z): [(M+H)]⁺=461.1, 463.1.

### 6-(4-(6-((1,1-Difluoro-3,9-diazaspiro[5.5]undecan-3-yl)methyl)pyridazin-3-yl)indolin-1-yl)-N-((1R,2S)-2-fluorocyclopropyl)-8-(methylamino)imidazo[1,2-b]pyridazine-3-carboxamide trifluoroacetate (Intermediate SX)

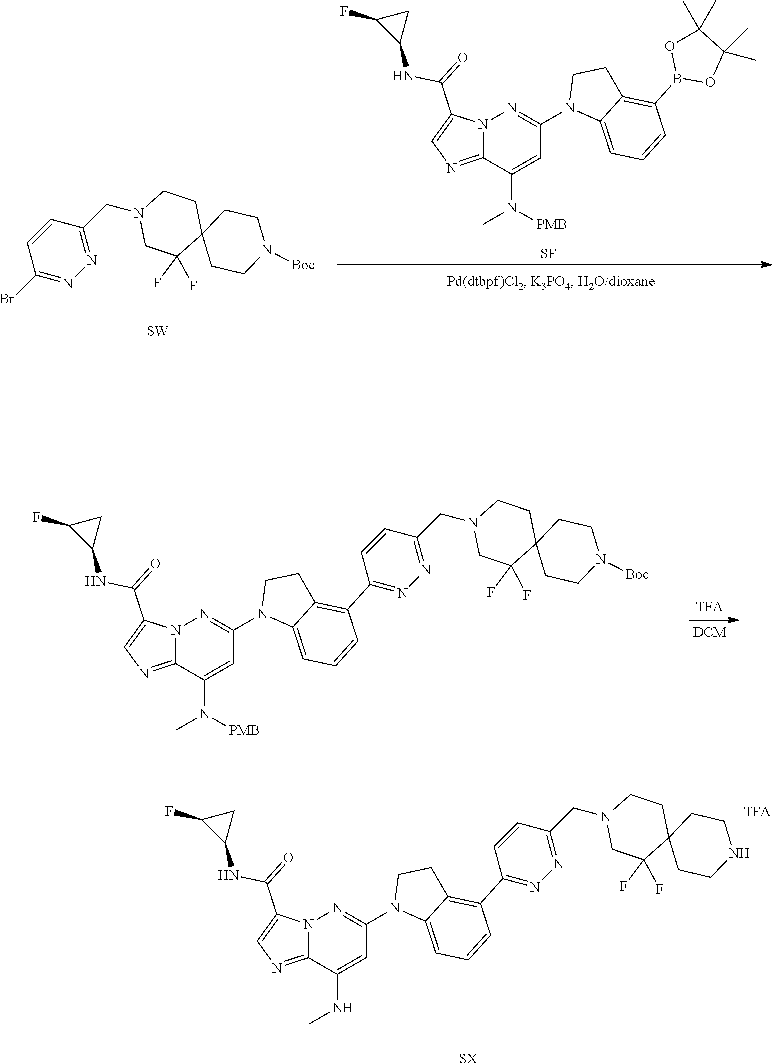

Step 1—Tert-butyl 7,7-difluoro-9-((6-(1-(3-(((1R,2S)-2-fluorocyclopropyl)carbamoyl)-8-((4-methoxybenzyl)(methyl)amino)imidazo[1,2-b]pyridazin-6-yl)indolin-4-yl)pyridazin-3-yl)methyl)-3,9-diazaspiro[5.5]undecane-3-carboxylate. To a stirred solution of tert-butyl 9-((6-bromopyridazin-3-yl)methyl)-7,7-difluoro-3,9-diazaspiro[5.5]undecane-3-carboxylate (150 mg, 0.325 mmol, Intermediate SW) and N-((1R,2S)-2-fluorocyclopropyl)-8-((4-methoxybenzyl)(methyl)amino)-6-(4-(4,4,5,5-tetramethyl-1,3,2-dioxaborolan-2-yl)indolin-1-yl)imidazo[1,2-b]pyridazine-3-carboxamide (597 mg, 0.975 mmol, Intermediate SF) in dioxane (10 mL) and H₂O (2 mL) were added K₃PO₄ (207 mg, 0.975 mmol) and Pd(DtBPF)Cl₂ (42 mg, 0.065 mmol) in turns at rt under nitrogen atmosphere. The resulting mixture was stirred for 2 h at 90° C. under nitrogen atmosphere. On completion, the mixture was cooled to rt and concentrated under reduced pressure. The crude product was purified by silica gel column chromatography (Mobile Phase A: Petroleum ether, Mobile Phase B: EtOAc; Gradient: 70% B to 100% B in 20 min, 254 nm; the fractions containing the desired product were collected at 100% B) to afford the title compound (100 mg, 35% yield) as a brown oil. LC/MS (ESI, m/z): [(M+H)]⁺=867.4.

Step 2—6-(4-(6-((1,1-Difluoro-3,9-diazaspiro[5.5]undecan-3-yl)methyl)pyridazin-3-yl)indolin-1-yl)-N-((1R,2S)-2-fluorocyclopropyl)-8-(methylamino)imidazo[1,2-b]pyridazine-3-carboxamide trifluoroacetate. To a solution of tert-butyl 7,7-difluoro-9-((6-(1-(3-(((1R,2S)-2-fluorocyclopropyl)carbamoyl)-8-((4-methoxybenzyl)(methyl)amino)imidazo[1,2-b]pyridazin-6-yl)indolin-4-yl)pyridazin-3-yl)methyl)-3,9-diazaspiro[5.5]undecane-3-carboxylate (100 mg, 0.115 mmol) in DCM (12 mL) was added TFA (4 mL) dropwise at rt. The reaction mixture was stirred for 1 h at rt under nitrogen atmosphere. On completion, the mixture was concentrated under reduced pressure. The residue was purified by trituration with Et₂O (30 mL) to afford the title compound (170 mg, 98% yield) as a brown solid. LC/MS (ESI, m/z): [(M+H)]⁺=647.3.

### 6-(4-(5-((6,6-Difluoro-2,8-diazaspiro[4.5]decan-8-yl)methyl)pyridin-2-yl)indolin-1-yl)-N-((1R,2S)-2-fluorocyclopropyl)-8-(methylamino)imidazo[1,2-b]pyridazine-3-carboxamide (Intermediate SY)

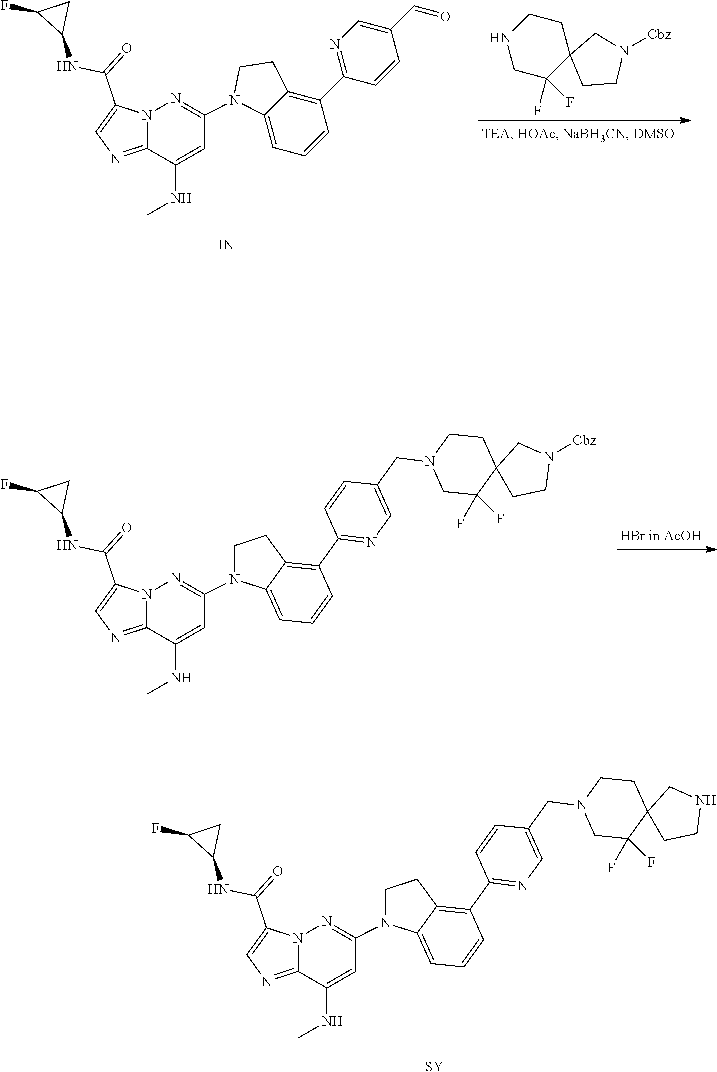 [SMILES: CNc1cc(N2CCc3c(-c4ccc(C=O)cn4)cccc32)nn2c(C(=O)N[C@@H]3C[C@@H]3F)cnc12 ; O=C(OCc1ccccc1)N1CCC2(CCNCC2(F)F)C1 ; "IN" ; CNc1cc(N2CCc3c(-c4ccc(CN5CCC6(CCN(C(=O)OCc7ccccc7)C6)C(F)(F)C5)cn4)cccc32)nn2c(C(=O)N[C@@H]3C[C@@H]3F)cnc12 ; CNc1cc(N2CCc3c(-c4ccc(CN5CCC6(CCNC6)C(F)(F)C5)cn4)cccc32)nn2c(C(=O)N[C@@H]3C[C@@H]3F)cnc12 ; "SY"]

Step 1—Benzyl 6,6-difluoro-8-((6-(1-(3-(((1R,2S)-2-fluorocyclopropyl)carbamoyl)-8-(methylamino)imidazo[1,2-b]pyridazin-6-yl)indolin-4-yl)pyridin-3-yl)methyl)-2,8-diazaspiro[4.5]decane-2-carboxylate. To a stirred solution of benzyl 6,6-difluoro-2,8-diazaspiro[4.5]decane-2-carboxylate (300 mg, 0.967 mmol, CAS #1894944-16-1) in DMSO (10 mL) were added TEA (196 mg, 1.934 mmol) at rt under nitrogen atmosphere and the mixture was stirred for 5 min at rt. The mixture was basified to pH 8. To the above mixture was added N-((1R,2S)-2-fluorocyclopropyl)-6-(4-(5-formylpyridin-2-yl)indolin-1-yl)-8-(methylamino)imidazo[1,2-b]pyridazine-3-carboxamide (456 mg, 0.967 mmol, Intermediate IN) and HOAc (174 mg, 2.90 mmol) at rt. The resulting mixture was stirred for an additional 30 min at rt. To the above mixture was added NaBH₃CN (182 mg, 2.901 mmol) in portions over 5 min at rt. The resulting mixture was stirred for 2 h at 50° C. under nitrogen atmosphere. After completion of the reaction, the reaction mixture was cooled to it and purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 50%-70% B in 25 min; Flow rate: 80 mL/min; Detector: 220/254 nm; desired fractions were collected at 66% B and concentrated under reduced pressure) to afford the title compound (150 mg, 20% yield) as a white solid. LC/MS (ESI, m/z): [(M+H)]f=766.4.

Step 2—6-(4-(5-((6,6-Difluoro-2,8-diazaspiro[4.5]decan-8-yl)methyl)pyridin-2-yl)indolin-1-yl)-N-((1R,2S)-2-fluorocyclopropyl)-8-(methylamino)imidazo[1,2-b]pyridazine-3-carboxamide. A solution of benzyl 6,6-difluoro-8-((6-(1-(3-(((1R,2S)-2-fluorocyclopropyl)carbamoyl)-8-(methylamino)imidazo[1,2-b]pyridazin-6-yl)indolin-4-yl)pyridin-3-yl)methyl)-2,8-diazaspiro[4.5]decane-2-carboxylate (150 mg, 0.196 mmol) in HBr in CH₃COOH (40%) (10 mL) was stirred for 1 h at rt. On completion, the mixture was concentrated under reduced pressure. The residue was purified by reverse flash chromatography (Column: WelFlash™ C18-I, 20-40 μm, 120 g; Eluent A: Water (plus 10 mmol/L FA); Eluent B: ACN; Gradient: 5%-25% B in 25 min; Flow rate: 50 mL/min; Detector: 220/254 nm; desired fractions were collected at 10% B and concentrated under reduced pressure) to afford the title compound (80 mg, 65% yield) as a brown oil. LC/MS (ESI, m/z): [(M+H)]⁺=632.3.

### 4-(2,4-Dioxotetrahydropyrimidin-1(2H)-yl)-5-methylpicolinic acid (Intermediate SZ)

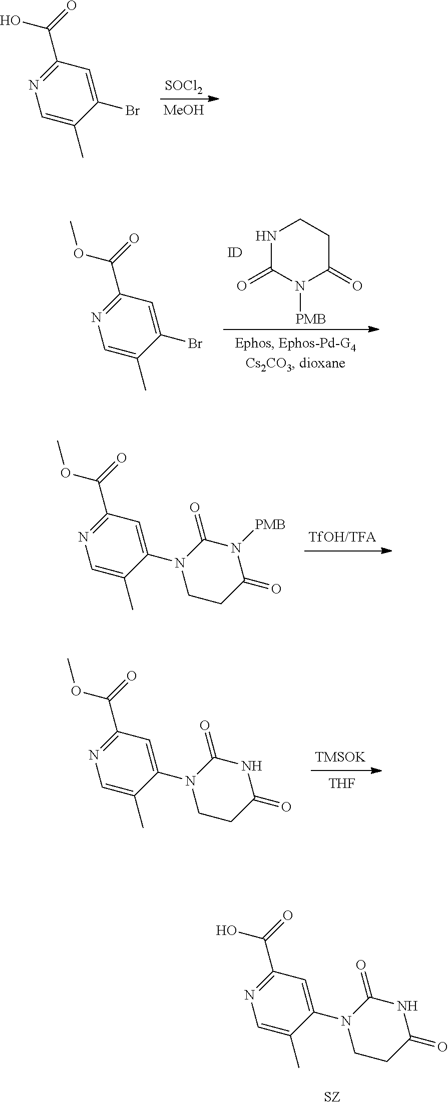

Step 1—Methyl 4-bromo-5-methylpicolinate. To a stirred solution of 4-bromo-5-methylpicolinic acid (1.00 g, 4.63 mmol, CAS #1196154-93-4) in MeOH (15 mL) was added sulfurous dichloride (4.41 g, 37.0 mmol) dropwise at rt under air atmosphere. The resulting mixture was stirred for 4 h at 80° C. under N₂ atmosphere. On completion, the mixture was cooled to rt and concentrated under reduced pressure. The mixture was extracted with EtOAc (3×100 mL) and H₂O (300 mL). The combined organic layers were washed with brine (2×100 mL), dried over anhydrous Na₂SO₄. After filtration, the filtrate was concentrated under reduced pressure to give the title compound (827 mg, 78% yield) as a yellow solid. LC/MS (ESI, m/z): [(M+H)]f=230.0, 232.0

Step 2—Methyl 4-(3-(4-methoxybenzyl)-2,4-dioxotetrahydropyrimidin-1(2H)-yl)-5-methylpicolinate. To a stirred solution of methyl 4-bromo-5-methylpicolinate (600 mg, 2.56 mmol), 3-(4-methoxybenzyl)dihydropyrimidine-2,4(1H,3H)-dione (589 mg, 2.561 mmol, Intermediate ID) and Cs₂CO₃ (2.5 g, 7.68 mmol) in dioxane (10 mL) were added EPhos (274 mg, 0.512 mmol) and EPhos Pd G4 (471 mg, 0.512 mmol) in turns at rt under nitrogen atmosphere. The resulting mixture was stirred for 4 h at 80° C. under nitrogen atmosphere. On completion, the mixture was cooled to rt and concentrated under reduced pressure. The crude product was purified by silica gel column chromatography (Mobile Phase A: Petroleum ether, Mobile Phase B: EtOAc; Gradient: 50% B to 100% B in 25 min, 254 nm; the fractions containing the desired product were collected at 70% B) to afford the title compound (100 mg, 10% yield) as a white solid. LC/MS (ESI, m/z): [(M+H)]⁺=384.2.

Step 3—Methyl 4-(2,4-dioxotetrahydropyrimidin-1(2H)-yl)-5-methylpicolinate. To a stirred solution of methyl 4-(3-(4-methoxybenzyl)-2,4-dioxotetrahydropyrimidin-1(2H)-yl)-5-methylpicolinate (100 mg, 0.26 mmol) in TFA (4 mL) was added trifluoromethanesulfonic acid (0.2 mL) dropwise at rt under air atmosphere and the mixture was stirred for 1 h at 80° C. under air atmosphere. On completion, the mixture was cooled to rt and was basified to pH 8 with saturated ammonium bicarbonate aqueous solution. The resulting mixture was concentrated under reduced pressure. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 10%-30% B in 20 min; Flow rate: 60 mL/min; Detector: 220/254 nm; desired fractions were collected at 21% B and concentrated under reduced pressure) to afford the title compound (39 mg, 57% yield) as a white solid. LC/MS (ESI, m/z): [(M+H)]f=264.2.

Step 4—4-(2,4-Dioxotetrahydropyrimidin-1(2H)-yl)-5-methylpicolinic acid. To a stirred solution of methyl 4-(2,4-dioxotetrahydropyrimidin-1(2H)-yl)-5-methylpicolinate (35 mg, 0.13 mmol) in THF (3 mL) was added potassium trimethylsilanolate (85 mg, 0.67 mmol) at rt and the mixture was stirred for 1 h at rt. On completion, the mixture was acidified to pH 6 with FA. The resulting mixture was concentrated under reduced pressure. The residue was purified by reverse phase flash chromatography (Column: WelFlash™ C18-I, 20-40 μm, 40 g; Eluent A: Water (plus 10 mmol/L NH₄HCO₃); Eluent B: ACN; Gradient: 5%-20% B in 20 min; Flow rate: 30 mL/min; Detector: 220/254 nm; desired fractions were collected at 10% B and concentrated under reduced pressure) to afford the title compound (18 mg, 54% yield) as a white solid. LC/MS (ESI, m/z): [(M+H)]⁺=250.2.

### 1-(2-Chloro-5-(methyl(3-azaspiro[5.5]undecan-9-yl)amino)phenyl)dihydropyrimidine-2,4(1H,3H)-dione (Intermediate TA)

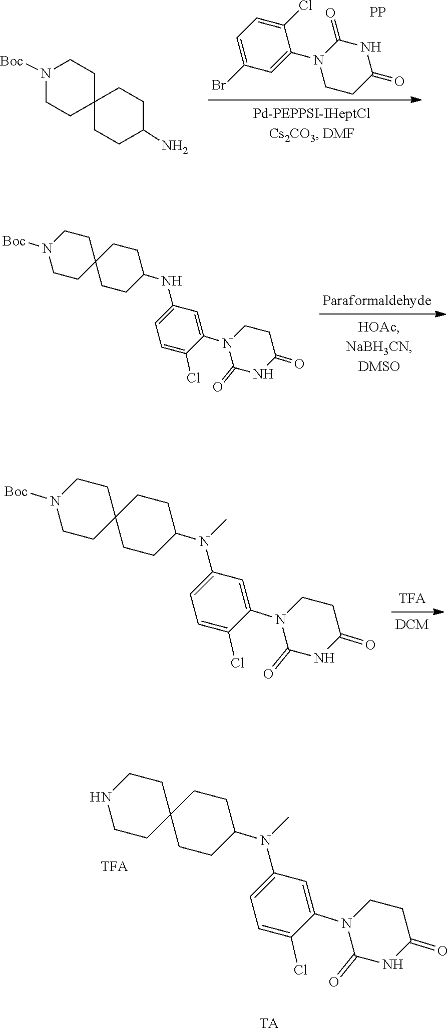

Step 1—Tert-butyl 9-((4-chloro-3-(2,4-dioxotetrahydropyrimidin-1(2H)-yl)phenyl)amino)-3-azaspiro[5.5]undecane-3-carboxylate. To a stirred solution of tert-butyl 9-amino-3-azaspiro[5.5]undecane-3-carboxylate (884 mg, 3.29 mmol, CAS #1272758-41-4) and 1-(5-bromo-2-chlorophenyl)dihydropyrimidine-2,4(1H,3H)-dione (9994 mg, 3.29 mmol, Intermediate PP) in DMF (20 mL) were added Cs₂CO₃ (3.22 g, 9.88 mmol) and (1,3-bis[2,6-bis(pentan-3-yl)phenyl]-4,5-dichloro-2,3-dihydro-1H-imidazol-2-yldichloro(2-methyl-1lambda4-pyridin-1-yl)palladium (277 mg, 0.33 mmol) at rt under nitrogen atmosphere. The resulting mixture was stirred for 16 h at 100° C. under nitrogen atmosphere. On completion, the mixture was cooled to it and purified directly by reversed-phase flash chromatography (Column: Spherical C18, 20-40 μm, 330 g; Mobile Phase A: water (plus 10 mM NH₄HCO₃), Mobile Phase B: ACN; Flow rate: 80 mL/min; Gradient (B %): 5%-40%, 4 min; 40%-80%, 20 min; 80%-95%; 2 min; 95%, 5 min; Detector: 254 nm; the fractions containing desired product were collected at 72% B and concentrated under reduced pressure) to afford the title compound (350 mg, 21% yield) as a brown solid. LC/MS (ESI, m/z): [(M+1)]⁺=491.3.

Step 2—Tert-butyl 9-((4-chloro-3-(2,4-dioxotetrahydropyrimidin-1(2H)-yl)phenyl)(methyl)amino)-3-azaspiro[5.5]undecane-3-carboxylate. To a stirred solution of tert-butyl 9-((4-chloro-3-(2,4-dioxotetrahydropyrimidin-1(2H)-yl)phenyl)amino)-3-azaspiro[5.5]undecane-3-carboxylate (350 mg, 0.71 mmol) and paraformaldehyde (96 mg, 1.07 mmol) in DMSO (4 mL) was added AcOH (128 mg, 2.14 mmol) at rt and the mixture was stirred for 1 h at rt. To the above mixture was added NaBH₃CN (179 mg, 2.85 mmol) at rt and the mixture was stirred for an additional 16 h at rt. On completion, the mixture was purified by reversed-phase flash chromatography (Column: Spherical C18, 20-40 μm, 120 g; Mobile Phase A: water (plus 10 mM NH₄HCO₃). Mobile Phase B: ACN; Flow rate: 50 mL/min; Gradient (B %): 5%-22%, 4 min; 22%-40%, 20 min; 40%-95%; 2 min; 95%, 5 min; Detector: 254 nm; the fractions containing desired product were collected at 56% B and concentrated under reduced pressure) to afford the title compound (330 mg, 91% yield) as a white solid. LC/MS (ESI, m/z): [(M+1)]=505.4.

Step 3—1-(2-Chloro-5-(methyl(3-azaspiro[5.5]undecan-9-yl)amino)phenyl)dihydropyrimidine-2,4(1H,3H)-dione. A solution of tert-butyl 9-((4-chloro-3-(2,4-dioxotetrahydropyrimidin-1(2H)-yl)phenyl)(methyl)amino)-3-azaspiro[5.5]undecane-3-carboxylate (330 mg, 0.65 mmol) in DCM (5 mL) and TFA (1 mL) was stirred for 1 h at rt. On completion, the mixture was concentrated under reduced pressure. The mixture was basified to pH 7 with saturated NaHCO₃ (aq) and the mixture was extracted with EtOAc (3×50 mL). The combined organic layers were washed with brine (1×100 mL), dried over anhydrous Na₂SO₄. After filtration, the filtrate was concentrated under reduced pressure to give the title compound (300 mg) as an off-white solid. LC/MS (ESI, m/z): [(M+1)]⁺=405.3.

### 1-(2-Chloro-5-((3-(3,3-difluoropiperidin-4-yl)-3-azaspiro[5.5]undecan-9-yl)(methyl)amino)phenyl)dihydropyrimidine-2,4(1H,3H)-dione trifluoroacetate (Intermediate TB)

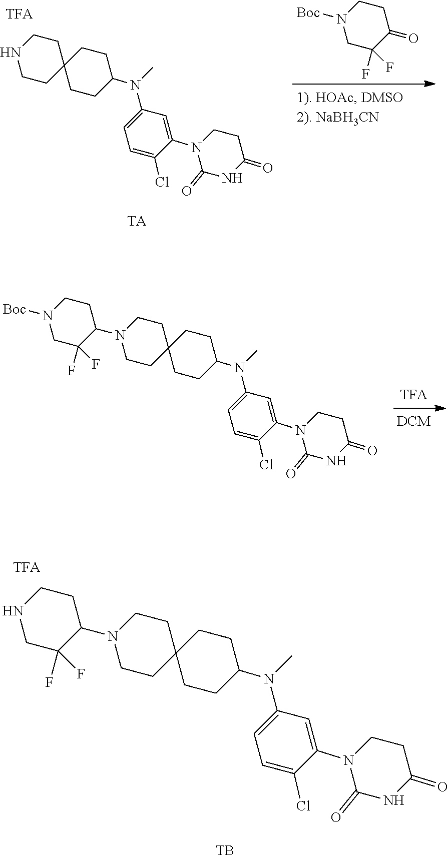 [SMILES: CN(c1ccc(Cl)c(N2CCC(=O)NC2=O)c1)C1CCC2(CCNCC2)CC1 ; CC(C)(C)OC(=O)N1CCC(=O)C(F)(F)C1 ; "TA" ; CN(c1ccc(Cl)c(N2CCC(=O)NC2=O)c1)C1CCC2(CC1)CCN(C1CCN(C(=O)OC(C)(C)C)CC1(F)F)CC2 ; CN(c1ccc(Cl)c(N2CCC(=O)NC2=O)c1)C1CCC2(CC1)CCN(C1CCNCC1(F)F)CC2 ; "TB"]

Step 1—Tert-butyl 4-(9-((4-chloro-3-(2,4-dioxotetrahydropyrimidin-1(2H)-yl)phenyl)(methyl)amino)-3-azaspiro[5.5]undecan-3-yl)-3,3-difluoropiperidine-1-carboxylate. A solution of 1-(2-chloro-5-(methyl(3-azaspiro[5.5]undecan-9-yl)amino)phenyl)dihydropyrimidine-2,4(1H,3H)-dione 2,2,2-trifluoroacetate (300 mg, 0.74 mmol. Intermediate TA), AcOH (222 mg, 3.71 mmol) and tert-butyl 3,3-difluoro-4-oxopiperidine-1-carboxylate (174 mg, 0.74 mmol) in DMSO (6 mL) was stirred for 1 h at 100° C. To the above mixture was added NaBH₃CN (232 mg, 3.71 mmol) at rt. The resulting mixture was stirred for additional 16 h at 50° C. On completion, the mixture was purified directly by reversed-phase flash chromatography (Column: Spherical C18, 20-40 μm, 330 g; Mobile Phase A: Water (plus 10 mM NH₄HCO₃), Mobile Phase B: ACN; Flow rate: 80 mL/min; Gradient (B %): 5%-40%, 4 min; 40%-80%, 20 min; 80%-95%; 2 min; 95%, 5 min; Detector: 254 nm; the fractions containing desired product were collected at 65% B and concentrated under reduced pressure) to afford the title compound (200 mg, 43% yield) as a white solid. LC/MS (ESI, m/z): [(M+1)]⁺=624.4.

Step 2—1-(2-Chloro-5-((3-(3,3-difluoropiperidin-4-yl)-3-azaspiro[5.5]undecan-9-yl)(methyl)amino)phenyl)dihydropyrimidine-2,4(1H,3H)-dione trifluoroacetate. A solution of tert-butyl 4-(9-((4-chloro-3-(2,4-dioxotetrahydropyrimidin-1(2H)-yl)phenyl)(methyl)amino)-3-azaspiro[5.5]undecan-3-yl)-3,3-difluoropiperidine-1-carboxylate (200 mg, 0.32 mmol) in DCM (10 mL) and TFA (2 mL) was stirred for 1 h at rt. On completion, the mixture was concentrated under reduced pressure. The resulting mixture was diluted with diethyl ether (10 mL). The precipitated solids were collected by filtration and washed with diethyl ether (2×5 mL) to give the title compound (300 mg, TFA salt) as a white solid. LC/MS (ESI, m/z): [(M+1)]⁺=524.4.

### 2-bromo-6-[(3R)-3-methoxyoxolan-3-yl]-4-methylpyridine (Intermediate TC) and 2-bromo-6-[(3S)-3-methoxyoxolan-3-yl]-4-methylpyridine (Intermediate TD)

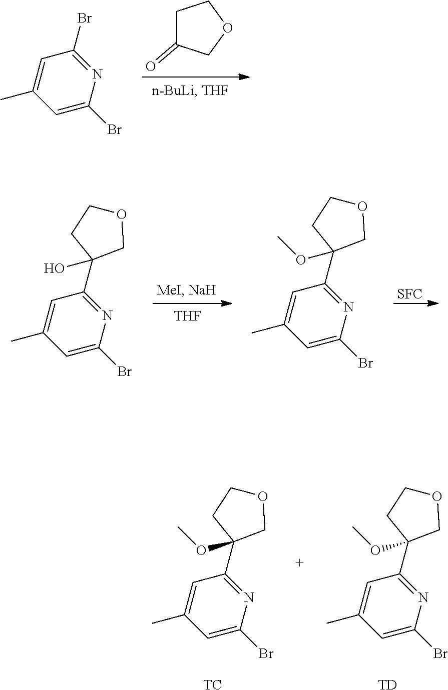

Step 1—3-(6-Bromo-4-methylpyridin-2-yl)oxolan-3-ol. To a stirred solution of 2,6-dibromo-4-methylpyridine (2 g, 8 mmol) in DCM (40 mL) was added n-BuLi (3.52 mL, 8.80 mmol) dropwise at −78° C. under nitrogen atmosphere. The resulting mixture was stirred for 30 min at −78° C. under nitrogen atmosphere. To the above mixture was added dihydrofuran-3-one (0.82 g, 9.525 mmol) at −78° C. The resulting mixture was stirred for an additional 2 h at −78° C. On completion, the reaction was quenched by the addition of sat. NH₄Cl (aq.) (50 mL) at rt. The resulting mixture was extracted with EtOAc (3×100 mL). The combined organic layers were washed with brine (1×100 mL), and dried over anhydrous Na₂SO₄. After filtration, the filtrate was concentrated under reduced pressure. The residue was purified by silica gel column chromatography, eluted with PE/EA (37%), to afford the title compound (1.79 g, 87% yield) as a white solid. LC/MS (ESI, m/z): [(M+H)]⁺=258.0, 260.0.

Step 2—2-Bromo-6-(3-methoxyoxolan-3-yl)-4-methylpyridine. To a stirred solution of 3-(6-bromo-4-methylpyridin-2-yl)oxolan-3-ol (1.5 g, 5.8 mmol) in THF (50 mL) was added NaH (0.35 g, 8.7 mmol, 60% dispersion in mineral oil) at 0° C. under nitrogen atmosphere. The resulting mixture was stirred for 30 min at 0° C. under nitrogen atmosphere. To the above mixture was added CH₃I (0.99 g, 6.97 mmol) at 0° C. The resulting mixture was stirred for an additional 16 h at rt. On completion, the reaction was quenched by the addition of brine (50 mL) and was extracted with EtOAc (3×200 mL). The combined organic layers were washed with brine (2×100 mL), and dried over anhydrous Na₂SO₄. After filtration, the filtrate was concentrated under reduced pressure. The residue was purified by silica gel column chromatography, eluted with PE/EA (18%), to afford the title compound (1.45 g, 92% yield) as a yellow solid. LC/MS (ESI, m/z): [(M+H)]f=272.0, 274.0.

Step 3—2-Bromo-6-[(3R)-3-methoxyoxolan-3-yl]-4-methylpyridine and 2-bromo-6-[(3S)-3-methoxyoxolan-3-yl]-4-methylpyridine. 2-Bromo-6-(3-methoxyoxolan-3-yl)-4-methylpyridine (1.45 g, 5.33 mmol) was purified by Prep-Chiral-HPLC (Column: CHIRAL ART Cellulose-SC, 2*25 cm, 5 μm; Mobile Phase A: Hex (0.5% 2M NH₃-MeOH)-HPLC, Mobile Phase B: IPA: DCM=1: 1-HPLC; Flow rate: 20 mL/min; Gradient: 10% B to 10% B in 8 min; Wave Length: 220/254 nm; RT1(min): 5.82; RT2(min): 6.64). The fractions was collected at 5.82 min (faster eluting peak) and concentrated under vacuum to afford 2-bromo-6-[(3R)-3-methoxyoxolan-3-yl]-4-methylpyridine (414 mg, 29% yield) as a white oil. The fractions were collected at 6.64 min (slower eluting peak) and concentrated under vacuum to afford 2-bromo-6-[(3S)-3-methoxyoxolan-3-yl]-4-methylpyridine (447 mg, 31% yield) as a white oil. LC/MS (ESI, m/z): [(M+H)]⁺=272.0, 274.0 for both isomers. The absolute stereochemistry was assigned arbitrarily. K6-Chloro-N-((1R,2R)-2-methoxycyclobutyl)imidazo[1,2-b]pyridazine-3-carboxamide (Intermediate TE)

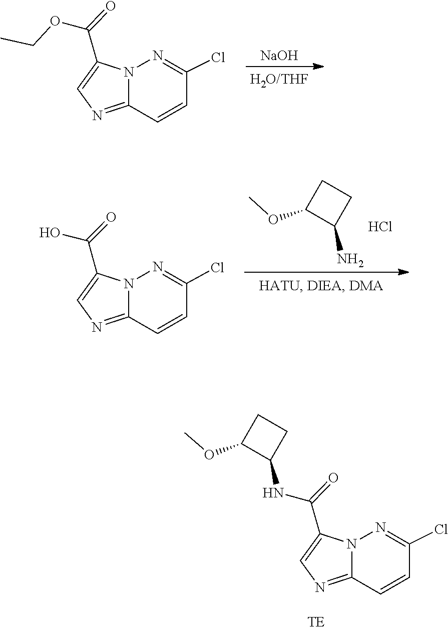

Step 1—6-Chloroimidazo[1,2-b]pyridazine-3-carboxylic acid. To a stirred solution of ethyl 6-chloroimidazo[1,2-b]pyridazine-3-carboxylate (1.00 g, 4.43 mmol, CAS #1150566-27-0) in THF (10 mL) and H₂O (10 mL) was added NaOH (709 mg, 17.7 mmol) in portions at rt under nitrogen atmosphere. The resulting mixture was stirred for 2 h at 60° C. under nitrogen atmosphere. On completion, the mixture was cooled to rt and acidified to pH 5 with HCl (aq.). The precipitated solids were collected by filtration and washed with THF (3×30 mL) and dried to give the title compound (630 mg, 72% yield) as an off-white solid. LC/MS (ESI, m/z): [(M+H)]⁺=198.1.

Step 2—6-Chloro-N-((1R,2R)-2-methoxycyclobutyl)imidazo[1,2-b]pyridazine-3-carboxamide. To a stirred solution of 6-chloroimidazo[1,2-b]pyridazine-3-carboxylic acid (300 mg, 1.52 mmol) and (1R,2R)-2-methoxycyclobutan-1-amine hydrochloride (209 mg, 1.52 mmol, CAS #1820576-22-4) in DMA (10 mL) were added DIEA (589 mg, 4.55 mmol) and HATU (866 mg, 2.28 mmol) at 0° C. under nitrogen atmosphere. The resulting mixture was stirred for 1 h at rt under nitrogen atmosphere. After completion of the reaction, the reaction mixture was purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 30%-50% B in 25 min; Flow rate: 80 mL/min; Detector: 220/254 nm; desired fractions were collected at 43% B and concentrated under reduced pressure) to afford the title compound (350 mg, 82% yield) as a yellow solid. LC/MS (ESI, m/z): [(M+H)]⁺=280.9.

### 6-(Indolin-4-yl)nicotinaldehyde (Intermediate TF)

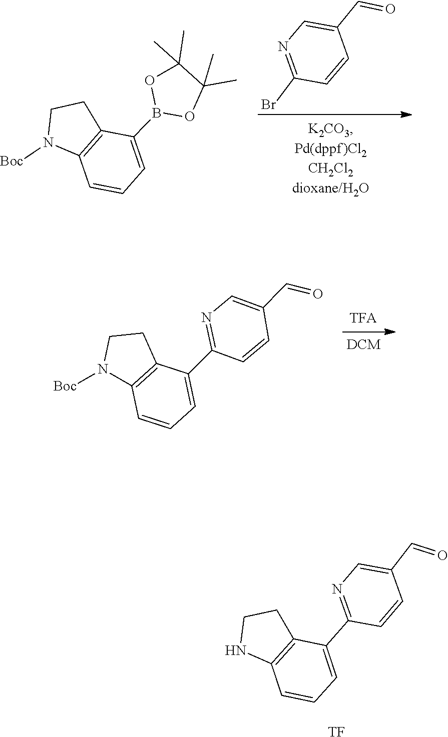

Step 1—Tert-butyl 4-(5-formylpyridin-2-yl)-2,3-dihydroindole-1-carboxylate. To a stirred mixture of tert-butyl 4-(4,4,5,5-tetramethyl-1,3,2-dioxaborolan-2-yl)-2,3-dihydroindole-1-carboxylate (20 g, 60 mmol, synthesized via Step 1 of Intermediate RQ) and 6-bromopyridine-3-carbaldehyde (10.56 g, 56.77 mmol) in 1,4-dioxane (200 mL) and H₂O (40 mL) were added K₂CO₃ (24.02 g, 173.8 mmol) and Pd(dppf)Cl₂ CH₂Cl₂ (4.72 g, 5.79 mmol) at rt under nitrogen atmosphere. The resulting mixture was stirred for 1 h at 90° C. under nitrogen atmosphere. On completion, the mixture was cooled to rt and diluted with water (300 mL) and extracted with EtOAc (3×200 mL). The combined organic layers were washed with water (2×200 mL), and dried over anhydrous MgSO₄. After filtration, the filtrate was concentrated under reduced pressure. The residue was purified by silica gel column chromatography, eluted with PE/EA (7:1), to afford the title compound (14 g, 75% yield) as a white solid. LC/MS (ESI, m/z): [(M+1)]⁺=325.2.

Step 2—6-(2,3-Dihydro-1H-indol-4-yl)pyridine-3-carbaldehyde. To a stirred mixture of tert-butyl 4-(5-formylpyridin-2-yl)-2,3-dihydroindole-1-carboxylate (7 g, 20 mmol) in DCM (60 mL) and TFA (20 mL) at rt. The resulting mixture was stirred for 1 h at rt. On completion, the mixture was concentrated under reduced pressure. The resulting mixture was diluted with water (50 mL) and neutralized to pH 8 with saturated Na₂CO₃ (aq.). The aqueous layer was extracted with EtOAc (3×100 mL). The combined organic layers and dried over anhydrous Na₂SO₄. After filtration, the filtrate was concentrated under reduced pressure to give the title compound (4.8 g, 99% yield) as a light yellow solid. LC/MS (ESI, m/z): [(M+1)]⁺=225.2.

### 6-(4-(5-Formylpyridin-2-yl)indolin-1-yl)-N-((1R,2R)-2-methoxycyclobutyl)imidazo[1,2-b]pyridazine-3-carboxamide (Intermediate TG)

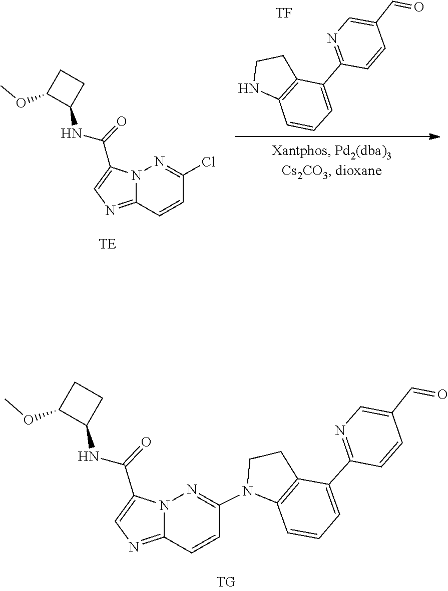 [SMILES: CO[C@@H]1CC[C@H]1NC(=O)c1cnc2ccc(Cl)nn12 ; O=Cc1ccc(-c2cccc3c2CCN3)nc1 ; "TF" ; CO[C@@H]1CC[C@H]1NC(=O)c1cnc2ccc(N3CCc4c(-c5ccc(C=O)cn5)cccc43)nn12]

To a stirred solution of 6-chloro-N-((1R,2R)-2-methoxycyclobutyl)imidazo[1,2-b]pyridazine-3-carboxamide (350 mg, 1.247 mmol, Intermediate TE) and 6-(indolin-4-yl)nicotinaldehyde 419 mg, 1.871 mmol, Intermediate TF) in dioxane (10 mL) were added Cs₂CO₃ (1.22 g, 3.74 mmol) and XantPhos (144 mg, 0.249 mmol) and Pd₂(dba)₃ (228 mg, 0.249 mmol) in turns at rt under nitrogen atmosphere. The resulting mixture was stirred for 2 h at 100° C. under nitrogen atmosphere. On completion, the mixture was concentrated under reduced pressure. The crude product was purified by silica gel column chromatography (Mobile Phase A: Petroleum ether, Mobile Phase B: EtOAc; Gradient: 60% B to 100% B in 20 min, 254 nm; the fractions containing the desired product were collected at 100% B) to afford the title compound (200 mg, 34% yield) as a yellow solid. LC/MS (ESI, m/z): [(M+H)]⁺=469.2.

### 1-[1-(2,6-Dioxopiperidin-3-yl)-3-methyl-2-oxo-1,3-benzodiazol-5-yl]piperidine-4-carbaldehyde (Intermediate TH)

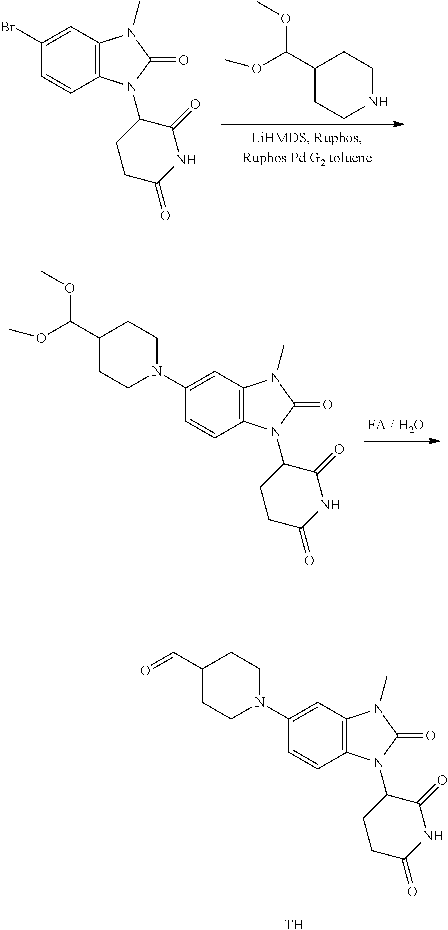

Step 1—3-{5-[4-(Dimethoxymethyl)piperidin-1-yl]-3-methyl-2-oxo-1,3-benzodiazol-1-yl}piperidine-2,6-dione. To a stirred mixture of 3-(5-bromo-3-methyl-2-oxo-1,3-benzodiazol-1-yl)piperidine-2,6-dione (500 mg, 1.48 mmol, CAS #2300099-98-1) and 4-(dimethoxymethyl)piperidine (471 mg, 2.96 mmol) in toluene (10 mL) were added RuPhos (138 mg, 0.296 mmol) and RuPhos-PdCl-2nd G (230 mg, 0.296 mmol) at rt under nitrogen atmosphere. To the above mixture was added LiHMDS (12 mL, 12 mmol). The resulting mixture was stirred for additional 1 h at 80° C. On completion, the mixture was cooled to rt and neutralized to pH 7 with cone. FA. The resulting mixture was concentrated under reduced pressure. The residue was purified by silica gel column chromatography, eluted with CH₂Cl₂/EA (1:1) to afford the title compound (550 mg, 89% yield) as a yellow solid. LC/MS (ESI, m/z): [(M+H)]⁺=417.2.

Step 2—1-[1-(2,6-Dioxopiperidin-3-yl)-3-methyl-2-oxo-1,3-benzodiazol-5-yl]piperidine-4-carbaldehyde. A solution of 3-{5-[4-(dimethoxymethyl)piperidin-1-yl]-3-methyl-2-oxo-1,3-benzodiazol-1-yl}piperidine-2,6-dione (550 mg, 1.32 mmol) in formic acid (3 mL) and H₂O (3 mL) was stirred for 1 h at 50° C. under nitrogen atmosphere. On completion, the mixture was cooled to rt and neutralized to pH 7 with saturated Na₂CO₃ (aq.). The resulting mixture was extracted with EtOAc (3×300 mL). The combined organic layers were dried over anhydrous Na₂SO₄. After filtration, the filtrate was concentrated under reduced pressure to afford the title compound (480 mg, 98% yield) as a white solid. LC/MS (ESI, m/z): [(M+H)]⁺=371.2.

### N-(3-{6-[(3R)-3-methoxyoxolan-3-yl]-4-methylpyridin-2-yl}-1-[2-(piperidin-4-yl)ethyl]pyrrolo[2,3-c]pyridin-5-yl)acetamide trifluoroacetate (Intermediate TI)

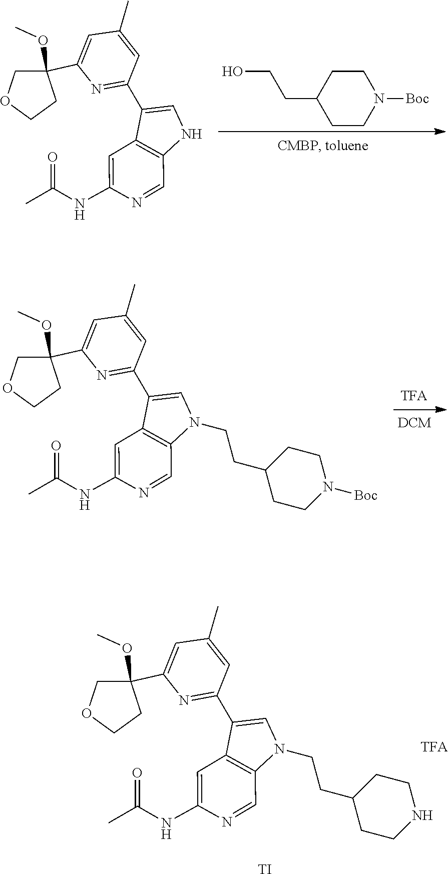 [SMILES: CO[C@@]1(c2cc(C)cc(-c3c[nH]c4cnc(NC(C)=O)cc34)n2)CCOC1 ; CC(C)(C)OC(=O)N1CCC(CCO)CC1 ; CO[C@@]1(c2cc(C)cc(-c3cn(CCC4CCN(C(=O)OC(C)(C)C)CC4)c4cnc(NC(C)=O)cc34)n2)CCOC1 ; CO[C@@]1(c2cc(C)cc(-c3cn(CCC4CCNCC4)c4cnc(NC(C)=O)cc34)n2)CCOC1 ; "TI"]

Step 1—Tert-butyl 4-[2-(5-acetamido-3-{6-[(3R)-3-methoxyoxolan-3-yl]-4-methylpyridin-2-yl}pyrrolo[2,3-c]pyridin-1-yl)ethyl]piperidine-1-carboxylate. To a stirred mixture of N-(3-{6-[(3R)-3-methoxyoxolan-3-yl]-4-methylpyridin-2-yl}-1H-pyrrolo[2,3-c]pyridin-5-yl)acetamide (570 mg, 1.56 mmol, synthesized via Steps 1-2 of Intermediate RN) and tert-butyl 4-(2-hydroxyethyl)piperidine-1-carboxylate (356 mg, 1.55 mmol, CAS #89151-44-0) in toluene (10 mL) was added cyanomethylenetributylphosphorane (563 mg, 2.33 mmol) at rt under nitrogen atmosphere. The resulting mixture was stirred for 3 h at 100° C. under nitrogen atmosphere. On completion, the mixture was cooled to rt and concentrated under reduced pressure. The residue was purified by reverse phase flash chromatography (Column: Spherical C18, 20-40 μm, 330 g; Mobile Phase A: Water (plus 10 mM NH₄HCO₃); Mobile Phase B: ACN; Flow rate: 80 mL/min; Gradient: 60% B-80% B in 20 min; Detector: 254 nm; the fractions containing desired product were collected at 69% B and concentrated under reduced pressure) to afford the title compound (820 mg, 91% yield) as a light yellow solid. LC/MS (ESI, m/z): [(M+H)]⁺=578.3.

Step 2—N-(3-{6-[(3R)-3-methoxyoxolan-3-yl]-4-methylpyridin-2-yl}-1-[2-(piperidin-4-yl)ethyl]pyrrolo[2,3-c]pyridin-5-yl)acetamide trifluoroacetate. To a stirred solution of tert-butyl 4-[2-(5-acetamido-3-{6-[(3R)-3-methoxyoxolan-3-yl]-4-methylpyridin-2-yl}pyrrolo[2,3-c]pyridin-1-yl)ethyl]piperidine-1-carboxylate (800 mg, 1.39 mmol) in DCM (4 mL) was added TFA (4 mL) at rt under nitrogen atmosphere and the mixture was stirred for 1 h at rt under nitrogen atmosphere. On completion, the mixture was concentrated under reduced pressure. The residue was purified by trituration with ethyl acetate (30 mL) to afford the title compound (750 mg) as a light green solid. LC/MS (ESI, m/z): [(M+H)]f=478.2.

### 4-Chloro-3-(2,4-dioxo-1,3-diazinan-1-yl)-5-fluorobenzoic acid (Intermediate TJ)

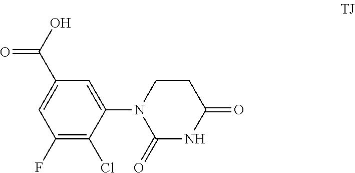 [SMILES: O=C1CCN(c2cc(C(=O)O)cc(F)c2Cl)C(=O)N1]

### 4-(5-(1,3-Dioxolan-2-yl)pyridin-2-yl)-6-fluoroindoline (Intermediate TK)

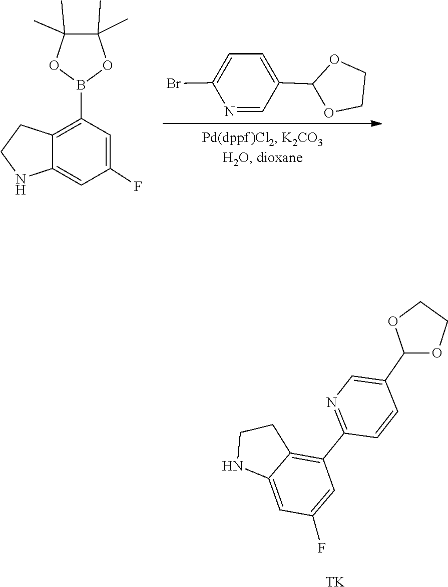

To a stirred solution of 6-fluoro-4-(4,4,5,5-tetramethyl-1,3,2-dioxaborolan-2-yl)indoline (1.80 g, 6.84 mmol, synthesized via Step 1 of Intermediate MX) and 2-bromo-5-(1,3-dioxolan-2-yl)pyridine (1.57 g, 6.84 mmol) in H₂O (4 mL) and dioxane (20 mL) were added K₂CO₃ (2.83 g, 20.5 mmol) and Pd(dppf)Cl₂·CH₂Cl₂ (0.56 g, 0.684 mmol) at rt under nitrogen atmosphere. The resulting mixture was stirred for 2 h at 90° C. under nitrogen atmosphere. On completion, the mixture was cooled to rt and concentrated under vacuum. The residue was purified by silica gel column chromatography, eluted with petroleum ether/EtOAc (3:1), to afford the title compound (1.4 g, 71% yield) as a yellow solid. LC/MS (ESI, m/z): [(M+1)]=287.1.

### 1-{2-Chloro-5-[4-(piperidin-4-ylmethyl)piperazine-1-carbonyl]phenyl}-1,3-diazinane-2,4-dione trifluoroacetate (Intermediate TL)

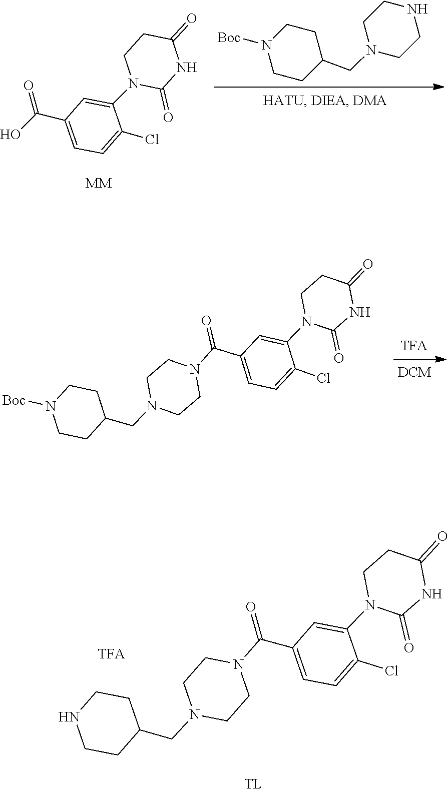

Step 1—Tert-butyl 4-({4-[4-chloro-3-(2,4-dioxo-1,3-diazinan-1-yl)benzoyl]piperazin-1-yl}methyl)piperidine-1-carboxylate. To a stirred mixture of 4-chloro-3-(2,4-dioxo-1,3-diazinan-1-yl)benzoic acid (200 mg, 0.744 mmol, Intermediate MM) and tert-butyl 4-(piperazin-1-ylmethyl)piperidine-1-carboxylate (211 mg, 0.744 mmol, CAS #381722-48-1) in DMA (4 mL) were added DIEA (289 mg, 2.236 mmol) and HATU (424 mg, 1.12 mmol) at rt under nitrogen atmosphere. The resulting mixture was stirred for 1 h at rt under nitrogen atmosphere. On completion, the mixture was concentrated under reduced pressure. The residue was purified by reverse phase flash chromatography (Column: WelFlash™ C18-I, 20-40 μm, 120 g; Eluent A: Water (plus 10 mmol/L FA); Eluent B: ACN; Gradient: 15%-40% B in 25 min; Flow rate: 50 mL/min; Detector: 254 nm; desired fractions were collected at 27% B and concentrated under reduced pressure) to afford the title compound (380 mg, 96% yield) as a white oil. LC/MS (ESI, m/z): [(M+H)]f=534.2.

Step 2—1-{2-Chloro-5-[4-(piperidin-4-ylmethyl)piperazine-1-carbonyl]phenyl}-1,3-diazinane-2,4-dione trifluoroacetate. To a stirred solution of tert-butyl 4-({4-[4-chloro-3-(2,4-dioxo-1,3-diazinan-1-yl)benzoyl]piperazin-1-yl}methyl)piperidine-1-carboxylate (350 mg, 0.655 mmol) in DCM (4 mL) was added TFA (2 mL) at rt under nitrogen atmosphere and the mixture was stirred for 1 h at rt under nitrogen atmosphere. On completion, the mixture was concentrated under reduced pressure. The residue was purified by trituration with ethyl ether (10 mL) to afford the title compound (300 mg) as a white solid. LC/MS (ESI, m/z): [(M+H)]⁺=434.1.

### 1-{2-Chloro-5-[4-({3′,3′-difluoro-[1,4′-bipiperidin]-4-yl}methyl)piperazine-1-carbonyl]phenyl}-1,3-diazinane-2,4-dione trifluoroacetate (Intermediate™)

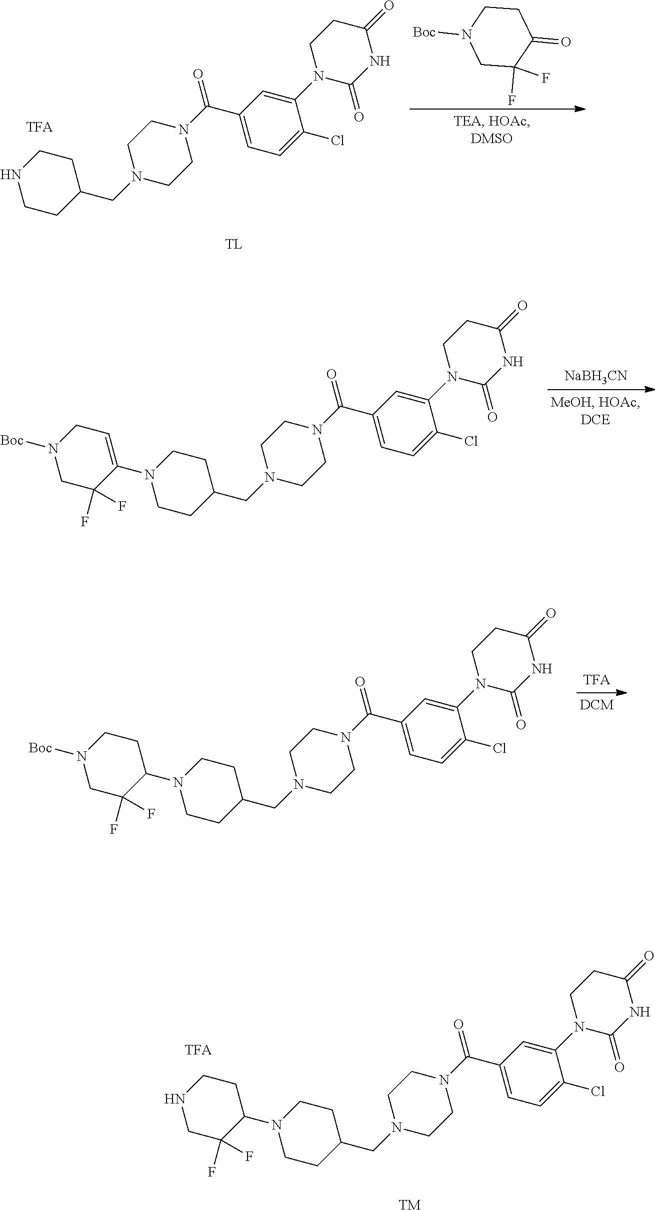

Step 1—Tert-butyl 4-[4-({4-[4-chloro-3-(2,4-dioxo-1,3-diazinan-1-yl)benzoyl]piperazin-1-yl}methyl)piperidin-1-yl]-3,3-difluoro-2,6-dihydropyridine-1-carboxylate. To a stirred mixture of 1-{2-chloro-5-[4-(piperidin-4-ylmethyl)piperazine-1-carbonyl]phenyl}-1,3-diazinane-2,4-dione trifluoroacetate (200 mg, 0.376 mmol, Intermediate TL) and TEA (0.16 mL, 1.13 mmol) in DMSO (5 mL) were added tert-butyl 3,3-difluoro-4-oxopiperidine-1-carboxylate (88 mg, 0.37 mmol) and HOAc (0.11 mL, 1.88 mmol) at rt under nitrogen atmosphere. The resulting mixture was stirred for 16 h at 100° C. under nitrogen atmosphere. On completion, the mixture was cooled to rt and diluted with water (20 mL). The resulting mixture was extracted with EtOAc (3×50 mL). The combined organic phase was washed with brine (1×100 mL), dried with Na₂SO₄ and filtered. The filtrate was concentrated under reduced pressure to give the title compound (300 mg) as a yellow solid. LC/MS (ESI, m/z): [(M+H)]⁺=651.3.

Step 2—Tert-butyl 4-({4-[4-chloro-3-(2,4-dioxo-1,3-diazinan-1-yl)benzoyl]piperazin-1-yl}methyl)-3′,3′-difluoro-[1,4′-bipiperidine]-1′-carboxylate. To a stirred solution of tert-butyl 4-[4-({4-[4-chloro-3-(2,4-dioxo-1,3-diazinan-1-yl)benzoyl]piperazin-1-yl}methyl)piperidin-1-yl]-3,3-difluoro-2,6-dihydropyridine-1-carboxylate (270 mg, 0.415 mmol) in HOAc (0.3 mL), DCE (3 mL) and MeOH (3 mL) was added NaBH₃CN (78 mg, 1.2 mmol) at rt under nitrogen atmosphere and the mixture was stirred for 2 h at rt under nitrogen atmosphere. On completion, the mixture was concentrated under reduced pressure. The reside was purified by reverse phase flash chromatography (Column: WelFlash™ C18-I, 20-40 μm, 330 g; Eluent A: Water (plus 10 mmol/L NH₄HCO₃); Eluent B: ACN; Gradient: 40%-60% B in 15 min; Flow rate: 80 mL/min; Detector: 220/254 nm; desired fractions were collected at 50% B and concentrated under reduced pressure) to afford the title compound (150 mg, 55% yield) as a white solid. LC/MS (ESI, m/z): [(M+H)]f=653.3.

Step 3—1-{2-Chloro-5-[4-({3′,3′-difluoro-[1,4′-bipiperidin]-4-yl}methyl)piperazine-1-carbonyl]phenyl}-1,3-diazinane-2,4-dione trifluoroacetate. To a stirred solution of tert-butyl 4-({4-[4-chloro-3-(2,4-dioxo-1,3-diazinan-1-yl)benzoyl]piperazin-1-yl}methyl)-3′,3′-difluoro-[1,4′-bipiperidine]-1′-carboxylate (90 mg, 0.14 mmol) in DCM (1 mL) was added TFA (1 mL) at rt under nitrogen atmosphere and the mixture was stirred for 1 h. On completion, the mixture was concentrated under reduced pressure. The residue was purified by trituration with ethyl acetate (30 mL) to give the title compound (80 mg, 89% yield) as a white solid. LC/MS (ESI, m/z): [(M+H)]⁺=553.5.

### Tert-butyl 4-iodo-2,3-dihydroindole-1-carboxylate (Intermediate TN)

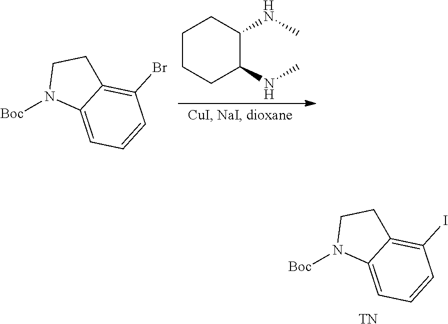

To a stirred mixture of tert-butyl 4-bromo-2,3-dihydroindole-1-carboxylate (20 g, 67 mmol, synthesized via Step 1 of Intermediate OK) and (1S,2S)—N1,N2-dimethylcyclohexane-1,2-diamine (9.54 g, 67.1 mmol) in 1,4-dioxane (200 mL) were added CuI (0.64 g, 3.35 mmol) and NaI (1.01 g, 6.71 mmol) at rt under nitrogen atmosphere. The resulting mixture was stirred for 3 days at 110° C. under nitrogen atmosphere. On completion, the mixture was cooled to rt and concentrated under vacuum. The residue was purified by silica gel column chromatography, eluted with PE/EA (1:1), to afford the title compound (15 g, 65% yield) as a brown solid. LC/MS (ESI, m/z): [(M+H)]f=346.0.

### [3-(2,3-Dihydro-1H-indol-4-yl)bicyclo[1.1.1]pentan-1-yl]methanol (Intermediate TO)

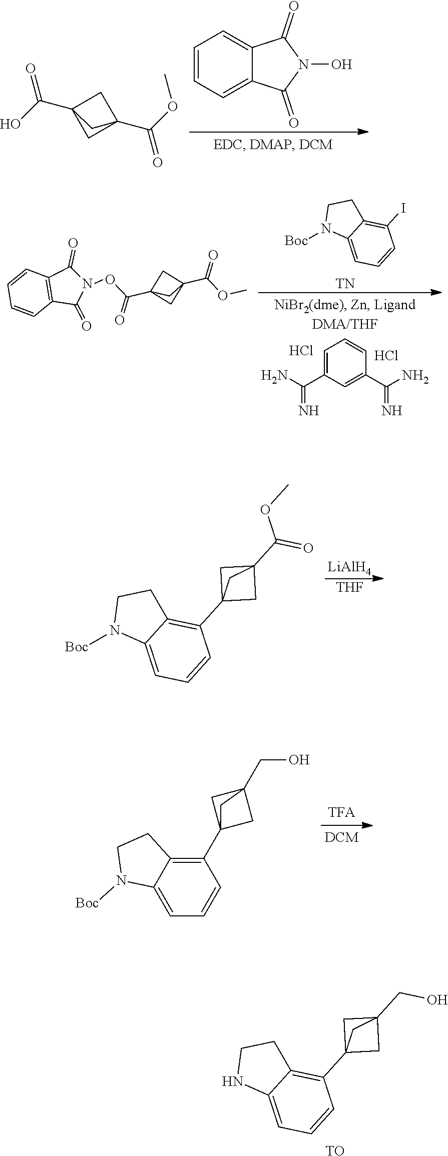

Step 1—1-(1,3-Dioxoisoindol-2-yl) 3-methyl bicyclo[1.1.1]pentane-1,3-dicarboxylate. To a stirred mixture of 3-(methoxycarbonyl)bicyclo[1.1.1]pentane-1-carboxylic acid (15 g, 88 mmol, CAS #83249-10-9) in DCM (250 mL) were added DMAP (2.15 g, 17.6 mmol) and N-hydroxyphthalimide (14.38 g, 88.15 mmol) at 0° C. under nitrogen atmosphere. The resulting mixture was stirred for 0.5 h at 0° C. under nitrogen atmosphere. To the above mixture was added EDC-HCl (33.80 g, 176.3 mmol) at 0° C. The resulting mixture was stirred for an additional 2 h at 0° C. The resulting mixture was diluted with water (200 mL) and extracted with CH₂Cl₂ (3×200 mL). The combined organic layers were dried over anhydrous Na₂SO₄, filtered, and the filtrate was concentrated under reduced pressure. The residue was purified by silica gel column chromatography, eluted with PE/EA (1:1), to afford the title compound (12.5 g, 45% yield) as a white solid.

Step 2—Tert-butyl 4-[3-(methoxycarbonyl)bicyclo[1.1.1]pentan-1-yl]-2,3-dihydroindole-1-carboxylate. To a mixture of 1-(1,3-dioxoisoindol-2-yl) 3-methylbicyclo[1.1.1]pentane-1,3-dicarboxylate (3 g, 10 mmol), tert-butyl 4-iodo-2,3-dihydroindole-1-carboxylate (3.28 g, 9.52 mmol, Intermediate TN) and Zn (9.33 g, 143 mmol) was added a solution of pyridine-2,6-dicarboximidamide dihydrochloride (0.45 g, 1.9 mmol) and NiBr₂·DME (0.67 g, 1.9 mmol) in DMA (25 mL) and THF (25 mL) at rt under argon atmosphere. The resulting mixture was stirred for 16 h at rt under argon atmosphere. On completion, the mixture was filtered, and the filter cake was washed with ACN (5×10 mL). The filtrate was concentrated under reduced pressure and the residue was diluted with water (300 mL). The resulting mixture was extracted with EtOAc (3×500 mL). The combined organic layers were washed with brine (1×500 mL), dried over anhydrous Na₂SO₄, filtered, and the filtrate was concentrated under reduced pressure. The residue was purified by silica gel column chromatography, eluted with PE/EA (5:1), to afford the title compound (1.2 g, 37% yield) as a white solid. LC/MS (ESI, m/z): [(M+H)]⁺=344.1.

Step 3—Tert-butyl 4-[3-(hydroxymethyl)bicyclo[1.1.1]pentan-1-yl]-2,3-dihydroindole-1-carboxylate. To a stirred solution of tert-butyl 4-[3-(methoxycarbonyl)bicyclo[1.1.1]pentan-1-yl]-2,3-dihydroindole-1-carboxylate (600 mg, 2 mmol) in THE (20 mL) was added LiAlH₄ (3.49 mL, 3.49 mmol) at 0° C. under nitrogen atmosphere. The resulting mixture was stirred for 10 min at rt under nitrogen atmosphere. On completion, the reaction was quenched by the addition of sat. NH4Cl (aq.) at 0° C. The resulting mixture was extracted with EtOAc (3×50 mL). The combined organic layers were washed with brine (2×50 mL), dried over anhydrous Na₂SO₄, filtered, and the filtrate was concentrated under reduced pressure. The residue was purified by silica gel column chromatography, eluted with PE/EA (3:1), to afford the title compound (400 mg, 73% yield) as a brown oil. LC/MS (ESI, m/z): [(M+H)]f=316.1.

Step 4-[3-(2,3-dihydro-1H-indol-4-yl)bicyclo[1.1.1]pentan-1-yl]methanol. To a stirred solution of tert-butyl 4-[3-(hydroxymethyl)bicyclo[1.1.1]pentan-1-yl]-2,3-dihydroindole-1-carboxylate (400 mg, 1 mmol) in DCM (5 mL) was added TFA (2 mL) at rt under nitrogen atmosphere. The resulting mixture was stirred for 10 min at rt under nitrogen atmosphere. On completion, the mixture was basified to pH 9 with saturated Na₂CO₃ (aq.). The resulting mixture was extracted with EtOAc (3×50 mL). The combined organic layers were washed with brine (1×50 mL), dried over anhydrous Na₂SO₄, filtered, and the filtrate was concentrated under reduced pressure. The residue was purified by silica gel column chromatography, eluted with PE/EA (1:1), to afford the title compound (220 mg, 81% yield) as a yellow oil. LC/MS (ESI, m/z): [(M+H)]⁺=216.1.

### N-[(1R,2S)-2-fluorocyclopropyl]-8-(methylamino)-6-[4-(3-{[4-(piperidin-4-yl)piperazin-1-yl]methyl}bicyclo[1.1.1]pentan-1-yl)-2,3-dihydroindol-1-yl]imidazo[1,2-b]pyridazine-3-carboxamide trifluoroacetate (Intermediate TP)

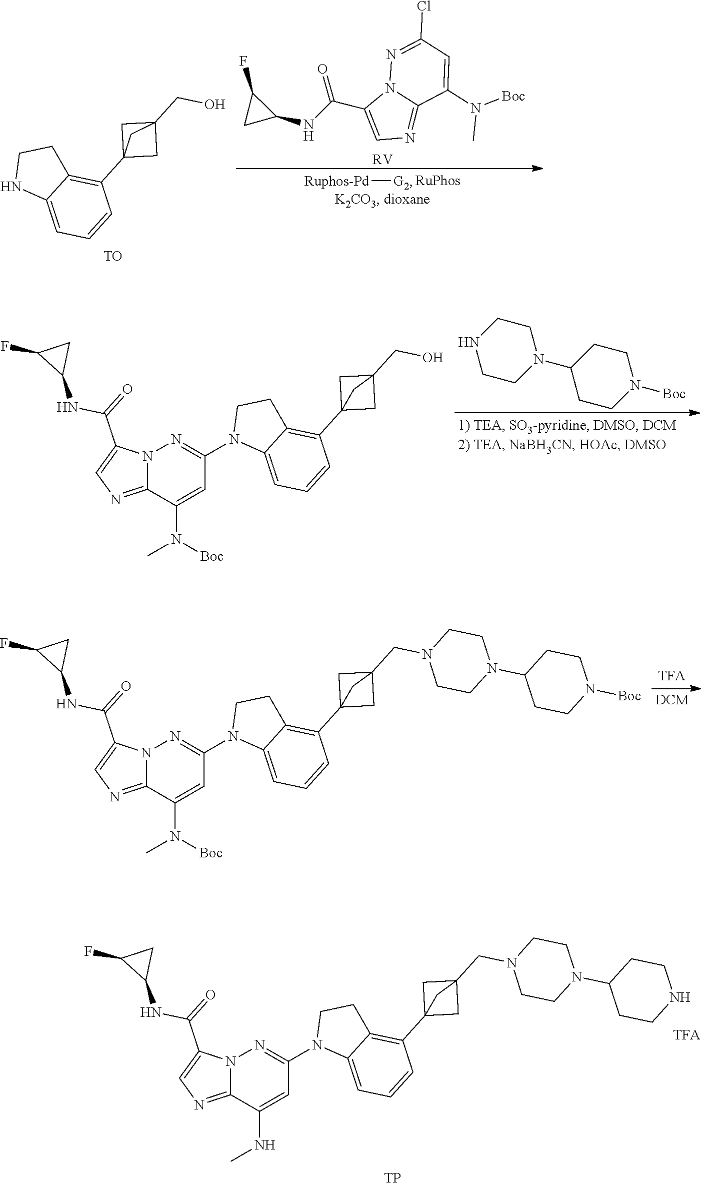

Step 1—Tert-butyl N-(3-{[(1R,2S)-2-fluorocyclopropyl]carbamoyl}-6-{4-[3-(hydroxymethyl)bicyclo[1.1.1]pentan-1-yl]-2,3-dihydroindol-1-yl}imidazo[1,2-b]pyridazin-8-yl)-N-methylcarbamate. To a stirred mixture of [3-(2,3-dihydro-1H-indol-4-yl)bicyclo[1.1.1]pentan-1-yl]methanol (220 mg, 1.02 mmol. Intermediate TO) and tert-butyl N-(6-chloro-3-{[(1S,2R)-2-fluorocyclopropyl]carbamoyl}imidazo[1,2-b]pyridazin-8-yl)-N-methylcarbamate (392.20 mg, 1.022 mmol, Intermediate RV) in 1,4-dioxane (20 mL) were added K₂CO₃ (423.67 mg, 3.066 mmol). RuPhos-PdCl-2nd G (158.95 mg, 0,204 mmol) and RuPhos (2.17 mg, 0,005 mmol) at rt under nitrogen atmosphere. The resulting mixture was stirred for 1 h at 90° C. under nitrogen atmosphere. On completion, the mixture was cooled to rt and concentrated under vacuum. The residue was purified by silica gel column chromatography, eluted with CH₂CH₂/EA (1:1), to afford the title compound (310 mg, 54% yield) as a yellow solid. LC/MS (ESI, m/z): [(M+H)]⁺=563.2.

Step 2—Tert-butyl 4-(4-{[3-(1-{8-[(tert-butoxycarbonyl)(methyl)amino]-3-{[(1R,2S)-2-fluorocyclopropyl]carbamoyl}imidazo[1,2-b]pyridazin-6-yl}-2,3-dihydroindol-4-yl)bicyclo[1.1.1]pentan-1-yl]methyl}piperazin-1-yl)piperidine-1-carboxylate. To a stirred solution of tert-butyl N-(3-{[(1R,2S)-2-fluorocyclopropyl]carbamoyl}-6-{4-[3-(hydroxymethyl)bicyclo[1.1.1]pentan-1-yl]-2,3-dihydroindol-1-yl}imidazo[1,2-b]pyridazin-8-yl)-N-methylcarbamate (100 mg, 0.178 mmol) in DMSO (3 mL) and DCM (1 mL) was added TEA (0.2 mL, 1.4 mmol) at 0° C. under nitrogen atmosphere. To the above mixture was added SO₃-pyridine (113.15 mg, 0.712 mmol) at 0° C. The resulting mixture was stirred for 1 h at rt. After full conversion from alcohol to aldehyde, to the above mixture were added tert-butyl 4-(piperazin-1-yl)piperidine-1-carboxylate (38.44 mg, 0.143 mmol) and AcOH (0.06 mL, 1.07 mmol) at rt. The resulting mixture was stirred for an additional 1 h at 50° C. The mixture was allowed to cool down to rt. To the above mixture was added NaBH₃CN (17.93 mg, 0.286 mmol) and the mixture was stirred for additional 16 h at 50° C. on completion, the mixture was cooled to rt and concentrated under vacuum. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 65%-95% B in 25 min; Flow rate: 60 mL/min; Detector: 220/254 nm; desired fractions were collected at 85% B and concentrated under reduced pressure) to afford the title compound (60 mg, 42% yield) as a yellow solid. LC/MS (ESI, m/z): [(M+H)]=814.4.

Step 3—N-[(1R,2S)-2-fluorocyclopropyl]-8-(methylamino)-6-[4-(3-{[4-(piperidin-4-yl)piperazin-1-yl]methyl}bicyclo[1.1.1]pentan-1-yl)-2,3-dihydroindol-1-yl]imidazo[1,2-b]pyridazine-3-carboxamide trifluoroacetate. To a stirred solution of tert-butyl 4-(4-{[3-(1-{8-[(tert-butoxycarbonyl)(methyl)amino]-3-{[(1R,2S)-2-fluorocyclopropyl]carbamoyl}imidazo[1,2-b]pyridazin-6-yl}-2,3-dihydroindol-4-yl)bicyclo[1.1.1]pentan-1-yl]methyl}piperazin-1-yl)piperidine-1-carboxylate (60 mg, 0.074 mmol) in DCM (2 mL) was added TFA (1 mL) at rt under nitrogen atmosphere and the mixture was stirred for 1 h at rt under nitrogen atmosphere. On completion, the mixture was concentrated under reduced pressure. The residue was purified by trituration with Et₂O (10 mL) to afford the title compound (50 mg, 95% yield) as a brown solid. LC/MS (ESI, m/z): [(M+H)]⁺=614.3.

### 4-Chloro-3-(2,4-dioxotetrahydropyrimidin-1(2H)-yl)-5-fluorobenzoic acid (Intermediate TQ)

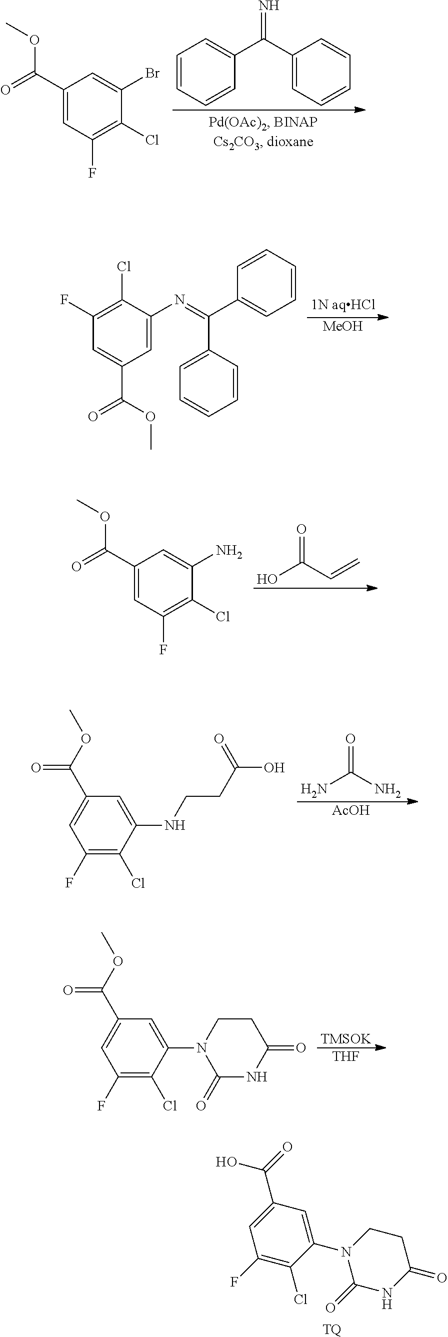

Step 1—Methyl 4-chloro-3-((diphenylmethylene)amino)-5-fluorobenzoate. To a stirred methyl 3-bromo-4-chloro-5-fluorobenzoate (4.00 g, 15.0 mmol) and diphenylmethanimine (2.68 g, 14.8 mmol) in 1,4-dioxane (20 mL) were added Cs₂CO₃ (4.85 g, 14.9 mmol), BINAP (0.93 g, 1.49 mmol) and palladium acetate (0.64 g, 2.84 mmol) at rt under nitrogen atmosphere. The resulting mixture was stirred for 1 h at 100° C. under nitrogen atmosphere. On completion, the mixture was cooled to rt and purified directly by silica gel column chromatography, eluted with petroleum ether/EtOAc (1:1), to afford the title compound (4.80 g, 87% yield) as a white solid. LC/MS (ESI, m/z): [(M+1)]=368.1.

Step 2—Methyl 3-amino-4-chloro-5-fluorobenzoate. To a stirred mixture of methyl 4-chloro-3-[(diphenylmethylidene)amino]-5-fluorobenzoate (4.80 g, 13.1 mmol) in MeOH (25 mL) and 1 NHCl (25 mL) at rt. The resulting mixture was stirred for 2 h at 50° C. On completion, the mixture was cooled to rt and concentrated under reduced pressure. The resulting mixture was diluted with water (100 mL) and was extracted with EtOAc (3×300 mL). The combined organic layers were washed with brine (3×300 mL), and dried over anhydrous Na₂SO₄. After filtration, the filtrate was concentrated under reduced pressure. The residue was purified by silica gel column chromatography, eluted with petroleum ether/EtOAc (3:1) to afford the title compound (1.9 g, 71% yield) as a white solid. LC/MS (ESI, m/z): [(M+1)]=204.0.

Step 3—3-((2-Chloro-3-fluoro-5-(methoxycarbonyl)phenyl)amino)propanoic acid. A solution of methyl 3-amino-4-chloro-5-fluorobenzoate (1.90 g, 9.33 mmol) in acrylic acid (1.34 g, 18.6 mmol) was stirred for 2 h at 100° C. under nitrogen atmosphere. On completion, the mixture was cooled to rt and diluted with water (100 mL). The aqueous layer was extracted with EtOAc (3×100 mL). The combined organic layers and dried over anhydrous Na₂SO₄. After filtration, the filtrate was concentrated under reduced pressure to give the title compound (2.50 g) as a brown solid. LC/MS (ESI, m/z): [(M+1)]⁺=276.0.

Step 4—Methyl 4-chloro-3-(2,4-dioxotetrahydropyrimidin-1(2H)-yl)-5-fluorobenzoate. A mixture of 3-((2-chloro-3-fluoro-5-(methoxycarbonyl)phenyl)amino)propanoic acid (2.50 g, 9.07 mmol) and urea (3.81 g, 63.5 mmol) in AcOH (30 mL) was stirred for 16 h at 110° C. On completion, the mixture was cooled to rt and purified directly by reverse phase Flash chromatography (Column: WelFlash™ C18-I, 20-40 μm, 330 g; Eluent A: Water (plus 10 mmol/L NH₄HCO₃); Eluent B: ACN; Gradient: 35%-55% B in 25 min; Flow rate: 80 mL/min; Detector: 220/254 nm; desired fractions were collected at 42% B and concentrated under reduced pressure) to give the title compound (400 mg, 15% yield) as a white solid. LC/MS (ESI, m/z): [(M+1)]⁺=301.0.

Step 5—4-chloro-3-(2,4-dioxotetrahydropyrimidin-1(2H)-yl)-5-fluorobenzoic acid. To a stirred solution of methyl 4-chloro-3-(2,4-dioxotetrahydropyrimidin-1(2H)-yl)-5-fluorobenzoate (400 mg, 1.330 mmol) in THF (8 mL) was added trimethyl(potassiooxy)silane (853 mg, 6.65 mmol) at rt. The resulting mixture was stirred for 1 h at rt. On completion, the mixture was acidified to pH 5 with conc. HCl. The mixture was purified by reversed-phase flash chromatography (column, C18 silica gel; mobile phase, MeCN in Water (10 mmol/L NH₄HCO₃), 5% to 30% gradient in 30 min; detector, UV 254 nm) to afford the title compound (300 mg, 78% yield) as a white solid. LC/MS (ESI, m/z): [(M+1)]⁺=287.0.

### Tert-butyl 8-(3,3-difluoropiperidin-4-yl)-2,8-diazaspiro[4.5]decane-2-carboxylate (Intermediate TR)

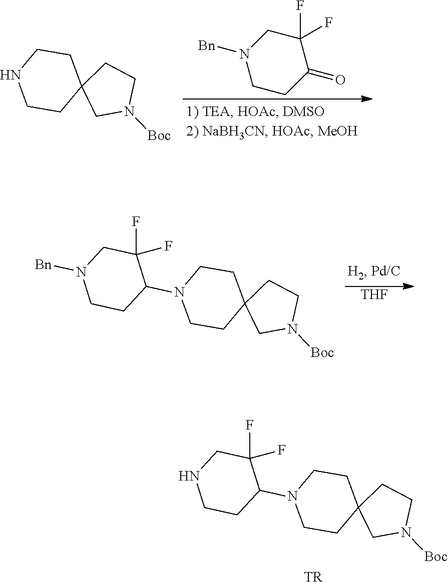 [SMILES: CC(C)(C)OC(=O)N1CCC2(CCNCC2)C1 ; O=C1CCN(Cc2ccccc2)CC1(F)F ; CC(C)(C)OC(=O)N1CCC2(CCN(C3CCN(Cc4ccccc4)CC3(F)F)CC2)C1 ; CC(C)(C)OC(=O)N1CCC2(CCN(C3CCNCC3(F)F)CC2)C1 ; "TR" ; "THF"]

Step 1—Tert-butyl 8-(1-benzyl-3,3-difluoro-1,2,3,6-tetrahydropyridin-4-yl)-2,8-diazaspiro[4.5]decane-2-carboxylate. To a stirred mixture of tert-butyl 2,8-diazaspiro[4.5]decane-2-carboxylate (2.00 g, 8.32 mmol, CAS #336191-17-4) and TEA (3.47 mL, 25.0 mmol) in DMSO (20 mL) were added 1-benzyl-3,3-difluoropiperidin-4-one (3.75 g, 16.6 mmol) and HOAc (2.38 mL, 41.6 mmol) at rt under nitrogen atmosphere. The resulting mixture was stirred for 1 h at 100° C. under nitrogen atmosphere. The mixture was allowed to cool down to rt. To the above mixture were added MeOH (10 mL), NaBH₃CN (1.57 g, 24.963 mmol) and HOAc (1 mL) at rt. The resulting mixture was stirred for 2 h at 50° C. under nitrogen atmosphere. On completion, the mixture was cooled to rt and concentrated under reduced pressure. The residue was purified by reverse phase flash chromatography (Column: WelFlash™ C18-I, 20-40 μm, 320 g; Eluent A: Water (plus 10 mmol/L NH₄HCO₃); Eluent B: ACN; Gradient: 75%-95% B in 15 min; Flow rate: 80 mL/min; Detector: 220/254 nm; desired fractions were collected at 87% B) and concentrated under reduced pressure to afford the title compound (2.00 g, 55% yield) as a yellow oil. LC/MS (ESI, m/z): [(M+H)]⁺=450.3.

Step 2—Tert-butyl 8-(3,3-difluoropiperidin-4-yl)-2,8-diazaspiro[4.5]decane-2-carboxylate. To a stirred solution of tert-butyl 8-(1-benzyl-3,3-difluoropiperidin-4-yl)-2,8-diazaspiro[4.5]decane-2-carboxylate (800 mg, 2 mmol) in THF (10 mL) was added Pd/C (300 mg, 10 wt %) at rt under nitrogen atmosphere. The resulting mixture was purged with hydrogen three times and stirred for 2 h at rt under hydrogen atmosphere. On completion, the mixture was filtered, the filter cake was washed with THF (3×5 mL). The filtrate was concentrated under reduced pressure to give the title compound (630 mg, 99% yield) as a white oil. LC/MS (ESI, m/z): [(M+H)]⁺=360.2.

### 6-{4-[(4-{2,8-diazaspiro[4.5]decan-8-yl}-3,3-difluoropiperidin-1-yl)methyl]-2,3-dihydroindol-1-yl}-N-[(1R,2S)-2-fluorocyclopropyl]-8-(methylamino)imidazo[1,2-b]pyridazine-3-carboxamide trifluoroacetate (Intermediate TS)

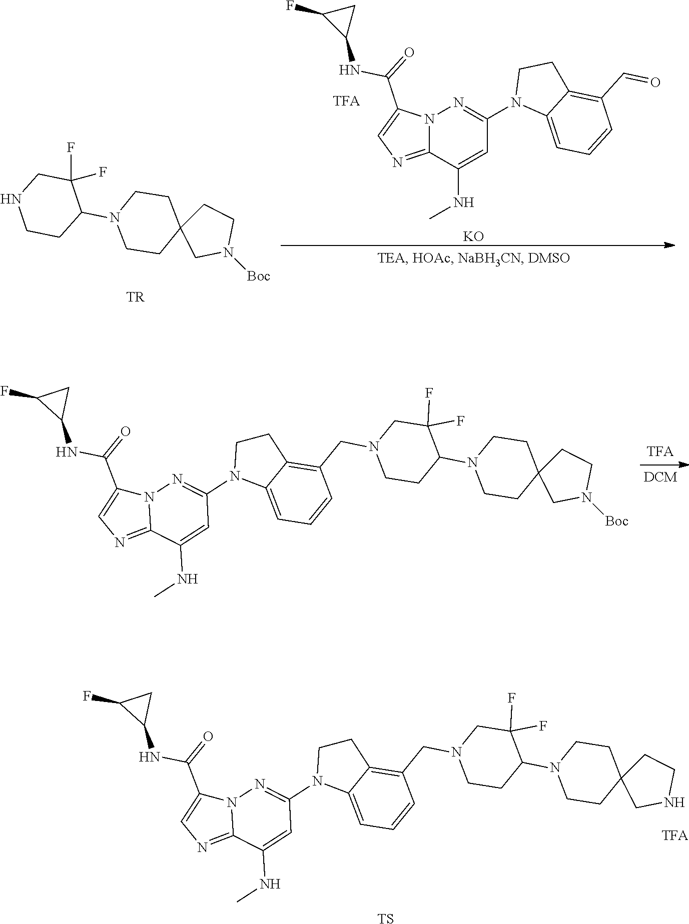

Step 1—Tert-butyl 8-(3,3-difluoro-1-{[1-(3-{[(1R,2S)-2-fluorocyclopropyl]carbamoyl}-8-(methylamino)imidazo[1,2-b]pyridazin-6-yl)-2,3-dihydroindol-4-yl]methyl}piperidin-4-yl)-2,8-diazaspiro[4.5]decane-2-carboxylate. To a stirred mixture of tert-butyl 8-(3,3-difluoropiperidin-4-yl)-2,8-diazaspiro[4.5]decane-2-carboxylate (200 mg, 0.556 mmol, Intermediate TR) and TEA (0.23 mL, 1.67 mmol) in DMSO (3 mL) were added N-[(1R,2S)-2-fluorocyclopropyl]-6-(4-formyl-2,3-dihydroindol-1-yl)-8-(methylamino)imidazo[1,2-b]pyridazine-3-carboxamide trifluoroacetate (272 mg, 0.555 mmol, Intermediate KO) and HOAc (0.16 mL, 2.8 mmol) at rt under nitrogen atmosphere. The resulting mixture was stirred for 30 min at 50° C. under nitrogen atmosphere. The mixture was allowed to cool down to rt. To the above mixture was added NaBH₃CN (105 mg, 1.671 mmol). The resulting mixture was stirred for an additional 2 h at 50° C. On completion, the mixture was cooled to rt and concentrated under reduced pressure. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 35%-55% B in 15 min; Flow rate: 55 mL/min; Detector: 220/254 nm; desired fractions were collected at 50% B and concentrated under reduced pressure) to afford the title compound (190 mg, 46% yield) as a white solid. LC/MS (ESI, m/z): [(M+H)]⁺=738.4.

Step 2—6-{4-[(4-{2,8-Diazaspiro[4.5]decan-8-yl}-3,3-difluoropiperidin-1-yl)methyl]-2,3-dihydroindol-1-yl}-N-[(1R,2S)-2-fluorocyclopropyl]-8-(methylamino)imidazo[1,2-b]pyridazine-3-carboxamide trifluoroacetate. To a stirred solution of tert-butyl 8-(3,3-difluoro-1-{[1-(3-{[(1R,2S)-2-fluorocyclopropyl]carbamoyl}-8-(methylamino)imidazo[1,2-b]pyridazin-6-yl)-2,3-dihydroindol-4-yl]methyl}piperidin-4-yl)-2,8-diazaspiro[4.5]decane-2-carboxylate (190 mg, 0.26 mmol) in DCM (1 mL) was added TFA (1 mL) at rt under nitrogen atmosphere and the mixture was stirred for 1 h at rt. On completion, the mixture was concentrated under reduced pressure. The residue was purified by trituration with Et₂O (10 mL) to afford the title compound (180 mg) as a yellow solid. LC/MS (ESI, m/z): [(M+H)]=638.3. Benzyl 4-(5-bromopyrazin-2-yl)indoline-1-carboxylate (Intermediate TT)

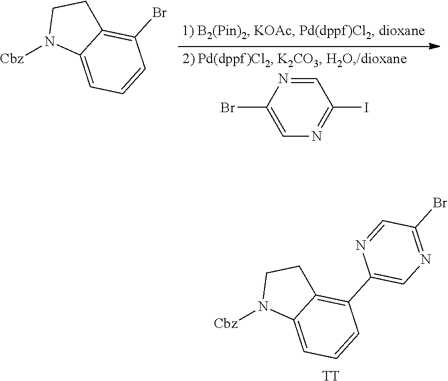

To a stirred mixture of benzyl 4-bromo-2,3-dihydroindole-1-carboxylate (1.5 g, 4.5 mmol, synthesized via Step 1 of Intermediate OK) and bis(pinacolato)diboron (1.15 g, 4.5 mmol) in dioxane (10 mL) were added KOAc (1.33 g, 13.6 mmol) and Pd(dppf)Cl₂CH₂Cl₂(0.74 g, 0.90 mmol) at rt under nitrogen atmosphere. The resulting mixture was stirred for 2 h at 90° C. under nitrogen atmosphere. The mixture was allowed to cool down to rt. To the above mixture were added 2-bromo-5-iodopyrazine (2.25 g, 7.91 mmol), K₂CO₃ (1.64 g, 11.9 mmol), Pd(dppf)Cl₂CH₂Cl₂ (0.64 g, 0.79 mmol) and H₂O (6 mL) at rt under nitrogen atmosphere. The resulting mixture was stirred for 2 h at 90° C. under nitrogen atmosphere. On completion, the mixture was cooled to rt and concentrated under vacuum. The residue was purified by silica gel column chromatography, eluted with PE/EA (3:1), to afford the title compound (500 mg, 30% yield) as a yellow solid. LC/MS (ESI, m/z): [(M+H)]⁺=410.1, 412.1.

### Tert-butyl 2-((5-(indolin-4-yl)pyrazin-2-yl)methyl)-7-azaspiro[3.5]nonane-7-carboxylate (Intermediate TU)

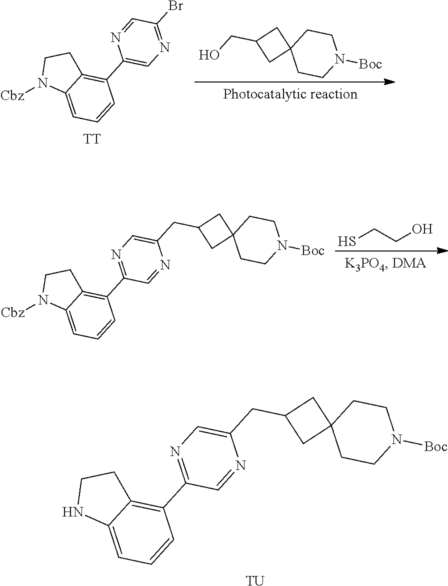

Step 1—Tert-butyl 2-((5-(1-((benzyloxy)carbonyl)indolin-4-yl)pyrazin-2-yl)methyl)-7-azaspiro[3.5]nonane-7-carboxylate. An oven-dried 50 mL vial was charged with tert-butyl 2-(hydroxymethyl)-7-azaspiro[3.5]nonane-7-carboxylate (466.81 mg, 1.829 mmol, CAS #1356476-27-1), Deoxazole (772.68 mg, 1.950 mmol) and an X-shaped magnetic stir bar. After the vial was vacuumed and refilled with nitrogen gas twice, t-BuOMe (5 mL) was added and the reaction stirred at rt for 5 min. Then, a pyridine solution (154.24 mg, 1.950 mmol in 1 mL t-BuOMe) was added dropwise at rt within 2 min. The resulting solution stirred at room temperature for 10 min. A white solid precipitated out during this time. Another oven-dried glass flask was charged with Ir[ppy]₂(dtbbpy)PF₆ (68.49 mg, 0.061 mmol), NiBr₂(dtbbpy) (29.67 mg, 0.061 mmol), quinuclidine (237.13 mg, 2.133 mmol), 2,3-dihydro-1H-isoindole-1,3-dione (35.86 mg, 0.244 mmol), benzyl 4-(5-bromopyrazin-2-yl)-2,3-dihydroindole-1-carboxylate (500 mg, 1.219 mmol, Intermediate TT), isoindoline-1,3-dione and (35.87 mg, 0.244 mmol) an X-shape magnetic stir bar. DMA (10 mL) was added to this vial under an atmosphere of nitrogen. The t-BuOMe suspension was injected through the syringe filter into the DMA solution. The resulting reaction mixture was sparged with nitrogen for 15 mins, then irradiated under 450 nm LED for 16 h. After competition of reaction, the reaction mixture was concentrated under reduced pressure. The residue was purified by reverse phase flash chromatography (Column: Spherical C18, 20-40 μm, 330 g; Mobile Phase A: Water (plus 10 mM FA); Mobile Phase B: ACN; Flow rate: 80 mL/min; Gradient: 70% B-90% B in 20 min; Detector: 254 nm; the fractions containing desired product were collected at 80% B and concentrated under reduced pressure) to afford the title compound (460 mg, 66% yield) as a yellow solid. LC/MS (ESI, m/z): [(M+H-56)]⁺=513.2.

Step 2—Tert-butyl 2-((5-(indolin-4-yl)pyrazin-2-yl)methyl)-7-azaspiro[3.5]nonane-7-carboxylate. To a stirred mixture of tert-butyl 2-((5-(1-((benzyloxy)carbonyl)indolin-4-yl)pyrazin-2-yl)methyl)-7-azaspiro[3.5]nonane-7-carboxylate (460 mg, 0.81 mmol) and K₃PO₄ (686.75 mg, 3.236 mmol) in DMA (8 mL) was added β-mercaptoethanol (126.39 mg, 1.618 mmol) at rt. The resulting mixture was stirred for 16 h at 70° C. After completion of the reaction, the reaction mixture was cooled to rt and purified directly by reverse phase flash chromatography (Column: Spherical C18, 20-40 μm, 330 g; Mobile Phase A: Water (plus 10 mM NH₄HCO₃); Mobile Phase B: ACN; Flow rate: 60 mL/min; Gradient: 60% B-80% B in 20 min; Detector: 254 nm; the fractions containing desired product were collected at 70% B and concentrated under reduced pressure) to give the title compound (220 mg, 63% yield) as a yellow solid. LC/MS (ESI, m/z): [(M+H)]⁺=435.3.

### 6-(4-(5-((7-azaspiro[3.5]nonan-2-yl)methyl)pyrazin-2-yl)indolin-1-yl)-N-((1R,2S)-2-fluorocyclopropyl)-8-(methylamino)imidazo[1,2-b]pyridazine-3-carboxamide trifluoroacetate (Intermediate TV)

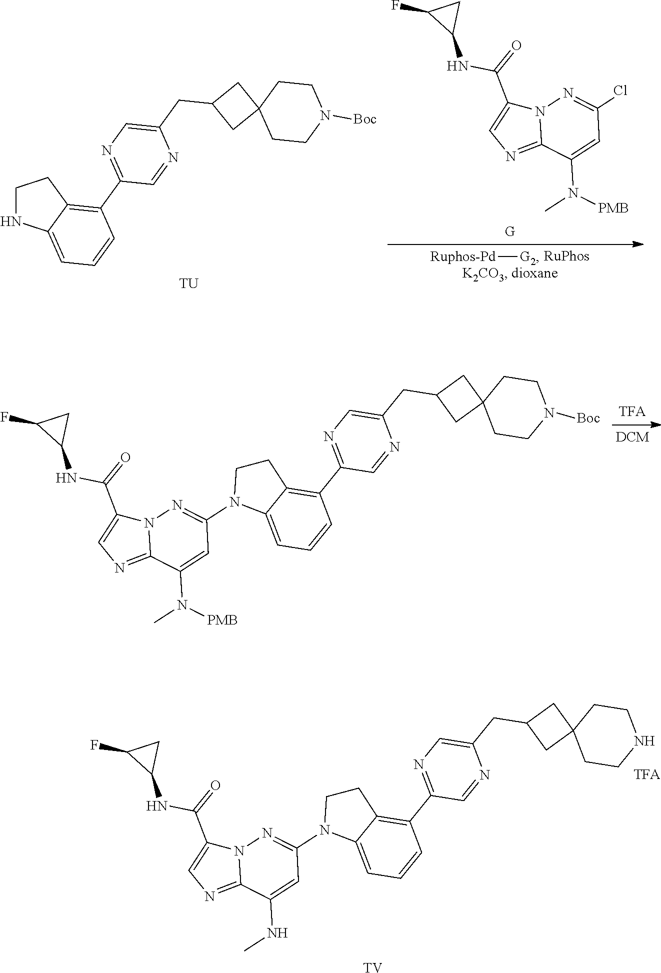

Step 1—Tert-butyl 2-((5-(1-(3-(((1R,2S)-2-fluorocyclopropyl)carbamoyl)-8-((4-methoxybenzyl)(methyl)amino)imidazo[1,2-b]pyridazin-6-yl)indolin-4-yl)pyrazin-2-yl)methyl)-7-azaspiro[3.5]nonane-7-carboxylate. To a stirred mixture of tert-butyl 2-((5-(indolin-4-yl)pyrazin-2-yl)methyl)-7-azaspiro[3.5]nonane-7-carboxylate (220 mg, 0.506 mmol, Intermediate TU) and 6-chloro-N-[(1R,2S)-2-fluorocyclopropyl]-8-{[(4-methoxyphenyl)methyl](methyl)amino}imidazo[1,2-b]pyridazine-3-carboxamide (204.44 mg, 0.506 mmol, Intermediate G) in dioxane were added K₂CO₃ (209.89 mg, 1.518 mmol), RuPhos (47.25 mg, 0.101 mmol) and RuPhos-PdCl-2nd G (47.25 mg, 0.101 mmol) at rt under nitrogen atmosphere. The resulting mixture was stirred for 2 h at 100° C. under nitrogen atmosphere. On completion, the mixture was cooled to rt and concentrated under vacuum. The residue was purified by silica gel column chromatography, eluted with DCM/EA (1:3), to afford the title compound (330 mg, 81% yield) as a yellow solid. LC/MS (ESI, m/z): [(M+H)]⁺=802.3.

Step 2—6-(4-(5-((7-Azaspiro[3.5]nonan-2-yl)methyl)pyrazin-2-yl)indolin-1-yl)-N-((1R,2S)-2-fluorocyclopropyl)-8-(methylamino)imidazo[1,2-b]pyridazine-3-carboxamide trifluoroacetate. To a stirred solution of tert-butyl 2-((5-(1-(3-(((1R,2S)-2-fluorocyclopropyl)carbamoyl)-8-((4-methoxybenzyl)(methyl)amino)imidazo[1,2-b]pyridazin-6-yl)indolin-4-yl)pyrazin-2-yl)methyl)-7-azaspiro[3.5]nonane-7-carboxylate (330 mg, 0.411 mmol) in DCM (5 mL) was added TFA (2 mL) at it and the mixture was stirred for 1 h at rt. On completion, the mixture was concentrated under vacuum. The residue was purified by trituration with diethyl ether (20 mL) to afford the title compound (300 mg) as a yellow solid. LC/MS (ESI, m/z): [(M+H)]⁺=582.3.

### 6-{4-[5-({1,1-Difluoro-3,9-diazaspiro[5.5]undecan-3-yl}methyl)-3-fluoropyridin-2-yl]-2,3-dihydroindol-1-yl}-N-[(1R,2R)-2-methoxycyclobutyl]-8-(methylamino)imidazo[1,2-b]pyridazine-3-carboxamide trifluoroacetate (Intermediate TW)

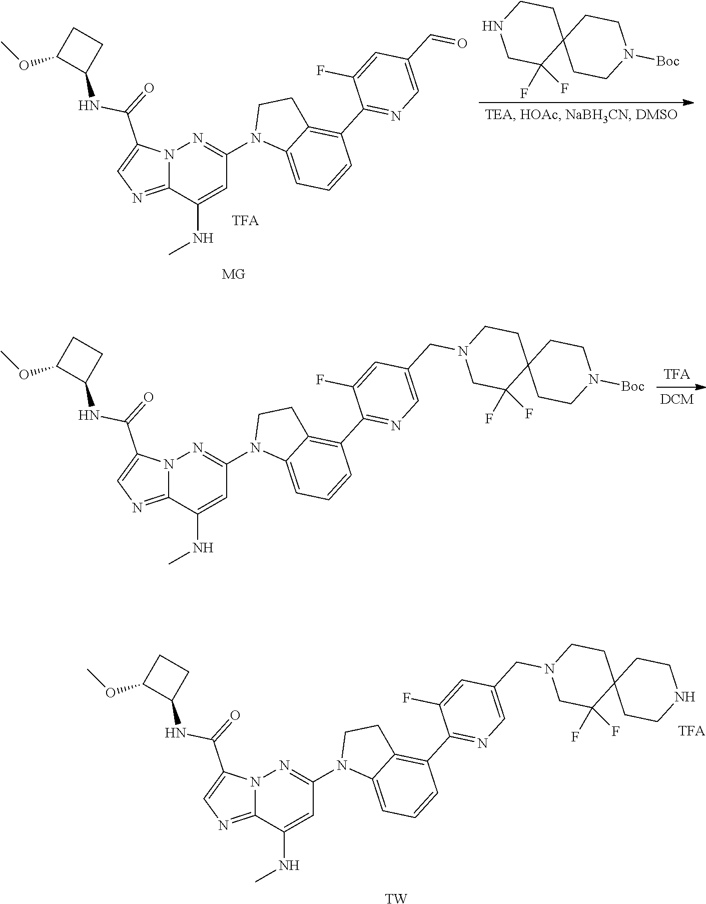

Step 1—Tert-butyl 7,7-difluoro-9-({5-fluoro-6-[1-(3-{[(1R,2R)-2-methoxycyclobutyl]carbamoyl}-8-(methylamino)imidazo[1,2-b]pyridazin-6-yl)-2,3-dihydroindol-4-yl]pyridin-3-yl}methyl)-3,9-diazaspiro[5.5]undecane-3-carboxylate. To a stirred mixture of 6-[4-(3-fluoro-5-formylpyridin-2-yl)-2,3-dihydroindol-1-yl]-N-[(1R,2R)-2-methoxycyclobutyl]-8-(methylamino)imidazo[1,2-b]pyridazine-3-carboxamide trifluoroacetate (150 mg, 0.238 mmol, Intermediate MG) in DMSO (2 mL) was added TEA (0.10 mL, 0.714 mmol) at rt under nitrogen atmosphere. To the above mixture were added tert-butyl 7,7-difluoro-3,9-diazaspiro[5.5]undecane-3-carboxylate (69.18 mg, 0.238 mmol) and HOAc (0.07 mL, 1.190 mmol) at rt. The resulting mixture was stirred for 1 h at 50° C. To the above mixture was added NaBH₃CN (29.94 mg, 0.476 mmol) at rt. The resulting mixture was stirred for an additional 2 h at 50° C. On completion, the mixture was cooled to rt and concentrated under vacuum. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 65%-85% B in 25 min; Flow rate: 80 mL/min; Detector: 220/254 nm; desired fractions were collected at 72% B and concentrated under reduced pressure) to afford the title compound (100 mg, 53% yield) as a white solid. LC/MS (ESI, m/z): [(M+H)]⁺=790.4.

Step 2—6-{4-[5-({1,1-Difluoro-3,9-diazaspiro[5.5]undecan-3-yl}methyl)-3-fluoropyridin-2-yl]-2,3-dihydroindol-1-yl}-N-[(1R,2R)-2-methoxycyclobutyl]-8-(methylamino)imidazo[1,2-b]pyridazine-3-carboxamide trifluoroacetate. To a stirred solution of tert-butyl 7,7-difluoro-9-({5-fluoro-6-[1-(3-{[(1R,2R)-2-methoxycyclobutyl]carbamoyl}-8-(methylamino)imidazo[1,2-b]pyridazin-6-yl)-2,3-dihydroindol-4-yl]pyridin-3-yl}methyl)-3,9-diazaspiro[5.5]undecane-3-carboxylate (100 mg, 0.127 mmol) in DCM (2 mL) was added TFA (1 mL) at rt under nitrogen atmosphere and the mixture was stirred for 1 h at rt. On completion, the mixture was concentrated under reduced pressure. The residue was purified by trituration with Et₂O (30 mL) to give the title compound (90 mg, 88% yield) as a yellow solid. LC/MS (ESI, m/z): [(M+H)]⁺=690.3.

### 1-(3-Chloro-5-(9-((3,3-difluoropiperidin-4-yl)methyl)-3,9-diazaspiro[5.5]undecane-3-carbonyl)-2-methylphenyl)dihydropyrimidine-2,4(1H,3H)-dionetrifluoroacetate (Intermediate TX)

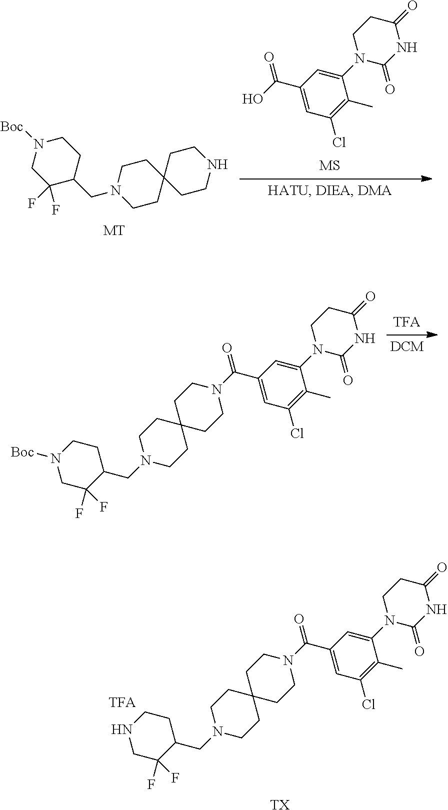

Step 1—Tert-butyl 4-((9-(3-chloro-5-(2,4-dioxotetrahydropyrimidin-1(2H)-yl)-4-methylbenzoyl)-3,9-diazaspiro[5.5]undecan-3-yl)methyl)-3,3-difluoropiperidine-1-carboxylate. To a stirred solution of tert-butyl 4-((3,9-diazaspiro[5.5]undecan-3-yl)methyl)-3,3-difluoropiperidine-1-carboxylate (230 mg, 0.594 mmol, Intermediate MT) and 3-chloro-5-(2,4-dioxotetrahydropyrimidin-1(2H)-yl)-4-methylbenzoic acid (168 mg, 0.594 mmol, Intermediate MS) in DMA (6 mL) were added DIEA (230 mg, 1.78 mmol) and HATU (339 mg, 0.891 mmol) at 0° C. under nitrogen atmosphere. The resulting mixture was stirred for 1 h at rt under nitrogen atmosphere. After completion of the reaction, the reaction mixture was purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 40%-60% B in 25 min; Flow rate: 80 mL/min; Detector: 220/254 nm; desired fractions were collected at 55% B and concentrated under reduced pressure) to afford the title compound (35 mg, 9% yield) as a white solid. LC/MS (ESI, m/z): [(M+H)]=652.4.

Step 2—1-(3-Chloro-5-(9-((3,3-difluoropiperidin-4-yl)methyl)-3,9-diazaspiro[5.5]undecane-3-carbonyl)-2-methylphenyl)dihydropyrimidine-2,4(1H,3H)-dione trifluoroacetate. To a stirred solution of tert-butyl 4-((9-(3-chloro-5-(2,4-dioxotetrahydropyrimidin-1(2H)-yl)-4-methylbenzoyl)-3,9-diazaspiro[5.5]undecan-3-yl)methyl)-3,3-difluoropiperidine-1-carboxylate (35 mg, 0.054 mmol) in DCM (3 mL) was added TFA (1 mL) at rt under air atmosphere and the mixture was stirred for 1 h at rt. On completion, the mixture was concentrated under reduced pressure. The residue was purified by trituration with Et₂O (10 mL) and dried to give the title compound (42 mg, 96% yield) as a yellow solid. LC/MS (ESI, m/z): [(M+H)]f=552.3.

### 2-Chloro-5-(2,4-dioxo-1,3-diazinan-1-yl)-4-methoxybenzoic acid (Intermediate TY)

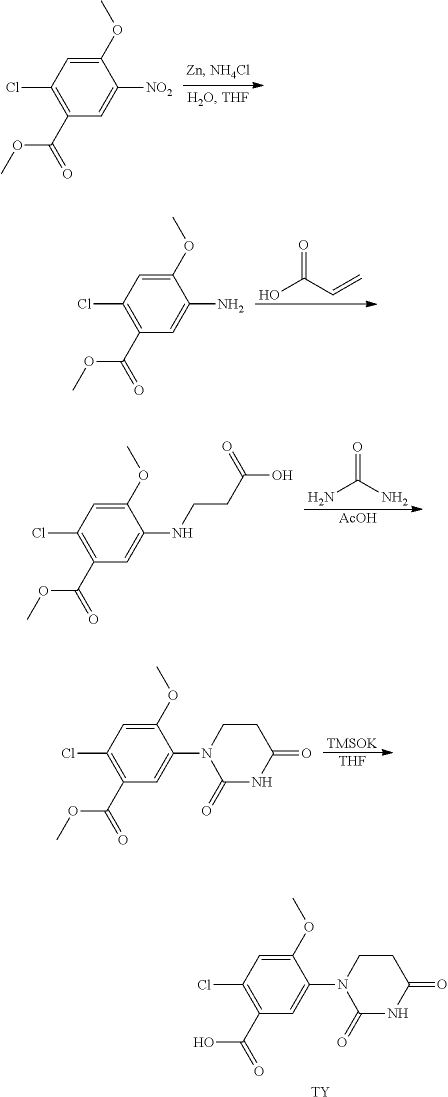

Step 1—Methyl 5-amino-2-chloro-4-methoxybenzoate. To a stirred mixture of methyl 2-chloro-4-methoxy-5-nitrobenzoate (2.00 g, 8.14 mmol) and NH4Cl (2.18 g, 40.7 mmol) in THF (30 mL) and H₂O (15 mL) was added Zn (2.66 g, 40.7 mmol) at it under nitrogen atmosphere. The resulting mixture was stirred for 2 h at rt under nitrogen atmosphere. On completion, the mixture was filtered, the filter cake was washed with acetonitrile (3×20 mL). The filtrate was concentrated under reduced pressure. The residue was purified by silica gel column chromatography, eluted with PE/EA (2:1), to afford the title compound (1.40 g, 80% yield) as a yellow solid. LC/MS (ESI, m/z): [(M+H)]⁺=216.0.

Step 2—3-{[4-Chloro-2-methoxy-5-(methoxycarbonyl)phenyl]amino}propanoic acid. To a stirred solution of methyl 5-amino-2-chloro-4-methoxybenzoate (1.37 g, 6.353 mmol) in acrylic acid (0.92 g, 12.7 mmol) at rt under nitrogen atmosphere. The resulting mixture was stirred for 1 h at 100° C. under nitrogen atmosphere. On completion, the mixture was cooled to it and diluted with water (50 mL). The resulting mixture was extracted with EtOAc (3×100 mL) and the combined organic layers were dried over anhydrous Na₂SO₄. After filtration, the filtrate was concentrated under reduced pressure to afford the title compound (1.80 g, 99% yield) as a yellow oil. LC/MS (ESI, m/z): [(M+H)]=288.1

Step 3—Methyl 2-chloro-5-(2,4-dioxo-1,3-diazinan-1-yl)-4-methoxybenzoate. To a stirred mixture of 3-{[4-chloro-2-methoxy-5-(methoxycarbonyl)phenyl]amino}propanoic acid (1.90 g, 6.60 mmol) in AcOH (20 mL) was added urea (2.78 g, 46.2 mmol) at rt under nitrogen atmosphere. The resulting mixture was stirred for 16 h at 100° C. under nitrogen atmosphere. On completion, the mixture was cooled to rt and diluted with ice water (40 mL). The precipitated solids were collected by filtration and washed with water (3×10 mL) to give the title compound (900 mg, 44% yield) as a brown yellow solid. LC/MS (ESI, m/z): [(M+H)]⁺=313.0.

Step 4—2-Chloro-5-(2,4-dioxo-1,3-diazinan-1-yl)-4-methoxybenzoic acid. To a stirred mixture of methyl 2-chloro-5-(2,4-dioxo-1,3-diazinan-1-yl)-4-methoxybenzoate (930 mg, 2.97 mmol) in THF (30 mL) was added trimethyl(potassiooxy)silane (1.14 g, 8.92 mmol) at rt under nitrogen atmosphere and the mixture was stirred for 1 h at rt under nitrogen atmosphere. On completion, the mixture was acidified to pH 3 with conc. HCl. The precipitated solids were collected by filtration and washed with water (3×10 mL) to give the title compound (600 mg, 68% yield) as a brown solid. LC/MS (ESI, m/z): [(M+H)]⁺=299.0.

### Tert-butyl (4R)-3,3-difluoro-4-[4-(4-nitrophenyl)piperazin-1-yl]piperidine-1-carboxylate (Intermediate TZ) and Tert-butyl (4S)-3,3-difluoro-4-[4-(4-nitrophenyl)piperazin-1-yl]piperidine-1-carboxylate (Intermediate UA)

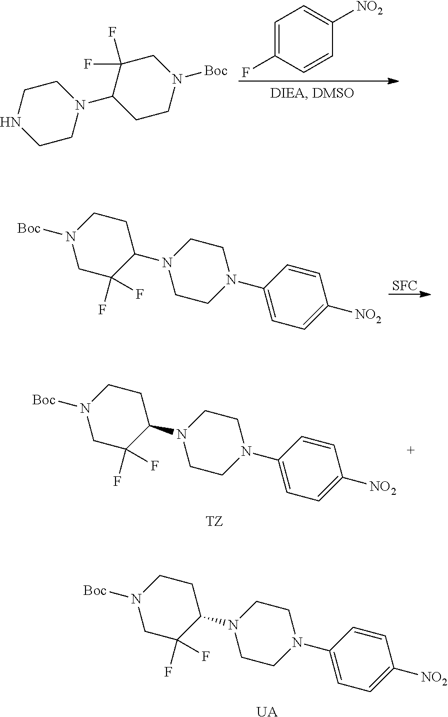

Step 1—Tert-butyl 3,3-difluoro-4-[4-(4-nitrophenyl)piperazin-1-yl]piperidine-1-carboxylate. To a stirred mixture of tert-butyl 3,3-difluoro-4-(piperazin-1-yl)piperidine-1-carboxylate (1 g, 3 mmol, CAS #2384221-09-2) and 4-fluoronitrobenzene (0.55 g, 3.93 mmol) in DMSO (20 mL) was added DIEA (1.71 mL, 9.83 mmol) at rt under nitrogen atmosphere. The resulting mixture was stirred for 4 h at 120° C. under nitrogen atmosphere. On completion, the resulting mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 60%-80% B in 25 min; Flow rate: 80 mL/min; Detector: 220/254 nm; desired fractions were collected at 76% B and concentrated under reduced pressure) to afford the title compound (1.3 g, 92% yield) as an orange solid. LC/MS (ESI, m/z): [(M+H)]⁺=427.2.

Step 2—Tert-butyl (4R)-3,3-difluoro-4-[4-(4-nitrophenyl)piperazin-1-yl]piperidine-1-carboxylate and tert-butyl (4S)-3,3-difluoro-4-[4-(4-nitrophenyl)piperazin-1-yl]piperidine-1-carboxylate. Tert-butyl 3,3-difluoro-4-[4-(4-nitrophenyl)piperazin-1-yl]piperidine-1-carboxylate (1.3 g) was separated by Prep-CHIRAL-HPLC (Column: CHIRAL ART Cellulose-SC, 2*25 cm, 5 μm; Mobile Phase A: Hex(0.5% 2M NH3-MeOH)-HPLC, Mobile Phase B: EtOH: DCM=1: 1-HPLC; Flow rate: 20 mL/min; Gradient: 30% B to 30% B in 10 min; Wave Length: 220/254 nm; RT1(min): 8.05; RT2(min): 9.68; Sample Solvent: MeOH: DCM=1: 1—HPLC; Injection Volume: 2.0 mL; Number Of Runs: 13) to afford tert-butyl (4R)-3,3-difluoro-4-[4-(4-nitrophenyl)piperazin-1-yl]piperidine-1-carboxylate (400 mg, 31% yield) as a grey solid (LC/MS (ESI, m/z): [(M+H)]⁺=427.2) and tert-butyl (4S)-3,3-difluoro-4-[4-(4-nitrophenyl)piperazin-1-yl]piperidine-1-carboxylate (450 mg, 34.62%) as a grey solid (LC/MS (ESI, m/z): [(M+H)]⁺=427.2). The absolute stereochemistry of the enantiomers was assigned arbitrarily.

### 3-[(4-{4-[(4R)-3,3-difluoropiperidin-4-yl]piperazin-1-yl}phenyl)amino]piperidine-2,6-dione (Intermediate UB)

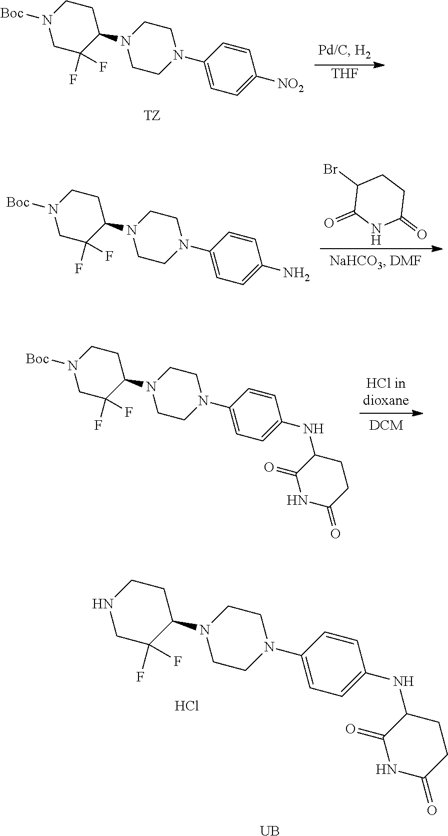

Step 1—Tert-butyl (4R)-4-[4-(4-aminophenyl)piperazin-1-yl]-3,3-difluoropiperidine-1-carboxylate. To a stirred solution of tert-butyl (4R)-3,3-difluoro-4-[4-(4-nitrophenyl)piperazin-1-yl]piperidine-1-carboxylate (400 mg, 0.938 mmol) in THF (20 mL) was added Pd/C (100 mg, 10 wt %) at rt under nitrogen atmosphere. The mixture was then hydrogenated at rt for 2 h under hydrogen atmosphere using a hydrogen balloon, filtered through a celite pad and concentrated under reduced pressure to give the title compound (300 mg, 81% yield) as a reddish brown solid. LC/MS (ESI, m/z): [(M+H)]⁺=397.2.

Step 2—Tert-butyl (4R)-4-(4-{4-[(2,6-dioxopiperidin-3-yl)amino]phenyl}piperazin-1-yl)-3,3-difluoropiperidine-1-carboxylate. To a stirred mixture of tert-butyl (4R)-4-[4-(4-aminophenyl)piperazin-1-yl]-3,3-difluoropiperidine-1-carboxylate (300 mg, 0.757 mmol) and 3-bromopiperidine-2,6-dione (581.15 mg, 3.028 mmol) in DMF (10 mL) was added NaHCO₃ (127.13 mg, 1.514 mmol) rt under nitrogen atmosphere. The resulting mixture was stirred for 16 h at 70° C. under nitrogen atmosphere. After completion of the reaction, the reaction mixture was cooled to rt and purified directly without any workup by reverse phase flash chromatography (Column: WelFlash™ C18-I, 20-40 μm, 330 g; Eluent A: Water (plus 10 mmol/L FA); Eluent B: ACN; Gradient: 15%-30% B in 20 min; Flow rate: 80 mL/min; Detector: 220/254 nm; desired fractions were collected at 23% B and concentrated under reduced pressure) to afford the title compound (300 mg, 78% yield) as a dark blue solid. LC/MS (ESI, m/z): [(M+H)]⁺=508.2.

Step 3—3-[(4-{4-[(4R)-3,3-difluoropiperidin-4-yl]piperazin-1-yl}phenyl)amino]piperidine-2,6-dione hydrochloride. To a stirred solution of tert-butyl (4R)-4-(4-{4-[(2,6-dioxopiperidin-3-yl)amino]phenyl}piperazin-1-yl)-3,3-difluoropiperidine-1-carboxylate (300 mg, 0.591 mmol) in DCM (6 mL) was added HCl (gas) in 1,4-dioxane (3 mL) at rt under nitrogen atmosphere. The resulting mixture was stirred for 1 h at rt under nitrogen atmosphere. On completion, the resulting mixture was concentrated under vacuum. The residue was purified by trituration with Et₂O (10 mL) to give the title compound (250 mg) as a brown solid. LC/MS (ESI, m/z): [(M+H)]-=408.2.

### 3-{5-[2-(3,3-Difluoropiperidin-4-yl)-2,7-diazaspiro[3.5]nonan-7-yl]-3-methyl-2-oxo-1,3-benzodiazol-1-yl}piperidine-2,6-dione 2,2,2-trifluoroacetate (Intermediate UC)

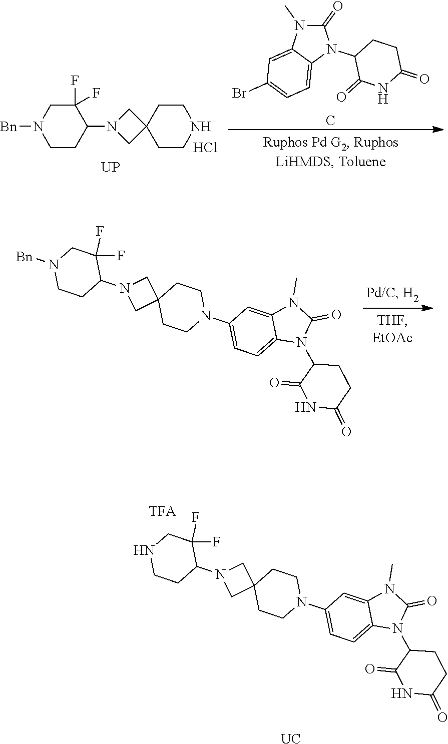

Step 1—3-{5-[2-(1-Benzyl-3,3-difluoropiperidin-4-yl)-2,7-diazaspiro[3.5]nonan-7-yl]-3-methyl-2-oxo-1,3-benzodiazol-1-yl}piperidine-2,6-dione. To a stirred solution of 2-(1-benzyl-3,3-difluoropiperidin-4-yl)-2,7-diazaspiro[3.5]nonane hydrochloride (800 mg, 2.16 mmol, Intermediate UP) and 3-(5-bromo-3-methyl-2-oxo-1,3-benzodiazol-1-yl)piperidine-2,6-dione (806.48 mg, 2.385 mmol, Intermediate C) in toluene (16 mL) were added RuPhos (222.58 mg, 0.477 mmol) and RuPhos-PdCl-2nd G (370.97 mg, 0.477 mmol) at rt under nitrogen atmosphere. To the above mixture was added LiHMDS (14.31 mL, 14.31 mmol) at rt. The resulting mixture was stirred 2 h at 80° C. On completion, mixture was cooled to rt and concentrated under reduced pressure. The residue was dissolved in H₂O (200 mL) and the mixture was extracted with EtOAc (3×200 mL). The combined organic layers were washed with brine (3×200 mL), and dried over anhydrous Na₂SO₄. After filtration, the filtrate was concentrated under reduced pressure. The crude product was purified by silica gel column chromatography (Mobile Phase A: DCM, Mobile Phase B: DCM: MeOH (10:1); Gradient: 0% B to 5% B in 25 min, 254 nm). The fractions containing the desired product were collected at 42% B; and the resulting mixture was concentrated under reduced pressure. The crude product was purified by reverse phase flash (Column: Spherical C18 Column, 20-40 μm, 330 g; Mobile Phase A: Water (0.1% NH₄HCO₃), Mobile Phase B: ACN; Flow rate: 60 mL/min; Gradient: 50% B to 80% B in 25 min, 254 nm; the fractions containing the desired product were collected at 58% B) to afford the title compound (540 mg, 38% yield) as a white solid. LC/MS (ESI, m/z): [(M+H)]⁺=593.3.

Step 2—3-{5-[2-(3,3-Difluoropiperidin-4-yl)-2,7-diazaspiro[3.5]nonan-7-yl]-3-methyl-2-oxo-1,3-benzodiazol-1-yl}piperidine-2,6-dione trifluoroacetate. To a stirred solution of 3-{5-[2-(1-benzyl-3,3-difluoropiperidin-4-yl)-2,7-diazaspiro[3.5]nonan-7-yl]-3-methyl-2-oxo-1,3-benzodiazol-1-yl}piperidine-2,6-dione (200 mg, 0.337 mmol) in THE (10 mL) and EA (20 mL) was added Pd/C (200 mg, 1.88 mmol) at rt under nitrogen atmosphere. The resulting mixture was stirred overnight at 50° C. under hydrogen atmosphere. On completion, the mixture was cooled to rt and filtered. The filter cake was washed with DCM (5×30 mL) and the filtrate was concentrated under reduced pressure. The crude product was purified by reverse phase flash (Column: Spherical C18 Column, 20-40 μm, 330 g; Mobile Phase A: Water (0.1% TFA), Mobile Phase B: ACN; Flow rate: 80 mL/min; Gradient: 35% B to 60% B in 25 min, 254 nm; the fractions containing the desired product were collected at 48% B) to afford the title compound (50 mg, 30% yield) as a white solid. LC/MS (ESI, m/z): [(M+H)]=503.2.

### N-cyclopropyl-6-(4-(5-((1,1-difluoro-3,9-diazaspiro[5.5]undecan-3-yl)methyl)pyridin-2-yl)indolin-1-yl)-8-(methylamino)imidazo[1,2-b]pyridazine-3-carboxamide (Intermediate UD)

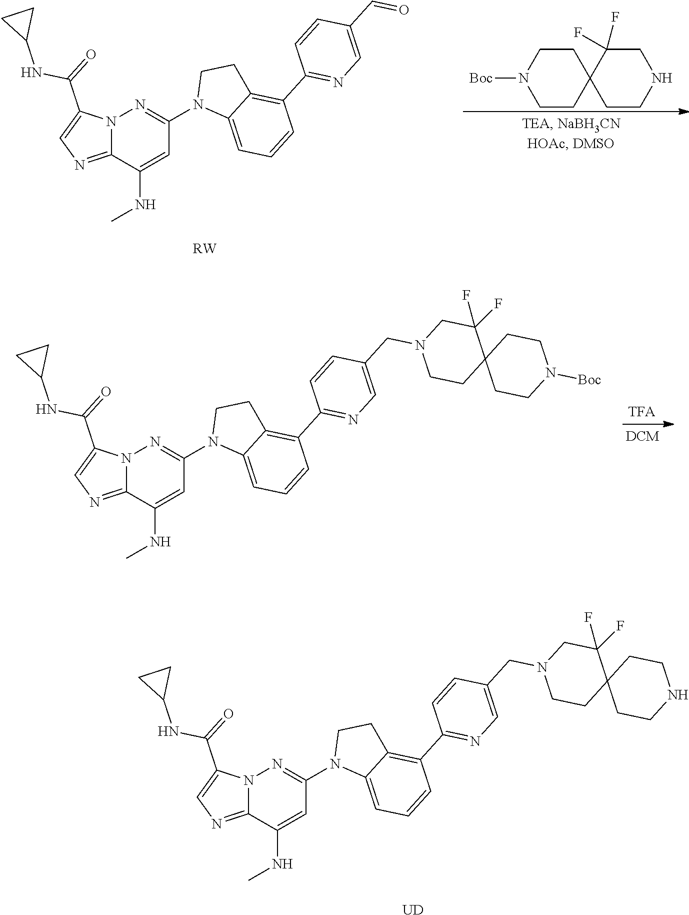 [SMILES: CNc1cc(N2CCc3c(-c4ccc(C=O)cn4)cccc32)nn2c(C(=O)NC3CC3)cnc12 ; CC(C)(C)OC(=O)N1CCC2(CCNCC2(F)F)CC1 ; "RW" ; CNc1cc(N2CCc3c(-c4ccc(CN5CCC6(CCN(C(=O)OC(C)(C)C)CC6)C(F)(F)C5)cn4)cccc32)nn2c(C(=O)NC3CC3)cnc12 ; CNc1cc(N2CCc3c(-c4ccc(CN5CCC6(CCNCC6)C(F)(F)C5)cn4)cccc32)nn2c(C(=O)NC3CC3)cnc12 ; "UD"]

Step 1—Tert-butyl 9-((6-(1-(3-(cyclopropylcarbamoyl)-8-(methylamino)imidazo[1,2-b]pyridazin-6-yl)indolin-4-yl)pyridin-3-yl)methyl)-7,7-difluoro-3,9-diazaspiro[5.5]undecane-3-carboxylate. To a stirred mixture of tert-butyl 7,7-difluoro-3,9-diazaspiro[5.5]undecane-3-carboxylate (200 mg, 0.689 mmol) in DMSO (3 mL) was added Et₃N (0.04 mL) followed by the addition of N-cyclopropyl-6-[4-(5-formylpyridin-2-yl)-2,3-dihydroindol-1-yl]-8-(methylamino)imidazo[1,2-b]pyridazine-3-carboxamide (312.38 mg, 0.689 mmol, Intermediate RW) at rt. To the above mixture was added HOAc (0.06 mL) and the resulting mixture was stirred for 1 h at 50° C. The mixture was then allowed to cool down to rt. To the above mixture was added NaBH₃CN (216.42 mg, 3.445 mmol) in portions. The resulting mixture was stirred for additional 2 h at 50° C. On completion, the mixture was cooled to rt and purified by reverse phase flash chromatography (Column: Spherical C18, 20-40 μm, 330 g; Mobile Phase A: Water (plus 10 mM NH₄HCO₃/FA); Mobile Phase B: ACN; Flow rate: 80 mL/min; Gradient: 50% B-70% B in 20 min; Detector: 254 nm; the fractions containing desired product were collected at 60% B and concentrated under reduced pressure) to afford the title compound (300 mg, 60% yield) as a yellow solid. LC/MS (ESI, m/z): [(M+H)]⁺=728.5.

Step 2—N-cyclopropyl-6-(4-(5-((1,1-difluoro-3,9-diazaspiro[5.5]undecan-3-yl)methyl)pyridin-2-yl)indolin-1-yl)-8-(methylamino)imidazo[1,2-b]pyridazine-3-carboxamide trifluoroacetate. To a stirred solution of tert-butyl 9-[(6-{1-[3-(cyclopropylcarbamoyl)-8-(methylamino)imidazo[1,2-b]pyridazin-6-yl]-2,3-dihydroindol-4-yl}pyridin-3-yl)methyl]-7,7-difluoro-3,9-diazaspiro[5.5]undecane-3-carboxylate (300 mg, 0.412 mmol) in DCM (9 mL) was added TFA (3 mL). The resulting mixture was stirred for 1 h at rt. On completion, the mixture was concentrated under vacuum. The residue was purified by trituration with diethyl ether (10 mL) to afford the title compound (290 mg, 97% yield) as a yellow solid. LC/MS (ESI, m/z): [(M+H)]⁺=628.4.

### 1′-Benzyl-3′,3′-difluoro-4-(piperazin-1-yl)-1,4′-bipiperidine (Intermediate UE)

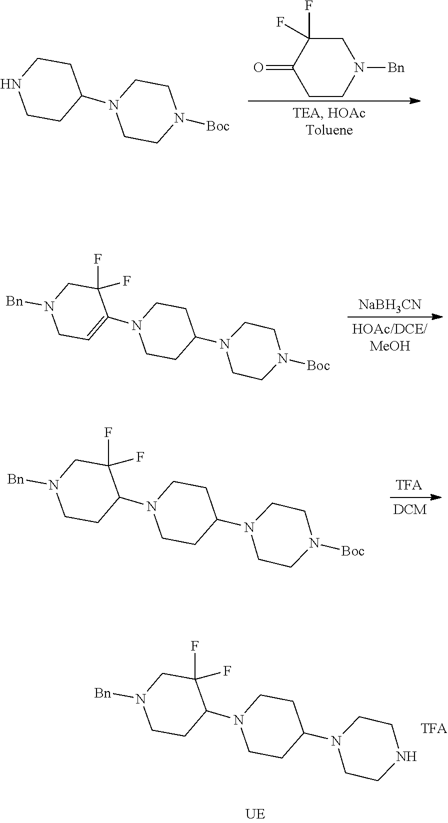 [SMILES: CC(C)(C)OC(=O)N1CCN(C2CCNCC2)CC1 ; O=C1CCN(Cc2ccccc2)CC1(F)F ; CC(C)(C)OC(=O)N1CCN(C2CCN(C3=CCN(Cc4ccccc4)CC3(F)F)CC2)CC1 ; "NaBH3CN" ; CC(C)(C)OC(=O)N1CCN(C2CCN(C3CCN(Cc4ccccc4)CC3(F)F)CC2)CC1 ; FC1(F)CN(Cc2ccccc2)CCC1N1CCC(N2CCNCC2)CC1]

Step 1—Tert-butyl 4-(1-(1-benzyl-3,3-difluoro-1,2,3,6-tetrahydropyridin-4-yl)piperidin-4-yl)piperazine-1-carboxylate. Tert-butyl 4-(piperidin-4-yl)piperazine-1-carboxylate (CAS #205059-24-1) (4.50 g, 16.70 mmol) was dissolved in toluene (50 mL). To the solution were added TEA (3.78 mL, 27.22 mmol), 1-benzyl-3,3-difluoropiperidin-4-one (CAS #1039741-54-2) (5.64 g, 25.05 mmol) and HOAc (2.88 mL, 50.279 mmol). The reaction mixture was then heated to 110° C. and reflux with a water separator for 3 h. On completion, the mixture was concentrated under reduced pressure. The resulting mixture was diluted with water (400 mL). The resulting mixture was extracted with EtOAc (2×200 mL). The combined organic layers were washed with brine (300 mL), and dried over anhydrous Na₂SO₄. After filtration, the filtrate was concentrated under reduced pressure to afford to give the title compound (7.40 g, 92% yield) as a white solid. LC/MS (ESI, m/z): [(M+1)]⁺=477.3.

Step 2—Tert-butyl 4-(1′-benzyl-3′,3′-difluoro-[1,4′-bipiperidin]-4-yl)piperazine-1-carboxylate. To a stirred mixture of tert-butyl 4-(1-(1-benzyl-3,3-difluoro-1,2,3,6-tetrahydropyridin-4-yl)piperidin-4-yl)piperazine-1-carboxylate (7.40 g, 15.5 mmol) in DCE (70 mL) and MeOH (70 mL) was added NaBH₃CN (4.88 g, 77.6 mmol) in portions at rt under nitrogen atmosphere. To the above mixture was added HOAc (7 mL) dropwise over 3 min at rt. The resulting mixture was stirred for additional 16 h at rt. On completion, the mixture was concentrated under reduced pressure. The resulting mixture was diluted with water (800 mL) and extracted with EtOAc (2×400 mL). The combined organic layers were washed with brine (400 mL), and dried over anhydrous Na₂SO₄. After filtration, the filtrate was concentrated under reduced pressure. The residue was purified by silica gel column chromatography, eluted with CH₂Cl₂/MeOH (20:1) to afford the title compound (7.4 g, 99% yield) as a light yellow solid. LC/MS (ESI, m/z): [(M+1)]⁺=479.3.

Step 3—1′-Benzyl-3′,3′-difluoro-4-(piperazin-1-yl)-1,4′-bipiperidine trifluoroacetate. To a solution of tert-butyl 4-{1′-benzyl-3′,3′-difluoro-[1,4′-bipiperidin]-4-yl}piperazine-1-carboxylate (7.4 g, 15.46 mmol) in DCM (80 mL) was added TFA (40 mL) dropwise at rt. The reaction mixture was stirred for 2 h at rt under nitrogen atmosphere. On completion, the mixture was concentrated under reduced pressure. The residue was triturated with Et₂O to afford the title compound (7.3 g, 96% yield) as an off-white solid. LC/MS (ESI, m/z): [(M+1)]⁺=379.2.

### 3-(5-(4-(3′,3′-Difluoro-[1,4′-bipiperidin]-4-yl)piperazin-1-yl)-3-methyl-2-oxo-2,3-dihydro-1H-benzo[d]imidazol-1-yl)piperidine-2,6-dione (Intermediate UF)

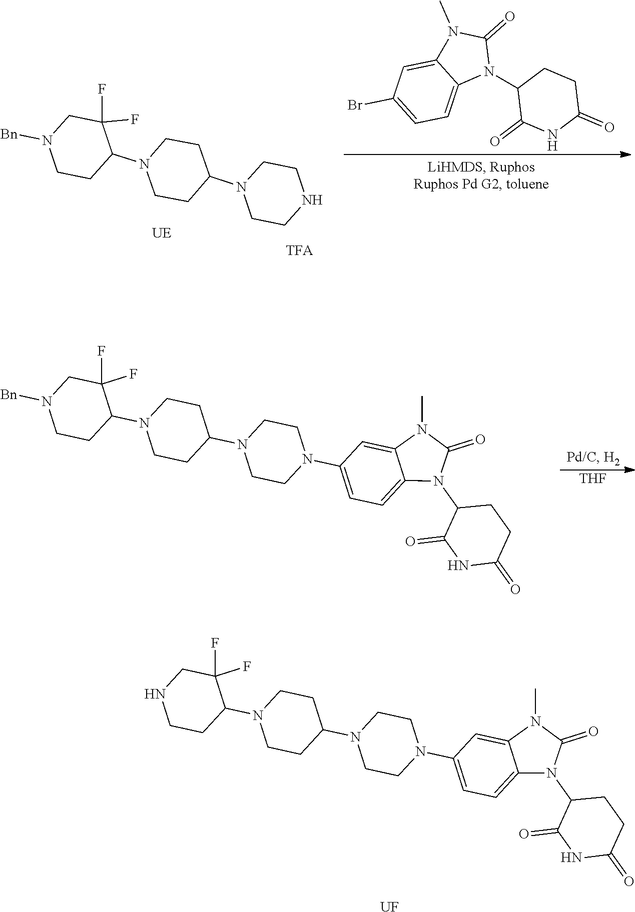

Step 1—3-(5-(4-(1′-Benzyl-3′,3′-difluoro-[1,4′-bipiperidin]-4-yl)piperazin-1-yl)-3-methyl-2-oxo-2,3-dihydro-1H-benzo[d]imidazol-1-yl)piperidine-2,6-dione. To a stirred solution of 1′-benzyl-3′,3′-difluoro-4-(piperazin-1-yl)-1,4′-bipiperidine trifluoroacetate (300 mg, 0.630 mmol, Intermediate UE) and 3-(5-bromo-3-methyl-2-oxo-1,3-benzodiazol-1-yl)piperidine-2,6-dione (CAS #2300099-98-1) (255 mg, 0.756 mmol, in toluene (6 mL) was added RuPhos (58 mg, 0.126 mmol) and RuPhos-PdCl-2nd G (97 mg, 0.126 mmol) at rt under nitrogen atmosphere. To the above mixture was added LiHMDS (632 mg, 3.780 mmol) dropwise over 5 min at 0° C. The resulting mixture was stirred for an additional 2 h at 80° C. On completion, the mixture was cooled to rt and concentrated under reduced pressure. The residue was purified by silica gel column chromatography, eluted with CH₂Cl₂/MeOH (10:1) to afford the title compound (150 mg, 37% yield) as a light brown solid. LC/MS (ESI, m/z): [(M+1)]⁺=636.4.

Step 2—3-(5-(4-(3′,3′-Difluoro-[1,4′-bipiperidin]-4-yl)piperazin-1-yl)-3-methyl-2-oxo-2,3-dihydro-TH-benzo[d]imidazol-1-yl)piperidine-2,6-dione. To a solution of 3-[5-(4-{1′-benzyl-3′,3′-difluoro-[1,4′-bipiperidin]-4-yl}piperazin-1-yl)-3-methyl-2-oxo-1,3-benzodiazol-1-yl]piperidine-2,6-dione (150 mg, 0.236 mmol) in THF (5 mL) was added Pd/C (125.54 mg, 1.180 mmol) under nitrogen atmosphere. The reaction system was degassed under vacuum and purged with H₂ several times, then it was hydrogenated under H₂ balloon (˜1 atm) at 25° C. for 2 h. After completion of the reaction, Pd/C was filtered off through celite and the filter cake was washed with MeOH (3×30 mL). The corresponding filtrate was concentrated under reduced pressure to afford the title compound (75 mg, 58% yield) as a white solid. LC/MS (ESI, m/z): [(M+1)]⁺=546.3.

### 6-{4-[5-({5,5-Difluoro-2,7-diazaspiro[3.5]nonan-2-yl}methyl)pyridin-2-yl]-2,3-dihydroindol-1-yl}-N-[(1R,2S)-2-fluorocyclopropyl]-8-(methylamino)imidazo[1,2-b]pyridazine-3-carboxamide trifluoroacetate (Intermediate UG)

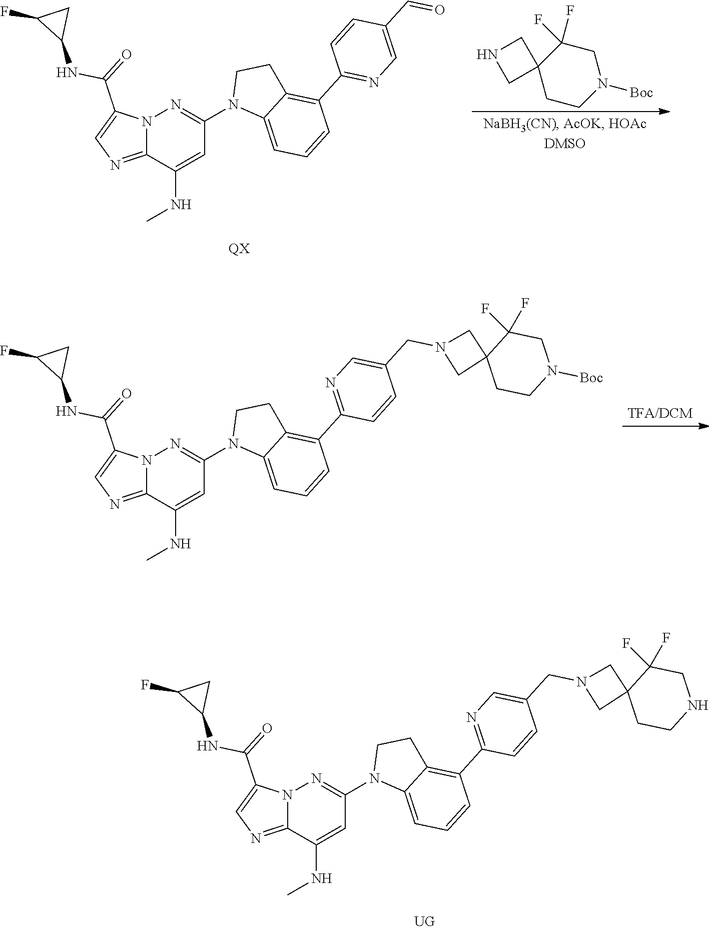

Step 1—Tert-butyl 5,5-difluoro-2-({6-[1-(3-{[(1R,2S)-2-fluorocyclopropyl]carbamoyl}-8-(methylamino)imidazo[1,2-b]pyridazin-6-yl)-2,3-dihydroindol-4-yl]pyridin-3-yl}methyl)-2,7-diazaspiro[3.5]nonane-7-carboxylate. To a stirred mixture of N-[(1R,2S)-2-fluorocyclopropyl]-6-[4-(5-formylpyridin-2-yl)-2,3-dihydroindol-1-yl]-8-(methylamino)imidazo[1,2-b]pyridazine-3-carboxamide (200 mg, 0.42 mmol, Intermediate QX) and tert-butyl 5,5-difluoro-2,7-diazaspiro[3.5]nonane-7-carboxylate (122.39 mg, 0.466 mmol) in DMSO (2 mL) was added AcOK (208 mg, 2.12 mmol) at rt. To the above mixture was added AcOH (127 mg, 2.12 mmol) at rt. The resulting mixture was stirred for an additional 0.5 h at 50° C. To the above mixture was added NaBH₃CN (133 mg, 2.12 mmol) at rt. The resulting mixture was stirred for an additional 2 h at 50° C. On completion, the reaction was cooled to rt and quenched by the addition of water (50 mL) at rt. The resulting mixture was extracted with EtOAc (3×60 mL). The combined organic layers were washed with brine (2×40 mL), and dried over anhydrous Na₂SO₄. After filtration, the filtrate was concentrated under reduced pressure. The residue was purified by silica gel column chromatography, eluted with CH₂Cl₂/MeOH (10/1) to afford the title compound (178 mg, 59% yield) as an off-white solid. LC/MS (ESI, m/z): [(M+H)]⁺=718.4.

Step 2—6-{4-[5-({5,5-Difluoro-2,7-diazaspiro[3.5]nonan-2-yl}methyl)pyridin-2-yl]-2,3-dihydroindol-1-yl}-N-[(1R,2S)-2-fluorocyclopropyl]-8-(methylamino)imidazo[1,2-b]pyridazine-3-carboxamide trifluoroacetate. To a stirred solution of tert-butyl 5,5-difluoro-2-({6-[1-(3-{[(1R,2S)-2-fluorocyclopropyl]carbamoyl}-8-(methylamino)imidazo[1,2-b]pyridazin-6-yl)-2,3-dihydroindol-4-yl]pyridin-3-yl}methyl)-2,7-diazaspiro[3.5]nonane-7-carboxylate (178 mg, 0.25 mmol) in DCM (4 mL) was added TFA (2 mL) dropwise at rt. The resulting mixture was stirred for 2 h at rt. On completion, the mixture was concentrated under reduced pressure. The residue was purified by trituration with Et₂O (5 mL) to afford the title compound (196 mg) as a brown yellow solid. LC/MS (ESI, m/z): [(M+H)]⁺=618.3.

### 3-(5-Bromo-4-fluoro-3-methyl-2-oxo-2,3-dihydro-LH-benzo[d]imidazol-1-yl)-1-(4-methoxybenzyl)piperidine-2,6-dione (Intermediate UH)

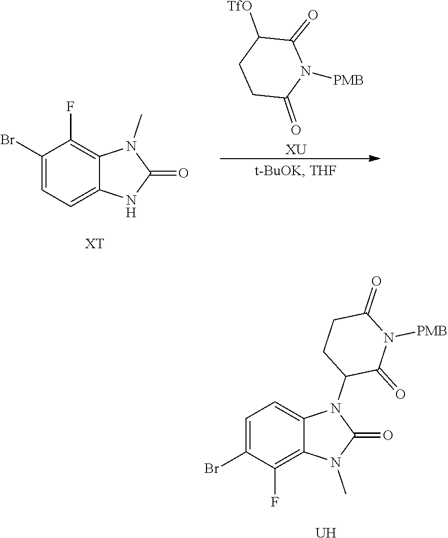

Step 1—3-(5-Bromo-4-fluoro-3-methyl-2-oxo-2,3-dihydro-1H-benzo[d]imidazol-1-yl)-1-(4-methoxybenzyl)piperidine-2,6-dione. To a stirred solution of 6-bromo-7-fluoro-1-methyl-3H-1,3-benzodiazol-2-one (3 g, 12 mmol, Intermediate XT) in THF (40 mL) was added t-BuOK (1.65 g, 14.7 mmol) in portions at 0° C. The resulting mixture was stirred for 30 min at 0° C. To the above mixture was added a solution of 1-[(4-methoxyphenyl)methyl]-2,6-dioxopiperidin-3-yl trifluoromethanesulfonate (5.60 g, 14.7 mmol, Intermediate XU) in THF (5 mL) dropwise at 0° C. The resulting mixture was stirred for 2 h at rt. On completion, the mixture was diluted with water (90 mL) and extracted with EtOAc (3×40 mL). The combined organic layers were washed with brine (3×120 mL), and dried over anhydrous Na₂SO₄. After filtration, the filtrate was concentrated under reduced pressure. The crude product was purified by reverse phase flash chromatography (WelFlash™ C18-I, 20-40 μm, 330 g; Eluent A: Water (10 mmol/L NH₄HCO₃); Eluent B: ACN; Gradient: 30%-65% B in 30 min; Flow rate: 80 mL/min; Detector: 220 nm; desired fractions were collected at 58% B and concentrated under reduced pressure) to afford the title compound (3.7 g, 60% yield) as a grey solid. LC/MS (ESI, m/z): [(M+1)]=476.1.

### 3-(4-Fluoro-3-methyl-2-oxo-5-(piperazin-1-yl)-2,3-dihydro-1H-benzo[d]imidazol-1-yl)piperidine-2,6-dione (Intermediate UI)

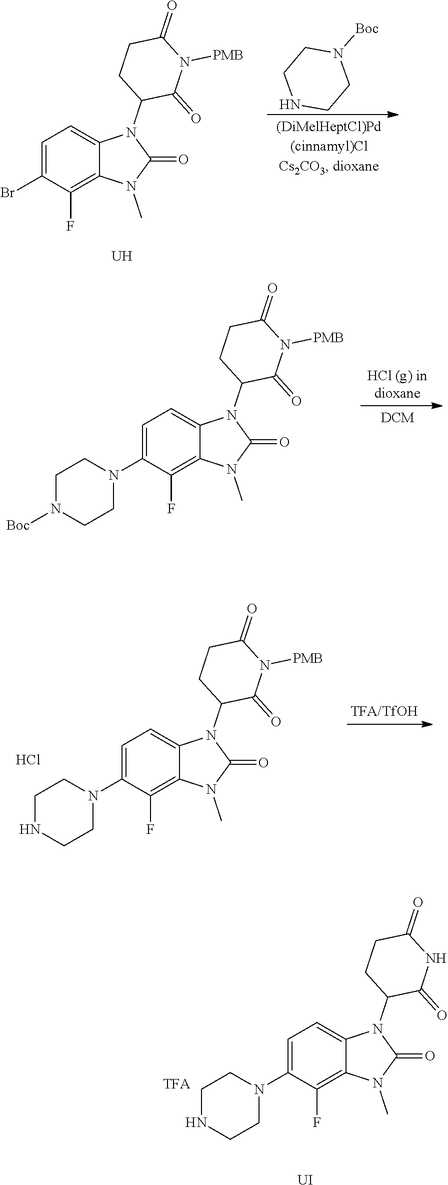

Step 1—Tert-butyl 4-(4-fluoro-1-(1-(4-methoxybenzyl)-2,6-dioxopiperidin-3-yl)-3-methyl-2-oxo-2,3-dihydro-TH-benzo[d]imidazol-5-yl)piperazine-1-carboxylate. To a stirred solution of 3-(5-bromo-4-fluoro-3-methyl-2-oxo-2,3-dihydro-1H-benzo[d]imidazol-1-yl)-1-(4-methoxybenzyl)piperidine-2,6-dione (500 mg, 1.05 mmol, Intermediate UH) and tert-butyl piperazine-1-carboxylate (293 mg, 1.57 mmol) in 1,4-dioxane (10.00 mL) were added Cs₂CO₃ (1.03 g, 3.15 mmol) and (DiMelHeptCl)Pd(cinnamyl)Cl (122 mg, 0.105 mmol) at rt under nitrogen atmosphere. The resulting mixture was stirred for overnight at 90° C. under nitrogen atmosphere. On completion, the mixture was cooled to rt and concentrated under reduced pressure. The residue was dissolved in DMSO (5 mL). The residue product was purified by reverse phase flash chromatography (WelFlash™ C18-I, 20-40 μm, 330 g; Eluent A: Water (plus or 10 mmol/L NH₄HCO₃); Eluent B: ACN; Gradient: 40%-70% B in 25 min; Flow rate: 80 mL/min; Detector: 220/254 nm; desired fractions were collected at 68% B and concentrated under reduced pressure) to afford the title compound (380 mg, 53% yield) as a light brown solid. LC/MS (ESI, m/z): [(M+1)]⁺=582.3.

Step 2—3-(4-Fluoro-3-methyl-2-oxo-5-(piperazin-1-yl)-2,3-dihydro-1H-benzo[d]imidazol-1-yl)-1-(4-methoxybenzyl)piperidine-2,6-dione hydrochloride. To a stirred solution of tert-butyl 4-(4-fluoro-1-(1-(4-methoxybenzyl)-2,6-dioxopiperidin-3-yl)-3-methyl-2-oxo-2,3-dihydro-1H-benzo[d]imidazol-5-yl)piperazine-1-carboxylate (380 mg, 0.653 mmol) in DCM (1 mL) was added HCl (gas) in 1,4-dioxane (1.00 mL) at rt under air atmosphere. The resulting mixture was stirred for 1 h at rt. On completion, the resulting mixture was concentrated under reduced pressure. The crude was triturated with Et₂O to afford the title compound (300 mg, 75% yield) as a yellow solid. LC/MS (ESI, m/z): [(M+1)]=482.2.

Step 3—3-(4-Fluoro-3-methyl-2-oxo-5-(piperazin-1-yl)-2,3-dihydro-TH-benzo[d]imidazol-1-yl)piperidine-2,6-dione trifluoroacetate. To a stirred solution of 3-(4-fluoro-3-methyl-2-oxo-5-(piperazin-1-yl)-2,3-dihydro-1H-benzo[d]imidazol-1-yl)-1-(4-methoxybenzyl)piperidine-2,6-dione hydrochloride (100 mg, 0.209 mmol) in TFA (2 mL) was added trifluoromethanesulfonic acid (100 uL) at rt. The resulting mixture was stirred for 1 h at 70° C. On completion, the mixture was cooled to rt and concentrated under reduced pressure. The crude was triturated with Et₂O to afford the title compound (80 mg, TFA salt). LC/MS (ESI, m/z): [(M+1)]⁺=362.2.

### Tert-butyl 2-{[6-(2,3-dihydro-LH-indol-4-yl)pyridin-3-yl]oxy}-7-azaspiro[3.5]nonane-7-carboxylate (Intermediate UJ)

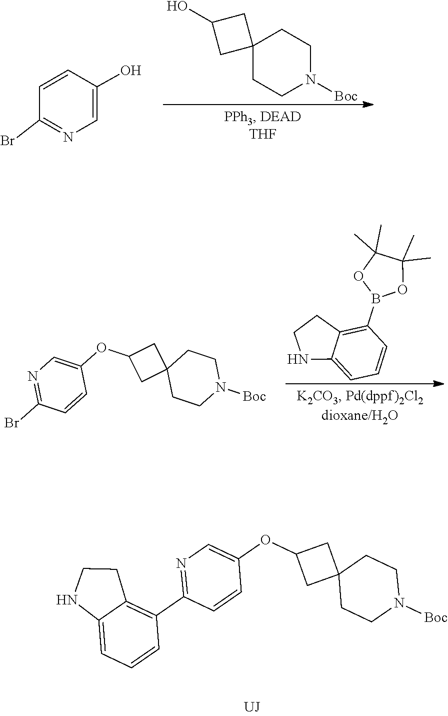 [SMILES: Oc1ccc(Br)nc1 ; CC(C)(C)OC(=O)N1CCC2(CC1)CC(O)C2 ; CC(C)(C)OC(=O)N1CCC2(CC1)CC(Oc1ccc(Br)nc1)C2 ; CC1(C)OB(c2cccc3c2CCN3)OC1(C)C ; CC(C)(C)OC(=O)N1CCC2(CC1)CC(Oc1ccc(-c3cccc4c3CCN4)nc1)C2]

Step 1—Tert-butyl 2-[(6-bromopyridin-3-yl)oxy]-7-azaspiro[3.5]nonane-7-carboxylate. To a stirred mixture of tert-butyl 2-hydroxy-7-azaspiro[3.5]nonane-7-carboxylate (6.93 g, 28.7 mmol), PPh₃ (11.31 g, 43.10 mmol) and 6-bromopyridin-3-ol (5 g, 30 mmol) in THF (100 mL) was added DEAD (7.51 g, 43.104 mmol) at rt. The resulting mixture was stirred for 4 h at rt. On completion, the reaction was quenched by the addition of water (300 mL) at rt. The resulting mixture was extracted with EtOAc (3×400 mL). The combined organic layers were washed with brine (2×300 mL), and dried over anhydrous Na₂SO₄. After filtration, the filtrate was concentrated under reduced pressure. The residue was purified by silica gel column chromatography, eluted with PE/EA (8:1˜1:2), to afford the title compound (9.81 g, 86% yield) as a pink solid. LC/MS (ESI, m/z): [(M+H)]=397.0, 399.0.

Step 2—Tert-butyl 2-{[6-(2,3-dihydro-1H-indol-4-yl)pyridin-3-yl]oxy}-7-azaspiro[3.5]nonane-7-carboxylate. To a stirred mixture of tert-butyl 2-[(6-bromopyridin-3-yl)oxy]-7-azaspiro[3.5]nonane-7-carboxylate (1 g, 3 mmol) and 4-(4,4,5,5-tetramethyl-1,3,2-dioxaborolan-2-yl)-2,3-dihydro-1H-indole (0.62 g, 2.5 mmol) in dioxane (8 mL) and H₂O (2 mL) were added K₂CO₃ (1.04 g, 7.55 mmol) and Pd(dppf)Cl₂·CH₂Cl₂ (0.21 g, 0.25 mmol) at rt. The reaction mixture was degassed with nitrogen three times and stirred for 2 h at 90° C. On completion, the reaction was cooled to rt and quenched with water (100 mL) at rt. The resulting mixture was extracted with EtOAc (3×200 mL). The combined organic layers were washed with brine (2×200 mL) and dried over anhydrous Na₂SO₄. After filtration, the filtrate was concentrated under reduced pressure. The residue was purified by reversed-phase flash chromatography (column, C18 silica gel; mobile phase, MeCN in Water (10 mmol/L NH₄HCO₃), 20% to 95% gradient in 25 min; detector, UV 254 nm; the fractions containing the desired product were combined and concentrated) to afford the title compound (490 mg, 45% yield) as a brown yellow oil. LC/MS (ESI, m/z): [(M+H)]⁺=436.3.

### 6-[4-(5-{7-Azaspiro[3.5]nonan-2-yloxy}pyridin-2-yl)-2,3-dihydroindol-1-yl]-N-[(1R,2S)-2-fluorocyclopropyl]-8-(methylamino)imidazo[1,2-b]pyridazine-3-carboxamide (Intermediate UK)

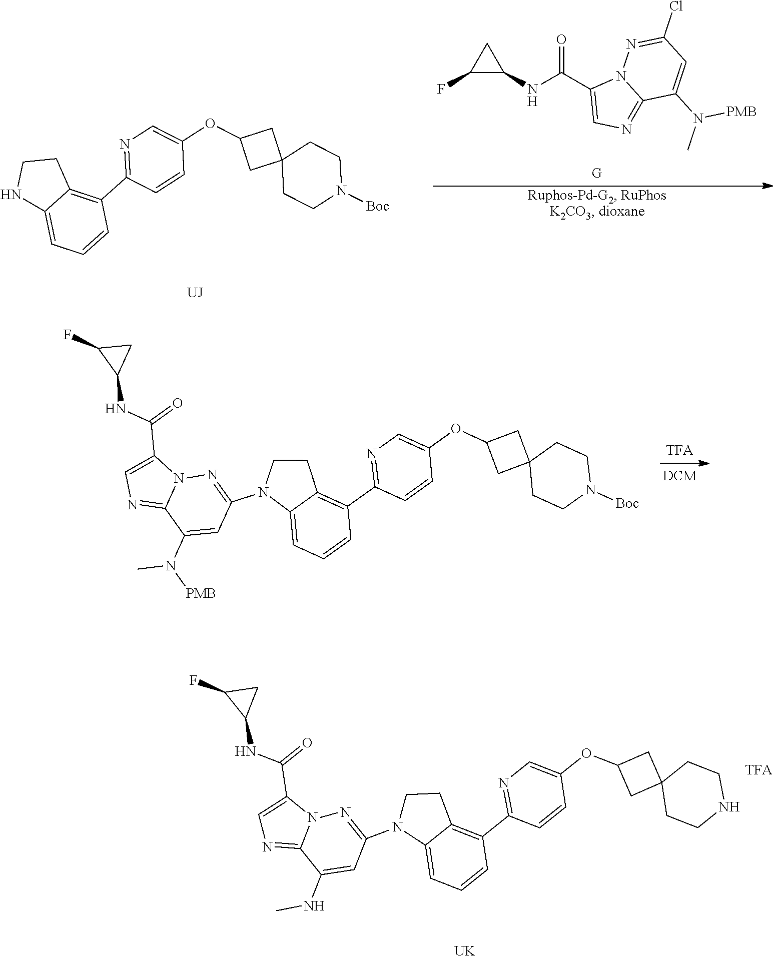

Step 1—Tert-butyl 2-({6-[l-(3-{[(1R,2S)-2-fluorocyclopropyl]carbamoyl}-8-{1[(4-methoxyphenyl)methyl](methyl)amino}imidazo[1,2-b]pyridazin-6-yl)-2,3-dihydroindol-4-yl]pyridin-3-yl}oxy)-7-azaspiro[3.5]nonane-7-carboxylate. To a stirred mixture of tert-butyl 2-{[6-(2,3-dihydro-1H-indol-4-yl)pyridin-3-yl]oxy}-7-azaspiro[3.5]nonane-7-carboxylate (460 mg, 1.06 mmol, Intermediate UJ), K₂CO₃ (437.87 mg, 3.168 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (426.49 mg, 1.056 mmol, Intermediate G) in dioxane (10 mL) were added RuPhos-PdCl-2nd G (164.27 mg, 0.211 mmol) and RuPhos (98.56 mg, 0.211 mmol) at rt. The reaction mixture was degassed with nitrogen for three times and stirred for 2 h at 100° C. On completion, the reaction was cooled to rt and quenched by the addition of water (100 mL) at rt. The resulting mixture was extracted with EtOAc (3×100 mL). The combined organic layers were washed with brine (2×100 mL), and dried over anhydrous Na₂SO₄. After filtration, the filtrate was concentrated under reduced pressure. The residue was purified by silica gel column chromatography, eluted with DCM/EA (1:1), to afford the title compound (177 mg, 21%) as a brown yellow solid. LC/MS (ESI, m/z): [(M+H)]f=803.4.

Step 2—6-[4-(5-{7-Azaspiro[3.5]nonan-2-yloxy}pyridin-2-yl)-2,3-dihydroindol-1-yl]-N-[(1R,2S)-2-fluorocyclopropyl]-8-(methylamino)imidazo[1,2-b]pyridazine-3-carboxamide trifluoroacetate. To a stirred mixture of tert-butyl 2-({6-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyridin-3-yl}oxy)-7-azaspiro[3.5]nonane-7-carboxylate (177 mg, 0.220 mmol) in DCM (2 mL) was added TFA (2 mL) dropwise at rt. The resulting mixture was stirred for 1 h at rt. On completion, the mixture was concentrated under reduced pressure. The residue was purified by trituration with Et₂O (10 mL) to afford the title compound (196 mg) as a brown yellow solid. LC/MS (ESI, m/z): [(M+H)]f=583.4.

### 3-(5-(4-((R)-3,3-difluoropiperidin-4-yl)piperazin-1-yl)-3-methyl-2-oxo-2,3-dihydro-1H-benzo[d]imidazol-1-yl)piperidine-2,6-dione (Intermediate UL)

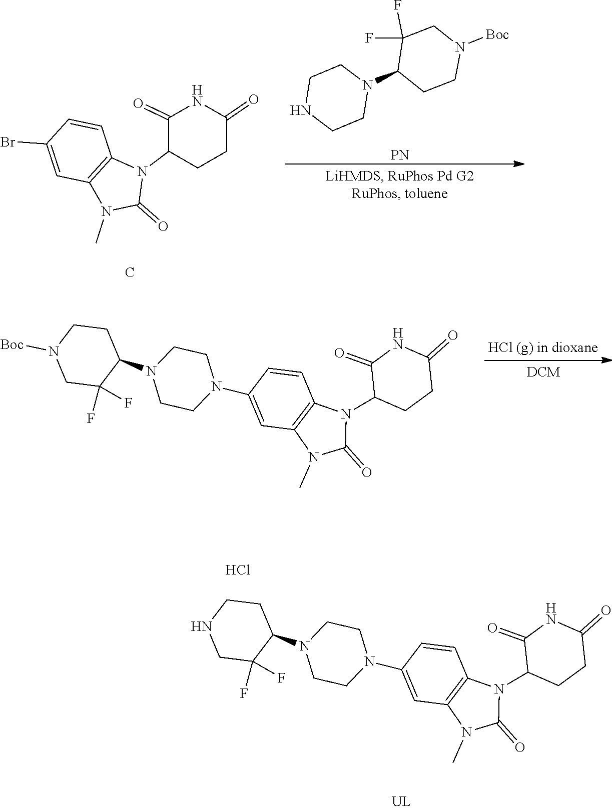

Step 1—Tert-butyl (4R)-4-{4-[1-(2,6-dioxopiperidin-3-yl)-3-methyl-2-oxo-1,3-benzodiazol-5-yl]piperazin-1-yl}-3,3-difluoropiperidine-1-carboxylate. To a stirred mixture of tert-butyl (4R)-3,3-difluoro-4-(piperazin-1-yl)piperidine-1-carboxylate (10 g, 30 mmol, Intermediate PN) and 3-(5-bromo-3-methyl-2-oxo-1,3-benzodiazol-1-yl)piperidine-2,6-dione (11.07 g, 32.75 mmol, Intermediate C) in Toluene (100 mL) were added RuPhos (1.53 g, 3.28 mmol) and RuPhos-PdCl-2nd G (2.55 g, 3.28 mmol) at rt under nitrogen atmosphere. To the above mixture was added 1 M LiHMDS (147.36 mL, 147.36 mmol) in dioxane dropwise over 15 min at rt. The resulting mixture was stirred for an additional 2 h at 80° C. On completion, the reaction mixture was cooled to rt and acidified to pH=4 by formic acid. The resulting mixture was concentrated under reduced pressure. The residue was purified by reverse phase Flash chromatography (Column: WelFlash™ C18-I, 20-40 μm, 330 g; Eluent A: Water (plus 10 mmol/L NH₄HCO₃); Eluent B: ACN; Gradient: 25%-55% B in 25 min; Flow rate: 80 mL/min; Detector: 254 nm; desired fractions were collected at 47% B and concentrated under reduced pressure) to afford the title compound (8 g, 43% yield) as a brown solid. LC/MS (ESI, m/z): [(M+H)]⁺=563.3.

Step 2—3-(5-{4-[(4R)-3,3-difluoropiperidin-4-yl]piperazin-1-yl}-3-methyl-2-oxo-1,3-benzodiazol-1-yl)piperidine-2,6-dione hydrochloride. To a stirred mixture of tert-butyl (4R)-4-{4-[1-(2,6-dioxopiperidin-3-yl)-3-methyl-2-oxo-1,3-benzodiazol-5-yl]piperazin-1-yl}-3,3-difluoropiperidine-1-carboxylate (8 g, 14 mmol) in DCM (100 mL) was added 4 M HCl (gas) in 1,4-dioxane (42.66 mL) at rt. The resulting mixture was stirred for 2 h at rt. On completion, the mixture was concentrated under reduced pressure. The residue was triturated with Et₂O to afford the tile compound (7 g) as a white solid. LC/MS (ESI, m/z): [(M+H)]⁺=463.3.

### 1-(2-Chloro-5-(9-(3,3-difluoropiperidin-4-yl)-3,9-diazaspiro[5.5]undecane-3-carbonyl)phenyl)dihydropyrimidine-2,4(1H,3H)-dione (Intermediate UM)

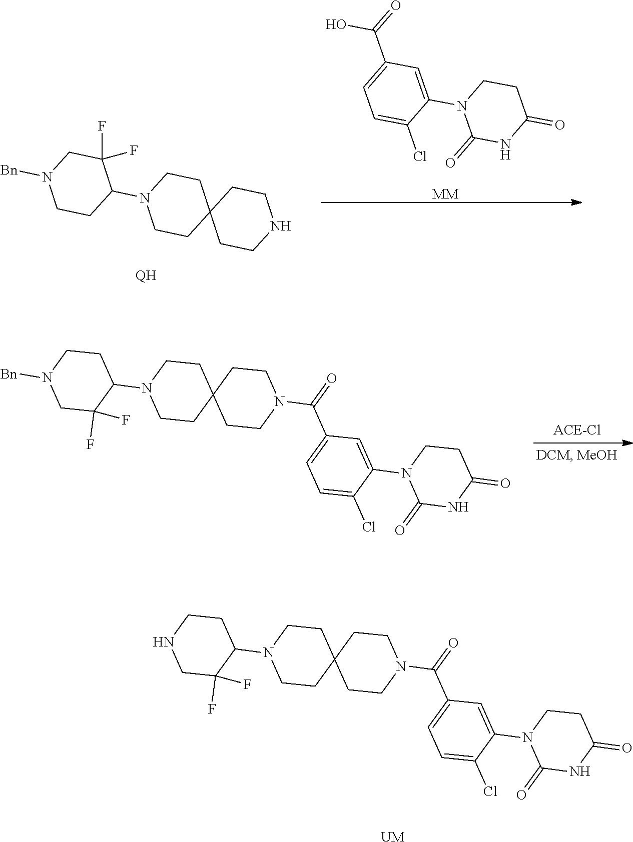

Step 1—1-(5-(9-(1-Benzyl-3,3-difluoropiperidin-4-yl)-3,9-diazaspiro[5.5]undecane-3-carbonyl)-2-chlorophenyl)dihydropyrimidine-2,4(1H,3H)-dione. To a stirred solution of 4-chloro-3-(2,4-dioxo-1,3-diazinan-1-yl)benzoic acid (300 mg, 1.12 mmol, Intermediate MM) in DMA (2 mL) was added DIEA (433 mg, 3.351 mmol) and HATU (636.91 mg, 1.675 mmol) in portions at rt. To the above mixture was added 3-(1-benzyl-3,3-difluoropiperidin-4-yl)-3,9-diazaspiro[5.5]undecane (487 mg, 1.340 mmol, Intermediate QH) in portions at rt. The resulting mixture was stirred for an additional 1 h at rt. On completion, the mixture was purified directly by reverse phase flash chromatography (WelFlash™ C18-I, 20-40 μm, 330 g; Eluent A: Water (plus 10 mmol/L NH₄HCO₃); Eluent B: ACN; Gradient: 30%-60% B in 25 min; Flow rate: 80 mL/min; Detector: 220/254 nm; desired fractions were collected at 58% B and concentrated under reduced pressure) to afford the title compound (240 mg, 35% yield) as a reddish solid. LC/MS (ESI, m/z): [(M+1)]⁺=614.4.

Step 2—1-(2-Chloro-5-(9-(3,3-difluoropiperidin-4-yl)-3,9-diazaspiro[5.5]undecane-3-carbonyl)phenyl)dihydropyrimidine-2,4(1H,3H)-dione. To a stirred solution of 1-{5-[9-(1-benzyl-3,3-difluoropiperidin-4-yl)-3,9-diazaspiro[5.5]undecane-3-carbonyl]-2-chlorophenyl}-1,3-diazinane-2,4-dione (190 mg, 0.309 mmol) in DCM (5 mL) was added 1-chloroethyl carbonochloridate (221 mg, 1.545 mmol) dropwise at 0° C. under air atmosphere. The resulting mixture was stirred for 3 h at 50° C. Next, the mixture was allowed to cool down to rt. The resulting mixture was concentrated under reduced pressure. The residue was dissolved in MeOH (5 mL). The resulting mixture was stirred for 1 h at 70° C. under air atmosphere. On completion, the mixture was allowed to cool down to rt. The mixture was concentrated under reduced pressure. The residue product was purified by reverse phase flash chromatography (WelFlash™ C18-I, 20-40 μm, 330 g; Eluent A: Water (plus or 10 mmol/L NH₄HCO₃); Eluent B: ACN; Gradient: 25%-55% B in 25 min; Flow rate: 80 mL/min; Detector: 220/254 nm; desired fractions were collected at 38% B and concentrated under reduced pressure) to afford the title compound (160 mg, 99% yield) as a white solid. LC/MS (ESI, m/z): [(M+1)]⁺=524.2.

### 3-(5-(3,3-Difluoro-[4,1′:4′,4″-terpiperidin]-1″-yl)-3-methyl-2-oxo-2,3-dihydro-1H-benzo[d]imidazol-1-yl)piperidine-2,6-dione (Intermediate UN)

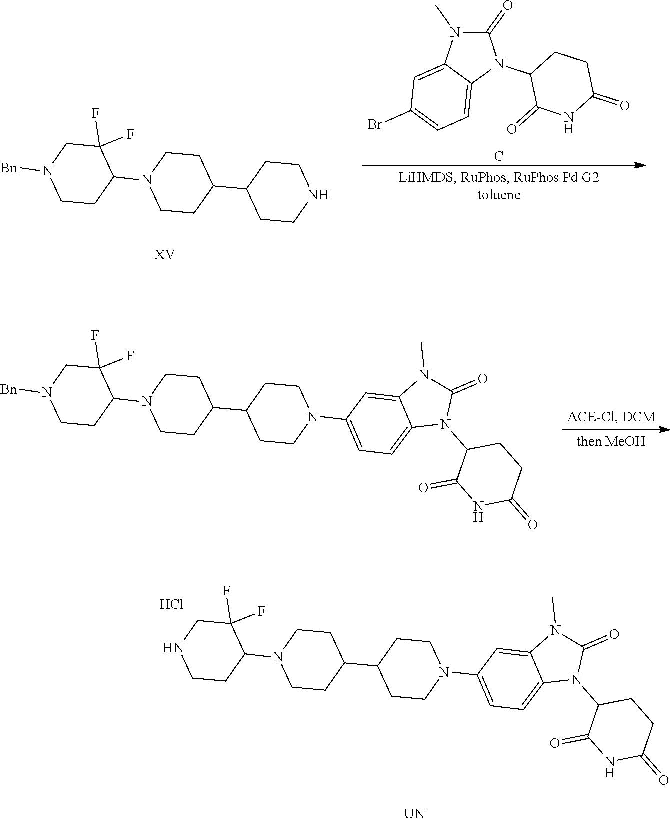

Step 1—3-(5-(1-benzyl-3,3-difluoro-[4,1′:4′,4″-terpiperidin]-1″-yl)-3-methyl-2-oxo-2,3-dihydro-1H-benzo[d]imidazol-1-yl)piperidine-2,6-dione. To a stirred mixture of 1-benzyl-3,3-difluoro-4,1′:4′,4″-terpiperidine hydrochloride (1.50 g, 3.97 mmol, Intermediate XV) and 3-(5-bromo-3-methyl-2-oxo-2,3-dihydro-TH-benzo[d]imidazol-1-yl)piperidine-2,6-dione (1.61 g, 4.77 mmol, Intermediate C) in toluene (20 mL) were added RuPhos (0.19 g, 0.40 mmol) and RuPhos-PdCl-2nd G (0.31 g, 0.40 mmol) at rt under N₂ atmosphere. The resulting mixture was stirred for 15 min at it under nitrogen atmosphere. To the above mixture was added LiHMDS (23.84 mL, 23.87 mmol) dropwise at rt. The resulting mixture was stirred for an additional 2 h at 80° C. On completion, the reaction was cooled to rt and quenched with sat. NH4Cl (aq.) at 0° C. The resulting mixture was filtered, and the filter cake was washed with EtOAc (3×10 mL). The filtrate was concentrated under reduced pressure. The residue was purified by reverse flash chromatography (column, WelFlash™ C18-I, 20-40 μm, 330 g; Eluent A: Water (plus 10 mmol/L NH₄CO₃); Eluent B: ACN, 40% to 80% gradient in 30 min; Flow rate: 80 mL/min; detector: UV 220/254 nm, desired fractions were collected at 69% B and concentrated under reduced pressure) to afford the title compound (1.2 g, 42% yield) as a yellow solid. LC/MS (ESI, m/z): [(M+H)]-=635.3.

Step 2—3-(5-(3,3-difluoro-[4,1′:4′,4″-terpiperidin]-1″-yl)-3-methyl-2-oxo-2,3-dihydro-1H-benzo[d]imidazol-1-yl)piperidine-2,6-dione hydrochloride. To a stirred mixture of 3-(5-(1-benzyl-3,3-difluoro-[4,1′:4′,4″-terpiperidin]-1″-yl)-3-methyl-2-oxo-2,3-dihydro-1H-benzo[d]imidazol-1-yl)piperidine-2,6-dione (300 mg, 0.47 mmol) in DCM (6 mL) was added 1-chloroethyl carbonochloridate (338 mg, 2.36 mmol) dropwise at 0° C. under N₂ atmosphere. The resulting mixture was stirred for 3 h at 50° C. under nitrogen atmosphere. Then the mixture was concentrated under reduced pressure. To the above mixture was added MeOH (6 mL) at rt. The resulting mixture was stirred for an additional 1 h at 70° C. The precipitated solids were collected by filtration and washed with EtOAc (3×10 mL) to afford the title compound (240 mg, 93% yield) as a white solid. LC/MS (ESI, m/z): [(M+H)]⁺=545.3.

### 5-(2,4-Dioxotetrahydropyrimidin-1(2H)-yl)-6-methoxynicotinic acid (Intermediate UO)

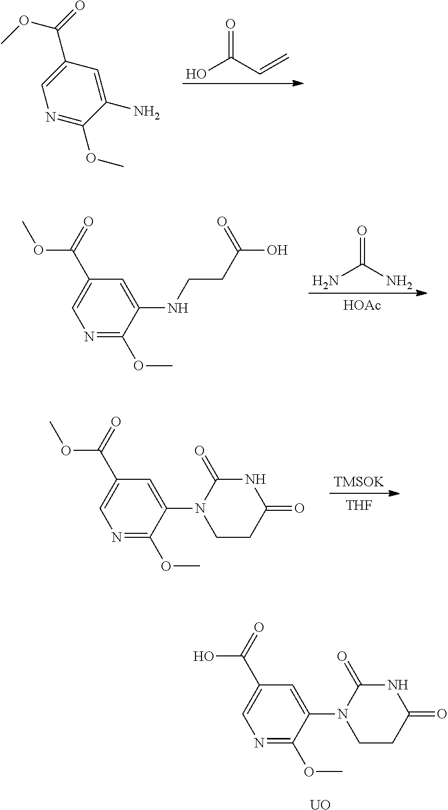

Step 1—3-((2-Methoxy-5-(methoxycarbonyl)pyridin-3-yl)amino)propanoic acid. A mixture of methyl 5-amino-6-methoxynicotinate (CAS #59237-50-2) (2.00 g, 10.98 mmol) in acrylic acid (1.60 g, 22.0 mmol) was stirred for 16 h at 100° C. The resulting mixture was diluted with water (50 mL). The resulting mixture was extracted with EtOAc (3×100 mL), and dried over anhydrous Na₂SO₄. After filtration, the filtrate was concentrated under reduced pressure to afford the title compound (3.4 g) as an off-white solid. LC/MS (ESI, m/z): [(M+1)]⁺=255.1.

Step 2—Methyl 5-(2,4-dioxotetrahydropyrimidin-1(2H)-yl)-6-methoxynicotinate. To a solution of 3-((2-methoxy-5-(methoxycarbonyl)pyridin-3-yl)amino)propanoic acid (3.20 g, 12.6 mmol) in AcOH (33 mL) was urea (5.29 g, 88.1 mmol). The resulting mixture was stirred for 16 h at 110° C. The mixture was purified directly by reversed phase flash chromatography (Column: Spherical C18, 20˜40 μm, 330 g; Mobile Phase A: Water (plus 0.1% HCOOH), Mobile Phase B: ACN; Flow rate: 80 mL/min; Gradient (B %): 3%˜3%, 4 min; 3%˜25%, 20 min; 25%-95%; 2 min; 95%, 5 min; Detector: 204 nm; the fractions containing desired product were collected at 20% B and concentrated under reduced pressure) to afford the title compound (1.88 g, 53% yield) as a yellow solid. LC/MS (ESI, m/z): [(M+1)]⁺=280.1.

Step 3—5-(2,4-Dioxotetrahydropyrimidin-1(2H)-yl)-6-methoxynicotinic acid. A solution of methyl 5-(2,4-dioxotetrahydropyrimidin-1(2H)-yl)-6-methoxynicotinate (100 mg, 0.358 mmol) and potassium trimethylsilanolate (229 mg, 1.79 mmol) in THF (2 mL) was stirred for 1 h at rt. On completion, the mixture was acidified to pH 5 with 3 N HCl (aq.). The mixture was purified by reversed phase flash chromatography (Column: Spherical C18, 20˜40 μm, 40 g; Mobile Phase A: Water (plus 0.1% HCOOH), Mobile Phase B: MeCN; Flow rate: 30 mL/min; Gradient (B %): 3%˜3%, 5 min; 3%˜20%, 20 min; 20%-95%; 2 min; 95%, 5 min; Detector: 254 nm; the fractions containing desired product were collected at 5% B and concentrated under reduced pressure) to afford the title compound (55 mg, 49% yield) as a white solid. LC/MS (ESI, m/z): [(M+1)]m=266.2.

### 2-(1-Benzyl-3,3-difluoropiperidin-4-yl)-2,7-diazaspiro[3.5]nonane (Intermediate UP)

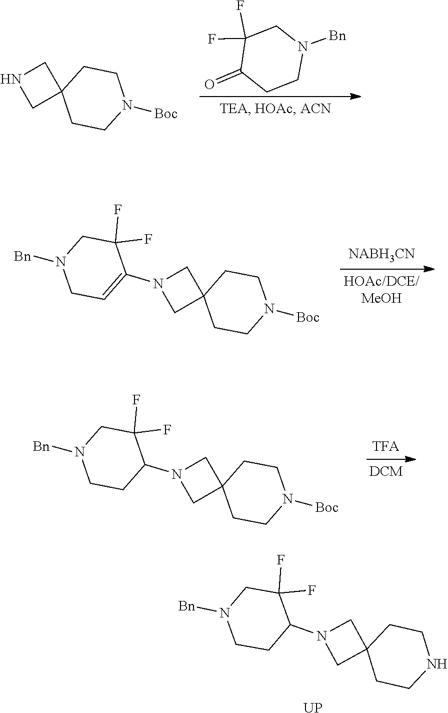

Step 1—Tert-butyl 2-(1-benzyl-3,3-difluoro-1,2,3,6-tetrahydropyridin-4-yl)-2,7-diazaspiro[3.5]nonane-7-carboxylate. To a stirred solution of tert-butyl 2,7-diazaspiro[3.5]nonane-7-carboxylate (10.00 g, 44.18 mmol) and TEA (6.71 g, 66.3 mmol) in ACN (150 mL) was added 1-benzyl-3,3-difluoropiperidin-4-one (19.90 g, 88.37 mmol) and HOAc (7.96 g, 132.55 mmol) in turns at rt under nitrogen atmosphere. The resulting mixture was stirred for 16 h at 110° C. under nitrogen atmosphere. On completion, the mixture was cooled to rt and basified to pH 8 with saturated NaHCO₃ (aq.). The resulting mixture was diluted with water (300 mL) and extracted with EtOAc (3×300 mL). The combined organic layers were washed with water (300 mL), and dried over anhydrous Na₂SO₄. After filtration, the filtrate was concentrated under reduced pressure to give the title compound (19.00 g, 99% yield) as a yellow solid. LC/MS (ESI, m/z): [(M+1)]⁺=434.3.

Step 2—Tert-butyl 2-(1-benzyl-3,3-difluoropiperidin-4-yl)-2,7-diazaspiro[3.5]nonane-7-carboxylate. To a stirred solution of tert-butyl 2-(1-benzyl-3,3-difluoro-1,2,3,6-tetrahydropyridin-4-yl)-2,7-diazaspiro[3.5]nonane-7-carboxylate (22.00 g, 50.74 mmol) and HOAc (11 mL) in DCE (110 mL) and MeOH (110 mL) was added NaBH₃CN (15.94 g, 253.7 mmol) in portions at rt under nitrogen atmosphere. The resulting mixture was stirred for 16 h at rt under nitrogen atmosphere. On completion, the mixture was concentrated under vacuum. The residue was purified by silica gel column chromatography, eluted with CH₂Cl₂/EA (1:1) to afford the title compound (9.20 g, 41% yield) as a yellow solid. LC/MS (ESI, m/z): [(M+1)]⁺=436.3.

Step 3—2-(1-Benzyl-3,3-difluoropiperidin-4-yl)-2,7-diazaspiro[3.5]nonane trifluoroacetate. To a stirred solution of tert-butyl 2-(1-benzyl-3,3-difluoropiperidin-4-yl)-2,7-diazaspiro[3.5]nonane-7-carboxylate (3.40 g, 7.80 mmol) in DCM (45 mL) was added TFA (17 mL) dropwise at rt under nitrogen atmosphere. The resulting mixture was stirred for 1 h at it under nitrogen atmosphere. On completion, the mixture was concentrated under vacuum. The residue was purified by trituration with Et₂O (100 mL) to afford the title compound (2.00 g, 76% yield) as a white solid. LC/MS (ESI, m/z): [(M+1)]=336.2.

### 3-(4-(2-(3,3-difluoropiperidin-4-yl)-2,7-diazaspiro[3.5]nonan-7-yl)-3-methyl-2-oxo-2,3-dihydro-1H-benzo[d]imidazol-1-yl)piperidine-2,6-dione (Intermediate UQ)

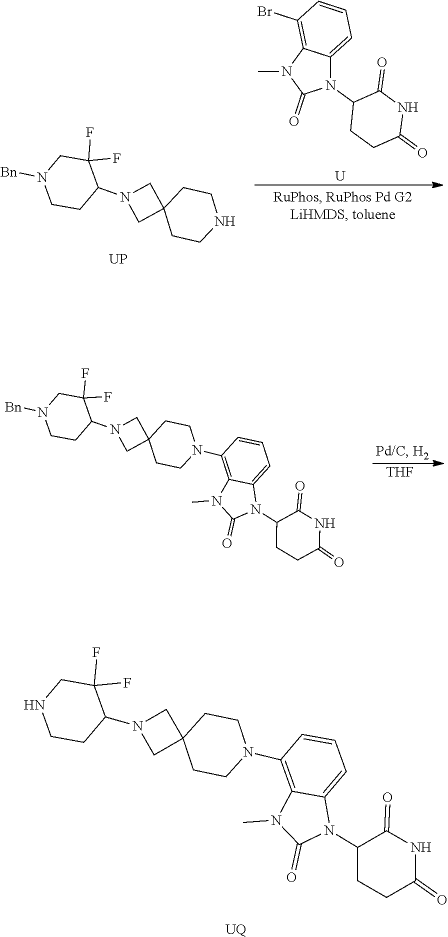

Step 1—3-(4-(2-(1-Benzyl-3,3-difluoropiperidin-4-yl)-2,7-diazaspiro[3.5]nonan-7-yl)-3-methyl-2-oxo-2,3-dihydro-1H-benzo[d]imidazol-1-yl)piperidine-2,6-dione. To a stirred solution of 2-(1-benzyl-3,3-difluoropiperidin-4-yl)-2,7-diazaspiro[3.5]nonane trifluoroacetate (600 mg, 1.79 mmol, Intermediate UP) and 3-(4-bromo-3-methyl-2-oxo-1,3-benzodiazol-1-yl)piperidine-2,6-dione (604 mg, 1.79 mmol, Intermediate U) in toluene (10 mL) was added RuPhos (83 mg, 0.179 mmol) and RuPhos-PdCl-2nd G (139 mg, 0.179 mmol) in turns at it under nitrogen atmosphere. The resulting mixture was stirred for 1 h at 80° C. under nitrogen atmosphere. On completion, the mixture was cooled to rt and concentrated under vacuum. The residue was purified by silica gel column chromatography, eluted with CH₂C12/EA (1:1) to afford the title compound (1.00 g, 94% yield) as a yellow oil. LC/MS (ESI, m/z): [(M+1)]⁺=593.2.

Step 2—3-(4-(2-(3,3-difluoropiperidin-4-yl)-2,7-diazaspiro[3.5]nonan-7-yl)-3-methyl-2-oxo-2,3-dihydro-TH-benzo[d]imidazol-1-yl)piperidine-2,6-dione. To a stirred solution of 3-(4-(2-(1-benzyl-3,3-difluoropiperidin-4-yl)-2,7-diazaspiro[3.5]nonan-7-yl)-3-methyl-2-oxo-2,3-dihydro-1H-benzo[d]imidazol-1-yl)piperidine-2,6-dione (300 mg, 0.506 mmol) in THF (48 mL) was added Pd/C (5 mg, 0.051 mmol) in portions at rt under nitrogen atmosphere. The reaction system was degassed under vacuum and purged with H₂ several times, then it was hydrogenated under H₂ balloon (˜1 atm) at 25° C. for 2 h. After completion of the reaction, Pd/C was filtered off through celite and the filter cake was washed with THF (3×300 mL). The corresponding filtrate was concentrated under reduced pressure to provide the title compound (55 mg, 21% yield) as a white solid. LC/MS (ESI, m/z): [(M+1)]m=503.1 Tert-butyl 2-{[2-(2,3-dihydro-1H-indol-4-yl)pyrimidin-5-yl]methyl}-2,7-diazaspiro[3.5]nonane-7-carboxylate (Intermediate UR)

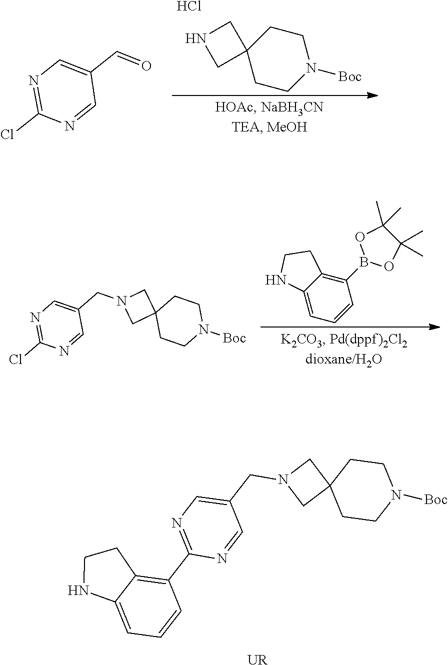

Step 1—Tert-butyl 2-[(2-chloropyrimidin-5-yl)methyl]-2,7-diazaspiro[3.5]nonane-7-carboxylate. To a stirred mixture of tert-butyl 2,7-diazaspiro[3.5]nonane-7-carboxylate Hydrochloride (3.68 g, 14.0 mmol) and TEA (4.26 g, 42.1 mmol) in MeOH (30 mL) was added 2-chloropyrimidine-5-carbaldehyde (2 g, 14 mmol) and HOAc (4.02 mL, 70.2 mmol) at rt under nitrogen atmosphere. The resulting mixture was stirred for 40 min at rt under nitrogen atmosphere. To the above mixture was added NaBH₃CN (1.76 g, 28.1 mmol) and stirred for additional 2 h at rt. On completion, the resulting mixture was concentrated under vacuum. The residue was purified by silica gel column chromatography, eluted with CH₂C2/EA (5:1˜1:10), to afford the title compound (3.4 g, 69% yield) as a white solid. LC/MS (ESI, m/z): [(M+H)]=353.2.

Step 2—Tert-butyl 2-{[2-(2,3-dihydro-1H-indol-4-yl)pyrimidin-5-yl]methyl}-2,7-diazaspiro[3.5]nonane-7-carboxylate. To a stirred mixture of tert-butyl 2-[(2-bromopyrimidin-5-yl)methyl]-2,7-diazaspiro[3.5]nonane-7-carboxylate (500 mg, 1.26 mmol) and 4-(4,4,5,5-tetramethyl-1,3,2-dioxaborolan-2-yl)-2,3-dihydro-1H-indole (370.18 mg, 1.510 mmol) in dioxane (6 mL) and H₂O (1 mL) was added K₂CO₃ (521.77 mg, 3.774 mmol) and Pd(dppf)Cl₂CH₂Cl₂ (102.52 mg, 0.126 mmol) at rt under nitrogen atmosphere. The resulting mixture was stirred for 2 h at 80° C. under nitrogen atmosphere. On completion, the mixture was cooled to rt and concentrated under reduced pressure. The residue was purified by silica gel column chromatography, eluted with PE/EA (5:1-1:5), to afford the title compound (300 mg, 55% yield) as a white solid. LC/MS (ESI, m/z): [(M+H)]⁺=436.2.

### 6-[4-(5-{2,7-diazaspiro[3.5]nonan-2-ylmethyl}pyrimidin-2-yl)-2,3-dihydroindol-1-yl]-N-[(1R,2S)-2-fluorocyclopropyl]-8-(methylamino)imidazo[1,2-b]pyridazine-3-carboxamide (Intermediate US)

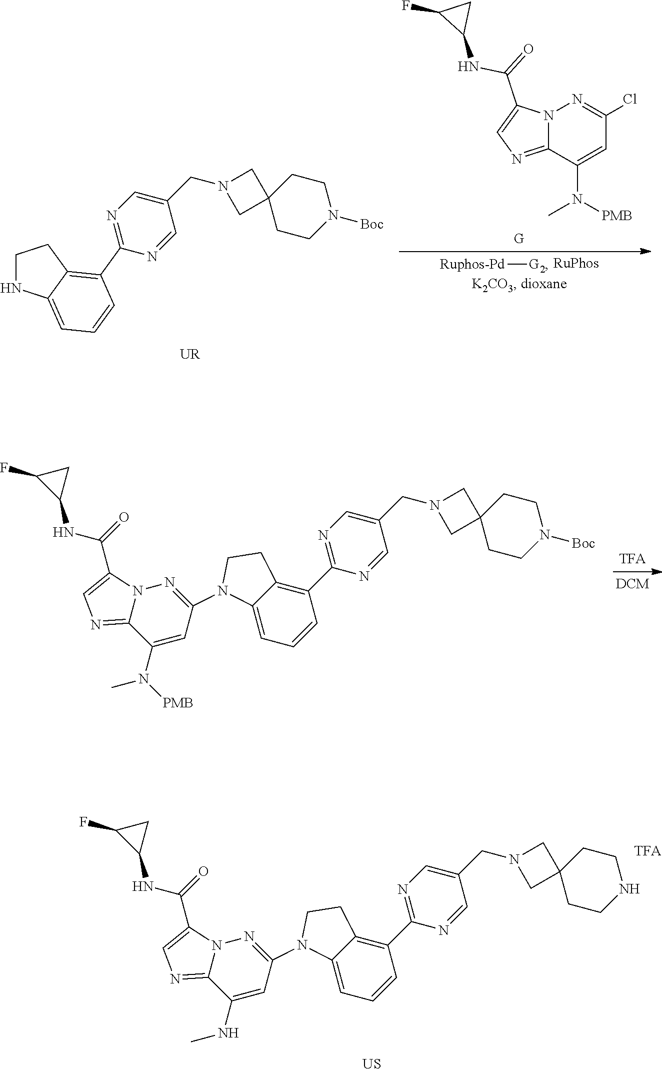

Step 1—Tert-butyl 2-({2-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyrimidin-5-yl}methyl)-2,7-diazaspiro[3.5]nonane-7-carboxylate. To a stirred mixture of tert-butyl 2-{[2-(2,3-dihydro-1H-indol-4-yl)pyrimidin-5-yl]methyl}-2,7-diazaspiro[3.5]nonane-7-carboxylate (250 mg, 0.574 mmol, Intermediate UR) and 6-chloro-N-[(1R,2S)-2-fluorocyclopropyl]-8-{[(4-methoxyphenyl)methyl](methyl)amino}imidazo[1,2-b]pyridazine-3-carboxamide (231.79 mg, 0.574 mmol, Intermediate G) in dioxane (6 mL) were added K₂CO₃ (237.97 mg, 1.722 mmol), RuPhos (53.57 mg, 0.115 mmol) and RuPhos-PdCl-2nd G (89.28 mg, 0.115 mmol) at rt under nitrogen atmosphere. The resulting mixture was stirred for an additional 2 h at 90° C. under nitrogen atmosphere. On completion, the mixture was cooled to rt and concentrated under reduced pressure. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 75%-95% B in 25 min; Flow rate: 60 mL/min; Detector: 220/254 nm; desired fractions were collected at 84% B and concentrated under reduced pressure) to afford the title compound (327 mg, 71% yield) as a yellow solid. LC/MS (ESI, m/z): [(M+H)]⁺=803.5.

Step 2—6-[4-(5-{2,7-diazaspiro[3.5]nonan-2-ylmethyl}pyrimidin-2-yl)-2,3-dihydroindol-1-yl]-N-[(1R,2S)-2-fluorocyclopropyl]-8-(methylamino)imidazo[1,2-b]pyridazine-3-carboxamide trifluoroacetate. To a stirred solution of tert-butyl 2-({2-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyrimidin-5-yl}methyl)-2,7-diazaspiro[3.5]nonane-7-carboxylate (327 mg, 0.407 mmol) in DCM (6 mL) was added TFA (2 mL) at rt. The resulting mixture was stirred for an additional 1 h at rt. On completion, the mixture was concentrated under reduced pressure. The residue was purified by trituration with Et₂O (20 mL) to afford the title compound (350 mg) as an off-white solid. LC/MS (ESI, m/z): [(M+H)]⁺=583.3.

### Tert-butyl 2-[(6-chloropyridazin-3-yl)methyl]-2,7-diazaspiro[3.5]nonane-7-carboxylate (Intermediate UT)

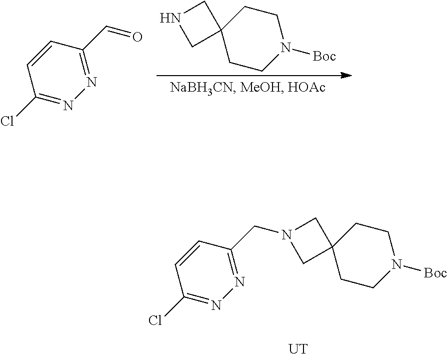

To a stirred mixture of 6-chloropyridazine-3-carbaldehyde (950 mg, 6.67 mmol) and tert-butyl 2,7-diazaspiro[3.5]nonane-7-carboxylate (1508.38 mg, 6.665 mmol, CAS #896464-16-7) in MeOH (10 mL) was added AcOH (1.91 mL, 33.3 mmol) at rt. The resulting mixture was stirred for additional 20 min at rt. To the above mixture was added NaBH₃CN (1256.45 mg, 19.995 mmol) at rt. The resulting mixture was stirred for an additional 2 h at 50° C. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 40%-60% B in 25 min; Flow rate: 80 mL/min; Detector: 220/254 nm; desired fractions were collected at 55% B and concentrated under reduced pressure) to afford the title compound (560 mg, 24% yield) as a yellow oil. LC/MS (ESI, m/z): [(M+H)]⁺=353.1.

### 6-[4-(6-{2,7-diazaspiro[3.5]nonan-2-ylmethyl}pyridazin-3-yl)-2,3-dihydroindol-1-yl]-N-[(1R,2S)-2-fluorocyclopropyl]-8-(methylamino)imidazo[1,2-b]pyridazine-3-carboxamide (Intermediate UU)

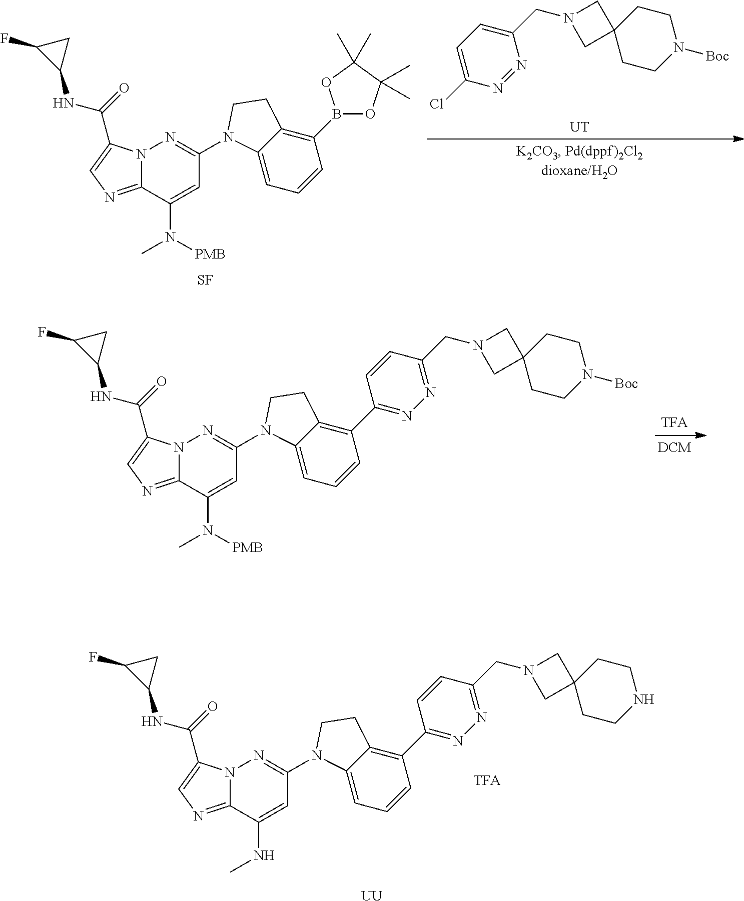

Step 1—Tert-butyl 2-({6-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyridazin-3-yl}methyl)-2,7-diazaspiro[3.5]nonane-7-carboxylate. To a stirred mixture of tert-butyl 2-[(6-chloropyridazin-3-yl)methyl]-2,7-diazaspiro[3.5]nonane-7-carboxylate (560 mg, 1.59 mmol. Intermediate UT) and N-[(1R,2S)-2-fluorocyclopropyl]-8-{[(4-methoxyphenyl)methyl](methyl)amino}-6-[4-(4,4,5,5-tetramethyl-1,3,2-dioxaborolan-2-yl)-2,3-dihydroindol-1-yl]imidazo[1,2-b]pyridazine-3-carboxamide (972.07 mg, 1.587 mmol, Intermediate SF) in dioxane (6 mL) and H₂O (3 mL) was added K₂CO₃ (658.01 mg, 4.761 mmol) at rt under nitrogen atmosphere. To the above mixture was added Pd(dppf)Cl₂·CH2Cl₂ (129.28 mg, 0.159 mmol) at rt under nitrogen atmosphere. The resulting mixture was stirred for additional 16 h at 90° C. under nitrogen atmosphere. On completion, the mixture was cooled to rt and diluted with water (30 mL) and extracted with EtOAc (3×50 mL). The combined organic layers were washed with brine (1×50 mL), and dried over anhydrous Na₂SO₄. After filtration, the filtrate was concentrated under reduced pressure. The residue was purified by reverse phase flash chromatography (Column: WelFlash™ C18-I, 20-40 μm, 330 g; Eluent A: Water (plus 10 mmol/L FA); Eluent B: ACN; Gradient: 40%-55% B in 25 min; Flow rate: 80 mL/min; Detector: 220/254 nm; desired fractions were collected at 44% B and concentrated under reduced pressure) to afford the title compound (725 mg, 57% yield) as a yellow solid. LC/MS (ESI, m/z): [(M+H)]⁺=803.3.

Step 2—6-[4-(6-{2,7-diazaspiro[3.5]nonan-2-ylmethyl}pyridazin-3-yl)-2,3-dihydroindol-1-yl]-N-[(1R,2S)-2-fluorocyclopropyl]-8-(methylamino)imidazo[1,2-b]pyridazine-3-carboxamide trifluoroacetate. To a stirred solution of tert-butyl 2-({6-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyridazin-3-yl}methyl)-2,7-diazaspiro[3.5]nonane-7-carboxylate (725 mg, 0.903 mmol) in DCM (6 mL) was added TFA (2 mL) at rt. The resulting mixture was stirred for 1 h at rt. On completion, the mixture was concentrated under reduced pressure. The residue was purified by trituration with Et₂O (30 mL) to afford the title compound (700 mg) as a yellow solid. LC/MS (ESI, m/z): [(M+H)]⁺=583.3.

### 4-[(E)-2-ethoxyethenyl]-2,3-dihydro-1H-indole (Intermediate UV)

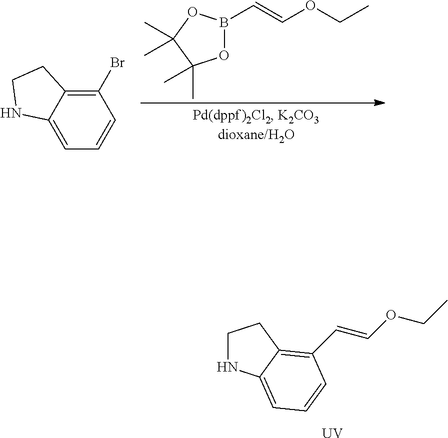

To a stirred solution of 4-bromo-2,3-dihydro-1H-indole (3 g, 15 mmol, CAS #86626-38-2) and 2-[(E)-2-ethoxyethenyl]-4,4,5,5-tetramethyl-1,3,2-dioxaborolane (3.60 g, 18.2 mmol, CAS #1201905-61-4) in dioxane (45 mL) and H₂O (9 mL) were added Pd(dppf)Cl₂ CH₂Cl₂ (1.23 g, 1.51 mmol) and K₂CO₃ (4.19 g, 30.29 mmol) in portions at rt under nitrogen atmosphere. The resulting mixture was stirred for 2 h at 80° C. under nitrogen atmosphere. On completion, the mixture was cooled to rt and concentrated under vacuum. The residue was purified by reverse phase Flash chromatography (Column: WelFlash™ C18-I, 20-40 μm, 330 g; Eluent A: Water (10 mmol/L NH₄HCO₃); Eluent B: ACN; Gradient: 25%-55% B in 40 min; Flow rate: 80 mL/min; Detector: 220/254 nm; desired fractions were collected at 50% B and concentrated under reduced pressure) to afford the title compound (2.7 g, 94% yield) as a yellow oil. LC/MS (ESI, m/z): [(M+1)]⁺=190.2.

### N-((1R,2S)-2-fluorocyclopropyl)-8-(methylamino)-6-(4-(2-oxoethyl)indolin-1-yl)imidazo[1,2-b]pyridazine-3-carboxamide (Intermediate UW)

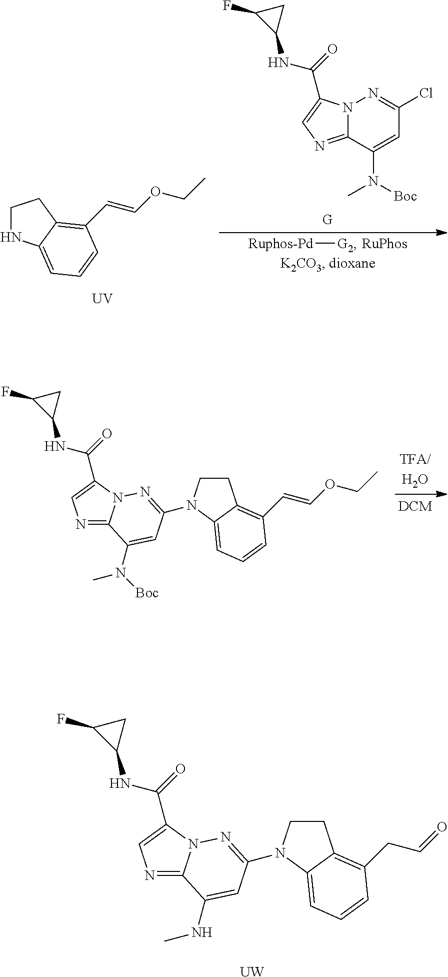

Step 1—Tert-butyl (6-(4-((E)-2-ethoxyvinyl)indolin-1-yl)-3-(((1R,2S)-2-fluorocyclopropyl)carbamoyl)imidazo[1,2-b]pyridazin-8-yl)(methyl)carbamate. To a stirred solution of 4-[(E)-2-ethoxyethenyl]-2,3-dihydro-1H-indole (250 mg, 1.32 mmol, Intermediate UV) and tert-butyl N-(6-chloro-3-{[(R,2S)-2-fluorocyclopropyl]carbamoyl}imidazo[1,2-b]pyridazin-8-yl)-N-methylcarbamate (506 mg, 1.32 mmol, Intermediate G) in dioxane (4 mL) were added RuPhos (61 mg, 0.132 mmol) and K₂CO₃ (547 mg, 3.96 mmol) in turns at rt under nitrogen atmosphere. The resulting mixture was stirred for 2 h at 100° C. under nitrogen atmosphere. On completion, the mixture was cooled to rt and concentrated under vacuum. The residue was purified by silica gel column chromatography, eluted with EA, to afford the title compound (250 mg, 35% yield) as a yellow solid. LC/MS (ESI, m/z): [(M+1)]=537.2.

Step 2—N-((1R,2S)-2-fluorocyclopropyl)-8-(methylamino)-6-(4-(2-oxoethyl)indolin-1-yl)imidazo[1,2-b]pyridazine-3-carboxamide. To a stirred solution of tert-butyl (6-(4-((E)-2-ethoxyvinyl)indolin-1-yl)-3-(((1R,2S)-2-fluorocyclopropyl)carbamoyl)imidazo[1,2-b]pyridazin-8-yl)(methyl)carbamate (120 mg, 0.224 mmol) in H₂O (1 mL) was added THF (1 mL) in portions at rt under nitrogen atmosphere and the mixture was stirred for 1 h at rt under nitrogen atmosphere. On completion, the mixture was diluted with water (100 mL) and extracted with EtOAc (3×50 mL). The combined organic layers were washed with brine, and dried over anhydrous Na₂SO₄. After filtration, the filtrate was concentrated under reduced pressure to give the title compound (90 mg, 98% yield) as a yellow oil. LC/MS (ESI, m/z): [(M+1)]⁺=409.1.

### Tert-butyl 2-[(5-chloropyrazin-2-yl)methyl]-2,7-diazaspiro[3.5]nonane-7-carboxylate (Intermediate UX)

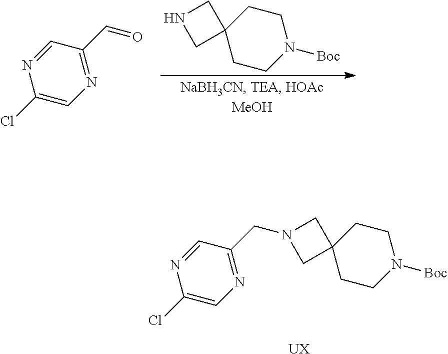

To a stirred solution of tert-butyl 2,7-diazaspiro[3.5]nonane-7-carboxylate (1.59 g, 7.016 mmol, CAS #896464-16-7) in MeOH (10 mL) was added TEA (2.93 mL, 21.1 mmol) at rt and the mixture was stirred for 5 min. To the above mixture was added 5-chloropyrazine-2-carbaldehyde (1 g, 7 mmol) and AcOH (2.01 mL, 35.080 mmol) at rt and the mixture was stirred for additional 30 min at rt. Next, NaBH₃CN (1.32 g, 21.048 mmol) was added at rt then the mixture was stirred for an additional 2 h at 50° C. On completion, the mixture was cooled to rt and concentrated under reduced pressure. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 50%-70% B in 25 min; Flow rate: 80 mL/min; Detector: 220/254 nm; desired fractions were collected at 60% B and concentrated under reduced pressure) to afford the title compound (1.6 g, 65% yield) as a yellow oil. LC/MS (ESI, m/z): [(M+H)]⁺=353.3.

### 6-[4-(5-{2,7-Diazaspiro[3.5]nonan-2-ylmethyl}pyrazin-2-yl)-2,3-dihydroindol-1-yl]-N-[(1R,2S)-2-fluorocyclopropyl]-8-(methylamino)imidazo[1,2-b]pyridazine-3-carboxamide (Intermediate UY)

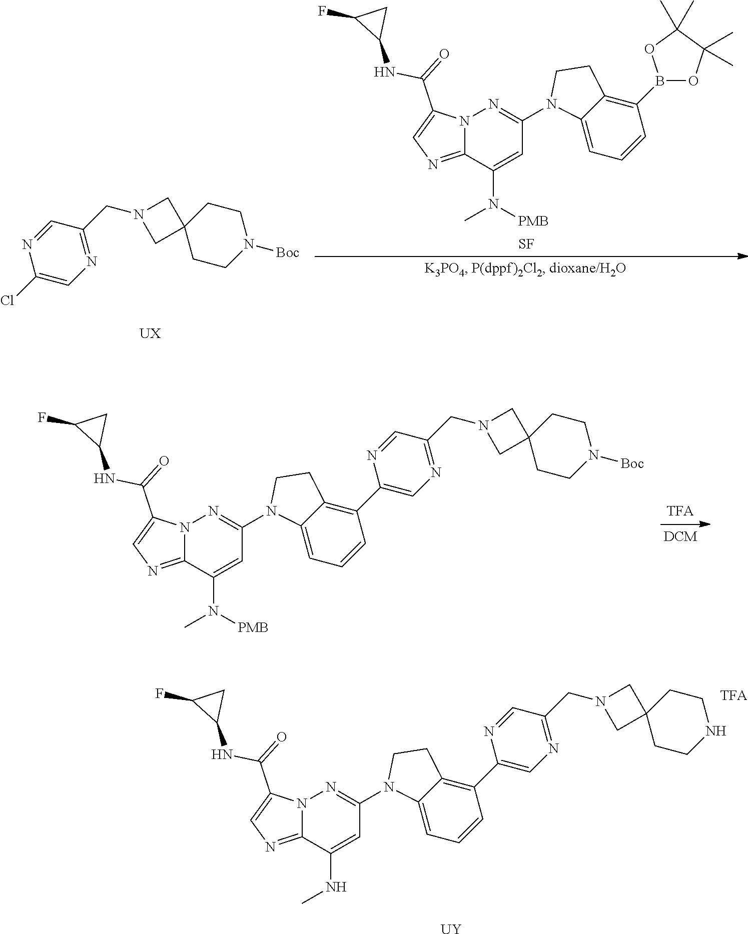

Step 1—Tert-butyl 2-({5-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyrazin-2-yl}methyl)-2,7-diazaspiro[3.5]nonane-7-carboxylate. To a stirred mixture of tert-butyl 2-[(5-chloropyrazin-2-yl)methyl]-2,7-diazaspiro[3.5]nonane-7-carboxylate (800 mg, 2.27 mmol, Intermediate UX) and N-[(1R,2S)-2-fluorocyclopropyl]-8-{[(4-methoxyphenyl)methyl](methyl)amino}-6-[4-(4,4,5,5-tetramethyl-1,3,2-dioxaborolan-2-yl)-2,3-dihydroindol-1-yl]imidazo[1,2-b]pyridazine-3-carboxamide (1527.54 mg, 2.494 mmol, Intermediate SF) in dioxane (10 mL) and H₂O (2 mL) were added K₂CO₃ (11.75 mg, 0.084 mmol) and Pd(dppf)Cl₂·CH₂Cl₂(184.69 mg, 0.227 mmol) at rt under nitrogen atmosphere. The resulting mixture was stirred for 2 h at 90° C. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 70%-90% B in 25 min; Flow rate: 80 mL/min; Detector: 220/254 nm; desired fractions were collected at 78% B and concentrated under reduced pressure) to afford the title compound (440 mg, 24% yield) as an off-white solid. LC/MS (ESI, m/z): [(M+H)]⁺=803.5.

Step 2—6-[4-(5-{2,7-Diazaspiro[3.5]nonan-2-ylmethyl}pyrazin-2-yl)-2,3-dihydroindol-1-yl]-N-[(1R,2S)-2-fluorocyclopropyl]-8-(methylamino)imidazo[1,2-b]pyridazine-3-carboxamide trifluoroacetate To a stirred solution of tert-butyl 2-({5-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyrazin-2-yl}methyl)-2,7-diazaspiro[3.5]nonane-7-carboxylate (440 mg, 0.548 mmol) in DCM (6 mL) was added TFA (2 mL) at rt and the mixture was for 1 h at it. On completion, the mixture was concentrated under reduced pressure. The residue was purified by trituration with Et₂O (20 mL) to afford the title compound (560 mg) as an off-white solid. LC/MS (ESI, m/z): [(M+H)]=583.5.

### 3-(5-(4-((R)-3,3-difluoropiperidin-4-yl)piperazin-1-yl)-4-fluoro-3-methyl-2-oxo-2,3-dihydro-1H-benzo[d]imidazol-1-yl)piperidine-2,6-dione (Intermediate UZ)

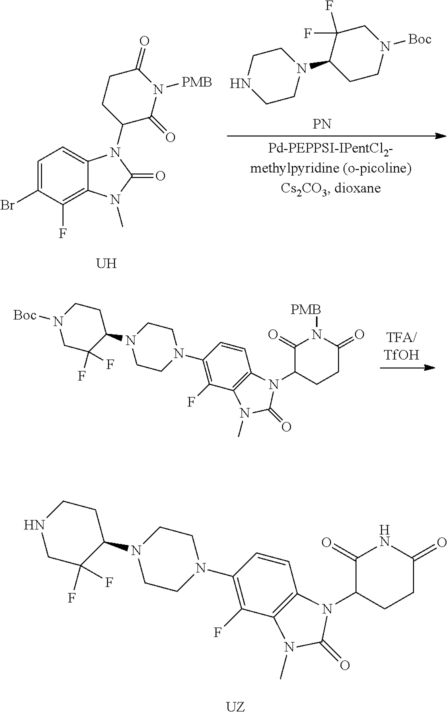

Step 1—Tert-butyl (4R)-3,3-difluoro-4-(4-(4-fluoro-1-(1-(4-methoxybenzyl)-2,6-dioxopiperidin-3-yl)-3-methyl-2-oxo-2,3-dihydro-1H-benzo[d]imidazol-5-yl)piperazin-1-yl)piperidine-1-carboxylate. To a stirred solution of 3-(5-bromo-4-fluoro-3-methyl-2-oxo-1,3-benzodiazol-1-yl)-1-[(4-methoxyphenyl)methyl]piperidine-2,6-dione (500 mg, 1.05 mmol, Intermediate UH) and tert-butyl (4R)-3,3-difluoro-4-(piperazin-1-yl)piperidine-1-carboxylate (320.56 mg, 1.050 mmol, Intermediate PN) in dioxane (5 mL) was added Cs₂CO₃ (684.06 mg, 2.100 mmol) in portions at rt. To the above mixture was added Pd-PEPPSI-IPentCl₂-methylpyridine (o-picoline) (88.30 mg, 0.105 mmol) in portions at rt. The resulting mixture was stirred for 2 h at 90° C. On completion, the mixture product was purified by reverse phase flash chromatography (WelFlash™ C18-I, 20-40 μm, 330 g; Eluent A: Water (plus 10 mmol/L FA or 10 mmol/L NH₄HCO₃); Eluent B: ACN; Gradient: 40%-80% B in 25 min; Flow rate: 80 mL/min; Detector: 220/254 nm; desired fractions were collected at 70% B and concentrated under reduced pressure) to afford the title compound (330 mg, 45% yield). LC/MS (ESI, m/z): [(M+1)]⁺=701.3.

Step 2—3-(5-(4-((R)-3,3-difluoropiperidin-4-yl)piperazin-1-yl)-4-fluoro-3-methyl-2-oxo-2,3-dihydro-1H-benzo[d]imidazol-1-yl)piperidine-2,6-dione. To a stirred solution of tert-butyl (4R)-3,3-difluoro-4-[4-(4-fluoro-1-{1-[(4-methoxyphenyl)methyl]-2,6-dioxopiperidin-3-yl}-3-methyl-2-oxo-1,3-benzodiazol-5-yl) piperazin-1-yl]piperidine-1-carboxylate (100 mg, 0.143 mmol) in TFA (5 mL) were added TfOH (0.5 mL) dropwise at rt. The resulting mixture was then stirred for 2 h at 70° C. On completion, the mixture was cooled to rt and concentrated under reduced pressure. The crude product was purified by reverse phase flash chromatography (WelFlash™ C18-I, 20-40 μm, 120 g; Eluent A: Water (plus 10 mmol/L NH₄HCO₃); Eluent B: ACN; Gradient: 25%-55% B in 25 min; Flow rate: 80 mL/min; Detector: 220/254 nm; desired fractions were collected at 34% B and concentrated under reduced pressure) to afford the title compound (140 mg, 60% yield) as a white solid. LC/MS (ESI, m/z): [(M+1)]⁺=481.2.

### Tert-butyl 2-[(5-bromopyrimidin-2-yl)methyl]-2,7-diazaspiro[3.5]nonane-7-carboxylate (Intermediate VA)

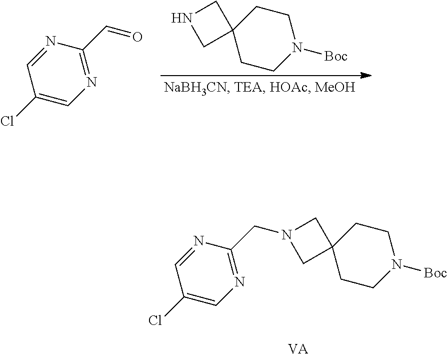 [SMILES: O=Cc1ncc(Cl)cn1 ; CC(C)(C)OC(=O)N1CCC2(CC1)CNC2 ; CC(C)(C)OC(=O)N1CCC2(CC1)CN(Cc1ncc(Cl)cn1)C2 ; "VA"]

To a stirred solution of tert-butyl 2,7-diazaspiro[3.5]nonane-7-carboxylate (605.15 mg, 2.674 mmol) in MeOH (10 mL) was added TEA (1.12 mL, 8.022 mmol) at rt. The resulting mixture was stirred for 5 min at rt. To the above mixture were added 5-bromopyrimidine-2-carbaldehyde (500 mg, 2.67 mmol, CAS #944902-05-0) and AcOH (0.77 mL, 13.4 mmol) at rt. The resulting mixture was stirred for an additional 20 min at rt. To the above mixture was added NaBH₃CN (504.07 mg, 8.022 mmol) at rt. The resulting mixture was stirred for an additional 2 h at 50° C. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 40%-60% B in 25 min; Flow rate: 80 mL/min; Detector: 220/254 nm; desired fractions were collected at 56% B and concentrated under reduced pressure) to afford the title compound (680 mg, 64% yield) as an off-white solid. LC/MS (ESI, m/z): [(M+H)]⁺=397.3, 399.3.

### 6-[4-(2-{2,7-diazaspiro[3.5]nonan-2-ylmethyl}pyrimidin-5-yl)-2,3-dihydroindol-1-yl]-N-[(1R,2S)-2-fluorocyclopropyl]-8-(methylamino)imidazo[1,2-b]pyridazine-3-carboxamide (Intermediate VB)

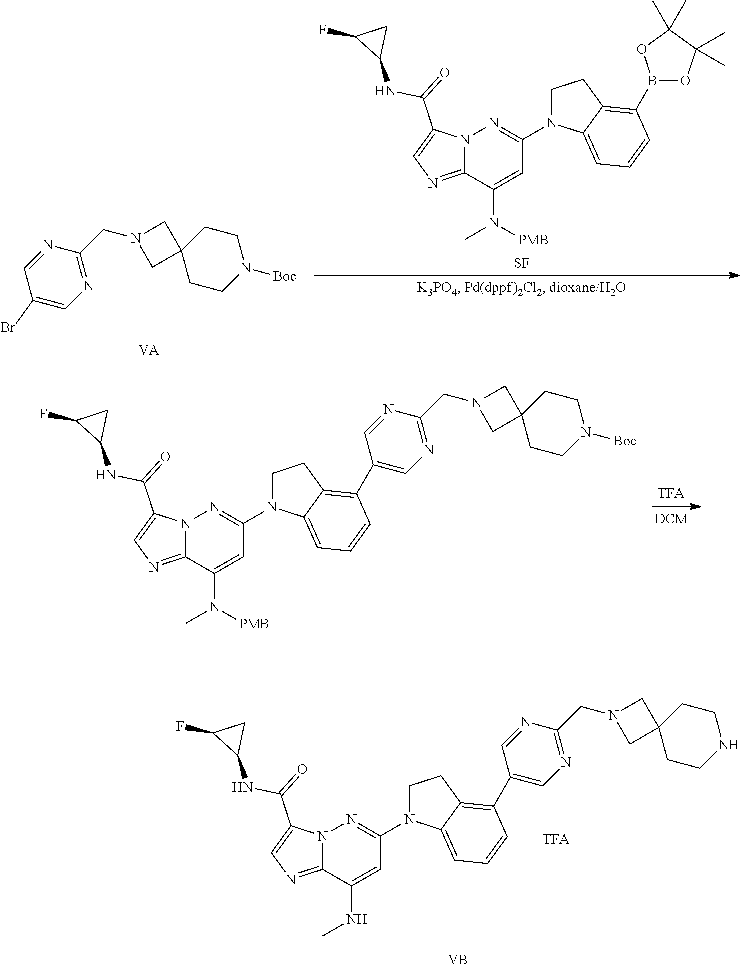

Step 1—Tert-butyl 2-({5-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyrimidin-2-yl}methyl)-2,7-diazaspiro[3.5]nonane-7-carboxylate. To a stirred mixture of tert-butyl 2-[(5-bromopyrimidin-2-yl)methyl]-2,7-diazaspiro[3.5]nonane-7-carboxylate (610 mg, 1.54 mmol, Intermediate VA) and N-[(1R,2S)-2-fluorocyclopropyl]-8-{[(4-methoxyphenyl)methyl](methyl)amino}-6-[4-(4,4,5,5-tetramethyl-1,3,2-dioxaborolan-2-yl)-2,3-dihydroindol-1-yl]imidazo[1,2-b]pyridazine-3-carboxamide (1034.42 mg, 1.689 mmol, Intermediate SF) in dioxane (10 mL) and H₂O (2 mL) were added K₃PO₄ (977.67 mg, 4.605 mmol) and Pd(dppf)Cl₂·CH₂Cl₂ (125.07 mg, 0.153 mmol) at rt. The resulting mixture was stirred for 2 h at 90° C. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L FA); Eluent B: ACN; Gradient: 70%-90% B in 25 min; Flow rate: 80 mL/min; Detector: 220/254 nm; desired fractions were collected at 83% B and concentrated under reduced pressure) to afford the title compound (440 mg, 36% yield) as a brown solid. LC/MS (ESI, m/z): [(M+H)]⁺=803.4.

Step 2—6-[4-(2-{2,7-diazaspiro[3.5]nonan-2-ylmethyl}pyrimidin-5-yl)-2,3-dihydroindol-1-yl]-N-[(1R,2S)-2-fluorocyclopropyl]-8-(methylamino)imidazo[1,2-b]pyridazine-3-carboxamide trifluoroacetate. To a stirred solution of tert-butyl 2-({5-[1-(3-{[(1R,2S)-2-fluorocyclopropyl]carbamoyl}-8-{1[(4-methoxyphenyl)methyl](methyl)amino}imidazo[1,2-b]pyridazin-6-yl)-2,3-dihydroindol-4-yl]pyrimidin-2-yl}methyl)-2,7-diazaspiro[3.5]nonane-7-carboxylate (440 mg, 0.548 mmol) in DCM (6 mL) was added TFA (2 mL) at rt. The resulting mixture was stirred for 1 h at rt. On completion, the mixture was concentrated under reduced pressure. The residue was purified by trituration with ethyl ether (30 mL) to give the title compound (300 mg) as a brown solid. LC/MS (ESI, m/z): [(M+H)]f=583.3.

### 6-(4-(5-((1,1-Difluoro-3,9-diazaspiro[5.5]undecan-3-yl)methyl)pyridin-2-yl)indolin-1-yl)-N-((1R,2R)-2-methoxycyclobutyl)-8-(methylamino)imidazo[1,2-b]pyridazine-3-carboxamide (Intermediate VC)

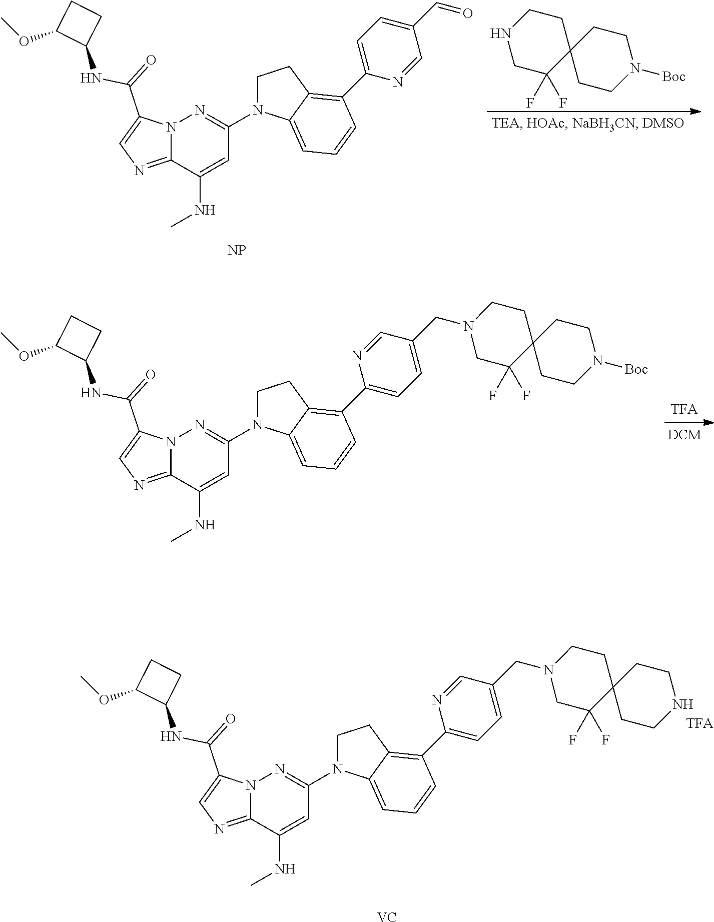

Step 1—Tert-butyl 7,7-difluoro-9-((6-(1-(3-(((1R,2R)-2-methoxycyclobutyl)carbamoyl)-8-(methylamino)imidazo[1,2-b]pyridazin-6-yl)indolin-4-yl)pyridin-3-yl)methyl)-3,9-diazaspiro[5.5]undecane-3-carboxylate. To a stirred mixture of tert-butyl 7,7-difluoro-3,9-diazaspiro[5.5]undecane-3-carboxylate (88 mg, 0.30 mmol) and Et₃N (61 mg, 0.60 mmol) in DMSO (6 mL) were added 6-(4-(5-formylpyridin-2-yl)indolin-1-yl)-N-((1R,2R)-2-methoxycyclobutyl)-8-(methylamino)imidazo[1,2-b]pyridazine-3-carboxamide (150 mg, 0.301 mmol, Intermediate NP) and HOAc (181 mg, 3.010 mmol) at rt. The resulting mixture was stirred for 1 h at 50° C. To the above mixture was added NaBH₃CN (95 mg, 1.505 mmol) in portions at rt. The resulting mixture was stirred for an additional 1 h at 50° C. On completion, the mixture was cooled to rt and purified directly by reversed-phase flash chromatography (Column: Spherical C18, 20˜40 μm, 330 g; Mobile Phase A: Water (plus 10 mM NH₄HCO₃), Mobile Phase B: ACN; Flow rate: 80 mL/min; Gradient (B %): 5%75%, 4 min; 75%˜90%, 20 min; 90%-95%; 2 min; 95%, 5 min; Detector: 254 nm; the fractions containing desired product were collected at 87% B and concentrated under reduced pressure) to afford the title compound (160 mg, 68% yield) as a white solid. LC/MS (ESI, m/z): [(M+1)]⁺=772.4.

Step 2—6-(4-(5-((1,1-Difluoro-3,9-diazaspiro[5.5]undecan-3-yl)methyl)pyridin-2-yl)indolin-1-yl)-N-((1R,2R)-2-methoxycyclobutyl)-8-(methylamino)imidazo[1,2-b]pyridazine-3-carboxamide trifluoroacetate. To a stirred mixture of tert-butyl 7,7-difluoro-9-((6-(1-(3-(((1R,2R)-2-methoxycyclobutyl)carbamoyl)-8-(methylamino)imidazo[1,2-b]pyridazin-6-yl)indolin-4-yl)pyridin-3-yl)methyl)-3,9-diazaspiro[5.5]undecane-3-carboxylate (160 mg, 0.207 mmol) in DCM (3 mL) was added TFA (1 mL) at rt. The resulting mixture was stirred for 1 h at rt. On completion, the resulting mixture was concentrated under reduced pressure to afford the title compound (130 mg, TFA salt) as a brown solid. LC/MS (ESI, m/z): [(M+1)]⁺=672.3.

### 3-(5-{4-[1-(3,3-Difluoropiperidin-4-yl)azetidin-3-yl]piperazin-1-yl}-4-fluoro-3-methyl-2-oxo-1,3-benzodiazol-1-yl)piperidine-2,6-dione (Intermediate VD)

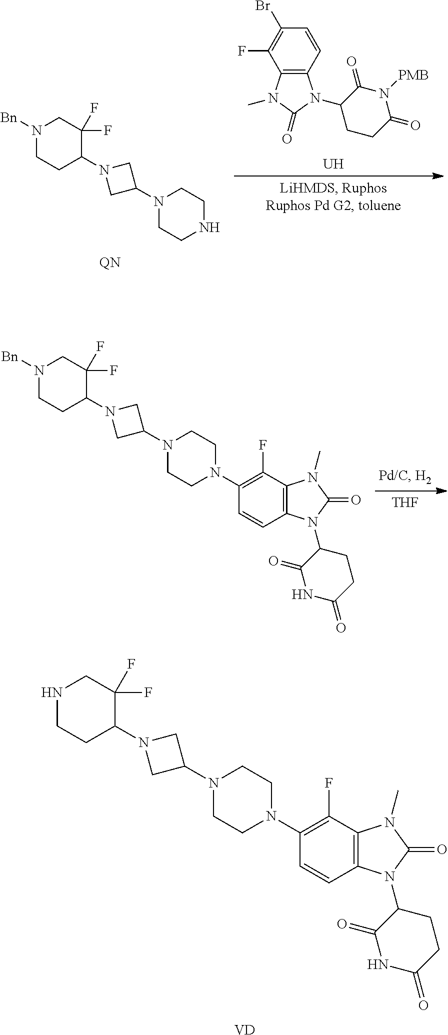

Step 1—3-(5-{4-[1-(1-Benzyl-3,3-difluoropiperidin-4-yl)azetidin-3-yl]piperazin-1-yl}-4-fluoro-3-methyl-2-oxo-1,3-benzodiazol-1-yl)piperidine-2,6-dione. To a stirred solution of 1-[1-(1-benzyl-3,3-difluoropiperidin-4-yl)azetidin-3-yl]piperazine (500 mg, 1.43 mmol, Intermediate QN), 3-(5-bromo-4-fluoro-3-methyl-2-oxo-1,3-benzodiazol-1-yl)piperidine-2,6-dione (508 mg, 1.43 mmol, Intermediate UH), RuPhos (133.15 mg, 0.285 mmol) and RuPhos-PdCl-2nd G (221.92 mg, 0.285 mmol) in toluene (10 mL) was added LiHMDS (8 mL, 47.810 mmol) dropwise at rt under nitrogen atmosphere. The resulting mixture was stirred for 2 h at 80° C. under nitrogen atmosphere. On completion, the mixture was cooled to rt and concentrated under reduced pressure. The mixture was purified by silica gel column chromatography (Mobile Phase A: PE, Mobile Phase B: EA; Gradient: 50% to 100% B in 25 min, 254 nm; the fractions containing the desired product were collected at 20% B and concentrated under reduced pressure) to afford the title compound (150 mg, 16% yield) as a yellow oil. LC/MS (ESI, m/z): [(M+1)]=626.3.

Step 2—3-(5-{4-[1-(3,3-Difluoropiperidin-4-yl)azetidin-3-yl]piperazin-1-yl}-4-fluoro-3-methyl-2-oxo-1,3-benzodiazol-1-yl)piperidine-2,6-dione. To a solution of 3-(5-{4-[1-(1-benzyl-3,3-difluoropiperidin-4-yl)azetidin-3-yl]piperazin-1-yl}-4-fluoro-3-methyl-2-oxo-1,3-benzodiazol-1-yl)piperidine-2,6-dione (150 mg, 0.080 mmol) in THF (5 mL) was added Pd/C (10 wt %, 42.52 mg) under nitrogen atmosphere. The reaction system was degassed under vacuum and purged with H₂ several times, then it was hydrogenated under H₂ balloon (˜1 atm) at 25° C. for 2 h. After completion of the reaction, Pd/C was filtered off through celite and the filter cake was washed with THF (3×10 mL). The corresponding filtrate was concentrated under reduced pressure to afford the title compound (30 mg, 70% yield) as a white oil. LC/MS (ESI, m/z): [(M+1)]⁺=536.4.

### Tert-butyl 4-(1-(3,3-difluoropiperidin-4-yl)azetidin-3-yl)piperazine-1-carboxylate (Intermediate VE)

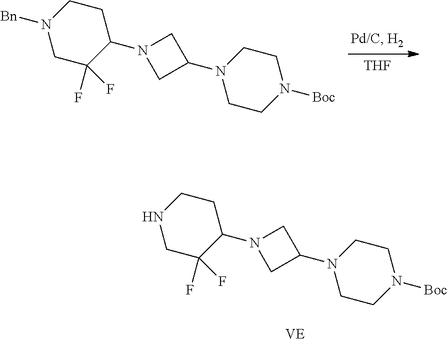 [SMILES: CC(C)(C)OC(=O)N1CCN(C2CN(C3CCN(Cc4ccccc4)CC3(F)F)C2)CC1 ; CC(C)(C)OC(=O)N1CCN(C2CN(C3CCNCC3(F)F)C2)CC1 ; "THF"]

To a solution of tert-butyl 4-[1-(1-benzyl-3,3-difluoropiperidin-4-yl)azetidin-3-yl]piperazine-1-carboxylate (700 mg, 1.55 mmol, synthesized via Steps 1-2 of Intermediate QN) in THF (20 mL) was added Pd/C (10 wt %, 826 mg) under nitrogen atmosphere. The reaction system was degassed under vacuum and purged with H₂ several times, then it was hydrogenated under H₂ balloon (˜1 atm) at 25° C. for 2 h. After completion of the reaction, Pd/C was filtered off through celite and the filter cake was washed with THF (3×60 mL). The corresponding filtrate was concentrated under reduced pressure to afford the title compound (500 mg, 89% tuekd) as a yellow solid. LC/MS (ESI, m/z): [(M+H)]⁺=361.2.

### 6-(4-((3,3-difluoro-4-(3-(piperazin-1-yl)azetidin-1-yl)piperidin-1-yl)methyl)indolin-1-yl)-N-((1R,2S)-2-fluorocyclopropyl)-8-(methylamino)imidazo[1,2-b]pyridazine-3-carboxamide (Intermediate VF)

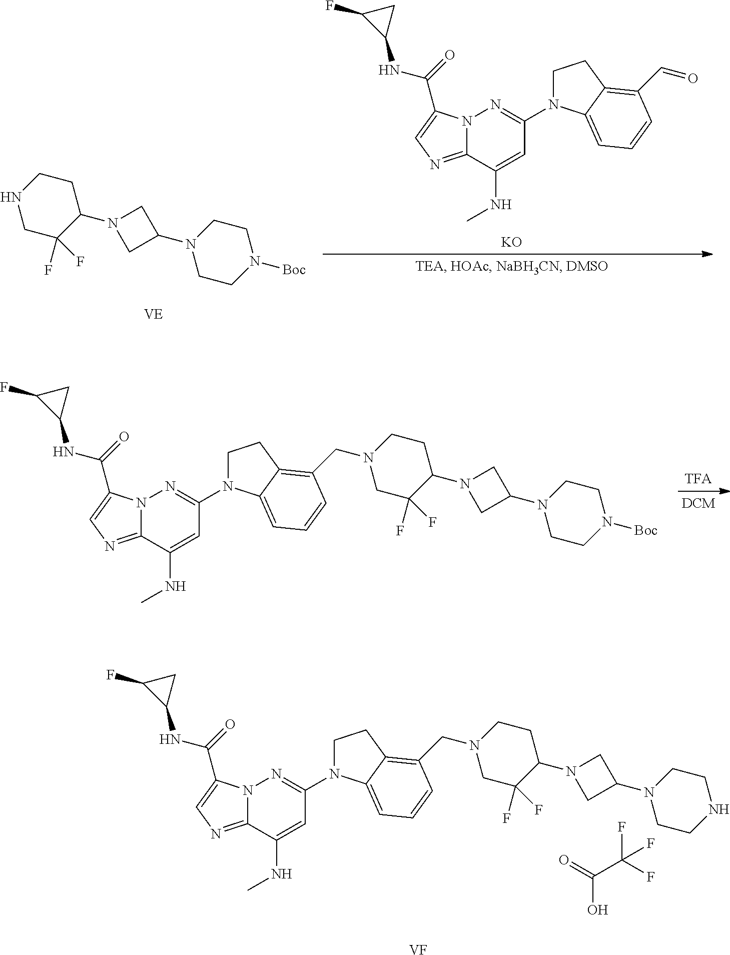

Step 1—Tert-butyl 4-(1-(3,3-difluoro-1-((1-(3-(((1R,2S)-2-fluorocyclopropyl)carbamoyl)-8-(methylamino)imidazo[1,2-b]pyridazin-6-yl)indolin-4-yl)methyl)piperidin-4-yl)azetidin-3-yl)piperazine-1-carboxylate. To a stirred solution of tert-butyl 4-(1-(3,3-difluoropiperidin-4-yl)azetidin-3-yl)piperazine-1-carboxylate (200 mg, 0.555 mmol, Intermediate VE) and TEA (112 mg, 1.110 mmol) in DMSO (5 mL) were added N-[(1R,2S)-2-fluorocyclopropyl]-6-(4-formyl-2,3-dihydroindol-1-yl)-8-(methylamino)imidazo[1,2-b]pyridazine-3-carboxamide (218 mg, 0.555 mmol, Intermediate KO) and HOAc (99 mg, 1.67 mmol) in turns at rt under nitrogen atmosphere. To the above mixture was added NaBH₃CN (104.60 mg, 1.665 mmol) in portions over 2 min at rt. The resulting mixture was stirred for an additional 2 h at 50° C. After completion of the reaction, the reaction mixture was cooled to rt and purified directly by reverse phase flash chromatography (Column: WelFlash™ C18-I, 20-40 μm, 120 g; Eluent A: Water (10 mmol/L NH₄HCO₃); Eluent B: ACN; Gradient: 25%-55% B in 25 min; Flow rate: 60 mL/min; Detector: 220/254 nm; desired fractions were collected at 47% B and concentrated under reduced pressure) to afford the title compound (180 mg, 43% yield) as a yellow oil. LC/MS (ESI, m/z): [(M+H)]⁺=739.4.

Step 2—6-(4-((3,3-Difluoro-4-(3-(piperazin-1-yl)azetidin-1-yl)piperidin-1-yl)methyl)indolin-1-yl)-N-((1R,2S)-2-fluorocyclopropyl)-8-(methylamino)imidazo[1,2-b]pyridazine-3-carboxamide trifluoroacetate. To a stirred solution of tert-butyl 4-[1-(3,3-difluoro-1-{[L-(3-{[(1R,2S)-2-fluorocyclopropyl]carbamoyl}-8-(methylamino)imidazo[1,2-b]pyridazin-6-yl)-2,3-dihydroindol-4-yl]methyl}piperidin-4-yl)azetidin-3-yl]piperazine-1-carboxylate (180 mg, 0.244 mmol) in DCM (6 mL) was added TFA (2 mL) dropwise at rt under nitrogen atmosphere. The resulting mixture was stirred for 1 h at rt. On completion, the mixture was purified by trituration with Et₂O (10 mL) to afford the title compound (150 mg, 96% yield) as a white solid. LC/MS (ESI, m/z): [(M+H)]⁺=639.3.

### 5-(2,4-Dioxo-1,3-diazinan-1-yl)-2-fluoro-4-methylbenzoic acid (Intermediate VG)

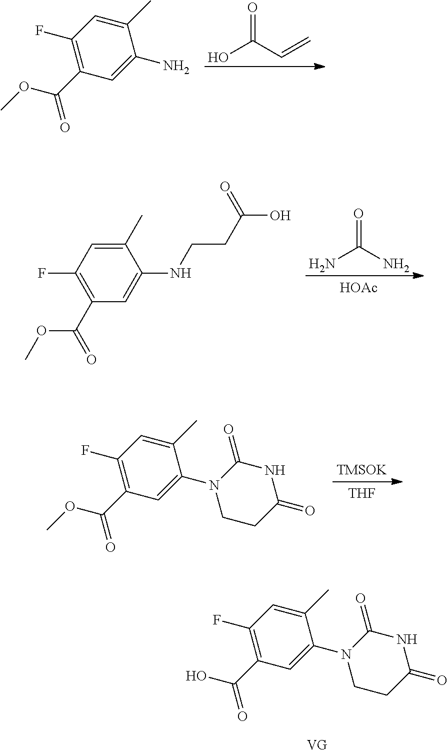

Step 1—3-((4-Fluoro-5-(methoxycarbonyl)-2-methylphenyl)amino)propanoic acid. To a stirred solution of methyl 5-amino-2-fluoro-4-methylbenzoate (5.00 g, 27.3 mmol) was added acrylic acid (3.93 g, 54.6 mmol) dropwise at rt under nitrogen atmosphere. The resulting mixture was stirred for 16 h at 100° C. under nitrogen atmosphere. On completion, the mixture was cooled to rt and concentrated under reduced pressure. The resulting mixture was diluted with water (500 mL) and was extracted with EtOAc (2×300 mL). The combined organic layers were washed with brine (500 mL), and dried over anhydrous Na₂SO₄. After filtration, the filtrate was concentrated under reduced pressure to give the title compound (3.50 g, 50% yield) as a brown solid. LC/MS (ESI, m/z): [(M+1)]⁺=256.0.

Step 2—Methyl 5-(2,4-dioxo-1,3-diazinan-1-yl)-2-fluoro-4-methylbenzoate. To a stirred solution of 3-{[4-fluoro-5-(methoxycarbonyl)-2-methylphenyl]amino}propanoic acid (3.5 g, 13.71 mmol) in HOAc (5 mL) was added urea (0.82 g, 13.7 mmol) in portions at rt under nitrogen atmosphere. The resulting mixture was stirred for 16 h at 110° C. under nitrogen atmosphere. On completion, the mixture was cooled and concentrated under reduced pressure. The residue was purified by reverse phase flash chromatography (Column: WelFlash™ C18-I, 20-40 μm, 120 g; Eluent A: Water (plus 10 mmol/L FA); Eluent B: ACN; Gradient: 25%-55% B in 25 min; Flow rate: 60 mL/min; Detector: 220/254 nm; desired fractions were collected at 49% B and concentrated under reduced pressure) to afford the title compound (2.00 g, 52% yield) as a yellow oil. LC/MS (ESI, m/z): [(M+1)]⁺=281.1.

Step 3—5-(2,4-Dioxo-1,3-diazinan-1-yl)-2-fluoro-4-methylbenzoic acid. To a stirred solution of methyl 5-(2,4-dioxo-1,3-diazinan-1-yl)-2-fluoro-4-methylbenzoate (500 mg, 1.78 mmol) in THF (20 mL) was added potassium trifluoroacetate (1.14 g, 8.92 mmol) dropwise at rt under nitrogen atmosphere. The resulting mixture was stirred for 1 h at rt under nitrogen atmosphere. On completion, the mixture was concentrated under reduced pressure. The residue was purified by reverse phase flash chromatography (Column: WelFlash™ C18-I, 20-40 μm, 330 g; Eluent A: Water (plus 10 mmol/L FA); Eluent B: ACN; Gradient: 25%-55% B in 25 min; Flow rate: 80 mL/min; Detector: 220/254 nm; desired fractions were collected at 50% B and concentrated under reduced pressure) to afford the title compound (300 mg, 63% yield) as a white solid. LC/MS (ESI, m/z): [(M+1)]=267.1.

### (1S,3S)-3-fluorocyclobutan-1-amine hydrochloride (CAS #1408075-13-7) (Intermediate VH)

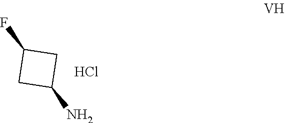

### (1R,2R)-2-fluorocyclobutan-1-amine hydrochloride (CAS #2920233-59-4) (Intermediate VI)

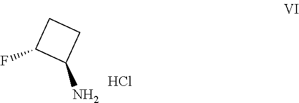

### 6-(4-(5-((7-(4-chloro-3-(2,4-dioxotetrahydropyrimidin-1(2H)-yl)benzoyl)-5,5-difluoro-2,7-diazaspiro[3.5]nonan-2-yl)methyl)pyridin-2-yl)indolin-1-yl)-8-(methylamino)imidazo[1,2-b]pyridazine-3-carboxylic acid (Intermediate VJ)

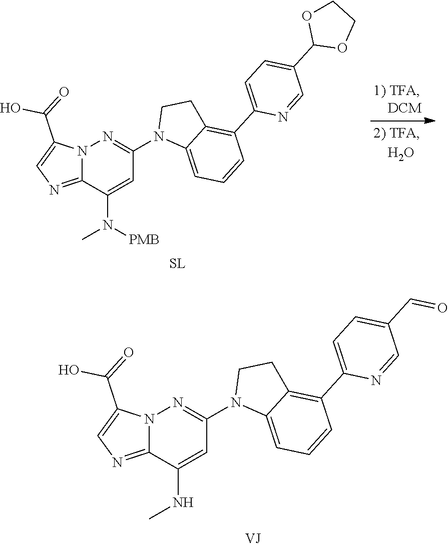

Step 1—6-[4-(5-Formylpyridin-2-yl)-2,3-dihydroindol-1-yl]-8-(methylamino)imidazo[1,2-b]pyridazine-3-carboxylic acid. To a stirred mixture of 6-{4-[5-(1,3-dioxolan-2-yl)pyridin-2-yl]-2,3-dihydroindol-1-yl}-8-{[(4-methoxyphenyl)methyl](methyl)amino}imidazo[1,2-b]pyridazine-3-carboxylic acid (2.20 g, 3.80 mmol, Intermediate SL) in DCM (40 mL) was added TFA (40 mL) at rt. The resulting mixture was stirred for 1 h at rt. Next, the mixture was concentrated under reduced pressure. The residue was dissolved in H₂O (40 mL) and TFA (40 mL). The resulting mixture was stirred for an additional 1 h at 50° C. On completion, the mixture was cooled to rt and was acidified to pH 3 with NaOH (aq). The precipitated solids were collected by filtration and washed with water (3×20 mL) to give the title compound (1.6 g) as a yellow solid. LC/MS (ESI, m/z): [(M+1)]⁺=415.1.

### 6-(4-(5-((7-(4-chloro-3-(2,4-dioxotetrahydropyrimidin-1(2H)-yl)benzoyl)-5,5-difluoro-2,7-diazaspiro[3.5]nonan-2-yl)methyl)pyridin-2-yl)indolin-1-yl)-8-(methylamino)imidazo[1,2-b]pyridazine-3-carboxylic acid (Intermediate VK)

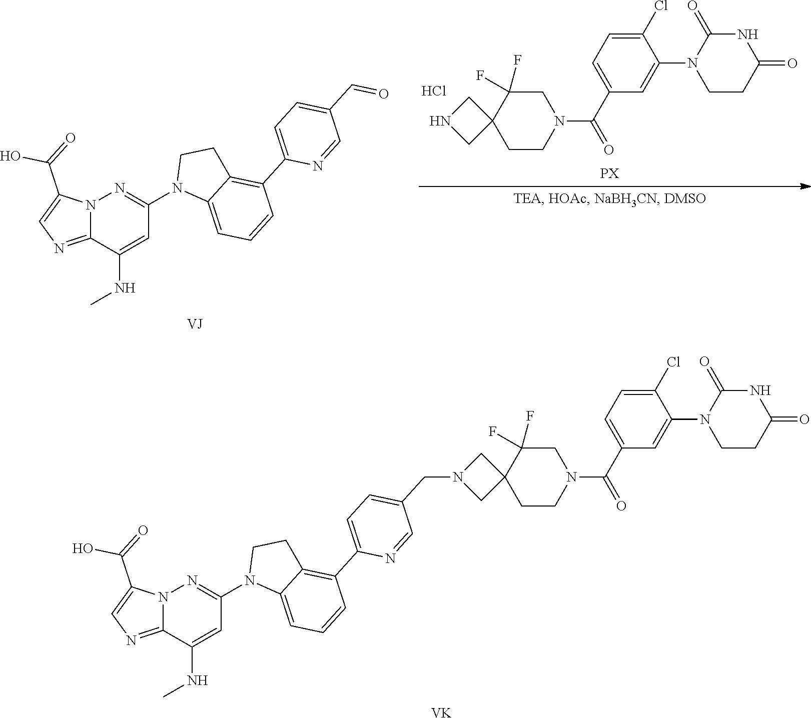 [SMILES: CNc1cc(N2CCc3c(-c4ccc(C=O)cn4)cccc32)nn2c(C(=O)O)cnc12 ; Cl ; O=C1CCN(c2cc(C(=O)N3CCC4(CNC4)C(F)(F)C3)ccc2Cl)C(=O)N1 ; CNc1cc(N2CCc3c(-c4ccc(CN5CC6(CCN(C(=O)c7ccc(Cl)c(N8CCC(=O)NC8=O)c7)CC6(F)F)C5)cn4)cccc32)nn2c(C(=O)O)cnc12 ; "VK"]

Step 1—6-(4-(5-((7-(4-Chloro-3-(2,4-dioxotetrahydropyrimidin-1(2H)-yl)benzoyl)-5,5-difluoro-2,7-diazaspiro[3.5]nonan-2-yl)methyl)pyridin-2-yl)indolin-1-yl)-8-(methylamino)imidazo[1,2-b]pyridazine-3-carboxylic acid. To a stirred solution of 1-(2-chloro-5-(5,5-difluoro-2,7-diazaspiro[3.5]nonane-7-carbonyl)phenyl)dihydropyrimidine-2,4(1H,3H)-dione trifluoroacetate (99 mg, 0.24 mmol, Intermediate PX) and TEA (24 mg, 0.241 mmol) in DMSO (3 mL) were added 6-(4-(5-formylpyridin-2-yl)indolin-1-yl)-8-(methylamino)imidazo[1,2-b]pyridazine-3-carboxylic acid (100 mg, 0.241 mmol, Intermediate VJ) and HOAc (43 mg, 0.723 mmol) at rt. The resulting mixture was stirred for 2 h at 50° C. Then NaBH₃CN (60 mg, 0.964 mmol) was added in portions at rt. The resulting mixture was stirred overnight at 50° C. On completion, the mixture was cooled to rt and purified directly by reverse phase flash chromatography (Column: WelFlash™ C18-I, 20-40 μm, 120 g; Eluent A: Water (plus 10 mmol/L NH₄CO₃); Eluent B: ACN; Gradient: 20%-50% B in 30 min; Flow rate: 60 mL/min; Detector: 220/254 nm; desired fractions were collected at 48% B and concentrated under reduced pressure) to afford the title compound (140 mg, 71% yield) as a light yellow solid. LC/MS (ESI, m/z): [(M+1)]⁺=811.3.

### (1r,3r)-3-fluorocyclobutan-1-amine (CAS #1260670-33-4) (Intermediate VL)

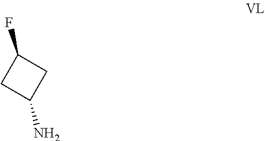

### 6-(4-(3-(3,3-difluoro-4-(3,9-diazaspiro[5.5]undecan-3-yl)piperidin-1-yl)prop-1-yn-1-yl)indolin-1-yl)-N-((1R,2S)-2-fluorocyclopropyl)-8-(methylamino)imidazo[1,2-b]pyridazine-3-carboxamide (Intermediate VM)

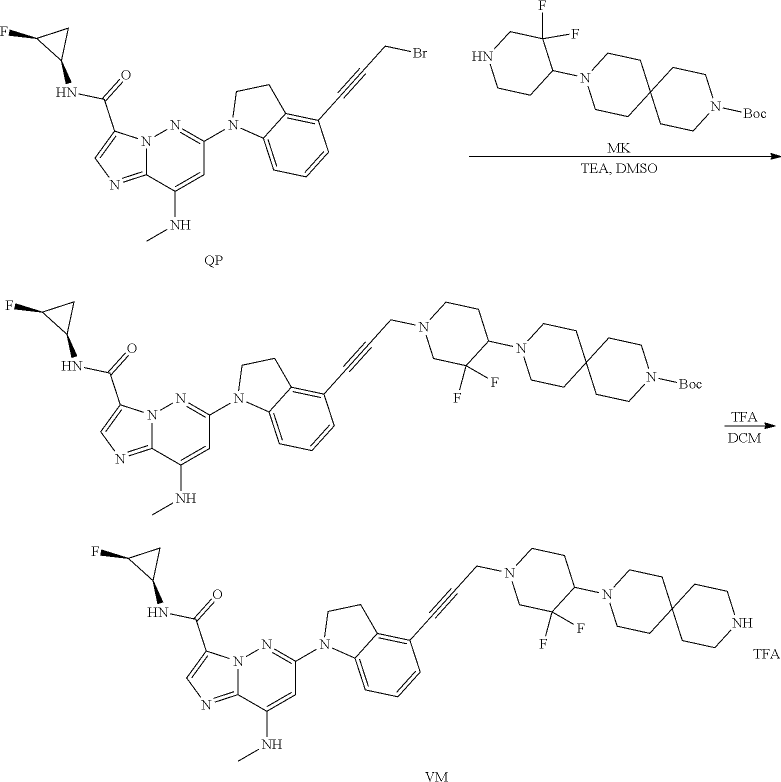

Step 1—Tert-butyl 9-(3,3-difluoro-1-(3-(1-(3-(((1R,2S)-2-fluorocyclopropyl)carbamoyl)-8-(methylamino)imidazo[1,2-b]pyridazin-6-yl)indolin-4-yl)prop-2-yn-1-yl)piperidin-4-yl)-3,9-diazaspiro[5.5]undecane-3-carboxylate. To a stirred solution of tert-butyl 9-(3,3-difluoropiperidin-4-yl)-3,9-diazaspiro[5.5]undecane-3-carboxylate (40 mg 0.11 mmol, Intermediate MK) and 6-[4-(3-bromoprop-1-yn-1-yl)-2,3-dihydroindol-1-yl]-N-[(1R,2S)-2-fluorocyclopropyl]-8-(methylamino)imidazo[1,2-b]pyridazine-3-carboxamide (52 mg, 0.11 mmol, Intermediate QP) in NMP (1 mL) was added TEA (32 mg, 0.316 mmol) at rt. The resulting mixture was stirred for 1 h at 80° C. On completion, the mixture was cooled to rt and purified directly by reverse phase flash chromatography (Column: WelFlash™ C18-I, 20-40 μm, 120 g; Eluent A: Water (plus 10 mmol/L FA); Eluent B: ACN; Gradient: 35%-65% B in 25 min; Flow rate: 60 mL/min; Detector: 220/254 nm; desired fractions were collected at 52% B and concentrated under reduced pressure) to afford the title compound (55 mg, 66% yield) as a light yellow solid. LC/MS (ESI, m/z): [(M+1)]⁺=776.4.

Step 2—6-(4-(3-(3,3-Difluoro-4-(3,9-diazaspiro[5.5]undecan-3-yl)piperidin-1-yl)prop-1-yn-1-yl)indolin-1-yl)-N-((1R,2S)-2-fluorocyclopropyl)-8-(methylamino)imidazo[1,2-b]pyridazine-3-carboxamide trifluoroacetate. To a stirred solution of tert-butyl 9-(3,3-difluoro-1-{3-[1-(3-{[(1R,2S)-2-fluorocyclopropyl]carbamoyl}-8-(methylamino)imidazo[1,2-b]pyridazin-6-yl)-2,3-dihydroindol-4-yl]prop-2-yn-1-yl}piperidin-4-yl)-3,9-diazaspiro[5.5]undecane-3-carboxylate (55 mg, 0.071 mmol) in DCM (1 mL) was added TFA (0.3 mL) at rt. The resulting mixture was stirred for 1 h at rt. On completion, the resulting mixture was concentrated under reduced pressure. The residue was purified by trituration with Et₂O (2 mL) to afford the title compound (50 mg, TFA salt) as a brown solid. LC/MS (ESI, m/z): [(M+1)]⁺=676.3.

### 2-Chloro-5-(2,4-dioxo-1,3-diazinan-1-yl)-4-methylbenzoic acid (Intermediate VN)

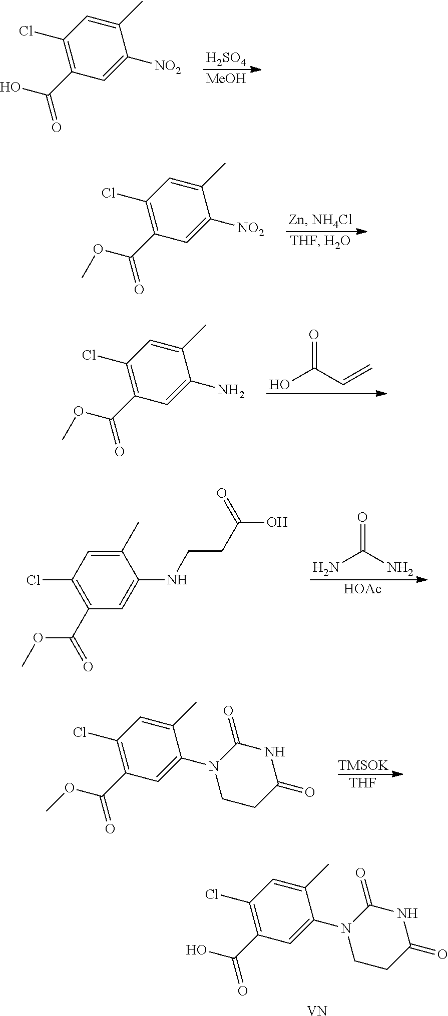

Step 1—Methyl 2-chloro-4-methyl-5-nitrobenzoate. To a stirred solution of 2-chloro-4-methyl-5-nitrobenzoic acid (3 g, 14 mmol) in MeOH (20 mL) was added H₂SO₄ (0.74 mL, 14 mmol) dropwise at rt. The resulting mixture was stirred for 1 h at 70° C. On completion, the mixture was cooled to rt and basified to pH 8 with saturated NaOH (aq.) and extracted with EtOAc (3×100 mL). The combined organic layers were washed with brine (1×100 mL) and dried over anhydrous Na₂SO₄. After filtration, the filtrate was concentrated under reduced pressure. The residue was purified by silica gel column chromatography, eluted with PE/EA (5:1-1:3) to afford the title compound (1.7 g, 53% yield) as a white solid. LC/MS (ESI, m/z): [(M+H)]⁺=230.0.

Step 2—Methyl 5-amino-2-chloro-4-methylbenzoate. To a stirred mixture of methyl 2-chloro-4-methyl-5-nitrobenzoate (1.65 g, 7.19 mmol) in THF (20 mL) were added NH4Cl (1.92 g, 35.9 mmol) and H₂O (10 mL) at rt. To the above mixture was added Zn (2.35 g, 35.9 mmol) at rt. The resulting mixture was stirred for 1 h at rt. On completion, the mixture was filtered, and the filter cake was washed with EtOAc (3×10 mL). The filtrate was concentrated under reduced pressure. The residue was purified by silica gel column chromatography, eluted with PE/EA (5:1-1:3), to afford the title compound (1.22 g, 85% yield) as a yellow oil. LC/MS (ESI, m/z): [(M+H)]⁺=200.0.

Step 3—3-{[4-Chloro-5-(methoxycarbonyl)-2-methylphenyl]amino}propanoic acid. Into an 8 mL vial were added methyl 5-amino-2-chloro-4-methylbenzoate (1.17 g, 5.86 mmol) and acrylic acid (1.27 g, 17.6 mmol) at rt. The resulting mixture was stirred for 1 h at 100° C. On completion, the mixture was cooled to rt and diluted with water (50 mL), then extracted with EtOAc (3×100 mL). The combined organic layers were washed with brine (1×100 mL), and dried over anhydrous Na₂SO₄. After filtration, the filtrate was concentrated under reduced pressure to give the title compound (2.2 g) as a yellow solid. LC/MS (ESI, m/z): [(M+H)]⁺=272.1.

Step 4—Methyl 2-chloro-5-(2,4-dioxo-1,3-diazinan-1-yl)-4-methylbenzoate. To a stirred mixture of 3-{[4-chloro-5-(methoxycarbonyl)-2-methylphenyl]amino}propanoic acid (2.1 g, 7.7 mmol) in HOAc (25 mL) was added urea (3.25 g, 54.1 mmol) at rt. The resulting mixture was stirred overnight at 110° C. On completion, the mixture was cooled to rt and diluted with water (100 mL). The resulting mixture was extracted with EtOAc (3×300 mL). The combined organic layers were washed with brine (1×200 mL), and dried over anhydrous Na₂SO₄. After filtration, the filtrate was concentrated under reduced pressure. The residue was purified by silica gel column chromatography, eluting with DCM/EA (5:1-1:3), afford the title compound (1 g, 44% yield) as a yellow solid. LC/MS (ESI, m/z): [(M+H)]⁺=297.1.

Step 5—2-Chloro-5-(2,4-dioxo-1,3-diazinan-1-yl)-4-methylbenzoic acid. To a stirred mixture of methyl 2-chloro-5-(2,4-dioxo-1,3-diazinan-1-yl)-4-methylbenzoate (1 g, 3 mmol) in THF (10 mL) was added potassium trimethylsilanolate (1.30 g, 10.1 mmol) at rt. The resulting mixture was stirred for 1 h at rt. On completion, the mixture was acidified to pH 4 with FA. The precipitated solids were collected by filtration and washed with THF (2×10 mL). The solids were purified by reverse phase flash chromatography (Column: WelFlash™ C18-1, 20-40 μm, 120 g; Eluent A: Water (plus 10 mmol/L FA); Eluent B: ACN; Gradient: 10%-30% B in 25 min; Flow rate: 60 mL/min; Detector: 220/254 nm; desired fractions were collected at 21% B and concentrated under reduced pressure) to afford the title compound (700 mg, 74% yield) as a white solid. LC/MS (ESI, m/z): [(M+H)]⁺=283.1.

### Tert-butyl 9-{[6-(2,3-dihydro-1H-indol-4-yl)pyridin-3-yl]methyl}-3-azaspiro[5.5]undecane-3-carboxylate (Intermediate VO)

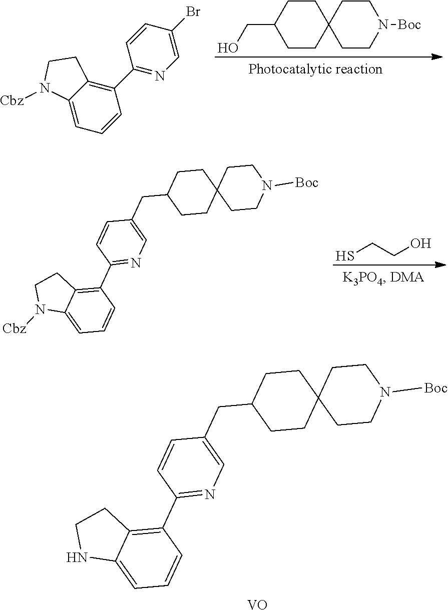

Step 1—Tert-butyl 9-[(6-{1-[(benzyloxy)carbonyl]-2,3-dihydroindol-4-yl}pyridin-3-yl)methyl]-3-azaspiro[5.5]undecane-3-carboxylate. To a stirred mixture of tert-butyl 9-(hydroxymethyl)-3-azaspiro[5.5]undecane-3-carboxylate (519.34 mg, 1.833 mmol, CAS #1341036-19-8) in MTBE (5 mL) was added 5,7-di-tert-butyl-3-phenylbenzo[d]oxazol-3-ium tetrafluoroborate (774.54 mg, 1.955 mmol) at rt under nitrogen atmosphere. The resulting mixture was stirred for 5 min at rt under nitrogen atmosphere. To the above mixture was added pyridine (154.61 mg, 1.955 mmol) at rt under nitrogen atmosphere and the mixture was stirred for 5 min at rt. The resulting mixture was filtered, the filter cake was washed with MTBE (1 mL). The filtrate was added to a mixture of benzyl 4-(5-bromopyridin-2-yl)-2,3-dihydroindole-1-carboxylate (500 mg, 1.222 mmol, Step 1 of Intermediate PT), IR[DF(CF₃)PPY]₂(DTBPY)PF₆ (68.53 mg, 0.061 mmol), 8,8-dibromo-4,12-di-tert-butyl-7$l\hat{ }{4},9l\hat{ }{4}-diaza-8-nickelatricyclo[7.4.0.0\hat{ }{2,7}]trideca-1(13),2 (29.74 mg, 0.061 mmol), 1-azabicyclo[2.2.2]octane (237.70 mg, 2.139 mmol) and 2,3-dihydro-1H-isoindole-1,3-dione (35.95 mg, 0.244 mmol) in DMA (10 mL) at rt under nitrogen atmosphere. The resulting mixture was stirred for 10 min at rt under nitrogen atmosphere. The vial was stirred at 1500 rpm stir rate and irradiated under 450 nm LED modules at 100% light intensity with maxed fan speed of 3500 rpm stirring rate in a PennOC Integrated Photoreactor for 16 hrs. On completion, the mixture was concentrated under vacuum. The residue was purified by reverse phase flash chromatography (Column: WelFlash™ C18-I, 20-40 μm, 330 g; Eluent A: Water (plus 10 mmol/L FA); Eluent B: ACN; Gradient: 65%-100% B in 25 min; Flow rate: 80 mL/min; Detector: 220/254 nm; desired fractions were collected at 100% B and concentrated under reduced pressure) to afford the title compound (450 mg, 62% yield) as a brown solid. LC/MS (ESI, m/z): [(M+H)]⁺=596.3.

Step 2—Tert-butyl 9-{[6-(2,3-dihydro-1H-indol-4-yl)pyridin-3-yl]methyl}-3-azaspiro[5.5]undecane-3-carboxylate. To a stirred mixture of tert-butyl 9-[(6-{1-[(benzyloxy)carbonyl]-2,3-dihydroindol-4-yl}pyridin-3-yl)methyl]-3-azaspiro[5.5]undecane-3-carboxylate (450 mg, 0.755 mmol) and #-mercaptoethanol (118.02 mg, 1.510 mmol) in DMA (10 mL) was added K₃PO₄ (641.30 mg, 3.020 mmol) at rt under nitrogen atmosphere. The resulting mixture was stirred for 16 h at 75° C. under nitrogen atmosphere. On completion, the mixture was cooled to rt and purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 65%-100% B in 25 min; Flow rate: 80 mL/min; Detector: 220/254 nm; desired fractions were collected at 100% B and concentrated under reduced pressure) to afford the title compound (320 mg, 92% yield) as a brown oil. LC/MS (ESI, m/z): [(M+H)]⁺=462.3.

### 6-[4-(5-{3-Azaspiro[5.5]undecan-9-ylmethyl}pyridin-2-yl)-2,3-dihydroindol-1-yl]-N-[(1R,2S)-2-fluorocyclopropyl]-8-(methylamino)imidazo[1,2-b]pyridazine-3-carboxamide (Intermediate VP)

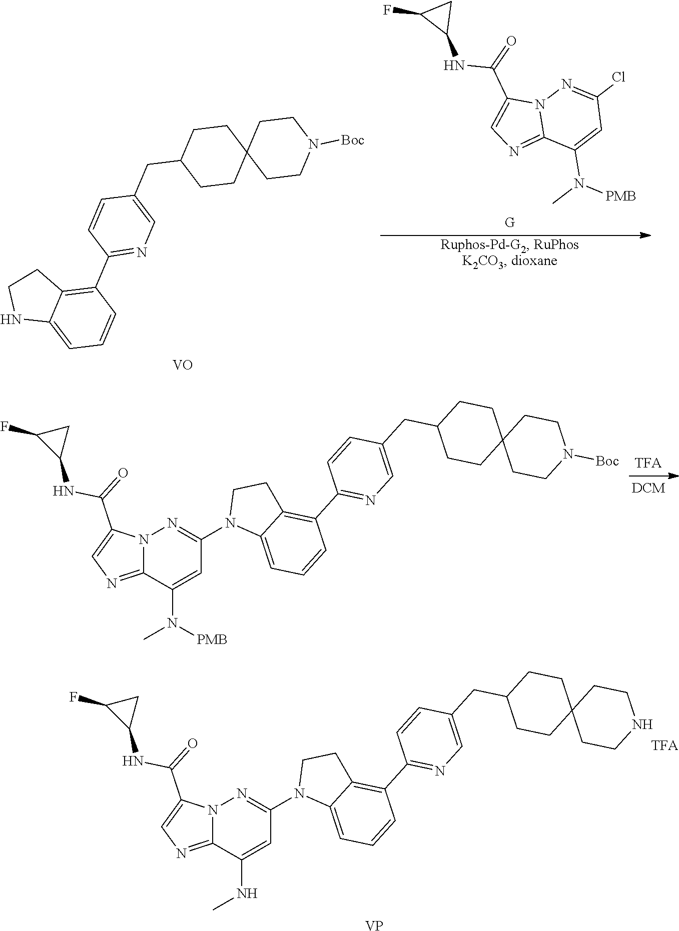

Step 1—Tert-butyl 9-({6-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyridin-3-yl}methyl)-3-azaspiro[5.5]undecane-3-carboxylate. To a stirred mixture of tert-butyl 9-{[6-(2,3-dihydro-1H-indol-4-yl)pyridin-3-yl]methyl}-3-azaspiro[5.5]undecane-3-carboxylate (320 mg, 0.693 mmol, Intermediate VO), 6-chloro-N-[(1R,2S)-2-fluorocyclopropyl]-8-{[(4-methoxyphenyl)methyl](methyl)amino}imidazo[1,2-b]pyridazine-3-carboxamide (335.91 mg, 0.832 mmol, Intermediate G) and K₂CO₃ (287.40 mg, 2.079 mmol) in 1,4-dioxane (10 mL) were added RuPhos (64.69 mg, 0.139 mmol) and RuPhos-PdCl-2nd G (64.69 mg, 0.139 mmol) at rt under nitrogen atmosphere. The resulting mixture was stirred for 1 h at 90° C. under nitrogen atmosphere. On completion, the mixture was cooled to rt and concentrated under vacuum. The residue was purified by reverse phase flash chromatography (Column: WelFlash™ C18-I, 20-40 μm, 330 g; Eluent A: Water (plus 10 mmol/L FA); Eluent B: ACN; Gradient: 65%-100% B in 25 min; Flow rate: 80 mL/min; Detector: 220/254 un; desired fractions were collected at 100% B and concentrated under reduced pressure) to afford the title compound (349 mg, 61% yield) as a brown solid. LC/MS (ESI, m/z): [(M+H)]⁺=829.0.

Step 2—6-[4-(5-{3-Azaspiro[5.5]undecan-9-ylmethyl}pyridin-2-yl)-2,3-dihydroindol-1-yl]-N-[(1R,2S)-2-fluorocyclopropyl]-8-(methylamino)imidazo[1,2-b]pyridazine-3-carboxamide trifluoroacetate. To a stirred solution of tert-butyl 9-({6-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyridin-3-yl}methyl)-3-azaspiro[5.5]undecane-3-carboxylate (349 mg, 0.421 mmol) in DCM (4 mL) was added TFA (4 mL) dropwise at rt under nitrogen atmosphere. The resulting mixture was stirred for 1 h at rt under nitrogen atmosphere. On completion, the mixture was concentrated under vacuum. The residue was purified by trituration with Et₂O (40 mL) to afford the title compound (289 mg, 97% yield) as a yellow solid. LC/MS (ESI, m/z): [(M+H)]⁺=609.3.

### 6-Chloro-4-(4,4,5,5-tetramethyl-1,3,2-dioxaborolan-2-yl)indoline (Intermediate VQ)

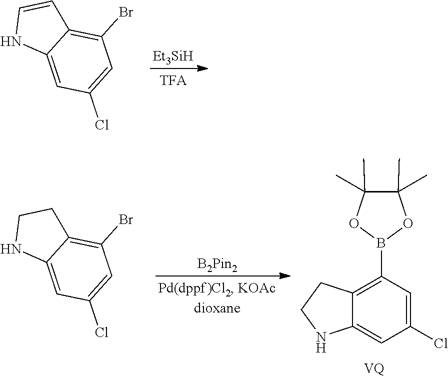

Step 1—4-Bromo-6-chloroindoline. To a stirred solution of 4-bromo-6-chloro-1H-indole (1.00 g, 4.33 mmol) in TFA (15 mL) was added Et₃SiH (1.51 g, 13.01 mmol) in portions at rt under nitrogen atmosphere. The resulting mixture was stirred for 1 h at rt under nitrogen atmosphere. The resulting mixture was concentrated under vacuum. The residue was purified by silica gel column chromatography, eluted with PE/EA (5:1), to afford the title compound (900 mg, 89% yield) as a yellow oil. LC/MS (ESI, m/z): [(M+1)]⁺=232.0, 234.0.

Step 2—6-Chloro-4-(4,4,5,5-tetramethyl-1,3,2-dioxaborolan-2-yl)indoline. To a stirred solution of 4-bromo-6-chloroindoline (900 mg, 3.87 mmol) and 4,4,5,5-tetramethyl-2-(4,4,5,5-tetramethyl-1,3,2-dioxaborolan-2-yl)-1,3,2-dioxaborolane (1.40 g, 5.80 mmol) in dioxane (10 mL) were added Pd(dppf)Cl₂ (283 mg, 0.382 mmol) and KOAc (1.14 g, 11.6 mmol) in turns at rt under nitrogen atmosphere. The resulting mixture was stirred then for 1 h at 90° C. under nitrogen atmosphere. On completion, the mixture was cooled to rt and concentrated under reduced pressure. The residue was purified by silica gel column chromatography, eluted with PE/EA (5:1), to afford the title compound (1.00 g, 92% yield) as a yellow oil. LC/MS (ESI, m/z): [(M+1)]⁺=280.1.

### 4-(5-(1,3-Dioxolan-2-yl)pyridin-2-yl)-6-chloroindoline (Intermediate VR)

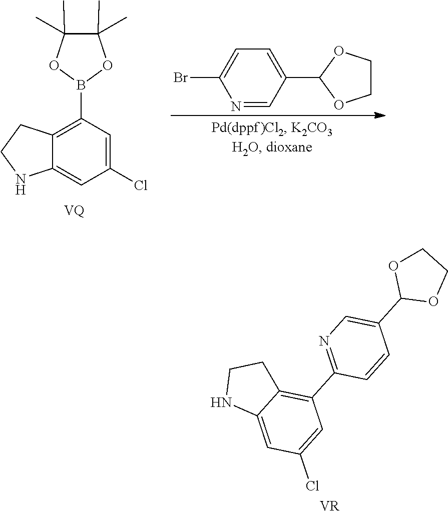

To a stirred solution of 6-chloro-4-(4,4,5,5-tetramethyl-1,3,2-dioxaborolan-2-yl)indoline (1.00 g, 3.57 mmol. Intermediate VQ) and 2-bromo-5-(1,3-dioxolan-2-yl)pyridine (0.91 g, 3.93 mmol) in dioxane (15 mL) and H₂O (3 mL) were added Pd(dppf)Cl₂CH₂Cl₂ (0.29 g, 0.35 mmol) and K₂CO₃ (1.48 g, 10.73 mmol) in turns at rt under nitrogen atmosphere. The resulting mixture was stirred for 1 h at 90° C. under nitrogen atmosphere. On completion, the mixture was cooled to rt and concentrated under reduced pressure. The residue was purified by silica gel column chromatography, eluted with PE/EA (1:1), to afford the title compound (510 mg, 47% yield) as a yellow oil. LC/MS (ESI, m/z): [(M+1)]=303.1.

### 6-(6-chloro-4-(5-formylpyridin-2-yl)indolin-1-yl)-N-((1R,2S)-2-fluorocyclopropyl)-8-(methylamino)imidazo[1,2-b]pyridazine-3-carboxamide (Intermediate VS)

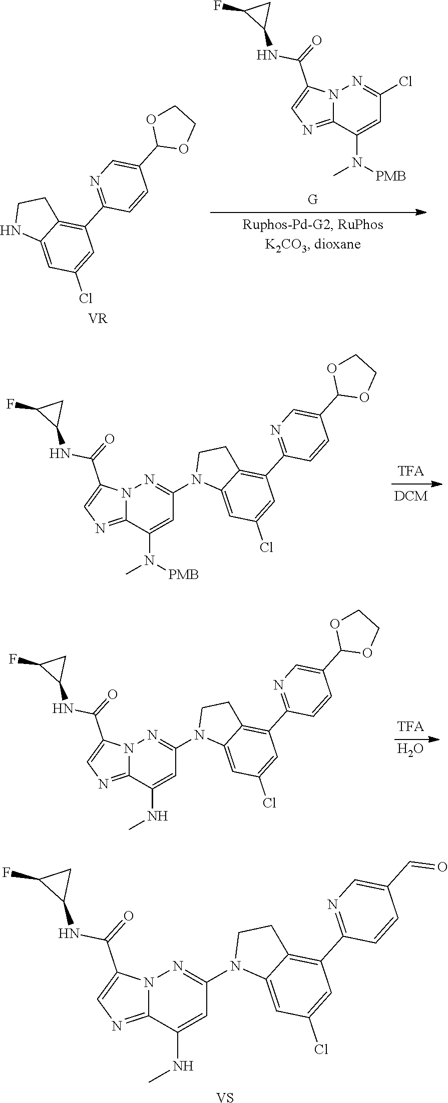

Step 1—6-(4-(5-(1,3-Dioxolan-2-yl)pyridin-2-yl)-6-fluoroindolin-1-yl)-N-((1R,2S)-2-fluorocyclopropyl)-8-((4-methoxybenzyl)(methyl)amino)imidazo[1,2-b]pyridazine-3-carboxamide. To a stirred solution of 4-(5-(1,3-dioxolan-2-yl)pyridin-2-yl)-6-chloroindoline (510 mg, 1.682 mmol, Intermediate VR) and 6-chloro-N-[(1R,2S)-2-fluorocyclopropyl]-8-{[(4-methoxyphenyl)methyl](methyl)amino}imidazo[1,2-b]pyridazine-3-carboxamide (680 mg, 1.680 mmol, Intermediate G) in dioxane (10 mL) were added RuPhos-PdCl-2nd G (131 mg, 0.165 mmol), RuPhos (78 mg, 0.169 mmol) and K₂CO₃ (698 mg, 5.054 mmol) in turns at rt under nitrogen atmosphere. The resulting mixture was stirred for 1 h at 100° C. under nitrogen atmosphere. On completion, the mixture was cooled to rt and concentrated under vacuum. The residue was purified by silica gel column chromatography, eluted with PE/EA (1:1), to afford the title compound (310 mg, 27% yield) as a yellow solid. LC/MS (ESI, m/z): [(M+1)]⁺=670.3.

Step 2—6-(4-(5-(1,3-Dioxolan-2-yl)pyridin-2-yl)-6-fluoroindolin-1-yl)-N-((1R,2S)-2-fluorocyclopropyl)-8-(methylamino)imidazo[1,2-b]pyridazine-3-carboxamide. To a stirred solution of 6-(4-(5-(1,3-dioxolan-2-yl)pyridin-2-yl)-6-fluoroindolin-1-yl)-N-((1R,2S)-2-fluorocyclopropyl)-8-((4-methoxybenzyl)(methyl)amino)imidazo[1,2-b]pyridazine-3-carboxamide (310 mg, 0.464 mmol) in DCM (6 mL) was added TFA (2 mL) dropwise at rt under nitrogen atmosphere. The resulting mixture was stirred for 1 h at rt under nitrogen atmosphere. On completion, the mixture was concentrated under reduced pressure. The residue was purified by trituration with Et₂O (80 mL) to afford the title compound (250 mg, 98% yield) as a yellow oil. LC/MS (ESI, m/z): [(M+1)]⁺=550.3.

Step 3—6-(6-Chloro-4-(5-formylpyridin-2-yl)indolin-1-yl)-N-((1R,2S)-2-fluorocyclopropyl)-8-(methylamino)imidazo[1,2-b]pyridazine-3-carboxamide. To a stirred solution of 6-(4-(5-(1,3-dioxolan-2-yl)pyridin-2-yl)-6-fluoroindolin-1-yl)-N-((1R,2S)-2-fluorocyclopropyl)-8-(methylamino)imidazo[1,2-b]pyridazine-3-carboxamide (250.00 mg, 0.545 mmol) in were added TFA (2 mL) and H₂O (2 mL) dropwise at rt under nitrogen atmosphere. The resulting mixture was stirred for 1 h at 50° C. under nitrogen atmosphere. On completion, the mixture was cooled to rt and basified to pH 8 with saturated Na₂CO₃ (aq.). The precipitated solids were collected by filtration and washed with water (2×100 mL) to give the title compound (200 mg, 72% yield) as a yellow solid. LC/MS (ESI, m/z): [(M+1)]=506.1.

### 6-(4-(((R)-3,3-difluoro-4-(3,9-diazaspiro[5.5]undecan-3-yl)piperidin-1-yl)methyl)indolin-1-yl)-N-((1R,2S)-2-fluorocyclopropyl)-8-(methylamino)imidazo[1,2-b]pyridazine-3-carboxamide (Intermediate VT)

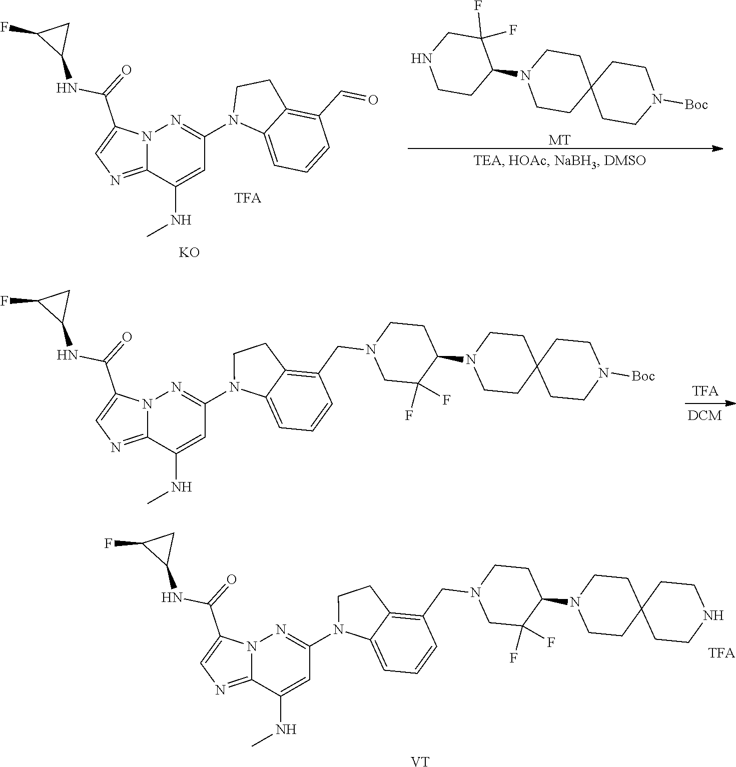

Step 1—Tert-butyl 9-((R)-3,3-difluoro-1-((1-(3-(((1R,2S)-2-fluorocyclopropyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate. To a stirred mixture of tert-butyl 9-[(4S)-3,3-difluoropiperidin-4-yl]-3,9-diazaspiro[5.5]undecane-3-carboxylate (6.20 g, 16.6 mmol, Intermediate MQ) and Et₃N (3.36 g, 33.2 mmol) in DMSO (70 mL) were added N-[(1R,2S)-2-fluorocyclopropyl]-6-(4-formyl-2,3-dihydroindol-1-yl)-8-(methylamino)imidazo[1,2-b]pyridazine-3-carboxamide (7.20 g, 18.3 mmol, Intermediate KO) and HOAc (9.97 g, 166.00 mmol) at rt. The resulting mixture was then stirred for 1 h at 50° C. To the above mixture was added NaBH₃CN (5.22 g, 83.00 mmol) in portions at rt. The resulting mixture was stirred for an additional 1 h at 50° C. On completion, the mixture was cooled to rt and purified directly by reversed-phase flash chromatography (Column: Spherical CIS, 20˜40 μm, 330 g; Mobile Phase A: Water (plus 10 mM NH₄HCO₃), Mobile Phase B: ACN; Flow rate: 80 mL/min; Gradient (B %): 5%˜80%, 4 min; 80%-95%, 20 min; 95%˜95%; 5 min; Detector: 254 nm; the fractions containing desired product were collected at 94% B and concentrated under reduced pressure) to afford the title compound (7 g, 56% yield) as a white solid. LC/MS (ESI, m/z): [(M+1)]-=752.4.

Step 2—6-(4-(((R)-3,3-difluoro-4-(3,9-diazaspiro[5.5]undecan-3-yl)piperidin-1-yl)methyl)indolin-1-yl)-N-((1R,2S)-2-fluorocyclopropyl)-8-(methylamino)imidazo[1,2-b]pyridazine-3-carboxamide trifluoroacetate. To a stirred mixture of tert-butyl 9-[(4R)-3,3-difluoro-1-{[1-(3-{[(1R,2S)-2-fluorocyclopropyl]carbamoyl}-8-(methylamino)imidazo[1,2-b]pyridazin-6-yl)-2,3-dihydroindol-4-yl]methyl}piperidin-4-yl]-3,9-diazaspiro[5.5]undecane-3-carboxylate (7.00 g, 9.31 mmol) in DCM (60 mL) was added TFA (20 mL) at rt. The resulting mixture was stirred for 1 h at rt. On completion, the resulting mixture was concentrated under reduced pressure. The residue was purified by trituration with Et₂O (50 mL) to afford the title compound (6 g, TFA salt) as a brown solid. LC/MS (ESI, m/z): [(M+1)]⁺=652.4.

### 6-(4-(((S)-3,3-difluoro-4-(3,9-diazaspiro[5.5]undecan-3-yl)piperidin-1-yl)methyl)indolin-1-yl)-N-((1R,2S)-2-fluorocyclopropyl)-8-(methylamino)imidazo[1,2-b]pyridazine-3-carboxamide (Intermediate VU)

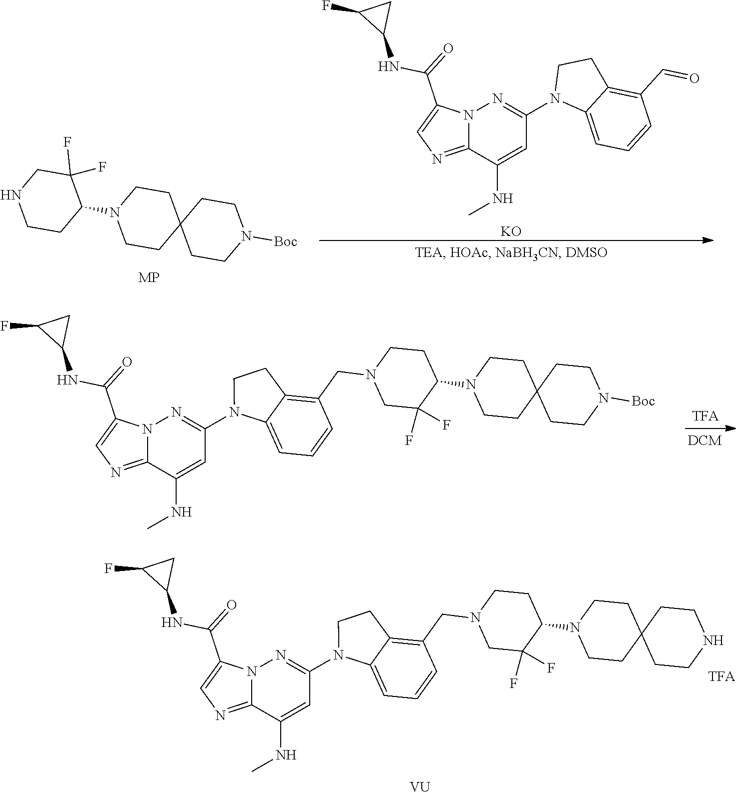

Step 1—Tert-butyl 9-((S)-3,3-difluoro-1-((1-(3-(((1R,2S)-2-fluorocyclopropyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate. To a stirred solution of tert-butyl (R)-9-(3,3-difluoropiperidin-4-yl)-3,9-diazaspiro[5.5]undecane-3-carboxylate (300 mg, 0.803 mmol, Intermediate MP) and TEA (81 mg, 0.803 mmol) in DMSO (5 mL) was added N-((1R,2S)-2-fluorocyclopropyl)-6-(4-formylindolin-1-yl)-8-(methylamino)imidazo[1,2-b]pyridazine-3-carboxamide (317 mg, 0.803 mmol, Intermediate KO) at rt under air atmosphere. To the above mixture was added HOAc (145 mg, 2.409 mmol) at rt. The resulting mixture was stirred for 1 h at 50° C. To the above mixture was added NaBH₃CN (252 mg, 4.015 mmol) at rt. The resulting mixture was stirred for an additional 1 h at 50° C. On completion, the mixture was cooled to rt and purified by reverse phase flash chromatography (WelFlash™ C18-I, 20-40 μm, 330 g; Eluent A: Water (plus 10 mmol/L NH₄HCO₃); Eluent B: ACN; Gradient: 55%-95% B in 25 min; Flow rate: 80 mL/min; Detector: 220/254 nm; desired fractions were collected at 90% B and concentrated under reduced pressure) to afford the title compound (300 mg, 46% yield) as a yellow solid. LC/MS (ESI, m/z): [(M+1)]⁺=752.4.

Step 2—6-(4-(((S)-3,3-difluoro-4-(3,9-diazaspiro[5.5]undecan-3-yl)piperidin-1-yl)methyl)indolin-1-yl)-N-((1R,2S)-2-fluorocyclopropyl)-8-(methylamino)imidazo[1,2-b]pyridazine-3-carboxamide trifluoroacetate. To a stirred solution of tert-butyl 9-((S)-3,3-difluoro-1-((1-(3-(((1R,2S)-2-fluorocyclopropyl)carbamoyl)-8-(methylamino)imidazo[1,2-b]pyridazin-6-yl)indolin-4-yl)methyl)piperidin-4-yl)-3,9-diazaspiro[5.5]undecane-3-carboxylate (300 mg, 0.399 mmol) in DCM (9 mL) was added TEA (3 mL) dropwise at rt. The resulting mixture was concentrated under reduced pressure. The residue was purified by trituration with ethyl ether (10 mL) to afford the title compound (300 mg) as a white solid. LC/MS (ESI, m/z): [(M+1)]⁺=652.3.

### Tert-butyl (S)-4-((3,9-diazaspiro[5.5]undecan-3-yl)methyl)-3,3-difluoropiperidine-1-carboxylate (Intermediate VV)

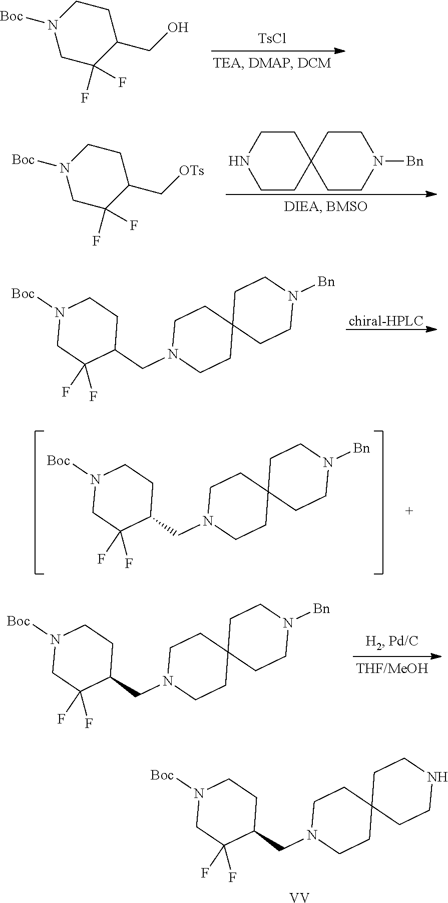

Step 1—tert-butyl 3,3-difluoro-4-{[(4-methylbenzenesulfonyl)oxy]methyl}piperidine-1-carboxylate. To a stirred mixture of tert-butyl 3,3-difluoro-4-(hydroxymethyl)piperidine-1-carboxylate (36.50 g, 145.3 mmol), DMAP (3.55 g, 29.1 mmol) and TEA (29.40 g, 290.5 mmol) in DCM (360 mL) was added TsCl (41.54 g, 217.9 mmol) in portions at 0° C. The resulting mixture was stirred at rt for 1 h under nitrogen atmosphere. On completion, the mixture was concentrated under reduced pressure. The residue was purified by silica gel column chromatography, eluted with petroleum ether/EtOAc (35%), to afford the title compound (57 g, 96% yield) as a colorless oil. ¹H NMR (300 MHz, CDCl₃) δ 7.87-7.76 (m, 2H), 7.38 (d, J=8.0 Hz, 2H), 4.47-4.31 (m, 1H), 4.32-4.06 (m, 2H), 4.03-3.85 (m, 1H), 3.12-2.84 (m, 2H), 2.84-2.64 (m, 1H), 2.48 (s, 3H), 2.39-2.16 (m, 1H), 2.03-1.88 (m, 1H), 1.46 (s, 9H); LC/MS (ESI, m/z): [(M+1-56)]⁺=350.1.

Step 2—Tert-butyl 4-({9-benzyl-3,9-diazaspiro[5.5]undecan-3-yl}methyl)-3,3-difluoropiperidine-1-carboxylate. To a stirred mixture of tert-butyl 3,3-difluoro-4-{[(4-methylbenzenesulfonyl)oxy]methyl}piperidine-1-carboxylate (50.00 g, 123.3 mmol) and 3-benzyl-3,9-diazaspiro[5.5]undecane (33.15 g, 135.7 mmol) in DMSO (250 mL) was added DIEA (47.81 g, 370.0 mmol) at rt under nitrogen atmosphere. The resulting mixture was stirred at 110° C. for 16 h under nitrogen atmosphere. On completion, the mixture was cooled to rt and diluted with water (300 mL). The resulting mixture was extracted with EtOAc (3×200 mL). The combined organic layers were washed with brine (5×300 mL), and dried over anhydrous Na₂SO₄. After filtration, the filtrate was concentrated under reduced pressure. The residue was purified by silica gel column chromatography, eluted with petroleum ether/EtOAc (1:1) to afford the title compound (28 g, 47% yield) as a light yellow solid. ¹H NMR (300 MHz, CDCl₃) δ 7.39-7.30 (m, 4H), 7.32-7.25 (m, 1H), 4.50-3.94 (m, 2H), 3.55 (s, 2H), 3.11-2.86 (m, 1H), 2.86-2.70 (m, 1H), 2.70-2.58 (m, 1H), 2.55-2.36 (m, 6H), 2.36-2.19 (m, 1H), 2.13-1.88 (m, 2H), 1.61-1.37 (m, 20H); LC/MS (ESI, m/z): [(M+1)]-=478.3.

Step 3—Tert-butyl (S)-4-((9-benzyl-3,9-diazaspiro[5.5]undecan-3-yl)methyl)-3,3-difluoropiperidine-1-carboxylate and tert-butyl (R)-4-((9-benzyl-3,9-diazaspiro[5.5]undecan-3-yl)methyl)-3,3-difluoropiperidine-1-carboxylate. Tert-butyl 4-({9-benzyl-3,9-diazaspiro[5.5]undecan-3-yl}methyl)-3,3-difluoropiperidine-1-carboxylate (210 g) was separated by chiral resolution (Column: CHIRALPAK IG, 7*25 cm, 10 μm; Mobile Phase A: CO₂, Mobile Phase B: MeOH: ACN=2: 1; Flow rate: 240 mL/min; Gradient: isocratic 35% B; Column Temperature(° C.): 35; Back Pressure(bar): 100; Wave Length: 254 nm; RT1 (min): 7.13; RT2(min): 9.39; Sample Solvent: MeOH: DCM=1: 1; Injection Volume: 3.5 mL; Number Of Runs: 340). The fractions were collected at 7.13 min (faster eluting peak) were concentrated under vacuum to afford tert-butyl (S)-4-((9-benzyl-3,9-diazaspiro[5.5]undecan-3-yl)methyl)-3,3-difluoropiperidine-1-carboxylate (82 g, 39% yield) as a light yellow solid. The fractions were collected at 9.39 min (slower eluting peak) and concentrated under vacuum tert-butyl (R)-4-((9-benzyl-3,9-diazaspiro[5.5]undecan-3-yl)methyl)-3,3-difluoropiperidine-1-carboxylate (84 g, 40% yield) as a light yellow solid. LC/MS (ESI, m/z): [(M+1)]⁺=478.3 for both enantiomers. The absolute stereochemistry of the enantiomers was assigned arbitrarily.

Step 4—Tert-butyl (S)-4-((3,9-diazaspiro[5.5]undecan-3-yl)methyl)-3,3-difluoropiperidine-1-carboxylate. To a stirred mixture of tert-butyl (S)-4-((9-benzyl-3,9-diazaspiro[5.5]undecan-3-yl)methyl)-3,3-difluoropiperidine-1-carboxylate (17.00 g, 35.59 mmol) in THF (80 mL) and methanol (80 mL) was added Pd/C (6.00 g, 56.4 mmol) in portions at rt under nitrogen atmosphere. The mixture was hydrogenated at rt for 4 h under hydrogen atmosphere using a hydrogen balloon. On completion, the mixture was filtered through a celite pad and concentrated under reduced pressure to afford the title compound (12.7 g, 92% yield) as a grey solid. LC/MS (ESI, m/z): [(M+1)]⁺=388.3.

### (S)-1-(3-chloro-5-(9-((3,3-difluoropiperidin-4-yl)methyl)-3,9-diazaspiro[5.5]undecane-3-carbonyl)-2-methylphenyl)dihydropyrimidine-2,4(1H,3H)-dione (Intermediate VW)

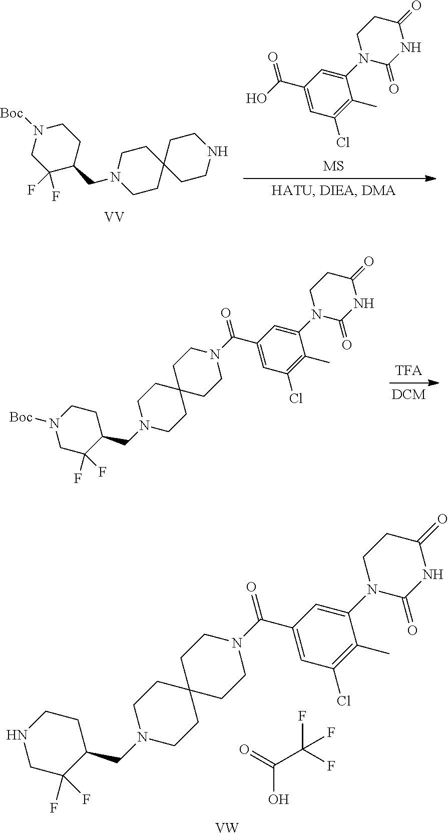

Step 1—Tert-butyl (S)-4-((9-(3-chloro-5-(2,4-dioxotetrahydropyrimidin-1(2H)-yl)-4-methylbenzoyl)-3,9-diazaspiro[5.5]undecan-3-yl)methyl)-3,3-difluoropiperidine-1-carboxylate. To a stirred solution of tert-butyl (S)-4-((3,9-diazaspiro[5.5]undecan-3-yl)methyl)-3,3-difluoropiperidine-1-carboxylate (2.20 g, 5.68 mmol, Intermediate VV) and 3-chloro-5-(2,4-dioxotetrahydropyrimidin-1(2H)-yl)-4-methylbenzoic acid (1.60 g, 5.68 mmol, Intermediate MS) in DMA (20 mL) were added HATU (3.24 g, 8.51 mmol) and DIEA (2.20 g, 17.03 mmol) in turns at rt under nitrogen atmosphere. The resulting mixture was stirred for 1 h at rt under nitrogen atmosphere. Upon completion, the reaction mixture was purified directly by reverse phase Flash chromatography (Column: WelFlash™ C18-I, 20-40 μm, 330 g; Eluent A: Water (10 mmol/L NH₄HCO₃); Eluent B: ACN; Gradient: 40%-60% B in 20 min; Flow rate: 80 mL/min; Detector: 220/254 nm; desired fractions were collected at 50% B and concentrated under reduced pressure) to afford the title compound (2.80 g, 75% yield) as a yellow solid. LC/MS (ESI, m/z): [(M+H)]⁺=652.2.

Step 2—(S)-1-(3-chloro-5-(9-((3,3-difluoropiperidin-4-yl)methyl)-3,9-diazaspiro[5.5]undecane-3-carbonyl)-2-methylphenyl)dihydropyrimidine-2,4(1H,3H)-dione trifluoroacetate. To a stirred solution of tert-butyl (S)-4-((9-(3-chloro-5-(2,4-dioxotetrahydropyrimidin-1(2H)-yl)-4-methylbenzoyl)-3,9-diazaspiro[5.5]undecan-3-yl)methyl)-3,3-difluoropiperidine-1-carboxylate (2.80 g, 4.29 mmol) in DCM (21 mL) was added TFA (7 mL) in portions at rt under nitrogen atmosphere. The resulting mixture was stirred for 1 h at rt under nitrogen atmosphere. Upon completion, the mixture was concentrated under vacuum. The residue was purified by trituration with Et₂O (200 mL) to afford the title compound (3.40 g, 93% yield) as a yellow solid. LC/MS (ESI, m/z): [(M+H)]⁺=552.2.

### Tert-butyl N-(6-chloro-3-{[(1R,2R)-2-methoxycyclobutyl]carbamoyl}imidazo[1,2-b]pyridazin-8-yl)-N-methylcarbamate (Intermediate VX)

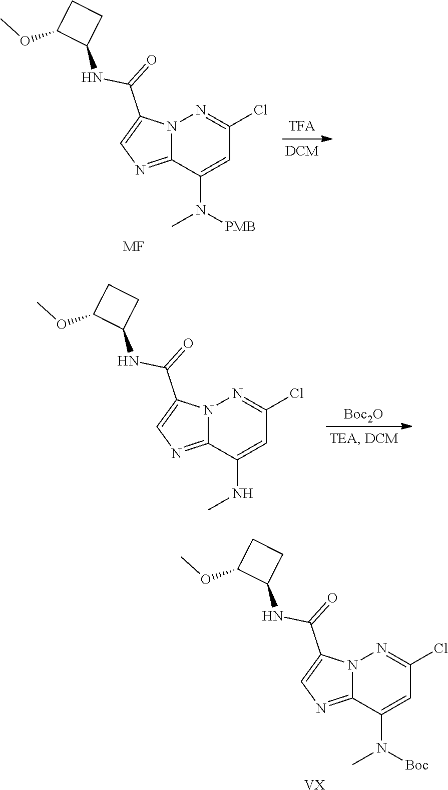

Step 1—6-Chloro-N-[(1R,2R)-2-methoxycyclobutyl]-8-(methylamino)imidazo[1,2-b]pyridazine-3-carboxamide. To a stirred mixture of 6-chloro-N-[(1R,2R)-2-methoxycyclobutyl]-8-{[(4-methoxyphenyl)methyl](methyl)amino}imidazo[1,2-b]pyridazine-3-carboxamide (3 g, 7 mmol, Intermediate MF) in DCM (21 mL) was added TFA (7 mL) at rt. The resulting mixture was stirred for 2 h at rt. On completion, the resulting mixture was concentrated under reduced pressure. The residue was purified by trituration with Et₂O (100 mL) to afford the title compound (2 g, 93% yield) as a brown solid. LC/MS (ESI, m/z): [(M+H)]-=310.1.

Step 2—Tert-butyl N-(6-chloro-3-{[(1R,2R)-2-methoxycyclobutyl]carbamoyl}imidazo[1,2-b]pyridazin-8-yl)-N-methylcarbamate. To a stirred mixture of 6-chloro-N-[(1R,2R)-2-methoxycyclobutyl]-8-(methylamino)imidazo[1,2-b]pyridazine-3-carboxamide (2 g, 7 mmol) and Et₃N (1.96 g, 19.4 mmol) in DCM (20 mL) were added Boc₂O (1.83 g, 8.39 mmol) and DMAP (0.08 g, 0.65 mmol) in portions at if. The resulting mixture was stirred for 30 min at rt. On completion, the mixture was diluted with water (100 mL) and extracted with EtOAc (3×50 mL). The combined organic layers were washed with brine (3×10 mL), and dried over anhydrous Na₂SO₄. After filtration, the filtrate was concentrated under reduced pressure. The residue was purified by silica gel column chromatography, eluted with PE/EA (1:1), to afford the title compound (2.2 g, 83% yield) as a yellow solid. LC/MS (ESI, m/z): [(M+H)]=410.1.

### 6-(4-(4-Formylpiperidin-1-yl)indolin-1-yl)-N-((1R,2R)-2-methoxycyclobutyl)-8-(methylamino)imidazo[1,2-b]pyridazine-3-carboxamide (Intermediate VY)

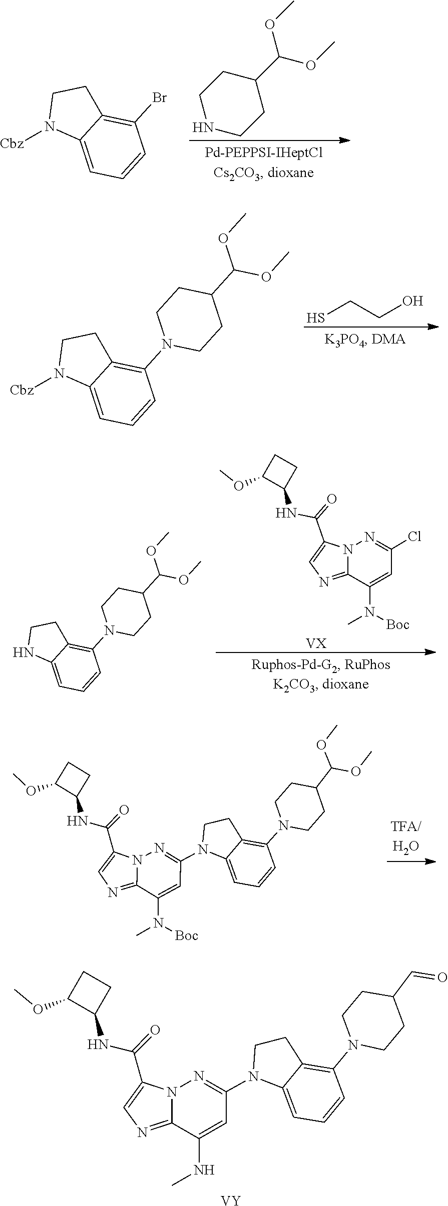

Step 1—Tert-butyl benzyl 4-(4-(dimethoxymethyl)piperidin-1-yl)indoline-1-carboxylate. To a stirred mixture of benzyl 4-bromoindoline-1-carboxylate (1 g, 3 mmol, CAS #2137424-54-3), Cs₂CO₃ (2.94 g, 9.030 mmol) and 4-(dimethoxymethyl)piperidine (0.96 g, 6.020 mmol) in 1,4-dioxane (10 mL) was added 3-chloropyridine{1,3-bis[2,6-bis(heptan-4-yl)phenyl]-4,5-dichloro-2,3-dihydro-1H-imidazol-2-yl}dichloropalladium (0.28 g, 0.241 mmol) at rt under nitrogen atmosphere. The resulting mixture was stirred at 100° C. for 1 h under nitrogen atmosphere. On completion, the mixture was cooled to rt and concentrated under reduced pressure. The residue was purified by reverse phase flash chromatography (Column, Spherical C18, 20-40 μm, 330; Mobile Phase A: Water (plus 10 mM NH₄HCO₃); Mobile Phase B; (ACN); Flow rate: 80 mL/min; Gradient: 30% B-50% B in 20 min; Detector, UV 254 nm; the fractions containing desired product were collected at 50% B and concentrated under reduced pressure) to afford the title compound (1.16 g, 94% yield) as a brown solid. LC/MS (ESI, m/z): [(M+H)]⁺=411.2.

Step 2—4-(4-(Dimethoxymethyl)piperidin-1-yl)indoline. To a stirred mixture of benzyl 4-(4-(dimethoxymethyl)piperidin-1-yl)indoline-1-carboxylate (500 mg, 1.22 mmol) and K₃PO₄ (1034.14 mg, 4.872 mmol) in N,N-dimethylacetamide (7 mL) was added 2-mercaptoethan-1-ol (380 mg, 4.87 mmol) at rt under nitrogen atmosphere. The resulting mixture was stirred at 70° C. for 16 h under nitrogen atmosphere. On completion, the mixture was cooled to rt and concentrated under reduced pressure. The residue was purified by reverse phase flash chromatography (Column, Spherical C18, 20-40 μm, 330; Mobile Phase A: Water (plus 10 mM NH₄HCO₃); Mobile Phase B; (ACN); Flow rate: 80 mL/min; Gradient: 30% B-50% B in 20 min; Detector, UV 254 nm; the fractions containing desired product were collected at 36% B and concentrated under reduced pressure) to afford the title compound (285 mg, 85% yield) as an off-white solid. LC/MS (ESI, m/z): [(M+H)]⁺=277.2.

Step 3—Tert-butyl (6-(4-(4-(dimethoxymethyl) piperidin-1-yl)indolin-1-yl)-3-(((1R,2R)-2-methoxycyclobutyl)carbamoyl)imidazo[1,2-b]pyridazin-8-yl)(methyl)carbamate. To a stirred mixture of 4-(4-(dimethoxymethyl)piperidin-1-yl)indoline (350 mg, 1.27 mmol), tert-butyl (6-chloro-3-(((1R,2R)-2-methoxycyclobutyl)carbamoyl)imidazo[1,2-b]pyridazin-8-yl)(methyl)carbamate (519 mg, 1.27 mmol, Intermediate VX) and K₂CO₃ (525 mg, 3.80 mmol) in 1,4-dioxane (7 mL) were added RuPhos (118 mg, 0.253 mmol) and RuPhos Pd G₂ (197 mg, 0.253 mmol) at rt under nitrogen atmosphere. The resulting mixture was stirred at 100° C. for 2 h under nitrogen atmosphere. Upon completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L FA); Eluent B: ACN; Gradient: 35%-55% B in 15 min; Flow rate: 80 mL/min; Detector: 220/254 nm; desired fractions were collected at 50% B and concentrated under reduced pressure) to afford the title compound (370 mg, 45% yield) as a brown solid. LC/MS (ESI, m/z): [(M+H)]⁺=650.3.

Step 4—6-(4-(4-Formylpiperidin-1-yl)indolin-1-yl)-N-((1R,2R)-2-methoxycyclobutyl)-8-(methylamino)imidazo[1,2-b]pyridazine-3-carboxamide. A solution of tert-butyl (6-(4-(4-(dimethoxymethyl)piperidin-1-yl)indolin-1-yl)-3-(((1R,2R)-2-methoxycyclobutyl)carbamoyl)imidazo[1,2-b]pyridazin-8-yl)(methyl)carbamate (370 mg, 0.569 mmol) in H₂O (2 mL) and trifluoroacetic acid (2 mL) was stirred at 50° C. for 1 h under nitrogen atmosphere. On completion, the mixture was cooled to rt and basified to pH 8 with Na₂CO₃ (aq.). The resulting mixture was extracted with EtOAc (3×50 mL), and the combined organic layers were dried over anhydrous Na₂SO₄. After filtration, the filtrate was concentrated under reduced pressure to afford the title compound (200 mg) as a yellow solid. LC/MS (ESI, m/z): [(M+H)]⁺=504.3.

### 1-(3-Chloro-6-(4-((3,3-difluoropiperidin-4-yl)methyl)piperazine-1-carbonyl)-2-methylpyridin-4-yl)dihydropyrimidine-2,4(1H,3H)-dione (Intermediate VZ)

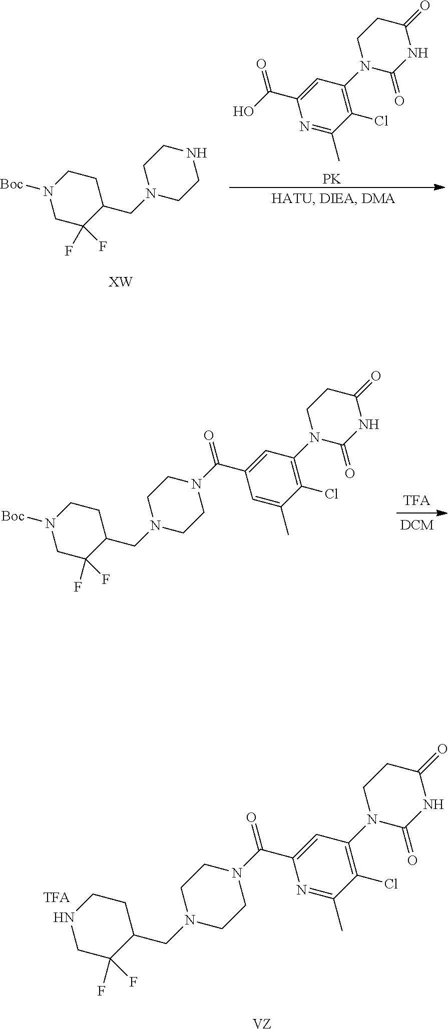

Step 1—Tert-butyl 4-({4-[5-chloro-4-(2,4-dioxo-1,3-diazinan-1-yl)-6-methylpyridine-2-carbonyl]piperazin-1-yl}methyl)-3,3-difluoropiperidine-1-carboxylate. To a stirred mixture of tert-butyl 3,3-difluoro-4-(piperazin-1-ylmethyl)piperidine-1-carboxylate (300 mg, 0.939 mmol, Intermediate XW) and 5-chloro-4-(2,4-dioxotetrahydropyrimidin-1(2H)-yl)-6-methylpicolinic acid (266.44 mg, 0.939 mmol, Intermediate PK) in DMA (5 mL) was added DIEA (364.19 mg, 2.817 mmol) and HATU (535.71 mg, 1.408 mmol) at rt under nitrogen atmosphere. The resulting mixture was stirred at rt for 1 h under nitrogen atmosphere. On completion, the resulting mixture was concentrated under reduced pressure. The residue was purified by reverse flash chromatography (column, C18 silica gel; mobile phase A: water (10 mmol/L NH₄HCO₃), mobile phase B: ACN, 30% to 50% gradient in 30 min; detector, UV 254 nm; the fractions were collected at 45% and concentrated under reduced pressure to afford) the title compound (400 mg, 73% yield) as a white solid. LC/MS (ESI, m/z): [(M+H)]⁺=585.2.

Step 2—1-(3-chloro-6-{4-[(3,3-difluoropiperidin-4-yl)methyl]piperazine-1-carbonyl}-2-methylpyridin-4-yl)-1,3-diazinane-2,4-dione trifluoroacetate. To a stirred solution of tert-butyl 4-({4-[5-chloro-4-(2,4-dioxo-1,3-diazinan-1-yl)-6-methylpyridine-2-carbonyl]piperazin-1-yl}methyl)-3,3-difluoropiperidine-1-carboxylate (400 mg, 0.684 mmol) in DCM (6 mL) was added TFA (3 mL) dropwise at rt under nitrogen atmosphere. The resulting mixture was stirred at rt for 1 h under nitrogen atmosphere. On completion, the resulting mixture was concentrated under reduced pressure. The residue was purified by trituration with ethyl ether (15 mL) to afford the title compound (380 mg, 95% yield) as a white solid. LC/MS (ESI, m/z): [(M+H)]⁺=485.2.

### 6-(4-(4-((5,5-Difluoro-2,7-diazaspiro[3.5]nonan-2-yl)methyl)piperidin-1-yl)indolin-1-yl)-N-((1R,2R)-2-methoxycyclobutyl)-8-(methylamino)imidazo[1,2-b]pyridazine-3-carboxamide (Intermediate WA)

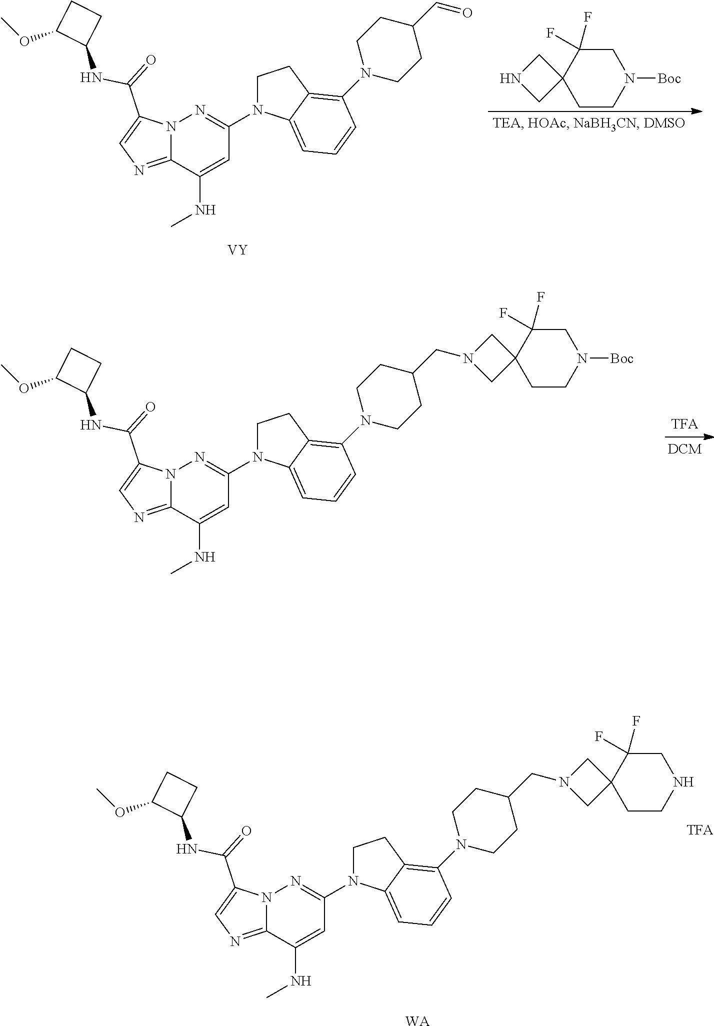

Step 1—Tert-butyl 5,5-difluoro-2-((1-(1-(3-(((1R,2R)-2-methoxycyclobutyl)carbamoyl)-8-(methylamino)imidazo[1,2-b]pyridazin-6-yl)indolin-4-yl)piperidin-4-yl)methyl)-2,7-diazaspiro[3.5]nonane-7-carboxylate. To a stirred solution of 6-(4-(4-formylpiperidin-1-yl)indolin-1-yl)-N-((1R,2R)-2-methoxycyclobutyl)-8-(methylamino)imidazo[1,2-b]pyridazine-3-carboxamide (300 mg, 0.596 mmol, Intermediate VY) in DMSO (6 mL) were added TEA (60 mg, 0.596 mmol), tert-butyl 5,5-difluoro-2,7-diazaspiro[3.5]nonane-7-carboxylate (156 mg, 0.596 mmol) and HOAc (107 mg, 1.788 mmol) at rt. The resulting mixture was stirred at 50° C. for 2 h. The mixture was cooled down to rt. Then NaBH₃CN (150 mg, 2.38 mmol) was added in portions at rt. The resulting mixture was stirred at 50° C. for 2 h. Upon completion, the mixture was purified by reverse phase Flash chromatography (Column: WelFlash™ C18-I, 20-40 μm, 330 g; Eluent A: Water (plus 10 mmol/L NH₄HCO₃); Eluent B: ACN; Gradient: 50%-95% B in 30 min; Flow rate: 80 mL/min; Detector: 220/254 nm; desired fractions were collected at 95% B and concentrated under reduced pressure) to afford the title compound (350 mg, 78% yield) as a light yellow solid. LC/MS (ESI, m/z): [(M+1)]⁺=750.3.

Step 2—6-(4-(4-((5,5-Difluoro-2,7-diazaspiro[3.5]nonan-2-yl)methyl)piperidin-1-yl)indolin-1-yl)-N-((1R,2R)-2-methoxycyclobutyl)-8-(methylamino)imidazo[1,2-b]pyridazine-3-carboxamide trifluoroacetate. To a stirred solution of tert-butyl 5,5-difluoro-2-((1-(1-(3-(((1R,2R)-2-methoxycyclobutyl)carbamoyl)-8-(methylamino)imidazo[1,2-b]pyridazin-6-yl)indolin-4-yl)piperidin-4-yl)methyl)-2,7-diazaspiro[3.5]nonane-7-carboxylate (100 mg, 0.133 mmol) in DCM (3 mL) was added TFA (1 mL) at rt. The resulting mixture was stirred at rt for 1 h. Upon completion, the mixture was concentrated under vacuum. The residue was purified by trituration with ethyl ether (5 mL) to afford the title compound (80 mg, TFA salt) as a light yellow solid. LC/MS (ESI, m/z): [(M+1)]⁺=650.3.

### Tert-butyl (6-(4-(3-formylbicyclo[1.1.1]pentan-1-yl)indolin-1-yl)-3-(((1R,2R)-2-methoxycyclobutyl)carbamoyl)imidazo[1,2-b]pyridazin-8-yl)(methyl)carbamate (Intermediate WB)

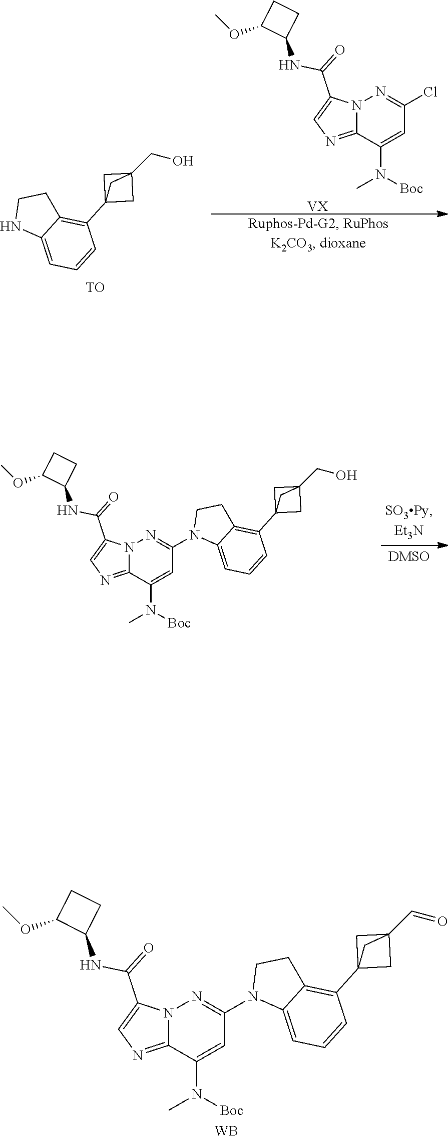

Step 1—Tert-butyl N-(6-{4-[3-(hydroxymethyl)bicyclo[1.1.1]pentan-1-yl]-2,3-dihydroindol-1-yl}-3-{[(1R,2R)-2-methoxycyclobutyl]carbamoyl}imidazo[1,2-b]pyridazin-8-yl)-N-methylcarbamate. To a stirred mixture of [3-(2,3-dihydro-1H-indol-4-yl)bicyclo[1.1.1]pentan-1-yl]methanol (700 mg, 3.251 mmol, Intermediate TO), tert-butyl N-(6-chloro-3-{[(1R,2R)-2-methoxycyclobutyl]carbamoyl}imidazo[1,2-b]pyridazin-8-yl)-N-methylcarbamate (1.33 g, 3.25 mmol, Intermediate VX) and K₂CO₃ (1.35 g, 9.75 mmol) in dioxane (15 mL) were added RuPhos (303 mg, 0.650 mmol) and RuPhos-PdCl-2nd G (506 mg, 0.650 mmol) at rt under nitrogen atmosphere. The resulting mixture was stirred for 1 h at 110° C. under nitrogen atmosphere. On completion, the mixture was cooled to rt and purified by reversed-phase flash chromatography (Column: WelFlash™ C18-I, 20-40 μm, 330 g; Eluent A: Water (plus 10 mmol/L NH₄HCO₃); Eluent B: ACN; Gradient: 45%-65% B in 15 min; Flow rate: 80 mL/min; Detector: 220/254 nm; desired fractions were collected at 57% B and concentrated under reduced pressure) to afford the title compound (850 mg, 44% yield) as a green solid. LC/MS (ESI, m/z): [(M+H)]⁺=589.3.

Step 2—Tert-butyl (6-(4-(3-formylbicyclo[1.1.1]pentan-1-yl)indolin-1-yl)-3-(((1R,2R)-2-methoxycyclobutyl)carbamoyl)imidazo[1,2-b]pyridazin-8-yl)(methyl)carbamate. To a stirred mixture of tert-butyl N-(6-{4-[3-(hydroxymethyl)bicyclo[1.1.1]pentan-1-yl]-2,3-dihydroindol-1-yl}-3-{[(1R,2R)-2-methoxycyclobutyl]carbamoyl}imidazo[1,2-b]pyridazin-8-yl)-N-methylcarbamate (80 mg, 0.136 mmol) and TEA (112 mg, 1.107 mmol) in DMSO (3 mL) was added SO₃-pyridine (88 mg, 0.553 mmol) at 0° C. under nitrogen atmosphere. The resulting mixture was stirred at rt for 1 h under nitrogen atmosphere. On completion, the title compound mixture was used in the next step directly without further purification. LC/MS (ESI, m/z): [(M+H)]⁺=587.3.

### 6-(4-(3-((5,5-Difluoro-2,7-diazaspiro[3.5]nonan-2-yl)methyl)bicyclo[1.1.1]pentan-1-yl)indolin-1-yl)-N-((1R,2R)-2-methoxycyclobutyl)-8-(methylamino)imidazo[1,2-b]pyridazine-3-carboxamide (Intermediate WC)

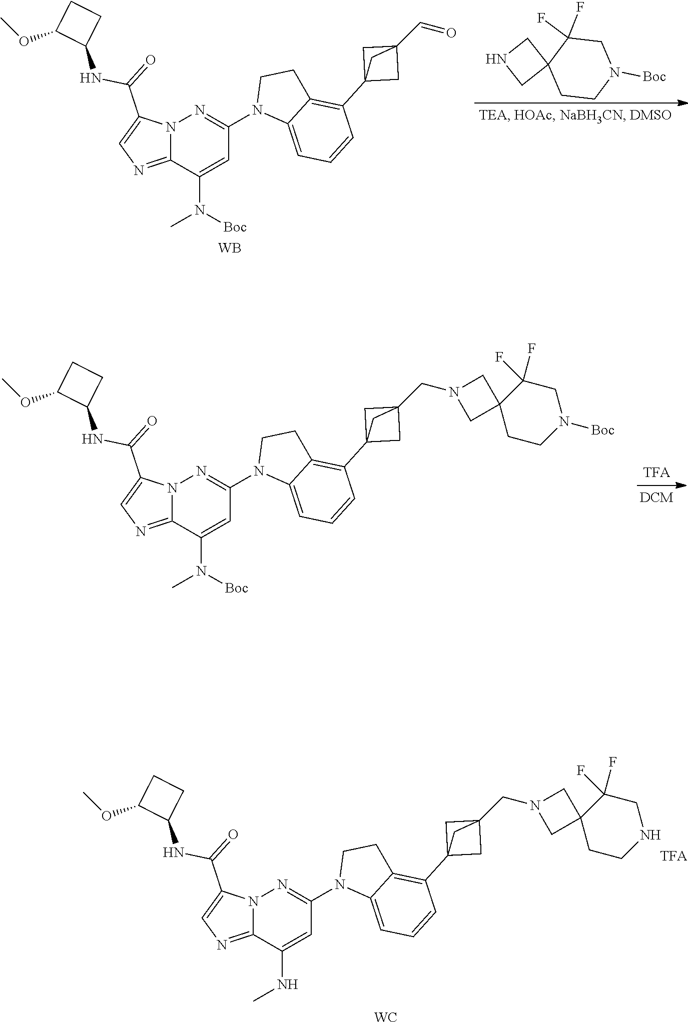

Step 1—Tert-butyl 2-{[3-(1-{8-[(tert-butoxycarbonyl)(methyl)amino]-3-{[(1R,2R)-2-methoxycyclobutyl]carbamoyl}imidazo[1,2-b]pyridazin-6-yl}-2,3-dihydroindol-4-yl)bicyclo[1.1.1]pentan-1-yl]methyl}-5,5-difluoro-2,7-diazaspiro[3.5]nonane-7-carboxylate. To a stirred mixture of tert-butyl N-[6-(4-{3-formylbicyclo[1.1.1]pentan-1-yl}-2,3-dihydroindol-1-yl)-3-{[(1R,2R)-2-methoxycyclobutyl]carbamoyl}imidazo[1,2-b]pyridazin-8-yl]-N-methylcarbamate (80 mg, 0.14 mmol, Intermediate WB) and TEA (0.06 mL, 0.408 mmol) in DMSO (1 mL) were added tert-butyl 5,5-difluoro-2,7-diazaspiro[3.5]nonane-7-carboxylate (36 mg, 0.137 mmol) and HOAc (0.04 mL, 0.680 mmol) at rt. The resulting mixture was stirred at 50° C. for 30 min. To the above mixture was added NaBH₃CN (26 mg, 0.414 mmol). The resulting mixture was stirred at 50° C. for an additional 1 h. On completion, the mixture cooled to rt and was purified directly by reverse phase flash chromatography (Column: WelFlash™ C18-I, 20-40 μm, 120 g; Eluent A: Water (plus 10 mmol/L NH₄HCO₃); Eluent B: ACN; Gradient: 65%-95% B in 20 min; Flow rate: 60 mL/min; Detector: 220/254 nm; desired fractions were collected at 93% B and concentrated under reduced pressure) to afford the title compound (100 mg, 88% yield) as a light yellow solid. LC/MS (ESI, m/z): [(M+H)]=833.3.

Step 2—6-(4-(3-((5,5-Difluoro-2,7-diazaspiro[3.5]nonan-2-yl)methyl)bicyclo[1.1.1]pentan-1-yl)indolin-1-yl)-N-((1R,2R)-2-methoxycyclobutyl)-8-(methylamino)imidazo[1,2-b]pyridazine-3-carboxamide trifluoroacetate. To a stirred mixture of tert-butyl 2-{[3-(1-{8-[(tert-butoxycarbonyl)(methyl)amino]-3-{[(1R,2R)-2-methoxycyclobutyl]carbamoyl}imidazo[1,2-b]pyridazin-6-yl}-2,3-dihydroindol-4-yl)bicyclo[1.1.1]pentan-1-yl]methyl}-5,5-difluoro-2,7-diazaspiro[3.5]nonane-7-carboxylate (100 mg, 0.120 mmol) in DCM (6 mL) was added TFA (2 mL) at rt. The resulting mixture was stirred at rt for 1 h. On completion, the mixture was concentrated under reduced pressure. The residue was purified by trituration with Et₂O (10 mL) to afford the title compound (87 mg, TFA salt) as a light yellow solid. LC/MS (ESI, m/z): [(M+H)]=633.3.

### 6-(4-(3-((4-(3,3-Difluoropiperidin-4-yl)piperazin-1-yl)methyl)bicyclo[1.1.1]pentan-1-yl)indolin-1-yl)-N-((1R,2R)-2-methoxycyclobutyl)-8-(methylamino)imidazo[1,2-b]pyridazine-3-carboxamide (Intermediate WD)

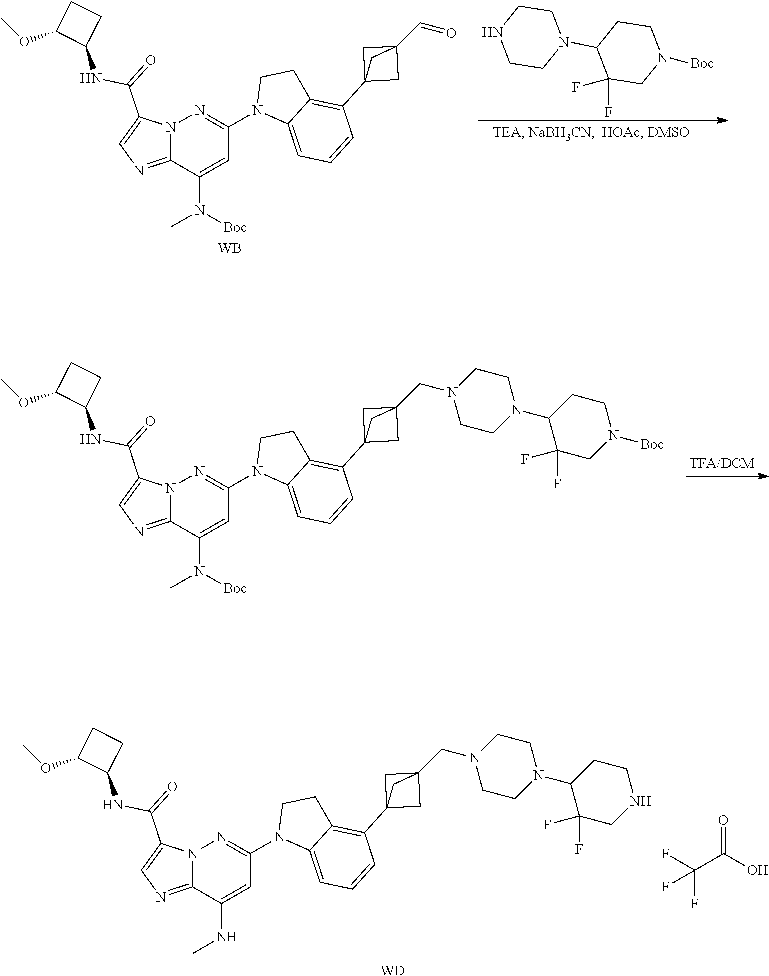

Step 1—Tert-butyl 4-(4-((3-(1-(8-((tert-butoxycarbonyl)(methyl)amino)-3-(((1R,2R)-2-methoxycyclobutyl)carbamoyl)imidazo[1,2-b]pyridazin-6-yl)indolin-4-yl)bicyclo[1.1.1]pentan-1-yl)methyl)piperazin-1-yl)-3,3-difluoropiperidine-1-carboxylate

To a stirred solution of tert-butyl N-[6-(4-{3-formylbicyclo[1.1.1]pentan-1-yl}-2,3-dihydroindol-1-yl)-3-{[(1R,2R)-2-methoxycyclobutyl]carbamoyl}imidazo[1,2-b]pyridazin-8-yl]-N-methylcarbamate (100 mg, 0.170 mmol, Intermediate WB) and tert-butyl 3,3-difluoro-4-(piperazin-1-yl)piperidine-1-carboxylate (62.46 mg, 0.204 mmol, CAS #2384221-09-2) in DMSO was added HOAc (30.71 mg, 0.510 mmol) dropwise at rt. The resulting mixture was stirred at 50° C. for 30 min under nitrogen atmosphere. The mixture was then allowed to cool down to rt. To the above mixture was added NaBH₃CN (32.13 mg, 0.510 mmol) in portions over 5 min at rt under nitrogen atmosphere. The resulting mixture was stirred at 50° C. for an additional 2 h. Upon completion, the reaction mixture was purified directly by reverse phase flash chromatography (Column: WelFlash™ C18-I, 20-40 μm, 120 g; Eluent A: Water (plus 10 mmol/L NH₄HCO₃); Eluent B: ACN; Gradient: 70%-95% B in 25 min; Flow rate: 60 mL/min; Detector: 220/254 nm; desired fractions were collected at 90% B and concentrated under reduced pressure) to afford the title compound (100 mg, 66% yield) as a yellow solid. LC/MS (ESI, m/z): [(M+1)]=876.4.

Step 2—6-(4-(3-((4-(3,3-Difluoropiperidin-4-yl)piperazin-1-yl)methyl)bicyclo[1.1.1]pentan-1-yl)indolin-1-yl)-N-((1R,2R)-2-methoxycyclobutyl)-8-(methylamino)imidazo[1,2-b]pyridazine-3-carboxamide trifluoroacetate

To a stirred solution of tert-butyl 4-(4-((3-(1-(8-((tert-butoxycarbonyl)(methyl)amino)-3-(((1R,2R)-2-methoxycyclobutyl)carbamoyl)imidazo[1,2-b]pyridazin-6-yl)indolin-4-yl)bicyclo[1.1.1]pentan-1-yl)methyl)piperazin-1-yl)-3,3-difluoropiperidine-1-carboxylate (100 mg, 0.114 mmol) in DCM (2 mL) was added TFA (1 mL) dropwise at rt under nitrogen atmosphere. The resulting mixture was stirred at rt for 1 h under nitrogen atmosphere. Upon completion, the mixture was concentrated under reduced pressure. The mixture was purified by trituration with Et₂O (20 mL) to afford the title compound (88 mg, 99% yield) as an off-white solid. LC/MS (ESI, m/z): [(M+1)]⁺=676.4.

### 6-(4-((1,1-Difluoro-3,9-diazaspiro[5.5]undecan-3-yl)methyl)indolin-1-yl)-N-((1R,2R)-2-methoxycyclobutyl)-8-(methylamino)imidazo[1,2-b]pyridazine-3-carboxamide (Intermediate WE)

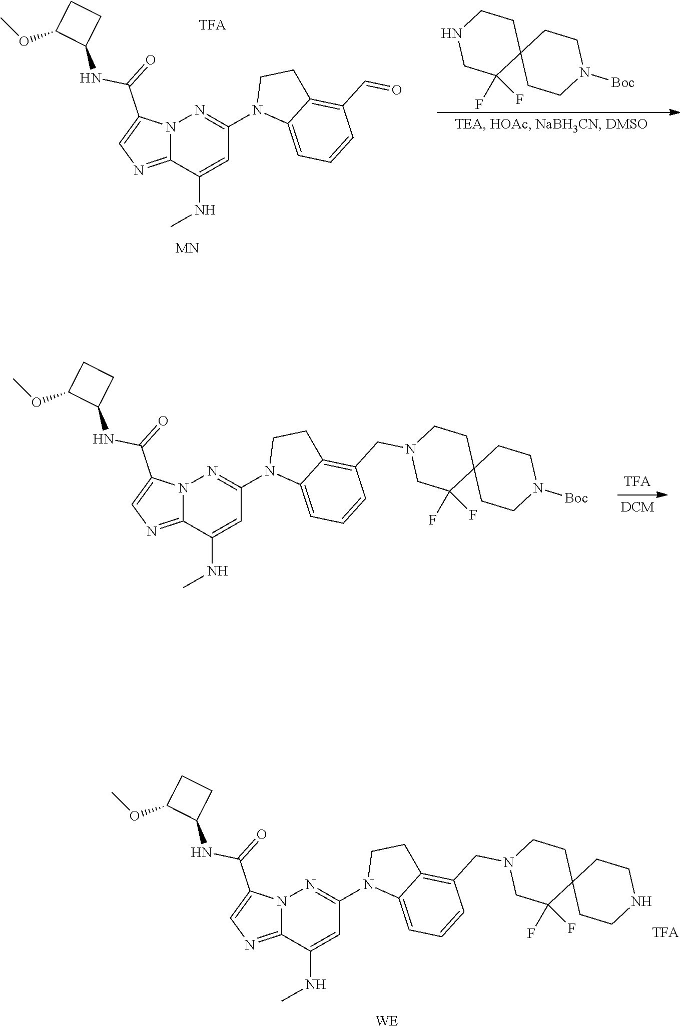

Step 1—Tert-butyl 7,7-difluoro-9-((1-(3-(((1R,2R)-2-methoxycyclobutyl)carbamoyl)-8-(methylamino)imidazo[1,2-b]pyridazin-6-yl)indolin-4-yl)methyl)-3,9-diazaspiro[5.5]undecane-3-carboxylate. To a stirred mixture of tert-butyl 7,7-difluoro-3,9-diazaspiro[5.5]undecane-3-carboxylate (1.04 g, 3.57 mmol) and Et₃N (1.49 mL, 10.701 mmol) in DMSO (15 mL) were added 6-(4-formylindolin-1-yl)-N-((1R,2R)-2-methoxycyclobutyl)-8-(methylamino)imidazo[1,2-b]pyridazine-3-carboxamide trifluoroacetate (1.84 g, 3.57 mmol, Intermediate MN) and AcOH (1.07 mL, 17.8 mmol) at rt under nitrogen atmosphere. The resulting mixture was stirred at 50° C. for 0.5 h under nitrogen atmosphere. To the above mixture was added sodium cyanoboranuide (0.67 g, 10.701 mmol) at rt. The resulting mixture was stirred at 50° C. for an additional 2 h. On completion, the mixture was cooled to rt and concentrated under reduced pressure. The residue was purified by trituration with H₂O (20 mL) to afford the title compound (1.8 g, 72% yield) as a white solid. LC/MS (ESI, m/z): [(M+H)]⁺=695.4.

Step 2—6-(4-((1,1-Difluoro-3,9-diazaspiro[5.5]undecan-3-yl)methyl)indolin-1-yl)-N-((1R,2R)-2-methoxycyclobutyl)-8-(methylamino)imidazo[1,2-b]pyridazine-3-carboxamide trifluoroacetate. To a stirred solution of tert-butyl 7,7-difluoro-9-((1-(3-(((1R,2R)-2-methoxycyclobutyl)carbamoyl)-8-(methylamino)imidazo[1,2-b]pyridazin-6-yl)indolin-4-yl)methyl)-3,9-diazaspiro[5.5]undecane-3-carboxylate (1.8 g, 2.6 mmol) in DCM (10 mL) was added trifluoroacetic acid (10 mL) at rt under nitrogen atmosphere. The resulting mixture was stirred for 1 h at rt under nitrogen atmosphere. On completion, the mixture was concentrated under reduced pressure. The residue was purified by trituration with Et₂O (20 mL) to afford the title compound (2 g) as a white solid. LC/MS (ESI, m/z): [(M+H)]⁺=595.3.

### 6-(4-((1,1-Difluoro-9-(piperidin-4-ylmethyl)-3,9-diazaspiro[5.5]undecan-3-yl)methyl)indolin-1-yl)-N-((1R,2R)-2-methoxycyclobutyl)-8-(methylamino)imidazo[1,2-b]pyridazine-3-carboxamide (Intermediate WF)

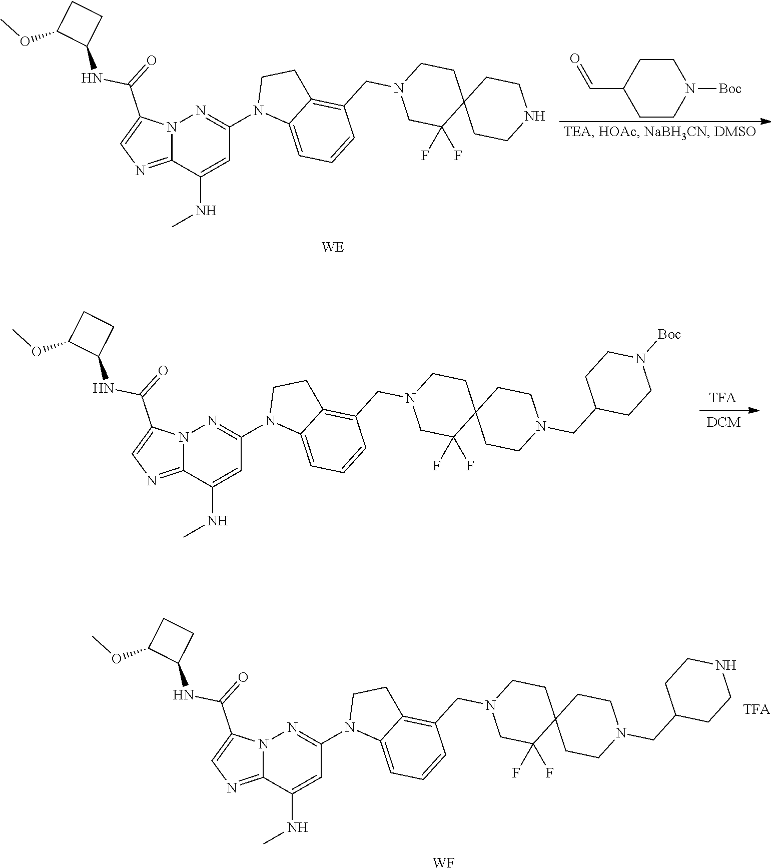 [SMILES: CNc1cc(N2CCc3c(CN4CCC5(CCNCC5)C(F)(F)C4)cccc32)nn2c(C(=O)N[C@@H]3CC[C@H]3OC)cnc12 ; CC(C)(C)OC(=O)N1CCC(C=O)CC1 ; "WE" ; CNc1cc(N2CCc3c(CN4CCC5(CCN(CC6CCN(C(=O)OC(C)(C)C)CC6)CC5)C(F)(F)C4)cccc32)nn2c(C(=O)N[C@@H]3CC[C@H]3OC)cnc12 ; CNc1cc(N2CCc3c(CN4CCC5(CCN(CC6CCNCC6)CC5)C(F)(F)C4)cccc32)nn2c(C(=O)N[C@@H]3CC[C@H]3OC)cnc12 ; "WF"]

Step 1—Tert-butyl 4-((7,7-difluoro-9-((1-(3-(((1R,2R)-2-methoxycyclobutyl)carbamoyl)-8-(methylamino)imidazo[1,2-b]pyridazin-6-yl)indolin-4-yl)methyl)-3,9-diazaspiro[5.5]undecan-3-yl)methyl)piperidine-1-carboxylate. To a stirred mixture of 6-(4-((1,1-difluoro-3,9-diazaspiro[5.5]undecan-3-yl)methyl)indolin-1-yl)-N-((1R,2R)-2-methoxycyclobutyl)-8-(methylamino)imidazo[1,2-b]pyridazine-3-carboxamide trifluoroacetate (500 mg, 0.722 mmol, Intermediate WE) and Et₃N (0.3 mL, 2.2 mmol) in DMSO (8 mL) were added AcOH (0.4 mL, 3.6 mmol) and tert-butyl 4-formylpiperidine-1-carboxylate (154 mg, 0.722 mmol) at rt under nitrogen atmosphere. The resulting mixture was stirred at 50° C. for 0.5 h under nitrogen atmosphere. To the above mixture was added sodium cyanoboranuide (136 mg, 2.166 mmol) at rt. The resulting mixture was stirred at 50° C. for an additional 2 h. Upon completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH₄CO₃); Eluent B: ACN; Gradient: 35%-55% B in 15 min; Flow rate: 80 mL/min; Detector: 220/254 nm; desired fractions were collected at 51% B and concentrated under reduced pressure) to afford the title compound (320 mg, 56% yield) as a white solid. LC/MS (ESI, m/z): [(M+H)]⁺=792.4.

Step 2—6-(4-((1,1-Difluoro-9-(piperidin-4-ylmethyl)-3,9-diazaspiro[5.5]undecan-3-yl)methyl)indolin-1-yl)-N-((1R,2R)-2-methoxycyclobutyl)-8-(methylamino)imidazo[1,2-b]pyridazine-3-carboxamide trifluoroacetate. To a stirred solution of tert-butyl 4-((7,7-difluoro-9-((1-(3-(((1R,2R)-2-methoxycyclobutyl)carbamoyl)-8-(methylamino)imidazo[1,2-b]pyridazin-6-yl)indolin-4-yl)methyl)-3,9-diazaspiro[5.5]undecan-3-yl)methyl)piperidine-1-carboxylate (320 mg, 0.404 mmol) in DCM (4 mL) was added trifluoroacetic acid (2 mL) at rt under nitrogen atmosphere. The resulting mixture was stirred at rt for additional 1 h. Upon completion, the mixture was concentrated under reduced pressure. The residue was purified by trituration with Et₂O (10 mL) to afford the title compound (540 mg) as a white solid. LC/MS (ESI, m/z): [(M+H)]$=692.4.

### Tert-butyl 2-(piperidin-4-yloxy)-7-azaspiro[3.5]nonane-7-carboxylate (Intermediate WG)

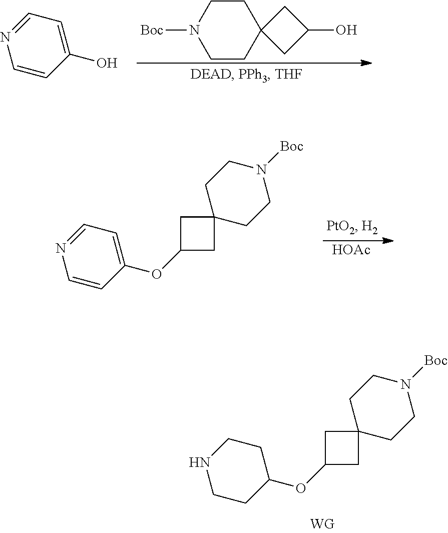 [SMILES: Oc1ccncc1 ; CC(C)(C)OC(=O)N1CCC2(CC1)CC(O)C2 ; CC(C)(C)OC(=O)N1CCC2(CC1)CC(Oc1ccncc1)C2 ; CC(C)(C)OC(=O)N1CCC2(CC1)CC(OC1CCNCC1)C2 ; "HOAc"]

Step 1:—Tert-butyl 2-(pyridin-4-yloxy)-7-azaspiro[3.5]nonane-7-carboxylate. To a stirred mixture of 4-hydroxypyridine (1 g, 10 mmol), tert-butyl 2-hydroxy-7-azaspiro[3.5]nonane-7-carboxylate (2.54 g, 10.5 mmol) and PPh₃ (4.14 g, 15.8 mmol) in THF (30 mL) was added N-[(ethoxycarbonyl)imino]ethoxyformamide (2.75 g, 15.8 mmol) at 0° C. under nitrogen atmosphere. The resulting mixture was stirred at rt for an additional 1 h. On completion, the mixture was concentrated under reduced pressure. The residue was purified by silica gel column chromatography, eluted with PE/EA (1:1), to afford the title compound (2.1 g, 63% yield) as a white solid. LC/MS (ESI, m/z): [(M+H)]=319.2.

Step 2—Tert-butyl 2-(piperidin-4-yloxy)-7-azaspiro[3.5]nonane-7-carboxylate. To a stirred mixture of tert-butyl 2-(pyridin-4-yloxy)-7-azaspiro[3.5]nonane-7-carboxylate (2.1 g, 6.3 mmol) in AcOH (20 mL) was added Platinum(IV)oxide (4.28 g) at rt under nitrogen atmosphere. The resulting mixture was purged with hydrogen three times and stirred for 16 h at rt under hydrogen atmosphere. On completion, the mixture was filtered, the filter cake was washed with ACN (3×20 mL). The filtrate was concentrated under reduced pressure. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 20%-45% B in 25 min; Flow rate: 80 mL/min; Detector: 220/254 nm; desired fractions were collected at 30% B and concentrated under reduced pressure) to afford the title compound (640 mg, 28% yield) as a white solid. LC/MS (ESI, m/z): [(M+H)]f=325.2.

### 6-{4-[(4-{7-Azaspiro[3.5]nonan-2-yloxy}piperidin-1-yl)methyl]-2,3-dihydroindol-1-yl}-N-[(1R,2R)-2-methoxycyclobutyl]-8-(methylamino)imidazo[1,2-b]pyridazine-3-carboxamide (Intermediate WH)

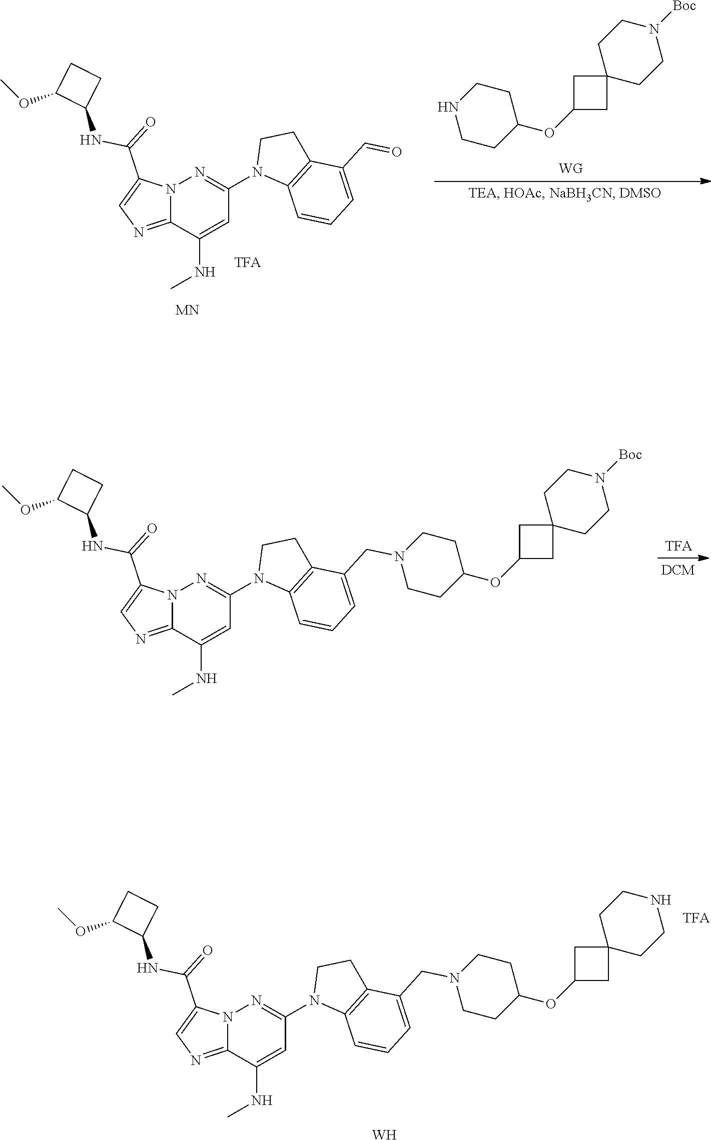

Step 1—Tert-butyl 2-[(1-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}piperidin-4-yl)oxy]-7-azaspiro[3.5]nonane-7-carboxylate. To a stirred solution of 6-(4-formyl-2,3-dihydroindol-1-yl)-N-[(1R,2R)-2-methoxycyclobutyl]-8-(methylamino)imidazo[1,2-b]pyridazine-3-carboxamide trifluoroacetate (247.10 mg, 0.462 mmol, Intermediate MN) in DMSO (5 mL) was added Et₃N (0.19 mL, 1.39 mmol) at rt under nitrogen atmosphere. To the above mixture were added tert-butyl 2-(piperidin-4-yloxy)-7-azaspiro[3.5]nonane-7-carboxylate (150 mg, 0.462 mmol, Intermediate WG) and AcOH (0.11 mL, 2.31 mmol) at rt. The resulting mixture was stirred for an additional 1 h at 50° C. The mixture was allowed to cool down to rt. To the above mixture was added sodium cyanoboranuide (58.10 mg, 0.924 mmol) at rt. Then the mixture was stirred for an additional 1 h at 50° C. On completion, the mixture was cooled to rt and concentrated under vacuum. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 75%-95% B in 25 min; Flow rate: 60 mL/min; Detector: 220/254 nm; desired fractions were collected at 93% B and concentrated under reduced pressure) to afford the title compound (260 mg, 77% yield) as a white solid. LC/MS (ESI, m/z): [(M+H)]⁺=729.3.

Step 2—6-{4-[(4-{7-Azaspiro[3.5]nonan-2-yloxy}piperidin-1-yl)methyl]-2,3-dihydroindol-1-yl}-N-[(1R,2R)-2-methoxycyclobutyl]-8-(methylamino)imidazo[1,2-b]pyridazine-3-carboxamide trifluoroacetate. To a stirred solution of tert-butyl 2-[(1-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}piperidin-4-yl)oxy]-7-azaspiro[3.5]nonane-7-carboxylate (260 mg, 0.357 mmol) in DCM (3 mL) was added TFA (1.5 mL) at rt under nitrogen atmosphere. The resulting mixture was stirred at rt for 1 h. On completion, the mixture was concentrated under reduced pressure. The residue was purified by trituration with Et₂O (5 mL) to give the title compound (300 mg) as a white solid. LC/MS (ESI, m/z): [(M+H)]⁺=629.2.

### Benzyl (4'S)-4-{[1-(tert-butoxycarbonyl)piperidin-4-yl]methyl}-3′,3′-difluoro-[1,4′-bipiperidine]-1′-carboxylate (Intermediate WI) and benzyl (4′R)-4-{[1-(tert-butoxycarbonyl)piperidin-4-yl]methyl}-3′,3′-difluoro-[1,4′-bipiperidine]-1′-carboxylate (Intermediate WJ)

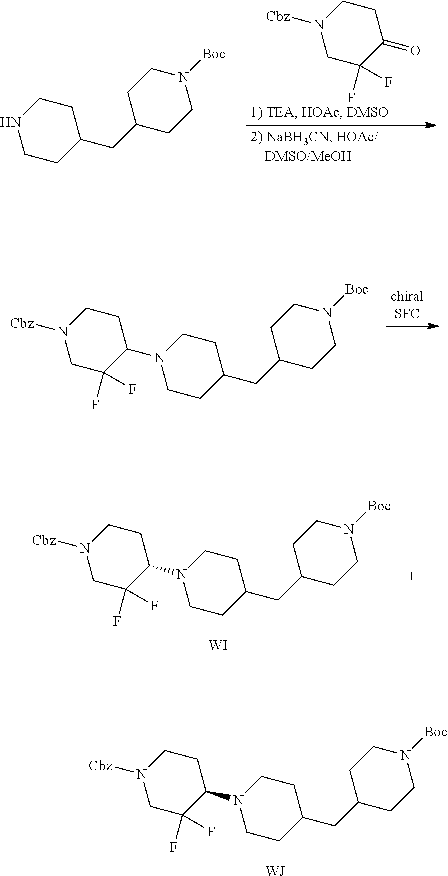

Step 1—Benzyl 4-{[1-(tert-butoxycarbonyl)piperidin-4-yl]methyl}-3′,3′-difluoro-[1,4′-bipiperidine]-1′-carboxylate. To a stirred mixture of tert-butyl 4-(piperidin-4-ylmethyl)piperidine-1-carboxylate (10 g, 35 mmol, synthesized via Steps 1-2 of Intermediate ND) in DMSO (100 mL) was added TEA (9.84 mL, 70.8 mmol) at rt under nitrogen atmosphere. The mixture was basified to pH 8. To the above mixture were added benzyl 3,3-difluoro-4-oxopiperidine-1-carboxylate (14.30 g, 53.11 mmol, CAS #1283720-88-6) and HOAc (6.09 mL, 106.221 mmol) at rt under nitrogen atmosphere. The resulting mixture was stirred at 100° C. for 2 h. Then the mixture was cooled to rt and MeOH (40 mL), HOAc (4 mL) and NaBH₃CN (2.90 g, 46.1 mmol) was added at rt under nitrogen atmosphere. The resulting mixture was stirred at 50° C. for an additional 16 h. On completion, the mixture was cooled to rt and concentrated under vacuum. The reaction mixture was purified by reverse phase flash chromatography (Column: WelFlash™ C18-I, 20-40 μm, 330 g; Eluent A: Water (plus 10 mmol/L NH₄HCO₃); Eluent B: ACN; Gradient: 95%-55% B in 25 min; Flow rate: 80 mL/min; Detector: 220/254 nm; desired fractions were collected at 95% B and concentrated under reduced pressure) to afford the title compound (6.0 g, 73% yield) as a yellow oil. LC/MS (ESI, m/z): [(M+H)]⁺=536.3.

Step 2—Benzyl (4'S)-4-{[1-(tert-butoxycarbonyl)piperidin-4-yl]methyl}-3′,3′-difluoro-[1,4′-bipiperidine]-1′-carboxylate and benzyl (4′R)-4-{[1-(tert-butoxycarbonyl)piperidin-4-yl]methyl}-3′,3′-difluoro-[1,4′-bipiperidine]-1′-carboxylate. Benzyl 4-{[1-(tert-butoxycarbonyl)piperidin-4-yl]methyl}-3′,3′-difluoro-[1,4′-bipiperidine]-1′-carboxylate (6 g, 11.201 mmol) was separated by Prep-chiral-SFC under the following conditions: Column: CHIRALPAK IG, 3*25 cm, 5 μm; Mobile Phase A: 1% 2MNH₃-MEOH, Mobile Phase B: ACN: MEOH=1: 1; Flow rate: 100 mL/min; Gradient: isocratic 45% B; Back Pressure(bar): 100; Wave Length: 220 nm; RT1(min): 3.25; RT2(min): 5.17; Sample Solvent: MEOH; Injection Volume: 2 mL; Number Of Runs: 11. The fractions of faster eluting peak was collected and concentrated under vacuum to afford benzyl (4′R)-4-{[1-(tert-butoxycarbonyl)piperidin-4-yl]methyl}-3′,3′-difluoro-[1,4′-bipiperidine]-1-carboxylate (3.0 g, 50%). The fractions of slower eluting peak was collected and concentrated under vacuum to afford benzyl (4'S)-4-{[1-(tert-butoxycarbonyl)piperidin-4-yl]methyl}-3′,3′-difluoro-[1,4′-bipiperidine]-1′-carboxylate (2.9 g, 48%). LC/MS (ESI, m/z): [(M+H)]-=536.3. The absolute configuration of the stereoisomers was arbitrarily assigned.

### Tert-butyl 4-{[(4′R)-3′,3′-difluoro-[1,4′-bipiperidin]-4-yl]methyl}piperidine-1-carboxylate (Intermediate WK)

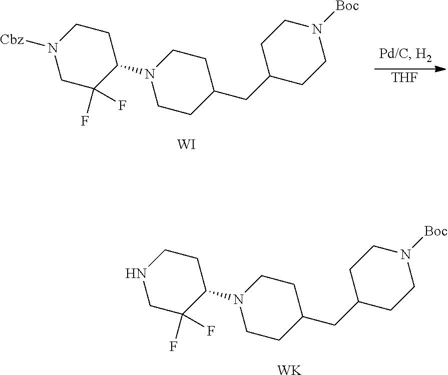

To a stirred solution of benzyl (4′R)-4-{[1-(tert-butoxycarbonyl)piperidin-4-yl]methyl}-3′,3′-difluoro-[1,4′-bipiperidine]-1′-carboxylate (2.9 g, 5.414 mmol, Intermediate WI) in THF (30 mL) was added Pd/C (576.13 mg, 10 wt %) at rt under nitrogen atmosphere. The reaction system was degassed under vacuum and purged with H₂ several times, then it was hydrogenated under H₂ balloon (1 atm) at rt for 1 h. After completion of the reaction, the resulting mixture was filtered, the filter cake was washed with THF (3×100 mL). The filtrate was concentrated under reduced pressure to give the title compound (2.0 g, 92% yield) as a grey solid. LC/MS (ESIL m/z): [(M+H)]⁺=402.1.

### 6-(4-{[(4'S)-3′,3′-difluoro-4-(piperidin-4-ylmethyl)-[1,4′-bipiperidin]-1′-yl]methyl}-2,3-dihydroindol-1-yl)-N-[(1R,2R)-2-methoxycyclobutyl]-8-(methylamino)imidazo[1,2-b]pyridazine-3-carboxamide (Intermediate WL)

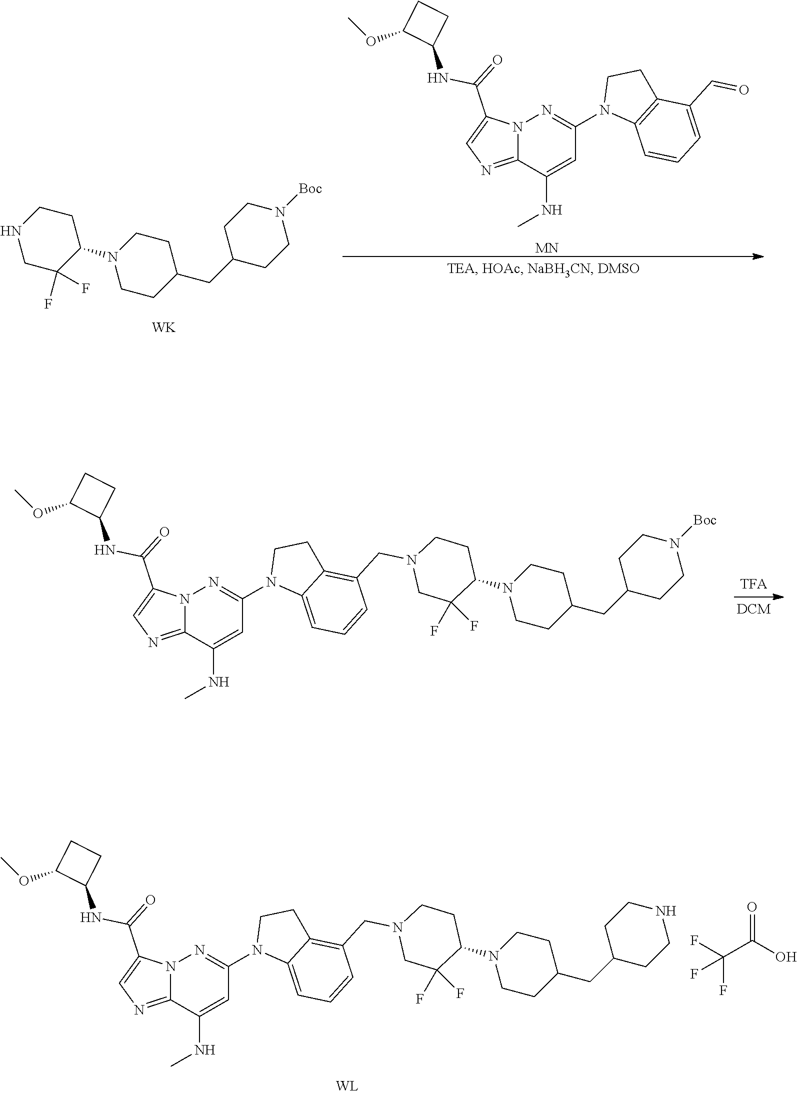

Step 1—Tert-butyl 4-{[(4'S)-3′,3‘-difluoro-1’-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}-[1,4′-bipiperidin]-4-yl]methyl}piperidine-1-carboxylate. To a stirred solution of tert-butyl 4-{[(4'S)-3′,3′-difluoro-[1,4′-bipiperidin]-4-yl]methyl}piperidine-1-carboxylate (1.7 g, 4.2 mmol, Intermediate WK) in DMSO (20 mL) was added TEA (1.77 mL, 12.7 mmol) at rt under nitrogen atmosphere. The resulting mixture was stirred at rt for 5 min. To the above mixture were added 6-(4-formyl-2,3-dihydroindol-1-yl)-N-[(1R,2R)-2-methoxycyclobutyl]-8-(methylamino)imidazo[1,2-b]pyridazine-3-carboxamide (1.78 g, 4.23 mmol, Intermediate MN) and HOAc (1.27 mL, 21.170 mmol) in turns at rt. The resulting mixture was stirred at 50° C. for an additional 30 min. The mixture was allowed to cool down to rt. To the above mixture was added NaBH₃CN (1.33 g, 21.170 mmol) at rt. The resulting mixture was stirred at 50° C. for an additional 2 h. After completion of the reaction, the reaction mixture was cooled to rt and purified directly by reverse phase flash chromatography (Column: WelFlash™ C18-I, 20-40 μm, 330 g; Eluent A: Water (plus 10 mmol/L NH₄HCO₃); Eluent B: ACN; Gradient: 25%-55% B in 25 min; Flow rate: 80 mL/min; Detector: 220/254 nm; desired fractions were collected at 50% B and concentrated under reduced pressure) to afford the title compound (1.4 g, 41% yield) as a yellow solid. LC/MS (ESI, m/z): [(M+H)]⁺=806.4.

Step 2—6-(4-{[(4'S)-3′,3′-Difluoro-4-(piperidin-4-ylmethyl)-[1,4′-bipiperidin]-1′-yl]methyl}-2,3-dihydroindol-1-yl)-N-[(1R,2R)-2-methoxycyclobutyl]-8-(methylamino)imidazo[1,2-b]pyridazine-3-carboxamide trifluoroacetate. To a stirred solution of tert-butyl 4-{[(4'S)-3′,3‘-difluoro-1’-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}-[1,4′-bipiperidin]-4-yl]methyl}piperidine-1-carboxylate (1.4 g, 1.7 mmol) in DCM (30 mL) was added trifluoroacetic acid (10 mL) at rt under nitrogen atmosphere. The resulting mixture was stirred at rt for 1 h under nitrogen atmosphere. Upon completion, the mixture was concentrated under reduced pressure. The residue was purified by trituration with Et₂O (50 mL) to afford the title compound (1.4 g) as a yellow solid. LC/MS (ESI, m/z): [(M+H)]⁺=706.4.

### Tert-butyl (R)-4-((3′,3′-difluoro-[1,4′-bipiperidin]-4-yl)methyl)piperidine-1-carboxylate (Intermediate WM)

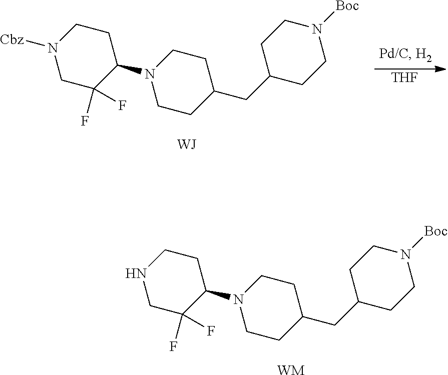

To a stirred solution of benzyl (R)-4-((1-(tert-butoxycarbonyl)piperidin-4-yl)methyl)-3′,3′-difluoro-[1,4′-bipiperidine]-1′-carboxylate (2.90 g, 5.41 mmol, Intermediate WJ) in THF (30 mL) was added Pd/C (576 mg, 5.41 mmol) at rt under nitrogen atmosphere. The reaction system was degassed under vacuum and purged with H₂ several times, then it was hydrogenated under H₂ balloon (˜1 atm) at 25° C. for 4 h. After completion of the reaction, Pd/C was filtered off through celite and the filter cake was washed with THF (3×100 mL). The filtrate was concentrated under reduced pressure to give the title compound (2.0 g, 92% yield) as a grey solid. LC/MS (ESI, m/z): [(M+H)]⁺=402.1.

### 6-(4-(((R)-3′,3′-difluoro-4-(piperidin-4-ylmethyl)-[1,4′-bipiperidin]-1′-yl)methyl)indolin-1-yl)-N-((1R,2R)-2-methoxycyclobutyl)-8-(methylamino)imidazo[1,2-b]pyridazine-3-carboxamide (Intermediate WN)

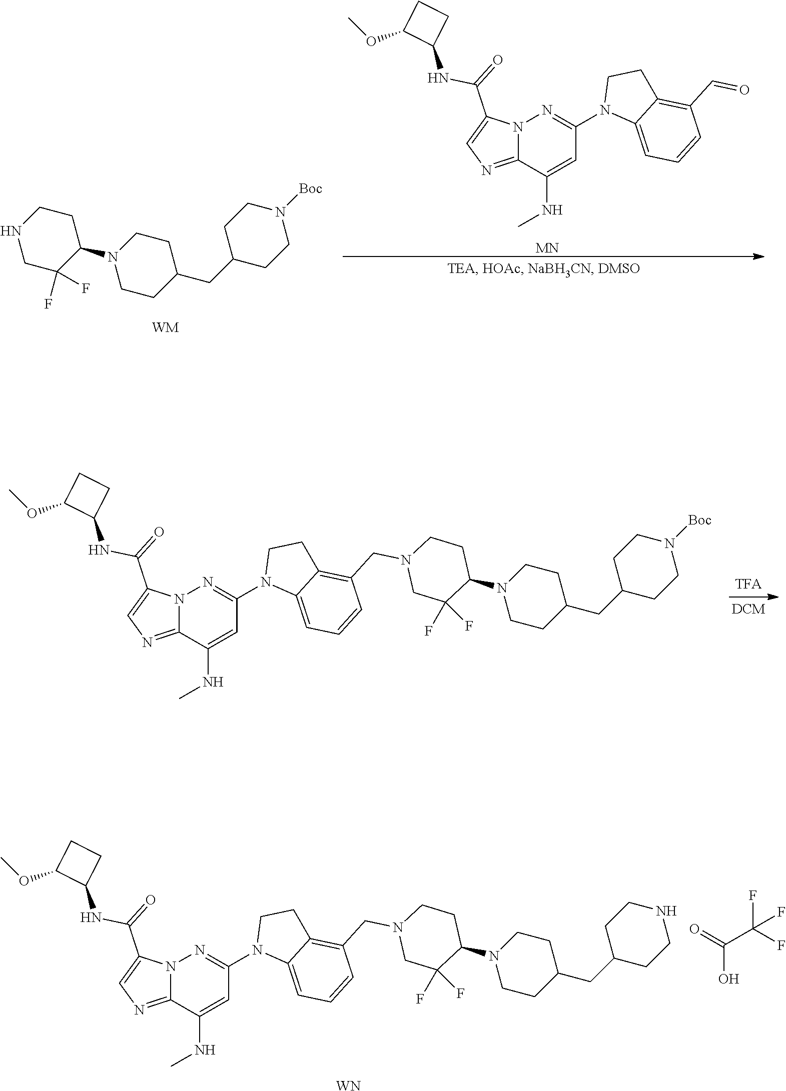

Step 1—Tert-butyl 4-(((R)-3′,3′-difluoro-1′-((1-(3-(((1R,2R)-2-methoxycyclobutyl)carbamoyl)-8-(methylamino)imidazo[1,2-b]pyridazin-6-yl)indolin-4-yl)methyl)-[1,4′-bipiperidin]-4-yl)methyl)piperidine-1-carboxylate. To a stirred solution of tert-butyl (R)-4-((3′,3′-difluoro-[1,4′-bipiperidin]-4-yl)methyl)piperidine-1-carboxylate (2.00 g, 4.98 mmol, Intermediate WM) in DMSO (20 mL) was added TEA (1.01 g, 9.96 mmol) at 25° C. The resulting mixture was stirred for 5 min at rt. The mixture was then basified to pH 8. To the above mixture was added 6-(4-formylindolin-1-yl)-N-((1R,2R)-2-methoxycyclobutyl)-8-(methylamino)imidazo[1,2-b]pyridazine-3-carboxamide (2.09 g, 4.98 mmol, Intermediate MN) and HOAc (897 mg, 14.94 mmol) at rt. The resulting mixture was stirred for an additional 30 min at rt. To the above mixture was added NaBH₃CN (939 mg, 14.943 mmol) in portions over 5 min at rt. The resulting mixture was stirred for 2 h at 50° C. under nitrogen atmosphere. After completion of the reaction, the reaction mixture was cooled to rt and purified directly by reverse phase flash chromatography (Column: WelFlash™ C18-I, 20-40 μm, 330 g; Eluent A: Water (plus 5 mmol/L NH₄HCO₃); Eluent B: ACN; Gradient: 75%-95% B in 35 min; Flow rate: 80 mL/min; Detector: 220/254 nm; desired fractions were collected at 95% B and concentrated under reduced pressure) to afford the title compound (1.80 g, 45% yield) as a white solid. LC/MS (ESI, m/z): [(M+H)]⁺=806.5.

Step 2—6-(4-(((R)-3′,3′-difluoro-4-(piperidin-4-ylmethyl)-[1,4′-bipiperidin]-1′-yl)methyl)indolin-1-yl)-N-((1R,2R)-2-methoxycyclobutyl)-8-(methylamino)imidazo[1,2-b]pyridazine-3-carboxamide trifluoroacetate. To a stirred solution of tert-butyl 4-(((R)-3′,3‘-difluoro-1’-((1-(3-(((1R,2R)-2-methoxycyclobutyl)carbamoyl)-8-(methylamino)imidazo[1,2-b]pyridazin-6-yl)indolin-4-yl)methyl)-[1,4′-bipiperidin]-4-yl)methyl)piperidine-1-carboxylate (1.20 g, 1.49 mmol) in DCM (12 mL) was added trifluoroacetic acid (4 mL) in portions at rt under nitrogen atmosphere. The resulting mixture was stirred for 1 h at rt under nitrogen atmosphere. On completion, the mixture was concentrated under vacuum. The residue was purified by trituration with Et₂O (60 mL) to give the title compound (1.70 g) as a white solid. LC/MS (ESI, m/z): [(M+H)]⁺=706.4.

### 1-(2,3-Dihydro-1H-indol-4-yl)pyrazole-4-carbaldehyde (Intermediate WO)

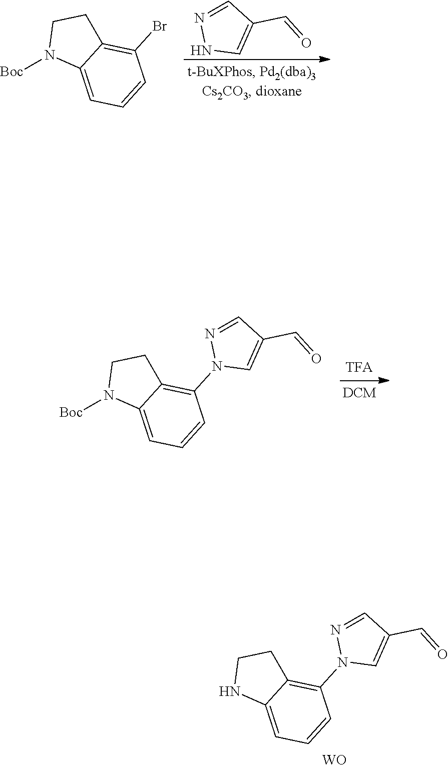

Step 1—Tert-butyl 4-(4-formvlpyrazol-1-yl)-2,3-dihydroindole-1-carboxylate. To a stirred mixture of tert-butyl 4-bromo-2,3-dihydroindole-1-carboxylate (3.00 g, 10.1 mmol), Cs₂CO₃ (9.83 g, 30.18 mmol) and 1H-pyrazole-4-carbaldehyde (2.42 g, 25.2 mmol) in 1,4-dioxane (30 mL) were added t-BuXPhos (854 mg, 2.01 mmol) and Pd₂(dba)₃ (921 mg, 1.01 mmol) at rt under nitrogen atmosphere. The resulting mixture was stirred at 100° C. for 16 h under nitrogen atmosphere. On completion, the mixture was cooled to rt and concentrated under reduced pressure. The residue was purified by silica gel column chromatography, eluted with CH₂Cl₂/EA (1:1), to afford the title compound (500 mg, 16% yield) as a light yellow oil. LC/MS (ESI, m/z): [(M+H)]⁺=314.1.

Step 2—1-(2,3-Dihydro-1H-indol-4-yl)pyrazole-4-carbaldehyde. To a stirred mixture of tert-butyl 4-(4-formylpyrazol-1-yl)-2,3-dihydroindole-1-carboxylate (500 mg, 1.60 mmol) in DCM (6 mL) was added trifluoroacetic acid (2 mL) at rt. The resulting mixture was stirred at rt for 1 h. On completion, the mixture was basified to pH 8 with saturated Na₂CO₃(aq.). The resulting mixture was extracted with CH₂C12 (3×50 mL). The combined organic layers were washed with brine (1×100 mL), and dried over anhydrous Na₂SO₄. After filtration, the filtrate was concentrated under reduced pressure to give the title compound (500 mg) as a yellow solid. LC/MS (ESI, m/z): [(M+H)]⁺=214.1.

### 6-(4-(4-formyl-1H-pyrazol-1-yl)indolin-1-yl)-N-((1R,2R)-2-methoxycyclobutyl)-8-(methylamino)imidazo[1,2-b]pyridazine-3-carboxamide (Intermediate WP)

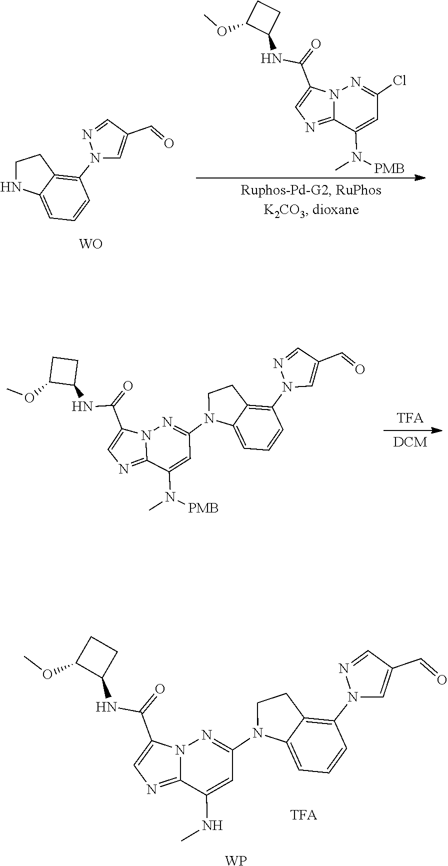

Step 1—Tert-butyl N-{6-[4-(4-formylpyrazol-1-yl)-2,3-dihydroindol-1-yl]-3-{[(1R,2R)-2-methoxycyclobutyl]carbamoyl}imidazo[1,2-b]pyridazin-8-yl}-N-methylcarbamate. To a stirred mixture of 1-(2,3-dihydro-1H-indol-4-yl)pyrazole-4-carbaldehyde (370 mg, 1.74 mmol, Intermediate WO), K₂CO₃ (719 mg, 5.20 mmol) and tert-butyl N-(6-chloro-3-{[(1R,2R)-2-methoxycyclobutyl]carbamoyl}imidazo[1,2-b]pyridazin-8-yl)-N-methylcarbamate (711 mg, 1.74 mmol, Intermediate VX) in 1,4-dioxane (10 mL) were added RuPhos (162 mg, 0.347 mmol) and RuPhos Pd G2 (270 mg, 0.348 mmol) at rt under nitrogen atmosphere. The resulting mixture was stirred at 100° C. for 1 h under nitrogen atmosphere. On completion, the mixture was cooled to rt and concentrated under reduced pressure. The residue was purified by silica gel column chromatography, eluted with CH₂C2/EA (1:1), to afford the title compound (500 mg, 49% yield) as a white solid. LC/MS (ESI, m/z): [(M+H)]⁺=607.3.

Step 2—6-(4-(4-Formyl-1H-pyrazol-1-yl)indolin-1-yl)-N-((1R,2R)-2-methoxycyclobutyl)-8-(methylamino)imidazo[1,2-b]pyridazine-3-carboxamide trifluoroacetate. To a stirred solution of tert-butyl N-{6-[4-(4-formylpyrazol-1-yl)-2,3-dihydroindol-1-yl]-3-{[(1R,2R)-2-methoxycyclobutyl]carbamoyl}imidazo[1,2-b]pyridazin-8-yl}-N-methylcarbamate (500 mg, 0.852 mmol) in DCM (6 mL) was added trifluoroacetic acid (2 mL) at rt. The resulting mixture was stirred at rt for 1 h. Upon completion, the mixture was concentrated under reduced pressure. The residue was purified by trituration with Et₂O (10 mL) to afford the title compound (600 mg, TFA salt) as a yellow solid. LC/MS (ESI, m/z): [(M+H)]⁺=487.3.

### 6-(4-(4-((1,1-Difluoro-3,9-diazaspiro[5.5]undecan-3-yl)methyl)-1H-pyrazol-1-yl)indolin-1-yl)-N-((1R,2R)-2-methoxycyclobutyl)-8-(methylamino)imidazo[1,2-b]pyridazine-3-carboxamide (Intermediate WQ)

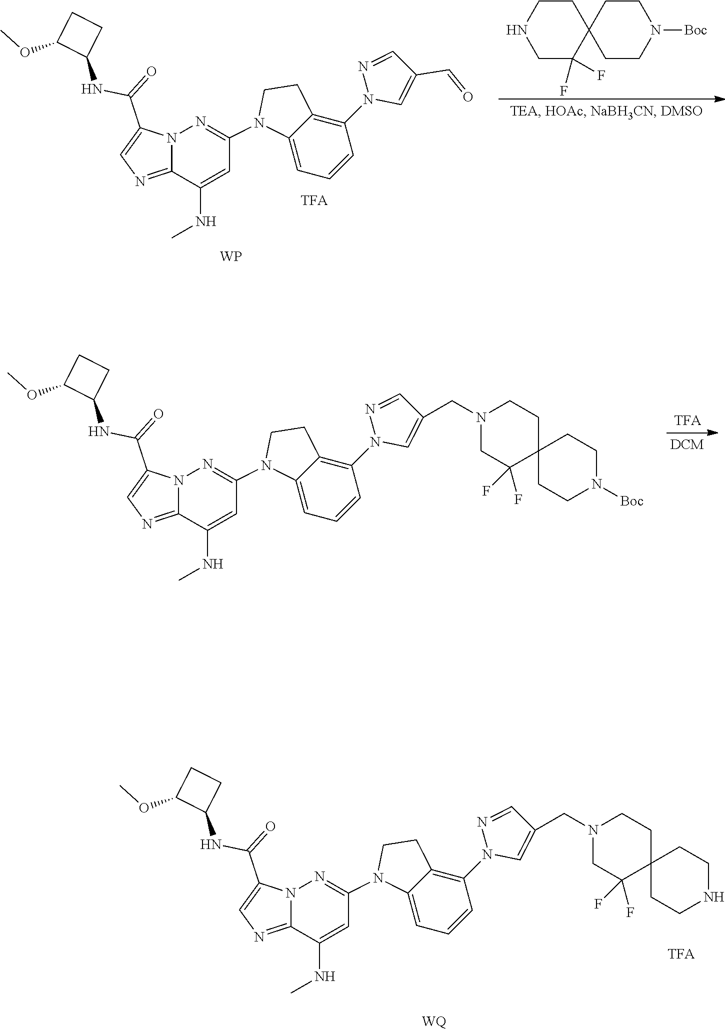

Step 1—Tert-butyl 7,7-difluoro-9-({l-[1-(3-{[(1R,2R)-2-methoxycyclobutyl]carbamoyl}-8-(methylamino)imidazo[1,2-b]pyridazin-6-yl)-2,3-dihydroindol-4-yl]pyrazol-4-yl}methyl)-3,9-diazaspiro[5.5]undecane-3-carboxylate. To a stirred mixture of 6-(4-(4-formyl-1H-pyrazol-1-yl)indolin-1-yl)-N-((1R,2R)-2-methoxycyclobutyl)-8-(methylamino)imidazo[1,2-b]pyridazine-3-carboxamide trifluoroacetate (300 mg, 0.500 mmol, Intermediate WP) and Et₃N (0.25 mL, 1.50 mmol) in DMSO (5 mL) were added tert-butyl 7,7-difluoro-3,9-diazaspiro[5.5]undecane-3-carboxylate (181 mg, 0.623 mmol) and AcOH (0.25 mL, 3.00 mmol) at rt. The resulting mixture was stirred at 50° C. for 30 min under nitrogen atmosphere. To the above mixture was added NaBH₃CN (120 mg, 1.910 mmol) and the mixture was stirred at 50° C. for an additional 1 h. On completion, the mixture was cooled to rt and purified directly by reverse phase flash chromatography (Column: WelFlash™ C18-I, 20-40 μm, 120 g; Eluent A: Water (plus 10 mmol/L NH₄HCO₃); Eluent B: ACN; Gradient: 55%-75% B in 15 min; Flow rate: 60 mL/min; Detector: 220/254 nm; desired fractions were collected at 65% B and concentrated under reduced pressure) to afford the title compound (200 mg, 43% yield) as a white solid. LC/MS (ESI, m/z): [(M+H)]⁺=761.5.

Step 2—6-(4-(4-((1,1-Difluoro-3,9-diazaspiro[5.5]undecan-3-yl)methyl)-1H-pyrazol-1-yl)indolin-1-yl)-N-((1R,2R)-2-methoxycyclobutyl)-8-(methylamino)imidazo[1,2-b]pyridazine-3-carboxamide trifluoroacetate. To a stirred mixture of tert-butyl 7,7-difluoro-9-({1-[1-(3-{[(1R,2R)-2-methoxycyclobutyl]carbamoyl}-8-(methylamino)imidazo[1,2-b]pyridazin-6-yl)-2,3-dihydroindol-4-yl]pyrazol-4-yl}methyl)-3,9-diazaspiro[5.5]undecane-3-carboxylate (200 mg, 0.263 mmol) in DCM (3 mL) was added trifluoroacetic acid (1 mL) at rt. The resulting mixture was stirred at rt for 1 h. On completion, the mixture was concentrated under reduced pressure. The residue was purified by trituration with Et₂O (10 mL) to afford the title compound (200 mg, TFA salt) as a light yellow solid. LC/MS (ESI, m/z): [(M+H)]=661.3.

### 1-(2-Chloro-5-(9-((3,3-difluoropiperidin-4-yl)methyl)-3,9-diazaspiro[5.5]undecane-3-carbonyl)-3-methylphenyl)dihydropyrimidine-2,4(1H,3H)-dione (Intermediate WR)

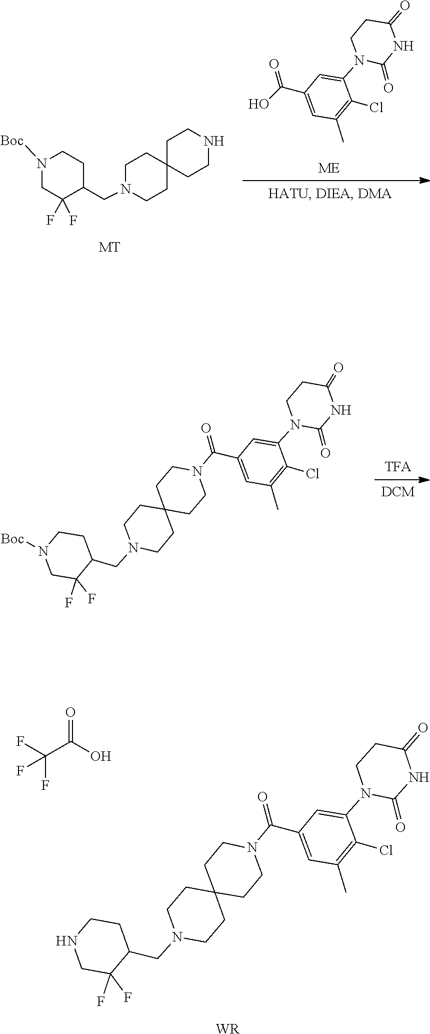

Step 1—Tert-butyl 4-((9-(4-chloro-3-(2,4-dioxotetrahydropyrimidin-1(2H)-yl)-5-methylbenzoyl)-3,9-diazaspiro[5.5]undecan-3-yl)methyl)-3,3-difluoropiperidine-1-carboxylate. To a stirred solution of tert-butyl 4-((3,9-diazaspiro[5.5]undecan-3-yl)methyl)-3,3-difluoropiperidine-1-carboxylate (200 mg, 0.510 mmol, Intermediate MT) and 4-chloro-3-(2,4-dioxo-1,3-diazinan-1-yl)-5-methylbenzoic acid (145 mg, 0.514 mmol, Intermediate ME) in DMA (4 mL) were added HATU (294 mg, 0.771 mmol) and DIEA (200 mg, 1.54 4 mmol) in turns at rt under nitrogen atmosphere. The resulting mixture was stirred for 1 h at rt under nitrogen atmosphere. After completion of the reaction, the reaction mixture was purified directly by reverse phase flash chromatography (Column: WelFlash™ C18-I, 20-40 μm, 120 g; Eluent A: Water (10 mmol/L NH₄HCO₃); Eluent B: ACN; Gradient: 30%-60% B in 40 min; Flow rate: 60 mL/min; Detector: 220/254 nm; desired fractions were collected at 50% B and concentrated under reduced pressure) to afford the title compound (70 mg, 20% yield) as a yellow solid. LC/MS (ESI, m/z): [(M+1)]⁺=652.2.

Step 2—1-(2-Chloro-5-(9-((3,3-difluoropiperidin-4-yl)methyl)-3,9-diazaspiro[5.5]undecane-3-carbonyl)-3-methylphenyl)dihydropyrimidine-2,4(1H,3H)-dione trifluoroacetate. To a stirred solution of tert-butyl 4-((9-(4-chloro-3-(2,4-dioxotetrahydropyrimidin-1(2H)-yl)-5-methylbenzoyl)-3,9-diazaspiro[5.5]undecan-3-yl)methyl)-3,3-difluoropiperidine-1-carboxylate (70 mg, 0.101 mmol) in DCM (3 mL) was added TFA (1 mL) dropwise at rt under nitrogen atmosphere. The resulting mixture was stirred for 1 h at rt under nitrogen atmosphere. The resulting mixture was concentrated under reduced pressure. The residue was purified by trituration with Et₂O (10 mL) to give the title compound (59 mg, 99% yield) as a yellow solid. LC/MS (ESI, m/z): [(M+1)]f=552.4.

### 6-[4-({5,5-Difluoro-2,7-diazaspiro[3.5]nonan-2-yl}methyl)-2,3-dihydroindol-1-yl]-N-[(1R,2R)-2-methoxycyclobutyl]-8-(methylamino)imidazo[1,2-b]pyridazine-3-carboxamide (Intermediate WS)

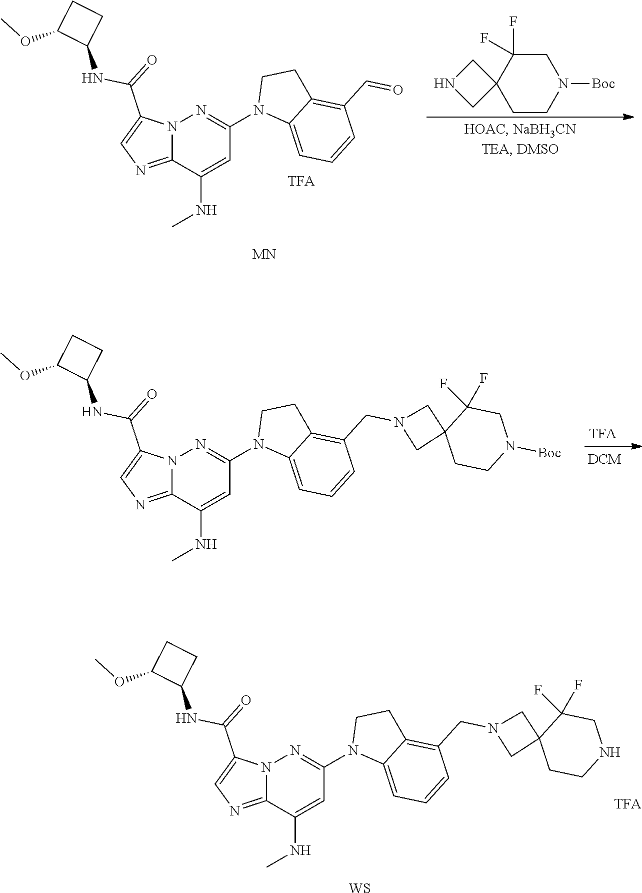

Step 1—Tert-butyl 5,5-difluoro-2-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}-2,7-diazaspiro[3.5]nonane-7-carboxylate. To a stirred mixture of 6-(4-formyl-2,3-dihydroindol-1-yl)-N-[(1R,2R)-2-methoxycyclobutyl]-8-(methylamino)imidazo[1,2-b]pyridazine-3-carboxamide trifluoroacetate (300 mg, 0.579 mmol, Intermediate MN) and tert-butyl 5,5-difluoro-2,7-diazaspiro[3.5]nonane-7-carboxylate (151.77 mg, 0.579 mmol) in DMSO (5 mL) were added Et₃N (0.24 mL, 1.737 mmol) and AcOH (0.10 mL, 1.74 mmol) at rt under nitrogen atmosphere. The resulting mixture was stirred at 50° C. for 0.5 h under nitrogen atmosphere. To the above mixture was added sodium cyanoboranuide (181.79 mg, 2.895 mmol) at rt. The resulting mixture was stirred at 50° C. for an additional 16 h. On completion, the mixture was cooled to rt and concentrated under reduced pressure. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 45%-75% B in 20 min; Flow rate: 80 mL/min; Detector: 220/254 nm; desired fractions were collected at 69% B and concentrated under reduced pressure) to afford the title compound (260 mg, 67% yield) as an off-white solid. LC/MS (ESI, m/z): [(M+H)]⁺=667.3.

Step 2—6-[4-({5,5-Difluoro-2,7-diazaspiro[3.5]nonan-2-yl}methyl)-2,3-dihydroindol-1-yl]-N-[(1R,2R)-2-methoxycyclobutyl]-8-(methylamino)imidazo[1,2-b]pyridazine-3-carboxamide trifluoroacetate. To a stirred solution of tert-butyl 5,5-difluoro-2-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}-2,7-diazaspiro[3.5]nonane-7-carboxylate (260 mg, 0.390 mmol) in DCM (4 mL) was added TFA (2 mL) at rt under nitrogen atmosphere. The resulting mixture was stirred at rt for 1 h under nitrogen atmosphere. On completion, the mixture was concentrated under reduced pressure. The residue was purified by trituration with Et₂O (10 mL) to give the title compound (250 mg) as a white solid. LC/MS (ESI, m/z): [(M+H)]⁺=567.3.

### 6-(4-{[5,5-Difluoro-7-(piperidin-4-yl)-2,7-diazaspiro[3.5]nonan-2-yl]methyl}-2,3-dihydroindol-1-yl)-N-[(1R,2R)-2-methoxycyclobutyl]-8-(methylamino)imidazo[1,2-b]pyridazine-3-carboxamide (Intermediate WT)

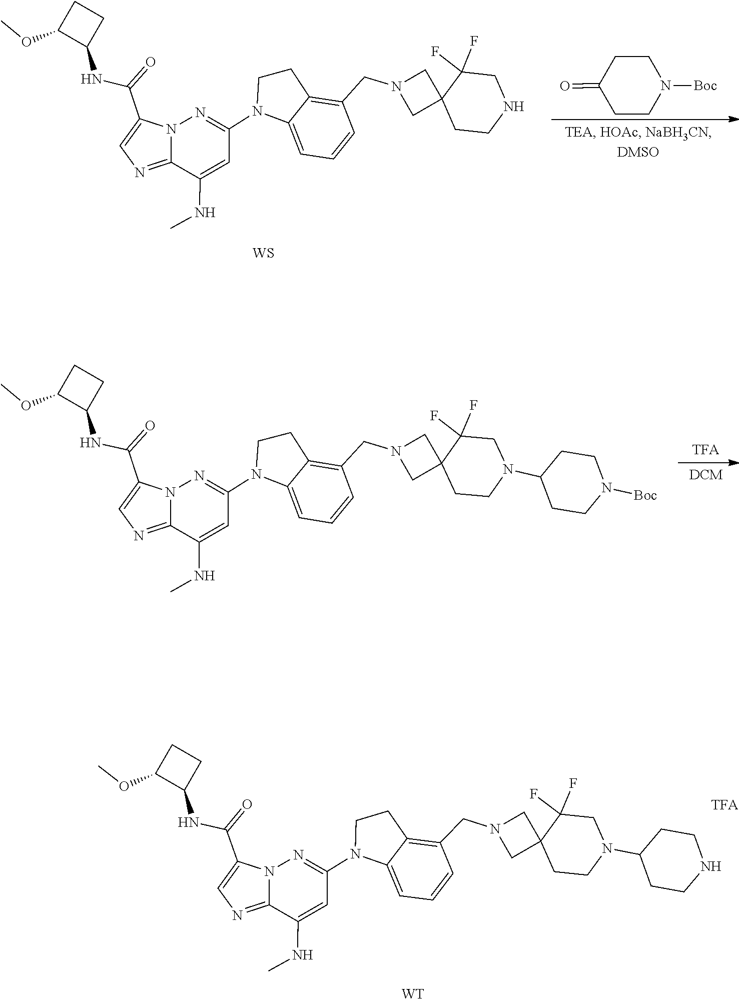 [SMILES: CNc1cc(N2CCc3c(CN4CC5(CCNCC5(F)F)C4)cccc32)nn2c(C(=O)N[C@@H]3CC[C@H]3OC)cnc12 ; CC(C)(C)OC(=O)N1CCC(=O)CC1 ; "WS" ; CNc1cc(N2CCc3c(CN4CC5(CCN(C6CCN(C(=O)OC(C)(C)C)CC6)CC5(F)F)C4)cccc32)nn2c(C(=O)N[C@@H]3CC[C@H]3OC)cnc12 ; CNc1cc(N2CCc3c(CN4CC5(CCN(C6CCNCC6)CC5(F)F)C4)cccc32)nn2c(C(=O)N[C@@H]3CC[C@H]3OC)cnc12 ; "WT"]

Step 1—Tert-butyl 4-(5,5-difluoro-2-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}-2,7-diazaspiro[3.5]nonan-7-yl)piperidine-1-carboxylate. To a stirred solution of 6-[4-({5,5-difluoro-2,7-diazaspiro[3.5]nonan-2-yl}methyl)-2,3-dihydroindol-1-yl]-N-[(1R,2R)-2-methoxycyclobutyl]-8-(methylamino)imidazo[1,2-b]pyridazine-3-carboxamide trifluoroacetate (250 mg, 0.376 mmol, Intermediate WS) in DMSO (5 mL) was added Et₃N (0.16 mL, 1.13 mmol) at rt under nitrogen atmosphere. To the above mixture were added tert-butyl 4-oxopiperidine-1-carboxylate (89.93 mg, 0.451 mmol) and AcOH (0.11 mL, 1.88 mmol) at rt. The resulting mixture was stirred at 50° C. for 1 h. To the above mixture was added sodium cyanoboranuide (118.18 mg, 1.880 mmol) at rt. The resulting mixture was stirred at 50° C. for an additional 16 h. Upon completion, the mixture was cooled to rt and concentrated under reduced pressure. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 50%-80% B in 20 min; Flow rate: 60 mL/min; Detector: 220/254 nm; desired fractions were collected at 75% B and concentrated under reduced pressure) to afford the title compound (150 mg, 53% yield) as a white solid. LC/MS (ESI, m/z): [(M+H)]⁺=750.4.

Step 2—6-(4-{[5,5-Difluoro-7-(piperidin-4-yl)-2,7-diazaspiro[3.5]nonan-2-yl]methyl}-2,3-dihydroindol-1-yl)-N-[(1R,2R)-2-methoxycyclobutyl]-8-(methylamino)imidazo[1,2-b]pyridazine-3-carboxamide trifluoroacetate. To a stirred solution of tert-butyl 4-(5,5-difluoro-2-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}-2,7-diazaspiro[3.5]nonan-7-yl)piperidine-1-carboxylate (150 mg, 0.200 mmol) in DCM (2 mL) was added TFA (1 mL) at rt under nitrogen atmosphere. The resulting mixture was stirred at rt for 1 h under nitrogen atmosphere. On completion, the mixture was concentrated under reduced pressure. The residue was purified by trituration with Et₂O (10 mL) to give the title compound (140 mg) as a white solid. LC/MS (ESI, m/z): [(M+H)]m=650.3.

### 6-[4-({5,5-Difluoro-2,7-diazaspiro[3.5]nonan-7-yl}methyl)-2,3-dihydroindol-1-yl]-N-[(1R,2R)-2-methoxycyclobutyl]-8-(methylamino)imidazo[1,2-b]pyridazine-3-carboxamide (Intermediate WU)

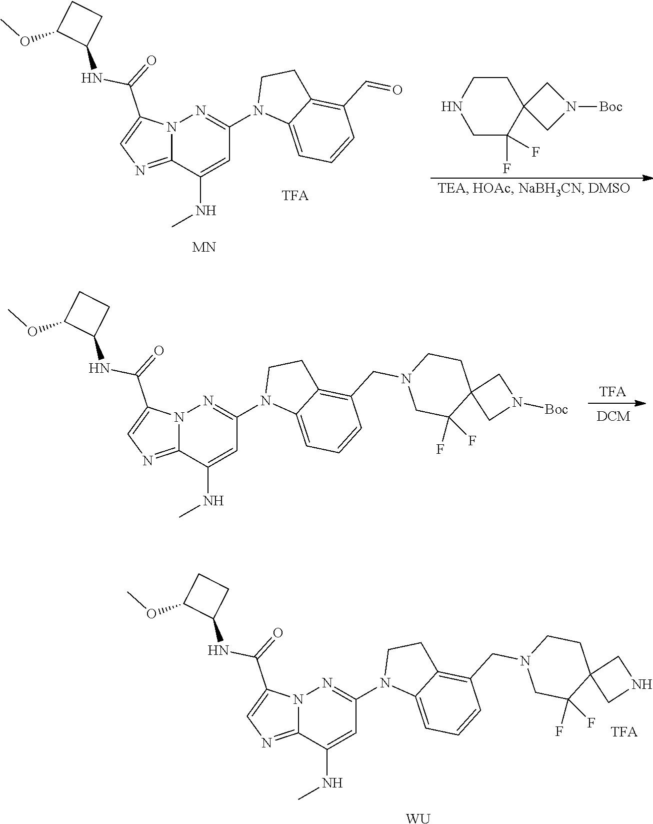

Step 1—Tert-butyl 5,5-difluoro-7-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}-2,7-diazaspiro[3.5]nonane-2-carboxylate. To a stirred solution of tert-butyl 5,5-difluoro-2,7-diazaspiro[3.5]nonane-2-carboxylate (187.15 mg, 0.713 mmol. CASH 2007920-32-1) in DMSO (6 mL) was added Et₃N (0.3 mL, 2.14 mmol) at rt under nitrogen atmosphere. The resulting mixture was stirred at rt for 5 min under nitrogen atmosphere. To the above mixture were added 6-(4-formyl-2,3-dihydroindol-1-yl)-N-[(1R,2R)-2-methoxycyclobutyl]-8-(methylamino)imidazo[1,2-b]pyridazine-3-carboxamide (370 mg, 0.713 mmol, Intermediate MN) and AcOH (0.2 mL, 3.7 mmol) at rt. The resulting mixture was stirred at 50° C. for an additional 30 min. The mixture was allowed to cool down to rt. To the above mixture was added sodium cyanoboranuide (134.51 mg, 2.139 mmol) at rt. The resulting mixture was stirred at 50° C. for an additional 2 h. On completion, the mixture was cooled to rt and concentrated under reduced pressure. The residue was purified by reverse flash chromatography (column, C18 silica gel; mobile phase A: water (10 mmol/L NH₄HCO₃), mobile phase B: ACN, 30% to 50% gradient in 30 min; detector, UV 254 nm; the fractions were collected at 45% and concentrated under reduced pressure) to afford the title compound (160 mg, 34% yield) as a white solid. LC/MS (ESI, m/z): [(M+H)]⁺=667.4.

Step 2—6-[4-({5,5-Difluoro-2,7-diazaspiro[3.5]nonan-7-yl}methyl)-2,3-dihydroindol-1-yl]-N-[(1R,2R)-2-methoxycyclobutyl]-8-(methylamino)imidazo[1,2-b]pyridazine-3-carboxamide trifluoroacetate. To a stirred solution of tert-butyl 5,5-difluoro-7-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}-2,7-diazaspiro[3.5]nonane-2-carboxylate (160 mg, 0.240 mmol) in DCM (4 mL) was added TFA (2 mL) at rt under nitrogen atmosphere. The resulting mixture was stirred for 1 h at rt under nitrogen atmosphere. On completion, the mixture was concentrated under reduced pressure. The residue was purified by trituration with ethyl ether (10 mL) to afford the title compound (150 mg) as a white solid. LC/MS (ESI, m/z): [(M+H)]⁺=567.3.

### 6-(4-{[5,5-Difluoro-2-(piperidin-4-yl)-2,7-diazaspiro[3.5]nonan-7-yl]methyl}-2,3-dihydroindol-1-yl)-N-[(1R,2R)-2-methoxycyclobutyl]-8-(methylamino)imidazo[1,2-b]pyridazine-3-carboxamide (Intermediate WV)

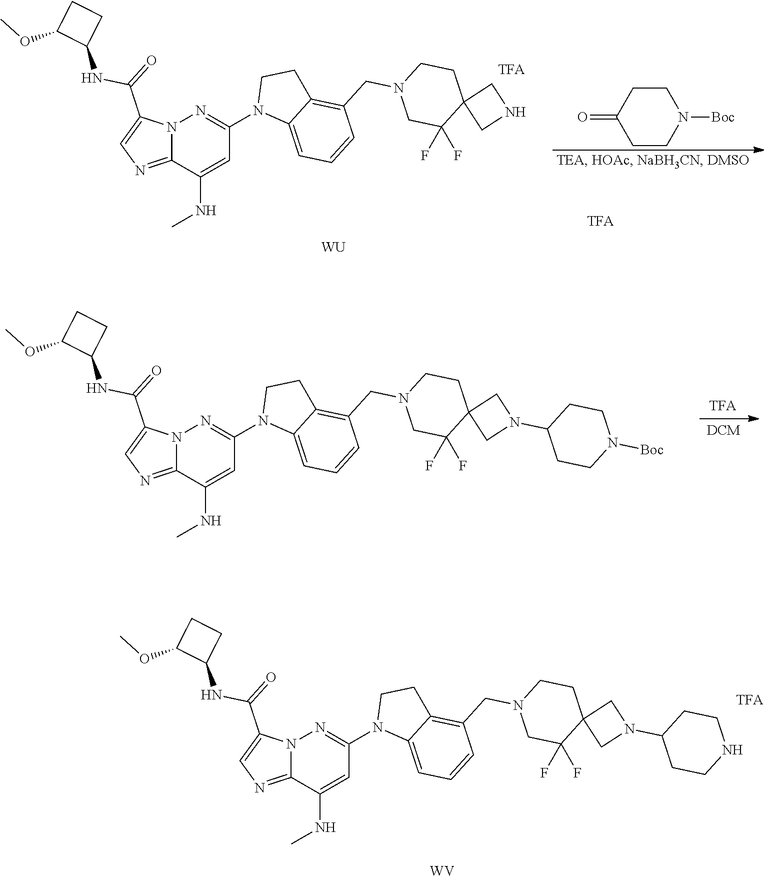 [SMILES: CNc1cc(N2CCc3c(CN4CCC5(CNC5)C(F)(F)C4)cccc32)nn2c(C(=O)N[C@@H]3CC[C@H]3OC)cnc12 ; CC(C)(C)OC(=O)N1CCC(=O)CC1 ; "WU" ; CNc1cc(N2CCc3c(CN4CCC5(CN(C6CCN(C(=O)OC(C)(C)C)CC6)C5)C(F)(F)C4)cccc32)nn2c(C(=O)N[C@@H]3CC[C@H]3OC)cnc12 ; CNc1cc(N2CCc3c(CN4CCC5(CN(C6CCNCC6)C5)C(F)(F)C4)cccc32)nn2c(C(=O)N[C@@H]3CC[C@H]3OC)cnc12 ; "WV"]

Step 1—Tert-butyl 4-(5,5-difluoro-7-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}-2,7-diazaspiro[3.5]nonan-2-yl)piperidine-1-carboxylate. To a stirred solution of 6-[4-({5,5-difluoro-2,7-diazaspiro[3.5]nonan-7-yl}methyl)-2,3-dihydroindol-1-yl]-N-[(1R,2R)-2-methoxycyclobutyl]-8-(methylamino)imidazo[1,2-b]pyridazine-3-carboxamide trifluoroacetate (150 mg, 0.226 mmol, Intermediate WU) in DMSO (4 mL) was added Et₃N (0.1 mL, 0.7 mmol) at rt under nitrogen atmosphere. The resulting mixture was stirred at rt for 5 min under nitrogen atmosphere. To the above mixture were added tert-butyl 4-oxopiperidine-1-carboxylate (44.97 mg, 0.226 mmol) and AcOH (0.06 mL, 1.13 mmol) at rt. The resulting mixture was stirred at 50° C. for an additional 30 min. The mixture was allowed to cool down to rt. To the above mixture was added sodium cyanoboranuide (42.54 mg, 0.678 mmol) at rt. The resulting mixture was stirred at 50° C. for an additional 2 h. On completion, the mixture was cooled to rt and concentrated under reduced pressure. The residue was purified by reverse flash chromatography (column, C18 silica gel; mobile phase A: water (10 mmol/L NH₄HCO₃), mobile phase B: ACN, 30% to 50% gradient in 30 min; detector, UV 254 nm, the fractions were collected at 45% and concentrated under reduced pressure) to afford the title compound (120 mg, 71% yield) as a white solid. LC/MS (ESI, m/z): [(M+H)]⁺=750.4.

Step 2—6-(4-{[5,5-Difluoro-2-(piperidin-4-yl)-2,7-diazaspiro[3.5]nonan-7-yl]methyl}-2,3-dihydroindol-1-yl)-N-[(1R,2R)-2-methoxycyclobutyl]-8-(methylamino)imidazo[1,2-b]pyridazine-3-carboxamide trifluoroacetate. To a stirred solution of tert-butyl 4-(5,5-difluoro-7-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}-2,7-diazaspiro[3.5]nonan-2-yl)piperidine-1-carboxylate (120 mg, 0.160 mmol) in DCM (2 mL) was added trifluoroacetic acid (1 mL) at rt under nitrogen atmosphere. The resulting mixture was stirred for 1 h at rt under nitrogen atmosphere. On completion, the mixture was concentrated under reduced pressure. The residue was purified by trituration with ethyl ether (10 mL) to afford the title compound (100 mg) as a white solid. LC/MS (ESI, m/z): [(M+H)]f=650.3.

### (S)-1-(2-chloro-5-(9-((3,3-difluoropiperidin-4-yl)methyl)-3,9-diazaspiro[5.5]undecane-3-carbonyl)-3-methylphenyl)dihydropyrimidine-2,4(1H,3H)-dione (Intermediate WW)

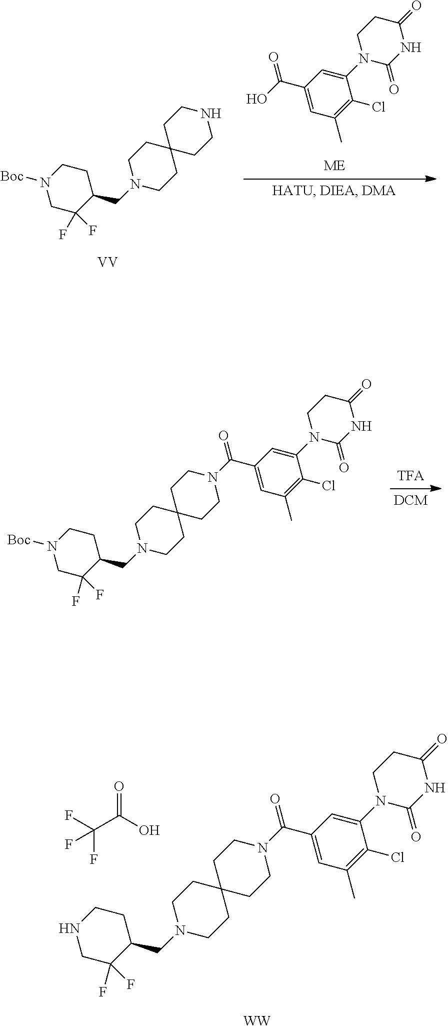

Step 1—Tert-butyl (S)-4-((9-(4-chloro-3-(2,4-dioxotetrahydropyrimidin-1(2H)-yl)-5-methylbenzoyl)-3,9-diazaspiro[5.5]undecan-3-yl)methyl)-3,3-difluoropiperidine-1-carboxylate. To a stirred solution of tert-butyl (4S)-4-{3,9-diazaspiro[5.5]undecan-3-ylmethyl}-3,3-difluoropiperidine-1-carboxylate (1.50 g, 3.87 mmol, Intermediate VV) and 4-chloro-3-(2,4-dioxo-1,3-diazinan-1-yl)-5-methylbenzoic acid (1.09 g, 3.87 mmol, Intermediate ME) in N,N-Dimethylacetamide (20 mL) were added HATU (2.20 g, 5.80 mmol) and DIEA (1.50 g, 11.61 mmol) in turns at rt under nitrogen atmosphere. The resulting mixture was stirred at rt for 1 h under nitrogen atmosphere. After completion of the reaction, the reaction mixture was purified directly by reverse phase flash chromatography (Column: WelFlash™ C8-I, 20-40 M, 120 g; Eluent A: Water (10 mmol/L NH₄HCO₃); Eluent B: ACN; Gradient: 40%-70% B in 40 min; Flow rate: 60 mL/min; Detector: 220/254 nm; desired fractions were collected at 60% B and concentrated under reduced pressure) to afford the title compound (2.00 g, 79% yield) as a yellow solid. LC/MS (ESI, m/z): [(M+1)]⁺=652.2.

Step 2—(S)-1-(2-chloro-5-(9-((3,3-difluoropiperidin-4-yl)methyl)-3,9-diazaspiro[5.5]undecane-3-carbonyl)-3-methylphenyl)dihydropyrimidine-2,4(1H,3H)-dione trifluoroacetate. To a stirred solution of tert-butyl (S)-4-((9-(4-chloro-3-(2,4-dioxotetrahydropyrimidin-1(2H)-yl)-5-methylbenzoyl)-3,9-diazaspiro[5.5]undecan-3-yl)methyl)-3,3-difluoropiperidine-1-carboxylate (2.00 g, 3.06 mmol) in DCM (15 mL) was added trifluoroacetic acid (5 mL) in portions at rt under nitrogen atmosphere. The resulting mixture was stirred at rt for 1 h under nitrogen atmosphere. On completion, the mixture was concentrated under reduced pressure. The residue was purified by trituration with Et₂O (50 mL) to give the title compound (1.50 g, 90% yield) as a yellow solid. LC/MS (ESI, m/z): [(M+1)]⁺=552.2.

### Tert-butyl (R)-4-((3,9-diazaspiro[5.5]undecan-3-yl)methyl)-3,3-difluoropiperidine-1-carboxylate (Intermediate WX)

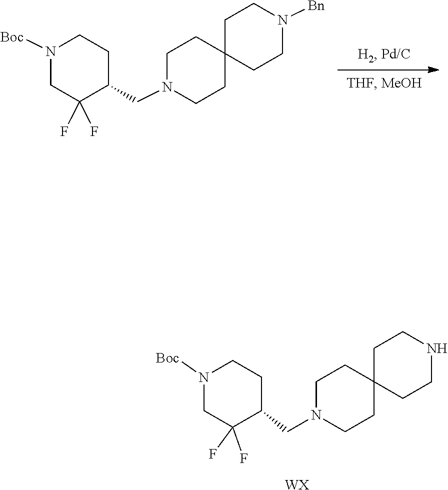

To a stirred mixture of tert-butyl (4R)-4-({9-benzyl-3,9-diazaspiro[5.5]undecan-3-yl}methyl)-3,3-difluoropiperidine-1-carboxylate (20 g, 40 mmol, synthesized via Steps 1-3 of Intermediate VV) in THF (80 mL) and methanol (80 mL) was added Pd/C (25 g, 230 mmol) in portions at rt under nitrogen atmosphere. The resulting mixture was stirred at rt for 4 h under hydrogen atmosphere. On completion, the mixture was filtered, and the filter cake was washed with MeOH (3×200 mL). The filtrate was concentrated under reduced pressure to afford the title compound (13.0 g, 80% yield) as a grey solid. LC/MS (ESI, m/z): [(M+1)]f=388.3.

### (R)-1-(2-chloro-5-(9-((3,3-difluoropiperidin-4-yl)methyl)-3,9-diazaspiro[5.5]undecane-3-carbonyl)-3-methylphenyl)dihydropyrimidine-2,4(1H,3H)-dione (Intermediate WY)

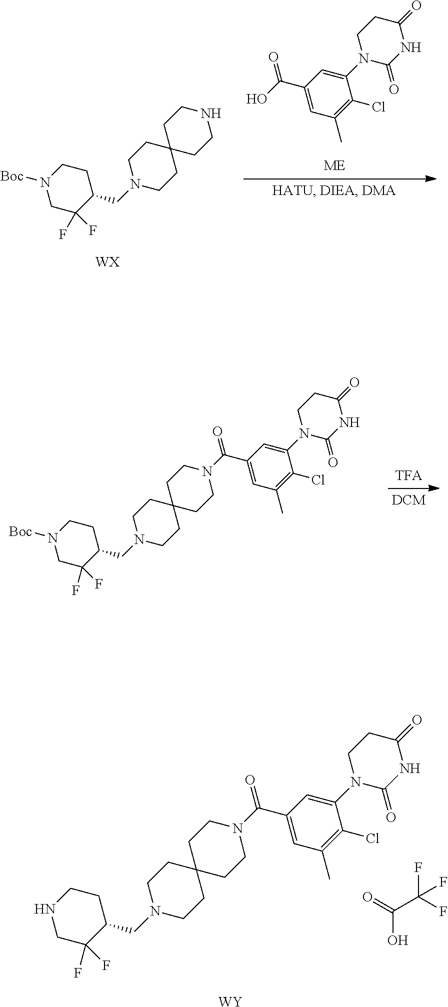

Step 1—Tert-butyl (R)-4-((9-(4-chloro-3-(2,4-dioxotetrahydropyrimidin-1(2H)-yl)-5-methylbenzoyl)-3,9-diazaspiro[5.5]undecan-3-yl)methyl)-3,3-difluoropiperidine-1-carboxylate. To a stirred solution of tert-butyl (R)-4-((3,9-diazaspiro[5.5]undecan-3-yl)methyl)-3,3-difluoropiperidine-1-carboxylate (1.00 g, 2.58 mmol, Intermediate WX) and 4-chloro-3-(2,4-dioxotetrahydropyrimidin-1(2H)-yl)-5-methylbenzoic acid (729 mg, 2.58 mmol, Intermediate ME) in N,N-Dimethylacetamide (10 mL) were added DIEA (1.00 g, 7.74 mmol) and HATU (1.47 g, 3.87 mmol) at 0° C. under nitrogen atmosphere. The resulting mixture was stirred for 1 h at 25° C. under nitrogen atmosphere. After completion of the reaction, the reaction mixture was purified directly by reverse phase flash chromatography (Column: WelFlash™ C18-J, 20-40 μm, 330 g; Eluent A: Water (plus 10 mmol/L NH₄HCO₃); Eluent B: ACN; Gradient: 35%-55% B in 25 min; Flow rate: 80 mL/min; Detector: 220/254 nm; desired fractions were collected at 47% B and concentrated under reduced pressure) to afford the title compound (1.20 g, 71% yield) as a white solid. LC/MS (ESI, m/z): [(M+H)]⁺=652.2.

Step 2—(R)-1-(2-chloro-5-(9-((3,3-difluoropiperidin-4-yl)methyl)-3,9-diazaspiro[5.5]undecane-3-carbonyl)-3-methylphenyl)dihydropyrimidine-2,4(1H,3H)-dione trifluoroacetate. To a stirred solution of tert-butyl (R)-4-((9-(4-chloro-3-(2,4-dioxotetrahydropyrimidin-1(2H)-yl)-5-methylbenzoyl)-3,9-diazaspiro[5.5]undecan-3-yl)methyl)-3,3-difluoropiperidine-1-carboxylate (1.20 g, 1.84 mmol) in DCM (12 mL) was added trifluoroacetic acid (4 mL) in portions at it under nitrogen atmosphere. The resulting mixture was stirred for 1 h at rt under nitrogen atmosphere. On completion, the mixture was concentrated under vacuum. The residue was purified by trituration with Et₂O (60 mL) to give the title compound (1.90 g) as a white solid. LC/MS (ESI, m/z): [(M+H)]⁺=552.2. Benzyl 4-(piperazin-1-yl)-2,3-dihydroindole-1-carboxylate (Intermediate WZ)

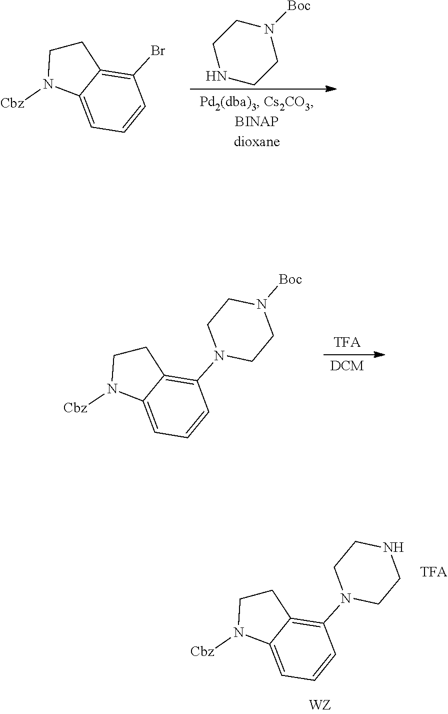 [SMILES: O=C(OCc1ccccc1)N1CCc2c(Br)cccc21 ; CC(C)(C)OC(=O)N1CCNCC1 ; CC(C)(C)OC(=O)N1CCN(c2cccc3c2CCN3C(=O)OCc2ccccc2)CC1 ; O=C(OCc1ccccc1)N1CCc2c(N3CCNCC3)cccc21]

Step 1—Benzyl 4-[4-(tert-butoxycarbonyl)piperazin-1-yl]-2,3-dihydroindole-1-carboxylate. To a stirred mixture of benzyl 4-bromo-2,3-dihydroindole-1-carboxylate (6 g, 18 mmol, synthesized via Step 1 of Intermediate OK) and tert-butyl piperazine-1-carboxylate (4.04 g, 21.7 mmol) in 1,4-dioxane (20 mL) were added Cs₂CO₃ (17.65 g, 54.19 mmol), Pd₂(dba)₃ (1.65 g, 1.81 mmol) and BINAP (2.25 g, 3.612 mmol) at it under nitrogen atmosphere. The resulting mixture was stirred at 100° C. for 1 h under nitrogen atmosphere. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 65%-95% B in 25 min; Flow rate: 80 mL/min; Detector: 220/254 nm; desired fractions were collected at 78% B and concentrated under reduced pressure) to afford the title compound (4.2 g, 53% yield) as a white solid. LC/MS (ESI, m/z): [(M+H)]⁺=438.2.

Step 2—Benzyl 4-(piperazin-1-yl)-2,3-dihydroindole-1-carboxylate trifluoroacetate. To a stirred solution of benzyl 4-[4-(tert-butoxycarbonyl)piperazin-1-yl]-2,3-dihydroindole-1-carboxylate (4.2 g, 9.6 mmol) in DCM (10 mL) was added TFA (5 mL) at rt under nitrogen atmosphere. The resulting mixture was stirred at rt for 1 h under nitrogen atmosphere. On completion, the mixture was concentrated under reduced pressure. The residue was purified by trituration with Et₂O (30 mL) to give the title compound (4.0 g) as a brown solid. LC/MS (ESI, m/z): [(M+H)]f=338.4. Benzyl 4-(4-{[(4R)-1-(tert-butoxycarbonyl)-3,3-difluoropiperidin-4-yl]methyl}piperazin-1-yl)-2,3-dihydroindole-1-carboxylate (Intermediate XA) and Benzyl 4-(4-{[(4S)-1-(tert-butoxycarbonyl)-3,3-difluoropiperidin-4-yl]methyl}piperazin-1-yl)-2,3-dihydroindole-1-carboxylate (Intermediate XB)

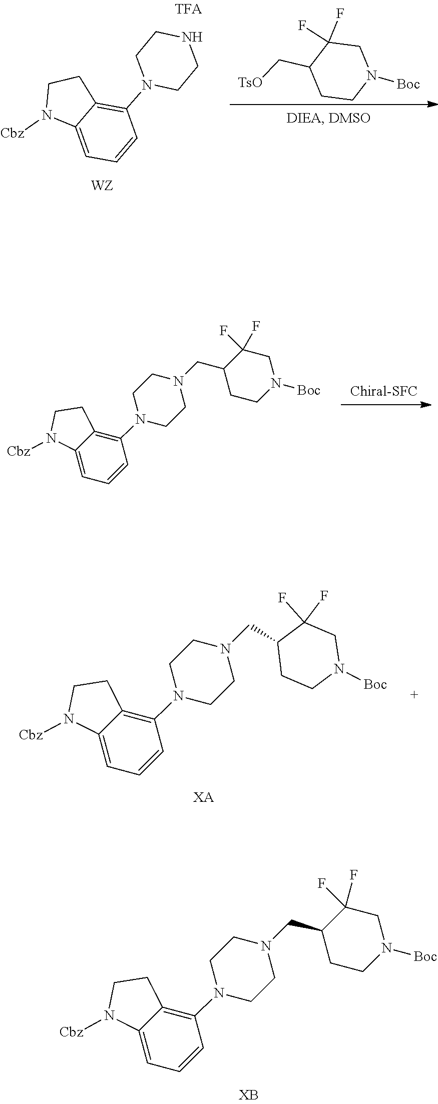

Step 1—Benzyl 4-(4-{[1-(tert-butoxycarbonyl)-3,3-difluoropiperidin-4-yl]methyl}piperazin-1-yl)-2,3-dihydroindole-1-carboxylate. To a stirred solution of benzyl 4-(piperazin-1-yl)-2,3-dihydroindole-1-carboxylate trifluoroacetate (4 g, 9 mmol, Intermediate WZ) in DMSO (40 mL) were added DIEA (4.80 mL, 27.6 mmol) and tert-butyl 3,3-difluoro-4-{[(4-methylbenzenesulfonyl)oxy]methyl}piperidine-1-carboxylate (3.72 g, 9.19 mmol, synthesized via Step 1 of Intermediate VV) at rt under nitrogen atmosphere. The resulting mixture was stirred at 100° C. for 16 h under nitrogen atmosphere. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 55%-100% B in 25 min; Flow rate: 80 mL/min; Detector: 220/254 nm; desired fractions were collected at 100% B and concentrated under reduced pressure) to afford the title compound (3.0 g, 57% yield) as a brown solid. LC/MS (ESI, m/z): [(M+H)]⁺=571.3.

Step 2—Benzyl 4-(4-{[(4R)-1-(tert-butoxycarbonyl)-3,3-difluoropiperidin-4-yl]methyl}piperazin-1-yl)-2,3-dihydroindole-1-carboxylate. Benzyl 4-(4-{[1-(tert-butoxycarbonyl)-3,3-difluoropiperidin-4-yl]methyl}piperazin-1-yl)-2,3-dihydroindole-1-carboxylate (3.0 g) was separated by Prep-Chiral-HPLC with the following conditions: Column: CHIRALART Cellulose-SC, 5*25 cm, 10 m; Gradient: isocratic; Wave Length: 220 nm; RT1(min): 13; RT2(min): 15; Sample Solvent: EtOH; Injection Volume: 10 mL) to afford the first eluting peak benzyl 4-(4-{[(4R)-1-(tert-butoxycarbonyl)-3,3-difluoropiperidin-4-yl]methyl}piperazin-1-yl)-2,3-dihydroindole-1-carboxylate (1.1 g, 37% yield) as a brown solid and the second eluting peak benzyl 4-(4-{[(4S)-1-(tert-butoxycarbonyl)-3,3-difluoropiperidin-4-yl]methyl}piperazin-1-yl)-2,3-dihydroindole-1-carboxylate (900 mg, 30% yield) as a brown solid. LC/MS (ESI, m/z): [(M+H)]⁺=571.3 for both isomers. The absolute stereochemistry of the enantiomers was assigned arbitrarily.

Benzyl 4-(4-{[(4R)-3,3-difluoropiperidin-4-yl]methyl}piperazin-1-yl)-2,3-dihydroindole-1-carboxylate (Intermediate XC)

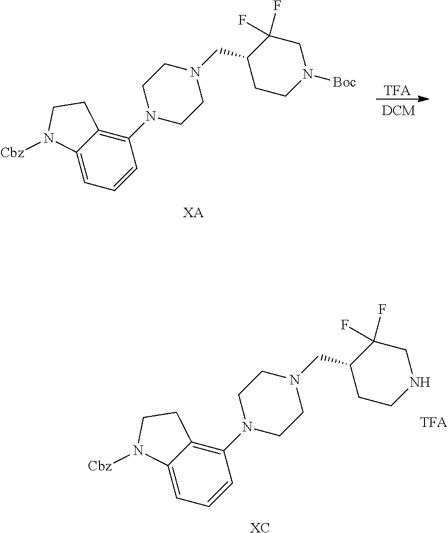

To a stirred solution of benzyl 4-(4-{[(4R)-1-(tert-butoxycarbonyl)-3,3-difluoropiperidin-4-yl]methyl}piperazin-1-yl)-2,3-dihydroindole-1-carboxylate (1.1 g, 1.9 mmol, Intermediate XA) in DCM (5 mL) was added TFA (2.5 mL) at rt under nitrogen atmosphere. The resulting mixture was stirred at rt for 1 h under nitrogen atmosphere. On completion, the mixture was concentrated under reduced pressure. The residue was purified by trituration with Et₂O (30 mL) to give the title compound (1.0 g) as a brown solid. LC/MS (ESI, m/z): [(M+H)]⁺=471.2.

### Tert-butyl (4R)-4-[(4-{1-[(benzyloxy)carbonyl]-2,3-dihydroindol-4-yl}piperazin-1-yl)methyl]-3,3-difluoro-[1,4′-bipiperidine]-1′-carboxylate (Intermediate XD)

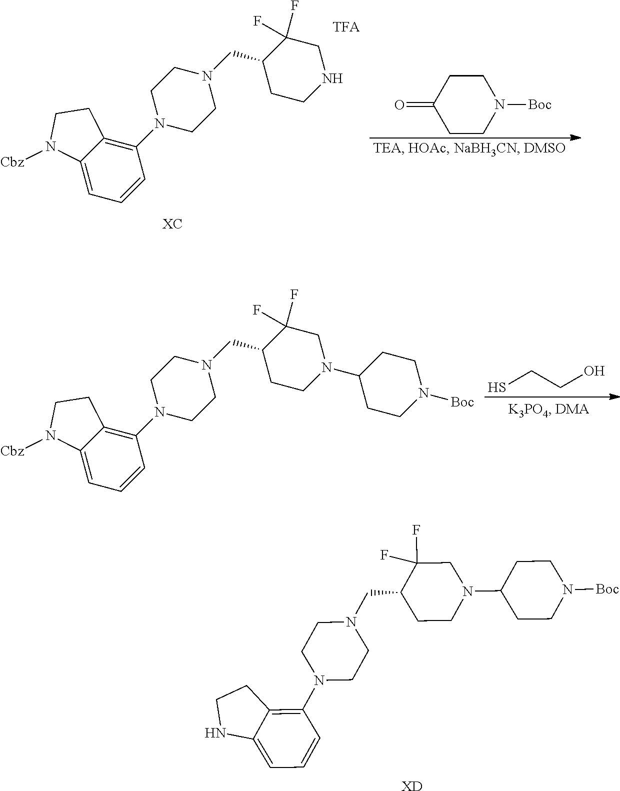

Step 1—Tert-butyl (4R)-4-[(4-{1-[(benzyloxy)carbonyl]-2,3-dihydroindol-4-yl}piperazin-1-yl)methyl]-3,3-difluoro-[1,4′-bipiperidine]-1′-carboxylate. To a stirred solution of benzyl 4-(4-{[(4R)-3,3-difluoropiperidin-4-yl]methyl}piperazin-1-yl)-2,3-dihydroindole-1-carboxylate trifluoroacetate (1.0 g, 1.8 mmol, Intermediate XC) in DMSO (10 mL) was added Et₃N (0.73 mL, 5.28 mmol) at rt under nitrogen atmosphere. To the above mixture were added tert-butyl 4-oxopiperidine-1-carboxylate (700.86 mg, 3.518 mmol) and AcOH (0.50 mL, 8.80 mmol) at rt. The resulting mixture was stirred at 50° C. for 1 h. To the above mixture was added sodium cyanoboranuide (552.60 mg, 8.795 mmol) at rt. The resulting mixture was stirred at 50° C. for an additional 16 h. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 80%-100% B in 15 min; Flow rate: 80 mL/min; Detector: 220/254 nm; desired fractions were collected at 100% B and concentrated under reduced pressure) to afford the title compound (510 mg, 44% yield) as an off-white solid. LC/MS (ESI, m/z): [(M+H)]⁺=654.3.

Step 2—Tert-butyl (4R)-4-[(4-{1-[(benzyloxy)carbonyl]-2,3-dihydroindol-4-yl}piperazin-1-yl)methyl]-3,3-difluoro-[1,4′-bipiperidine]-1′-carboxylate. To a stirred solution of tert-butyl (4R)-4-[(4-{1-[(benzyloxy)carbonyl]-2,3-dihydroindol-4-yl}piperazin-1-yl)methyl]-3,3-difluoro-[1,4′-bipiperidine]-1′-carboxylate (510 mg, 0.780 mmol) in N,N-Dimethylacetamide (10 mL) were added K₃PO₄ (662.30 mg, 3.120 mmol) and β-mercaptoethanol (243.78 mg, 3.120 mmol) at rt under nitrogen atmosphere. The resulting mixture was stirred at 80° C. for 16 h under nitrogen atmosphere. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 60%-90% B in 20 min; Flow rate: 80 mL/min; Detector: 220/254 nm; desired fractions were collected at 84% B and concentrated under reduced pressure) to afford the title compound (320 mg, 79% yield) as a white solid. LC/MS (ESI, m/z): [(M+H)]⁺=520.3.

### 6-[4-(4-{[(4R)-3,3-difluoro-[1,4′-bipiperidin]-4-yl]methyl}piperazin-1-yl)-2,3-dihydroindol-1-yl]-N-[(1R,2R)-2-methoxycyclobutyl]-8-(methylamino)imidazo[1,2-b]pyridazine-3-carboxamide (Intermediate XE)

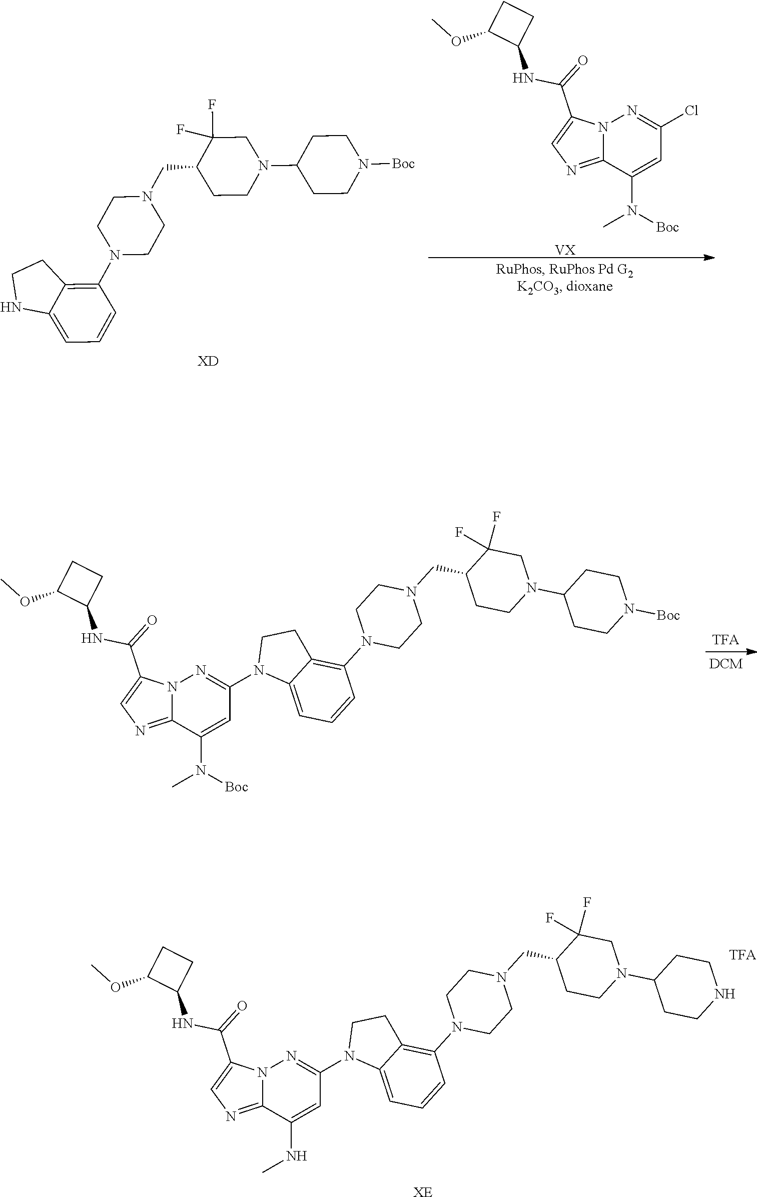

Step 1—Tert-butyl (4R)-4-{[4-(1-{8-[(tert-butoxycarbonyl)(methyl)amino]-3-{[(1R,2R)-2-methoxycyclobutyl]carbamoyl}imidazo[1,2-b]pyridazin-6-yl}-2,3-dihydroindol-4-yl)piperazin-1l-yl]methyl}-3,3-difluoro-[1,4′-bipiperidine]-1′-carboxylate. To a stirred mixture of tert-butyl (4R)-4-{[4-(2,3-dihydro-1H-indol-4-yl)piperazin-1-yl]methyl}-3,3-difluoro-[1,4′-bipiperidine]-1′-carboxylate (320 mg, 0.616 mmol, Intermediate XD) and tert-butyl N-(6-chloro-3-{[(1R,2R)-2-methoxycyclobutyl]carbamoyl}imidazo[1,2-b]pyridazin-8-yl)-N-methylcarbamate (252.38 mg, 0.616 mmol, Intermediate VX) in 1,4-dioxane (10 mL) were added K2CO₃ (255.30 mg, 1.848 mmol), RuPhos (57.47 mg, 0.123 mmol) and RuPhos Pd G2 (95.66 mg, 0.123 mmol) at rt under nitrogen atmosphere. The resulting mixture was stirred at 100° C. for 2 h under nitrogen atmosphere. On completion, the mixture was concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN: Gradient: 75%-100% B in 20 min: Flow rate: 80 mL/min: Detector: 220/254 nm: desired fractions were collected at 100% B and concentrated under reduced pressure) to afford the title compound (540 mg, 98% yield) as a brown solid. LC/MS (ESI, m/z): [(M+H)]=893.4.

Step 2—6-[4-(4-{[(4R)-3,3-difluoro-[1,4′-bipiperidin]-4-yl]methyl}piperazin-1-yl)-2,3-dihydroindol-1-yl]-N-[(1R,2R)-2-methoxycyclobutyl]-8-(methylamino)imidazo[1,2-b]pyridazine-3-carboxamide trifluoroacetate. To a stirred solution of tert-butyl (4R)-4-{[4-(1-{8-[(tert-butoxycarbonyl)(methyl)amino]-3-{[(1R,2R)-2-methoxycyclobutyl]carbamoyl}imidazo[1,2-b]pyridazin-6-yl}-2,3-dihydroindol-4-yl)piperazin-1-yl]methyl}-3,3-difluoro-[1,4′-bipiperidine]-1′-carboxylate (540 mg, 0.605 mmol) in DCM (6 mL) was added TFA (3 mL) at rt under nitrogen atmosphere. On completion, the mixture was cooled to rt and concentrated under reduced pressure. The residue was purified by trituration with Et₂O (20 mL) to give the title compound (550 mg) as a white solid. LC/MS (ESI, m/z): [(M+H)]⁺=693.4. Benzyl 4-(4-{[(4S)-3,3-difluoropiperidin-4-yl]methyl}piperazin-1-yl)-2,3-dihydroindole-1-carboxylate (Intermediate XF)

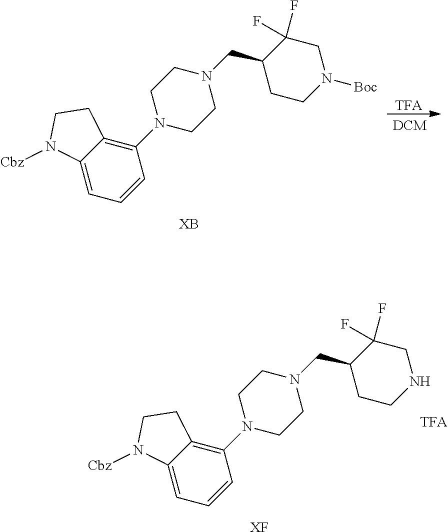

To a stirred mixture of benzyl 4-(4-{[(4S)-1-(tert-butoxycarbonyl)-3,3-difluoropiperidin-4-yl]methyl}piperazin-1-yl)-2,3-dihydroindole-1-carboxylate (900 mg, 1.577 mmol, Intermediate XB) in DCM (10 mL) was added trifluoroacetic acid (5 mL) at rt under nitrogen atmosphere. The resulting mixture was stirred at rt for 1 h. On completion, the mixture was concentrated under reduced pressure. The residue was purified by trituration with Et₂O (30 mL) to give the title compound (1.05 g) as a white solid. LC/MS (ESI, m/z): [(M+H)]=471.2.

### Tert-butyl (4S)-4-{[4-(2,3-dihydro-1H-indol-4-yl)piperazin-1-yl]methyl}-3,3-difluoro-[1,4′-bipiperidine]-1′-carboxylate (Intermediate XG)

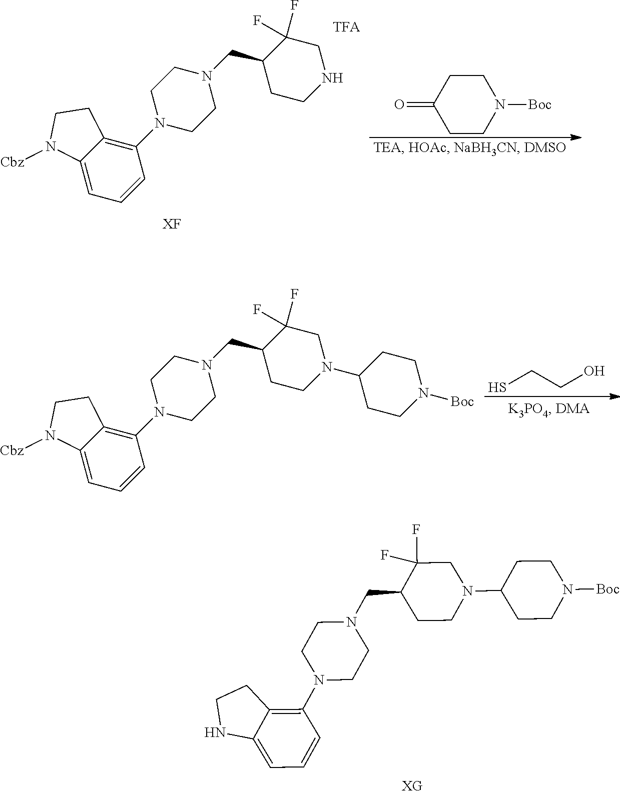

Step 1—Tert-butyl (4S)-4-[(4-{1-[(benzyloxy)carbonyl]-2,3-dihydroindol-4-yl}piperazin-1-yl)methyl]-3,3-difluoro-[1,4′-bipiperidine]-1′-carboxylate. To a stirred solution of benzyl 4-(4-{[(4S)-3,3-difluoropiperidin-4-yl]methyl}piperazin-1-yl)-2,3-dihydroindole-1-carboxylate trifluoroacetate (1.05 g, 1.80 mmol, Intermediate XF) in DMSO (10 mL) was added Et₃N (0.75 mL, 5.388 mmol) at rt under nitrogen atmosphere. The resulting mixture was stirred at rt for 5 min under nitrogen atmosphere. To the above mixture were added tert-butyl 4-oxopiperidine-1-carboxylate (0.36 g, 1.80 mmol) and AcOH (0.74 mL, 12.3 mmol) at rt. The resulting mixture was stirred at 50° C. for an additional 30 min. To the above mixture was added sodium cyanoboranuide (0.23 g, 3.60 mmol) at rt. The resulting mixture was stirred at 50° C. for an additional 2 h. On completion, the mixture was cooled to rt and concentrated under reduced pressure. The residue was purified by reverse flash chromatography (column, C18 silica gel; mobile phase A: water (10 mmol/L NH₄HCO₃), mobile phase B: ACN, 30% to 50% gradient in 30 min; detector, UV 254 nm; the fractions were collected at 45% and concentrated under reduced pressure) to afford the title compound (700 mg, 60% yield) as a white solid. LC/MS (ESI, m/z): [(M+H)]⁺=654.3.

Step 2—Tert-butyl (4S)-4-{[4-(2,3-dihydro-1H-indol-4-yl)piperazin-1-yl]methyl}-3,3-difluoro-[1,4′-bipiperidine]-1′-carboxylate. To a stirred mixture of tert-butyl (4S)-4-[(4-{l-[(benzyloxy)carbonyl]-2,3-dihydroindol-4-yl}piperazin-1-yl)methyl]-3,3-difluoro-[1,4′-bipiperidine]-1′-carboxylate (700 mg, 1.07 mmol) and K₃PO₄ (909.04 mg, 4.284 mmol) in N,N-Dimethylacetamide (10 mL) was added 0-mercaptoethanol (167.30 mg, 2.142 mmol) at rt under nitrogen atmosphere. The resulting mixture was stirred at 70° C. for 16 h under nitrogen atmosphere. On completion, the mixture was concentrated under reduced pressure. The residue was purified by reverse flash chromatography (column, C18 silica gel; mobile phase A: water (10 mmol/L NH₄HCO₃), mobile phase B: ACN, 30% to 50% gradient in 30 min; detector, UV 254 nm; the fractions were collected at 45% and concentrated under reduced pressure) to afford the title compound (400 mg, 72%) as a white solid. LC/MS (ESI, m/z): [(M+H)]⁺=520.2.

### 6-[4-(4-{[(4S)-3,3-difluoro-[1,4′-bipiperidin]-4-yl]methyl}piperazin-1-yl)-2,3-dihydroindol-1-yl]-N-[(1R,2R)-2-methoxycyclobutyl]-8-(methylamino)imidazo[1,2-b]pyridazine-3-carboxamide (Intermediate XH)

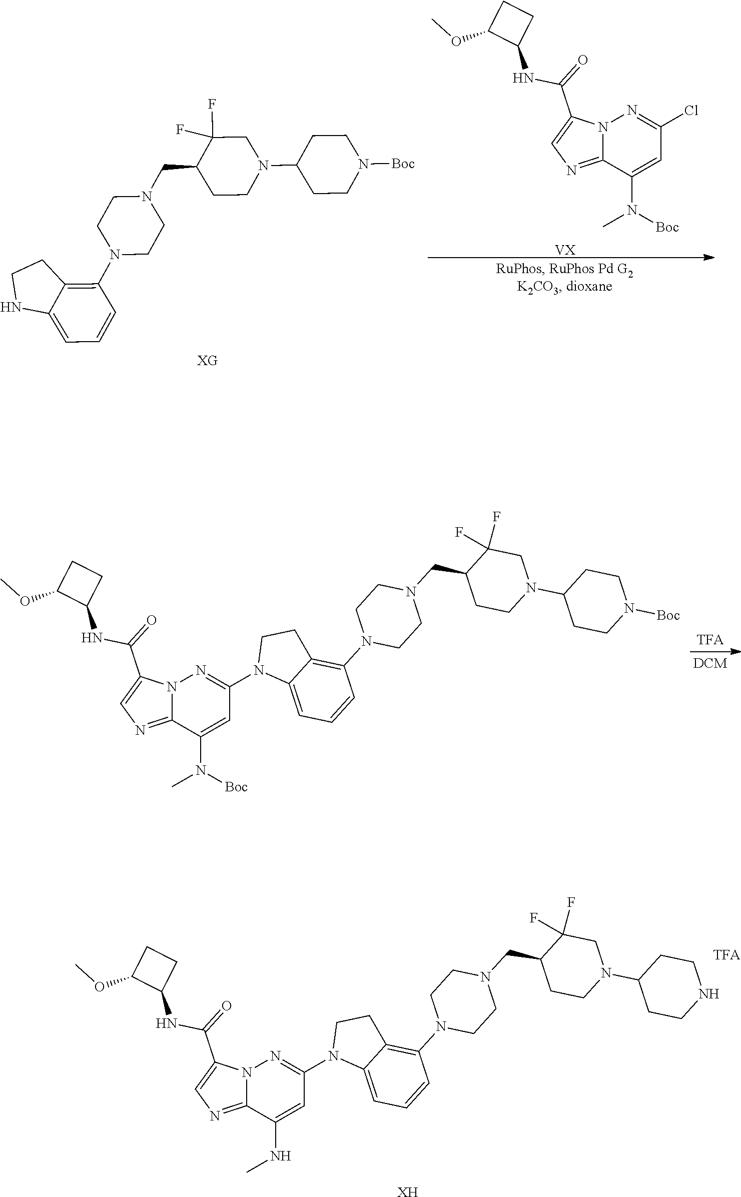

Step 1—Tert-butyl (4S)-4-{[4-(1-{8-[(tert-butoxycarbonyl)(methyl)amino]-3-{[(1R,2R)-2-methoxycyclobutyl]carbamoyl}imidazo[1,2-b]pyridazin-6-yl}-2,3-dihydroindol-4-yl)piperazin-1-yl]methyl}-3,3-difluoro-[1,4′-bipiperidine]-1′-carboxylate. To a stirred mixture of tert-butyl (4S)-4-{[4-(2,3-dihydro-1H-indol-4-yl)piperazin-1-yl]methyl}-3,3-difluoro-[1,4′-bipiperidine]-1′-carboxylate (400 mg, 0.770 mmol, Intermediate XG) and tert-butyl N-(6-chloro-3-{[(1R,2R)-2-methoxycyclobutyl]carbamoyl}imidazo[1,2-b]pyridazin-8-yl)-N-methylcarbamate (315.08 mg, 0.770 mmol, Intermediate VX) in 1,4-dioxane (8 mL) were added K₂CO₃ (319.13 mg, 2.310 mmol), RuPhos (71.84 mg, 0.154 mmol) and RuPhos Pd G₂ (119.57 mg, 0.154 mmol) at rt under nitrogen atmosphere. The resulting mixture was stirred at 100° C. for 2 h under nitrogen atmosphere. On completion, the mixture was cooled to rt and concentrated under reduced pressure. The residue was purified by reverse flash chromatography (column, C18 silica gel; mobile phase A: water (10 mmol/L NH₄HCO₃), mobile phase B: ACN, 30% to 50% gradient in 30 min; detector, UV 254 nm; the fractions were collected at 45% and concentrated under reduced pressure) to afford the title compound (600 mg, 87.30%) as a yellow solid. LC/MS (ESI, m/z): [(M+H)]⁺=893.4.

Step 2—6-[4-(4-{[(4S)-3,3-difluoro-[1,4′-bipiperidin]-4-yl]methyl}piperazin-1-yl)-2,3-dihydroindol-1-yl]-N-[(1R,2R)-2-methoxycyclobutyl]-8-(methylamino)imidazo[1,2-b]pyridazine-3-carboxamide trifluoroacetate. To a stirred solution of tert-butyl (4S)-4-{[4-(1-{8-[(tert-butoxycarbonyl)(methyl)amino]-3-{[(1R,2R)-2-methoxycyclobutyl]carbamoyl}imidazo[1,2-b]pyridazin-6-yl}-2,3-dihydroindol-4-yl)piperazin-1-yl]methyl}-3,3-difluoro-[1,4′-bipiperidine]-1′-carboxylate (650 mg, 0.712 mmol) in DCM (6 mL) was added trifluoroacetic acid (3 mL) at rt under nitrogen atmosphere. The resulting mixture was stirred at rt for 1 h. On completion, the mixture was concentrated under reduced pressure. The residue was purified by trituration with Et₂O (30 mL) to give the title compound (800 mg) as a grey solid. LC/MS (ESI, m/z): [(M+H)]-=693.4.

### N-cyclobutyl-6-(4-((1,1-difluoro-9-(piperidin-4-yl)-3,9-diazaspiro[5.5]undecan-3-yl)methyl)indolin-1-yl)-8-(methylamino)imidazo[1,2-b]pyridazine-3-carboxamide (Intermediate XI)

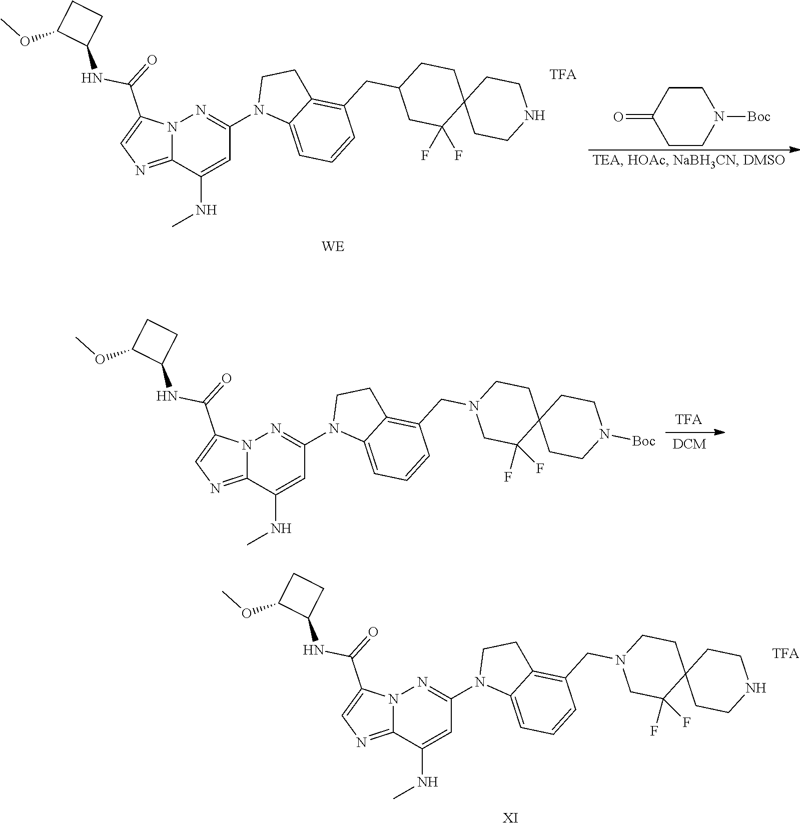

Step 1—Tert-butyl 4-(7,7-difluoro-9-((1-(3-(((1R,2R)-2-methoxycyclobutyl)carbamoyl)-8-(methylamino)imidazo[1,2-b]pyridazin-6-yl)indolin-4-yl)methyl)-3,9-diazaspiro[5.5]undecan-3-yl)piperidine-1-carboxylate. To a stirred mixture of 6-(4-((1,1-difluoro-3,9-diazaspiro[5.5]undecan-3-yl)methyl)indolin-1-yl)-N-((1R,2R)-2-methoxycyclobutyl)-8-(methylamino)imidazo[1,2-b]pyridazine-3-carboxamide trifluoroacetate (150 mg, 0.217 mmol, Intermediate WE) and Et₃N (0.09 mL, 0.651 mmol) in DMSO (3 mL) were added AcOH (0.1 mL, 1.1 mmol) and tert-butyl 4-oxopiperidine-1-carboxylate (43 mg, 0.22 mmol) at rt under nitrogen atmosphere. The resulting mixture was stirred at 50° C. for 0.5 h under nitrogen atmosphere. The mixture was allowed to cool down to rt. To the above mixture was added sodium cyanoboranuide (41 mg, 0.651 mmol) at rt. The resulting mixture was stirred at 50° C. for an additional 1.5 h. On completion, the mixture was cooled to rt and concentrated under reduced pressure. The residue was purified by reverse phase flash chromatography (Column: WelFlash™ C18-I, 20-40 μm, 120 g; Eluent A: Water (plus 10 mmol/L FA); Eluent B: ACN; Gradient: 35%-55% B in 15 min; Flow rate: 50 mL/min; Detector: 220/254 nm; desired fractions were collected at 50% B and concentrated under reduced pressure) to afford the title compound (90 mg, 53% yield) as a white solid. LC/MS (ESI, m/z): [(M+H)]⁺=778.4.

Step 2—N-cyclobutyl-6-(4-((1,1-difluoro-9-(piperidin-4-yl)-3,9-diazaspiro[5.5]undecan-3-yl)methyl)indolin-1-yl)-8-(methylamino)imidazo[1,2-b]pyridazine-3-carboxamide trifluoroacetate. To a stirred solution of tert-butyl 4-(7,7-difluoro-9-((1-(3-(((1R,2R)-2-methoxycyclobutyl)carbamoyl)-8-(methylamino)imidazo[1,2-b]pyridazin-6-yl)indolin-4-yl)methyl)-3,9-diazaspiro[5.5]undecan-3-yl)piperidine-1-carboxylate (90 mg, 0.12 mmol) in DCM (2 mL) was added trifluoroacetic acid (1 mL) at rt under nitrogen atmosphere. The resulting mixture was stirred for 1 h at rt under nitrogen atmosphere. On completion, the mixture was concentrated under reduced pressure. The residue was purified by trituration with Et₂O (10 mL) to afford the title compound (127 mg) as a white solid. LC/MS (ESI, m/z): [(M+H)]=678.3.

### 4-(5-(1,3-Dioxolan-2-yl)-3-fluoropyridin-2-yl)indoline (Intermediate XJ)

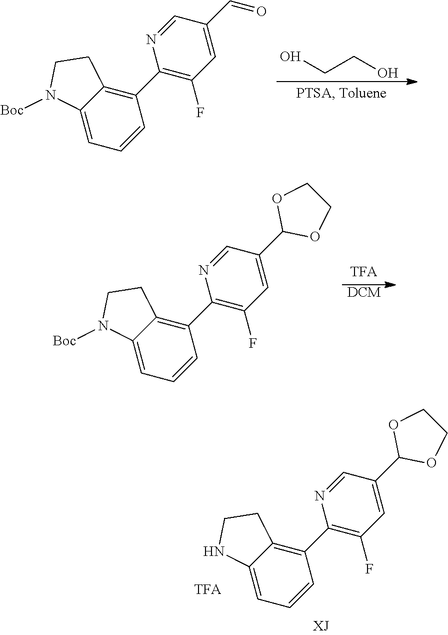

Step 1—tert-butyl 4-(5-(1,3-dioxolan-2-yl)-3-fluoropyridin-2-yl)indoline-1-carboxylate. To a stirred solution of tert-butyl 4-(3-fluoro-5-formylpyridin-2-yl)-2,3-dihydroindole-1-carboxylate (12.00 g, 35.05 mmol, synthesized via Step 1 of Intermediate MB) and ethylene glycol (4.35 g, 70.1 mmol) in toluene (150 mL) was added PTSA (0.60 g, 3.50 mmol) in portions at it under nitrogen atmosphere. The resulting mixture was stirred for 16 h at 110° C. under nitrogen atmosphere. On completion, the mixture was cooled to rt and concentrated under reduced pressure. The residue was purified by silica gel column chromatography, eluted with PE/EA (5:1), to afford the title compound (4.00 g, 29% yield) as a yellow oil. LC/MS (ESI, m/z): [(M+1)]⁺=387.4.

Step 2—4-(5-(1,3-Dioxolan-2-yl)-3-fluoropyridin-2-yl)indoline trifluoroacetate. To a stirred solution of tert-butyl 4-(5-(1,3-dioxolan-2-yl)-3-fluoropyridin-2-yl)indoline-1-carboxylate (1.00 g, 2.58 mmol) in DCM (10 mL) was added TFA (3 mL) in portions at rt under nitrogen atmosphere. The resulting mixture was stirred for 1 h at rt under nitrogen atmosphere. On completion, the mixture was concentrated under vacuum. The residue was purified by trituration with Et₂O (40 mL) to give the title compound (740 mg, 99% yield) as a yellow oil. LC/MS (ESI, m/z): [(M+1)]⁺=287.0.

### 6-(4-(5-(1,3-Dioxolan-2-yl)-3-fluoropyridin-2-yl)indolin-1-yl)-8-((4-methoxybenzyl)(methyl)amino)imidazo[1,2-b]pyridazine-3-carboxylic acid (Intermediate XK)

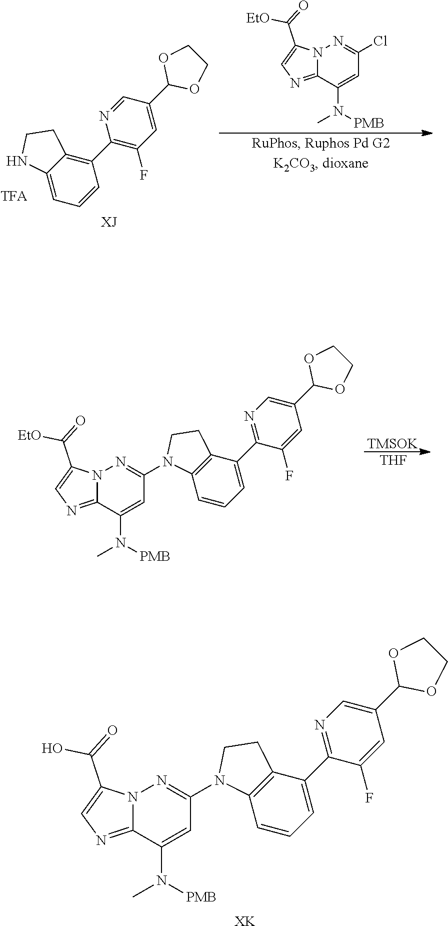

Step 1—Ethyl 6-(4-(5-(1,3-dioxolan-2-yl)-3-fluoropyridin-2-yl)indolin-1-yl)-8-((4-methoxybenzyl)(methyl)amino)imidazo[1,2-b]pyridazine-3-carboxylate. To a stirred solution of 4-(5-(1,3-dioxolan-2-yl)-3-fluoropyridin-2-yl)indoline trifluoroacetate (1.00 g, 3.49 mmol, Intermediate XJ) and ethyl 6-chloro-8-{[(4-methoxyphenyl)methyl](methyl)amino}imidazo[1,2-b]pyridazine-3-carboxylate (1.31 g, 3.49 mmol, synthesized via Steps 1-2 of Intermediate F) in dioxane (15 mL) were added RuPhos (0.33 g, 0.69 mmol), RuPhos-PdCl-2nd G (0.54 g, 0.69 mmol) and K₂CO₃ (1.45 g, 10.47 mmol) in turns at rt under nitrogen atmosphere. The resulting mixture was stirred for 1 h at 90° C. under nitrogen atmosphere. On completion, the mixture was cooled to it and concentrated under reduced pressure. The residue was purified by silica gel column chromatography, eluted with PE/EA (1:1), to afford the title compound (1.37 g, 62% yield) as a yellow solid. LC/MS (ESI, m/z): [(M+1)]⁺=625.4.

Step 2—6-(4-(5-(1,3-Dioxolan-2-yl)-3-fluoropyridin-2-yl)indolin-1-yl)-8-((4-methoxybenzyl)(methyl)amino)imidazo[1,2-b]pyridazine-3-carboxylic acid. To a stirred solution of ethyl 6-(4-(5-(1,3-dioxolan-2-yl)-3-fluoropyridin-2-yl)indolin-1-yl)-8-((4-methoxybenzyl)(methyl)amino)imidazo[1,2-b]pyridazine-3-carboxylate (1.37 g, 2.19 mmol) in THF (20 mL) was added potassium trimethylsilanolate (1.97 g, 15.35 mmol) in portions at rt under nitrogen atmosphere. The resulting mixture was stirred for 1 h at 50° C. under nitrogen atmosphere. On completion, the mixture was cooled to rt and acidified to pH 3 with 2 M HCl (aq.). The precipitated solids were collected by filtration and washed with water (2×200 mL) to give the title compound (400 mg, 30% yield) as a yellow solid. LC/MS (ESI, m/z): [(M+1)]=597.3.

### (R)—N-(2,2-difluorocyclopropyl)-6-(4-(3-fluoro-5-formylpyridin-2-yl)indolin-1-yl)-8-(methylamino)imidazo[1,2-b]pyridazine-3-carboxamide (Intermediate XL)

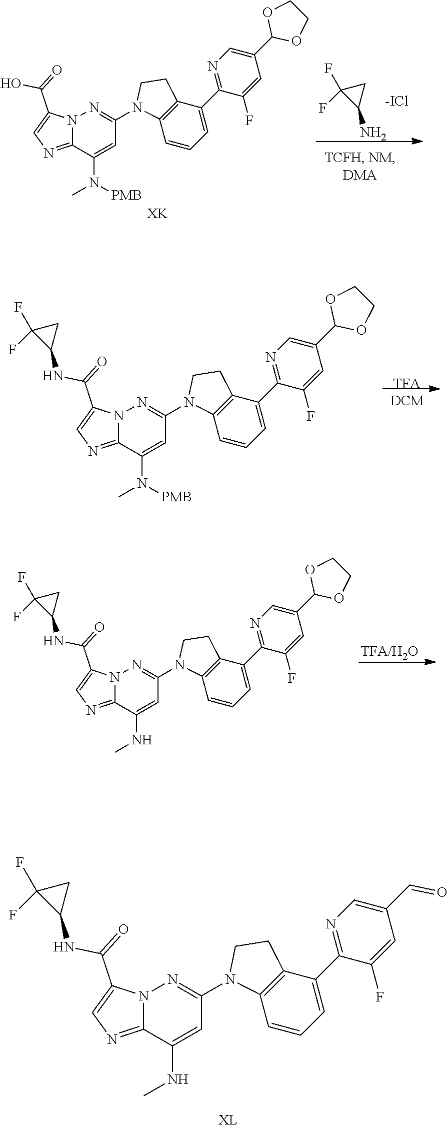

Step 1—(R)-6-(4-(5-(1,3-dioxolan-2-yl)-3-fluoropyridin-2-yl)indolin-1-yl)-N-(2,2-difluorocyclopropyl)-8-((4-methoxybenzyl)(methyl)amino)imidazo[1,2-b]pyridazine-3-carboxamide. To a stirred solution of 6-(4-(5-(1,3-dioxolan-2-yl)-3-fluoropyridin-2-yl)indolin-1-yl)-8-((4-methoxybenzyl)(methyl)amino)imidazo[1,2-b]pyridazine-3-carboxylic acid (400 mg, 0.670 mmol, Intermediate XK) and (1R)-2,2-difluorocyclopropan-1-amine hydrochloride (86 mg, 0.670 mmol, CAS #2089150-96-7) in DMA (4 mL) were added TCFH (282 mg, 1,005 mmol) and 1-methyl-1H-imidazole (137 mg, 1.68 mmol) in turns at rt under nitrogen atmosphere. The resulting mixture was stirred for 2 h at 50° C. under nitrogen atmosphere. After completion of the reaction, the reaction mixture was cooled to rt and purified directly by reverse phase flash chromatography (Column: WelFlash™ C18-I, 20-40 μm, 120 g; Eluent A: Water (10 mmol/L NH₄HCO₃); Eluent B: ACN; Gradient: 25%-55% B in 40 min; Flow rate: 60 mL/min; Detector: 220/254 nm; desired fractions were collected at 50% B and concentrated under reduced pressure) to afford the title compound (150 mg, 33% yield) as a yellow solid. LC/MS (ESI, m/z): [(M+1)]⁺=672.1.

Step 2—(R)-6-(4-(5-(1,3-dioxolan-2-yl)-3-fluoropyridin-2-yl)indolin-1-yl)-N-(2,2-difluorocyclopropyl)-8-(methylamino)imidazo[1,2-b]pyridazine-3-carboxamide trifluoroacetate. To a stirred solution of (R)-6-(4-(5-(1,3-dioxolan-2-yl)-3-fluoropyridin-2-yl)indolin-1-yl)-N-(2,2-difluorocyclopropyl)-8-((4-methoxybenzyl)(methyl)amino)imidazo[1,2-b]pyridazine-3-carboxamide (150 mg, 0.223 mmol) in DCM (3 mL) was added TFA (1 mL) dropwise at rt under nitrogen atmosphere. The resulting mixture was stirred for 1 h at rt under nitrogen atmosphere. Upon completion, the mixture was concentrated under vacuum. The residue was purified by trituration with Et₂O (20 mL) to give the title compound (100 mg, 81% yield) as a yellow solid. LC/MS (ESI, m/z): [(M+1)]=552.3.

Step 3—(R)—N-(2,2-difluorocyclopropyl)-6-(4-(3-fluoro-5-formylpyridin-2-yl)indolin-1-yl)-8-(methylamino)imidazo[1,2-b]pyridazine-3-carboxamide. To a stirred solution of (R)-6-(4-(5-(1,3-dioxolan-2-yl)-3-fluoropyridin-2-yl)indolin-1-yl)-N-(2,2-difluorocyclopropyl)-8-(methylamino)imidazo[1,2-b]pyridazine-3-carboxamide trifluoroacetate (150 mg, 0.272 mmol) in H₂O (2 mL) was added TFA (2 mL) in portions at it under nitrogen atmosphere. The resulting mixture was stirred for 2 h at 50° C. under nitrogen atmosphere. Upon completion, the mixture was concentrated under vacuum. The residue was purified by reverse phase flash chromatography (Column: WelFlash™ C18-I, 20-40 μm, 120 g; Eluent A: Water (10 mmol/L NH₄HCO₃); Eluent B: ACN; Gradient: 25%-55% B in 40 min; Flow rate: 60 mL/min; Detector: 220/254 nm; desired fractions were collected at 50% B and concentrated under reduced pressure) to afford the title compound (70 mg, 50% yield) as a yellow solid. LC/MS (ESI, m/z): [(M+1)]⁺=508.3.

### (R)-6-(4-(5-((5,5-difluoro-2,7-diazaspiro[3.5]nonan-2-yl)methyl)-3-fluoropyridin-2-yl)indolin-1-yl)-N-(2,2-difluorocyclopropyl)-8-(methylamino)imidazo[1,2-b]pyridazine-3-carboxamide (Intermediate XM)

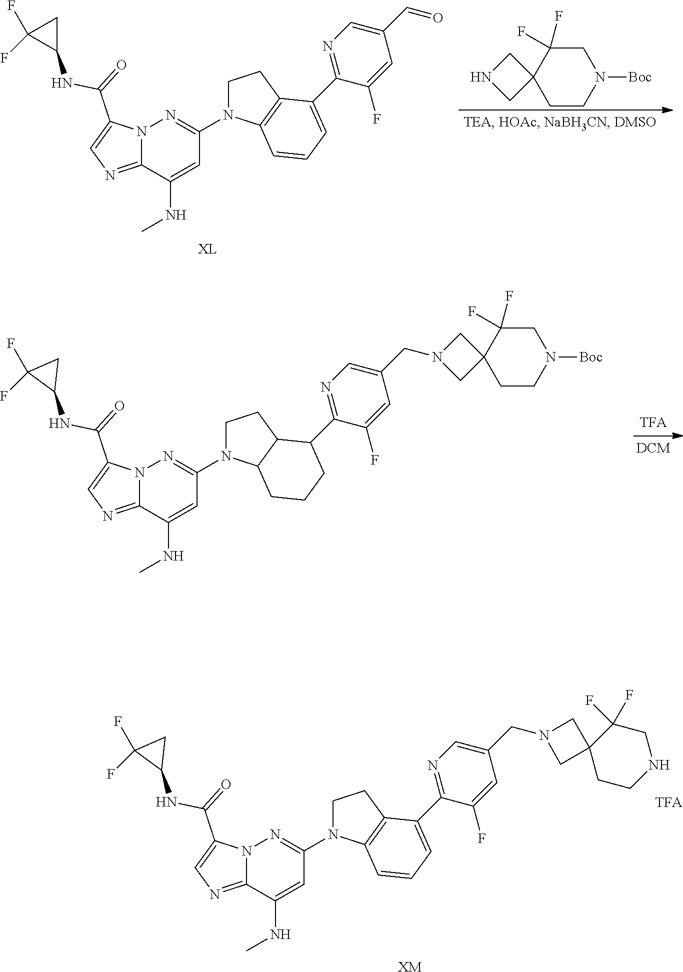

Step 1—Tert-butyl (R)-2-((6-(1-(3-((2,2-difluorocyclopropyl)carbamoyl)-8-(methylamino)imidazo[1,2-b]pyridazin-6-yl)indolin-4-yl)-5-fluoropyridin-3-yl)methyl)-5,5-difluoro-2,7-diazaspiro[3.5]nonane-7-carboxylate. To a stirred solution of tert-butyl 5,5-difluoro-2,7-diazaspiro[3.5]nonane-7-carboxylate (36 mg, 0.14 mmol) and TEA (13 mg, 0.14 mmol) in DMSO (1 mL) were added (R)—N-(2,2-difluorocyclopropyl)-6-(4-(3-fluoro-5-formylpyridin-2-yl)indolin-1-yl)-8-(methylamino)imidazo[1,2-b]pyridazine-3-carboxamide (70 mg, 0.14 mmol, Intermediate XL) and HOAc (24 mg, 0.41 mmol) in turns at rt under nitrogen atmosphere. The resulting mixture was stirred for 1 h at 50° C. under nitrogen atmosphere. To the above mixture was added NaBH₃CN (34 mg, 0.55 mmol) over 2 min at rt. The resulting mixture was stirred for an additional 1 h at 50° C. After completion of the reaction, the reaction mixture was cooled to rt and purified directly by reverse phase flash chromatography (Column: WelFlash™ C18-I, 20-40 μm, 120 g; Eluent A: Water (10 mmol/L NH₄HCO₃); Eluent B: ACN; Gradient: 50%-80% B in 40 min; Flow rate: 60 mL/min; Detector: 220/254 nm; desired fractions were collected at 74% B and concentrated under reduced pressure) to afford the title compound (60 mg, 57% yield) as a yellow solid. LC/MS (ESI, m/z): [(M+1)]⁺=754.2.

Step 2—(R)-6-(4-(5-((5,5-difluoro-2,7-diazaspiro[3.5]nonan-2-yl)methyl)-3-fluoropyridin-2-yl)indolin-1-yl)-N-(2,2-difluorocyclopropyl)-8-(methylamino)imidazo[1,2-b]pyridazine-3-carboxamide trifluoroacetate. To a stirred solution of tert-butyl 2-({6-[1-(3-{[(1R)-2,2-difluorocyclopropyl]carbamoyl}-8-(methylamino)imidazo[1,2-b]pyridazin-6-yl)-2,3-dihydroindol-4-yl]-5-fluoropyridin-3-yl}methyl)-5,5-difluoro-2,7-diazaspiro[3.5]nonane-7-carboxylate (60 mg, 0.080 mmol) in DCM (3 mL) was added TFA (1 mL) dropwise at rt under nitrogen atmosphere. The resulting mixture was stirred for 1 h at rt under nitrogen atmosphere. On completion, the mixture was concentrated under vacuum. The residue was purified by trituration with Et₂O (10 mL) to give the title compound (50 mg, 96% yield) as a yellow solid. LC/MS (ESI, m/z): [(M+1)]⁺=654.2.

### Tert-butyl (R)-7-(1-((benzyloxy)carbonyl)-3,3-difluoropiperidin-4-yl)-2,7-diazaspiro[3.5]nonane-2-carboxylate (Intermediate XN) and tert-butyl (S)-7-(1-((benzyloxy)carbonyl)-3,3-difluoropiperidin-4-yl)-2,7-diazaspiro[3.5]nonane-2-carboxylate (Intermediate XO)

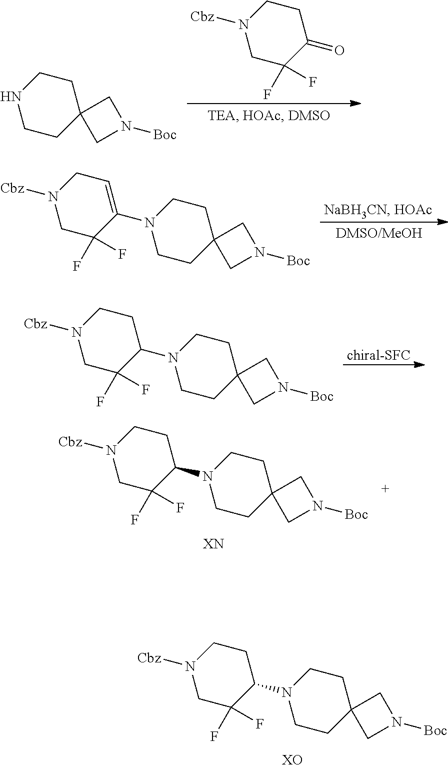

Step 1—Tert-butyl 7-{1-[(benzyloxy)carbonyl]-3,3-difluoropiperidin-4-yl}-2,7-diazaspiro[3.5]nonane-2-carboxylate. To a stirred mixture of tert-butyl 2,7-diazaspiro[3.5]nonane-2-carboxylate (50.00 g, 220.9 mmol) and TEA (92.12 mL, 662.8 mmol) in DMSO (500 mL) were added benzyl 3,3-difluoro-4,4-dihydroxypiperidine-1-carboxylate (63.46 g, 220.9 mmol) and HOAc (63.30 mL, 1.105 mol) at rt under nitrogen atmosphere. The resulting mixture was stirred for 4 h at 100° C. under nitrogen atmosphere. The mixture was allowed to cool down to 50° C. To the above mixture were added MeOH (500 mL). HOAc (50 mL) and NaBH₃CN (41.64 g, 662.78 mmol). The resulting mixture was stirred for an additional 16 h at 50° C. On completion, the mixture was cooled to rt and was purified by reverse phase flash chromatography (Column: WelFlash™ C18-I, 20-40 μm, 320 g; Eluent A: Water (plus 10 mmol/L NH₄HCO₃); Eluent B: ACN; Gradient: 40%-65% B in 15 min; Flow rate: 80 mL/min; Detector: 220/254 nm; desired fractions were collected at 63% B and concentrated under reduced pressure) to afford the title compound (43.00 g, 41% yield) as a white solid. LC/MS (ESI, m/z): [(M+1)]⁺=480.3.

Step 2—Tert-butyl (R)-7-(1-((benzyloxy)carbonyl)-3,3-difluoropiperidin-4-yl)-2,7-diazaspiro[3.5]nonane-2-carboxylate and tert-butyl (S)-7-(1-((benzyloxy)carbonyl)-3,3-difluoropiperidin-4-yl)-2,7-diazaspiro[3.5]nonane-2-carboxylate. Tert-butyl 7-(1-((benzyloxy)carbonyl)-3,3-difluoropiperidin-4-yl)-2,7-diazaspiro[3.5]nonane-2-carboxylate (43 g) was separated by chiral resolution (Column: CHIRALART Cellulose-SC, 7*25 cm, 10 μm; Mobile Phase A: Hex(0.3% DEA)—HPLC, Mobile Phase B: EtOH; Flow rate: 180 mL/min; Gradient: isocratic 15% B; Wave Length: 220 nm; RT1(min): 14; RT2(min): 20; Sample Solvent: MeOH; Injection Volume: 10 mL). The fractions of the faster eluting peak (14 min) were concentrated under vacuum to afford tert-butyl (R)-7-(1-((benzyloxy)carbonyl)-3,3-difluoropiperidin-4-yl)-2,7-diazaspiro[3.5]nonane-2-carboxylate (19 g, 43% yield) as a white solid. The fractions of the slower eluting peak (20 min) were collected and concentrated under vacuum tert-butyl (S)-7-(1-((benzyloxy)carbonyl)-3,3-difluoropiperidin-4-yl)-2,7-diazaspiro[3.5]nonane-2-carboxylate (assumed) (17 g, 35% yield) as a white solid. The absolute configuration of the enantiomers was assigned arbitrarily.

### Tert-butyl (S)-7-(3,3-difluoropiperidin-4-yl)-2,7-diazaspiro[3.5]nonane-2-carboxylate (Intermediate XP)

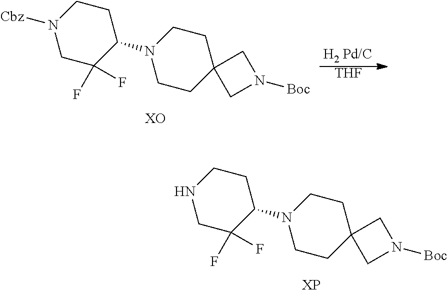 [SMILES: CC(C)(C)OC(=O)N1CC2(CCN([C@H]3CCN(C(=O)OCc4ccccc4)CC3(F)F)CC2)C1 ; CC(C)(C)OC(=O)N1CC2(CCN([C@H]3CCNCC3(F)F)CC2)C1 ; "THF"]

To a solution of tert-butyl (S)-7-(1-((benzyloxy)carbonyl)-3,3-difluoropiperidin-4-yl)-2,7-diazaspiro[3.5]nonane-2-carboxylate (2.1 g, 4.4 mmol, Intermediate XO) in MeOH (50 mL) was added Pd/C (10 wt %, 1.5 g) under nitrogen atmosphere in a 250 mL round-bottom flask. The mixture was hydrogenated at rt for 2 h under hydrogen atmosphere using a hydrogen balloon. Upon completion, the mixture was filtered through a celite pad and concentrated under reduced pressure to give the title compound (1.6 g) as a grey solid. LC/MS (ESI, m/z): [(M+1)]⁺=346.2.

### 6-(4-(((S)-3,3-difluoro-4-(2,7-diazaspiro[3.5]nonan-7-yl)piperidin-1-yl)methyl)indolin-1-yl)-N-((1R,2R)-2-methoxycyclobutyl)-8-(methylamino)imidazo[1,2-b]pyridazine-3-carboxamide (Intermediate XQ)

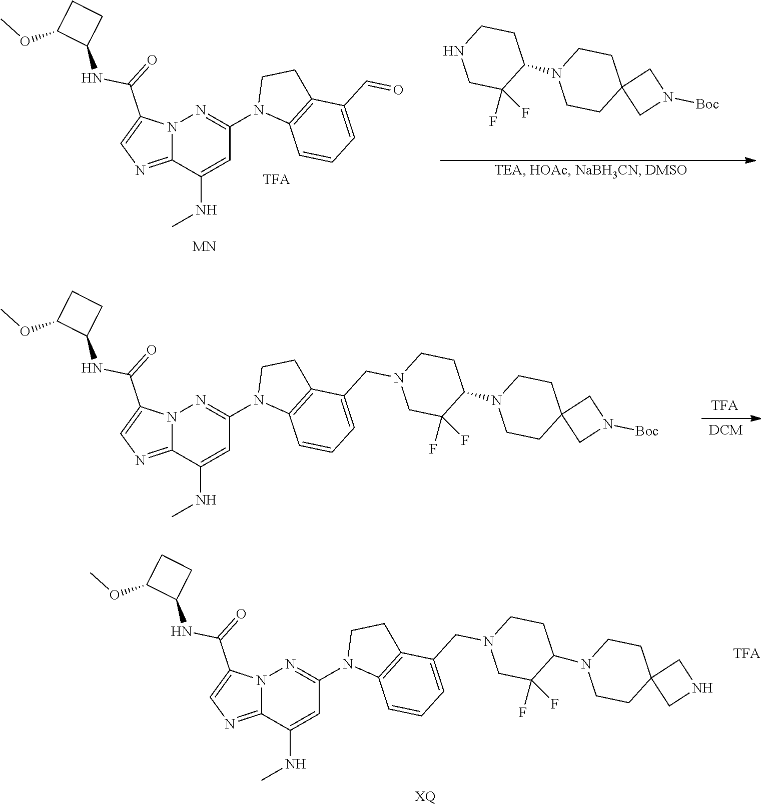

Step 1—Tert-butyl 7-((S)-3,3-difluoro-1-((1-(3-(((1R,2R)-2-methoxycyclobutyl)carbamoyl)-8-(methylamino)imidazo[1,2-b]pyridazin-6-yl)indolin-4-yl)methyl)piperidin-4-yl)-2,7-diazaspiro[3.5]nonane-2-carboxylate. To a stirred solution of tert-butyl (S)-7-(3,3-difluoropiperidin-4-yl)-2,7-diazaspiro[3.5]nonane-2-carboxylate (1.00 g, 2.90 mmol, Intermediate XP) in DMSO (20 mL) was added TEA (878 mg, 8.70 mmol) at rt. The resulting mixture was stirred for 5 min at rt. To the above mixture was added 6-(4-formyl-2,3-dihydroindol-1-yl)-N-[(1R,2R)-2-methoxycyclobutyl]-8-(methylamino)imidazo[1,2-b]pyridazine-3-carboxamide (1.22 g, 2.90 mmol, Intermediate MN) and HOAc (870 mg, 14.49 mmol) at rt. The resulting mixture was stirred for an additional 30 min at 50° C. To the above mixture was added NaBH₃CN (365 mg, 5.80 mmol) at rt. The resulting mixture was stirred for an additional 16 h at 50° C. On completion, the reaction was purified by reverse phase flash chromatography (Column: WelFlash™ C18-I, 20-40 μm, 330 g; Eluent A: Water (plus 10 mmol/L NH₄HCO₃); Eluent B: ACN; Gradient: 65%-75% B in 25 min; Flow rate: 80 mL/min; Detector: 220/254 nm; desired fractions were collected at 70% B and concentrated under reduced pressure) to afford the title compound (910 mg, 42% yield) as a yellow solid. LC/MS (ESI, m/z): [(M+1)]⁺=750.4.

Step 2—6-(4-(((S)-3,3-difluoro-4-(2,7-diazaspiro[3.5]nonan-7-yl)piperidin-1-yl)methyl)indolin-1-yl)-N-((1R,2R)-2-methoxycyclobutyl)-8-(methylamino)imidazo[1,2-b]pyridazine-3-carboxamide 2,2,2-trifluoroacetate. To a stirred solution of tert-butyl 7-((S)-3,3-difluoro-1-((1-(3-(((1R,2R)-2-methoxycyclobutyl)carbamoyl)-8-(methylamino)imidazo[1,2-b]pyridazin-6-yl)indolin-4-yl)methyl)piperidin-4-yl)-2,7-diazaspiro[3.5]nonane-2-carboxylate (910 mg, 1.17 mmol) in DCM (15 mL) was added TFA (5 mL) at rt. The resulting mixture was stirred for 1 h at rt. On completion, the mixture was concentrated under vacuum. The residue was purified by trituration with Et₂O (10 mL) to give the title compound (750 mg, TFA salt) as a yellow solid. LC/MS (ESI, m/z): [(M+1)]=650.4.

### Tert-butyl (R)-7-(3,3-difluoropiperidin-4-yl)-2,7-diazaspiro[3.5]nonane-2-carboxylate (Intermediate XR)

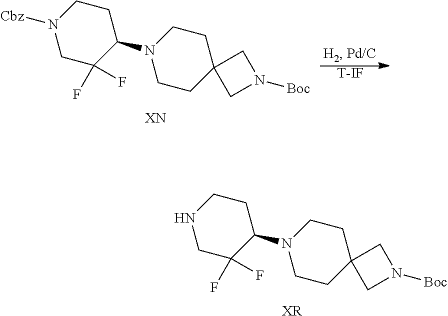

To a solution of tert-butyl (R)-7-(1-((benzyloxy)carbonyl)-3,3-difluoropiperidin-4-yl)-2,7-diazaspiro[3.5]nonane-2-carboxylate (2.5 g, 4.4 mmol, Intermediate XN) in MeOH (50 mL) was added Pd/C (10 wt %, 1.5 g) under nitrogen atmosphere in a 250 mL round-bottom flask. The mixture was hydrogenated at rt for 2 h under hydrogen atmosphere using a hydrogen balloon. On completion, the mixture was filtered through a Celite pad and concentrated under reduced pressure to give the title compound (1.8 g) as a grey solid. LC/MS (ESI, m/z): [(M+1)]⁺=346.2.

### 6-(4-(((R)-3,3-difluoro-4-(2,7-diazaspiro[3.5]nonan-7-yl)piperidin-1-yl)methyl)indolin-1-yl)-N-((1R,2R)-2-methoxycyclobutyl)-8-(methylamino)imidazo[1,2-b]pyridazine-3-carboxamide (Intermediate XS)

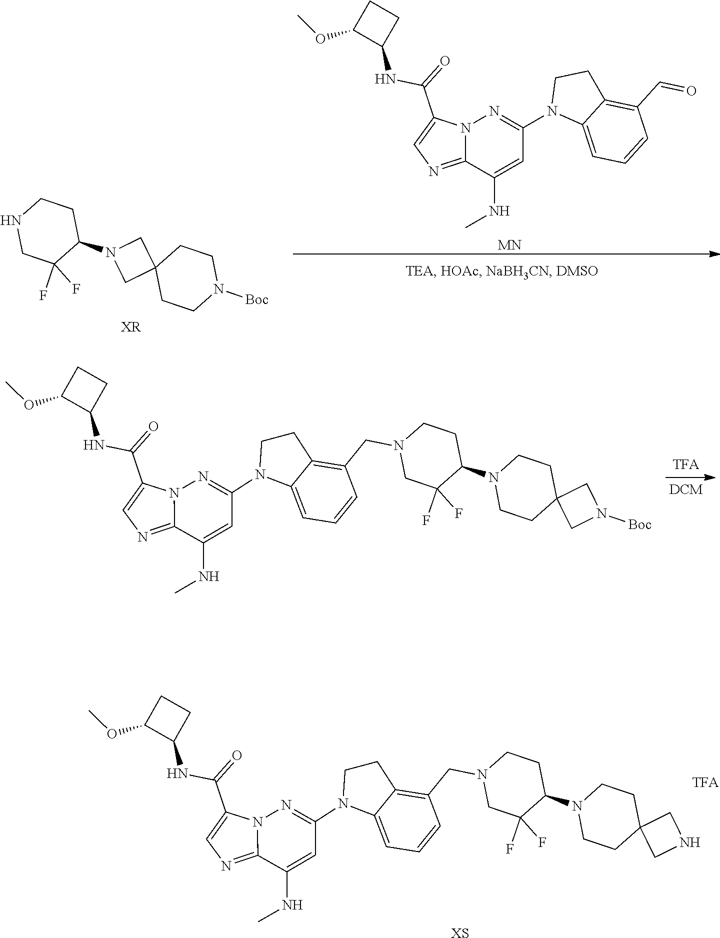

Step 1—Tert-butyl 7-((R)-3,3-difluoro-1-((1-(3-(((1R,2R)-2-methoxycyclobutyl)carbamoyl)-8-(methylamino)imidazo[1,2-b]pyridazin-6-yl)indolin-4-yl)methyl)piperidin-4-yl)-2,7-diazaspiro[3.5]nonane-2-carboxylate. To a stirred mixture of 6-(4-formyl-2,3-dihydroindol-1-yl)-N-[(1R,2R)-2-methoxycyclobutyl]-8-(methylamino)imidazo[1,2-b]pyridazine-3-carboxamide (2.00 g, 4.76 mmol, Intermediate MN) and tert-butyl 7-[(4R)-3,3-difluoropiperidin-4-yl]-2,7-diazaspiro[3.5]nonane-2-carboxylate (1.64 g, 4.76 mmol, Intermediate XR) in DMSO (20 mL) were added Et₃N (0.96 g, 9.51 mmol) and HOAc (2.86 g, 47.57 mmol) at room temperature. The resulting mixture was stirred for 1 h at 50° C. To the above mixture was added NaBH₃CN (1.49 g, 23.78 mmol) at room temperature. The resulting mixture was stirred for additional 1 h at 50° C. The resulting mixture was purified by reversed-phase flash chromatography with the following conditions: Column: WelFlash™ C18-I, 20-40 μm, 330 g; Eluent A: Water (plus 10 mmol/L NH₄HCO₃); Eluent B: ACN; Gradient: 85%-95% B in 15 min; Flow rate: 80 mL/min; Detector: 220/254 nm; desired fractions were collected at 90% B and concentrated under reduced pressure to afford tert-butyl 7-((R)-3,3-difluoro-1-((1-(3-(((1R,2R)-2-methoxycyclobutyl)carbamoyl)-8-(methylamino)imidazo[1,2-b]pyridazin-6-yl)indolin-4-yl)methyl)piperidin-4-yl)-2,7-diazaspiro[3.5]nonane-2-carboxylate (1.8 g, 50%) as a white solid. LC/MS (ESI, m/z): [(M+1)]=750.4.

Step 2—6-(4-(((R)-3,3-difluoro-4-(2,7-diazaspiro[3.5]nonan-7-yl)piperidin-1-yl)methyl)indolin-1-yl)-N-((1R,2R)-2-methoxycyclobutyl)-8-(methylamino)imidazo[1,2-b]pyridazine-3-carboxamide trifluoroacetate. To a stirred mixture of tert-butyl 7-[(4R)-3,3-difluoro-1-{[1-(3-{[(1R,2R)-2-methoxycyclobutyl]carbamoyl}-8-(methylamino)imidazo[1,2-b]pyridazin-6-yl)-2,3-dihydroindol-4-yl]methyl}piperidin-4-yl]-2,7-diazaspiro[3.5]nonane-2-carboxylate (500 mg, 0.667 mmol) in DCM (6 mL) was added TFA (2 mL) at rt. The resulting mixture was stirred for 1 h at rt. On completion, the mixture was concentrated under reduced pressure. The residue was purified by trituration with diethyl ether (20 mL) to give the title compound (520 mg, TFA salt) as a white solid. LC/MS (ESI, m/z): [(M+H)]⁻=650.4.

### 6-Bromo-7-fluoro-1-methyl-3H-1,3-benzodiazol-2-one (Intermediate XT)

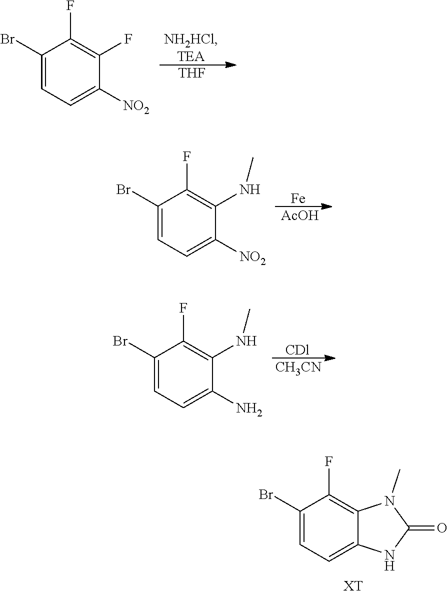

Step 1—3-Bromo-2-fluoro-N-methyl-6-nitroaniline. To a stirred solution of 1-bromo-2,3-difluoro-4-nitrobenzene (25 g, 105 mmol) and dimethylamine hydrochloride (15 g, 180 mmol) in THF (300 mL) were added TEA (43.81 mL, 315.1 mmol) at 0° C. under nitrogen atmosphere. The resulting mixture was stirred for 16 h at 25° C. under nitrogen atmosphere. On completion, the resulting mixture was diluted with water (200 mL) and extracted with EtOAc (3×200 mL). The combined organic layers were dried over anhydrous Na₂SO₄. After filtration, the filtrate was concentrated under reduced pressure. The residue was purified by silica gel column chromatography, eluted with PE/EA (50:1-10:1), to afford the title compound (25 g, 96% yield) as a yellow solid. LC/MS (ESI, m/z): [(M+H)]⁺=249.0, 251.0.

Step 2—5-Bromo-6-fluoro-N1-methylbenzene-1,2-diamine. A solution of 3-bromo-2-fluoro-N-methyl-6-nitroaniline (9 g, 40 mmol) and Fe powder (16.15 g, 289.1 mmol) in HOAc (100 mL) was stirred for 16 h at 25° C. under nitrogen atmosphere. On completion, the mixture was basified to pH 9 with saturated NaHCO₃ (aq.). The resulting mixture was filtered, the filter cake was washed with EtOAc (3×200 mL). The filtrate was concentrated under reduced pressure. The residue was purified by silica gel column chromatography, eluted with PE/EA (1:1), to afford the title compound (5 g, 63% yield) as a brown oil. LC/MS (ESI, m/z): [(M+H)]⁺=218.0, 220.0.

Step 3—6-bromo-7-fluoro-1-methyl-3H-1,3-benzodiazol-2-one. To a stirred solution of 5-bromo-6-fluoro-N1-methylbenzene-1,2-diamine (5 g, 20 mmol) and CDI (5.55 g, 34.2 mmol) in ACN (75 mL) at 25° C. under nitrogen atmosphere. The resulting mixture was stirred at reflux for 16 h under nitrogen atmosphere. On completion, the mixture was cooled to rt and filtered, and the filter cake was washed with MeOH (3×100 mL). The filtrate was concentrated under reduced pressure. The residue was purified by silica gel column chromatography, eluted with PE/EA (1:1), to afford the title compound (3 g, 54% yield) as a brown solid. LC/MS (ESI, m/z): [(M+H)]⁺=245.0, 247.0.

### 1-[(4-Methoxyphenyl)methyl]-2,6-dioxopiperidin-3-yl trifluoromethanesulfonate (Intermediate XU)

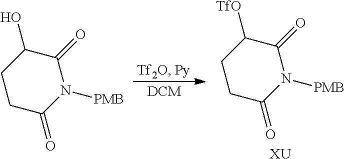

To a solution of 3-hydroxy-1-[(4-methoxyphenyl)methyl]piperidine-2,6-dione ((25 g, 100 mmol, CAS #2357109-89-6) and pyridine (15.87 g, 200.6 mmol) in DCM (250 mL) was added Tf₂O (42.44 g, 150.4 mmol) dropwise at −10° C. The mixture was stirred at −10° C. for 1.5 hrs under N₂ atmosphere. On completion, the mixture was concentrated in vacuo. The residue was purified by column chromatography on silica gel (PE: EA=2:1). The crude product was re-crystallized from hexane to afford the title compound (32 g, 84% yield) as a brown oil. LC/MS (ESI, m/z): [(M+H)]⁺=382.1.

### 1-Benzyl-3,3-difluoro-4,1′:4′,4″-terpiperidine (Intermediate XV)

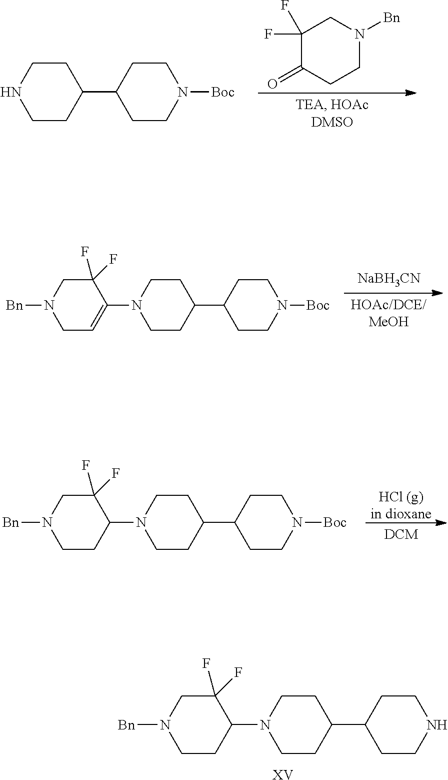

Step 1—Tert-butyl 1′-(1-benzyl-3,3-difluoro-1,2,3,6-tetrahydropyridin-4-yl)-[4,4′-bipiperidine]-1-carboxylate. To a stirred mixture of tert-butyl [4,4′-bipiperidine]-1-carboxylate (5.00 g, 18.6 mmol, CAS #171049-35-7), TEA (1.88 g, 18.6 mmol) and HOAc (3.36 g, 55.89 mmol) in toluene (100 mL) was added 1-benzyl-3,3-difluoropiperidin-4-one (6.29 g, 27.9 mmol) at rt under N₂ atmosphere. The resulting mixture was stirred for 16 h at 110° C. under nitrogen atmosphere. On completion, the mixture was cooled to rt and basified to pH 8 with saturated NaHCO₃ (aq.). The resulting mixture was extracted with EtOAc (3×200 mL). The combined organic layers were washed with water (3×500 mL), and dried over anhydrous Na₂SO₄. After filtration, the filtrate was concentrated under reduced pressure to afford the title compound (8.1 g, 91% yield) as a yellow semi-solid. LC/MS (ESI, m/z): [(M+H)]⁺=476.3.

Step 2—Tert-butyl 1-benzyl-3,3-difluoro-[4,1′:4′,4″-terpiperidine]-1″-carboxylate. To a stirred mixture of tert-butyl 1′-(1-benzyl-3,3-difluoro-1,2,3,6-tetrahydropyridin-4-yl)-[4,4′-bipiperidine]-1-carboxylate (8.80 g, 18.5 mmol) and HOAc (9 mL) in DCE (90 mL) and MeOH (90 mL) was added NaBH₃CN (5.81 g, 92.5 mmol) at rt under N₂ atmosphere. The resulting mixture was stirred for 2 h at rt under nitrogen atmosphere. On completion, the mixture was diluted with water (300 mL) and extracted with EtOAc (3×300 mL). The combined organic layers were washed with brine (2×200 mL), and dried over anhydrous Na₂SO₄. After filtration, the filtrate was concentrated under reduced pressure. The residue was purified by silica gel column chromatography, eluted with PE/EA (3:1), to afford the title compound (4.2 g, 47% yield) as a yellow semi-solid. LC/MS (ESI, m/z): [(M+H)]⁺=478.3.

Step 3—1-Benzyl-3,3-difluoro-4,1′:4′,4″-terpiperidine hydrochloride. To a stirred mixture of tert-butyl 1-benzyl-3,3-difluoro-[4,1′:4′,4″-terpiperidine]-1″-carboxylate (4.20 g, 8.79 mmol) in DCM (15 mL) was added 4 M HCl (gas) in 1,4-dioxane (40 mL) dropwise at 0° C. under N₂ atmosphere. The resulting mixture was stirred for 1 h at rt under nitrogen atmosphere. On completion, the mixture was concentrated under reduced pressure the title compound (3.7 g, 89% yield) as a light yellow solid. LC/MS (ESI, m/z): [(M+H)]⁺=378.3.

### Tert-butyl 3,3-difluoro-4-(piperazin-1-ylmethyl)piperidine-1-carboxylate (Intermediate XW)

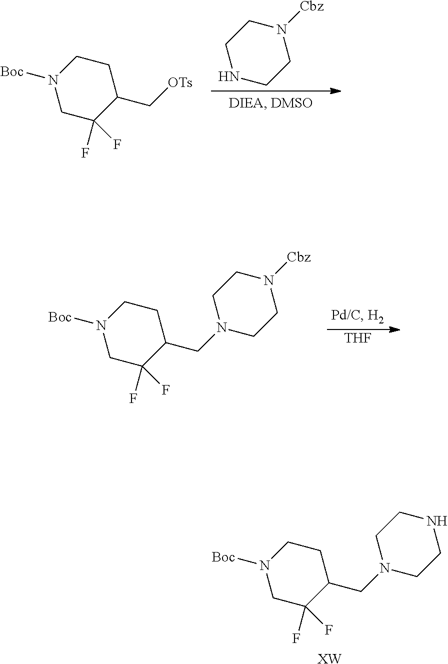

Step 1—Benzyl 4-{[1-(tert-butoxycarbonyl)-3,3-difluoropiperidin-4-yl]methyl}piperazine-1-carboxylate. To a stirred mixture of tert-butyl 3,3-difluoro-4-{[(4-methylbenzenesulfonyl)oxy]methyl}piperidine-1-carboxylate (1 g, 3 mmol, synthesized via Step 1 of Intermediate VV) and benzyl piperazine-1-carboxylate (0.54 g, 2.47 mmol) in DMSO (10 mL) was added DIEA (0.96 g, 7.40 mmol) at rt under nitrogen atmosphere. The resulting mixture was stirred at 100° C. for 16 h under nitrogen atmosphere. On completion, the mixture was cooled to rt and concentrated under reduced pressure. The residue was purified by reverse flash chromatography (column, C18 silica gel; mobile phase A: water (10 mmol/L NH₄HCO₃), mobile phase B: ACN, 30% to 50% gradient in 30 min; detector, UV 254 nm; the fractions were collected at 45% and concentrated under reduced pressure) to afford the title compound (600 mg, 54% yield) as a yellow oil. LC/MS (ESI, m/z): [(M+H)]⁺=454.2.

Step 2—Tert-butyl 3,3-difluoro-4-(piperazin-1-ylmethyl)piperidine-1-carboxylate. To a solution of benzyl 4-{[1-(tert-butoxycarbonyl)-3,3-difluoropiperidin-4-yl]methyl}piperazine-1-carboxylate (600 mg, 1.32 mmol) in THF (8 mL) was added Pd/C (703.94 mg, 6.615 mmol) at rt for 5 min under hydrogen atmosphere. The resulting mixture was stirred at rt for an additional 2 h. On completion, the resulting mixture was filtered, and the filter cake was washed with THF (10×3 mL). The filtrate was concentrated under reduced pressure to afford the title compound (400 mg, 950% yield) as a black oil. LC/MS (ESI, m/z): [(M+H)]f=320.2.

### Indoline-4-carbaldehyde (Intermediate XX)

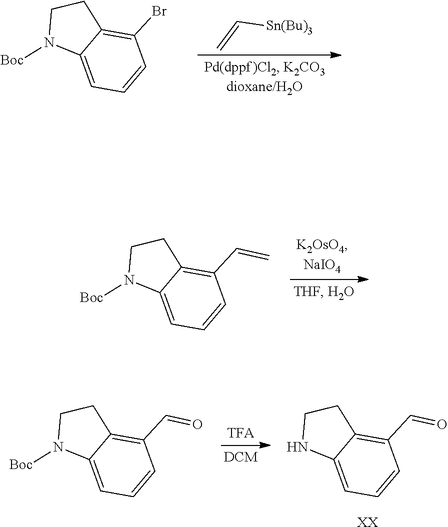

Step 1—Tert-butyl 4-vinylindoline-1-carboxylate. To a stirred mixture of tert-butyl 4-bromoindoline-1-carboxylate (100 g, 340 mmol), tributyl(vinyl)stannane (161 g, 505 mmol) in dioxane (150 mL) was added Pd(dppf)Cl₂·CH₂Cl₂(27.47 g, 33.67 mmol) in portions at rt under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (12:1), to afford the title compound (75 g, 91% yield) as a light yellow solid. ¹H NMR (300 MHz, Chloroform-d) δ 7.82-7.67 (m, 1H), 7.25-7.02 (m, 2H), 6.71 (dd, J=17.6, 11.1 Hz, 1H), 5.71 (dd, J=17.6, 1.1 Hz, 1H), 5.33 (dd, J=11.0, 1.1 Hz, 1H), 4.01 (t, J=8.7 Hz, 2H), 3.11 (t, J=8.7 Hz, 2H), 1.60 (s, 9H).

Step 2—Tert-butyl 4-formylindoline-1-carboxylate. To a solution of tert-butyl 4-vinylindoline-1-carboxylate (75.00 g, 306.1 mmol) in THF (1400 mL) and H₂O (700 mL) was added NaIO₄ (330.61 g, 1.53 mol) in portions for 10 min at rt under nitrogen atmosphere followed by the addition of K₂OsO₄·2H₂O (9.52 g, 30.61 mmol) in portions at 0° C. The resulting mixture was then stirred for 1 h at rt. On completion, the mixture was quenched by sat. aq. Na₂S₂O₃ (300 mL) and extracted with EtOAc (3×500 mL). The combined organic layers were washed with brine (3×1 L), and dried over anhydrous Na₂SO₄. After filtration, the filtrate was concentrated under reduced pressure. The residue was purified by silica gel column chromatography, eluted with PE/EA (10:1), to afford the title compound (45 g, 60% yield) as a light yellow solid. ¹H NMR (300 MHz, Chloroform-d) δ 10.08 (s, 1H), 8.07 (s, 1H), 7.43-7.31 (m, 2H), 4.06 (t, J=8.8 Hz, 2H), 3.47 (t, J=8.8 Hz, 2H), 1.59 (s, 9H). LC/MS (ESI, m/z): [(M+1)]⁺=248.2.

Step 3—Indoline-4-carbaldehyde. A mixture of tert-butyl 4-formylindoline-1-carboxylate (45 g, 180 mmol) in DCM (300 mL) and TFA (100 mL) was stirred for 1 h at rt. On completion, to the mixture was added Na₂CO₃ (aq.) dropwise until the pH was 8. The resulting mixture was diluted with water (500 mL) and extracted with DCM (3×500 mL). The combined organic layers were washed with brine (1×500 mL), and dried over anhydrous Na₂SO₄. After filtration, the filtrate was concentrated under reduced pressure to afford the title compound (17 g, 91% yield) as a brown solid. ¹H NMR (400 MHz, DMSO-d₆) δ 10.07 (s, 1H), 7.25-7.21 (m, 2H), 6.95-6.92 (m, 1H), 3.69 (t, J=8.5 Hz, 2H), 3.44 (t, J=8.5 Hz, 2H). LC/MS (ESI, m/z): [(M+1)]⁺=148.1.

### 2-[1-(2-Bromopyridin-4-yl)piperidin-4-yl]ethanol (Intermediate DS)

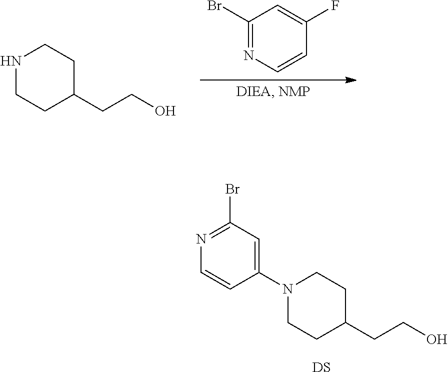

To a stirred solution of 4-piperidine ethanol (2 g, 15 mmol, CAS #622-26-4) and 2-bromo-4-fluoropyridine (4.09 g, 23.2 mmol, CAS #357927-50-5) in NMP was added DIEA (8.09 mL, 46.4 mmol) in portions at rt under air atmosphere. The resulting mixture was stirred for 1 h at 130° C. under air atmosphere. On completion, the solution was cooled to rt and purified by reversed-phase flash chromatography (column, C18 silica gel; mobile phase, MeCN in Water (0.1% TFA), 20% to 50% gradient in 50 min; detector, UV 254 nm) to give the title compound (2.8 g, 63% yield) as a white solid. LC/MS (ESI, m/z): [(M+1)]⁺=285.1.

### 2-(1-{2-[(3aR,7aR)-4-(3-fluorophenyl)-hexahydro-2H-pyrrolo[3,2-b]pyridin-1-yl]pyridin-4-yl}piperidin-4-yl)acetaldehyde (Intermediate DT)

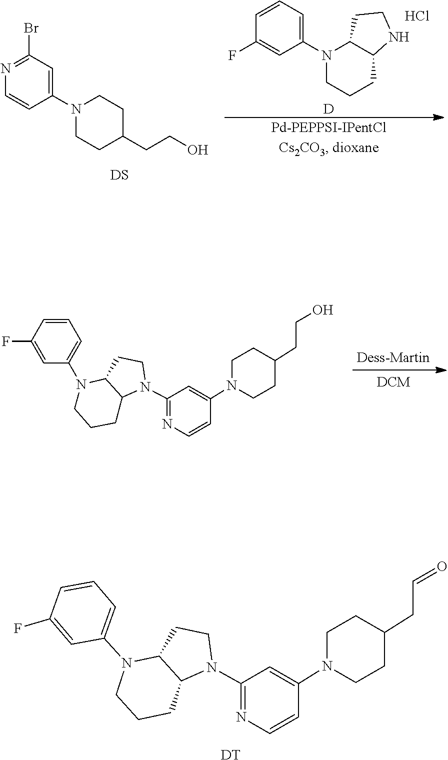

Step 1—2-(1-{2-[(3aR,7aR)-4-(3-fluorophenyl)-hexahydro-2H-pyrrolo[3,2-b]pyridin-1-yl]pyridin-4-yl}piperidin-4-yl)ethanol. To a stirred mixture of (3aR,7aR)-4-(3-fluorophenyl)-octahydropyrrolo[3,2-b]pyridine hydrochloride (500 mg, 1.95 mmol, Intermediate D, prepared as described in PCT/US2023/077741) and 2-[1-(2-bromopyridin-4-yl)piperidin-4-yl]ethanol (555.37 mg, 1.95 mmol, Intermediate DS) in 1,4-dioxane were added {1,3-bis[2,6-bis(pentan-3-yl)phenyl]-4,5-dichloro-2,3-dihydro-1H-imidazol-2-yl}dichloro(2-methyl-1lambda4-pyridin-1-yl)palladium (163.81 mg, 0.195 mmol) and Cs₂CO₃ (296.51 mg, 3.894 mmol) in portions at rt under air atmosphere. The resulting mixture was stirred for 2 h at 100° C. under air atmosphere. On completion, the mixture was cooled to rt and was purified by reversed-phase flash chromatography (column, C18 silica gel; mobile phase, MeCN in Water (10 mmol/L NH₄HCO₃), 30% to 50% gradient in 30 min; detector, UV 254 nm) to give the title compound (580 mg, 70% yield) as a white solid. LC/MS (ESI, m z): [(M+1)]⁺=425.2.

Step 2—2-(1-{2-[(3aR,7aR)-4-(3-fluorophenyl)-hexahydro-2H-pyrrolo[3,2-b]pyridin-1-yl]pyridin-4-yl}piperidin-4-yl)acetaldehyde. To a stirred solution of 2-(1-{2-[(3aR,7aR)-4-(3-fluorophenyl)-hexahydro-2H-pyrrolo[3,2-b]pyridin-1-yl]pyridin-4-yl}piperidin-4-yl)ethanol (200 mg, 0.471 mmol) in DCM (5 mL) was added Dess-Martin periodinane (599.41 mg, 1.413 mmol) dropwise at 0° C. under air atmosphere. The resulting mixture was stirred for 1 h at rt under air atmosphere. On completion, the mixture was concentrated under reduced pressure. The residue was purified by reversed-phase flash chromatography (column, C18 silica gel; mobile phase, MeCN in Water (0.1% TFA), 10% to 50% gradient in 50 min; detector, UV 254 nm) to give the title compound (100 mg, 50% yield) as a black oil. LC/MS (ESI, m/z): [(M+1)]⁺=423.2.

### 6-({5-[(14-Amino-3,6,9,12-tetraoxatetradecan-1-yl)carbamoyl]pyridin-2-yl}amino)-4-{[2-methoxy-3-(pyrimidin-2-yl)phenyl]amino}-N-methylpyridine-3-carboxamide (Intermediate DX)

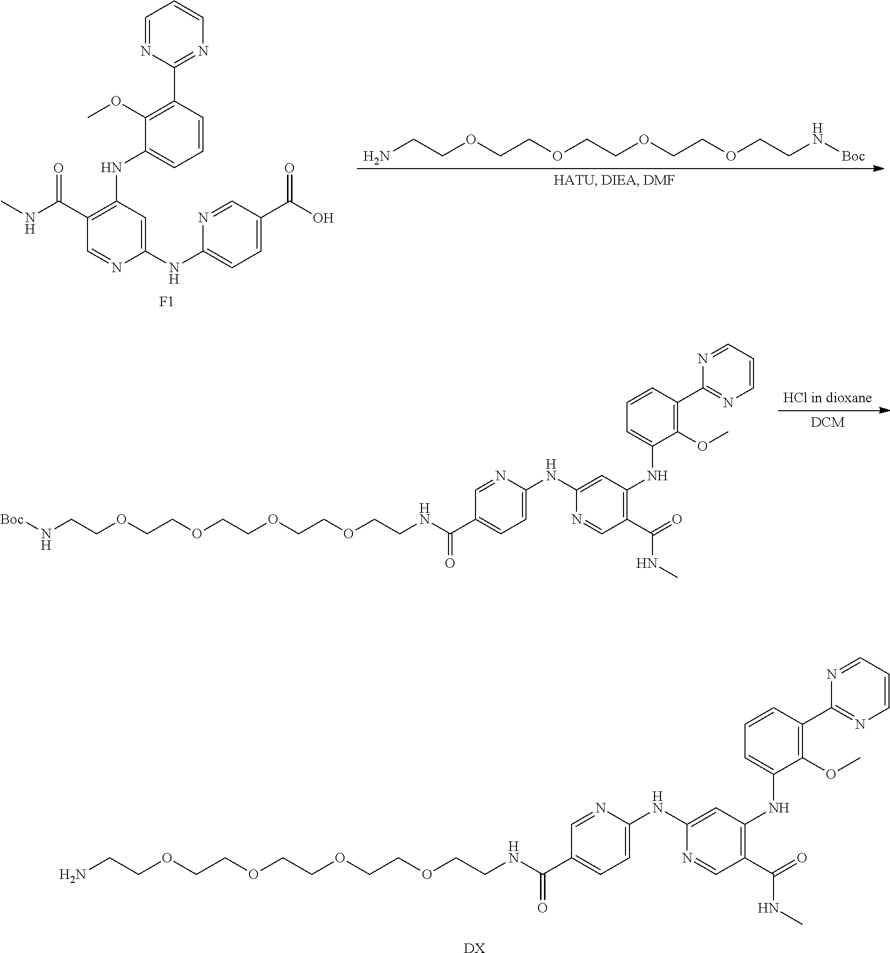

Step 1—Tert-butyl N-[2-[2-[2-[2-[2-[[6-[[4-(2-methoxy-3-pyrimidin-2-yl-anilino)-5-(methylcarbamoyl)-2-pyridyl]amino]pyridine-3-carbonyl]amino]ethoxy]ethoxy]ethoxy]ethoxy]ethyl]carbamate. To a solution of 6-[[4-(2-methoxy-3-pyrimidin-2-yl-anilino)-5-(methylcarbamoyl)-2-pyridyl]amino]pyridine-3-carboxylic acid (3.5 g, 7.4 mmol, Intermediate F1), tert-butyl N-[2-[2-[2-[2-(2-aminoethoxy)ethoxy]ethoxy]ethoxy]ethyl]carbamate (2.62 g, 7.79 mmol, CAS #811442-84-9), and HATU (4.23 g, 11.1 mmol) in DMF (35 mL) was added DIEA (3.84 g, 29.7 mmol, 5.17 mL), then the solution was stirred at 25° C. for 1 h. On completion, the mixture was poured into water (300 mL) and stirred for 5 min. The aqueous phase was extracted with ethyl acetate (100 mL×3). The combined organic phase was washed with brine (50 mL), dried with anhydrous Na₂SO₄, filtered and concentrated in vacuo. The crude product was purified by reverse-phase HPLC (0.1% FA condition) to give the title compound (2.26 g, 35% yield, FA) as a white solid. ¹H NMR (400 MHz, METHANOL-d4) δ=8.93 (d, J=5.0 Hz, 2H), 8.74 (d, J=2.0 Hz, 1H), 8.40 (s, 1H), 8.13 (dd, J=2.4, 8.8 Hz, 1H), 7.69 (dd, J=1.5, 8.0 Hz, 1H), 7.58 (dd, J=1.4, 7.8 Hz, 1H), 7.50 (t, J=5.0 Hz, 1H), 7.44-7.23 (m, 3H), 3.69 (s, 3H), 3.67-3.53 (m, 16H), 3.45 (t, J=5.6 Hz, 2H), 3.17 (t, J=5.6 Hz, 2H), 2.94 (s, 3H), 1.41 (s, 9H).

6-({5-[(14-Amino-3,6,9,12-tetraoxatetradecan-1-yl)carbamoyl]pyridin-2-yl}amino)-4-{[2-methoxy-3-(pyrimidin-2-yl)phenyl]amino}-N-methylpyridine-3-carboxamide. To a stirred solution of tert-butyl N-[14-({6-[(4-{[2-methoxy-3-(pyrimidin-2-yl)phenyl]amino}-5-(methylcarbamoyl)pyridin-2-yl)amino]pyridin-3-yl}formamido)-3,6,9,12-tetraoxatetradecan-1-yl]carbamate (150 mg, 0.190 mmol) in DCM (3 mL) was added HCl (g) (3 mL, 100 mmol) dropwise at rt under air atmosphere. The resulting mixture was stirred for 1 h at rt under air atmosphere. On completion, the mixture was concentrated under reduced pressure to give the title compound (120 mg, HCl salt) as a grey solid. LC/MS (ESI, m z): [(M+1)]⁺=690.3.

### Example 1 (Method 1): Methyl (S)-2-(5-(1-(isoquinolin-4-yl)-N-(1-(6-((4-((2-methoxy-3-(pyrimidin-2-yl)phenyl)amino)-5-(methylcarbamoyl)pyridin-2-yl)amino)pyridin-3-yl)-1-oxo-5,8,11,14-tetraoxa-2-azahexadecan-16-yl)piperidine-3-carboxamido)-2-oxopyridin-1(2H)-yl)acetate (I-912)

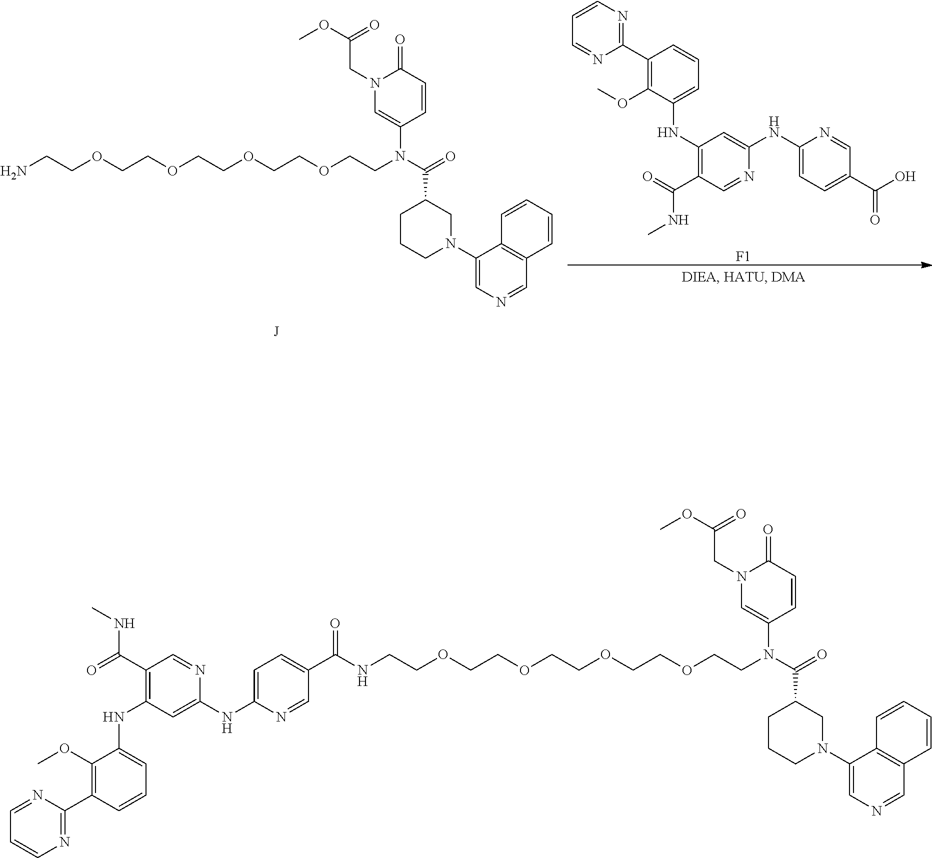 [SMILES: COC(=O)Cn1cc(N(CCOCCOCCOCCOCCN)C(=O)[C@H]2CCCN(c3cncc4ccccc34)C2)ccc1=O ; CNC(=O)c1cnc(Nc2ccc(C(=O)O)cn2)cc1Nc1cccc(-c2ncccn2)c1OC ; "F1" ; CNC(=O)c1cnc(Nc2ccc(C(=O)NCCOCCOCCOCCOCCN(C(=O)[C@H]3CCCN(c4cncc5ccccc45)C3)c3ccc(=O)n(CC(=O)OC)c3)cn2)cc1Nc1cccc(-c2ncccn2)c1OC]

To a stirred solution of 6-[(4-{[2-methoxy-3-(pyrimidin-2-yl)phenyl]amino}-5-(methylcarbamoyl)pyridin-2-yl)amino]pyridine-3-carboxylic acid (88 mg, 0.18 mmol, Intermediate F1) in DMA (3 mL) were added HATU (107 mg, 0.28 mmol) and DIEA (73 mg, 0.56 mmol) at rt under nitrogen atmosphere. The resulting mixture was stirred for 10 min at rt. Next, methyl (S)-2-(5-(N-(14-amino-3,6,9,12-tetraoxatetradecyl)-1-(isoquinolin-4-yl)piperidine-3-carboxamido)-2-oxopyridin-1(2H)-yl)acetate (120 mg, 0.18 mmol, Intermediate J) was added to the reaction mixture, then the mixture was stirred for 1 h at rt. The residue was purified directly without any workup by reverse phase flash chromatography (Column: WelFlash™ C18-I, 20-40 μm, 120 g; Eluent A: Water (10 mmol/L NH₄HCO₃); Eluent B: MeCN; Gradient: 25%-55% B in 40 min; Flow rate: 80 mL/min; Detector: 220/254 nm; desired fractions were collected at 50% B) and concentrated under reduced pressure to afford the title compound (164 mg, 78%) as a white solid. LC/MS (ESI, m/z): [(M+1)]=1093.9.

### Example 2 (Method 1): Synthesis of 6-(4-(5-((7-(4-chloro-3-(2,4-dioxotetrahydropyrimidin-1(2H)-yl)-5-methylbenzoyl)-5,5-difluoro-2,7-diazaspiro[3.5]nonan-2-yl)methyl)-3-fluoropyridin-2-yl)indolin-1-yl)-N-((1R,2S)-2-fluorocyclopropyl)-8-(methylamino)imidazo[1,2-b]pyridazine-3-carboxamide (I-813)

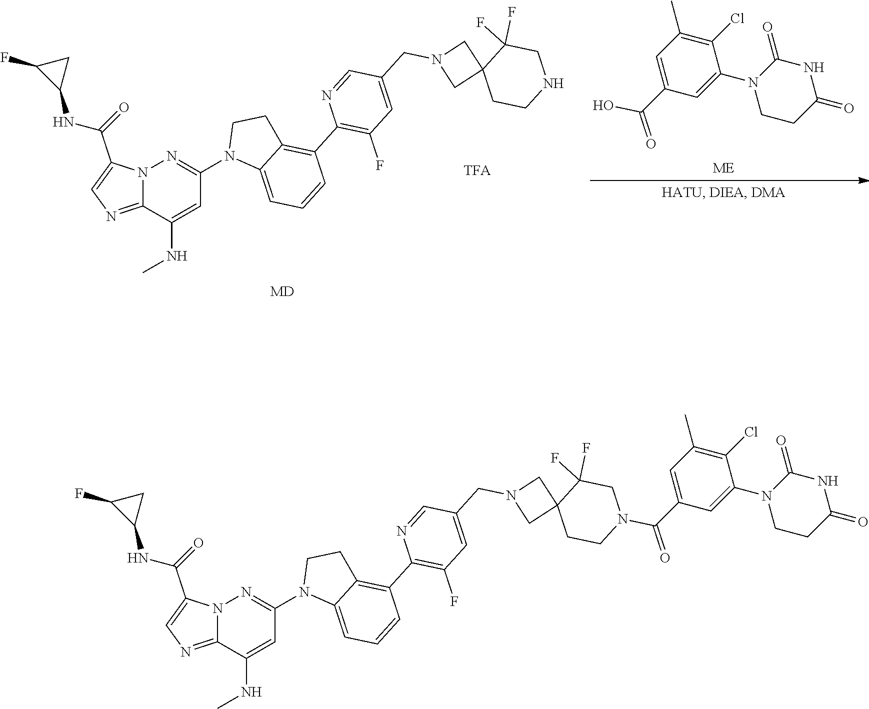

A solution of 4-chloro-3-(2,4-dioxotetrahydropyrimidin-1(2H)-yl)-5-methylbenzoic acid (6.36 g, 22.5 mmol, Intermediate ME) and HATU (11.66 g, 30.67 mmol) in DMA (150 mL) was treated with DIEA (7.93 g, 61.33 mmol) for 10 min at rt. Next, 6-(4-(5-((5,5-difluoro-2,7-diazaspiro[3.5]nonan-2-yl)methyl)-3-fluoropyridin-2-yl)indolin-1-yl)-N-((1R,2S)-2-fluorocyclopropyl)-8-(methylamino)imidazo[1,2-b]pyridazine-3-carboxamide trifluoroacetate (15.00 g, 20.44 mmol, Intermediate MD) was added to the mixture in portions at rt. The resulting mixture was stirred for 1 h at rt. The resulting mixture was purified by reversed-phase flash chromatography (Column: Spherical C18, 20˜40 μm, 330 g; Mobile Phase A: Water (plus 0.1% HCOOH), Mobile Phase B: ACN; Flow rate: 80 mL/min; Gradient (B %): 5%˜30%, 4 min; 30%˜50%, 20 min; 50%˜95%; 2 min; 95%, 5 min; Detector: 254 nm; the fractions containing desired product were collected at 37% B) and concentrated under reduced pressure to afford the title compound (10.98 g, 60(0 yield) as a white solid. ¹H NMR (400 MHz, DMSO-d₆) δ 10.50 (s, 1H), 888-8.83 (m, 1H), 8.48 (s, 1H), 7.96 (s, 1H), 7.85-7.66 (m, 3H), 7.45-7.37 (m, 2H), 7.34-7.24 (m, 1H), 7.10 (d, J=7.6 Hz, 1H), 6.00 (s, 1H), 5.06-4.73 (m, 1H), 4.14 (t, J=8.4 Hz, 2H), 4.01-3.84 (m, 1H), 3.82-3.55 (m, 6H), 3.45-3.39 (m, 2H), 3.27-3.13 (m, 5H), 3.10-3.02 (m, 1H), 2.96 (d, J=4.8 Hz, 3H), 2.83-2.70 (m, 2H-1), 2.42 (s, 3H), 2.15-1.96 (m, 2H), 1.35-1.14 (m, 1H), 1.10-0.89 (m, 1H); LC/MS (ESI, 85/z): [(M+1)]⁺=900.3.

TABLE 2
Compounds synthesized via Method 1, coupling the corresponding amines and carboxylic acids.

| | | | LCMS | |
|---|---|---|---|---|
| | | | (ESI+) | |
| | | | m/z | |
| I-#^{a} | Amine | Acid | (M + H)⁺ | ¹H NMR (400 MHz, DMSO-d6) |
| I-913 | O | F1 | 1042.2 | 10.71 (s, 1H), 10.08 (s, 1H), 9.02-8.91 (m, 3H), 8.65 (d, J = 2.4 Hz, 1H), |
| | | | | 8.59-8.50 (m, 2H), 8.41-8.31 (m, 1H), 8.13 (s, 1H), 8.11-8.03 (m, 2H), |
| | | | | 7.98 (s, 1H), 7.92 (s, 1H), 7.84-7.62 (m, 4H), 7.59-7.49 (m, 3H), 7.43 |
| | | | | (d, J = 7.6 Hz, 1H), 7.39-7.28 (m, 1H), 6.54 (d, J = 9.6 Hz, 1H), 4.90- |
| | | | | 4.59 (m, 2H), 3.78-3.49 (m, 7H), 3.30-3.15 (m, 4H), 2.95-2.69 (m, 6H), |
| | | | | 1.92-1.73 (m, 2H), 1.71-1.57 (m, 2H), 1.56-1.46 (m, 2H), 1.45-1.36 (m, |
| | | | | 2H), 1.35-1.07 (m, 15H) |
| I-914 | R | F1 | 1085.5 | 10.72 (d, J = 2.4 Hz, 1H), 10.08 (s, 1H), 9.05-8.92 (m, 3H), 8.65 (d, |
| | | | | J = 2.3 Hz, 1H), 8.59-8.50 (m, 2H), 8.36 (t, J = 5.4 Hz, 1H), 8.13 (s, |
| | | | | 1H), 8.08-8.04 (m, 2H), 7.99 (s, 1H), 7.92 (s, 1H), 7.84-7.61 (m, 5H), |
| | | | | 7.58-7.49 (m, 2H), 7.43 (dd, J = 7.8, 1.5 Hz, 1H), 7.33 (t, J = 7.8 Hz, |
| | | | | 1H), 6.54 (d, J = 9.7 Hz, 1H), 4.88-4.64 (m, 2H), 3.72 (s, 3H), 3.67-3.50 |
| | | | | (m, 4H), 3.32-3.19 (m, 5H), 2.98-2.69 (m, 6H), 1.95-1.73 (m, 2H), |
| | | | | 1.73-1.56 (m, 2H), 1.50 (t, J = 6.9 Hz, 2H), 1.40 (t, J = 7.2 Hz, 2H), |
| | | | | 1.32-1.11 (m, 20H) |
| I-809 | ML | MM | 946.4 | 10.53 (s, 1H), 8.85 (d, J = 8.8 Hz, 1H), 7.93 (s, 1H), 7.77-7.51 (m, |
| | | | | 4H), 7.39 (d, J = 8.4 Hz, 1H), 6.65 (d, J = 10.0 Hz, 1H), 5.90 (s, 1H), |
| | | | | 4.38-4.11 (m, 3H), 3.89-3.71 (m, 2H), 3.70-3.44 (m, 5H), 3.32- |
| | | | | 3.26 (m, 2H), 3.25-3.08 (m, 5H), 3.04-2.82 (m, 5H), 2.81-2.63 (m, |
| | | | | 7H), 2.42-1.93 (m, 4H), 1.90-1.63 (m, 2H), 1.60-1.30 (m, 10H) |
| I-793 | MO | ME | 942.2 | 10.48 (s, 1H), 9.03 (d, J = 8.8 Hz, 1H), 7.91 (s, 1H), 7.75-7.64 (m, |
| | | | | 2H), 7.45-7.34 (m, 2H), 7.16 (t, J = 8.0 Hz, 1H), 6.86 (d, J = 7.6 Hz, |
| | | | | 1H), 5.96-5.89 (m, 1H), 4.41-4.28 (m, 1H), 4.19 (t, J = 8.4 Hz, 2H), |
| | | | | 3.82-3.68 (m, 2H), 3.66-3.44 (m, 5H), 3.31-3.26 (m, 2H), 3.25- |
| | | | | 3.15 (m, 5H), 3.01-2.84 (m, 5H), 2.83-2.61 (m, 7H), 2.40 (s, 3H), |
| | | | | 2.37-2.22 (m, 1H), 2.19-2.00 (m, 3H), 1.87-1.67 (m, 2H), 1.62- |
| | | | | 1.31 (m, 10H) |
| I-1105 | MH | ME | 926.4 | 10.49 (s, 1H), 9.02 (d, J = 8.7 Hz, 1H), 8.51-8.46 (m, 1H), 7.97-7.87 (m, |
| | | | | 2H), 7.76 (d, J = 11.2 Hz, 1H), 7.72-7.65 (m, 1H), 7.40 (s, 2H), 7.32 (t, |
| | | | | J = 7.9 Hz, 1H), 7.12 (dd, J = 7.6, 2.0 Hz, 1H), 5.97 (s, 1H), 4.42-4.29 |
| | | | | (m, 1H), 4.20 (t, J = 8.5 Hz, 2H), 3.97-3.85 (m, 1H), 3.82-3.69 (m, 5H), |
| | | | | 3.68-3.57 (m, 2H), 3.45-3.39 (m, 1H), 3.38-3.33 (m, 2H), 3.25-3.17 (m, |
| | | | | 7H), 2.97 (d, J = 4.9 Hz, 3H), 2.82-2.68 (m, 2H), 2.42 (s, 3H), 2.15-2.00 |
| | | | | (m, 4H), 1.57-1.38 (m, 2H) |
| I-1079 | MR | ME | 942.5 | 10.48 (s, 1H), 9.02 (d, J = 8.8 Hz, 1H), 7.91 (s, 1H), 7.74-7.59 (m, |
| | | | | 2H), 7.46-7.33 (m, 2H), 7.17 (t, J = 7.6 Hz, 1H), 6.87 (d, J = 7.6 Hz, |
| | | | | 1H), 5.94 (d, J = 3.6 Hz, 1H), 4.42-4.28 (m, 1H), 4.19 (t, J = 8.4 Hz, |
| | | | | 2H), 3.81-3.68 (m, 2H), 3.66-3.46 (m, 5H), 3.43-3.35 (m, 2H), 3.25 |
| | | | | 3.15 (m, 5H), 2.99-2.85 (m, 5H), 2.83-2.59 (m, 7H), 2.40 (s, 3H), |
| | | | | 2.36-2.21 (m, 1H), 2.20-1.98 (m, 3H), 1.88-1.67 (m, 2H), 1.60- |
| | | | | 1.30 (m, 10H) |
| I-1073 | NT | ME | 914.4 | 10.48 (s, 1H), 9.08-8.99 (m, 1H), 7.91 (s, 1H), 7.75-7.63 (m, 2H), 7.38 |
| | | | | (s, 2H), 7.21-7.10 (m, 1H), 6.91-6.85 (m, 1H), 5.95 (s, 1H), 4.41-4.31 |
| | | | | (m, 1H), 4.19 (t, J = 8.6 Hz, 2H), 3.84-3.69 (m, 2H), 3.65-3.45 (m, 6H), |
| | | | | 3.24-3.16 (m, 6H), 3.13-3.01 (m, 4H), 2.96 (d, J = 4.9 Hz, 3H), 2.87- |
| | | | | 2.67 (m, 4H), 2.66-2.57 (m, 2H), 2.41 (s, 3H), 2.36-2.26 (m, 1H), 2.16- |
| | | | | 2.00 (m, 2H), 1.78-1.61 (m, 5H), 1.58-1.37 (m, 3H) |
| I-1069 | MW | MM | 942.4 | 10.51 (s, 1H), 9.03 (d, J = 8.8 Hz, 1H), 7.91 (s, 1H), 7.74-7.60 (m, 3H), |
| | | | | 7.54 (d, J = 2.0 Hz, 1H), 7.38 (dd, J = 8.2, 2.1 Hz, 1H), 7.17 (t, J = 7.8 |
| | | | | Hz, 1H), 6.87 (d, J = 7.6 Hz, 1H), 5.95 (s, 1H), 4.54-4.42 (m, 1H), 4.41- |
| | | | | 4.28 (m, 1H), 4.20 (t, J = 8.6 Hz, 2H), 3.82-3.72 (m, 2H), 3.65-3.55 (m, |
| | | | | 2H), 3.53 (d, J = 5.9 Hz, 2H), 3.25-3.20 (m, 2H), 3.20 (s, 3H), 3.05-2.87 |
| | | | | (m, 8H), 2.85-2.65 (m, 4H), 2.49-2.39 (m, 2H), 2.36-2.22 (m, 1H), 2.19- |
| | | | | 2.01 (m, 3H), 1.84-1.68 (m, 3H), 1.66-1.55 (m, 3H), 1.53-1.39 (m, 2H), |
| | | | | 1.39-1.27 (m, 1H), 1.19-1.02 (m, 5H) |
| I-1068 | NT | MS | 914.4 | 10.45 (s, 1H), 9.06-8.98 (m, 1H), 7.91 (s, 1H), 7.76-7.63 (m, 2H), 7.47- |
| | | | | 7.42 (m, 1H), 7.39-7.28 (m, 1H), 7.21-7.03 (m, 1H), 6.91-6.85 (m, 1H), |
| | | | | 5.94 (s, 1H), 4.40-4.29 (m, 1H), 4.26-4.00 (m, 5H), 3.88-3.74 (m, 2H), |
| | | | | 3.66-3.47 (m, 5H), 3.45-3.34 (m, 3H), 3.25-3.17 (m, 6H), 3.12-2.91 (m, |
| | | | | 5H), 2.89-2.75 (m, 1H), 2.74-2.65 (m, 1H), 2.27-2.20 (m, 4H), 2.15- |
| | | | | 2.00 (m, 3H), 2.00-1.60 (m, 5H), 1.59-1.21 (m, 3H) |
| I-1066 | MW | ME | 956.4 | 10.48 (s, 1H), 9.03 (d, J = 8.7 Hz, 1H), 7.91 (s, 1H), 7.74-7.63 (m, 2H), |
| | | | | 7.40-7.34 (m, 2H), 7.17 (t, J = 7.8 Hz, 1H), 6.87 (d, J = 7.6 Hz, 1H), |
| | | | | 5.95 (s, 1H), 4.55-4.44 (m, 1H), 4.40-4.28 (m, 1H), 4.20 (t, J = 8.6 Hz, |
| | | | | 2H), 3.82-3.68 (m, 2H), 3.65-3.46 (m, 4H), 3.25-3.17 (m, 5H), 3.04- |
| | | | | 2.85 (m, 8H), 2.84-2.66 (m, 4H), 2.49-2.43 (m, 2H), 2.40 (s, 3H), 2.35- |
| | | | | 2.22 (m, 1H), 2.19-2.00 (m, 3H), 1.86-1.69 (m, 3H), 1.67-1.55 (m, 3H), |
| | | | | 1.54-1.39 (m, 2H), 1.37-1.26 (m, 1H), 1.19-1.01 (m, 5H) |
| I-1065 | MZ | ME | 944.3 | 10.51 (s, 1H), 8.86 (d, J = 8.4 Hz, 1H), 8.53-8.41 (m, 1H), 7.95 (s, |
| | | | | 1H), 7.91-7.67 (m, 3H), 7.49-7.33 (m, 2H), 7.00-6.80 (m, 1H), 5.93 |
| | | | | (d, J = 2.8 Hz, 1H), 4.44-4.13 (m, 3H), 3.98-3.84 (m, 1H), 3.84- |
| | | | | 3.55 (m, 7H), 3.46-3.38 (m, 3H), 3.26-3.10 (m, 7H), 2.96 (d, J = 4.8 |
| | | | | Hz, 3H), 2.83-2.65 (m, 2H), 2.42 (s, 3H), 2.15-1.93 (m, 4H), 1.58- |
| | | | | 1.37 (m, 2H) |
| I-1061 | NA | ME | 942.4 | 10.49 (s, 1H), 9.03 (d, J = 8.8 Hz, 1H), 7.91 (s, 1H), 7.74-7.63 (m, |
| | | | | 2H), 7.42-7.35 (m, 2H), 7.16 (t, J = 7.6 Hz, 1H), 6.86 (d, J = 7.6 Hz, |
| | | | | 1H), 5.93 (s, 1H), 4.40-4.29 (m, 1H), 4.19 (t, J = 8.4 Hz, 2H), 3.82- |
| | | | | 3.68 (m, 2H), 3.65-3.45 (m, 5H), 3.31-3.26 (m, 2H), 3.25-3.16 (m, |
| | | | | 5H), 2.99-2.85 (m, 5H), 2.84-2.61 (m, 7H), 2.40 (s, 3H), 2.36-2.22 |
| | | | | (m, 1H), 2.20-1.98 (m, 3H), 1.87-1.69 (m, 2H), 1.61-1.32 (m, 10H) |
| I-1055 | MH | MS | 926.3 | 10.48 (s, 1H), 9.03 (d, J = 8.8 Hz, 1H), 8.51-8.46 (m, 1H), 7.95-7.88 (m, |
| | | | | 2H), 7.80-7.69 (m, 2H), 7.46-7.41 (m, 1H), 7.40-7.36 (m, 1H), 7.32 (t, |
| | | | | J = 7.9 Hz, 1H), 7.15-7.08 (m, 1H), 5.97 (s, 1H), 4.38-4.32 (m, 1H), |
| | | | | 4.20 (t, J = 8.5 Hz, 2H), 3.93-3.71 (m, 5H), 3.69-3.62 (m, 1H), 3.61- |
| | | | | 3.52 (m, 1H), 3.32-3.28 (m, 4H), 3.25-3.14 (m, 7H), 2.96 (d, J = 4.9 Hz, |
| | | | | 3H), 2.89-2.76 (m, 1H), 2.73-2.65 (m, 1H), 2.25 (s, 3H), 2.16-2.00 (m, |
| | | | | 4H), 1.56-1.40 (m, 2H) |
| I-1043 | NA | MS | 942.4 | 10.45 (s, 1H), 9.03 (d, J = 8.8 Hz, 1H), 7.91 (s, 1H), 7.77-7.61 (m, |
| | | | | 2H), 7.44 (d, J = 1.6 Hz, 1H), 7.36 (d, J = 1.6 Hz, 1H), 7.17 (t, J = 7.6 |
| | | | | Hz, 1H), 6.87 (d, J = 7.6 Hz, 1H), 5.95 (s, 1H), 4.40-4.28 (m, 1H), 4.20 |
| | | | | (t, J = 8.4 Hz, 2H), 3.89-3.72 (m, 2H), 3.62-3.46 (m, 5H), 3.30-3.26 |
| | | | | (m, 3H), 3.25-3.16 (m, 5H), 2.96 (d, J = 4.8 Hz, 3H), 2.95-2.77 (m, |
| | | | | 3H), 2.75-2.62 (m, 5H), 2.38-2.21 (m, 4H), 2.19-2.00 (m, 3H), 1.86- |
| | | | | 1.69 (m, 2H), 1.61-1.32 (m, 10H) |
| I-1041 | NC | ME | 914.4 | 10.50 (s, 1H), 9.03 (d, J = 8.7 Hz, 1H), 7.91 (s, 1H), 7.74-7.66 (m, 2H), |
| | | | | 7.66-7.58 (m, 2H), 7.17 (t, J = 7.8 Hz, 1H), 6.87 (d, J = 7.6 Hz, 1H), |
| | | | | 5.95 (s, 1H), 4.38-4.28 (m, 1H), 4.20 (t, J = 8.5 Hz, 2H), 4.02 (s, 2H), |
| | | | | 3.80-3.69 (m, 4H), 3.62-3.51 (m, 3H), 3.25-3.17 (m, 5H), 2.96 (d, J = |
| | | | | 4.9 Hz, 3H), 2.92-2.81 (m, 2H), 2.80-2.70 (m, 3H), 2.69-2.59 (m, 4H), |
| | | | | 2.42 (s, 3H), 2.33-2.22 (m, 1H), 2.17-2.01 (m, 3H), 1.85-1.62 (m, 6H), |
| | | | | 1.59-1.49 (m, 1H), 1.47-1.37 (m, 1H) |
| I-1040 | NC | MS | 914.4 | 10.47 (s, 1H), 9.03 (d, J = 8.8 Hz, 1H), 7.91 (s, 1H), 7.74-7.63 (m, 3H), |
| | | | | 7.57 (d, J = 1.6 Hz, 1H), 7.17 (t, J = 7.8 Hz, 1H), 6.87 (d, J = 7.6 Hz, |
| | | | | 1H), 5.95 (s, 1H), 4.40-4.27 (m, 1H), 4.20 (t, J = 8.5 Hz, 2H), 4.02 (s, |
| | | | | 2H), 3.91-3.81 (m, 1H), 3.81-3.69 (m, 3H), 3.59-3.47 (m, 3H), 3.25- |
| | | | | 3.15 (m, 5H), 2.96 (d, J = 4.9 Hz, 3H), 2.93-2.78 (m, 3H), 2.77-2.57 (m, |
| | | | | 6H), 2.36-2.27 (m, 1H), 2.25 (s, 3H), 2.20-2.00 (m, 3H), 1.86-1.63 (m, |
| | | | | 6H), 1.57-1.40 (m, 2H) |
| I-1039 | MW | MS | 956.4 | 10.46 (s, 1H), 9.03 (d, J = 8.7 Hz, 1H), 7.91 (s, 1H), 7.74-7.64 (m, 2H), |
| | | | | 7.43 (d, J = 1.6 Hz, 1H), 7.35 (d, J = 1.7 Hz, 1H), 7.17 (t, J = 7.8 Hz, |
| | | | | 1H), 6.87 (d, J = 7.6 Hz, 1H), 5.95 (s, 1H), 4.52-4.44 (m, 1H), 4.39-4.29 |
| | | | | (m, 1H), 4.20 (t, J = 8.5 Hz, 2H), 3.91-3.72 (m, 2H), 3.62-3.49 (m, 4H), |
| | | | | 3.25-3.21 (m, 2H), 3.19 (s, 3H), 3.05-2.86 (m, 8H), 2.84-2.78 (m, 1H), |
| | | | | 2.76-2.63 (m, 3H), 2.48-2.39 (m, 2H), 2.35-2.25 (m, 1H), 2.23 (s, 3H), |
| | | | | 2.18-2.02 (m, 3H), 1.85-1.69 (m, 3H), 1.67-1.55 (m, 3H), 1.54-1.40 (m, |
| | | | | 2H), 1.37-1.27 (m, 1H), 1.20-0.99 (m, 5H) |
| I-1038 | MZ | MS | 944.2 | 10.47 (s, 1H), 8.86 (d, J = 8.8 Hz, 1H), 8.49 (d, J = 1.6 Hz, 1H), 7.95 (s, |
| | | | | 1H), 7.87-7.69 (m, 3H), 7.48-7.34 (m, 2H), 6.95-6.86 (m, 1H), 5.94 |
| | | | | (s, 1H), 4.42-4.29 (m, 1H), 4.23 (t, J = 8.4 Hz, 2H), 3.95-3.71 (m, |
| | | | | 5H), 3.71-3.51 (m, 3H), 3.48-3.36 (m, 3H), 3.27-3.13 (m, 7H), 2.96 |
| | | | | (d, J = 4.8 Hz, 3H), 2.90-2.77 (m, 1H), 2.77-2.64 (m, 1H), 2.25 (s, |
| | | | | 3H), 2.15-1.94 (m, 4H), 1.58-1.39 (m, 2H) |
| I-1037 | NE | ME | 970.5 | 10.46 (s, 1H), 9.02 (d, J = 8.7 Hz, 1H), 7.91 (s, 1H), 7.71-7.63 (m, 2H), |
| | | | | 7.37 (s, 2H), 7.17 (t, J = 7.8 Hz, 1H), 6.87 (d, J = 7.5 Hz, 1H), 5.95 (d, |
| | | | | J = 2.0 Hz, 1H), 4.47-4.39 (m, 1H), 4.34 (p, J = 8.6 Hz, 1H), 4.19 (t, |
| | | | | J = 8.6 Hz, 2H), 3.82-3.67 (m, 2H), 3.66-3.46 (m, 4H), 3.24-3.18 (m, |
| | | | | 5H), 3.04-2.83 (m, 8H), 2.83-2.69 (m, 4H), 2.48-2.43 (m, 2H), 2.40 (s, |
| | | | | 3H), 2.35-2.22 (m, 1H), 2.19-1.99 (m, 3H), 1.83-1.37 (m, 9H), 1.34- |
| | | | | 1.26 (m, 1H), 1.16-0.98 (m, 6H) |
| I-1035 | NX | ME | 932.3 | 10.49 (s, 1H), 9.11 (d, J = 8.4 Hz, 1H), 8.49 (s, 1H), 7.99 (s, 1H), 7.84 |
| | | | | (d, J = 8.0 Hz, 1H), 7.80-7.70 (m, 2H), 7.43-7.37 (m, 2H), 7.31 (t, |
| | | | | J = 8.0 Hz, 1H), 7.17-7.07 (m, 1H), 6.02 (s, 1H), 5.14-4.98 (m, 1H), |
| | | | | 4.27-4.10 (m, 2H), 3.99-3.82 (m, 1H), 3.81-3.58 (m, 6H), 3.46- |
| | | | | 3.37 (m, 3H), 3.27-3.15 (m, 4H), 2.98 (d, J = 4.8 Hz, 3H), 2.83-2.70 |
| | | | | (m, 2H), 2.47-2.38 (m, 5H), 2.37-2.24 (m, 1H), 2.11-1.98 (m, 2H), |
| | | | | 1.78-1.64 (m, 1H) |
| I-1033 | NE | MS | 970.4 | 10.48 (s, 1H), 9.03 (d, J = 8.8 Hz, 1H), 7.91 (d, J = 1.3 Hz, 1H), 7.79 (s, |
| | | | | 1H), 7.73-7.66 (m, 2H), 7.37 (s, 2H), 7.17 (t, J = 7.8 Hz, 1H), 6.86 (d, |
| | | | | J = 7.5 Hz, 1H), 5.94 (s, 1H), 4.45-4.40 (m, 1H), 4.34 (p, J = 8.6 Hz, 1H), |
| | | | | 4.19 (t, J = 8.3 Hz, 2H), 3.82-3.67 (m, 2H), 3.66-3.45 (m, 4H), 3.24- |
| | | | | 3.15 (m, 5H), 3.09-2.83 (m, 8H), 2.79-2.66 (m, 4H), 2.48-2.42 (m, 2H), |
| | | | | 2.40 (s, 3H), 2.35-2.24 (m, 1H), 2.19-1.99 (m, 3H), 1.83-1.38 (m, 9H), |
| | | | | 1.33-1.23 (s, 1H), 1.15-0.98 (m, 6H). |
| I-1028 | NZ | MS | 914.3 | 10.47 (s, 1H), 9.04 (d, J = 8.4 Hz, 1H), 8.48 (d, J = 1.6 Hz, 1H), 7.98- |
| | | | | 7.91 (m, 2H), 7.81-7.64 (m, 2H), 7.48-7.36 (m, 2H), 7.31 (t, J = 8.0 |
| | | | | Hz, 1H), 7.11 (dd, J = 7.6, 2.0 Hz, 1H), 5.95 (s, 1H), 5.06-4.80 (m, |
| | | | | 1H), 4.66-4.49 (m, 1H), 4.19 (t, J = 8.4 Hz, 2H), 3.96-3.79 (m, 2H), |
| | | | | 3.78-3.50 (m, 5H), 3.47-3.39 (m, 3H), 3.27-3.15 (m, 4H), 2.96 (d, |
| | | | | J = 4.8 Hz, 3H), 2.90-2.77 (m, 1H), 2.76-2.63 (m, 1H), 2.25 (s, 3H), |
| | | | | 2.23-2.11 (m, 2H), 2.11-1.99 (m, 2H), 1.91-1.75 (m, 1H), 1.50- |
| | | | | 1.35 (m, 1H) |
| I-1027 | NZ | ME | 914.3 | 10.52 (s, 1H), 9.04 (d, J = 8.4 Hz, 1H), 8.48 (d, J = 2.0 Hz, 1H), 8.18 (s, |
| | | | | 1H), 8.04-7.87 (m, 2H), 7.85-7.66 (m, 2H), 7.45-7.36 (m, 2H), 7.31 |
| | | | | (t, J = 8.0 Hz, 1H), 7.10 (dd, J = 7.6, 2.0 Hz, 1H), 5.94 (s, 1H), 5.07- |
| | | | | 4.78 (m, 1H), 4.66-4.45 (m, 1H), 4.18 (t, J = 8.4 Hz, 2H), 4.01-3.83 |
| | | | | (m, 1H), 3.82-3.54 (m, 7H), 3.48-3.28 (m, 2H), 3.28-3.15 (m, 4H), |
| | | | | 2.96 (d, J = 4.8 Hz, 3H), 2.82-2.68 (m, 2H), 2.42 (s, 3H), 2.26-2.11 |
| | | | | (m, 2H), 2.09-1.97 (m, 2H), 1.91-1.75 (m, 1H), 1.49-1.34 (m, 1H) |
| I-1021 | OA | MM | 896.3 | 10.49 (s, 1H), 10.21 (s, 1H), 9.15 (s, 1H), 8.72-8.62 (m, 2H), 8.50 (s, |
| | | | | 1H), 8.36 (d, J = 1.5 Hz, 1H), 8.23 (d, J = 8.1 Hz, 1H), 7.98-7.87 (m, |
| | | | | 2H), 7.65 (d, J = 8.2 Hz, 1H), 7.58 (d, J = 2.1 Hz, 1H), 7.41 (dd, J = 8.3, |
| | | | | 2.1 Hz, 1H), 4.33-4.23 (m, 1H), 4.16-4.00 (m, 3H), 3.98 (s, 3H), 3.82- |
| | | | | 3.69 (m, 3H), 3.67-3.56 (m, 1H), 3.55-3.36 (m, 3H), 3.22-3.01 (m, 5H), |
| | | | | 2.93-2.83 (m, 1H), 2.80-2.70 (m, 4H), 2.61-2.55 (m, 2H), 2.12 (s, 3H), |
| | | | | 1.89-1.77 (m, 2H), 1.77-1.54 (m, 4H) |
| I-1000 | MR | MS | 942.5 | 10.44 (s, 1H), 9.02 (d, J = 8.8 Hz, 1H), 7.91 (s, 1H), 7.75-7.57 (m, |
| | | | | 2H), 7.43 (d, J = 1.6 Hz, 1H), 7.36 (d, J = 1.6 Hz, 1H), 7.17 (t, J = 7.6 |
| | | | | Hz, 1H), 6.87 (d, J = 7.6 Hz, 1H), 5.94 (s, 1H), 4.43-4.27 (m, 1H), 4.19 |
| | | | | (t, J = 8.4 Hz, 2H), 3.90-3.70 (m, 2H), 3.64-3.46 (m, 5H), 3.30-3.26 |
| | | | | (m, 3H), 3.26-3.13 (m, 5H), 2.96 (d, J = 4.8 Hz, 3H), 2.93-2.76 (m, |
| | | | | 3H), 2.76-2.59 (m, 5H), 2.41-2.20 (m, 4H), 2.20-2.01 (m, 3H), 1.89- |
| | | | | 1.65 (m, 2H), 1.62-1.30 (m, 10H) |
| I-993 | MH | OB | 906.4 | 10.37 (s, 1H), 9.06-8.99 (m, 1H), 8.48 (s, 1H), 7.97-7.87 (m, 2H), 7.79- |
| | | | | 7.66 (m, 2H), 7.35-7.30 (m, 1H), 7.20-7.10 (m, 3H), 6.00-5.93 (m, 1H), |
| | | | | 4.40-4.30 (m, 1H), 4.19 (t, J = 8.6 Hz, 2H), 3.95-3.71 (m, 6H), 3.55- |
| | | | | 3.49 (m, 1H), 3.34-3.33 (m, 4H), 3.25-3.19 (m, 7H), 2.96 (d, J = 4.9 Hz, |
| | | | | 3H), 2.86-2.64 (m, 2H), 2.31 (s, 3H), 2.14-2.02 (m, 7H), 1.60-1.37 (m, 2H) |
| I-981 | OJ | MM | 908.3 | 10.52 (s, 1H), 9.08-9.03 (m, 1H), 8.66-8.62 (m, 1H), 7.94-7.82 (m, 3H), |
| | | | | 7.75-7.67 (m, 3H), 7.59-7.53 (m, 1H), 7.42-7.37 (m, 1H), 7.35-7.30 (m, |
| | | | | 2H), 6.01-5.95 (m, 1H), 4.41-4.30 (m, 1H), 4.20 (t, J = 8.7 Hz, 2H), |
| | | | | 3.83-3.72 (m, 3H), 3.68-3.60 (m, 2H), 3.56-3.44 (m, 4H), 3.33-3.32 (m, |
| | | | | 1H), 3.23-3.11 (m, 5H), 3.03-2.95 (m, 4H), 2.79-2.71 (m, 2H), 2.15-1.99 |
| | | | | (m, 4H), 1.60-1.38 (m, 2H), 1.27-1.19 (m, 4H) |
| I-979 | OO | MM | 908.3 | 10.52 (s, 1H), 9.05 (d, J = 8.8 Hz, 1H), 8.66-8.61 (m, 1H), 7.92 (s, 1H), |
| | | | | 7.90-7.86 (m, 1H), 7.85-7.81 (m, 1H), 7.76-7.72 (m, 1H), 7.71-7.67 (m, |
| | | | | 2H), 7.60-7.54 (m, 1H), 7.41-7.35 (m, 1H), 7.35-7.30 (m, 2H), 5.97 (s, |
| | | | | 1H), 4.41-4.31 (m, 1H), 4.20 (t, J = 8.5 Hz, 2H), 4.01-3.89 (m, 1H), |
| | | | | 3.83-3.73 (m, 2H), 3.69-3.59 (m, 2H), 3.55-3.44 (m, 3H), 3.43-3.27 (m, |
| | | | | 3H), 3.24-3.12 (m, 5H), 3.02-2.94 (m, 4H), 2.78-2.72 (m, 2H), 2.16- |
| | | | | 2.00 (m, 4H), 1.59-1.37 (m, 2H), 1.21 (d, J = 6.4 Hz, 3H) |
| I-976 | MO | OP | 929.4 | 10.69 (s, 1H), 9.03 (d, J = 8.7 Hz, 1H), 8.73 (s, 1H), 7.91 (s, 1H), 7.77- |
| | | | | 7.63 (m, 3H), 7.17 (t, J = 7.8 Hz, 1H), 6.87 (d, J = 7.6 Hz, 1H), 5.94 |
| | | | | (s, 1H), 4.34 (p, J = 8.6 Hz, 1H), 4.20 (t, J = 8.5 Hz, 2H), 3.83-3.74 (m, |
| | | | | 3H), 3.644-3.58 (m, 2H), 3.57-3.46 (m, 2H), 3.24-3.17 (m, 5H), 2.99- |
| | | | | 2.85 (m, 5H), 2.77-2.65 (m, 8H), 2.35-2.24 (m, 1H), 2.20-1.99 (m, |
| | | | | 3H), 1.83-1.72 (m, 2H), 1.61-137 (m, 11H) |
| I-972 | OS | ME | 940.3 | 10.49 (s, 1H), 9.02 (d, J = 8.8 Hz, 1H), 8.49 (s, 1H), 7.95-7.87 (m, |
| | | | | 2H), 7.81-7.64 (m, 2H), 7.45-7.38 (m, 2H), 7.32 (t, J = 8.0 Hz, 1H), |
| | | | | 7.12 (dd, J = 7.2, 1.6 Hz, 1H), 5.97 (d, J = 2.4 Hz, 1H), 4.42-4.29 (m, |
| | | | | 1H), 4.20 (t, J = 8.4 Hz, 2H), 3.97-3.81 (m, 2H), 3.81-3.58 (m, 6H), |
| | | | | 3.54-3.36 (m, 5H), 3.24-3.19 (m, 4H), 2.97 (d, J = 4.8 Hz, 3H), 2.81- |
| | | | | 2.70 (m, 2H), 2.42 (s, 3H), 2.18-1.99 (m, 4H), 1.62-1.39 (m, 2H), |
| | | | | 1.06 (t, J = 7.2 Hz, 3H) |
| I-968 | MD | OP | 887.3 | 10.70 (s, 1H), 8.87 (d, J = 4.8 Hz, 1H), 8.78 (s, 1H), 8.54-8.44 (m, |
| | | | | 1H), 7.97 (s, 1H), 7.89-7.65 (m, 4H), 7.30 (t, J = 8.0 Hz, 1H), 7.10 (dd, |
| | | | | J = 7.6, 2.0 Hz, 1H), 6.01 (s, 1H), 5.01-4.75 (m, 1H), 4.14 (t, J = 8.4 |
| | | | | Hz, 2H), 4.03-3.88 (m, 2H), 3.87-3.66 (m, 5H), 3.60-3.47 (m, 1H), |
| | | | | 3.42-3.35 (m, 2H), 3.27-3.12 (m, 4H), 3.10-3.01 (m, 1H), 2.97 (d, |
| | | | | J = 4.8 Hz, 3H), 2.75 (t, J = 6.4 Hz, 2H), 2.16-2.01 (m, 2H), 1.30- |
| | | | | 1.14 (m, 1H), 1.07-0.89 (m, 1H) |
| I-962 | OU | MM | 946.4 | 10.51 (s, 1H), 8.85 (d, J = 8.4 Hz, 1H), 7.93 (s, 1H), 7.75-7.67 (m, |
| | | | | 1H), 7.67-7.51 (m, 3H), 7.39 (dd, J = 8.0, 2.0 Hz, 1H), 6.66 (dd, J = |
| | | | | 10.0, 2.4 Hz, 1H), 5.91 (d, J = 2.8 Hz, 1H), 4.39-4.27 (m, 1H), 4.23 (t, |
| | | | | J = 8.4 Hz, 2H), 3.84-3.71 (m, 2H), 3.69-3.44 (m, 5H), 3.30-3.24 |
| | | | | (m, 2H), 3.22-3.09 (m, 5H), 3.02-2.85 (m, 5H), 2.85-2.58 (m, 7H), |
| | | | | 2.42-2.25 (m, 1H), 2.23-1.94 (m, 3H), 1.90-1.67 (m, 2H), 1.59- |
| | | | | 1.33 (m, 10H) |
| I-959 | OV | MM | 946.3 | 10.51 (s, 1H), 8.84 (d, J = 8.8 Hz, 1H), 7.93 (s, 1H), 7.75-7.50 (m, |
| | | | | 4H), 7.38 (dd, J = 8.0, 2.0 Hz, 1H), 6.65 (d, J = 9.6 Hz, 1H), 5.96-5.83 |
| | | | | (m, 1H), 4.39-4.27 (m, 1H), 4.26-4.15 (m, 2H), 3.84-3.70 (m, 2H), |
| | | | | 3.70-3.44 (m, 5H), 3.30-3.25 (m, 1H), 3.22-3.08 (m, 5H), 3.02- |
| | | | | 2.83 (m, 5H), 2.83-2.57 (m, 7H), 2.39-2.25 (m, 1H), 2.22-1.94 (m, |
| | | | | 3H), 1.89-1.68 (m, 2H), 1.60-1.31 (m, 11H) |
| I-952 | PA | MM | 926.4 | 10.52 (s, 1H), 9.03 (d, J = 8.7 Hz, 1H), 8.53 (t, J = 1.7 Hz, 1H), 7.94- |
| | | | | 7.88 (m, 2H), 7.77 (dd, J = 11.3, 1.6 Hz, 1H), 7.72-7.66 (m, 2H), 7.57 |
| | | | | (s, 1H), 7.39 (dd, J = 8.2, 2.0 Hz, 1H), 7.33 (t, J = 7.9 Hz, 1H), 7.16- |
| | | | | 7.09 (m, 1H), 5.98 (s, 1H), 4.35 (p, J = 8.5 Hz, 1H), 4.20 (t, J = 8.5 Hz, |
| | | | | 2H), 4.03-3.88 (m, 3H), 3.82-3.73 (m, 2H), 3.68-3.57 (m, 3H), 3.48- |
| | | | | 3.27 (m, 2H), 3.25-3.13 (m, 7H), 3.07-3.03 (m, 1H), 2.97 (d, J = 4.9 Hz, |
| | | | | 3H), 2.79-2.72 (m, 2H), 2.17-1.98 (m, 4H), 1.60-1.37 (m, 2H), 1.23 |
| | | | | (d, J = 6.3 Hz, 3H) |
| I-951 | ML | OP | 947.3 | 10.67 (s, 1H), 8.85 (d, J = 8.4 Hz, 1H), 8.73 (s, 1H), 7.93 (s, 1H), 7.74 |
| | | | | (s, 1H), 7.72-7.66 (m, 1H), 7.63-7.55 (m, 1H), 6.72-6.56 (m, 1H), |
| | | | | 5.92 (s, 1H), 4.40-4.27 (m, 1H), 4.23 (t, J = 8.4 Hz, 2H), 3.86-3.72 |
| | | | | (m, 3H), 3.68-3.57 (m, 2H), 3.57-3.45 (m, 2H), 3.22-3.10 (m, 5H), |
| | | | | 3.01-2.78 (m, 6H), 2.78-2.62 (m, 7H), 2.41-2.24 (m, 1H), 2.23- |
| | | | | 1.95 (m, 3H), 1.90-1.70 (m, 2H), 1.58-1.34 (m, 11H) |
| I-946 | PC | ME | 918.3 | 10.51 (s, 1H), 8.74 (d, J = 4.5 Hz, 1H), 8.50 (s, 1H), 7.99 (s, 1H), 7.80- |
| | | | | 7.71 (m, 3H), 7.40 (s, 2H), 6.90-6.86 (m, 1H), 5.97 (s, 1H), 4.96-4.73 |
| | | | | (m, 1H), 4.19 (t, J = 8.5 Hz, 2H), 3.94-3.85 (m, 1H), 3.78-3.61 (m, |
| | | | | 6H), 3.43-3.34 (m, 3H), 3.25-3.15 (m, 4H), 3.08-2.95 (m, 4H), 2.84- |
| | | | | 2.68 (m, 2H), 2.42 (s, 3H), 2.10-2.00 (m, 2H), 1.25-1.15 (m, 1H), 1.03- |
| | | | | 0.92 (m, 1H) |
| I-944 | PC | MS | 918.3 | 10.49 (s, 1H), 8.74 (d, J = 4.5 Hz, 1H), 8.49 (s, 1H), 7.99 (s, 1H), 7.80- |
| | | | | 7.72 (m, 3H), 7.44 (d, J = 1.6 Hz, 1H), 7.39 (s, 1H), 6.88 (dt, J = 9.8, |
| | | | | 2.1 Hz, 1H), 5.97 (s, 1H), 4.94-4.74 (m, 1H), 4.19 (t, J = 8.6 Hz, 2H), |
| | | | | 3.93-3.80 (m, 3H), 3.75 (s, 2H), 3.770-3.65 (m, 1H), 3.61-3.56 (m, 1H), |
| | | | | 3.45-3.34 (m, 3H), 3.25-3.11 (m, 4H), 3.06-2.93 (m, 4H), 2.87-2.80 |
| | | | | (m, 1H), 2.76-2.65 (m, 1H), 2.25 (s, 3H), 2.09-2.01 (mm 2H), 1.21- |
| | | | | 1.15 (mm, 1H), 1.04-0.94 (m, 1H) |
| I-943 | PG | MM | 896.3 | 10.52 (s, 1H), 10.24 (s, 1H), 9.16 (s, 1H), 9.05 (d, J = 2.3 Hz, 1H), 8.65 |
| | | | | (s, 1H), 8.52 (s, 1H), 8.31 (dd, J = 8.0, 2.4 Hz, 1H), 8.13-8.08 (m, 1H), |
| | | | | 7.65 (d, J = 8.2 Hz, 1H), 7.63-7.56 (m, 3H), 7.41 (dd, J = 8.2, 2.0 Hz, |
| | | | | 1H), 4.30 (d, J = 9.7 Hz, 1H), 4.25-4.15 (m, 1H), 4.13-3.99 (m, 3H), |
| | | | | 3.97 (s, 3H), 3.83 (s, 2H), 3.81-3.72 (m, 1H), 3.68-3.58 (m, 1H), 3.53- |
| | | | | 3.42 (m, 1H), 3.18 (s, 3H), 3.14-3.04 (m, 1H), 2.91-2.71 (m, 5H), 2.65- |
| | | | | 2.53 (m, 4H), 2.12 (s, 3H), 1.92-1.80 (m, 2H), 1.78-1.57 (m, 4H) |
| I-942 | PI | ME | 889.3 | 10.48 (s, 1H), 9.03 (d, J = 8.8 Hz, 1H), 8.43-8.38 (m, 1H), 7.95-7.86 (m, |
| | | | | 2H), 7.74-7.64 (m, 2H), 7.40-7.36 (m, 2H), 7.32 (t, J = 7.9 Hz, 1H), |
| | | | | 7.15-7.08 (m, 1H), 5.97 (s, 1H), 4.41-4.29 (m, 1H), 4.19 (t, J = 8.5 Hz, |
| | | | | 2H), 3.81-3.67 (m, 2H), 3.65-3.46 (m, 4H), 3.24-3.17 (m, 5H), 2.97 (d, |
| | | | | J = 4.9 Hz, 3H), 2.86-2.80 (m, 2H), 2.80-2.70 (m, 2H), 2.67-2.56 (m, |
| | | | | 2H), 2.40 (s, 3H), 2.15-1.90 (m, 4H), 1.69-1.39 (m, 8H) |
| I-939 | PJ | MM | 926.4 | 10.52 (s, 1H), 9.03 (d, J = 8.8 Hz, 1H), 8.53 (d, J = 1.6 Hz, 1H), 7.98- |
| | | | | 7.87 (m, 2H), 7.77 (dd, J = 11.2, 1.6 Hz, 1H), 7.72-7.65 (m, 2H), 7.62- |
| | | | | 7.53 (m, 1H), 7.39 (dd, J = 8.0, 2.0 Hz, 1H), 7.32 (t, J = 8.0 Hz, 1H), |
| | | | | 7.12 (dd, J = 7.6, 2.0 Hz, 1H), 5.97 (d, J = 2.4 Hz, 1H), 4.43-4.29 (m, |
| | | | | 1H), 4.19 (t, J = 8.4 Hz, 2H), 4.04-3.91 (m, 1H), 3.88-3.71 (m, 3H), |
| | | | | 3.71-3.51 (m, 4H), 3.47-3.37 (m, 1H), 3.27-3.13 (m, 7H), 3.11- |
| | | | | 3.02 (m, 1H), 2.97 (d, J = 4.8 Hz, 3H), 2.85-2.67 (m, 2H), 2.21-1.95 |
| | | | | (m, 4H), 1.61-1.36 (m, 2H), 1.23 (d, J = 6.4 Hz, 3H) |
| I-933 | MH | PK | 927.3 | 10.64 (s, 1H), 9.08-8.99 (m, 1H), 8.52-8.47 (m, 1H), 7.94-7.89 (m, 2H), |
| | | | | 7.79-7.74 (m, 1H), 7.72-7.65 (m, 2H), 7.35-7.30 (m, 1H), 7.14-7.11 (m, |
| | | | | 1H), 5.97 (s, 1H), 4.41-4.30 (m, 1H), 4.20 (t, J = 8.5 Hz, 2H), 3.99-3.87 |
| | | | | (m, 2H), 3.82-3.66 (m, 6H), 3.56-3.48 (m, 1H), 3.39-3.34 (m, 2H), 3.25- |
| | | | | 3.20 (m, 7H), 2.97 (d, J = 4.9 Hz, 3H), 2.75 (t, J = 6.6 Hz, 2H), 2.64 (s, |
| | | | | 3H), 2.16-2.01 (m, 4H), 1.59-1.39 (m, 2H) |
| I-930 | MD | PK | 901.2 | 10.65 (s, 1H), 8.89-8.85 (m, 1H), 8.51-8.48 (m, 1H), 7.97 (s, 1H), 7.81- |
| | | | | 7.68 (m, 3H), 7.68-7.65 (m, 1H), 7.32-7.27 (m, 1H), 7.12-7.09 (m, 1H), |
| | | | | 6.01 (s, 1H), 4.99-4.77 (m, 1H), 4.15 (t, J = 8.5 Hz, 2H), 4.00-3.86 (m, |
| | | | | 2H), 3.80-3.68 (m, 4H), 3.54-3.49 (m, 1H), 3.40-3.33 (m, 3H), 3.25- |
| | | | | 3.15 (m, 4H), 3.09-3.01 (m, 1H), 2.97 (d, J = 4.9 Hz, 3H), 2.75 (t, J = |
| | | | | 6.6 Hz, 2H), 2.64 (s, 3H), 2.11-2.04 (m, 2H), 1.30-1.17 (m, 1H), 1.05- |
| | | | | 0.92 (m, 1H) |
| I-925 | MH | OP | 913.3 | 10.70 (s, 1H), 9.06-9.01 (m, 1H), 8.78 (s, 1H), 8.49 (s, 1H), 7.94-7.88 |
| | | | | (m, 2H), 7.86-7.81 (m, 1H), 7.79-7.68 (m, 2H), 7.36-7.30 (m, 1H), 7.15- |
| | | | | 7.10 (m, 1H), 5.99-5.94 (m, 1H), 4.40-4.31 (m, 1H), 4.19 (t, J = 8.0 Hz, |
| | | | | 2H), 4.01-3.88 (m, 3H), 3.84-3.70 (m, 6H), 3.55-3.48 (m, 1H), 3.38- |
| | | | | 3.36 (m, 1H), 3.24-3.20 (m, 7H), 2.96 (d, J = 4.9 Hz, 3H), 2.75 (t, J = |
| | | | | 6.6 Hz, 2H), 2.14-2.02 (m, 4H), 1.56-1.41 (m, 2H) |
| I-1156 | NC | PK | 915.3 | 10.61 (s, 1H), 9.03 (d, J = 8.7 Hz, 1H), 7.93-7.89 (m, 2H), 7.70 (d, J = |
| | | | | 8.0 Hz, 1H), 7.68-7.62 (m, 1H), 7.22-7.15 (m, 1H), 6.88 (d, J = 7.6 Hz, |
| | | | | 1H), 5.95 (s, 1H), 4.39-4.32 (m, 1H), 4.27 (s, 2H), 4.20 (t, J = 8.6 Hz, |
| | | | | 2H), 3.88-3.73 (m, 5H), 3.59-3.47 (m, 2H), 3.26-3.16 (m, 5H), 2.99- |
| | | | | 2.81 (m, 6H), 2.79-2.73 (m, 3H), 2.70-2.61 (m, 6H), 2.36-2.24 (m, 1H), |
| | | | | 2.21-2.01 (m, 3H), 1.86-1.64 (m, 6H), 1.60-1.37 (m, 2H) |
| I-1150 | PC | PK | 919.4 | 10.66 (s, 1H), 8.73 (d, J = 4.5 Hz, 1H), 8.49 (d, J = 1.8 Hz, 1H), 7.98 (s, |
| | | | | 1H), 7.81-7.64 (m, 4H), 6.92-6.84 (m, 1H), 5.96 (s, 1H), 4.98-4.68 |
| | | | | (m, 1H), 4.18 (t, J = 8.4 Hz, 2H), 4.00-3.87 (m, 2H), 3.79-3.68 (m, |
| | | | | 4H), 3.54-3.48 (m, 2H), 3.38-3.30 (m, 2H), 3.25-3.13 (m, 4H), 3.06- |
| | | | | 2.99 (m, 1H), 2.96 (d, J = 4.9 Hz, 3H), 2.75 (t, J = 6.6 Hz, 2H), 2.63 |
| | | | | (s, 3H), 2.11-2.02 (m, 2H), 1.28-1.11 (m, 1H), 1.07-0.89 (m, 1H) |
| I-1151 | MZ | PK | 945.4 | 10.66 (s, 1H), 8.85 (d, J = 8.7 Hz, 1H), 8.49 (d, J = 2.1 Hz, 1H), 7.95 (s, |
| | | | | 1H), 7.88-7.64 (m, 4H), 6.95-6.85 (m, 1H), 5.93 (s, 1H), 4.39-4.16 |
| | | | | (m, 3H), 4.01-3.65 (m, 8H), 3.54-3.50 (m, 1H), 3.39-3.36 (m, 1H), |
| | | | | 3.31-3.27 (m, 1H), 3.26-3.13 (m, 7H), 2.96 (d, J = 4.9 Hz, 3H), 2.75 |
| | | | | (t, J = 6.6 Hz, 2H), 2.63 (s, 3H), 2.14-1.95 (m, 4H), 1.56-1.41 (m, 2H) |
| I-1170 | XQ | PK | 915.4 | 10.63 (s, 1H), 9.03 (d, J = 8.8 Hz, 1H), 7.97-7.88 (m, 2H), 7.76-7.62 |
| | | | | (m, 2H), 7.17 (t, J = 7.6 Hz, 1H), 6.88 (d, J = 7.6 Hz, 1H), 5.95 (s, 1H), |
| | | | | 4.41-4.25 (m, 3H), 4.20 (t, J = 8.4 Hz, 2H), 3.90-3.63 (m, 5H), 3.61- |
| | | | | 3.46 (m, 2H), 3.26-3.15 (m, 5H), 3.01-2.82 (m, 6H), 2.75 (t, J = 6.4 |
| | | | | Hz, 2H), 2.71-2.58 (m, 7H), 2.39-2.22 (m, 1H), 2.21-1.99 (m, 3H), |
| | | | | 1.87-1.63 (m, 6H), 1.61-1.36 (m, 2H) |
| I-1169 | XS | PK | 915.4 | 10.63 (s, 1H), 9.02 (d, J = 8.8 Hz, 1H), 7.98-7.85 (m, 2H), 7.77-7.58 |
| | | | | (m, 2H), 7.17 (t, J = 7.6 Hz, 1H), 6.87 (d, J = 7.6 Hz, 1H), 5.94 (s, 1H), |
| | | | | 4.44-4.09 (m, 5H), 3.88-3.44 (m, 7H), 3.28-3.12 (m, 5H), 3.01- |
| | | | | 2.85 (m, 5H), 2.81-2.58 (m, 10H), 2.41-2.22 (m, 1H), 2.22-1.98 |
| | | | | (m, 3H), 1.89-1.60 (m, 6H), 1.60-1.35 (m, 2H) |
| I-1175 | XM | PK | 919.2 | 10.65 (s, 1H), 8.99-8.95 (m, 1H), 8.49-8.45 (m, 1H), 7.99-7.95 (m, 1H), |
| | | | | 7.87-7.63 (m, 4H), 7.35-7.24 (m, 1H), 7.18-7.05 (m, 1H), 6.07-5.93 (m, |
| | | | | 1H), 4.21-4.09 (m, 2H), 4.05-3.86 (m, 3H), 3.85-3.59 (m, 6H), 3.56- |
| | | | | 3.48 (m, 1H), 3.27-3.14 (m, 4H), 3.02-2.90 (m, 3H), 2.80-2.71 (m, 2H), |
| | | | | 2.72-2.55 (m, 4H), 2.15-1.99 (m, 3H), 1.70-1.54 (m, 1H) |
| I-1174 | NT | PK | 915.3 | 10.65 (s, 1H), 9.14-8.95 (m, 1H), 7.91 (s, 1H), 7.78-7.55 (m, 3H), 7.27- |
| | | | | 7.08 (m, 1H), 6.95-6.81 (m, 1H), 5.94 (s, 1H), 4.41-4.29 (m, 1H), 4.23- |
| | | | | 4.09 (m, 2H), 3.89-3.72 (m, 3H), 3.66-3.48 (m, 5H), 3.25-3.14 (m, 6H), |
| | | | | 3.12-3.01 (m, 4H), 2.99-2.90 (m, 3H), 2.85-2.70 (m, 4H), 2.67-2.59 (m, |
| | | | | 4H), 2.43-2.20 (m, 2H), 2.15-1.98 (m, 2H), 1.82-1.60 (m, 5H), 1.57- |
| | | | | 1.36 (m, 3H) |
| I-1152 | PC | OP | 905.2 | 10.69 (s, 1H), 8.78 (s, 1H), 8.73 (d, J = 4.5 Hz, 1H), 8.49 (t, J = 1.7 Hz, |
| | | | | 1H), 7.98 (s, 1H), 7.86-7.68 (m, 4H), 6.92-6.84 (m, 1H), 5.97 (s, 1H), |
| | | | | 4.95-4.73 (m, 1H), 4.19 (t, J = 8.5 Hz, 2H), 4.01-3.86 (m, 2H), 3.85- |
| | | | | 3.68 (m, 5H), 3.56-3.48 (m, 1H), 3.41-3.36 (m, 2H), 3.25-3.19 |
| | | | | (m, 2H), 3.16 (t, J = 8.5 Hz, 2H), 3.06-2.99 (m, 1H), 2.97 (d, J = 4.9 |
| | | | | Hz, 3H), 2.75 (t, J = 6.6 Hz, 2H), 2.11-2.01 (m, 2H), 1.25-1.14 (m, |
| | | | | 1H), 1.04-0.91 (m, 1H) |
| I-1180 | OV | VX | 961.4 | 10.64 (s, 1H), 8.84 (d, J = 8.4 Hz, 1H), 7.92 (s, 1H), 7.69 (d, J = 5.2 Hz, |
| | | | | 1H), 7.62-7.54 (m, 2H), 6.72-6.59 (m, 1H), 5.90 (s, 1H), 4.39-4.26 |
| | | | | (m, 1H), 4.22 (t, J = 8.4 Hz, 2H), 3.87-3.71 (m, 2H), 3.66-3.55 (m, |
| | | | | 2H), 3.56-3.42 (m, 2H), 3.40-3.27 (m, 3H), 3.25-3.09 (m, 5H), 3.02- |
| | | | | 2.85 (m, 5H), 2.82-2.64 (m, 7H), 2.61 (s, 3H), 2.41-2.23 (m, 1H), |
| | | | | 2.23-2.12 (m, 1H), 2.12-1.95 (m, 2H), 1.90-1.67 (m, 2H), 1.61- |
| | | | | 1.32 (m, 10H) |
| I-1179 | WA | VX | 915.5 | 10.64 (s, 1H), 9.08 (d, J = 8.8 Hz, 1H), 7.90 (s, 1H), 7.76-7.57 (m, 2H), |
| | | | | 7.41 (d, J = 8.0 Hz, 1H), 7.12 (t, J = 8.0 Hz, 1H), 6.59 (d, J = 8.0 Hz, |
| | | | | 1H), 5.92 (d, J = 3.6 Hz, 1H), 4.41-4.28 (m, 1H), 4.15 (t, J = 8.0 Hz, |
| | | | | 2H), 4.01-3.60 (m, 6H), 3.55-3.45 (m, 1H), 3.29-3.01 (m, 11H), |
| | | | | 2.95 (d, J = 5.2 Hz, 3H), 2.75 (t, J = 6.4 Hz, 2H), 2.70-2.54 (m, 5H), |
| | | | | 2.41-2.28 (m, 2H), 2.17-1.92 (m, 4H), 1.86-1.70 (m, 2H), 1.59- |
| | | | | 1.34 (m, 3H), 1.35-1.16 (m, 2H) |
| I-1177 | WC | VX | 898.4 | 10.66 (s, 1H), 9.01 (d, J = 8.8 Hz, 1H), 7.91 (s, 1H), 7.76-7.59 (m, 3H), |
| | | | | 7.13 (t, J = 7.6 Hz, 1H), 6.71 (d, J = 7.6 Hz, 1H), 5.93 (s, 1H), 4.45- |
| | | | | 4.28 (m, 1H), 4.19 (t, J = 8.4 Hz, 2H), 4.03-3.61 (m, 6H), 3.51 (s, 1H), |
| | | | | 3.31-3.11 (m, 9H), 2.95 (d, J = 4.8 Hz, 3H), 2.75 (t, J = 6.8 Hz, 2H), |
| | | | | 2.70-2.54 (m, 5H), 2.15-1.93 (m, 10H), 1.61-1.35 (m, 2H) |
| I-1178 | WD | VX | 941.4 | 10.65 (s, 1H), 9.01 (d, J = 8.8 Hz, 1H), 7.91 (s, 1H), 7.71-7.61 (m, 3H), |
| | | | | 7.13 (t, J = 7.8 Hz, 1H), 6.72 (d, J = 7.5 Hz, 1H), 5.93 (s, 1H), 4.71-4.50 |
| | | | | (m, 1H), 4.39-4.30 (m, 1H), 4.19 (t, J = 8.7 Hz, 2H), 3.94-3.55 (m, 4H), |
| | | | | 3.29-3.22 (m, 3H), 3.22-3.16 (m, 4H), 3.14-3.01 (m, 1H), 2.96 (d, J = |
| | | | | 4.8 Hz, 3H), 2.79-2.70 (m, 6H), 2.67-2.60 (m, 4H), 2.46-2.40 (m, 5H), |
| | | | | 2.13-2.01 (m, 8H), 1.94-1.74 (m, 2H), 1.59-1.36 (m, 2H) |
| I-1181 | MR | VX | 943.5 | 10.62 (s, 1H), 9.02 (d, J = 8.8 Hz, 1H), 7.91 (s, 1H), 7.76-7.62 (m, 2H), |
| | | | | 7.58 (s, 1H), 7.18 (t, J = 7.6 Hz, 1H), 6.88 (d, J = 7.6 Hz, 1H), 5.96 (s, |
| | | | | 1H), 4.44-4.28 (m, 1H), 4.20 (t, J = 8.4 Hz, 2H), 3.86-3.69 (m, 3H), |
| | | | | 3.66-3.47 (m, 4H), 3.41-3.33 (m, 2H), 3.27-3.15 (m, 5H), 3.04- |
| | | | | 2.83 (m, 5H), 2.80-2.65 (m, 6H), 2.61 (s, 3H), 2.42-2.23 (m, 1H), |
| | | | | 2.22-1.97 (m, 4H), 1.88-1.67 (m, 2H), 1.61-1.33 (m, 10H) |
| I-1183 | WF | ME | 956.4 | 10.48 (s, 1H), 9.03 (d, J = 8.8 Hz, 1H), 7.91 (s, 1H), 7.76-7.64 (m, 2H), |
| | | | | 7.43-7.34 (m, 2H), 7.17 (t, J = 7.6 Hz, 1H), 6.87 (d, J = 7.6 Hz, 1H), |
| | | | | 5.95 (s, 1H), 4.50-4.28 (m, 2H), 4.20 (t, J = 8.4 Hz, 2H), 3.83-3.68 |
| | | | | (m, 2H), 3.67-3.51 (m, 4H), 3.27-3.15 (m, 5H), 3.09-2.89 (m, 4H), |
| | | | | 2.83-2.58 (m, 7H), 2.49-2.44 (m, 2H), 2.41 (s, 3H), 2.20-1.95 (m, |
| | | | | 6H), 1.87-1.63 (m, 7H), 1.63-1.37 (m, 4H), 1.16-0.97 (m, 2H) |
| I-1184 | WF | PK | 957.4 | 10.65 (s, 1H), 9.03 (d, J = 8.8 Hz, 1H), 8.16 (s, 1H) (FA), 7.91 (s, 1H), |
| | | | | 7.76-7.65 (m, 2H), 7.57 (s, 1H), 7.17 (t, J = 7.6 Hz, 1H), 6.87 (d, J = |
| | | | | 7.6 Hz, 1H), 5.95 (s, 1H), 4.51-4.41 (m, 1H), 4.41-4.27 (m, 1H), 4.20 |
| | | | | (t, J = 8.4 Hz, 2H), 3.83-3.73 (m, 2H), 3.71-3.62 (m, 2H), 3.59-3.51 |
| | | | | (m, 2H), 3.27-3.14 (m, 5H), 3.10-2.93 (m, 4H), 2.84-2.71 (m, 3H), |
| | | | | 2.71-2.59 (m, 7H), 2.49-2.42 (m, 2H), 2.24-1.98 (m, 6H), 1.87- |
| | | | | 1.76 (m, 2H), 1.76-1.38 (m, 9H), 1.16-0.98 (m, 2H) |
| I-1190 | MR | OP | 929.4 | 10.63 (s, 1H), 9.02 (d, J = 8.7 Hz, 1H), 8.73 (s, 1H), 7.91 (s, 1H), 7.75- |
| | | | | 7.62 (m, 3H), 7.17 (t, J = 7.8 Hz, 1H), 6.87 (d, J = 7.5 Hz, 1H), 5.95 (s, |
| | | | | 1H), 4.41-4.28 (m, 1H), 4.20 (t, J = 8.5 Hz, 2H), 3.84-3.73 (m, 3H), |
| | | | | 3.66-3.57 (m, 2H), 3.56-3.48 (m, 2H), 3.38-3.32 (m, 2H), 3.26-3.17 (m, |
| | | | | 5H), 3.00-2.85 (m, 5H), 2.80-2.58 (m, 7H), 2.39-2.21 (m, 1H), 2.20- |
| | | | | 2.00 (m, 3H), 1.87-1.69 (m, 2H), 1.61-1.36 (m, 10H) |
| I-1187 | WH | VX | 894.4 | 10.64 (s, 1H), 9.03 (d, J = 8.8 Hz, 1H), 7.91 (s, 1H), 7.71-7.61 (m, 2H), |
| | | | | 7.57 (d, J = 4.4 Hz, 1H), 7.15 (t, J = 7.8 Hz, 1H), 6.87 (d, J = 7.6 Hz, |
| | | | | 1H), 5.94 (s, 1H), 4.41-4.28 (m, 1H), 4.19 (t, J = 8.5 Hz, 2H), 4.14-4.00 |
| | | | | (m, 1H), 3.92-3.63 (m, 3H), 3.61-3.55 (m, 1H), 3.55-3.48 (m, 1H), 3.42 |
| | | | | (s, 2H), 3.31-3.26 (m, 2H), 3.25-3.16 (m, 6H), 2.96 (d, J = 4.9 Hz, 3H), |
| | | | | 2.74 (t, J = 6.6 Hz, 2H), 2.71-2.64 (m, 2H), 2.63-2.58 (m, 3H), 2.25-2.15 |
| | | | | (m, 2H), 2.14-2.00 (m, 4H), 1.82-1.74 (m, 2H), 1.70-1.60 (m, 2H), 1.59- |
| | | | | 1.37 (m, 8H) |
| I-1185 | WL | VX | 971.5 | 10.65 (s, 1H), 9.03 (d, J = 8.7 Hz, 1H), 7.91 (s, 1H), 7.73-7.64 (m, 2H), |
| | | | | 7.57 (s, 1H), 7.16 (t, J = 7.8 Hz, 1H), 6.86 (d, J = 7.6 Hz, 1H), 5.94 (s, |
| | | | | 1H), 4.50-4.41 (m, 1H), 4.39-4.29 (m, 1H), 4.19 (t, J = 8.5 Hz, 2H), |
| | | | | 3.82-3.73 (m, 2H), 3.71-3.60 (m, 2H), 3.57-3.46 (m, 2H), 3.24-3.17 (m, |
| | | | | 5H), 3.07-2.85 (m, 8H), 2.82-2.71 (m, 4H), 2.61 (s, 3H), 2.49-2.43 (m, |
| | | | | 2H), 2.36-2.21 (m, 1H), 2.18-2.00 (m, 3H), 1.84-1.69 (m, 3H), 1.66- |
| | | | | 1.52 (m, 5H), 1.50-1.39 (m, 1H), 1.36-1.22 (m, 1H), 1.15-0.96 (m, 6H) |
| I-1186 | WN | VX | 971.5 | 10.67 (s, 1H), 9.03 (d, J = 8.7 Hz, 1H), 7.91 (s, 1H), 7.74-7.65 (m, 2H), |
| | | | | 7.58 (s, 1H), 7.21-7.09 (m, 1H), 6.91-6.82 (m, 1H), 5.96-5.88 (m, 1H), |
| | | | | 4.51-4.38 (m, 1H), 4.38-4.27 (m, 1H), 4.23-4.12 (m, 2H), 3.84-3.60 (m, |
| | | | | 4H), 3.50 (s, 2H), 3.23-3.17 (m, 5H), 3.11-2.99 (m, 1H), 2.98-2.83 (m, |
| | | | | 7H), 2.78-2.66 (m, 4H), 2.61 (s, 3H), 2.48-2.38 (m, 2H), 2.35-2.18 (m, |
| | | | | 1H), 2.16-1.97 (m, 3H), 1.84-1.66 (m, 3H), 1.63-1.37 (m, 6H), 1.35- |
| | | | | 1.21 (m, 1H), 1.15-0.95 (m, 6H) |
| I-1191 | WQ | ME | 925.4 | 10.49 (s, 1H), 9.01 (d, J = 8.8 Hz, 1H), 8.23 (s, 1H), 7.93 (s, 1H), 7.80 |
| | | | | (d, J = 8.0 Hz, 1H), 7.76-7.71 (m, 1H), 7.69 (s, 1H), 7.43-7.38 (m, |
| | | | | 2H), 7.32 (t, J = 8.0 Hz, 1H), 7.22 (d, J = 8.0 Hz, 1H), 5.97 (s, 1H), 4.40- |
| | | | | 4.28 (m, 1H), 4.27-4.11 (m, 3H), 3.82-3.69 (m, 2H), 3.66-3.54 |
| | | | | (m, 3H), 3.50-3.42 (m, 3H), 3.30-3.24 (m, 1H), 3.21 (s, 3H), 3.16- |
| | | | | 3.02 (m, 1H), 2.97 (d, J = 4.8 Hz, 3H), 2.81-2.63 (m, 4H), 2.56-2.52 |
| | | | | (m, 2H), 2.41 (s, 3H), 2.19-1.98 (m, 2H), 1.91-1.75 (m, 2H), 1.73- |
| | | | | 1.60 (m, 3H), 1.60-1.36 (m, 3H) |
| I-1194 | WF | MS | 956.4 | 10.47 (s, 1H), 9.03 (d, J = 8.8 Hz, 1H), 7.90 (s, 1H), 7.73-7.66 (m, 2H), |
| | | | | 7.43 (d, J = 1.6 Hz, 1H), 7.35 (d, J = 1.7 Hz, 1H), 7.17 (t, J = 7.7 Hz, |
| | | | | 1H), 6.87 (d, J = 7.6 Hz, 1H), 5.94 (s, 1H), 4.47-4.39 (m, 1H), 4.38-4.28 |
| | | | | (m, 1H), 4.19 (t, J = 8.5 Hz, 2H), 3.91-3.71 (m, 2H), 3.58-3.50 (m, 3H), |
| | | | | 3.25-3.17 (m, 5H), 3.09-2.99 (m, 1H), 2.95 (d, J = 4.9 Hz, 3H), 2.88- |
| | | | | 2.76 (m, 1H), 2.75-2.58 (m, 5H), 2.47-2.42 (m, 2H), 2.23 (s, 3H), 2.16- |
| | | | | 1.98 (m, 7H), 1.80-1.62 (m, 7H), 1.60-1.35 (m, 5H), 1.09-1.02 (m, 2H) |
| I-1197 | WT | ME | 914.4 | 10.48 (s, 1H), 9.00 (d, J = 8.6 Hz, 1H), 7.91 (s, 1H), 7.80-7.59 (m, 2H), |
| | | | | 7.42-7.37 (m, 2H), 7.27-7.10 (m, 1H), 7.00-6.77 (m, 1H), 5.93 (s, 1H), |
| | | | | 4.52-4.44 (m, 1H), 4.41-4.30 (m, 2H), 4.21 (t, J = 8.8 Hz, 2H), 3.82-3.68 |
| | | | | (m, 3H), 3.66-3.55 (m, 3H), 3.26-3.18 (m, 6H), 3.12-2.90 (m, 6H), 2.80- |
| | | | | 2.58 (m, 7H), 2.41 (s, 3H), 2.16-1.90 (m, 5H), 1.84-1.73 (m, 1H), 1.68- |
| | | | | 1.58 (m, 1H), 1.58-1.49 (m, 1H), 1.48-1.34 (m, 3H) |
| I-1198 | WV | ME | 914.4 | 10.47 (s, 1H), 9.03 (d, J = 8.7 Hz, 1H), 7.91 (s, 1H), 7.74-7.64 (m, 2H), |
| | | | | 7.38 (s, 2H), 7.17 (t, J = 7.8 Hz, 1H), 6.87 (d, J = 7.5 Hz, 1H), 5.95 (s, |
| | | | | 1H), 4.38-4.30 (m, 1H), 4.20 (t, J = 8.5 Hz, 2H), 4.13-4.03 (m, 1H), |
| | | | | 3.80-3.67 (m, 2H), 3.66-3.55 (m, 2H), 3.52 (s, 3H), 3.25-3.12 (m, 7H), |
| | | | | 3.11-2.99 (m, 4H), 2.96 (d, J = 4.9 Hz, 3H), 2.77-2.70 (m, 2H), 2.49- |
| | | | | 2.45 (m, 4H), 2.44-2.26 (m, 3H), 2.14-2.00 (m, 2H), 1.98-1.89 (m, 2H), |
| | | | | 1.77-1.65 (m, 1H), 1.61-1.38 (m, 3H), 1.19-1.08 (m, 2H) |
| I-1188 | XE | ME | 957.3 | 10.50 (s, 1H), 9.08 (d, J = 8.8 Hz, 1H), 7.90 (s, 1H), 7.70-7.62 (m, 1H), |
| | | | | 7.47-7.38 (m, 3H), 7.14 (t, J = 8.0 Hz, 1H), 6.60 (d, J = 8.1 Hz, 1H), |
| | | | | 5.93 (s, 1H), 4.56-4.43 (m, 1H), 4.40-4.27 (m, 1H), 4.16 (t, J = 8.3 Hz, |
| | | | | 2H), 3.81-3.69 (m, 2H), 3.68-3.56 (m, 2H), 3.20 (s, 3H), 3.14-3.02 (m, |
| | | | | 4H), 3.01-2.91 (m, 7H), 2.89-2.82 (m, 1H), 2.80-2.70 (m, 3H), 2.69- |
| | | | | 2.59 (m, 3H), 2.58-2.53 (m, 1H), 2.48-2.39 (m, 6H), 2.38-2.32 (m, 1H), |
| | | | | 2.31-2.24 (m, 1H), 2.14-1.99 (m, 3H), 1.98-1.90 (m, 1H), 1.88-1.76 (m, |
| | | | | 1H), 1.72-1.61 (m, 1H), 1.58-1.28 (m, 5H) |
| I-1189 | XH | ME | 957.4 | 10.49 (s, 1H), 9.08 (d, J = 8.8 Hz, 1H), 7.93-7.88 (m, 1H), 7.70-7.62 (m, |
| | | | | 1H), 7.47-7.38 (m, 3H), 7.14 (t, J = 8.0 Hz, 1H), 6.60 (d, J = 8.1 Hz, |
| | | | | 1H), 5.93 (s, 1H), 4.54-4.42 (m, 1H), 4.40-4.27 (m, 1H), 4.16 (t, J = 8.3 |
| | | | | Hz, 2H), 3.80-3.68 (m, 2H), 3.67-3.56 (m, 2H), 3.20 (s, 3H), 3.14-3.03 |
| | | | | (m, 4H), 3.01-2.92 (m, 7H), 2.90-2.82 (m, 1H), 2.79-2.71 (m, 3H), 2.69- |
| | | | | 2.60 (m, 3H), 2.59-2.53 (m, 1H), 2.47-2.39 (m, 6H), 2.38-2.32 (m, 1H), |
| | | | | 2.31-2.24 (m, 1H), 2.14-2.00 (m, 3H), 1.99-1.91 (m, 1H), 1.88-1.78 (m, |
| | | | | 1H), 1.73-1.61 (m, 1H), 1.56-1.30 (m, 5H) |
| I-1195 | XI | ME | 942.5 | 10.49 (s, 1H), 9.03 (d, J = 8.8 Hz, 1H), 7.91 (s, 1H), 7.73-7.63 (m, 2H), |
| | | | | 7.39 (s, 2H), 7.17 (t, J = 7.8 Hz, 1H), 6.87 (d, J = 7.6 Hz, 1H), 5.95 (s, |
| | | | | 1H), 4.50-4.41 (m, 1H), 4.38-4.30 (m, 1H), 4.20 (t, J = 8.6 Hz, 2H), |
| | | | | 3.82-3.68 (m, 2H), 3.66-3.57 (m, 2H), 3.55 (s, 2H), 3.26-3.14 (m, 5H), |
| | | | | 3.07-3.00 (m, 1H), 2.96 (d, J = 4.9 Hz, 3H), 2.79-2.60 (m, 8H), 2.48- |
| | | | | 2.44 (m, 2H), 2.41 (s, 3H), 2.35-2.24 (m, 2H), 2.14-2.00 (m, 2H), 1.87- |
| | | | | 1.78 (m, 1H), 1.73-1.47 (m, 8H), 1.46-1.34 (m, 3H) |
| I-1196 | WF | OP | 943.4 | 10.68 (s, 1H), 9.03 (d, J = 8.7 Hz, 1H), 8.76-8.70 (m, 1H), 7.93-7.88 (m, |
| | | | | 1H), 7.76-7.72 (m, 1H), 7.72-7.64 (m, 2H), 7.17 (t, J = 7.7 Hz, 1H), 6.87 |
| | | | | (d, J = 7.5 Hz, 1H), 5.95 (s, 1H), 4.50-4.42 (m, 1H), 4.38-4.30 (m, 1H), |
| | | | | 4.20 (t, J = 8.1 Hz, 2H), 3.85-3.72 (m, 3H), 3.70-3.62 (m, 1H), 3.55 (s, |
| | | | | 2H), 3.24-3.12 (m, 5H), 3.09-2.99 (m, 1H), 2.96 (d, J = 4.6 Hz, 3H), |
| | | | | 2.85-2.71 (m, 3H), 2.70-2.60 (m, 4H), 2.48-2.44 (m, 2H), 2.16-1.98 (m, |
| | | | | 6H), 1.84-1.76 (m, 2H), 1.72-1.62 (m, 4H), 1.60-1.37 (m, 5H), 1.11-0.99 |
| | | | | (m, 2H) |
| I-1199 | WF | MN | 942.4 | 10.51 (s, 1H), 9.03 (d, J = 8.8 Hz, 1H), 7.91 (s, 1H), 7.73-7.65 (m, 2H), |
| | | | | 7.65-7.60 (m, 1H), 7.54 (d, J = 2.0 Hz, 1H), 7.38 (dd, J = 8.2, 2.0 Hz, |
| | | | | 1H), 7.17 (t, J = 7.8 Hz, 1H), 6.87 (d, J = 7.6 Hz, 1H), 5.94 (s, 1H), 4.48- |
| | | | | 4.41 (m, 1H), 4.40-4.27 (m, 1H), 4.19 (t, J = 8.5 Hz, 2H), 3.82-3.72 (m, |
| | | | | 2H), 3.68-3.52 (m, 4H), 3.25-3.16 (m, 5H), 3.11-3.01 (m, 1H), 2.96 (d, |
| | | | | J = 4.9 Hz, 3H), 2.79-2.71 (m, 3H), 2.70-2.58 (m, 5H), 2.49-2.43 (m, |
| | | | | 2H), 2.16-1.99 (m, 6H), 1.83-1.63 (m, 6H), 1.60-1.37 (m, 4H), 1.12- |
| | | | | 0.98 (m, 2H) |
| I-1042 | MH | SZ | 893.4 | 10.56 (s, 1H), 9.02 (d, J = 8.8 Hz, 1H), 8.51 (d, J = 16.0 Hz, 2H), 7.96- |
| | | | | 7.87 (m, 2H), 7.79-7.57 (m, 3H), 7.36-7.29 (m, 1H), 7.12 (d, J = 7.6 Hz, |
| | | | | 1H), 6.00-5.93 (m, 1H), 4.40-4.31 (m, 1H), 4.23-4.15 (m, 2H), 4.01- |
| | | | | 3.92 (m, 2H), 3.81-3.70 (m, 6H), 3.58-3.53 (m, 2H), 3.25-3.19 (m, 8H), |
| | | | | 2.97 (d, J = 4.9 Hz, 3H), 2.79-2.72 (m, 2H), 2.24 (s, 3H), 2.14-2.03 (m, |
| | | | | 4H), 1.58-1.41 (m, 2H) |
| I-1012 | MD | SZ | 867.4 | 10.57 (s, 1H), 8.87 (d, J = 4.6 Hz, 1H), 8.55-8.51 (m, 1H), 8.51-8.47 (m, |
| | | | | 1H), 7.97 (s, 1H), 7.81-7.71 (m, 3H), 7.64-7.58 (m, 1H), 7.33-7.26 (m, |
| | | | | 1H), 7.12-7.07 (m, 1H), 6.01 (s, 1H), 5.00-4.78 (m, 1H), 4.15 (m, 2H), |
| | | | | 4.01-3.90 (m, 2H), 3.82-3.68 (m, 4H), 3.58-3.50 (m, 1H), 3.34-3.29 (m, |
| | | | | 2H), 3.25-3.16 (m, 4H), 3.08-3.01 (m, 1H), 2.97 (d, J = 4.9 Hz, 3H), |
| | | | | 2.79-2.72 (m, 2H), 2.55-2.53 (m, 1H), 2.24 (s, 3H), 2.10-2.03 (m, 2H), |
| | | | | 1.28-1.18 (m, 1H), 1.04-0.93 (m, 1H) |
| I-921 | MH | TY | 942.3 | 10.37 (s, 1H), 9.02 (d, J = 8.7 Hz, 1H), 8.51-8.46 (m, 1H), 7.95-7.88 (m, |
| | | | | 2H), 7.79-7.73 (m, 1H), 7.73-7.66 (m, 1H), 7.37-7.30 (m, 2H), 7.30- |
| | | | | 7.23 (m, 1H), 7.15-7.09 (m, 1H), 5.97 (s, 1H), 4.42-4.29 (m, 1H), 4.20 |
| | | | | (t, J = 8.3 Hz, 2H), 3.99-3.91 (m, 1H), 3.88 (s, 3H), 3.82-3.73 (m, 3H), |
| | | | | 3.59 (t, J = 6.7 Hz, 2H), 3.38-3.32 (m, 4H), 3.27-3.17 (m, 8H), 2.97 (d, |
| | | | | J = 4.9 Hz, 3H), 2.72-2.64 (m, 2H), 2.17-1.99 (m, 4H), 1.60-1.40 (m, 2H) |
| I-250 | US | MM | 833.5 | 10.51 (s, 1H), 8.88 (d, J = 4.7 Hz, 1H), 8.85-8.80 (m, 2H), 7.96 (s, 1H), |
| | | | | 7.85 (d, J = 7.9 Hz, 2H), 7.74-7.68 (m, 1H), 7.65 (d, J = 8.3 Hz, 1H), |
| | | | | 7.55 (d, J = 2.0 Hz, 1H), 7.39 (dd, J = 8.3, 2.0 Hz, 1H), 7.35-7.29 (m, |
| | | | | 1H), 6.03 (s, 1H), 4.99-4.77 (m, 1H), 4.22-4.12 (m, 2H), 3.80-3.72 (m, |
| | | | | 1H), 3.71-3.60 (m, 6H), 3.57-3.52 (m, 1H), 3.28-3.24 (m, 1H), 3.11-3.02 |
| | | | | (m, 6H), 2.98 (d, J = 4.8 Hz, 3H), 2.78-2.71 (m, 2H), 1.77-1.65 (m, 4H), |
| | | | | 1.26-1.20 (m, 1H), 1.03-0.92 (m, 1H) |
| I-29 | RX | MM | 850.1 | 10.52 (s, 1H), 8.75 (d, J = 4.4 Hz, 1H), 8.60 (d, J = 2.0 Hz, 1H), 7.90 (s, |
| | | | | 1H), 7.84-7.77 (m, 2H), 7.75-7.63 (m, 3H), 7.56 (s, 1H), 7.39 (dd, |
| | | | | J = 8.4, 2.0 Hz, 1H), 7.34-7.28 (m, 2H), 5.92 (s, 1H), 4.16 (t, J = 8.4 Hz, |
| | | | | 2H), 4.00-3.83 (m, 1H), 3.80-3.56 (m, 5H), 3.47-3.37 (m, 4H), 3.30- |
| | | | | 3.26 (m, 2H), 3.23-3.14 (m, 2H), 3.01-2.87 (m, 4H), 2.79-2.69 |
| | | | | (m, 2H), 2.08-1.97 (m, 2H), 0.85-0.74 (m, 2H), 0.59-0.53 (m, 2H) |
| I-319 | PR | QV | 831.4 | 10.92 (s, 1H), 8.88 (d, J = 4.8 Hz, 1H), 8.58 (d, J = 2.0 Hz, 1H), 8.22 (s, |
| | | | | 2H) (FA), 7.96 (s, 1H), 7.85-7.64 (m, 4H), 7.53 (d, J = 8.0 Hz, 1H), |
| | | | | 7.42-7.23 (m, 4H), 6.01 (s, 1H), 5.03-4.73 (m, 1H), 4.27 (dd, J = |
| | | | | 12.4, 5.2 Hz, 1H), 4.15 (t, J = 8.4 Hz, 2H), 3.69 (s, 2H), 3.59-3.50 (m, |
| | | | | 2H), 3.43 (t, J = 8.4 Hz, 2H), 3.31-3.18 (m, 2H), 3.12-3.01 (m, 5H), |
| | | | | 2.97 (d, J = 4.8 Hz, 3H), 2.87-2.71 (m, 1H), 2.59-2.56 (m, 1H), 2.40- |
| | | | | 2.27 (m, 1H), 2.06-1.95 (m, 1H), 1.80-1.63 (m, 4H), 1.29-1.17 |
| | | | | (m, 1H), 1.04-0.91 (m, 1H) |
| I-348 | SA | MM | 858.4 | 10.52 (s, 1H), 9.05 (d, J = 8.8 Hz, 1H), 8.59 (s, 1H), 7.93 (s, 1H), 7.90- |
| | | | | 7.85 (m, 1H), 7.82-7.76 (m, 1H), 7.75-7.67 (m, 2H), 7.64 (d, J = |
| | | | | 8.0 Hz, 1H), 7.55 (d, J = 2.0 Hz, 1H), 7.39 (dd, J = 8.0, 2.0 Hz, 1H), 7.36- |
| | | | | 7.28 (m, 2H), 5.96 (s, 1H), 4.42-4.28 (m, 1H), 4.20 (t, J = 8.4 Hz, |
| | | | | 2H), 3.83-3.72 (m, 2H), 3.71-3.51 (m, 5H), 3.47 (t, J = 8.4 Hz, 2H), |
| | | | | 3.30-3.23 (m, 2H), 3.21 (s, 3H), 3.11-3.01 (m, 4H), 2.97 (d, J = 4.8 |
| | | | | Hz, 3H), 2.80-2.71 (m, 2H), 2.19-1.99 (m, 2H), 1.82-1.63 (m, 4H), |
| | | | | 1.59-1.36 (m, 2H) |
| I-351 | PR | PS | 813.5 | 10.48 (s, 1H), 8.88 (d, J = 4.8 Hz, 1H), 8.58 (d, J = 2.0 Hz, 1H), 8.43 (d, |
| | | | | J = 2.0 Hz, 1H), 8.27 (s, 2H) (FA), 7.96 (s, 1H), 7.85-7.64 (m, 5H), |
| | | | | 7.35-7.23 (m, 2H), 6.00 (s, 1H), 5.04-4.69 (m, 1H), 4.13 (t, J = 8.4 |
| | | | | Hz, 2H), 3.93-3.77 (m, 1H), 3.69 (s, 2H), 3.66-3.50 (m, 3H), 3.42 (t, |
| | | | | J = 8.4 Hz, 2H), 3.36-3.20 (m, 2H), 3.15-3.01 (m, 5H), 2.96 (d, J = |
| | | | | 4.8 Hz, 3H), 2.89-2.64 (m, 2H), 2.43 (s, 3H), 1.81-1.62 (m, 4H), 1.31- |
| | | | | 1.15 (m, 1H), 1.08-0.87 (m, 1H) |
| I-352 | PR | UO | 829.4 | 10.46 (s, 1H), 8.88 (d, J = 4.8 Hz, 1H), 8.58 (d, J = 2.0 Hz, 1H), 8.20 (d, |
| | | | | J = 2.4 Hz, 1H), 7.96 (s, 1H), 7.84-7.67 (m, 5H), 7.36-7.25 (m, 2H), |
| | | | | 6.00 (s, 1H), 5.00-4.75 (m, 1H), 4.14 (t, J = 8.0 Hz, 2H), 3.95 (s, 3H), |
| | | | | 3.72-3.60 (m, 4H), 3.56-3.37 (m, 6H), 3.10-3.01 (m, 5H), 2.97 (d, |
| | | | | J = 4.8 Hz, 3H), 2.70 (t, J = 6.8 Hz, 2H), 1.83-1.65 (m, 4H), 1.31- |
| | | | | 1.15 (m, 1H), 1.05-0.90 (m, 1H) |
| I-358 | UG | PW | 863.4 | 10.34 (s, 1H), 8.88 (d, J = 4.8 Hz, 1H), 8.60 (d, J = 2.4 Hz, 1H), 8.24 (s, |
| | | | | 0.7H) (FA), 7.96 (s, 1H), 7.85-7.62 (m, 4H), 7.46-7.15 (m, 5H), 6.01 |
| | | | | (s, 1H), 5.02-4.71 (m, 1H), 4.14 (t, J = 8.4 Hz, 2H), 3.93-3.74 (m, |
| | | | | 5H), 3.73-3.68 (m, 2H), 3.61 (t, J = 6.4 Hz, 2H), 3.58-3.48 (m, 2H), |
| | | | | 3.43 (t, J = 8.4 Hz, 2H), 3.37-3.25 (m, 2H), 3.22-3.13 (m, 2H), 3.10- |
| | | | | 3.01 (m, 1H), 2.97 (d, J = 4.8 Hz, 3H), 2.69 (t, J = 6.4 Hz, 2H), 2.11- |
| | | | | 1.96 (m, 2H), 1.31-1.17 (m, 1H), 1.06-0.91 (m, 1H) |
| I-464 | UU | MM | 833.3 | 10.51 (s, 1H), 8.86 (d, J = 4.7 Hz, 1H), 8.04 (d, J = 8.7 Hz, 1H), 7.97 (s, |
| | | | | 1H), 7.86 (dd, J = 7.3, 1.7 Hz, 1H), 7.78-7.66 (m, 2H), 7.65 (d, J = 8.2 |
| | | | | Hz, 1H), 7.56 (d, J = 2.0 Hz, 1H), 7.43-7.27 (m, 3H), 6.02 (s, 1H), 5.00- |
| | | | | 4.78 (m, 1H), 4.18 (t, J = 8.5 Hz, 2H), 4.02 (s, 2H), 3.80-3.71 (m, 1H), |
| | | | | 3.68-3.52 (m, 3H), 3.44 (t, J = 8.5 Hz, 2H), 3.29-3.11 (m, 6H), 3.08-3.01 |
| | | | | (m, 1H), 2.97 (d, J = 4.9 Hz, 3H), 2.78-2.71 (m, 2H), 1.84-1.67 (m, 4H), |
| | | | | 1.28-1.16 (m, 1H), 1.05-0.90 (m, 1H) |
| I-465 | VB | MM | 833.3 | 10.51 (s, 1H), 8.97-8.92 (m, 2H), 8.84 (d, J = 4.7 Hz, 1H), 7.97 (s, 1H), |
| | | | | 7.79 (d, J = 8.1 Hz, 1H), 7.76-7.70 (m, 1H), 7.65 (d, J = 8.2 Hz, 1H), |
| | | | | 7.56 (d, J = 2.0 Hz, 1H), 7.39 (dd, J = 8.3, 2.0 Hz, 1H), 7.33 (t, J = 7.8 |
| | | | | Hz, 1H), 7.07 (d, J = 7.6 Hz, 1H), 6.01 (s, 1H), 5.02-4.77 (m, 1H), 4.16 |
| | | | | (t, J = 8.4 Hz, 2H), 3.86 (s, 2H), 3.80-3.72 (m, 1H), 3.66-3.48 (m, 3H), |
| | | | | 3.30-3.25 (m, 4H), 3.25-3.18 (m, 4H), 3.06-3.02 (m, 1H), 2.97 (d, J = |
| | | | | 4.9 Hz, 3H), 2.78-2.70 (m, 2H), 1.80-1.66 (m, 4H), 1.26-1.18 (m, 1H), |
| | | | | 1.02-0.91 (m, 1H) |
| I-466 | UY | MM | 833.3 | 10.51 (s, 1H), 8.99-8.93 (m, 1H), 8.88-8.83 (m, 1H), 8.72-8.65 (m, 1H), |
| | | | | 8.00-7.94 (m, 1H), 7.86-7.78 (m, 1H), 7.74-7.69 (m, 1H), 7.68-7.61 (m, |
| | | | | 1H), 7.56 (d, J = 2.0 Hz, 1H), 7.43-7.29 (m, 3H), 6.02-5.97 (m, 1H), |
| | | | | 4.99-4.76 (m, 1H), 4.21-4.10 (m, 2H), 3.86-3.71 (m, 3H), 3.68-3.50 (m, |
| | | | | 3H), 3.49-3.41 (m, 2H), 3.30-3.24 (m, 2H), 3.15 (s, 4H), 3.07-3.02 (m, |
| | | | | 1H), 3.00-2.91 (m, 3H), 2.79-2.71 (m, 2H), 1.83-1.65 (m, 4H), 1.28- |
| | | | | 1.16 (m, 1H), 1.04-0.90 (m, 1H) |
| I-480 | SG | SH | 817.3 | 10.96 (s, 1H), 8.97-8.87 (m, 1H), 7.97-7.93 (m, 2H), 7.72-7.63 (m, 2H), |
| | | | | 7.58-7.54 (m, 1H), 7.52-7.50 (m, 1H), 7.46-7.42 (m, 1H), 7.39-7.35 (m, |
| | | | | 1H), 7.26-7.21 (m, 2H), 6.94-6.89 (m, 1H), 5.99 (s, 1H), 5.00-4.75 (m, |
| | | | | 1H), 4.28 (dd, J = 12.4, 5.0 Hz, 1H), 4.12 (t, J = 8.5 Hz, 2H), 3.80-3.70 |
| | | | | (m, 4H), 3.67-3.57 (m, 2H), 3.40 (t, J = 8.5 Hz, 2H), 3.11-3.01 (m, 1H), |
| | | | | 2.96 (d, J = 4.9 Hz, 3H), 2.88-2.75 (m, 1H), 2.61-2.51 (m, 3H), 2.40- |
| | | | | 2.26 (m, 1H), 2.08-1.99 (m, 1H), 1.90-1.77 (m, 4H), 1.29-1.17 (m, 1H), |
| | | | | 1.05-0.92 (m, 1H) |
| I-502 | UG | QV | 867.3 | 10.91 (s, 1H), 8.88 (d, J = 4.8 Hz, 1H), 8.60 (s, 1H), 7.96 (s, 1H), 7.88 |
| | | | | 7.65 (m, 4H), 7.58 (d, J = 8.0 Hz, 1H), 7.43-7.24 (m, 4H), 6.01 (s, |
| | | | | 1H), 5.07-4.71 (m, 1H), 4.30 (dd, J = 12.4, 5.2 Hz, 1H), 4.15 (t, J = 8.4 |
| | | | | Hz, 2H), 4.00-3.54 (m, 6H), 3.47-3.39 (m, 4H), 3.25-3.11 (m, 2H), |
| | | | | 3.11-2.91 (m, 4H), 2.89-2.68 (m, 1H), 2.65-2.55 (m, 1H), 2.43- |
| | | | | 2.20 (m, 1H), 2.13-1.94 (m, 3H), 1.33-1.14 (m, 1H), 1.10-0.86 (m, 1H) |
| I-57 | UD | MM | 878.3 | 10.48 (s, 1H), 8.77 (d, J = 4.2 Hz, 1H), 8.61 (d, J = 2.1 Hz, 1H), 7.91 (s, |
| | | | | 1H), 7.87-7.72 (m, 3H), 7.72-7.62 (m, 2H), 7.58 (d, J = 2.0 Hz, 1H), |
| | | | | 7.41 (dd, J = 8.2, 2.1 Hz, 1H), 7.37-7.28 (m, 2H), 5.96-5.90 (m, 1H), |
| | | | | 4.17 (t, J = 8.5 Hz, 2H), 3.82-3.73 (m, 1H), 3.70 (s, 2H), 3.66-3.58 (m, |
| | | | | 1H), 3.47 (t, J = 8.4 Hz, 2H), 3.38-3.03 (m, 4H), 3.02-2.88 (m, 4H), |
| | | | | 2.80-2.66 (m, 4H), 2.62-2.53 (m, 2H), 1.92-1.79 (m, 2H), 1.77-1.54 (m, |
| | | | | 4H), 0.86-0.71 (m, 2H), 0.63-0.51 (m, 2H) |
| I-63 | PR | RI | 812.4 | 10.37 (s, 1H), 8.89 (d, J = 4.7 Hz, 1H), 8.58 (d, J = 2.2 Hz, 1H), 8.30 (s, |
| | | | | 1H), 7.96 (s, 1H), 7.82-7.68 (m, 4H), 7.38-7.21 (m, 5H), 6.02 (s, 1H), |
| | | | | 5.00-4.77 (m, 1H), 4.15 (t, J = 8.5 Hz, 2H), 3.85-3.76 (m, 1H), 3.68 (s, |
| | | | | 2H), 3.60-3.49 (m, 3H), 3.47-3.37 (m, 5H), 3.05 (s, 4H), 2.97 (d, J = 4.9 |
| | | | | Hz, 3H), 2.83-2.68 (m, 2H), 2.22 (s, 3H), 1.81-1.63 (m, 4H), 1.24-1.16 |
| | | | | (m, 1H), 1.02-0.90 (m, 1H) |
| I-630 | PU | MM | 831.3 | 10.49 (s, 1H), 8.88 (d, J = 4.7 Hz, 1H), 8.56-8.49 (m, 1H), 7.96 (s, 1H), |
| | | | | 7.79-7.59 (m, 5H), 7.55 (d, J = 2.0 Hz, 1H), 7.38 (dd, J = 8.2, 2.0 Hz, |
| | | | | 1H), 7.34-7.23 (m, 2H), 6.02 (s, 1H), 5.04-4.74 (m, 1H), 4.15 (t, J = 8.4 |
| | | | | Hz, 2H), 3.82-3.70 (m, 1H), 3.69-3.49 (m, 3H), 3.48-3.39 (m, 3H), 3.21- |
| | | | | 3.16 (m, 1H), 3.09-3.01 (m, 1H), 2.97 (d, J = 4.9 Hz, 3H), 2.84-2.69 (m, |
| | | | | 4H), 2.66-2.56 (m, 1H), 2.03-1.91 (m, 2H), 1.70-1.44 (m, 6H), 1.29- |
| | | | | 1.17 (m, 1H), 1.08-0.88 (m, 1H) |
| I-64 | PR | PW | 828.3 | 10.32 (s, 1H), 8.88 (d, J = 4.7 Hz, 1H), 8.59 (d, J = 2.2 Hz, 1H), 7.96 (s, |
| | | | | 1H), 7.84-7.65 (m, 4H), 7.42-7.23 (m, 4H), 7.16 (d, J = 8.5 Hz, 1H), |
| | | | | 6.02 (s, 1H), 5.02-4.72 (m, 1H), 4.15 (t, J = 8.4 Hz, 2H), 3.85 (s, 3H), |
| | | | | 3.69 (s, 2H), 3.60 (t, J = 6.6 Hz, 2H), 3.54-3.34 (m, 6H), 3.12-3.01 (m, |
| | | | | 5H), 2.98 (d, J = 4.8 Hz, 3H), 2.69 (t, J = 6.7 Hz, 2H), 1.79-1.61 (m, |
| | | | | 4H), 1.28-1.19 (m, 1H), 1.08-0.91 (m, 1H) |
| I-651 | VI | VK | 882.4 | 10.52 (s, 1H), 9.07 (d, J = 8.4 Hz, 1H), 8.61 (d, J = 2.0 Hz, 1H), 7.98- |
| | | | | 7.88 (m, 2H), 7.85-7.78 (m, 1H), 7.77-7.65 (m, 3H), 7.57 (s, 1H), |
| | | | | 7.43-7.37 (m, 1H), 7.31 (d, J = 4.4 Hz, 2H), 5.96 (s, 1H), 5.05-4.80 |
| | | | | (m, 1H), 4.67-4.49 (m, 1H), 4.20 (t, J = 8.4 Hz, 2H), 4.00-3.57 (m, |
| | | | | 7H), 3.52-3.39 (m, 4H), 3.22-3.11 (m, 2H), 2.97 (d, J = 4.8 Hz, 3H), |
| | | | | 2.82-2.71 (m, 2H), 2.30-2.11 (m, 3H), 2.10-1.98 (m, 2H), 1.92- |
| | | | | 1.77 (m, 1H), 1.49-1.34 (m, 1H) |
| I-652 | VL | VK | 882.4 | 10.53 (s, 1H), 8.94 (d, J = 6.6 Hz, 1H), 8.60 (d, J = 2.2 Hz, 1H), 7.91- |
| | | | | 7.85 (m, 3H), 7.84-7.77 (m, 1H), 7.74-7.67 (m, 3H), 7.57 (s, 1H), 7.43- |
| | | | | 7.27 (m, 3H), 5.97 (s, 1H), 5.39-5.17 (m, 1H), 4.65 (q, J = 7.0 Hz, |
| | | | | 1H), 4.19 (t, J = 8.2 Hz, 2H), 3.94-3.84 (m, 1H), 3.81-3.58 (m, 6H), |
| | | | | 3.46 (t, J = 8.6 Hz, 2H), 3.44-3.35 (m, 3H), 3.18 (d, J = 7.7 Hz, 2H), |
| | | | | 2.96 (d, J = 4.8 Hz, 3H), 2.80-2.70 (m, 2H), 2.67-2.52 (m, 2H), 2.49- |
| | | | | 2.29 (m, 2H), 2.07-1.99 (m, 2H) |
| I-654 | VP | MM | 859.4 | 10.49 (s, 1H), 8.89 (d, J = 4.7 Hz, 1H), 8.50 (s, 1H), 7.96 (s, 1H), 7.74 |
| | | | | (dd, J = 6.7, 2.1 Hz, 1H), 7.72-7.60 (m, 4H), 7.55 (d, J = 2.0 Hz, 1H), |
| | | | | 7.39 (dd, J = 8.2, 2.0 Hz, 1H), 7.35-7.23 (m, 2H), 6.02 (s, 1H), 5.04-4.73 |
| | | | | (m, 1H), 4.15 (t, J = 8.4 Hz, 2H), 3.81-3.70 (m, 1H), 3.68-3.52 (m, 3H), |
| | | | | 3.44 (t, J = 8.4 Hz, 2H), 3.28-3.24 (m, 2H), 3.10-3.02 (m, 1H), 2.98 (d, |
| | | | | J = 4.9 Hz, 3H), 2.80-2.72 (m, 2H), 2.63-2.54 (m, 2H), 1.78-1.64 (m, |
| | | | | 2H), 1.60-1.42 (m, 5H), 1.37-1.07 (m, 7H), 1.05-0.89 (m, 1H) |
| I-655 | PV | ME | 910.3 | 10.47 (s, 1H), 8.89 (d, J = 4.7 Hz, 1H), 8.60 (d, J = 2.1 Hz, 1H), 8.26 (s, |
| | | | | 1H), 7.96 (s, 1H), 7.82 (dd, J = 8.1, 2.2 Hz, 1H), 7.78-7.73 (m, 2H), |
| | | | | 7.73-7.66 (m, 1H), 7.43-7.39 (m, 2H), 7.37-7.26 (m, 2H), 6.01 (s, 1H), |
| | | | | 5.00-4.76 (m, 1H), 4.26-4.07 (m, 3H), 3.79-3.67 (m, 3H), 3.65-3.57 (m, |
| | | | | 1H), 3.54-3.41 (m, 4H), 3.12-3.00 (m, 2H), 2.97 (d, J = 4.9 Hz, 3H), |
| | | | | 2.82-2.69 (m, 4H), 2.58-2.53 (m, 2H), 2.41 (s, 3H), 1.91-1.78 (m, 2H), |
| | | | | 1.75-1.52 (m, 4H), 1.30-1.16 (m, 1H), 1.05-0.90 (m, 1H) |
| I-656 | PV | TJ | 914.3 | 10.58 (s, 1H), 8.89 (d, J = 4.7 Hz, 1H), 8.60 (d, J = 2.2 Hz, 1H), 7.96 (s, |
| | | | | 1H), 7.82 (dd, J = 8.2, 2.1 Hz, 1H), 7.78-7.74 (m, 2H), 7.73-7.68 (m, |
| | | | | 1H), 7.53 (dd, J = 8.9, 1.8 Hz, 1H), 7.49-7.44 (m, 1H), 7.35-7.26 (m, |
| | | | | 2H), 6.01 (s, 1H), 4.99-4.77 (m, 1H), 4.23-4.09 (m, 3H), 3.87-3.77 (m, |
| | | | | 1H), 3.72-3.59 (m, 3H), 3.49-3.40 (m, 3H), 3.30-3.21 (m, 1H), 3.17- |
| | | | | 3.00 (m, 2H), 2.96 (d, J = 4.9 Hz, 3H), 2.79-2.69 (m, 4H), 2.56-2.52 (m, |
| | | | | 2H), 1.87-1.77 (m, 2H), 1.75-1.63 (m, 3H), 1.62-1.52 (m, 1H), 1.26- |
| | | | | 1.15 (m, 1H), 1.04-0.89 (m, 1H) |
| I-657 | PV | SI | 894.4 | 10.46 (s, 1H), 8.89 (d, J = 4.8 Hz, 1H), 8.60 (d, J = 2.0 Hz, 1H), 8.31 (s, |
| | | | | 0.4H) (FA), 7.96 (s, 1H), 7.89-7.65 (m, 4H), 7.41-7.16 (m, 4H), 6.01 |
| | | | | (s, 1H), 5.02-4.71 (m, 1H), 4.28-4.06 (m, 3H), 3.92-3.79 (m, 1H), |
| | | | | 3.69 (s, 2H), 3.61-3.43 (m, 5H), 3.17-3.01 (m, 3H), 2.97 (d, J = 4.8 |
| | | | | Hz, 3H), 2.89-2.63 (m, 4H), 2.58-2.52 (m, 2H), 2.12 (s, 3H), 1.93- |
| | | | | 1.47 (m, 5H), 1.33-1.13 (m, 1H), 1.09-0.88 (m, 1H) |
| I-659 | PV | RI | 876.3 | 10.39 (s, 1H), 8.89 (d, J = 4.7 Hz, 1H), 8.60 (d, J = 2.1 Hz, 1H), 7.96 (s, |
| | | | | 1H), 7.81 (dd, J = 8.2, 2.2 Hz, 1H), 7.78-7.68 (m, 3H), 7.38-7.23 (m, |
| | | | | 5H), 6.01 (s, 1H), 5.00-4.76 (m, 1H), 4.24-4.10 (m, 3H), 3.87-3.76 (m, |
| | | | | 1H), 3.68 (s, 2H), 3.58-3.48 (m, 2H), 3.44 (t, J = 8.5 Hz, 2H), 3.29-3.21 |
| | | | | (m, 1H), 3.11-3.00 (m, 2H), 2.96 (d, J = 4.9 Hz, 3H), 2.85-2.65 (m, 4H), |
| | | | | 2.56-2.52 (m, 2H), 2.22 (s, 3H), 1.88-1.78 (m, 2H), 1.73-1.54 (m, 4H), |
| | | | | 1.23-1.15 (m, 1H), 1.04-0.89 (m, 1H) |
| I-660 | PV | PW | 892.4 | 10.35 (s, 1H), 8.89 (d, J = 4.7 Hz, 1H), 8.61 (d, J = 2.1 Hz, 1H), 7.96 (s, |
| | | | | 1H), 7.88-7.69 (m, 4H), 7.42-7.30 (m, 4H), 7.17 (d, J = 8.6 Hz, 1H), |
| | | | | 6.02 (s, 1H), 5.00-4.78 (m, 1H), 4.16 (t, J = 8.5 Hz, 2H), 3.85 (s, 3H), |
| | | | | 3.70 (s, 2H), 3.61 (t, J = 6.7 Hz, 2H), 3.45 (t, J = 8.5 Hz, 2H), 3.40-3.32 |
| | | | | (m, 6H), 3.07-3.03 (m, 1H), 2.97 (d, J = 4.9 Hz, 3H), 2.78-2.66 (m, 4H), |
| | | | | 1.89-1.81 (m, 2H), 1.72-1.62 (m, 4H), 1.30-1.16 (m, 1H), 1.03-0.93 |
| | | | | (m, 1H) |
| I-661 | SK | MM | 914.3 | 10.52 (s, 1H), 8.76 (d, J = 4.8 Hz, 1H), 8.61 (s, 1H), 7.98 (s, 1H), 7.90- |
| | | | | 7.56 (m, 6H), 7.41 (dd, J = 8.4, 2.0 Hz, 1H), 7.14 (dd, J = 10.0, 2.4 |
| | | | | Hz, 1H), 5.98 (s, 1H), 5.00-4.70 (m, 1H), 4.22-4.18 (m, 3H), 3.83- |
| | | | | 3.57 (m, 6H), 3.51-3.40 (m, 4H), 3.16-3.00 (m, 2H), 2.97 (d, J = 4.8 |
| | | | | Hz, 3H), 2.83-2.66 (m, 4H), 1.93-1.49 (m, 6H), 1.30-1.11 (m, 1H), |
| | | | | 1.05-0.90 (m, 1H) |
| I-668 | PV | VN | 910.4 | 10.41 (s, 1H), 8.89 (d, J = 4.8 Hz, 1H), 8.63-8.58 (m, 1H), 7.96 (s, 1H), |
| | | | | 7.86-7.68 (m, 4H), 7.52-7.47 (m, 1H), 7.40-7.26 (m, 3H), 6.02 (s, 1H), |
| | | | | 4.99-4.76 (m, 1H), 4.31-4.21 (m, 1H), 4.16 (t, J = 8.5 Hz, 2H), 3.85-3.74 |
| | | | | (m, 1H), 3.69 (s, 2H), 3.56-3.50 (m, 1H), 3.45 (t, J = 8.5 Hz, 2H), 3.31- |
| | | | | 3.28 (m, 2H), 3.23-3.16 (m, 2H), 3.14-3.01 (m, 2H), 2.97 (d, J = 4.9 Hz, |
| | | | | 3H), 2.81-2.64 (m, 4H), 2.21 (s, 3H), 1.93-1.80 (m, 2H), 1.78-1.62 (m, |
| | | | | 3H), 1.61-1.54 (m, 1H), 1.26-1.20 (m, 1H), 1.03-0.92 (m, 1H) |
| I-669 | PV | VG | 894.4 | 10.40 (s, 1H), 8.90 (d, J = 4.6 Hz, 1H), 8.64-8.60 (m, 1H), 7.96 (s, 1H), |
| | | | | 7.84-7.81 (m, 1H), 7.79-7.75 (m, 2H), 7.74-7.69 (m, 1H), 7.40-7.34 (m, |
| | | | | 1H), 7.33-7.24 (m, 3H), 6.02 (s, 1H), 4.99-4.77 (m, 1H), 4.29-4.22 (m, |
| | | | | 1H), 4.16 (t, J = 8.4 Hz, 2H), 3.84-3.74 (m, 1H), 3.70 (s, 2H), 3.57-3.42 |
| | | | | (m, 3H), 3.31-3.20 (m, 3H), 3.13-3.02 (m, 2H), 2.97 (d, J = 4.9 Hz, 3H), |
| | | | | 2.80-2.70 (m, 4H), 2.22 (s, 3H), 1.93-1.80 (m, 2H), 1.79-1.56 (m, 5H), |
| | | | | 1.26-1.22 (m, 1H), 1.04-0.92 (m, 1H) |
| I-67 | UG | MM | 868.3 | 10.52 (s, 1H), 8.88 (d, J = 4.7 Hz, 1H), 8.66 (s, 1H), 7.96 (s, 1H), 7.90- |
| | | | | 7.82 (m, 1H), 7.80-7.66 (m, 4H), 7.59-7.54 (m, 1H), 7.40 (dd, J = 8.2, |
| | | | | 2.1 Hz, 1H), 7.35-7.26 (m, 2H), 6.01 (s, 1H), 5.00-4.76 (m, 1H), 4.15 (t, |
| | | | | J = 8.5 Hz, 2H), 4.03-3.72 (m, 5H), 3.71-3.58 (m, 2H), 3.55-3.35 (m, |
| | | | | 6H), 3.11-3.01 (m, 2H), 2.97 (d, J = 4.9 Hz, 3H), 2.80-2.71 (m, 2H), |
| | | | | 2.14-2.01 (m, 2H), 1.24-1.19 (m, 1H), 1.05-0.90 (m, 1H) |
| I-670 | VC | MM | 922.5 | 10.51 (s, 1H), 9.05 (d, J = 8.8 Hz, 1H), 8.61 (d, J = 2.0 Hz, 1H), 7.93 (s, |
| | | | | 1H), 7.88 (dd, J = 7.2, 1.6 Hz, 1H), 7.86-7.78 (m, 1H), 7.80-7.73 (m, |
| | | | | 1H), 7.73-7.61 (m, 2H), 7.58 (d, J = 2.0 Hz, 1H), 7.41 (dd, J = 8.0, 2.0 |
| | | | | Hz, 1H), 7.39-7.28 (m, 2H), 5.97 (s, 1H), 4.42-4.28 (m, 1H), 4.26- |
| | | | | 4.15 (m, 3H), 3.84-3.73 (m, 2H), 3.69 (s, 2H), 3.66-3.57 (m, 1H), |
| | | | | 3.52-3.44 (m, 3H), 3.21 (s, 3H), 3.17-3.05 (m, 1H), 2.97 (d, J = 4.8 |
| | | | | Hz, 3H), 2.80-2.66 (m, 4H), 2.60-2.53 (m, 3H), 2.17-1.98 (m, 2H), |
| | | | | 1.91-1.77 (m, 2H), 1.76-1.63 (m, 3H), 1.61-1.36 (m, 3H) |
| I-686 | QE | MM | 901.3 | 10.49 (s, 1H), 8.87 (d, J = 4.6 Hz, 1H), 7.95 (s, 1H), 7.70-7.59 (m, 2H), |
| | | | | 7.58-7.48 (m, 2H), 7.39 (dd, J = 8.2, 2.0 Hz, 1H), 7.11 (t, J = 7.7 Hz, |
| | | | | 1H), 6.76 (d, J = 7.6 Hz, 1H), 6.00 (s, 1H), 5.01-4.74 (m, 1H), 4.14 (t, |
| | | | | J = 8.5 Hz, 2H), 3.80-3.69 (m, 1H), 3.68-3.50 (m, 3H), 3.19-3.00 (m, 5H), |
| | | | | 2.96 (d, J = 4.9 Hz, 3H), 2.85-2.69 (m, 3H), 2.47-2.31 (m, 3H), 2.25- |
| | | | | 1.93 (m, 2H), 1.81-1.71 (m, 2H), 1.65-1.18 (m, 12H), 1.18-0.98 (m, 3H), |
| | | | | 0.96-0.81 (m, 1H) |
| I-697 | RM | MM | 916.3 | 10.51 (s, 1H), 8.89 (d, J = 4.7 Hz, 1H), 7.94 (s, 1H), 7.70-7.60 (m, 2H), |
| | | | | 7.57 (d, J = 2.0 Hz, 1H), 7.49 (d, J = 8.0 Hz, 1H), 7.41 (dd, J = 8.2, 2.1 |
| | | | | Hz, 1H), 7.09 (t, J = 7.8 Hz, 1H), 6.72 (d, J = 7.6 Hz, 1H), 6.00 (s, 1H), |
| | | | | 4.97-4.78 (m, 1H), 4.51-4.38 (m, 1H), 4.13 (t, J = 8.6 Hz, 2H), 3.78-3.73 |
| | | | | (m, 1H), 3.67-3.54 (m, 2H), 3.12 (t, J = 8.4 Hz, 2H), 3.08-2.98 (m, 3H), |
| | | | | 2.96 (d, J = 4.9 Hz, 3H), 2.94-2.83 (m, 3H), 2.79-2.70 (m, 3H), 2.66- |
| | | | | 2.58 (m, 1H), 2.49-2.36 (m, 6H), 2.30-2.21 (m, 1H), 1.83-1.65 (m, 3H), |
| | | | | 1.62-1.49 (m, 3H), 1.45-1.32 (m, 2H), 1.32-1.11 (m, 4H), 1.02-0.91 (m, 1H) |
| I-700 | VM | MM | 926.4 | 10.52 (s, 1H), 8.80 (d, J = 4.8 Hz, 1H), 7.96 (s, 1H), 7.77-7.67 (m, 2H), |
| | | | | 7.64 (d, J = 8.4 Hz, 1H), 7.55 (d, J = 2.0 Hz, 1H), 7.39 (dd, J = 8.4, 2.0 |
| | | | | Hz, 1H), 7.16 (t, J = 8.0 Hz, 1H), 6.97 (d, J = 7.6 Hz, 1H), 5.95 (s, 1H), |
| | | | | 5.00-4.76 (m, 1H), 4.18 (t, J = 8.4 Hz, 2H), 3.82-3.52 (m, 6H), 3.32- |
| | | | | 3.26 (m, 2H), 3.22 (t, J = 8.4 Hz, 2H), 3.10-2.99 (m, 2H), 2.98-2.88 |
| | | | | (m, 4H), 2.87-2.62 (m, 7H), 2.57-2.52 (m, 1H), 2.46-2.35 (m, 1H), |
| | | | | 1.89-1.75 (m, 2H), 1.55-1.34 (m, 8H), 1.28-1.16 (m, 1H), 1.02- |
| | | | | 0.89 (m, 1H) |
| I-706 | VF | MM | 889.3 | 10.52 (s, 1H), 8.88 (d, J = 4.7 Hz, 1H), 7.95 (s, 1H), 7.71-7.63 (m, 2H), |
| | | | | 7.59-7.55 (m, 2H), 7.42-7.39 (m, 1H), 7.17-7.11 (m, 1H), 6.84 (d, J = |
| | | | | 7.6 Hz, 1H), 6.00 (s, 1H), 4.99-4.78 (m, 1H), 4.13 (t, J = 8.6 Hz, 2H), |
| | | | | 3.80-3.72 (m, 1H), 3.67-3.57 (m, 3H), 3.53 (s, 2H), 3.48-3.42 (m, 1H), |
| | | | | 3.39-3.35 (m, 2H), 3.16 (t, J = 8.5 Hz, 2H), 3.07-2.90 (m, 8H), 2.83-2.71 |
| | | | | (m, 3H), 2.65-2.53 (m, 3H), 2.35-2.22 (m, 5H), 1.76-1.65 (m, 1H), 1.58- |
| | | | | 1.47 (m, 1H), 1.27-1.20 (m, 1H), 1.03-0.92 (m, 1H) |
| I-711 | QF | MS | 916.4 | 10.45 (s, 1H), 8.87 (d, J = 4.7 Hz, 1H), 7.95 (s, 1H), 7.68 (q, J = 5.0 Hz, |
| | | | | 1H), 7.57 (d, J = 8.0 Hz, 1H), 7.44 (d, J = 1.6 Hz, 1H), 7.36 (d, J = 1.6 |
| | | | | Hz, 1H), 7.14 (t, J = 7.8 Hz, 1H), 6.84 (d, J = 7.6 Hz, 1H), 5.99 (s, 1H), |
| | | | | 5.00-4.80 (m, 1H), 4.13 (t, J = 8.7 Hz, 2H), 3.86-3.80 (m, 1H), 3.60- |
| | | | | 3.45 (m, 5H), 3.35-3.27 (m, 3H), 3.17 (t, J = 8.6 Hz, 2H), 3.07-3.01 (m, |
| | | | | 1H), 2.95 (d, J = 4.9 Hz, 3H), 2.92-2.76 (m, 2H), 2.75-2.63 (m, 1H), |
| | | | | 2.74-2.64 (m, 5H), 2.36-2.24 (m, 1H), 2.23 (s, 3H), 2.18-2.08 (m, 1H), |
| | | | | 1.88-1.64 (m, 2H), 1.47-1.35 (m, 8H), 1.27-1.19 (m, 1H), 1.05-0.95 |
| | | | | (m, 1H) |
| I-712 | QF | TQ | 920.3 | 10.58 (s, 1H), 8.87 (d, J = 4.8 Hz, 1H), 7.95 (s, 1H), 7.74-7.38 (m, 4H), |
| | | | | 7.14 (t, J = 8.0 Hz, 1H), 6.84 (dd, J = 8.0, 2.8 Hz, 1H), 5.99 (d, J = 4.8 |
| | | | | Hz, 1H), 5.01-4.75 (m, 1H), 4.21-4.07 (m, 2H), 3.91-3.74 (m, 1H), |
| | | | | 3.71-3.44 (m, 5H), 3.30-3.23 (m, 2H), 3.22-3.11 (m, 2H), 3.09- |
| | | | | 3.00 (m, 1H), 3.00-2.85 (m, 5H), 2.82-2.57 (m, 7H), 2.39-2.20 (m, |
| | | | | 1H), 2.19-2.09 (m, 1H), 1.89-1.65 (m, 2H), 1.52-1.33 (m, 8H), 1.30- |
| | | | | 1.16 (m, 1H), 1.06-0.89 (m, 1H) |
| I-713 | QF | ME | 916.3 | 10.48 (s, 1H), 8.87 (d, J = 4.8 Hz, 1H), 7.95 (s, 1H), 7.73-7.64 (m, 1H), |
| | | | | 7.57 (d, J = 8.0 Hz, 1H), 7.44-7.33 (m, 2H), 7.14 (t, J = 8.0 Hz, 1H), |
| | | | | 6.84 (d, J = 7.6 Hz, 1H), 5.99 (s, 1H), 5.00-4.75 (m, 1H), 4.13 (t, J = |
| | | | | 8.8 Hz, 2H), 3.79-3.67 (m, 1H), 3.67-3.46 (m, 5H), 3.30-3.23 (m, |
| | | | | 2H), 3.17 (t, J = 8.8 Hz, 2H), 3.07-3.00 (m, 1H), 3.00-2.85 (m, 5H), |
| | | | | 2.83-2.58 (m, 7H), 2.40 (s, 3H), 2.37-2.21 (m, 1H), 2.20-2.10 (m, |
| | | | | 1H), 1.85-1.65 (m, 2H), 1.53-1.30 (m, 8H), 1.30-1.16 (m, 1H), 1.05- |
| | | | | 0.89 (m, 1H) |
| I-714 | VT | MM | 902.3 | 10.50 (s, 1H), 8.87 (d, J = 4.8 Hz, 1H), 7.94 (s, 1H), 7.73-7.52 (m, 4H), |
| | | | | 7.38 (dd, J = 8.4, 2.0 Hz, 1H), 7.14 (t, J = 7.6 Hz, 1H), 6.84 (d, J = 7.6 |
| | | | | Hz, 1H), 5.99 (d, J = 2.8 Hz, 1H), 5.01-4.74 (m, 1H), 4.13 (t, J = 8.4 |
| | | | | Hz, 2H), 3.82-3.71 (m, 1H), 3.68-3.46 (m, 5H), 3.30-3.24 (m, 2H), |
| | | | | 3.16 (t, J = 8.4 Hz, 2H), 3.08-2.99 (m, 1H), 2.99-2.84 (m, 5H), 2.83 |
| | | | | 2.59 (m, 7H), 2.38-2.21 (m, 1H), 2.20-2.09 (m, 1H), 1.85-1.67 |
| | | | | (m, 2H), 1.55-1.31 (m, 8H), 1.30-1.15 (m, 1H), 1.07-0.89 (m, 1H) |
| I-715 | VU | MM | 902.4 | 10.50 (s, 1H), 8.87 (d, J = 4.7 Hz, 1H), 8.19 (s, 1H), 7.94 (s, 1H), 7.70- |
| | | | | 7.62 (m, 2H), 7.61-7.52 (m, 2H), 7.38 (dd, J = 8.3, 2.0 Hz, 1H), 7.14 |
| | | | | (t, J = 7.8 Hz, 1H), 6.84 (d, J = 7.5 Hz, 1H), 5.99 (d, J = 2.9 Hz, 1H), |
| | | | | 4.98-4.77 (m, 1H), 4.13 (t, J = 8.6 Hz, 2H), 3.79-3.70 (m, 1H), 3.66- |
| | | | | 3.48 (m, 6H), 3.35-3.27 (m, 1H), 3.17 (t, J = 8.6 Hz, 2H), 3.03 (dt, J = |
| | | | | 10.9, 5.3 Hz, 1H), 2.95 (d, J = 4.9 Hz, 3H), 2.91-2.86 (m, 1H), 2.82-2.63 |
| | | | | (m, 8H), 2.34-2.23 (m, 1H), 2.17-2.10 (m, 1H), 1.84-1.70 (m, 2H), 1.50- |
| | | | | 1.35 (m, 8H), 1.28-1.17 (m, 1H), 1.03-0.92 (m, 1H) |
| I-76 | UK | MM | 833.3 | 10.53 (s, 1H), 8.89 (d, J = 4.7 Hz, 1H), 8.33 (d, J = 2.9 Hz, 1H), 7.96 (s, |
| | | | | 1H), 7.75-7.62 (m, 4H), 7.59-7.54 (m, 1H), 7.43-7.35 (m, 2H), 7.29- |
| | | | | 7.23 (m, 2H), 6.00 (s, 1H), 4.92-4.75 (m, 1H), 4.14 (t, J = 8.4 Hz, 2H), |
| | | | | 3.81-3.72 (m, 1H), 3.66-3.50 (m, 3H), 3.44-3.36 (m, 4H), 3.26-3.17 (m, |
| | | | | 1H), 3.07-3.00 (m, 1H), 2.96 (d, J = 4.9 Hz, 3H), 2.78-2.72 (m, 2H), |
| | | | | 2.57-2.53 (m, 2H), 1.96-1.83 (m, 2H), 1.74-1.53 (m, 4H), 1.26-1.18 (m, |
| | | | | 1H), 1.03-0.91 (m, 1H) |
| I-782 | PR | SZ | 813.4 | 10.58-10.49 (m, 1H), 8.91-8.85 (m, 1H), 8.61-8.56 (m, 1H), 8.48 (s, |
| | | | | 1H), 7.96 (s, 1H), 7.80-7.67 (m, 4H), 7.51 (s, 1H), 7.32-7.25 (m, 2H), |
| | | | | 6.02-5.97 (m, 1H), 4.99-4.76 (m, 1H), 4.18-4.09 (m, 2H), 3.86-3.66 (m, |
| | | | | 4H), 3.63-3.54 (m, 3H), 3.49-3.39 (m, 4H), 3.11-3.04 (m, 4H), 2.97 (dd, |
| | | | | J = 5.0, 2.0 Hz, 3H), 2.79-2.71 (m, 2H), 2.22 (s, 3H), 1.81-1.67 (m, 4H), |
| | | | | 1.27-1.18 (m, 1H), 1.03-0.92 (m, 1H) |
| I-819 | SA | ME | 908.4 | 10.50 (s, 1H), 9.12-9.01 (m, 1H), 8.60 (s, 1H), 7.96-7.66 (m, 5H), 7.46- |
| | | | | 7.28 (m, 4H), 6.02-5.93 (m, 1H), 4.42-4.30 (m, 1H), 4.26-4.03 (m, 4H), |
| | | | | 3.95-3.84 (m, 1H), 3.81-3.61 (m, 7H), 3.50-3.44 (m, 3H), 3.24-3.16 (m, |
| | | | | 5H), 3.00-2.94 (m, 3H), 2.81-2.71 (m, 2H), 2.42 (s, 3H), 2.16-2.00 (m, |
| | | | | 4H), 1.61-1.36 (m, 2H) |
| I-829 | TS | MM | 888.4 | 10.49 (s, 1H), 8.88 (d, J = 4.7 Hz, 1H), 7.95 (s, 1H), 7.74-7.47 (m, 5H), |
| | | | | 7.21-7.09 (m, 1H), 6.90-6.80 (m, 1H), 6.01 (s, 1H), 5.02-4.75 (m, 1H), |
| | | | | 4.18-4.09 (m, 2H), 3.82-3.70 (m, 1H), 3.66-3.59 (m, 1H), 3.57-3.44 (m, |
| | | | | 4H), 3.28-3.11 (m, 4H), 3.09-3.01 (m, 1H), 2.96 (d, J = 4.9 Hz, 3H), |
| | | | | 2.93-2.83 (m, 2H), 2.81-2.64 (m, 6H), 2.43-2.07 (m, 3H), 1.85-1.66 (m, |
| | | | | 4H), 1.60-1.50 (m, 2H), 1.47-1.40 (m, 2H), 1.24-1.14 (m, 1H), 1.05- |
| | | | | 0.90 (m, 1H) |
| I-830 | SV | ME | 937.4 | 10.47 (s, 1H), 9.05-8.99 (m, 2H), 8.75 (s, 1H), 7.97-7.92 (m, 2H), 7.72- |
| | | | | 7.66 (m, 1H), 7.46-7.34 (m, 4H), 5.99-5.94 (m, 1H), 4.41-4.31 (m, 1H), |
| | | | | 4.26-4.15 (m, 3H), 3.86 (s, 2H), 3.83-3.70 (m, 2H), 3.66-3.57 (m, 1H), |
| | | | | 3.56-3.43 (m, 3H), 3.21 (s, 3H), 3.19-3.00 (m, 2H), 2.97 (d, J = 4.9 Hz, |
| | | | | 3H), 2.91-2.82 (m, 2H), 2.78-2.72 (m, 2H), 2.66-2.59 (m, 2H), 2.41 (s, |
| | | | | 3H), 2.16-1.98 (m, 2H), 1.93-1.63 (m, 6H), 1.56-1.41 (m, 2H) |
| I-838 | SV | NH | 921.4 | 10.38 (s, 1H), 9.05-8.95 (m, 2H), 8.78-8.72 (m, 1H), 7.97-7.91 (m, 2H), |
| | | | | 7.71-7.66 (m, 1H), 7.46-7.33 (m, 3H), 7.26 (d, J = 10.2 Hz, 1H), 6.01- |
| | | | | 5.92 (m, 1H), 4.41-4.30 (m, 1H), 4.29-4.16 (m, 3H), 3.86 (s, 2H), 3.82- |
| | | | | 3.74 (m, 2H), 3.56-3.47 (m, 3H), 3.29-3.25 (m, 1H), 3.21 (s, 3H), 3.16- |
| | | | | 3.02 (m, 1H), 3.00-2.94 (m, 3H), 2.91-2.54 (m, 7H), 2.22 (s, 3H), 2.16- |
| | | | | 1.97 (m, 2H), 1.90-1.40 (m, 8H) |
| I-85 | PQ | MM | 846.4 | 10.51 (s, 1H), 8.89 (d, J = 4.8 Hz, 1H), 8.61 (d, J = 2.0 Hz, 1H), 8.27 (s, |
| | | | | 2H) (FA), 7.96 (s, 1H), 7.81 (dd, J = 8.0, 2.0 Hz, 1H), 7.78-7.67 (m, |
| | | | | 3H), 7.64 (d, J = 8.4 Hz, 1H), 7.55 (d, J = 2.0 Hz, 1H), 7.39 (dd, J = 8.4, |
| | | | | 2.0 Hz, 1H), 7.35-7.25 (m, 2H), 6.01 (s, 1H), 5.02-4.75 (m, 1H), 4.14 |
| | | | | (t, J = 8.4 Hz, 2H), 3.85-3.57 (m, 5H), 3.53-3.39 (m, 3H), 3.35-3.22 |
| | | | | (m, 2H), 3.12-3.01 (m, 1H), 2.97 (d, J = 4.8 Hz, 3H), 2.82-2.71 (m, |
| | | | | 2H), 2.59 (t, J = 6.8 Hz, 2H), 2.47-2.39 (m, 2H), 1.72-1.42 (m, 6H), |
| | | | | 1.33-1.15 (m, 1H), 1.08-0.91 (m, 1H) |
| I-853 | QF | NH | 900.5 | 10.45 (s, 1H), 8.87 (d, J = 4.8 Hz, 1H), 8.34 (s, 2H) (FA), 7.95 (s, 1H), |
| | | | | 7.71-7.64 (m, 1H), 7.57 (d, J = 8.0 Hz, 1H), 7.34 (d, J = 6.4 Hz, 1H), |
| | | | | 7.24 (d, J = 10.0 Hz, 1H), 7.14 (t, J = 7.6 Hz, 1H), 6.84 (d, J = 7.6 Hz, |
| | | | | 1H), 6.00 (s, 1H), 5.02-4.75 (m, 1H), 4.14 (t, J = 8.4 Hz, 2H), 3.85- |
| | | | | 3.72 (m, 2H), 3.67-3.45 (m, 5H), 3.27-3.12 (m, 4H), 3.10-3.01 (m, |
| | | | | 1H), 2.99-2.85 (m, 4H), 2.84-2.62 (m, 7H), 2.39-2.08 (m, 5H), 1.86- |
| | | | | 1.67 (m, 2H), 1.55-1.31 (m, 8H), 1.29-1.16 (m, 1H), 1.06-0.90 |
| | | | | (m, 1H) |
| I-861 | QF | PW | 898.4 | 10.32 (s, 1H), 8.88 (d, J = 4.7 Hz, 1H), 7.95 (s, 1H), 7.71-7.63 (m, 1H), |
| | | | | 7.58 (d, J = 8.0 Hz, 1H), 7.37 (dd, J = 8.5, 2.2 Hz, 1H), 7.32 (d, J = 2.2 |
| | | | | Hz, 1H), 7.19-7.10 (m, 2H), 6.85 (d, J = 7.6 Hz, 1H), 6.00 (s, 1H), 5.00- |
| | | | | 4.77 (m, 1H), 4.14 (t, J = 8.6 Hz, 2H), 3.84 (s, 3H), 3.60 (t, J = 6.7 Hz, |
| | | | | 2H), 3.56-3.49 (m, 3H), 3.17 (t, J = 8.5 Hz, 2H), 3.06-3.02 (m, 1H), |
| | | | | 2.99-2.93 (m, 4H), 2.92-2.85 (m, 2H), 2.76-2.62 (m, 7H), 2.37-2.22 (m, |
| | | | | 2H), 2.20-2.06 (m, 2H), 1.83-1.71 (m, 2H), 1.50-1.37 (m, 8H), 1.23- |
| | | | | 1.16 (m, 1H), 1.02-0.92 (m, 1H) |
| I-874 | SX | MM | 897.3 | 10.45 (s, 1H), 8.90-8.84 (m, 1H), 8.07 (d, J = 8.8 Hz, 1H), 7.97 (s, 1H), |
| | | | | 7.89-7.83 (m, 1H), 7.78 (d, J = 8.8 Hz, 1H), 7.74-7.68 (m, 1H), 7.65 (d, |
| | | | | J = 8.2 Hz, 1H), 7.62-7.57 (m, 1H), 7.45-7.39 (m, 1H), 7.39-7.32 (m, |
| | | | | 2H), 6.01 (s, 1H), 5.00-4.76 (m, 1H), 4.26-4.13 (m, 3H), 4.00 (s, 2H), |
| | | | | 3.82-3.74 (m, 1H), 3.68-3.59 (m, 1H), 3.53-3.43 (m, 3H), 3.29-3.21 (m, |
| | | | | 1H), 3.16-3.02 (m, 2H), 2.97 (d, J = 4.9 Hz, 3H), 2.90-2.81 (m, 2H), |
| | | | | 2.78-2.72 (m, 2H), 2.64-2.58 (m, 2H), 1.93-1.81 (m, 2H), 1.77-1.57 (m, |
| | | | | 4H), 1.29-1.17 (m, 1H), 1.05-0.92 (m, 1H) |
| I-879 | MO | MM | 928.5 | 10.51 (s, 1H), 9.02 (d, J = 8.8 Hz, 1H), 7.91 (s, 1H), 7.76-7.58 (m, 3H), |
| | | | | 7.55 (d, J = 2.0 Hz, 1H), 7.38 (dd, J = 8.4, 2.0 Hz, 1H), 7.16 (t, J = 7.6 |
| | | | | Hz, 1H), 6.86 (d, J = 7.6 Hz, 1H), 5.93 (s, 1H), 4.41-4.27 (m, 1H), 4.19 |
| | | | | (t, J = 8.4 Hz, 2H), 3.82-3.71 (m, 2H), 3.68-3.45 (m, 6H), 3.33-3.13 |
| | | | | (m, 6H), 3.02-2.84 (m, 5H), 2.84-2.58 (m, 7H), 2.38-2.21 (m, 1H), |
| | | | | 2.19-1.99 (m, 3H), 1.86-1.66 (m, 2H), 1.61-1.32 (m, 10H) |
| I-883 | VH | VK | 882.3 | 10.53 (s, 1H), 8.90 (d, J = 6.4 Hz, 1H), 8.60 (d, J = 2.8 Hz, 1H), 7.98- |
| | | | | 7.89 (m, 2H), 7.85-7.65 (m, 4H), 7.57 (s, 1H), 7.46-7.26 (m, 3H), |
| | | | | 5.99-5.88 (m, 1H), 5.05-4.78 (m, 1H), 4.25-4.03 (m, 3H), 3.97- |
| | | | | 3.56 (m, 7H), 3.52-3.38 (m, 3H), 3.31-3.26 (m, 2H), 3.22-3.14 (m, |
| | | | | 2H), 2.96 (d, J = 4.8 Hz, 3H), 2.90-2.71 (m, 4H), 2.27-2.10 (m, 2H), |
| | | | | 2.10-1.98 (m, 2H) |
| I-901 | PV | MM | 896.3 | 10.50 (s, 1H), 8.88 (d, J = 4.8 Hz, 1H), 8.60 (d, J = 2.0 Hz, 1H), 7.96 (s, |
| | | | | 1H), 7.90-7.71 (m, 3H), 7.71-7.56 (m, 3H), 7.41 (dd, J = 8.4, 2.0 Hz, |
| | | | | 1H), 7.39-7.20 (m, 2H), 6.02 (s, 1H), 5.04-4.68 (m, 1H), 4.22-4.12 |
| | | | | (m, 3H), 3.86-3.58 (m, 4H), 3.52-3.42 (m, 4H), 3.11-3.02 (m, 2H), |
| | | | | 2.97 (d, J = 4.8 Hz, 3H), 2.83-2.64 (m, 4H), 2.58-2.53 (m, 2H), 1.91- |
| | | | | 1.80 (m, 2H), 1.79-1.54 (m, 4H), 1.31-1.15 (m, 1H), 1.07-0.89 |
| | | | | (m, 1H) |
| I-91 | QW | QV | 859.4 | 10.92 (s, 1H), 8.89 (d, J = 4.8 Hz, 1H), 8.58 (d, J = 2.0 Hz, 1H), 7.96 (s, |
| | | | | 1H), 7.85-7.64 (m, 4H), 7.53 (d, J = 8.0 Hz, 1H), 7.41-7.22 (m, 4H), |
| | | | | 6.01 (s, 1H), 4.99-4.73 (m, 1H), 4.34-4.24 (m, 1H), 4.15 (t, J = 8.4 |
| | | | | Hz, 2H), 3.68-3.52 (m, 3H), 3.44 (t, J = 8.4 Hz, 2H), 3.29-3.19 (m, |
| | | | | 1H), 3.10-3.01 (m, 1H), 2.97 (d, J = 4.8 Hz, 3H), 2.86-2.71 (m, 1H), |
| | | | | 2.62-2.52 (m, 1H), 2.47-2.26 (m, 5H), 2.06-1.95 (m, 1H), 1.60- |
| | | | | 1.35 (m, 8H), 1.31-1.16 (m, 3H), 1.05-0.90 (m, 1H) |
| I-1106 | TW | ME | 954.4 | 10.48 (s, 1H), 9.03 (d, J = 8.8 Hz, 1H), 8.52-8.47 (m, 1H), 7.95-7.88 (m, |
| | | | | 2H), 7.76 (dd, J = 11.2, 1.7 Hz, 1H), 7.72-7.65 (m, 1H), 7.43-7.39 (m, |
| | | | | 2H), 7.33 (t, J = 7.9 Hz, 1H), 7.15 (dd, J = 7.7, 2.0 Hz, 1H), 5.98 (s, 1H), |
| | | | | 4.42-4.29 (m, 1H), 4.25-4.16 (m, 3H), 3.83-3.70 (m, 4H), 3.66-3.56 (m, |
| | | | | 1H), 3.52-3.45 (m, 1H), 3.27-3.22 (m, 3H), 3.21 (s, 3H), 3.16-3.04 (m, |
| | | | | 1H), 2.97 (d, J = 4.9 Hz, 3H), 2.83-2.70 (m, 4H), 2.60-2.55 (m, 2H), |
| | | | | 2.41 (s, 3H), 2.17-1.99 (m, 2H), 1.93-1.80 (m, 2H), 1.74-1.66 (m, 2H), |
| | | | | 1.60-1.37 (m, 3H), 1.26-1.21 (m, 1H) |
| I-1082 | SY | MM | 882.3 | 10.54 (s, 1H), 8.95-8.87 (m, 1H), 8.65-8.43 (m, 1H), 7.96 (s, 1H), 7.84- |
| | | | | 7.64 (m, 6H), 7.59-7.50 (m, 1H), 7.37-7.25 (m, 2H), 6.01 (s, 1H), 5.00- |
| | | | | 4.76 (m, 1H), 4.19-4.08 (m, 2H), 3.82-3.56 (m, 7H), 3.55-3.50 (m, 2H), |
| | | | | 3.09-3.00 (m, 1H), 2.96 (d, J = 4.8 Hz, 3H), 2.81-2.67 (m, 4H), 2.64- |
| | | | | 2.54 (m, 2H), 2.47-2.39 (m, 1H), 2.20-2.10 (m, 1H), 1.92-1.80 (m, 2H), |
| | | | | 1.75-1.67 (m, 1H), 1.28-1.17 (m, 1H), 1.04-0.91 (m, 1H) |
| I-1059 | SA | SZ | 875.4 | 10.57 (s, 1H), 9.06 (d, J = 8.8 Hz, 1H), 8.63-8.51 (m, 2H), 7.95-7.86 (m, |
| | | | | 2H), 7.84-7.79 (m, 1H), 7.76-7.68 (m, 2H), 7.65-7.57 (m, 1H), 7.36- |
| | | | | 7.30 (m, 2H), 5.97 (s, 1H), 4.38-4.28 (m, 6H), 4.23-4.18 (m, 2H), 4.00- |
| | | | | 3.90 (m, 2H), 3.80-3.76 (m, 1H), 3.70 (s, 3H), 3.57-3.52 (m, 1H), 3.50- |
| | | | | 3.45 (m, 2H), 3.21 (s, 3H), 3.19-3.16 (m, 2H), 2.97 (d, J = 4.9 Hz, 3H), |
| | | | | 2.79-2.72 (m, 2H), 2.24 (s, 3H), 2.15-2.09 (m, 1H), 2.06-2.03 (m, 2H), |
| | | | | 1.57-1.43 (m, 2H) |
| I-1056 | TW | MS | 954.4 | 10.45 (s, 1H), 9.03 (d, J = 8.7 Hz, 1H), 8.52-8.46 (m, 1H), 7.95-7.87 (m, |
| | | | | 2H), 7.75 (dd, J = 11.1, 1.7 Hz, 1H), 7.72-7.65 (m, 1H), 7.47 (d, J = 1.6 |
| | | | | Hz, 1H), 7.40 (d, J = 1.6 Hz, 1H), 7.33 (t, J = 7.9 Hz, 1H), 7.18-7.11 (m, |
| | | | | 1H), 5.97 (s, 1H), 4.42-4.29 (m, 1H), 4.24-4.14 (m, 3H), 3.89-3.77 (m, |
| | | | | 2H), 3.75 (s, 2H), 3.59-3.52 (m, 1H), 3.51-3.42 (m, 1H), 3.24 (t, J = 8.4 |
| | | | | Hz, 2H), 3.21 (s, 3H), 3.17-3.04 (m, 1H), 2.97 (d, J = 4.8 Hz, 3H), 2.88- |
| | | | | 2.63 (m, 4H), 2.62-2.52 (m, 3H), 2.24 (s, 3H), 2.17-1.99 (m, 2H), 1.92- |
| | | | | 1.78 (m, 2H), 1.75-1.63 (m, 3H), 1.58-1.35 (m, 3H) |

^{a}The reaction was run at rt for 1 to 2 hrs. Purification was run under a variety of typical methods including prep-HPLC, reverse phase chromatography, and silica gel chromatography.

### Example 3 (Method 2): Synthesis of (S)-2-(5-(1-(isoquinolin-4-yl)-N-(2-(2-(2-(6-((4-((2-methoxy-3-(pyrimidin-2-yl)phenyl)amino)-5-(methylcarbamoyl)pyridin-2-yl)amino)nicotinamido)ethoxy)ethoxy)ethyl)piperidine-3-carboxamido)-2-oxopyridin-1(2H)-yl)acetic acid (I-915)

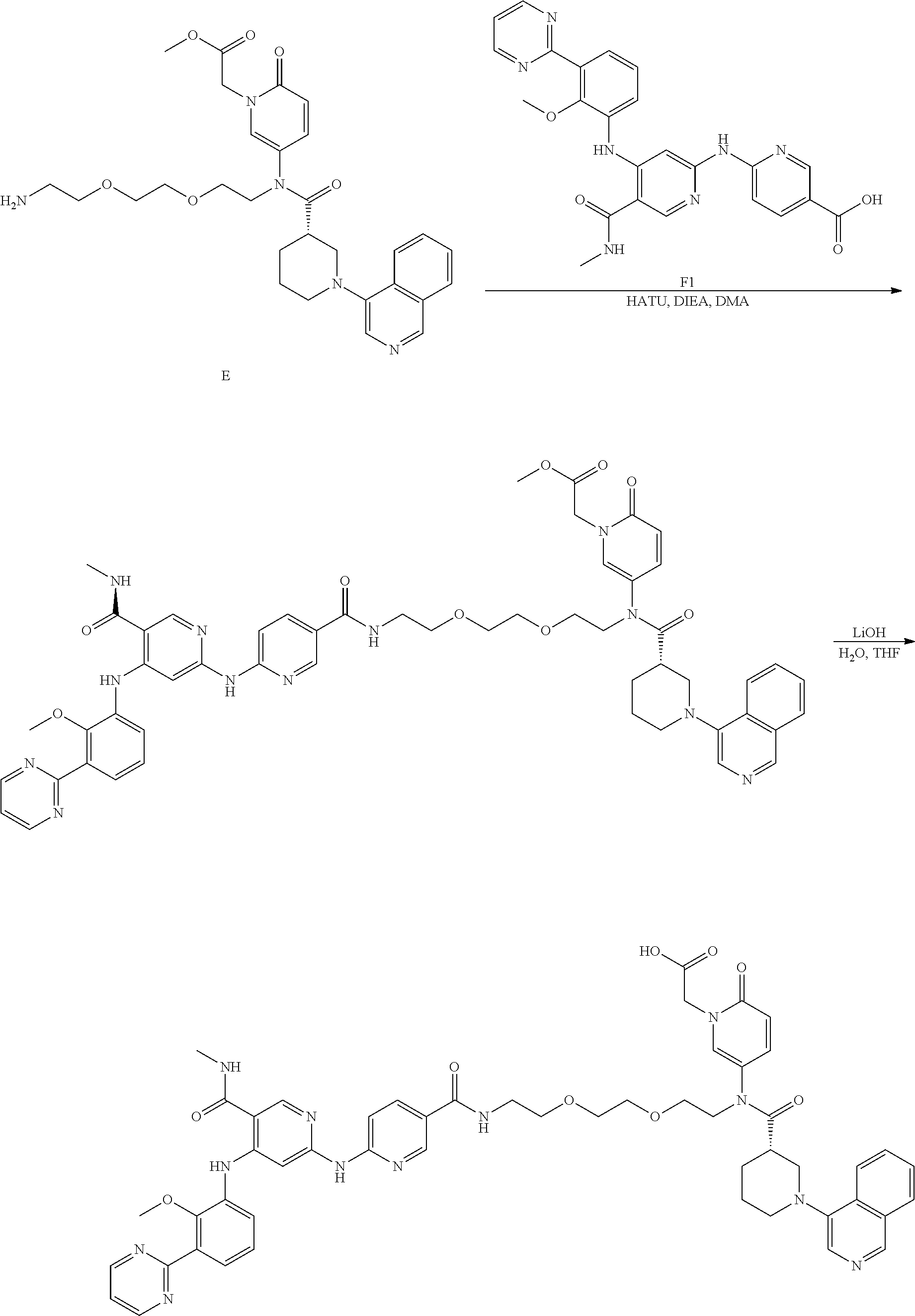

Step 1—Methyl (5)-2-(5-(1-(isoquinolin-4-yl)-N-(2-(2-(2-(6-((4-((2-methoxy-3-(pyrimidin-2-yl)phenyl)amino)-5-(methylcarbamoyl)pyridin-2-yl)amino)nicotinamido)ethoxy)ethoxy)ethyl)piperidine-3-carboxamido)-2-oxopyridin-1(2H)-yl)acetate. To a stirred mixture of 6-((4-((2-methoxy-3-(pyrimidin-2-yl)phenyl)amino)-5-(methylcarbamoyl)pyridin-2-yl)amino)nicotinic acid (85 mg, 0.181 mmol, Intermediate F1) in DMA (4 mL) were added HATU (103 mg, 0.271 mmol) and DIEA (70 mg, 0.543 mmol). The resulting mixture was stirred for 10 min at rt. Next, (S)-2-(5-(N-(2-(2-(2-aminoethoxy)ethoxy)ethyl)-1-(isoquinolin-4-yl)piperidine-3-carboxamido)-2-oxopyridin-1(2H)-yl)acetate (100 mg, 0.181 mmol, Intermediate E) was added to the reaction mixture at rt. Then the resulting mixture was stirred for 1 h at rt. The residue was purified directly without any workup by reverse phase flash chromatography (Column: WelFlash™ C18-I, 20-40 μm, 120 g; Eluent A: Water (plus 10 mmol/L NH₄HCO₃); Eluent B: MeCN; Gradient: 25%-55% B in 25 min; Flow rate: 80 mL/min; Detector: 220 nm; desired fractions were collected at 43% B) and concentrated under reduced pressure to afford the title compound (100 mg, 55% yield) as a yellow solid. LC/MS (ESI, m/z): [(M+H)]⁺=1005.1.

Step 2—(S)-2-(5-(1-(isoquinolin-4-yl)-N-(2-(2-(2-(6-((4-((2-methoxy-3-(pyrimidin-2-yl)phenyl)amino)-5-(methylcarbamoyl)pyridin-2-yl)amino)nicotinamido)ethoxy)ethoxy)ethyl)piperidine-3-carboxamido)-2-oxopyridin-1(2H)-yl)acetic acid. To a stirred mixture of methyl (5)-2-(5-(1-(isoquinolin-4-yl)-N-(2-(2-(2-(6-((4-((2-methoxy-3-(pyrimidin-2-yl)phenyl)amino)-5-(methylcarbamoyl)pyridin-2-yl)amino)nicotinamido)ethoxy)ethoxy)ethyl)piperidine-3-carboxamido)-2-oxopyridin-1(2H)-yl)acetate (100 mg, 0.099 mmol) in H₂O (3 mL) and THF (3 mL) was added LiOH (10 mg, 0.4 mmol) at rt. The resulting mixture was stirred for 1 h at rt. On completion, the mixture was acidified to pH 4 with 4 N HCl (aq). The resulting mixture was concentrated under reduced pressure. The residue was purified by reverse phase flash chromatography (Column: WelFlash™ C18-I, 20-40 μm, 120 g; Eluent A: Water (plus 10 mmol/L NH₄HCO₃); Eluent B: MeCN; Gradient: 25%-35% B in 25 min; Flow rate: 60 mL/min; Detector: 220 nm; desired fractions were collected at 30% B) and concentrated under reduced pressure to afford the title compound (11.2 mg, 11% yield) as a white solid. LC/MS (ESI, m/z): [(M+H)]⁺=991.1. ¹H NMR (400 MHz, DMSO-d₆) δ 10.71 (s, 1H), 10.09 (s, 1H), 9.01-8.92 (m, 3H), 8.70 (d, J=2.4 Hz, 1H), 8.66-8.49 (m, 3H), 8.15-8.07 (m, 2H), 8.05 (d, J=8.0 Hz, 1H), 7.97 (s, 1H), 7.86-7.59 (m, 6H), 7.55-7.47 (m, 2H), 7.43 (dd, J=7.6, 1.6 Hz, 1H), 7.34 (t, J=8.0 Hz, 1l), 6.47 (d, J=9.6 Hz, 1H), 4.72-4.39 (m, 2H), 3.71 (s, 3H), 3.43-3.15 (m, 14H), 2.99-2.85 (m, 1H), 2.86-2.71 (m, 5H), 1.92-1.84 (m, 1H), 1.81-1.51 (m, 3H).

TABLE 3
Compounds synthesized via Method 1, coupling the corresponding amines
and carboxylic acids in Step 1, followed by hydrolysis in Step 2.

| | | | LCMS | |
|---|---|---|---|---|
| | | | (ESI+) | |
| | | | m/z | |
| I-#^{a} | Amine | Acid | (M + H)⁺ | ¹H NMR (400 MHz, DMSO-d₆) δ |
| I- | O | F1 | 1029.2 | 10.71 (s, 1H), 10.08 (s, 1H), 9.02-8.90 (m, 3H), 8.66 (s, 1H), 8.61- |
| 916 | | | | 8.47 (m, 2H), 8.37 (t, J = 5.6 Hz, 1H), 8.13 (s, 1H), 8.10-8.02 (m, |
| | | | | 2H), 7.98 (s, 1H), 7.88 (d, J = 2.8 Hz, 1H), 7.85-7.60 (m, 5H), |
| | | | | 7.56-7.49 (m, 2H), 7.43 (dd, J = 7.6, 1.6 Hz, 1H), 7.33 (t, J = 8.0 |
| | | | | Hz, 1H), 6.52 (d, J = 9.6 Hz, 1H), 4.82-4.47 (m, 2H), 3.71 (s, 3H), |
| | | | | 3.42-3.12 (m, 7H), 2.95-2.85 (m, 1H), 2.85-2.70 (m, 4H), 1.94- |
| | | | | 1.84 (m, 1H), 1.82-1.73 (m, 1H), 1.73-1.56 (m, 2H), 1.55-1.46 (m, |
| | | | | 2H), 1.44-1.36 (m, 2H), 1.34-1.13 (m, 14H) |
| I- | L | F1 | 987.1 | 10.71 (s, 1H), 10.08 (s, 1H), 9.02-8.90 (m, 3H), 8.66 (d, J = 2.4 |
| 917 | | | | Hz, 1H), 8.60-8.49 (m, 2H), 8.49-8.35 (m, 1H), 8.12 (s, 1H), 8.11- |
| | | | | 8.03 (m, 2H), 7.96 (s, 1H), 7.85 (s, 1H), 7.82-7.60 (m, 5H), 7.55- |
| | | | | 7.47 (m, 2H), 7.44 (dd, J = 7.6, 1.6 Hz, 1H), 7.38-7.29 (m, 1H), |
| | | | | 6.50 (d, J = 9.6 Hz, 1H), 4.77-4.44 (m, 2H), 3.71 (s, 3H), 3.40-3.15 |
| | | | | (m, 5H), 2.99-2.85 (m, 1H), 2.86-2.70 (m, 5H), 1.96-1.84 (m, 1H), |
| | | | | 1.82-1.73 (m, 1H), 1.73-1.55 (m, 2H), 1.5-1.47 (m, 2H), 1.46-1.35 |
| | | | | (m, 2H), 1.35-1.19 (m, 9H) |
| I- | G | F1 | 1035.1 | 10.71 (s, 1H), 10.09 (s, 1H), 9.01-8.92 (m, 3H), 8.68 (d, J = 2.4 |
| 918 | | | | Hz, 1H), 8.62-8.48 (m, 3H), 8.16-8.02 (m, 3H), 7.97 (d, J = 2.4 |
| | | | | Hz, 1H), 7.84 (s, 1H), 7.81-7.59 (m, 5H), 7.56-7.49 (m, 2H), 7.43 |
| | | | | (dd, J = 7.6, 1.6 Hz, 1H), 7.33 (t, J = 8.0 Hz, 1H), 6.48 (d, J = 9.6 |
| | | | | Hz, 1H), 4.82-4.28 (m, 2H), 3.71-3.55 (m, 4H), 3.51-3.34 (m, |
| | | | | 15H), 3.33-3.16 (m, 2H), 3.01-2.88 (m, 1H), 2.87-2.73 (m, 5H), |
| | | | | 1.94-1.84 (m, 1H), 1.81-1.50 (m, 3H) |
| I- | R | F1 | 1071.5 | 10.71 (s, 1H), 10.07 (s, 1H), 9.03-8.91 (m, 3H), 8.65 (d, J = 2.4 |
| 919 | | | | Hz, 1H), 8.59-8.50 (m, 2H), 8.36 (t, J = 5.6 Hz, 1H), 8.13 (s, 1H), |
| | | | | 8.11-8.03 (m, 2H), 7.98 (s, 1H), 7.88 (d, J = 2.8 Hz, 1H), 7.83-7.61 |
| | | | | (m, 5H), 7.57-7.48 (m, 2H), 7.43 (dd, J = 7.6, 1.6 Hz, 1H), 7.33 (t, |
| | | | | J = 8.0 Hz, 1H), 6.52 (d, J = 9.6 Hz, 1H), 4.79-4.44 (m, 2H), 3.71 |
| | | | | (s, 3H), 3.30-3.15 (m, 6H), 2.95--2.85 (m, 1H), 2.83-2.76 (m, 5H), |
| | | | | 1.95-1.83 (m, 1H), 1.81-1.74 (m, 1H), 1.72-1.56 (m, 2H), |
| | | | | 1.56-1.46 (m, 2H), 1.44-1.35 (m, 2H), 1.33-1.15 (m, 20H) |
| I- | J | F1 | 1079.4 | 10.70 (s, 1H), 10.07 (s, 1H), 9.01-8.91 (m, 3H), 8.67 (d, J = 2.4 |
| 920 | | | | Hz, 1H), 8.588.43 (m, 3H), 8.13 (s, 1H), 8.18.03 (m, 2H), 7.96 (s, |
| | | | | 1H), 7.97.83 (m, 1H), 7.82-7.60 (m, 5H), 7.58-7.48 (m, 2H), 7.47- |
| | | | | 7.41 (m, 1H), 7.33 (t, J = 7.6 Hz, 1H), 6.54-6.45 (m, 1H), 4.78- |
| | | | | 4.46 (m, 2H), 3.71 (s, 3H), 3.64-3.38 (m, 22H), 2.94-2.72 |
| | | | | (m, 6H), 1.92-1.72 (m, 2H), 1.70-1.54 (m, 2H) |

^{a}The purification of the final compounds was achieved by reverse phase flash chromatography under a variety of conditions.

### Example 4 (Method 2): Synthesis of 6-(4-{[4-({9-[14-chloro-3-(2,4-dioxo-1,3-diazinan-1-yl)benzoyl]-3,9-diazaspiro[5.5]undecan-3-yl}methyl)-3,3-difluoropiperidin-1-yl]methyl}-2,3-dihydroindol-1-yl)-N-[(1R,2R)-2-methoxycyclobutyl]-8-(methylamino)imidazo[1,2-b]pyridazine-3-carboxamide (I-1074)

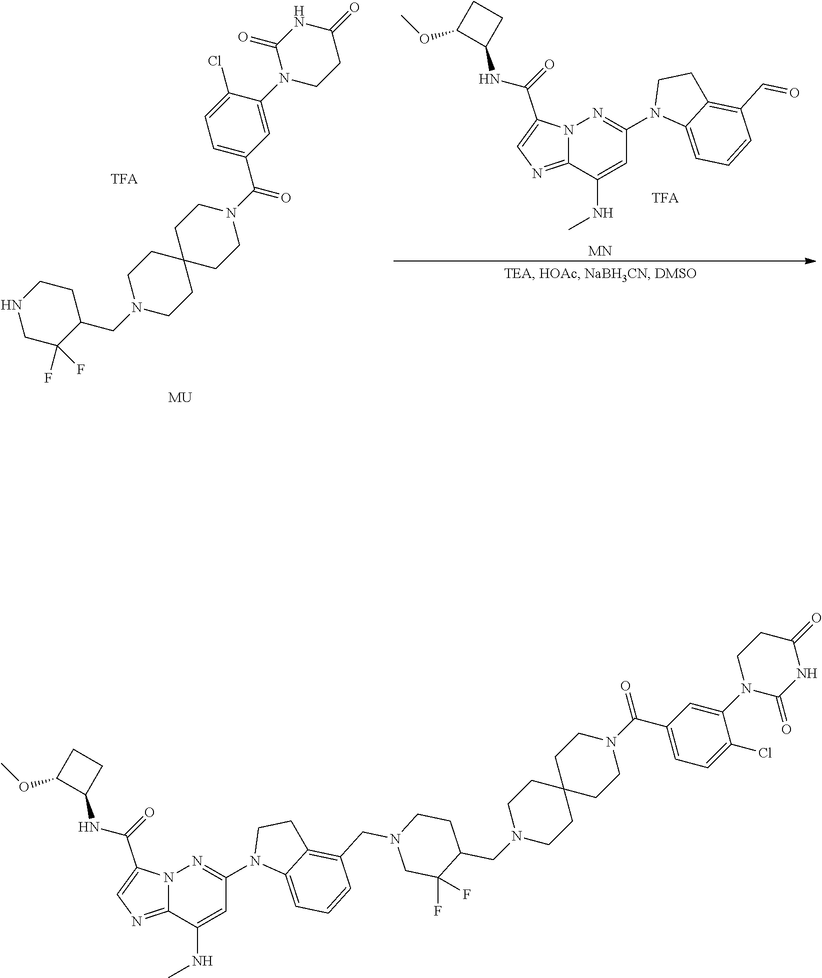

To a stirred mixture of 1-(2-chloro-5-{9-[(3,3-difluoropiperidin-4-yl)methyl]-3,9-diazaspiro[5.5]undecane-3-carbonyl}phenyl)-1,3-diazinane-2,4-dione trifluoroacetate (100 mg, 0.157 mmol, Intermediate MU) and 6-(4-formyl-2,3-dihydroindol-1-yl)-N-[(1R,2R)-2-methoxycyclobutyl]-8-(methylamino)imidazo[1,2-b]pyridazine-3-carboxamide trifluoroacetate (81.52 mg, 0.157 mmol, Intermediate MN) in DMSO (4 mL) was added TEA (0.07 mL, 0.471 mmol) at rt under nitrogen atmosphere. To the above mixture was added AcOH (0.03 mL, 0.471 mmol) at rt. The resulting mixture was stirred for an additional 30 min at 50° C. The mixture was then allowed to cool down to rt. To the above mixture was added NaBH₃CN (19.76 mg, 0.314 mmol) at rt, then the resulting mixture was stirred for additional 16 h at 50° C. On completion, the mixture was cooled to rt and concentrated under vacuum. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 35%-65% B in 25 min; Flow rate: 80 mL/min; Detector: 220/254 nm; desired fractions were collected at 50% B) and concentrated under reduced pressure to afford the title compound (39.3 mg, 26% yield) as a white solid, H NMR (400 MHz, DMSO-d₆) b 10.52 (s, 1H), 9.03 (d, J=8.8 Hz, 1H), 7.90 (s, 1H), 7.73-7.60 (m, 3H), 7.55 (d, J=2.0 Hz, 1H), 7.38 (dd, J=8.2, 2.0 Hz, 1H), 7.17 (t, J=7.8 Hz, 1H), 6.87 (d, J=7.6 Hz, 1H), 5.94 (s, 1H), 4.40-4.27 (m, 1H), 4.19 (t, J=8.5 Hz, 2H), 3.81-3.70 (m, 2H), 3.67-3.46 (m, 5H), 3.31-3.25 (m, 2H), 3.24-3.20 (m, 2H), 3.19 (d, J=1.2 Hz, 3H), 3.00-2.90 (m, 4H), 2.84-2.77 (m, 1H), 2.77-2.70 (m, 2H), 2.48-2.34 (m, 4H), 2.33-2.15 (m, 4H), 2.15-1.98 (m, 41H), 1.93-1.84 (m, 1H), 1.57-1.33 (m, 10H); LC/MS (ESI, m/z): [(M+H)]⁺=942.4.

TABLE 4
Compounds synthesized via Method 2, via the reductive
amination of the corresponding amines and aldehyde.

| | | | LCMS | |
|---|---|---|---|---|
| | | | (ESI+) m/z | |
| I-#^{a} | Amine | Aldehyde | (M + H)⁺ | ¹H NMR (400 MHz, DMSO-d6) |
| I-848 | NF | NG | 860.2 | 10.52 (s, 1H), 10.24 (s, 1H), 9.15 (s, 1H), 8.69-8.62 (m, 2H), |
| | | | | 8.51 (s, 1H), 8.35 (d, J = 1.4 Hz, 1H), 8.20 (d, J = 8.1 Hz, 1H), |
| | | | | 7.95-7.86 (m, 2H), 7.64 (d, J = 8.2 Hz, 1H), 7.55 (d, J = 2.0 Hz, |
| | | | | 1H), 7.39 (dd, J = 8.2, 2.1 Hz, 1H), 4.27 (d, J = 9.7 Hz, 1H), |
| | | | | 4.12-3.98 (m, 3H), 3.96 (s, 3H), 3.81-3.70 (m, 1H), 3.66-3.53 |
| | | | | (m, 5H), 3.31-3.28 (m, 2H), 3.17 (s, 3H), 2.92-2.80 (m, 1H), |
| | | | | 2.78-2.70 (m, 2H), 2.44-2.30 (m, 5H), 2.11 (s, 3H), 1.56-1.35 |
| | | | | (m, 8H) |
| I-341 | IO | NG | 832.2 | 10.51 (s, 1H), 10.23 (s, 1H), 9.15 (s, 1H), 8.67 (d, J = 2.2 Hz, |
| | | | | 1H), 8.65 (s, 1H), 8.50 (s, 1H), 8.36-8.26 (m, 1H), 8.26-8.15 (m, |
| | | | | 1H), 7.95-7.84 (m, 2H), 7.64 (d, J = 8.2 Hz, 1H), 7.55 (d, J = |
| | | | | 2.0 Hz, 1H), 7.42-7.32 (m, 1H), 4.31-4.21 (m, 1H), 4.14-3.98 |
| | | | | (m, 3H), 3.97 (s, 3H), 3.81-3.69 (m, 3H), 3.66-3.49 (m, 3H), |
| | | | | 3.29-3.22 (m, 2H), 3.18 (s, 3H), 3.08-3.02 (m, 4H), 2.93-2.80 |
| | | | | (m, 1H), 2.79-2.70 (m, 2H), 2.59-2.54 (m, 1H), 2.12 |
| | | | | (s, 3H), 1.83-1.64 (m, 4H) |
| I-1113 | NK | NO | 918.2 | 11.04 (s, 1H), 10.22 (s, 1H), 9.16 (s, 1H), 8.70 (d, J = 2.1 Hz, |
| | | | | 1H), 8.65 (d, J = 1.0 Hz, 1H), 8.51 (s, 1H), 8.36 (d, J = 1.5 Hz, |
| | | | | 1H), 8.23 (d, J = 8.1 Hz, 1H), 7.97-7.90 (m, 2H), 6.94 (d, J = |
| | | | | 8.6 Hz, 1H), 6.83 (d, J = 2.2 Hz, 1H), 6.65-6.59 (m, 1H), 5.32- |
| | | | | 5.25 (m, 1H), 4.28 (d, J = 9.7 Hz, 1H), 4.13-3.99 (m, 3H), 3.98 |
| | | | | (s, 3H), 3.73 (s, 2H), 3.31-3.30 (m, 2H), 3.18 (s, 3H), 3.10-3.03 |
| | | | | (m, 4H), 2.99-2.81 (m, 8H), 2.74-2.55 (m, 4H), 2.46-2.30 (m, |
| | | | | 2H), 2.29-2.18 (m, 1H), 2.12 (s, 3H), 2.04-1.95 (m, |
| | | | | 1H), 1.92-1.76 (m, 2H) |
| I-1096 | NR | NP | 901.2 | 10.40 (s, 1H), 9.07 (d, J = 8.8 Hz, 1H), 8.60 (d, J = 2.1 Hz, 1H), |
| | | | | 7.93 (s, 1H), 7.89 (dd, J = 6.7, 2.3 Hz, 1H), 7.82 (dd, J = 8.2, |
| | | | | 2.2 Hz, 1H), 7.78-7.69 (m, 2H), 7.38-7.32 (m, 2H), 7.27 (d, J = |
| | | | | 9.0 Hz, 1H), 6.87 (d, J = 3.0 Hz, 1H), 6.76 (dd, J = 9.1, 3.0 Hz, |
| | | | | 1H), 5.98 (s, 1H), 4.40-4.31 (m, 1H), 4.21 (t, J = 8.5 Hz, 2H), |
| | | | | 3.82-3.75 (m, 1H), 3.73-3.66 (m, 1H), 3.60-3.53 (m, 3H), 3.49 |
| | | | | (t, J = 8.4 Hz, 2H), 3.21 (s, 3H), 3.00-2.87 (m, 7H), 2.78-2.65 |
| | | | | (m, 5H), 2.37-2.24 (m, 4H), 2.15-1.95 (m, 4H), 1.76- |
| | | | | 1.57 (m, 6H), 1.55-1.40 (m, 4H) |
| I-964 | OT | NO | 868.3 | 10.52 (s, 1H), 10.22 (s, 1H), 9.15 (s, 1H), 8.71-8.63 (m, 2H), |
| | | | | 8.50 (s, 1H), 8.36 (s, 1H), 8.22-8.20 (m, 1H), 7.96-7.88 (m, 2H), |
| | | | | 7.82-7.78 (m, 1H), 7.69-7.64 (m, 2H), 4.40-4.35 (m, 1H), 4.31- |
| | | | | 4.26 (m, 1H), 4.21-4.13 (m, 1H), 4.13-4.02 (m, 3H), 3.97 (s, |
| | | | | 3H), 3.87-3.74 (m, 3H), 3.72 (s, 2H), 3.65-3.56 (m, 2H), 3.54- |
| | | | | 3.49 (m, 1H), 3.18 (s, 3H), 2.91-2.70 (m, 5H), 2.57- |
| | | | | 2.53 (m, 1H), 2.12 (s, 3H), 2.06-1.99 (m, 2H) |
| I-929 | PL | NO | 902.4 | 10.84 (s, 1H), 10.23 (s, 1H), 9.16 (s, 1H), 8.71 (d, J = 2.3 Hz, |
| | | | | 1H), 8.65 (d, J = 1.0 Hz, 1H), 8.51 (s, 1H), 8.37 (d, J = 1.4 Hz, |
| | | | | 1H), 8.24 (d, J = 8.1 Hz, 1H), 8.19 (s, 1H), 7.98-7.90 (m, 2H), |
| | | | | 7.50 (d, J = 9.0 Hz, 1H), 6.92 (dd, J = 9.1, 2.0 Hz, 1H), 6.84 (d, |
| | | | | J = 1.9 Hz, 1H), 4.32-4.22 (m, 2H), 4.13-3.99 (m, 3H), 3.98 (s, |
| | | | | 3H), 3.89 (s, 3H), 3.74 (s, 2H), 3.21-3.18 (m, 5H), 3.10-3.00 (m, |
| | | | | 1H), 2.99-2.81 (m, 7H), 2.66-2.53 (m, 4H), 2.45-2.37 (m, 1H), |
| | | | | 2.37-2.13 (m, 4H), 2.12 (s, 3H), 1.93-1.80 (m, 2H) |
| I-922 | PM | NO | 920.4 | 10.85 (s, 1H), 10.23 (s, 1H), 9.16 (s, 1H), 8.71 (d, J = 2.1 Hz, |
| | | | | 1H), 8.65 (s, 1H), 8.51 (s, 1H), 8.37 (d, J = 1.5 Hz, 1H), 8.24 |
| | | | | (d, J = 8.3 Hz, 1H), 7.97-7.90 (m, 2H), 7.46 (d, J = 12.6 Hz, |
| | | | | 1H), 7.11 (d, J = 7.0 Hz, 1H), 4.32-4.23 (m, 2H), 4.13-3.99 (m, |
| | | | | 3H), 3.98 (s, 3H), 3.95 (s, 3H), 3.74 (s, 2H), 3.18 (s, 3H), 3.09- |
| | | | | 3.02 (m, 5H), 3.00-2.81 (m, 8H), 2.70-2.53 (m, 4H), 2.46-2.29 |
| | | | | (m, 3H), 2.29-2.19 (m, 1H), 2.18-2.13 (m, 1H), 1.93- |
| | | | | 1.80 (m, 2H) |
| I-1176b | DX | DT | 1096.7 | (Methanol-d4) 8.96 (d, J = 5.0 Hz, 2H), 8.88 (s, 1H), 8.51 (d, |
| | | | | J = 2.9 Hz, 1H), 8.26 (d, J = 8.8 Hz, 1H), 7.77-7.62 (m, 2H), |
| | | | | 7.56-7.49 (m, 2H), 7.43 (t, J = 7.8 Hz, 1H), 7.21 (td, J = 8.3, |
| | | | | 7.1 Hz, 1H), 7.14 (d, J = 8.8 Hz, 1H), 6.80 (dd, J = 8.2, 2.4 Hz, |
| | | | | 1H), 6.73 (dt, J = 12.9, 2.4 Hz, 1H), 6.62-6.56 (m, 1H), 6.49 |
| | | | | (td, J = 8.3, 2.3 Hz, 1H), 5.86 (d, J = 2.3 Hz, 1H), 4.66-4.57 |
| | | | | (m, 1H), 4.15 (d, J = 12.3 Hz, 3H), 3.78 (t, J = 5.0 Hz, 2H), |
| | | | | 3.74-3.61 (m, 17H), 3.61 (t, J = 5.6 Hz, 2H), 3.57-3.44 (m, 2H), |
| | | | | 3.26 (t, J = 5.0 Hz, 2H), 3.12 (dt, J = 24.1, 10.4 Hz, 5H), 2.98 |
| | | | | (s, 3H), 2.92 (dd, J = 9.4, 3.2 Hz, 1H), 2.48-2.37 (m, 1H), 2.26 |
| | | | | 2.11 (m, 2H), 2.04 (d, J = 3.6 Hz, 1H), 1.96 (s, 6H), 1.90 |
| | | | | (d, J = 14.0 Hz, 3H), 1.71 (s, 4H), 1.52-1.38 (m, |
| | | | | 1H), 1.30 (d, J = 11.0 Hz, 3H) |
| I-1171 | VW | MN | 956.4 | 10.47 (s, 1H), 9.12-8.97 (m, 1H), 7.90 (s, 1H), 7.79-7.60 (m, |
| | | | | 2H), 7.50-7.31 (m, 2H), 7.25-7.09 (m, 1H), 6.95-6.80 (m, 1H), |
| | | | | 5.93 (s, 1H), 4.42-4.28 (m, 1H), 4.18 (s, 2H), 3.86-3.73 (m, 2H), |
| | | | | 3.62-3.49 (m, 5H), 3.24-3.14 (m, 5H), 3.01-2.90 (m, 4H), 2.85- |
| | | | | 2.69 (m, 3H), 2.44-2.37 (m, 2H), 2.30-2.18 (m, 7H), |
| | | | | 2.14-1.86 (m, 6H), 1.60-1.30 (m, 13H) |
| I-1182 | VZ | VY | 972.4 | 10.64 (s, 1H), 9.09 (d, J = 8.8 Hz, 1H), 7.90 (s, 1H), 7.68-7.60 |
| | | | | (m, 2H), 7.41 (d, J = 8.0 Hz, 1H), 7.13 (t, J = 8.0 Hz, 1H), 6.61 |
| | | | | (d, J = 8.1 Hz, 1H), 5.93 (s, 1H), 4.40-4.27 (m, 1H), 4.16 (t, J = |
| | | | | 8.3 Hz, 2H), 3.92-3.71 (m, 2H), 3.69-3.59 (m, 2H), 3.49-3.41 |
| | | | | (m, 2H), 3.30-3.24 (m, 2H), 3.20 (s, 3H), 3.09 (t, J = 8.3 Hz, |
| | | | | 2H), 3.05-3.00 (m, 1H), 2.95 (d, J = 4.9 Hz, 3H), 2.86-2.79 (m, |
| | | | | 1H), 2.75 (t, J = 6.6 Hz, 2H), 2.69-2.60 (m, 5H), 2.58-2.54 (m, |
| | | | | 2H), 2.48-2.42 (m, 1H), 2.40-2.26 (m, 5H), 2.25-2.19 (m, 1H), |
| | | | | 2.15-2.00 (m, 4H), 1.98-1.89 (m, 1H), 1.85-1.74 (m, 2H), 1.71- |
| | | | | 1.61 (m, 1H), 1.57-1.33 (m, 4H), 1.31-1.22 (m, 2H) |
| I-1192 | WR | MJ | 974.4 | 10.46 (s, 1H), 8.85 (d, J = 8.4 Hz, 1H), 7.93 (s, 1H), 7.75-7.64 |
| | | | | (m, 1H), 7.64-7.54 (m, 1H), 7.44-7.35 (m, 2H), 6.67 (d, J = |
| | | | | 9.6 Hz, 1H), 5.93 (s, 1H), 4.40-4.18 (m, 3H), 3.85-3.67 (m, |
| | | | | 2H), 3.67-3.45 (m, 5H), 3.30-3.25 (m, 3H), 3.23-3.10 (m, |
| | | | | 5H), 3.05-2.89 (m, 4H), 2.87-2.69 (m, 3H), 2.47-2.37 (m, |
| | | | | 5H), 2.37-1.83 (m, 9H), 1.59-1.30 (m, 11H) |
| I-1193 | TX | MJ | 974.5 | 10.43 (s, 1H), 8.85 (d, J = 8.4 Hz, 1H), 7.93 (s, 1H), 7.74-7.65 |
| | | | | (m, 1H), 7.59 (dd, J = 10.4, 2.4 Hz, 1H), 7.44 (d, J = 1.6 Hz, |
| | | | | 1H), 7.36 (d, J = 1.6 Hz, 1H), 6.67 (dd, J = 10.0, 2.4 Hz, 1H), |
| | | | | 5.93 (s, 1H), 4.41-4.18 (m, 3H), 3.91-3.70 (m, 2H), 3.66 |
| | | | | 3.47 (m, 5H), 3.23-3.08 (m, 6H), 3.04-2.96 (m, 4H), |
| | | | | 2.90-2.62 (m, 3H), 2.47-2.37 (m, 3H), 2.36-2.16 (m, |
| | | | | 7H), 2.16-1.83 (m, 5H), 1.58-1.32 (m, 12H) |
| I-1200 | WW | MN | 956.5 | 10.51 (s, 1H), 9.02 (d, J = 8.7 Hz, 1H), 7.91 (s, 1H), 7.71-7.64 |
| | | | | (m, 2H), 7.38 (s, 2H), 7.15 (t, J = 7.8 Hz, 1H), 6.86 (d, J = 7.6 |
| | | | | Hz, 1H), 5.91 (s, 1H), 4.40-4.28 (m, 1H), 4.17 (t, J = 8.5 Hz, |
| | | | | 2H), 3.80-3.68 (m, 2H), 3.65-3.46 (m, 5H), 3.38-3.25 (m, 3H), |
| | | | | 3.22-3.17 (m, 4H), 2.99-2.91 (m, 4H), 2.82-2.71 (m, 3H), 2.48- |
| | | | | 2.35 (m, 6H), 2.34-2.17 (m, 4H), 2.14-2.00 (m, 4H), |
| | | | | 1.93-1.83 (m, 1H), 1.58-1.31 (m, 11H) |
| I-1201 | WY | MN | 956.5 | 10.47 (s, 1H), 9.02 (d, J = 8.7 Hz, 1H), 7.91 (s, 1H), 7.73-7.60 |
| | | | | (m, 2H), 7.38 (s, 2H), 7.17 (t, J = 7.8 Hz, 1H), 6.87 (d, J = 7.6 |
| | | | | Hz, 1H), 5.95 (s, 1H), 4.41-4.28 (m, 1H), 4.19 (t, J = 8.4 Hz, |
| | | | | 2H), 3.83-3.69 (m, 2H), 3.65-3.46 (m, 5H), 3.28-3.16 (m, 6H), |
| | | | | 3.02-2.89 (m, 4H), 2.86-2.71 (m, 3H), 2.49-2.32 (m, 7H), |
| | | | | 2.31-2.17 (m, 4H), 2.16-2.00 (m, 4H), 1.96-1.84 (m, |
| | | | | 1H), 1.57-1.35 (m, 11H) |
| I-1 | QY | IN | 882.3 | 11.06 (s, 1H), 8.89 (d, J = 4.7 Hz, 1H), 8.60 (d, J = 2.1 Hz, 1H), |
| | | | | 7.99-7.94 (m, 1H), 7.82 (dd, J = 8.2, 2.2 Hz, 1H), 7.79-7.68 (m, |
| | | | | 3H), 7.36-7.26 (m, 2H), 6.94 (d, J = 8.6 Hz, 1H), 6.83 (d, J = |
| | | | | 2.2 Hz, 1H), 6.62 (dd, J = 8.7, 2.2 Hz, 1H), 6.02 (s, 1H), 5.29 |
| | | | | (dd, J = 13.0, 5.4 Hz, 1H), 4.99-4.78 (m, 1H), 4.16 (t, J = 8.5 |
| | | | | Hz, 2H), 3.55 (s, 2H), 3.45 (t, J = 8.5 Hz, 2H), 3.31 (s, 3H), |
| | | | | 3.12-3.00 (m, 5H), 2.97 (d, J = 4.9 Hz, 3H), 2.94-2.83 (m, 3H), |
| | | | | 2.75-2.62 (m, 6H), 2.30-2.21 (m, 1H), 2.07-1.95 (m, 3H), 1.84- |
| | | | | 1.76 (m, 2H), 1.55-1.42 (m, 2H), 1.24-1.16 (m, 1H), 1.06-0.91 |
| | | | | (m, 1H) |
| I-953 | TX | MN | 956.4 | 10.45 (s, 1H), 9.03 (d, J = 8.8 Hz, 1H), 7.91 (s, 1H), 7.72-7.64 |
| | | | | (m, 2H), 7.45-7.43 (m, 1H), 7.37-7.35 (m, 1H), 7.20-7.15 (m, |
| | | | | 1H), 6.87 (d, J = 7.6 Hz, 1H), 5.94 (s, 1H), 4.40-4.29 (m, 1H), |
| | | | | 4.19 (t, J = 8.5 Hz, 2H), 3.87-3.74 (m, 2H), 3.61-3.49 (m, 5H), |
| | | | | 3.24-3.18 (m, 5H), 3.00-2.92 (m, 4H), 2.87-2.78 (m, 2H), 2.74- |
| | | | | 2.66 (m, 1H), 2.48-2.35 (m, 4H), 2.34-2.27 (m, 2H), 2.26-2.18 |
| | | | | (m, 5H), 2.16-2.01 (m, 4H), 1.94-1.86 (m, 1H), 1.60- |
| | | | | 1.32 (m, 12H) |
| I-19 | QI | QG | 891.5 | 11.08 (s, 1H), 10.16 (s, 1H), 8.74 (d, J = 4.4 Hz, 1H), 7.91 (s, |
| | | | | 1H), 7.77-7.61 (m, 2H), 7.27-7.13 (m, 1H), 7.01-6.84 (m, |
| | | | | 2H), 6.70-6.62 (m, 1H), 5.92 (s, 1H), 5.39-5.23 (m, 1H), 4.18 |
| | | | | (t, J = 8.4 Hz, 2H), 4.10-3.97 (m, 3H), 3.67-3.47 (m, 4H), |
| | | | | 3.33-3.27 (m, 4H), 3.21 (t, J = 8.4 Hz, 2H), 3.16-3.05 (m, |
| | | | | 2H), 3.03-2.84 (m, 7H), 2.75-2.56 (m, 3H), 2.42-2.16 (m, |
| | | | | 2H), 2.07-1.66 (m, 8H), 1.62-1.39 (m, 3H), 0.84-0.74 (m, |
| | | | | 2H), 0.61-0.51 (m, 2H) |
| I-277 | RH | QX | 973.4 | 11.08 (s, 1H), 8.95-8.87 (m, 1H), 8.60 (s, 1H), 7.96 (s, 1H), |
| | | | | 7.85-7.80 (m, 1H), 7.79-7.74 (m, 2H), 7.73-7.68 (m, 1H), 7.35- |
| | | | | 7.27 (m, 2H), 7.02-6.87 (m, 3H), 6.05-5.99 (m, 1H), 5.35 (dd, J = |
| | | | | 12.8, 5.4 Hz, 1H), 4.99-4.76 (m, 1H), 4.15 (t, J = 8.5 Hz, 2H), |
| | | | | 3.68 (s, 2H), 3.62 (s, 3H), 3.52-3.43 (m, 3H), 3.41-3.37 (m, 1H), |
| | | | | 3.05-2.95 (m, 8H), 2.91-2.79 (m, 4H), 2.76-2.68 (m, 2H), 2.64- |
| | | | | 2.57 (m, 3H), 2.39-2.33 (m, 1H), 2.20-2.11 (m, 1H), 2.04-1.97 |
| | | | | (m, 1H), 1.78-1.70 (m, 1H), 1.62-1.52 (m, 1H), 1.29-1.19 (m, |
| | | | | 4H), 1.03-0.93 (m, 1H), 0.88-0.81 (m, 1H) |
| I-279 | UQ | QX | 958.4 | 11.08 (s, 1H), 8.89 (d, J = 4.7 Hz, 1H), 8.62-8.59 (m, 1H), 7.96 |
| | | | | (s, 1H), 7.84-7.80 (m, 1H), 7.78-7.73 (m, 2H), 7.72-7.67 (m, |
| | | | | 1H), 7.34-7.27 (m, 2H), 6.99-6.93 (m, 1H), 6.90-6.85 (m, 2H), |
| | | | | 6.04-5.98 (m, 1H), 5.35 (dd, J = 12.6, 5.4 Hz, 1H), 4.98-4.77 |
| | | | | (m, 1H), 4.14 (t, J = 8.7 Hz, 2H), 3.66 (s, 2H), 3.62 (s, 3H), 3.45 |
| | | | | (t, J = 8.5 Hz, 2H), 3.24-2.99 (m, 6H), 2.97 (d, J = 4.9 Hz, 3H), |
| | | | | 2.95-2.79 (m, 3H), 2.75-2.55 (m, 7H), 2.39-2.31 (m, 1H), 2.04- |
| | | | | 1.96 (m, 1H), 1.94-1.67 (m, 5H), 1.62-1.52 (m, 1H), 1.27-1.19 |
| | | | | (m, 1H), 1.04-0.91 (m, 1H) |
| I-28 | T | QK | 825.4 | 11.06 (s, 1H), 9.05 (d, J = 8.8 Hz, 1H), 8.66 (d, J = 4.8 Hz, 1H), |
| | | | | 7.96-7.84 (m, 2H), 7.77-7.64 (m, 2H), 7.43-7.30 (m, 3H), |
| | | | | 6.95 (d, J = 8.4 Hz, 1H), 6.86 (d, J = 2.4 Hz, 1H), 6.64 (dd, J = |
| | | | | 8.8, 2.4 Hz, 1H), 5.97 (s, 1H), 5.29 (dd, J = 12.8, 5.2 Hz, 1H), |
| | | | | 4.42-4.30 (m, 1H), 4.21 (t, J = 8.4 Hz, 2H), 3.86-3.74 (m, |
| | | | | 1H), 3.69 (s, 2H), 3.55-3.44 (m, 2H), 3.40-3.34 (m, 3H), 3.21 |
| | | | | (s, 3H), 3.19-3.11 (m, 4H), 2.97 (d, J = 4.8 Hz, 3H), 2.94- |
| | | | | 2.82 (m, 1H), 2.77-2.56 (m, 6H), 2.18-1.95 (m, 3H), |
| | | | | 1.60-1.37 (m, 2H) |
| I-282 | Q0 | QX | 973.5 | 11.06 (s, 1H), 8.94-8.83 (m, 1H), 8.65-8.57 (m, 1H), 7.96 (s, |
| | | | | 1H), 7.85-7.79 (m, 1H), 7.78-7.73 (m, 2H), 7.73-7.66 (m, 1H), |
| | | | | 7.37-7.27 (m, 2H), 6.96-6.91 (m, 1H), 6.85-6.80 (m, 1H), 6.64- |
| | | | | 6.58 (m, 1H), 6.01 (s, 1H), 5.29 (dd, J = 12.8, 5.3 Hz, 1H), 4.99- |
| | | | | 4.76 (m, 1H), 4.15 (t, J = 8.6 Hz, 2H), 3.67 (s, 2H), 3.52-3.43 |
| | | | | (m, 3H), 3.41-3.36 (m, 2H), 3.30 (s, 3H), 3.11-3.05 (m, 4H), |
| | | | | 3.00-2.93 (m, 5H), 2.90-2.82 (m, 2H), 2.71-2.56 (m, 5H), 2.43- |
| | | | | 2.34 (m, 6H), 2.03-1.94 (m, 1H), 1.79-1.66 (m, 1H), 1.62-1.51 |
| | | | | (m, 1H), 1.29-1.16 (m, 1H), 1.04-0.92 (m, 1H) |
| I-347 | QZ | KO | 924.4 | 11.27-10.72 (m, 1H), 8.94-8.85 (m, 1H), 7.95 (s, 1H), 7.73-7.66 |
| | | | | (m, 1H), 7.61-7.56 (m, 1H), 7.15 (t, J = 7.8 Hz, 1H), 6.97-6.91 |
| | | | | (m, 1H), 6.87-6.82 (m, 2H), 6.65-6.58 (m, 1H), 6.00 (s, 1H), |
| | | | | 5.29 (dd, J = 12.9, 5.4 Hz, 1H), 4.99-4.78 (m, 1H), 4.14 (t, J = |
| | | | | 8.7 Hz, 2H), 3.57-3.48 (m, 2H), 3.31 (s, 3H), 3.21-3.14 (m, 2H), |
| | | | | 3.08-3.01 (m, 5H), 2.98-2.93 (m, 4H), 2.92-2.86 (m, 2H), 2.67- |
| | | | | 2.59 (m, 5H), 2.55 (s, 3H), 2.37-2.09 (m, 4H), 2.03-1.94 (m, |
| | | | | 1H), 1.87-1.67 (m, 4H), 1.44-1.31 (m, 2H), 1.27-1.21 (m, 2H), |
| | | | | 1.05-0.91 (m, 1H), 0.89-0.81 (m, 1H) |
| I-359b | QU | RO | 972.5 | 11.05 (s, 1H), 8.88 (d, J = 4.8 Hz, 1H), 8.55 (d, J = 2.0 Hz, 1H), |
| | | | | 7.95 (s, 1H), 7.79-7.65 (m, 4H), 7.39-7.21 (m, 2H), 6.87 (d, |
| | | | | J = 8.4 Hz, 1H), 6.77 (d, J = 2.0 Hz, 1H), 6.62-6.53 (m, 1H), |
| | | | | 5.98 (s, 1H), 5.34-5.19 (m, 1H), 5.00-4.73 (m, 1H), 4.14 (t, |
| | | | | J = 8.4 Hz, 2H), 3.62-3.50 (m, 2H), 3.50-3.36 (m, 3H), 3.28 |
| | | | | (s, 3H), 3.11-2.93 (m, 7H), 2.91-2.83 (m, 3H), 2.76-2.54 |
| | | | | (m, 7H), 2.43-2.29 (m, 3H), 2.04-1.95 (m, 1H), 1.79-1.69 |
| | | | | (m, 2H), 1.69-1.59 (m, 1H), 1.58-1.38 (m, 4H), 1.30-1.11 |
| | | | | (m, 1H), 1.07-0.89 (m, 1H) |
| I-361 | UM | KC | 902.4 | 10.50 (s, 1H), 8.88 (d, J = 4.7 Hz, 1H), 7.95 (s, 1H), 7.69-7.64 |
| | | | | (m, 1H), 7.62 (s, 1H), 7.60-7.53 (m, 2H), 7.39 (dd, J = 8.3, 2.0 |
| | | | | Hz, 1H), 7.14 (t, J = 7.7 Hz, 1H), 6.85 (d, J = 7.6 Hz, 1H), 6.00 |
| | | | | (s, 1H), 5.01-4.77 (m, 1H), 4.14 (t, J = 8.5 Hz, 2H), 3.80-3.71 |
| | | | | (m, 1H), 3.65-3.49 (m, 5H), 3.30 (s, 3H), 3.17 (t, J = 8.5 Hz, |
| | | | | 2H), 3.07-3.01 (m, 1H), 2.96 (d, J = 4.9 Hz, 3H), 2.93-2.87 (m, |
| | | | | 1H), 2.79-2.64 (m, 7H), 2.35-2.23 (m, 1H), 2.18-2.10 (m, 1H), |
| | | | | 1.85-1.72 (m, 2H), 1.50-1.35 (m, 8H), 1.29-1.18 (m, |
| | | | | 1H), 1.02-0.93 (m, 1H) |
| I-382 | TI | TH | 832.4 | 11.05 (s, 1H), 10.18 (s, 1H), 9.09 (s, 1H), 8.66 (s, 1H), 8.37 (s, |
| | | | | 1H), 7.58 (s, 1H), 7.13 (s, 1H), 6.92 (d, J = 8.6 Hz, 1H), 6.81 (d, |
| | | | | J = 2.2 Hz, 1H), 6.62 (dd, J = 8.9, 2.2 Hz, 1H), 5.28 (dd, J = |
| | | | | 13.0, 5.3 Hz, 1H), 4.34 (t, J = 7.2 Hz, 2H), 4.21 (d, J = 9.6 Hz, |
| | | | | 1H), 4.10-3.95 (m, 2H), 3.91 (d, J = 9.6 Hz, 1H), 3.60-3.53 (m, |
| | | | | 2H), 3.30 (s, 3H), 3.12 (s, 3H), 2.92-2.73 (m, 4H), 2.71-2.56 (m, |
| | | | | 4H), 2.47-2.41 (m, 1H), 2.39 (s, 3H), 2.15-2.08 (m, 5H), 2.02- |
| | | | | 1.95 (m, 1H), 1.86-1.68 (m, 8H), 1.65-1.55 (m, 1H), |
| | | | | 1.29-1.16 (m, 5H) |
| I-383 | RB | KO | 936.4 | 11.06 (s, 1H), 8.93-8.85 (m, 1H), 8.29 (s, 1H), 7.95 (s, 1H), |
| | | | | 7.72-7.65 (m, 1H), 7.60-7.53 (m, 1H), 7.14 (t, J = 7.8 Hz, 1H), |
| | | | | 6.96-6.91 (m, 1H), 6.88-6.80 (m, 2H), 6.65-6.57 (m, 1H), 6.00 |
| | | | | (s, 1H), 5.29 (dd, J = 12.9, 5.3 Hz, 1H), 5.01-4.75 (m, 1H), 4.14 |
| | | | | (t, J = 8.6 Hz, 2H), 3.53 (s, 3H), 3.31-3.21 (m, 6H), 3.19-3.12 |
| | | | | (m, 4H), 3.07-3.03 (m, 3H), 2.96 (d, J = 4.9 Hz, 3H), 2.93-2.84 |
| | | | | (m, 2H), 2.83-2.67 (m, 3H), 2.67-2.55 (m, 3H), 2.41-2.36 (m, |
| | | | | 4H), 2.32-2.27 (m, 1H), 2.22-2.15 (m, 2H), 2.03-1.95 (m, 1H), |
| | | | | 1.94-1.86 (m, 2H), 1.72-1.60 (m, 1H), 1.53-1.40 (m, 1H), |
| | | | | 1.27-1.19 (m, 1H), 1.02-0.88 (m, 1H) |
| I-399 | QM | IN | 987.4 | 11.04 (s, 1H), 8.89 (d, J = 4.8 Hz, 1H), 8.60 (s, 1H), 7.98-7.95 |
| | | | | (m, 1H), 7.90-7.63 (m, 4H), 7.41-7.24 (m, 2H), 6.93 (d, J = |
| | | | | 8.4 Hz, 1H), 6.82 (s, 1H), 6.61 (d, J = 8.8 Hz, 1H), 6.02 (s, 1H), |
| | | | | 5.39-5.22 (m, 1H), 5.06-4.71 (m, 1H), 4.15 (t, J = 8.4 Hz, |
| | | | | 2H), 3.67 (s, 2H), 3.53-3.35 (m, 6H), 3.12-3.02 (m, 6H), 2.97 |
| | | | | (d, J = 4.8 Hz, 3H), 3.10-3.02 (m, 5H), 2.97 (d, J = 4.8 Hz, 3H), |
| | | | | 2.92-2.83 (m, 2H), 2.75-2.55 (m, 6H), 2.41-2.30 (m, 1H), |
| | | | | 2.05-1.90 (m, 1H), 1.78-1.64 (m, 1H), 1.60-1.46 (m, 1H), |
| | | | | 1.30-1.15 (m, 1H), 1.06-0.89 (m, 1H) |
| I-429c | RE | RC | 962.4 | 11.09 (s, 1H), 8.91-8.83 (m, 1H), 7.94 (s, 1H), 7.73-7.65 (m, |
| | | | | 1H), 7.57-7.50 (m, 1H), 7.14-7.08 (m, 1H), 7.02-6.87 (m, 3H), |
| | | | | 6.72-6.68 (m, 1H), 5.97 (s, 1H), 5.36 (dd, J = 12.6, 5.4 Hz, 1H), |
| | | | | 5.02-4.74 (m, 1H), 4.13 (t, J = 8.7 Hz, 2H), 3.62 (s, 3H), 3.52- |
| | | | | 3.46 (m, 1H), 3.42-3.37 (m, 1H), 3.22 (t, J = 8.5 Hz, 2H), 3.06- |
| | | | | 2.83 (m, 14H), 2.75-2.66 (m, 2H), 2.65-2.54 (m, 6H), 2.44-2.32 |
| | | | | (m, 1H), 2.25-2.10 (m, 2H), 2.08-1.97 (m, 7H), 1.79-1.66 (m, |
| | | | | 1H), 1.60-1.45 (m, 1H), 1.27-1.19 (m, 1H), 1.02-0.89 (m, 1H) |
| I-445 | SG | SD | 803.3 | 10.90 (s, 1H), 8.93-8.88 (m, 1H), 7.95 (s, 1H), 7.94-7.91 (m, |
| | | | | 1H), 7.71-7.62 (m, 2H), 7.57-7.53 (m, 1H), 7.41-7.37 (m, 1H), |
| | | | | 7.32-7.27 (m, 2H), 7.26-7.20 (m, 2H), 6.93-6.89 (m, 1H), 6.00 |
| | | | | (s, 1H), 5.00-4.74 (m, 1H), 4.20 (dd, J = 12.3, 5.1 Hz, 1H), 4.12 |
| | | | | (t, J = 8.5 Hz, 2H), 3.71-3.65 (m, 4H), 3.51-3.45 (m, 2H), 3.40 |
| | | | | (t, J = 8.5 Hz, 3H), 3.08-3.02 (m, 1H), 2.96 (d, J = 4.9 Hz, 3H), |
| | | | | 2.84-2.72 (m, 1H), 2.58-2.53 (m, 1H), 2.40-2.25 (m, 4H), 2.04- |
| | | | | 1.96 (m, 1H), 1.84-1.74 (m, 4H), 1.28-1.18 (m, 1H), |
| | | | | 1.04-0.92 (m, 1H) |
| I-459 | QU | SB | 917.4 | 11.06 (s, 1H), 8.88 (d, J = 4.8 Hz, 1H), 8.54 (s, 1H), 7.96 (s, |
| | | | | 1H), 7.86-7.52 (m, 4H), 7.40-7.22 (m, 2H), 7.15-6.78 (m, |
| | | | | 3H), 6.01 (s, 1H), 5.47-5.18 (m, 1H), 5.12-4.69 (m, 1H), 4.14 |
| | | | | (t, J = 8.4 Hz, 2H), 3.58-3.32 (m, 7H), 3.10-2.94 (m, 7H), |
| | | | | 2.93-2.82 (m, 4H), 2.81-2.54 (m, 9H), 2.41-2.27 (m, 1H), |
| | | | | 2.08-1.95 (m, 1H), 1.76-1.63 (m, 1H), 1.59-1.45 (m, 1H), |
| | | | | 1.33-1.16 (m, 1H), 1.08-0.89 (m, 1H) |
| I-468 | QM | KO | 910.4 | 11.05 (s, 1H), 8.93-8.83 (m, 1H), 7.95 (s, 1H), 7.71-7.56 (m, |
| | | | | 2H), 7.14 (t, J = 7.8 Hz, 1H), 6.97-6.89 (m, 1H), 6.89-6.78 (m, |
| | | | | 2H), 6.65-6.56 (m, 1H), 6.00 (s, 1H), 5.29 (dd, J = 12.8, 5.3 Hz, |
| | | | | 1H), 4.99-4.75 (m, 1H), 4.13 (t, J = 8.6 Hz, 2H), 3.53 (s, 3H), |
| | | | | 3.48-3.41 (m, 2H), 3.39-3.33 (m, 2H), 3.30-3.25 (m, 4H), 3.21- |
| | | | | 3.14 (m, 3H), 3.10-3.01 (m, 6H), 2.96 (d, J = 4.9 Hz, 3H), 2.91- |
| | | | | 2.84 (m, 3H), 2.82-2.57 (m, 6H), 2.35-2.28 (m, 1H), 2.04-1.92 |
| | | | | (m, 1H), 1.73-1.47 (m, 2H), 1.30-1.15 (m, 2H), 1.05- |
| | | | | 0.90 (m, 1H) |
| I-470 | VD | QX | 991.5 | 11.23-10.94 (m, 1H), 8.90 (d, J = 4.7 Hz, 1H), 8.62-8.57 (m, |
| | | | | 1H), 7.99-7.94 (m, 1H), 7.84-7.80 (m, 1H), 7.78-7.75 (m, 2H), |
| | | | | 7.74-7.70 (m, 1H), 7.35-7.28 (m, 2H), 6.88-6.83 (m, 1H), 6.76- |
| | | | | 6.68 (m, 1H), 6.02 (s, 1H), 5.33 (dd, J = 12.9, 5.4 Hz, 1H), 5.00- |
| | | | | 4.76 (m, 1H), 4.15 (t, J = 8.5 Hz, 2H), 3.67 (s, 2H), 3.51-3.45 |
| | | | | (m, 7H), 3.07-3.03 (m, 1H), 3.00-2.92 (m, 10H), 2.90-2.86 (m, |
| | | | | 1H), 2.71-2.63 (m, 3H), 2.44-2.35 (m, 4H), 2.05-1.97 (m, 1H), |
| | | | | 1.77-1.70 (m, 1H), 1.61-1.51 (m, 1H), 1.29-1.22 (m, 4H), 1.04- |
| | | | | 0.93 (m, 1H), 0.88-0.82 (m, 1H) |
| I-471 | RH | KO | 896.4 | 11.08 (s, 1H), 8.88 (d, J = 4.6 Hz, 1H), 8.15 (s, 0.4H), 7.95 (s, |
| | | | | 1H), 7.72-7.63 (m, 1H), 7.57 (d, J = 8.0 Hz, 1H), 7.14 (t, J = 7.8 |
| | | | | Hz, 1H), 7.03-6.96 (m, 1H), 6.96-6.88 (m, 2H), 6.88-6.82 (m, |
| | | | | 1H), 6.00 (s, 1H), 5.35 (dd, J = 12.6, 5.4 Hz, 1H), 5.00-4.76 (m, |
| | | | | 1H), 4.14 (t, J = 8.7 Hz, 2H), 3.62 (s, 3H), 3.56-3.45 (m, 4H), |
| | | | | 3.43-3.34 (m, 2H), 3.17 (t, J = 8.5 Hz, 2H), 3.09-2.93 (m, 9H), |
| | | | | 2.92-2.80 (m, 4H), 2.79-2.55 (m, 6H), 2.37-2.27 (m, 1H), 2.22- |
| | | | | 2.08 (m, 1H), 2.04-1.95 (m, 1H), 1.76-1.66 (m, 1H), 1.60-1.49 |
| | | | | (m, 1H), 1.30-1.16 (m, 1H), 1.05-0.90 (m, 1H) |
| I-910 | UZ | QX | 936.4 | 11.07 (s, 1H), 8.89 (d, J = 4.7 Hz, 1H), 8.61 (s, 1H), 7.96 (d, |
| | | | | J = 1.7 Hz, 1H), 7.86-7.69 (m, 4H), 7.37-7.26 (m, 2H), 6.86 (d, |
| | | | | J = 8.5 Hz, 1H), 6.73 (t, J = 8.2 Hz, 1H), 6.01 (s, 1H), 5.36-5.30 |
| | | | | (m, 1H), 4.99-4.77 (m, 1H), 4.15 (t, J = 8.5 Hz, 2H), 3.68 (s, |
| | | | | 2H), 3.52-3.42 (m, 4H), 3.30 (s, 3H), 3.10-2.92 (m, 14H), |
| | | | | 2.75-2.60 (m, 2H), 2.45-2.30 (m, 1H), 2.23-2.17 (m, 1H), |
| | | | | 2.01-1.97 (m, 1H), 1.89-1.79 (m, 2H), 1.29-1.16 (m, |
| | | | | 1H), 1.04-0.93 (m, 1H) |
| I-48 | UC | QG | 863.4 | 8.79-8.72 (m, 1H), 8.41 (s, 1H), 7.93-7.88 (m, 1H), 7.66-7.60 |
| | | | | (m, 2H), 7.21-7.15 (m, 1H), 6.96-6.91 (m, 1H), 6.88-6.81 (m, |
| | | | | 2H), 6.66-6.61 (m, 1H), 5.91 (s, 1H), 5.28 (dd, J = 12.9, 5.4 Hz, |
| | | | | 1H), 4.16 (t, J = 8.5 Hz, 2H), 3.58-3.49 (m, 4H), 3.31-3.30 (m, |
| | | | | 3H), 3.24-3.15 (m, 3H), 3.09-3.01 (m, 6H), 2.95 (d, J = 4.9 Hz, |
| | | | | 3H), 2.94-2.75 (m, 4H), 2.71-2.57 (m, 3H), 2.40-2.18 (m, 2H), |
| | | | | 2.06-1.93 (m, 1H), 1.83-1.77 (m, 3H), 1.75-1.67 (m, 1H), 1.59- |
| | | | | 1.49 (m, 1H), 0.83-0.76 (m, 2H), 0.59-0.53 (m, 2H) |
| I-596 | QI | UW | 923.5 | 11.03 (s, 1H), 8.92-8.83 (m, 1H), 7.93 (s, 1H), 7.67-7.61 (m, |
| | | | | 1H), 7.49 (d, J = 8.0 Hz, 1H), 7.11-7.06 (m, 1H), 6.95-6.90 (m, |
| | | | | 1H), 6.83-6.76 (m, 2H), 6.63 (d, J = 8.6 Hz, 1H), 5.98 (s, 1H), |
| | | | | 5.28 (dd, J = 12.9, 5.3 Hz, 1H), 4.97-4.76 (m, 1H), 4.17-4.08 |
| | | | | (m, 2H), 3.20-3.10 (m, 3H), 3.09-2.99 (m, 7H), 2.95 (d, J = 4.9 |
| | | | | Hz, 3H), 2.92-2.83 (m, 1H), 2.81-2.53 (m, 13H), 2.37-2.23 (m, |
| | | | | 1H), 2.20-2.12 (m, 1H), 2.03-1.94 (m, 1H), 1.89-1.68 (m, 2H), |
| | | | | 1.60-1.52 (m, 4H), 1.49-1.41 (m, 4H), 1.25-1.19 (m, |
| | | | | 1H), 1.02-0.90 (m, 1H) |
| I-6 | UB | RW | 845.4 | 10.75 (s, 1H), 8.77 (d, J = 4.3 Hz, 1H), 8.61 (s, 1H), 7.91 (s, |
| | | | | 1H), 7.87-7.73 (m, 3H), 7.70-7.64 (m, 1H), 7.38-7.28 (m, 2H), |
| | | | | 6.78-6.71 (m, 2H), 6.64-6.58 (m, 2H), 5.93 (s, 1H), 5.37 (d, J = |
| | | | | 7.3 Hz, 1H), 4.23-4.13 (m, 3H), 3.68 (s, 2H), 3.48 (t, J = 8.4 Hz, |
| | | | | 2H), 3.08-3.01 (m, 1H), 2.99-2.80 (m, 15H), 2.75-2.66 (m, 1H), |
| | | | | 2.43-2.28 (m, 1H), 2.25-2.16 (m, 1H), 2.15-2.05 (m, 1H), 1.91- |
| | | | | 1.75 (m, 3H), 0.82-0.75 (m, 2H), 0.60-0.54 (m, 2H). |
| I-62 | UF | QG | 906.4 | 11.05 (s, 1H), 8.78-8.71 (m, 1H), 7.90 (s, 1H), 7.68-7.58 (m, |
| | | | | 2H), 7.21-7.13 (m, 1H), 6.93 (d, J = 8.6 Hz, 1H), 6.86 (d, J = |
| | | | | 7.6 Hz, 1H), 6.84-6.79 (m, 1H), 6.65-6.58 (m, 1H), 5.93-5.88 |
| | | | | (m, 1H), 5.29 (dd, J = 12.9, 5.4 Hz, 1H), 4.17 (t, J = 8.7 Hz, 2H), |
| | | | | 3.58-3.45 (m, 3H), 3.31 (s, 3H), 3.20 (t, J = 8.6 Hz, 2H), 3.10- |
| | | | | 3.01 (m, 5H), 2.98-2.83 (m, 9H), 2.70-2.59 (m, 6H), 2.37-2.10 |
| | | | | (m, 4H), 2.03-1.95 (m, 1H), 1.82-1.69 (m, 4H), 1.43- |
| | | | | 1.31 (m, 2H), 0.83-0.75 (m, 2H), 0.59-0.52 (m, 2H) |
| I-647 | PX | NP | 894.3 | 10.55 (s, 1H), 9.05 (d, J = 8.8 Hz, 1H), 8.60 (d, J = 2.1 Hz, 1H), |
| | | | | 8.25 (s, 1H), 7.93 (s, 1H), 7.91-7.86 (m, 1H), 7.80 (dd, J = 8.2, |
| | | | | 2.2 Hz, 1H), 7.76-7.65 (m, 3H), 7.58 (s, 1H), 7.39 (dd, J = 8.2, |
| | | | | 2.0 Hz, 1H), 7.36-7.27 (m, 2H), 5.95 (s, 1H), 4.39-4.31 (m, |
| | | | | 1H), 4.19 (t, J = 8.5 Hz, 2H), 3.93-3.86 (m, 1H), 3.81-3.74 (m, |
| | | | | 2H), 3.72-3.60 (m, 5H), 3.46 (t, J = 8.5 Hz, 2H), 3.32-3.29 (m, |
| | | | | 2H), 3.21 (s, 3H), 3.19-3.15 (m, 2H), 2.97 (d, J = 4.9 Hz, 3H), |
| | | | | 2.80-2.70 (m, 2H), 2.17-1.98 (m, 4H), 1.61-1.36 (m, 2H) |
| I-662 | PX | SJ | 886.3 | 10.52 (s, 1H), 8.75 (d, J = 4.4 Hz, 1H), 8.61 (s, 1H), 7.98 |
| | | | | (d, J = 1.6 Hz, 1H), 7.85-7.63 (m, 5H), 7.57 (s, 1H), 7.39 (d, J = 8.0 |
| | | | | Hz, 1H), 7.16-7.05 (m, 1H), 6.02-5.91 (m, 1H), 4.98-4.71 |
| | | | | (m, 1H), 4.31-4.00 (m, 3H), 3.97-3.57 (m, 7H), 3.49-3.36 |
| | | | | (m, 4H), 3.23-3.11 (m, 2H), 3.08-2.93 (m, 4H), 2.84- |
| | | | | 2.65 (m, 2H), 2.11-1.94 (m, 2H), 1.29-1.11 (m, 1H), |
| | | | | 1.06-0.88 (m, 1H) |
| I-68 | T | RY | 781.4 | 11.05 (s, 1H), 8.76 (d, J = 4.4 Hz, 1H), 8.66 (d, J = 4.8 Hz, 1H), |
| | | | | 7.91 (s, 1H), 7.85-7.79 (m, 1H), 7.73 (s, 1H), 7.70-7.61 (m, |
| | | | | 1H), 7.44-7.28 (m, 3H), 6.95 (d, J = 8.4 Hz, 1H), 6.86 (d, J = |
| | | | | 2.4 Hz, 1H), 6.65 (dd, J = 8.4, 2.4 Hz, 1H), 5.94 (d, J = 2.8 Hz, |
| | | | | 1H), 5.36-5.23 (m, 1H), 4.18 (t, J = 8.4 Hz, 2H), 3.69 (s, 2H), |
| | | | | 3.47 (t, J = 8.4 Hz, 2H), 3.38-3.33 (m, 4H), 3.21-3.11 (m, |
| | | | | 4H), 3.02-2.83 (m, 5H), 2.76-2.59 (m, 5H), 2.05-1.96 (m, |
| | | | | 1H), 0.85-0.73 (m, 2H), 0.62-0.51 (m, 2H) |
| I-71 | UI | RY | 799.4 | 11.10 (s, 1H), 8.77 (s, 1H), 8.66 (d, J = 5.2 Hz, 1H), 7.91 (s, |
| | | | | 1H), 7.87-7.78 (m, 1H), 7.73 (s, 1H), 7.70-7.63 (m, 1H), |
| | | | | 7.42-7.28 (m, 3H), 6.87 (d, J = 8.4 Hz, 1H), 6.76 (t, J = 8.0 Hz, 1H), |
| | | | | 5.94 (d, J = 4.8 Hz, 1H), 5.39-5.29 (m, 1H), 4.23-4.12 (m, |
| | | | | 2H), 3.75-3.66 (m, 2H), 3.56-3.43 (m, 4H), 3.07-3.00 (m, |
| | | | | 3H), 2.99-2.83 (m, 5H), 2.75-2.59 (m, 7H), 2.48-2.30 (m, |
| | | | | 1H), 2.06-1.93 (m, 1H), 0.98-0.91 (m, 2H), 0.58-0.56 |
| | | | | (m, 2H) |
| I-710 | ST | KO | 903.3 | 10.49 (s, 1H), 8.86 (s, 1H), 7.93 (s, 1H), 7.70-7.60 (m, 2H), |
| | | | | 7.58-7.51 (m, 2H), 7.41-7.35 (m, 1H), 7.16-7.08 (m, 1H), |
| | | | | 6.86-6.77 (m, 1H), 5.98 (s, 1H), 4.97-4.76 (m, 1H), 4.16-4.09 (m, |
| | | | | 2H), 3.71-3.50 (m, 8H), 3.20-3.09 (m, 3H), 3.02-2.96 (m, 4H), |
| | | | | 2.86-2.72 (m, 6H), 2.38-2.27 (m, 5H), 1.73-1.56 (m, 2H), |
| | | | | 1.53-1.45 (m, 1H), 1.29-1.15 (m, 4H), 1.04-0.73 (m, 3H) |
| I-716 | TM | KO | 931.3 | 10.50 (s, 1H), 8.88 (d, J = 4.7 Hz, 1H), 7.95 (s, 1H), 7.71-7.61 |
| | | | | (m, 2H), 7.61-7.54 (m, 2H), 7.40 (dd, J = 8.2, 2.1 Hz, 1H), 7.14 |
| | | | | (t, J = 7.8 Hz, 1H), 6.85 (d, J = 7.5 Hz, 1H), 6.00 (s, 1H), 5.00- |
| | | | | 4.77 (m, 1H), 4.14 (t, J = 8.6 Hz, 2H), 3.79-3.74 (m, 1H), 3.67- |
| | | | | 3.55 (m, 3H), 3.54-3.49 (m, 2H), 3.32-3.30 (m, 4H), 3.17 (t, J = |
| | | | | 8.4 Hz, 2H), 3.08-3.00 (m, 1H), 2.98-2.93 (m, 4H), 2.92-2.85 |
| | | | | (m, 2H), 2.82-2.70 (m, 3H), 2.46-2.22 (m, 6H), 2.19-2.09 (m, |
| | | | | 3H), 1.86-1.61 (m, 4H), 1.53-1.40 (m, 1H), 1.26-1.19 (m, 1H), |
| | | | | 1.10-1.01 (m, 2H), 1.00-0.89 (m, 1H) |
| I-72 | Q0 | KO | 896.4 | 11.06 (s, 1H), 8.93-8.82 (m, 1H), 8.20 (s, 1H), 7.95 (s, 1H), 7.72- |
| | | | | 7.62 (m, 1H), 7.60-7.51 (m, 1H), 7.13 (t, J = 7.8 Hz, 1H), 6.98- |
| | | | | 6.91 (m, 1H), 6.88-6.78 (m, 2H), 6.65-6.58 (m, 1H), 5.97 (s, |
| | | | | 1H), 5.29 (dd, J = 13.0, 5.3 Hz, 1H), 5.00-4.74 (m, 1H), 4.12 (t, |
| | | | | J = 8.7 Hz, 2H), 3.58-3.43 (m, 3H), 3.40-3.35 (m, 1H), 3.31 (s, |
| | | | | 3H), 3.21-3.12 (m, 2H), 3.11-3.04 (m, 4H), 2.99-2.80 (m, 8H), |
| | | | | 2.72-2.56 (m, 3H), 2.51 (s, 3H), 2.43-2.36 (m, 4H), 2.34-2.26 |
| | | | | (m, 1H), 2.04-1.93 (m, 1H), 1.75-1.64 (m, 1H), 1.58-1.45 (m, |
| | | | | 1H), 1.30-1.15 (m, 1H), 1.04-0.90 (m, 1H) |
| I-725 | PX | TG | 865.3 | 10.52 (s, 1H), 8.78 (d, J = 8.8 Hz, 1H), 8.63-8.60 (m, 1H), 8.18 |
| | | | | (d, J = 9.9 Hz, 1H), 8.13-8.10 (m, 2H), 7.85-7.79 (m, 1H), 7.77- |
| | | | | 7.73 (m, 1H), 7.69 (d, J = 8.2 Hz, 1H), 7.57 (s, 1H), 7.42-7.37 |
| | | | | (m, 4H), 4.43-4.34 (m, 1H), 4.26 (t, J = 8.5 Hz, 2H), 3.97-3.87 |
| | | | | (m, 1H), 3.83-3.65 (m, 7H), 3.52 (t, J = 8.4 Hz, 2H), 3.44-3.37 |
| | | | | (m, 1H), 3.35-3.33 (m, 2H), 3.23 (s, 3H), 3.20-3.16 (m, 2H), |
| | | | | 2.78-2.73 (m, 2H), 2.15-2.04 (m, 4H), 1.57-1.48 (m, 2H) |
| I-766 | MU | KO | 916.4 | 10.52 (s, 1H), 8.88 (d, J = 4.7 Hz, 1H), 7.95 (s, 1H), 7.73-7.66 |
| | | | | (m, 1H), 7.64 (d, J = 8.2 Hz, 1H), 7.61-7.52 (m, 2H), 7.39 (dd, |
| | | | | J = 8.2, 2.0 Hz, 1H), 7.14 (t, J = 7.8 Hz, 1H), 6.85 (d, J = 7.6) |
| | | | | Hz, 1H), 6.00 (s, 1H), 5.00-4.78 (m, 1H), 4.14 (t, J = 8.7 Hz, |
| | | | | 2H), 3.79-3.74 (m, 1H), 3.67-3.56 (m, 3H), 3.55-3.50 (m, 2H), |
| | | | | 3.42-3.36 (m, 2H), 3.31-3.24 (m, 2H), 3.17 (t, J = 8.5 Hz, 2H), |
| | | | | 3.08-3.01 (m, 1H), 2.95 (d, J = 4.9 Hz, 3H), 2.83-2.71 (m, 3H), |
| | | | | 2.46-2.37 (m, 2H), 2.36-2.19 (m, 4H), 2.17-2.06 (m, 2H), 1.94- |
| | | | | 1.86 (m, 1H), 1.56-1.42 (m, 6H), 1.41-1.30 (m, 3H), |
| | | | | 1.27-1.17 (m, 1H), 1.05-0.90 (m, 1H) |
| I-80 | UL | QX | 918.4 | 11.06 (s, 1H), 8.88 (d, J = 4.7 Hz, 1H), 8.61 (d, J = 2.1 Hz, 1H), |
| | | | | 8.28 (s, 1H), 7.96 (s, 1H), 7.84-7.80 (m, 1H), 7.77-7.73 (m, 2H), |
| | | | | 7.70-7.66 (m, 1H), 7.37-7.25 (m, 2H), 6.93 (d, J = 8.5 Hz, 1H), |
| | | | | 6.83 (d, J = 2.2 Hz, 1H), 6.62 (dd, J = 8.6, 2.2 Hz, 1H), 6.00 (s, |
| | | | | 1H), 5.29 (dd, J = 13.0, 5.3 Hz, 1H), 4.98-4.78 (m, 1H), 4.14 (t, |
| | | | | J = 8.5 Hz, 2H), 3.67 (s, 2H), 3.45 (t, J = 8.6 Hz, 2H), 3.30 (s, |
| | | | | 3H), 3.09-3.03 (m, 6H), 3.00-2.83 (m, 10H), 2.76-2.57 (m, |
| | | | | 2H), 2.40-2.30 (m, 1H), 2.23-2.18 (m, 1H), 2.04-1.95 (m, 1H), |
| | | | | 1.91-1.76 (m, 2H), 1.27-1.18 (m, 1H), 1.03-0.93 (m, 1H) |
| I-81d | UI | RY | 759.2 | 11.09 (s, 1H), 8.66 (s, 1H), 8.26 (s, 1H), 7.92 (s, 1H), 7.88-7.82 |
| | | | | (m, 2H), 7.72 (s, 1H), 7.66-7.62 (m, 1H), 7.40-7.28 (m, 3H), |
| | | | | 6.87 (d, J = 8.5 Hz, 1H), 6.77 (t, J = 8.2 Hz, 1H), 5.99 (s, 1H), |
| | | | | 5.34 (dd, J = 12.8, 5.3 Hz, 1H), 4.19 (t, J = 8.4 Hz, 2H), 3.69 (s, |
| | | | | 2H), 3.54-3.37 (m, 5H), 3.30 (s, 3H), 3.05-2.94 (m, 5H), |
| | | | | 2.94-2.85 (m, 1H), 2.75-2.62 (m, 5H), 2.02-1.97 (m, 1H) |
| I-852 | PX | QB | 882.2 | 10.51 (s, 1H), 8.96 (d, J = 4.4 Hz, 1H), 8.59 (d, J = 2.1 Hz, 1H), |
| | | | | 7.96 (s, 1H), 7.79 (dd, J = 8.2, 2.2 Hz, 1H), 7.74-7.66 (m, 3H), |
| | | | | 7.62-7.54 (m, 2H), 7.39 (dd, J = 8.3, 2.0 Hz, 1H), 7.14 (s, 1H), |
| | | | | 6.02 (s, 1H), 4.97-4.70 (m, 1H), 4.12 (t, J = 8.3 Hz, 2H), 3.96- |
| | | | | 3.83 (m, 1H), 3.82-3.72 (m, 2H), 3.70 (s, 2H), 3.68-3.58 (m, |
| | | | | 2H), 3.44-3.32 (m, 5H), 3.21-3.14 (m, 2H), 3.08-3.00 (m, 1H), |
| | | | | 2.97 (d, J = 4.9 Hz, 3H), 2.79-2.71 (m, 2H), 2.39 (s, 3H), 2.08- |
| | | | | 1.99 (m, 2H), 1.23-1.15 (m, 1H), 0.92-0.83 (m, 1H) |
| I-859 | PX | PZ | 898.3 | 10.52 (s, 1H), 8.88 (d, J = 4.7 Hz, 1H), 8.60 (d, J = 2.2 Hz, 1H), |
| | | | | 7.96 (s, 1H), 7.80 (dd, J = 8.2, 2.2 Hz, 1H), 7.77-7.65 (m, 3H), |
| | | | | 7.57 (s, 1H), 7.43-7.36 (m, 2H), 6.86 (d, J = 2.2 Hz, 1H), 6.03 |
| | | | | (s, 1H), 4.95-4.72 (m, 1H), 4.14 (t, J = 8.3 Hz, 2H), 3.95-3.88 |
| | | | | (m, 1H), 3.84 (s, 3H), 3.80-3.73 (m, 1H), 3.70 (s, 2H), 3.67-3.58 |
| | | | | (m, 2H), 3.43-3.31 (m, 6H), 3.21-3.14 (m, 2H), 3.06-2.98 (m, |
| | | | | 1H), 2.96 (d, J = 4.9 Hz, 3H), 2.79-2.70 (m, 2H), 2.09-1.98 (m, |
| | | | | 2H), 1.22-1.13 (m, 1H), 1.04-0.92 (m, 1H) |
| I-869 | RN | KP | 804.4 | 10.17 (s, 1H), 9.11 (s, 1H), 8.74 (s, 1H), 8.53 (s, 1H), 8.32-8.26 |
| | | | | (m, 1H), 7.71 (s, 1H), 7.11 (s, 1H), 7.05-6.94 (m, 1H), 6.94-6.83 |
| | | | | (m, 2H), 5.41-5.29 (m, 1H), 4.62-4.49 (m, 1H), 4.26-4.17 (m, |
| | | | | 1H), 4.11-3.86 (m, 4H), 3.65 (s, 3H), 3.20-3.13 (m, 2H), 3.12 |
| | | | | (s, 3H), 3.10-3.00 (m, 2H), 2.93-2.81 (m, 1H), 2.81-2.57 (m, |
| | | | | 5H), 2.48-2.40 (m, 1H), 2.38 (s, 3H), 2.35-2.29 (m, 2H), 2.28- |
| | | | | 2.13 (m, 3H), 2.10 (s, 3H), 2.07-1.96 (m, 3H), 1.94-1.84 (m, |
| | | | | 2H), 1.79-1.63 (m, 1H), 1.48-1.29 (m, 2H) |
| I-872b | RN | RO | 790.3 | 11.07 (s, 1H), 10.20 (s, 1H), 9.11 (s, 1H), 8.77-8.73 (m, 1H), |
| | | | | 8.51 (s, 1H), 8.15 (s, 0.3H), 7.69 (s, 1H), 7.12 (s, 1H), 6.95 (d, |
| | | | | J = 8.6 Hz, 1H), 6.87 (d, J = 2.2 Hz, 1H), 6.67 (dd, J = 8.6, 2.2 |
| | | | | Hz, 1H), 5.30 (dd, J = 12.9, 5.4 Hz, 1H), 4.64-4.53 (m, 1H), |
| | | | | 4.47-4.40 (m, 1H), 4.21 (dd, J = 9.6, 1.3 Hz, 1H), 4.08-3.96 (m, |
| | | | | 2H), 3.91 (d, J = 9.7 Hz, 1H), 3.74-3.67 (m, 2H), 3.43-3.37 (m, |
| | | | | 2H), 3.19-3.14 (m, 1H), 3.12 (s, 3H), 2.95-2.85 (m, 1H), 2.82- |
| | | | | 2.63 (m, 5H), 2.62-2.58 (m, 1H), 2.55 (s, 3H), 2.48-2.42 (m, |
| | | | | 2H), 2.38 (s, 3H), 2.14-2.06 (m, 6H), 2.04-1.86 (m, |
| | | | | 3H), 1.76-1.62 (m, 2H) |
| I-876 | PX | VS | 902.2 | 10.59-10.46 (m, 1H), 8.84-8.71 (m, 1H), 8.65-8.54 (m, 1H), |
| | | | | 8.33(s, 0.52H), 8.03-7.93 (m, 1H), 7.85-7.67 (m, 5H), 7.62-7.53 |
| | | | | (m, 1H), 7.42-7.28 (m, 2H), 6.01-5.88 (m, 1H), 4.97-4.70 (m, |
| | | | | 1H), 4.21-4.12 (m, 2H), 3.91-3.77 (m, 4H), 3.76-3.70 (m, 3H), |
| | | | | 3.44-3.30 (m, 6H), 3.21-3.14 (m, 2H), 3.01-2.94 (m, 3H), 2.81- |
| | | | | 2.72 (m, 2H), 2.10-2.00 (m, 2H), 1.27-1.17 (m, 1H), |
| | | | | 1.01-0.88 (m, 1H) |
| I-88 | NK | QX | 918.4 | 11.06 (s, 1H), 8.88 (d, J = 4.7 Hz, 1H), 8.61 (d, J = 2.1 Hz, 1H), |
| | | | | 8.28 (s, 1H), 7.96 (s, 1H), 7.84-7.80 (m, 1H), 7.77-7.73 (m, 2H), |
| | | | | 7.70-7.66 (m, 1H), 7.37-7.25 (m, 2H), 6.93 (d, J = 8.5 Hz, 1H), |
| | | | | 6.83 (d, J = 2.2 Hz, 1H), 6.62 (dd, J = 8.6, 2.2 Hz, 1H), 6.00 (s, |
| | | | | 1H), 5.29 (dd, J = 13.0, 5.3 Hz, 1H), 4.98-4.78 (m, 1H), 4.14 (t, |
| | | | | J = 8.5 Hz, 2H), 3.67 (s, 2H), 3.45 (t, J = 8.6 Hz, 2H), 3.30 (s, |
| | | | | 3H), 3.09-3.03 (m, 6H), 3.00-2.83 (m, 10H), 2.76-2.57 (m, |
| | | | | 2H), 2.40-2.30 (m, 1H), 2.23-2.18 (m, 1H), 2.04-1.95 (m, 1H), |
| | | | | 1.91-1.76 (m, 2H), 1.27-1.18 (m, 1H), 1.03-0.93 (m, 1H) |
| I-1060 | TB | MN | 928.5 | δ 10.39 (s, 1H), 9.03 (d, J = 8.8 Hz, 1H), 7.91 (s, 1H), 7.74 |
| | | | | 7.61 (m, 2H), 7.25 (d, J = 8.8 Hz, 1H), 7.16 (t, J = 7.6 Hz, 1H), |
| | | | | 6.93-6.80 (m, 2H), 6.72 (dd, J = 9.2, 2.8 Hz, 1H), 5.93 (d, J = |
| | | | | 3.2 Hz, 1H), 4.41-4.27 (m, 1H), 4.19 (t, J = 8.4 Hz, 2H), 3.83- |
| | | | | 3.63 (m, 2H), 3.62-3.45 (m, 4H), 3.26-3.12 (m, 5H), 3.02- |
| | | | | 2.84 (m, 5H), 2.83-2.60 (m, 9H), 2.34-2.23 (m, 1H), 2.19- |
| | | | | 1.99 (m, 3H), 1.89-1.34 (m, 12H), 1.34-1.11 (m, 5H) |

^{a}The reductive amination was run from 25-50° C. under standard conditions for 0.5-16 h. KOAc or TEA were added to free base amines when necessary. Purification was run under a variety of typical methods including prep-HPLC, reverse phase chromatography, and silica gel chromatography. DCE could also be used as a cosolvent.
^{b}Ketone was used in place of the aldehyde.
^{c}The product of the reductive amination was further deprotected with TFA in DCM at rt for 2 h.
^{d}The product of the reductive amination was further deprotected with TFA in TfOH at 70° C. for 1 h.

### Example 5 (Method 3): Synthesis of 6-(4-(5-((7-(3-chloro-5-(2,4-dioxotetrahydropyrimidin-1(2H)-yl)-4-methylbenzoyl)-5,5-difluoro-2,7-diazaspiro[3.5]nonan-2-yl)methyl)-3-fluoropyridin-2-yl)indolin-1-yl)-N-((1R,2S)-2-fluorocyclopropyl)-8-(methylamino)imidazo[1,2-b]pyridazine-3-carboxamide (I-1008)

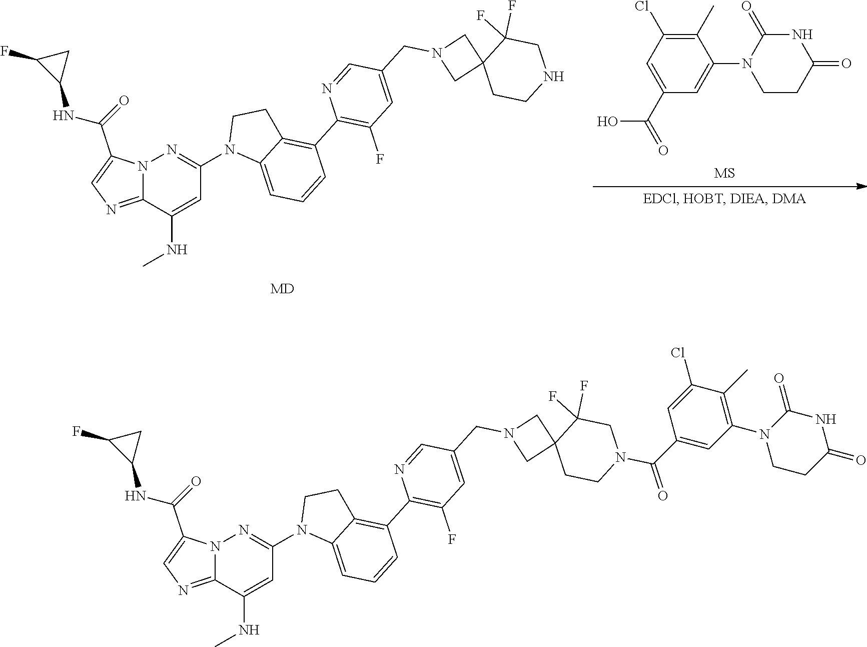

To a stirred solution of 6-{4-[5-({5,5-difluoro-2,7-diazaspiro[3.5]nonan-2-yl}methyl)-3-fluoropyridin-2-yl]-2,3-dihydroindol-1-yl}-N-[(1R,2S)-2-fluorocyclopropyl]-8-(methylamino)imidazo[1,2-b]pyridazine-3-carboxamide trifluoroacetate (60.00 mg, 0.08 mmol, Intermediate MD) and 3-chloro-5-(2,4-dioxo-1,3-diazinan-1-yl)-4-methylbenzoic acid (23.12 mg, 0.08 mmol, Intermediate MS) and DIEA (31.71 mg, 0.24 mmol) in DMA (2 mL) were added HOBT (11.05 mg, 0.08 mmol) and EDCI (15.68 mg, 0.08 mmol) at 0° C. under nitrogen atmosphere. The resulting mixture was stirred for 2 h at rt under nitrogen atmosphere. After completion of the reaction, the reaction mixture was purified directly by reverse phase flash chromatography (Column: WelFlash™ C18-I, 20-40 μm, 120 g; Eluent A: Water (plus 10 mmol/L NH₄HCO₃); Eluent B: ACN; Gradient: 30%-50% B in 30 min; Flow rate: 60 mL/min; Detector: 220/254 nm; desired fractions were collected at 36% B) and concentrated under reduced pressure to afford the title compound (12.90 mg, 17% yield) as a white solid. LC/MS (ESI, m/z): [(M+H)]⁺=900.3. ¹H NMR (400 MHz, DMSO-d₆) δ 10.47 (s, 1H), 8.90-8.85 (m, 1H), 8.48 (s, 1H), 7.96 (s, 1H), 7.81-7.69 (m, 3H), 7.48-7.42 (m, 1H), 7.41-7.36 (m, 1H), 7.32-7.27 (m, 1H), 7.13-7.07 (m, 1H), 6.05-5.98 (m, 1H), 5.01-4.76 (m, 1H), 4.14 (t, J=8.5 Hz, 2H), 3.93-3.79 (m, 2H), 3.75 (s, 2H), 3.70-3.52 (m, 3H), 3.25-3.15 (m, 4H), 3.12-3.01 (m, 2H), 2.97 (d, J=4.9 Hz, 3H), 2.89-2.78 (m, 1H), 2.75-2.66 (m, 1H), 2.26 (s, 3H), 2.10-1.99 (m, 2H), 1.44-1.36 (m, 1H), 1.29-1.16 (m, 2H), 1.06-0.91 (m, 1H).

TABLE 5
Compounds synthesized via Method 3, via the coupling of the corresponding amines.

| | | | LCMS | |
|---|---|---|---|---|
| | | | (ESI+) | |
| | | | m/z | |
| I-#^{a} | Amine | Acid | (M + H)⁺ | ¹H NMR (400 MHz, DMSO-d6) |
| I-730 | NJ | NH | 855.1 | 10.38 (s, 1H), 9.05 (d, J = 8.8 Hz, 1H), 8.52 (d, J = 2.4 Hz, 1H), 7.92 |
| | | | | (s, 1H), 7.90-7.82 (m, 1H), 7.75-7.64 (m, 3H), 7.38-7.29 (m, 3H), |
| | | | | 7.24 (d, J = 10.0 Hz, 1H), 5.96 (s, 1H), 4.44-4.27 (m, 1H), 4.19 (t, |
| | | | | J = 8.4 Hz, 2H), 3.84-3.69 (m, 2H), 3.65-3.40 (m, 5H), 3.25-3.06 |
| | | | | (m, 5H), 2.96 (d, J = 4.8 Hz, 3H), 2.86-2.53 (m, 5H), 2.21 (s, 3H), |
| | | | | 2.15-1.83 (m, 4H), 1.69-1.38 (m, 8H) |
| I-991 | OE | ME | 896.3 | 10.51 (s, 1H), 8.92 (d, J = 7.9 Hz, 1H), 8.48 (s, 1H), 7.96-7.88 (m, |
| | | | | 2H), 7.76 (dd, J = 11.2, 1.6 Hz, 1H), 7.69 (q, J = 4.9 Hz, 1H), 7.40 (s, |
| | | | | 2H), 7.32 (t, J = 7.9 Hz, 1H), 7.15-7.08 (m, 1H), 5.93 (s, 1H), 4.59- |
| | | | | 4.48 (m, 1H), 4.19 (t, J = 8.5 Hz, 2H), 3.94-3.84 (m, 1H), 3.79-3.72 (m, |
| | | | | 3H), 3.69-3.63 (m, 2H), 3.44-3.29 (m, 4H), 3.27-3.17 (m, 4H), 2.96 |
| | | | | (d, J = 4.9 Hz, 3H), 2.84-2.66 (m, 2H), 2.42 (s, 3H), |
| | | | | 2.38-2.25 (m, 2H), 2.07-1.90 (m, 4H), 1.76-1.68 (m, 2H) |
| I-982 | OH | ME | 882.3 | 10.49 (s, 1H), 8.74 (d, J = 4.4 Hz, 1H), 8.48 (s, 1H), 7.91 (s, 1H), |
| | | | | 7.87-7.60 (m, 3H), 7.46-7.34 (m, 2H), 7.32 (t, J = 7.6 Hz, 1H), 7.11 (dd, |
| | | | | J = 7.6, 2.0 Hz, 1H), 5.98-5.89 (m, 1H), 4.24-4.11 (m, 2H), 3.98- |
| | | | | 3.82 (m, 1H), 3.81-3.58 (m, 6H), 3.46-3.36 (m, 3H), 3.26-3.15 (m, |
| | | | | 4H), 3.04-2.88 (m, 4H), 2.81-2.70 (m, 2H), 2.42 (s, 3H), 2.14-1.99 |
| | | | | (m, 2H), 0,85-0,75 (m, 2H), 0,63-0,52 (m, 2H) |
| I-1023 | MD | MM | 886.3 | 10.52 (s, 1H), 8.87 (d, J = 4.7 Hz, 1H), 8.51-8.46 (m, 1H), 7.97 (s, 1H), |
| | | | | 7.83-7.65 (m, 4H), 7.57 (s, 1H), 7.40 (dd, J = 8.1, 2.0 Hz, 1H), 7.30 (t, |
| | | | | J = 7.8 Hz, 1H), 7.13-7.07 (m, 1H), 6.02 (s, 1H), 4.99-4.78 (m, 1H), |
| | | | | 4.15 (t, J = 8.5 Hz, 2H), 3.99-3.83 (m, 1H), 3.82-3.72 (m, 3H), 3.70- |
| | | | | 3.59 (m, 2H), 3.47-3.34 (m, 4H), 3.25-3.16 (m, 4H), 3.07-3.01 (m, 1H), |
| | | | | 2.97 (d, J = 4.9 Hz, 3H), 2.80-2.71 (m, 2H), 2.11-2.01 (m, 2H), |
| | | | | 1.28-1.19 (m, 1H), 1.03-0.92 (m, 1H) |
| I-357 | UG | RI | 848.4 | 10.38 (s, 1H), 8.87 (d, J = 4.8 Hz, 1H), 8.59 (d, J = 2.0 Hz, 1H), 7.96 |
| | | | | (s, 1H), 7.91-7.58 (m, 4H), 7.46-7.19 (m, 5H), 5.98 (s, 1H), 5.06- |
| | | | | 4.68 (m, 1H), 4.12 (t, J = 8.4 Hz, 2H), 3.93-3.49 (m, 7H), 3.42 (t, J = |
| | | | | 8.4 Hz, 2H), 3.31-3.23 (m, 3H), 3.22-3.12 (m, 2H), 3.11-3.00 (m, |
| | | | | 1H), 2.96 (d, J = 4.8 Hz, 3H), 2.88-2.63 (m, 2H), 2.24 (s, 3H), |
| | | | | 2.11-1.95 (m, 2H), 1.33-1.13 (m, 1H), 1.06-0.90 (m, 1H) |
| I-648 | SO | MM | 886.3 | 10.53 (s, 1H), 8.99 (s, 1H), 8.60 (s, 1H), 7.98 (s, 1H), 7.85-7.77 (m, |
| | | | | 2H), 7.76-7.64 (m, 3H), 7.57 (s, 1H), 7.39 (dd, J = 8.4, 2.0 Hz, 1H), |
| | | | | 7.35-7.24 (m, 2H), 6.07-5.90 (m, 1H), 4.22-4.06 (m, 2H), 4.00- |
| | | | | 3.54 (m, 9H), 3.52-3.39 (m, 4H), 3.24-3.11 (m, 2H), 2.97 (d, J = 4.8 |
| | | | | Hz, 3H), 2.84-2.69 (m, 2H), 2.14-1.97 (m, 3H), 1.68-1.54 (m, 1H) |
| I-649 | SQ | MM | 886.4 | 10.53 (s, 1H), 9.00 (s, 1H), 8.60 (s, 1H), 7.98 (s, 1H), 7.88-7.78 (m, |
| | | | | 2H), 7.77-7.65 (m, 3H), 7.57 (s, 1H), 7.39 (dd, J = 8.4, 2.0 Hz, 1H), |
| | | | | 7.35-7.25 (m, 2H), 6.00 (d, J = 6.0 Hz, 1H), 4.22-4.09 (m, 2H), 4.02- |
| | | | | 3.83 (m, 1H), 3.82-3.55 (m, 8H), 3.44 (t, J = 8.4 Hz, 2H), 3.31- |
| | | | | 3.27 (m, 2H), 3.23-3.13 (m, 2H), 2.97 (d, J = 5.2 Hz, 3H), 2.80-2.71 |
| | | | | (m, 2H), 2.13-1.98 (m, 3H), 1.67-1.56 (m, 1H) |
| I-760 | TV | ME | 846.3 | 10.48 (s, 1H), 8.97-8.92 (m, 1H), 8.86 (d, J = 4.7 Hz, 1H), 8.65-8.61 |
| | | | | (m, 1H), 7.97 (s, 1H), 7.81 (d, J = 7.7 Hz, 1H), 7.76-7.68 (m, 1H), 7.41- |
| | | | | 7.28 (m, 4H), 6.01 (s, 1H), 5.00-4.77 (m, 1H), 4.17 (t, J = 8.5 Hz, 2H), |
| | | | | 3.79-3.67 (m, 1H), 3.66-3.58 (m, 1H), 3.48-3.41 (m, 3H), 3.29-3.18 (m, |
| | | | | 1H), 3.08-3.01 (m, 1H), 3.00-2.94 (m, 5H), 2.82-2.68 (m, 4H), 2.41 (s, |
| | | | | 3H), 2.05-1.95 (m, 2H), 1.72-1.43 (m, 7H), 1.24-1.19 (m, |
| | | | | 1H), 1.03-0.91 (m, 1H) |
| I-777 | MO | MS | 942.5 | 10.45 (s, 1H), 9.02 (d, J = 8.8 Hz, 1H), 7.90 (s, 1H), 7.73-7.61 (m, |
| | | | | 2H), 7.43 (d, J = 1.6 Hz, 1H), 7.35 (d, J = 1.6 Hz, 1H), 7.16 (t, J = 7.6 |
| | | | | Hz, 1H), 6.86 (d, J = 7.6 Hz, 1H), 5.93 (s, 1H), 4.41-4.27 (m, 1H), |
| | | | | 4.19 (t, J = 8.4 Hz, 2H), 3.89-3.70 (m, 2H), 3.62-3.46 (m, 5H), 3.31- |
| | | | | 3.25 (m, 3H), 3.25-3.14 (m, 6H), 3.01-2.77 (m, 7H), 2.75-2.59 |
| | | | | (m, 6H), 2.37-2.25 (m, 1H), 2.18-1.99 (m, 3H), 1.87-1.66 |
| | | | | (m, 2H), 1.60-1.33 (m, 10H) |
| I-799 | RU | ME | 911.4 | 10.47 (s, 1H), 8.90-8.81 (m, 3H), 7.96 (s, 1H), 7.90-7.81 (m, 2H), 7.73- |
| | | | | 7.66 (m, 1H), 7.43-7.38 (m, 2H), 7.33 (t, J = 7.9 Hz, 1H), 6.01 (s, 1H), |
| | | | | 4.98-4.77 (m, 1H), 4.29-4.11 (m, 3H), 3.81-3.56 (m, 6H), 3.54-3.42 (m, |
| | | | | 1H), 3.28-3.20 (m, 1H), 3.16-3.02 (m, 2H), 2.97 (d, J = 4.9 Hz, 3H), |
| | | | | 2.83-2.69 (m, 4H), 2.59-2.54 (m, 2H), 2.41 (s, 3H), 1.92-1.80 (m, 2H), |
| | | | | 1.75-1.51 (m, 4H), 1.23-1.17 (m, 1H), 1.03-0.91 (m, 1H) |
| I-823 | TP | MM | 864.4 | 10.50 (s, 1H), 8.86 (d, J = 4.7 Hz, 1H), 7.94 (s, 1H), 7.69-7.60 (m, 2H), |
| | | | | 7.60-7.49 (m, 2H), 7.41 (dd, J = 8.2, 2.0 Hz, 1H), 7.11 (t, J = 7.8 Hz, |
| | | | | 1H), 6.70 (d, J = 7.5 Hz, 1H), 5.98 (s, 1H), 5.03-4.72 (m, 1H), 4.52- |
| | | | | 4.36 (m, 1H), 4.14 (t, J = 8.6 Hz, 2H), 3.83-3.71 (m, 1H), 3.70-3.55 (m, |
| | | | | 2H), 3.22 (t, J = 8.4 Hz, 2H), 3.09-2.99 (m, 2H), 2.96 (d, J = 4.9 Hz, |
| | | | | 3H), 2.90-2.68 (m, 4H), 2.58-2.53 (m, 6H), 2.48-2.36 (m, 4H), 2.05 (s, |
| | | | | 6H), 1.93-1.67 (m, 2H), 1.49-1.35 (m, 2H), 1.26-1.15 (m, |
| | | | | 1H), 1.05-0.86 (m, 1H) |
| I-825 | RS | ME | 937.4 | 10.48 (s, 1H), 9.05 (d, J = 8.8 Hz, 1H), 8.90-8.81 (m, 2H), 8.01-7.87 |
| | | | | (m, 3H), 7.76-7.64 (m, 1H), 7.44-7.33 (m, 3H), 5.99 (s, 1H), 4.41-4.30 |
| | | | | (m, 1H), 4.29-4.14 (m, 3H), 3.80-3.68 (m, 6H), 3.65-3.56 (m, 1H), 3.21 |
| | | | | (s, 3H), 3.15-3.04 (m, 1H), 2.98 (d, J = 4.9 Hz, 3H), 2.81-2.73 (m, 4H), |
| | | | | 2.56-2.55 (m, 4H), 2.41 (s, 3H), 2.17-2.09 (m, 1H), 2.07-1.98 (m, 1H), |
| | | | | 1.94-1.79 (m, 2H), 1.73-1.63 (m, 3H), 1.58-1.40 (m, 3H) |

^{a}The coupling was run at rt under standard conditions for 1-2 h. Purification was run under a variety of typical methods including prep-HPLC, reverse phase chromatography, and silica gel chromatography.

### Example 6 (Method 4): 6-(4-(3-(4-(9-(1-(2,6-Dioxopiperidin-3-yl)-3-methyl-2-oxo-2,3-dihydro-LH-benzo[d]imidazol-5-yl)-3,9-diazaspiro[5.5]undecan-3-yl)-3,3-difluoropiperidin-1-yl)prop-1-yn-1-yl)indolin-1-yl)-N-((1R,2S)-2-fluorocyclopropyl)-8-(methylamino)imidazo[1,2-b]pyridazine-3-carboxamide (1-486)

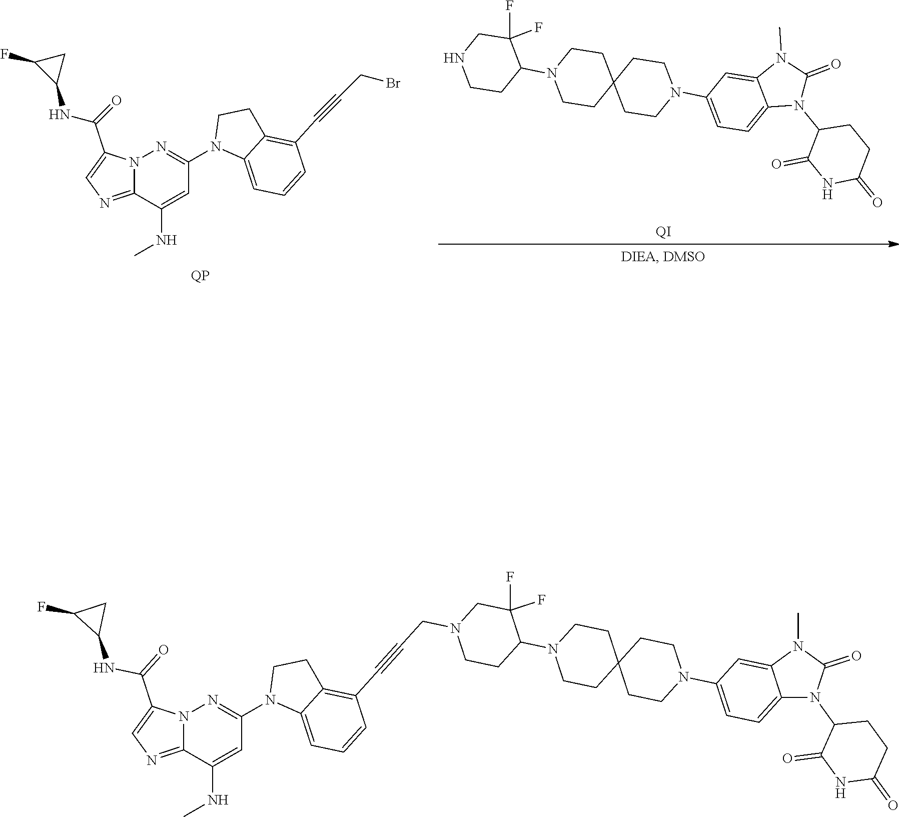

To a stirred mixture of 6-(4-(3-bromoprop-1-yn-1-yl)indolin-1-yl)-N-((1R,2S)-2-fluorocyclopropyl)-8-(methylamino)imidazo[1,2-b]pyridazine-3-carboxamide (20 mg, 0.041 mmol, Intermediate QP) and 3-(5-(9-(3,3-difluoropiperidin-4-yl)-3,9-diazaspiro[5.5]undecan-3-yl)-3-methyl-2-oxo-2,3-dihydro-1H-benzo[d]imidazol-1-yl)piperidine-2,6-dione (22 mg, 0.041 mmol, Intermediate QI) in DMSO (2 mL) was added DIEA (16 mg, 0.12 mmol) at rt. The resulting mixture was stirred for 2 h at 100° C. On completion, the reaction was cooled to rt and purified directly by reverse phase flash chromatography (Column: WelFlash™ C18-I, 20-40 μm, 120 g; Eluent A: Water (plus 10 mmol/L NH₄HCO₃); Eluent B: ACN; Gradient: 25%-55% B in 25 min; Flow rate: 80 mL/min; Detector: 254 nm; desired fractions were collected at 50% B and concentrated under reduced pressure) to afford to give the title compound (10 mg, 24% yield) as a white solid. LC/MS (ESI, m/z): [(M+H)]=933.0. ¹H NMR (400 MHz, DMSO-d₆) δ 11.04 (s, 1H), 8.79 (d, J=4.8 Hz, 1H), 7.95 (s, 1H), 7.75-7.64 (m, 2H), 7.17 (t, J=8.0 Hz, 1H), 6.98 (d, J=7.6 Hz, 1H), 6.92 (d, J=8.4 Hz, 1H), 6.82 (d, J=2.4 Hz, 1H), 6.63 (dd, J=8.4, 2.4 Hz, 1H), 5.96 (s, 1H), 5.28 (dd, J=12.8, 5.2 Hz, 1H), 5.01-4.75 (m, 1H), 4.19 (t, J=8.4 Hz, 2H), 3.77-3.60 (m, 2H), 3.30 (s, 3H), 3.22 (t, J=8.4 Hz, 2H), 3.13-3.01 (m, 6H), 2.99-2.81 (m, 5H), 2.80-2.53 (m, 8H), 2.46-2.32 (m, 1H), 2.05-1.73 (m, 4H), 1.64-1.39 (m, 7H), 1.35-1.13 (m, 1H), 1.03-0.88 (m, 1H).

TABLE 6
Compounds synthesized via Method 4, via the coupling of the corresponding amines and bromides.

| | | | LCMS | |
|---|---|---|---|---|
| | | | (ESI+) | |
| | | | m/z | |
| I-#^{a} | Amine | Bromide | (M + H)⁺ | ¹H NMR (400 MHz, DMSO-d6) |
| I-467 | UN | QP | 947.5 | 11.06 (s, 1H), 8.80 (d, J = 4.8 Hz, 1H), 7.96 (s, 1H), 7.78- |
| | | | | 7.66 (m, 2H), 7.17 (t, J = 8.0 Hz, 1H), 6.98 (d, J = 7.6 Hz, 1H), |
| | | | | 6.92 (d, J = 8.4 Hz, 1H), 6.82 (d, J = 2.4 Hz, 1H), 6.62 (dd, |
| | | | | J = 8.8, 2.4 Hz, 1H), 5.97 (s, 1H), 5.31-5.26 (m, 1H), 5.00-4.76 |
| | | | | (m, 1H), 4.20 (t, J = 8.4 Hz, 2H), 3.74-3.68 (m, 2H), 3.66- |
| | | | | 3.57 (m, 2H), 3.30 (s, 3H), 3.27-3.18 (m, 2H), 3.10-2.99 |
| | | | | (m, 3H), 2.99-2.86 (m, 6H), 2.71-2.62 (m, 2H), 2.61-2.54 |
| | | | | (m, 3H), 2.47-2.38 (m, 3H), 2.35-2.28 (m, 2H), 2.04-1.94 |
| | | | | (m, 1H), 1.90-1.71 (m, 3H), 1.71-1.61 (m, 2H), 1.38-0.90 |
| | | | | (m, 9H) |
| I-473 | QQ | QP | 822.4 | 10.52 (s, 1H), 8.80 (d, J = 4.8 Hz, 1H), 8.19 (s, 0.5H) (FA), |
| | | | | 7.95 (s, 1H), 7.79-7.60 (m, 3H), 7.57 (d, J = 2.0 Hz, 1H), |
| | | | | 7.41 (dd, J = 8.0, 2.0 Hz, 1H), 7.16 (t, J = 8.0 Hz, 1H), 6.95 (d, |
| | | | | J = 7.6 Hz, 1H), 5.95 (s, 1H), 5.02-4.72 (m, 1H), 4.53-4.35 |
| | | | | (m, 1H), 4.17 (t, J = 8.4 Hz, 2H), 3.84-3.71 (m, 1H), 3.69- |
| | | | | 3.52 (m, 5H), 3.19 (t, J = 8.4 Hz, 2H), 3.12-2.97 (m, 2H), |
| | | | | 2.95 (d, J = 4.8 Hz, 3H), 2.86-2.70 (m, 3H), 2.64-2.54 (m, |
| | | | | 8H), 1.93-1.65 (m, 2H), 1.49-1.33 (m, 2H), 1.29-1.15 (m, |
| | | | | 1H), 1.03-0.89 (m, 1H) |
| I-854 | RP | QP | 858.3 | 10.51 (s, 1H), 8.79 (d, J = 4.8 Hz, 1H), 7.95 (s, 1H), 7.76- |
| | | | | 7.62 (m, 3H), 7.58 (d, J = 2.0 Hz, 1H), 7.41 (dd, J = 8.0, 2.0 |
| | | | | Hz, 1H), 7.16 (t, J= 8.0 Hz, 1H), 6.97 (d, J = 7.6 Hz, 1H), 5.93 |
| | | | | (s, 1H), 5.00-4.73 (m, 1H), 4.17 (t, J = 8.d4 Hz, 2H), 3.85- |
| | | | | 3.50 (m, 6H), 3.46-3.41 (m, 2H), 3.21 (t, J = 8.4 Hz, 2H), |
| | | | | 3.13-2.99 (m, 2H), 2.99-2.85 (m, 5H), 2.85-2.64 (m, 6H), |
| | | | | 2.60-2.52 (m, 1H), 2.48-2.37 (m, 1H), 1.92-1.76 (m, 2H), |
| | | | | 1.32-1.13 (m, 1H), 1.04-0.87 (m, 1H) |

^{a}The coupling was run at 80-100° C. under standard conditions for 1-2 h. Purification was run under a variety of typical methods including prep-HPLC, reverse phase chromatography, and silica gel chromatography.

### Example 7. TYK2 MSD Degradation

Degradation of TYK2 in cells was quantitatively measured using Meso Scale Discovery (MSD) technology. OCI-LY10 cells were seeded in 96-well plates with a density of 0.4 million cells per well in 100 μl fresh media. Compounds were then added to the assay plates with a final top concentration of 1 to 10 μM in a 1:3 dilution series with total of 9 doses. The assay plates were then incubated for 4 to 24 hours at 37° C. under 500 CO₂. The assay plates were then centrifuged for 5 minutes, and the cell pellets were treated with 100 μl/well RIPA lysis buffer (Boston Bioproducts, BP-115D) with protease and phosphatase inhibitors (Roche, 05892791001 and 04906837001). To prepare MSD assay plates (MSD, L15XA), the plates were coated with the capture antibody (Santa Cruz Biotechnology, SC-5271) in PBS, at 40 μl/well. The plates were then incubated overnight at 4° C., washed 3 times with 150 μl/well TBST buffer (Cell Signaling Technology, 9997S) and blocked with 150 μl/well blocking buffer (MSD, R93BA-4). Cell lysates were then added to MSD assay plates and the plates were incubated at room temperature for 1 hour. The plates were then washed 3 times with 150 μl/well TBST buffer and 25 μl/well primary detection antibody (Cell Signaling Technology, #9312). The assay plates were then incubated at room temperature for 1 hour, washed 3 times with 150 μl/well TBST buffer, and 25 μl/well secondary detection antibody, SULFO-TAG anti-rabbit antibody (MSD, R32AB-1) were added. The assay plates were then incubated at room temperature for 1 hour, washed 3 times with 150 μl/well TB ST buffer, and 150 μl/well MSD reading buffer (MSD, R92TC-2) were added. The plates were then analyzed by an MSD reader. The data was analyzed by GraphPad Prism and the dose dependent TYK2 degradation was fit using the four-parameter inhibitor vs. response nonlinear regression.

TYK2 MSD degradation results in OCI-LY10 cells for compounds of the invention are presented in Table 7. The letter codes for TYK2 DC₅₀ include: A (<1 nM); B (i to <10 nM); C (10 to 100 nM); D (>100 nM); and E (not tested). The letter codes for TYK2 Dmax % include: A (>90%); B (90 to >75%); C (75 to 50%); D (<50%); and E (not tested).

TABLE 7
TYK2 DC₅₀ in OCI-LY10 cells

| | TYK2 MSD | TYK2 MSD |
|---|---|---|
| | OCI-LY10 | OCI-LY10 |
| I-# | Rel-DC₅₀ | Dmax % |
| I-1 | A | B |
| 1-2 | D | D |
| I-3 | D | D |
| I-4 | D | D |
| I-5 | B | C |
| I-6 | E | E |
| I-7 | D | D |
| 1-8 | D | D |
| I-9 | D | D |
| I-10 | A | C |
| I-11 | A | C |
| I-12 | A | C |
| I-13 | B | C |
| I-14 | B | C |
| I-15 | A | C |
| I-16 | B | B |
| I-17 | B | B |
| I-18 | A | C |
| I-19 | B | B |
| I-20 | A | C |
| I-21 | B | D |
| I-22 | D | D |
| I-23 | D | D |
| I-24 | D | D |
| I-25 | D | D |
| I-26 | D | D |
| I-27 | A | C |
| I-28 | A | B |
| I-29 | B | B |
| I-30 | B | B |
| I-31 | B | D |
| I-32 | B | C |
| I-33 | B | C |
| I-34 | D | D |
| I-35 | D | D |
| I-36 | A | C |
| I-37 | B | C |
| I-38 | A | D |
| I-39 | B | D |
| I-40 | D | D |
| I-41 | D | D |
| I-42 | D | D |
| I-43 | A | C |
| I-44 | B | C |
| I-45 | A | C |
| I-46 | A | C |
| I-47 | B | B |
| I-48 | A | B |
| I-49 | C | C |
| I-50 | A | C |
| I-51 | D | D |
| I-52 | D | D |
| I-53 | D | D |
| I-54 | B | C |
| I-55 | D | D |
| I-56 | B | C |
| I-57 | B | B |
| I-58 | D | D |
| I-59 | A | D |
| I-60 | B | C |
| I-61 | B | B |
| I-62 | A | B |
| I-63 | A | A |
| I-64 | A | A |
| I-65 | D | D |
| I-66 | D | D |
| I-67 | A | A |
| I-68 | A | B |
| I-69 | B | B |
| I-70 | B | C |
| I-71 | A | B |
| I-72 | A | B |
| I-73 | B | C |
| I-74 | B | D |
| I-75 | B | C |
| I-76 | A | B |
| I-77 | B | D |
| I-78 | B | D |
| I-79 | B | D |
| I-80 | B | B |
| I-81 | A | B |
| I-82 | A | B |
| I-83 | B | D |
| I-84 | B | C |
| I-85 | A | B |
| I-86 | B | C |
| I-87 | B | D |
| I-88 | A | B |
| I-89 | A | C |
| I-90 | B | D |
| I-91 | A | B |
| I-92 | C | C |
| I-93 | B | D |
| I-94 | D | D |
| I-95 | D | D |
| I-96 | B | D |
| I-97 | E | E |
| I-98 | E | E |
| I-99 | E | E |
| I-100 | E | E |
| I-101 | E | E |
| I-102 | E | E |
| I-103 | E | E |
| I-104 | E | E |
| I-105 | E | E |
| I-106 | E | E |
| I-107 | E | E |
| I-108 | E | E |
| I-109 | E | E |
| I-110 | E | E |
| I-111 | E | E |
| I-112 | E | E |
| I-113 | E | E |
| I-114 | E | E |
| I-115 | E | E |
| I-116 | E | E |
| I-117 | E | E |
| I-118 | E | E |
| I-119 | E | E |
| I-120 | E | E |
| I-121 | E | E |
| I-122 | E | E |
| I-123 | E | E |
| I-124 | E | E |
| I-125 | E | E |
| I-126 | E | E |
| I-127 | E | E |
| I-128 | E | E |
| I-129 | E | E |
| I-130 | E | E |
| I-131 | E | E |
| I-132 | E | E |
| I-133 | E | E |
| I-134 | E | E |
| I-135 | E | E |
| I-136 | E | E |
| I-137 | E | E |
| I-138 | E | E |
| I-139 | E | E |
| I-140 | E | E |
| I-141 | E | E |
| I-142 | E | E |
| I-143 | E | E |
| I-144 | E | E |
| I-145 | E | E |
| I-146 | E | E |
| I-147 | E | E |
| I-148 | E | E |
| I-149 | E | E |
| I-150 | E | E |
| I-151 | E | E |
| I-152 | E | E |
| I-153 | E | E |
| I-154 | E | E |
| I-155 | E | E |
| I-156 | E | E |
| I-157 | E | E |
| I-158 | E | E |
| I-159 | E | E |
| I-160 | E | E |
| I-161 | E | E |
| I-162 | E | E |
| I-163 | E | E |
| I-164 | E | E |
| I-165 | E | E |
| I-166 | E | E |
| I-167 | E | E |
| I-168 | E | E |
| I-169 | E | E |
| I-170 | E | E |
| I-171 | E | E |
| I-172 | E | E |
| I-173 | E | E |
| I-174 | E | E |
| I-175 | E | E |
| I-176 | E | E |
| I-177 | E | E |
| I-178 | E | E |
| I-179 | E | E |
| I-180 | E | E |
| I-181 | E | E |
| I-182 | E | E |
| I-183 | E | E |
| I-184 | E | E |
| I-185 | E | E |
| I-186 | E | E |
| I-187 | E | E |
| I-188 | E | E |
| I-189 | E | E |
| I-190 | E | E |
| I-191 | E | E |
| I-192 | E | E |
| I-193 | E | E |
| I-194 | E | E |
| I-195 | E | E |
| I-196 | D | D |
| I-197 | D | D |
| I-198 | D | D |
| I-199 | E | E |
| I-200 | E | E |
| I-201 | E | E |
| I-202 | E | E |
| I-203 | E | E |
| I-204 | E | E |
| I-205 | E | E |
| I-206 | E | E |
| I-207 | E | E |
| I-208 | E | E |
| I-209 | E | E |
| I-210 | E | E |
| I-211 | E | E |
| I-212 | E | E |
| I-213 | E | E |
| I-214 | E | E |
| I-215 | E | E |
| I-216 | E | E |
| I-217 | E | E |
| I-218 | E | E |
| I-219 | E | E |
| I-220 | E | E |
| I-221 | E | E |
| I-222 | E | E |
| I-223 | E | E |
| I-224 | E | E |
| I-225 | E | E |
| I-226 | E | E |
| I-227 | E | E |
| I-228 | E | E |
| I-229 | E | E |
| I-230 | E | E |
| I-231 | E | E |
| I-232 | E | E |
| I-233 | E | E |
| I-234 | E | E |
| I-235 | E | E |
| I-236 | E | E |
| I-237 | A | B |
| I-238 | E | E |
| I-239 | E | E |
| I-240 | E | E |
| I-241 | B | C |
| I-242 | B | C |
| I-243 | E | E |
| I-244 | E | E |
| I-245 | D | D |
| I-246 | E | E |
| I-247 | B | B |
| I-248 | B | C |
| I-249 | D | D |
| I-250 | A | B |
| I-251 | B | C |
| I-252 | B | C |
| I-253 | D | D |
| I-254 | B | C |
| I-255 | C | C |
| I-256 | B | D |
| I-257 | E | E |
| I-258 | E | E |
| I-259 | E | E |
| I-260 | E | E |
| I-261 | E | E |
| I-262 | E | E |
| I-263 | D | D |
| I-264 | E | E |
| I-265 | E | E |
| I-266 | E | E |
| I-267 | E | E |
| I-268 | E | E |
| I-269 | E | E |
| I-270 | D | D |
| I-271 | E | E |
| I-272 | D | D |
| I-273 | E | E |
| I-274 | E | E |
| I-275 | E | E |
| I-276 | E | E |
| I-277 | A | B |
| I-278 | E | E |
| I-279 | B | B |
| I-280 | B | B |
| I-281 | B | C |
| I-282 | A | B |
| I-283 | D | D |
| I-284 | D | D |
| I-285 | E | E |
| I-286 | E | E |
| I-287 | D | D |
| I-288 | D | D |
| I-289 | D | D |
| I-290 | E | E |
| I-291 | D | D |
| I-292 | E | E |
| I-293 | E | E |
| I-294 | B | D |
| I-295 | B | D |
| I-296 | A | C |
| I-297 | B | C |
| I-298 | E | E |
| I-299 | E | E |
| I-300 | E | E |
| I-301 | E | E |
| I-302 | A | C |
| I-303 | B | D |
| I-304 | D | D |
| I-305 | D | D |
| I-306 | D | D |
| I-307 | D | D |
| I-308 | E | E |
| I-309 | D | D |
| I-310 | D | D |
| I-311 | D | C |
| I-312 | D | D |
| I-313 | B | C |
| I-314 | B | D |
| I-315 | E | E |
| I-316 | E | E |
| I-317 | E | E |
| I-318 | E | E |
| I-319 | A | B |
| I-320 | E | E |
| I-321 | E | E |
| I-322 | E | E |
| I-323 | E | E |
| I-324 | C | D |
| I-325 | E | E |
| I-326 | E | E |
| I-327 | E | E |
| I-328 | E | E |
| I-329 | E | E |
| I-330 | E | E |
| I-331 | E | E |
| I-332 | E | E |
| I-333 | E | E |
| I-334 | E | E |
| I-335 | E | E |
| I-336 | E | E |
| I-337 | E | E |
| I-338 | E | E |
| I-339 | B | D |
| I-340 | E | E |
| I-341 | A | B |
| I-342 | E | E |
| I-343 | E | E |
| I-344 | E | E |
| I-345 | B | C |
| I-346 | E | E |
| I-347 | A | B |
| I-348 | E | E |
| I-349 | E | E |
| I-350 | E | E |
| I-351 | A | B |
| I-352 | A | B |
| I-353 | D | D |
| I-354 | E | E |
| I-355 | E | E |
| I-356 | E | E |
| I-357 | A | A |
| I-358 | A | A |
| I-359 | A | B |
| I-360 | B | B |
| I-361 | A | B |
| I-362 | D | D |
| I-363 | D | D |
| I-364 | B | C |
| I-365 | B | C |
| I-366 | B | D |
| I-367 | B | C |
| I-368 | D | D |
| I-369 | D | D |
| I-370 | D | D |
| I-371 | B | C |
| I-372 | B | D |
| I-373 | B | D |
| I-374 | B | D |
| I-375 | B | D |
| I-376 | B | B |
| I-377 | D | D |
| I-378 | D | D |
| I-379 | A | D |
| I-380 | A | C |
| I-381 | A | C |
| I-382 | A | B |
| I-383 | A | B |
| I-384 | E | E |
| I-385 | E | E |
| I-386 | E | E |
| I-387 | C | B |
| I-388 | E | E |
| I-389 | B | C |
| I-390 | E | E |
| I-391 | E | E |
| I-392 | E | E |
| I-393 | D | D |
| I-394 | D | D |
| I-395 | D | D |
| I-396 | E | E |
| I-397 | E | E |
| I-398 | E | E |
| I-399 | A | B |
| I-400 | E | E |
| I-401 | E | E |
| I-402 | E | E |
| I-403 | E | E |
| I-404 | E | E |
| I-405 | B | C |
| I-406 | E | E |
| I-407 | E | E |
| I-408 | D | D |
| I-409 | D | D |
| I-410 | D | D |
| I-411 | D | D |
| I-412 | D | D |
| I-413 | E | E |
| I-414 | E | E |
| I-415 | E | E |
| I-416 | E | E |
| I-417 | D | D |
| I-418 | E | E |
| I-419 | E | E |
| I-420 | E | E |
| I-421 | D | D |
| I-422 | E | E |
| I-423 | B | C |
| I-424 | B | D |
| I-425 | D | D |
| I-426 | E | E |
| I-427 | B | D |
| I-428 | E | E |
| I-429 | B | B |
| I-430 | B | D |
| I-431 | B | B |
| I-432 | E | E |
| I-433 | E | E |
| I-434 | D | D |
| I-435 | D | D |
| I-436 | E | E |
| I-437 | B | C |
| I-438 | B | C |
| I-439 | E | E |
| I-440 | C | D |
| I-441 | B | C |
| I-442 | B | C |
| I-443 | C | D |
| I-444 | E | E |
| I-445 | B | B |
| I-446 | B | D |
| I-447 | B | C |
| I-448 | B | D |
| I-449 | E | E |
| I-450 | E | E |
| I-451 | E | E |
| I-452 | E | E |
| I-453 | E | E |
| I-454 | E | E |
| I-455 | E | E |
| I-456 | D | D |
| I-457 | E | E |
| I-458 | B | D |
| I-459 | E | E |
| I-460 | D | D |
| I-461 | B | D |
| I-462 | C | D |
| I-463 | D | D |
| I-464 | E | E |
| I-465 | B | B |
| I-466 | A | A |
| I-467 | B | B |
| I-468 | A | B |
| I-469 | B | B |
| I-470 | A | B |
| I-471 | A | B |
| I-472 | E | E |
| I-473 | A | B |
| I-474 | E | E |
| I-475 | E | E |
| I-476 | E | E |
| I-477 | E | E |
| I-478 | E | E |
| I-479 | E | E |
| I-480 | B | B |
| I-481 | B | D |
| I-482 | B | D |
| I-483 | E | E |
| I-484 | E | E |
| I-485 | B | D |
| I-486 | B | B |
| I-487 | D | D |
| I-488 | C | D |
| I-489 | E | E |
| I-490 | E | E |
| I-491 | E | E |
| I-492 | E | E |
| I-493 | E | E |
| I-494 | E | E |
| I-495 | E | E |
| I-496 | B | B |
| I-497 | E | E |
| I-498 | E | E |
| I-499 | B | C |
| I-500 | D | D |
| I-501 | B | C |
| I-502 | A | A |
| I-503 | E | E |
| I-504 | E | E |
| I-505 | E | E |
| I-506 | E | E |
| I-507 | E | E |
| I-508 | E | E |
| I-509 | E | E |
| I-510 | E | E |
| I-511 | E | E |
| I-512 | E | E |
| I-513 | C | D |
| I-514 | E | E |
| I-515 | E | E |
| I-516 | E | E |
| I-517 | E | E |
| I-518 | E | E |
| I-519 | E | E |
| I-520 | D | D |
| I-521 | E | E |
| I-522 | E | E |
| I-523 | E | E |
| I-524 | E | E |
| I-525 | E | E |
| I-526 | E | E |
| I-527 | E | E |
| I-528 | E | E |
| I-529 | E | E |
| I-530 | E | E |
| I-531 | B | D |
| I-532 | E | E |
| I-533 | E | E |
| I-534 | E | E |
| I-535 | E | E |
| I-536 | E | E |
| I-537 | E | E |
| I-538 | E | E |
| I-539 | E | E |
| I-540 | E | E |
| I-541 | E | E |
| I-542 | E | E |
| I-543 | E | E |
| I-544 | E | E |
| I-545 | E | E |
| I-546 | D | D |
| I-547 | C | C |
| I-548 | B | D |
| I-549 | E | E |
| I-550 | E | E |
| I-551 | E | E |
| I-552 | E | E |
| I-553 | E | E |
| I-554 | E | E |
| I-555 | E | E |
| I-556 | E | E |
| I-557 | E | E |
| I-558 | E | E |
| I-559 | E | E |
| I-560 | E | E |
| I-561 | E | E |
| I-562 | E | E |
| I-563 | E | E |
| I-564 | E | E |
| I-565 | E | E |
| I-566 | E | E |
| I-567 | E | E |
| I-568 | E | E |
| I-569 | E | E |
| I-570 | E | E |
| I-571 | E | E |
| I-572 | E | E |
| I-573 | E | E |
| I-574 | E | E |
| I-575 | E | E |
| I-576 | E | E |
| I-577 | E | E |
| I-578 | E | E |
| I-579 | E | E |
| I-580 | E | E |
| I-581 | E | E |
| I-582 | E | E |
| I-583 | E | E |
| I-584 | E | E |
| I-585 | E | E |
| I-586 | E | E |
| I-587 | E | E |
| I-588 | E | E |
| I-589 | E | E |
| I-590 | E | E |
| I-591 | E | E |
| I-592 | E | E |
| I-593 | E | E |
| I-594 | E | E |
| I-595 | E | E |
| I-596 | E | E |
| I-597 | E | E |
| I-598 | B | D |
| I-599 | E | E |
| I-600 | D | D |
| I-601 | D | D |
| I-602 | E | E |
| I-603 | E | E |
| I-604 | D | C |
| I-605 | E | E |
| I-606 | E | E |
| I-607 | E | E |
| I-608 | E | E |
| I-609 | E | E |
| I-610 | E | E |
| I-611 | E | E |
| I-612 | E | E |
| I-613 | E | E |
| I-614 | E | E |
| I-615 | E | E |
| I-616 | E | E |
| I-617 | B | C |
| I-618 | E | E |
| I-619 | E | E |
| I-620 | E | E |
| I-621 | E | E |
| I-622 | E | E |
| I-623 | E | E |
| I-624 | E | E |
| I-625 | E | E |
| I-626 | E | E |
| I-627 | E | E |
| I-628 | E | E |
| I-629 | E | E |
| I-630 | B | B |
| I-631 | A | B |
| I-632 | E | E |
| I-633 | E | E |
| I-634 | A | A |
| I-635 | E | E |
| I-636 | E | E |
| I-637 | E | E |
| I-638 | E | E |
| I-639 | E | E |
| I-640 | E | E |
| I-641 | E | E |
| I-642 | E | E |
| I-643 | E | E |
| I-644 | E | E |
| I-645 | E | E |
| I-646 | E | E |
| I-647 | A | A |
| I-648 | B | B |
| I-649 | A | B |
| I-650 | E | E |
| I-651 | A | B |
| I-652 | A | B |
| I-653 | B | B |
| I-654 | B | B |
| I-655 | B | B |
| I-656 | A | B |
| I-657 | A | A |
| I-658 | B | B |
| I-659 | A | B |
| I-660 | B | B |
| I-661 | B | B |
| I-662 | A | B |
| I-663 | B | C |
| I-664 | B | C |
| I-665 | B | B |
| I-666 | C | C |
| I-667 | C | C |
| I-668 | B | B |
| I-669 | B | B |
| I-670 | A | B |
| I-671 | C | C |
| I-672 | E | E |
| I-673 | B | C |
| I-674 | A | B |
| I-675 | C | C |
| I-676 | E | E |
| I-677 | E | E |
| I-678 | E | E |
| I-679 | E | E |
| I-680 | C | D |
| I-681 | B | C |
| I-682 | C | D |
| I-683 | B | C |
| I-684 | E | E |
| I-685 | B | C |
| I-686 | B | B |
| I-687 | A | B |
| I-688 | E | E |
| I-689 | E | E |
| I-690 | E | E |
| I-691 | E | E |
| I-692 | E | E |
| I-693 | E | E |
| I-694 | E | E |
| I-695 | E | E |
| I-696 | E | E |
| I-697 | B | B |
| I-698 | A | B |
| I-699 | E | E |
| I-700 | A | B |
| I-701 | A | A |
| I-702 | A | B |
| I-703 | E | E |
| I-704 | E | E |
| I-705 | E | E |
| I-706 | A | B |
| I-707 | B | D |
| I-708 | A | D |
| I-709 | B | D |
| I-710 | B | B |
| I-711 | B | B |
| I-712 | A | B |
| I-713 | B | B |
| I-714 | A | B |
| I-715 | A | B |
| I-716 | A | B |
| I-717 | B | C |
| I-718 | E | E |
| I-719 | B | C |
| I-720 | E | E |
| I-721 | E | E |
| I-722 | E | E |
| I-723 | D | D |
| I-724 | E | E |
| I-725 | E | E |
| I-726 | E | E |
| I-727 | E | E |
| I-728 | E | E |
| I-729 | E | E |
| I-730 | E | E |
| I-731 | E | E |
| I-732 | E | E |
| I-733 | E | E |
| I-734 | E | E |
| I-735 | E | E |
| I-736 | E | E |
| I-737 | E | E |
| I-738 | E | E |
| I-739 | E | E |
| I-740 | E | E |
| I-741 | E | E |
| I-742 | C | C |
| I-743 | B | B |
| I-744 | B | B |
| I-745 | D | D |
| I-746 | B | B |
| I-747 | B | B |
| I-748 | D | D |
| I-749 | B | B |
| I-750 | A | A |
| I-751 | B | B |
| I-752 | B | B |
| I-753 | A | A |
| I-754 | A | A |
| I-755 | C | C |
| I-756 | B | B |
| I-757 | B | B |
| I-758 | B | B |
| I-759 | D | D |
| I-760 | B | B |
| I-761 | B | B |
| I-762 | D | D |
| I-763 | B | B |
| I-764 | B | B |
| I-765 | B | B |
| I-766 | A | A |
| I-767 | D | D |
| I-768 | D | D |
| I-769 | B | B |
| I-770 | B | B |
| I-771 | B | B |
| I-772 | A | A |
| I-773 | D | D |
| I-774 | A | A |
| I-775 | A | A |
| I-776 | B | B |
| I-777 | A | A |
| I-778 | B | B |
| I-779 | D | D |
| I-780 | A | A |
| I-781 | D | D |
| I-782 | A | A |
| I-783 | B | B |
| I-784 | C | C |
| I-785 | B | B |
| I-786 | C | C |
| I-787 | D | D |
| I-788 | B | B |
| I-789 | D | D |
| I-790 | A | A |
| I-791 | A | A |
| I-792 | B | B |
| I-793 | A | A |
| I-794 | B | B |
| I-795 | A | A |
| I-796 | A | A |
| I-797 | A | A |
| I-798 | D | D |
| I-799 | B | B |
| I-800 | D | D |
| I-801 | D | D |
| I-802 | B | B |
| I-803 | A | A |
| I-804 | A | A |
| I-805 | A | A |
| I-806 | D | D |
| I-807 | B | B |
| I-808 | A | A |
| I-809 | A | A |
| I-810 | B | B |
| I-811 | A | A |
| I-812 | D | D |
| I-813 | A | A |
| I-814 | B | B |
| I-815 | B | B |
| I-816 | B | B |
| I-817 | A | A |
| I-818 | B | B |
| I-819 | A | A |
| I-820 | B | B |
| I-821 | A | A |
| I-822 | A | A |
| I-823 | A | A |
| I-824 | B | B |
| I-825 | A | A |
| I-826 | B | B |
| I-827 | D | D |
| I-828 | D | D |
| I-829 | A | A |
| I-830 | B | B |
| I-831 | B | B |
| I-832 | A | A |
| I-833 | B | B |
| I-834 | B | B |
| I-835 | B | B |
| I-836 | B | B |
| I-837 | D | D |
| I-838 | A | A |
| I-839 | A | A |
| I-840 | A | A |
| I-841 | A | A |
| I-842 | A | A |
| I-843 | A | A |
| I-844 | B | B |
| I-845 | A | A |
| I-846 | A | A |
| I-847 | A | A |
| I-848 | A | A |
| I-849 | B | B |
| I-850 | B | B |
| I-851 | C | C |
| I-852 | B | B |
| I-853 | A | A |
| I-854 | A | A |
| I-855 | A | A |
| I-856 | C | C |
| I-857 | A | A |
| I-858 | D | D |
| I-859 | B | B |
| I-860 | D | D |
| I-861 | A | A |
| I-862 | D | D |
| I-863 | B | B |
| I-864 | A | A |
| I-865 | A | A |
| I-866 | C | C |
| I-867 | C | C |
| I-868 | C | C |
| I-869 | A | A |
| I-870 | C | C |
| I-871 | B | B |
| I-872 | B | B |
| I-873 | D | D |
| I-874 | A | A |
| I-875 | B | B |
| I-876 | A | A |
| I-877 | D | D |
| I-878 | B | B |
| I-879 | A | A |
| I-880 | B | B |
| I-881 | B | B |
| I-882 | D | D |
| I-883 | A | A |
| I-884 | C | C |
| I-885 | C | C |
| I-886 | B | B |
| I-887 | B | B |
| I-888 | B | B |
| I-889 | D | D |
| I-890 | B | B |
| I-891 | D | D |
| I-892 | B | B |
| I-893 | D | D |
| I-894 | B | B |
| I-895 | D | D |
| I-896 | D | D |
| I-897 | B | B |
| I-898 | D | D |
| I-899 | D | D |
| I-900 | B | B |
| I-901 | A | A |
| I-902 | B | B |
| I-903 | B | B |
| I-904 | A | A |
| I-905 | D | D |
| I-906 | A | B |
| I-907 | B | B |
| I-908 | B | B |
| I-909 | C | C |
| I-910 | B | B |
| I-911 | B | C |
| I-912 | D | D |
| I-913 | D | D |
| I-914 | D | D |
| I-915 | C | C |
| I-916 | D | D |
| I-917 | D | D |
| I-918 | D | D |
| I-919 | D | D |
| I-920 | D | D |
| I-921 | A | A |
| I-922 | E | E |
| I-923 | E | E |
| I-924 | E | E |
| I-925 | E | E |
| I-926 | E | E |
| I-927 | E | E |
| I-928 | A | A |
| I-929 | A | B |
| I-930 | A | A |
| I-931 | A | A |
| I-932 | B | A |
| I-933 | A | A |
| I-934 | D | C |
| I-935 | A | A |
| I-936 | A | A |
| I-937 | A | B |
| I-938 | A | A |
| I-939 | A | A |
| I-940 | A | A |
| I-941 | C | C |
| I-942 | B | A |
| I-943 | A | A |
| I-944 | A | A |
| I-945 | A | A |
| I-946 | A | A |
| I-947 | D | D |
| I-948 | A | A |
| I-949 | B | B |
| I-950 | A | A |
| I-951 | A | A |
| I-952 | A | A |
| I-953 | A | A |
| I-954 | B | A |
| I-955 | B | A |
| I-956 | B | A |
| I-957 | B | B |
| I-958 | A | A |
| I-959 | A | A |
| I-960 | B | B |
| I-961 | B | D |
| I-962 | A | A |
| I-963 | C | C |
| I-964 | A | B |
| I-965 | A | C |
| I-966 | B | B |
| I-967 | B | B |
| I-968 | A | B |
| I-969 | B | C |
| I-970 | A | A |
| I-971 | A | B |
| I-972 | A | B |
| I-973 | A | B |
| I-974 | B | B |
| I-975 | B | B |
| I-976 | A | B |
| I-977 | B | C |
| I-978 | B | D |
| I-979 | A | A |
| I-980 | B | C |
| I-981 | A | A |
| I-982 | A | B |
| I-983 | A | D |
| I-984 | B | B |
| I-985 | A | A |
| I-986 | B | B |
| I-987 | B | B |
| I-988 | B | B |
| I-989 | A | A |
| I-990 | B | B |
| I-991 | B | B |
| I-992 | A | A |
| I-993 | A | A |
| I-994 | A | A |
| I-995 | A | A |
| I-996 | B | B |
| I-997 | A | A |
| I-998 | C | C |
| I-999 | D | D |
| I-1000 | A | A |
| I-1001 | A | C |
| I-1002 | A | C |
| I-1003 | A | C |
| I-1004 | A | A |
| I-1005 | A | A |
| I-1006 | B | B |
| I-1007 | A | A |
| I-1008 | A | A |
| I-1009 | B | B |
| I-1010 | B | B |
| I-1011 | B | B |
| I-1012 | A | A |
| I-1013 | B | B |
| I-1014 | B | B |
| I-1015 | C | D |
| I-1016 | A | A |
| I-1017 | B | B |
| I-1018 | A | A |
| I-1019 | A | A |
| I-1020 | A | A |
| I-1021 | A | B |
| I-1022 | A | B |
| I-1023 | A | A |
| I-1024 | B | C |
| I-1025 | A | A |
| I-1026 | A | B |
| I-1027 | A | A |
| I-1028 | A | A |
| I-1029 | A | A |
| I-1030 | B | C |
| I-1031 | A | A |
| I-1032 | A | A |
| I-1033 | B | A |
| I-1034 | A | A |
| I-1035 | A | A |
| I-1036 | A | A |
| I-1037 | A | A |
| I-1038 | A | A |
| I-1039 | A | A |
| I-1040 | A | A |
| I-1041 | A | A |
| I-1042 | A | A |
| I-1043 | A | A |
| I-1044 | A | B |
| I-1045 | A | C |
| I-1046 | B | B |
| I-1047 | A | A |
| I-1048 | A | A |
| I-1049 | A | A |
| I-1050 | A | B |
| I-1051 | A | A |
| I-1052 | B | B |
| I-1053 | A | B |
| I-1054 | A | A |
| I-1055 | A | A |
| I-1056 | B | A |
| I-1057 | A | A |
| I-1058 | A | B |
| I-1059 | A | A |
| I-1060 | B | B |
| I-1061 | A | A |
| I-1062 | B | C |
| I-1063 | A | A |
| I-1064 | B | B |
| I-1065 | A | A |
| I-1066 | A | A |
| I-1067 | B | C |
| I-1068 | A | A |
| I-1069 | A | A |
| I-1070 | B | C |
| I-1071 | B | C |
| I-1072 | B | B |
| I-1073 | A | A |
| I-1074 | A | A |
| I-1075 | B | B |
| I-1076 | B | B |
| I-1077 | A | B |
| I-1078 | A | A |
| I-1079 | A | A |
| I-1080 | B | B |
| I-1081 | A | A |
| I-1082 | A | B |
| I-1083 | B | B |
| I-1084 | A | A |
| I-1085 | A | B |
| I-1086 | A | B |
| I-1087 | A | A |
| I-1088 | B | B |
| I-1089 | A | A |
| I-1090 | D | D |
| I-1091 | A | B |
| I-1092 | D | D |
| I-1093 | A | B |
| I-1094 | D | D |
| I-1095 | B | B |
| I-1096 | A | A |
| I-1097 | A | A |
| I-1098 | D | D |
| I-1099 | B | B |
| I-1100 | B | B |
| I-1101 | D | D |
| I-1102 | B | A |
| I-1103 | C | D |
| I-1104 | B | A |
| I-1105 | A | A |
| I-1106 | B | A |
| I-1107 | B | A |
| I-1108 | D | D |
| I-1109 | D | D |
| I-1110 | D | D |
| I-1111 | B | D |
| I-1112 | D | D |
| I-1113 | A | B |
| I-1114 | A | A |
| I-1115 | D | D |
| I-1116 | B | B |
| I-1117 | D | D |
| I-1118 | C | C |
| I-1119 | B | D |
| I-1120 | A | D |
| I-1121 | A | A |
| I-1122 | A | A |
| I-1123 | B | A |
| I-1124 | A | A |
| I-1125 | A | A |
| I-1126 | E | E |
| I-1127 | A | A |
| I-1128 | A | A |
| I-1129 | E | E |
| I-1130 | B | B |
| I-1131 | E | E |
| I-1132 | E | E |
| I-1133 | A | A |
| I-1134 | A | A |
| I-1135 | A | A |
| I-1137 | A | A |
| I-1138 | A | B |
| I-1139 | A | A |
| I-1140 | A | A |
| I-1141 | A | A |
| I-1142 | A | A |
| I-1143 | A | A |
| I-1144 | A | B |
| I-1145 | A | A |
| I-1146 | A | A |
| I-1147 | A | A |
| I-1148 | A | B |
| I-1149 | A | A |
| I-1150 | A | A |
| I-1151 | A | A |
| I-1152 | E | E |
| I-1153 | B | B |
| I-1154 | A | A |
| I-1155 | A | B |
| I-1156 | A | B |
| I-1157 | A | A |
| I-1158 | A | A |
| I-1159 | A | A |
| I-1160 | E | E |
| I-1161 | A | B |
| I-1162 | E | E |
| I-1163 | E | E |
| I-1164 | E | E |
| I-1165 | A | A |
| I-1166 | E | E |
| I-1167 | E | E |
| I-1168 | A | A |
| I-1169 | A | A |
| I-1170 | A | A |
| I-1171 | A | A |
| I-1172 | E | E |
| I-1173 | E | E |
| I-1174 | E | E |
| I-1175 | E | E |
| I-1176 | E | E |
| I-1177 | A | A |
| I-1178 | A | B |
| I-1179 | A | B |
| I-1180 | A | A |
| I-1181 | A | A |
| I-1182 | A | B |
| I-1183 | A | A |
| I-1184 | A | B |
| I-1185 | B | B |
| I-1186 | B | A |
| I-1187 | A | A |
| I-1188 | E | E |
| I-1189 | E | E |
| I-1190 | A | A |
| I-1191 | A | B |
| I-1192 | A | B |
| I-1193 | A | A |
| I-1194 | A | A |
| I-1195 | E | E |
| I-1196 | E | E |
| I-1197 | A | B |
| I-1198 | A | B |
| I-1199 | E | E |
| I-1200 | A | A |
| I-1201 | A | A |
| I-1202 | A | C |
| I-1203 | A | B |
| I-1204 | B | B |
| I-1205 | A | A |
| I-1206 | E | E |
| I-1207 | A | A |
| I-1208 | A | B |
| I-1209 | A | B |
| I-1210 | A | B |
| I-1211 | A | A |
| I-1212 | A | B |
| I-1213 | A | A |
| I-1214 | E | E |
| I-1215 | E | E |
| I-1216 | B | B |
| I-1217 | B | C |
| I-1218 | E | E |
| I-1219 | E | E |
| I-1220 | A | A |
| I-1221 | A | A |
| I-1222 | A | A |
| I-1223 | A | A |
| I-1224 | A | A |
| I-1225 | E | E |
| I-1226 | A | A |
| I-1227 | A | A |
| I-1228 | E | E |
| I-1229 | A | A |
| I-1230 | A | A |
| I-1231 | A | A |
| I-1232 | A | A |
| I-1233 | D | D |
| I-1234 | A | B |
| I-1235 | B | B |
| I-1236 | E | E |
| I-1237 | A | B |
| I-1238 | A | A |
| I-1239 | E | E |
| I-1240 | A | B |
| I-1241 | E | E |
| I-1242 | A | B |
| I-1243 | A | A |
| I-1244 | A | B |
| I-1245 | A | A |
| I-1246 | A | B |
| I-1247 | E | E |
| I-1248 | A | B |
| I-1249 | B | A |
| I-1250 | A | A |
| I-1251 | A | A |
| I-1252 | A | A |
| I-1253 | A | A |
| I-1254 | C | B |
| I-1255 | E | E |
| I-1256 | E | E |
| I-1257 | A | B |
| I-1258 | A | A |

### Example 8. In Vivo Experiments

TYK2 degraders of this invention were dosed in rats. Male SD rats were orally and intravenously administered certain compounds of the invention as a single dose. Oral and intravenous doses ranged from 2 to 10 mpk and compound level was measured in plasma 0, 0.0833, 0.25, 0.5, 1, 2, 4, 8, and 24 hours after dosing.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

LENGTHY TABLES
The patent application contains a lengthy table section. A copy of the table is available in electronic form from the USPTO web site (https://seqdata.uspto.gov/docdetail?docId=US20260125400A1). An electronic copy of the table will also be available from the USPTO upon request and payment of the fee set forth in 37 CFR 1.19(b)(3).

1. A compound of formula I-a′: 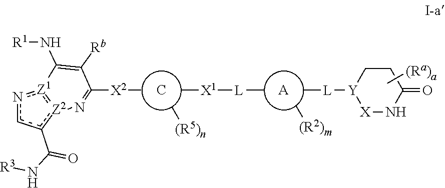
or a pharmaceutically acceptable salt thereof, wherein:
X is a bivalent moiety selected from —CH₂— or —C(O)—;
X¹ is a covalent bond, 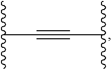 [SMILES: *C#C*]
or an optionally substituted ring selected from 4-6 membered saturated carbocyclylenyl or heterocyclylenyl having 1-2 heteroatoms independently selected from nitrogen, oxygen, and sulfur, phenylenyl, 5-6 membered heteroarylenyl having 1-2 heteroatoms independently selected from nitrogen, oxygen, and sulfur, and a 5-8 membered saturated bridged bicyclic heterocyclylenyl having 1-2 heteroatoms independently selected from nitrogen, oxygen, and sulfur;
X² is a covalent bond, —O—, or —NR—;
Y is N or CH;
Y¹ is a covalent bond, —O—, —S—, —NR—, or —C(O)NR—;
Z¹ and Z² are independently N or C, where one of Z and Z² is N and the other of Z¹ and Z² is C;
each is independently a single or double bond;
Ring A is a ring selected from phenylenyl, pyridinylenyl, 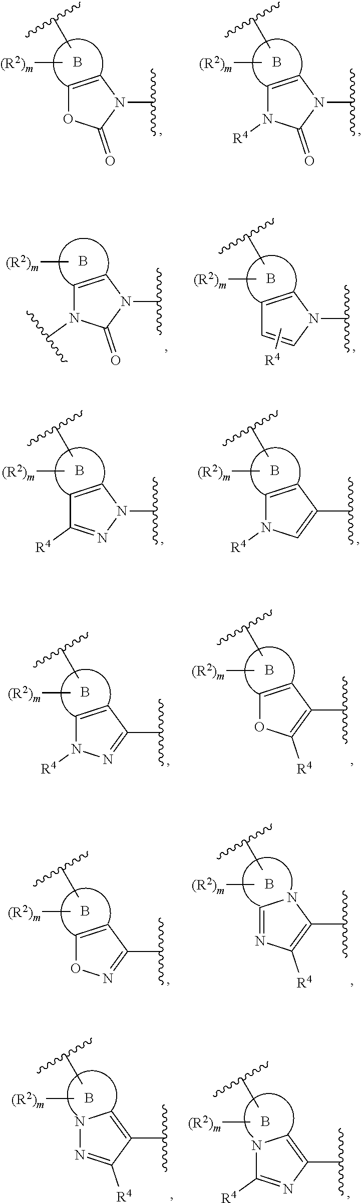 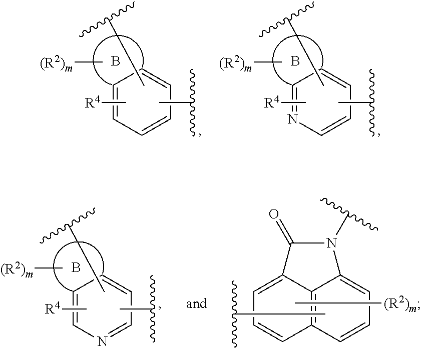
Ring B is a fused ring selected from benzo, a 5-6 membered saturated or partially unsaturated heterocyclyl having 1-3 heteroatoms independently selected from nitrogen, oxygen, and sulfur, or a 5-6 membered heteroaryl containing 1-4 heteroatoms independently selected from nitrogen, oxygen, and sulfur;
Ring C is phenylenyl, pyridylenyl, a 9-10 membered saturated or partially unsaturated bicyclic heterocyclylenyl having 1-3 heteroatoms independently selected from nitrogen, oxygen, and sulfur, 8-10 membered bicyclic heteroarylenyl having 1-3 heteroatoms independently selected from nitrogen, oxygen, and sulfur, 12-15 membered saturated or partially unsaturated tricyclic spirocyclic heterocyclylenyl having 1-3 heteroatoms independently selected from nitrogen, oxygen;
R^{a} is hydrogen, halogen, —CN, —OR, oxo, C₁₋₆alkyl, C₁₋₆haloalkyl, C₃₋₆cycloalkyl, or:
two R^{a} on the same carbon atom or adjacent carbon atoms connect to form a 3-6 membered saturated carbocyclic or heterocyclic ring having 1-2 heteroatoms selected from nitrogen, oxygen, and sulfur;
a is 0, 1, or 2;
R^{b} is hydrogen, or:
R^{b} connects with the nitrogen where R¹ is attached to form an optionally substituted 5-6 membered partially unsaturated or aromatic heterocyclic ring having 0-2 heteroatoms in addition to the nitrogen atom where R¹ is attached selected from nitrogen, oxygen, and sulfur;
R¹ is hydrogen, C₁₋₆ alkyl, C₁₋₆ haloalkyl, or C₃₋₆ cycloalkyl;
each R² and R⁵ is independently hydrogen, halogen, —CN, —NO₂, —OR, oxo, —SR, —NR₂, —SiR₃, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, or R^{A};
m and n are independently 0, 1, 2, 3, or 4;
R³ is hydrogen, or a C₁₋₆ alkyl or 3-9 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur optionally substituted with 1-2 substituents selected from halogen, —CN, C₁₋₆ alkyl, C₁₋₆ haloalkyl, —OR, —CH₂OR, or a 5-membered heteroaryl having 1-3 heteroatoms independently selected from nitrogen, oxygen, and sulfur, or:
R³ and R⁵ are joined by a bivalent, saturated or partially unsaturated, straight or branched C₃₋₆ hydrocarbon chain, wherein 0-2 methylene units of the chain are independently replaced by —CR₂—, —CRF—, —CF₂—, —O—, —NR—, —C(O)—;
R⁴ is hydrogen, halogen, C₁₋₆ alkyl, C₁₋₆ haloalkyl, C₃₋₆ cycloalkyl, —OC₁₋₆ alkyl, or —OC₁₋₆ haloalkyl;
L is a covalent bond, —CR₂—, —CRF—, —CF₂—, —O—, —NR—, —C(O)—, or a bivalent, saturated or partially unsaturated, straight or branched C₁₋₁₀ hydrocarbon chain, wherein 0-6 methylene units of L are independently replaced by -Cy-, —CR₂—, —CRF—, —CF₂—, —CR(OR)—, —O—, —NR—, —C(O)—;
each -Cy- is independently an optionally substituted bivalent ring selected from phenylenyl, 8-10 membered bicyclic arylenyl, 4-7 membered saturated or partially unsaturated carbocyclylenyl, 6-11 membered saturated or partially unsaturated spirocyclic or bridged bicyclic carbocyclylenyl, 8-10 membered bicyclic saturated or partially unsaturated carbocyclylenyl, 4-7 membered saturated or partially unsaturated heterocyclylenyl having 1-2 heteroatoms independently selected from nitrogen, oxygen, and sulfur, 6-11 membered saturated or partially unsaturated spirocyclic or bridged bicyclic heterocyclylenyl having 1-2 heteroatoms independently selected from nitrogen, oxygen, and sulfur, 8-10 membered bicyclic saturated or partially unsaturated heterocyclylenyl having 1-2 heteroatoms independently selected from nitrogen, oxygen, and sulfur, 5-6 membered heteroarylenyl having 1-4 heteroatoms independently selected from nitrogen, oxygen, and sulfur, and 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur; and
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

2. A compound of formula I-a: 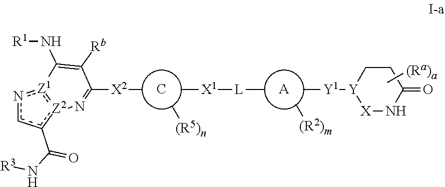
or a pharmaceutically acceptable salt thereof, wherein:
X is a bivalent moiety selected from —CH₂— or —C(O)—;
X¹ is a covalent bond, 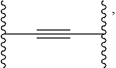 [SMILES: *C#C*]
or an optionally substituted ring selected from 4-6 membered saturated carbocyclylenyl or heterocyclylenyl having 1-2 heteroatoms independently selected from nitrogen, oxygen, and sulfur, phenylenyl, 5-6 membered heteroarylenyl having 1-2 heteroatoms independently selected from nitrogen, oxygen, and sulfur, and a 5-8 membered saturated bridged bicyclic heterocyclylenyl having 1-2 heteroatoms independently selected from nitrogen, oxygen, and sulfur;
X² is a covalent bond, —O—, or —NR—;
Y is N or CH;
Y¹ is a covalent bond, —O—, —S—, —NR—, or —C(O)NR—;
Z¹ and Z² are independently N or C, where one of Z and Z² is N and the other of Z¹ and Z² is C;
Ring A is a ring selected from phenylenyl, pyridinylenyl, 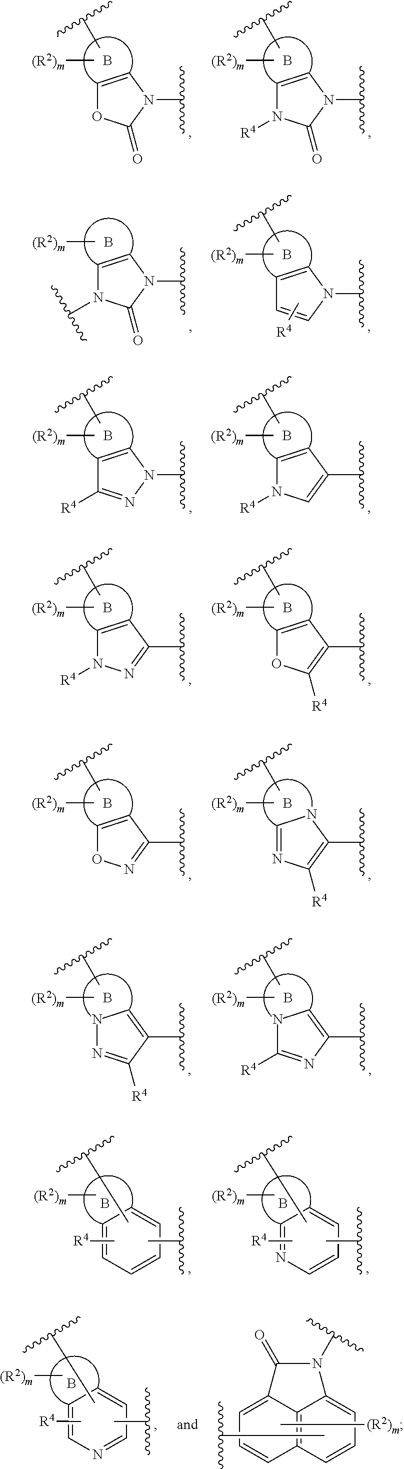
Ring B is a fused ring selected from benzo, a 5-6 membered saturated or partially unsaturated heterocyclyl having 1-3 heteroatoms independently selected from nitrogen, oxygen, and sulfur, or a 5-6 membered heteroaryl containing 1-4 heteroatoms independently selected from nitrogen, oxygen, and sulfur;
Ring C is phenylenyl, pyridylenyl, a 9-10 membered saturated or partially unsaturated bicyclic heterocyclylenyl having 1-3 heteroatoms independently selected from nitrogen, oxygen, and sulfur, 8-10 membered bicyclic heteroarylenyl having 1-3 heteroatoms independently selected from nitrogen, oxygen, and sulfur, 12-15 membered saturated or partially unsaturated tricyclic spirocyclic heterocyclylenyl having 1-3 heteroatoms independently selected from nitrogen, oxygen;
R^{a} is hydrogen, halogen, —CN, —OR, oxo, C₁₋₆alkyl, C₁₋₆haloalkyl, C₃₋₆cycloalkyl, or:
two R^{a} on the same carbon atom or adjacent carbon atoms connect to form a 3-6 membered saturated carbocyclic or heterocyclic ring having 1-2 heteroatoms selected from nitrogen, oxygen, and sulfur;
a is 0, 1, or 2;
R^{b} is hydrogen, or:
R^{b} connects with the nitrogen where R¹ is attached to form an optionally substituted 5-6 membered partially unsaturated or aromatic heterocyclic ring having 0-2 heteroatoms in addition to the nitrogen atom where R¹ is attached selected from nitrogen, oxygen, and sulfur;
R¹ is hydrogen, C₁₋₆ alkyl, C₁₋₆ haloalkyl, or C₃₋₆ cycloalkyl;
each R² and R⁵ is independently hydrogen, halogen, —CN, —NO₂, —OR, oxo, —SR, —NR₂, —SiR₃, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, or R^{A};
m and n are independently 0, 1, 2, 3, or 4;
R³ is hydrogen, or a C₁₋₆ alkyl or C₃₋₆ cycloalkyl optionally substituted with 1-2 substituents selected from halogen, —CN, C₁₋₆alkyl, C₁₋₆haloalkyl, and —OR, or:
R³ and R⁵ are joined by a bivalent, saturated or partially unsaturated, straight or branched C3.6 hydrocarbon chain, wherein 0-2 methylene units of the chain are independently replaced by —CR₂—, —CRF—, —CF₂—, —O—, —NR—, —C(O)—;
R⁴ is hydrogen, halogen, C₁₋₆ alkyl, C₁₋₆ haloalkyl, C₃₋₆ cycloalkyl, —OC₁₋₆ alkyl, or —OC₁₋₆ haloalkyl;
L is a covalent bond, —CR₂—, —CRF—, —CF₂—, —O—, —NR—, —C(O)—, or a bivalent, saturated or partially unsaturated, straight or branched C₁₋₁₀ hydrocarbon chain, wherein 0-6 methylene units of L are independently replaced by -Cy-, —CR₂—, —CRF—, —CF₂—, —CR(OR)—, —O—, —NR—, —C(O)—;
each -Cy- is independently an optionally substituted bivalent ring selected from phenylenyl, 8-10 membered bicyclic arylenyl, 4-7 membered saturated or partially unsaturated carbocyclylenyl, 6-11 membered saturated or partially unsaturated spirocyclic or bridged bicyclic carbocyclylenyl, 8-10 membered bicyclic saturated or partially unsaturated carbocyclylenyl, 4-7 membered saturated or partially unsaturated heterocyclylenyl having 1-2 heteroatoms independently selected from nitrogen, oxygen, and sulfur, 6-11 membered saturated or partially unsaturated spirocyclic or bridged bicyclic heterocyclylenyl having 1-2 heteroatoms independently selected from nitrogen, oxygen, and sulfur, 8-10 membered bicyclic saturated or partially unsaturated heterocyclylenyl having 1-2 heteroatoms independently selected from nitrogen, oxygen, and sulfur, 5-6 membered heteroarylenyl having 1-4 heteroatoms independently selected from nitrogen, oxygen, and sulfur, and 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur; and
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

3. The compound of claim 1 or claim 2, wherein X² is a covalent bond.

4. The compound of any one of claims 1-3, wherein Ring C is phenylenyl, pyridylenyl, a 9-membered partially unsaturated bicyclic heterocyclylenyl having 1-3 heteroatoms independently selected from nitrogen, oxygen, and sulfur, 9-membered bicyclic heteroarylenyl having 1-3 heteroatoms independently selected from nitrogen, oxygen, and sulfur, or a 14-membered partially unsaturated tricyclic spirocyclic heterocyclylenyl having 1-3 heteroatoms independently selected from nitrogen, oxygen

5. The compound of any one of claims 1-4, wherein Ring C is

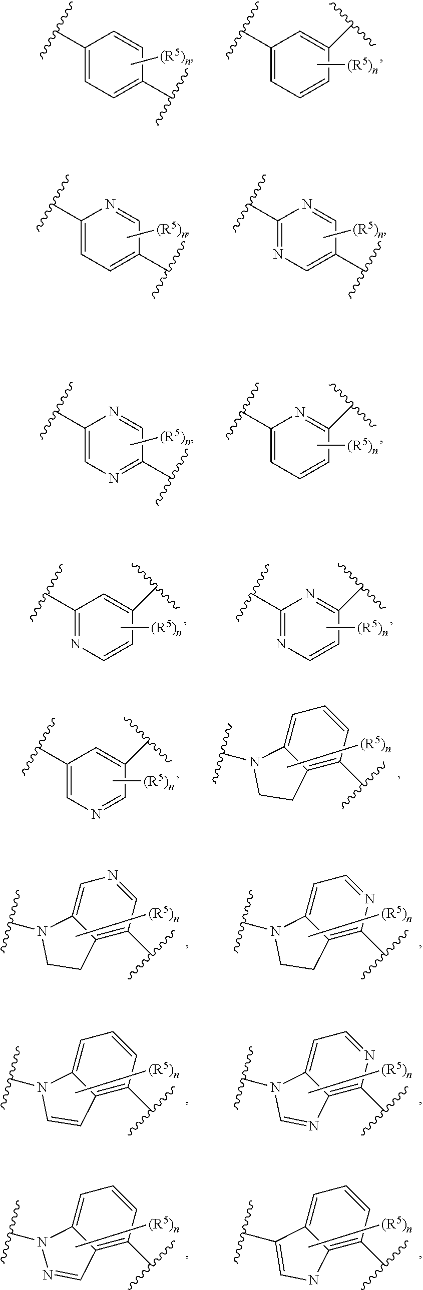

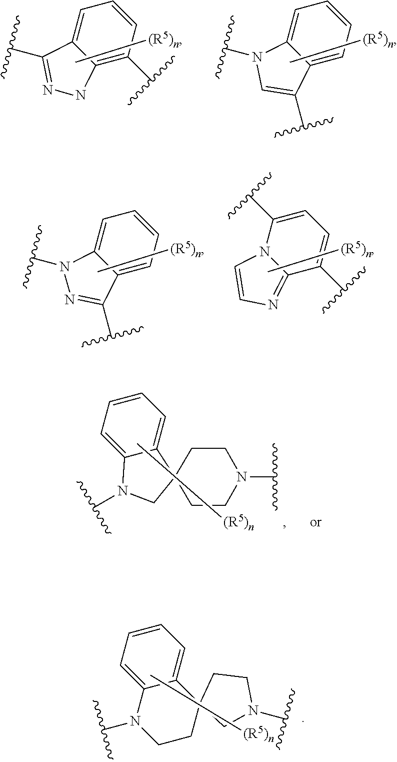

6. The compound of any one of claims 1-5 wherein X¹ is a covalent bond, 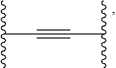 [SMILES: *C#C*]
or an optionally substituted ring selected from 4-6 membered saturated carbocyclylenyl or heterocyclylenyl having 1-2 heteroatoms independently selected from nitrogen, oxygen, and sulfur, phenylenyl, 6-membered heteroarylenyl having 1-2 heteroatoms independently selected from nitrogen, oxygen, and sulfur, and a 8-membered saturated bridged bicyclic heterocyclylenyl having 1-2 heteroatoms independently selected from nitrogen, oxygen, and sulfur.

7. The compound of any one of claims 1-6, wherein X¹ is a covalent bond,

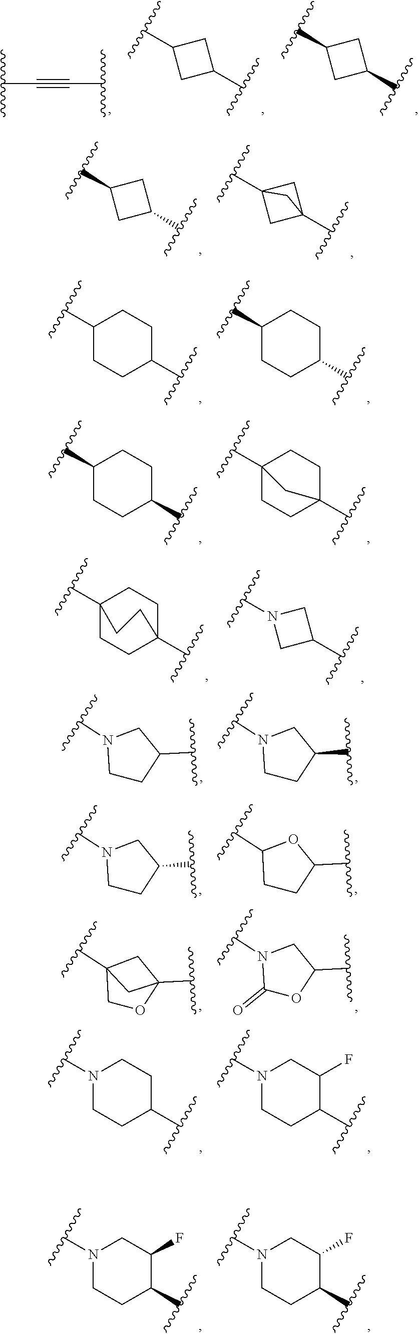

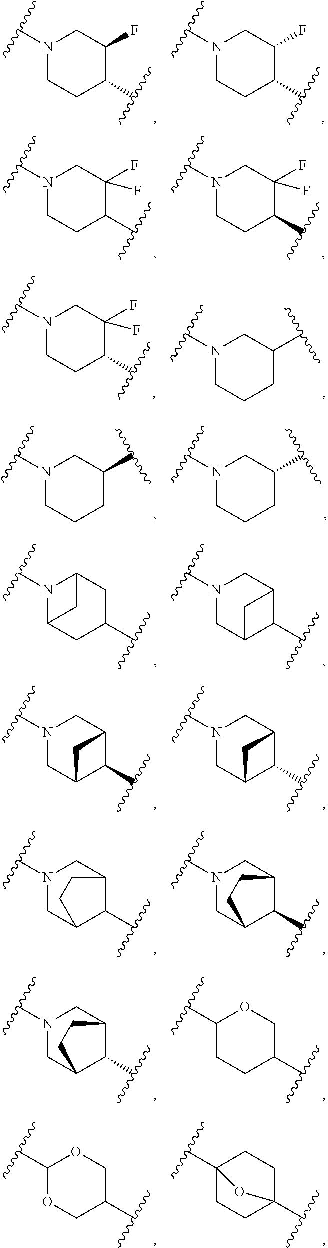

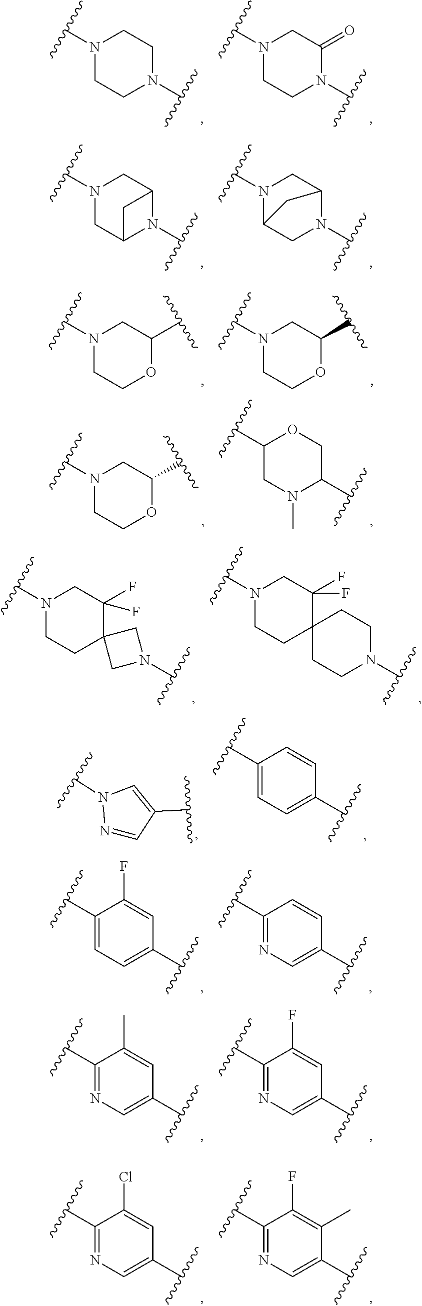

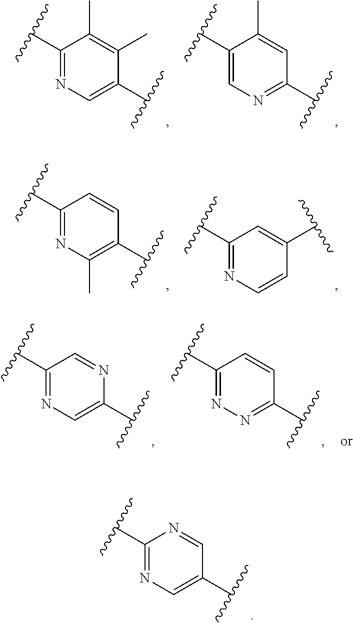

8. The compound of any one of claims 1-7, wherein the compound is of any one of the following formulae: 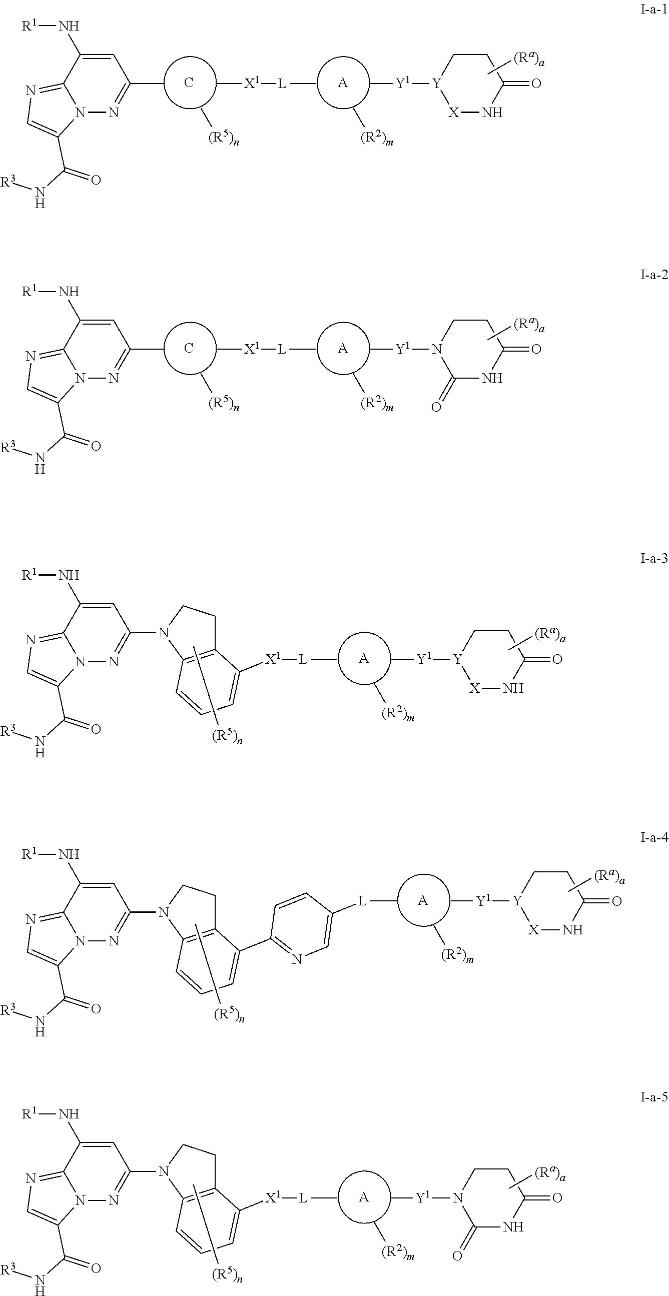 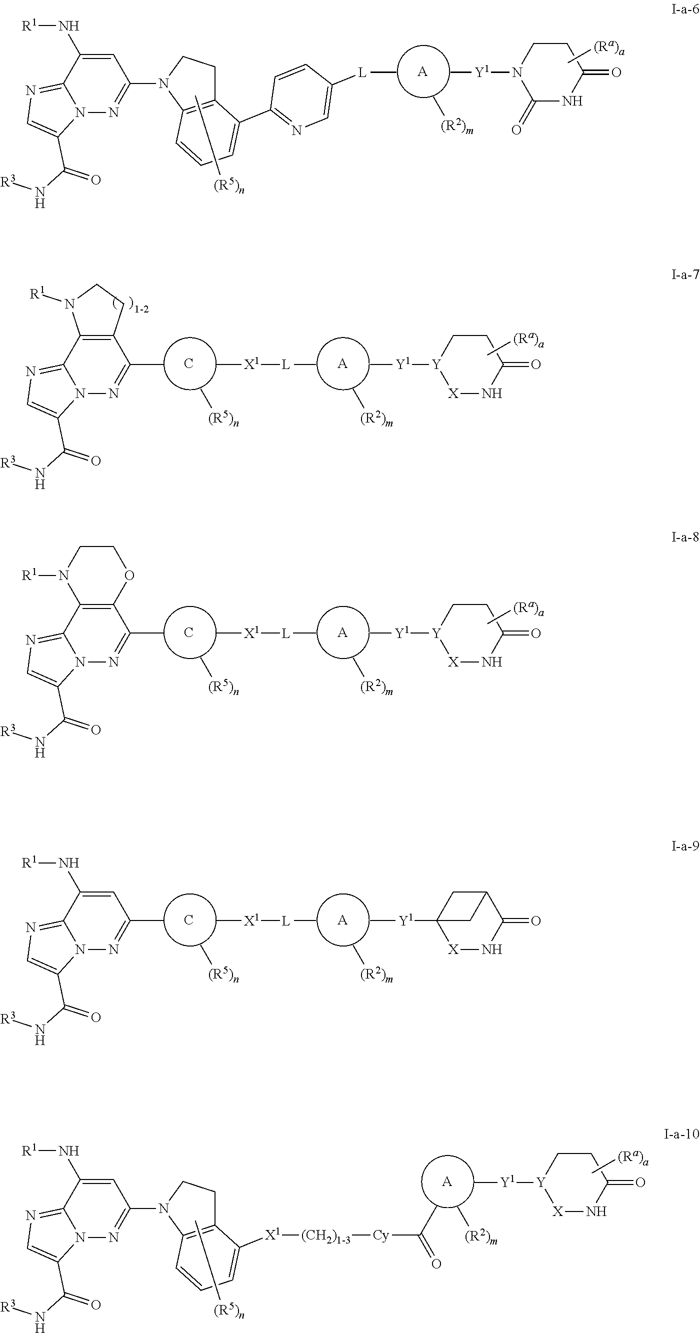 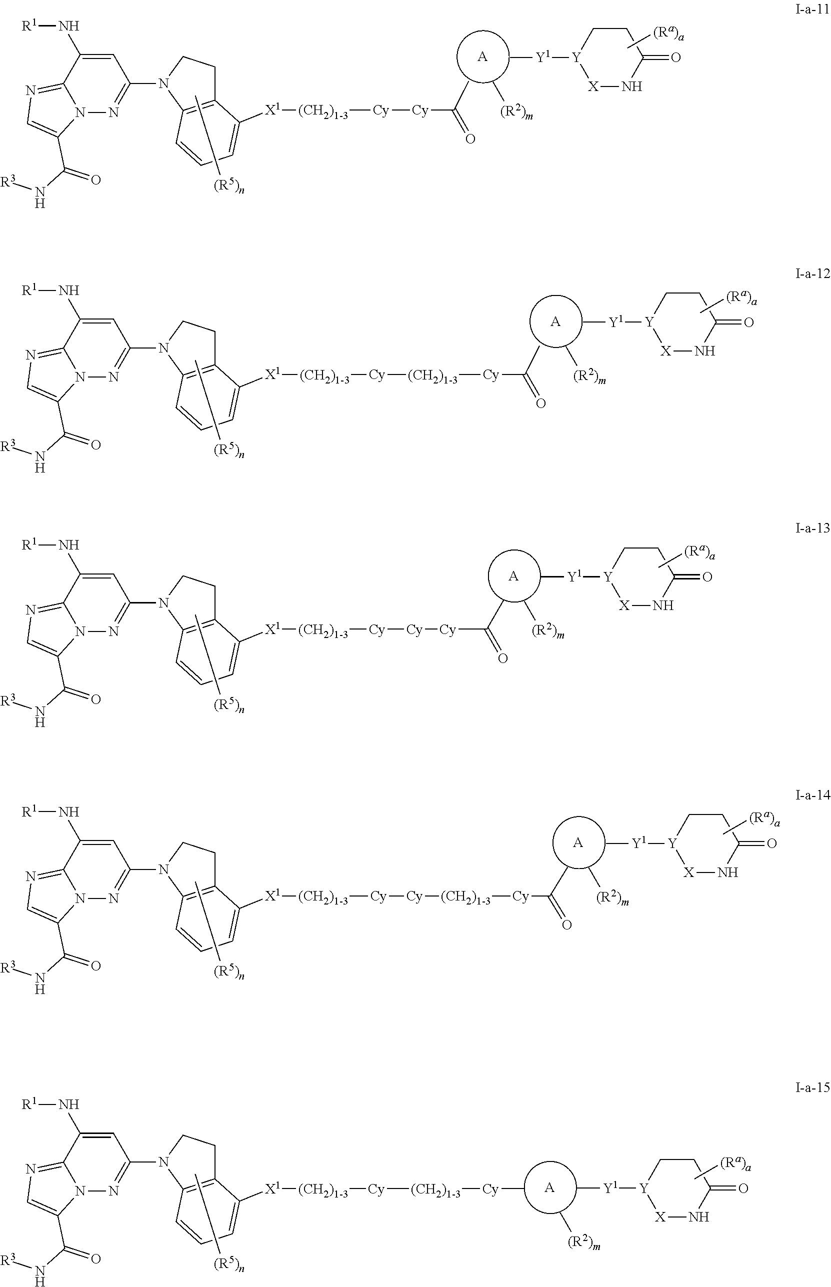 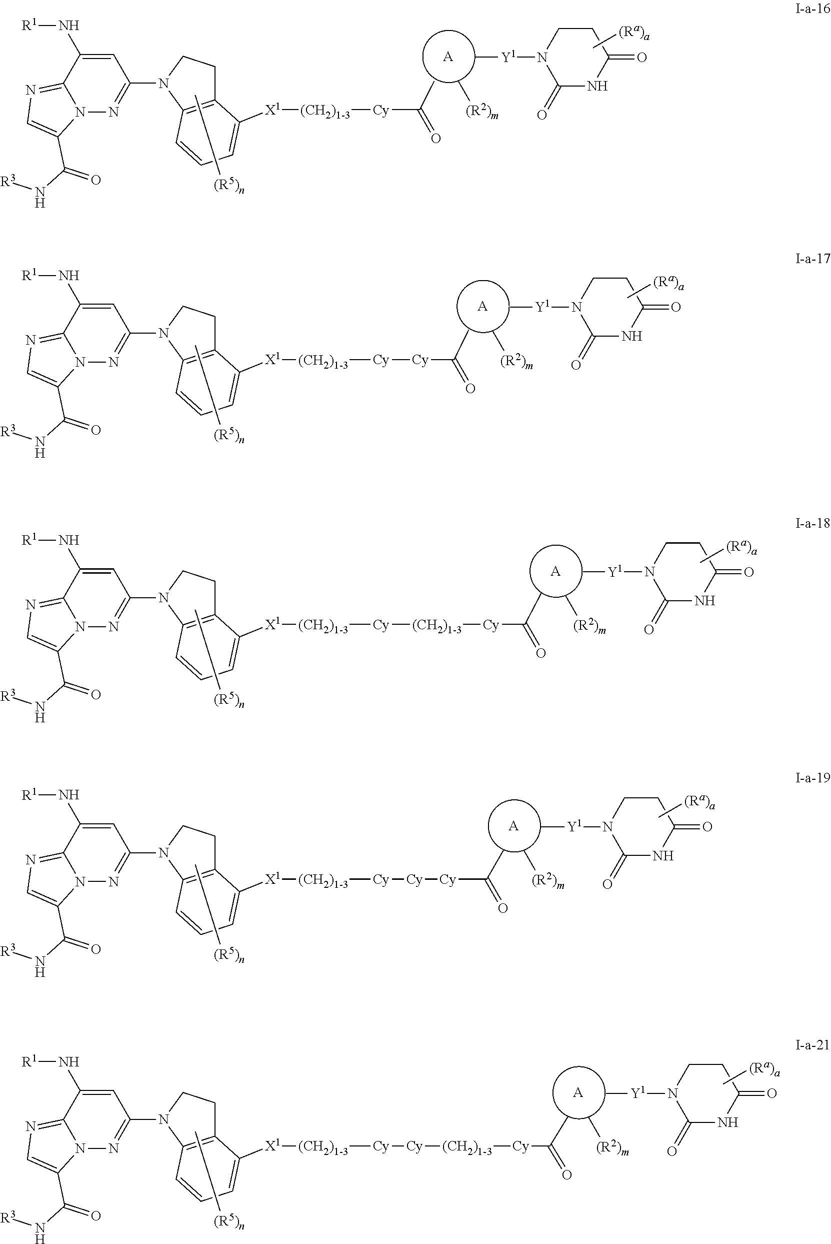 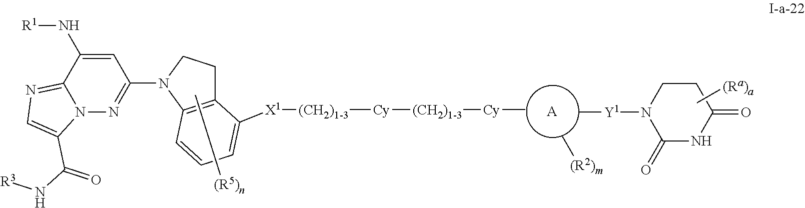
or a pharmaceutically acceptable salt thereof

9. The compound of any one of claims 1-8, wherein R¹ is hydrogen or C₁₋₆ alkyl.

10. The compound of any one of claims 1-9, wherein R³ is hydrogen, methyl, ethyl, isopropyl,

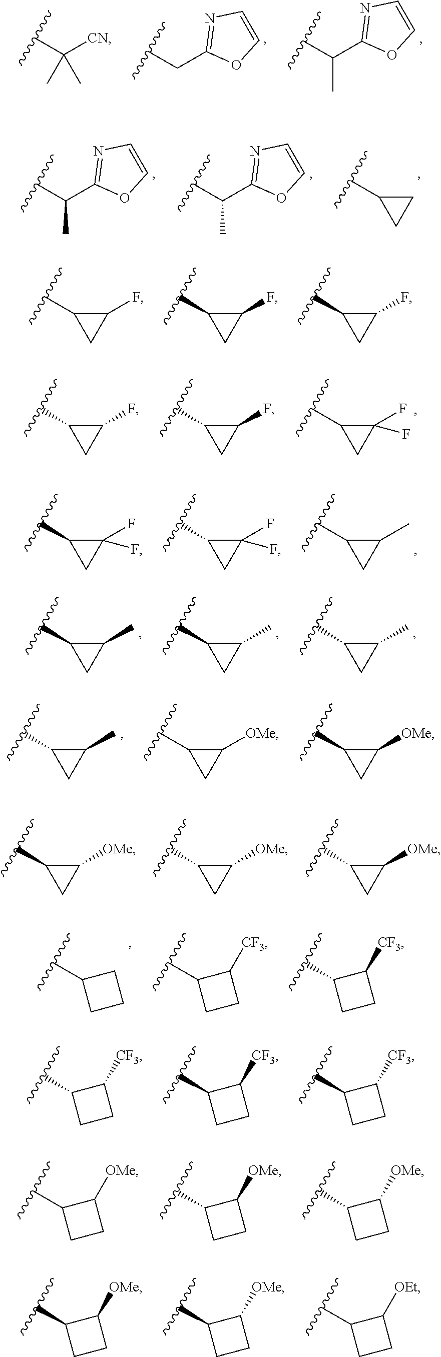

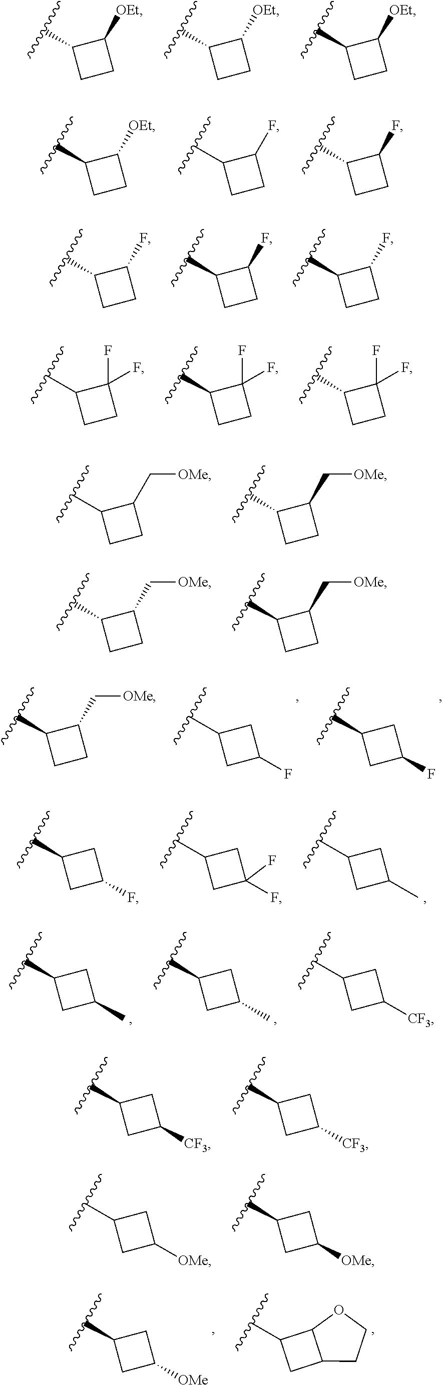

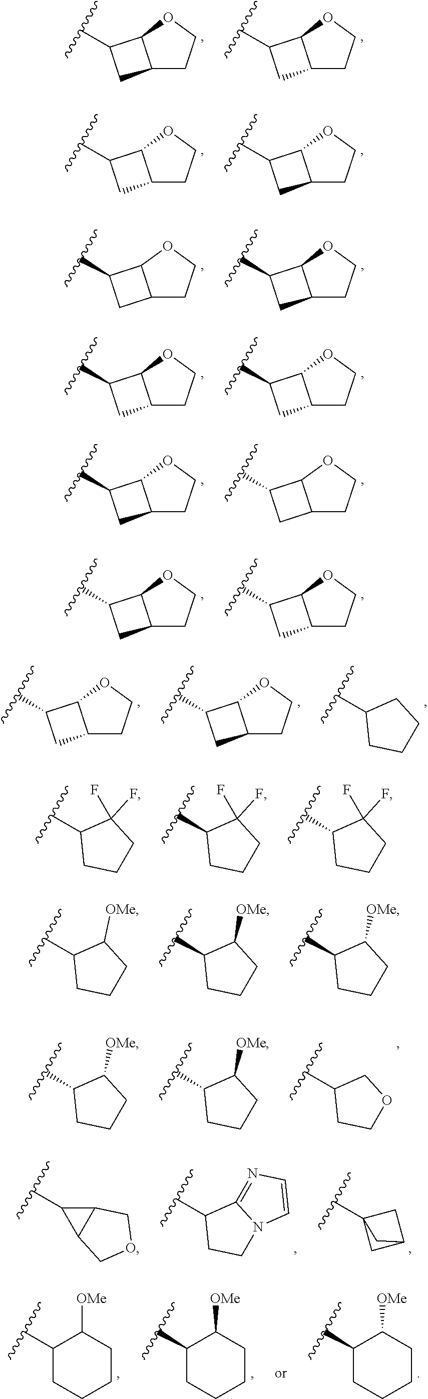

11. A compound of formula I-b 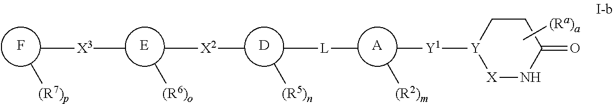
or a pharmaceutically acceptable salt thereof, wherein:
X is a bivalent moiety selected from —CH₂— or —C(O)—;
X² and X³ are independently a covalent bond, —O—, or —NR—;
Y is N or CH;
Y¹ is a covalent bond, —O—, —S—, —NR—, or —C(O)NR—;
Ring A is a ring selected from phenylenyl, pyridinylenyl, 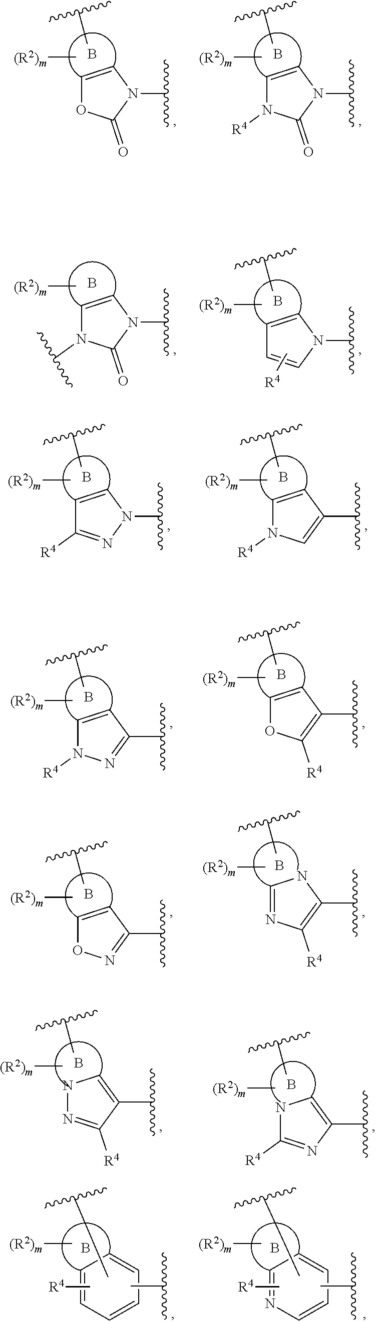 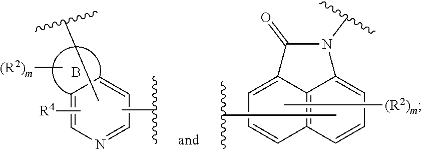
Ring B is a fused ring selected from benzo, a 5-6 membered saturated or partially unsaturated heterocyclyl having 1-3 heteroatoms independently selected from nitrogen, oxygen, and sulfur, or a 5-6 membered heteroaryl containing 1-4 heteroatoms independently selected from nitrogen, oxygen, and sulfur;
Ring D is a bivalent ring selected from phenylenyl, 4-7 membered saturated or partially unsaturated carbocyclylenyl or heterocyclylenyl having 1-2 heteroatoms independently selected from nitrogen, oxygen, and sulfur, and a 5-6 membered heteroarylenyl ring having 1-4 heteroatoms independently selected from nitrogen, oxygen, and sulfur;
Ring E is a bivalent ring selected from phenylenyl, naphthylenyl, 4-7 membered saturated or partially unsaturated carbocyclylenyl or heterocyclylenyl having 1-2 heteroatoms independently selected from nitrogen, oxygen, and sulfur, 5-6 membered heteroarylenyl ring having 1-4 heteroatoms independently selected from nitrogen, oxygen, and sulfur, 9-10 membered saturated or partially unsaturated bicyclic heterocyclylenyl having 1-4 heteroatoms independently selected from nitrogen, oxygen, and sulfur, and 8-10 membered bicyclic heteroarylenyl having 1-4 heteroatoms independently selected from nitrogen, oxygen, and sulfur;
Ring F is phenyl, 4-7 membered saturated or partially unsaturated carbocyclyl or heterocyclyl having 1-2 heteroatoms independently selected from nitrogen, oxygen, and sulfur, and a 5-6 membered heteroaryl ring having 1-4 heteroatoms independently selected from nitrogen, oxygen, and sulfur;
R^{a} is hydrogen, halogen, —CN, —OR, oxo, C₁₋₆alkyl, C₁₋₆ haloalkyl, C₃₋₆ cycloalkyl, or:
two R^{a} on the same carbon atom or adjacent carbon atoms connect to form a 3-6 membered saturated carbocyclic or heterocyclic ring having 1-2 heteroatoms selected from nitrogen, oxygen, and sulfur;
a is 0, 1, or 2;
each R², R⁵, R⁶, and R⁷ are independently hydrogen, halogen, —CN, —NO₂, —OR, oxo, —SR, —NR₂, —SiR₃, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, or R^{A};
each of m, n, o, and p are independently 0, 1, 2, 3, or 4;
R⁴ is hydrogen, halogen, C₁₋₆ alkyl, C₁₋₆ haloalkyl, C₃₋₆ cycloalkyl, —OC₁₋₆ alkyl, or —OC₁₋₆ haloalkyl;
L is a covalent bond, —CR₂—, —CRF—, —CF₂—, —O—, —NR—, —C(O)—, or a bivalent, saturated or partially unsaturated, straight or branched C₁₋₁₀ hydrocarbon chain, wherein 0-6 methylene units of L are independently replaced by -Cy-, —CR₂—, —CRF—, —CF₂—, —CR(OR)—, —O—, —NR—, —C(O)—;
each -Cy- is independently an optionally substituted bivalent ring selected from phenylenyl, 8-10 membered bicyclic arylenyl, 4-7 membered saturated or partially unsaturated carbocyclylenyl, 6-11 membered saturated or partially unsaturated spirocyclic or bridged bicyclic carbocyclylenyl, 8-10 membered bicyclic saturated or partially unsaturated carbocyclylenyl, 4-7 membered saturated or partially unsaturated heterocyclylenyl having 1-2 heteroatoms independently selected from nitrogen, oxygen, and sulfur, 6-11 membered saturated or partially unsaturated spirocyclic or bridged bicyclic heterocyclylenyl having 1-2 heteroatoms independently selected from nitrogen, oxygen, and sulfur, 8-10 membered bicyclic saturated or partially unsaturated heterocyclylenyl having 1-2 heteroatoms independently selected from nitrogen, oxygen, and sulfur, 5-6 membered heteroarylenyl having 1-4 heteroatoms independently selected from nitrogen, oxygen, and sulfur, and 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur; and
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

12. The compound of claim 11, wherein the compound is of any one of the following formulae: 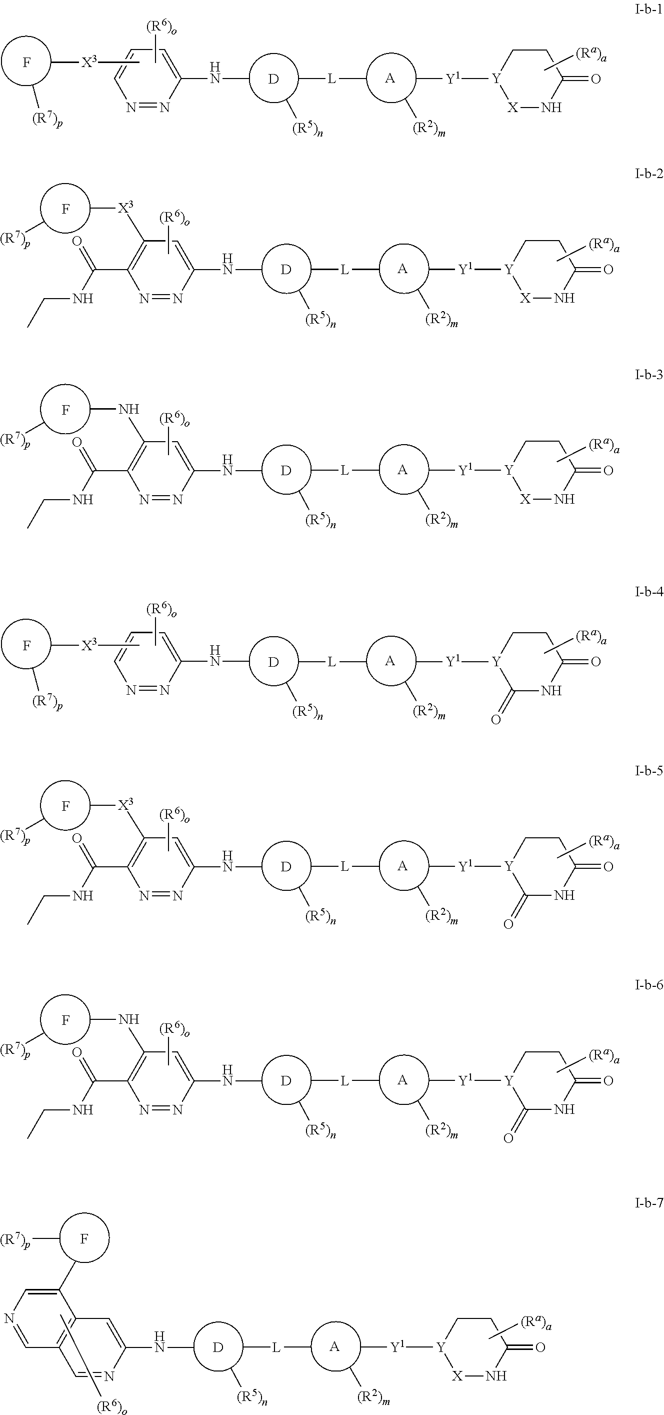 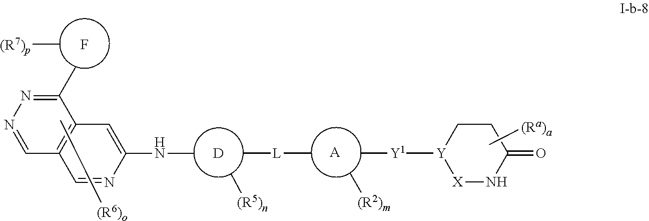
or a pharmaceutically acceptable salt thereof.

13. The compound of claim 11 or claim 12, wherein Ring F is phenyl or a 5-6 membered heteroaryl ring having 1-4 heteroatoms independently selected from nitrogen, oxygen, and sulfur.

14. A compound of formula I-c: 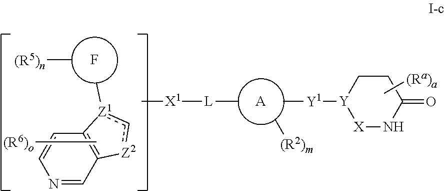
or a pharmaceutically acceptable salt thereof, wherein:
X is a bivalent moiety selected from —CH₂— or —C(O)—;
X¹ is a covalent bond, 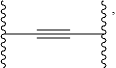 [SMILES: *C#C*]
an optionally substituted ring selected from 4-6 membered saturated carbocyclylenyl or heterocyclylenyl having 1-2 heteroatoms independently selected from nitrogen, oxygen, and sulfur, phenylenyl, 5-6 membered heteroarylenyl having 1-2 heteroatoms independently selected from nitrogen, oxygen, and sulfur, and a 5-8 membered saturated bridged bicyclic heterocyclylenyl having 1-2 heteroatoms independently selected from nitrogen, oxygen, and sulfur;
Y is N or CH;
Y¹ is a covalent bond, —O—, —S—, —NR—, or —C(O)NR—;
Z¹ and Z² are independently N or C, where one of Z¹ and Z² is N and the other of Z¹ and Z² is C;
each [symbol] is independently a single or double bond;
Ring A is a ring selected from phenylenyl, pyridinylenyl, 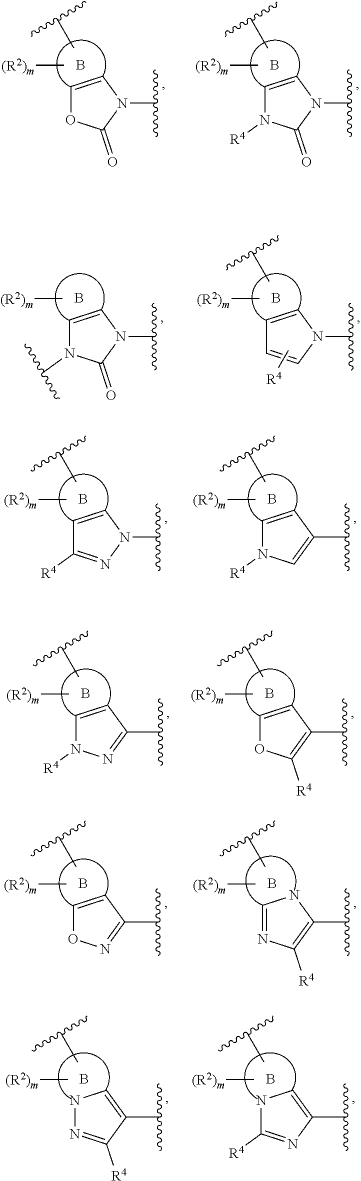 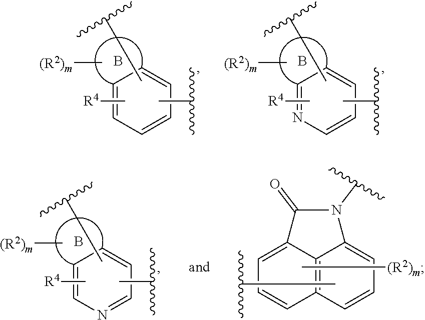
Ring B is a fused ring selected from benzo, a 5-6 membered saturated or partially unsaturated heterocyclyl having 1-3 heteroatoms independently selected from nitrogen, oxygen, and sulfur, or a 5-6 membered heteroaryl containing 1-4 heteroatoms independently selected from nitrogen, oxygen, and sulfur;
Ring F is phenyl, 4-7 membered saturated or partially unsaturated carbocyclyl or heterocyclyl having 1-2 heteroatoms independently selected from nitrogen, oxygen, and sulfur, and a 5-6 membered heteroaryl ring having 1-4 heteroatoms independently selected from nitrogen, oxygen, and sulfur;
R^{a} is hydrogen, halogen, —CN, —OR, oxo, C₁₋₆alkyl, C₁₋₆haloalkyl, C₃₋₆cycloalkyl, or:
two R^{a} on the same carbon atom or adjacent carbon atoms connect to form a 3-6 membered saturated carbocyclic or heterocyclic ring having 1-2 heteroatoms selected from nitrogen, oxygen, and sulfur;
a is 0, 1, or 2;
each R², R⁵, and R⁶ are independently hydrogen, halogen, —CN, —NO₂, —OR, oxo, —SR, —NR₂, —SiR₃, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, or R^{A}, or:
two R⁶ on adjacent atoms connect to form a fused optionally substituted 5-6 membered partially saturated or aromatic heterocyclic ring having 1-2 heteroatoms selected from nitrogen, oxygen, and sulfur;
each of m, n, and o are independently 0, 1, 2, 3, or 4;
R⁴ is hydrogen, halogen, C₁₋₆ alkyl, C₁₋₆ haloalkyl, C₃₋₆ cycloalkyl, —OC₁₋₆ alkyl, or —OC₁₋₆ haloalkyl;
L is a covalent bond, —CR₂—, —CRF—, —CF₂—, —O—, —NR—, —C(O)—, or a bivalent, saturated or partially unsaturated, straight or branched C₁₋₁₀ hydrocarbon chain, wherein 0-6 methylene units of L are independently replaced by -Cy-, —CR₂—, —CRF—, —CF₂—, —CR(OR)—, —O—, —NR—, —C(O)—;
each -Cy- is independently an optionally substituted bivalent ring selected from phenylenyl, 8-10 membered bicyclic arylenyl, 4-7 membered saturated or partially unsaturated carbocyclylenyl, 6-11 membered saturated or partially unsaturated spirocyclic or bridged bicyclic carbocyclylenyl, 8-10 membered bicyclic saturated or partially unsaturated carbocyclylenyl, 4-7 membered saturated or partially unsaturated heterocyclylenyl having 1-2 heteroatoms independently selected from nitrogen, oxygen, and sulfur, 6-11 membered saturated or partially unsaturated spirocyclic or bridged bicyclic heterocyclylenyl having 1-2 heteroatoms independently selected from nitrogen, oxygen, and sulfur, 8-10 membered bicyclic saturated or partially unsaturated heterocyclylenyl having 1-2 heteroatoms independently selected from nitrogen, oxygen, and sulfur, 5-6 membered heteroarylenyl having 1-4 heteroatoms independently selected from nitrogen, oxygen, and sulfur, and 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur; and
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

15. The compound of claim 14, wherein said compound is a compound of any of the following formulae: 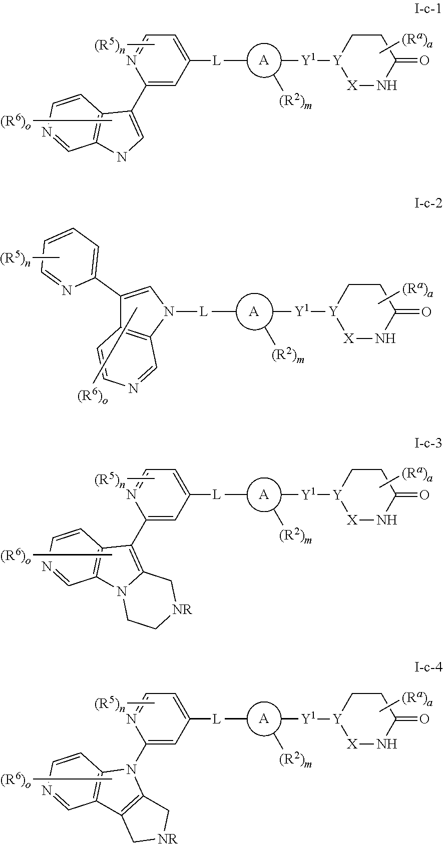
or pharmaceutically acceptable salt thereof.

16. The compound of any one of claims 1-15, wherein

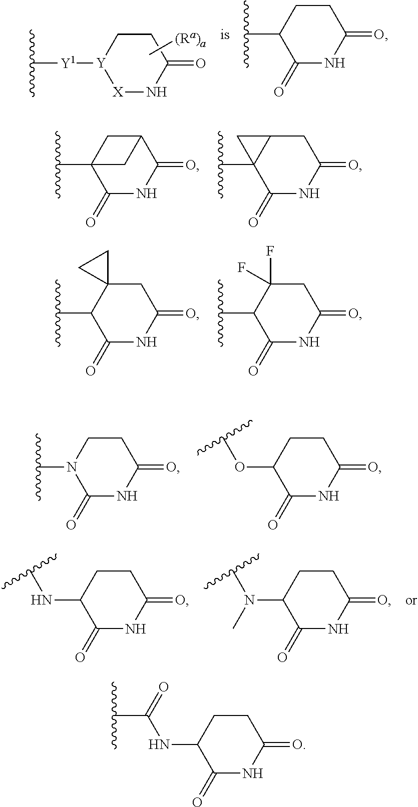

17. The compound of any one of claims 1-16, wherein 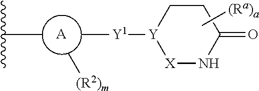
is

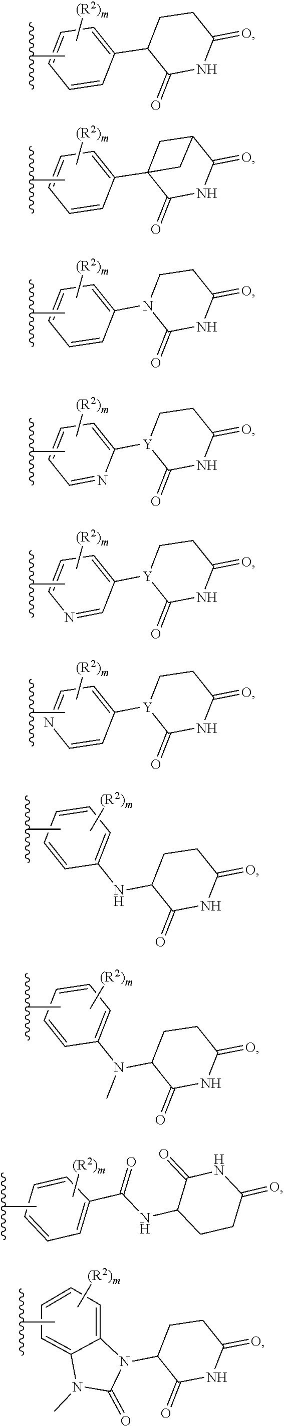

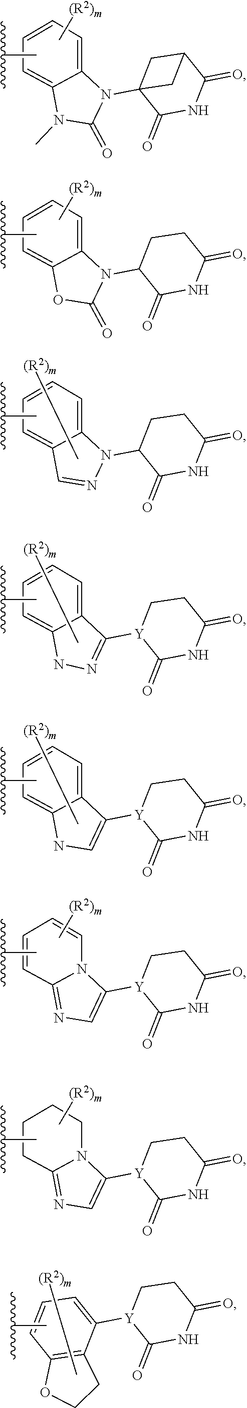

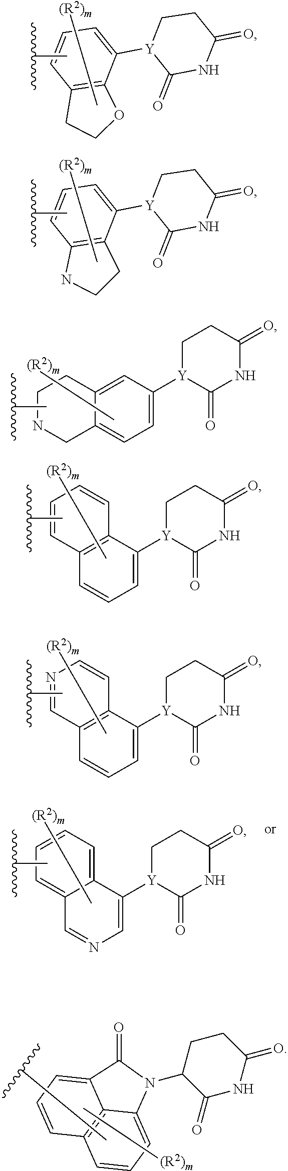

18. The compound of any one of claims 1-17, wherein R² is hydrogen, fluoro, chloro, methyl, ethyl, cyclopropyl, —CHF₂, —CF₃, oxo, —OMe, or -OEt.

19. The compound of any one of claims 1-18, wherein L is a bivalent, saturated or partially unsaturated, straight or branched C₁₋₅ hydrocarbon chain, wherein 0-6 methylene units of L are independently replaced by -Cy-, —CR₂—, —CRF—, —CF₂—, —CR(OR)—, —O—, —NR—, —C(O)—.

20. The compound of any one of claims 1-19, wherein L is a bivalent, saturated or partially unsaturated, straight or branched C₅ hydrocarbon chain, wherein 0-6 methylene units of L are independently replaced by -Cy-, —CR₂—, —CRF—, —CF₂—, —CR(OR)—, —O—, —NR—, —C(O)—.

21. The compound of any one of claims 1-20, wherein L comprises one -Cy- group.

22. The compound of any one of claims 1-21, wherein L comprises two -Cy- groups.

23. The compound of any one of claims 1-22, wherein L comprises 1-3 methylenes, —C(O)—, a 4-7 membered saturated or partially unsaturated heterocyclylenyl having 1-2 heteroatoms independently selected from nitrogen, oxygen, and sulfur, optionally substituted with 1-2 halogens, and a 6-11 membered saturated or partially unsaturated spirocyclic or bridged bicyclic heterocyclylenyl having 1-2 heteroatoms independently selected from nitrogen, oxygen, and sulfur, optionally substituted with 1-2 halogens.

24. The compound of any one of claims 1-23, wherein X¹ is a covalent bond.

25. The compound of any one of claims 1-24, wherein X¹ and X² are covalent bonds.

26. The compound of any one of claims 1-25, wherein X¹, X², and Y¹ are covalent bonds.

27. The compound of any one of claims 1-26, wherein X¹, X², and Y¹ are covalent bonds, and L comprises one -Cy- group.

28. The compound of any one of claims 1-27, wherein X¹, X², and Y¹ are covalent bonds, and L comprises two -Cy- groups.

29. The compound of any one of claims 1-28, wherein X¹, X², and Y¹ are covalent bonds, and L comprises 1-3 methylenes, —C(O)—, a 4-7 membered saturated or partially unsaturated heterocyclylenyl having 1-2 heteroatoms independently selected from nitrogen, oxygen, and sulfur, optionally substituted with 1-2 halogens, and a 6-11 membered saturated or partially unsaturated spirocyclic or bridged bicyclic heterocyclylenyl having 1-2 heteroatoms independently selected from nitrogen, oxygen, and sulfur, optionally substituted with 1-2 halogens.

30. The compound of any one of claims 1-29, wherein L is —O—(C₁₋₅ aliphatic)-, -Cy-(C₁₋₅ aliphatic)-, —(CH₂)₁₋₅-Cy-(C₁₋₅ aliphatic)-, —(CH₂)₁₋₅-Cy-O—(C₁₋₅ aliphatic)-, —(CH₂)₁₋₅-Cy-Cy-(C₁₋₅ aliphatic)-, —(CH₂)₁₋₅-Cy-Cy-O—(C₁₋₅ aliphatic)-, —(CH₂)₁₋₅-Cy-, —(CH₂)₁₋₅-Cy-CH(OR)—, —(CH₂)₁₋₅-Cy-(CH₂)₁₋₅—, —O—(CH₂)₁₋₅-Cy-, —O—(CH₂)₁₋₅-Cy-(CH₂)₁₋₅—, —(CH₂)₁₋₅—O-Cy-, —(CH₂)₁₋₅—O-Cy-(CH₂)₁₋₅—, —(CH₂)₅—CO-Cy-(CH₂)₁₋₅—, —(CH₂)₁₋₅—NR-Cy-, —(CH₂)₁₋₅—NR-Cy-CO—, —(CH₂)₁₋₅-Cy-NR—, —O-Cy-NR—, —(CH₂))₁₋₅—NR-Cy-(CH₂)₁₋₅—, —(CH₂)₁₋₅—CO—, —(CH₂)₁₋₅-Cy-CO—, -Cy-CO—, —O-Cy-CO—, —O-Cy-(CH₂)₁₋₅—, —(CH₂)₁₋₅-Cy-CHF—, —(CH₂)₁₋₅-Cy-CF₂—, —(CH₂)₁₋₅-Cy-(CH₂)₁₋₅—CF₂—, -Cy-Cy-, —(CH₂)₁₋₅-Cy-Cy-, —(CH₂)₁₋₅—Cy-Cy-CO—, -Cy-(CH₂)₁₋₅—Cy-, -Cy-(CH₂)₁₋₅-Cy-CO—, —(CH₂)₁₋₅-Cy-Cy-CH—CO—, -Cy-(CH₂)₁₋₅-Cy-(CH₂)₁₋₅—, —(CH₂)₁₋₅-Cy-Cy-(CH₂)₁₋₅—, —O—(CH₂)₁₋₅-Cy-Cy-, —O—(CH₂)₁₋₅-Cy-Cy-(CH₂)₁₋₅—, —(CH₂)₁₋₅—O-Cy-Cy-, —(CH₂)₁₋₅—O-Cy-Cy-(CH₂)₁₋₅—, —(CH₂)₁₋₅—NR-Cy-Cy-, —(CH₂)₁₋₅-Cy-NR-Cy-, —(CH₂)₁₋₅—NR-Cy-Cy-(CH₂)₁₋₅—, —(CH₂)₁₋₅-Cy-Cy-CO—, —O-Cy-Cy-CO—, —O-Cy-Cy-(CH₂)₁₋₅—, -Cy-Cy-Cy-, -Cy-Cy-Cy-CO—, —(CH₂)₁₋₅-Cy-Cy-Cy-, —(CH₂)₁₋₅-Cy-Cy-Cy-CO—, -Cy-(CH₂)₁₋₅-Cy-Cy-, -Cy-(CH₂)₁₋₅-Cy-Cy-CO—, -Cy-(CH₂)₁₋₅-Cy-(CH₂)₁₋₅-Cy-, or -Cy-(CH₂)₁₋₅-Cy-(CH₂)₁₋₅-Cy-CO.

31. The compound of any one of claims 1-30, wherein -Cy- is

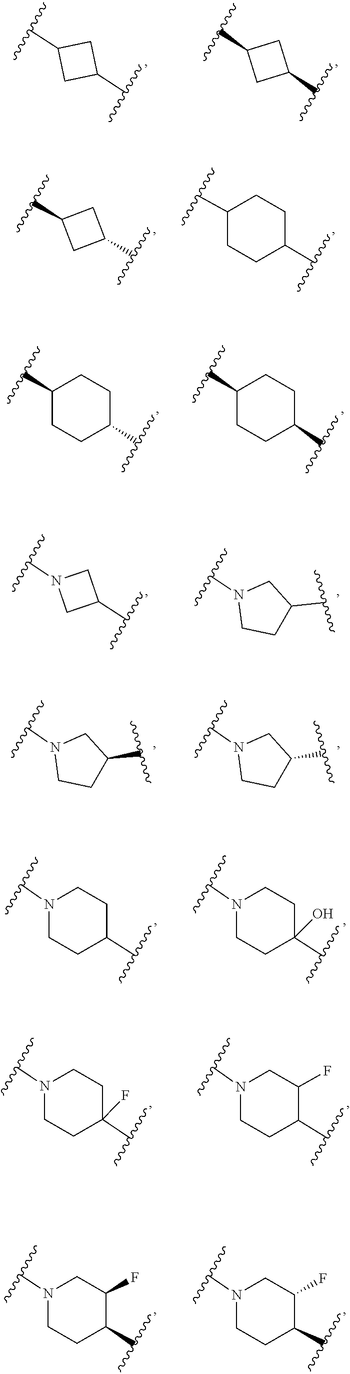

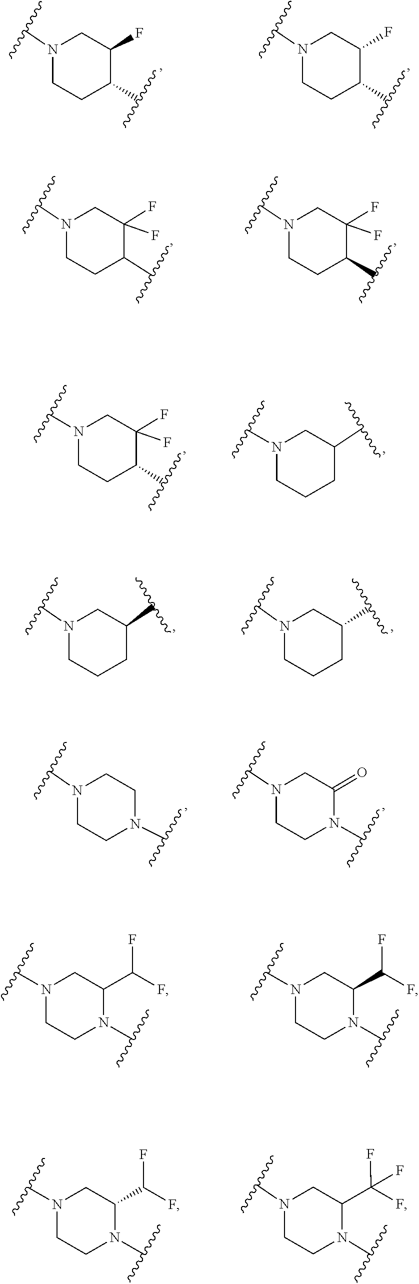

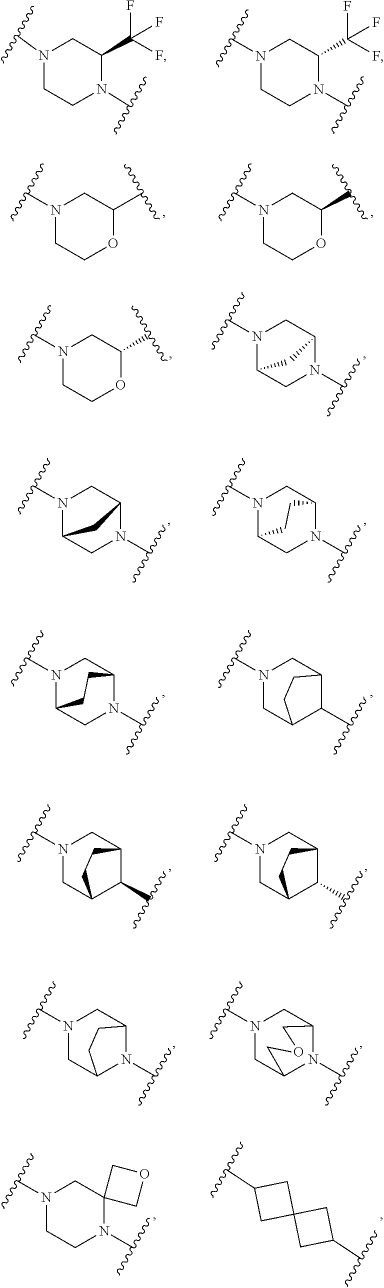

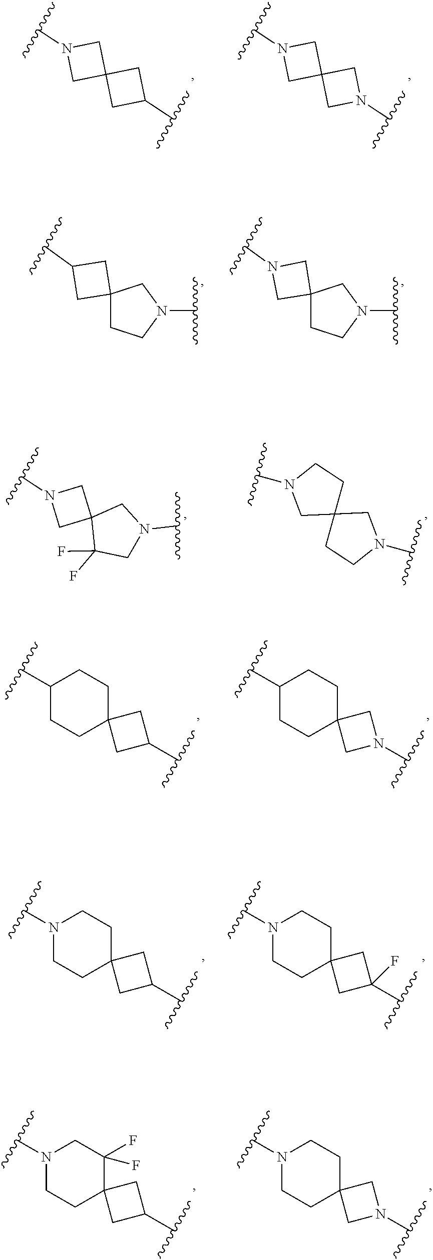

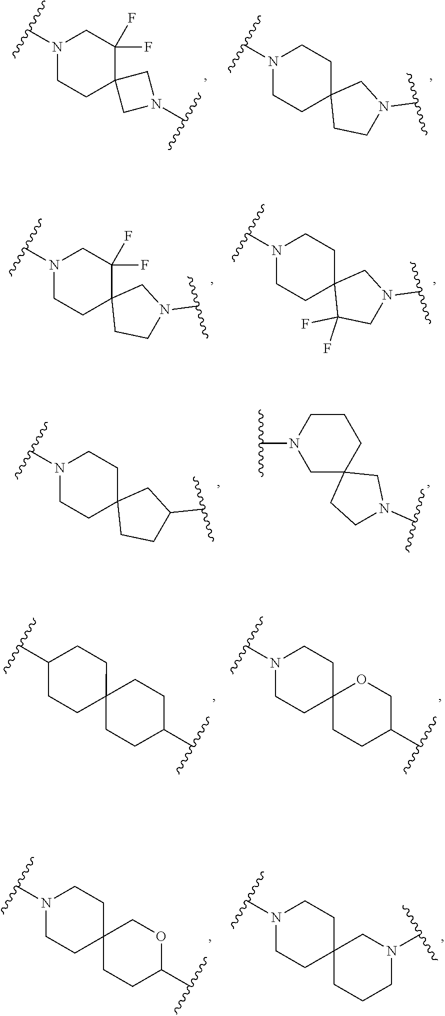

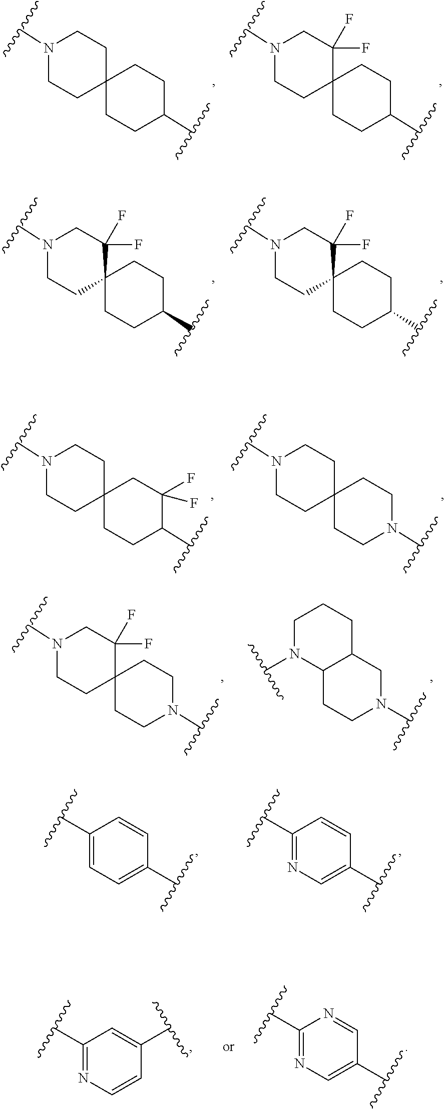

32. The compound of any one of claims 1-31, wherein said compound is selected from any one of the compounds depicted in Table 1, or a pharmaceutically acceptable salt thereof.

33. A pharmaceutical composition comprising a compound of any one of claims 1-32, and a pharmaceutically acceptable carrier, adjuvant, or vehicle.

34. A method of inhibiting or degrading TYK2 in a patient or biological sample comprising administering to said patient, or contacting said biological sample with a compound of any one of claims 1-32, or a pharmaceutical composition thereof.

35. A method of treating a TYK2-mediated disorder, disease, or condition in a subject in need thereof comprising administering to the subject the compound of any of one claims 1-32, or a pharmaceutical composition thereof.

36. The method of claim 35, wherein the TYK2-mediated disorder is an autoimmune disorders, inflammatory disorders, proliferative disorders, cardiovascular disease, endocrine disorders, neurological disorders, dysplasia related disorders, genetic disorders, and disorders associated with transplantation.

37. The method of claim 35, wherein the TYK2-mediated disorder is:
(a) an autoimmune disorder selected from type 1 diabetes, ankylosing spondylitis, cutaneous lupus erythematosus, systemic lupus erythematosus, lupus nephritis, multiple sclerosis, systemic sclerosis, psoriasis, Crohn's disease, ulcerative colitis, proteasome-associated autoinflammatory syndromes (PRAAS), ISG15 deficiency, and inflammatory bowel disease;
(b) an inflammatory disorder selected from rheumatoid arthritis, asthma, chronic obstructive pulmonary disease, psoriasis, Crohn's disease, ulcerative colitis, STING-associated vasculopathy with onset in infancy (SAVI), and inflammatory bowel disease;
(c) a proliferative disorder selected from a hematological cancer, polycythemia vera, myelofibrosis, essential thrombocythemia, and thrombocytosis;
(d) an endocrine disorder selected from polycystic ovary syndrome, Crouzon's syndrome, and type 1 diabetes;
(e) a neurological disorder selected from Alzheimer's disease, Aicardi-Goiuteieres syndrome (AGS), Parkinson's disease, amyotrophic lateral sclerosis, Huntington's disease, cerebral ischemia, and neurodegenerative disease caused by traumatic injury, glutamate neurotoxicity and hypoxia; or
(f) a disorder is associated with transplantation selected from transplant rejection and graft versus host disease.
